(19) United States
(12) Patent Application Publication
(10) Pub. No.: US 2026/0234247 A1
(43) Pub. Date: Aug. 13, 2026

(54) ANTI-LILRB1 AND/OR ANTI-LILRB2 ANTIBODIES AND USES THEREOF

(71) Applicant: LAEKNA PHARMACEUTICAL NINGBO CO., LTD., Ningbo (CN)

(72) Inventors: Rong WU, Shanghai (CN); Chaojun CAI, Shanghai (CN); Minhua ZHANG, Shanghai (CN); Xiang-Ju Justin GU, Shanghai (CN)

(73) Assignee: LAEKNA PHARMACEUTICAL NINGBO CO., LTD., Ningbo, ZJ (CN)

(21) Appl. No.: 19/152,905

(22) PCT Filed: Feb. 1, 2024
(86) PCT No.: PCT/CN2024/075347

§ 371 Date: Jul. 31, 2025

(30) Foreign Application Priority Data
Feb. 2, 2023 (WO) PCT/CN2023/074276

(51) Int. Cl. C07K 16/28; A61P 37/02

(52) U.S. Cl. CPC C07K 16/2803; A61P 37/02; C07K 16/2809; C07K 16/2818; C07K 2317/31; C07K 2317/622

(57) ABSTRACT

Provided herein, in certain aspects, are antibodies that bind to LILRB1, and uses thereof. Also provided herein, in certain aspects, are antibodies that bind to LILRB2, and uses thereof. Also provided herein, in certain aspects, are multispecific antibodies that bind to LILRB1 and LILRB2, and uses thereof.

### CROSS-REFERENCE TO RELATED APPLICATIONS

This application claims priority to International Patent Application No. PCT/CN2023/074276, filed Feb. 2, 2023, the disclosure of which is incorporated by reference herein in its entirety.

### SEQUENCE LISTING

This application contains a computer readable Sequence Listing which has been submitted in XML file format with this application, the entire content of which is incorporated by reference herein in its entirety. The Sequence Listing XML file submitted with this application is entitled “14668-026-228_SEQ_LISTING.xml”, was created on Jan. 31, 2024, and is 1,480,704 bytes in size.

### 1. FIELD

The present disclosure relates generally to binding agents, such as antibodies (including fragments thereof) that bind to LILRB1 and/or LILRB2, including anti-LILRB1 antibodies (including humanized anti-LILRB1 antibodies), anti-LILRB2 antibodies (including humanized anti-LILRB2 antibodies), and multispecific antibodies that bind to both LILRB1 and LILRB2 (including humanized multispecific antibodies), and methods of use thereof.

### 2. BACKGROUND

Leukocyte immunoglobulin-like receptor subfamily B member 1 (LILRB1), also called Ig-like transcript 2 (ILT2), is an immunoreceptor tyrosine-based inhibitory motif-containing receptor for class I major histocompatibility complex (MHC) antigens and recognizes a broad spectrum of human leukocyte antigen (HLA)-A, HLA-B, HLA-C and HLA-G alleles. LILRB1 use its two membrane distal domains (D1 and D2) to recognize the a3 domain and b2m subunit of MHC molecules. LILRB1 is also a receptor for H301/UL18, a human cytomegalovirus class I MHC homolog (Yu et al. (2018) J Clin Invest 1523-1537). LILRB1 is expressed on all B cells, monocytes, macrophages, dendritic cells, myeloid-derived suppressor cells (MDSC) and subsets of NK cells and T cells and transduce a negative signal that downregulates the immune response (Chen et al. (2020) J Immunother Cancer e000515).

Leukocyte immunoglobulin-like receptor subfamily B member 2 (LILRB2), also called Ig-like transcript 4 (ILT4), is an immunoreceptor tyrosine-based inhibitory motif-containing receptor for class I MHC antigens and recognizes a broad spectrum of HLA-A, HLA-B, HLA-C and HLA-G alleles. LILRB2 use its two membrane distal domains (D1 and D2) to recognize the a3 domain of MHC molecules. Angiopoietin-like proteins (ANGPTLs), CD1d, Ab oligomers and myelin inhibitors are also ligands for LILRB2 (Zhang et al. (2017) J Leukoc Biol 351-360). Ligand binding results in inhibitory signals and down-regulation of the immune response. LILRB2 is expressed on monocytes, macrophages, dendritic cells, granulocytes and myeloid-derived suppressor cells (MDSC) (Siu et al. (2021) Clin Cancer Res 57-60).

LILRB1 and LILRB2 suppresses immune activation by binding MHC-I in cis and trans. They recruit SHP-1 and SHP-2 phosphatases which mediate inhibition of various intracellular signal pathways (Chen et al. (2018) J Clin Invest 5647-5662). Both LILRB1 and LILRB2 are associated with advanced disease stage and unfavorable prognosis in multiple tumor types (Chen et al. (2022) Int Immunopharmacol 108798 and Zhang et al. (2021) Front Oncol 668707). HLAs play a vital role in cancer immunology. A strategy used by tumor cells to escape innate and adaptive immune response is associated with aberrant expression of human leukocyte antigen (HLA)-G (Carosella et al. (2015) Adv Immunol 33-144). HLA-A, HLA-B, HLA-C and HLA-G binding to LILRB1 and LILRB2 can directly inhibit immune cell function through receptor binding and/or through phagocytosis and impairment of chemotaxis (Morandi et al. (2014) Cytokine Growth Factor Rev 327-335). HLA-G is high expressed in multiple tumor types and associated with poor prognosis (Carosella et al. (2015) Adv Immunol 33-144). The interaction between HLAs and macrophages due to LILRB1 inhibits phagocytic function (Barkal et al. (2018) Nat Immunol 76-84). HLAs binding to LILRB1 can inhibit cytotoxic activity of NK and T cells (Dumont et al. (2019) Cancer Immunol Res 1619-1632 and Chen et al. (2020) J Immunother Cancer e000515). The interaction between HLAs and monocytes due to LILRB2 inhibits maturation of human monocyte-derived antigen-presenting ceils (APCs) (Liang et al. (2008) Proc Natl Acad Sci USA 8357-8362). HLAs binding to LILRB1 and LILRB2 can suppress immune system by inducing dendritic cells and MDSC (Zhang et al. (2017) J Leukoc Biol 351-360).

Many patients do not respond to T cell therapy because of tumor immune escape or tumor microenvironment. Some of them may get relief from NK and myeloid cells therapy. Antibody-mediated blockade of LILRB1 and LILRB2 function would fill this unmet medical need by reverse the immunosuppressed tumor microenvironment.

### 3. SUMMARY

The present disclosure provides an antibody or antigen binding fragment thereof that binds LILRB1, e.g., an antibody or antigen binding fragment provided in Tables 4-6, and 13-15.

The present disclosure provides an antibody or antigen binding fragment thereof that binds LILRB2, e.g., an antibody or antigen binding fragment provided in Tables 4-6, and 13-15.

The present disclosure further provides a multispecific antibody or antigen binding fragment thereof comprising a first binding arm that binds to LILRB1 and a second binding arm that binds to LILRB2. The first binding domain contains a VH and a VL comprising CDRs identical to any antibody or fragment thereof provided herein, for example, in Tables 5-6, and 14-15. The second binding domain contains a VH and a VL comprising CDRs identical to any antibody or fragment thereof provided herein, for example, in Tables 5-6, and 14-15.

In some embodiments, the multispecific antibody or fragment thereof provided herein has an antibody format of immunoglobulin-single chain antibody fragment (IgG-ScFv). In some embodiments, the multispecific antibody or fragment thereof provided herein has an antibody format of dual-variable-domain-immunoglobulin (DVD-Ig).

In some embodiments, the antibody provided herein is an IgG. In some embodiments, the antibody is a humanized antibody.

In another aspect, provided herein is a nucleic acid molecule encoding the antibody or antigen binding fragment provided herein.

In another aspect, provided herein is a vector comprising the nucleic acid molecule encoding the antibody or antigen binding fragment provided herein.

In yet another aspect, provided herein is a host cell transformed with the vector encoding the antibody or antigen binding fragment provided herein.

In yet another aspect, provided herein is a composition comprising a therapeutically effective amount of the antibody or antigen binding fragment, the nucleic acid molecule, or the vector encoding the antibody or antigen binding fragment provided herein, and a pharmaceutically acceptable excipient.

In yet another aspect, provided herein is a method of preventing suppression of an immune cell or activating a response mediated by an immune cell, comprising contacting the immune cell with the antibody or fragment thereof provided herein or the pharmaceutical composition provided herein. In some embodiments, the immune cell is an NK cell, a monocyte, a macrophage, a T cell or a dendric cell. In some embodiments, the T cell is a CD8+ T cell. In some embodiments, the immune cell expresses LILRB1 and/or LILRB2. In some embodiments, the response mediated by the immune cell is an anti-tumor response. In some embodiments, the tumor cell expresses HLA-A2, ANGPTLs and/or HLA-G.

In yet another aspect, provided herein is a method for treating a disease or disorder in a subject comprising administering to the subject the antibody or antigen binding fragment provided herein, or the pharmaceutical composition provided herein. In some embodiments, the disease or disorder is a cancer. In some embodiments, the cancer expresses HLA-A2, ANGPTLs and/or HLA-G. In some embodiments, the disease or disorder is a solid tumor or a blood tumor. In some embodiments, the subject is a human subject.

In some embodiments, the antibody or antigen binding fragment is used as part of a combination therapy. In some embodiments, the antibody or antigen binding fragment is used in combination with a PD-1 inhibitor. In some embodiments, the PD-1 inhibitor is an anti-PD-1 antibody. In some embodiments, the anti-PD-1 antibody is Nivolumab. In some embodiments, the antibody or antigen binding fragment is used in combination with a PD-L1 inhibitor. In some embodiments, the PD-L1 inhibitor is an anti-PD-L1 antibody. In some embodiments, the anti-PD-L1 antibody is LAE005. In some embodiments, the antibody or antigen binding fragment is used in combination with an anti-CD47 antibody. In some embodiments, the anti-CD47 antibody is Hu5F9-G4. In some embodiments, the antibody or antigen binding fragment is used in combination with an anti-CD3 antibody. In some embodiments, the anti-CD3 antibody is OKT3.

### 4. BRIEF DESCRIPTION OF THE FIGURES

FIGS. 1A and 1B show the ability of exemplary anti-LILRB1 antibodies and anti-LILRB2 antibodies to block the interactions between LILRB1 or LILRB2 expressed at the surface of cell lines and APC-HLA-A2/Human MLANA (AAGIGILTV (SEQ ID NO: 1285)) MHC Tetramer as assessed by flow cytometry. Anti-LILRB1 antibodies LK-RB1-3, LK-RB1-9, LK-RB1-12, LK-RB1-16 and LK-RB1-39 all blocked the interaction of LILRB1 with HLA-A2 (FIG. 1A). Anti-LILRB2 antibodies LK-RB2-24, LK-RB2-1, LK-RB2-2, LK-RB2-3, LK-RB2-14 and LK-RB2-15 all blocked the interaction of LILRB2 with HLA-A2 (FIG. 1B).

FIGS. 2A and 2B show LK-RB1-9 bind to human LILRA1 with low affinity. FIG. 2A shows the ability of hz73D1.v1, LK-RB1-9 and 15G8 for binding to CHOK1-human LILRA2 cell line by flow cytometry. FIG. 2B shows the ability of hz73D1.v1, LK-RB1-9 and 15G8 for binding to human LILRA1 protein by ELISA.

FIGS. 3A-3E show dose dependent binding of the exemplary anti-LILRB1 antibodies and anti-LILRB2 antibodies to cynomolgus LILRB1 and cynomolgus LILRB2. FIG. 3A shows dose dependent binding of anti-LILRB1 antibodies LK-RB1-43, LK-RB1-11, LK-RB1-50, LK-RB1-58, and hz73D1.v1 to cynomolgus LILRB1 protein by ELISA. FIG. 3B shows dose dependent binding of anti-LILRB2 antibodies LK-RB2-30, LK-RB2-31 and hz73D1.v1 to cynomolgus LILRB1 protein by ELISA. FIGS. 3C-3E show dose dependent binding of exemplary anti-LILRB2 antibodies to cynomolgus LILRB2/cynomolgus LILRB2-Flag overexpressing CHOK1 cell line by flow cytometry.

FIGS. 4A-4K show humanized anti-LILRB1 and anti-LILRB2 antibodies bind to human LILRB1 and/or LILRB2 and block the interactions between human LILRB1/2 and HLA-G with high affinity. FIGS. 4A and 4B show the ability of LK-RB1-9, LK-RB1-9.h1, LK-RB1-9.h2, LK-RB1-9.h3, LK-RB1-16, LK-RB1-16.h1, LK-RB1-16.h2 and LK-RB1-16.h3 for binding to CHOK1-human LILRB1 cell line by flow cytometry. FIGS. 4C and 4D show the ability of LK-RB1-9, LK-RB1-9.h1, LK-RB1-9.h2, LK-RB1-9.h3, LK-RB1-16, LK-RB1-16.h1, LK-RB1-16.h2 and LK-RB1-16.h3 to block the interactions between human LILRB1 and PE-HLA-G/Human H2AFX (RIIPRHLQL (SEQ ID NO: 1284)) MHC tetramer as assessed by flow cytometry. FIG. 4E shows dose dependent binding of LK-RB1-9, LK-RB1-9.h1, LK-RB1-9.h2 and LK-RB1-9.h3 to CHOK1-human LILRA2 cell line by flow cytometry. FIGS. 4F and 4G show the ability of LK-RB2-2, LK-RB2-2.h1, LK-RB2-2.h2, LK-RB2-2.h3, LK-RB2-24, LK-RB2-24.h1, LK-RB2-24.h2 and LK-RB2-24.h3 for binding to CHOK1-human LILRB2 cell line by flow cytometry. FIGS. 4H-4I show the ability of LK-RB2-2, LK-RB2-2.h1, LK-RB2-2.h2, LK-RB2-2.h3, LK-RB2-24, LK-RB2-24.h1, LK-RB2-24.h2 and LK-RB2-24.h3 to block the interactions between human LILRB2 and PE-HLA-G/Human H2AFX (RIIPRHLQL (SEQ ID NO: 1284)) MHC tetramer as assessed by flow cytometry. FIG. 4J shows dose dependent binding of LK-RB2-24, LK-RB2-24.h1, LK-RB2-24.h2 and LK-RB2-24.h3 to CHOK1-human LILRB1 cell line by flow cytometry. FIG. 4K shows dose dependent ability of LK-RB2-24, LK-RB2-24.h1, LK-RB2-24.h2 and LK-RB2-24.h3 to block the interactions between human LILRB1 and PE-HLA-G/Human H2AFX (RIIPRHLQL (SEQ ID NO: 1284)) MHC tetramer as assessed by flow cytometry.

FIGS. 5A-5D show bispecific antibodies LK-RBi-05 and LK-RBi-08 bind to human LILRB1/2 and block the interactions between human LILRB1/2 and HLA-G with high affinity. FIG. 5A shows dose dependent binding of LK-RBi-05, LK-RBi-08, LK-RB1-9.h1, LK-RB1-16.h3, 15G8 and hz73D1.v1 to human LILRB1 by flow cytometry. FIG. 5B shows the ability of LK-RBi-05, LK-RBi-08, LK-RB1-9.h1, LK-RB1-16.h3, 15G8 and hz73D1.v1 to block the interactions between human LILRB1 and PE-HLA-G/Human H2AFX (RIIPRHLQL (SEQ ID NO: 1284)) MHC tetramer as assessed by flow cytometry. FIG. 5C shows dose dependent binding of LK-RBi-05, LK-RBi-08, LK-RB2-2.h1, J19.h1 and hz73D1.v1 to human LILRB2 by flow cytometry. FIG. 5D show the ability of LK-RBi-05, LK-RBi-08, LK-RB2-2.h1, J19.h1 and hz73D1.v1 to block the interactions between human LILRB2 and PE-HLA-G/Human H2AFX (RIIPRHLQL (SEQ ID NO: 1284)) MHC tetramer as assessed by flow cytometry.

FIGS. 6A-6R show the exemplary anti-LILRB1 antibodies activated LILRB1-positive NK cells with high potency in the co-culture of primary human NK cells and K562 tumor cells expressing HLA-G. FIGS. 6A-6D show % CD107a positive cells in LILRB1-negative NK cells did not increase after the treatment of 0.5 μg/ml and 0.05 μg/ml of anti-LILRB1 antibodies. FIGS. 6E-6H show % CD107a positive cells in LILRB1-positive NK cells increased significantly after the treatment of 0.5 μg/ml and 0.05 μg/ml of anti-LILRB1 antibodies. FIGS. 6I-6K show the lack of dose dependent increase of % CD107a positive cells in LILRB1-negative NK cells after the treatment of anti-LILRB1 antibodies. FIGS. 6L-6N show the dose dependent increase of % CD107a positive cells in LILRB1-positive NK cells after the treatment of anti-LILRB1 antibodies. FIGS. 6O and 6P show the dose dependent effect of % CD107a positive cells in LILRB1-negative NK cells after the treatment of LK-RB1-9, LK-RB1-9.h1, LK-RB1-9.h2, LK-RB1-9.h3, LK-RB1-16, LK-RB1-16.h1, LK-RB1-16.h2 and LK-RB1-16.h3. FIGS. 6Q and 6R show the dose dependent increase of % CD107a positive cells in LILRB1-positive NK cells after the treatment of LK-RB1-9, LK-RB1-9.h1, LK-RB1-9.h2, LK-RB1-9.h3, LK-RB1-16, LK-RB1-16.h1, LK-RB1-16.h2 and LK-RB1-16.h3.

FIGS. 7A-7I show the exemplary anti-LILRB1 antibodies dose dependently increased Hu5F9-G4 (anti-CD47)-induced macrophage phagocytosis in the co-culture of human monocyte-derived macrophages and Raji (HLA-A2-positive) tumor cells. FIGS. 7A-7C show macrophage phagocytosis of Raji cells by Hu5F9-G4 was further increased by 2.5 μg/ml anti-LILRB1 antibodies treatment. FIGS. 7D-7G show the dose dependent increasing of Hu5F9-G4-induced macrophage phagocytosis of Raji cells by anti-LILRB1 antibodies treatment. FIGS. 7H and 7I show the dose dependent increasing of Hu5F9-G4-induced macrophage phagocytosis by LK-RB1-9, LK-RB1-9.h1, LK-RB1-16, LK-RB1-16.h3 and LK-RB1-55 treatment.

FIGS. 8A and 8B show LK-RB1-9 and LK-RB1-9.h1 further increased Nivolumab-induced IFNγ release by human primary CD8+ T cells in the co-culture of primary CD8+ T cells and CHOK1-HLA-G & PDL-1 & OKT3 cells.

FIGS. 9A-9H show the exemplary antibodies enhance LPS-induced TNFα release in human primary monocyte-differentiated macrophages. FIG. 9A shows LK-RB1-9 increased LPS-induce TNFα release in human primary macrophages. FIG. 9B shows LK-RB1-9 did not change IL-10 release in LPS-stimulated human primary macrophages. FIG. 9C shows LK-RB1-9.h1 and LK-RB1-16.h3 increased LPS-induced TNFα release in human primary macrophages. FIGS. 9D-9F show LPS-induced TNFα release was increased significantly after the treatment of 6 μg/ml of anti-LILRB2 antibodies. FIG. 9G shows LK-RB2-2.h1 increased LPS-induced TNFα release in human primary macrophages. FIG. 9H shows LK-RB2-2.h1 reduced LPS-induced IL-10 release in human primary macrophages.

FIGS. 10A-10I show the exemplary anti-LILRB2 antibodies enhance LPS-induced TNFα release in human PBMC. FIGS. 10A and 10B show LPS-induced TNFα release was increased significantly after the treatment of 60 μg/ml of anti-LILRB2 antibodies. FIGS. 10C-10H show the dose dependent increase of LPS-induced TNFα release in human PBMC after the treatment of anti-LILRB2 antibodies. FIG. 10I shows the dose dependent increase of LPS-induced TNFα release in human PBMC after the treatment of LK-RB2-2.h1 and LK-RB2-24.h3.

FIGS. 11A-11F show the exemplary anti-LILRB2 antibodies enhance anti-CD3 antibody-induced TNFα and IFN-γ release in human primary PBMC. FIGS. 11A and 11B show the dose dependent increase of anti-CD3 antibody-induced TNFα release in human PBMC after the treatment of LK-RB2-2, LK-RB2-24 and LK-RB2-15. FIGS. 11C and 11D show the dose dependent increase of anti-CD3 antibody-induced IFN-γ release in human PBMC after the treatment of LK-RB2-2, LK-RB2-24 and LK-RB2-15. FIG. 11E shows the dose dependent increase of anti-CD3 antibody-induced TNFα release in human PBMC after the treatment of LK-RB2-2.h1 and LK-RB2-24.h3. FIG. 11F shows the dose dependent increase of anti-CD3 antibody-induced IFN-γ release in human PBMC after the treatment of LK-RB2-2.h1 and LK-RB2-24.h3.

FIGS. 12A-12C show LK-RB1-9.h1 and LK-RB1-16.h3 bind to human LILRB1 wild type, Variant 2 and Variant 3 with similar affinity. FIG. 12A shows the ability of LK-RB1-9.h1 and LK-RB1-16.h3 for binding to human LILRB1 wild type by ELISA. FIG. 12B shows the ability of LK-RB1-9.h1 and LK-RB1-16.h3 for binding to human LILRB1 Variant 2 by ELISA. FIG. 12C shows the ability of LK-RB1-9.h1 and LK-RB1-16.h3 for binding to human LILRB1 Variant 3 by ELISA.

FIGS. 13A-13H show the dose dependent increase of % CD107a positive cells in NK cells after the treatment of anti-LILRB1 antibodies and anti-LILRB1/LILRB2 bispecific antibodies. FIGS. 13A-13D show the effect of LK-RBi-01, LK-RBi-02, LK-RBi-05, LK-RBi-06, LK-RBi-07, LK-RBi-08, LK-RBi-09, LK-RBi-10, LK-RBi-11, LK-RB1-9.h1, LK-RB1-16.h3, 15G8 and hz73D1.v1 on % CD107a positive cells in LILRB1 negative NK cells. FIGS. 13E-13H show the effect of LK-RBi-01, LK-RBi-02, LK-RBi-05, LK-RBi-06, LK-RBi-07, LK-RBi-08, LK-RBi-09, LK-RBi-10, LK-RBi-11, LK-RB1-9.h1, LK-RB1-16.h3, 15G8 and hz73D1.v1 on % CD107a positive cells in LILRB1 positive NK cells.

FIGS. 14A-14F show the dose dependent increase of phagocytosis in the presence of Hu5F9-G4 after the treatment of bispecific antibodies LK-RBi-01, LK-RBi-02, LK-RBi-05, LK-RBi-06, LK-RBi-07, LK-RBi-08, LK-RBi-09, LK-RBi-10, LK-RBi-11 LK-RB1-9.h1, LK-RB1-16.h3, 15G8 and hz73D1.v1.

FIG. 15 shows LK-RBi-05, LK-RBi-08, LK-RB1-9.h1 and LK-RB1-16.h3 increased LAE005-induced IFNγ release by human primary CD8+ T cells in the co-culture of primary CD8+ T cells and CHOK1-HLA-G & PDL-1 & OKT3 cells.

FIGS. 16A and 16B show the anti-LILRB1 antibodies, anti-LILRB2 antibodies and bispecific anti-LILRB1/LILRB2 antibodies activated LPS-induced human primary monocyte-differentiated macrophages. FIG. 16A shows LK-RB1-9.h1, LK-RB1-16.h3, LK-RB2-2.h1, LK-RBi-05, LK-RBi-08, LK-RB1-9.h1/LK-RB2-2.h1 combination, and LK-RB1-16.h3/LK-RB2-2.h1 combination increased LPS-induced TNFα release in human primary macrophages. FIG. 16B shows LK-RB2-2.h1, LK-RBi-05, LK-RBi-08, LK-RB1-9.h1/LK-RB2-2.h1 combination, and LK-RB1-16.h3/LK-RB2-2.h1 combination reduced IL-10 release in LPS-stimulated human primary macrophages.

FIGS. 17A-17C show the dose dependent increase of LPS-induced TNFα release in human PBMC after the treatment of bispecific anti-LILRB1/LILRB2 antibodies LK-RBi-01, LK-RBi-02, LK-RBi-05, LK-RBi-06, LK-RBi-07, LK-RBi-08 and LK-RBi-10.

FIGS. 18A and 18B show the dose dependent increase of anti-CD3 antibody-induced TNFα release and IFN-γ release in human PBMC after the treatment of bispecific anti-LILRB1/LILRB2 antibodies LK-RBi-05 and LK-RBi-08. FIG. 18A shows LK-RBi-05, LK-RBi-08 and hz73D1.v1 dose dependently increased OKT3-induced TNFα release in human PBMC. FIG. 18B shows LK-RBi-05, LK-RBi-08 and hz73D1.v1 dose dependently increased OKT3-induced IFN-γ release in human PBMC.

FIG. 19 show the exemplary anti-LILRB1 antibodies, anti-LILRB2 antibodies, anti-LILRB1/LILRB2 bispecific antibodies, and antibody combinations increased TNFα release induced by Fc receptors crosslinking in human primary dendritic cells. Bispecific anti-LILIRB1/LILRB2 antibodies LK-RBi-05 and LK-RBi-08 had stronger effect on increasing cytokine production comparing to anti-LILRB1 antibody LK-RB1-9.h1, LK-RB1-16.h3 and anti-LILRB2 antibody LK-RB2-2.h1.

### 5. DETAILED DESCRIPTION

The present disclosure is based in part on the novel antibodies that bind to LILRB1 and/or LILRB2 and superior properties thereof.

### 5.1. Definitions

Techniques and procedures described or referenced herein include those that are generally well understood and/or commonly employed using conventional methodology by those skilled in the art, such as, for example, the widely utilized methodologies described in Sambrook et al., Molecular Cloning: A Laboratory Manual (3d ed. 2001); Current Protocols in Molecular Biology (Ausubel et al. eds., 2003); Therapeutic Monoclonal Antibodies: From Bench to Clinic (An ed. 2009); Monoclonal Antibodies: Methods and Protocols (Albitar ed. 2010); and Antibody Engineering Vols 1 and 2 (Kontermann and Dübel eds., 2d ed. 2010). Unless otherwise defined herein, technical and scientific terms used in the present description have the meanings that are commonly understood by those of ordinary skill in the art. For purposes of interpreting this specification, the following description of terms will apply and whenever appropriate, terms used in the singular will also include the plural and vice versa. In the event that any description of a term set forth conflicts with any document incorporated herein by reference, the description of the term set forth below shall control.

The term “LILRB1” also known as CD85J, LIR1, ILT2, is a single pass type I transmembrane protein with a predicted molecular weight of approximately 71 kDa. LILRB1 (human, rhesus, and cyno) is characterized by an extracellular domain comprising four Ig-like C2 type domains, a transmembrane domain, and a long cytoplasmic domain containing 4 ITIM domains (see, e.g., Borges et al., 1997, J. Immunol., 159:5192-5196). The four Ig-like C2-type domains may be referred to herein as Domain 1 (D1), Domain 2 (D2), Domain 3 (D3), and Domain 4 (D4). D1 is situated at the N-terminal portion of the protein, then D2, D3, with D4 situated closest to the transmembrane region. As characterized within UniProtKB, human LILRB1 is a protein of 650 amino acids (aa)—the signal sequence is aa 1-23, the extracellular domain is aa 24-461, the transmembrane region is aa 462-482, and the cytoplasmic domain is aa 483-650. Within the extracellular domain, D1 is aa 27-115, D2 is aa 116-221, D3 is aa 222-312, D4 is aa 313-409, and the “stem region” is aa 410-461. Within the cytoplasmic domain, ITIMs are aa 531-536, 560-565, 612-617, and 642-647. LILRB1 is expressed (to varying degrees) on natural killer (NK) cells, monocytes, macrophages, eosinophils, basophils, dendritic cells (DCs), subset of T-cells, and B-cells. Various ligands are known to interact with LILRB1, including HLA class I molecules (e.g., HLA-A, HLA-B, HLA-C, HLA-E, HLA-F, and HLA-G).

The term “LILRB2” also known as CD85D, LIR2, ILT4, is a single pass type I transmembrane protein with a predicted molecular weight of approximately 65 kDa. ILT4 is characterized by an extracellular domain comprising four Ig-like C2 type domains, a transmembrane domain, and a long cytoplasmic domain containing 3 ITIM domains (see, e.g., Borges et al., 1997, J. Immunol., 159:5192-5196). As described for ILT2, the four Ig-like C2-type domains may be referred to herein as D1, D2, D3, and D4. D1 is situated at the N-terminal portion of the protein, then D2, D3, with D4 situated closest to the transmembrane region. As characterized within UniProtKB, human ILT4 is a protein of 598 amino acids (aa)—the signal sequence is aa 1-21, the extracellular domain is aa 22-461, the transmembrane region is aa 462-482, and the cytoplasmic domain is aa 483-598. Within the extracellular domain, D1 is aa 27-110, D2 is aa 111-229, D3 is aa 230-318, D4 is aa 330-419, and the “stem region” is aa 420-461. Within the cytoplasmic domain, ITIMs are aa 531-536, 560-565, and 590-595. ILT4 is expressed on myeloid cells such as monocytes, macrophages, dendritic cells, but not on lymphoid cells. ILT4 has been observed to bind a variety of ligands, notably HLA class I molecules, ANGPTL proteins, myelin inhibitors, and β-amyloid.

As used herein, the term “binding agent” or a grammatical equivalent thereof refers to a molecule (e.g., antibody) with one or more antigen-binding sites that binds an antigen. In some embodiments, a binding agent as described herein is an antibody (including a multispecific antibody and an antibody fragment, such as an antigen-binding fragment or an epitope-binding fragment) or other peptide-based molecule as well as a conjugate of an antibody, antibody fragment, or peptide-based molecule (e.g., an antibody-drug conjugate) that binds to LILRB1 (such as human LILRB1) and/or LILRB2 (such as human LILRB2).

The terms “antibody,” “immunoglobulin,” and “Ig” are used interchangeably herein, and are used in the broadest sense and specifically cover, for example polyclonal antibodies, monoclonal antibodies (including agonist, antagonist, neutralizing antibodies, full-length monoclonal antibodies), antibody compositions with polyepitopic or monoepitopic specificity, recombinantly produced antibodies, single domain (e.g., VHH) antibodies, monospecific antibodies, multispecific antibodies (including bispecific antibodies), synthetic antibodies, chimeric antibodies, humanized antibodies, or human versions of antibodies having full-length heavy and/or light chains. VHH as used herein refers to a domain antibody derived from a variable region of a heavy chain only antibody. Exemplary single domain antibodies include, but are not limited to, antibodies naturally devoid of light chains such as those from Camelidae species (e.g., llama), single domain antibodies derived from conventional 4-chain antibodies, engineered antibodies and single domain scaffolds other than those derived from antibodies. Single domain antibodies may be derived from any species including, but not limited to mouse, human, camel, llama, goat, rabbit, and bovine. VHH can also be derived from other species besides Camelidae that may produce heavy chain antibodies naturally devoid of light chain. Antibodies also include antibody fragments (and/or polypeptides that comprise antibody fragments) that retain LILRB1 and/or LILRB2 binding characteristics. Non-limiting examples of antibody fragments include antigen-binding regions and/or effector regions of the antibody, e.g., Fab, Fab′, F(ab′)₂, Fv, scFv, (scFv)₂, single chain antibody molecule, dual variable domain IgG (DVD-Ig), single variable domain, linear antibody, V region, a multispecific antibody formed from antibody fragments, F(ab)₂, Fd, Fc, diabody, di-diabody, disulfide-linked Fvs (dsFv), single-domain antibody (e.g., nanobody) or other fragments (e.g., fragments consisting of the variable regions of the heavy and light chains that are non-covalently coupled). In general terms, a variable (V) region domain may be any suitable arrangement of immunoglobulin heavy (VH) and/or light (VL) variable domains. For example, antibodies also include tetrameric antibodies comprising two heavy chain and two light chain molecules, an antibody light chain monomer, and an antibody heavy chain monomer. Thus, for example, the V region domain may be dimeric and contain VHH-VHH, VH-VH, VH-VL, or VL-VL dimers that bind LILRB1 and/or LILRB2. If desired, the VH and VL may be covalently coupled either directly or through a linker to form a single chain Fv (scFv). For ease of reference, scFv proteins are referred to herein as included in the category “antibody fragments.” Another form of an antibody fragment is a peptide comprising one or more complementarity determining regions (CDRs) of an antibody. CDRs (also termed “minimal recognition units” or “hypervariable regions”) can be obtained by constructing polynucleotides that encode one or more CDRs of interest. Such polynucleotides are prepared, for example, by using the polymerase chain reaction to synthesize the variable region using mRNA of antibody-producing cells as a template (see, for example, Larrick et al., Methods: A Companion to Methods in Enzymology, 2:106 (1991); Courtenay-Luck, “Genetic Manipulation of Monoclonal Antibodies,” in Monoclonal Antibodies Production, Engineering and Clinical Application, Ritter et al. (eds.), page 166, Cambridge University Press (1995); and Ward et al., “Genetic Manipulation and Expression of Antibodies,” in Monoclonal Antibodies: Principles and Applications, Birch et al., (eds.), page 137, Wiley-Liss, Inc. (1995)). Antibody fragments may be incorporated, for example, into single domain antibodies, maxibodies, minibodies, intrabodies, diabodies, triabodies, tetrabodies, variable domains of new antigen receptors (v-NAR), and bis-single chain Fv regions (see, e.g., Hollinger and Hudson, Nature Biotechnology, 23 (9): 1126-1136, 2005). In some embodiments, antibodies comprising a VH and/or VL contain a light chain and/or a heavy chain constant region, such as one or more constant regions, including one or more IgG1, IgG2, IgG3 and/or IgG4 constant regions. In some embodiments, antibodies can include epitope-binding fragments of any of the above. The antibodies described herein can be of any class (e.g., IgG, IgE, IgM, IgD, and IgA) or any subclass (e.g., IgG1, IgG2, IgG3, IgG4, IgA1, and IgA2) of immunoglobulin molecule.

The term “monospecific” when used in reference to a binding agent (e.g., an antibody) as used herein denotes a binding agent that has one or more binding sites each of which binds to the same epitope of the same antigen.

The term “multispecific” when used in reference to a binding agent (e.g., an antibody) means that the binding agent is able to specifically bind to at least two distinct epitopes, for example two binding sites each formed by a pair of an antibody heavy chain variable domain (VH) and an antibody light chain variable domain (VL) or each formed by a pair of VHH domains binding to different antigens or to different epitopes on the same antigen. Such a bispecific binding agent (e.g., an antibody) may have a 1+1 format (comprising one binding site for a first antigen or epitope and one binding site for a second antigen or epitope). Other bispecific binding agent (e.g., an antibody) formats may be 2+1 or 1+2 formats (comprising two binding sites for a first antigen or epitope and one binding site for a second antigen or epitope) or 2+2 format (comprising two binding sites for a first antigen or epitope and two binding sites for a second antigen or epitope). When a bispecific binding agent (e.g., an antibody) comprises two antigen-binding sites, each may bind to a different epitope. Such a bispecific binding agent (e.g., an antibody) may bind to two different epitopes on the same antigen (e.g., epitopes on LILRB1 or LILRB2).

The terms “identical” or percent “identity” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software that can be used to obtain alignments of amino acid or nucleotide sequences are well-known in the art. These include, but are not limited to, BLAST, ALIGN, Megalign, BestFit, GCG Wisconsin Package, and variants thereof. In some embodiments, two nucleic acids or polypeptides are substantially identical, meaning they have at least 70%, at least 75%, at least 80%, at least 85%, or at least 90%, and in some embodiments at least 95%, 96%, 97%, 98%, or 99% nucleotide or amino acid residue identity, when compared and aligned for maximum correspondence, as measured using a sequence comparison algorithm or by visual inspection. In some embodiments, identity exists over a region of the amino acid sequences that is at least about 10 residues, at least about 20 residues, at least about 40-60 residues, at least about 60-80 residues in length or any integral value there between. In some embodiments, identity exists over a longer region than 60-80 residues, such as at least about 80-100 residues, and in some embodiments the sequences are substantially identical over the full length of the sequences being compared, such as the coding region of a target protein or an antibody. In some embodiments, identity exists over a region of the nucleotide sequences that is at least about 10 bases, at least about 20 bases, at least about 40-60 bases, at least about 60-80 bases in length or any integral value there between. In some embodiments, identity exists over a longer region than 60-80 bases, such as at least about 80-1000 bases or more, and in some embodiments the sequences are substantially identical over the full-length of the sequences being compared, such as a nucleotide sequence encoding a protein of interest.

A “conservative amino acid substitution” is one in which one amino acid residue is replaced with another amino acid residue having a side chain with similar chemical characteristics. Families of amino acid residues having similar side chains have been generally defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. Generally, conservative substitutions in the sequences of the polypeptides, soluble proteins, and/or antibodies of the disclosure do not abrogate the binding of the polypeptide, soluble protein, or antibody containing the amino acid sequence, to the target binding site. Methods of identifying amino acid conservative substitutions which do not eliminate binding are well-known in the art.

As used herein, an “antigen” is a moiety or molecule that contains an epitope to which a binding agent (e.g., an antibody) can bind. As such, an antigen can be bound by an antibody. In some embodiments, the antigen, to which a binding agent (e.g., an antibody) described herein binds, is LILRB1 (e.g., human LILRB1), or a fragment thereof, including a fragment that comprises one or more domains of LILRB1. In some embodiments, the antigen, to which a binding agent (e.g., an antibody) described herein binds, is LILRB2 (e.g., human LILRB2), or a fragment thereof, including a fragment that comprises one or more domains of LILRB2. A target antigen may be a polypeptide, carbohydrate, nucleic acid, lipid, hapten, or other naturally occurring or synthetic compound. In some embodiments, the target antigen is a polypeptide. In certain embodiments, an antigen is associated with a cell, for example, is present on or in a cell.

As used herein, an “epitope” is a term in the art and refers to a localized region of an antigen to which a binding molecule (e.g., an antibody) can specifically bind. An epitope can be a linear epitope or a conformational, non-linear, or discontinuous epitope. In the case of a polypeptide antigen, for example, an epitope can be contiguous amino acids of the polypeptide (a “linear” epitope) or an epitope can comprise amino acids from two or more non-contiguous regions of the polypeptide (a “conformational,” “non-linear” or “discontinuous” epitope). It will be appreciated by one of skill in the art that, in general, a linear epitope may or may not be dependent on secondary, tertiary, or quaternary structure. For example, in some embodiments, a binding molecule binds to a group of amino acids regardless of whether they are folded in a natural three dimensional protein structure. In other embodiments, a binding molecule requires amino acid residues making up the epitope to exhibit a particular conformation (e.g., bend, twist, turn or fold) in order to recognize and bind the epitope.

An antibody binds “an epitope” or “essentially the same epitope” or “the same epitope” as a reference antibody, when the two antibodies recognize identical, overlapping or adjacent epitopes in a three-dimensional space. The most widely used and rapid methods for determining whether two antibodies bind to identical, overlapping or adjacent epitopes in a three-dimensional space are competition assays, which can be configured in a number of different formats, for example, using either labeled antigen or labeled antibody. In some assays, the antigen is immobilized on a 96-well plate, or expressed on a cell surface, and the ability of unlabeled antibodies to block the binding of labeled antibodies is measured using radioactive, fluorescent or enzyme labels.

“Epitope binning” is the process of grouping antibodies based on the epitopes they recognize. More particularly, epitope binning comprises methods and systems for discriminating the epitope recognition properties of different antibodies, using competition assays combined with computational processes for clustering antibodies based on their epitope recognition properties and identifying antibodies having distinct binding specificities.

An “intact” antibody is one comprising an antigen-binding site as well as a CL and at least heavy chain constant regions, CH1, CH2 and CH3. The constant regions may include human constant regions or amino acid sequence variants thereof. In certain embodiments, an intact antibody has one or more effector functions.

The terms “binds” or “binding” refer to an interaction between molecules including, for example, to form a complex. Interactions can be, for example, non-covalent interactions including hydrogen bonds, ionic bonds, hydrophobic interactions, and/or van der Waals interactions. A complex can also include the binding of two or more molecules held together by covalent or non-covalent bonds, interactions, or forces. The strength of the total non-covalent interactions between a single antigen-binding site on an antibody and a single epitope of a target molecule, such as an antigen, is the affinity of the antibody or functional fragment for that epitope. The ratio of dissociation rate (k_{off}) to association rate (kₒₙ) of a binding molecule (e.g., an antibody) to a monovalent antigen (k_{off}/kₒₙ) is the dissociation constant K_{D}, which is inversely related to affinity. The lower the K_{D} value, the higher the affinity of the antibody. The value of K_{D} varies for different complexes of antibody and antigen and depends on both kₒₙ and k_{off}. The dissociation constant K_{D} for an antibody provided herein can be determined using any method provided herein or any other method well known to those skilled in the art. The affinity at one binding site does not always reflect the true strength of the interaction between an antibody and an antigen. When complex antigens containing multiple, repeating antigenic determinants, such as a polyvalent antigen, come in contact with antibodies containing multiple binding sites, the interaction of antibody with antigen at one site will increase the probability of a reaction at a second site. The strength of such multiple interactions between a multivalent antibody and antigen is called the avidity.

In connection with the binding molecules described herein terms such as “bind to,” “that specifically bind to,” and analogous terms are also used interchangeably herein and refer to binding molecules of antigen binding domains that specifically bind to an antigen, such as a polypeptide. A binding molecule or antigen binding domain that binds to or specifically binds to an antigen can be identified, for example, by immunoassays, Octet®, Biacore®, or other techniques known to those of skill in the art. In some embodiments, a binding molecule or antigen binding domain binds to or specifically binds to an antigen when it binds to an antigen with higher affinity than to any cross-reactive antigen as determined using experimental techniques, such as enzyme linked immunosorbent assay (ELISA). Typically, a specific or selective reaction will be at least twice background signal or noise and may be more than 10 times background. See, e.g., Fundamental Immunology 332-36 (Paul ed., 2d ed. 1989) for a discussion regarding binding specificity. In certain embodiments, the extent of binding of a binding molecule or antigen binding domain to a “non-target” protein is less than about 10% of the binding of the binding molecule or antigen binding domain to its particular target antigen, for example, as determined by fluorescence activated cell sorting (FACS) analysis. A binding molecule or antigen binding domain that binds to an antigen includes one that is capable of binding the antigen with sufficient affinity such that the binding molecule is useful, for example, as a therapeutic and/or diagnostic agent in targeting the antigen. In certain embodiments, a binding molecule or antigen binding domain that binds to an antigen has a dissociation constant (K_{D}) of less than or equal to 1 μM, 800 nM, 600 nM, 550 nM, 500 nM, 300 nM, 250 nM, 100 nM, 50 nM, 10 nM, 5 nM, 4 nM, 3 nM, 2 nM, 1 nM, 0.9 nM, 0.8 nM, 0.7 nM, 0.6 nM, 0.5 nM, 0.4 nM, 0.3 nM, 0.2 nM, or 0.1 nM. In certain embodiments, a binding molecule or antigen binding domain binds to an epitope of an antigen that is conserved among the antigen from different species.

“Binding affinity” generally refers to the strength of the sum total of noncovalent interactions between a single binding site of a molecule (e.g., a binding agent such as an antibody) and its binding partner (e.g., antigen such as LILRB1 and/or LILRB2). Unless indicated otherwise, as used herein, “binding affinity” refers to intrinsic binding affinity which reflects a 1:1 interaction between members of a binding pair (e.g., antibody and antigen). The affinity of a binding molecule X for its binding partner Y can generally be represented by the dissociation constant (K_{D}). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present disclosure. In one embodiment, the “K_{D}” or “K_{D} value” may be measured by biolayer interferometry (BLI) using, for example, the OctetQK384 system (ForteBio, Menlo Park, CA). Alternatively, the K_{D} may also be measured in a radiolabeled antigen-binding assay (RIA), for example, performed with the Fab version of an antibody of interest and its antigen (Chen, et al., (1999) J. Mol Biol 293:865-881) or using surface plasmon resonance (SPR) assays by BIACORE™, using, for example, a BIACORE™-2000 or a BIACORE™-3000 (BIACORE™, Inc., Piscataway, NJ). An “on-rate” or “rate of association” or “association rate” or “kₒₙ,” as well as an “off-rate” or “rate of dissociation” or “dissociation rate” or “k_{off},” can also be determined with the same SPR or BLI techniques described above using, for example, the OctetQK384 system (ForteBio, Menlo Park, CA) or a BIACORE™ 2000 or a BIACORE™-3000 (BIACORE™, Inc., Piscataway, NJ), respectively.

The term “compete” when used in the context of binding agents (e.g., antibodies) means binding agents that compete for the same epitope or binding site on a target, which includes competition between such binding agents as determined by an assay in which the binding agent under study prevents or inhibits the specific binding of a reference molecule (e.g., a reference ligand, or reference antigen-binding protein, such as a reference antibody) to a common antigen (e.g., LILRB1 and/or LILRB2). Numerous types of competitive binding assays can be used to determine if a test binding agent competes with a reference molecule for binding to LILRB1 (e.g., human LILRB1) or LILRB2 (e.g., human LILRB2). Examples of assays that can be employed include solid phase direct or indirect radioimmunoassay (RIA); solid phase direct or indirect enzyme immunoassay (EIA), sandwich competition assay (see, e.g., Stahli et al., (1983) Methods in Enzymology 9:242-253); solid phase direct biotin-avidin EIA (see, e.g., Kirkland et al., (1986) J. Immunol. 137:3614-3619 or Cheung, et al., (1990) Virology 176:546-552); solid phase direct labeled assay; solid phase direct labeled sandwich assay (see, e.g., Harlow and Lane, (1988) Antibodies, A Laboratory Manual, Cold Spring Harbor Press); solid phase direct label RIA using I-125 label (see, e.g., Morel et al., (1988) Molec. Immunol. 25:7-15); and direct labeled RIA (Moldenhauer et al., (1990) Scand. J. Immunol. 32:77-82). Typically, such an assay involves the use of a purified antigen (e.g., LILRB1, such as human LILRB1, or LILRB2, such as human LILRB2) bound to a solid surface or cells bearing either of an unlabeled test antigen-binding protein (e.g., test LILRB1 antibody or test LILRB2 antibody) or a labeled reference antigen-binding protein (e.g., reference LILRB1 antibody or reference LILRB2 antibody). Competitive inhibition may be measured by determining the amount of label bound to the solid surface or cells in the presence of the test antigen-binding protein. Usually, the test antigen-binding protein is present in excess. Antibodies identified by competition assay (competing antibodies) include antibodies binding to the same epitope as the reference antibody and/or antibodies binding to an adjacent epitope sufficiently proximal to the epitope bound by the reference for antibodies steric hindrance to occur (e.g., similar epitope or overlapping epitope). Usually, when a competing antibody is present in excess, it will inhibit specific binding of a reference antibody to a common antigen by at least 20%, for example, at least 20%, 25%, 30%, 35%, 40%, 45%, 50%, 55%, 60%, 65%, 70% or 75%. In some instance, binding is inhibited by at least 80%, 85%, 90%, 95%, 96%, 97%, 98%, 99% or more.

Antibody “effector functions” refer to those biological activities attributable to the Fc region (e.g., a native sequence Fc region or amino acid sequence variant Fc region) of an antibody, and vary with the antibody isotype. Examples of antibody effector functions include: C1q binding and complement dependent cytotoxicity; Fc receptor binding; antibody-dependent cell-mediated cytotoxicity (ADCC); phagocytosis; down regulation of cell surface receptors (e.g., B cell receptor); and B cell activation.

The term “Fc region” herein is used to define a C-terminal region of an immunoglobulin heavy chain, including, for example, native sequence Fc regions, recombinant Fc regions, and variant Fc regions. Although the boundaries of the Fc region of an immunoglobulin heavy chain might vary, the human IgG heavy chain Fc region is often defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. A “functional Fc region” possesses an “effector function” of a native sequence Fc region. Exemplary “effector functions” include C1q binding; CDC; Fc receptor binding; ADCC; phagocytosis; downregulation of cell surface receptors (e.g., B cell receptor), etc. Such effector functions generally require the Fc region to be combined with a binding region or binding domain (e.g., an antibody variable region or domain) and can be assessed using various assays known to those skilled in the art. A “native sequence Fc region” comprises an amino acid sequence identical to the amino acid sequence of an Fc region found in nature, and not manipulated, modified, and/or changed (e.g., isolated, purified, selected, including or combining with other sequences such as variable region sequences) by a human. Native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof. A “variant Fc region” comprises an amino acid sequence which differs from that of a native sequence Fc region by virtue of at least one amino acid modification (e.g., substituting, addition, or deletion). In certain embodiments, the variant Fc region has at least one amino acid substitution compared to a native sequence Fc region or to the Fc region of a parent polypeptide, for example, from about one to about ten amino acid substitutions, or from about one to about five amino acid substitutions in a native sequence Fc region or in the Fc region of a parent polypeptide. The variant Fc region herein can possess at least about 80% homology with a native sequence Fc region and/or with an Fc region of a parent polypeptide, or at least about 90% homology therewith, for example, at least about 95% homology therewith.

In certain embodiments, the binding molecules or antigen binding domains can comprise “chimeric” sequences in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see U.S. Pat. No. 4,816,567; and Morrison et al., 1984, Proc. Natl. Acad. Sci. USA 81:6851-55). Chimeric sequences may include humanized sequences.

In certain embodiments, the binding molecules or antigen binding domains can comprise portions of “humanized” forms of nonhuman (e.g., camelid, murine, non-human primate) antibodies that include sequences from human immunoglobulins (e.g., recipient antibody) in which the native CDR residues are replaced by residues from the corresponding CDR of a nonhuman species (e.g., donor antibody) such as camelid, mouse, rat, rabbit, or nonhuman primate having the desired specificity, affinity, and capacity. In some instances, one or more FR region residues of the human immunoglobulin sequences are replaced by corresponding nonhuman residues. Furthermore, humanized antibodies can comprise residues that are not found in the recipient antibody or in the donor antibody. These modifications are made to further refine antibody performance. A humanized antibody heavy or light chain can comprise substantially all of at least one or more variable regions, in which all or substantially all of the CDRs correspond to those of a nonhuman immunoglobulin and all or substantially all of the FRs are those of a human immunoglobulin sequence. In certain embodiments, the humanized antibody will comprise at least a portion of an immunoglobulin constant region (Fc), typically that of a human immunoglobulin. For further details, see, Jones et al., Nature 321:522-25 (1986); Riechmann et al., Nature 332:323-29 (1988); Presta, Curr. Op. Struct. Biol. 2:593-96 (1992); Carter et al., Proc. Natl. Acad. Sci. USA 89:4285-89 (1992); U.S. Pat. Nos. 6,800,738; 6,719,971; 6,639,055; 6,407,213; and 6,054,297.

In certain embodiments, the binding molecules or antigen binding domains can comprise portions of a “fully human antibody” or “human antibody,” wherein the terms are used interchangeably herein and refer to an antibody that comprises a human variable region and, for example, a human constant region. The binding molecules may comprise an antibody sequence. In specific embodiments, the terms refer to an antibody that comprises a variable region and constant region of human origin. “Fully human” antibodies, in certain embodiments, can also encompass antibodies which bind polypeptides and are encoded by nucleic acid sequences which are naturally occurring somatic variants of human germline immunoglobulin nucleic acid sequence. The term “fully human antibody” includes antibodies having variable and constant regions corresponding to human germline immunoglobulin sequences as described by Kabat et al. (See Kabat et al. (1991) Sequences of Proteins of Immunological Interest, Fifth Edition, U.S. Department of Health and Human Services, NIH Publication No. 91-3242). A “human antibody” is one that possesses an amino acid sequence which corresponds to that of an antibody produced by a human and/or has been made using any of the techniques for making human antibodies. This definition of a human antibody specifically excludes a humanized antibody comprising non-human antigen-binding residues. Human antibodies can be produced using various techniques known in the art, including phage-display libraries (Hoogenboom and Winter, J. Mol. Biol. 227:381 (1991); Marks et al., J. Mol. Biol. 222:581 (1991)) and yeast display libraries (Chao et al., Nature Protocols 1:755-68 (2006)). Also available for the preparation of human monoclonal antibodies are methods described in Cole et al., Monoclonal Antibodies and Cancer Therapy 77 (1985); Boerner et al., J. Immunol. 147 (1): 86-95 (1991); and van Dijk and van de Winkel, Curr. Opin. Pharmacol. 5:368-74 (2001). Human antibodies can be prepared by administering the antigen to a transgenic animal that has been modified to produce such antibodies in response to antigenic challenge, but whose endogenous loci have been disabled, e.g., mice (see, e.g., Jakobovits, Curr. Opin. Biotechnol. 6 (5): 561-66 (1995); Brüggemann and Taussing, Curr. Opin. Biotechnol. 8 (4): 455-58 (1997); and U.S. Pat. Nos. 6,075,181 and 6,150,584 regarding XENOMOUSE™ technology). See also, for example, Li et al., Proc. Natl. Acad. Sci. USA 103:3557-62 (2006) regarding human antibodies generated via a human B-cell hybridoma technology.

In certain embodiments, the binding molecules or antigen binding domains can comprise portions of a “recombinant human antibody,” wherein the phrase includes human antibodies that are prepared, expressed, created or isolated by recombinant means, such as antibodies expressed using a recombinant expression vector transfected into a host cell, antibodies isolated from a recombinant, combinatorial human antibody library, antibodies isolated from an animal (e.g., a mouse or cow) that is transgenic and/or transchromosomal for human immunoglobulin genes (see, e.g., Taylor, L. D. et al., Nucl. Acids Res. 20:6287-6295 (1992)) or antibodies prepared, expressed, created, or isolated by any other means that involves splicing of human immunoglobulin gene sequences to other DNA sequences. Such recombinant human antibodies can have variable and constant regions derived from human germline immunoglobulin sequences (See Kabat, E. A. et al. (1991) Sequences of Proteins of Immunological Interest, Fifth Edition, U.S. Department of Health and Human Services, NIH Publication No. 91-3242). In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

In certain embodiments, the binding molecules or antigen binding domains can comprise a portion of a “monoclonal antibody,” wherein the term as used herein refers to an antibody obtained from a population of substantially homogeneous antibodies, e.g., the individual antibodies comprising the population are identical except for possible naturally occurring mutations that may be present in minor amounts or well-known post-translational modifications such as amino acid isomerization or deamidation, methionine oxidation or asparagine or glutamine deamidation, each monoclonal antibody will typically recognize a single epitope on the antigen. In specific embodiments, a “monoclonal antibody,” as used herein, is an antibody produced by a single hybridoma or other cell. The term “monoclonal” is not limited to any particular method for making the antibody. For example, the monoclonal antibodies useful in the present disclosure may be prepared by the hybridoma methodology first described by Kohler et al., Nature 256:495 (1975), or may be made using recombinant DNA methods in bacterial or eukaryotic animal or plant cells (see, e.g., U.S. Pat. No. 4,816,567). The “monoclonal antibodies” may also be isolated from phage antibody libraries using the techniques described in Clackson et al., Nature 352:624-28 (1991) and Marks et al., J. Mol. Biol. 222:581-97 (1991), for example. Other methods for the preparation of clonal cell lines and of monoclonal antibodies expressed thereby are well known in the art. See, e.g., Short Protocols in Molecular Biology (Ausubel et al. eds., 5th ed. 2002).

The terms “antigen-binding fragment,” “antigen-binding domain,” “antigen-binding region,” and similar terms refer to that portion of an antibody, which comprises the amino acid residues that interact with an antigen and confer on the binding fragment, domain, or region its specificity and affinity for the antigen (e.g., the CDRs). “Antigen-binding fragment” as used herein includes “antibody fragment,” which comprises a portion of an antibody including one or more CDRs, such as the antigen-binding or variable region of the antibody.

Antibodies described herein include, but are not limited to, synthetic antibodies, monoclonal antibodies, recombinantly produced antibodies, multispecific antibodies (e.g., bispecific antibodies), human antibodies, humanized antibodies, chimeric antibodies, intrabodies, single-chain Fvs (scFv) (e.g., including monospecific, bispecific, etc.), camelized antibodies, Fab fragments, F(ab′) fragments, disulfide-linked Fvs (sdFv), anti-idiotypic (anti-Id) antibodies, and epitope-binding fragments of any of the above.

In some embodiments, antibodies described herein include immunoglobulin molecules and immunologically active portions of immunoglobulin molecules, including molecules that contain one or more antigen-binding sites that bind to a LILRB1 antigen and/or a LILRB2 antigen.

Antibodies can be of any type (e.g., IgG, IgE, IgM, IgD, IgA or IgY), any class (e.g., IgG1, IgG2, IgG3, IgG4, IgA1 or IgA2), or any subclass (e.g., IgG2a or IgG2b) of immunoglobulin molecule. In some embodiments, antibodies described herein are IgG antibodies (e.g., human IgG), or a class (e.g., human IgG1, IgG2, IgG3 or IgG4) or a subclass thereof.

A typical 4-chain antibody unit is a heterotetrametric glycoprotein composed of two identical light (L) chains and two identical heavy (H) chains. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (VH) followed by three constant domains (CH) for each of the α and γ chains and four CH domains for u and & isotypes. Each L chain has at the N-terminus, a variable domain (VL) followed by a constant domain (CL) at its other end. The VL is aligned with the VH, and the CL is aligned with the first constant domain of the heavy chain (CH1). Particular amino acid residues are believed to form an interface between the light chain and heavy chain variable domains. The pairing of a VH and VL together forms a single antigen-binding site. For the structure and properties of the different classes of antibodies, see, for example, Basic and Clinical Immunology 71 (Stites et al. eds., 8th ed. 1994); and Immunobiology (Janeway et al. eds., 5th ed. 2001).

In some embodiments, an antibody is a 4-chain antibody unit comprising two heavy (H) chain/light (L) chain pairs. In further embodiments, the amino acid sequences of the H chains are identical and the amino acid sequences of the L chains are identical. In other embodiments, the amino acid sequences of the H chains are different from each other. Additionally or alternatively, the amino acid sequences of the L chains are different from each other. For example, an antibody comprises a first H/L chain pair and a second H/L chain pair, wherein the first H/L chain pair binds to an LILRB1 antigen and the second H/L chain pair binds to a non-LILRB1 (such as LILRB2) antigen. In some embodiments, an antibody is a 2-chain antibody unit comprising a VHH-VHH pair. In further embodiments, the amino acid sequences of the VHH are identical. In other embodiments, the amino acid sequence of the VHH are different from each other. For example, an antibody comprises a first VHH and a second VHH, wherein the first VHH binds to an LILRB1 antigen and the second VHH binds to a non-LILRB1 (such as LILRB2) antigen. In some embodiments, the H and/or L chains comprise constant regions, for example, human constant regions. In some embodiments, the L chain constant region of such antibodies is a kappa or lambda light chain constant region, for example, a human kappa or lambda light chain constant region. In some embodiments, the H chain constant region of such antibodies comprises a gamma heavy chain constant region, for example, a human gamma heavy chain constant region. In some embodiments, such antibodies comprise IgG constant regions, for example, human IgG constant regions (e.g., IgG1, IgG2, IgG3, and/or IgG4 constant regions).

An antibody or fragment thereof may preferentially bind to LILRB1 (such as human LILRB1) and/or LILRB2 (such as human LILRB2), meaning that the antibody or fragment thereof binds LILRB1 and/or LILRB2 with greater affinity than it binds to a control protein (e.g., unrelated control proteins such as hen egg white lysozyme) and/or binds human LILRB1 and/or LILRB2 with greater affinity than it binds to an unrelated control protein. For example, the antibody or fragment thereof may specifically recognize and bind LILRB1 and/or LILRB2, or a portion of each thereof. “Specific binding” means that the antibody or fragment thereof binds to LILRB1 and/or LILRB2 with an affinity that is at least 5, 10, 15, 20, 25, 50, 100, 250, 500, 1000, or 10,000 times greater than the affinity for an unrelated control protein (e.g., hen egg white lysozyme). In some embodiments, the antibody or fragment thereof may bind LILRB1 and/or LILRB2 substantially exclusively (e.g., is able to distinguish LILRB1 and/or LILRB2 from other known polypeptides, for example, by virtue of measurable differences in binding affinity). In some embodiments, an LILRB1 binding agent (e.g., an antibody) may react with LILRB1 sequences other than human LILRB1 sequences (e.g., cynomolgous money LILRB1 sequences). In other embodiments, an LILRB1 binding agent (e.g., an antibody) does not react with non-human (such as cynomolgous money LILRB1 sequences). In some embodiments, a LILRB2 binding agent (e.g., an antibody) may react with LILRB2 sequences other than human LILRB2 sequences (e.g., cynomolgous monkey LILRB2 sequences). In other embodiments, a LILRB2 binding agent (e.g., an antibody) does not react with non-human (such as cynomolgous monkey LILRB2 sequences).

The term “Fab” or “Fab region” refers to an antibody region that binds to antigens. A conventional IgG usually comprises two Fab regions, each residing on one of the two arms of the Y-shaped IgG structure. Each Fab region is typically composed of one variable region and one constant region of each of the heavy and the light chain. More specifically, the variable region and the constant region of the heavy chain in a Fab region are VH and CHI regions, and the variable region and the constant region of the light chain in a Fab region are VL and CL regions. The VH, CHI, VL, and CL in a Fab region can be arranged in various ways to confer an antigen binding capability according to the present disclosure. For example, VH and CHI regions can be on one polypeptide, and VL and CL regions can be on a separate polypeptide, similarly to a Fab region of a conventional IgG. Alternatively, VH, CH1, VL and CL regions can all be on the same polypeptide and oriented in different orders as described in more detail the sections below.

The term “variable region,” “variable domain,” “V region,” or “V domain” refers to a portion of the light or heavy chains of an antibody that is generally located at the amino-terminal of the light or heavy chain and has a length of about 120 to 130 amino acids in the heavy chain and about 100 to 110 amino acids in the light chain, and are used in the binding and specificity of each particular antibody for its particular antigen. The variable region of the heavy chain may be referred to as “VH.” The variable region of the light chain may be referred to as “VL.” The term “variable” refers to the fact that certain segments of the variable regions differ extensively in sequence among antibodies. The V region mediates antigen binding and defines specificity of a particular antibody for its particular antigen. However, the variability is not evenly distributed across the 110-amino acid span of the variable regions. Instead, the V regions consist of less variable (e.g., relatively invariant) stretches called framework regions (FRs) of about 15-30 amino acids separated by shorter regions of greater variability (e.g., extreme variability) called “hypervariable regions” that are each about 9-12 amino acids long. The variable regions of heavy and light chains each comprise four FRs, largely adopting a β sheet configuration, connected by three hypervariable regions, which form loops connecting, and in some cases form part of, the β sheet structure. The hypervariable regions in each chain are held together in close proximity by the FRs and, with the hypervariable regions from the other chain, contribute to the formation of the antigen-binding site of antibodies (see, e.g., Kabat et al., Sequences of Proteins of Immunological Interest (5th ed. 1991)). The constant regions are not involved directly in binding an antibody to an antigen, but exhibit various effector functions, such as participation of the antibody in antibody dependent cellular cytotoxicity (ADCC) and complement dependent cytotoxicity (CDC). The variable regions differ extensively in sequence between different antibodies. In specific embodiments, the variable region is a human variable region.

The term “variable region residue numbering according to Kabat” or “amino acid position numbering as in Kabat”, and variations thereof, refer to the numbering system used for heavy chain variable regions or light chain variable regions of the compilation of antibodies in Kabat et al., supra. Using this numbering system, the actual linear amino acid sequence may contain fewer or additional amino acids corresponding to a shortening of, or insertion into, an FR or CDR of the variable domain. For example, a heavy chain variable domain may include a single amino acid insert (residue 52a according to Kabat) after residue 52 and three inserted residues (e.g., residues 82a, 82b, and 82c, etc. according to Kabat) after residue 82. The Kabat numbering of residues may be determined for a given antibody by alignment at regions of homology of the sequence of the antibody with a “standard” Kabat numbered sequence. The Kabat numbering system is generally used when referring to a residue in the variable domain (approximately residues 1-107 of the light chain and residues 1-113 of the heavy chain) (e.g., Kabat et al., supra). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG 1 EU antibody. Other numbering systems have been described, for example, by AbM, Chothia, Contact, IMGT, and AHon.

As used herein, the term “heavy chain” when used in reference to an antibody refers to a polypeptide chain of about 50-70 kDa, wherein the amino-terminal portion includes a variable region of about 120 to 130 or more amino acids, and a carboxy-terminal portion includes a constant region. The constant region can be one of five distinct types, (e.g., isotypes) referred to as alpha (α), delta (δ), epsilon (ε), gamma (γ), and mu (μ), based on the amino acid sequence of the heavy chain constant region. The distinct heavy chains differ in size: α, δ, and γ contain approximately 450 amino acids, while μ and ε contain approximately 550 amino acids. When combined with a light chain, these distinct types of heavy chains give rise to five well known classes (e.g., isotypes) of antibodies, IgA, IgD, IgE, IgG, and IgM, respectively, including four subclasses of IgG, namely IgG1, IgG2, IgG3, and IgG4.

As used herein, the term “light chain” when used in reference to an antibody refers to a polypeptide chain of about 25 kDa, wherein the amino-terminal portion includes a variable region of about 100 to about 110 or more amino acids, and a carboxy-terminal portion includes a constant region. The approximate length of a light chain is 211 to 217 amino acids. There are two distinct types, referred to as kappa (κ) or lambda (λ) based on the amino acid sequence of the constant domains.

As used herein, the terms “hypervariable region,” “HVR,” “Complementarity Determining Region,” and “CDR” are used interchangeably. A “CDR” refers to one of three hypervariable regions (H1, H2 or H3) within the non-framework region of the immunoglobulin (Ig or antibody) VH β-sheet framework, or one of three hypervariable regions (L1, L2 or L3) within the non-framework region of the antibody VL β-sheet framework. CDR1, CDR2 and CDR3 in VH domain are also referred to as HCDR1, HCDR2 and HCDR3, respectively. CDR1, CDR2 and CDR3 in VL domain are also referred to as LCDR1, LCDR2 and LCDR3, respectively. Accordingly, CDRs are variable region sequences interspersed within the framework region sequences.

CDR regions are well known to those skilled in the art and have been defined by well-known numbering systems. For example, the Kabat Complementarity Determining Regions (CDRs) are based on sequence variability and are the most commonly used (see, e.g., Kabat et al., supra; Nick Deschacht et al., J Immunol 2010; 184:5696-5704). Chothia refers instead to the location of the structural loops (see, e.g., Chothia and Lesk, J. Mol. Biol. 196:901-17 (1987)). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software (see, e.g., Antibody Engineering Vol. 2 (Kontermann and Dübel eds., 2d ed. 2010)). The “contact” hypervariable regions are based on an analysis of the available complex crystal structures. Another universal numbering system that has been developed and widely adopted is ImMunoGeneTics (IMGT) Information System® (Lafranc et al., Dev. Comp. Immunol. 27 (1): 55-77 (2003)). IMGT is an integrated information system specializing in immunoglobulins (IG), T-cell receptors (TCR), and major histocompatibility complex (MHC) of human and other vertebrates. Herein, the CDRs are referred to in terms of both the amino acid sequence and the location within the light or heavy chain. As the “location” of the CDRs within the structure of the immunoglobulin variable domain is conserved between species and present in structures called loops, by using numbering systems that align variable domain sequences according to structural features, CDR and framework residues are readily identified. This information can be used in grafting and replacement of CDR residues from immunoglobulins of one species into an acceptor framework from, typically, a human antibody. An additional numbering system (AHon) has been developed by Honegger and Plückthun, J. Mol. Biol. 309:657-70 (2001). Correspondence between the numbering system, including, for example, the Kabat numbering and the IMGT unique numbering system, is well known to one skilled in the art (see, e.g., Kabat, supra; Chothia and Lesk, supra; Martin, supra; Lefranc et al., supra). The residues from each of these hypervariable regions or CDRs are exemplified in the Table below.

Exemplary CDRs According to Various Numbering Systems

| Loop | Kabat | AbM | Chothia | Contact | IMGT |
|---|---|---|---|---|---|
| CDR L1 | L24—L34 | L24—L34 | L26—L32 or | L30—L36 | L27—L38 |
| | | | L24—L34 | | |
| CDR L2 | L50—L56 | L50—L56 | L50—L52 or | L46—L55 | L56—L65 |
| | | | L50—L56 | | |
| CDR L3 | L89—L97 | L89—L97 | L91—L96 or | L89—L96 | L105—L117 |
| | | | L89—L97 | | |
| CDR H1 | H31—H35B | H26—H35B | H26—H32 . . . 34 | H30—H35B | H27—H38 |
| | (Kabat | | | | |
| | Numbering) | | | | |
| CDR H1 | H31—H35 | H26—H35 | H26—H32 | H30—H35 | |
| | (Chothia | | | | |
| | Numbering) | | | | |
| CDR H2 | H50—H65 | H50—H58 | H53—H55 or | H47—H58 | H56—H65 |
| | | | H52—H56 | | |
| CDR H3 | H95—H102 | H95—H102 | H96—H101 or | H93—H101 | H105—H117 |
| | | | H95—H102 | | |

The boundaries of a given CDR may vary depending on the scheme used for identification. Thus, unless otherwise specified, the terms “CDR” and “complementary determining region” of a given antibody or region thereof, such as a variable region, as well as individual CDRs (e.g., CDR-H1, CDR-H2) of the antibody or region thereof, should be understood to encompass the complementary determining region as defined by any of the known schemes described herein above. In some instances, the scheme for identification of a particular CDR or CDRs is specified, such as the CDR as defined by the IMGT, Kabat, Chothia, or Contact method. In other cases, the particular amino acid sequence of a CDR is given. It should be noted CDR regions may also be defined by a combination of various numbering systems, e.g., a combination of Kabat and Chothia numbering systems, or a combination of Kabat and IMGT numbering systems. Therefore, the term such as “a CDR1 as set forth in a specific VH” includes any CDR1 as defined by the exemplary CDR numbering systems described above, but is not limited thereby. Once a variable region (e.g., a VH or VL) is given, those skilled in the art would understand that CDRs within the region can be defined by different numbering systems or combinations thereof.

Hypervariable regions may comprise “extended hypervariable regions” as follows: 24-36 or 24-34 (L1), 46-56 or 50-56 (L2), and 89-97 or 89-96 (L3) in the VL, and 26-35 or 26-35A (H1), 50-65 or 49-65 (H2), and 93-102, 94-102, or 95-102 (H3) in the VH.

The term “constant region” or “constant domain” refers to a carboxy terminal portion of the light and heavy chain which is not directly involved in binding of the antibody to antigen but exhibits various effector function, such as interaction with the Fc receptor. The term refers to the portion of an immunoglobulin molecule having a more conserved amino acid sequence relative to the other portion of the immunoglobulin, the variable region, which contains the antigen binding site. The constant region may contain the CH1, CH2, and CH3 regions of the heavy chain and the CL region of the light chain.

The term “framework” or “FR” refers to those variable region residues flanking the CDRs. FR residues are present, for example, in chimeric, humanized, human, domain antibodies, diabodies, linear antibodies, and bispecific antibodies. FR residues are those variable domain residues other than the hypervariable region residues or CDR residues.

“Percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared.

The term “specificity” refers to selective recognition of an antigen binding protein for a particular epitope of an antigen. Natural antibodies, for example, are monospecific. The term “multispecific” as used herein denotes that an antigen binding protein has two or more antigen-binding sites of which at least two bind different antigens. “Bispecific” as used herein denotes that an antigen binding protein has two different antigen-binding specificities. The term “monospecific” antibody as used herein denotes an antigen binding protein that has one or more binding sites each of which bind the same antigen.

The term “valent” as used herein denotes the presence of a specified number of binding sites in an antigen binding protein. A natural antibody for example or a full length antibody has two binding sites and is bivalent. As such, the terms “trivalent”, “tetravalent”, “pentavalent” and “hexavalent” denote the presence of two binding site, three binding sites, four binding sites, five binding sites, and six binding sites, respectively, in an antigen binding protein.

The terms “polypeptide” and “peptide” and “protein” are used interchangeably herein and refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification. Also included within the definition are, for example, polypeptides containing one or more analogs of an amino acid, including but not limited to, unnatural amino acids, as well as other modifications known in the art. It is understood that, because the polypeptides of this disclosure may be based upon antibodies or other members of the immunoglobulin superfamily, in certain embodiments, a “polypeptide” can occur as a single chain or as two or more associated chains.

“Polynucleotide” or “nucleic acid,” as used interchangeably herein, refers to polymers of nucleotides of any length and includes DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs. “Oligonucleotide,” as used herein, refers to short, generally single-stranded, synthetic polynucleotides that are generally, but not necessarily, fewer than about 200 nucleotides in length. The terms “oligonucleotide” and “polynucleotide” are not mutually exclusive. The description above for polynucleotides is equally and fully applicable to oligonucleotides. A cell that produces a binding molecule of the present disclosure may include a parent hybridoma cell, as well as bacterial and eukaryotic host cells into which nucleic acids encoding the antibodies have been introduced. Unless specified otherwise, the left-hand end of any single-stranded polynucleotide sequence disclosed herein is the 5′ end; the left-hand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA transcript that are 5′ to the 5′ end of the RNA transcript are referred to as “upstream sequences”; sequence regions on the DNA strand having the same sequence as the RNA transcript that are 3′ to the 3′ end of the RNA transcript are referred to as “downstream sequences.”

An “isolated nucleic acid” is a nucleic acid, for example, an RNA, DNA, or a mixed nucleic acids, which is substantially separated from other genome DNA sequences as well as proteins or complexes such as ribosomes and polymerases, which naturally accompany a native sequence. An “isolated” nucleic acid molecule is one which is separated from other nucleic acid molecules which are present in the natural source of the nucleic acid molecule. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. In a specific embodiment, one or more nucleic acid molecules encoding an antibody as described herein are isolated or purified. The term embraces nucleic acid sequences that have been removed from their naturally occurring environment, and includes recombinant or cloned DNA isolates and chemically synthesized analogues or analogues biologically synthesized by heterologous systems. A substantially pure molecule may include isolated forms of the molecule. Specifically, an “isolated” nucleic acid molecule encoding an antibody described herein is a nucleic acid molecule that is identified and separated from at least one contaminant nucleic acid molecule with which it is ordinarily associated in the environment in which it was produced.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

As used herein, the term “operatively linked,” and similar phrases (e.g., genetically fused), when used in reference to nucleic acids or amino acids, refer to the operational linkage of nucleic acid sequences or amino acid sequence, respectively, placed in functional relationships with each other. For example, an operatively linked promoter, enhancer elements, open reading frame, 5′ and 3′ UTR, and terminator sequences result in the accurate production of a nucleic acid molecule (e.g., RNA). In some embodiments, operatively linked nucleic acid elements result in the transcription of an open reading frame and ultimately the production of a polypeptide (i.e., expression of the open reading frame). As another example, an operatively linked peptide is one in which the functional domains are placed with appropriate distance from each other to impart the intended function of each domain.

The term “vector” refers to a substance that is used to carry or include a nucleic acid sequence, including for example, a nucleic acid sequence encoding a binding molecule (e.g., an antibody) as described herein, in order to introduce a nucleic acid sequence into a host cell. Vectors applicable for use include, for example, expression vectors, plasmids, phage vectors, viral vectors, episomes, and artificial chromosomes, which can include selection sequences or markers operable for stable integration into a host cell's chromosome. Additionally, the vectors can include one or more selectable marker genes and appropriate expression control sequences. Selectable marker genes that can be included, for example, provide resistance to antibiotics or toxins, complement auxotrophic deficiencies, or supply critical nutrients not in the culture media. Expression control sequences can include constitutive and inducible promoters, transcription enhancers, transcription terminators, and the like, which are well known in the art. When two or more nucleic acid molecules are to be co-expressed (e.g., both an antibody heavy and light chain or an antibody VH and VL), both nucleic acid molecules can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The introduction of nucleic acid molecules into a host cell can be confirmed using methods well known in the art. Such methods include, for example, nucleic acid analysis such as Northern blots or polymerase chain reaction (PCR) amplification of mRNA, immunoblotting for expression of gene products, or other suitable analytical methods to test the expression of an introduced nucleic acid sequence or its corresponding gene product. It is understood by those skilled in the art that the nucleic acid molecules are expressed in a sufficient amount to produce a desired product and it is further understood that expression levels can be optimized to obtain sufficient expression using methods well known in the art.

The term “host” as used herein refers to an animal, such as a mammal (e.g., a human).

The term “host cell” as used herein refers to a particular subject cell that may be transfected with a nucleic acid molecule and the progeny or potential progeny of such a cell. Progeny of such a cell may not be identical to the parent cell transfected with the nucleic acid molecule due to mutations or environmental influences that may occur in succeeding generations or integration of the nucleic acid molecule into the host cell genome.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The term “pharmaceutically acceptable” as used herein means being approved by a regulatory agency of the Federal or a state government, or listed in United States Pharmacopeia, European Pharmacopeia, or other generally recognized Pharmacopeia for use in animals, and more particularly in humans.

“Excipient” means a pharmaceutically-acceptable material, composition, or vehicle, such as a liquid or solid filler, diluent, solvent, or encapsulating material. Excipients include, for example, encapsulating materials or additives such as absorption accelerators, antioxidants, binders, buffers, carriers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents and mixtures thereof. The term “excipient” can also refer to a diluent, adjuvant (e.g., Freunds' adjuvant (complete or incomplete) or vehicle.

In some embodiments, excipients are pharmaceutically acceptable excipients. Examples of pharmaceutically acceptable excipients include buffers, such as phosphate, citrate, and other organic acids; antioxidants, including ascorbic acid; low molecular weight (e.g., fewer than about 10 amino acid residues) polypeptide; proteins, such as serum albumin, gelatin, or immunoglobulins; hydrophilic polymers, such as polyvinylpyrrolidone; amino acids, such as glycine, glutamine, asparagine, arginine, or lysine; monosaccharides, disaccharides, and other carbohydrates, including glucose, mannose, or dextrins; chelating agents, such as EDTA; sugar alcohols, such as mannitol or sorbitol; salt-forming counterions, such as sodium; and/or nonionic surfactants, such as TWEEN™, polyethylene glycol (PEG), and PLURONICS™. Other examples of pharmaceutically acceptable excipients are described in Remington and Gennaro, Remington's Pharmaceutical Sciences (18th ed. 1990).

In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Lippincott Williams & Wilkins: Philadelphia, PA, 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, FL, 2009. In some embodiments, pharmaceutically acceptable excipients are nontoxic to the cell or mammal being exposed thereto at the dosages and concentrations employed. In some embodiments, a pharmaceutically acceptable excipient is an aqueous pH buffered solution.

In some embodiments, excipients are sterile liquids, such as water and oils, including those of petroleum, animal, vegetable, or synthetic origin, such as peanut oil, soybean oil, mineral oil, sesame oil, and the like. Water is an exemplary excipient when a composition (e.g., a pharmaceutical composition) is administered intravenously. Saline solutions and aqueous dextrose and glycerol solutions can also be employed as liquid excipients, particularly for injectable solutions. An excipient can also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition, if desired, can also contain minor amounts of wetting or emulsifying agents, or pH buffering agents. Compositions can take the form of solutions, suspensions, emulsion, tablets, pills, capsules, powders, sustained-release formulations, and the like. Oral compositions, including formulations, can include standard excipients such as pharmaceutical grades of mannitol, lactose, starch, magnesium stearate, sodium saccharine, cellulose, magnesium carbonate, etc.

Compositions, including pharmaceutical compounds, may contain a prophylactically or therapeutically effective amount of a LILRB1 and/or LILRB2 binding agent (e.g., an antibody), for example, in isolated or purified form, together with a suitable amount of excipient so as to provide the form for proper administration to the subject (e.g., patient). The formulation should suit the mode of administration.

An “effective amount” is generally an amount sufficient to reduce the severity and/or frequency of symptoms, eliminate the symptoms and/or underlying cause, prevent or delay the occurrence of symptoms and/or their underlying cause, and/or improve or remediate the damage that results from or is associated with a disease, disorder, or condition. In some embodiments, the effective amount is a therapeutically effective amount or a prophylactically effective amount.

The term “therapeutically effective amount” as used herein refers to the amount of an agent (e.g., an antibody described herein or any other agent described herein) that is sufficient to reduce and/or ameliorate the severity and/or duration of a given disease, disorder or condition, and/or a symptom related thereto. A therapeutically effective amount of an agent, including a therapeutic agent, can be an amount necessary for (i) reduction, delay or amelioration of the advancement or progression of a given disease, disorder, or condition, (ii) reduction, delay or amelioration of the recurrence, development or onset of a given disease, disorder or conditions, and/or (iii) to improve or enhance the prophylactic or therapeutic effect of another therapy (e.g., a therapy other than the administration of an antibody described herein). A “therapeutically effective amount” of a substance/molecule/agent of the present disclosure (e.g., an anti-LILRB1 antibody, an anti-LILRB2 antibody, or a multispecific antibody that bind to both LILRB1 and LILRB2) may vary according to factors such as the disease state, age, sex, and weight of the individual, and the ability of the substance/molecule/agent, to elicit a desired response in the individual. A therapeutically effective amount encompasses an amount in which any toxic or detrimental effects of the substance/molecule/agent are outweighed by the therapeutically beneficial effects. In certain embodiments, the term “therapeutically effective amount” refers to an amount of a binding agent effective to “treat” a disease, disorder, or condition, in a subject or mammal.

The terms “subject” and “patient” may be used interchangeably. As used herein, in certain embodiments, a subject is a mammal, such as a non-primate or a primate (e.g., human). In specific embodiments, the subject is a human. In one embodiment, the subject is a mammal, e.g., a human, diagnosed with a disease or disorder. In another embodiment, the subject is a mammal, e.g., a human, at risk of developing a disease or disorder.

“Administer” or “administration” refers to the act of injecting or otherwise physically delivering a substance as it exists outside the body into a patient, such as by mucosal, intradermal, intravenous, intramuscular delivery, and/or any other method of physical delivery described herein or known in the art.

As used herein, the terms “treat,” “treatment” and “treating” refer to the reduction or amelioration of the progression, severity, and/or duration a given disease, disorder or condition, and/or a symptom related thereto, such as (i) reduction, delay or amelioration of the advancement or progression of a given disease, disorder, or condition, (ii) reduction, delay or amelioration of the recurrence, development or onset of a given disease, disorder or conditions, and/or (iii) to improve or enhance the prophylactic or therapeutic effect of another therapy (e.g., a therapy other than the administration of a binding agent described herein). The term “treating” includes both managing and ameliorating the disease. The terms “manage,” “managing,” and “management” refer to the beneficial effects that a subject derives from a therapy which does not necessarily result in a cure of the disease.

A “prophylactically effective amount” is an amount of a pharmaceutical composition that, when administered to a subject, will have the intended prophylactic effect, e.g., preventing or delaying the onset (or reoccurrence) of a disease, disorder or condition, or reducing the likelihood of the onset (or reoccurrence) of a disease, disorder, or condition or associated symptom(s).

The full therapeutic or prophylactic effect does not necessarily occur by administration of one dose, and may occur only after administration of a series of doses. Thus, a therapeutically or prophylactically effective amount may be administered in one or more administrations.

The terms “prevent,” “preventing,” and “prevention” refer to reducing the likelihood of the onset (or recurrence) of a disease, disorder, condition, or associated symptom(s) (e.g., diabetes or a cancer).

The term “immune response” as used herein includes responses from both the innate immune system and the adaptive immune system. It includes both cell-mediated and/or humoral immune responses. It includes both T-cell and B-cell responses, as well as responses from other cells of the immune system such as natural killer (NK) cells, monocytes, macrophages, dendritic cells, etc.

As used herein, “delaying” the development of cancer means to defer, hinder, slow, retard, stabilize, and/or postpone development of the disease. This delay can be of varying lengths of time, depending on the history of the disease and/or individual being treated. As is evident to one skilled in the art, a sufficient or significant delay can, in effect, encompass prevention, in that the individual does not develop the disease. A method that “delays” development of cancer is a method that reduces probability of disease development in a given time frame and/or reduces the extent of the disease in a given time frame, when compared to not using the method. Such comparisons are typically based on clinical studies, using a statistically significant number of individuals. Cancer development can be detectable using standard methods, including, but not limited to, computerized axial tomography (CAT Scan), Magnetic Resonance Imaging (MRI), abdominal ultrasound, clotting tests, arteriography, or biopsy. Development may also refer to cancer progression that may be initially undetectable and includes occurrence, recurrence, and onset.

The terms “about” and “approximately” mean within 20%, within 15%, within 10%, within 9%, within 8%, within 7%, within 6%, within 5%, within 4%, within 3%, within 2%, within 1%, or less of a given value or range.

As used herein, comparative terms as used herein, such as reduce, decrease, increase, or any grammatical variation thereof, can refer to certain variation from the reference. In some embodiments, such variation can refer to about 10%, or about 20%, or about 30%, or about 40%, or about 50%, or about 60%, or about 70%, or about 80%, or about 90%, or about 1 fold, or about 2 fold, or about 3 fold, or about 4 fold, or about 5 fold, or about 10 fold, or about 20 fold, or about 30 fold, or about 40 fold, or about 100 fold or higher than the reference. In some embodiments, such variation can refer to about 1%, or about 2%, or about 3%, or about 4%, or about 5%, or about 6%, or about 7%, or about 8%, or about 9%, or about 10%, or about 20%, or about 30%, or about 40%, or about 50%, or about 60%, or about 70%, or about 80%, or about 90%, or about 95%, or about 96%, or about 97%, or about 98%, or about 99% of the reference.

As used in the present disclosure and claims, the singular forms “a”, “an” and “the” include plural forms unless the context clearly dictates otherwise.

In some embodiments, the terms “first,” “second,” “third,” “fourth” and similar in a component name are used to distinguish and identify more than one component sharing certain identity in their names. For example, “first antibody” and “second antibody” are used to distinguish two antibodies.

It is understood that wherever embodiments are described herein with the term “comprising” otherwise analogous embodiments described in terms of “consisting of” and/or “consisting essentially of” are also provided. It is also understood that wherever embodiments are described herein with the phrase “consisting essentially of” otherwise analogous embodiments described in terms of “consisting of” are also provided.

The term “between” as used in a phrase as such “between A and B” or “between A-B” refers to a range including both A and B.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

The term “optional” or “optionally” means that the subsequently described circumstance may or may not occur, so that the description includes instances wherein the circumstance occurs, and the instances wherein the circumstance does not occur.

### 5.2. Binding Molecules

5.2.1. Antibodies that Bind to LILRB1

In one aspect, provided herein are binding agents (e.g., antibodies including fragments thereof) capable of binding to LILRB1. In some embodiments, the antibodies provided herein bind to human LILRB1. In some embodiments, the antibodies provided herein bind to rhesus macaque LILRB1. In some embodiments, the antibodies provided herein bind to cynomolgus LILRB1.

As used herein, LILRB1 refers to a LILRB1 polypeptide, a LILRB1 polypeptide fragment, a LILRB1 peptide or a LILRB1 epitope. In some embodiments, the LILRB1 binding agents are derived from human or humanized antibodies (e.g., comprising human framework regions) that bind LILRB1, including a LILRB1 polypeptide, a LILRB1 polypeptide fragment, a LILRB1 peptide or a LILRB1 epitope. In some embodiments, the binding agent (e.g., an antibody including fragment thereof) can bind to LILRB1 expressed on the surface of a mammalian (e.g., human) cell, including a LILRB1 expressing immune cell (e.g., an NK cell or a T cell). In some embodiments, the binding agent (e.g., an antibody including fragment thereof) provided herein binds a LILRB1 extracellular epitope exposed on a cell such as an immune cell. In some embodiments, described herein is a binding agent (e.g., an antibody including fragment thereof) that binds to LILRB1, such as human LILRB1 or a portion thereof. In some embodiments, LILRB1 is a human LILRB1. In some embodiments, the binding agent provided herein is a human LILRB1 binding agent (e.g., an antibody that binds to human LILRB1).

In some embodiments, the anti-LILRB1 antibody provided herein is an antagonist antibody. In one embodiment, the antibodies according to the disclosure are LILRB1 antagonists with no or low agonistic activity. In another embodiment, the antibody or functional fragment comprising an antigen-binding portion binds the target protein LILRB1 and decreases the binding of ligands of LILRB1 to LILRB1 to a basal level. In one aspect of this embodiment, the antibody or functional fragment reduces the amount of ligands that bind to LILRB1. In a further aspect of this embodiment, the antibody or functional fragment completely prevents ligands of LILRB1 from binding to LILRB1. In some embodiments, the ligands are MHC I molecules. In some embodiments, the ligand is HLA-A2. In some embodiments, the ligand is HLA-G. In some embodiments, the anti-LILRB1 antibody or functional fragment provided herein inhibits one or more of these LILRB1 functional properties. An antibody that inhibits one or more of these LILRB1 functional properties (e.g. biochemical, immunochemical, cellular, physiological or other biological activities, or the like) as determined according to methodologies known to the art and described herein, will be understood to relate to a statistically significant decrease in the particular activity relative to that seen in the absence of the antibody (e.g. or when a control antibody of irrelevant specificity is present). In some embodiments, an antibody that inhibits LILRB1 activity effects such a statistically significant decrease by at least 10% of the measured parameter, by at least 50%, 80% or 90%, and in certain embodiments an antibody of the disclosure may inhibit greater than 95%, 98% or 99% of LILRB1 functional activity.

In some embodiments, the anti-LILRB1 antibody provided herein binds to LILRB1 (e.g., human LILRB1) with a dissociation constant (K_{D}) of ≤1 μM, ≤100 nM, ≤10 nM, ≤1 nM, ≤0.1 nM, ≤0.01 nM, or ≤0.001 nM (e.g. 10⁻⁸ M or less, e.g. from 10⁻⁸ M to 10⁻¹³ M, e.g., from 10⁻⁹ M to 10⁻¹³ M). A variety of methods of measuring binding affinity are known in the art, any of which can be used for purposes of the present disclosure, including by RIA, for example, performed with the Fab version of an antibody of interest and its antigen (Chen et al., 1999, J. Mol Biol 293:865-81); by biolayer interferometry (BLI) or surface plasmon resonance (SPR) assays by Octet®, using, for example, an Octet®Red96 system, or by Biacore®, using, for example, a Biacore®TM-2000 or a Biacore®TM-3000. An “on-rate” or “rate of association” or “association rate” or “kₒₙ” may also be determined with the same biolayer interferometry (BLI) or surface plasmon resonance (SPR) techniques described above using, for example, the OCTET®Red96, the BIACORE®TM-2000, the BIACORE®TM-3000 system, the BIACORE®TM-8K, or the BIACORE®TM-8K+ system.

In one embodiment, the anti-LILRB1 antibodies of the disclosure do not cross-react with one or more LILRB or LILRA family members (e.g. LILRB2, LILRA1, LILRA2, etc.). In one embodiment, the anti-LILRB1 antibodies of the disclosure cross-react with one or more LILRB and LILRA family members (e.g. LILRB2, LILRA1, LILRA2, etc.).

In some embodiments, the anti-LILRB1 antibodies provide herein are those described in Section 7 below. Thus, in some embodiments, the antibody provided herein comprises one or more CDR sequences of any one of SEQ ID NOs: 283-400, and 1155-1166. CDR sequences can be determined according to well-known numbering systems. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the anti-LILRB1 antibody is humanized. In some embodiments, the anti-LILRB1 antibody comprises an acceptor human framework, e.g., a human immunoglobulin framework or a human consensus framework.

In some embodiments, the anti-LILRB1 antibody provided herein comprises an a HCDR1, a HCDR2, and a HCDR3 as set forth in a VH comprising the amino acid sequence of SEQ ID NO: 283. In some embodiments, the anti-LILRB1 antibody provided herein comprises a HCDR1, a HCDR2, and a HCDR3 as set forth in a VH comprising the amino acid sequence of SEQ ID NO: 285. In some embodiments, the anti-LILRB1 antibody provided herein comprises a HCDR1, a HCDR2, and a HCDR3 as set forth in a VH comprising the amino acid sequence of SEQ ID NO: 287. In some embodiments, the anti-LILRB1 antibody provided herein comprises a HCDR1, a HCDR2, and a HCDR3 as set forth in a VH comprising the amino acid sequence of SEQ ID NO: 289. In some embodiments, the anti-LILRB1 antibody provided herein comprises a HCDR1, a HCDR2, and a HCDR3 as set forth in a VH comprising the amino acid sequence of SEQ ID NO: 291. In some embodiments, the anti-LILRB1 antibody provided herein comprises a HCDR1, a HCDR2, and a HCDR3 as set forth in a VH comprising the amino acid sequence of SEQ ID NO: 293. In some embodiments, the anti-LILRB1 antibody provided herein comprises a HCDR1, a HCDR2, and a HCDR3 as set forth in a VH comprising the amino acid sequence of SEQ ID NO: 295. In some embodiments, the anti-LILRB1 antibody provided herein comprises a HCDR1, a HCDR2, and a HCDR3 as set forth in a VH comprising the amino acid sequence of SEQ ID NO: 297. In some embodiments, the anti-LILRB1 antibody provided herein comprises a HCDR1, a HCDR2, and a HCDR3 as set forth in a VH comprising the amino acid sequence of SEQ ID NO: 299. In some embodiments, the anti-LILRB1 antibody provided herein comprises a HCDR1, a HCDR2, and a HCDR3 as set forth in a VH comprising the amino acid sequence of SEQ ID NO: 301. In some embodiments, the anti-LILRB1 antibody provided herein comprises a HCDR1, a HCDR2, and a HCDR3 as set forth in a VH comprising the amino acid sequence of SEQ ID NO: 303. In some embodiments, the anti-LILRB1 antibody provided herein comprises a HCDR1, a HCDR2, and a HCDR3 as set forth in a VH comprising the amino acid sequence of SEQ ID NO: 305. In some embodiments, the anti-LILRB1 antibody provided herein comprises a HCDR1, a HCDR2, and a HCDR3 as set forth in a VH comprising the amino acid sequence of SEQ ID NO: 307. In some embodiments, the anti-LILRB1 antibody provided herein comprises a HCDR1, a HCDR2, and a HCDR3 as set forth in a VH comprising the amino acid sequence of SEQ ID NO: 309. In some embodiments, the anti-LILRB1 antibody provided herein comprises a HCDR1, a HCDR2, and a HCDR3 as set forth in a VH comprising the amino acid sequence of SEQ ID NO: 311. In some embodiments, the anti-LILRB1 antibody provided herein comprises a HCDR1, a HCDR2, and a HCDR3 as set forth in a VH comprising the amino acid sequence of SEQ ID NO: 313. In some embodiments, the anti-LILRB1 antibody provided herein comprises a HCDR1, a HCDR2, and a HCDR3 as set forth in a VH comprising the amino acid sequence of SEQ ID NO: 315. In some embodiments, the anti-LILRB1 antibody provided herein comprises a HCDR1, a HCDR2, and a HCDR3 as set forth in a VH comprising the amino acid sequence of SEQ ID NO: 317. In some embodiments, the anti-LILRB1 antibody provided herein comprises a HCDR1, a HCDR2, and a HCDR3 as set forth in a VH comprising the amino acid sequence of SEQ ID NO: 319. In some embodiments, the anti-LILRB1 antibody provided herein comprises a HCDR1, a HCDR2, and a HCDR3 as set forth in a VH comprising the amino acid sequence of SEQ ID NO: 321. In some embodiments, the anti-LILRB1 antibody provided herein comprises a HCDR1, a HCDR2, and a HCDR3 as set forth in a VH comprising the amino acid sequence of SEQ ID NO: 323. In some embodiments, the anti-LILRB1 antibody provided herein comprises a HCDR1, a HCDR2, and a HCDR3 as set forth in a VH comprising the amino acid sequence of SEQ ID NO: 325. In some embodiments, the anti-LILRB1 antibody provided herein comprises a HCDR1, a HCDR2, and a HCDR3 as set forth in a VH comprising the amino acid sequence of SEQ ID NO: 327. In some embodiments, the anti-LILRB1 antibody provided herein comprises a HCDR1, a HCDR2, and a HCDR3 as set forth in a VH comprising the amino acid sequence of SEQ ID NO: 329. In some embodiments, the anti-LILRB1 antibody provided herein comprises a HCDR1, a HCDR2, and a HCDR3 as set forth in a VH comprising the amino acid sequence of SEQ ID NO: 331. In some embodiments, the anti-LILRB1 antibody provided herein comprises a HCDR1, a HCDR2, and a HCDR3 as set forth in a VH comprising the amino acid sequence of SEQ ID NO: 333. In some embodiments, the anti-LILRB1 antibody provided herein comprises a HCDR1, a HCDR2, and a HCDR3 as set forth in a VH comprising the amino acid sequence of SEQ ID NO: 335. In some embodiments, the anti-LILRB1 antibody provided herein comprises a HCDR1, a HCDR2, and a HCDR3 as set forth in a VH comprising the amino acid sequence of SEQ ID NO: 337. In some embodiments, the anti-LILRB1 antibody provided herein comprises a HCDR1, a HCDR2, and a HCDR3 as set forth in a VH comprising the amino acid sequence of SEQ ID NO: 339. In some embodiments, the anti-LILRB1 antibody provided herein comprises a HCDR1, a HCDR2, and a HCDR3 as set forth in a VH comprising the amino acid sequence of SEQ ID NO: 341. In some embodiments, the anti-LILRB1 antibody provided herein comprises a HCDR1, a HCDR2, and a HCDR3 as set forth in a VH comprising the amino acid sequence of SEQ ID NO: 343. In some embodiments, the anti-LILRB1 antibody provided herein comprises a HCDR1, a HCDR2, and a HCDR3 as set forth in a VH comprising the amino acid sequence of SEQ ID NO: 345. In some embodiments, the anti-LILRB1 antibody provided herein comprises a HCDR1, a HCDR2, and a HCDR3 as set forth in a VH comprising the amino acid sequence of SEQ ID NO: 347. In some embodiments, the anti-LILRB1 antibody provided herein comprises a HCDR1, a HCDR2, and a HCDR3 as set forth in a VH comprising the amino acid sequence of SEQ ID NO: 349. In some embodiments, the anti-LILRB1 antibody provided herein comprises a HCDR1, a HCDR2, and a HCDR3 as set forth in a VH comprising the amino acid sequence of SEQ ID NO: 351. In some embodiments, the anti-LILRB1 antibody provided herein comprises a HCDR1, a HCDR2, and a HCDR3 as set forth in a VH comprising the amino acid sequence of SEQ ID NO: 353. In some embodiments, the anti-LILRB1 antibody provided herein comprises a HCDR1, a HCDR2, and a HCDR3 as set forth in a VH comprising the amino acid sequence of SEQ ID NO: 355. In some embodiments, the anti-LILRB1 antibody provided herein comprises a HCDR1, a HCDR2, and a HCDR3 as set forth in a VH comprising the amino acid sequence of SEQ ID NO: 357. In some embodiments, the anti-LILRB1 antibody provided herein comprises a HCDR1, a HCDR2, and a HCDR3 as set forth in a VH comprising the amino acid sequence of SEQ ID NO: 359. In some embodiments, the anti-LILRB1 antibody provided herein comprises a HCDR1, a HCDR2, and a HCDR3 as set forth in a VH comprising the amino acid sequence of SEQ ID NO: 361. In some embodiments, the anti-LILRB1 antibody provided herein comprises a HCDR1, a HCDR2, and a HCDR3 as set forth in a VH comprising the amino acid sequence of SEQ ID NO: 363. In some embodiments, the anti-LILRB1 antibody provided herein comprises a HCDR1, a HCDR2, and a HCDR3 as set forth in a VH comprising the amino acid sequence of SEQ ID NO: 365. In some embodiments, the anti-LILRB1 antibody provided herein comprises a HCDR1, a HCDR2, and a HCDR3 as set forth in a VH comprising the amino acid sequence of SEQ ID NO: 367. In some embodiments, the anti-LILRB1 antibody provided herein comprises a HCDR1, a HCDR2, and a HCDR3 as set forth in a VH comprising the amino acid sequence of SEQ ID NO: 369. In some embodiments, the anti-LILRB1 antibody provided herein comprises a HCDR1, a HCDR2, and a HCDR3 as set forth in a VH comprising the amino acid sequence of SEQ ID NO: 371. In some embodiments, the anti-LILRB1 antibody provided herein comprises a HCDR1, a HCDR2, and a HCDR3 as set forth in a VH comprising the amino acid sequence of SEQ ID NO: 373. In some embodiments, the anti-LILRB1 antibody provided herein comprises a HCDR1, a HCDR2, and a HCDR3 as set forth in a VH comprising the amino acid sequence of SEQ ID NO: 375. In some embodiments, the anti-LILRB1 antibody provided herein comprises a HCDR1, a HCDR2, and a HCDR3 as set forth in a VH comprising the amino acid sequence of SEQ ID NO: 377. In some embodiments, the anti-LILRB1 antibody provided herein comprises a HCDR1, a HCDR2, and a HCDR3 as set forth in a VH comprising the amino acid sequence of SEQ ID NO: 379. In some embodiments, the anti-LILRB1 antibody provided herein comprises a HCDR1, a HCDR2, and a HCDR3 as set forth in a VH comprising the amino acid sequence of SEQ ID NO: 381. In some embodiments, the anti-LILRB1 antibody provided herein comprises a HCDR1, a HCDR2, and a HCDR3 as set forth in a VH comprising the amino acid sequence of SEQ ID NO: 383. In some embodiments, the anti-LILRB1 antibody provided herein comprises a HCDR1, a HCDR2, and a HCDR3 as set forth in a VH comprising the amino acid sequence of SEQ ID NO: 385. In some embodiments, the anti-LILRB1 antibody provided herein comprises a HCDR1, a HCDR2, and a HCDR3 as set forth in a VH comprising the amino acid sequence of SEQ ID NO: 387. In some embodiments, the anti-LILRB1 antibody provided herein comprises a HCDR1, a HCDR2, and a HCDR3 as set forth in a VH comprising the amino acid sequence of SEQ ID NO: 389. In some embodiments, the anti-LILRB1 antibody provided herein comprises a HCDR1, a HCDR2, and a HCDR3 as set forth in a VH comprising the amino acid sequence of SEQ ID NO: 391. In some embodiments, the anti-LILRB1 antibody provided herein comprises a HCDR1, a HCDR2, and a HCDR3 as set forth in a VH comprising the amino acid sequence of SEQ ID NO: 393. In some embodiments, the anti-LILRB1 antibody provided herein comprises a HCDR1, a HCDR2, and a HCDR3 as set forth in a VH comprising the amino acid sequence of SEQ ID NO: 395. In some embodiments, the anti-LILRB1 antibody provided herein comprises a HCDR1, a HCDR2, and a HCDR3 as set forth in a VH comprising the amino acid sequence of SEQ ID NO: 397. In some embodiments, the anti-LILRB1 antibody provided herein comprises a HCDR1, a HCDR2, and a HCDR3 as set forth in a VH comprising the amino acid sequence of SEQ ID NO: 399. In some embodiments, the anti-LILRB1 antibody provided herein comprises a HCDR1, a HCDR2, and a HCDR3 as set forth in a VH comprising the amino acid sequence of SEQ ID NO: 1155. In some embodiments, the anti-LILRB1 antibody provided herein comprises a HCDR1, a HCDR2, and a HCDR3 as set forth in a VH comprising the amino acid sequence of SEQ ID NO: 1157. In some embodiments, the anti-LILRB1 antibody provided herein comprises a HCDR1, a HCDR2, and a HCDR3 as set forth in a VH comprising the amino acid sequence of SEQ ID NO: 1159. In some embodiments, the anti-LILRB1 antibody provided herein comprises a HCDR1, a HCDR2, and a HCDR3 as set forth in a VH comprising the amino acid sequence of SEQ ID NO: 1161. In some embodiments, the anti-LILRB1 antibody provided herein comprises a HCDR1, a HCDR2, and a HCDR3 as set forth in a VH comprising the amino acid sequence of SEQ ID NO: 1163. In some embodiments, the anti-LILRB1 antibody provided herein comprises a HCDR1, a HCDR2, and a HCDR3 as set forth in a VH comprising the amino acid sequence of SEQ ID NO: 1165. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering.

In some embodiments, the anti-LILRB1 antibody provided herein comprises a LCDR1, a LCDR2, and a LCDR3 as set forth in a VL comprising the amino acid sequence of SEQ ID NO: 284. In some embodiments, the anti-LILRB1 antibody provided herein comprises a LCDR1, a LCDR2, and a LCDR3 as set forth in a VL comprising the amino acid sequence of SEQ ID NO: 286. In some embodiments, the anti-LILRB1 antibody provided herein comprises a LCDR1, a LCDR2, and a LCDR3 as set forth in a VL comprising the amino acid sequence of SEQ ID NO: 288. In some embodiments, the anti-LILRB1 antibody provided herein comprises a LCDR1, a LCDR2, and a LCDR3 as set forth in a VL comprising the amino acid sequence of SEQ ID NO: 290. In some embodiments, the anti-LILRB1 antibody provided herein comprises a LCDR1, a LCDR2, and a LCDR3 as set forth in a VL comprising the amino acid sequence of SEQ ID NO: 292. In some embodiments, the anti-LILRB1 antibody provided herein comprises a LCDR1, a LCDR2, and a LCDR3 as set forth in a VL comprising the amino acid sequence of SEQ ID NO: 294. In some embodiments, the anti-LILRB1 antibody provided herein comprises a LCDR1, a LCDR2, and a LCDR3 as set forth in a VL comprising the amino acid sequence of SEQ ID NO: 296. In some embodiments, the anti-LILRB1 antibody provided herein comprises a LCDR1, a LCDR2, and a LCDR3 as set forth in a VL comprising the amino acid sequence of SEQ ID NO: 298. In some embodiments, the anti-LILRB1 antibody provided herein comprises a LCDR1, a LCDR2, and a LCDR3 as set forth in a VL comprising the amino acid sequence of SEQ ID NO: 300. In some embodiments, the anti-LILRB1 antibody provided herein comprises a LCDR1, a LCDR2, and a LCDR3 as set forth in a VL comprising the amino acid sequence of SEQ ID NO: 302. In some embodiments, the anti-LILRB1 antibody provided herein comprises a LCDR1, a LCDR2, and a LCDR3 as set forth in a VL comprising the amino acid sequence of SEQ ID NO: 304. In some embodiments, the anti-LILRB1 antibody provided herein comprises a LCDR1, a LCDR2, and a LCDR3 as set forth in a VL comprising the amino acid sequence of SEQ ID NO: 306. In some embodiments, the anti-LILRB1 antibody provided herein comprises a LCDR1, a LCDR2, and a LCDR3 as set forth in a VL comprising the amino acid sequence of SEQ ID NO: 308. In some embodiments, the anti-LILRB1 antibody provided herein comprises a LCDR1, a LCDR2, and a LCDR3 as set forth in a VL comprising the amino acid sequence of SEQ ID NO: 310. In some embodiments, the anti-LILRB1 antibody provided herein comprises a LCDR1, a LCDR2, and a LCDR3 as set forth in a VL comprising the amino acid sequence of SEQ ID NO: 312. In some embodiments, the anti-LILRB1 antibody provided herein comprises a LCDR1, a LCDR2, and a LCDR3 as set forth in a VL comprising the amino acid sequence of SEQ ID NO: 314. In some embodiments, the anti-LILRB1 antibody provided herein comprises a LCDR1, a LCDR2, and a LCDR3 as set forth in a VL comprising the amino acid sequence of SEQ ID NO: 316. In some embodiments, the anti-LILRB1 antibody provided herein comprises a LCDR1, a LCDR2, and a LCDR3 as set forth in a VL comprising the amino acid sequence of SEQ ID NO: 318. In some embodiments, the anti-LILRB1 antibody provided herein comprises a LCDR1, a LCDR2, and a LCDR3 as set forth in a VL comprising the amino acid sequence of SEQ ID NO: 320. In some embodiments, the anti-LILRB1 antibody provided herein comprises a LCDR1, a LCDR2, and a LCDR3 as set forth in a VL comprising the amino acid sequence of SEQ ID NO: 322. In some embodiments, the anti-LILRB1 antibody provided herein comprises a LCDR1, a LCDR2, and a LCDR3 as set forth in a VL comprising the amino acid sequence of SEQ ID NO: 324. In some embodiments, the anti-LILRB1 antibody provided herein comprises a LCDR1, a LCDR2, and a LCDR3 as set forth in a VL comprising the amino acid sequence of SEQ ID NO: 326. In some embodiments, the anti-LILRB1 antibody provided herein comprises a LCDR1, a LCDR2, and a LCDR3 as set forth in a VL comprising the amino acid sequence of SEQ ID NO: 328. In some embodiments, the anti-LILRB1 antibody provided herein comprises a LCDR1, a LCDR2, and a LCDR3 as set forth in a VL comprising the amino acid sequence of SEQ ID NO: 330. In some embodiments, the anti-LILRB1 antibody provided herein comprises a LCDR1, a LCDR2, and a LCDR3 as set forth in a VL comprising the amino acid sequence of SEQ ID NO: 332. In some embodiments, the anti-LILRB1 antibody provided herein comprises a LCDR1, a LCDR2, and a LCDR3 as set forth in a VL comprising the amino acid sequence of SEQ ID NO: 334. In some embodiments, the anti-LILRB1 antibody provided herein comprises a LCDR1, a LCDR2, and a LCDR3 as set forth in a VL comprising the amino acid sequence of SEQ ID NO: 336. In some embodiments, the anti-LILRB1 antibody provided herein comprises a LCDR1, a LCDR2, and a LCDR3 as set forth in a VL comprising the amino acid sequence of SEQ ID NO: 338. In some embodiments, the anti-LILRB1 antibody provided herein comprises a LCDR1, a LCDR2, and a LCDR3 as set forth in a VL comprising the amino acid sequence of SEQ ID NO: 340. In some embodiments, the anti-LILRB1 antibody provided herein comprises a LCDR1, a LCDR2, and a LCDR3 as set forth in a VL comprising the amino acid sequence of SEQ ID NO: 342. In some embodiments, the anti-LILRB1 antibody provided herein comprises a LCDR1, a LCDR2, and a LCDR3 as set forth in a VL comprising the amino acid sequence of SEQ ID NO: 344. In some embodiments, the anti-LILRB1 antibody provided herein comprises a LCDR1, a LCDR2, and a LCDR3 as set forth in a VL comprising the amino acid sequence of SEQ ID NO: 346. In some embodiments, the anti-LILRB1 antibody provided herein comprises a LCDR1, a LCDR2, and a LCDR3 as set forth in a VL comprising the amino acid sequence of SEQ ID NO: 348. In some embodiments, the anti-LILRB1 antibody provided herein comprises a LCDR1, a LCDR2, and a LCDR3 as set forth in a VL comprising the amino acid sequence of SEQ ID NO: 350. In some embodiments, the anti-LILRB1 antibody provided herein comprises a LCDR1, a LCDR2, and a LCDR3 as set forth in a VL comprising the amino acid sequence of SEQ ID NO: 352. In some embodiments, the anti-LILRB1 antibody provided herein comprises a LCDR1, a LCDR2, and a LCDR3 as set forth in a VL comprising the amino acid sequence of SEQ ID NO: 354. In some embodiments, the anti-LILRB1 antibody provided herein comprises a LCDR1, a LCDR2, and a LCDR3 as set forth in a VL comprising the amino acid sequence of SEQ ID NO: 356. In some embodiments, the anti-LILRB1 antibody provided herein comprises a LCDR1, a LCDR2, and a LCDR3 as set forth in a VL comprising the amino acid sequence of SEQ ID NO: 358. In some embodiments, the anti-LILRB1 antibody provided herein comprises a LCDR1, a LCDR2, and a LCDR3 as set forth in a VL comprising the amino acid sequence of SEQ ID NO: 360. In some embodiments, the anti-LILRB1 antibody provided herein comprises a LCDR1, a LCDR2, and a LCDR3 as set forth in a VL comprising the amino acid sequence of SEQ ID NO: 362. In some embodiments, the anti-LILRB1 antibody provided herein comprises a LCDR1, a LCDR2, and a LCDR3 as set forth in a VL comprising the amino acid sequence of SEQ ID NO: 364. In some embodiments, the anti-LILRB1 antibody provided herein comprises a LCDR1, a LCDR2, and a LCDR3 as set forth in a VL comprising the amino acid sequence of SEQ ID NO: 366. In some embodiments, the anti-LILRB1 antibody provided herein comprises a LCDR1, a LCDR2, and a LCDR3 as set forth in a VL comprising the amino acid sequence of SEQ ID NO: 368. In some embodiments, the anti-LILRB1 antibody provided herein comprises a LCDR1, a LCDR2, and a LCDR3 as set forth in a VL comprising the amino acid sequence of SEQ ID NO: 370. In some embodiments, the anti-LILRB1 antibody provided herein comprises a LCDR1, a LCDR2, and a LCDR3 as set forth in a VL comprising the amino acid sequence of SEQ ID NO: 372. In some embodiments, the anti-LILRB1 antibody provided herein comprises a LCDR1, a LCDR2, and a LCDR3 as set forth in a VL comprising the amino acid sequence of SEQ ID NO: 374. In some embodiments, the anti-LILRB1 antibody provided herein comprises a LCDR1, a LCDR2, and a LCDR3 as set forth in a VL comprising the amino acid sequence of SEQ ID NO: 376. In some embodiments, the anti-LILRB1 antibody provided herein comprises a LCDR1, a LCDR2, and a LCDR3 as set forth in a VL comprising the amino acid sequence of SEQ ID NO: 378. In some embodiments, the anti-LILRB1 antibody provided herein comprises a LCDR1, a LCDR2, and a LCDR3 as set forth in a VL comprising the amino acid sequence of SEQ ID NO: 380. In some embodiments, the anti-LILRB1 antibody provided herein comprises a LCDR1, a LCDR2, and a LCDR3 as set forth in a VL comprising the amino acid sequence of SEQ ID NO: 382. In some embodiments, the anti-LILRB1 antibody provided herein comprises a LCDR1, a LCDR2, and a LCDR3 as set forth in a VL comprising the amino acid sequence of SEQ ID NO: 384. In some embodiments, the anti-LILRB1 antibody provided herein comprises a LCDR1, a LCDR2, and a LCDR3 as set forth in a VL comprising the amino acid sequence of SEQ ID NO: 386. In some embodiments, the anti-LILRB1 antibody provided herein comprises a LCDR1, a LCDR2, and a LCDR3 as set forth in a VL comprising the amino acid sequence of SEQ ID NO: 388. In some embodiments, the anti-LILRB1 antibody provided herein comprises a LCDR1, a LCDR2, and a LCDR3 as set forth in a VL comprising the amino acid sequence of SEQ ID NO: 390. In some embodiments, the anti-LILRB1 antibody provided herein comprises a LCDR1, a LCDR2, and a LCDR3 as set forth in a VL comprising the amino acid sequence of SEQ ID NO: 392. In some embodiments, the anti-LILRB1 antibody provided herein comprises a LCDR1, a LCDR2, and a LCDR3 as set forth in a VL comprising the amino acid sequence of SEQ ID NO: 394. In some embodiments, the anti-LILRB1 antibody provided herein comprises a LCDR1, a LCDR2, and a LCDR3 as set forth in a VL comprising the amino acid sequence of SEQ ID NO: 396. In some embodiments, the anti-LILRB1 antibody provided herein comprises a LCDR1, a LCDR2, and a LCDR3 as set forth in a VL comprising the amino acid sequence of SEQ ID NO: 398. In some embodiments, the anti-LILRB1 antibody provided herein comprises a LCDR1, a LCDR2, and a LCDR3 as set forth in a VL comprising the amino acid sequence of SEQ ID NO: 400. In some embodiments, the anti-LILRB1 antibody provided herein comprises a LCDR1, a LCDR2, and a LCDR3 as set forth in a VL comprising the amino acid sequence of SEQ ID NO: 1156. In some embodiments, the anti-LILRB1 antibody provided herein comprises a LCDR1, a LCDR2, and a LCDR3 as set forth in a VL comprising the amino acid sequence of SEQ ID NO: 1158. In some embodiments, the anti-LILRB1 antibody provided herein comprises a LCDR1, a LCDR2, and a LCDR3 as set forth in a VL comprising the amino acid sequence of SEQ ID NO: 1160. In some embodiments, the anti-LILRB1 antibody provided herein comprises a LCDR1, a LCDR2, and a LCDR3 as set forth in a VL comprising the amino acid sequence of SEQ ID NO: 1162. In some embodiments, the anti-LILRB1 antibody provided herein comprises a LCDR1, a LCDR2, and a LCDR3 as set forth in a VL comprising the amino acid sequence of SEQ ID NO: 1164. In some embodiments, the anti-LILRB1 antibody provided herein comprises a LCDR1, a LCDR2, and a LCDR3 as set forth in a VL comprising the amino acid sequence of SEQ ID NO: 1166. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering.

In some embodiments, the antibody or antigen binding fragment provided herein comprises a HCDR1, a HCDR2, and/or a HCDR3 as set forth in a VH comprising the amino acid sequence of SEQ ID NO: 283, and/or a LCDR1, a LCDR2, and/or a LCDR3 as set forth in a VL comprising the amino acid sequence of SEQ ID NO: 284. In some embodiments, the antibody or antigen binding fragment provided herein comprises a HCDR1, a HCDR2, and a HCDR3 as set forth in a VH comprising the amino acid sequence of SEQ ID NO: 283, and a LCDR1, a LCDR2, and a LCDR3 as set forth in a VL comprising the amino acid sequence of SEQ ID NO: 284.

In some embodiments, the antibody or antigen binding fragment provided herein comprises a HCDR1, a HCDR2, and/or a HCDR3 as set forth in a VH comprising the amino acid sequence of SEQ ID NO: 285, and/or a LCDR1, a LCDR2, and/or a LCDR3 as set forth in a VL comprising the amino acid sequence of SEQ ID NO: 286. In some embodiments, the antibody or antigen binding fragment provided herein comprises a HCDR1, a HCDR2, and a HCDR3 as set forth in a VH comprising the amino acid sequence of SEQ ID NO: 285, and a LCDR1, a LCDR2, and a LCDR3 as set forth in a VL comprising the amino acid sequence of SEQ ID NO: 286.

In some embodiments, the antibody or antigen binding fragment provided herein comprises a HCDR1, a HCDR2, and/or a HCDR3 as set forth in a VH comprising the amino acid sequence of SEQ ID NO: 287, and/or a LCDR1, a LCDR2, and/or a LCDR3 as set forth in a VL comprising the amino acid sequence of SEQ ID NO: 288. In some embodiments, the antibody or antigen binding fragment provided herein comprises a HCDR1, a HCDR2, and a HCDR3 as set forth in a VH comprising the amino acid sequence of SEQ ID NO: 287, and a LCDR1, a LCDR2, and a LCDR3 as set forth in a VL comprising the amino acid sequence of SEQ ID NO: 288.

In some embodiments, the antibody or antigen binding fragment provided herein comprises a HCDR1, a HCDR2, and/or a HCDR3 as set forth in a VH comprising the amino acid sequence of SEQ ID NO: 289, and/or a LCDR1, a LCDR2, and/or a LCDR3 as set forth in a VL comprising the amino acid sequence of SEQ ID NO: 290. In some embodiments, the antibody or antigen binding fragment provided herein comprises a HCDR1, a HCDR2, and a HCDR3 as set forth in a VH comprising the amino acid sequence of SEQ ID NO: 289, and a LCDR1, a LCDR2, and a LCDR3 as set forth in a VL comprising the amino acid sequence of SEQ ID NO: 290.

In some embodiments, the antibody or antigen binding fragment provided herein comprises a HCDR1, a HCDR2, and/or a HCDR3 as set forth in a VH comprising the amino acid sequence of SEQ ID NO: 291, and/or a LCDR1, a LCDR2, and/or a LCDR3 as set forth in a VL comprising the amino acid sequence of SEQ ID NO: 292. In some embodiments, the antibody or antigen binding fragment provided herein comprises a HCDR1, a HCDR2, and a HCDR3 as set forth in a VH comprising the amino acid sequence of SEQ ID NO: 291, and a LCDR1, a LCDR2, and a LCDR3 as set forth in a VL comprising the amino acid sequence of SEQ ID NO: 292.

In some embodiments, the antibody or antigen binding fragment provided herein comprises a HCDR1, a HCDR2, and/or a HCDR3 as set forth in a VH comprising the amino acid sequence of SEQ ID NO: 293, and/or a LCDR1, a LCDR2, and/or a LCDR3 as set forth in a VL comprising the amino acid sequence of SEQ ID NO: 294. In some embodiments, the antibody or antigen binding fragment provided herein comprises a HCDR1, a HCDR2, and a HCDR3 as set forth in a VH comprising the amino acid sequence of SEQ ID NO: 293, and a LCDR1, a LCDR2, and a LCDR3 as set forth in a VL comprising the amino acid sequence of SEQ ID NO: 294.

In some embodiments, the antibody or antigen binding fragment provided herein sequence of SEQ ID NO: 295, and/or a LCDR1, a LCDR2, and/or a LCDR3 as set forth in a VL comprising the amino acid sequence of SEQ ID NO: 296. In some embodiments, the antibody or antigen binding fragment provided herein comprises a HCDR1, a HCDR2, and a HCDR3 as set forth in a VH comprising the amino acid sequence of SEQ ID NO: 295, and a LCDR1, a LCDR2, and a LCDR3 as set forth in a VL comprising the amino acid sequence of SEQ ID NO: 296.

In some embodiments, the antibody or antigen binding fragment provided herein comprises a HCDR1, a HCDR2, and/or a HCDR3 as set forth in a VH comprising the amino acid sequence of SEQ ID NO: 297, and/or a LCDR1, a LCDR2, and/or a LCDR3 as set forth in a VL comprising the amino acid sequence of SEQ ID NO: 298. In some embodiments, the antibody or antigen binding fragment provided herein comprises a HCDR1, a HCDR2, and a HCDR3 as set forth in a VH comprising the amino acid sequence of SEQ ID NO: 297, and a LCDR1, a LCDR2, and a LCDR3 as set forth in a VL comprising the amino acid sequence of SEQ ID NO: 298.

In some embodiments, the antibody or antigen binding fragment provided herein comprises a HCDR1, a HCDR2, and/or a HCDR3 as set forth in a VH comprising the amino acid sequence of SEQ ID NO: 299, and/or a LCDR1, a LCDR2, and/or a LCDR3 as set forth in a VL comprising the amino acid sequence of SEQ ID NO: 300. In some embodiments, the antibody or antigen binding fragment provided herein comprises a HCDR1, a HCDR2, and a HCDR3 as set forth in a VH comprising the amino acid sequence of SEQ ID NO: 299, and a LCDR1, a LCDR2, and a LCDR3 as set forth in a VL comprising the amino acid sequence of SEQ ID NO: 300.

In some embodiments, the antibody or antigen binding fragment provided herein comprises a HCDR1, a HCDR2, and/or a HCDR3 as set forth in a VH comprising the amino acid sequence of SEQ ID NO: 301, and/or a LCDR1, a LCDR2, and/or a LCDR3 as set forth in a VL comprising the amino acid sequence of SEQ ID NO: 302. In some embodiments, the antibody or antigen binding fragment provided herein comprises a HCDR1, a HCDR2, and a HCDR3 as set forth in a VH comprising the amino acid sequence of SEQ ID NO: 301, and a LCDR1, a LCDR2, and a LCDR3 as set forth in a VL comprising the amino acid sequence of SEQ ID NO: 302.

In some embodiments, the antibody or antigen binding fragment provided herein comprises a HCDR1, a HCDR2, and/or a HCDR3 as set forth in a VH comprising the amino acid sequence of SEQ ID NO: 303, and/or a LCDR1, a LCDR2, and/or a LCDR3 as set forth in a VL comprising the amino acid sequence of SEQ ID NO: 304. In some embodiments, the antibody or antigen binding fragment provided herein comprises a HCDR1, a HCDR2, and a HCDR3 as set forth in a VH comprising the amino acid sequence of SEQ ID NO: 303, and a LCDR1, a LCDR2, and a LCDR3 as set forth in a VL comprising the amino acid sequence of SEQ ID NO: 304.

In some embodiments, the antibody or antigen binding fragment provided herein comprises a HCDR1, a HCDR2, and/or a HCDR3 as set forth in a VH comprising the amino acid sequence of SEQ ID NO: 305, and/or a LCDR1, a LCDR2, and/or a LCDR3 as set forth in a VL comprising the amino acid sequence of SEQ ID NO: 306. In some embodiments, the antibody or antigen binding fragment provided herein comprises a HCDR1, a HCDR2, and a HCDR3 as set forth in a VH comprising the amino acid sequence of SEQ ID NO: 305, and a LCDR1, a LCDR2, and a LCDR3 as set forth in a VL comprising the amino acid sequence of SEQ ID NO: 306.

In some embodiments, the antibody or antigen binding fragment provided herein comprises a HCDR1, a HCDR2, and/or a HCDR3 as set forth in a VH comprising the amino acid sequence of SEQ ID NO: 307, and/or a LCDR1, a LCDR2, and/or a LCDR3 as set forth in a VL comprising the amino acid sequence of SEQ ID NO: 308. In some embodiments, the antibody or antigen binding fragment provided herein comprises a HCDR1, a HCDR2, and a HCDR3 as set forth in a VH comprising the amino acid sequence of SEQ ID NO: 307, and a LCDR1, a LCDR2, and a LCDR3 as set forth in a VL comprising the amino acid sequence of SEQ ID NO: 308.

In some embodiments, the antibody or antigen binding fragment provided herein comprises a HCDR1, a HCDR2, and/or a HCDR3 as set forth in a VH comprising the amino acid sequence of SEQ ID NO: 309, and/or a LCDR1, a LCDR2, and/or a LCDR3 as set forth in a VL comprising the amino acid sequence of SEQ ID NO: 310. In some embodiments, the antibody or antigen binding fragment provided herein comprises a HCDR1, a HCDR2, and a HCDR3 as set forth in a VH comprising the amino acid sequence of SEQ ID NO: 309, and a LCDR1, a LCDR2, and a LCDR3 as set forth in a VL comprising the amino acid sequence of SEQ ID NO: 310.

In some embodiments, the antibody or antigen binding fragment provided herein comprises a HCDR1, a HCDR2, and/or a HCDR3 as set forth in a VH comprising the amino acid sequence of SEQ ID NO: 311, and/or a LCDR1, a LCDR2, and/or a LCDR3 as set forth in a VL comprising the amino acid sequence of SEQ ID NO: 312. In some embodiments, the antibody or antigen binding fragment provided herein comprises a HCDR1, a HCDR2, and a HCDR3 as set forth in a VH comprising the amino acid sequence of SEQ ID NO: 311, and a LCDR1, a LCDR2, and a LCDR3 as set forth in a VL comprising the amino acid sequence of SEQ ID NO: 312.

In some embodiments, the antibody or antigen binding fragment provided herein sequence of SEQ ID NO: 313, and/or a LCDR1, a LCDR2, and/or a LCDR3 as set forth in a VL comprising the amino acid sequence of SEQ ID NO: 314. In some embodiments, the antibody or antigen binding fragment provided herein comprises a HCDR1, a HCDR2, and a HCDR3 as set forth in a VH comprising the amino acid sequence of SEQ ID NO: 313, and a LCDR1, a LCDR2, and a LCDR3 as set forth in a VL comprising the amino acid sequence of SEQ ID NO: 314.

In some embodiments, the antibody or antigen binding fragment provided herein comprises a HCDR1, a HCDR2, and/or a HCDR3 as set forth in a VH comprising the amino acid sequence of SEQ ID NO: 315, and/or a LCDR1, a LCDR2, and/or a LCDR3 as set forth in a VL comprising the amino acid sequence of SEQ ID NO: 316. In some embodiments, the antibody or antigen binding fragment provided herein comprises a HCDR1, a HCDR2, and a HCDR3 as set forth in a VH comprising the amino acid sequence of SEQ ID NO: 315, and a LCDR1, a LCDR2, and a LCDR3 as set forth in a VL comprising the amino acid sequence of SEQ ID NO: 316.

In some embodiments, the antibody or antigen binding fragment provided herein comprises a HCDR1, a HCDR2, and/or a HCDR3 as set forth in a VH comprising the amino acid sequence of SEQ ID NO: 317, and/or a LCDR1, a LCDR2, and/or a LCDR3 as set forth in a VL comprising the amino acid sequence of SEQ ID NO: 318. In some embodiments, the antibody or antigen binding fragment provided herein comprises a HCDR1, a HCDR2, and a HCDR3 as set forth in a VH comprising the amino acid sequence of SEQ ID NO: 317, and a LCDR1, a LCDR2, and a LCDR3 as set forth in a VL comprising the amino acid sequence of SEQ ID NO: 318.

In some embodiments, the antibody or antigen binding fragment provided herein comprises a HCDR1, a HCDR2, and/or a HCDR3 as set forth in a VH comprising the amino acid sequence of SEQ ID NO: 319, and/or a LCDR1, a LCDR2, and/or a LCDR3 as set forth in a VL comprising the amino acid sequence of SEQ ID NO: 320. In some embodiments, the antibody or antigen binding fragment provided herein comprises a HCDR1, a HCDR2, and a HCDR3 as set forth in a VH comprising the amino acid sequence of SEQ ID NO: 319, and a LCDR1, a LCDR2, and a LCDR3 as set forth in a VL comprising the amino acid sequence of SEQ ID NO: 320.

In some embodiments, the antibody or antigen binding fragment provided herein comprises a HCDR1, a HCDR2, and/or a HCDR3 as set forth in a VH comprising the amino acid sequence of SEQ ID NO: 321, and/or a LCDR1, a LCDR2, and/or a LCDR3 as set forth in a VL comprising the amino acid sequence of SEQ ID NO: 322. In some embodiments, the antibody or antigen binding fragment provided herein comprises a HCDR1, a HCDR2, and a HCDR3 as set forth in a VH comprising the amino acid sequence of SEQ ID NO: 321, and a LCDR1, a LCDR2, and a LCDR3 as set forth in a VL comprising the amino acid sequence of SEQ ID NO: 322.

In some embodiments, the antibody or antigen binding fragment provided herein comprises a HCDR1, a HCDR2, and/or a HCDR3 as set forth in a VH comprising the amino acid sequence of SEQ ID NO: 323, and/or a LCDR1, a LCDR2, and/or a LCDR3 as set forth in a VL comprising the amino acid sequence of SEQ ID NO: 324. In some embodiments, the antibody or antigen binding fragment provided herein comprises a HCDR1, a HCDR2, and a HCDR3 as set forth in a VH comprising the amino acid sequence of SEQ ID NO: 323, and a LCDR1, a LCDR2, and a LCDR3 as set forth in a VL comprising the amino acid sequence of SEQ ID NO: 324.

In some embodiments, the antibody or antigen binding fragment provided herein comprises a HCDR1, a HCDR2, and/or a HCDR3 as set forth in a VH comprising the amino acid sequence of SEQ ID NO: 325, and/or a LCDR1, a LCDR2, and/or a LCDR3 as set forth in a VL comprising the amino acid sequence of SEQ ID NO: 326. In some embodiments, the antibody or antigen binding fragment provided herein comprises a HCDR1, a HCDR2, and a HCDR3 as set forth in a VH comprising the amino acid sequence of SEQ ID NO: 325, and a LCDR1, a LCDR2, and a LCDR3 as set forth in a VL comprising the amino acid sequence of SEQ ID NO: 326.

In some embodiments, the antibody or antigen binding fragment provided herein comprises a HCDR1, a HCDR2, and/or a HCDR3 as set forth in a VH comprising the amino acid sequence of SEQ ID NO: 327, and/or a LCDR1, a LCDR2, and/or a LCDR3 as set forth in a VL comprising the amino acid sequence of SEQ ID NO: 328. In some embodiments, the antibody or antigen binding fragment provided herein comprises a HCDR1, a HCDR2, and a HCDR3 as set forth in a VH comprising the amino acid sequence of SEQ ID NO: 327, and a LCDR1, a LCDR2, and a LCDR3 as set forth in a VL comprising the amino acid sequence of SEQ ID NO: 328.

In some embodiments, the antibody or antigen binding fragment provided herein comprises a HCDR1, a HCDR2, and/or a HCDR3 as set forth in a VH comprising the amino acid sequence of SEQ ID NO: 329, and/or a LCDR1, a LCDR2, and/or a LCDR3 as set forth in a VL comprising the amino acid sequence of SEQ ID NO: 330. In some embodiments, the antibody or antigen binding fragment provided herein comprises a HCDR1, a HCDR2, and a HCDR3 as set forth in a VH comprising the amino acid sequence of SEQ ID NO: 329, and a LCDR1, a LCDR2, and a LCDR3 as set forth in a VL comprising the amino acid sequence of SEQ ID NO: 330.

In some embodiments, the antibody or antigen binding fragment provided herein sequence of SEQ ID NO: 331, and/or a LCDR1, a LCDR2, and/or a LCDR3 as set forth in a VL comprising the amino acid sequence of SEQ ID NO: 332. In some embodiments, the antibody or antigen binding fragment provided herein comprises a HCDR1, a HCDR2, and a HCDR3 as set forth in a VH comprising the amino acid sequence of SEQ ID NO: 331, and a LCDR1, a LCDR2, and a LCDR3 as set forth in a VL comprising the amino acid sequence of SEQ ID NO: 332.

In some embodiments, the antibody or antigen binding fragment provided herein comprises a HCDR1, a HCDR2, and/or a HCDR3 as set forth in a VH comprising the amino acid sequence of SEQ ID NO: 333, and/or a LCDR1, a LCDR2, and/or a LCDR3 as set forth in a VL comprising the amino acid sequence of SEQ ID NO: 334. In some embodiments, the antibody or antigen binding fragment provided herein comprises a HCDR1, a HCDR2, and a HCDR3 as set forth in a VH comprising the amino acid sequence of SEQ ID NO: 333, and a LCDR1, a LCDR2, and a LCDR3 as set forth in a VL comprising the amino acid sequence of SEQ ID NO: 334.

In some embodiments, the antibody or antigen binding fragment provided herein comprises a HCDR1, a HCDR2, and/or a HCDR3 as set forth in a VH comprising the amino acid sequence of SEQ ID NO: 335, and/or a LCDR1, a LCDR2, and/or a LCDR3 as set forth in a VL comprising the amino acid sequence of SEQ ID NO: 336. In some embodiments, the antibody or antigen binding fragment provided herein comprises a HCDR1, a HCDR2, and a HCDR3 as set forth in a VH comprising the amino acid sequence of SEQ ID NO: 335, and a LCDR1, a LCDR2, and a LCDR3 as set forth in a VL comprising the amino acid sequence of SEQ ID NO: 336.

In some embodiments, the antibody or antigen binding fragment provided herein comprises a HCDR1, a HCDR2, and/or a HCDR3 as set forth in a VH comprising the amino acid sequence of SEQ ID NO: 337, and/or a LCDR1, a LCDR2, and/or a LCDR3 as set forth in a VL comprising the amino acid sequence of SEQ ID NO: 338. In some embodiments, the antibody or antigen binding fragment provided herein comprises a HCDR1, a HCDR2, and a HCDR3 as set forth in a VH comprising the amino acid sequence of SEQ ID NO: 337, and a LCDR1, a LCDR2, and a LCDR3 as set forth in a VL comprising the amino acid sequence of SEQ ID NO: 338.

In some embodiments, the antibody or antigen binding fragment provided herein comprises a HCDR1, a HCDR2, and/or a HCDR3 as set forth in a VH comprising the amino acid sequence of SEQ ID NO: 339, and/or a LCDR1, a LCDR2, and/or a LCDR3 as set forth in a VL comprising the amino acid sequence of SEQ ID NO: 340. In some embodiments, the antibody or antigen binding fragment provided herein comprises a HCDR1, a HCDR2, and a HCDR3 as set forth in a VH comprising the amino acid sequence of SEQ ID NO: 339, and a LCDR1, a LCDR2, and a LCDR3 as set forth in a VL comprising the amino acid sequence of SEQ ID NO: 340.

In some embodiments, the antibody or antigen binding fragment provided herein comprises a HCDR1, a HCDR2, and/or a HCDR3 as set forth in a VH comprising the amino acid sequence of SEQ ID NO: 341, and/or a LCDR1, a LCDR2, and/or a LCDR3 as set forth in a VL comprising the amino acid sequence of SEQ ID NO: 342. In some embodiments, the antibody or antigen binding fragment provided herein comprises a HCDR1, a HCDR2, and a HCDR3 as set forth in a VH comprising the amino acid sequence of SEQ ID NO: 341, and a LCDR1, a LCDR2, and a LCDR3 as set forth in a VL comprising the amino acid sequence of SEQ ID NO: 342.

In some embodiments, the antibody or antigen binding fragment provided herein comprises a HCDR1, a HCDR2, and/or a HCDR3 as set forth in a VH comprising the amino acid sequence of SEQ ID NO: 343, and/or a LCDR1, a LCDR2, and/or a LCDR3 as set forth in a VL comprising the amino acid sequence of SEQ ID NO: 344. In some embodiments, the antibody or antigen binding fragment provided herein comprises a HCDR1, a HCDR2, and a HCDR3 as set forth in a VH comprising the amino acid sequence of SEQ ID NO: 343, and a LCDR1, a LCDR2, and a LCDR3 as set forth in a VL comprising the amino acid sequence of SEQ ID NO: 344.

In some embodiments, the antibody or antigen binding fragment provided herein comprises a HCDR1, a HCDR2, and/or a HCDR3 as set forth in a VH comprising the amino acid sequence of SEQ ID NO: 345, and/or a LCDR1, a LCDR2, and/or a LCDR3 as set forth in a VL comprising the amino acid sequence of SEQ ID NO: 346. In some embodiments, the antibody or antigen binding fragment provided herein comprises a HCDR1, a HCDR2, and a HCDR3 as set forth in a VH comprising the amino acid sequence of SEQ ID NO: 345, and a LCDR1, a LCDR2, and a LCDR3 as set forth in a VL comprising the amino acid sequence of SEQ ID NO: 346.

In some embodiments, the antibody or antigen binding fragment provided herein comprises a HCDR1, a HCDR2, and/or a HCDR3 as set forth in a VH comprising the amino acid sequence of SEQ ID NO: 347, and/or a LCDR1, a LCDR2, and/or a LCDR3 as set forth in a VL comprising the amino acid sequence of SEQ ID NO: 348. In some embodiments, the antibody or antigen binding fragment provided herein comprises a HCDR1, a HCDR2, and a HCDR3 as set forth in a VH comprising the amino acid sequence of SEQ ID NO: 347, and a LCDR1, a LCDR2, and a LCDR3 as set forth in a VL comprising the amino acid sequence of SEQ ID NO: 348.

In some embodiments, the antibody or antigen binding fragment provided herein sequence of SEQ ID NO: 349, and/or a LCDR1, a LCDR2, and/or a LCDR3 as set forth in a VL comprising the amino acid sequence of SEQ ID NO: 350. In some embodiments, the antibody or antigen binding fragment provided herein comprises a HCDR1, a HCDR2, and a HCDR3 as set forth in a VH comprising the amino acid sequence of SEQ ID NO: 349, and a LCDR1, a LCDR2, and a LCDR3 as set forth in a VL comprising the amino acid sequence of SEQ ID NO: 350.

In some embodiments, the antibody or antigen binding fragment provided herein comprises a HCDR1, a HCDR2, and/or a HCDR3 as set forth in a VH comprising the amino acid sequence of SEQ ID NO: 351, and/or a LCDR1, a LCDR2, and/or a LCDR3 as set forth in a VL comprising the amino acid sequence of SEQ ID NO: 352. In some embodiments, the antibody or antigen binding fragment provided herein comprises a HCDR1, a HCDR2, and a HCDR3 as set forth in a VH comprising the amino acid sequence of SEQ ID NO: 351, and a LCDR1, a LCDR2, and a LCDR3 as set forth in a VL comprising the amino acid sequence of SEQ ID NO: 352.

In some embodiments, the antibody or antigen binding fragment provided herein comprises a HCDR1, a HCDR2, and/or a HCDR3 as set forth in a VH comprising the amino acid sequence of SEQ ID NO: 353, and/or a LCDR1, a LCDR2, and/or a LCDR3 as set forth in a VL comprising the amino acid sequence of SEQ ID NO: 354. In some embodiments, the antibody or antigen binding fragment provided herein comprises a HCDR1, a HCDR2, and a HCDR3 as set forth in a VH comprising the amino acid sequence of SEQ ID NO: 353, and a LCDR1, a LCDR2, and a LCDR3 as set forth in a VL comprising the amino acid sequence of SEQ ID NO: 354.

In some embodiments, the antibody or antigen binding fragment provided herein comprises a HCDR1, a HCDR2, and/or a HCDR3 as set forth in a VH comprising the amino acid sequence of SEQ ID NO: 355, and/or a LCDR1, a LCDR2, and/or a LCDR3 as set forth in a VL comprising the amino acid sequence of SEQ ID NO: 356. In some embodiments, the antibody or antigen binding fragment provided herein comprises a HCDR1, a HCDR2, and a HCDR3 as set forth in a VH comprising the amino acid sequence of SEQ ID NO: 355, and a LCDR1, a LCDR2, and a LCDR3 as set forth in a VL comprising the amino acid sequence of SEQ ID NO: 356.

In some embodiments, the antibody or antigen binding fragment provided herein comprises a HCDR1, a HCDR2, and/or a HCDR3 as set forth in a VH comprising the amino acid sequence of SEQ ID NO: 357, and/or a LCDR1, a LCDR2, and/or a LCDR3 as set forth in a VL comprising the amino acid sequence of SEQ ID NO: 358. In some embodiments, the antibody or antigen binding fragment provided herein comprises a HCDR1, a HCDR2, and a HCDR3 as set forth in a VH comprising the amino acid sequence of SEQ ID NO: 357, and a LCDR1, a LCDR2, and a LCDR3 as set forth in a VL comprising the amino acid sequence of SEQ ID NO: 358.

In some embodiments, the antibody or antigen binding fragment provided herein comprises a HCDR1, a HCDR2, and/or a HCDR3 as set forth in a VH comprising the amino acid sequence of SEQ ID NO: 359, and/or a LCDR1, a LCDR2, and/or a LCDR3 as set forth in a VL comprising the amino acid sequence of SEQ ID NO: 360. In some embodiments, the antibody or antigen binding fragment provided herein comprises a HCDR1, a HCDR2, and a HCDR3 as set forth in a VH comprising the amino acid sequence of SEQ ID NO: 359, and a LCDR1, a LCDR2, and a LCDR3 as set forth in a VL comprising the amino acid sequence of SEQ ID NO: 360.

In some embodiments, the antibody or antigen binding fragment provided herein comprises a HCDR1, a HCDR2, and/or a HCDR3 as set forth in a VH comprising the amino acid sequence of SEQ ID NO: 361, and/or a LCDR1, a LCDR2, and/or a LCDR3 as set forth in a VL comprising the amino acid sequence of SEQ ID NO: 362. In some embodiments, the antibody or antigen binding fragment provided herein comprises a HCDR1, a HCDR2, and a HCDR3 as set forth in a VH comprising the amino acid sequence of SEQ ID NO: 361, and a LCDR1, a LCDR2, and a LCDR3 as set forth in a VL comprising the amino acid sequence of SEQ ID NO: 362.

In some embodiments, the antibody or antigen binding fragment provided herein comprises a HCDR1, a HCDR2, and/or a HCDR3 as set forth in a VH comprising the amino acid sequence of SEQ ID NO: 363, and/or a LCDR1, a LCDR2, and/or a LCDR3 as set forth in a VL comprising the amino acid sequence of SEQ ID NO: 364. In some embodiments, the antibody or antigen binding fragment provided herein comprises a HCDR1, a HCDR2, and a HCDR3 as set forth in a VH comprising the amino acid sequence of SEQ ID NO: 363, and a LCDR1, a LCDR2, and a LCDR3 as set forth in a VL comprising the amino acid sequence of SEQ ID NO: 364.

In some embodiments, the antibody or antigen binding fragment provided herein comprises a HCDR1, a HCDR2, and/or a HCDR3 as set forth in a VH comprising the amino acid sequence of SEQ ID NO: 365, and/or a LCDR1, a LCDR2, and/or a LCDR3 as set forth in a VL comprising the amino acid sequence of SEQ ID NO: 366. In some embodiments, the antibody or antigen binding fragment provided herein comprises a HCDR1, a HCDR2, and a HCDR3 as set forth in a VH comprising the amino acid sequence of SEQ ID NO: 365, and a LCDR1, a LCDR2, and a LCDR3 as set forth in a VL comprising the amino acid sequence of SEQ ID NO: 366.

In some embodiments, the antibody or antigen binding fragment provided herein sequence of SEQ ID NO: 367, and/or a LCDR1, a LCDR2, and/or a LCDR3 as set forth in a VL comprising the amino acid sequence of SEQ ID NO: 368. In some embodiments, the antibody or antigen binding fragment provided herein comprises a HCDR1, a HCDR2, and a HCDR3 as set forth in a VH comprising the amino acid sequence of SEQ ID NO: 367, and a LCDR1, a LCDR2, and a LCDR3 as set forth in a VL comprising the amino acid sequence of SEQ ID NO: 368.

In some embodiments, the antibody or antigen binding fragment provided herein comprises a HCDR1, a HCDR2, and/or a HCDR3 as set forth in a VH comprising the amino acid sequence of SEQ ID NO: 369, and/or a LCDR1, a LCDR2, and/or a LCDR3 as set forth in a VL comprising the amino acid sequence of SEQ ID NO: 370. In some embodiments, the antibody or antigen binding fragment provided herein comprises a HCDR1, a HCDR2, and a HCDR3 as set forth in a VH comprising the amino acid sequence of SEQ ID NO: 369, and a LCDR1, a LCDR2, and a LCDR3 as set forth in a VL comprising the amino acid sequence of SEQ ID NO: 370.

In some embodiments, the antibody or antigen binding fragment provided herein comprises a HCDR1, a HCDR2, and/or a HCDR3 as set forth in a VH comprising the amino acid sequence of SEQ ID NO: 371, and/or a LCDR1, a LCDR2, and/or a LCDR3 as set forth in a VL comprising the amino acid sequence of SEQ ID NO: 372. In some embodiments, the antibody or antigen binding fragment provided herein comprises a HCDR1, a HCDR2, and a HCDR3 as set forth in a VH comprising the amino acid sequence of SEQ ID NO: 371, and a LCDR1, a LCDR2, and a LCDR3 as set forth in a VL comprising the amino acid sequence of SEQ ID NO: 372.

In some embodiments, the antibody or antigen binding fragment provided herein comprises a HCDR1, a HCDR2, and/or a HCDR3 as set forth in a VH comprising the amino acid sequence of SEQ ID NO: 373, and/or a LCDR1, a LCDR2, and/or a LCDR3 as set forth in a VL comprising the amino acid sequence of SEQ ID NO: 374. In some embodiments, the antibody or antigen binding fragment provided herein comprises a HCDR1, a HCDR2, and a HCDR3 as set forth in a VH comprising the amino acid sequence of SEQ ID NO: 373, and a LCDR1, a LCDR2, and a LCDR3 as set forth in a VL comprising the amino acid sequence of SEQ ID NO: 374.

In some embodiments, the antibody or antigen binding fragment provided herein comprises a HCDR1, a HCDR2, and/or a HCDR3 as set forth in a VH comprising the amino acid sequence of SEQ ID NO: 375, and/or a LCDR1, a LCDR2, and/or a LCDR3 as set forth in a VL comprising the amino acid sequence of SEQ ID NO: 376. In some embodiments, the antibody or antigen binding fragment provided herein comprises a HCDR1, a HCDR2, and a HCDR3 as set forth in a VH comprising the amino acid sequence of SEQ ID NO: 375, and a LCDR1, a LCDR2, and a LCDR3 as set forth in a VL comprising the amino acid sequence of SEQ ID NO: 376.

In some embodiments, the antibody or antigen binding fragment provided herein comprises a HCDR1, a HCDR2, and/or a HCDR3 as set forth in a VH comprising the amino acid sequence of SEQ ID NO: 377, and/or a LCDR1, a LCDR2, and/or a LCDR3 as set forth in a VL comprising the amino acid sequence of SEQ ID NO: 378. In some embodiments, the antibody or antigen binding fragment provided herein comprises a HCDR1, a HCDR2, and a HCDR3 as set forth in a VH comprising the amino acid sequence of SEQ ID NO: 377, and a LCDR1, a LCDR2, and a LCDR3 as set forth in a VL comprising the amino acid sequence of SEQ ID NO: 378.

In some embodiments, the antibody or antigen binding fragment provided herein comprises a HCDR1, a HCDR2, and/or a HCDR3 as set forth in a VH comprising the amino acid sequence of SEQ ID NO: 379, and/or a LCDR1, a LCDR2, and/or a LCDR3 as set forth in a VL comprising the amino acid sequence of SEQ ID NO: 380. In some embodiments, the antibody or antigen binding fragment provided herein comprises a HCDR1, a HCDR2, and a HCDR3 as set forth in a VH comprising the amino acid sequence of SEQ ID NO: 379, and a LCDR1, a LCDR2, and a LCDR3 as set forth in a VL comprising the amino acid sequence of SEQ ID NO: 380.

In some embodiments, the antibody or antigen binding fragment provided herein comprises a HCDR1, a HCDR2, and/or a HCDR3 as set forth in a VH comprising the amino acid sequence of SEQ ID NO: 381, and/or a LCDR1, a LCDR2, and/or a LCDR3 as set forth in a VL comprising the amino acid sequence of SEQ ID NO: 382. In some embodiments, the antibody or antigen binding fragment provided herein comprises a HCDR1, a HCDR2, and a HCDR3 as set forth in a VH comprising the amino acid sequence of SEQ ID NO: 381, and a LCDR1, a LCDR2, and a LCDR3 as set forth in a VL comprising the amino acid sequence of SEQ ID NO: 382.

In some embodiments, the antibody or antigen binding fragment provided herein comprises a HCDR1, a HCDR2, and/or a HCDR3 as set forth in a VH comprising the amino acid sequence of SEQ ID NO: 383, and/or a LCDR1, a LCDR2, and/or a LCDR3 as set forth in a VL comprising the amino acid sequence of SEQ ID NO: 384. In some embodiments, the antibody or antigen binding fragment provided herein comprises a HCDR1, a HCDR2, and a HCDR3 as set forth in a VH comprising the amino acid sequence of SEQ ID NO: 383, and a LCDR1, a LCDR2, and a LCDR3 as set forth in a VL comprising the amino acid sequence of SEQ ID NO: 384.

In some embodiments, the antibody or antigen binding fragment provided herein sequence of SEQ ID NO: 385, and/or a LCDR1, a LCDR2, and/or a LCDR3 as set forth in a VL comprising the amino acid sequence of SEQ ID NO: 386. In some embodiments, the antibody or antigen binding fragment provided herein comprises a HCDR1, a HCDR2, and a HCDR3 as set forth in a VH comprising the amino acid sequence of SEQ ID NO: 385, and a LCDR1, a LCDR2, and a LCDR3 as set forth in a VL comprising the amino acid sequence of SEQ ID NO: 386.

In some embodiments, the antibody or antigen binding fragment provided herein comprises a HCDR1, a HCDR2, and/or a HCDR3 as set forth in a VH comprising the amino acid sequence of SEQ ID NO: 387, and/or a LCDR1, a LCDR2, and/or a LCDR3 as set forth in a VL comprising the amino acid sequence of SEQ ID NO: 388. In some embodiments, the antibody or antigen binding fragment provided herein comprises a HCDR1, a HCDR2, and a HCDR3 as set forth in a VH comprising the amino acid sequence of SEQ ID NO: 387, and a LCDR1, a LCDR2, and a LCDR3 as set forth in a VL comprising the amino acid sequence of SEQ ID NO: 388.

In some embodiments, the antibody or antigen binding fragment provided herein comprises a HCDR1, a HCDR2, and/or a HCDR3 as set forth in a VH comprising the amino acid sequence of SEQ ID NO: 389, and/or a LCDR1, a LCDR2, and/or a LCDR3 as set forth in a VL comprising the amino acid sequence of SEQ ID NO: 390. In some embodiments, the antibody or antigen binding fragment provided herein comprises a HCDR1, a HCDR2, and a HCDR3 as set forth in a VH comprising the amino acid sequence of SEQ ID NO: 389, and a LCDR1, a LCDR2, and a LCDR3 as set forth in a VL comprising the amino acid sequence of SEQ ID NO: 390.

In some embodiments, the antibody or antigen binding fragment provided herein comprises a HCDR1, a HCDR2, and/or a HCDR3 as set forth in a VH comprising the amino acid sequence of SEQ ID NO: 391, and/or a LCDR1, a LCDR2, and/or a LCDR3 as set forth in a VL comprising the amino acid sequence of SEQ ID NO: 392. In some embodiments, the antibody or antigen binding fragment provided herein comprises a HCDR1, a HCDR2, and a HCDR3 as set forth in a VH comprising the amino acid sequence of SEQ ID NO: 391, and a LCDR1, a LCDR2, and a LCDR3 as set forth in a VL comprising the amino acid sequence of SEQ ID NO: 392.

In some embodiments, the antibody or antigen binding fragment provided herein comprises a HCDR1, a HCDR2, and/or a HCDR3 as set forth in a VH comprising the amino acid sequence of SEQ ID NO: 393, and/or a LCDR1, a LCDR2, and/or a LCDR3 as set forth in a VL comprising the amino acid sequence of SEQ ID NO: 394. In some embodiments, the antibody or antigen binding fragment provided herein comprises a HCDR1, a HCDR2, and a HCDR3 as set forth in a VH comprising the amino acid sequence of SEQ ID NO: 393, and a LCDR1, a LCDR2, and a LCDR3 as set forth in a VL comprising the amino acid sequence of SEQ ID NO: 394.

In some embodiments, the antibody or antigen binding fragment provided herein comprises a HCDR1, a HCDR2, and/or a HCDR3 as set forth in a VH comprising the amino acid sequence of SEQ ID NO: 395, and/or a LCDR1, a LCDR2, and/or a LCDR3 as set forth in a VL comprising the amino acid sequence of SEQ ID NO: 396. In some embodiments, the antibody or antigen binding fragment provided herein comprises a HCDR1, a HCDR2, and a HCDR3 as set forth in a VH comprising the amino acid sequence of SEQ ID NO: 395, and a LCDR1, a LCDR2, and a LCDR3 as set forth in a VL comprising the amino acid sequence of SEQ ID NO: 396.

In some embodiments, the antibody or antigen binding fragment provided herein comprises a HCDR1, a HCDR2, and/or a HCDR3 as set forth in a VH comprising the amino acid sequence of SEQ ID NO: 397, and/or a LCDR1, a LCDR2, and/or a LCDR3 as set forth in a VL comprising the amino acid sequence of SEQ ID NO: 398. In some embodiments, the antibody or antigen binding fragment provided herein comprises a HCDR1, a HCDR2, and a HCDR3 as set forth in a VH comprising the amino acid sequence of SEQ ID NO: 397, and a LCDR1, a LCDR2, and a LCDR3 as set forth in a VL comprising the amino acid sequence of SEQ ID NO: 398.

In some embodiments, the antibody or antigen binding fragment provided herein comprises a HCDR1, a HCDR2, and/or a HCDR3 as set forth in a VH comprising the amino acid sequence of SEQ ID NO: 399, and/or a LCDR1, a LCDR2, and/or a LCDR3 as set forth in a VL comprising the amino acid sequence of SEQ ID NO: 400. In some embodiments, the antibody or antigen binding fragment provided herein comprises a HCDR1, a HCDR2, and a HCDR3 as set forth in a VH comprising the amino acid sequence of SEQ ID NO: 399, and a LCDR1, a LCDR2, and a LCDR3 as set forth in a VL comprising the amino acid sequence of SEQ ID NO: 400.

In some embodiments, the antibody or antigen binding fragment provided herein comprises a HCDR1, a HCDR2, and/or a HCDR3 as set forth in a VH comprising the amino acid sequence of SEQ ID NO: 1155, and/or a LCDR1, a LCDR2, and/or a LCDR3 as set forth in a VL comprising the amino acid sequence of SEQ ID NO: 1156. In some embodiments, the antibody or antigen binding fragment provided herein comprises a HCDR1, a HCDR2, and a HCDR3 as set forth in a VH comprising the amino acid sequence of SEQ ID NO: 1155, and a LCDR1, a LCDR2, and a LCDR3 as set forth in a VL comprising the amino acid sequence of SEQ ID NO: 1156.

In some embodiments, the antibody or antigen binding fragment provided herein sequence of SEQ ID NO: 1157, and/or a LCDR1, a LCDR2, and/or a LCDR3 as set forth in a VL comprising the amino acid sequence of SEQ ID NO: 1158. In some embodiments, the antibody or antigen binding fragment provided herein comprises a HCDR1, a HCDR2, and a HCDR3 as set forth in a VH comprising the amino acid sequence of SEQ ID NO: 1157, and a LCDR1, a LCDR2, and a LCDR3 as set forth in a VL comprising the amino acid sequence of SEQ ID NO: 1158.

In some embodiments, the antibody or antigen binding fragment provided herein comprises a HCDR1, a HCDR2, and/or a HCDR3 as set forth in a VH comprising the amino acid sequence of SEQ ID NO: 1159, and/or a LCDR1, a LCDR2, and/or a LCDR3 as set forth in a VL comprising the amino acid sequence of SEQ ID NO: 1160. In some embodiments, the antibody or antigen binding fragment provided herein comprises a HCDR1, a HCDR2, and a HCDR3 as set forth in a VH comprising the amino acid sequence of SEQ ID NO: 1159, and a LCDR1, a LCDR2, and a LCDR3 as set forth in a VL comprising the amino acid sequence of SEQ ID NO: 1160.

In some embodiments, the antibody or antigen binding fragment provided herein comprises a HCDR1, a HCDR2, and/or a HCDR3 as set forth in a VH comprising the amino acid sequence of SEQ ID NO: 1161, and/or a LCDR1, a LCDR2, and/or a LCDR3 as set forth in a VL comprising the amino acid sequence of SEQ ID NO: 1162. In some embodiments, the antibody or antigen binding fragment provided herein comprises a HCDR1, a HCDR2, and a HCDR3 as set forth in a VH comprising the amino acid sequence of SEQ ID NO: 1161, and a LCDR1, a LCDR2, and a LCDR3 as set forth in a VL comprising the amino acid sequence of SEQ ID NO: 1162.

In some embodiments, the antibody or antigen binding fragment provided herein comprises a HCDR1, a HCDR2, and/or a HCDR3 as set forth in a VH comprising the amino acid sequence of SEQ ID NO: 1163, and/or a LCDR1, a LCDR2, and/or a LCDR3 as set forth in a VL comprising the amino acid sequence of SEQ ID NO: 1164. In some embodiments, the antibody or antigen binding fragment provided herein comprises a HCDR1, a HCDR2, and a HCDR3 as set forth in a VH comprising the amino acid sequence of SEQ ID NO: 1163, and a LCDR1, a LCDR2, and a LCDR3 as set forth in a VL comprising the amino acid sequence of SEQ ID NO: 1164.

In some embodiments, the antibody or antigen binding fragment provided herein comprises a HCDR1, a HCDR2, and/or a HCDR3 as set forth in a VH comprising the amino acid sequence of SEQ ID NO: 1165, and/or a LCDR1, a LCDR2, and/or a LCDR3 as set forth in a VL comprising the amino acid sequence of SEQ ID NO: 1166. In some embodiments, the antibody or antigen binding fragment provided herein comprises a HCDR1, a HCDR2, and a HCDR3 as set forth in a VH comprising the amino acid sequence of SEQ ID NO: 1165, and a LCDR1, a LCDR2, and a LCDR3 as set forth in a VL comprising the amino acid sequence of SEQ ID NO: 1166.

CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering.

In other embodiments, provided herein is an antibody that binds to LILRB1 comprising (a) a VH region comprising (i) a HCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NOs: 495, 501, 507, 513, 519, 525, 531, 537, 543, 549, 555, 561, 567, 573, 579, 585, 591, 597, 603, 609, 615, 621, 627, 633, 639, 645, 651, 657, 663, 669, 675, 681, 687, 693, 699, 705, 711, 717, 723, 729, 735, 741, 747, 753, 759, 765, 771, 777, 783, 789, 795, 801, 807, 813, 819, 825, 831, 837, and 843; (ii) a HCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NOs: 496, 502, 508, 514, 520, 526, 532, 538, 544, 550, 556, 562, 568, 574, 580, 586, 592, 598, 604, 610, 616, 622, 628, 634, 640, 646, 652, 658, 664, 670, 676, 682, 688, 694, 700, 706, 712, 718, 724, 730, 736, 742, 748, 754, 760, 766, 772, 778, 784, 790, 796, 802, 808, 814, 820, 826, 832, 838, and 844, (iii) a HCDR3 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 497, 503, 509, 515, 521, 527, 533, 539, 545, 551, 557, 563, 569, 575, 581, 587, 593, 599, 605, 611, 617, 623, 629, 635, 641, 647, 653, 659, 665, 671, 677, 683, 689, 695, 701, 707, 713, 719, 725, 731, 737, 743, 749, 755, 761, 767, 773, 779, 785, 791, 797, 803, 809, 815, 821, 827, 833, 839, 845, and 1179; and/or (b) a VL region comprising (i) a LCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NOs: 498, 504, 510, 516, 522, 528, 534, 540, 546, 552, 558, 564, 570, 576, 582, 588, 594, 600, 606, 612, 618, 624, 630, 636, 642, 648, 654, 660, 666, 672, 678, 684, 690, 696, 702, 708, 714, 720, 726, 732, 738, 744, 750, 756, 762, 768, 774, 780, 786, 792, 798, 804, 810, 816, 822, 828, 834, 840, and 846; (ii) a LCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NOs: 499, 505, 511, 517, 523, 529, 535, 541, 547, 553, 559, 565, 571, 577, 583, 589, 595, 601, 607, 613, 619, 625, 631, 637, 643, 649, 655, 661, 667, 673, 679, 685, 691, 697, 703, 709, 715, 721, 727, 733, 739, 745, 751, 757, 763, 769, 775, 781, 787, 793, 799, 805, 811, 817, 823, 829, 835, 841, 847, and 1281; and (iii) a LCDR3 comprising an amino acid sequence having at least 75%, 80%, 85%, 86%, 87%, 88%, 89%, 90%, 91%, 92%, 93%, 94%, 95%, 96%, 97%, 98%, 99%, 100% sequence identity to any of SEQ ID NOs: 500, 506, 512, 518, 524, 530, 536, 542, 548, 554, 560, 566, 572, 578, 584, 590, 596, 602, 608, 614, 620, 626, 632, 638, 644, 650, 656, 662, 668, 674, 680, 686, 692, 698, 704, 710, 716, 722, 728, 734, 740, 746, 752, 758, 764, 770, 776, 782, 788, 794, 800, 806, 812, 818, 824, 830, 836, 842, and 848. In some embodiments, the anti-LILRB1 antibody is humanized. In some embodiments, the anti-LILRB1 antibody comprises an acceptor human framework, e.g., a human immunoglobulin framework or a human consensus framework.

In some specific embodiments, in the antibody or antigen binding fragment provided herein, the HCDR1 comprises the amino acid sequence of SEQ ID NO: 495, the HCDR2 comprises the amino acid sequence of SEQ ID NO: 496, the HCDR3 comprises the amino acid sequence of SEQ ID NO: 497, the LCDR1 comprises the amino acid sequence of SEQ ID NO: 498, the LCDR2 comprises the amino acid sequence of SEQ ID NO: 499, and the LCDR3 comprises the amino acid sequence of SEQ ID NO: 500.

In some specific embodiments, in the antibody or antigen binding fragment provided herein, the HCDR1 comprises the amino acid sequence of SEQ ID NO: 501, the HCDR2 comprises the amino acid sequence of SEQ ID NO: 502, the HCDR3 comprises the amino acid sequence of SEQ ID NO: 503, the LCDR1 comprises the amino acid sequence of SEQ ID NO: 504, the LCDR2 comprises the amino acid sequence of SEQ ID NO: 505, and the LCDR3 comprises the amino acid sequence of SEQ ID NO: 506.

In some specific embodiments, in the antibody or antigen binding fragment provided herein, the HCDR1 comprises the amino acid sequence of SEQ ID NO: 507, the HCDR2 comprises the amino acid sequence of SEQ ID NO: 508, the HCDR3 comprises the amino acid sequence of SEQ ID NO: 509, the LCDR1 comprises the amino acid sequence of SEQ ID NO: 510, the LCDR2 comprises the amino acid sequence of SEQ ID NO: 511, and the LCDR3 comprises the amino acid sequence of SEQ ID NO: 512.

In some specific embodiments, in the antibody or antigen binding fragment provided herein, the HCDR1 comprises the amino acid sequence of SEQ ID NO: 513, the HCDR2 comprises the amino acid sequence of SEQ ID NO: 514, the HCDR3 comprises the amino acid sequence of SEQ ID NO: 515, the LCDR1 comprises the amino acid sequence of SEQ ID NO: 516, the LCDR2 comprises the amino acid sequence of SEQ ID NO: 517, and the LCDR3 comprises the amino acid sequence of SEQ ID NO: 518.

In some specific embodiments, in the antibody or antigen binding fragment provided herein, the HCDR1 comprises the amino acid sequence of SEQ ID NO: 519, the HCDR2 comprises the amino acid sequence of SEQ ID NO: 520, the HCDR3 comprises the amino acid sequence of SEQ ID NO: 521, the LCDR1 comprises the amino acid sequence of SEQ ID NO: 522, the LCDR2 comprises the amino acid sequence of SEQ ID NO: 523, and the LCDR3 comprises the amino acid sequence of SEQ ID NO: 524.

In some specific embodiments, in the antibody or antigen binding fragment provided herein, the HCDR1 comprises the amino acid sequence of SEQ ID NO: 525, the HCDR2 comprises the amino acid sequence of SEQ ID NO: 526, the HCDR3 comprises the amino acid sequence of SEQ ID NO: 527, the LCDR1 comprises the amino acid sequence of SEQ ID NO: 528, the LCDR2 comprises the amino acid sequence of SEQ ID NO: 529, and the LCDR3 comprises the amino acid sequence of SEQ ID NO: 530.

In some specific embodiments, in the antibody or antigen binding fragment provided herein, the HCDR1 comprises the amino acid sequence of SEQ ID NO: 531, the HCDR2 comprises the amino acid sequence of SEQ ID NO: 532, the HCDR3 comprises the amino acid sequence of SEQ ID NO: 533, the LCDR1 comprises the amino acid sequence of SEQ ID NO: 534, the LCDR2 comprises the amino acid sequence of SEQ ID NO: 535, and the LCDR3 comprises the amino acid sequence of SEQ ID NO: 536.

In some specific embodiments, in the antibody or antigen binding fragment provided herein, the HCDR1 comprises the amino acid sequence of SEQ ID NO: 537, the HCDR2 comprises the amino acid sequence of SEQ ID NO: 538, the HCDR3 comprises the amino acid sequence of SEQ ID NO: 539, the LCDR1 comprises the amino acid sequence of SEQ ID NO: 540, the LCDR2 comprises the amino acid sequence of SEQ ID NO: 541, and the LCDR3 comprises the amino acid sequence of SEQ ID NO: 542.

In some specific embodiments, in the antibody or antigen binding fragment provided herein, the HCDR1 comprises the amino acid sequence of SEQ ID NO: 543, the HCDR2 comprises the amino acid sequence of SEQ ID NO: 544, the HCDR3 comprises the amino acid sequence of SEQ ID NO: 545, the LCDR1 comprises the amino acid sequence of SEQ ID NO: 546, the LCDR2 comprises the amino acid sequence of SEQ ID NO: 547, and the LCDR3 comprises the amino acid sequence of SEQ ID NO: 548.

In some specific embodiments, in the antibody or antigen binding fragment provided herein, the HCDR1 comprises the amino acid sequence of SEQ ID NO: 549, the HCDR2 comprises the amino acid sequence of SEQ ID NO: 550, the HCDR3 comprises the amino acid sequence of SEQ ID NO: 551, the LCDR1 comprises the amino acid sequence of SEQ ID NO: 552, the LCDR2 comprises the amino acid sequence of SEQ ID NO: 553, and the LCDR3 comprises the amino acid sequence of SEQ ID NO: 554.

In some specific embodiments, in the antibody or antigen binding fragment provided herein, the HCDR1 comprises the amino acid sequence of SEQ ID NO: 555, the HCDR2 comprises the amino acid sequence of SEQ ID NO: 556, the HCDR3 comprises the amino acid sequence of SEQ ID NO: 557, the LCDR1 comprises the amino acid sequence of SEQ ID NO: 558, the LCDR2 comprises the amino acid sequence of SEQ ID NO: 559, and the LCDR3 comprises the amino acid sequence of SEQ ID NO: 560.

In some specific embodiments, in the antibody or antigen binding fragment provided herein, the HCDR1 comprises the amino acid sequence of SEQ ID NO: 561, the HCDR2 comprises the amino acid sequence of SEQ ID NO: 562, the HCDR3 comprises the amino acid sequence of SEQ ID NO: 563, the LCDR1 comprises the amino acid sequence of SEQ ID NO: 564, the LCDR2 comprises the amino acid sequence of SEQ ID NO: 565, and the LCDR3 comprises the amino acid sequence of SEQ ID NO: 566.

In some specific embodiments, in the antibody or antigen binding fragment provided herein, the HCDR1 comprises the amino acid sequence of SEQ ID NO: 567, the HCDR2 comprises the amino acid sequence of SEQ ID NO: 568, the HCDR3 comprises the amino acid sequence of SEQ ID NO: 569, the LCDR1 comprises the amino acid sequence of SEQ ID NO: 570, the LCDR2 comprises the amino acid sequence of SEQ ID NO: 571, and the LCDR3 comprises the amino acid sequence of SEQ ID NO: 572.

In some specific embodiments, in the antibody or antigen binding fragment provided herein, the HCDR1 comprises the amino acid sequence of SEQ ID NO: 573, the HCDR2 comprises the amino acid sequence of SEQ ID NO: 574, the HCDR3 comprises the amino acid sequence of SEQ ID NO: 575, the LCDR1 comprises the amino acid sequence of SEQ ID NO: 576, the LCDR2 comprises the amino acid sequence of SEQ ID NO: 577, and the LCDR3 comprises the amino acid sequence of SEQ ID NO: 578.

In some specific embodiments, in the antibody or antigen binding fragment provided herein, the HCDR1 comprises the amino acid sequence of SEQ ID NO: 579, the HCDR2 comprises the amino acid sequence of SEQ ID NO: 580, the HCDR3 comprises the amino acid sequence of SEQ ID NO: 581, the LCDR1 comprises the amino acid sequence of SEQ ID NO: 582, the LCDR2 comprises the amino acid sequence of SEQ ID NO: 583, and the LCDR3 comprises the amino acid sequence of SEQ ID NO: 584.

In some specific embodiments, in the antibody or antigen binding fragment provided herein, the HCDR1 comprises the amino acid sequence of SEQ ID NO: 585, the HCDR2 comprises the amino acid sequence of SEQ ID NO: 586, the HCDR3 comprises the amino acid sequence of SEQ ID NO: 587, the LCDR1 comprises the amino acid sequence of SEQ ID NO: 588, the LCDR2 comprises the amino acid sequence of SEQ ID NO: 589, and the LCDR3 comprises the amino acid sequence of SEQ ID NO: 590.

In some specific embodiments, in the antibody or antigen binding fragment provided herein, the HCDR1 comprises the amino acid sequence of SEQ ID NO: 591, the HCDR2 comprises the amino acid sequence of SEQ ID NO: 592, the HCDR3 comprises the amino acid sequence of SEQ ID NO: 593, the LCDR1 comprises the amino acid sequence of SEQ ID NO: 594, the LCDR2 comprises the amino acid sequence of SEQ ID NO: 595, and the LCDR3 comprises the amino acid sequence of SEQ ID NO: 596.

In some specific embodiments, in the antibody or antigen binding fragment provided herein, the HCDR1 comprises the amino acid sequence of SEQ ID NO: 597, the HCDR2 comprises the amino acid sequence of SEQ ID NO: 598, the HCDR3 comprises the amino acid sequence of SEQ ID NO: 599, the LCDR1 comprises the amino acid sequence of SEQ ID NO: 600, the LCDR2 comprises the amino acid sequence of SEQ ID NO: 601, and the LCDR3 comprises the amino acid sequence of SEQ ID NO: 602.

In some specific embodiments, in the antibody or antigen binding fragment provided herein, the HCDR1 comprises the amino acid sequence of SEQ ID NO: 603, the HCDR2 comprises the amino acid sequence of SEQ ID NO: 604, the HCDR3 comprises the amino acid sequence of SEQ ID NO: 605, the LCDR1 comprises the amino acid sequence of SEQ ID NO: 606, the LCDR2 comprises the amino acid sequence of SEQ ID NO: 607, and the LCDR3 comprises the amino acid sequence of SEQ ID NO: 608.

In some specific embodiments, in the antibody or antigen binding fragment provided herein, the HCDR1 comprises the amino acid sequence of SEQ ID NO: 609, the HCDR2 comprises the amino acid sequence of SEQ ID NO: 610, the HCDR3 comprises the amino acid sequence of SEQ ID NO: 611, the LCDR1 comprises the amino acid sequence of SEQ ID NO: 612, the LCDR2 comprises the amino acid sequence of SEQ ID NO: 613, and the LCDR3 comprises the amino acid sequence of SEQ ID NO: 614.

In some specific embodiments, in the antibody or antigen binding fragment provided herein, the HCDR1 comprises the amino acid sequence of SEQ ID NO: 615, the HCDR2 comprises the amino acid sequence of SEQ ID NO: 616, the HCDR3 comprises the amino acid sequence of SEQ ID NO: 617, the LCDR1 comprises the amino acid sequence of SEQ ID NO: 618, the LCDR2 comprises the amino acid sequence of SEQ ID NO: 619, and the LCDR3 comprises the amino acid sequence of SEQ ID NO: 620.

In some specific embodiments, in the antibody or antigen binding fragment provided herein, the HCDR1 comprises the amino acid sequence of SEQ ID NO: 621, the HCDR2 comprises the amino acid sequence of SEQ ID NO: 622, the HCDR3 comprises the amino acid sequence of SEQ ID NO: 623, the LCDR1 comprises the amino acid sequence of SEQ ID NO: 624, the LCDR2 comprises the amino acid sequence of SEQ ID NO: 625, and the LCDR3 comprises the amino acid sequence of SEQ ID NO: 626.

In some specific embodiments, in the antibody or antigen binding fragment provided herein, the HCDR1 comprises the amino acid sequence of SEQ ID NO: 627, the HCDR2 comprises the amino acid sequence of SEQ ID NO: 628, the HCDR3 comprises the amino acid sequence of SEQ ID NO: 629, the LCDR1 comprises the amino acid sequence of SEQ ID NO: 630, the LCDR2 comprises the amino acid sequence of SEQ ID NO: 631, and the LCDR3 comprises the amino acid sequence of SEQ ID NO: 632.

In some specific embodiments, in the antibody or antigen binding fragment provided herein, the HCDR1 comprises the amino acid sequence of SEQ ID NO: 633, the HCDR2 comprises the amino acid sequence of SEQ ID NO: 634, the HCDR3 comprises the amino acid sequence of SEQ ID NO: 635, the LCDR1 comprises the amino acid sequence of SEQ ID NO: 636, the LCDR2 comprises the amino acid sequence of SEQ ID NO: 637, and the LCDR3 comprises the amino acid sequence of SEQ ID NO: 638.

In some specific embodiments, in the antibody or antigen binding fragment provided herein, the HCDR1 comprises the amino acid sequence of SEQ ID NO: 639, the HCDR2 comprises the amino acid sequence of SEQ ID NO: 640, the HCDR3 comprises the amino acid sequence of SEQ ID NO: 641, the LCDR1 comprises the amino acid sequence of SEQ ID NO: 642, the LCDR2 comprises the amino acid sequence of SEQ ID NO: 643, and the LCDR3 comprises the amino acid sequence of SEQ ID NO: 644.

In some specific embodiments, in the antibody or antigen binding fragment provided herein, the HCDR1 comprises the amino acid sequence of SEQ ID NO: 645, the HCDR2 comprises the amino acid sequence of SEQ ID NO: 646, the HCDR3 comprises the amino acid sequence of SEQ ID NO: 647, the LCDR1 comprises the amino acid sequence of SEQ ID NO: 648, the LCDR2 comprises the amino acid sequence of SEQ ID NO: 649, and the LCDR3 comprises the amino acid sequence of SEQ ID NO: 650.

In some specific embodiments, in the antibody or antigen binding fragment provided herein, the HCDR1 comprises the amino acid sequence of SEQ ID NO: 651, the HCDR2 comprises the amino acid sequence of SEQ ID NO: 652, the HCDR3 comprises the amino acid sequence of SEQ ID NO: 653, the LCDR1 comprises the amino acid sequence of SEQ ID NO: 654, the LCDR2 comprises the amino acid sequence of SEQ ID NO: 655, and the LCDR3 comprises the amino acid sequence of SEQ ID NO: 656.

In some specific embodiments, in the antibody or antigen binding fragment provided herein, the HCDR1 comprises the amino acid sequence of SEQ ID NO: 657, the HCDR2 comprises the amino acid sequence of SEQ ID NO: 658, the HCDR3 comprises the amino acid sequence of SEQ ID NO: 659, the LCDR1 comprises the amino acid sequence of SEQ ID NO: 660, the LCDR2 comprises the amino acid sequence of SEQ ID NO: 661, and the LCDR3 comprises the amino acid sequence of SEQ ID NO: 662.

In some specific embodiments, in the antibody or antigen binding fragment provided herein, the HCDR1 comprises the amino acid sequence of SEQ ID NO: 663, the HCDR2 comprises the amino acid sequence of SEQ ID NO: 664, the HCDR3 comprises the amino acid sequence of SEQ ID NO: 665, the LCDR1 comprises the amino acid sequence of SEQ ID NO: 666, the LCDR2 comprises the amino acid sequence of SEQ ID NO: 667, and the LCDR3 comprises the amino acid sequence of SEQ ID NO: 668.

In some specific embodiments, in the antibody or antigen binding fragment provided herein, the HCDR1 comprises the amino acid sequence of SEQ ID NO: 669, the HCDR2 comprises the amino acid sequence of SEQ ID NO: 670, the HCDR3 comprises the amino acid sequence of SEQ ID NO: 671, the LCDR1 comprises the amino acid sequence of SEQ ID NO: 672, the LCDR2 comprises the amino acid sequence of SEQ ID NO: 673, and the LCDR3 comprises the amino acid sequence of SEQ ID NO: 674.

In some specific embodiments, in the antibody or antigen binding fragment provided herein, the HCDR1 comprises the amino acid sequence of SEQ ID NO: 675, the HCDR2 comprises the amino acid sequence of SEQ ID NO: 676, the HCDR3 comprises the amino acid sequence of SEQ ID NO: 677, the LCDR1 comprises the amino acid sequence of SEQ ID NO: 678, the LCDR2 comprises the amino acid sequence of SEQ ID NO: 679, and the LCDR3 comprises the amino acid sequence of SEQ ID NO: 680.

In some specific embodiments, in the antibody or antigen binding fragment provided herein, the HCDR1 comprises the amino acid sequence of SEQ ID NO: 681, the HCDR2 comprises the amino acid sequence of SEQ ID NO: 682, the HCDR3 comprises the amino acid sequence of SEQ ID NO: 683, the LCDR1 comprises the amino acid sequence of SEQ ID NO: 684, the LCDR2 comprises the amino acid sequence of SEQ ID NO: 685, and the LCDR3 comprises the amino acid sequence of SEQ ID NO: 686.

In some specific embodiments, in the antibody or antigen binding fragment provided herein, the HCDR1 comprises the amino acid sequence of SEQ ID NO: 687, the HCDR2 comprises the amino acid sequence of SEQ ID NO: 688, the HCDR3 comprises the amino acid sequence of SEQ ID NO: 689, the LCDR1 comprises the amino acid sequence of SEQ ID NO: 690, the LCDR2 comprises the amino acid sequence of SEQ ID NO: 691, and the LCDR3 comprises the amino acid sequence of SEQ ID NO: 692.

In some specific embodiments, in the antibody or antigen binding fragment provided herein, the HCDR1 comprises the amino acid sequence of SEQ ID NO: 693, the HCDR2 comprises the amino acid sequence of SEQ ID NO: 694, the HCDR3 comprises the amino acid sequence of SEQ ID NO: 695, the LCDR1 comprises the amino acid sequence of SEQ ID NO: 696, the LCDR2 comprises the amino acid sequence of SEQ ID NO: 697, and the LCDR3 comprises the amino acid sequence of SEQ ID NO: 698.

In some specific embodiments, in the antibody or antigen binding fragment provided herein, the HCDR1 comprises the amino acid sequence of SEQ ID NO: 699, the HCDR2 comprises the amino acid sequence of SEQ ID NO: 700, the HCDR3 comprises the amino acid sequence of SEQ ID NO: 701, the LCDR1 comprises the amino acid sequence of SEQ ID NO: 702, the LCDR2 comprises the amino acid sequence of SEQ ID NO: 703, and the LCDR3 comprises the amino acid sequence of SEQ ID NO: 704.

In some specific embodiments, in the antibody or antigen binding fragment provided herein, the HCDR1 comprises the amino acid sequence of SEQ ID NO: 705, the HCDR2 comprises the amino acid sequence of SEQ ID NO: 706, the HCDR3 comprises the amino acid sequence of SEQ ID NO: 707, the LCDR1 comprises the amino acid sequence of SEQ ID NO: 708, the LCDR2 comprises the amino acid sequence of SEQ ID NO: 709, and the LCDR3 comprises the amino acid sequence of SEQ ID NO: 710.

In some specific embodiments, in the antibody or antigen binding fragment provided herein, the HCDR1 comprises the amino acid sequence of SEQ ID NO: 711, the HCDR2 comprises the amino acid sequence of SEQ ID NO: 712, the HCDR3 comprises the amino acid sequence of SEQ ID NO: 713, the LCDR1 comprises the amino acid sequence of SEQ ID NO: 714, the LCDR2 comprises the amino acid sequence of SEQ ID NO: 715, and the LCDR3 comprises the amino acid sequence of SEQ ID NO: 716.

In some specific embodiments, in the antibody or antigen binding fragment provided herein, the HCDR1 comprises the amino acid sequence of SEQ ID NO: 717, the HCDR2 comprises the amino acid sequence of SEQ ID NO: 718, the HCDR3 comprises the amino acid sequence of SEQ ID NO: 719, the LCDR1 comprises the amino acid sequence of SEQ ID NO: 720, the LCDR2 comprises the amino acid sequence of SEQ ID NO: 721, and the LCDR3 comprises the amino acid sequence of SEQ ID NO: 722.

In some specific embodiments, in the antibody or antigen binding fragment provided herein, the HCDR1 comprises the amino acid sequence of SEQ ID NO: 723, the HCDR2 comprises the amino acid sequence of SEQ ID NO: 724, the HCDR3 comprises the amino acid sequence of SEQ ID NO: 725, the LCDR1 comprises the amino acid sequence of SEQ ID NO: 726, the LCDR2 comprises the amino acid sequence of SEQ ID NO: 727, and the LCDR3 comprises the amino acid sequence of SEQ ID NO: 728.

In some specific embodiments, in the antibody or antigen binding fragment provided herein, the HCDR1 comprises the amino acid sequence of SEQ ID NO: 729, the HCDR2 comprises the amino acid sequence of SEQ ID NO: 730, the HCDR3 comprises the amino acid sequence of SEQ ID NO: 731, the LCDR1 comprises the amino acid sequence of SEQ ID NO: 732, the LCDR2 comprises the amino acid sequence of SEQ ID NO: 733, and the LCDR3 comprises the amino acid sequence of SEQ ID NO: 734.

In some specific embodiments, in the antibody or antigen binding fragment provided herein, the HCDR1 comprises the amino acid sequence of SEQ ID NO: 735, the HCDR2 comprises the amino acid sequence of SEQ ID NO: 736, the HCDR3 comprises the amino acid sequence of SEQ ID NO: 737, the LCDR1 comprises the amino acid sequence of SEQ ID NO: 738, the LCDR2 comprises the amino acid sequence of SEQ ID NO: 739, and the LCDR3 comprises the amino acid sequence of SEQ ID NO: 740.

In some specific embodiments, in the antibody or antigen binding fragment provided herein, the HCDR1 comprises the amino acid sequence of SEQ ID NO: 741, the HCDR2 comprises the amino acid sequence of SEQ ID NO: 742, the HCDR3 comprises the amino acid sequence of SEQ ID NO: 743, the LCDR1 comprises the amino acid sequence of SEQ ID NO: 744, the LCDR2 comprises the amino acid sequence of SEQ ID NO: 745, and the LCDR3 comprises the amino acid sequence of SEQ ID NO: 746.

In some specific embodiments, in the antibody or antigen binding fragment provided herein, the HCDR1 comprises the amino acid sequence of SEQ ID NO: 747, the HCDR2 comprises the amino acid sequence of SEQ ID NO: 748, the HCDR3 comprises the amino acid sequence of SEQ ID NO: 749, the LCDR1 comprises the amino acid sequence of SEQ ID NO: 750, the LCDR2 comprises the amino acid sequence of SEQ ID NO: 751, and the LCDR3 comprises the amino acid sequence of SEQ ID NO: 752.

In some specific embodiments, in the antibody or antigen binding fragment provided herein, the HCDR1 comprises the amino acid sequence of SEQ ID NO: 753, the HCDR2 comprises the amino acid sequence of SEQ ID NO: 754, the HCDR3 comprises the amino acid sequence of SEQ ID NO: 755, the LCDR1 comprises the amino acid sequence of SEQ ID NO: 756, the LCDR2 comprises the amino acid sequence of SEQ ID NO: 757, and the LCDR3 comprises the amino acid sequence of SEQ ID NO: 758.

In some specific embodiments, in the antibody or antigen binding fragment provided herein, the HCDR1 comprises the amino acid sequence of SEQ ID NO: 759, the HCDR2 comprises the amino acid sequence of SEQ ID NO: 760, the HCDR3 comprises the amino acid sequence of SEQ ID NO: 761, the LCDR1 comprises the amino acid sequence of SEQ ID NO: 762, the LCDR2 comprises the amino acid sequence of SEQ ID NO: 763, and the LCDR3 comprises the amino acid sequence of SEQ ID NO: 764.

In some specific embodiments, in the antibody or antigen binding fragment provided herein, the HCDR1 comprises the amino acid sequence of SEQ ID NO: 765, the HCDR2 comprises the amino acid sequence of SEQ ID NO: 766, the HCDR3 comprises the amino acid sequence of SEQ ID NO: 767, the LCDR1 comprises the amino acid sequence of SEQ ID NO: 768, the LCDR2 comprises the amino acid sequence of SEQ ID NO: 769, and the LCDR3 comprises the amino acid sequence of SEQ ID NO: 770.

In some specific embodiments, in the antibody or antigen binding fragment provided herein, the HCDR1 comprises the amino acid sequence of SEQ ID NO: 771, the HCDR2 comprises the amino acid sequence of SEQ ID NO: 772, the HCDR3 comprises the amino acid sequence of SEQ ID NO: 773, the LCDR1 comprises the amino acid sequence of SEQ ID NO: 774, the LCDR2 comprises the amino acid sequence of SEQ ID NO: 775, and the LCDR3 comprises the amino acid sequence of SEQ ID NO: 776.

In some specific embodiments, in the antibody or antigen binding fragment provided herein, the HCDR1 comprises the amino acid sequence of SEQ ID NO: 777, the HCDR2 comprises the amino acid sequence of SEQ ID NO: 778, the HCDR3 comprises the amino acid sequence of SEQ ID NO: 779, the LCDR1 comprises the amino acid sequence of SEQ ID NO: 780, the LCDR2 comprises the amino acid sequence of SEQ ID NO: 781, and the LCDR3 comprises the amino acid sequence of SEQ ID NO: 782.

In some specific embodiments, in the antibody or antigen binding fragment provided herein, the HCDR1 comprises the amino acid sequence of SEQ ID NO: 783, the HCDR2 comprises the amino acid sequence of SEQ ID NO: 784, the HCDR3 comprises the amino acid sequence of SEQ ID NO: 785, the LCDR1 comprises the amino acid sequence of SEQ ID NO: 786, the LCDR2 comprises the amino acid sequence of SEQ ID NO: 787, and the LCDR3 comprises the amino acid sequence of SEQ ID NO: 788.

In some specific embodiments, in the antibody or antigen binding fragment provided herein, the HCDR1 comprises the amino acid sequence of SEQ ID NO: 789, the HCDR2 comprises the amino acid sequence of SEQ ID NO: 790, the HCDR3 comprises the amino acid sequence of SEQ ID NO: 791, the LCDR1 comprises the amino acid sequence of SEQ ID NO: 792, the LCDR2 comprises the amino acid sequence of SEQ ID NO: 793, and the LCDR3 comprises the amino acid sequence of SEQ ID NO: 794.

In some specific embodiments, in the antibody or antigen binding fragment provided herein, the HCDR1 comprises the amino acid sequence of SEQ ID NO: 795, the HCDR2 comprises the amino acid sequence of SEQ ID NO: 796, the HCDR3 comprises the amino acid sequence of SEQ ID NO: 797, the LCDR1 comprises the amino acid sequence of SEQ ID NO: 798, the LCDR2 comprises the amino acid sequence of SEQ ID NO: 799, and the LCDR3 comprises the amino acid sequence of SEQ ID NO: 800.

In some specific embodiments, in the antibody or antigen binding fragment provided herein, the HCDR1 comprises the amino acid sequence of SEQ ID NO: 801, the HCDR2 comprises the amino acid sequence of SEQ ID NO: 802, the HCDR3 comprises the amino acid sequence of SEQ ID NO: 803, the LCDR1 comprises the amino acid sequence of SEQ ID NO: 804, the LCDR2 comprises the amino acid sequence of SEQ ID NO: 805, and the LCDR3 comprises the amino acid sequence of SEQ ID NO: 806.

In some specific embodiments, in the antibody or antigen binding fragment provided herein, the HCDR1 comprises the amino acid sequence of SEQ ID NO: 807, the HCDR2 comprises the amino acid sequence of SEQ ID NO: 808, the HCDR3 comprises the amino acid sequence of SEQ ID NO: 809, the LCDR1 comprises the amino acid sequence of SEQ ID NO: 810, the LCDR2 comprises the amino acid sequence of SEQ ID NO: 811, and the LCDR3 comprises the amino acid sequence of SEQ ID NO: 812.

In some specific embodiments, in the antibody or antigen binding fragment provided herein, the HCDR1 comprises the amino acid sequence of SEQ ID NO: 813, the HCDR2 comprises the amino acid sequence of SEQ ID NO: 814, the HCDR3 comprises the amino acid sequence of SEQ ID NO: 815, the LCDR1 comprises the amino acid sequence of SEQ ID NO: 816, the LCDR2 comprises the amino acid sequence of SEQ ID NO: 817, and the LCDR3 comprises the amino acid sequence of SEQ ID NO: 818.

In some specific embodiments, in the antibody or antigen binding fragment provided herein, the HCDR1 comprises the amino acid sequence of SEQ ID NO: 819, the HCDR2 comprises the amino acid sequence of SEQ ID NO: 820, the HCDR3 comprises the amino acid sequence of SEQ ID NO: 821, the LCDR1 comprises the amino acid sequence of SEQ ID NO: 822, the LCDR2 comprises the amino acid sequence of SEQ ID NO: 823, and the LCDR3 comprises the amino acid sequence of SEQ ID NO: 824.

In some specific embodiments, in the antibody or antigen binding fragment provided herein, the HCDR1 comprises the amino acid sequence of SEQ ID NO: 825, the HCDR2 comprises the amino acid sequence of SEQ ID NO: 826, the HCDR3 comprises the amino acid sequence of SEQ ID NO: 827, the LCDR1 comprises the amino acid sequence of SEQ ID NO: 828, the LCDR2 comprises the amino acid sequence of SEQ ID NO: 829, and the LCDR3 comprises the amino acid sequence of SEQ ID NO: 830.

In some specific embodiments, in the antibody or antigen binding fragment provided herein, the HCDR1 comprises the amino acid sequence of SEQ ID NO: 831, the HCDR2 comprises the amino acid sequence of SEQ ID NO: 832, the HCDR3 comprises the amino acid sequence of SEQ ID NO: 833, the LCDR1 comprises the amino acid sequence of SEQ ID NO: 834, the LCDR2 comprises the amino acid sequence of SEQ ID NO: 835, and the LCDR3 comprises the amino acid sequence of SEQ ID NO: 836.

In some specific embodiments, in the antibody or antigen binding fragment provided herein, the HCDR1 comprises the amino acid sequence of SEQ ID NO: 837, the HCDR2 comprises the amino acid sequence of SEQ ID NO: 838, the HCDR3 comprises the amino acid sequence of SEQ ID NO: 839, the LCDR1 comprises the amino acid sequence of SEQ ID NO: 840, the LCDR2 comprises the amino acid sequence of SEQ ID NO: 841, and the LCDR3 comprises the amino acid sequence of SEQ ID NO: 842.

In some specific embodiments, in the antibody or antigen binding fragment provided herein, the HCDR1 comprises the amino acid sequence of SEQ ID NO: 843, the HCDR2 comprises the amino acid sequence of SEQ ID NO: 844, the HCDR3 comprises the amino acid sequence of SEQ ID NO: 845, the LCDR1 comprises the amino acid sequence of SEQ ID NO: 846, the LCDR2 comprises the amino acid sequence of SEQ ID NO: 847, and the LCDR3 comprises the amino acid sequence of SEQ ID NO: 848.

In some specific embodiments, in the antibody or antigen binding fragment provided herein, the HCDR1 comprises the amino acid sequence of SEQ ID NO: 543, the HCDR2 comprises the amino acid sequence of SEQ ID NO: 544, the HCDR3 comprises the amino acid sequence of SEQ ID NO: 1179, the LCDR1 comprises the amino acid sequence of SEQ ID NO: 546, the LCDR2 comprises the amino acid sequence of SEQ ID NO: 1281, and the LCDR3 comprises the amino acid sequence of SEQ ID NO: 548.

In some embodiments, the antibody further comprises one or more framework regions of SEQ ID NOs: 283-400, and 1155-1166.

In some embodiments, the antibody provided herein is a humanized antibody. Framework regions described herein are determined based upon the boundaries of the CDR numbering system. In other words, if the CDRs are determined by, e.g., Kabat, IMGT, or Chothia, then the framework regions are the amino acid residues surrounding the CDRs in the variable region in the format, from the N-terminus to C-terminus: FR1-CDR1-FR2-CDR2-FR3-CDR3-FR4. For example, FR1 is defined as the amino acid residues N-terminal to the CDR1 amino acid residues as defined by, e.g., the Kabat numbering system, the IMGT numbering system, or the Chothia numbering system, FR2 is defined as the amino acid residues between CDR1 and CDR2 amino acid residues as defined by, e.g., the Kabat numbering system, the IMGT numbering system, or the Chothia numbering system, FR3 is defined as the amino acid residues between CDR2 and CDR3 amino acid residues as defined by, e.g., the Kabat numbering system, the IMGT numbering system, or the Chothia numbering system, and FR4 is defined as the amino acid residues C-terminal to the CDR3 amino acid residues as defined by, e.g., the Kabat numbering system, the IMGT numbering system, or the Chothia numbering system.

In some embodiments, the antibody or antigen binding fragment provided herein comprises a VH comprising the amino acid sequence of SEQ ID NO: 283. In some embodiments, the antibody or antigen binding fragment provided herein comprises a VL comprising the amino acid sequence of SEQ ID NO: 284. In some embodiments, the antibody or antigen binding fragment provided herein comprises a VH comprising the amino acid sequence of SEQ ID NO: 283, and a VL comprising the amino acid sequence of SEQ ID NO: 284.

In some embodiments, the antibody or antigen binding fragment provided herein comprises a VH comprising the amino acid sequence of SEQ ID NO: 285. In some embodiments, the antibody or antigen binding fragment provided herein comprises a VL comprising the amino acid sequence of SEQ ID NO: 286. In some embodiments, the antibody or antigen binding fragment provided herein comprises a VH comprising the amino acid sequence of SEQ ID NO: 285, and a VL comprising the amino acid sequence of SEQ ID NO: 286.

In some embodiments, the antibody or antigen binding fragment provided herein comprises a VH comprising the amino acid sequence of SEQ ID NO: 287. In some embodiments, the antibody or antigen binding fragment provided herein comprises a VL comprising the amino acid sequence of SEQ ID NO: 288. In some embodiments, the antibody or antigen binding fragment provided herein comprises a VH comprising the amino acid sequence of SEQ ID NO: 287, and a VL comprising the amino acid sequence of SEQ ID NO: 288.

In some embodiments, the antibody or antigen binding fragment provided herein comprises a VH comprising the amino acid sequence of SEQ ID NO: 289. In some embodiments, the antibody or antigen binding fragment provided herein comprises a VL comprising the amino acid sequence of SEQ ID NO: 290. In some embodiments, the antibody or antigen binding fragment provided herein comprises a VH comprising the amino acid sequence of SEQ ID NO: 289, and a VL comprising the amino acid sequence of SEQ ID NO: 290.

In some embodiments, the antibody or antigen binding fragment provided herein comprises a VH comprising the amino acid sequence of SEQ ID NO: 291. In some embodiments, the antibody or antigen binding fragment provided herein comprises a VL comprising the amino acid sequence of SEQ ID NO: 292. In some embodiments, the antibody or antigen binding fragment provided herein comprises a VH comprising the amino acid sequence of SEQ ID NO: 291, and a VL comprising the amino acid sequence of SEQ ID NO: 292.

In some embodiments, the antibody or antigen binding fragment provided herein comprises a VH comprising the amino acid sequence of SEQ ID NO: 293. In some embodiments, the antibody or antigen binding fragment provided herein comprises a VL comprising the amino acid sequence of SEQ ID NO: 294. In some embodiments, the antibody or antigen binding fragment provided herein comprises a VH comprising the amino acid sequence of SEQ ID NO: 293, and a VL comprising the amino acid sequence of SEQ ID NO: 294.

In some embodiments, the antibody or antigen binding fragment provided herein comprises a VH comprising the amino acid sequence of SEQ ID NO: 295. In some embodiments, the antibody or antigen binding fragment provided herein comprises a VL comprising the amino acid sequence of SEQ ID NO: 296. In some embodiments, the antibody or antigen binding fragment provided herein comprises a VH comprising the amino acid sequence of SEQ ID NO: 295, and a VL comprising the amino acid sequence of SEQ ID NO: 296.

In some embodiments, the antibody or antigen binding fragment provided herein comprises a VH comprising the amino acid sequence of SEQ ID NO: 297. In some embodiments, the antibody or antigen binding fragment provided herein comprises a VL comprising the amino acid sequence of SEQ ID NO: 298. In some embodiments, the antibody or antigen binding fragment provided herein comprises a VH comprising the amino acid sequence of SEQ ID NO: 297, and a VL comprising the amino acid sequence of SEQ ID NO: 298.

In some embodiments, the antibody or antigen binding fragment provided herein comprises a VH comprising the amino acid sequence of SEQ ID NO: 299. In some embodiments, the antibody or antigen binding fragment provided herein a VL comprising the amino acid sequence of SEQ ID NO: 300. In some embodiments, the antibody or antigen binding fragment provided herein comprises a VH comprising the amino acid sequence of SEQ ID NO: 299, and a VL comprising the amino acid sequence of SEQ ID NO: 300.

In some embodiments, the antibody or antigen binding fragment provided herein comprises a VH comprising the amino acid sequence of SEQ ID NO: 301. In some embodiments, the antibody or antigen binding fragment provided herein comprises a VL comprising the amino acid sequence of SEQ ID NO: 302. In some embodiments, the antibody or antigen binding fragment provided herein comprises a VH comprising the amino acid sequence of SEQ ID NO: 301, and a VL comprising the amino acid sequence of SEQ ID NO: 302.

In some embodiments, the antibody or antigen binding fragment provided herein comprises a VH comprising the amino acid sequence of SEQ ID NO: 303. In some embodiments, the antibody or antigen binding fragment provided herein comprises a VL comprising the amino acid sequence of SEQ ID NO: 304. In some embodiments, the antibody or antigen binding fragment provided herein comprises a VH comprising the amino acid sequence of SEQ ID NO: 303, and a VL comprising the amino acid sequence of SEQ ID NO: 304.

In some embodiments, the antibody or antigen binding fragment provided herein comprises a VH comprising the amino acid sequence of SEQ ID NO: 305. In some embodiments, the antibody or antigen binding fragment provided herein comprises a VL comprising the amino acid sequence of SEQ ID NO: 306. In some embodiments, the antibody or antigen binding fragment provided herein comprises a VH comprising the amino acid sequence of SEQ ID NO: 305, and a VL comprising the amino acid sequence of SEQ ID NO: 306.

In some embodiments, the antibody or antigen binding fragment provided herein comprises a VH comprising the amino acid sequence of SEQ ID NO: 307. In some embodiments, the antibody or antigen binding fragment provided herein comprises a VL comprising the amino acid sequence of SEQ ID NO: 308. In some embodiments, the antibody or antigen binding fragment provided herein comprises a VH comprising the amino acid sequence of SEQ ID NO: 307, and a VL comprising the amino acid sequence of SEQ ID NO: 308.

In some embodiments, the antibody or antigen binding fragment provided herein comprises a VH comprising the amino acid sequence of SEQ ID NO: 309. In some embodiments, the antibody or antigen binding fragment provided herein comprises a VL comprising the amino acid sequence of SEQ ID NO: 310. In some embodiments, the antibody or antigen binding fragment provided herein comprises a VH comprising the amino acid sequence of SEQ ID NO: 309, and a VL comprising the amino acid sequence of SEQ ID NO: 310.

In some embodiments, the antibody or antigen binding fragment provided herein comprises a VH comprising the amino acid sequence of SEQ ID NO: 311. In some embodiments, the antibody or antigen binding fragment provided herein comprises a VL comprising the amino acid sequence of SEQ ID NO: 312. In some embodiments, the antibody or antigen binding fragment provided herein comprises a VH comprising the amino acid sequence of SEQ ID NO: 311, and a VL comprising the amino acid sequence of SEQ ID NO: 312.

In some embodiments, the antibody or antigen binding fragment provided herein comprises a VH comprising the amino acid sequence of SEQ ID NO: 313. In some embodiments, the antibody or antigen binding fragment provided herein comprises a VL comprising the amino acid sequence of SEQ ID NO: 314. In some embodiments, the antibody or antigen binding fragment provided herein comprises a VH comprising the amino acid sequence of SEQ ID NO: 313, and a VL comprising the amino acid sequence of SEQ ID NO: 314.

In some embodiments, the antibody or antigen binding fragment provided herein comprises a VH comprising the amino acid sequence of SEQ ID NO: 315. In some embodiments, the antibody or antigen binding fragment provided herein comprises a VL comprising the amino acid sequence of SEQ ID NO: 316. In some embodiments, the antibody or antigen binding fragment provided herein comprises a VH comprising the amino acid sequence of SEQ ID NO: 315, and a VL comprising the amino acid sequence of SEQ ID NO: 316.

In some embodiments, the antibody or antigen binding fragment provided herein comprises a VH comprising the amino acid sequence of SEQ ID NO: 317. In some embodiments, the antibody or antigen binding fragment provided herein comprises a VL comprising the amino acid sequence of SEQ ID NO: 318. In some embodiments, the antibody or antigen binding fragment provided herein comprises a VH comprising the amino acid sequence of SEQ ID NO: 317, and a VL comprising the amino acid sequence of SEQ ID NO: 318.

In some embodiments, the antibody or antigen binding fragment provided herein comprises a VH comprising the amino acid sequence of SEQ ID NO: 319. In some embodiments, the antibody or antigen binding fragment provided herein comprises a VL comprising the amino acid sequence of SEQ ID NO: 320. In some embodiments, the antibody or antigen binding fragment provided herein comprises a VH comprising the amino acid sequence of SEQ ID NO: 319, and a VL comprising the amino acid sequence of SEQ ID NO: 320.

In some embodiments, the antibody or antigen binding fragment provided herein comprises a VH comprising the amino acid sequence of SEQ ID NO: 321. In some embodiments, the antibody or antigen binding fragment provided herein comprises a VL comprising the amino acid sequence of SEQ ID NO: 322. In some embodiments, the antibody or antigen binding fragment provided herein comprises a VH comprising the amino acid sequence of SEQ ID NO: 321, and a VL comprising the amino acid sequence of SEQ ID NO: 322.

In some embodiments, the antibody or antigen binding fragment provided herein comprises a VH comprising the amino acid sequence of SEQ ID NO: 323. In some embodiments, the antibody or antigen binding fragment provided herein comprises a VL comprising the amino acid sequence of SEQ ID NO: 324. In some embodiments, the antibody or antigen binding fragment provided herein comprises a VH comprising the amino acid sequence of SEQ ID NO: 323, and a VL comprising the amino acid sequence of SEQ ID NO: 324.

In some embodiments, the antibody or antigen binding fragment provided herein comprises a VH comprising the amino acid sequence of SEQ ID NO: 325. In some embodiments, the antibody or antigen binding fragment provided herein comprises a VL comprising the amino acid sequence of SEQ ID NO: 326. In some embodiments, the antibody or antigen binding fragment provided herein comprises a VH comprising the amino acid sequence of SEQ ID NO: 325, and a VL comprising the amino acid sequence of SEQ ID NO: 326.

In some embodiments, the antibody or antigen binding fragment provided herein comprises a VH comprising the amino acid sequence of SEQ ID NO: 327. In some embodiments, the antibody or antigen binding fragment provided herein comprises a VL comprising the amino acid sequence of SEQ ID NO: 328. In some embodiments, the antibody or antigen binding fragment provided herein comprises a VH comprising the amino acid sequence of SEQ ID NO: 327, and a VL comprising the amino acid sequence of SEQ ID NO: 328.

In some embodiments, the antibody or antigen binding fragment provided herein comprises a VH comprising the amino acid sequence of SEQ ID NO: 329. In some embodiments, the antibody or antigen binding fragment provided herein comprises a VL comprising the amino acid sequence of SEQ ID NO: 330. In some embodiments, the antibody or antigen binding fragment provided herein comprises a VH comprising the amino acid sequence of SEQ ID NO: 329, and a VL comprising the amino acid sequence of SEQ ID NO: 330.

In some embodiments, the antibody or antigen binding fragment provided herein comprises a VH comprising the amino acid sequence of SEQ ID NO: 331. In some embodiments, the antibody or antigen binding fragment provided herein comprises a VL comprising the amino acid sequence of SEQ ID NO: 332. In some embodiments, the antibody or antigen binding fragment provided herein comprises a VH comprising the amino acid sequence of SEQ ID NO: 331, and a VL comprising the amino acid sequence of SEQ ID NO: 332.

In some embodiments, the antibody or antigen binding fragment provided herein comprises a VH comprising the amino acid sequence of SEQ ID NO: 333. In some embodiments, the antibody or antigen binding fragment provided herein comprises a VL comprising the amino acid sequence of SEQ ID NO: 334. In some embodiments, the antibody or antigen binding fragment provided herein comprises a VH comprising the amino acid sequence of SEQ ID NO: 333, and a VL comprising the amino acid sequence of SEQ ID NO: 334.

In some embodiments, the antibody or antigen binding fragment provided herein comprises a VH comprising the amino acid sequence of SEQ ID NO: 335. In some embodiments, the antibody or antigen binding fragment provided herein comprises a VL comprising the amino acid sequence of SEQ ID NO: 336. In some embodiments, the antibody or antigen binding fragment provided herein comprises a VH comprising the amino acid sequence of SEQ ID NO: 335, and a VL comprising the amino acid sequence of SEQ ID NO: 336.

In some embodiments, the antibody or antigen binding fragment provided herein comprises a VH comprising the amino acid sequence of SEQ ID NO: 337. In some embodiments, the antibody or antigen binding fragment provided herein comprises a VL comprising the amino acid sequence of SEQ ID NO: 338. In some embodiments, the antibody or antigen binding fragment provided herein comprises a VH comprising the amino acid sequence of SEQ ID NO: 337, and a VL comprising the amino acid sequence of SEQ ID NO: 338.

In some embodiments, the antibody or antigen binding fragment provided herein comprises a VH comprising the amino acid sequence of SEQ ID NO: 339. In some embodiments, the antibody or antigen binding fragment provided herein comprises a VL comprising the amino acid sequence of SEQ ID NO: 340. In some embodiments, the antibody or antigen binding fragment provided herein comprises a VH comprising the amino acid sequence of SEQ ID NO: 339, and a VL comprising the amino acid sequence of SEQ ID NO: 340.

In some embodiments, the antibody or antigen binding fragment provided herein comprises a VH comprising the amino acid sequence of SEQ ID NO: 341. In some embodiments, the antibody or antigen binding fragment provided herein comprises a VL comprising the amino acid sequence of SEQ ID NO: 342. In some embodiments, the antibody or antigen binding fragment provided herein comprises a VH comprising the amino acid sequence of SEQ ID NO: 341, and a VL comprising the amino acid sequence of SEQ ID NO: 342.

In some embodiments, the antibody or antigen binding fragment provided herein comprises a VH comprising the amino acid sequence of SEQ ID NO: 343. In some embodiments, the antibody or antigen binding fragment provided herein comprises a VL comprising the amino acid sequence of SEQ ID NO: 344. In some embodiments, the antibody or antigen binding fragment provided herein comprises a VH comprising the amino acid sequence of SEQ ID NO: 343, and a VL comprising the amino acid sequence of SEQ ID NO: 344.

In some embodiments, the antibody or antigen binding fragment provided herein comprises a VH comprising the amino acid sequence of SEQ ID NO: 345. In some embodiments, the antibody or antigen binding fragment provided herein comprises a VL comprising the amino acid sequence of SEQ ID NO: 346. In some embodiments, the antibody or antigen binding fragment provided herein comprises a VH comprising the amino acid sequence of SEQ ID NO: 345, and a VL comprising the amino acid sequence of SEQ ID NO: 346.

In some embodiments, the antibody or antigen binding fragment provided herein comprises a VH comprising the amino acid sequence of SEQ ID NO: 347. In some embodiments, the antibody or antigen binding fragment provided herein comprises a VL comprising the amino acid sequence of SEQ ID NO: 348. In some embodiments, the antibody or antigen binding fragment provided herein comprises a VH comprising the amino acid sequence of SEQ ID NO: 347, and a VL comprising the amino acid sequence of SEQ ID NO: 348.

In some embodiments, the antibody or antigen binding fragment provided herein comprises a VH comprising the amino acid sequence of SEQ ID NO: 349. In some embodiments, the antibody or antigen binding fragment provided herein comprises a VL comprising the amino acid sequence of SEQ ID NO: 350. In some embodiments, the antibody or antigen binding fragment provided herein comprises a VH comprising the amino acid sequence of SEQ ID NO: 349, and a VL comprising the amino acid sequence of SEQ ID NO: 350.

In some embodiments, the antibody or antigen binding fragment provided herein comprises a VH comprising the amino acid sequence of SEQ ID NO: 351. In some embodiments, the antibody or antigen binding fragment provided herein comprises a VL comprising the amino acid sequence of SEQ ID NO: 352. In some embodiments, the antibody or antigen binding fragment provided herein comprises a VH comprising the amino acid sequence of SEQ ID NO: 351, and a VL comprising the amino acid sequence of SEQ ID NO: 352.

In some embodiments, the antibody or antigen binding fragment provided herein comprises a VH comprising the amino acid sequence of SEQ ID NO: 353. In some embodiments, the antibody or antigen binding fragment provided herein comprises a VL comprising the amino acid sequence of SEQ ID NO: 354. In some embodiments, the antibody or antigen binding fragment provided herein comprises a VH comprising the amino acid sequence of SEQ ID NO: 353, and a VL comprising the amino acid sequence of SEQ ID NO: 354.

In some embodiments, the antibody or antigen binding fragment provided herein comprises a VH comprising the amino acid sequence of SEQ ID NO: 355. In some embodiments, the antibody or antigen binding fragment provided herein comprises a VL comprising the amino acid sequence of SEQ ID NO: 356. In some embodiments, the antibody or antigen binding fragment provided herein comprises a VH comprising the amino acid sequence of SEQ ID NO: 355, and a VL comprising the amino acid sequence of SEQ ID NO: 356.

In some embodiments, the antibody or antigen binding fragment provided herein comprises a VH comprising the amino acid sequence of SEQ ID NO: 357. In some embodiments, the antibody or antigen binding fragment provided herein comprises a VL comprising the amino acid sequence of SEQ ID NO: 358. In some embodiments, the antibody or antigen binding fragment provided herein comprises a VH comprising the amino acid sequence of SEQ ID NO: 357, and a VL comprising the amino acid sequence of SEQ ID NO: 358.

In some embodiments, the antibody or antigen binding fragment provided herein comprises a VH comprising the amino acid sequence of SEQ ID NO: 359. In some embodiments, the antibody or antigen binding fragment provided herein comprises a VL comprising the amino acid sequence of SEQ ID NO: 360. In some embodiments, the antibody or antigen binding fragment provided herein comprises a VH comprising the amino acid sequence of SEQ ID NO: 359, and a VL comprising the amino acid sequence of SEQ ID NO: 360.

In some embodiments, the antibody or antigen binding fragment provided herein comprises a VH comprising the amino acid sequence of SEQ ID NO: 361. In some embodiments, the antibody or antigen binding fragment provided herein comprises a VL comprising the amino acid sequence of SEQ ID NO: 362. In some embodiments, the antibody or antigen binding fragment provided herein comprises a VH comprising the amino acid sequence of SEQ ID NO: 361, and a VL comprising the amino acid sequence of SEQ ID NO: 362.

In some embodiments, the antibody or antigen binding fragment provided herein comprises a VH comprising the amino acid sequence of SEQ ID NO: 363. In some embodiments, the antibody or antigen binding fragment provided herein comprises a VL comprising the amino acid sequence of SEQ ID NO: 364. In some embodiments, the antibody or antigen binding fragment provided herein comprises a VH comprising the amino acid sequence of SEQ ID NO: 363, and a VL comprising the amino acid sequence of SEQ ID NO: 364.

In some embodiments, the antibody or antigen binding fragment provided herein comprises a VH comprising the amino acid sequence of SEQ ID NO: 365. In some embodiments, the antibody or antigen binding fragment provided herein comprises a VL comprising the amino acid sequence of SEQ ID NO: 366. In some embodiments, the antibody or antigen binding fragment provided herein comprises a VH comprising the amino acid sequence of SEQ ID NO: 365, and a VL comprising the amino acid sequence of SEQ ID NO: 366.

In some embodiments, the antibody or antigen binding fragment provided herein comprises a VH comprising the amino acid sequence of SEQ ID NO: 367. In some embodiments, the antibody or antigen binding fragment provided herein comprises a VL comprising the amino acid sequence of SEQ ID NO: 368. In some embodiments, the antibody or antigen binding fragment provided herein comprises a VH comprising the amino acid sequence of SEQ ID NO: 367, and a VL comprising the amino acid sequence of SEQ ID NO: 368.

In some embodiments, the antibody or antigen binding fragment provided herein comprises a VH comprising the amino acid sequence of SEQ ID NO: 369. In some embodiments, the antibody or antigen binding fragment provided herein comprises a VL comprising the amino acid sequence of SEQ ID NO: 370. In some embodiments, the antibody or antigen binding fragment provided herein comprises a VH comprising the amino acid sequence of SEQ ID NO: 369, and a VL comprising the amino acid sequence of SEQ ID NO: 370.

In some embodiments, the antibody or antigen binding fragment provided herein comprises a VH comprising the amino acid sequence of SEQ ID NO: 371. In some embodiments, the antibody or antigen binding fragment provided herein comprises a VL comprising the amino acid sequence of SEQ ID NO: 372. In some embodiments, the antibody or antigen binding fragment provided herein comprises a VH comprising the amino acid sequence of SEQ ID NO: 371, and a VL comprising the amino acid sequence of SEQ ID NO: 372.

In some embodiments, the antibody or antigen binding fragment provided herein comprises a VH comprising the amino acid sequence of SEQ ID NO: 373. In some embodiments, the antibody or antigen binding fragment provided herein comprises a VL comprising the amino acid sequence of SEQ ID NO: 374. In some embodiments, the antibody or antigen binding fragment provided herein comprises a VH comprising the amino acid sequence of SEQ ID NO: 373, and a VL comprising the amino acid sequence of SEQ ID NO: 374.

In some embodiments, the antibody or antigen binding fragment provided herein comprises a VH comprising the amino acid sequence of SEQ ID NO: 375. In some embodiments, the antibody or antigen binding fragment provided herein comprises a VL comprising the amino acid sequence of SEQ ID NO: 376. In some embodiments, the antibody or antigen binding fragment provided herein comprises a VH comprising the amino acid sequence of SEQ ID NO: 375, and a VL comprising the amino acid sequence of SEQ ID NO: 376.

In some embodiments, the antibody or antigen binding fragment provided herein comprises a VH comprising the amino acid sequence of SEQ ID NO: 377. In some embodiments, the antibody or antigen binding fragment provided herein comprises a VL comprising the amino acid sequence of SEQ ID NO: 378. In some embodiments, the antibody or antigen binding fragment provided herein comprises a VH comprising the amino acid sequence of SEQ ID NO: 377, and a VL comprising the amino acid sequence of SEQ ID NO: 378.

In some embodiments, the antibody or antigen binding fragment provided herein comprises a VH comprising the amino acid sequence of SEQ ID NO: 379. In some embodiments, the antibody or antigen binding fragment provided herein comprises a VL comprising the amino acid sequence of SEQ ID NO: 380. In some embodiments, the antibody or antigen binding fragment provided herein comprises a VH comprising the amino acid sequence of SEQ ID NO: 379, and a VL comprising the amino acid sequence of SEQ ID NO: 380.

In some embodiments, the antibody or antigen binding fragment provided herein comprises a VH comprising the amino acid sequence of SEQ ID NO: 381. In some embodiments, the antibody or antigen binding fragment provided herein comprises a VL comprising the amino acid sequence of SEQ ID NO: 382. In some embodiments, the antibody or antigen binding fragment provided herein comprises a VH comprising the amino acid sequence of SEQ ID NO: 381, and a VL comprising the amino acid sequence of SEQ ID NO: 382.

In some embodiments, the antibody or antigen binding fragment provided herein comprises a VH comprising the amino acid sequence of SEQ ID NO: 383. In some embodiments, the antibody or antigen binding fragment provided herein comprises a VL comprising the amino acid sequence of SEQ ID NO: 384. In some embodiments, the antibody or antigen binding fragment provided herein comprises a VH comprising the amino acid sequence of SEQ ID NO: 383, and a VL comprising the amino acid sequence of SEQ ID NO: 384.

In some embodiments, the antibody or antigen binding fragment provided herein comprises a VH comprising the amino acid sequence of SEQ ID NO: 385. In some embodiments, the antibody or antigen binding fragment provided herein comprises a VL comprising the amino acid sequence of SEQ ID NO: 386. In some embodiments, the antibody or antigen binding fragment provided herein comprises a VH comprising the amino acid sequence of SEQ ID NO: 385, and a VL comprising the amino acid sequence of SEQ ID NO: 386.

In some embodiments, the antibody or antigen binding fragment provided herein comprises a VH comprising the amino acid sequence of SEQ ID NO: 387. In some embodiments, the antibody or antigen binding fragment provided herein comprises a VL comprising the amino acid sequence of SEQ ID NO: 388. In some embodiments, the antibody or antigen binding fragment provided herein comprises a VH comprising the amino acid sequence of SEQ ID NO: 387, and a VL comprising the amino acid sequence of SEQ ID NO: 388.

In some embodiments, the antibody or antigen binding fragment provided herein comprises a VH comprising the amino acid sequence of SEQ ID NO: 389. In some embodiments, the antibody or antigen binding fragment provided herein comprises a VL comprising the amino acid sequence of SEQ ID NO: 390. In some embodiments, the antibody or antigen binding fragment provided herein comprises a VH comprising the amino acid sequence of SEQ ID NO: 389, and a VL comprising the amino acid sequence of SEQ ID NO: 390.

In some embodiments, the antibody or antigen binding fragment provided herein comprises a VH comprising the amino acid sequence of SEQ ID NO: 391. In some embodiments, the antibody or antigen binding fragment provided herein comprises a VL comprising the amino acid sequence of SEQ ID NO: 392. In some embodiments, the antibody or antigen binding fragment provided herein comprises a VH comprising the amino acid sequence of SEQ ID NO: 391, and a VL comprising the amino acid sequence of SEQ ID NO: 392.

In some embodiments, the antibody or antigen binding fragment provided herein comprises a VH comprising the amino acid sequence of SEQ ID NO: 393. In some embodiments, the antibody or antigen binding fragment provided herein comprises a VL comprising the amino acid sequence of SEQ ID NO: 394. In some embodiments, the antibody or antigen binding fragment provided herein comprises a VH comprising the amino acid sequence of SEQ ID NO: 393, and a VL comprising the amino acid sequence of SEQ ID NO: 394.

In some embodiments, the antibody or antigen binding fragment provided herein comprises a VH comprising the amino acid sequence of SEQ ID NO: 395. In some embodiments, the antibody or antigen binding fragment provided herein comprises a VL comprising the amino acid sequence of SEQ ID NO: 396. In some embodiments, the antibody or antigen binding fragment provided herein comprises a VH comprising the amino acid sequence of SEQ ID NO: 395, and a VL comprising the amino acid sequence of SEQ ID NO: 396.

In some embodiments, the antibody or antigen binding fragment provided herein comprises a VH comprising the amino acid sequence of SEQ ID NO: 397. In some embodiments, the antibody or antigen binding fragment provided herein comprises a VL comprising the amino acid sequence of SEQ ID NO: 398. In some embodiments, the antibody or antigen binding fragment provided herein comprises a VH comprising the amino acid sequence of SEQ ID NO: 397, and a VL comprising the amino acid sequence of SEQ ID NO: 398.

In some embodiments, the antibody or antigen binding fragment provided herein comprises a VH comprising the amino acid sequence of SEQ ID NO: 399. In some embodiments, the antibody or antigen binding fragment provided herein comprises a VL comprising the amino acid sequence of SEQ ID NO: 400. In some embodiments, the antibody or antigen binding fragment provided herein comprises a VH comprising the amino acid sequence of SEQ ID NO: 399, and a VL comprising the amino acid sequence of SEQ ID NO: 400.

In some embodiments, the antibody or antigen binding fragment provided herein comprises a VH comprising the amino acid sequence of SEQ ID NO: 1155. In some embodiments, the antibody or antigen binding fragment provided herein comprises a VL comprising the amino acid sequence of SEQ ID NO: 1156. In some embodiments, the antibody or antigen binding fragment provided herein comprises a VH comprising the amino acid sequence of SEQ ID NO: 1155, and a VL comprising the amino acid sequence of SEQ ID NO: 1156.

In some embodiments, the antibody or antigen binding fragment provided herein comprises a VH comprising the amino acid sequence of SEQ ID NO: 1157. In some embodiments, the antibody or antigen binding fragment provided herein comprises a VL comprising the amino acid sequence of SEQ ID NO: 1158. In some embodiments, the antibody or antigen binding fragment provided herein comprises a VH comprising the amino acid sequence of SEQ ID NO: 1157, and a VL comprising the amino acid sequence of SEQ ID NO: 1158.

In some embodiments, the antibody or antigen binding fragment provided herein comprises a VH comprising the amino acid sequence of SEQ ID NO: 1159. In some embodiments, the antibody or antigen binding fragment provided herein comprises a VL comprising the amino acid sequence of SEQ ID NO: 1160. In some embodiments, the antibody or antigen binding fragment provided herein comprises a VH comprising the amino acid sequence of SEQ ID NO: 1159, and a VL comprising the amino acid sequence of SEQ ID NO: 1160.

In some embodiments, the antibody or antigen binding fragment provided herein comprises a VH comprising the amino acid sequence of SEQ ID NO: 1161. In some embodiments, the antibody or antigen binding fragment provided herein comprises a VL comprising the amino acid sequence of SEQ ID NO: 1162. In some embodiments, the antibody or antigen binding fragment provided herein comprises a VH comprising the amino acid sequence of SEQ ID NO: 1161, and a VL comprising the amino acid sequence of SEQ ID NO: 1162.

In some embodiments, the antibody or antigen binding fragment provided herein comprises a VH comprising the amino acid sequence of SEQ ID NO: 1163. In some embodiments, the antibody or antigen binding fragment provided herein a VL comprising the amino acid sequence of SEQ ID NO: 1164. In some embodiments, the antibody or antigen binding fragment provided herein comprises a VH comprising the amino acid sequence of SEQ ID NO: 1163, and a VL comprising the amino acid sequence of SEQ ID NO: 1164.

In some embodiments, the antibody or antigen binding fragment provided herein comprises a VH comprising the amino acid sequence of SEQ ID NO: 1165. In some embodiments, the antibody or antigen binding fragment provided herein comprises a VL comprising the amino acid sequence of SEQ ID NO: 1166. In some embodiments, the antibody or antigen binding fragment provided herein comprises a VH comprising the amino acid sequence of SEQ ID NO: 1165, and a VL comprising the amino acid sequence of SEQ ID NO: 1166.

In certain embodiments, an antibody described herein or an antigen binding fragment thereof comprises amino acid sequences with certain percent identity relative to any antibody provided herein, for example, those described in Section 7 below.

In certain embodiments, the antibody described herein or an antigen binding fragment thereof comprises amino acid sequences with certain percent identity (such as at least about 80%, or at least about 81%, or at least about 82%, or at least about 83%, or at least about 84%, or at least about 85%, or at least about 86%, or at least about 87%, or at least about 88%, or at least about 89%, or as at least about 90%, or at least about 91%, or at least about 92%, or at least about 93%, or at least about 94%, or at least about 95%, or at least about 96%, or at least about 97%, or at least about 98%, or at least about 99%, or higher) relative to any antibody or fragment thereof provided herein, for example, a CDR, VH or VL in Tables 5-6, and 14-15. In some embodiments, the antibody described herein or an antigen binding fragment thereof comprises CDRs of any antibody or fragment thereof provided herein, for example as described in Tables 5-6, and 14-15.

The determination of percent identity between two sequences (e.g., amino acid sequences or nucleic acid sequences) can be accomplished using a mathematical algorithm. A non-limiting example of a mathematical algorithm utilized for the comparison of two sequences is the algorithm of Karlin and Altschul, Proc. Natl. Acad. Sci. U.S.A. 87:2264 2268 (1990), modified as in Karlin and Altschul, Proc. Natl. Acad. Sci. U.S.A. 90:5873 5877 (1993). Such an algorithm is incorporated into the NBLAST and XBLAST programs of Altschul et al., J. Mol. Biol. 215:403 (1990). BLAST nucleotide searches can be performed with the NBLAST nucleotide program parameters set, e.g., for score=100, word length=12 to obtain nucleotide sequences homologous to a nucleic acid molecule described herein. BLAST protein searches can be performed with the XBLAST program parameters set, e.g., to score 50, word length=3 to obtain amino acid sequences homologous to a protein molecule described herein. To obtain gapped alignments for comparison purposes, Gapped BLAST can be utilized as described in Altschul et al., Nucleic Acids Res. 25:3389 3402 (1997). In some embodiments, the percent identity between two sequences is calculated by dividing the number of residue(s) varied (excluding or including conservative amino acid substitution(s) or degenerate nucleotide substitution(s)) between the two sequences in the alignment with the residue number of any one of the following: (i) full length of the shorter sequence, (ii) full length of the longer sequence, (iii) mean length of the two sequences, (iv) total length of the non-gap portion of the alignment, (v) length of the alignment excluding overhangs, or (vi) length of the alignment including overhangs. Overhangs as used herein with respect to a sequence alignment refer to either or both ends of the alignment where residues of one sequence are considered as aligning to no residues (i.e., gap) in the other sequence. Alternatively, PSI BLAST can be used to perform an iterated search which detects distant relationships between molecules (Id.). When utilizing BLAST, Gapped BLAST, and PSI Blast programs, the default parameters of the respective programs (e.g., of XBLAST and NBLAST) can be used (see, e.g., National Center for Biotechnology Information (NCBI) on the worldwide web, ncbi.nlm.nih.gov). Another non-limiting example of a mathematical algorithm utilized for the comparison of sequences is the algorithm of Myers and Miller, CABIOS 4:11-17 (1998). Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

In some embodiments, the antibody described herein or an antigen binding fragment thereof provided herein contains substitutions (e.g., conservative substitutions), insertions, or deletions relative to the reference sequence, but the antibody comprising that sequence retains the ability to bind to LILRB1. In some embodiments, a total of 1 to 10 amino acids have been substituted, inserted and/or deleted in a reference amino acid sequence. In some embodiments, substitutions, insertions, or deletions occur in regions outside the CDRs (i.e., in the FRs and/or constant regions).

In some embodiments, the position of one or more CDRs along the VH (e.g., CDR1, CDR2, or CDR3) and/or VL (e.g., CDR1, CDR2, or CDR3) region of an LILRB1 binding domain described herein may vary by one, two, three, four, five, or six amino acid positions so long as binding to LILRB1 (e.g., human LILRB1) is maintained (e.g., substantially maintained, for example, at least 50%, at least 60%, at least 70%, at least 80%, at least 90%, or at least 95%). For example, in some embodiments, the position defining a CDR of any of Tables 5-6, and 14-15 may vary by shifting the N-terminal and/or C-terminal boundary of the CDR by one, two, three, four, five, or six amino acids, relative to the current CDR position, so long as binding to LILRB1 (e.g., human LILRB1) is maintained (e.g., substantially maintained, for example, at least 50%, at least 60%, at least 70%, at least 80%, at least 90%, or at least 95%). Additionally or alternatively, in some embodiments, the length of one or more CDRs along the VH (e.g., CDR1, CDR2, or CDR3) and/or VL (e.g., CDR1, CDR2, or CDR3) region of an LILRB1 binding domain described herein may vary (e.g., be shorter or longer) by one, two, three, four, five, or more amino acids, so long as binding to LILRB1 (e.g., human LILRB1) is maintained (e.g., substantially maintained, for example, at least 50%, at least 60%, at least 70%, at least 80%, at least 90%, or at least 95%). For example, in some embodiments, a VH and/or VL CDR1, CDR2, and/or CDR3 described herein may be one, two, three, four, five or more amino acids shorter than one or more of the CDRs described by SEQ ID NOS: 495-848, 1179 and 1281 so long as binding to LILRB1 (e.g., human LILRB1) is maintained (e.g., substantially maintained, for example, at least 50%, at least 60%, at least 70%, at least 80%, at least 90%, or at least 95%). In other embodiments, a VH and/or VL CDR1, CDR2, and/or CDR3 described herein may be one, two, three, four, five or more amino acids longer than one or more of the CDRs described by SEQ ID NOS: 495-848, 1179 and 1281, so long as binding to LILRB1 (e.g., human LILRB1) is maintained (e.g., substantially maintained, for example, at least 50%, at least 60%, at least 70%, at least 80%, at least 90%, or at least 95%). In some embodiments, the amino terminus of a VH and/or VL CDR1, CDR2, and/or CDR3 described herein may be extended or shortened by one, two, three, four, five or more amino acids compared to one or more of the CDRs described by SEQ ID NOS: 495-848, 1179 and 1281, so long as binding to LILRB1 (e.g., human LILRB1) is maintained (e.g., substantially maintained, for example, at least 50%, at least 60%, at least 70%, at least 80%, at least 90%, or at least 95%). Additionally or alternatively, in some embodiments, the carboxy terminus of a VH and/or VL CDR1, CDR2, and/or CDR3 described herein may be extended or shortened by one, two, three, four, five or more amino acids compared to one or more of the CDRs described by SEQ ID NOS: 495-848, 1179 and 1281, so long as binding to LILRB1 (e.g., human LILRB1) is maintained (e.g., substantially maintained, for example, at least 50%, at least 60%, at least 70%, at least 80%, at least 90%, or at least 95%). Any method known in the art can be used to ascertain whether binding to LILRB1 (e.g., human LILRB1) is maintained, for example, the binding assays and conditions described in the “Examples” section described herein.

In other embodiments, the antibody described herein or an antigen binding fragment thereof further comprise conservative sequence modifications (e.g., in an LILRB1 binding domain). Conservative sequence modifications include conservative amino acid substitutions that include ones in which the amino acid residue is replaced with an amino acid residue having a similar side chain. Families of amino acid residues having similar side chains have been defined in the art. Thus, in some embodiments, a predicted nonessential amino acid residue in an LILRB1 is replaced with another amino acid residue from the same side chain family. Methods of identifying amino acid conservative substitutions which do not eliminate antigen binding and nucleotides encoding thereof are well-known in the art (see, e.g., Brummell et al., Biochem. 32:1180-1187 (1993); Kobayashi et al. Protein Eng. 12 (10): 879-884 (1999); and Burks et al. Proc. Natl. Acad. Sci. USA 94:412-417 (1997)). In some embodiments, the conservative sequence modifications described herein modify the amino acid sequences of the binding agents (e.g., antibodies), including human LILRB1 binding agents, by 50%, or 55%, or 60%, or 65%, or 70%, or 75%, or 80%, or 85%, or 90%, or 95%, or 98%, or 99%. In some embodiments, the amino acid sequence modifications refer to at most 1, 2, 3, 4, 5, or 6 amino acid substitutions to the CDRs, such as those described in any one of Tables 5-6, and 14-15. Thus, for example, each such CDR may contain up to 5 conservative amino acid substitutions, for example up to (not more than) 4 conservative amino acid substitutions, for example up to (not more than) 3 conservative amino acid substitutions, for example up to (not more than) 2 conservative amino acid substitutions, or no more than 1 conservative amino acid substitution. In some embodiments, the LILRB1 binding domain contains one or more, including six, CDRs having at least 75%, 80%, 85%, 90%, 95%, 96%, 97%, 98%, or 99% identity to the CDRs of any antibody or fragment thereof provided herein, for example in Tables 5-6, and 14-15.

In some embodiments, an LILRB1 binding domain contains a VH and a VL comprising CDRs identical to any antibody or fragment thereof provided herein, for example in Tables 5-6, and 14-15. In some embodiments, the amino acid sequence modifications do not include any modification within an SDR. In some embodiments, the amino acid sequence modifications do not include any modification within a CDR (such as CDR1, CDR2, CDR3, or any combination thereof). In further embodiments, the amino acid sequence modifications are in the framework or constant region.

In some embodiments, the antibody provide herein contains substitutions (e.g., conservative substitutions), insertions, or deletions relative to the reference sequence, but the anti-LILRB1 antibody comprising that sequence retains the ability to bind to LILRB1. In some embodiments, a total of 1 to 10 amino acids have been substituted, inserted and/or deleted in a reference amino acid sequence. In some embodiments, substitutions, insertions, or deletions occur in regions outside the CDRs (i.e., in the FRs). Optionally, the anti-LILRB1 antibody provided herein includes post-translational modifications of a reference sequence.

In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 283, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 284. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 285, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 286. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 287, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 288. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 289, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 290. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 291, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 292. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 293, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 294. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 295, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 296. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 297, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 298. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 299, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 300. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 301, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 302. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 303, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 304. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 305, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 306. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 307, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 308. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 309, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 310. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 311, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 312. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 313, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 314. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 315, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 316. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 317, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 318. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 319, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 320. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 321, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 322. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 323, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 324. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 325, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 326. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 327, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 328. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 329, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 330. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 331, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 332. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 333, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 334. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 335, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 336. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 337, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 338. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 339, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 340. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 341, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 342. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 343, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 344. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 345, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 346. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 347, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 348. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 349, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 350. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 351, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 352. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 353, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 354. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 355, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 356. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 357, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 358. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 359, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 360. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 361, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 362. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 363, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 364. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 365, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 366. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 367, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 368. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 369, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 370. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 371, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 372. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 373, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 374. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 375, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 376. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 377, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 378. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 379, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 380. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 381, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 382. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 383, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 384. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 385, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 386. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 387, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 388. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 389, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 390. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 391, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 392. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 393, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 394. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 395, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 396. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 397, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 398. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 399, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 400. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 1155, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 1156. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 1157, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 1158. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 1159, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 1160. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 1161, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 1162. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 1163, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 1164. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 1165, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 1166. In all the embodiments described above, the antibodies bind to LILRB1.

In some embodiments, functional epitopes can be mapped, e.g., by combinatorial alanine scanning, to identify amino acids in the LILRB1 protein that are necessary for interaction with anti-LILRB1 antibodies provided herein. In some embodiments, conformational and crystal structure of anti-LILRB1 antibody bound to LILRB1 may be employed to identify the epitopes. In some embodiments, the present disclosure provides an antibody that specifically binds to the same epitope as any of the anti-LILRB1 antibodies provided herein. For example, in some embodiments, the antibody or antigen binding fragment provided herein binds to the same epitope as an anti-LILRB1 antibody comprising a VH comprising the amino acid sequence of SEQ ID NO: 283, and a VL comprising the amino acid sequence of SEQ ID NO: 284. In some embodiments, the antibody or antigen binding fragment provided herein binds to the same epitope as an anti-LILRB1 antibody comprising a VH comprising the amino acid sequence of SEQ ID NO: 285, and a VL comprising the amino acid sequence of SEQ ID NO: 286. In some embodiments, the antibody or antigen binding fragment provided herein binds to the same epitope as an anti-LILRB1 antibody comprising a VH comprising the amino acid sequence of SEQ ID NO: 287, and a VL comprising the amino acid sequence of SEQ ID NO: 288. In some embodiments, the antibody or antigen binding fragment provided herein binds to the same epitope as an anti-LILRB1 antibody comprising a VH comprising the amino acid sequence of SEQ ID NO: 289, and a VL comprising the amino acid sequence of SEQ ID NO: 290. In some embodiments, the antibody or antigen binding fragment provided herein binds to the same epitope as an anti-LILRB1 antibody comprising a VH comprising the amino acid sequence of SEQ ID NO: 291, and a VL comprising the amino acid sequence of SEQ ID NO: 292. In some embodiments, the antibody or antigen binding fragment provided herein binds to the same epitope as an anti-LILRB1 antibody comprising a VH comprising the amino acid sequence of SEQ ID NO: 293, and a VL comprising the amino acid sequence of SEQ ID NO: 294. In some embodiments, the antibody or antigen binding fragment provided herein binds to the same epitope as an anti-LILRB1 antibody comprising a VH comprising the amino acid sequence of SEQ ID NO: 295, and a VL comprising the amino acid sequence of SEQ ID NO: 296. In some embodiments, the antibody or antigen binding fragment provided herein binds to the same epitope as an anti-LILRB1 antibody comprising a VH comprising the amino acid sequence of SEQ ID NO: 297, and a VL comprising the amino acid sequence of SEQ ID NO: 298. In some embodiments, the antibody or antigen binding fragment provided herein binds to the same epitope as an anti-LILRB1 antibody comprising a VH comprising the amino acid sequence of SEQ ID NO: 299, and a VL comprising the amino acid sequence of SEQ ID NO: 300. In some embodiments, the antibody or antigen binding fragment provided herein binds to the same epitope as an anti-LILRB1 antibody comprising a VH comprising the amino acid sequence of SEQ ID NO: 301, and a VL comprising the amino acid sequence of SEQ ID NO: 302. In some embodiments, the antibody or antigen binding fragment provided herein binds to the same epitope as an anti-LILRB1 antibody comprising a VH comprising the amino acid sequence of SEQ ID NO: 303, and a VL comprising the amino acid sequence of SEQ ID NO: 304. In some embodiments, the antibody or antigen binding fragment provided herein binds to the same epitope as an anti-LILRB1 antibody comprising a VH comprising the amino acid sequence of SEQ ID NO: 305, and a VL comprising the amino acid sequence of SEQ ID NO: 306. In some embodiments, the antibody or antigen binding fragment provided herein binds to the same epitope as an anti-LILRB1 antibody comprising a VH comprising the amino acid sequence of SEQ ID NO: 307, and a VL comprising the amino acid sequence of SEQ ID NO: 308. In some embodiments, the antibody or antigen binding fragment provided herein binds to the same epitope as an anti-LILRB1 antibody comprising a VH comprising the amino acid sequence of SEQ ID NO: 309, and a VL comprising the amino acid sequence of SEQ ID NO: 310. In some embodiments, the antibody or antigen binding fragment provided herein binds to the same epitope as an anti-LILRB1 antibody comprising a VH comprising the amino acid sequence of SEQ ID NO: 311, and a VL comprising the amino acid sequence of SEQ ID NO: 312. In some embodiments, the antibody or antigen binding fragment provided herein binds to the same epitope as an anti-LILRB1 antibody comprising a VH comprising the amino acid sequence of SEQ ID NO: 313, and a VL comprising the amino acid sequence of SEQ ID NO: 314. In some embodiments, the antibody or antigen binding fragment provided herein binds to the same epitope as an anti-LILRB1 antibody comprising a VH comprising the amino acid sequence of SEQ ID NO: 315, and a VL comprising the amino acid sequence of SEQ ID NO: 316. In some embodiments, the antibody or antigen binding fragment provided herein binds to the same epitope as an anti-LILRB1 antibody comprising a VH comprising the amino acid sequence of SEQ ID NO: 317, and a VL comprising the amino acid sequence of SEQ ID NO: 318. In some embodiments, the antibody or antigen binding fragment provided herein binds to the same epitope as an anti-LILRB1 antibody comprising a VH comprising the amino acid sequence of SEQ ID NO: 319, and a VL comprising the amino acid sequence of SEQ ID NO: 320. In some embodiments, the antibody or antigen binding fragment provided herein binds to the same epitope as an anti-LILRB1 antibody comprising a VH comprising the amino acid sequence of SEQ ID NO: 321, and a VL comprising the amino acid sequence of SEQ ID NO: 322. In some embodiments, the antibody or antigen binding fragment provided herein binds to the same epitope as an anti-LILRB1 antibody comprising a VH comprising the amino acid sequence of SEQ ID NO: 323, and a VL comprising the amino acid sequence of SEQ ID NO: 324. In some embodiments, the antibody or antigen binding fragment provided herein binds to the same epitope as an anti-LILRB1 antibody comprising a VH comprising the amino acid sequence of SEQ ID NO: 325, and a VL comprising the amino acid sequence of SEQ ID NO: 326. In some embodiments, the antibody or antigen binding fragment provided herein binds to the same epitope as an anti-LILRB1 antibody comprising a VH comprising the amino acid sequence of SEQ ID NO: 327, and a VL comprising the amino acid sequence of SEQ ID NO: 328. In some embodiments, the antibody or antigen binding fragment provided herein binds to the same epitope as an anti-LILRB1 antibody comprising a VH comprising the amino acid sequence of SEQ ID NO: 329, and a VL comprising the amino acid sequence of SEQ ID NO: 330. In some embodiments, the antibody or antigen binding fragment provided herein binds to the same epitope as an anti-LILRB1 antibody comprising a VH comprising the amino acid sequence of SEQ ID NO: 331, and a VL comprising the amino acid sequence of SEQ ID NO: 332. In some embodiments, the antibody or antigen binding fragment provided herein binds to the same epitope as an anti-LILRB1 antibody comprising a VH comprising the amino acid sequence of SEQ ID NO: 333, and a VL comprising the amino acid sequence of SEQ ID NO: 334. In some embodiments, the antibody or antigen binding fragment provided herein binds to the same epitope as an anti-LILRB1 antibody comprising a VH comprising the amino acid sequence of SEQ ID NO: 335, and a VL comprising the amino acid sequence of SEQ ID NO: 336. In some embodiments, the antibody or antigen binding fragment provided herein binds to the same epitope as an anti-LILRB1 antibody comprising a VH comprising the amino acid sequence of SEQ ID NO: 337, and a VL comprising the amino acid sequence of SEQ ID NO: 338. In some embodiments, the antibody or antigen binding fragment provided herein binds to the same epitope as an anti-LILRB1 antibody comprising a VH comprising the amino acid sequence of SEQ ID NO: 339, and a VL comprising the amino acid sequence of SEQ ID NO: 340. In some embodiments, the antibody or antigen binding fragment provided herein binds to the same epitope as an anti-LILRB1 antibody comprising a VH comprising the amino acid sequence of SEQ ID NO: 341, and a VL comprising the amino acid sequence of SEQ ID NO: 342. In some embodiments, the antibody or antigen binding fragment provided herein binds to the same epitope as an anti-LILRB1 antibody comprising a VH comprising the amino acid sequence of SEQ ID NO: 343, and a VL comprising the amino acid sequence of SEQ ID NO: 344. In some embodiments, the antibody or antigen binding fragment provided herein binds to the same epitope as an anti-LILRB1 antibody comprising a VH comprising the amino acid sequence of SEQ ID NO: 345, and a VL comprising the amino acid sequence of SEQ ID NO: 346. In some embodiments, the antibody or antigen binding fragment provided herein binds to the same epitope as an anti-LILRB1 antibody comprising a VH comprising the amino acid sequence of SEQ ID NO: 347, and a VL comprising the amino acid sequence of SEQ ID NO: 348. In some embodiments, the antibody or antigen binding fragment provided herein binds to the same epitope as an anti-LILRB1 antibody comprising a VH comprising the amino acid sequence of SEQ ID NO: 349, and a VL comprising the amino acid sequence of SEQ ID NO: 350. In some embodiments, the antibody or antigen binding fragment provided herein binds to the same epitope as an anti-LILRB1 antibody comprising a VH comprising the amino acid sequence of SEQ ID NO: 351, and a VL comprising the amino acid sequence of SEQ ID NO: 352. In some embodiments, the antibody or antigen binding fragment provided herein binds to the same epitope as an anti-LILRB1 antibody comprising a VH comprising the amino acid sequence of SEQ ID NO: 353, and a VL comprising the amino acid sequence of SEQ ID NO: 354. In some embodiments, the antibody or antigen binding fragment provided herein binds to the same epitope as an anti-LILRB1 antibody comprising a VH comprising the amino acid sequence of SEQ ID NO: 355, and a VL comprising the amino acid sequence of SEQ ID NO: 356. In some embodiments, the antibody or antigen binding fragment provided herein binds to the same epitope as an anti-LILRB1 antibody comprising a VH comprising the amino acid sequence of SEQ ID NO: 357, and a VL comprising the amino acid sequence of SEQ ID NO: 358. In some embodiments, the antibody or antigen binding fragment provided herein binds to the same epitope as an anti-LILRB1 antibody comprising a VH comprising the amino acid sequence of SEQ ID NO: 359, and a VL comprising the amino acid sequence of SEQ ID NO: 360. In some embodiments, the antibody or antigen binding fragment provided herein binds to the same epitope as an anti-LILRB1 antibody comprising a VH comprising the amino acid sequence of SEQ ID NO: 361, and a VL comprising the amino acid sequence of SEQ ID NO: 362. In some embodiments, the antibody or antigen binding fragment provided herein binds to the same epitope as an anti-LILRB1 antibody comprising a VH comprising the amino acid sequence of SEQ ID NO: 363, and a VL comprising the amino acid sequence of SEQ ID NO: 364. In some embodiments, the antibody or antigen binding fragment provided herein binds to the same epitope as an anti-LILRB1 antibody comprising a VH comprising the amino acid sequence of SEQ ID NO: 365, and a VL comprising the amino acid sequence of SEQ ID NO: 366. In some embodiments, the antibody or antigen binding fragment provided herein binds to the same epitope as an anti-LILRB1 antibody comprising a VH comprising the amino acid sequence of SEQ ID NO: 367, and a VL comprising the amino acid sequence of SEQ ID NO: 368. In some embodiments, the antibody or antigen binding fragment provided herein binds to the same epitope as an anti-LILRB1 antibody comprising a VH comprising the amino acid sequence of SEQ ID NO: 369, and a VL comprising the amino acid sequence of SEQ ID NO: 370. In some embodiments, the antibody or antigen binding fragment provided herein binds to the same epitope as an anti-LILRB1 antibody comprising a VH comprising the amino acid sequence of SEQ ID NO: 371, and a VL comprising the amino acid sequence of SEQ ID NO: 372. In some embodiments, the antibody or antigen binding fragment provided herein binds to the same epitope as an anti-LILRB1 antibody comprising a VH comprising the amino acid sequence of SEQ ID NO: 373, and a VL comprising the amino acid sequence of SEQ ID NO: 374. In some embodiments, the antibody or antigen binding fragment provided herein binds to the same epitope as an anti-LILRB1 antibody comprising a VH comprising the amino acid sequence of SEQ ID NO: 375, and a VL comprising the amino acid sequence of SEQ ID NO: 376. In some embodiments, the antibody or antigen binding fragment provided herein binds to the same epitope as an anti-LILRB1 antibody comprising a VH comprising the amino acid sequence of SEQ ID NO: 377, and a VL comprising the amino acid sequence of SEQ ID NO: 378. In some embodiments, the antibody or antigen binding fragment provided herein binds to the same epitope as an anti-LILRB1 antibody comprising a VH comprising the amino acid sequence of SEQ ID NO: 379, and a VL comprising the amino acid sequence of SEQ ID NO: 380. In some embodiments, the antibody or antigen binding fragment provided herein binds to the same epitope as an anti-LILRB1 antibody comprising a VH comprising the amino acid sequence of SEQ ID NO: 381, and a VL comprising the amino acid sequence of SEQ ID NO: 382. In some embodiments, the antibody or antigen binding fragment provided herein binds to the same epitope as an anti-LILRB1 antibody comprising a VH comprising the amino acid sequence of SEQ ID NO: 383, and a VL comprising the amino acid sequence of SEQ ID NO: 384. In some embodiments, the antibody or antigen binding fragment provided herein binds to the same epitope as an anti-LILRB1 antibody comprising a VH comprising the amino acid sequence of SEQ ID NO: 385, and a VL comprising the amino acid sequence of SEQ ID NO: 386. In some embodiments, the antibody or antigen binding fragment provided herein binds to the same epitope as an anti-LILRB1 antibody comprising a VH comprising the amino acid sequence of SEQ ID NO: 387, and a VL comprising the amino acid sequence of SEQ ID NO: 388. In some embodiments, the antibody or antigen binding fragment provided herein binds to the same epitope as an anti-LILRB1 antibody comprising a VH comprising the amino acid sequence of SEQ ID NO: 389, and a VL comprising the amino acid sequence of SEQ ID NO: 390. In some embodiments, the antibody or antigen binding fragment provided herein binds to the same epitope as an anti-LILRB1 antibody comprising a VH comprising the amino acid sequence of SEQ ID NO: 391, and a VL comprising the amino acid sequence of SEQ ID NO: 392. In some embodiments, the antibody or antigen binding fragment provided herein binds to the same epitope as an anti-LILRB1 antibody comprising a VH comprising the amino acid sequence of SEQ ID NO: 393, and a VL comprising the amino acid sequence of SEQ ID NO: 394. In some embodiments, the antibody or antigen binding fragment provided herein binds to the same epitope as an anti-LILRB1 antibody comprising a VH comprising the amino acid sequence of SEQ ID NO: 395, and a VL comprising the amino acid sequence of SEQ ID NO: 396. In some embodiments, the antibody or antigen binding fragment provided herein binds to the same epitope as an anti-LILRB1 antibody comprising a VH comprising the amino acid sequence of SEQ ID NO: 397, and a VL comprising the amino acid sequence of SEQ ID NO: 398. In some embodiments, the antibody or antigen binding fragment provided herein binds to the same epitope as an anti-LILRB1 antibody comprising a VH comprising the amino acid sequence of SEQ ID NO: 399, and a VL comprising the amino acid sequence of SEQ ID NO: 400. In some embodiments, the antibody or antigen binding fragment provided herein binds to the same epitope as an anti-LILRB1 antibody comprising a VH comprising the amino acid sequence of SEQ ID NO: 1155, and a VL comprising the amino acid sequence of SEQ ID NO: 1156. In some embodiments, the antibody or antigen binding fragment provided herein binds to the same epitope as an anti-LILRB1 antibody comprising a VH comprising the amino acid sequence of SEQ ID NO: 1157, and a VL comprising the amino acid sequence of SEQ ID NO: 1158. In some embodiments, the antibody or antigen binding fragment provided herein binds to the same epitope as an anti-LILRB1 antibody comprising a VH comprising the amino acid sequence of SEQ ID NO: 1159, and a VL comprising the amino acid sequence of SEQ ID NO: 1160. In some embodiments, the antibody or antigen binding fragment provided herein binds to the same epitope as an anti-LILRB1 antibody comprising a VH comprising the amino acid sequence of SEQ ID NO: 1161, and a VL comprising the amino acid sequence of SEQ ID NO: 1162. In some embodiments, the antibody or antigen binding fragment provided herein binds to the same epitope as an anti-LILRB1 antibody comprising a VH comprising the amino acid sequence of SEQ ID NO: 1163, and a VL comprising the amino acid sequence of SEQ ID NO: 1164. In some embodiments, the antibody or antigen binding fragment provided herein binds to the same epitope as an anti-LILRB1 antibody comprising a VH comprising the amino acid sequence of SEQ ID NO: 1165, and a VL comprising the amino acid sequence of SEQ ID NO: 1166.

In some embodiments, provided herein is an anti-LILRB1 antibody, or antigen binding fragment thereof, that specifically binds to LILRB1 competitively with any one of the anti-LILRB1 antibodies described herein. In some embodiments, the antibody or antigen binding fragment provided herein specifically binds to LILRB1 competitively with an anti-LILRB1 antibody comprising a VH comprising the amino acid sequence of SEQ ID NO: 283, and a VL comprising the amino acid sequence of SEQ ID NO: 284. In some embodiments, the antibody or antigen binding fragment provided herein specifically binds to LILRB1 competitively with an anti-LILRB1 antibody comprising a VH comprising the amino acid sequence of SEQ ID NO: 285, and a VL comprising the amino acid sequence of SEQ ID NO: 286. In some embodiments, the antibody or antigen binding fragment provided herein specifically binds to LILRB1 competitively with an anti-LILRB1 antibody comprising a VH comprising the amino acid sequence of SEQ ID NO: 287, and a VL comprising the amino acid sequence of SEQ ID NO: 288. In some embodiments, the antibody or antigen binding fragment provided herein specifically binds to LILRB1 competitively with an anti-LILRB1 antibody comprising a VH comprising the amino acid sequence of SEQ ID NO: 289, and a VL comprising the amino acid sequence of SEQ ID NO: 290. In some embodiments, the antibody or antigen binding fragment provided herein specifically binds to LILRB1 competitively with an anti-LILRB1 antibody comprising a VH comprising the amino acid sequence of SEQ ID NO: 291, and a VL comprising the amino acid sequence of SEQ ID NO: 292. In some embodiments, the antibody or antigen binding fragment provided herein specifically binds to LILRB1 competitively with an anti-LILRB1 antibody comprising a VH comprising the amino acid sequence of SEQ ID NO: 293, and a VL comprising the amino acid sequence of SEQ ID NO: 294. In some embodiments, the antibody or antigen binding fragment provided herein specifically binds to LILRB1 competitively with an anti-LILRB1 antibody comprising a VH comprising the amino acid sequence of SEQ ID NO: 295, and a VL comprising the amino acid sequence of SEQ ID NO: 296. In some embodiments, the antibody or antigen binding fragment provided herein specifically binds to LILRB1 competitively with an anti-LILRB1 antibody comprising a VH comprising the amino acid sequence of SEQ ID NO: 297, and a VL comprising the amino acid sequence of SEQ ID NO: 298. In some embodiments, the antibody or antigen binding fragment provided herein specifically binds to LILRB1 competitively with an anti-LILRB1 antibody comprising a VH comprising the amino acid sequence of SEQ ID NO: 299, and a VL comprising the amino acid sequence of SEQ ID NO: 300. In some embodiments, the antibody or antigen binding fragment provided herein specifically binds to LILRB1 competitively with an anti-LILRB1 antibody comprising a VH comprising the amino acid sequence of SEQ ID NO: 301, and a VL comprising the amino acid sequence of SEQ ID NO: 302. In some embodiments, the antibody or antigen binding fragment provided herein specifically binds to LILRB1 competitively with an anti-LILRB1 antibody comprising a VH comprising the amino acid sequence of SEQ ID NO: 303, and a VL comprising the amino acid sequence of SEQ ID NO: 304. In some embodiments, the antibody or antigen binding fragment provided herein specifically binds to LILRB1 competitively with an anti-LILRB1 antibody comprising a VH comprising the amino acid sequence of SEQ ID NO: 305, and a VL comprising the amino acid sequence of SEQ ID NO: 306. In some embodiments, the antibody or antigen binding fragment provided herein specifically binds to LILRB1 competitively with an anti-LILRB1 antibody comprising a VH comprising the amino acid sequence of SEQ ID NO: 307, and a VL comprising the amino acid sequence of SEQ ID NO: 308. In some embodiments, the antibody or antigen binding fragment provided herein specifically binds to LILRB1 competitively with an anti-LILRB1 antibody comprising a VH comprising the amino acid sequence of SEQ ID NO: 309, and a VL comprising the amino acid sequence of SEQ ID NO: 310. In some embodiments, the antibody or antigen binding fragment provided herein specifically binds to LILRB1 competitively with an anti-LILRB1 antibody comprising a VH comprising the amino acid sequence of SEQ ID NO: 311, and a VL comprising the amino acid sequence of SEQ ID NO: 312. In some embodiments, the antibody or antigen binding fragment provided herein specifically binds to LILRB1 competitively with an anti-LILRB1 antibody comprising a VH comprising the amino acid sequence of SEQ ID NO: 313, and a VL comprising the amino acid sequence of SEQ ID NO: 314. In some embodiments, the antibody or antigen binding fragment provided herein specifically binds to LILRB1 competitively with an anti-LILRB1 antibody comprising a VH comprising the amino acid sequence of SEQ ID NO: 315, and a VL comprising the amino acid sequence of SEQ ID NO: 316. In some embodiments, the antibody or antigen binding fragment provided herein specifically binds to LILRB1 competitively with an anti-LILRB1 antibody comprising a VH comprising the amino acid sequence of SEQ ID NO: 317, and a VL comprising the amino acid sequence of SEQ ID NO: 318. In some embodiments, the antibody or antigen binding fragment provided herein specifically binds to LILRB1 competitively with an anti-LILRB1 antibody comprising a VH comprising the amino acid sequence of SEQ ID NO: 319, and a VL comprising the amino acid sequence of SEQ ID NO: 320. In some embodiments, the antibody or antigen binding fragment provided herein specifically binds to LILRB1 competitively with an anti-LILRB1 antibody comprising a VH comprising the amino acid sequence of SEQ ID NO: 321, and a VL comprising the amino acid sequence of SEQ ID NO: 322. In some embodiments, the antibody or antigen binding fragment provided herein specifically binds to LILRB1 competitively with an anti-LILRB1 antibody comprising a VH comprising the amino acid sequence of SEQ ID NO: 323, and a VL comprising the amino acid sequence of SEQ ID NO: 324. In some embodiments, the antibody or antigen binding fragment provided herein specifically binds to LILRB1 competitively with an anti-LILRB1 antibody comprising a VH comprising the amino acid sequence of SEQ ID NO: 325, and a VL comprising the amino acid sequence of SEQ ID NO: 326. In some embodiments, the antibody or antigen binding fragment provided herein specifically binds to LILRB1 competitively with an anti-LILRB1 antibody comprising a VH comprising the amino acid sequence of SEQ ID NO: 327, and a VL comprising the amino acid sequence of SEQ ID NO: 328. In some embodiments, the antibody or antigen binding fragment provided herein specifically binds to LILRB1 competitively with an anti-LILRB1 antibody comprising a VH comprising the amino acid sequence of SEQ ID NO: 329, and a VL comprising the amino acid sequence of SEQ ID NO: 330. In some embodiments, the antibody or antigen binding fragment provided herein specifically binds to LILRB1 competitively with an anti-LILRB1 antibody comprising a VH comprising the amino acid sequence of SEQ ID NO: 331, and a VL comprising the amino acid sequence of SEQ ID NO: 332. In some embodiments, the antibody or antigen binding fragment provided herein specifically binds to LILRB1 competitively with an anti-LILRB1 antibody comprising a VH comprising the amino acid sequence of SEQ ID NO: 333, and a VL comprising the amino acid sequence of SEQ ID NO: 334. In some embodiments, the antibody or antigen binding fragment provided herein specifically binds to LILRB1 competitively with an anti-LILRB1 antibody comprising a VH comprising the amino acid sequence of SEQ ID NO: 335, and a VL comprising the amino acid sequence of SEQ ID NO: 336. In some embodiments, the antibody or antigen binding fragment provided herein specifically binds to LILRB1 competitively with an anti-LILRB1 antibody comprising a VH comprising the amino acid sequence of SEQ ID NO: 337, and a VL comprising the amino acid sequence of SEQ ID NO: 338. In some embodiments, the antibody or antigen binding fragment provided herein specifically binds to LILRB1 competitively with an anti-LILRB1 antibody comprising a VH comprising the amino acid sequence of SEQ ID NO: 339, and a VL comprising the amino acid sequence of SEQ ID NO: 340. In some embodiments, the antibody or antigen binding fragment provided herein specifically binds to LILRB1 competitively with an anti-LILRB1 antibody comprising a VH comprising the amino acid sequence of SEQ ID NO: 341, and a VL comprising the amino acid sequence of SEQ ID NO: 342. In some embodiments, the antibody or antigen binding fragment provided herein specifically binds to LILRB1 competitively with an anti-LILRB1 antibody comprising a VH comprising the amino acid sequence of SEQ ID NO: 343, and a VL comprising the amino acid sequence of SEQ ID NO: 344. In some embodiments, the antibody or antigen binding fragment provided herein specifically binds to LILRB1 competitively with an anti-LILRB1 antibody comprising a VH comprising the amino acid sequence of SEQ ID NO: 345, and a VL comprising the amino acid sequence of SEQ ID NO: 346. In some embodiments, the antibody or antigen binding fragment provided herein specifically binds to LILRB1 competitively with an anti-LILRB1 antibody comprising a VH comprising the amino acid sequence of SEQ ID NO: 347, and a VL comprising the amino acid sequence of SEQ ID NO: 348. In some embodiments, the antibody or antigen binding fragment provided herein specifically binds to LILRB1 competitively with an anti-LILRB1 antibody comprising a VH comprising the amino acid sequence of SEQ ID NO: 349, and a VL comprising the amino acid sequence of SEQ ID NO: 350. In some embodiments, the antibody or antigen binding fragment provided herein specifically binds to LILRB1 competitively with an anti-LILRB1 antibody comprising a VH comprising the amino acid sequence of SEQ ID NO: 351, and a VL comprising the amino acid sequence of SEQ ID NO: 352. In some embodiments, the antibody or antigen binding fragment provided herein specifically binds to LILRB1 competitively with an anti-LILRB1 antibody comprising a VH comprising the amino acid sequence of SEQ ID NO: 353, and a VL comprising the amino acid sequence of SEQ ID NO: 354. In some embodiments, the antibody or antigen binding fragment provided herein specifically binds to LILRB1 competitively with an anti-LILRB1 antibody comprising a VH comprising the amino acid sequence of SEQ ID NO: 355, and a VL comprising the amino acid sequence of SEQ ID NO: 356. In some embodiments, the antibody or antigen binding fragment provided herein specifically binds to LILRB1 competitively with an anti-LILRB1 antibody comprising a VH comprising the amino acid sequence of SEQ ID NO: 357, and a VL comprising the amino acid sequence of SEQ ID NO: 358. In some embodiments, the antibody or antigen binding fragment provided herein specifically binds to LILRB1 competitively with an anti-LILRB1 antibody comprising a VH comprising the amino acid sequence of SEQ ID NO: 359, and a VL comprising the amino acid sequence of SEQ ID NO: 360. In some embodiments, the antibody or antigen binding fragment provided herein specifically binds to LILRB1 competitively with an anti-LILRB1 antibody comprising a VH comprising the amino acid sequence of SEQ ID NO: 361, and a VL comprising the amino acid sequence of SEQ ID NO: 362. In some embodiments, the antibody or antigen binding fragment provided herein specifically binds to LILRB1 competitively with an anti-LILRB1 antibody comprising a VH comprising the amino acid sequence of SEQ ID NO: 363, and a VL comprising the amino acid sequence of SEQ ID NO: 364. In some embodiments, the antibody or antigen binding fragment provided herein specifically binds to LILRB1 competitively with an anti-LILRB1 antibody comprising a VH comprising the amino acid sequence of SEQ ID NO: 365, and a VL comprising the amino acid sequence of SEQ ID NO: 366. In some embodiments, the antibody or antigen binding fragment provided herein specifically binds to LILRB1 competitively with an anti-LILRB1 antibody comprising a VH comprising the amino acid sequence of SEQ ID NO: 367, and a VL comprising the amino acid sequence of SEQ ID NO: 368. In some embodiments, the antibody or antigen binding fragment provided herein specifically binds to LILRB1 competitively with an anti-LILRB1 antibody comprising a VH comprising the amino acid sequence of SEQ ID NO: 369, and a VL comprising the amino acid sequence of SEQ ID NO: 370. In some embodiments, the antibody or antigen binding fragment provided herein specifically binds to LILRB1 competitively with an anti-LILRB1 antibody comprising a VH comprising the amino acid sequence of SEQ ID NO: 371, and a VL comprising the amino acid sequence of SEQ ID NO: 372. In some embodiments, the antibody or antigen binding fragment provided herein specifically binds to LILRB1 competitively with an anti-LILRB1 antibody comprising a VH comprising the amino acid sequence of SEQ ID NO: 373, and a VL comprising the amino acid sequence of SEQ ID NO: 374. In some embodiments, the antibody or antigen binding fragment provided herein specifically binds to LILRB1 competitively with an anti-LILRB1 antibody comprising a VH comprising the amino acid sequence of SEQ ID NO: 375, and a VL comprising the amino acid sequence of SEQ ID NO: 376. In some embodiments, the antibody or antigen binding fragment provided herein specifically binds to LILRB1 competitively with an anti-LILRB1 antibody comprising a VH comprising the amino acid sequence of SEQ ID NO: 377, and a VL comprising the amino acid sequence of SEQ ID NO: 378. In some embodiments, the antibody or antigen binding fragment provided herein specifically binds to LILRB1 competitively with an anti-LILRB1 antibody comprising a VH comprising the amino acid sequence of SEQ ID NO: 379, and a VL comprising the amino acid sequence of SEQ ID NO: 380. In some embodiments, the antibody or antigen binding fragment provided herein specifically binds to LILRB1 competitively with an anti-LILRB1 antibody comprising a VH comprising the amino acid sequence of SEQ ID NO: 381, and a VL comprising the amino acid sequence of SEQ ID NO: 382. In some embodiments, the antibody or antigen binding fragment provided herein specifically binds to LILRB1 competitively with an anti-LILRB1 antibody comprising a VH comprising the amino acid sequence of SEQ ID NO: 383, and a VL comprising the amino acid sequence of SEQ ID NO: 384. In some embodiments, the antibody or antigen binding fragment provided herein specifically binds to LILRB1 competitively with an anti-LILRB1 antibody comprising a VH comprising the amino acid sequence of SEQ ID NO: 385, and a VL comprising the amino acid sequence of SEQ ID NO: 386. In some embodiments, the antibody or antigen binding fragment provided herein specifically binds to LILRB1 competitively with an anti-LILRB1 antibody comprising a VH comprising the amino acid sequence of SEQ ID NO: 387, and a VL comprising the amino acid sequence of SEQ ID NO: 388. In some embodiments, the antibody or antigen binding fragment provided herein specifically binds to LILRB1 competitively with an anti-LILRB1 antibody comprising a VH comprising the amino acid sequence of SEQ ID NO: 389, and a VL comprising the amino acid sequence of SEQ ID NO: 390. In some embodiments, the antibody or antigen binding fragment provided herein specifically binds to LILRB1 competitively with an anti-LILRB1 antibody comprising a VH comprising the amino acid sequence of SEQ ID NO: 391, and a VL comprising the amino acid sequence of SEQ ID NO: 392. In some embodiments, the antibody or antigen binding fragment provided herein specifically binds to LILRB1 competitively with an anti-LILRB1 antibody comprising a VH comprising the amino acid sequence of SEQ ID NO: 393, and a VL comprising the amino acid sequence of SEQ ID NO: 394. In some embodiments, the antibody or antigen binding fragment provided herein specifically binds to LILRB1 competitively with an anti-LILRB1 antibody comprising a VH comprising the amino acid sequence of SEQ ID NO: 395, and a VL comprising the amino acid sequence of SEQ ID NO: 396. In some embodiments, the antibody or antigen binding fragment provided herein specifically binds to LILRB1 competitively with an anti-LILRB1 antibody comprising a VH comprising the amino acid sequence of SEQ ID NO: 397, and a VL comprising the amino acid sequence of SEQ ID NO: 398. In some embodiments, the antibody or antigen binding fragment provided herein specifically binds to LILRB1 competitively with an anti-LILRB1 antibody comprising a VH comprising the amino acid sequence of SEQ ID NO: 399, and a VL comprising the amino acid sequence of SEQ ID NO: 400. In some embodiments, the antibody or antigen binding fragment provided herein specifically binds to LILRB1 competitively with an anti-LILRB1 antibody comprising a VH comprising the amino acid sequence of SEQ ID NO: 1155, and a VL comprising the amino acid sequence of SEQ ID NO: 1156. In some embodiments, the antibody or antigen binding fragment provided herein specifically binds to LILRB1 competitively with an anti-LILRB1 antibody comprising a VH comprising the amino acid sequence of SEQ ID NO: 1157, and a VL comprising the amino acid sequence of SEQ ID NO: 1158. In some embodiments, the antibody or antigen binding fragment provided herein specifically binds to LILRB1 competitively with an anti-LILRB1 antibody comprising a VH comprising the amino acid sequence of SEQ ID NO: 1159, and a VL comprising the amino acid sequence of SEQ ID NO: 1160. In some embodiments, the antibody or antigen binding fragment provided herein specifically binds to LILRB1 competitively with an anti-LILRB1 antibody comprising a VH comprising the amino acid sequence of SEQ ID NO: 1161, and a VL comprising the amino acid sequence of SEQ ID NO: 1162. In some embodiments, the antibody or antigen binding fragment provided herein specifically binds to LILRB1 competitively with an anti-LILRB1 antibody comprising a VH comprising the amino acid sequence of SEQ ID NO: 1163, and a VL comprising the amino acid sequence of SEQ ID NO: 1164. In some embodiments, the antibody or antigen binding fragment provided herein specifically binds to LILRB1 competitively with an anti-LILRB1 antibody comprising a VH comprising the amino acid sequence of SEQ ID NO: 1165, and a VL comprising the amino acid sequence of SEQ ID NO: 1166.

In some embodiments, the antibody or antigen binding fragment provided herein comprises a heavy chain comprising the amino acid sequence of SEQ ID NO: 71, and a light chain comprising the amino acid sequence of SEQ ID NO: 72. In some embodiments, the antibody or antigen binding fragment provided herein comprises a heavy chain comprising the amino acid sequence of SEQ ID NO: 73, and a light chain comprising the amino acid sequence of SEQ ID NO: 74. In some embodiments, the antibody or antigen binding fragment provided herein comprises a heavy chain comprising the amino acid sequence of SEQ ID NO: 75, and a light chain comprising the amino acid sequence of SEQ ID NO: 76. In some embodiments, the antibody or antigen binding fragment provided herein comprises a heavy chain comprising the amino acid sequence of SEQ ID NO: 77, and a light chain comprising the amino acid sequence of SEQ ID NO: 78. In some embodiments, the antibody or antigen binding fragment provided herein comprises a heavy chain comprising the amino acid sequence of SEQ ID NO: 79, and a light chain comprising the amino acid sequence of SEQ ID NO: 80. In some embodiments, the antibody or antigen binding fragment provided herein comprises a heavy chain comprising the amino acid sequence of SEQ ID NO: 81, and a light chain comprising the amino acid sequence of SEQ ID NO: 82. In some embodiments, the antibody or antigen binding fragment provided herein comprises a heavy chain comprising the amino acid sequence of SEQ ID NO: 83, and a light chain comprising the amino acid sequence of SEQ ID NO: 84. In some embodiments, the antibody or antigen binding fragment provided herein comprises a heavy chain comprising the amino acid sequence of SEQ ID NO: 85, and a light chain comprising the amino acid sequence of SEQ ID NO: 86. In some embodiments, the antibody or antigen binding fragment provided herein comprises a heavy chain comprising the amino acid sequence of SEQ ID NO: 87, and a light chain comprising the amino acid sequence of SEQ ID NO: 88. In some embodiments, the antibody or antigen binding fragment provided herein comprises a heavy chain comprising the amino acid sequence of SEQ ID NO: 89, and a light chain comprising the amino acid sequence of SEQ ID NO: 90. In some embodiments, the antibody or antigen binding fragment provided herein comprises a heavy chain comprising the amino acid sequence of SEQ ID NO: 91, and a light chain comprising the amino acid sequence of SEQ ID NO: 92. In some embodiments, the antibody or antigen binding fragment provided herein comprises a heavy chain comprising the amino acid sequence of SEQ ID NO: 93, and a light chain comprising the amino acid sequence of SEQ ID NO: 94. In some embodiments, the antibody or antigen binding fragment provided herein comprises a heavy chain comprising the amino acid sequence of SEQ ID NO: 95, and a light chain comprising the amino acid sequence of SEQ ID NO: 96. In some embodiments, the antibody or antigen binding fragment provided herein comprises a heavy chain comprising the amino acid sequence of SEQ ID NO: 97, and a light chain comprising the amino acid sequence of SEQ ID NO: 98. In some embodiments, the antibody or antigen binding fragment provided herein comprises a heavy chain comprising the amino acid sequence of SEQ ID NO: 99, and a light chain comprising the amino acid sequence of SEQ ID NO: 100. In some embodiments, the antibody or antigen binding fragment provided herein comprises a heavy chain comprising the amino acid sequence of SEQ ID NO: 101, and a light chain comprising the amino acid sequence of SEQ ID NO: 102. In some embodiments, the antibody or antigen binding fragment provided herein comprises a heavy chain comprising the amino acid sequence of SEQ ID NO: 103, and a light chain comprising the amino acid sequence of SEQ ID NO: 104. In some embodiments, the antibody or antigen binding fragment provided herein comprises a heavy chain comprising the amino acid sequence of SEQ ID NO: 105, and a light chain comprising the amino acid sequence of SEQ ID NO: 106. In some embodiments, the antibody or antigen binding fragment provided herein comprises a heavy chain comprising the amino acid sequence of SEQ ID NO: 107, and a light chain comprising the amino acid sequence of SEQ ID NO: 108. In some embodiments, the antibody or antigen binding fragment provided herein comprises a heavy chain comprising the amino acid sequence of SEQ ID NO: 109, and a light chain comprising the amino acid sequence of SEQ ID NO: 110. In some embodiments, the antibody or antigen binding fragment provided herein comprises a heavy chain comprising the amino acid sequence of SEQ ID NO: 111, and a light chain comprising the amino acid sequence of SEQ ID NO: 112. In some embodiments, the antibody or antigen binding fragment provided herein comprises a heavy chain comprising the amino acid sequence of SEQ ID NO: 113, and a light chain comprising the amino acid sequence of SEQ ID NO: 114. In some embodiments, the antibody or antigen binding fragment provided herein comprises a heavy chain comprising the amino acid sequence of SEQ ID NO: 115, and a light chain comprising the amino acid sequence of SEQ ID NO: 116. In some embodiments, the antibody or antigen binding fragment provided herein comprises a heavy chain comprising the amino acid sequence of SEQ ID NO: 117, and a light chain comprising the amino acid sequence of SEQ ID NO: 118. In some embodiments, the antibody or antigen binding fragment provided herein comprises a heavy chain comprising the amino acid sequence of SEQ ID NO: 119, and a light chain comprising the amino acid sequence of SEQ ID NO: 120. In some embodiments, the antibody or antigen binding fragment provided herein comprises a heavy chain comprising the amino acid sequence of SEQ ID NO: 121, and a light chain comprising the amino acid sequence of SEQ ID NO: 122. In some embodiments, the antibody or antigen binding fragment provided herein comprises a heavy chain comprising the amino acid sequence of SEQ ID NO: 123, and a light chain comprising the amino acid sequence of SEQ ID NO: 124. In some embodiments, the antibody or antigen binding fragment provided herein comprises a heavy chain comprising the amino acid sequence of SEQ ID NO: 125, and a light chain comprising the amino acid sequence of SEQ ID NO: 126. In some embodiments, the antibody or antigen binding fragment provided herein comprises a heavy chain comprising the amino acid sequence of SEQ ID NO: 127, and a light chain comprising the amino acid sequence of SEQ ID NO: 128. In some embodiments, the antibody or antigen binding fragment provided herein comprises a heavy chain comprising the amino acid sequence of SEQ ID NO: 129, and a light chain comprising the amino acid sequence of SEQ ID NO: 130. In some embodiments, the antibody or antigen binding fragment provided herein comprises a heavy chain comprising the amino acid sequence of SEQ ID NO: 131, and a light chain comprising the amino acid sequence of SEQ ID NO: 132. In some embodiments, the antibody or antigen binding fragment provided herein comprises a heavy chain comprising the amino acid sequence of SEQ ID NO: 133, and a light chain comprising the amino acid sequence of SEQ ID NO: 134. In some embodiments, the antibody or antigen binding fragment provided herein comprises a heavy chain comprising the amino acid sequence of SEQ ID NO: 135, and a light chain comprising the amino acid sequence of SEQ ID NO: 136. In some embodiments, the antibody or antigen binding fragment provided herein comprises a heavy chain comprising the amino acid sequence of SEQ ID NO: 137, and a light chain comprising the amino acid sequence of SEQ ID NO: 138. In some embodiments, the antibody or antigen binding fragment provided herein comprises a heavy chain comprising the amino acid sequence of SEQ ID NO: 139, and a light chain comprising the amino acid sequence of SEQ ID NO: 140. In some embodiments, the antibody or antigen binding fragment provided herein comprises a heavy chain comprising the amino acid sequence of SEQ ID NO: 141, and a light chain comprising the amino acid sequence of SEQ ID NO: 142. In some embodiments, the antibody or antigen binding fragment provided herein comprises a heavy chain comprising the amino acid sequence of SEQ ID NO: 143, and a light chain comprising the amino acid sequence of SEQ ID NO: 144. In some embodiments, the antibody or antigen binding fragment provided herein comprises a heavy chain comprising the amino acid sequence of SEQ ID NO: 145, and a light chain comprising the amino acid sequence of SEQ ID NO: 146. In some embodiments, the antibody or antigen binding fragment provided herein comprises a heavy chain comprising the amino acid sequence of SEQ ID NO: 147, and a light chain comprising the amino acid sequence of SEQ ID NO: 148. In some embodiments, the antibody or antigen binding fragment provided herein comprises a heavy chain comprising the amino acid sequence of SEQ ID NO: 149, and a light chain comprising the amino acid sequence of SEQ ID NO: 150. In some embodiments, the antibody or antigen binding fragment provided herein comprises a heavy chain comprising the amino acid sequence of SEQ ID NO: 151, and a light chain comprising the amino acid sequence of SEQ ID NO: 152. In some embodiments, the antibody or antigen binding fragment provided herein comprises a heavy chain comprising the amino acid sequence of SEQ ID NO: 153, and a light chain comprising the amino acid sequence of SEQ ID NO: 154. In some embodiments, the antibody or antigen binding fragment provided herein comprises a heavy chain comprising the amino acid sequence of SEQ ID NO: 155, and a light chain comprising the amino acid sequence of SEQ ID NO: 156. In some embodiments, the antibody or antigen binding fragment provided herein comprises a heavy chain comprising the amino acid sequence of SEQ ID NO: 157, and a light chain comprising the amino acid sequence of SEQ ID NO: 158. In some embodiments, the antibody or antigen binding fragment provided herein comprises a heavy chain comprising the amino acid sequence of SEQ ID NO: 159, and a light chain comprising the amino acid sequence of SEQ ID NO: 160. In some embodiments, the antibody or antigen binding fragment provided herein comprises a heavy chain comprising the amino acid sequence of SEQ ID NO: 161, and a light chain comprising the amino acid sequence of SEQ ID NO: 162. In some embodiments, the antibody or antigen binding fragment provided herein comprises a heavy chain comprising the amino acid sequence of SEQ ID NO: 163, and a light chain comprising the amino acid sequence of SEQ ID NO: 164. In some embodiments, the antibody or antigen binding fragment provided herein comprises a heavy chain comprising the amino acid sequence of SEQ ID NO: 165, and a light chain comprising the amino acid sequence of SEQ ID NO: 166. In some embodiments, the antibody or antigen binding fragment provided herein comprises a heavy chain comprising the amino acid sequence of SEQ ID NO: 167, and a light chain comprising the amino acid sequence of SEQ ID NO: 168. In some embodiments, the antibody or antigen binding fragment provided herein comprises a heavy chain comprising the amino acid sequence of SEQ ID NO: 169, and a light chain comprising the amino acid sequence of SEQ ID NO: 170. In some embodiments, the antibody or antigen binding fragment provided herein comprises a heavy chain comprising the amino acid sequence of SEQ ID NO: 171, and a light chain comprising the amino acid sequence of SEQ ID NO: 172. In some embodiments, the antibody or antigen binding fragment provided herein comprises a heavy chain comprising the amino acid sequence of SEQ ID NO: 173, and a light chain comprising the amino acid sequence of SEQ ID NO: 174. In some embodiments, the antibody or antigen binding fragment provided herein comprises a heavy chain comprising the amino acid sequence of SEQ ID NO: 175, and a light chain comprising the amino acid sequence of SEQ ID NO: 176. In some embodiments, the antibody or antigen binding fragment provided herein comprises a heavy chain comprising the amino acid sequence of SEQ ID NO: 177, and a light chain comprising the amino acid sequence of SEQ ID NO: 178. In some embodiments, the antibody or antigen binding fragment provided herein comprises a heavy chain comprising the amino acid sequence of SEQ ID NO: 179, and a light chain comprising the amino acid sequence of SEQ ID NO: 180. In some embodiments, the antibody or antigen binding fragment provided herein comprises a heavy chain comprising the amino acid sequence of SEQ ID NO: 181, and a light chain comprising the amino acid sequence of SEQ ID NO: 182. In some embodiments, the antibody or antigen binding fragment provided herein comprises a heavy chain comprising the amino acid sequence of SEQ ID NO: 183, and a light chain comprising the amino acid sequence of SEQ ID NO: 184. In some embodiments, the antibody or antigen binding fragment provided herein comprises a heavy chain comprising the amino acid sequence of SEQ ID NO: 185, and a light chain comprising the amino acid sequence of SEQ ID NO: 186. In some embodiments, the antibody or antigen binding fragment provided herein comprises a heavy chain comprising the amino acid sequence of SEQ ID NO: 187, and a light chain comprising the amino acid sequence of SEQ ID NO: 188. In some embodiments, the antibody or antigen binding fragment provided herein comprises a heavy chain comprising the amino acid sequence of SEQ ID NO: 1131, and a light chain comprising the amino acid sequence of SEQ ID NO: 1132. In some embodiments, the antibody or antigen binding fragment provided herein comprises a heavy chain comprising the amino acid sequence of SEQ ID NO: 1133, and a light chain comprising the amino acid sequence of SEQ ID NO: 1134. In some embodiments, the antibody or antigen binding fragment provided herein comprises a heavy chain comprising the amino acid sequence of SEQ ID NO: 1135, and a light chain comprising the amino acid sequence of SEQ ID NO: 1136. In some embodiments, the antibody or antigen binding fragment provided herein comprises a heavy chain comprising the amino acid sequence of SEQ ID NO: 1137, and a light chain comprising the amino acid sequence of SEQ ID NO: 1138. In some embodiments, the antibody or antigen binding fragment provided herein comprises a heavy chain comprising the amino acid sequence of SEQ ID NO: 1139, and a light chain comprising the amino acid sequence of SEQ ID NO: 1140. In some embodiments, the antibody or antigen binding fragment provided herein comprises a heavy chain comprising the amino acid sequence of SEQ ID NO: 1141, and a light chain comprising the amino acid sequence of SEQ ID NO: 1142.

In some embodiments, provided herein is a LILRB1 binding protein comprising any one of the anti-LILRB1 antibodies described above. In some embodiments, the LILRB1 binding protein is a monoclonal antibody, including a mouse, chimeric, humanized or human antibody. In some embodiments, the anti-LILRB1 antibody is an antibody fragment, e.g., a scFv. In some embodiments, the LILRB1 binding protein is a fusion protein comprising the anti-LILRB1 antibody provided herein. In other embodiments, the LILRB1 binding protein is a multispecific antibody comprising the anti-LILRB1 antibody provided herein. Other exemplary LILRB1 binding molecules are described in more detail in the following sections.

In some embodiments, the anti-LILRB1 antibody or antigen binding protein according to any of the above embodiments may incorporate any of the features, singly or in combination, as described in Sections 5.2.4 to 5.2.7 below.

In some embodiments, provided herein are antibodies that specifically bind to LILRB1 and can modulate LILRB1 activity and/or expression (e.g., inhibit LILRB1 mediated signaling). LILRB1 activity or LILRB1 signaling activity includes, but is not limited to, suppression of myeloid cells, suppression of myeloid cell activity, suppression of tumor-associated myeloid cells, suppression of NK cells, and suppression of cytolytic T-cells. In certain embodiments, a LILRB1 antagonist is provided herein that is an antibody described herein that specifically binds to LILRB1 and inhibits (including partially inhibits) at least one LILRB1 activity. In some embodiments, an anti-LILRB1 antibody inhibits LILRB1 signaling thereby reversing an LILRB1-induced suppressive effect. In some embodiments, the antibodies provided herein inhibit (including partially inhibit or reduce) the binding of LILRB1 to its ligand. In some embodiments, the ligands are MHC I molecules. In some embodiments, the ligand is HLA-A2. In some embodiments, the ligand is HLA-G. A LILRB1 activity can relate to any activity of LILRB1 such as those known or described in the art. In certain embodiments, LILRB1 activity and LILRB1 signaling (or LILRB1 mediated signaling) are used interchangeably herein.

In some embodiments, the antibody provided herein inhibits the interaction between LILRB1 and ligands of LILRB1 by 10%-99%. In other embodiments, the antibody provided herein inhibits the interaction between LILRB1 and ligands of LILRB1 100% (i.e., completely abolish the interaction as measured by an assay). In some embodiments, the antibody provided herein inhibits the interaction between LILRB1 and ligands of LILRB1 by at least 10%. In some embodiments, the antibody provided herein inhibits the interaction between LILRB1 and ligands of LILRB1 by at least 20%. In some embodiments, the antibody provided herein inhibits the interaction between LILRB1 and ligands of LILRB1 by at least 30%. In some embodiments, the antibody provided herein inhibits the interaction between LILRB1 and ligands of LILRB1 by at least 40%. In some embodiments, the antibody provided herein inhibits the interaction between LILRB1 and ligands of LILRB1 by at least 50%. In some embodiments, the antibody provided herein inhibits the interaction between LILRB1 and ligands of LILRB1 by at least 60%. In some embodiments, the antibody provided herein inhibits the interaction between LILRB1 and ligands of LILRB1 by at least 70%. In some embodiments, the antibody provided herein inhibits the interaction between LILRB1 and ligands of LILRB1 by at least 80%. In some embodiments, the antibody provided herein inhibits the interaction between LILRB1 and ligands of LILRB1 by at least 90%. In some embodiments, the antibody provided herein inhibits the interaction between LILRB1 and ligands of LILRB1 by about 10%-90%. In some embodiments, the antibody provided herein inhibits the interaction between LILRB1 and ligands of LILRB1 by about 20%-80%. In some embodiments, the antibody provided herein inhibits the interaction between LILRB1 and ligands of LILRB1 by about 30%-70%. In some embodiments, the antibody provided herein inhibits the interaction between LILRB1 and ligands of LILRB1 by about 40%-60%. In some embodiments, the LILRB1 and the ligands of LILRB1 are expressed on different cells. In some embodiments, the LILRB1 cell is expressed on a first cell, such as an immune cell. In some embodiments, the immune cell is an NK cell. In some embodiments, the immune cell is a macrophage. In some embodiments, the immune cell is a T cell. In some embodiments, the T cell is a cytotoxic T cell such as a CD8+ T cell. In some embodiments, the immune cell is a dendritic cell. In some embodiments, the ligands are MHC I molecules. In some embodiments, the ligand is HLA-A2. In some embodiments, the ligand is HLA-G.

In certain embodiments, the antibody described herein attenuates (e.g., partially attenuates) a LILRB1 activity. In some embodiments, the antibody provided herein attenuates a LILRB1 activity by at least about 10%. In some embodiments, the antibody provided herein attenuates a LILRB1 activity by at least about 20%. In some embodiments, the antibody provided herein attenuates a LILRB1 activity by at least about 30%. In some embodiments, the antibody provided herein attenuates a LILRB1 activity by at least about 40%. In some embodiments, the antibody provided herein attenuates a LILRB1 activity by at least about 50%. In some embodiments, the antibody provided herein attenuates a LILRB1 activity by at least about 60%. In some embodiments, the antibody provided herein attenuates a LILRB1 activity by at least about 70%. In some embodiments, the antibody provided herein attenuates a LILRB1 activity by at least about 80%. In some embodiments, the antibody provided herein attenuates a LILRB1 activity by at least about 90%. In some embodiments, the antibody provided herein attenuates a LILRB1 activity by at least about 95%. In certain embodiments, the antibody described herein can attenuate (e.g., partially attenuate) a LILRB1 activity by at least about 15% to about 65%. In certain embodiments, the antibody described herein can attenuate (e.g., partially attenuate) a LILRB1 activity by at least about 20% to about 65%. In certain embodiments, the antibody described herein can attenuate (e.g., partially attenuate) a LILRB1 activity by at least about 30% to about 65%.

In specific embodiments, the attenuation of a LILRB1 activity is assessed by methods described herein. In specific embodiments, the attenuation of a LILRB1 activity is assessed by methods known to one of skill in the art. In certain embodiments, the attenuation of a LILRB1 activity is relative to the LILRB1 activity in the presence of stimulation without any anti-LILRB1 antibody. In certain embodiments, the attenuation of a LILRB1 activity is relative to the LILRB1 activity in the presence of stimulation with an unrelated antibody (e.g., an antibody that does not specifically bind to LILRB1).

A non-limiting example of a LILRB1 activity is LILRB1 mediated signaling. Thus, in certain embodiments, the antibody described herein attenuates (e.g., partially attenuates) LILRB1 mediated signaling. In some embodiments, the antibody provided herein attenuates LILRB1 mediated signaling by at least about 10%. In some embodiments, the antibody provided herein attenuates LILRB1 mediated signaling by at least about 20%. In some embodiments, the antibody provided herein attenuates LILRB1 mediated signaling by at least about 30%. In some embodiments, the antibody provided herein attenuates LILRB1 mediated signaling by at least about 40%. In some embodiments, the antibody provided herein attenuates LILRB1 mediated signaling by at least about 50%. In some embodiments, the antibody provided herein attenuates LILRB1 mediated signaling by at least about 60%. In some embodiments, the antibody provided herein attenuates LILRB1 mediated signaling by at least about 70%. In some embodiments, the antibody provided herein attenuates LILRB1 mediated signaling by at least about 80%. In some embodiments, the antibody provided herein attenuates LILRB1 mediated signaling by at least about 90%. In some embodiments, the antibody provided herein attenuates LILRB1 mediated signaling by at least about 95%. In certain embodiments, the antibody described herein can attenuate (e.g., partially attenuate) LILRB1 mediated signaling by at least about 15% to about 65%. In certain embodiments, the antibody described herein can attenuate (e.g., partially attenuate) LILRB1 mediated signaling by at least about 20% to about 65%. In certain embodiments, the antibody described herein can attenuate (e.g., partially attenuate) LILRB1 mediated signaling by at least about 30% to about 65%.

In other embodiments, the antibody described herein can prevent suppression of an immune cell, e.g., a suppression mediated by the interaction between LILRB1 expressed on the immune cell with ligands of LILRB1 (e.g., expressed on a cancer or tumor cell). In some embodiments, the ligands are MHC I molecules. In some embodiments, the ligand is HLA-A2. In some embodiments, the ligand is HLA-G. In yet other embodiments, the antibody described herein can activate a response mediated by an immune cell, e.g., an anti-tumor response. In some embodiments, the immune cell is an NK cell. In some embodiments, the immune cell is a T cell. In some embodiments, the T cell is a cytotoxic T cell such as a CD8+ T cell. In some embodiments, the immune cell is a monocyte. In some embodiments, the immune cell is a macrophage. In some embodiments, the immune cell is a dendritic cell. In some embodiments, the immune cell mediates an anti-cancer/tumor response. In further embodiments, the cancer or tumor cell expresses HLA-A2. Additionally or alternatively, the cancer or tumor cell expresses HLA-G.

In some embodiments, the antibody described herein can increases myeloid cell activity. In some embodiments, the antibody described herein can enhance the activation of NK cells. In some embodiments, the antibody described herein can enhance macrophage activity. In some embodiments, the antibody described herein can enhance macrophage phagocytosis. In some embodiments, the antibody described herein can enhance T cell activity. In some embodiments, the antibody described herein can enhance the activity of CD8+ T cells. In some embodiments, the antibody described herein can enhance dendritic cell activity. In some embodiments, the antibody described herein can enhance FcR-mediated dendritic cell activity. In some embodiments, the antibody described herein can enhance cytokine release by immune cells. In some embodiments, the antibody described herein can enhance TNFα release by immune cells. In some embodiments, the antibody described herein can enhance IFN-γ release by immune cells.

In some embodiments, the antibody described herein enhances anti-tumor immunity by at least 10%. In some embodiments, the antibody described herein enhances anti-tumor immunity by at least 20%. In some embodiments, the antibody described herein enhances anti-tumor immunity by at least 30%. In some embodiments, the antibody described herein enhances anti-tumor immunity by at least 40%. In some embodiments, the antibody described herein enhances anti-tumor immunity by at least 50%. In some embodiments, the antibody described herein enhances anti-tumor immunity by at least 60%. In some embodiments, the antibody described herein enhances anti-tumor immunity by at least 70%. In some embodiments, the antibody described herein enhances anti-tumor immunity by at least 80%. In some embodiments, the antibody described herein enhances anti-tumor immunity by at least 90%. In some embodiments, the antibody described herein enhances anti-tumor immunity by at least 95%. In certain embodiments, the antibody described herein enhances anti-tumor immunity by at least about 15% to about 65%. In certain embodiments, the antibody described herein enhances anti-tumor immunity by at least about 20% to about 65%. In certain embodiments, the antibody described herein enhances anti-tumor immunity by at least about 30% to about 65%.

5.2.2. Antibodies that Bind to LILRB2

In one aspect, provided herein are binding agents (e.g., antibodies including fragments thereof) capable of binding to LILRB2. In some embodiments, the antibodies provided herein bind to human LILRB2. In some embodiments, the antibodies provided herein bind to rhesus macaque LILRB2. In some embodiments, the antibodies provided herein bind to cynomolgus LILRB2.

As used herein, LILRB2 refers to a LILRB2 polypeptide, a LILRB2 polypeptide fragment, a LILRB2 peptide or a LILRB2 epitope. In some embodiments, the LILRB2 binding agents are derived from human or humanized antibodies (e.g., comprising human framework regions) that bind LILRB2, including a LILRB2 polypeptide, a LILRB2 polypeptide fragment, a LILRB2 peptide or a LILRB2 epitope. In some embodiments, the binding agent (e.g., an antibody including fragment thereof) can bind to LILRB2 expressed on the surface of a mammalian (e.g., human) cell, including a LILRB2 expressing immune cell (e.g., an NK cell or a T cell). In some embodiments, the binding agent (e.g., an antibody including fragment thereof) provided herein binds a LILRB2 extracellular epitope exposed on a cell such as an immune cell. In some embodiments, described herein is a binding agent (e.g., an antibody including fragment thereof) that binds to LILRB2, such as human LILRB2 or a portion thereof. In some embodiments, LILRB2 is a human LILRB2. In some embodiments, the binding agent provided herein is a human LILRB2 binding agent (e.g., an antibody that binds to human LILRB2).

In some embodiments, the anti-LILRB2 antibody provided herein is an antagonist antibody. In one embodiment, the antibodies according to the disclosure are LILRB2 antagonists with no or low agonistic activity. In another embodiment, the antibody or functional fragment comprising an antigen-binding portion binds the target protein LILRB2 and decreases the binding of ligands of LILRB2 to LILRB2 to a basal level. In one aspect of this embodiment, the antibody or functional fragment reduces the amount of ligands that bind to LILRB2. In a further aspect of this embodiment, the antibody or functional fragment completely prevents ligands of LILRB2 from binding to LILRB2. In some embodiments, the ligands are MHC I molecules. In some embodiments, the ligand is HLA-A2. In some embodiments, the ligand is HLA-G. In some embodiments, the ligand is ANGPTLs. In some embodiments, the antibody or functional fragment provided herein inhibits one or more of these LILRB2 functional properties. An antibody that inhibits one or more of these LILRB2 functional properties (e.g. biochemical, immunochemical, cellular, physiological or other biological activities, or the like) as determined according to methodologies known to the art and described herein, will be understood to relate to a statistically significant decrease in the particular activity relative to that seen in the absence of the antibody (e.g. or when a control antibody of irrelevant specificity is present). In some embodiments, an antibody that inhibits LILRB2 activity effects such a statistically significant decrease by at least 10% of the measured parameter, by at least 50%, 80% or 90%, and in certain embodiments an antibody of the disclosure may inhibit greater than 95%, 98% or 99% of LILRB2 functional activity.

In some embodiments, the anti-LILRB2 antibody provided herein binds to LILRB2 (e.g., human LILRB2) with a dissociation constant (K_{D}) of ≤1 μM, ≤100 nM, ≤10 nM, ≤1 nM, ≤0.1 nM, ≤0.01 nM, or ≤0.001 nM (e.g. 10⁻⁸M or less, e.g. from 10⁻⁸ M to 10⁻¹³ M, e.g., from 10⁻⁹ M to 10⁻¹³ M). A variety of methods of measuring binding affinity are known in the art, any of which can be used for purposes of the present disclosure, including by RIA, for example, performed with the Fab version of an antibody of interest and its antigen (Chen et al., 1999, J. Mol Biol 293:865-81); by biolayer interferometry (BLI) or surface plasmon resonance (SPR) assays by Octet®, using, for example, an Octet®Red96 system, or by Biacore®, using, for example, a Biacore®TM-2000 or a Biacore®TM-3000. An “on-rate” or “rate of association” or “association rate” or “kon” may also be determined with the same biolayer interferometry (BLI) or surface plasmon resonance (SPR) techniques described above using, for example, the OCTET®Red96, the BIACORE®TM-2000, the BIACORE®TM-3000 system, the BIACORE®TM-8K, or the BIACORE®TM-8K+system.

In one embodiment, the anti-LILRB2 antibodies of the disclosure do not cross-react with one or more LILRB or LILRA family members (e.g. LILRB1, LILRA1, LILRA2, etc.) . . . . In one embodiment, the anti-LILRB2 antibodies of the disclosure cross-react with one or more LILRB and LILRA family members (e.g. LILRB1, LILRA1, LILRA2, etc.).

In some embodiments, the anti-LILRB2 antibodies provide herein are those described in Section 7 below. Thus, in some embodiments, the antibody provided herein comprises one or more CDR sequences of any one of SEQ ID NOs: 401-494, and 1167-1178. CDR sequences can be determined according to well-known numbering systems. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the anti-LILRB2 antibody is humanized. In some embodiments, the anti-LILRB2 antibody comprises an acceptor human framework, e.g., a human immunoglobulin framework or a human consensus framework.

In some embodiments, the anti-LILRB2 antibody provided herein comprises a HCDR1, a HCDR2, and a HCDR3 as set forth in a VH comprising the amino acid sequence of SEQ ID NO: 401. In some embodiments, the anti-LILRB2 antibody provided herein comprises a HCDR1, a HCDR2, and a HCDR3 as set forth in a VH comprising the amino acid sequence of SEQ ID NO: 403. In some embodiments, the anti-LILRB2 antibody provided herein comprises a HCDR1, a HCDR2, and a HCDR3 as set forth in a VH comprising the amino acid sequence of SEQ ID NO: 405. In some embodiments, the anti-LILRB2 antibody provided herein comprises a HCDR1, a HCDR2, and a HCDR3 as set forth in a VH comprising the amino acid sequence of SEQ ID NO: 407. In some embodiments, the anti-LILRB2 antibody provided herein comprises a HCDR1, a HCDR2, and a HCDR3 as set forth in a VH comprising the amino acid sequence of SEQ ID NO: 409. In some embodiments, the anti-LILRB2 antibody provided herein comprises a HCDR1, a HCDR2, and a HCDR3 as set forth in a VH comprising the amino acid sequence of SEQ ID NO: 411. In some embodiments, the anti-LILRB2 antibody provided herein comprises a HCDR1, a HCDR2, and a HCDR3 as set forth in a VH comprising the amino acid sequence of SEQ ID NO: 413. In some embodiments, the anti-LILRB2 antibody provided herein comprises a HCDR1, a HCDR2, and a HCDR3 as set forth in a VH comprising the amino acid sequence of SEQ ID NO: 415. In some embodiments, the anti-LILRB2 antibody provided herein comprises a HCDR1, a HCDR2, and a HCDR3 as set forth in a VH comprising the amino acid sequence of SEQ ID NO: 417. In some embodiments, the anti-LILRB2 antibody provided herein comprises a HCDR1, a HCDR2, and a HCDR3 as set forth in a VH comprising the amino acid sequence of SEQ ID NO: 419. In some embodiments, the anti-LILRB2 antibody provided herein comprises a HCDR1, a HCDR2, and a HCDR3 as set forth in a VH comprising the amino acid sequence of SEQ ID NO: 421. In some embodiments, the anti-LILRB2 antibody provided herein comprises a HCDR1, a HCDR2, and a HCDR3 as set forth in a VH comprising the amino acid sequence of SEQ ID NO: 423. In some embodiments, the anti-LILRB2 antibody provided herein comprises a HCDR1, a HCDR2, and a HCDR3 as set forth in a VH comprising the amino acid sequence of SEQ ID NO: 425. In some embodiments, the anti-LILRB2 antibody provided herein comprises a HCDR1, a HCDR2, and a HCDR3 as set forth in a VH comprising the amino acid sequence of SEQ ID NO: 427. In some embodiments, the anti-LILRB2 antibody provided herein comprises a HCDR1, a HCDR2, and a HCDR3 as set forth in a VH comprising the amino acid sequence of SEQ ID NO: 429. In some embodiments, the anti-LILRB2 antibody provided herein comprises a HCDR1, a HCDR2, and a HCDR3 as set forth in a VH comprising the amino acid sequence of SEQ ID NO: 431. In some embodiments, the anti-LILRB2 antibody provided herein comprises a HCDR1, a HCDR2, and a HCDR3 as set forth in a VH comprising the amino acid sequence of SEQ ID NO: 433. In some embodiments, the anti-LILRB2 antibody provided herein comprises a HCDR1, a HCDR2, and a HCDR3 as set forth in a VH comprising the amino acid sequence of SEQ ID NO: 435. In some embodiments, the anti-LILRB2 antibody provided herein comprises a HCDR1, a HCDR2, and a HCDR3 as set forth in a VH comprising the amino acid sequence of SEQ ID NO: 437. In some embodiments, the anti-LILRB2 antibody provided herein comprises a HCDR1, a HCDR2, and a HCDR3 as set forth in a VH comprising the amino acid sequence of SEQ ID NO: 439. In some embodiments, the anti-LILRB2 antibody provided herein comprises a HCDR1, a HCDR2, and a HCDR3 as set forth in a VH comprising the amino acid sequence of SEQ ID NO: 441. In some embodiments, the anti-LILRB2 antibody provided herein comprises a HCDR1, a HCDR2, and a HCDR3 as set forth in a VH comprising the amino acid sequence of SEQ ID NO: 443. In some embodiments, the anti-LILRB2 antibody provided herein comprises a HCDR1, a HCDR2, and a HCDR3 as set forth in a VH comprising the amino acid sequence of SEQ ID NO: 445. In some embodiments, the anti-LILRB2 antibody provided herein comprises a HCDR1, a HCDR2, and a HCDR3 as set forth in a VH comprising the amino acid sequence of SEQ ID NO: 447. In some embodiments, the anti-LILRB2 antibody provided herein comprises a HCDR1, a HCDR2, and a HCDR3 as set forth in a VH comprising the amino acid sequence of SEQ ID NO: 449. In some embodiments, the anti-LILRB2 antibody provided herein comprises a HCDR1, a HCDR2, and a HCDR3 as set forth in a VH comprising the amino acid sequence of SEQ ID NO: 451. In some embodiments, the anti-LILRB2 antibody provided herein comprises a HCDR1, a HCDR2, and a HCDR3 as set forth in a VH comprising the amino acid sequence of SEQ ID NO: 453. In some embodiments, the anti-LILRB2 antibody provided herein comprises a HCDR1, a HCDR2, and a HCDR3 as set forth in a VH comprising the amino acid sequence of SEQ ID NO: 455. In some embodiments, the anti-LILRB2 antibody provided herein comprises a HCDR1, a HCDR2, and a HCDR3 as set forth in a VH comprising the amino acid sequence of SEQ ID NO: 457. In some embodiments, the anti-LILRB2 antibody provided herein comprises a HCDR1, a HCDR2, and a HCDR3 as set forth in a VH comprising the amino acid sequence of SEQ ID NO: 459. In some embodiments, the anti-LILRB2 antibody provided herein comprises a HCDR1, a HCDR2, and a HCDR3 as set forth in a VH comprising the amino acid sequence of SEQ ID NO: 461. In some embodiments, the anti-LILRB2 antibody provided herein comprises a HCDR1, a HCDR2, and a HCDR3 as set forth in a VH comprising the amino acid sequence of SEQ ID NO: 463. In some embodiments, the anti-LILRB2 antibody provided herein comprises a HCDR1, a HCDR2, and a HCDR3 as set forth in a VH comprising the amino acid sequence of SEQ ID NO: 465. In some embodiments, the anti-LILRB2 antibody provided herein comprises a HCDR1, a HCDR2, and a HCDR3 as set forth in a VH comprising the amino acid sequence of SEQ ID NO: 467. In some embodiments, the anti-LILRB2 antibody provided herein comprises a HCDR1, a HCDR2, and a HCDR3 as set forth in a VH comprising the amino acid sequence of SEQ ID NO: 469. In some embodiments, the anti-LILRB2 antibody provided herein comprises a HCDR1, a HCDR2, and a HCDR3 as set forth in a VH comprising the amino acid sequence of SEQ ID NO: 471. In some embodiments, the anti-LILRB2 antibody provided herein comprises a HCDR1, a HCDR2, and a HCDR3 as set forth in a VH comprising the amino acid sequence of SEQ ID NO: 473. In some embodiments, the anti-LILRB2 antibody provided herein comprises a HCDR1, a HCDR2, and a HCDR3 as set forth in a VH comprising the amino acid sequence of SEQ ID NO: 475. In some embodiments, the anti-LILRB2 antibody provided herein comprises a HCDR1, a HCDR2, and a HCDR3 as set forth in a VH comprising the amino acid sequence of SEQ ID NO: 477. In some embodiments, the anti-LILRB2 antibody provided herein comprises a HCDR1, a HCDR2, and a HCDR3 as set forth in a VH comprising the amino acid sequence of SEQ ID NO: 479. In some embodiments, the anti-LILRB2 antibody provided herein comprises a HCDR1, a HCDR2, and a HCDR3 as set forth in a VH comprising the amino acid sequence of SEQ ID NO: 481. In some embodiments, the anti-LILRB2 antibody provided herein comprises a HCDR1, a HCDR2, and a HCDR3 as set forth in a VH comprising the amino acid sequence of SEQ ID NO: 483. In some embodiments, the anti-LILRB2 antibody provided herein comprises a HCDR1, a HCDR2, and a HCDR3 as set forth in a VH comprising the amino acid sequence of SEQ ID NO: 485. In some embodiments, the anti-LILRB2 antibody provided herein comprises a HCDR1, a HCDR2, and a HCDR3 as set forth in a VH comprising the amino acid sequence of SEQ ID NO: 487. In some embodiments, the anti-LILRB2 antibody provided herein comprises a HCDR1, a HCDR2, and a HCDR3 as set forth in a VH comprising the amino acid sequence of SEQ ID NO: 489. In some embodiments, the anti-LILRB2 antibody provided herein comprises a HCDR1, a HCDR2, and a HCDR3 as set forth in a VH comprising the amino acid sequence of SEQ ID NO: 491. In some embodiments, the anti-LILRB2 antibody provided herein comprises a HCDR1, a HCDR2, and a HCDR3 as set forth in a VH comprising the amino acid sequence of SEQ ID NO: 493. In some embodiments, the anti-LILRB2 antibody provided herein comprises a HCDR1, a HCDR2, and a HCDR3 as set forth in a VH comprising the amino acid sequence of SEQ ID NO: 1167. In some embodiments, the anti-LILRB2 antibody provided herein comprises a HCDR1, a HCDR2, and a HCDR3 as set forth in a VH comprising the amino acid sequence of SEQ ID NO: 1169. In some embodiments, the anti-LILRB2 antibody provided herein comprises a HCDR1, a HCDR2, and a HCDR3 as set forth in a VH comprising the amino acid sequence of SEQ ID NO: 1171. In some embodiments, the anti-LILRB2 antibody provided herein comprises a HCDR1, a HCDR2, and a HCDR3 as set forth in a VH comprising the amino acid sequence of SEQ ID NO: 1173. In some embodiments, the anti-LILRB2 antibody provided herein comprises a HCDR1, a HCDR2, and a HCDR3 as set forth in a VH comprising the amino acid sequence of SEQ ID NO: 1175. In some embodiments, the anti-LILRB2 antibody provided herein comprises a HCDR1, a HCDR2, and a HCDR3 as set forth in a VH comprising the amino acid sequence of SEQ ID NO: 1177. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering.

In some embodiments, the anti-LILRB2 antibody provided herein comprises a LCDR1, a LCDR2, and a LCDR3 as set forth in a VL comprising the amino acid sequence of SEQ ID NO: 402. In some embodiments, the anti-LILRB2 antibody provided herein comprises a LCDR1, a LCDR2, and a LCDR3 as set forth in a VL comprising the amino acid sequence of SEQ ID NO: 404. In some embodiments, the anti-LILRB2 antibody provided herein comprises a LCDR1, a LCDR2, and a LCDR3 as set forth in a VL comprising the amino acid sequence of SEQ ID NO: 406. In some embodiments, the anti-LILRB2 antibody provided herein comprises a LCDR1, a LCDR2, and a LCDR3 as set forth in a VL comprising the amino acid sequence of SEQ ID NO: 408. In some embodiments, the anti-LILRB2 antibody provided herein comprises a LCDR1, a LCDR2, and a LCDR3 as set forth in a VL comprising the amino acid sequence of SEQ ID NO: 410. In some embodiments, the anti-LILRB2 antibody provided herein comprises a LCDR1, a LCDR2, and a LCDR3 as set forth in a VL comprising the amino acid sequence of SEQ ID NO: 412. In some embodiments, the anti-LILRB2 antibody provided herein comprises a LCDR1, a LCDR2, and a LCDR3 as set forth in a VL comprising the amino acid sequence of SEQ ID NO: 414. In some embodiments, the anti-LILRB2 antibody provided herein comprises a LCDR1, a LCDR2, and a LCDR3 as set forth in a VL comprising the amino acid sequence of SEQ ID NO: 416. In some embodiments, the anti-LILRB2 antibody provided herein comprises a LCDR1, a LCDR2, and a LCDR3 as set forth in a VL comprising the amino acid sequence of SEQ ID NO: 418. In some embodiments, the anti-LILRB2 antibody provided herein comprises a LCDR1, a LCDR2, and a LCDR3 as set forth in a VL comprising the amino acid sequence of SEQ ID NO: 420. In some embodiments, the anti-LILRB2 antibody provided herein comprises a LCDR1, a LCDR2, and a LCDR3 as set forth in a VL comprising the amino acid sequence of SEQ ID NO: 422. In some embodiments, the anti-LILRB2 antibody provided herein comprises a LCDR1, a LCDR2, and a LCDR3 as set forth in a VL comprising the amino acid sequence of SEQ ID NO: 424. In some embodiments, the anti-LILRB2 antibody provided herein comprises a LCDR1, a LCDR2, and a LCDR3 as set forth in a VL comprising the amino acid sequence of SEQ ID NO: 426. In some embodiments, the anti-LILRB2 antibody provided herein comprises a LCDR1, a LCDR2, and a LCDR3 as set forth in a VL comprising the amino acid sequence of SEQ ID NO: 428. In some embodiments, the anti-LILRB2 antibody provided herein comprises a LCDR1, a LCDR2, and a LCDR3 as set forth in a VL comprising the amino acid sequence of SEQ ID NO: 430. In some embodiments, the anti-LILRB2 antibody provided herein comprises a LCDR1, a LCDR2, and a LCDR3 as set forth in a VL comprising the amino acid sequence of SEQ ID NO: 432. In some embodiments, the anti-LILRB2 antibody provided herein comprises a LCDR1, a LCDR2, and a LCDR3 as set forth in a VL comprising the amino acid sequence of SEQ ID NO: 434. In some embodiments, the anti-LILRB2 antibody provided herein comprises a LCDR1, a LCDR2, and a LCDR3 as set forth in a VL comprising the amino acid sequence of SEQ ID NO: 436. In some embodiments, the anti-LILRB2 antibody provided herein comprises a LCDR1, a LCDR2, and a LCDR3 as set forth in a VL comprising the amino acid sequence of SEQ ID NO: 438. In some embodiments, the anti-LILRB2 antibody provided herein comprises a LCDR1, a LCDR2, and a LCDR3 as set forth in a VL comprising the amino acid sequence of SEQ ID NO: 440. In some embodiments, the anti-LILRB2 antibody provided herein comprises a LCDR1, a LCDR2, and a LCDR3 as set forth in a VL comprising the amino acid sequence of SEQ ID NO: 442. In some embodiments, the anti-LILRB2 antibody provided herein comprises a LCDR1, a LCDR2, and a LCDR3 as set forth in a VL comprising the amino acid sequence of SEQ ID NO: 444. In some embodiments, the anti-LILRB2 antibody provided herein comprises a LCDR1, a LCDR2, and a LCDR3 as set forth in a VL comprising the amino acid sequence of SEQ ID NO: 446. In some embodiments, the anti-LILRB2 antibody provided herein comprises a LCDR1, a LCDR2, and a LCDR3 as set forth in a VL comprising the amino acid sequence of SEQ ID NO: 448. In some embodiments, the anti-LILRB2 antibody provided herein comprises a LCDR1, a LCDR2, and a LCDR3 as set forth in a VL comprising the amino acid sequence of SEQ ID NO: 450. In some embodiments, the anti-LILRB2 antibody provided herein comprises a LCDR1, a LCDR2, and a LCDR3 as set forth in a VL comprising the amino acid sequence of SEQ ID NO: 452. In some embodiments, the anti-LILRB2 antibody provided herein comprises a LCDR1, a LCDR2, and a LCDR3 as set forth in a VL comprising the amino acid sequence of SEQ ID NO: 454. In some embodiments, the anti-LILRB2 antibody provided herein comprises a LCDR1, a LCDR2, and a LCDR3 as set forth in a VL comprising the amino acid sequence of SEQ ID NO: 456. In some embodiments, the anti-LILRB2 antibody provided herein comprises a LCDR1, a LCDR2, and a LCDR3 as set forth in a VL comprising the amino acid sequence of SEQ ID NO: 458. In some embodiments, the anti-LILRB2 antibody provided herein comprises a LCDR1, a LCDR2, and a LCDR3 as set forth in a VL comprising the amino acid sequence of SEQ ID NO: 460. In some embodiments, the anti-LILRB2 antibody provided herein comprises a LCDR1, a LCDR2, and a LCDR3 as set forth in a VL comprising the amino acid sequence of SEQ ID NO: 462. In some embodiments, the anti-LILRB2 antibody provided herein comprises a LCDR1, a LCDR2, and a LCDR3 as set forth in a VL comprising the amino acid sequence of SEQ ID NO: 464. In some embodiments, the anti-LILRB2 antibody provided herein comprises a LCDR1, a LCDR2, and a LCDR3 as set forth in a VL comprising the amino acid sequence of SEQ ID NO: 466. In some embodiments, the anti-LILRB2 antibody provided herein comprises a LCDR1, a LCDR2, and a LCDR3 as set forth in a VL comprising the amino acid sequence of SEQ ID NO: 468. In some embodiments, the anti-LILRB2 antibody provided herein comprises a LCDR1, a LCDR2, and a LCDR3 as set forth in a VL comprising the amino acid sequence of SEQ ID NO: 470. In some embodiments, the anti-LILRB2 antibody provided herein comprises a LCDR1, a LCDR2, and a LCDR3 as set forth in a VL comprising the amino acid sequence of SEQ ID NO: 472. In some embodiments, the anti-LILRB2 antibody provided herein comprises a LCDR1, a LCDR2, and a LCDR3 as set forth in a VL comprising the amino acid sequence of SEQ ID NO: 474. In some embodiments, the anti-LILRB2 antibody provided herein comprises a LCDR1, a LCDR2, and a LCDR3 as set forth in a VL comprising the amino acid sequence of SEQ ID NO: 476. In some embodiments, the anti-LILRB2 antibody provided herein comprises a LCDR1, a LCDR2, and a LCDR3 as set forth in a VL comprising the amino acid sequence of SEQ ID NO: 478. In some embodiments, the anti-LILRB2 antibody provided herein comprises a LCDR1, a LCDR2, and a LCDR3 as set forth in a VL comprising the amino acid sequence of SEQ ID NO: 480. In some embodiments, the anti-LILRB2 antibody provided herein comprises a LCDR1, a LCDR2, and a LCDR3 as set forth in a VL comprising the amino acid sequence of SEQ ID NO: 482. In some embodiments, the anti-LILRB2 antibody provided herein comprises a LCDR1, a LCDR2, and a LCDR3 as set forth in a VL comprising the amino acid sequence of SEQ ID NO: 484. In some embodiments, the anti-LILRB2 antibody provided herein comprises a LCDR1, a LCDR2, and a LCDR3 as set forth in a VL comprising the amino acid sequence of SEQ ID NO: 486. In some embodiments, the anti-LILRB2 antibody provided herein comprises a LCDR1, a LCDR2, and a LCDR3 as set forth in a VL comprising the amino acid sequence of SEQ ID NO: 488. In some embodiments, the anti-LILRB2 antibody provided herein comprises a LCDR1, a LCDR2, and a LCDR3 as set forth in a VL comprising the amino acid sequence of SEQ ID NO: 490. In some embodiments, the anti-LILRB2 antibody provided herein comprises a LCDR1, a LCDR2, and a LCDR3 as set forth in a VL comprising the amino acid sequence of SEQ ID NO: 492. In some embodiments, the anti-LILRB2 antibody provided herein comprises a LCDR1, a LCDR2, and a LCDR3 as set forth in a VL comprising the amino acid sequence of SEQ ID NO: 494. In some embodiments, the anti-LILRB2 antibody provided herein comprises a LCDR1, a LCDR2, and a LCDR3 as set forth in a VL comprising the amino acid sequence of SEQ ID NO: 1168. In some embodiments, the anti-LILRB2 antibody provided herein comprises a LCDR1, a LCDR2, and a LCDR3 as set forth in a VL comprising the amino acid sequence of SEQ ID NO: 1170. In some embodiments, the anti-LILRB2 antibody provided herein comprises a LCDR1, a LCDR2, and a LCDR3 as set forth in a VL comprising the amino acid sequence of SEQ ID NO: 1172. In some embodiments, the anti-LILRB2 antibody provided herein comprises a LCDR1, a LCDR2, and a LCDR3 as set forth in a VL comprising the amino acid sequence of SEQ ID NO: 1174. In some embodiments, the anti-LILRB2 antibody provided herein comprises a LCDR1, a LCDR2, and a LCDR3 as set forth in a VL comprising the amino acid sequence of SEQ ID NO: 1176. In some embodiments, the anti-LILRB2 antibody provided herein comprises a LCDR1, a LCDR2, and a LCDR3 as set forth in a VL comprising the amino acid sequence of SEQ ID NO: 1178. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering.

In some embodiments, the antibody or antigen binding fragment provided herein comprises a HCDR1, a HCDR2, and/or a HCDR3 as set forth in a VH comprising the amino acid sequence of SEQ ID NO: 401, and/or a LCDR1, a LCDR2, and/or a LCDR3 as set forth in a VL comprising the amino acid sequence of SEQ ID NO: 402. In some embodiments, the antibody or antigen binding fragment provided herein comprises a HCDR1, a HCDR2, and a HCDR3 as set forth in a VH comprising the amino acid sequence of SEQ ID NO: 401, and a LCDR1, a LCDR2, and a LCDR3 as set forth in a VL comprising the amino acid sequence of SEQ ID NO: 402.

In some embodiments, the antibody or antigen binding fragment provided herein comprises a HCDR1, a HCDR2, and/or a HCDR3 as set forth in a VH comprising the amino acid sequence of SEQ ID NO: 403, and/or a LCDR1, a LCDR2, and/or a LCDR3 as set forth in a VL comprising the amino acid sequence of SEQ ID NO: 404. In some embodiments, the antibody or antigen binding fragment provided herein comprises a HCDR1, a HCDR2, and a HCDR3 as set forth in a VH comprising the amino acid sequence of SEQ ID NO: 403, and a LCDR1, a LCDR2, and a LCDR3 as set forth in a VL comprising the amino acid sequence of SEQ ID NO: 404.

In some embodiments, the antibody or antigen binding fragment provided herein sequence of SEQ ID NO: 405, and/or a LCDR1, a LCDR2, and/or a LCDR3 as set forth in a VL comprising the amino acid sequence of SEQ ID NO: 406. In some embodiments, the antibody or antigen binding fragment provided herein comprises a HCDR1, a HCDR2, and a HCDR3 as set forth in a VH comprising the amino acid sequence of SEQ ID NO: 405, and a LCDR1, a LCDR2, and a LCDR3 as set forth in a VL comprising the amino acid sequence of SEQ ID NO: 406.

In some embodiments, the antibody or antigen binding fragment provided herein comprises a HCDR1, a HCDR2, and/or a HCDR3 as set forth in a VH comprising the amino acid sequence of SEQ ID NO: 407, and/or a LCDR1, a LCDR2, and/or a LCDR3 as set forth in a VL comprising the amino acid sequence of SEQ ID NO: 408. In some embodiments, the antibody or antigen binding fragment provided herein comprises a HCDR1, a HCDR2, and a HCDR3 as set forth in a VH comprising the amino acid sequence of SEQ ID NO: 407, and a LCDR1, a LCDR2, and a LCDR3 as set forth in a VL comprising the amino acid sequence of SEQ ID NO: 408.

In some embodiments, the antibody or antigen binding fragment provided herein comprises a HCDR1, a HCDR2, and/or a HCDR3 as set forth in a VH comprising the amino acid sequence of SEQ ID NO: 409, and/or a LCDR1, a LCDR2, and/or a LCDR3 as set forth in a VL comprising the amino acid sequence of SEQ ID NO: 410. In some embodiments, the antibody or antigen binding fragment provided herein comprises a HCDR1, a HCDR2, and a HCDR3 as set forth in a VH comprising the amino acid sequence of SEQ ID NO: 409, and a LCDR1, a LCDR2, and a LCDR3 as set forth in a VL comprising the amino acid sequence of SEQ ID NO: 410.

In some embodiments, the antibody or antigen binding fragment provided herein comprises a HCDR1, a HCDR2, and/or a HCDR3 as set forth in a VH comprising the amino acid sequence of SEQ ID NO: 411, and/or a LCDR1, a LCDR2, and/or a LCDR3 as set forth in a VL comprising the amino acid sequence of SEQ ID NO: 412. In some embodiments, the antibody or antigen binding fragment provided herein comprises a HCDR1, a HCDR2, and a HCDR3 as set forth in a VH comprising the amino acid sequence of SEQ ID NO: 411, and a LCDR1, a LCDR2, and a LCDR3 as set forth in a VL comprising the amino acid sequence of SEQ ID NO: 412.

In some embodiments, the antibody or antigen binding fragment provided herein comprises a HCDR1, a HCDR2, and/or a HCDR3 as set forth in a VH comprising the amino acid sequence of SEQ ID NO: 413, and/or a LCDR1, a LCDR2, and/or a LCDR3 as set forth in a VL comprising the amino acid sequence of SEQ ID NO: 414. In some embodiments, the antibody or antigen binding fragment provided herein comprises a HCDR1, a HCDR2, and a HCDR3 as set forth in a VH comprising the amino acid sequence of SEQ ID NO: 413, and a LCDR1, a LCDR2, and a LCDR3 as set forth in a VL comprising the amino acid sequence of SEQ ID NO: 414.

In some embodiments, the antibody or antigen binding fragment provided herein comprises a HCDR1, a HCDR2, and/or a HCDR3 as set forth in a VH comprising the amino acid sequence of SEQ ID NO: 415, and/or a LCDR1, a LCDR2, and/or a LCDR3 as set forth in a VL comprising the amino acid sequence of SEQ ID NO: 416. In some embodiments, the antibody or antigen binding fragment provided herein comprises a HCDR1, a HCDR2, and a HCDR3 as set forth in a VH comprising the amino acid sequence of SEQ ID NO: 415, and a LCDR1, a LCDR2, and a LCDR3 as set forth in a VL comprising the amino acid sequence of SEQ ID NO: 416.

In some embodiments, the antibody or antigen binding fragment provided herein comprises a HCDR1, a HCDR2, and/or a HCDR3 as set forth in a VH comprising the amino acid sequence of SEQ ID NO: 417, and/or a LCDR1, a LCDR2, and/or a LCDR3 as set forth in a VL comprising the amino acid sequence of SEQ ID NO: 418. In some embodiments, the antibody or antigen binding fragment provided herein comprises a HCDR1, a HCDR2, and a HCDR3 as set forth in a VH comprising the amino acid sequence of SEQ ID NO: 417, and a LCDR1, a LCDR2, and a LCDR3 as set forth in a VL comprising the amino acid sequence of SEQ ID NO: 418.

In some embodiments, the antibody or antigen binding fragment provided herein comprises a HCDR1, a HCDR2, and/or a HCDR3 as set forth in a VH comprising the amino acid sequence of SEQ ID NO: 419, and/or a LCDR1, a LCDR2, and/or a LCDR3 as set forth in a VL comprising the amino acid sequence of SEQ ID NO: 420. In some embodiments, the antibody or antigen binding fragment provided herein comprises a HCDR1, a HCDR2, and a HCDR3 as set forth in a VH comprising the amino acid sequence of SEQ ID NO: 419, and a LCDR1, a LCDR2, and a LCDR3 as set forth in a VL comprising the amino acid sequence of SEQ ID NO: 420.

In some embodiments, the antibody or antigen binding fragment provided herein comprises a HCDR1, a HCDR2, and/or a HCDR3 as set forth in a VH comprising the amino acid sequence of SEQ ID NO: 421, and/or a LCDR1, a LCDR2, and/or a LCDR3 as set forth in a VL comprising the amino acid sequence of SEQ ID NO: 422. In some embodiments, the antibody or antigen binding fragment provided herein comprises a HCDR1, a HCDR2, and a HCDR3 as set forth in a VH comprising the amino acid sequence of SEQ ID NO: 421, and a LCDR1, a LCDR2, and a LCDR3 as set forth in a VL comprising the amino acid sequence of SEQ ID NO: 422.

In some embodiments, the antibody or antigen binding fragment provided herein sequence of SEQ ID NO: 423, and/or a LCDR1, a LCDR2, and/or a LCDR3 as set forth in a VL comprising the amino acid sequence of SEQ ID NO: 424. In some embodiments, the antibody or antigen binding fragment provided herein comprises a HCDR1, a HCDR2, and a HCDR3 as set forth in a VH comprising the amino acid sequence of SEQ ID NO: 423, and a LCDR1, a LCDR2, and a LCDR3 as set forth in a VL comprising the amino acid sequence of SEQ ID NO: 424.

In some embodiments, the antibody or antigen binding fragment provided herein comprises a HCDR1, a HCDR2, and/or a HCDR3 as set forth in a VH comprising the amino acid sequence of SEQ ID NO: 425, and/or a LCDR1, a LCDR2, and/or a LCDR3 as set forth in a VL comprising the amino acid sequence of SEQ ID NO: 426. In some embodiments, the antibody or antigen binding fragment provided herein comprises a HCDR1, a HCDR2, and a HCDR3 as set forth in a VH comprising the amino acid sequence of SEQ ID NO: 425, and a LCDR1, a LCDR2, and a LCDR3 as set forth in a VL comprising the amino acid sequence of SEQ ID NO: 426.

In some embodiments, the antibody or antigen binding fragment provided herein comprises a HCDR1, a HCDR2, and/or a HCDR3 as set forth in a VH comprising the amino acid sequence of SEQ ID NO: 427, and/or a LCDR1, a LCDR2, and/or a LCDR3 as set forth in a VL comprising the amino acid sequence of SEQ ID NO: 428. In some embodiments, the antibody or antigen binding fragment provided herein comprises a HCDR1, a HCDR2, and a HCDR3 as set forth in a VH comprising the amino acid sequence of SEQ ID NO: 427, and a LCDR1, a LCDR2, and a LCDR3 as set forth in a VL comprising the amino acid sequence of SEQ ID NO: 428.

In some embodiments, the antibody or antigen binding fragment provided herein comprises a HCDR1, a HCDR2, and/or a HCDR3 as set forth in a VH comprising the amino acid sequence of SEQ ID NO: 429, and/or a LCDR1, a LCDR2, and/or a LCDR3 as set forth in a VL comprising the amino acid sequence of SEQ ID NO: 430. In some embodiments, the antibody or antigen binding fragment provided herein comprises a HCDR1, a HCDR2, and a HCDR3 as set forth in a VH comprising the amino acid sequence of SEQ ID NO: 429, and a LCDR1, a LCDR2, and a LCDR3 as set forth in a VL comprising the amino acid sequence of SEQ ID NO: 430.

In some embodiments, the antibody or antigen binding fragment provided herein comprises a HCDR1, a HCDR2, and/or a HCDR3 as set forth in a VH comprising the amino acid sequence of SEQ ID NO: 431, and/or a LCDR1, a LCDR2, and/or a LCDR3 as set forth in a VL comprising the amino acid sequence of SEQ ID NO: 432. In some embodiments, the antibody or antigen binding fragment provided herein comprises a HCDR1, a HCDR2, and a HCDR3 as set forth in a VH comprising the amino acid sequence of SEQ ID NO: 431, and a LCDR1, a LCDR2, and a LCDR3 as set forth in a VL comprising the amino acid sequence of SEQ ID NO: 432.

In some embodiments, the antibody or antigen binding fragment provided herein comprises a HCDR1, a HCDR2, and/or a HCDR3 as set forth in a VH comprising the amino acid sequence of SEQ ID NO: 433, and/or a LCDR1, a LCDR2, and/or a LCDR3 as set forth in a VL comprising the amino acid sequence of SEQ ID NO: 434. In some embodiments, the antibody or antigen binding fragment provided herein comprises a HCDR1, a HCDR2, and a HCDR3 as set forth in a VH comprising the amino acid sequence of SEQ ID NO: 433, and a LCDR1, a LCDR2, and a LCDR3 as set forth in a VL comprising the amino acid sequence of SEQ ID NO: 434.

In some embodiments, the antibody or antigen binding fragment provided herein comprises a HCDR1, a HCDR2, and/or a HCDR3 as set forth in a VH comprising the amino acid sequence of SEQ ID NO: 435, and/or a LCDR1, a LCDR2, and/or a LCDR3 as set forth in a VL comprising the amino acid sequence of SEQ ID NO: 436. In some embodiments, the antibody or antigen binding fragment provided herein comprises a HCDR1, a HCDR2, and a HCDR3 as set forth in a VH comprising the amino acid sequence of SEQ ID NO: 435, and a LCDR1, a LCDR2, and a LCDR3 as set forth in a VL comprising the amino acid sequence of SEQ ID NO: 436.

In some embodiments, the antibody or antigen binding fragment provided herein comprises a HCDR1, a HCDR2, and/or a HCDR3 as set forth in a VH comprising the amino acid sequence of SEQ ID NO: 437, and/or a LCDR1, a LCDR2, and/or a LCDR3 as set forth in a VL comprising the amino acid sequence of SEQ ID NO: 438. In some embodiments, the antibody or antigen binding fragment provided herein comprises a HCDR1, a HCDR2, and a HCDR3 as set forth in a VH comprising the amino acid sequence of SEQ ID NO: 437, and a LCDR1, a LCDR2, and a LCDR3 as set forth in a VL comprising the amino acid sequence of SEQ ID NO: 438.

In some embodiments, the antibody or antigen binding fragment provided herein comprises a HCDR1, a HCDR2, and/or a HCDR3 as set forth in a VH comprising the amino acid sequence of SEQ ID NO: 439, and/or a LCDR1, a LCDR2, and/or a LCDR3 as set forth in a VL comprising the amino acid sequence of SEQ ID NO: 440. In some embodiments, the antibody or antigen binding fragment provided herein comprises a HCDR1, a HCDR2, and a HCDR3 as set forth in a VH comprising the amino acid sequence of SEQ ID NO: 439, and a LCDR1, a LCDR2, and a LCDR3 as set forth in a VL comprising the amino acid sequence of SEQ ID NO: 440.

In some embodiments, the antibody or antigen binding fragment provided herein sequence of SEQ ID NO: 441, and/or a LCDR1, a LCDR2, and/or a LCDR3 as set forth in a VL comprising the amino acid sequence of SEQ ID NO: 442. In some embodiments, the antibody or antigen binding fragment provided herein comprises a HCDR1, a HCDR2, and a HCDR3 as set forth in a VH comprising the amino acid sequence of SEQ ID NO: 441, and a LCDR1, a LCDR2, and a LCDR3 as set forth in a VL comprising the amino acid sequence of SEQ ID NO: 442.

In some embodiments, the antibody or antigen binding fragment provided herein comprises a HCDR1, a HCDR2, and/or a HCDR3 as set forth in a VH comprising the amino acid sequence of SEQ ID NO: 443, and/or a LCDR1, a LCDR2, and/or a LCDR3 as set forth in a VL comprising the amino acid sequence of SEQ ID NO: 444. In some embodiments, the antibody or antigen binding fragment provided herein comprises a HCDR1, a HCDR2, and a HCDR3 as set forth in a VH comprising the amino acid sequence of SEQ ID NO: 443, and a LCDR1, a LCDR2, and a LCDR3 as set forth in a VL comprising the amino acid sequence of SEQ ID NO: 444.

In some embodiments, the antibody or antigen binding fragment provided herein comprises a HCDR1, a HCDR2, and/or a HCDR3 as set forth in a VH comprising the amino acid sequence of SEQ ID NO: 445, and/or a LCDR1, a LCDR2, and/or a LCDR3 as set forth in a VL comprising the amino acid sequence of SEQ ID NO: 446. In some embodiments, the antibody or antigen binding fragment provided herein comprises a HCDR1, a HCDR2, and a HCDR3 as set forth in a VH comprising the amino acid sequence of SEQ ID NO: 445, and a LCDR1, a LCDR2, and a LCDR3 as set forth in a VL comprising the amino acid sequence of SEQ ID NO: 446.

In some embodiments, the antibody or antigen binding fragment provided herein comprises a HCDR1, a HCDR2, and/or a HCDR3 as set forth in a VH comprising the amino acid sequence of SEQ ID NO: 447, and/or a LCDR1, a LCDR2, and/or a LCDR3 as set forth in a VL comprising the amino acid sequence of SEQ ID NO: 448. In some embodiments, the antibody or antigen binding fragment provided herein comprises a HCDR1, a HCDR2, and a HCDR3 as set forth in a VH comprising the amino acid sequence of SEQ ID NO: 447, and a LCDR1, a LCDR2, and a LCDR3 as set forth in a VL comprising the amino acid sequence of SEQ ID NO: 448.

In some embodiments, the antibody or antigen binding fragment provided herein comprises a HCDR1, a HCDR2, and/or a HCDR3 as set forth in a VH comprising the amino acid sequence of SEQ ID NO: 449, and/or a LCDR1, a LCDR2, and/or a LCDR3 as set forth in a VL comprising the amino acid sequence of SEQ ID NO: 450. In some embodiments, the antibody or antigen binding fragment provided herein comprises a HCDR1, a HCDR2, and a HCDR3 as set forth in a VH comprising the amino acid sequence of SEQ ID NO: 449, and a LCDR1, a LCDR2, and a LCDR3 as set forth in a VL comprising the amino acid sequence of SEQ ID NO: 450.

In some embodiments, the antibody or antigen binding fragment provided herein comprises a HCDR1, a HCDR2, and/or a HCDR3 as set forth in a VH comprising the amino acid sequence of SEQ ID NO: 451, and/or a LCDR1, a LCDR2, and/or a LCDR3 as set forth in a VL comprising the amino acid sequence of SEQ ID NO: 452. In some embodiments, the antibody or antigen binding fragment provided herein comprises a HCDR1, a HCDR2, and a HCDR3 as set forth in a VH comprising the amino acid sequence of SEQ ID NO: 451, and a LCDR1, a LCDR2, and a LCDR3 as set forth in a VL comprising the amino acid sequence of SEQ ID NO: 452.

In some embodiments, the antibody or antigen binding fragment provided herein comprises a HCDR1, a HCDR2, and/or a HCDR3 as set forth in a VH comprising the amino acid sequence of SEQ ID NO: 453, and/or a LCDR1, a LCDR2, and/or a LCDR3 as set forth in a VL comprising the amino acid sequence of SEQ ID NO: 454. In some embodiments, the antibody or antigen binding fragment provided herein comprises a HCDR1, a HCDR2, and a HCDR3 as set forth in a VH comprising the amino acid sequence of SEQ ID NO: 453, and a LCDR1, a LCDR2, and a LCDR3 as set forth in a VL comprising the amino acid sequence of SEQ ID NO: 454.

In some embodiments, the antibody or antigen binding fragment provided herein comprises a HCDR1, a HCDR2, and/or a HCDR3 as set forth in a VH comprising the amino acid sequence of SEQ ID NO: 455, and/or a LCDR1, a LCDR2, and/or a LCDR3 as set forth in a VL comprising the amino acid sequence of SEQ ID NO: 456. In some embodiments, the antibody or antigen binding fragment provided herein comprises a HCDR1, a HCDR2, and a HCDR3 as set forth in a VH comprising the amino acid sequence of SEQ ID NO: 455, and a LCDR1, a LCDR2, and a LCDR3 as set forth in a VL comprising the amino acid sequence of SEQ ID NO: 456.

In some embodiments, the antibody or antigen binding fragment provided herein comprises a HCDR1, a HCDR2, and/or a HCDR3 as set forth in a VH comprising the amino acid sequence of SEQ ID NO: 457, and/or a LCDR1, a LCDR2, and/or a LCDR3 as set forth in a VL comprising the amino acid sequence of SEQ ID NO: 458. In some embodiments, the antibody or antigen binding fragment provided herein comprises a HCDR1, a HCDR2, and a HCDR3 as set forth in a VH comprising the amino acid sequence of SEQ ID NO: 457, and a LCDR1, a LCDR2, and a LCDR3 as set forth in a VL comprising the amino acid sequence of SEQ ID NO: 458.

In some embodiments, the antibody or antigen binding fragment provided herein sequence of SEQ ID NO: 459, and/or a LCDR1, a LCDR2, and/or a LCDR3 as set forth in a VL comprising the amino acid sequence of SEQ ID NO: 460. In some embodiments, the antibody or antigen binding fragment provided herein comprises a HCDR1, a HCDR2, and a HCDR3 as set forth in a VH comprising the amino acid sequence of SEQ ID NO: 459, and a LCDR1, a LCDR2, and a LCDR3 as set forth in a VL comprising the amino acid sequence of SEQ ID NO: 460.

In some embodiments, the antibody or antigen binding fragment provided herein comprises a HCDR1, a HCDR2, and/or a HCDR3 as set forth in a VH comprising the amino acid sequence of SEQ ID NO: 461, and/or a LCDR1, a LCDR2, and/or a LCDR3 as set forth in a VL comprising the amino acid sequence of SEQ ID NO: 462. In some embodiments, the antibody or antigen binding fragment provided herein comprises a HCDR1, a HCDR2, and a HCDR3 as set forth in a VH comprising the amino acid sequence of SEQ ID NO: 461, and a LCDR1, a LCDR2, and a LCDR3 as set forth in a VL comprising the amino acid sequence of SEQ ID NO: 462.

In some embodiments, the antibody or antigen binding fragment provided herein comprises a HCDR1, a HCDR2, and/or a HCDR3 as set forth in a VH comprising the amino acid sequence of SEQ ID NO: 463, and/or a LCDR1, a LCDR2, and/or a LCDR3 as set forth in a VL comprising the amino acid sequence of SEQ ID NO: 464. In some embodiments, the antibody or antigen binding fragment provided herein comprises a HCDR1, a HCDR2, and a HCDR3 as set forth in a VH comprising the amino acid sequence of SEQ ID NO: 463, and a LCDR1, a LCDR2, and a LCDR3 as set forth in a VL comprising the amino acid sequence of SEQ ID NO: 464.

In some embodiments, the antibody or antigen binding fragment provided herein comprises a HCDR1, a HCDR2, and/or a HCDR3 as set forth in a VH comprising the amino acid sequence of SEQ ID NO: 465, and/or a LCDR1, a LCDR2, and/or a LCDR3 as set forth in a VL comprising the amino acid sequence of SEQ ID NO: 466. In some embodiments, the antibody or antigen binding fragment provided herein comprises a HCDR1, a HCDR2, and a HCDR3 as set forth in a VH comprising the amino acid sequence of SEQ ID NO: 465, and a LCDR1, a LCDR2, and a LCDR3 as set forth in a VL comprising the amino acid sequence of SEQ ID NO: 466.

In some embodiments, the antibody or antigen binding fragment provided herein comprises a HCDR1, a HCDR2, and/or a HCDR3 as set forth in a VH comprising the amino acid sequence of SEQ ID NO: 467, and/or a LCDR1, a LCDR2, and/or a LCDR3 as set forth in a VL comprising the amino acid sequence of SEQ ID NO: 468. In some embodiments, the antibody or antigen binding fragment provided herein comprises a HCDR1, a HCDR2, and a HCDR3 as set forth in a VH comprising the amino acid sequence of SEQ ID NO: 467, and a LCDR1, a LCDR2, and a LCDR3 as set forth in a VL comprising the amino acid sequence of SEQ ID NO: 468.

In some embodiments, the antibody or antigen binding fragment provided herein comprises a HCDR1, a HCDR2, and/or a HCDR3 as set forth in a VH comprising the amino acid sequence of SEQ ID NO: 469, and/or a LCDR1, a LCDR2, and/or a LCDR3 as set forth in a VL comprising the amino acid sequence of SEQ ID NO: 470. In some embodiments, the antibody or antigen binding fragment provided herein comprises a HCDR1, a HCDR2, and a HCDR3 as set forth in a VH comprising the amino acid sequence of SEQ ID NO: 469, and a LCDR1, a LCDR2, and a LCDR3 as set forth in a VL comprising the amino acid sequence of SEQ ID NO: 470.

In some embodiments, the antibody or antigen binding fragment provided herein comprises a HCDR1, a HCDR2, and/or a HCDR3 as set forth in a VH comprising the amino acid sequence of SEQ ID NO: 471, and/or a LCDR1, a LCDR2, and/or a LCDR3 as set forth in a VL comprising the amino acid sequence of SEQ ID NO: 472. In some embodiments, the antibody or antigen binding fragment provided herein comprises a HCDR1, a HCDR2, and a HCDR3 as set forth in a VH comprising the amino acid sequence of SEQ ID NO: 471, and a LCDR1, a LCDR2, and a LCDR3 as set forth in a VL comprising the amino acid sequence of SEQ ID NO: 472.

In some embodiments, the antibody or antigen binding fragment provided herein comprises a HCDR1, a HCDR2, and/or a HCDR3 as set forth in a VH comprising the amino acid sequence of SEQ ID NO: 473, and/or a LCDR1, a LCDR2, and/or a LCDR3 as set forth in a VL comprising the amino acid sequence of SEQ ID NO: 474. In some embodiments, the antibody or antigen binding fragment provided herein comprises a HCDR1, a HCDR2, and a HCDR3 as set forth in a VH comprising the amino acid sequence of SEQ ID NO: 473, and a LCDR1, a LCDR2, and a LCDR3 as set forth in a VL comprising the amino acid sequence of SEQ ID NO: 474.

In some embodiments, the antibody or antigen binding fragment provided herein comprises a HCDR1, a HCDR2, and/or a HCDR3 as set forth in a VH comprising the amino acid sequence of SEQ ID NO: 475, and/or a LCDR1, a LCDR2, and/or a LCDR3 as set forth in a VL comprising the amino acid sequence of SEQ ID NO: 476. In some embodiments, the antibody or antigen binding fragment provided herein comprises a HCDR1, a HCDR2, and a HCDR3 as set forth in a VH comprising the amino acid sequence of SEQ ID NO: 475, and a LCDR1, a LCDR2, and a LCDR3 as set forth in a VL comprising the amino acid sequence of SEQ ID NO: 476.

In some embodiments, the antibody or antigen binding fragment provided herein sequence of SEQ ID NO: 477, and/or a LCDR1, a LCDR2, and/or a LCDR3 as set forth in a VL comprising the amino acid sequence of SEQ ID NO: 478. In some embodiments, the antibody or antigen binding fragment provided herein comprises a HCDR1, a HCDR2, and a HCDR3 as set forth in a VH comprising the amino acid sequence of SEQ ID NO: 477, and a LCDR1, a LCDR2, and a LCDR3 as set forth in a VL comprising the amino acid sequence of SEQ ID NO: 478.

In some embodiments, the antibody or antigen binding fragment provided herein comprises a HCDR1, a HCDR2, and/or a HCDR3 as set forth in a VH comprising the amino acid sequence of SEQ ID NO: 479, and/or a LCDR1, a LCDR2, and/or a LCDR3 as set forth in a VL comprising the amino acid sequence of SEQ ID NO: 480. In some embodiments, the antibody or antigen binding fragment provided herein comprises a HCDR1, a HCDR2, and a HCDR3 as set forth in a VH comprising the amino acid sequence of SEQ ID NO: 479, and a LCDR1, a LCDR2, and a LCDR3 as set forth in a VL comprising the amino acid sequence of SEQ ID NO: 480.

In some embodiments, the antibody or antigen binding fragment provided herein comprises a HCDR1, a HCDR2, and/or a HCDR3 as set forth in a VH comprising the amino acid sequence of SEQ ID NO: 481, and/or a LCDR1, a LCDR2, and/or a LCDR3 as set forth in a VL comprising the amino acid sequence of SEQ ID NO: 482. In some embodiments, the antibody or antigen binding fragment provided herein comprises a HCDR1, a HCDR2, and a HCDR3 as set forth in a VH comprising the amino acid sequence of SEQ ID NO: 481, and a LCDR1, a LCDR2, and a LCDR3 as set forth in a VL comprising the amino acid sequence of SEQ ID NO: 482.

In some embodiments, the antibody or antigen binding fragment provided herein comprises a HCDR1, a HCDR2, and/or a HCDR3 as set forth in a VH comprising the amino acid sequence of SEQ ID NO: 483, and/or a LCDR1, a LCDR2, and/or a LCDR3 as set forth in a VL comprising the amino acid sequence of SEQ ID NO: 484. In some embodiments, the antibody or antigen binding fragment provided herein comprises a HCDR1, a HCDR2, and a HCDR3 as set forth in a VH comprising the amino acid sequence of SEQ ID NO: 483, and a LCDR1, a LCDR2, and a LCDR3 as set forth in a VL comprising the amino acid sequence of SEQ ID NO: 484.

In some embodiments, the antibody or antigen binding fragment provided herein comprises a HCDR1, a HCDR2, and/or a HCDR3 as set forth in a VH comprising the amino acid sequence of SEQ ID NO: 485, and/or a LCDR1, a LCDR2, and/or a LCDR3 as set forth in a VL comprising the amino acid sequence of SEQ ID NO: 486. In some embodiments, the antibody or antigen binding fragment provided herein comprises a HCDR1, a HCDR2, and a HCDR3 as set forth in a VH comprising the amino acid sequence of SEQ ID NO: 485, and a LCDR1, a LCDR2, and a LCDR3 as set forth in a VL comprising the amino acid sequence of SEQ ID NO: 486.

In some embodiments, the antibody or antigen binding fragment provided herein comprises a HCDR1, a HCDR2, and/or a HCDR3 as set forth in a VH comprising the amino acid sequence of SEQ ID NO: 487, and/or a LCDR1, a LCDR2, and/or a LCDR3 as set forth in a VL comprising the amino acid sequence of SEQ ID NO: 488. In some embodiments, the antibody or antigen binding fragment provided herein comprises a HCDR1, a HCDR2, and a HCDR3 as set forth in a VH comprising the amino acid sequence of SEQ ID NO: 487, and a LCDR1, a LCDR2, and a LCDR3 as set forth in a VL comprising the amino acid sequence of SEQ ID NO: 488.

In some embodiments, the antibody or antigen binding fragment provided herein comprises a HCDR1, a HCDR2, and/or a HCDR3 as set forth in a VH comprising the amino acid sequence of SEQ ID NO: 489, and/or a LCDR1, a LCDR2, and/or a LCDR3 as set forth in a VL comprising the amino acid sequence of SEQ ID NO: 490. In some embodiments, the antibody or antigen binding fragment provided herein comprises a HCDR1, a HCDR2, and a HCDR3 as set forth in a VH comprising the amino acid sequence of SEQ ID NO: 489, and a LCDR1, a LCDR2, and a LCDR3 as set forth in a VL comprising the amino acid sequence of SEQ ID NO: 490.

In some embodiments, the antibody or antigen binding fragment provided herein comprises a HCDR1, a HCDR2, and/or a HCDR3 as set forth in a VH comprising the amino acid sequence of SEQ ID NO: 491, and/or a LCDR1, a LCDR2, and/or a LCDR3 as set forth in a VL comprising the amino acid sequence of SEQ ID NO: 492. In some embodiments, the antibody or antigen binding fragment provided herein comprises a HCDR1, a HCDR2, and a HCDR3 as set forth in a VH comprising the amino acid sequence of SEQ ID NO: 491, and a LCDR1, a LCDR2, and a LCDR3 as set forth in a VL comprising the amino acid sequence of SEQ ID NO: 492.

In some embodiments, the antibody or antigen binding fragment provided herein comprises a HCDR1, a HCDR2, and/or a HCDR3 as set forth in a VH comprising the amino acid sequence of SEQ ID NO: 493, and/or a LCDR1, a LCDR2, and/or a LCDR3 as set forth in a VL comprising the amino acid sequence of SEQ ID NO: 494. In some embodiments, the antibody or antigen binding fragment provided herein comprises a HCDR1, a HCDR2, and a HCDR3 as set forth in a VH comprising the amino acid sequence of SEQ ID NO: 493, and a LCDR1, a LCDR2, and a LCDR3 as set forth in a VL comprising the amino acid sequence of SEQ ID NO: 494.

In some embodiments, the antibody or antigen binding fragment provided herein sequence of SEQ ID NO: 1167, and/or a LCDR1, a LCDR2, and/or a LCDR3 as set forth in a VL comprising the amino acid sequence of SEQ ID NO: 1168. In some embodiments, the antibody or antigen binding fragment provided herein comprises a HCDR1, a HCDR2, and a HCDR3 as set forth in a VH comprising the amino acid sequence of SEQ ID NO: 1167, and a LCDR1, a LCDR2, and a LCDR3 as set forth in a VL comprising the amino acid sequence of SEQ ID NO: 1168.

In some embodiments, the antibody or antigen binding fragment provided herein comprises a HCDR1, a HCDR2, and/or a HCDR3 as set forth in a VH comprising the amino acid sequence of SEQ ID NO: 1169, and/or a LCDR1, a LCDR2, and/or a LCDR3 as set forth in a VL comprising the amino acid sequence of SEQ ID NO: 1170. In some embodiments, the antibody or antigen binding fragment provided herein comprises a HCDR1, a HCDR2, and a HCDR3 as set forth in a VH comprising the amino acid sequence of SEQ ID NO: 1169, and a LCDR1, a LCDR2, and a LCDR3 as set forth in a VL comprising the amino acid sequence of SEQ ID NO: 1170.

In some embodiments, the antibody or antigen binding fragment provided herein comprises a HCDR1, a HCDR2, and/or a HCDR3 as set forth in a VH comprising the amino acid sequence of SEQ ID NO: 1171, and/or a LCDR1, a LCDR2, and/or a LCDR3 as set forth in a VL comprising the amino acid sequence of SEQ ID NO: 1172. In some embodiments, the antibody or antigen binding fragment provided herein comprises a HCDR1, a HCDR2, and a HCDR3 as set forth in a VH comprising the amino acid sequence of SEQ ID NO: 1171, and a LCDR1, a LCDR2, and a LCDR3 as set forth in a VL comprising the amino acid sequence of SEQ ID NO: 1172.

In some embodiments, the antibody or antigen binding fragment provided herein comprises a HCDR1, a HCDR2, and/or a HCDR3 as set forth in a VH comprising the amino acid sequence of SEQ ID NO: 1173, and/or a LCDR1, a LCDR2, and/or a LCDR3 as set forth in a VL comprising the amino acid sequence of SEQ ID NO: 1174. In some embodiments, the antibody or antigen binding fragment provided herein comprises a HCDR1, a HCDR2, and a HCDR3 as set forth in a VH comprising the amino acid sequence of SEQ ID NO: 1173, and a LCDR1, a LCDR2, and a LCDR3 as set forth in a VL comprising the amino acid sequence of SEQ ID NO: 1174.

In some embodiments, the antibody or antigen binding fragment provided herein comprises a HCDR1, a HCDR2, and/or a HCDR3 as set forth in a VH comprising the amino acid sequence of SEQ ID NO: 1175, and/or a LCDR1, a LCDR2, and/or a LCDR3 as set forth in a VL comprising the amino acid sequence of SEQ ID NO: 1176. In some embodiments, the antibody or antigen binding fragment provided herein comprises a HCDR1, a HCDR2, and a HCDR3 as set forth in a VH comprising the amino acid sequence of SEQ ID NO: 1175, and a LCDR1, a LCDR2, and a LCDR3 as set forth in a VL comprising the amino acid sequence of SEQ ID NO: 1176.

In some embodiments, the antibody or antigen binding fragment provided herein comprises a HCDR1, a HCDR2, and/or a HCDR3 as set forth in a VH comprising the amino acid sequence of SEQ ID NO: 1177, and/or a LCDR1, a LCDR2, and/or a LCDR3 as set forth in a VL comprising the amino acid sequence of SEQ ID NO: 1178. In some embodiments, the antibody or antigen binding fragment provided herein comprises a HCDR1, a HCDR2, and a HCDR3 as set forth in a VH comprising the amino acid sequence of SEQ ID NO: 1177, and a LCDR1, a LCDR2, and a LCDR3 as set forth in a VL comprising the amino acid sequence of SEQ ID NO: 1178.

CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering.

In other embodiments, provided herein is an antibody that binds to LILRB2 comprising (a) a VH region comprising (i) a HCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NOs: 849, 855, 861, 867, 873, 879, 885, 891, 897, 903, 909, 915, 921, 927, 933, 939, 945, 951, 957, 963, 969, 975, 981, 987, 993, 999, 1005, 1011, 1017, 1023, 1029, 1035, 1041, 1047, 1053, 1059, 1065, 1071, 1077, 1083, 1089, 1095, 1101, 1107, 1113, 1119, and 1125; (ii) a HCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NOs: 850, 856, 862, 868, 874, 880, 886, 892, 898, 904, 910, 916, 922, 928, 934, 940, 946, 952, 958, 964, 970, 976, 982, 988, 994, 1000, 1006, 1012, 1018, 1024, 1030, 1036, 1042, 1048, 1054, 1060, 1066, 1072, 1078, 1084, 1090, 1096, 1102, 1108, 1114, 1120, and 1126, (iii) a HCDR3 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 851, 857, 863, 869, 875, 881, 887, 893, 899, 905, 911, 917, 923, 929, 935, 941, 947, 953, 959, 965, 971, 977, 983, 989, 995, 1001, 1007, 1013, 1019, 1025, 1031, 1037, 1043, 1049, 1055, 1061, 1067, 1073, 1079, 1085, 1091, 1097, 1103, 1109, 1115, 1121, and 1127; and/or (b) a VL region comprising (i) a LCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NOs: 852, 858, 864, 870, 876, 882, 888, 894, 900, 906, 912, 918, 924, 930, 936, 942, 948, 954, 960, 966, 972, 978, 984, 990, 996, 1002, 1008, 1014, 1020, 1026, 1032, 1038, 1044, 1050, 1056, 1062, 1068, 1074, 1080, 1086, 1092, 1098, 1104, 1110, 1116, 1122, and 1128; (ii) a LCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any of SEQ ID NOs: 853, 859, 865, 871, 877, 883, 889, 895, 901, 907, 913, 919, 925, 931, 937, 943, 949, 955, 961, 967, 973, 979, 985, 991, 997, 1003, 1009, 1015, 1021, 1027, 1033, 1039 1045, 1051, 1057, 1063, 1069, 1075, 1081, 1087, 1093, 1099, 1105, 1111, 1117, 1123, 1129, and 1282; and (iii) a LCDR3 comprising an amino acid sequence having at least 75%, 80%, 85%, 86%, 87%, 88%, 89%, 90%, 91%, 92%, 93%, 94%, 95%, 96%, 97%, 98%, 99%, 100% sequence identity to any of SEQ ID NOs: 854, 860, 866, 872, 878, 884, 890, 896, 902, 908, 914, 920, 926, 932, 938, 944, 950, 956, 962, 968, 974, 980, 986, 992, 998, 1004, 1010, 1016, 1022, 1028, 1034, 1040, 1046, 1052, 1058, 1064, 1070, 1076, 1082, 1088, 1094, 1100, 1106, 1112, 1118, 1124, and 1130. In some embodiments, the anti-LILRB2 antibody is humanized. In some embodiments, the anti-LILRB2 antibody comprises an acceptor human framework, e.g., a human immunoglobulin framework or a human consensus framework.

In some specific embodiments, in the antibody or antigen binding fragment provided herein, the HCDR1 comprises the amino acid sequence of SEQ ID NO: 849, the HCDR2 comprises the amino acid sequence of SEQ ID NO: 850, the HCDR3 comprises the amino acid sequence of SEQ ID NO: 851, the LCDR1 comprises the amino acid sequence of SEQ ID NO: 852, the LCDR2 comprises the amino acid sequence of SEQ ID NO: 853, and the LCDR3 comprises the amino acid sequence of SEQ ID NO: 854.

In some specific embodiments, in the antibody or antigen binding fragment provided herein, the HCDR1 comprises the amino acid sequence of SEQ ID NO: 855, the HCDR2 comprises the amino acid sequence of SEQ ID NO: 856, the HCDR3 comprises the amino acid sequence of SEQ ID NO: 857, the LCDR1 comprises the amino acid sequence of SEQ ID NO: 858, the LCDR2 comprises the amino acid sequence of SEQ ID NO: 859, and the LCDR3 comprises the amino acid sequence of SEQ ID NO: 860.

In some specific embodiments, in the antibody or antigen binding fragment provided herein, the HCDR1 comprises the amino acid sequence of SEQ ID NO: 861, the HCDR2 comprises the amino acid sequence of SEQ ID NO: 862, the HCDR3 comprises the amino acid sequence of SEQ ID NO: 863, the LCDR1 comprises the amino acid sequence of SEQ ID NO: 864, the LCDR2 comprises the amino acid sequence of SEQ ID NO: 865, and the LCDR3 comprises the amino acid sequence of SEQ ID NO: 866.

In some specific embodiments, in the antibody or antigen binding fragment provided herein, the HCDR1 comprises the amino acid sequence of SEQ ID NO: 867, the HCDR2 comprises the amino acid sequence of SEQ ID NO: 868, the HCDR3 comprises the amino acid sequence of SEQ ID NO: 869, the LCDR1 comprises the amino acid sequence of SEQ ID NO: 870, the LCDR2 comprises the amino acid sequence of SEQ ID NO: 871, and the LCDR3 comprises the amino acid sequence of SEQ ID NO: 872.

In some specific embodiments, in the antibody or antigen binding fragment provided herein, the HCDR1 comprises the amino acid sequence of SEQ ID NO: 873, the HCDR2 comprises the amino acid sequence of SEQ ID NO: 874, the HCDR3 comprises the amino acid sequence of SEQ ID NO: 875, the LCDR1 comprises the amino acid sequence of SEQ ID NO: 876, the LCDR2 comprises the amino acid sequence of SEQ ID NO: 877, and the LCDR3 comprises the amino acid sequence of SEQ ID NO: 878.

In some specific embodiments, in the antibody or antigen binding fragment provided herein, the HCDR1 comprises the amino acid sequence of SEQ ID NO: 879, the HCDR2 comprises the amino acid sequence of SEQ ID NO: 880, the HCDR3 comprises the amino acid sequence of SEQ ID NO: 881, the LCDR1 comprises the amino acid sequence of SEQ ID NO: 882, the LCDR2 comprises the amino acid sequence of SEQ ID NO: 883, and the LCDR3 comprises the amino acid sequence of SEQ ID NO: 884.

In some specific embodiments, in the antibody or antigen binding fragment provided herein, the HCDR1 comprises the amino acid sequence of SEQ ID NO: 885, the HCDR2 comprises the amino acid sequence of SEQ ID NO: 886, the HCDR3 comprises the amino acid sequence of SEQ ID NO: 887, the LCDR1 comprises the amino acid sequence of SEQ ID NO: 888, the LCDR2 comprises the amino acid sequence of SEQ ID NO: 889, and the LCDR3 comprises the amino acid sequence of SEQ ID NO: 890.

In some specific embodiments, in the antibody or antigen binding fragment provided herein, the HCDR1 comprises the amino acid sequence of SEQ ID NO: 891, the HCDR2 comprises the amino acid sequence of SEQ ID NO: 892, the HCDR3 comprises the amino acid sequence of SEQ ID NO: 893, the LCDR1 comprises the amino acid sequence of SEQ ID NO: 894, the LCDR2 comprises the amino acid sequence of SEQ ID NO: 895, and the LCDR3 comprises the amino acid sequence of SEQ ID NO: 896.

In some specific embodiments, in the antibody or antigen binding fragment provided herein, the HCDR1 comprises the amino acid sequence of SEQ ID NO: 897, the HCDR2 comprises the amino acid sequence of SEQ ID NO: 898, the HCDR3 comprises the amino acid sequence of SEQ ID NO: 899, the LCDR1 comprises the amino acid sequence of SEQ ID NO: 900, the LCDR2 comprises the amino acid sequence of SEQ ID NO: 901, and the LCDR3 comprises the amino acid sequence of SEQ ID NO: 902.

In some specific embodiments, in the antibody or antigen binding fragment provided herein, the HCDR1 comprises the amino acid sequence of SEQ ID NO: 903, the HCDR2 comprises the amino acid sequence of SEQ ID NO: 904, the HCDR3 comprises the amino acid sequence of SEQ ID NO: 905, the LCDR1 comprises the amino acid sequence of SEQ ID NO: 906, the LCDR2 comprises the amino acid sequence of SEQ ID NO: 907, and the LCDR3 comprises the amino acid sequence of SEQ ID NO: 908.

In some specific embodiments, in the antibody or antigen binding fragment provided herein, the HCDR1 comprises the amino acid sequence of SEQ ID NO: 909, the HCDR2 comprises the amino acid sequence of SEQ ID NO: 910, the HCDR3 comprises the amino acid sequence of SEQ ID NO: 911, the LCDR1 comprises the amino acid sequence of SEQ ID NO: 912, the LCDR2 comprises the amino acid sequence of SEQ ID NO: 913, and the LCDR3 comprises the amino acid sequence of SEQ ID NO: 914.

In some specific embodiments, in the antibody or antigen binding fragment provided herein, the HCDR1 comprises the amino acid sequence of SEQ ID NO: 915, the HCDR2 comprises the amino acid sequence of SEQ ID NO: 916, the HCDR3 comprises the amino acid sequence of SEQ ID NO: 917, the LCDR1 comprises the amino acid sequence of SEQ ID NO: 918, the LCDR2 comprises the amino acid sequence of SEQ ID NO: 919, and the LCDR3 comprises the amino acid sequence of SEQ ID NO: 920.

In some specific embodiments, in the antibody or antigen binding fragment provided herein, the HCDR1 comprises the amino acid sequence of SEQ ID NO: 921, the HCDR2 comprises the amino acid sequence of SEQ ID NO: 922, the HCDR3 comprises the amino acid sequence of SEQ ID NO: 923, the LCDR1 comprises the amino acid sequence of SEQ ID NO: 924, the LCDR2 comprises the amino acid sequence of SEQ ID NO: 925, and the LCDR3 comprises the amino acid sequence of SEQ ID NO: 926.

In some specific embodiments, in the antibody or antigen binding fragment provided herein, the HCDR1 comprises the amino acid sequence of SEQ ID NO: 927, the HCDR2 comprises the amino acid sequence of SEQ ID NO: 928, the HCDR3 comprises the amino acid sequence of SEQ ID NO: 929, the LCDR1 comprises the amino acid sequence of SEQ ID NO: 930, the LCDR2 comprises the amino acid sequence of SEQ ID NO: 931, and the LCDR3 comprises the amino acid sequence of SEQ ID NO: 932.

In some specific embodiments, in the antibody or antigen binding fragment provided herein, the HCDR1 comprises the amino acid sequence of SEQ ID NO: 933, the HCDR2 comprises the amino acid sequence of SEQ ID NO: 934, the HCDR3 comprises the amino acid sequence of SEQ ID NO: 935, the LCDR1 comprises the amino acid sequence of SEQ ID NO: 936, the LCDR2 comprises the amino acid sequence of SEQ ID NO: 937, and the LCDR3 comprises the amino acid sequence of SEQ ID NO: 938.

In some specific embodiments, in the antibody or antigen binding fragment provided herein, the HCDR1 comprises the amino acid sequence of SEQ ID NO: 939, the HCDR2 comprises the amino acid sequence of SEQ ID NO: 940, the HCDR3 comprises the amino acid sequence of SEQ ID NO: 941, the LCDR1 comprises the amino acid sequence of SEQ ID NO: 942, the LCDR2 comprises the amino acid sequence of SEQ ID NO: 943, and the LCDR3 comprises the amino acid sequence of SEQ ID NO: 944.

In some specific embodiments, in the antibody or antigen binding fragment provided herein, the HCDR1 comprises the amino acid sequence of SEQ ID NO: 945, the HCDR2 comprises the amino acid sequence of SEQ ID NO: 946, the HCDR3 comprises the amino acid sequence of SEQ ID NO: 947, the LCDR1 comprises the amino acid sequence of SEQ ID NO: 948, the LCDR2 comprises the amino acid sequence of SEQ ID NO: 949, and the LCDR3 comprises the amino acid sequence of SEQ ID NO: 950.

In some specific embodiments, in the antibody or antigen binding fragment provided herein, the HCDR1 comprises the amino acid sequence of SEQ ID NO: 951, the HCDR2 comprises the amino acid sequence of SEQ ID NO: 952, the HCDR3 comprises the amino acid sequence of SEQ ID NO: 953, the LCDR1 comprises the amino acid sequence of SEQ ID NO: 954, the LCDR2 comprises the amino acid sequence of SEQ ID NO: 955, and the LCDR3 comprises the amino acid sequence of SEQ ID NO: 956.

In some specific embodiments, in the antibody or antigen binding fragment provided herein, the HCDR1 comprises the amino acid sequence of SEQ ID NO: 957, the HCDR2 comprises the amino acid sequence of SEQ ID NO: 958, the HCDR3 comprises the amino acid sequence of SEQ ID NO: 959, the LCDR1 comprises the amino acid sequence of SEQ ID NO: 960, the LCDR2 comprises the amino acid sequence of SEQ ID NO: 961, and the LCDR3 comprises the amino acid sequence of SEQ ID NO: 962.

In some specific embodiments, in the antibody or antigen binding fragment provided herein, the HCDR1 comprises the amino acid sequence of SEQ ID NO: 963, the HCDR2 comprises the amino acid sequence of SEQ ID NO: 964, the HCDR3 comprises the amino acid sequence of SEQ ID NO: 965, the LCDR1 comprises the amino acid sequence of SEQ ID NO: 966, the LCDR2 comprises the amino acid sequence of SEQ ID NO: 967, and the LCDR3 comprises the amino acid sequence of SEQ ID NO: 968.

In some specific embodiments, in the antibody or antigen binding fragment provided herein, the HCDR1 comprises the amino acid sequence of SEQ ID NO: 969, the HCDR2 comprises the amino acid sequence of SEQ ID NO: 970, the HCDR3 comprises the amino acid sequence of SEQ ID NO: 971, the LCDR1 comprises the amino acid sequence of SEQ ID NO: 972, the LCDR2 comprises the amino acid sequence of SEQ ID NO: 973, and the LCDR3 comprises the amino acid sequence of SEQ ID NO: 974.

In some specific embodiments, in the antibody or antigen binding fragment provided herein, the HCDR1 comprises the amino acid sequence of SEQ ID NO: 975, the HCDR2 comprises the amino acid sequence of SEQ ID NO: 976, the HCDR3 comprises the amino acid sequence of SEQ ID NO: 977, the LCDR1 comprises the amino acid sequence of SEQ ID NO: 978, the LCDR2 comprises the amino acid sequence of SEQ ID NO: 979, and the LCDR3 comprises the amino acid sequence of SEQ ID NO: 980.

In some specific embodiments, in the antibody or antigen binding fragment provided herein, the HCDR1 comprises the amino acid sequence of SEQ ID NO: 981, the HCDR2 comprises the amino acid sequence of SEQ ID NO: 982, the HCDR3 comprises the amino acid sequence of SEQ ID NO: 983, the LCDR1 comprises the amino acid sequence of SEQ ID NO: 984, the LCDR2 comprises the amino acid sequence of SEQ ID NO: 985, and the LCDR3 comprises the amino acid sequence of SEQ ID NO: 986.

In some specific embodiments, in the antibody or antigen binding fragment provided herein, the HCDR1 comprises the amino acid sequence of SEQ ID NO: 987, the HCDR2 comprises the amino acid sequence of SEQ ID NO: 988, the HCDR3 comprises the amino acid sequence of SEQ ID NO: 989, the LCDR1 comprises the amino acid sequence of SEQ ID NO: 990, the LCDR2 comprises the amino acid sequence of SEQ ID NO: 991, and the LCDR3 comprises the amino acid sequence of SEQ ID NO: 992.

In some specific embodiments, in the antibody or antigen binding fragment provided herein, the HCDR1 comprises the amino acid sequence of SEQ ID NO: 993, the HCDR2 comprises the amino acid sequence of SEQ ID NO: 994, the HCDR3 comprises the amino acid sequence of SEQ ID NO: 995, the LCDR1 comprises the amino acid sequence of SEQ ID NO: 996, the LCDR2 comprises the amino acid sequence of SEQ ID NO: 997, and the LCDR3 comprises the amino acid sequence of SEQ ID NO: 998.

In some specific embodiments, in the antibody or antigen binding fragment provided herein, the HCDR1 comprises the amino acid sequence of SEQ ID NO: 999, the HCDR2 comprises the amino acid sequence of SEQ ID NO: 1000, the HCDR3 comprises the amino acid sequence of SEQ ID NO: 1001, the LCDR1 comprises the amino acid sequence of SEQ ID NO: 1002, the LCDR2 comprises the amino acid sequence of SEQ ID NO: 1003, and the LCDR3 comprises the amino acid sequence of SEQ ID NO: 1004.

In some specific embodiments, in the antibody or antigen binding fragment provided herein, the HCDR1 comprises the amino acid sequence of SEQ ID NO: 1005, the HCDR2 comprises the amino acid sequence of SEQ ID NO: 1006, the HCDR3 comprises the amino acid sequence of SEQ ID NO: 1007, the LCDR1 comprises the amino acid sequence of SEQ ID NO: 1008, the LCDR2 comprises the amino acid sequence of SEQ ID NO: 1009, and the LCDR3 comprises the amino acid sequence of SEQ ID NO: 1010.

In some specific embodiments, in the antibody or antigen binding fragment provided herein, the HCDR1 comprises the amino acid sequence of SEQ ID NO: 1011, the HCDR2 comprises the amino acid sequence of SEQ ID NO: 1012, the HCDR3 comprises the amino acid sequence of SEQ ID NO: 1013, the LCDR1 comprises the amino acid sequence of SEQ ID NO: 1014, the LCDR2 comprises the amino acid sequence of SEQ ID NO: 1015, and the LCDR3 comprises the amino acid sequence of SEQ ID NO: 1016.

In some specific embodiments, in the antibody or antigen binding fragment provided herein, the HCDR1 comprises the amino acid sequence of SEQ ID NO: 1017, the HCDR2 comprises the amino acid sequence of SEQ ID NO: 1018, the HCDR3 comprises the amino acid sequence of SEQ ID NO: 1019, the LCDR1 comprises the amino acid sequence of SEQ ID NO: 1020, the LCDR2 comprises the amino acid sequence of SEQ ID NO: 1021, and the LCDR3 comprises the amino acid sequence of SEQ ID NO: 1022.

In some specific embodiments, in the antibody or antigen binding fragment provided herein, the HCDR1 comprises the amino acid sequence of SEQ ID NO: 1023, the HCDR2 comprises the amino acid sequence of SEQ ID NO: 1024, the HCDR3 comprises the amino acid sequence of SEQ ID NO: 1025, the LCDR1 comprises the amino acid sequence of SEQ ID NO: 1026, the LCDR2 comprises the amino acid sequence of SEQ ID NO: 1027, and the LCDR3 comprises the amino acid sequence of SEQ ID NO: 1028.

In some specific embodiments, in the antibody or antigen binding fragment provided herein, the HCDR1 comprises the amino acid sequence of SEQ ID NO: 1029, the HCDR2 comprises the amino acid sequence of SEQ ID NO: 1030, the HCDR3 comprises the amino acid sequence of SEQ ID NO: 1031, the LCDR1 comprises the amino acid sequence of SEQ ID NO: 1032, the LCDR2 comprises the amino acid sequence of SEQ ID NO: 1033, and the LCDR3 comprises the amino acid sequence of SEQ ID NO: 1034.

In some specific embodiments, in the antibody or antigen binding fragment provided herein, the HCDR1 comprises the amino acid sequence of SEQ ID NO: 1035, the HCDR2 comprises the amino acid sequence of SEQ ID NO: 1036, the HCDR3 comprises the amino acid sequence of SEQ ID NO: 1037, the LCDR1 comprises the amino acid sequence of SEQ ID NO: 1038, the LCDR2 comprises the amino acid sequence of SEQ ID NO: 1039, and the LCDR3 comprises the amino acid sequence of SEQ ID NO: 1040.

In some specific embodiments, in the antibody or antigen binding fragment provided herein, the HCDR1 comprises the amino acid sequence of SEQ ID NO: 1041, the HCDR2 comprises the amino acid sequence of SEQ ID NO: 1042, the HCDR3 comprises the amino acid sequence of SEQ ID NO: 1043, the LCDR1 comprises the amino acid sequence of SEQ ID NO: 1044, the LCDR2 comprises the amino acid sequence of SEQ ID NO: 1045, and the LCDR3 comprises the amino acid sequence of SEQ ID NO: 1046.

In some specific embodiments, in the antibody or antigen binding fragment provided herein, the HCDR1 comprises the amino acid sequence of SEQ ID NO: 1047, the HCDR2 comprises the amino acid sequence of SEQ ID NO: 1048, the HCDR3 comprises the amino acid sequence of SEQ ID NO: 1049, the LCDR1 comprises the amino acid sequence of SEQ ID NO: 1050, the LCDR2 comprises the amino acid sequence of SEQ ID NO: 1051, and the LCDR3 comprises the amino acid sequence of SEQ ID NO: 1052.

In some specific embodiments, in the antibody or antigen binding fragment provided herein, the HCDR1 comprises the amino acid sequence of SEQ ID NO: 1053, the HCDR2 comprises the amino acid sequence of SEQ ID NO: 1054, the HCDR3 comprises the amino acid sequence of SEQ ID NO: 1055, the LCDR1 comprises the amino acid sequence of SEQ ID NO: 1056, the LCDR2 comprises the amino acid sequence of SEQ ID NO: 1057, and the LCDR3 comprises the amino acid sequence of SEQ ID NO: 1058.

In some specific embodiments, in the antibody or antigen binding fragment provided herein, the HCDR1 comprises the amino acid sequence of SEQ ID NO: 1059, the HCDR2 comprises the amino acid sequence of SEQ ID NO: 1060, the HCDR3 comprises the amino acid sequence of SEQ ID NO: 1061, the LCDR1 comprises the amino acid sequence of SEQ ID NO: 1062, the LCDR2 comprises the amino acid sequence of SEQ ID NO: 1063, and the LCDR3 comprises the amino acid sequence of SEQ ID NO: 1064.

In some specific embodiments, in the antibody or antigen binding fragment provided herein, the HCDR1 comprises the amino acid sequence of SEQ ID NO: 1065, the HCDR2 comprises the amino acid sequence of SEQ ID NO: 1066, the HCDR3 comprises the amino acid sequence of SEQ ID NO: 1067, the LCDR1 comprises the amino acid sequence of SEQ ID NO: 1068, the LCDR2 comprises the amino acid sequence of SEQ ID NO: 1069, and the LCDR3 comprises the amino acid sequence of SEQ ID NO: 1070.

In some specific embodiments, in the antibody or antigen binding fragment provided herein, the HCDR1 comprises the amino acid sequence of SEQ ID NO: 1071, the HCDR2 comprises the amino acid sequence of SEQ ID NO: 1072, the HCDR3 comprises the amino acid sequence of SEQ ID NO: 1073, the LCDR1 comprises the amino acid sequence of SEQ ID NO: 1074, the LCDR2 comprises the amino acid sequence of SEQ ID NO: 1075, and the LCDR3 comprises the amino acid sequence of SEQ ID NO: 1076.

In some specific embodiments, in the antibody or antigen binding fragment provided herein, the HCDR1 comprises the amino acid sequence of SEQ ID NO: 1077, the HCDR2 comprises the amino acid sequence of SEQ ID NO: 1078, the HCDR3 comprises the amino acid sequence of SEQ ID NO: 1079, the LCDR1 comprises the amino acid sequence of SEQ ID NO: 1080, the LCDR2 comprises the amino acid sequence of SEQ ID NO: 1081, and the LCDR3 comprises the amino acid sequence of SEQ ID NO: 1082.

In some specific embodiments, in the antibody or antigen binding fragment provided herein, the HCDR1 comprises the amino acid sequence of SEQ ID NO: 1083, the HCDR2 comprises the amino acid sequence of SEQ ID NO: 1084, the HCDR3 comprises the amino acid sequence of SEQ ID NO: 1085, the LCDR1 comprises the amino acid sequence of SEQ ID NO: 1086, the LCDR2 comprises the amino acid sequence of SEQ ID NO: 1087, and the LCDR3 comprises the amino acid sequence of SEQ ID NO: 1088.

In some specific embodiments, in the antibody or antigen binding fragment provided herein, the HCDR1 comprises the amino acid sequence of SEQ ID NO: 1089, the HCDR2 comprises the amino acid sequence of SEQ ID NO: 1090, the HCDR3 comprises the amino acid sequence of SEQ ID NO: 1091, the LCDR1 comprises the amino acid sequence of SEQ ID NO: 1092, the LCDR2 comprises the amino acid sequence of SEQ ID NO: 1093, and the LCDR3 comprises the amino acid sequence of SEQ ID NO: 1094.

In some specific embodiments, in the antibody or antigen binding fragment provided herein, the HCDR1 comprises the amino acid sequence of SEQ ID NO: 1095, the HCDR2 comprises the amino acid sequence of SEQ ID NO: 1096, the HCDR3 comprises the amino acid sequence of SEQ ID NO: 1097, the LCDR1 comprises the amino acid sequence of SEQ ID NO: 1098, the LCDR2 comprises the amino acid sequence of SEQ ID NO: 1099, and the LCDR3 comprises the amino acid sequence of SEQ ID NO: 1100.

In some specific embodiments, in the antibody or antigen binding fragment provided herein, the HCDR1 comprises the amino acid sequence of SEQ ID NO: 1101, the HCDR2 comprises the amino acid sequence of SEQ ID NO: 1102, the HCDR3 comprises the amino acid sequence of SEQ ID NO: 1103, the LCDR1 comprises the amino acid sequence of SEQ ID NO: 1104, the LCDR2 comprises the amino acid sequence of SEQ ID NO: 1105, and the LCDR3 comprises the amino acid sequence of SEQ ID NO: 1106.

In some specific embodiments, in the antibody or antigen binding fragment provided herein, the HCDR1 comprises the amino acid sequence of SEQ ID NO: 1107, the HCDR2 comprises the amino acid sequence of SEQ ID NO: 1108, the HCDR3 comprises the amino acid sequence of SEQ ID NO: 1109, the LCDR1 comprises the amino acid sequence of SEQ ID NO: 1110, the LCDR2 comprises the amino acid sequence of SEQ ID NO: 1111, and the LCDR3 comprises the amino acid sequence of SEQ ID NO: 1112.

In some specific embodiments, in the antibody or antigen binding fragment provided herein, the HCDR1 comprises the amino acid sequence of SEQ ID NO: 1113, the HCDR2 comprises the amino acid sequence of SEQ ID NO: 1114, the HCDR3 comprises the amino acid sequence of SEQ ID NO: 1115, the LCDR1 comprises the amino acid sequence of SEQ ID NO: 1116, the LCDR2 comprises the amino acid sequence of SEQ ID NO: 1117, and the LCDR3 comprises the amino acid sequence of SEQ ID NO: 1118.

In some specific embodiments, in the antibody or antigen binding fragment provided herein, the HCDR1 comprises the amino acid sequence of SEQ ID NO: 1119, the HCDR2 comprises the amino acid sequence of SEQ ID NO: 1120, the HCDR3 comprises the amino acid sequence of SEQ ID NO: 1121, the LCDR1 comprises the amino acid sequence of SEQ ID NO: 1122, the LCDR2 comprises the amino acid sequence of SEQ ID NO: 1123, and the LCDR3 comprises the amino acid sequence of SEQ ID NO: 1124.

In some specific embodiments, in the antibody or antigen binding fragment provided herein, the HCDR1 comprises the amino acid sequence of SEQ ID NO: 1125, the HCDR2 comprises the amino acid sequence of SEQ ID NO: 1126, the HCDR3 comprises the amino acid sequence of SEQ ID NO: 1127, the LCDR1 comprises the amino acid sequence of SEQ ID NO: 1128, the LCDR2 comprises the amino acid sequence of SEQ ID NO: 1129, and the LCDR3 comprises the amino acid sequence of SEQ ID NO: 1130.

In some specific embodiments, in the antibody or antigen binding fragment provided herein, the HCDR1 comprises the amino acid sequence of SEQ ID NO: 855, the HCDR2 comprises the amino acid sequence of SEQ ID NO: 856, the HCDR3 comprises the amino acid sequence of SEQ ID NO: 857, the LCDR1 comprises the amino acid sequence of SEQ ID NO: 858, the LCDR2 comprises the amino acid sequence of SEQ ID NO: 1282, and the LCDR3 comprises the amino acid sequence of SEQ ID NO: 860.

In some embodiments, the antibody further comprises one or more framework regions of SEQ ID NOs: 401-494, and 1167-1178. In some embodiments, the antibody provided herein is a humanized antibody. Framework regions described herein are determined based upon the boundaries of the CDR numbering system. In other words, if the CDRs are determined by, e.g., Kabat, IMGT, or Chothia, then the framework regions are the amino acid residues surrounding the CDRs in the variable region in the format, from the N-terminus to C-terminus: FR1-CDR1-FR2-CDR2-FR3-CDR3-FR4. For example, FRI is defined as the amino acid residues N-terminal to the CDR1 amino acid residues as defined by, e.g., the Kabat numbering system, the IMGT numbering system, or the Chothia numbering system, FR2 is defined as the amino acid residues between CDR1 and CDR2 amino acid residues as defined by, e.g., the Kabat numbering system, the IMGT numbering system, or the Chothia numbering system, FR3 is defined as the amino acid residues between CDR2 and CDR3 amino acid residues as defined by, e.g., the Kabat numbering system, the IMGT numbering system, or the Chothia numbering system, and FR4 is defined as the amino acid residues C-terminal to the CDR3 amino acid residues as defined by, e.g., the Kabat numbering system, the IMGT numbering system, or the Chothia numbering system.

In some embodiments, the antibody or antigen binding fragment provided herein comprises a VH comprising the amino acid sequence of SEQ ID NO: 401. In some embodiments, the antibody or antigen binding fragment provided herein comprises a VL comprising the amino acid sequence of SEQ ID NO: 402. In some embodiments, the antibody or antigen binding fragment provided herein comprises a VH comprising the amino acid sequence of SEQ ID NO: 401, and a VL comprising the amino acid sequence of SEQ ID NO: 402.

In some embodiments, the antibody or antigen binding fragment provided herein comprises a VH comprising the amino acid sequence of SEQ ID NO: 403. In some embodiments, the antibody or antigen binding fragment provided herein comprises a VL comprising the amino acid sequence of SEQ ID NO: 404. In some embodiments, the antibody or antigen binding fragment provided herein comprises a VH comprising the amino acid sequence of SEQ ID NO: 403, and a VL comprising the amino acid sequence of SEQ ID NO: 404.

In some embodiments, the antibody or antigen binding fragment provided herein comprises a VH comprising the amino acid sequence of SEQ ID NO: 405. In some embodiments, the antibody or antigen binding fragment provided herein comprises a VL comprising the amino acid sequence of SEQ ID NO: 406. In some embodiments, the antibody or antigen binding fragment provided herein comprises a VH comprising the amino acid sequence of SEQ ID NO: 405, and a VL comprising the amino acid sequence of SEQ ID NO: 406.

In some embodiments, the antibody or antigen binding fragment provided herein comprises a VH comprising the amino acid sequence of SEQ ID NO: 407. In some embodiments, the antibody or antigen binding fragment provided herein comprises a VL comprising the amino acid sequence of SEQ ID NO: 408. In some embodiments, the antibody or antigen binding fragment provided herein comprises a VH comprising the amino acid sequence of SEQ ID NO: 407, and a VL comprising the amino acid sequence of SEQ ID NO: 408.

In some embodiments, the antibody or antigen binding fragment provided herein comprises a VH comprising the amino acid sequence of SEQ ID NO: 409. In some embodiments, the antibody or antigen binding fragment provided herein comprises a VL comprising the amino acid sequence of SEQ ID NO: 410. In some embodiments, the antibody or antigen binding fragment provided herein comprises a VH comprising the amino acid sequence of SEQ ID NO: 409, and a VL comprising the amino acid sequence of SEQ ID NO: 410.

In some embodiments, the antibody or antigen binding fragment provided herein comprises a VH comprising the amino acid sequence of SEQ ID NO: 411. In some embodiments, the antibody or antigen binding fragment provided herein comprises a VL comprising the amino acid sequence of SEQ ID NO: 412. In some embodiments, the antibody or antigen binding fragment provided herein comprises a VH comprising the amino acid sequence of SEQ ID NO: 411, and a VL comprising the amino acid sequence of SEQ ID NO: 412.

In some embodiments, the antibody or antigen binding fragment provided herein comprises a VH comprising the amino acid sequence of SEQ ID NO: 413. In some embodiments, the antibody or antigen binding fragment provided herein comprises a VL comprising the amino acid sequence of SEQ ID NO: 414. In some embodiments, the antibody or antigen binding fragment provided herein comprises a VH comprising the amino acid sequence of SEQ ID NO: 413, and a VL comprising the amino acid sequence of SEQ ID NO: 414.

In some embodiments, the antibody or antigen binding fragment provided herein comprises a VH comprising the amino acid sequence of SEQ ID NO: 415. In some embodiments, the antibody or antigen binding fragment provided herein comprises a VL comprising the amino acid sequence of SEQ ID NO: 416. In some embodiments, the antibody or antigen binding fragment provided herein comprises a VH comprising the amino acid sequence of SEQ ID NO: 415, and a VL comprising the amino acid sequence of SEQ ID NO: 416.

In some embodiments, the antibody or antigen binding fragment provided herein comprises a VH comprising the amino acid sequence of SEQ ID NO: 417. In some embodiments, the antibody or antigen binding fragment provided herein comprises a VL comprising the amino acid sequence of SEQ ID NO: 418. In some embodiments, the antibody or antigen binding fragment provided herein comprises a VH comprising the amino acid sequence of SEQ ID NO: 417, and a VL comprising the amino acid sequence of SEQ ID NO: 418.

In some embodiments, the antibody or antigen binding fragment provided herein comprises a VH comprising the amino acid sequence of SEQ ID NO: 419. In some embodiments, the antibody or antigen binding fragment provided herein comprises a VL comprising the amino acid sequence of SEQ ID NO: 420. In some embodiments, the antibody or antigen binding fragment provided herein comprises a VH comprising the amino acid sequence of SEQ ID NO: 419, and a VL comprising the amino acid sequence of SEQ ID NO: 420.

In some embodiments, the antibody or antigen binding fragment provided herein comprises a VH comprising the amino acid sequence of SEQ ID NO: 421. In some embodiments, the antibody or antigen binding fragment provided herein comprises a VL comprising the amino acid sequence of SEQ ID NO: 422. In some embodiments, the antibody or antigen binding fragment provided herein comprises a VH comprising the amino acid sequence of SEQ ID NO: 421, and a VL comprising the amino acid sequence of SEQ ID NO: 422.

In some embodiments, the antibody or antigen binding fragment provided herein comprises a VH comprising the amino acid sequence of SEQ ID NO: 423. In some embodiments, the antibody or antigen binding fragment provided herein comprises a VL comprising the amino acid sequence of SEQ ID NO: 424. In some embodiments, the antibody or antigen binding fragment provided herein comprises a VH comprising the amino acid sequence of SEQ ID NO: 423, and a VL comprising the amino acid sequence of SEQ ID NO: 424.

In some embodiments, the antibody or antigen binding fragment provided herein comprises a VH comprising the amino acid sequence of SEQ ID NO: 425. In some embodiments, the antibody or antigen binding fragment provided herein comprises a VL comprising the amino acid sequence of SEQ ID NO: 426. In some embodiments, the antibody or antigen binding fragment provided herein comprises a VH comprising the amino acid sequence of SEQ ID NO: 425, and a VL comprising the amino acid sequence of SEQ ID NO: 426.

In some embodiments, the antibody or antigen binding fragment provided herein comprises a VH comprising the amino acid sequence of SEQ ID NO: 427. In some embodiments, the antibody or antigen binding fragment provided herein comprises a VL comprising the amino acid sequence of SEQ ID NO: 428. In some embodiments, the antibody or antigen binding fragment provided herein comprises a VH comprising the amino acid sequence of SEQ ID NO: 427, and a VL comprising the amino acid sequence of SEQ ID NO: 428.

In some embodiments, the antibody or antigen binding fragment provided herein comprises a VH comprising the amino acid sequence of SEQ ID NO: 429. In some embodiments, the antibody or antigen binding fragment provided herein comprises a VL comprising the amino acid sequence of SEQ ID NO: 430. In some embodiments, the antibody or antigen binding fragment provided herein comprises a VH comprising the amino acid sequence of SEQ ID NO: 429, and a VL comprising the amino acid sequence of SEQ ID NO: 430.

In some embodiments, the antibody or antigen binding fragment provided herein comprises a VH comprising the amino acid sequence of SEQ ID NO: 431. In some embodiments, the antibody or antigen binding fragment provided herein comprises a VL comprising the amino acid sequence of SEQ ID NO: 432. In some embodiments, the antibody or antigen binding fragment provided herein comprises a VH comprising the amino acid sequence of SEQ ID NO: 431, and a VL comprising the amino acid sequence of SEQ ID NO: 432.

In some embodiments, the antibody or antigen binding fragment provided herein comprises a VH comprising the amino acid sequence of SEQ ID NO: 433. In some embodiments, the antibody or antigen binding fragment provided herein comprises a VL comprising the amino acid sequence of SEQ ID NO: 434. In some embodiments, the antibody or antigen binding fragment provided herein comprises a VH comprising the amino acid sequence of SEQ ID NO: 433, and a VL comprising the amino acid sequence of SEQ ID NO: 434.

In some embodiments, the antibody or antigen binding fragment provided herein comprises a VH comprising the amino acid sequence of SEQ ID NO: 435. In some embodiments, the antibody or antigen binding fragment provided herein comprises a VL comprising the amino acid sequence of SEQ ID NO: 436. In some embodiments, the antibody or antigen binding fragment provided herein comprises a VH comprising the amino acid sequence of SEQ ID NO: 435, and a VL comprising the amino acid sequence of SEQ ID NO: 436.

In some embodiments, the antibody or antigen binding fragment provided herein comprises a VH comprising the amino acid sequence of SEQ ID NO: 437. In some embodiments, the antibody or antigen binding fragment provided herein comprises a VL comprising the amino acid sequence of SEQ ID NO: 438. In some embodiments, the antibody or antigen binding fragment provided herein comprises a VH comprising the amino acid sequence of SEQ ID NO: 437, and a VL comprising the amino acid sequence of SEQ ID NO: 438.

In some embodiments, the antibody or antigen binding fragment provided herein comprises a VH comprising the amino acid sequence of SEQ ID NO: 439. In some embodiments, the antibody or antigen binding fragment provided herein comprises a VL comprising the amino acid sequence of SEQ ID NO: 440. In some embodiments, the antibody or antigen binding fragment provided herein comprises a VH comprising the amino acid sequence of SEQ ID NO: 439, and a VL comprising the amino acid sequence of SEQ ID NO: 440.

In some embodiments, the antibody or antigen binding fragment provided herein comprises a VH comprising the amino acid sequence of SEQ ID NO: 441. In some embodiments, the antibody or antigen binding fragment provided herein comprises a VL comprising the amino acid sequence of SEQ ID NO: 442. In some embodiments, the antibody or antigen binding fragment provided herein comprises a VH comprising the amino acid sequence of SEQ ID NO: 441, and a VL comprising the amino acid sequence of SEQ ID NO: 442.

In some embodiments, the antibody or antigen binding fragment provided herein comprises a VH comprising the amino acid sequence of SEQ ID NO: 443. In some embodiments, the antibody or antigen binding fragment provided herein comprises a VL comprising the amino acid sequence of SEQ ID NO: 444. In some embodiments, the antibody or antigen binding fragment provided herein comprises a VH comprising the amino acid sequence of SEQ ID NO: 443, and a VL comprising the amino acid sequence of SEQ ID NO: 444.

In some embodiments, the antibody or antigen binding fragment provided herein comprises a VH comprising the amino acid sequence of SEQ ID NO: 445. In some embodiments, the antibody or antigen binding fragment provided herein comprises a VL comprising the amino acid sequence of SEQ ID NO: 446. In some embodiments, the antibody or antigen binding fragment provided herein comprises a VH comprising the amino acid sequence of SEQ ID NO: 445, and a VL comprising the amino acid sequence of SEQ ID NO: 446.

In some embodiments, the antibody or antigen binding fragment provided herein comprises a VH comprising the amino acid sequence of SEQ ID NO: 447. In some embodiments, the antibody or antigen binding fragment provided herein comprises a VL comprising the amino acid sequence of SEQ ID NO: 448. In some embodiments, the antibody or antigen binding fragment provided herein comprises a VH comprising the amino acid sequence of SEQ ID NO: 447, and a VL comprising the amino acid sequence of SEQ ID NO: 448.

In some embodiments, the antibody or antigen binding fragment provided herein comprises a VH comprising the amino acid sequence of SEQ ID NO: 449. In some embodiments, the antibody or antigen binding fragment provided herein comprises a VL comprising the amino acid sequence of SEQ ID NO: 450. In some embodiments, the antibody or antigen binding fragment provided herein comprises a VH comprising the amino acid sequence of SEQ ID NO: 449, and a VL comprising the amino acid sequence of SEQ ID NO: 450.

In some embodiments, the antibody or antigen binding fragment provided herein comprises a VH comprising the amino acid sequence of SEQ ID NO: 451. In some embodiments, the antibody or antigen binding fragment provided herein comprises a VL comprising the amino acid sequence of SEQ ID NO: 452. In some embodiments, the antibody or antigen binding fragment provided herein comprises a VH comprising the amino acid sequence of SEQ ID NO: 451, and a VL comprising the amino acid sequence of SEQ ID NO: 452.

In some embodiments, the antibody or antigen binding fragment provided herein comprises a VH comprising the amino acid sequence of SEQ ID NO: 453. In some embodiments, the antibody or antigen binding fragment provided herein comprises a VL comprising the amino acid sequence of SEQ ID NO: 454. In some embodiments, the antibody or antigen binding fragment provided herein comprises a VH comprising the amino acid sequence of SEQ ID NO: 453, and a VL comprising the amino acid sequence of SEQ ID NO: 454.

In some embodiments, the antibody or antigen binding fragment provided herein comprises a VH comprising the amino acid sequence of SEQ ID NO: 455. In some embodiments, the antibody or antigen binding fragment provided herein comprises a VL comprising the amino acid sequence of SEQ ID NO: 456. In some embodiments, the antibody or antigen binding fragment provided herein comprises a VH comprising the amino acid sequence of SEQ ID NO: 455, and a VL comprising the amino acid sequence of SEQ ID NO: 456.

In some embodiments, the antibody or antigen binding fragment provided herein comprises a VH comprising the amino acid sequence of SEQ ID NO: 457. In some embodiments, the antibody or antigen binding fragment provided herein comprises a VL comprising the amino acid sequence of SEQ ID NO: 458. In some embodiments, the antibody or antigen binding fragment provided herein comprises a VH comprising the amino acid sequence of SEQ ID NO: 457, and a VL comprising the amino acid sequence of SEQ ID NO: 458.

In some embodiments, the antibody or antigen binding fragment provided herein comprises a VH comprising the amino acid sequence of SEQ ID NO: 459. In some embodiments, the antibody or antigen binding fragment provided herein comprises a VL comprising the amino acid sequence of SEQ ID NO: 460. In some embodiments, the antibody or antigen binding fragment provided herein comprises a VH comprising the amino acid sequence of SEQ ID NO: 459, and a VL comprising the amino acid sequence of SEQ ID NO: 460.

In some embodiments, the antibody or antigen binding fragment provided herein comprises a VH comprising the amino acid sequence of SEQ ID NO: 461. In some embodiments, the antibody or antigen binding fragment provided herein comprises a VL comprising the amino acid sequence of SEQ ID NO: 462. In some embodiments, the antibody or antigen binding fragment provided herein comprises a VH comprising the amino acid sequence of SEQ ID NO: 461, and a VL comprising the amino acid sequence of SEQ ID NO: 462.

In some embodiments, the antibody or antigen binding fragment provided herein comprises a VH comprising the amino acid sequence of SEQ ID NO: 463. In some embodiments, the antibody or antigen binding fragment provided herein comprises a VL comprising the amino acid sequence of SEQ ID NO: 464. In some embodiments, the antibody or antigen binding fragment provided herein comprises a VH comprising the amino acid sequence of SEQ ID NO: 463, and a VL comprising the amino acid sequence of SEQ ID NO: 464.

In some embodiments, the antibody or antigen binding fragment provided herein comprises a VH comprising the amino acid sequence of SEQ ID NO: 465. In some embodiments, the antibody or antigen binding fragment provided herein comprises a VL comprising the amino acid sequence of SEQ ID NO: 466. In some embodiments, the antibody or antigen binding fragment provided herein comprises a VH comprising the amino acid sequence of SEQ ID NO: 465, and a VL comprising the amino acid sequence of SEQ ID NO: 466.

In some embodiments, the antibody or antigen binding fragment provided herein comprises a VH comprising the amino acid sequence of SEQ ID NO: 467. In some embodiments, the antibody or antigen binding fragment provided herein comprises a VL comprising the amino acid sequence of SEQ ID NO: 468. In some embodiments, the antibody or antigen binding fragment provided herein comprises a VH comprising the amino acid sequence of SEQ ID NO: 467, and a VL comprising the amino acid sequence of SEQ ID NO: 468.

In some embodiments, the antibody or antigen binding fragment provided herein comprises a VH comprising the amino acid sequence of SEQ ID NO: 469. In some embodiments, the antibody or antigen binding fragment provided herein comprises a VL comprising the amino acid sequence of SEQ ID NO: 470. In some embodiments, the antibody or antigen binding fragment provided herein comprises a VH comprising the amino acid sequence of SEQ ID NO: 469, and a VL comprising the amino acid sequence of SEQ ID NO: 470.

In some embodiments, the antibody or antigen binding fragment provided herein comprises a VH comprising the amino acid sequence of SEQ ID NO: 471. In some embodiments, the antibody or antigen binding fragment provided herein comprises a VL comprising the amino acid sequence of SEQ ID NO: 472. In some embodiments, the antibody or antigen binding fragment provided herein comprises a VH comprising the amino acid sequence of SEQ ID NO: 471, and a VL comprising the amino acid sequence of SEQ ID NO: 472.

In some embodiments, the antibody or antigen binding fragment provided herein comprises a VH comprising the amino acid sequence of SEQ ID NO: 473. In some embodiments, the antibody or antigen binding fragment provided herein comprises a VL comprising the amino acid sequence of SEQ ID NO: 474. In some embodiments, the antibody or antigen binding fragment provided herein comprises a VH comprising the amino acid sequence of SEQ ID NO: 473, and a VL comprising the amino acid sequence of SEQ ID NO: 474.

In some embodiments, the antibody or antigen binding fragment provided herein comprises a VH comprising the amino acid sequence of SEQ ID NO: 475. In some embodiments, the antibody or antigen binding fragment provided herein comprises a VL comprising the amino acid sequence of SEQ ID NO: 476. In some embodiments, the antibody or antigen binding fragment provided herein comprises a VH comprising the amino acid sequence of SEQ ID NO: 475, and a VL comprising the amino acid sequence of SEQ ID NO: 476.

In some embodiments, the antibody or antigen binding fragment provided herein comprises a VH comprising the amino acid sequence of SEQ ID NO: 477. In some embodiments, the antibody or antigen binding fragment provided herein comprises a VL comprising the amino acid sequence of SEQ ID NO: 478. In some embodiments, the antibody or antigen binding fragment provided herein comprises a VH comprising the amino acid sequence of SEQ ID NO: 477, and a VL comprising the amino acid sequence of SEQ ID NO: 478.

In some embodiments, the antibody or antigen binding fragment provided herein comprises a VH comprising the amino acid sequence of SEQ ID NO: 479. In some embodiments, the antibody or antigen binding fragment provided herein comprises a VL comprising the amino acid sequence of SEQ ID NO: 480. In some embodiments, the antibody or antigen binding fragment provided herein comprises a VH comprising the amino acid sequence of SEQ ID NO: 479, and a VL comprising the amino acid sequence of SEQ ID NO: 480.

In some embodiments, the antibody or antigen binding fragment provided herein comprises a VH comprising the amino acid sequence of SEQ ID NO: 481. In some embodiments, the antibody or antigen binding fragment provided herein comprises a VL comprising the amino acid sequence of SEQ ID NO: 482. In some embodiments, the antibody or antigen binding fragment provided herein comprises a VH comprising the amino acid sequence of SEQ ID NO: 481, and a VL comprising the amino acid sequence of SEQ ID NO: 482.

In some embodiments, the antibody or antigen binding fragment provided herein comprises a VH comprising the amino acid sequence of SEQ ID NO: 483. In some embodiments, the antibody or antigen binding fragment provided herein comprises a VL comprising the amino acid sequence of SEQ ID NO: 484. In some embodiments, the antibody or antigen binding fragment provided herein comprises a VH comprising the amino acid sequence of SEQ ID NO: 483, and a VL comprising the amino acid sequence of SEQ ID NO: 484.

In some embodiments, the antibody or antigen binding fragment provided herein comprises a VH comprising the amino acid sequence of SEQ ID NO: 485. In some embodiments, the antibody or antigen binding fragment provided herein comprises a VL comprising the amino acid sequence of SEQ ID NO: 486. In some embodiments, the antibody or antigen binding fragment provided herein comprises a VH comprising the amino acid sequence of SEQ ID NO: 485, and a VL comprising the amino acid sequence of SEQ ID NO: 486.

In some embodiments, the antibody or antigen binding fragment provided herein comprises a VH comprising the amino acid sequence of SEQ ID NO: 487. In some embodiments, the antibody or antigen binding fragment provided herein comprises a VL comprising the amino acid sequence of SEQ ID NO: 488. In some embodiments, the antibody or antigen binding fragment provided herein comprises a VH comprising the amino acid sequence of SEQ ID NO: 487, and a VL comprising the amino acid sequence of SEQ ID NO: 488.

In some embodiments, the antibody or antigen binding fragment provided herein comprises a VH comprising the amino acid sequence of SEQ ID NO: 489. In some embodiments, the antibody or antigen binding fragment provided herein comprises a VL comprising the amino acid sequence of SEQ ID NO: 490. In some embodiments, the antibody or antigen binding fragment provided herein comprises a VH comprising the amino acid sequence of SEQ ID NO: 489, and a VL comprising the amino acid sequence of SEQ ID NO: 490.

In some embodiments, the antibody or antigen binding fragment provided herein comprises a VH comprising the amino acid sequence of SEQ ID NO: 491. In some embodiments, the antibody or antigen binding fragment provided herein comprises a VL comprising the amino acid sequence of SEQ ID NO: 492. In some embodiments, the antibody or antigen binding fragment provided herein comprises a VH comprising the amino acid sequence of SEQ ID NO: 491, and a VL comprising the amino acid sequence of SEQ ID NO: 492.

In some embodiments, the antibody or antigen binding fragment provided herein comprises a VH comprising the amino acid sequence of SEQ ID NO: 493. In some embodiments, the antibody or antigen binding fragment provided herein comprises a VL comprising the amino acid sequence of SEQ ID NO: 494. In some embodiments, the antibody or antigen binding fragment provided herein comprises a VH comprising the amino acid sequence of SEQ ID NO: 493, and a VL comprising the amino acid sequence of SEQ ID NO: 494.

In some embodiments, the antibody or antigen binding fragment provided herein comprises a VH comprising the amino acid sequence of SEQ ID NO: 1167. In some embodiments, the antibody or antigen binding fragment provided herein comprises a VL comprising the amino acid sequence of SEQ ID NO: 1168. In some embodiments, the antibody or antigen binding fragment provided herein comprises a VH comprising the amino acid sequence of SEQ ID NO: 1167, and a VL comprising the amino acid sequence of SEQ ID NO: 1168.

In some embodiments, the antibody or antigen binding fragment provided herein comprises a VH comprising the amino acid sequence of SEQ ID NO: 1169. In some embodiments, the antibody or antigen binding fragment provided herein comprises a VL comprising the amino acid sequence of SEQ ID NO: 1170. In some embodiments, the antibody or antigen binding fragment provided herein comprises a VH comprising the amino acid sequence of SEQ ID NO: 1169, and a VL comprising the amino acid sequence of SEQ ID NO: 1170.

In some embodiments, the antibody or antigen binding fragment provided herein comprises a VH comprising the amino acid sequence of SEQ ID NO: 1171. In some embodiments, the antibody or antigen binding fragment provided herein comprises a VL comprising the amino acid sequence of SEQ ID NO: 1172. In some embodiments, the antibody or antigen binding fragment provided herein comprises a VH comprising the amino acid sequence of SEQ ID NO: 1171, and a VL comprising the amino acid sequence of SEQ ID NO: 1172.

In some embodiments, the antibody or antigen binding fragment provided herein comprises a VH comprising the amino acid sequence of SEQ ID NO: 1173. In some embodiments, the antibody or antigen binding fragment provided herein comprises a VL comprising the amino acid sequence of SEQ ID NO: 1174. In some embodiments, the antibody or antigen binding fragment provided herein comprises a VH comprising the amino acid sequence of SEQ ID NO: 1173, and a VL comprising the amino acid sequence of SEQ ID NO: 1174.

In some embodiments, the antibody or antigen binding fragment provided herein comprises a VH comprising the amino acid sequence of SEQ ID NO: 1175. In some embodiments, the antibody or antigen binding fragment provided herein a VL comprising the amino acid sequence of SEQ ID NO: 1176. In some embodiments, the antibody or antigen binding fragment provided herein comprises a VH comprising the amino acid sequence of SEQ ID NO: 1175, and a VL comprising the amino acid sequence of SEQ ID NO: 1176.

In some embodiments, the antibody or antigen binding fragment provided herein comprises a VH comprising the amino acid sequence of SEQ ID NO: 1177. In some embodiments, the antibody or antigen binding fragment provided herein comprises a VL comprising the amino acid sequence of SEQ ID NO: 1178. In some embodiments, the antibody or antigen binding fragment provided herein comprises a VH comprising the amino acid sequence of SEQ ID NO: 1177, and a VL comprising the amino acid sequence of SEQ ID NO: 1178.

In certain embodiments, an antibody described herein or an antigen binding fragment thereof comprises amino acid sequences with certain percent identity relative to any antibody provided herein, for example, those described in Section 7 below.

In certain embodiments, the antibody described herein or an antigen binding fragment thereof comprises amino acid sequences with certain percent identity (such as at least about 80%, or at least about 81%, or at least about 82%, or at least about 83%, or at least about 84%, or at least about 85%, or at least about 86%, or at least about 87%, or at least about 88%, or at least about 89%, or as at least about 90%, or at least about 91%, or at least about 92%, or at least about 93%, or at least about 94%, or at least about 95%, or at least about 96%, or at least about 97%, or at least about 98%, or at least about 99%, or higher) relative to any antibody or fragment thereof provided herein, for example, a CDR, VH or VL in Tables 5-6, and 14-15. In some embodiments, the antibody described herein or an antigen binding fragment thereof comprises CDRs of any antibody or fragment thereof provided herein, for example as described in Tables 5-6, and 14-15.

The determination of percent identity between two sequences (e.g., amino acid sequences or nucleic acid sequences) can be accomplished as described in section 5.2.1 above.

In some embodiments, the antibody described herein or an antigen binding fragment thereof provided herein contains substitutions (e.g., conservative substitutions), insertions, or deletions relative to the reference sequence, but the antibody comprising that sequence retains the ability to bind to LILRB2. In some embodiments, a total of 1 to 10 amino acids have been substituted, inserted and/or deleted in a reference amino acid sequence. In some embodiments, substitutions, insertions, or deletions occur in regions outside the CDRs (i.e., in the FRs and/or constant regions).

In some embodiments, the position of one or more CDRs along the VH (e.g., CDR1, CDR2, or CDR3) and/or VL (e.g., CDR1, CDR2, or CDR3) region of an LILRB2 binding domain described herein may vary by one, two, three, four, five, or six amino acid positions so long as binding to LILRB2 (e.g., human LILRB2) is maintained (e.g., substantially maintained, for example, at least 50%, at least 60%, at least 70%, at least 80%, at least 90%, or at least 95%). For example, in some embodiments, the position defining a CDR of any of Tables 5-6, and 14-15 may vary by shifting the N-terminal and/or C-terminal boundary of the CDR by one, two, three, four, five, or six amino acids, relative to the current CDR position, so long as binding to LILRB2 (e.g., human LILRB2) is maintained (e.g., substantially maintained, for example, at least 50%, at least 60%, at least 70%, at least 80%, at least 90%, or at least 95%). Additionally or alternatively, in some embodiments, the length of one or more CDRs along the VH (e.g., CDR1, CDR2, or CDR3) and/or VL (e.g., CDR1, CDR2, or CDR3) region of an LILRB2 binding domain described herein may vary (e.g., be shorter or longer) by one, two, three, four, five, or more amino acids, so long as binding to LILRB2 (e.g., human LILRB2) is maintained (e.g., substantially maintained, for example, at least 50%, at least 60%, at least 70%, at least 80%, at least 90%, or at least 95%). For example, in some embodiments, a VH and/or VL CDR1, CDR2, and/or CDR3 described herein may be one, two, three, four, five or more amino acids shorter than one or more of the CDRs described by SEQ ID NOS: 849-1130, and 1282 so long as binding to LILRB2 (e.g., human LILRB2) is maintained (e.g., substantially maintained, for example, at least 50%, at least 60%, at least 70%, at least 80%, at least 90%, or at least 95%). In other embodiments, a VH and/or VL CDR1, CDR2, and/or CDR3 described herein may be one, two, three, four, five or more amino acids longer than one or more of the CDRs described by SEQ ID NOS: 849-1130, and 1282 so long as binding to LILRB2 (e.g., human LILRB2) is maintained (e.g., substantially maintained, for example, at least 50%, at least 60%, at least 70%, at least 80%, at least 90%, or at least 95%). In some embodiments, the amino terminus of a VH and/or VL CDR1, CDR2, and/or CDR3 described herein may be extended or shortened by one, two, three, four, five or more amino acids compared to one or more of the CDRs described by SEQ ID NOS: 849-1130, and 1282 so long as binding to LILRB2 (e.g., human LILRB2) is maintained (e.g., substantially maintained, for example, at least 50%, at least 60%, at least 70%, at least 80%, at least 90%, or at least 95%). Additionally or alternatively, in some embodiments, the carboxy terminus of a VH and/or VL CDR1, CDR2, and/or CDR3 described herein may be extended or shortened by one, two, three, four, five or more amino acids compared to one or more of the CDRs described by SEQ ID NOS: 849-1130, and 1282 so long as binding to LILRB2 (e.g., human LILRB2) is maintained (e.g., substantially maintained, for example, at least 50%, at least 60%, at least 70%, at least 80%, at least 90%, or at least 95%). Any method known in the art can be used to ascertain whether binding to LILRB2 (e.g., human LILRB2) is maintained, for example, the binding assays and conditions described in the “Examples” section described herein.

In other embodiments, the antibody described herein or an antigen binding fragment thereof further comprise conservative sequence modifications (e.g., in an LILRB2 binding domain). Conservative sequence modifications include conservative amino acid substitutions that include ones in which the amino acid residue is replaced with an amino acid residue having a similar side chain. Families of amino acid residues having similar side chains have been defined in the art. Thus, in some embodiments, a predicted nonessential amino acid residue in an LILRB2 is replaced with another amino acid residue from the same side chain family. Methods of identifying amino acid conservative substitutions which do not eliminate antigen binding and nucleotides encoding thereof are well-known in the art (see, e.g., Brummell et al., Biochem. 32:1180-1187 (1993); Kobayashi et al. Protein Eng. 12 (10): 879-884 (1999); and Burks et al. Proc. Natl. Acad. Sci. USA 94:412-417 (1997)). In some embodiments, the conservative sequence modifications described herein modify the amino acid sequences of the binding agents (e.g., antibodies), including human LILRB2 binding agents, by 50%, or 55%, or 60%, or 65%, or 70%, or 75%, or 80%, or 85%, or 90%, or 95%, or 98%, or 99%. In some embodiments, the amino acid sequence modifications refer to at most 1, 2, 3, 4, 5, or 6 amino acid substitutions to the CDRs, such as those described in any one of Tables 5-6, and 14-15. Thus, for example, each such CDR may contain up to 5 conservative amino acid substitutions, for example up to (not more than) 4 conservative amino acid substitutions, for example up to (not more than) 3 conservative amino acid substitutions, for example up to (not more than) 2 conservative amino acid substitutions, or no more than 1 conservative amino acid substitution. In some embodiments, the LILRB2 binding domain contains one or more, including six, CDRs having at least 75%, 80%, 85%, 90%, 95%, 96%, 97%, 98%, or 99% identity to the CDRs of any antibody or fragment thereof provided herein, for example in Tables 5-6, and 14-15.

In some embodiments, an LILRB2 binding domain contains a VH and a VL comprising CDRs identical to any antibody or fragment thereof provided herein, for example in Tables 5-6, and 14-15.

In some embodiments, the amino acid sequence modifications do not include any modification within an SDR. In some embodiments, the amino acid sequence modifications do not include any modification within a CDR (such as CDR1, CDR2, CDR3, or any combination thereof). In further embodiments, the amino acid sequence modifications are in the framework or constant region.

In some embodiments, the antibody provide herein contains substitutions (e.g., conservative substitutions), insertions, or deletions relative to the reference sequence, but the anti-LILRB2 antibody comprising that sequence retains the ability to bind to LILRB2. In some embodiments, a total of 1 to 10 amino acids have been substituted, inserted and/or deleted in a reference amino acid sequence. In some embodiments, substitutions, insertions, or deletions occur in regions outside the CDRs (i.e., in the FRs). Optionally, the anti-LILRB2 antibody provided herein includes post-translational modifications of a reference sequence.

In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 401, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 402. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 403, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 404. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 405, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 406. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 407, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 408. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 409, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 410. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 411, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 412. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 413, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 414. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 415, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 416. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 417, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 418. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 419, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 420. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 421, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 422. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 423, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 424. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 425, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 426. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 427, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 428. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 429, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 430. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 431, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 432. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 433, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 434. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 435, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 436. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 437, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 438. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 439, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 440. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 441, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 442. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 443, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 444. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 445, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 446. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 447, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 448. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 449, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 450. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 451, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 452. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 453, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 454. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 455, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 456. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 457, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 458. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 459, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 460. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 461, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 462. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 463, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 464. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 465, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 466. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 467, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 468. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 469, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 470. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 471, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 472. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 473, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 474. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 475, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 476. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 477, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 478. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 479, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 480. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 481, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 482. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 483, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 484. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 485, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 486. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 487, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 488. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 489, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 490. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 491, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 492. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 493, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 494. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 1167, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 1168. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 1169, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 1170. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 1171, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 1172. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 1173, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 1174. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 1175, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 1176. In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 1177, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 1178. In all the embodiments described above, the antibodies bind to LILRB2.

In some embodiments, functional epitopes can be mapped, e.g., by combinatorial alanine scanning, to identify amino acids in the LILRB2 protein that are necessary for interaction with anti-LILRB2 antibodies provided herein. In some embodiments, conformational and crystal structure of anti-LILRB2 antibody bound to LILRB2 may be employed to identify the epitopes. In some embodiments, the present disclosure provides an antibody that specifically binds to the same epitope as any of the anti-LILRB2 antibodies provided herein. For example, in some embodiments, the antibody or antigen binding fragment provided herein binds to the same epitope as an anti-LILRB2 antibody comprising a VH comprising the amino acid sequence of SEQ ID NO: 401, and a VL comprising the amino acid sequence of SEQ ID NO: 402. In some embodiments, the antibody or antigen binding fragment provided herein binds to the same epitope as an anti-LILRB2 antibody comprising a VH comprising the amino acid sequence of SEQ ID NO: 403, and a VL comprising the amino acid sequence of SEQ ID NO: 404. In some embodiments, the antibody or antigen binding fragment provided herein binds to the same epitope as an anti-LILRB2 antibody comprising a VH comprising the amino acid sequence of SEQ ID NO: 405, and a VL comprising the amino acid sequence of SEQ ID NO: 406. In some embodiments, the antibody or antigen binding fragment provided herein binds to the same epitope as an anti-LILRB2 antibody comprising a VH comprising the amino acid sequence of SEQ ID NO: 407, and a VL comprising the amino acid sequence of SEQ ID NO: 408. In some embodiments, the antibody or antigen binding fragment provided herein binds to the same epitope as an anti-LILRB2 antibody comprising a VH comprising the amino acid sequence of SEQ ID NO: 409, and a VL comprising the amino acid sequence of SEQ ID NO: 410. In some embodiments, the antibody or antigen binding fragment provided herein binds to the same epitope as an anti-LILRB2 antibody comprising a VH comprising the amino acid sequence of SEQ ID NO: 411, and a VL comprising the amino acid sequence of SEQ ID NO: 412. In some embodiments, the antibody or antigen binding fragment provided herein binds to the same epitope as an anti-LILRB2 antibody comprising a VH comprising the amino acid sequence of SEQ ID NO: 413, and a VL comprising the amino acid sequence of SEQ ID NO: 414. In some embodiments, the antibody or antigen binding fragment provided herein binds to the same epitope as an anti-LILRB2 antibody comprising a VH comprising the amino acid sequence of SEQ ID NO: 415, and a VL comprising the amino acid sequence of SEQ ID NO: 416. In some embodiments, the antibody or antigen binding fragment provided herein binds to the same epitope as an anti-LILRB2 antibody comprising a VH comprising the amino acid sequence of SEQ ID NO: 417, and a VL comprising the amino acid sequence of SEQ ID NO: 418. In some embodiments, the antibody or antigen binding fragment provided herein binds to the same epitope as an anti-LILRB2 antibody comprising a VH comprising the amino acid sequence of SEQ ID NO: 419, and a VL comprising the amino acid sequence of SEQ ID NO: 420. In some embodiments, the antibody or antigen binding fragment provided herein binds to the same epitope as an anti-LILRB2 antibody comprising a VH comprising the amino acid sequence of SEQ ID NO: 421, and a VL comprising the amino acid sequence of SEQ ID NO: 422. In some embodiments, the antibody or antigen binding fragment provided herein binds to the same epitope as an anti-LILRB2 antibody comprising a VH comprising the amino acid sequence of SEQ ID NO: 423, and a VL comprising the amino acid sequence of SEQ ID NO: 424. In some embodiments, the antibody or antigen binding fragment provided herein binds to the same epitope as an anti-LILRB2 antibody comprising a VH comprising the amino acid sequence of SEQ ID NO: 425, and a VL comprising the amino acid sequence of SEQ ID NO: 426. In some embodiments, the antibody or antigen binding fragment provided herein binds to the same epitope as an anti-LILRB2 antibody comprising a VH comprising the amino acid sequence of SEQ ID NO: 427, and a VL comprising the amino acid sequence of SEQ ID NO: 428. In some embodiments, the antibody or antigen binding fragment provided herein binds to the same epitope as an anti-LILRB2 antibody comprising a VH comprising the amino acid sequence of SEQ ID NO: 429, and a VL comprising the amino acid sequence of SEQ ID NO: 430. In some embodiments, the antibody or antigen binding fragment provided herein binds to the same epitope as an anti-LILRB2 antibody comprising a VH comprising the amino acid sequence of SEQ ID NO: 431, and a VL comprising the amino acid sequence of SEQ ID NO: 432. In some embodiments, the antibody or antigen binding fragment provided herein binds to the same epitope as an anti-LILRB2 antibody comprising a VH comprising the amino acid sequence of SEQ ID NO: 433, and a VL comprising the amino acid sequence of SEQ ID NO: 434. In some embodiments, the antibody or antigen binding fragment provided herein binds to the same epitope as an anti-LILRB2 antibody comprising a VH comprising the amino acid sequence of SEQ ID NO: 435, and a VL comprising the amino acid sequence of SEQ ID NO: 436. In some embodiments, the antibody or antigen binding fragment provided herein binds to the same epitope as an anti-LILRB2 antibody comprising a VH comprising the amino acid sequence of SEQ ID NO: 437, and a VL comprising the amino acid sequence of SEQ ID NO: 438. In some embodiments, the antibody or antigen binding fragment provided herein binds to the same epitope as an anti-LILRB2 antibody comprising a VH comprising the amino acid sequence of SEQ ID NO: 439, and a VL comprising the amino acid sequence of SEQ ID NO: 440. In some embodiments, the antibody or antigen binding fragment provided herein binds to the same epitope as an anti-LILRB2 antibody comprising a VH comprising the amino acid sequence of SEQ ID NO: 441, and a VL comprising the amino acid sequence of SEQ ID NO: 442. In some embodiments, the antibody or antigen binding fragment provided herein binds to the same epitope as an anti-LILRB2 antibody comprising a VH comprising the amino acid sequence of SEQ ID NO: 443, and a VL comprising the amino acid sequence of SEQ ID NO: 444. In some embodiments, the antibody or antigen binding fragment provided herein binds to the same epitope as an anti-LILRB2 antibody comprising a VH comprising the amino acid sequence of SEQ ID NO: 445, and a VL comprising the amino acid sequence of SEQ ID NO: 446. In some embodiments, the antibody or antigen binding fragment provided herein binds to the same epitope as an anti-LILRB2 antibody comprising a VH comprising the amino acid sequence of SEQ ID NO: 447, and a VL comprising the amino acid sequence of SEQ ID NO: 448. In some embodiments, the antibody or antigen binding fragment provided herein binds to the same epitope as an anti-LILRB2 antibody comprising a VH comprising the amino acid sequence of SEQ ID NO: 449, and a VL comprising the amino acid sequence of SEQ ID NO: 450. In some embodiments, the antibody or antigen binding fragment provided herein binds to the same epitope as an anti-LILRB2 antibody comprising a VH comprising the amino acid sequence of SEQ ID NO: 451, and a VL comprising the amino acid sequence of SEQ ID NO: 452. In some embodiments, the antibody or antigen binding fragment provided herein binds to the same epitope as an anti-LILRB2 antibody comprising a VH comprising the amino acid sequence of SEQ ID NO: 453, and a VL comprising the amino acid sequence of SEQ ID NO: 454. In some embodiments, the antibody or antigen binding fragment provided herein binds to the same epitope as an anti-LILRB2 antibody comprising a VH comprising the amino acid sequence of SEQ ID NO: 455, and a VL comprising the amino acid sequence of SEQ ID NO: 456. In some embodiments, the antibody or antigen binding fragment provided herein binds to the same epitope as an anti-LILRB2 antibody comprising a VH comprising the amino acid sequence of SEQ ID NO: 457, and a VL comprising the amino acid sequence of SEQ ID NO: 458. In some embodiments, the antibody or antigen binding fragment provided herein binds to the same epitope as an anti-LILRB2 antibody comprising a VH comprising the amino acid sequence of SEQ ID NO: 459, and a VL comprising the amino acid sequence of SEQ ID NO: 460. In some embodiments, the antibody or antigen binding fragment provided herein binds to the same epitope as an anti-LILRB2 antibody comprising a VH comprising the amino acid sequence of SEQ ID NO: 461, and a VL comprising the amino acid sequence of SEQ ID NO: 462. In some embodiments, the antibody or antigen binding fragment provided herein binds to the same epitope as an anti-LILRB2 antibody comprising a VH comprising the amino acid sequence of SEQ ID NO: 463, and a VL comprising the amino acid sequence of SEQ ID NO: 464. In some embodiments, the antibody or antigen binding fragment provided herein binds to the same epitope as an anti-LILRB2 antibody comprising a VH comprising the amino acid sequence of SEQ ID NO: 465, and a VL comprising the amino acid sequence of SEQ ID NO: 466. In some embodiments, the antibody or antigen binding fragment provided herein binds to the same epitope as an anti-LILRB2 antibody comprising a VH comprising the amino acid sequence of SEQ ID NO: 467, and a VL comprising the amino acid sequence of SEQ ID NO: 468. In some embodiments, the antibody or antigen binding fragment provided herein binds to the same epitope as an anti-LILRB2 antibody comprising a VH comprising the amino acid sequence of SEQ ID NO: 469, and a VL comprising the amino acid sequence of SEQ ID NO: 470. In some embodiments, the antibody or antigen binding fragment provided herein binds to the same epitope as an anti-LILRB2 antibody comprising a VH comprising the amino acid sequence of SEQ ID NO: 471, and a VL comprising the amino acid sequence of SEQ ID NO: 472. In some embodiments, the antibody or antigen binding fragment provided herein binds to the same epitope as an anti-LILRB2 antibody comprising a VH comprising the amino acid sequence of SEQ ID NO: 473, and a VL comprising the amino acid sequence of SEQ ID NO: 474. In some embodiments, the antibody or antigen binding fragment provided herein binds to the same epitope as an anti-LILRB2 antibody comprising a VH comprising the amino acid sequence of SEQ ID NO: 475, and a VL comprising the amino acid sequence of SEQ ID NO: 476. In some embodiments, the antibody or antigen binding fragment provided herein binds to the same epitope as an anti-LILRB2 antibody comprising a VH comprising the amino acid sequence of SEQ ID NO: 477, and a VL comprising the amino acid sequence of SEQ ID NO: 478. In some embodiments, the antibody or antigen binding fragment provided herein binds to the same epitope as an anti-LILRB2 antibody comprising a VH comprising the amino acid sequence of SEQ ID NO: 479, and a VL comprising the amino acid sequence of SEQ ID NO: 480. In some embodiments, the antibody or antigen binding fragment provided herein binds to the same epitope as an anti-LILRB2 antibody comprising a VH comprising the amino acid sequence of SEQ ID NO: 481, and a VL comprising the amino acid sequence of SEQ ID NO: 482. In some embodiments, the antibody or antigen binding fragment provided herein binds to the same epitope as an anti-LILRB2 antibody comprising a VH comprising the amino acid sequence of SEQ ID NO: 483, and a VL comprising the amino acid sequence of SEQ ID NO: 484. In some embodiments, the antibody or antigen binding fragment provided herein binds to the same epitope as an anti-LILRB2 antibody comprising a VH comprising the amino acid sequence of SEQ ID NO: 485, and a VL comprising the amino acid sequence of SEQ ID NO: 486. In some embodiments, the antibody or antigen binding fragment provided herein binds to the same epitope as an anti-LILRB2 antibody comprising a VH comprising the amino acid sequence of SEQ ID NO: 487, and a VL comprising the amino acid sequence of SEQ ID NO: 488. In some embodiments, the antibody or antigen binding fragment provided herein binds to the same epitope as an anti-LILRB2 antibody comprising a VH comprising the amino acid sequence of SEQ ID NO: 489, and a VL comprising the amino acid sequence of SEQ ID NO: 490. In some embodiments, the antibody or antigen binding fragment provided herein binds to the same epitope as an anti-LILRB2 antibody comprising a VH comprising the amino acid sequence of SEQ ID NO: 491, and a VL comprising the amino acid sequence of SEQ ID NO: 492. In some embodiments, the antibody or antigen binding fragment provided herein binds to the same epitope as an anti-LILRB2 antibody comprising a VH comprising the amino acid sequence of SEQ ID NO: 493, and a VL comprising the amino acid sequence of SEQ ID NO: 494. In some embodiments, the antibody or antigen binding fragment provided herein binds to the same epitope as an anti-LILRB2 antibody comprising a VH comprising the amino acid sequence of SEQ ID NO: 1167, and a VL comprising the amino acid sequence of SEQ ID NO: 1168. In some embodiments, the antibody or antigen binding fragment provided herein binds to the same epitope as an anti-LILRB2 antibody comprising a VH comprising the amino acid sequence of SEQ ID NO: 1169, and a VL comprising the amino acid sequence of SEQ ID NO: 1170. In some embodiments, the antibody or antigen binding fragment provided herein binds to the same epitope as an anti-LILRB2 antibody comprising a VH comprising the amino acid sequence of SEQ ID NO: 1171, and a VL comprising the amino acid sequence of SEQ ID NO: 1172. In some embodiments, the antibody or antigen binding fragment provided herein binds to the same epitope as an anti-LILRB2 antibody comprising a VH comprising the amino acid sequence of SEQ ID NO: 1173, and a VL comprising the amino acid sequence of SEQ ID NO: 1174. In some embodiments, the antibody or antigen binding fragment provided herein binds to the same epitope as an anti-LILRB2 antibody comprising a VH comprising the amino acid sequence of SEQ ID NO: 1175, and a VL comprising the amino acid sequence of SEQ ID NO: 1176. In some embodiments, the antibody or antigen binding fragment provided herein binds to the same epitope as an anti-LILRB2 antibody comprising a VH comprising the amino acid sequence of SEQ ID NO: 1177, and a VL comprising the amino acid sequence of SEQ ID NO: 1178.

In some embodiments, provided herein is an anti-LILRB2 antibody, or antigen binding fragment thereof, that specifically binds to LILRB2 competitively with any one of the anti-LILRB2 antibodies described herein. In some embodiments, the antibody or antigen binding fragment provided herein specifically binds to LILRB2 competitively with an anti-LILRB2 antibody comprising a VH comprising the amino acid sequence of SEQ ID NO: 401, and a VL comprising the amino acid sequence of SEQ ID NO: 402. In some embodiments, the antibody or antigen binding fragment provided herein specifically binds to LILRB2 competitively with an anti-LILRB2 antibody comprising a VH comprising the amino acid sequence of SEQ ID NO: 403, and a VL comprising the amino acid sequence of SEQ ID NO: 404. In some embodiments, the antibody or antigen binding fragment provided herein specifically binds to LILRB2 competitively with an anti-LILRB2 antibody comprising a VH comprising the amino acid sequence of SEQ ID NO: 405, and a VL comprising the amino acid sequence of SEQ ID NO: 406. In some embodiments, the antibody or antigen binding fragment provided herein specifically binds to LILRB2 competitively with an anti-LILRB2 antibody comprising a VH comprising the amino acid sequence of SEQ ID NO: 407, and a VL comprising the amino acid sequence of SEQ ID NO: 408. In some embodiments, the antibody or antigen binding fragment provided herein specifically binds to LILRB2 competitively with an anti-LILRB2 antibody comprising a VH comprising the amino acid sequence of SEQ ID NO: 409, and a VL comprising the amino acid sequence of SEQ ID NO: 410. In some embodiments, the antibody or antigen binding fragment provided herein specifically binds to LILRB2 competitively with an anti-LILRB2 antibody comprising a VH comprising the amino acid sequence of SEQ ID NO: 411, and a VL comprising the amino acid sequence of SEQ ID NO: 412. In some embodiments, the antibody or antigen binding fragment provided herein specifically binds to LILRB2 competitively with an anti-LILRB2 antibody comprising a VH comprising the amino acid sequence of SEQ ID NO: 413, and a VL comprising the amino acid sequence of SEQ ID NO: 414. In some embodiments, the antibody or antigen binding fragment provided herein specifically binds to LILRB2 competitively with an anti-LILRB2 antibody comprising a VH comprising the amino acid sequence of SEQ ID NO: 415, and a VL comprising the amino acid sequence of SEQ ID NO: 416. In some embodiments, the antibody or antigen binding fragment provided herein specifically binds to LILRB2 competitively with an anti-LILRB2 antibody comprising a VH comprising the amino acid sequence of SEQ ID NO: 417, and a VL comprising the amino acid sequence of SEQ ID NO: 418. In some embodiments, the antibody or antigen binding fragment provided herein specifically binds to LILRB2 competitively with an anti-LILRB2 antibody comprising a VH comprising the amino acid sequence of SEQ ID NO: 419, and a VL comprising the amino acid sequence of SEQ ID NO: 420. In some embodiments, the antibody or antigen binding fragment provided herein specifically binds to LILRB2 competitively with an anti-LILRB2 antibody comprising a VH comprising the amino acid sequence of SEQ ID NO: 421, and a VL comprising the amino acid sequence of SEQ ID NO: 422. In some embodiments, the antibody or antigen binding fragment provided herein specifically binds to LILRB2 competitively with an anti-LILRB2 antibody comprising a VH comprising the amino acid sequence of SEQ ID NO: 423, and a VL comprising the amino acid sequence of SEQ ID NO: 424. In some embodiments, the antibody or antigen binding fragment provided herein specifically binds to LILRB2 competitively with an anti-LILRB2 antibody comprising a VH comprising the amino acid sequence of SEQ ID NO: 425, and a VL comprising the amino acid sequence of SEQ ID NO: 426. In some embodiments, the antibody or antigen binding fragment provided herein specifically binds to LILRB2 competitively with an anti-LILRB2 antibody comprising a VH comprising the amino acid sequence of SEQ ID NO: 427, and a VL comprising the amino acid sequence of SEQ ID NO: 428. In some embodiments, the antibody or antigen binding fragment provided herein specifically binds to LILRB2 competitively with an anti-LILRB2 antibody comprising a VH comprising the amino acid sequence of SEQ ID NO: 429, and a VL comprising the amino acid sequence of SEQ ID NO: 430. In some embodiments, the antibody or antigen binding fragment provided herein specifically binds to LILRB2 competitively with an anti-LILRB2 antibody comprising a VH comprising the amino acid sequence of SEQ ID NO: 431, and a VL comprising the amino acid sequence of SEQ ID NO: 432. In some embodiments, the antibody or antigen binding fragment provided herein specifically binds to LILRB2 competitively with an anti-LILRB2 antibody comprising a VH comprising the amino acid sequence of SEQ ID NO: 433, and a VL comprising the amino acid sequence of SEQ ID NO: 434. In some embodiments, the antibody or antigen binding fragment provided herein specifically binds to LILRB2 competitively with an anti-LILRB2 antibody comprising a VH comprising the amino acid sequence of SEQ ID NO: 435, and a VL comprising the amino acid sequence of SEQ ID NO: 436. In some embodiments, the antibody or antigen binding fragment provided herein specifically binds to LILRB2 competitively with an anti-LILRB2 antibody comprising a VH comprising the amino acid sequence of SEQ ID NO: 437, and a VL comprising the amino acid sequence of SEQ ID NO: 438. In some embodiments, the antibody or antigen binding fragment provided herein specifically binds to LILRB2 competitively with an anti-LILRB2 antibody comprising a VH comprising the amino acid sequence of SEQ ID NO: 439, and a VL comprising the amino acid sequence of SEQ ID NO: 440. In some embodiments, the antibody or antigen binding fragment provided herein specifically binds to LILRB2 competitively with an anti-LILRB2 antibody comprising a VH comprising the amino acid sequence of SEQ ID NO: 441, and a VL comprising the amino acid sequence of SEQ ID NO: 442. In some embodiments, the antibody or antigen binding fragment provided herein specifically binds to LILRB2 competitively with an anti-LILRB2 antibody comprising a VH comprising the amino acid sequence of SEQ ID NO: 443, and a VL comprising the amino acid sequence of SEQ ID NO: 444. In some embodiments, the antibody or antigen binding fragment provided herein specifically binds to LILRB2 competitively with an anti-LILRB2 antibody comprising a VH comprising the amino acid sequence of SEQ ID NO: 445, and a VL comprising the amino acid sequence of SEQ ID NO: 446. In some embodiments, the antibody or antigen binding fragment provided herein specifically binds to LILRB2 competitively with an anti-LILRB2 antibody comprising a VH comprising the amino acid sequence of SEQ ID NO: 447, and a VL comprising the amino acid sequence of SEQ ID NO: 448. In some embodiments, the antibody or antigen binding fragment provided herein specifically binds to LILRB2 competitively with an anti-LILRB2 antibody comprising a VH comprising the amino acid sequence of SEQ ID NO: 449, and a VL comprising the amino acid sequence of SEQ ID NO: 450. In some embodiments, the antibody or antigen binding fragment provided herein specifically binds to LILRB2 competitively with an anti-LILRB2 antibody comprising a VH comprising the amino acid sequence of SEQ ID NO: 451, and a VL comprising the amino acid sequence of SEQ ID NO: 452. In some embodiments, the antibody or antigen binding fragment provided herein specifically binds to LILRB2 competitively with an anti-LILRB2 antibody comprising a VH comprising the amino acid sequence of SEQ ID NO: 453, and a VL comprising the amino acid sequence of SEQ ID NO: 454. In some embodiments, the antibody or antigen binding fragment provided herein specifically binds to LILRB2 competitively with an anti-LILRB2 antibody comprising a VH comprising the amino acid sequence of SEQ ID NO: 455, and a VL comprising the amino acid sequence of SEQ ID NO: 456. In some embodiments, the antibody or antigen binding fragment provided herein specifically binds to LILRB2 competitively with an anti-LILRB2 antibody comprising a VH comprising the amino acid sequence of SEQ ID NO: 457, and a VL comprising the amino acid sequence of SEQ ID NO: 458. In some embodiments, the antibody or antigen binding fragment provided herein specifically binds to LILRB2 competitively with an anti-LILRB2 antibody comprising a VH comprising the amino acid sequence of SEQ ID NO: 459, and a VL comprising the amino acid sequence of SEQ ID NO: 460. In some embodiments, the antibody or antigen binding fragment provided herein specifically binds to LILRB2 competitively with an anti-LILRB2 antibody comprising a VH comprising the amino acid sequence of SEQ ID NO: 461, and a VL comprising the amino acid sequence of SEQ ID NO: 462. In some embodiments, the antibody or antigen binding fragment provided herein specifically binds to LILRB2 competitively with an anti-LILRB2 antibody comprising a VH comprising the amino acid sequence of SEQ ID NO: 463, and a VL comprising the amino acid sequence of SEQ ID NO: 464. In some embodiments, the antibody or antigen binding fragment provided herein specifically binds to LILRB2 competitively with an anti-LILRB2 antibody comprising a VH comprising the amino acid sequence of SEQ ID NO: 465, and a VL comprising the amino acid sequence of SEQ ID NO: 466. In some embodiments, the antibody or antigen binding fragment provided herein specifically binds to LILRB2 competitively with an anti-LILRB2 antibody comprising a VH comprising the amino acid sequence of SEQ ID NO: 467, and a VL comprising the amino acid sequence of SEQ ID NO: 468. In some embodiments, the antibody or antigen binding fragment provided herein specifically binds to LILRB2 competitively with an anti-LILRB2 antibody comprising a VH comprising the amino acid sequence of SEQ ID NO: 469, and a VL comprising the amino acid sequence of SEQ ID NO: 470. In some embodiments, the antibody or antigen binding fragment provided herein specifically binds to LILRB2 competitively with an anti-LILRB2 antibody comprising a VH comprising the amino acid sequence of SEQ ID NO: 471, and a VL comprising the amino acid sequence of SEQ ID NO: 472. In some embodiments, the antibody or antigen binding fragment provided herein specifically binds to LILRB2 competitively with an anti-LILRB2 antibody comprising a VH comprising the amino acid sequence of SEQ ID NO: 473, and a VL comprising the amino acid sequence of SEQ ID NO: 474. In some embodiments, the antibody or antigen binding fragment provided herein specifically binds to LILRB2 competitively with an anti-LILRB2 antibody comprising a VH comprising the amino acid sequence of SEQ ID NO: 475, and a VL comprising the amino acid sequence of SEQ ID NO: 476. In some embodiments, the antibody or antigen binding fragment provided herein specifically binds to LILRB2 competitively with an anti-LILRB2 antibody comprising a VH comprising the amino acid sequence of SEQ ID NO: 477, and a VL comprising the amino acid sequence of SEQ ID NO: 478. In some embodiments, the antibody or antigen binding fragment provided herein specifically binds to LILRB2 competitively with an anti-LILRB2 antibody comprising a VH comprising the amino acid sequence of SEQ ID NO: 479, and a VL comprising the amino acid sequence of SEQ ID NO: 480. In some embodiments, the antibody or antigen binding fragment provided herein specifically binds to LILRB2 competitively with an anti-LILRB2 antibody comprising a VH comprising the amino acid sequence of SEQ ID NO: 481, and a VL comprising the amino acid sequence of SEQ ID NO: 482. In some embodiments, the antibody or antigen binding fragment provided herein specifically binds to LILRB2 competitively with an anti-LILRB2 antibody comprising a VH comprising the amino acid sequence of SEQ ID NO: 483, and a VL comprising the amino acid sequence of SEQ ID NO: 484. In some embodiments, the antibody or antigen binding fragment provided herein specifically binds to LILRB2 competitively with an anti-LILRB2 antibody comprising a VH comprising the amino acid sequence of SEQ ID NO: 485, and a VL comprising the amino acid sequence of SEQ ID NO: 486. In some embodiments, the antibody or antigen binding fragment provided herein specifically binds to LILRB2 competitively with an anti-LILRB2 antibody comprising a VH comprising the amino acid sequence of SEQ ID NO: 487, and a VL comprising the amino acid sequence of SEQ ID NO: 488. In some embodiments, the antibody or antigen binding fragment provided herein specifically binds to LILRB2 competitively with an anti-LILRB2 antibody comprising a VH comprising the amino acid sequence of SEQ ID NO: 489, and a VL comprising the amino acid sequence of SEQ ID NO: 490. In some embodiments, the antibody or antigen binding fragment provided herein specifically binds to LILRB2 competitively with an anti-LILRB2 antibody comprising a VH comprising the amino acid sequence of SEQ ID NO: 491, and a VL comprising the amino acid sequence of SEQ ID NO: 492. In some embodiments, the antibody or antigen binding fragment provided herein specifically binds to LILRB2 competitively with an anti-LILRB2 antibody comprising a VH comprising the amino acid sequence of SEQ ID NO: 493, and a VL comprising the amino acid sequence of SEQ ID NO: 494. In some embodiments, the antibody or antigen binding fragment provided herein specifically binds to LILRB2 competitively with an anti-LILRB2 antibody comprising a VH comprising the amino acid sequence of SEQ ID NO: 1167, and a VL comprising the amino acid sequence of SEQ ID NO: 1168. In some embodiments, the antibody or antigen binding fragment provided herein specifically binds to LILRB2 competitively with an anti-LILRB2 antibody comprising a VH comprising the amino acid sequence of SEQ ID NO: 1169, and a VL comprising the amino acid sequence of SEQ ID NO: 1170. In some embodiments, the antibody or antigen binding fragment provided herein specifically binds to LILRB2 competitively with an anti-LILRB2 antibody comprising a VH comprising the amino acid sequence of SEQ ID NO: 1171, and a VL comprising the amino acid sequence of SEQ ID NO: 1172. In some embodiments, the antibody or antigen binding fragment provided herein specifically binds to LILRB2 competitively with an anti-LILRB2 antibody comprising a VH comprising the amino acid sequence of SEQ ID NO: 1173, and a VL comprising the amino acid sequence of SEQ ID NO: 1174. In some embodiments, the antibody or antigen binding fragment provided herein specifically binds to LILRB2 competitively with an anti-LILRB2 antibody comprising a VH comprising the amino acid sequence of SEQ ID NO: 1175, and a VL comprising the amino acid sequence of SEQ ID NO: 1176. In some embodiments, the antibody or antigen binding fragment provided herein specifically binds to LILRB2 competitively with an anti-LILRB2 antibody comprising a VH comprising the amino acid sequence of SEQ ID NO: 1177, and a VL comprising the amino acid sequence of SEQ ID NO: 1178.

In some embodiments, the antibody or antigen binding fragment provided herein comprises a heavy chain comprising the amino acid sequence of SEQ ID NO: 189, and a light chain comprising the amino acid sequence of SEQ ID NO: 190. In some embodiments, the antibody or antigen binding fragment provided herein comprises a heavy chain comprising the amino acid sequence of SEQ ID NO: 191, and a light chain comprising the amino acid sequence of SEQ ID NO: 192. In some embodiments, the antibody or antigen binding fragment provided herein comprises a heavy chain comprising the amino acid sequence of SEQ ID NO: 193, and a light chain comprising the amino acid sequence of SEQ ID NO: 194. In some embodiments, the antibody or antigen binding fragment provided herein comprises a heavy chain comprising the amino acid sequence of SEQ ID NO: 195, and a light chain comprising the amino acid sequence of SEQ ID NO: 196. In some embodiments, the antibody or antigen binding fragment provided herein comprises a heavy chain comprising the amino acid sequence of SEQ ID NO: 197, and a light chain comprising the amino acid sequence of SEQ ID NO: 198. In some embodiments, the antibody or antigen binding fragment provided herein comprises a heavy chain comprising the amino acid sequence of SEQ ID NO: 199, and a light chain comprising the amino acid sequence of SEQ ID NO: 200. In some embodiments, the antibody or antigen binding fragment provided herein comprises a heavy chain comprising the amino acid sequence of SEQ ID NO: 201, and a light chain comprising the amino acid sequence of SEQ ID NO: 202. In some embodiments, the antibody or antigen binding fragment provided herein comprises a heavy chain comprising the amino acid sequence of SEQ ID NO: 203, and a light chain comprising the amino acid sequence of SEQ ID NO: 204. In some embodiments, the antibody or antigen binding fragment provided herein comprises a heavy chain comprising the amino acid sequence of SEQ ID NO: 205, and a light chain comprising the amino acid sequence of SEQ ID NO: 206. In some embodiments, the antibody or antigen binding fragment provided herein comprises a heavy chain comprising the amino acid sequence of SEQ ID NO: 207, and a light chain comprising the amino acid sequence of SEQ ID NO: 208. In some embodiments, the antibody or antigen binding fragment provided herein comprises a heavy chain comprising the amino acid sequence of SEQ ID NO: 209, and a light chain comprising the amino acid sequence of SEQ ID NO: 210. In some embodiments, the antibody or antigen binding fragment provided herein comprises a heavy chain comprising the amino acid sequence of SEQ ID NO: 211, and a light chain comprising the amino acid sequence of SEQ ID NO: 212. In some embodiments, the antibody or antigen binding fragment provided herein comprises a heavy chain comprising the amino acid sequence of SEQ ID NO: 213, and a light chain comprising the amino acid sequence of SEQ ID NO: 214. In some embodiments, the antibody or antigen binding fragment provided herein comprises a heavy chain comprising the amino acid sequence of SEQ ID NO: 215, and a light chain comprising the amino acid sequence of SEQ ID NO: 216. In some embodiments, the antibody or antigen binding fragment provided herein comprises a heavy chain comprising the amino acid sequence of SEQ ID NO: 217, and a light chain comprising the amino acid sequence of SEQ ID NO: 218. In some embodiments, the antibody or antigen binding fragment provided herein comprises a heavy chain comprising the amino acid sequence of SEQ ID NO: 219, and a light chain comprising the amino acid sequence of SEQ ID NO: 220. In some embodiments, the antibody or antigen binding fragment provided herein comprises a heavy chain comprising the amino acid sequence of SEQ ID NO: 221, and a light chain comprising the amino acid sequence of SEQ ID NO: 222. In some embodiments, the antibody or antigen binding fragment provided herein comprises a heavy chain comprising the amino acid sequence of SEQ ID NO: 223, and a light chain comprising the amino acid sequence of SEQ ID NO: 224. In some embodiments, the antibody or antigen binding fragment provided herein comprises a heavy chain comprising the amino acid sequence of SEQ ID NO: 225, and a light chain comprising the amino acid sequence of SEQ ID NO: 226. In some embodiments, the antibody or antigen binding fragment provided herein comprises a heavy chain comprising the amino acid sequence of SEQ ID NO: 227, and a light chain comprising the amino acid sequence of SEQ ID NO: 228. In some embodiments, the antibody or antigen binding fragment provided herein comprises a heavy chain comprising the amino acid sequence of SEQ ID NO: 229, and a light chain comprising the amino acid sequence of SEQ ID NO: 230. In some embodiments, the antibody or antigen binding fragment provided herein comprises a heavy chain comprising the amino acid sequence of SEQ ID NO: 231, and a light chain comprising the amino acid sequence of SEQ ID NO: 232. In some embodiments, the antibody or antigen binding fragment provided herein comprises a heavy chain comprising the amino acid sequence of SEQ ID NO: 233, and a light chain comprising the amino acid sequence of SEQ ID NO: 234. In some embodiments, the antibody or antigen binding fragment provided herein comprises a heavy chain comprising the amino acid sequence of SEQ ID NO: 235, and a light chain comprising the amino acid sequence of SEQ ID NO: 236. In some embodiments, the antibody or antigen binding fragment provided herein comprises a heavy chain comprising the amino acid sequence of SEQ ID NO: 237, and a light chain comprising the amino acid sequence of SEQ ID NO: 238. In some embodiments, the antibody or antigen binding fragment provided herein comprises a heavy chain comprising the amino acid sequence of SEQ ID NO: 239, and a light chain comprising the amino acid sequence of SEQ ID NO: 240. In some embodiments, the antibody or antigen binding fragment provided herein comprises a heavy chain comprising the amino acid sequence of SEQ ID NO: 241, and a light chain comprising the amino acid sequence of SEQ ID NO: 242. In some embodiments, the antibody or antigen binding fragment provided herein comprises a heavy chain comprising the amino acid sequence of SEQ ID NO: 243, and a light chain comprising the amino acid sequence of SEQ ID NO: 244. In some embodiments, the antibody or antigen binding fragment provided herein comprises a heavy chain comprising the amino acid sequence of SEQ ID NO: 245, and a light chain comprising the amino acid sequence of SEQ ID NO: 246. In some embodiments, the antibody or antigen binding fragment provided herein comprises a heavy chain comprising the amino acid sequence of SEQ ID NO: 247, and a light chain comprising the amino acid sequence of SEQ ID NO: 248. In some embodiments, the antibody or antigen binding fragment provided herein comprises a heavy chain comprising the amino acid sequence of SEQ ID NO: 249, and a light chain comprising the amino acid sequence of SEQ ID NO: 250. In some embodiments, the antibody or antigen binding fragment provided herein comprises a heavy chain comprising the amino acid sequence of SEQ ID NO: 251, and a light chain comprising the amino acid sequence of SEQ ID NO: 252. In some embodiments, the antibody or antigen binding fragment provided herein comprises a heavy chain comprising the amino acid sequence of SEQ ID NO: 253, and a light chain comprising the amino acid sequence of SEQ ID NO: 254. In some embodiments, the antibody or antigen binding fragment provided herein comprises a heavy chain comprising the amino acid sequence of SEQ ID NO: 255, and a light chain comprising the amino acid sequence of SEQ ID NO: 256. In some embodiments, the antibody or antigen binding fragment provided herein comprises a heavy chain comprising the amino acid sequence of SEQ ID NO: 257, and a light chain comprising the amino acid sequence of SEQ ID NO: 258. In some embodiments, the antibody or antigen binding fragment provided herein comprises a heavy chain comprising the amino acid sequence of SEQ ID NO: 259, and a light chain comprising the amino acid sequence of SEQ ID NO: 260. In some embodiments, the antibody or antigen binding fragment provided herein comprises a heavy chain comprising the amino acid sequence of SEQ ID NO: 261, and a light chain comprising the amino acid sequence of SEQ ID NO: 262. In some embodiments, the antibody or antigen binding fragment provided herein comprises a heavy chain comprising the amino acid sequence of SEQ ID NO: 263, and a light chain comprising the amino acid sequence of SEQ ID NO: 264. In some embodiments, the antibody or antigen binding fragment provided herein comprises a heavy chain comprising the amino acid sequence of SEQ ID NO: 265, and a light chain comprising the amino acid sequence of SEQ ID NO: 266. In some embodiments, the antibody or antigen binding fragment provided herein comprises a heavy chain comprising the amino acid sequence of SEQ ID NO: 267, and a light chain comprising the amino acid sequence of SEQ ID NO: 268. In some embodiments, the antibody or antigen binding fragment provided herein comprises a heavy chain comprising the amino acid sequence of SEQ ID NO: 269, and a light chain comprising the amino acid sequence of SEQ ID NO: 270. In some embodiments, the antibody or antigen binding fragment provided herein comprises a heavy chain comprising the amino acid sequence of SEQ ID NO: 271, and a light chain comprising the amino acid sequence of SEQ ID NO: 272. In some embodiments, the antibody or antigen binding fragment provided herein comprises a heavy chain comprising the amino acid sequence of SEQ ID NO: 273, and a light chain comprising the amino acid sequence of SEQ ID NO: 274. In some embodiments, the antibody or antigen binding fragment provided herein comprises a heavy chain comprising the amino acid sequence of SEQ ID NO: 275, and a light chain comprising the amino acid sequence of SEQ ID NO: 276. In some embodiments, the antibody or antigen binding fragment provided herein comprises a heavy chain comprising the amino acid sequence of SEQ ID NO: 277, and a light chain comprising the amino acid sequence of SEQ ID NO: 278. In some embodiments, the antibody or antigen binding fragment provided herein comprises a heavy chain comprising the amino acid sequence of SEQ ID NO: 279, and a light chain comprising the amino acid sequence of SEQ ID NO: 280. In some embodiments, the antibody or antigen binding fragment provided herein comprises a heavy chain comprising the amino acid sequence of SEQ ID NO: 281, and a light chain comprising the amino acid sequence of SEQ ID NO: 282. In some embodiments, the antibody or antigen binding fragment provided herein comprises a heavy chain comprising the amino acid sequence of SEQ ID NO: 1143, and a light chain comprising the amino acid sequence of SEQ ID NO: 1144. In some embodiments, the antibody or antigen binding fragment provided herein comprises a heavy chain comprising the amino acid sequence of SEQ ID NO: 1145, and a light chain comprising the amino acid sequence of SEQ ID NO: 1146. In some embodiments, the antibody or antigen binding fragment provided herein comprises a heavy chain comprising the amino acid sequence of SEQ ID NO: 1147, and a light chain comprising the amino acid sequence of SEQ ID NO: 1148. In some embodiments, the antibody or antigen binding fragment provided herein comprises a heavy chain comprising the amino acid sequence of SEQ ID NO: 1149, and a light chain comprising the amino acid sequence of SEQ ID NO: 1150. In some embodiments, the antibody or antigen binding fragment provided herein comprises a heavy chain comprising the amino acid sequence of SEQ ID NO: 1151, and a light chain comprising the amino acid sequence of SEQ ID NO: 1152. In some embodiments, the antibody or antigen binding fragment provided herein comprises a heavy chain comprising the amino acid sequence of SEQ ID NO: 1153, and a light chain comprising the amino acid sequence of SEQ ID NO: 1154.

In some embodiments, provided herein is a LILRB2 binding protein comprising any one of the anti-LILRB2 antibodies described above. In some embodiments, the LILRB2 binding protein is a monoclonal antibody, including a mouse, chimeric, humanized or human antibody. In some embodiments, the anti-LILRB2 antibody is an antibody fragment, e.g., a scFv. In some embodiments, the LILRB2 binding protein is a fusion protein comprising the anti-LILRB2 antibody provided herein. In other embodiments, the LILRB2 binding protein is a multispecific antibody comprising the anti-LILRB2 antibody provided herein. Other exemplary LILRB2 binding molecules are described in more detail in the following sections.

In some embodiments, the anti-LILRB2 antibody or antigen binding protein according to any of the above embodiments may incorporate any of the features, singly or in combination, as described in Sections 5.2.4 to 5.2.7 below.

In some embodiments, provided herein are antibodies that specifically bind to LILRB2 and can modulate LILRB2 activity and/or expression (e.g., inhibit LILRB2 mediated signaling). LILRB2 activity or LILRB2 signaling activity includes, but is not limited to, suppression of myeloid cells, suppression of myeloid cell activity, suppression of tumor-associated myeloid cells, suppression of NK cells, and suppression of cytolytic T-cells. In certain embodiments, a LILRB2 antagonist is provided herein that is an antibody described herein that specifically binds to LILRB2 and inhibits (including partially inhibits) at least one LILRB2 activity. In some embodiments, an anti-LILRB2 antibody inhibits LILRB2 signaling thereby reversing an LILRB2-induced suppressive effect. In some embodiments, the antibodies provided herein inhibit (including partially inhibit or reduce) the binding of LILRB2 to its ligands. In some embodiments, the ligands are MHC I molecules. In some embodiments, the ligand is HLA-A2. In some embodiments, the ligand is HLA-G . . . . In some embodiments, the ligand is ANGPTLs. A LILRB2 activity can relate to any activity of LILRB2 such as those known or described in the art. In certain embodiments, LILRB2 activity and LILRB2 signaling (or LILRB2 mediated signaling) are used interchangeably herein.

In some embodiments, the antibody provided herein inhibits the interaction between LILRB2 and ligands of LILRB2 by 10%-99%. In other embodiments, the antibody provided herein inhibits the interaction between LILRB2 and ligands of LILRB2 100% (i.e., completely abolish the interaction as measured by an assay). In some embodiments, the antibody provided herein inhibits the interaction between LILRB2 and ligands of LILRB2 by at least 10%. In some embodiments, the antibody provided herein inhibits the interaction between LILRB2 and ligands of LILRB2 by at least 20%. In some embodiments, the antibody provided herein inhibits the interaction between LILRB2 and ligands of LILRB2 by at least 30%. In some embodiments, the antibody provided herein inhibits the interaction between LILRB2 and ligands of LILRB2 by at least 40%. In some embodiments, the antibody provided herein inhibits the interaction between LILRB2 and ligands of LILRB2 by at least 50%. In some embodiments, the antibody provided herein inhibits the interaction between LILRB2 and ligands of LILRB2 by at least 60%. In some embodiments, the antibody provided herein inhibits the interaction between LILRB2 and ligands of LILRB2 by at least 70%. In some embodiments, the antibody provided herein inhibits the interaction between LILRB2 and ligands of LILRB2 by at least 80%. In some embodiments, the antibody provided herein inhibits the interaction between LILRB2 and ligands of LILRB2 by at least 90%. In some embodiments, the antibody provided herein inhibits the interaction between LILRB2 and ligands of LILRB2 by about 10%-90%. In some embodiments, the antibody provided herein inhibits the interaction between LILRB2 and ligands of LILRB2 by about 20%-80%. In some embodiments, the antibody provided herein inhibits the interaction between LILRB2 and ligands of LILRB2 by about 30%-70%. In some embodiments, the antibody provided herein inhibits the interaction between LILRB2 and ligands of LILRB2 by about 40%-60%. In some embodiments, the LILRB2 and the ligands of LILRB2 are expressed on different cells. In some embodiments, the LILRB2 cell is expressed on a first cell, such as an immune cell. In some embodiments, the immune cell is an NK cell. In some embodiments, the immune cell is a monocyte. In some embodiments, the immune cell is a macrophage. In some embodiments, the immune cell is a T cell. In some embodiments, the T cell is a cytotoxic T cell such as a CD8+ T cell. In some embodiments, the immune cell is a dendritic cell. In some embodiments, the ligands are MHC I molecules. In some embodiments, the ligand is HLA-A2. In some embodiments, the ligand is HLA-G. In some embodiments, the ligand is ANGPTLs.

In certain embodiments, the antibody described herein attenuates (e.g., partially attenuates) a LILRB2 activity. In some embodiments, the antibody provided herein attenuates a LILRB2 activity by at least about 10%. In some embodiments, the antibody provided herein attenuates a LILRB2 activity by at least about 20%. In some embodiments, the antibody provided herein attenuates a LILRB2 activity by at least about 30%. In some embodiments, the antibody provided herein attenuates a LILRB2 activity by at least about 40%. In some embodiments, the antibody provided herein attenuates a LILRB2 activity by at least about 50%. In some embodiments, the antibody provided herein attenuates a LILRB2 activity by at least about 60%. In some embodiments, the antibody provided herein attenuates a LILRB2 activity by at least about 70%. In some embodiments, the antibody provided herein attenuates a LILRB2 activity by at least about 80%. In some embodiments, the antibody provided herein attenuates a LILRB2 activity by at least about 90%. In some embodiments, the antibody provided herein attenuates a LILRB2 activity by at least about 95%. In certain embodiments, the antibody described herein can attenuate (e.g., partially attenuate) a LILRB2 activity by at least about 15% to about 65%. In certain embodiments, the antibody described herein can attenuate (e.g., partially attenuate) a LILRB2 activity by at least about 20% to about 65%. In certain embodiments, the antibody described herein can attenuate (e.g., partially attenuate) a LILRB2 activity by at least about 30% to about 65%.

In specific embodiments, the attenuation of a LILRB2 activity is assessed by methods described herein. In specific embodiments, the attenuation of a LILRB2 activity is assessed by methods known to one of skill in the art. In certain embodiments, the attenuation of a LILRB2 activity is relative to the LILRB2 activity in the presence of stimulation without any anti-LILRB2 antibody. In certain embodiments, the attenuation of a LILRB2 activity is relative to the LILRB2 activity in the presence of stimulation with an unrelated antibody (e.g., an antibody that does not specifically bind to LILRB2).

A non-limiting example of a LILRB2 activity is LILRB2 mediated signaling. Thus, in certain embodiments, the antibody described herein attenuates (e.g., partially attenuates) LILRB2 mediated signaling. In some embodiments, the antibody provided herein attenuates LILRB2 mediated signaling by at least about 10%. In some embodiments, the antibody provided herein attenuates LILRB2 mediated signaling by at least about 20%. In some embodiments, the antibody provided herein attenuates LILRB2 mediated signaling by at least about 30%. In some embodiments, the antibody provided herein attenuates LILRB2 mediated signaling by at least about 40%. In some embodiments, the antibody provided herein attenuates LILRB2 mediated signaling by at least about 50%. In some embodiments, the antibody provided herein attenuates LILRB2 mediated signaling by at least about 60%. In some embodiments, the antibody provided herein attenuates LILRB2 mediated signaling by at least about 70%. In some embodiments, the antibody provided herein attenuates LILRB2 mediated signaling by at least about 80%. In some embodiments, the antibody provided herein attenuates LILRB2 mediated signaling by at least about 90%. In some embodiments, the antibody provided herein attenuates LILRB2 mediated signaling by at least about 95%. In certain embodiments, the antibody described herein can attenuate (e.g., partially attenuate) LILRB2 mediated signaling by at least about 15% to about 65%. In certain embodiments, the antibody described herein can attenuate (e.g., partially attenuate) LILRB2 mediated signaling by at least about 20% to about 65%. In certain embodiments, the antibody described herein can attenuate (e.g., partially attenuate) LILRB2 mediated signaling by at least about 30% to about 65%.

In other embodiments, the antibody described herein can prevent suppression of an immune cell, e.g., a suppression mediated by the interaction between LILRB2 expressed on the immune cell with ligands of LILRB2 (e.g., expressed on a cancer or tumor cell). In some embodiments, the ligands are MHC I molecules. In some embodiments, the ligand is HLA-A2. In some embodiments, the ligand is HLA-G. In some embodiments, the ligand is ANGPTLs. In yet other embodiments, the antibody described herein can activate a response mediated by an immune cell, e.g., an anti-tumor response. In some embodiments, the immune cell is an NK cell. In some embodiments, the immune cell is a T cell. In some embodiments, the T cell is a cytotoxic T cell such as a CD8+ T cell. In some embodiments, the immune cell is a monocyte. In some embodiments, the immune cell is a macrophage. In some embodiments, the immune cell is a dendritic cell. In some embodiments, the immune cell mediates an anti-cancer/tumor response. In further embodiments, the cancer or tumor cell expresses HLA-A2. In some embodiments, the cancer or tumor cell expresses is HLA-G. In some embodiments, the ligand is ANGPTLs.

In some embodiments, the antibody described herein can increases myeloid cell activity. In some embodiments, the antibody described herein can enhance the activation of NK cells. In some embodiments, the antibody described herein can enhance macrophage activity. In some embodiments, the antibody described herein can enhance macrophage phagocytosis. In some embodiments, the antibody described herein can enhance T cell activity. In some embodiments, the antibody described herein can enhance the activity of CD8+ T cells. In some embodiments, the antibody described herein can enhance dendritic cell activity. In some embodiments, the antibody described herein can enhance FcR-mediated dendritic cell activity. In some embodiments, the antibody described herein can enhance cytokine release by immune cells. In some embodiments, the antibody described herein can enhance TNFα release by immune cells. In some embodiments, the antibody described herein can enhance IFN-γ release by immune cells.

In some embodiments, the antibody described herein enhances anti-tumor immunity by at least 10%. In some embodiments, the antibody described herein enhances anti-tumor immunity by at least 20%. In some embodiments, the antibody described herein enhances anti-tumor immunity by at least 30%. In some embodiments, the antibody described herein enhances anti-tumor immunity by at least 40%. In some embodiments, the antibody described herein enhances anti-tumor immunity by at least 50%. In some embodiments, the antibody described herein enhances anti-tumor immunity by at least 60%. In some embodiments, the antibody described herein enhances anti-tumor immunity by at least 70%. In some embodiments, the antibody described herein enhances anti-tumor immunity by at least 80%. In some embodiments, the antibody described herein enhances anti-tumor immunity by at least 90%. In some embodiments, the antibody described herein enhances anti-tumor immunity by at least 95%. In certain embodiments, the antibody described herein enhances anti-tumor immunity by at least about 15% to about 65%. In certain embodiments, the antibody described herein enhances anti-tumor immunity by at least about 20% to about 65%. In certain embodiments, the antibody described herein enhances anti-tumor immunity by at least about 30% to about 65%.

### 5.2.3 Multispecific Binding Agents

In one aspect, the binding agents provided herein are multispecific binding agents that bind to both LILRB1 and LILRB2.

In certain embodiments, the present multispecific binding agent is a multispecific antibody comprising a first binding domain that binds to LILRB1 and a second binding domain that binds to LILRB2.

In some embodiments of the multispecific antibody provided herein, the first binding domain that binds to LILRB1 comprises any antibody or antigen binding fragment described in Section 5.2.1 above. In some embodiments of the multispecific antibody provided herein, the second binding domain that binds to LILRB2 comprises any antibody or antigen binding fragment described in Section 5.2.2 above.

In some embodiments of the multispecific antibody provided herein, the first binding domain comprises six CDRs of any anti-LILRB1 antibody or antigen binding fragment described in Section 5.2.1 above. In some embodiments of the multispecific antibody provided herein, the second binding domain comprises six CDRs of any anti-LILRB2 antibody or antigen binding fragment described in Section 5.2.2 above.

In some embodiments of the multispecific antibody provided herein, the first binding domain comprises a VH and a VL of any anti-LILRB1 antibody or antigen binding fragment described in Section 5.2.1 above. In some embodiments of the multispecific antibody provided herein, the second binding domain comprises a VH and a VL of any anti-LILRB2 antibody or antigen binding fragments as described in Section 5.2.2 above.

In some embodiments of the multispecific antibody provided herein, the first binding domain comprises six CDRs of any anti-LILRB1 antibody or antigen binding fragment described in Tables 5-6, and 14-15. In some embodiments of the multispecific antibody provided herein, the second binding domain comprises six CDRs of any anti-LILRB2 antibody or antigen binding fragment described in Tables 5-6, and 14-15.

In some embodiments of the multispecific antibody provided herein, the first binding domain comprises a VH and a VL of any anti-LILRB1 antibody or antigen binding fragment described in Tables 5 and 14. In some embodiments of the multispecific antibody provided herein, the second binding domain comprises a VH and a VL of any anti-LILRB2 antibody or antigen binding fragments as described in Tables 5 and 14.

In some embodiments, provided herein is a multispecific binding agent comprising a first binding domain that binds to LILRB1 comprising a HCDR1, a HCDR2, and a HCDR3 as set forth in a VH comprising the amino acid sequence of SEQ ID NO: 1155 and a LCDR1, a LCDR2, and a LCDR3 as set forth in a VL comprising the amino acid sequence of SEQ ID NO: 1156; and a second binding domain that binds to LILRB2 comprising a HCDR1, a HCDR2, and a HCDR3 as set forth in a VH comprising the amino acid sequence of SEQ ID NO: 1167 and a LCDR1, a LCDR2, and a LCDR3 as set forth in a VL comprising the amino acid sequence of SEQ ID NO: 1168.

In some embodiments, provided herein is a multispecific binding agent comprising a first binding domain that binds to LILRB1 comprising a HCDR1, a HCDR2, and a HCDR3 as set forth in a VH comprising the amino acid sequence of SEQ ID NO: 1155 and a LCDR1, a LCDR2, and a LCDR3 as set forth in a VL comprising the amino acid sequence of SEQ ID NO: 1156; and a second binding domain that binds to LILRB2 comprising a HCDR1, a HCDR2, and a HCDR3 as set forth in a VH comprising the amino acid sequence of SEQ ID NO: 1169 and a LCDR1, a LCDR2, and a LCDR3 as set forth in a VL comprising the amino acid sequence of SEQ ID NO: 1170.

In some embodiments, provided herein is a multispecific binding agent comprising a first binding domain that binds to LILRB1 comprising a HCDR1, a HCDR2, and a HCDR3 as set forth in a VH comprising the amino acid sequence of SEQ ID NO: 1155 and a LCDR1, a LCDR2, and a LCDR3 as set forth in a VL comprising the amino acid sequence of SEQ ID NO: 1156; and a second binding domain that binds to LILRB2 comprising a HCDR1, a HCDR2, and a HCDR3 as set forth in a VH comprising the amino acid sequence of SEQ ID NO: 1171 and a LCDR1, a LCDR2, and a LCDR3 as set forth in a VL comprising the amino acid sequence of SEQ ID NO: 1172.

In some embodiments, provided herein is a multispecific binding agent comprising a first binding domain that binds to LILRB1 comprising a HCDR1, a HCDR2, and a HCDR3 as set forth in a VH comprising the amino acid sequence of SEQ ID NO: 1155 and a LCDR1, a LCDR2, and a LCDR3 as set forth in a VL comprising the amino acid sequence of SEQ ID NO: 1156; and a second binding domain that binds to LILRB2 comprising a HCDR1, a HCDR2, and a HCDR3 as set forth in a VH comprising the amino acid sequence of SEQ ID NO: 1173 and a LCDR1, a LCDR2, and a LCDR3 as set forth in a VL comprising the amino acid sequence of SEQ ID NO: 1174.

In some embodiments, provided herein is a multispecific binding agent a first binding domain that binds to LILRB1 comprising a HCDR1, a HCDR2, and a HCDR3 as set forth in a VH comprising the amino acid sequence of SEQ ID NO: 1155 and a LCDR1, a LCDR2, and a LCDR3 as set forth in a VL comprising the amino acid sequence of SEQ ID NO: 1156; and a second binding domain that binds to LILRB2 comprising a HCDR1, a HCDR2, and a HCDR3 as set forth in a VH comprising the amino acid sequence of SEQ ID NO: 1175 and a LCDR1, a LCDR2, and a LCDR3 as set forth in a VL comprising the amino acid sequence of SEQ ID NO: 1176.

In some embodiments, provided herein is a multispecific binding agent comprising a first binding domain that binds to LILRB1 comprising a HCDR1, a HCDR2, and a HCDR3 as set forth in a VH comprising the amino acid sequence of SEQ ID NO: 1155 and a LCDR1, a LCDR2, and a LCDR3 as set forth in a VL comprising the amino acid sequence of SEQ ID NO: 1156; and a second binding domain that binds to LILRB2 comprising a HCDR1, a HCDR2, and a HCDR3 as set forth in a VH comprising the amino acid sequence of SEQ ID NO: 1177 and a LCDR1, a LCDR2, and a LCDR3 as set forth in a VL comprising the amino acid sequence of SEQ ID NO: 1178.

In some embodiments, provided herein is a multispecific binding agent comprising a first binding domain that binds to LILRB1 comprising a HCDR1, a HCDR2, and a HCDR3 as set forth in a VH comprising the amino acid sequence of SEQ ID NO: 1157 and a LCDR1, a LCDR2, and a LCDR3 as set forth in a VL comprising the amino acid sequence of SEQ ID NO: 1158; and a second binding domain that binds to LILRB2 comprising a HCDR1, a HCDR2, and a HCDR3 as set forth in a VH comprising the amino acid sequence of SEQ ID NO: 1167 and a LCDR1, a LCDR2, and a LCDR3 as set forth in a VL comprising the amino acid sequence of SEQ ID NO: 1168.

In some embodiments, provided herein is a multispecific binding agent comprising a first binding domain that binds to LILRB1 comprising a HCDR1, a HCDR2, and a HCDR3 as set forth in a VH comprising the amino acid sequence of SEQ ID NO: 1157 and a LCDR1, a LCDR2, and a LCDR3 as set forth in a VL comprising the amino acid sequence of SEQ ID NO: 1158; and a second binding domain that binds to LILRB2 comprising a HCDR1, a HCDR2, and a HCDR3 as set forth in a VH comprising the amino acid sequence of SEQ ID NO: 1169 and a LCDR1, a LCDR2, and a LCDR3 as set forth in a VL comprising the amino acid sequence of SEQ ID NO: 1170.

In some embodiments, provided herein is a multispecific binding agent comprising a first binding domain that binds to LILRB1 comprising a HCDR1, a HCDR2, and a HCDR3 as set forth in a VH comprising the amino acid sequence of SEQ ID NO: 1157 and a LCDR1, a LCDR2, and a LCDR3 as set forth in a VL comprising the amino acid sequence of SEQ ID NO: 1158; and a second binding domain that binds to LILRB2 comprising a HCDR1, a HCDR2, and a HCDR3 as set forth in a VH comprising the amino acid sequence of SEQ ID NO: 1171 and a LCDR1, a LCDR2, and a LCDR3 as set forth in a VL comprising the amino acid sequence of SEQ ID NO: 1172.

In some embodiments, provided herein is a multispecific binding agent comprising a first binding domain that binds to LILRB1 comprising a HCDR1, a HCDR2, and a HCDR3 as set forth in a VH comprising the amino acid sequence of SEQ ID NO: 1157 and a LCDR1, a LCDR2, and a LCDR3 as set forth in a VL comprising the amino acid sequence of SEQ ID NO: 1158; and a second binding domain that binds to LILRB2 comprising a HCDR1, a HCDR2, and a HCDR3 as set forth in a VH comprising the amino acid sequence of SEQ ID NO: 1173 and a LCDR1, a LCDR2, and a LCDR3 as set forth in a VL comprising the amino acid sequence of SEQ ID NO: 1174.

In some embodiments, provided herein is a multispecific binding agent comprising a first binding domain that binds to LILRB1 comprising a HCDR1, a HCDR2, and a HCDR3 as set forth in a VH comprising the amino acid sequence of SEQ ID NO: 1157 and a LCDR1, a LCDR2, and a LCDR3 as set forth in a VL comprising the amino acid sequence of SEQ ID NO: 1158; and a second binding domain that binds to LILRB2 comprising a HCDR1, a HCDR2, and a HCDR3 as set forth in a VH comprising the amino acid sequence of SEQ ID NO: 1175 and a LCDR1, a LCDR2, and a LCDR3 as set forth in a VL comprising the amino acid sequence of SEQ ID NO: 1176.

In some embodiments, provided herein is a multispecific binding agent comprising a first binding domain that binds to LILRB1 comprising a HCDR1, a HCDR2, and a HCDR3 as set forth in a VH comprising the amino acid sequence of SEQ ID NO: 1157 and a LCDR1, a LCDR2, and a LCDR3 as set forth in a VL comprising the amino acid sequence of SEQ ID NO: 1158; and a second binding domain that binds to LILRB2 comprising a HCDR1, a HCDR2, and a HCDR3 as set forth in a VH comprising the amino acid sequence of SEQ ID NO: 1177 and a LCDR1, a LCDR2, and a LCDR3 as set forth in a VL comprising the amino acid sequence of SEQ ID NO: 1178.

In some embodiments, provided herein is a multispecific binding agent comprising a first binding domain that binds to LILRB1 comprising a HCDR1, a HCDR2, and a HCDR3 as set forth in a VH comprising the amino acid sequence of SEQ ID NO: 1159 and a LCDR1, a LCDR2, and a LCDR3 as set forth in a VL comprising the amino acid sequence of SEQ ID NO: 1160; and a second binding domain that binds to LILRB2 comprising a HCDR1, a HCDR2, and a HCDR3 as set forth in a VH comprising the amino acid sequence of SEQ ID NO: 1167 and a LCDR1, a LCDR2, and a LCDR3 as set forth in a VL comprising the amino acid sequence of SEQ ID NO: 1168.

In some embodiments, provided herein is a multispecific binding agent comprising a first binding domain that binds to LILRB1 comprising a HCDR1, a HCDR2, and a HCDR3 as set forth in a VH comprising the amino acid sequence of SEQ ID NO: 1159 and a LCDR1, a LCDR2, and a LCDR3 as set forth in a VL comprising the amino acid sequence of SEQ ID NO: 1160; and a second binding domain that binds to LILRB2 comprising a HCDR1, a HCDR2, and a HCDR3 as set forth in a VH comprising the amino acid sequence of SEQ ID NO: 1169 and a LCDR1, a LCDR2, and a LCDR3 as set forth in a VL comprising the amino acid sequence of SEQ ID NO: 1170.

In some embodiments, provided herein is a multispecific binding agent comprising a first binding domain that binds to LILRB1 comprising a HCDR1, a HCDR2, and a HCDR3 as set forth in a VH comprising the amino acid sequence of SEQ ID NO: 1159 and a LCDR1, a LCDR2, and a LCDR3 as set forth in a VL comprising the amino acid sequence of SEQ ID NO: 1160; and a second binding domain that binds to LILRB2 comprising a HCDR1, a HCDR2, and a HCDR3 as set forth in a VH comprising the amino acid sequence of SEQ ID NO: 1171 and a LCDR1, a LCDR2, and a LCDR3 as set forth in a VL comprising the amino acid sequence of SEQ ID NO: 1172.

In some embodiments, provided herein is a multispecific binding agent comprising a first binding domain that binds to LILRB1 comprising a HCDR1, a HCDR2, and a HCDR3 as set forth in a VH comprising the amino acid sequence of SEQ ID NO: 1159 and a LCDR1, a LCDR2, and a LCDR3 as set forth in a VL comprising the amino acid sequence of SEQ ID NO: 1160; and a second binding domain that binds to LILRB2 comprising a HCDR1, a HCDR2, and a HCDR3 as set forth in a VH comprising the amino acid sequence of SEQ ID NO: 1173 and a LCDR1, a LCDR2, and a LCDR3 as set forth in a VL comprising the amino acid sequence of SEQ ID NO: 1174.

In some embodiments, provided herein is a multispecific binding agent comprising a first binding domain that binds to LILRB1 comprising a HCDR1, a HCDR2, and a HCDR3 as set forth in a VH comprising the amino acid sequence of SEQ ID NO: 1159 and a LCDR1, a LCDR2, and a LCDR3 as set forth in a VL comprising the amino acid sequence of SEQ ID NO: 1160; and a second binding domain that binds to LILRB2 comprising a HCDR1, a HCDR2, and a HCDR3 as set forth in a VH comprising the amino acid sequence of SEQ ID NO: 1175 and a LCDR1, a LCDR2, and a LCDR3 as set forth in a VL comprising the amino acid sequence of SEQ ID NO: 1176.

In some embodiments, provided herein is a multispecific binding agent comprising a first binding domain that binds to LILRB1 comprising a HCDR1, a HCDR2, and a HCDR3 as set forth in a VH comprising the amino acid sequence of SEQ ID NO: 1159 and a LCDR1, a LCDR2, and a LCDR3 as set forth in a VL comprising the amino acid sequence of SEQ ID NO: 1160; and a second binding domain that binds to LILRB2 comprising a HCDR1, a HCDR2, and a HCDR3 as set forth in a VH comprising the amino acid sequence of SEQ ID NO: 1177 and a LCDR1, a LCDR2, and a LCDR3 as set forth in a VL comprising the amino acid sequence of SEQ ID NO: 1178.

In some embodiments, provided herein is a multispecific binding agent comprising a first binding domain that binds to LILRB1 comprising a HCDR1, a HCDR2, and a HCDR3 as set forth in a VH comprising the amino acid sequence of SEQ ID NO: 1161 and a LCDR1, a LCDR2, and a LCDR3 as set forth in a VL comprising the amino acid sequence of SEQ ID NO: 1162; and a second binding domain that binds to LILRB2 comprising a HCDR1, a HCDR2, and a HCDR3 as set forth in a VH comprising the amino acid sequence of SEQ ID NO: 1167 and a LCDR1, a LCDR2, and a LCDR3 as set forth in a VL comprising the amino acid sequence of SEQ ID NO: 1168.

In some embodiments, provided herein is a multispecific binding agent comprising a first binding domain that binds to LILRB1 comprising a HCDR1, a HCDR2, and a HCDR3 as set forth in a VH comprising the amino acid sequence of SEQ ID NO: 1161 and a LCDR1, a LCDR2, and a LCDR3 as set forth in a VL comprising the amino acid sequence of SEQ ID NO: 1162; and a second binding domain that binds to LILRB2 comprising a HCDR1, a HCDR2, and a HCDR3 as set forth in a VH comprising the amino acid sequence of SEQ ID NO: 1169 and a LCDR1, a LCDR2, and a LCDR3 as set forth in a VL comprising the amino acid sequence of SEQ ID NO: 1170

In some embodiments, provided herein is a multispecific binding agent comprising a first binding domain that binds to LILRB1 comprising a HCDR1, a HCDR2, and a HCDR3 as set forth in a VH comprising the amino acid sequence of SEQ ID NO: 1161 and a LCDR1, a LCDR2, and a LCDR3 as set forth in a VL comprising the amino acid sequence of SEQ ID NO: 1162; and a second binding domain that binds to LILRB2 comprising a HCDR1, a HCDR2, and a HCDR3 as set forth in a VH comprising the amino acid sequence of SEQ ID NO: 1171 and a LCDR1, a LCDR2, and a LCDR3 as set forth in a VL comprising the amino acid sequence of SEQ ID NO: 1172.

In some embodiments, provided herein is a multispecific binding agent comprising a first binding domain that binds to LILRB1 comprising a HCDR1, a HCDR2, and a HCDR3 as set forth in a VH comprising the amino acid sequence of SEQ ID NO: 1161 and a LCDR1, a LCDR2, and a LCDR3 as set forth in a VL comprising the amino acid sequence of SEQ ID NO: 1162; and a second binding domain that binds to LILRB2 comprising a HCDR1, a HCDR2, and a HCDR3 as set forth in a VH comprising the amino acid sequence of SEQ ID NO: 1173 and a LCDR1, a LCDR2, and a LCDR3 as set forth in a VL comprising the amino acid sequence of SEQ ID NO: 1174.

In some embodiments, provided herein is a multispecific binding agent comprising a first binding domain that binds to LILRB1 comprising a HCDR1, a HCDR2, and a HCDR3 as set forth in a VH comprising the amino acid sequence of SEQ ID NO: 1161 and a LCDR1, a LCDR2, and a LCDR3 as set forth in a VL comprising the amino acid sequence of SEQ ID NO: 1162; and a second binding domain that binds to LILRB2 comprising a HCDR1, a HCDR2, and a HCDR3 as set forth in a VH comprising the amino acid sequence of SEQ ID NO: 1175 and a LCDR1, a LCDR2, and a LCDR3 as set forth in a VL comprising the amino acid sequence of SEQ ID NO: 1176.

In some embodiments, provided herein is a multispecific binding agent comprising a first binding domain that binds to LILRB1 comprising a HCDR1, a HCDR2, and a HCDR3 as set forth in a VH comprising the amino acid sequence of SEQ ID NO: 1161 and a LCDR1, a LCDR2, and a LCDR3 as set forth in a VL comprising the amino acid sequence of SEQ ID NO: 1162; and a second binding domain that binds to LILRB2 comprising a HCDR1, a HCDR2, and a HCDR3 as set forth in a VH comprising the amino acid sequence of SEQ ID NO: 1177 and a LCDR1, a LCDR2, and a LCDR3 as set forth in a VL comprising the amino acid sequence of SEQ ID NO: 1178.

In some embodiments, provided herein is a multispecific binding agent comprising a first binding domain that binds to LILRB1 comprising a HCDR1, a HCDR2, and a HCDR3 as set forth in a VH comprising the amino acid sequence of SEQ ID NO: 1163 and a LCDR1, a LCDR2, and a LCDR3 as set forth in a VL comprising the amino acid sequence of SEQ ID NO: 1164; and a second binding domain that binds to LILRB2 comprising a HCDR1, a HCDR2, and a HCDR3 as set forth in a VH comprising the amino acid sequence of SEQ ID NO: 1167 and a LCDR1, a LCDR2, and a LCDR3 as set forth in a VL comprising the amino acid sequence of SEQ ID NO: 1168.

In some embodiments, provided herein is a multispecific binding agent comprising a first binding domain that binds to LILRB1 comprising a HCDR1, a HCDR2, and a HCDR3 as set forth in a VH comprising the amino acid sequence of SEQ ID NO: 1163 and a LCDR1, a LCDR2, and a LCDR3 as set forth in a VL comprising the amino acid sequence of SEQ ID NO: 1164; and a second binding domain that binds to LILRB2 comprising a HCDR1, a HCDR2, and a HCDR3 as set forth in a VH comprising the amino acid sequence of SEQ ID NO: 1169 and a LCDR1, a LCDR2, and a LCDR3 as set forth in a VL comprising the amino acid sequence of SEQ ID NO: 1170.

In some embodiments, provided herein is a multispecific binding agent comprising a first binding domain that binds to LILRB1 comprising a HCDR1, a HCDR2, and a HCDR3 as set forth in a VH comprising the amino acid sequence of SEQ ID NO: 1163 and a LCDR1, a LCDR2, and a LCDR3 as set forth in a VL comprising the amino acid sequence of SEQ ID NO: 1164; and a second binding domain that binds to LILRB2 comprising a HCDR1, a HCDR2, and a HCDR3 as set forth in a VH comprising the amino acid sequence of SEQ ID NO: 1171 and a LCDR1, a LCDR2, and a LCDR3 as set forth in a VL comprising the amino acid sequence of SEQ ID NO: 1172

In some embodiments, provided herein is a multispecific binding agent comprising a first binding domain that binds to LILRB1 comprising a HCDR1, a HCDR2, and a HCDR3 as set forth in a VH comprising the amino acid sequence of SEQ ID NO: 1163 and a LCDR1, a LCDR2, and a LCDR3 as set forth in a VL comprising the amino acid sequence of SEQ ID NO: 1164; and a second binding domain that binds to LILRB2 comprising a HCDR1, a HCDR2, and a HCDR3 as set forth in a VH comprising the amino acid sequence of SEQ ID NO: 1173 and a LCDR1, a LCDR2, and a LCDR3 as set forth in a VL comprising the amino acid sequence of SEQ ID NO: 1174.

In some embodiments, provided herein is a multispecific binding agent comprising a first binding domain that binds to LILRB1 comprising a HCDR1, a HCDR2, and a HCDR3 as set forth in a VH comprising the amino acid sequence of SEQ ID NO: 1163 and a LCDR1, a LCDR2, and a LCDR3 as set forth in a VL comprising the amino acid sequence of SEQ ID NO: 1164; and a second binding domain that binds to LILRB2 comprising a HCDR1, a HCDR2, and a HCDR3 as set forth in a VH comprising the amino acid sequence of SEQ ID NO: 1175 and a LCDR1, a LCDR2, and a LCDR3 as set forth in a VL comprising the amino acid sequence of SEQ ID NO: 1176

In some embodiments, provided herein is a multispecific binding agent comprising a first binding domain that binds to LILRB1 comprising a HCDR1, a HCDR2, and a HCDR3 as set forth in a VH comprising the amino acid sequence of SEQ ID NO: 1163 and a LCDR1, a LCDR2, and a LCDR3 as set forth in a VL comprising the amino acid sequence of SEQ ID NO: 1164; and a second binding domain that binds to LILRB2 comprising a HCDR1, a HCDR2, and a HCDR3 as set forth in a VH comprising the amino acid sequence of SEQ ID NO: 1177 and a LCDR1, a LCDR2, and a LCDR3 as set forth in a VL comprising the amino acid sequence of SEQ ID NO: 1178

In some embodiments, provided herein is a multispecific binding agent comprising a first binding domain that binds to LILRB1 comprising a HCDR1, a HCDR2, and a HCDR3 as set forth in a VH comprising the amino acid sequence of SEQ ID NO: 1165 and a LCDR1, a LCDR2, and a LCDR3 as set forth in a VL comprising the amino acid sequence of SEQ ID NO: 1166; and a second binding domain that binds to LILRB2 comprising a HCDR1, a HCDR2, and a HCDR3 as set forth in a VH comprising the amino acid sequence of SEQ ID NO: 1167 and a LCDR1, a LCDR2, and a LCDR3 as set forth in a VL comprising the amino acid sequence of SEQ ID NO: 1168.

In some embodiments, provided herein is a multispecific binding agent comprising a first binding domain that binds to LILRB1 comprising a HCDR1, a HCDR2, and a HCDR3 as set forth in a VH comprising the amino acid sequence of SEQ ID NO: 1165 and a LCDR1, a LCDR2, and a LCDR3 as set forth in a VL comprising the amino acid sequence of SEQ ID NO: 1166; and a second binding domain that binds to LILRB2 comprising a HCDR1, a HCDR2, and a HCDR3 as set forth in a VH comprising the amino acid sequence of SEQ ID NO: 1169 and a LCDR1, a LCDR2, and a LCDR3 as set forth in a VL comprising the amino acid sequence of SEQ ID NO: 1170.

In some embodiments, provided herein is a multispecific binding agent comprising a first binding domain that binds to LILRB1 comprising a HCDR1, a HCDR2, and a HCDR3 as set forth in a VH comprising the amino acid sequence of SEQ ID NO: 1165 and a LCDR1, a LCDR2, and a LCDR3 as set forth in a VL comprising the amino acid sequence of SEQ ID NO: 1166; and a second binding domain that binds to LILRB2 comprising a HCDR1, a HCDR2, and a HCDR3 as set forth in a VH comprising the amino acid sequence of SEQ ID NO: 1171 and a LCDR1, a LCDR2, and a LCDR3 as set forth in a VL comprising the amino acid sequence of SEQ ID NO: 1172.

In some embodiments, provided herein is a multispecific binding agent comprising a first binding domain that binds to LILRB1 comprising a HCDR1, a HCDR2, and a HCDR3 as set forth in a VH comprising the amino acid sequence of SEQ ID NO: 1165 and a LCDR1, a LCDR2, and a LCDR3 as set forth in a VL comprising the amino acid sequence of SEQ ID NO: 1166; and a second binding domain that binds to LILRB2 comprising a HCDR1, a HCDR2, and a HCDR3 as set forth in a VH comprising the amino acid sequence of SEQ ID NO: 1173 and a LCDR1, a LCDR2, and a LCDR3 as set forth in a VL comprising the amino acid sequence of SEQ ID NO: 1174.

In some embodiments, provided herein is a multispecific binding agent comprising a first binding domain that binds to LILRB1 comprising a HCDR1, a HCDR2, and a HCDR3 as set forth in a VH comprising the amino acid sequence of SEQ ID NO: 1165 and a LCDR1, a LCDR2, and a LCDR3 as set forth in a VL comprising the amino acid sequence of SEQ ID NO: 1166; and a second binding domain that binds to LILRB2 comprising a HCDR1, a HCDR2, and a HCDR3 as set forth in a VH comprising the amino acid sequence of SEQ ID NO: 1175 and a LCDR1, a LCDR2, and a LCDR3 as set forth in a VL comprising the amino acid sequence of SEQ ID NO: 1176.

In some embodiments, provided herein is a multispecific binding agent comprising a first binding domain that binds to LILRB1 comprising a HCDR1, a HCDR2, and a HCDR3 as set forth in a VH comprising the amino acid sequence of SEQ ID NO: 1165 and a LCDR1, a LCDR2, and a LCDR3 as set forth in a VL comprising the amino acid sequence of SEQ ID NO: 1166; and a second binding domain that binds to LILRB2 comprising a HCDR1, a HCDR2, and a HCDR3 as set forth in a VH comprising the amino acid sequence of SEQ ID NO: 1177 and a LCDR1, a LCDR2, and a LCDR3 as set forth in a VL comprising the amino acid sequence of SEQ ID NO: 1178.

In some embodiments, provided herein is a multispecific binding agent comprising a first binding domain that binds to LILRB1 comprising a HCDR1 comprising the amino acid sequence of SEQ ID NO: 543, a HCDR2 comprising the amino acid sequence of SEQ ID NO: 544, and a HCDR3 comprising the amino acid sequence of SEQ ID NO: 1179 and a LCDR1 comprising the amino acid sequence of SEQ ID NO: 546, a LCDR2 comprising the amino acid sequence of SEQ ID NO: 1281, and a LCDR3 comprising the amino acid sequence of SEQ ID NO: 548, and a second binding domain that binds to LILRB2 comprising a HCDR1 comprising the amino acid sequence of SEQ ID NO: 855, a HCDR2 comprising the amino acid sequence of SEQ ID NO: 856, and a HCDR3 comprising the amino acid sequence of SEQ ID NO: 857 and a LCDR1 comprising the amino acid sequence of SEQ ID NO: 858, a LCDR2 comprising the amino acid sequence of SEQ ID NO: 1282, and a LCDR3 comprising the amino acid sequence of SEQ ID NO: 860.

In some embodiments, provided herein is a multispecific binding agent comprising a first binding domain that binds to LILRB1 comprising a HCDR1 comprising the amino acid sequence of SEQ ID NO: 543, a HCDR2 comprising the amino acid sequence of SEQ ID NO: 544, and a HCDR3 comprising the amino acid sequence of SEQ ID NO: 1179 and a LCDR1 comprising the amino acid sequence of SEQ ID NO: 546, a LCDR2 comprising the amino acid sequence of SEQ ID NO: 1281, and a LCDR3 comprising the amino acid sequence of SEQ ID NO: 548, and a second binding domain that binds to LILRB2 comprising a HCDR1 comprising the amino acid sequence of SEQ ID NO: 987, a HCDR2 comprising the amino acid sequence of SEQ ID NO: 988, and a HCDR3 comprising the amino acid sequence of SEQ ID NO: 989 and a LCDR1 comprising the amino acid sequence of SEQ ID NO: 990, a LCDR2 comprising the amino acid sequence of SEQ ID NO: 991, and a LCDR3 comprising the amino acid sequence of SEQ ID NO: 992.

In some embodiments, provided herein is a multispecific binding agent comprising a first binding domain that binds to LILRB1 comprising a HCDR1 comprising the amino acid sequence of SEQ ID NO: 585, a HCDR2 comprising the amino acid sequence of SEQ ID NO: 586, and a HCDR3 comprising the amino acid sequence of SEQ ID NO: 587 and a LCDR1 comprising the amino acid sequence of SEQ ID NO: 588, a LCDR2 comprising the amino acid sequence of SEQ ID NO: 589, and a LCDR3 comprising the amino acid sequence of SEQ ID NO: 590, and a second binding domain that binds to LILRB2 comprising a HCDR1 comprising the amino acid sequence of SEQ ID NO: 855, a HCDR2 comprising the amino acid sequence of SEQ ID NO: 856, and a HCDR3 comprising the amino acid sequence of SEQ ID NO: 857 and a LCDR1 comprising the amino acid sequence of SEQ ID NO: 858, a LCDR2 comprising the amino acid sequence of SEQ ID NO: 1282, and a LCDR3 comprising the amino acid sequence of SEQ ID NO: 860.

In some embodiments, provided herein is a multispecific binding agent comprising a first binding domain that binds to LILRB1 comprising a HCDR1 comprising the amino acid sequence of SEQ ID NO: 585, a HCDR2 comprising the amino acid sequence of SEQ ID NO: 586, and a HCDR3 comprising the amino acid sequence of SEQ ID NO: 587 and a LCDR1 comprising the amino acid sequence of SEQ ID NO: 588, a LCDR2 comprising the amino acid sequence of SEQ ID NO: 589, and a LCDR3 comprising the amino acid sequence of SEQ ID NO: 590, and a second binding domain that binds to LILRB2 comprising a HCDR1 comprising the amino acid sequence of SEQ ID NO: 987, a HCDR2 comprising the amino acid sequence of SEQ ID NO: 988, and a HCDR3 comprising the amino acid sequence of SEQ ID NO: 989 and a LCDR1 comprising the amino acid sequence of SEQ ID NO: 990, a LCDR2 comprising the amino acid sequence of SEQ ID NO: 991, and a LCDR3 comprising the amino acid sequence of SEQ ID NO: 992.

In some embodiments, provided herein is a multispecific binding agent comprising a first binding domain that binds to LILRB1 comprising a VH comprising the amino acid sequence of SEQ ID NO: 1155 and a VL comprising the amino acid sequence of SEQ ID NO: 1156; and a second binding domain that binds to LILRB2 comprising a VH comprising the amino acid sequence of SEQ ID NO: 1167 and a VL comprising the amino acid sequence of SEQ ID NO: 1168. In some embodiments, provided herein is a multispecific binding agent comprising a first binding domain that binds to LILRB1 comprising a VH having at least 90% sequence identity to SEQ ID NO: 1155 and a VL having at least 90% sequence identity to SEQ ID NO: 1156; and a second binding domain that binds to LILRB2 comprising a VH having at least 90% sequence identity to SEQ ID NO: 1167 and a VL having at least 90% sequence identity to SEQ ID NO: 1168.

In some embodiments, provided herein is a multispecific binding agent comprising a first binding domain that binds to LILRB1 comprising a VH comprising the amino acid sequence of SEQ ID NO: 1155 and a VL comprising the amino acid sequence of SEQ ID NO: 1156; and a second binding domain that binds to LILRB2 comprising a VH comprising the amino acid sequence of SEQ ID NO: 1169 and a VL comprising the amino acid sequence of SEQ ID NO: 1170. In some embodiments, provided herein is a multispecific binding agent comprising a first binding domain that binds to LILRB1 comprising a VH having at least 90% sequence identity to SEQ ID NO: 1155 and a VL having at least 90% sequence identity to SEQ ID NO: 1156; and a second binding domain that binds to LILRB2 comprising a VH having at least 90% sequence identity to SEQ ID NO: 1169 and a VL having at least 90% sequence identity to SEQ ID NO: 1170.

In some embodiments, provided herein is a multispecific binding agent comprising a first binding domain that binds to LILRB1 comprising a VH comprising the amino acid sequence of SEQ ID NO: 1155 and a VL comprising the amino acid sequence of SEQ ID NO: 1156; and a second binding domain that binds to LILRB2 comprising a VH comprising the amino acid sequence of SEQ ID NO: 1171 and a VL comprising the amino acid sequence of SEQ ID NO: 1172. In some embodiments, provided herein is a multispecific binding agent comprising a first binding domain that binds to LILRB1 comprising a VH having at least 90% sequence identity to SEQ ID NO: 1155 and a VL having at least 90% sequence identity to SEQ ID NO: 1156; and a second binding domain that binds to LILRB2 comprising a VH having at least 90% sequence identity to SEQ ID NO: 1171 and a VL having at least 90% sequence identity to SEQ ID NO: 1172.

In some embodiments, provided herein is a multispecific binding agent comprising a first binding domain that binds to LILRB1 comprising a VH comprising the amino acid sequence of SEQ ID NO: 1155 and a VL comprising the amino acid sequence of SEQ ID NO: 1156; and a second binding domain that binds to LILRB2 comprising a VH comprising the amino acid sequence of SEQ ID NO: 1173 and a VL comprising the amino acid sequence of SEQ ID NO: 1174. In some embodiments, provided herein is a multispecific binding agent comprising a first binding domain that binds to LILRB1 comprising a VH having at least 90% sequence identity to SEQ ID NO: 1155 and a VL having at least 90% sequence identity to SEQ ID NO: 1156; and a second binding domain that binds to LILRB2 comprising a VH having at least 90% sequence identity to SEQ ID NO: 1173 and a VL having at least 90% sequence identity to SEQ ID NO: 1174

In some embodiments, provided herein is a multispecific binding agent a first binding domain that binds to LILRB1 comprising a VH comprising the amino acid sequence of SEQ ID NO: 1155 and a VL comprising the amino acid sequence of SEQ ID NO: 1156; and a second binding domain that binds to LILRB2 comprising a VH comprising the amino acid sequence of SEQ ID NO: 1175 and a VL comprising the amino acid sequence of SEQ ID NO: 1176. In some embodiments, provided herein is a multispecific binding agent a first binding domain that binds to LILRB1 comprising a VH having at least 90% sequence identity to SEQ ID NO: 1155 and a VL having at least 90% sequence identity to SEQ ID NO: 1156; and a second binding domain that binds to LILRB2 comprising a VH having at least 90% sequence identity to SEQ ID NO: 1175 and a VL having at least 90% sequence identity to SEQ ID NO: 1176.

In some embodiments, provided herein is a multispecific binding agent comprising a first binding domain that binds to LILRB1 comprising a VH comprising the amino acid sequence of SEQ ID NO: 1155 and a VL comprising the amino acid sequence of SEQ ID NO: 1156; and a second binding domain that binds to LILRB2 comprising a VH comprising the amino acid sequence of SEQ ID NO: 1177 and a VL comprising the amino acid sequence of SEQ ID NO: 1178. In some embodiments, provided herein is a multispecific binding agent comprising a first binding domain that binds to LILRB1 comprising a VH having at least 90% sequence identity to SEQ ID NO: 1155 and a VL having at least 90% sequence identity to SEQ ID NO: 1156; and a second binding domain that binds to LILRB2 comprising a VH having at least 90% sequence identity to SEQ ID NO: 1177 and a VL having at least 90% sequence identity to SEQ ID NO: 1178.

In some embodiments, provided herein is a multispecific binding agent comprising a first binding domain that binds to LILRB1 comprising a VH comprising the amino acid sequence of SEQ ID NO: 1157 and a VL comprising the amino acid sequence of SEQ ID NO: 1158; and a second binding domain that binds to LILRB2 comprising a VH comprising the amino acid sequence of SEQ ID NO: 1167 and a VL comprising the amino acid sequence of SEQ ID NO: 1168. In some embodiments, provided herein is a multispecific binding agent comprising a first binding domain that binds to LILRB1 comprising a VH having at least 90% sequence identity to SEQ ID NO: 1157 and a VL having at least 90% sequence identity to SEQ ID NO: 1158; and a second binding domain that binds to LILRB2 comprising a VH having at least 90% sequence identity to SEQ ID NO: 1167 and a VL having at least 90% sequence identity to SEQ ID NO: 1168.

In some embodiments, provided herein is a multispecific binding agent comprising a first binding domain that binds to LILRB1 comprising a VH comprising the amino acid sequence of SEQ ID NO: 1157 and a VL comprising the amino acid sequence of SEQ ID NO: 1158; and a second binding domain that binds to LILRB2 comprising a VH comprising the amino acid sequence of SEQ ID NO: 1169 and a VL comprising the amino acid sequence of SEQ ID NO: 1170. In some embodiments, provided herein is a multispecific binding agent comprising a first binding domain that binds to LILRB1 comprising a VH having at least 90% sequence identity to SEQ ID NO: 1157 and a VL having at least 90% sequence identity to SEQ ID NO: 1158; and a second binding domain that binds to LILRB2 comprising a VH having at least 90% sequence identity to SEQ ID NO: 1169 and a VL having at least 90% sequence identity to SEQ ID NO: 1170.

In some embodiments, provided herein is a multispecific binding agent comprising a first binding domain that binds to LILRB1 comprising a VH comprising the amino acid sequence of SEQ ID NO: 1157 and a VL comprising the amino acid sequence of SEQ ID NO: 1158; and a second binding domain that binds to LILRB2 comprising a VH comprising the amino acid sequence of SEQ ID NO: 1171 and a VL comprising the amino acid sequence of SEQ ID NO: 1172. In some embodiments, provided herein is a multispecific binding agent comprising a first binding domain that binds to LILRB1 comprising a VH having at least 90% sequence identity to SEQ ID NO: 1157 and a VL having at least 90% sequence identity to SEQ ID NO: 1158; and a second binding domain that binds to LILRB2 comprising a VH having at least 90% sequence identity to SEQ ID NO: 1171 and a VL having at least 90% sequence identity to SEQ ID NO: 1172.

In some embodiments, provided herein is a multispecific binding agent comprising a first binding domain that binds to LILRB1 comprising a VH comprising the amino acid sequence of SEQ ID NO: 1157 and a VL comprising the amino acid sequence of SEQ ID NO: 1158; and a second binding domain that binds to LILRB2 comprising a VH comprising the amino acid sequence of SEQ ID NO: 1173 and a VL comprising the amino acid sequence of SEQ ID NO: 1174. In some embodiments, provided herein is a multispecific binding agent comprising a first binding domain that binds to LILRB1 comprising a VH having at least 90% sequence identity to SEQ ID NO: 1157 and a VL having at least 90% sequence identity to SEQ ID NO: 1158; and a second binding domain that binds to LILRB2 comprising a VH having at least 90% sequence identity to SEQ ID NO: 1173 and a VL having at least 90% sequence identity to SEQ ID NO: 1174.

In some embodiments, provided herein is a multispecific binding agent comprising a first binding domain that binds to LILRB1 comprising a VH comprising the amino acid sequence of SEQ ID NO: 1157 and a VL comprising the amino acid sequence of SEQ ID NO: 1158; and a second binding domain that binds to LILRB2 comprising a VH comprising the amino acid sequence of SEQ ID NO: 1175 and a VL comprising the amino acid sequence of SEQ ID NO: 1176. In some embodiments, provided herein is a multispecific binding agent comprising a first binding domain that binds to LILRB1 comprising a VH having at least 90% sequence identity to SEQ ID NO: 1157 and a VL having at least 90% sequence identity to SEQ ID NO: 1158; and a second binding domain that binds to LILRB2 comprising a VH having at least 90% sequence identity to SEQ ID NO: 1175 and a VL having at least 90% sequence identity to SEQ ID NO: 1176.

In some embodiments, provided herein is a multispecific binding agent comprising a first binding domain that binds to LILRB1 comprising a VH comprising the amino acid sequence of SEQ ID NO: 1157 and a VL comprising the amino acid sequence of SEQ ID NO: 1158; and a second binding domain that binds to LILRB2 comprising a VH comprising the amino acid sequence of SEQ ID NO: 1177 and a VL comprising the amino acid sequence of SEQ ID NO: 1178. In some embodiments, provided herein is a multispecific binding agent comprising a first binding domain that binds to LILRB1 comprising a VH having at least 90% sequence identity to SEQ ID NO: 1157 and a VL having at least 90% sequence identity to SEQ ID NO: 1158; and a second binding domain that binds to LILRB2 comprising a VH having at least 90% sequence identity to SEQ ID NO: 1177 and a VL having at least 90% sequence identity to SEQ ID NO: 1178

In some embodiments, provided herein is a multispecific binding agent comprising a first binding domain that binds to LILRB1 comprising a VH comprising the amino acid sequence of SEQ ID NO: 1159 and a VL comprising the amino acid sequence of SEQ ID NO: 1160; and a second binding domain that binds to LILRB2 comprising a VH comprising the amino acid sequence of SEQ ID NO: 1167 and a VL comprising the amino acid sequence of SEQ ID NO: 1168. In some embodiments, provided herein is a multispecific binding agent comprising a first binding domain that binds to LILRB1 comprising a VH having at least 90% sequence identity to SEQ ID NO: 1159 and a VL having at least 90% sequence identity to SEQ ID NO: 1160; and a second binding domain that binds to LILRB2 comprising a VH having at least 90% sequence identity to SEQ ID NO: 1167 and a VL having at least 90% sequence identity to SEQ ID NO: 1168.

In some embodiments, provided herein is a multispecific binding agent comprising a first binding domain that binds to LILRB1 comprising a VH comprising the amino acid sequence of SEQ ID NO: 1159 and a VL comprising the amino acid sequence of SEQ ID NO: 1160; and a second binding domain that binds to LILRB2 comprising a VH comprising the amino acid sequence of SEQ ID NO: 1169 and a VL comprising the amino acid sequence of SEQ ID NO: 1170. In some embodiments, provided herein is a multispecific binding agent comprising a first binding domain that binds to LILRB1 comprising a VH having at least 90% sequence identity to SEQ ID NO: 1159 and a VL having at least 90% sequence identity to SEQ ID NO: 1160; and a second binding domain that binds to LILRB2 comprising a VH having at least 90% sequence identity to SEQ ID NO: 1169 and a VL having at least 90% sequence identity to SEQ ID NO: 1170.

In some embodiments, provided herein is a multispecific binding agent comprising a first binding domain that binds to LILRB1 comprising a VH comprising the amino acid sequence of SEQ ID NO: 1159 and a VL comprising the amino acid sequence of SEQ ID NO: 1160; and a second binding domain that binds to LILRB2 comprising a VH comprising the amino acid sequence of SEQ ID NO: 1171 and a VL comprising the amino acid sequence of SEQ ID NO: 1172. In some embodiments, provided herein is a multispecific binding agent comprising a first binding domain that binds to LILRB1 comprising a VH having at least 90% sequence identity to SEQ ID NO: 1159 and a VL having at least 90% sequence identity to SEQ ID NO: 1160; and a second binding domain that binds to LILRB2 comprising a VH having at least 90% sequence identity to SEQ ID NO: 1171 and a VL having at least 90% sequence identity to SEQ ID NO: 1172.

In some embodiments, provided herein is a multispecific binding agent comprising a first binding domain that binds to LILRB1 comprising a VH comprising the amino acid sequence of SEQ ID NO: 1159 and a VL comprising the amino acid sequence of SEQ ID NO: 1160; and a second binding domain that binds to LILRB2 comprising a VH comprising the amino acid sequence of SEQ ID NO: 1173 and a VL comprising the amino acid sequence of SEQ ID NO: 1174. In some embodiments, provided herein is a multispecific binding agent comprising a first binding domain that binds to LILRB1 comprising a VH having at least 90% sequence identity to SEQ ID NO: 1159 and a VL having at least 90% sequence identity to SEQ ID NO: 1160; and a second binding domain that binds to LILRB2 comprising a VH having at least 90% sequence identity to SEQ ID NO: 1173 and a VL having at least 90% sequence identity to SEQ ID NO: 1174.

In some embodiments, provided herein is a multispecific binding agent comprising a first binding domain that binds to LILRB1 comprising a VH comprising the amino acid sequence of SEQ ID NO: 1159 and a VL comprising the amino acid sequence of SEQ ID NO: 1160; and a second binding domain that binds to LILRB2 comprising a VH comprising the amino acid sequence of SEQ ID NO: 1175 and a VL comprising the amino acid sequence of SEQ ID NO: 1176. In some embodiments, provided herein is a multispecific binding agent comprising a first binding domain that binds to LILRB1 comprising a VH having at least 90% sequence identity to SEQ ID NO: 1159 and a VL having at least 90% sequence identity to SEQ ID NO: 1160; and a second binding domain that binds to LILRB2 comprising a VH having at least 90% sequence identity to SEQ ID NO: 1175 and a VL having at least 90% sequence identity to SEQ ID NO: 1176.

In some embodiments, provided herein is a multispecific binding agent comprising a first binding domain that binds to LILRB1 comprising a VH comprising the amino acid sequence of SEQ ID NO: 1159 and a VL comprising the amino acid sequence of SEQ ID NO: 1160; and a second binding domain that binds to LILRB2 comprising a VH comprising the amino acid sequence of SEQ ID NO: 1177 and a VL comprising the amino acid sequence of SEQ ID NO: 1178. In some embodiments, provided herein is a multispecific binding agent comprising a first binding domain that binds to LILRB1 comprising a VH having at least 90% sequence identity to SEQ ID NO: 1159 and a VL having at least 90% sequence identity to SEQ ID NO: 1160; and a second binding domain that binds to LILRB2 comprising a VH having at least 90% sequence identity to SEQ ID NO: 1177 and a VL having at least 90% sequence identity to SEQ ID NO: 1178.

In some embodiments, provided herein is a multispecific binding agent comprising a first binding domain that binds to LILRB1 comprising a VH comprising the amino acid sequence of SEQ ID NO: 1161 and a VL comprising the amino acid sequence of SEQ ID NO: 1162; and a second binding domain that binds to LILRB2 comprising a VH comprising the amino acid sequence of SEQ ID NO: 1167 and a VL comprising the amino acid sequence of SEQ ID NO: 1168. In some embodiments, provided herein is a multispecific binding agent comprising a first binding domain that binds to LILRB1 comprising a VH having at least 90% sequence identity to SEQ ID NO: 1161 and a VL having at least 90% sequence identity to SEQ ID NO: 1162; and a second binding domain that binds to LILRB2 comprising a VH having at least 90% sequence identity to SEQ ID NO: 1167 and a VL having at least 90% sequence identity to SEQ ID NO: 1168.

In some embodiments, provided herein is a multispecific binding agent comprising a first binding domain that binds to LILRB1 comprising a VH comprising the amino acid sequence of SEQ ID NO: 1161 and a VL comprising the amino acid sequence of SEQ ID NO: 1162; and a second binding domain that binds to LILRB2 comprising a VH comprising the amino acid sequence of SEQ ID NO: 1169 and a VL comprising the amino acid sequence of SEQ ID NO: 1170. In some embodiments, provided herein is a multispecific binding agent comprising a first binding domain that binds to LILRB1 comprising a VH having at least 90% sequence identity to SEQ ID NO: 1161 and a VL having at least 90% sequence identity to SEQ ID NO: 1162; and a second binding domain that binds to LILRB2 comprising a VH having at least 90% sequence identity to SEQ ID NO: 1169 and a VL having at least 90% sequence identity to SEQ ID NO: 1170.

In some embodiments, provided herein is a multispecific binding agent comprising a first binding domain that binds to LILRB1 comprising a VH comprising the amino acid sequence of SEQ ID NO: 1161 and a VL comprising the amino acid sequence of SEQ ID NO: 1162; and a second binding domain that binds to LILRB2 comprising a VH comprising the amino acid sequence of SEQ ID NO: 1171 and a VL comprising the amino acid sequence of SEQ ID NO: 1172. In some embodiments, provided herein is a multispecific binding agent comprising a first binding domain that binds to LILRB1 comprising a VH having at least 90% sequence identity to SEQ ID NO: 1161 and a VL having at least 90% sequence identity to SEQ ID NO: 1162; and a second binding domain that binds to LILRB2 comprising a VH having at least 90% sequence identity to SEQ ID NO: 1171 and a VL having at least 90% sequence identity to SEQ ID NO: 1172.

In some embodiments, provided herein is a multispecific binding agent comprising a first binding domain that binds to LILRB1 comprising a VH comprising the amino acid sequence of SEQ ID NO: 1161 and a VL comprising the amino acid sequence of SEQ ID NO: 1162; and a second binding domain that binds to LILRB2 comprising a VH comprising the amino acid sequence of SEQ ID NO: 1173 and a VL comprising the amino acid sequence of SEQ ID NO: 1174. In some embodiments, provided herein is a multispecific binding agent comprising a first binding domain that binds to LILRB1 comprising a VH having at least 90% sequence identity to SEQ ID NO: 1161 and a VL having at least 90% sequence identity to SEQ ID NO: 1162; and a second binding domain that binds to LILRB2 comprising a VH having at least 90% sequence identity to SEQ ID NO: 1173 and a VL having at least 90% sequence identity to SEQ ID NO: 1174.

In some embodiments, provided herein is a multispecific binding agent comprising a first binding domain that binds to LILRB1 comprising a VH comprising the amino acid sequence of SEQ ID NO: 1161 and a VL comprising the amino acid sequence of SEQ ID NO: 1162; and a second binding domain that binds to LILRB2 comprising a VH comprising the amino acid sequence of SEQ ID NO: 1175 and a VL comprising the amino acid sequence of SEQ ID NO: 1176. In some embodiments, provided herein is a multispecific binding agent comprising a first binding domain that binds to LILRB1 comprising a VH having at least 90% sequence identity to SEQ ID NO: 1161 and a VL having at least 90% sequence identity to SEQ ID NO: 1162; and a second binding domain that binds to LILRB2 comprising a VH having at least 90% sequence identity to SEQ ID NO: 1175 and a VL having at least 90% sequence identity to SEQ ID NO: 1176.

In some embodiments, provided herein is a multispecific binding agent comprising a first binding domain that binds to LILRB1 comprising a VH comprising the amino acid sequence of SEQ ID NO: 1161 and a VL comprising the amino acid sequence of SEQ ID NO: 1162; and a second binding domain that binds to LILRB2 comprising a VH comprising the amino acid sequence of SEQ ID NO: 1177 and a VL comprising the amino acid sequence of SEQ ID NO: 1178. In some embodiments, provided herein is a multispecific binding agent comprising a first binding domain that binds to LILRB1 comprising a VH having at least 90% sequence identity to SEQ ID NO: 1161 and a VL having at least 90% sequence identity to SEQ ID NO: 1162; and a second binding domain that binds to LILRB2 comprising a VH having at least 90% sequence identity to SEQ ID NO: 1177 and a VL having at least 90% sequence identity to SEQ ID NO: 1178.

In some embodiments, provided herein is a multispecific binding agent comprising a first binding domain that binds to LILRB1 comprising a VH comprising the amino acid sequence of SEQ ID NO: 1163 and a VL comprising the amino acid sequence of SEQ ID NO: 1164; and a second binding domain that binds to LILRB2 comprising a VH comprising the amino acid sequence of SEQ ID NO: 1167 and a VL comprising the amino acid sequence of SEQ ID NO: 1168. In some embodiments, provided herein is a multispecific binding agent comprising a first binding domain that binds to LILRB1 comprising a VH having at least 90% sequence identity to SEQ ID NO: 1163 and a VL having at least 90% sequence identity to SEQ ID NO: 1164; and a second binding domain that binds to LILRB2 comprising a VH having at least 90% sequence identity to SEQ ID NO: 1167 and a VL having at least 90% sequence identity to SEQ ID NO: 1168.

In some embodiments, provided herein is a multispecific binding agent comprising a first binding domain that binds to LILRB1 comprising a VH comprising the amino acid sequence of SEQ ID NO: 1163 and a VL comprising the amino acid sequence of SEQ ID NO: 1164; and a second binding domain that binds to LILRB2 comprising a VH comprising the amino acid sequence of SEQ ID NO: 1169 and a VL comprising the amino acid sequence of SEQ ID NO: 1170. In some embodiments, provided herein is a multispecific binding agent comprising a first binding domain that binds to LILRB1 comprising a VH having at least 90% sequence identity to SEQ ID NO: 1163 and a VL having at least 90% sequence identity to SEQ ID NO: 1164; and a second binding domain that binds to LILRB2 comprising a VH having at least 90% sequence identity to SEQ ID NO: 1169 and a VL having at least 90% sequence identity to SEQ ID NO: 1170.

In some embodiments, provided herein is a multispecific binding agent comprising a first binding domain that binds to LILRB1 comprising a VH comprising the amino acid sequence of SEQ ID NO: 1163 and a VL comprising the amino acid sequence of SEQ ID NO: 1164; and a second binding domain that binds to LILRB2 comprising a VH comprising the amino acid sequence of SEQ ID NO: 1171 and a VL comprising the amino acid sequence of SEQ ID NO: 1172. In some embodiments, provided herein is a multispecific binding agent comprising a first binding domain that binds to LILRB1 comprising a VH having at least 90% sequence identity to SEQ ID NO: 1163 and a VL having at least 90% sequence identity to SEQ ID NO: 1164; and a second binding domain that binds to LILRB2 comprising a VH having at least 90% sequence identity to SEQ ID NO: 1171 and a VL having at least 90% sequence identity to SEQ ID NO: 1172.

In some embodiments, provided herein is a multispecific binding agent comprising a first binding domain that binds to LILRB1 comprising a VH comprising the amino acid sequence of SEQ ID NO: 1163 and a VL comprising the amino acid sequence of SEQ ID NO: 1164; and a second binding domain that binds to LILRB2 comprising a VH comprising the amino acid sequence of SEQ ID NO: 1173 and a VL comprising the amino acid sequence of SEQ ID NO: 1174. In some embodiments, provided herein is a multispecific binding agent comprising a first binding domain that binds to LILRB1 comprising a VH having at least 90% sequence identity to SEQ ID NO: 1163 and a VL having at least 90% sequence identity to SEQ ID NO: 1164; and a second binding domain that binds to LILRB2 comprising a VH having at least 90% sequence identity to SEQ ID NO: 1173 and a VL having at least 90% sequence identity to SEQ ID NO: 1174.

In some embodiments, provided herein is a multispecific binding agent comprising a first binding domain that binds to LILRB1 comprising a VH comprising the amino acid sequence of SEQ ID NO: 1163 and a VL comprising the amino acid sequence of SEQ ID NO: 1164; and a second binding domain that binds to LILRB2 comprising a VH comprising the amino acid sequence of SEQ ID NO: 1175 and a VL comprising the amino acid sequence of SEQ ID NO: 1176. In some embodiments, provided herein is a multispecific binding agent comprising a first binding domain that binds to LILRB1 comprising a VH having at least 90% sequence identity to SEQ ID NO: 1163 and a VL having at least 90% sequence identity to SEQ ID NO: 1164; and a second binding domain that binds to LILRB2 comprising a VH having at least 90% sequence identity to SEQ ID NO: 1175 and a VL having at least 90% sequence identity to SEQ ID NO: 1176.

In some embodiments, provided herein is a multispecific binding agent comprising a first binding domain that binds to LILRB1 comprising a VH comprising the amino acid sequence of SEQ ID NO: 1163 and a VL comprising the amino acid sequence of SEQ ID NO: 1164; and a second binding domain that binds to LILRB2 comprising a VH comprising the amino acid sequence of SEQ ID NO: 1177 and a VL comprising the amino acid sequence of SEQ ID NO: 1178. In some embodiments, provided herein is a multispecific binding agent comprising a first binding domain that binds to LILRB1 comprising a VH having at least 90% sequence identity to SEQ ID NO: 1163 and a VL having at least 90% sequence identity to SEQ ID NO: 1164; and a second binding domain that binds to LILRB2 comprising a VH having at least 90% sequence identity to SEQ ID NO: 1177 and a VL having at least 90% sequence identity to SEQ ID NO: 1178.

In some embodiments, provided herein is a multispecific binding agent comprising a first binding domain that binds to LILRB1 comprising a VH comprising the amino acid sequence of SEQ ID NO: 1165 and a VL comprising the amino acid sequence of SEQ ID NO: 1166; and a second binding domain that binds to LILRB2 comprising a VH comprising the amino acid sequence of SEQ ID NO: 1167 and a VL comprising the amino acid sequence of SEQ ID NO: 1168. In some embodiments, provided herein is a multispecific binding agent comprising a first binding domain that binds to LILRB1 comprising a VH having at least 90% sequence identity to SEQ ID NO: 1165 and a VL having at least 90% sequence identity to SEQ ID NO: 1166; and a second binding domain that binds to LILRB2 comprising a VH having at least 90% sequence identity to SEQ ID NO: 1167 and a VL having at least 90% sequence identity to SEQ ID NO: 1168.

In some embodiments, provided herein is a multispecific binding agent comprising a first binding domain that binds to LILRB1 comprising a VH comprising the amino acid sequence of SEQ ID NO: 1165 and a VL comprising the amino acid sequence of SEQ ID NO: 1166; and a second binding domain that binds to LILRB2 comprising a VH comprising the amino acid sequence of SEQ ID NO: 1169 and a VL comprising the amino acid sequence of SEQ ID NO: 1170. In some embodiments, provided herein is a multispecific binding agent comprising a first binding domain that binds to LILRB1 comprising a VH having at least 90% sequence identity to SEQ ID NO: 1165 and a VL having at least 90% sequence identity to SEQ ID NO: 1166; and a second binding domain that binds to LILRB2 comprising a VH having at least 90% sequence identity to SEQ ID NO: 1169 and a VL having at least 90% sequence identity to SEQ ID NO: 1170.

In some embodiments, provided herein is a multispecific binding agent comprising a first binding domain that binds to LILRB1 comprising a VH comprising the amino acid sequence of SEQ ID NO: 1165 and a VL comprising the amino acid sequence of SEQ ID NO: 1166; and a second binding domain that binds to LILRB2 comprising a VH comprising the amino acid sequence of SEQ ID NO: 1171 and a VL comprising the amino acid sequence of SEQ ID NO: 1172. In some embodiments, provided herein is a multispecific binding agent comprising a first binding domain that binds to LILRB1 comprising a VH having at least 90% sequence identity to SEQ ID NO: 1165 and a VL having at least 90% sequence identity to SEQ ID NO: 1166; and a second binding domain that binds to LILRB2 comprising a VH having at least 90% sequence identity to SEQ ID NO: 1171 and a VL having at least 90% sequence identity to SEQ ID NO: 1172.

In some embodiments, provided herein is a multispecific binding agent comprising a first binding domain that binds to LILRB1 comprising a VH comprising the amino acid sequence of SEQ ID NO: 1165 and a VL comprising the amino acid sequence of SEQ ID NO: 1166; and a second binding domain that binds to LILRB2 comprising a VH comprising the amino acid sequence of SEQ ID NO: 1173 and a VL comprising the amino acid sequence of SEQ ID NO: 1174. In some embodiments, provided herein is a multispecific binding agent comprising a first binding domain that binds to LILRB1 comprising a VH having at least 90% sequence identity to SEQ ID NO: 1165 and a VL having at least 90% sequence identity to SEQ ID NO: 1166; and a second binding domain that binds to LILRB2 comprising a VH having at least 90% sequence identity to SEQ ID NO: 1173 and a VL having at least 90% sequence identity to SEQ ID NO: 1174.

In some embodiments, provided herein is a multispecific binding agent comprising a first binding domain that binds to LILRB1 comprising a VH comprising the amino acid sequence of SEQ ID NO: 1165 and a VL comprising the amino acid sequence of SEQ ID NO: 1166; and a second binding domain that binds to LILRB2 comprising a VH comprising the amino acid sequence of SEQ ID NO: 1175 and a VL comprising the amino acid sequence of SEQ ID NO: 1176. In some embodiments, provided herein is a multispecific binding agent comprising a first binding domain that binds to LILRB1 comprising a VH having at least 90% sequence identity to SEQ ID NO: 1165 and a VL having at least 90% sequence identity to SEQ ID NO: 1166; and a second binding domain that binds to LILRB2 comprising a VH having at least 90% sequence identity to SEQ ID NO: 1175 and a VL having at least 90% sequence identity to SEQ ID NO: 1176.

In some embodiments, provided herein is a multispecific binding agent comprising a first binding domain that binds to LILRB1 comprising a VH comprising the amino acid sequence of SEQ ID NO: 1165 and a VL comprising the amino acid sequence of SEQ ID NO: 1166; and a second binding domain that binds to LILRB2 comprising a VH comprising the amino acid sequence of SEQ ID NO: 1177 and a VL comprising the amino acid sequence of SEQ ID NO: 1178. In some embodiments, provided herein is a multispecific binding agent comprising a first binding domain that binds to LILRB1 comprising a VH having at least 90% sequence identity to SEQ ID NO: 1165 and a VL having at least 90% sequence identity to SEQ ID NO: 1166; and a second binding domain that binds to LILRB2 comprising a VH having at least 90% sequence identity to SEQ ID NO: 1177 and a VL having at least 90% sequence identity to SEQ ID NO: 1178.

In some embodiments, the multispecific binding agent provided herein comprises one LILRB1 binding domain (e.g., with a pair of VH and VL regions that bind to LILRB1) and one LILRB2 binding domain (e.g., with a pair of VH and VL regions that bind to LILRB2). In some embodiments, the multispecific binding agent provided herein comprises two LILRB1 binding domains (e.g., with two identical pairs of VH and VL regions that bind to LILRB1) and one LILRB2 binding domain (e.g., with a pair of VH and VL regions that bind to LILRB2). In some embodiments, the multispecific binding agent provided herein comprises one LILRB1 binding domain (e.g., with a pair of VH and VL regions that bind to LILRB1) and two LILRB2 binding domains (e.g., with two identical pairs of VH and VL regions that bind to LILRB2). In some embodiments, the multispecific binding agent provided herein comprises two LILRB1 binding domains (e.g., with two identical or different pair of VH and VL regions that bind to LILRB1) and two LILRB2 binding domains (e.g., with two identical or different pairs of VH and VL regions that bind to LILRB2. In some embodiments, the multispecific binding agent further comprises one or more heavy chain constant regions (such as CH1, CH2, and/or CH3) and/or one or more light chain constant regions (such as CL).

Exemplary structures of multispecific antibodies are known in the art and are further described in Weidle et al., 2013, Cancer Genomics & Proteomics 10:1-18; Brinkman et al., 2017, MABS, 9:2, 182-212; Godar et al., 2018, Expert Opinion on Therapeutic Patents, 28:3, 251-276; and Spiess et al., 2015, Mol. Immunol. 67 95-106.

For example, bispecific antibody molecules can be classified into different structural groups: (i) bispecific immunoglobulin G (BsIgG); (ii) IgG appended with an additional antigen-binding moiety; (iii) bispecific antibody fragments; (iv) bispecific fusion proteins; and (v) bispecific antibody conjugates. As a non-limiting example, BsIgG formats can include crossMab, DAF (two-in-one), DAF (four-in-one), DutaMab, DT-IgG, knobs-in-holes common LC, knobs-in-holes assembly, charge pair, Fab-arm exchange, SEEDbody, triomab, LUZ-Y, Fcab, KA-body, and/or orthogonal Fab.

In some embodiments, BsIgG comprises heavy chains that are engineered for heterodimerization. For example, heavy chains can be engineered for heterodimerization using a “knobs-into-holes” strategy, a SEED platform, a common heavy chain (e.g., in κλ-bodies), and use of heterodimeric Fc regions. Strategies are known in the art to avoid heavy chain pairing of homodimers in BsIgG, including knobs-into-holes, duobody, azymetric, charge pair, HA-TF, SEEDbody, and differential protein A affinity.

Another bispecific antibody format is IgG appended with an additional antigen-binding moiety. For example, monospecific IgG can be engineered to have bispecificity by appending an additional antigen-binding unit onto the monospecific IgG, e.g., at the N- or C-terminus of either the heavy or light chain. Exemplary additional antigen-binding units include single domain antibodies (e.g., variable heavy chain or variable light chain), engineered protein scaffolds, and paired antibody variable domains (e.g., single chain variable fragments or variable fragments). Non-limiting examples of appended IgG formats include dual variable domain IgG(DVD-Ig), IgG(H)-scFv, scFv-(H) IgG, IgG(L)-scFv, scFv-(L) IgG, IgG(L,H)-Fv, IgG(H)-V, V(H)-IgG, IgG(L)-V, V(L)-IgG, KIH IgG-scFab, 2scFv-IgG, IgG-2scFv, scFv4-Ig, zybody, and DVI-IgG(four-in-one). See Spiess et al. Mol. Immunol. 67 (2015): 95-106. In some embodiments, an exemplary antibody format is a B-Body format for monospecific or multispecific (e.g., bispecific antibodies) as described in e.g. WO 2018/075692, U.S. Pat. Publ. No. 2018/0118811, and U.S. Pat. Publ. No. 2021/0155692.

Bispecific (Bs) antibody (BsAb) fragments are a format of bispecific antibody molecules that lack some or all of the antibody constant domains. For example, some BsAb lack an Fc region. In embodiments, bispecific antibody fragments include heavy and light chain regions that are connected by a peptide linker that permits efficient expression of the BsAb in a single host cell. Non-limiting examples of bispecific antibody fragments include, but are not limited to, nanobody, nanobody-HAS, BiTE, Diabody, DART, TandAb, scDiabody, scDiabody-CH3, Diabody-CH3, triple body, miniantibody, minibody, TriBi minibody, scFv-CH3 KIH, Fab-scFv, scFv-CH-CL-scFv, F(ab′)₂, F(ab′)₂-scFv2, scFv-KIH, Fab-scFv-Fc, tetravalent HCAb, scDiabody-Fc, Diabody-Fc, tandem scFv-Fc, and intrabody.

In some embodiments, the multispecific binding agent provided herein has an antibody format of immunoglobulin-single chain antibody fragment (IgG-ScFv). “ScFv” are antibody fragments that comprise the VH and VL antibody domains connected into a single polypeptide chain. Preferably, the scFv polypeptide further comprises a polypeptide linker between the VH and VL domains which enables the sFv to form the desired structure for antigen binding. In some embodiments, the heavy chain of the IgG is linked to one or more scFv. In some embodiments, the light chain of the IgG is linked to one or more scFv. In some embodiments, the scFv is linked to the C terminal of the heavy chain of the IgG. In some embodiments, the scFv is linked to the N terminal of the heavy chain of the IgG. In some embodiments, the scFv is linked to the C terminal of the light chain of the IgG. In some embodiments, the scFv is linked to the N terminal of the light chain of the IgG. In some embodiments, one of heavy chains of the IgG is linked to scFv. In some embodiments, both heavy chains of the IgG are linked to scFv. In some embodiments, one of light chains of the IgG is linked to scFv. In some embodiments, both light chains of the IgG are linked to scFv. In some embodiments, the scFv domain is in the VL-VH orientation from N terminal to C terminal. In some embodiments the scFv domain is in the VH-VL orientation from N terminal to C terminal. In some embodiments, the scFv domain binds to LILRB1 and the IgG domain binds to LILRB2. In some embodiments, the scFv domain binds to LILRB2 and the IgG domain binds to LILRB1.

In some embodiments, the multispecific binding agent provided herein has an antibody format of dual-variable-domain-immunoglobulin (DVD-Ig). The dual variable domain immunoglobulin (DVD-Ig) molecule is designed such that two different light chain variable domains (VL) are linked in tandem directly or via a short linker, followed by the light chain constant domain. Similarly, the heavy chain comprises two different heavy chain variable domains (VH) linked in tandem directly or via a short linker, followed by the constant domain CHI and Fc region. In some embodiments, the N-terminal variable domain VH and VL bind to LILRB1 and the VH and VL proximal to the C-terminal CH1 or CL bind to LILRB2. In some embodiments, the N-terminal variable domain VH and VL bind to LILRB2 and the VH and VL proximal to the C-terminal CH1 or CL bind to LILRB1.

Although specific exemplary multispecific antibody formats are described in the Example section below, it is contemplated that any multispecific antibody formats known in the art can be used and are included in the present disclosure.

Multispecific binding agents (e.g., bispecific antibodies) described herein may be bispecific, trispecific or of greater multispecificity. Such agents may include multispecific antibodies. In some embodiments, multispecific antibodies, such as bispecific antibodies, are monoclonal antibodies that have binding specificities for at least two different targets (e.g., antigens) or two different epitopes on the same target (e.g., a bispecific antibody directed to LILRB1 with a first binding domain for a first epitope of an LILRB1, and a second binding domain for a second epitope of LILRB1). In some embodiments, the multispecific (e.g., bispecific) antibodies can be constructed based on the sequences of the antibodies described herein, e.g., the CDR sequences listed in Tables 5-6, and 14-15. In some embodiments, the multispecific antibodies described herein are bispecific antibodies. In some embodiments, the multispecific antibody described herein (e.g., a bispecific antibody) has binding specificities for two targets, for example, LILRB1 and LILRB2. In some embodiments, bispecific antibodies are mouse, chimeric, human or humanized antibodies.

In some embodiments, one of the binding specificities of the multispecific antibody provided herein is for LILRB1, a second binding specificity of the multispecific antibody provided herein is LILRB2, and yet one or more additional binding specificities are for any other target(s) (e.g., antigen). In some embodiments, a multispecific antibody can comprise more than one target (e.g., antigen) binding domain, in which different binding domains are specific for different targets. In some embodiments, the additional target is an immune checkpoint regulator (e.g., a negative checkpoint regulator). In some embodiments, the additional target is expressed on an immune cell. In some embodiments, the additional target is expressed on a tumor or cancer cell.

In some embodiments, multispecific (e.g., bispecific) antibody molecules can bind more than one (e.g., two or more) epitopes on the same target (e.g., antigen). In some embodiments, a multispecific (e.g., bispecific) binder as disclosed herein can bind to one or more epitopes on a first target (such as LILRB1) and one or more epitopes on a second target (such as LILRB2).

Methods for making multispecific antibodies are known in the art, such as, by co-expression of two immunoglobulin heavy chain-light chain pairs, where the two heavy chains have different specificities (see, e.g., Milstein and Cuello, 1983, Nature 305:537-40). For further details of generating multispecific antibodies (e.g., bispecific antibodies), see, for example, Bispecific Antibodies (Kontermann ed., 2011).

Bispecific fusion proteins include antibody fragments linked to other proteins. For example, bispecific fusion proteins can be linked to other proteins to add additional specificity and/or functionality. In some embodiments, the dock-and-lock (DNL) method can be used to generate bispecific antibody molecules with higher valency. For example, bispecific antibody fusions to albumin binding proteins or human serum albumin can be extend the serum half-life of antibody fragments. In embodiments, chemical conjugation, e.g., chemical conjugation of antibodies and/or antibody fragments, can be used to create BsAb molecules. An exemplary bispecific antibody conjugate includes the CovX-body format, in which a low molecular weight drug is conjugated site-specifically to a single reactive lysine in each Fab arm or an antibody or fragment thereof. In some embodiments, the conjugation improves the serum half-life.

Methods of production of multispecific antibodies, including bispecific antibodies, are known in the art. For example, multispecific antibodies, including bispecific antibodies, can be produced by separate expression of the component antibodies in different host cells and subsequent purification/assembly or by expression of the component antibodies in a single host cell. Purification of multispecific (e.g., bispecific) antibody molecules can be performed by various methods known in the art, including affinity chromatography.

In some embodiments, multispecific binding agents (e.g., bispecific antibodies) disclosed herein can be provided in any antibody format disclosed herein or known in the art. As a non-limiting example, in some embodiments, the multispecific binding agents (e.g., bispecific antibodies) can be selected from Fabs-in-tandem-lg (FIT-1g); DVD-1g; hybrid hybridoma (quadroma or tetradoma); anticalin platform (Pieris); diabodies; single chain diabodies; tandem single chain Fv fragments; TandAbs, Trispecific Abs (Affimed); Darts dual affinity retargeting (Macrogenics); Bispecific Xmabs (Xencor); Bispecific T cell engagers (Bites; Amgen; 55 kDa); Triplebodies; Tribody=Fab-scFv Fusion Protein multifunctional recombinant antibody derivates (CreativeBiolabs); Duobody platform (Genmab); dock and lock platform; knobs-into-holes (KIH) platform; Humanized bispecific IgG antibody (REGN1979) (Regeneron); Mab2 bispecific antibodies (F-Star); DVD-lg=dual variable domain immunoglobulin (Abbott); kappa-lambda bodies; TBTI=tetravalent bispecific tandem Ig; B-Body; and CrossMab (Roche).

In some embodiments, the multispecific binding agent provided herein (e.g., a bispecific antibody) comprises a first polypeptide comprising the amino acid sequence of SEQ ID NO: 1180, and a second polypeptide comprising the amino acid sequence of SEQ ID NO: 1181. In some embodiments, the multispecific binding agent provided herein (e.g., a bispecific antibody) comprises a first polypeptide comprising the amino acid sequence of SEQ ID NO: 1182, and a second polypeptide comprising the amino acid sequence of SEQ ID NO: 1183. In some embodiments, the multispecific binding agent provided herein (e.g., a bispecific antibody) comprises a first polypeptide comprising the amino acid sequence of SEQ ID NO: 1184, and a second polypeptide comprising the amino acid sequence of SEQ ID NO: 1185. In some embodiments, the multispecific binding agent provided herein (e.g., a bispecific antibody) comprises a first polypeptide comprising the amino acid sequence of SEQ ID NO: 1186, and a second polypeptide comprising the amino acid sequence of SEQ ID NO: 1187. In some embodiments, the multispecific binding agent provided herein (e.g., a bispecific antibody) comprises a first polypeptide comprising the amino acid sequence of SEQ ID NO: 1188, and a second polypeptide comprising the amino acid sequence of SEQ ID NO: 1189. In some embodiments, the multispecific binding agent provided herein (e.g., a bispecific antibody) comprises a first polypeptide comprising the amino acid sequence of SEQ ID NO: 1190, and a second polypeptide comprising the amino acid sequence of SEQ ID NO: 1191. In some embodiments, the multispecific binding agent provided herein (e.g., a bispecific antibody) comprises a first polypeptide comprising the amino acid sequence of SEQ ID NO: 1192, and a second polypeptide comprising the amino acid sequence of SEQ ID NO: 1193. In some embodiments, the multispecific binding agent provided herein (e.g., a bispecific antibody) comprises a first polypeptide comprising the amino acid sequence of SEQ ID NO: 1194, and a second polypeptide comprising the amino acid sequence of SEQ ID NO: 1195. In some embodiments, the multispecific binding agent provided herein (e.g., a bispecific antibody) comprises a first polypeptide comprising the amino acid sequence of SEQ ID NO: 1196, and a second polypeptide comprising the amino acid sequence of SEQ ID NO: 1197. In some embodiments, the multispecific binding agent provided herein (e.g., a bispecific antibody) comprises a first polypeptide comprising the amino acid sequence of SEQ ID NO: 1198, and a second polypeptide comprising the amino acid sequence of SEQ ID NO: 1199. In some embodiments, the multispecific binding agent provided herein (e.g., a bispecific antibody) comprises a first polypeptide comprising the amino acid sequence of SEQ ID NO: 1200, and a second polypeptide comprising the amino acid sequence of SEQ ID NO: 1201. In some embodiments, the multispecific binding agent provided herein (e.g., a bispecific antibody) comprises a first polypeptide comprising the amino acid sequence of SEQ ID NO: 1202, and a second polypeptide comprising the amino acid sequence of SEQ ID NO: 1203.

In some embodiments, the multispecific binding agent provided herein inhibits the LILRB1 signaling. In some embodiments, the multispecific binding agent provided herein inhibits the LILRB2 signaling.

In some embodiments, provided herein are multispecific antibodies that specifically bind to LILRB1 and can modulate LILRB1 activity and/or expression (e.g., inhibit LILRB1 mediated signaling). LILRB1 activity or LILRB1 signaling activity includes, but is not limited to, suppression of myeloid cells, suppression of myeloid cell activity, suppression of tumor-associated myeloid cells, suppression of NK cells, and suppression of cytolytic T-cells. In certain embodiments, a LILRB1 antagonist is provided herein that is an antibody described herein that specifically binds to LILRB1 and inhibits (including partially inhibits) at least one LILRB1 activity. In some embodiments, an anti-LILRB1 antibody inhibits LILRB1 signaling thereby reversing an LILRB1-induced suppressive effect. In some embodiments, the antibodies provided herein inhibit (including partially inhibit or reduce) the binding of LILRB1 to its ligands. In some embodiments, the ligands are MHC I molecules. In some embodiments, the ligand is HLA-A2. In some embodiments, the ligand is HLA-G. A LILRB1 activity can relate to any activity of LILRB1 such as those known or described in the art. In certain embodiments, LILRB1 activity and LILRB1 signaling (or LILRB1 mediated signaling) are used interchangeably herein.

In some embodiments, provided herein are multispecific antibodies that specifically bind to LILRB2 and can modulate LILRB2 activity and/or expression (e.g., inhibit LILRB2 mediated signaling). LILRB2 activity or LILRB2 signaling activity includes, but is not limited to, suppression of myeloid cells, suppression of myeloid cell activity, suppression of tumor-associated myeloid cells, suppression of NK cells, and suppression of cytolytic T-cells. In certain embodiments, a LILRB2 antagonist is provided herein that is an antibody described herein that specifically binds to LILRB2 and inhibits (including partially inhibits) at least one LILRB2 activity. In some embodiments, an anti-LILRB2 antibody inhibits LILRB2 signaling thereby reversing an LILRB2-induced suppressive effect. In some embodiments, the antibodies provided herein inhibit (including partially inhibit or reduce) the binding of LILRB2 to its ligands. In some embodiments, the ligands are MHC I molecules. In some embodiments, the ligand is HLA-A2. In some embodiments, the ligand is HLA-G. In some embodiments, the ligand is ANGPTLs. A LILRB2 activity can relate to any activity of LILRB2 such as those known or described in the art. In certain embodiments, LILRB2 activity and LILRB2 signaling (or LILRB2 mediated signaling) are used interchangeably herein.

In other embodiments, the antibody described herein can prevent suppression of an immune cell, e.g., a suppression mediated by the interaction between LILRB1 expressed on the immune cell with ligands of LILRB1 (e.g., expressed on a cancer or tumor cell) and the interaction between LILRB2 expressed on the immune cell with ligands of LILRB2 (e.g., expressed on a cancer or tumor cell). In some embodiments, the ligands are MHC I molecules. In some embodiments, the ligand is HLA-A2. In some embodiments, the ligand is HLA-G. In some embodiments, the ligand is ANGPTLs. In yet other embodiments, the antibody described herein can activate a response mediated by an immune cell, e.g., an anti-tumor response. In some embodiments, the immune cell is an NK cell. In some embodiments, the immune cell is a T cell. In some embodiments, the T cell is a cytotoxic T cell such as a CD8+ T cell. In some embodiments, the immune cell is a monocyte. In some embodiments, the immune cell is a macrophage. In some embodiments, the immune cell is a dendritic cell. In some embodiments, the immune cell mediates an anti-cancer/tumor response. In further embodiments, the cancer or tumor cell expresses HLA-A2. Additionally or alternatively, the cancer or tumor cell expresses HLA-G. In further embodiments, the cancer or tumor cell expresses ANGPTLs.

In some embodiments, the antibody described herein can increases myeloid cell activity. In some embodiments, the antibody described herein can enhance the activation of NK cells. In some embodiments, the antibody described herein can enhance macrophage activity. In some embodiments, the antibody described herein can enhance macrophage phagocytosis. In some embodiments, the antibody described herein can enhance T cell activity. In some embodiments, the antibody described herein can enhance the activity of CD8+ T cells. In some embodiments, the antibody described herein can enhance dendritic cell activity. In some embodiments, the antibody described herein can enhance FcR-mediated dendritic cell activity. In some embodiments, the antibody described herein can enhance cytokine release by immune cells. In some embodiments, the antibody described herein can enhance TNFα release by immune cells. In some embodiments, the antibody described herein can enhance IFN-γ release by immune cells.

In some embodiments, the antibody described herein enhances anti-tumor immunity by at least 10%. In some embodiments, the antibody described herein enhances anti-tumor immunity by at least 20%. In some embodiments, the antibody described herein enhances anti-tumor immunity by at least 30%. In some embodiments, the antibody described herein enhances anti-tumor immunity by at least 40%. In some embodiments, the antibody described herein enhances anti-tumor immunity by at least 50%. In some embodiments, the antibody described herein enhances anti-tumor immunity by at least 60%. In some embodiments, the antibody described herein enhances anti-tumor immunity by at least 70%. In some embodiments, the antibody described herein enhances anti-tumor immunity by at least 80%. In some embodiments, the antibody described herein enhances anti-tumor immunity by at least 90%. In some embodiments, the antibody described herein enhances anti-tumor immunity by at least 95%. In certain embodiments, the antibody described herein enhances anti-tumor immunity by at least about 15% to about 65%. In certain embodiments, the antibody described herein enhances anti-tumor immunity by at least about 20% to about 65%. In certain embodiments, the antibody described herein enhances anti-tumor immunity by at least about 30% to about 65%.

Other exemplary binding molecules are described in more detail in the following sections. In some embodiments, the multispecific binding agents according to any of the above embodiments may incorporate any of the features, singly or in combination, as described in Sections 5.2.4 to 5.2.7 below.

### 5.2.4. Antibody Fragments

As used herein, the term “antibody” also includes various antibody fragments thereof. Antibodies provided herein include, but are not limited to, immunoglobulin molecules and immunologically active portions of immunoglobulin molecules. The immunoglobulin molecules provided herein can be of any class (e.g., IgG, IgE, IgM, IgD, and IgA) or any subclass (e.g., IgG1, IgG2, IgG3, IgG4, IgA1, and IgA2) of immunoglobulin molecule. In some embodiments, the antibody is an IgG antibody. In some embodiments, the IgG antibody is an IgG1 antibody. In some embodiments, the IgG antibody is an IgG2, IgG3, or IgG4 antibody.

Variants and derivatives of antibodies include antibody functional fragments that retain the ability to bind to an antigen. Antibody fragments include but are not limited to those described in Section 5.1 above. Exemplary functional fragments include Fab fragments (e.g., an antibody fragment that contains the antigen-binding domain and comprises a light chain and part of a heavy chain bridged by a disulfide bond); Fab′ (e.g., an antibody fragment containing a single antigen-binding domain comprising an Fab and an additional portion of the heavy chain through the hinge region); F(ab′)₂ (e.g., two Fab′ molecules joined by interchain disulfide bonds in the hinge regions of the heavy chains; the Fab′ molecules may be directed toward the same or different epitopes); a bispecific Fab (e.g., a Fab molecule having two antigen binding domains, each of which may be directed to a different epitope); a single chain comprising a variable region, also known as, scFv (e.g., the variable, antigen-binding determinative region of a single light and heavy chain of an antibody linked together by a chain of, e.g., 10-25 amino acids); a disulfide-linked Fv, or dsFv (e.g., the variable, antigen-binding determinative region of a single light and heavy chain of an antibody linked together by a disulfide bond); a camelized VH (e.g., the variable, antigen-binding determinative region of a single heavy chain of an antibody in which some amino acids at the VH interface are those found in the heavy chain of naturally occurring camel antibodies); a bispecific scFv (e.g., an scFv or a dsFv molecule having two antigen-binding domains, each of which may be directed to a different epitope); a diabody (e.g., a dimerized scFv formed when the VH domain of a first scFv assembles with the VL domain of a second scFv and the VL domain of the first scFv assembles with the VH domain of the second scFv; the two antigen-binding regions of the diabody may be directed towards the same or different epitopes); a triabody (e.g., a trimerized scFv, formed in a manner similar to a diabody, but in which three antigen-binding domains are created in a single complex; the three antigen binding domains may be directed towards the same or different epitopes); and a tetrabody (e.g., a tetramerized scFv, formed in a manner similar to a diabody, but in which four antigen-binding domains are created in a single complex; the four antigen binding domains may be directed towards the same or different epitopes).

Various techniques have been developed for the production of antibody fragments. Traditionally, these fragments were derived via proteolytic digestion of intact antibodies (see, e.g., Morimoto et al., 1992, J. Biochem. Biophys. Methods 24:107-17; and Brennan et al., 1985, Science 229:81-83). However, these fragments can now be produced directly by recombinant host cells. For example, Fab, Fv, and scFv antibody fragments can all be expressed in and secreted from E. coli or yeast cells, thus allowing the facile production of large amounts of these fragments. Antibody fragments can be isolated from the antibody phage libraries discussed above. Alternatively, Fab′-SH fragments can be directly recovered from E. coli and chemically coupled to form F(ab′)₂ fragments (Carter et al., 1992, Bio/Technology 10:163-67). According to another approach, F(ab′)₂ fragments can be isolated directly from recombinant host cell culture. Fab and F(ab′)₂ fragment with increased in vivo half-life comprising salvage receptor binding epitope residues are described in, for example, U.S. Pat. No. 5,869,046. Other techniques for the production of antibody fragments will be apparent to the skilled practitioner. In certain embodiments, an antibody is a single chain Fv fragment (scFv) (see, e.g., WO 93/16185; U.S. Pat. Nos. 5,571,894 and 5,587,458). Fv and scFv have intact combining sites that are devoid of constant regions; thus, they may be suitable for reduced nonspecific binding during in vivo use. scFv fusion proteins may be constructed to yield fusion of an effector protein at either the amino or the carboxy terminus of an scFv (See, e.g., Borrebaeck ed., supra). The antibody fragment may also be a “linear antibody,” for example, as described in the references cited above. Such linear antibodies may be monospecific or multi-specific, such as bispecific.

### 5.2.5. Humanized Antibodies

The present disclosure provides humanized antibodies that bind to LILRB1 and/or LILRB2, including human LILRB1 and human LILRB2 (e.g. humanized anti-LILRB1 antibodies, humanized anti-LILRB2 antibodies, humanized multispecific antibodies that bind to both LILRB1 and LILRB2). Humanized antibodies of the present disclosure may comprise one or more CDRs from a VH and/or VL disclosed herein, such as those as shown in Tables 5-6, and 14-15. Various methods for humanizing non-human antibodies are known in the art. For example, a humanized antibody can have one or more amino acid residues introduced into it from a source that is non-human. These non-human amino acid residues are often referred to as “import” residues, which are typically taken from an “import” variable domain. Humanized antibodies that bind LILRB1 and/or LILRB2 may be produced using techniques known to those skilled in the art (Zhang et al., Molecular Immunology, 42 (12): 1445-1451, 2005; Hwang et al., Methods, 36 (1): 35-42, 2005; Dall'Acqua et al., Methods, 36 (1): 43-60, 2005; Clark, Immunology Today, 21 (8): 397-402, 2000, and U.S. Pat. Nos. 6,180,370; 6,054,927; 5,869,619; 5,861,155; 5,712,120; and 4,816,567).

In some cases, the humanized antibodies are constructed by CDR grafting, in which the amino acid sequences of the six CDRs of a VH and a VL of the parent non-human antibody (e.g., rodent) are grafted onto a human antibody framework. For example, Padlan et al. (FASEB J. 9:133-139, 1995) determined that only about one third of the residues in the CDRs actually contact the antigen, and termed these the “specificity determining residues,” or SDRs. In the technique of SDR grafting, only the SDR residues are grafted onto the human antibody framework (see, e.g., Kashmiri et al., Methods 36:25-34, 2005).

The choice of human variable domains, both light and heavy, to be used in making the humanized antibodies can be important to reduce antigenicity. For example, according to the so-called “best-fit” method, the sequence of the variable domain of a non-human (e.g., rodent) antibody is screened against the entire library of known human variable-domain sequences. The human sequence which is closest to that of the rodent may be selected as the human framework for the humanized antibody (Sims et al. (1993) J. Immunol. 151:2296; Chothia et al. (1987) J. Mol. Biol. 196:901). Another method uses a particular framework derived from the consensus sequence of all human antibodies of a particular subgroup of light or heavy chains. The same framework may be used for several different humanized antibodies (Carter et al. (1992) Proc. Natl. Acad. Sci. USA, 89:4285; Presta et al. (1993) J. Immunol., 151:2623). In some cases, the framework is derived from the consensus sequences of the most abundant human subclasses, V_{L}6 subgroup I (V_{L}6I) and V_{H} subgroup III (VHIII). In another method, human germline genes are used at the source of the framework regions.

In an alternative paradigm based on comparison of CDRs, called Superhumanization, framework homology is irrelevant. The method consists of comparison of the non-human sequence with the functional human germline gene repertoire. Those genes encoding the same or closely related canonical structures to the murine sequences are then selected. Next, within the genes sharing the canonical structures with the non-human antibody, those with highest homology within the CDRs are chosen as framework donors. Finally, the non-human CDRs are grafted onto these frameworks (see, e.g., Tan et al., J. Immunol. 169:1119-1125, 2002).

It is further generally desirable that antibodies be humanized with retention of their affinity for the antigen and other favorable biological properties. To achieve this goal, according to one method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. These include, for example, WAM (Whitelegg and Rees, Protein Eng. 13:819-824, 2000), Modeller (Sali and Blundell, J. Mol. Biol. 234:779-815, 1993), and Swiss PDB Viewer (Guex and Peitsch, Electrophoresis 18:2714-2713, 1997). Inspection of these displays permits analysis of the likely role of the residues in the functioning of the candidate immunoglobulin sequence, e.g., the analysis of residues that influence the ability of the candidate immunoglobulin to bind its antigen. In this way, framework residues can be selected and combined from the recipient and import sequences so that the desired antibody characteristic, such as increased affinity for the target antigen(s), is achieved. In general, the hypervariable region residues are directly and most substantially involved in influencing antigen binding.

Another method for antibody humanization is based on a metric of antibody humanness termed Human String Content (HSC). This method compares the mouse sequence with the repertoire of human germline genes and the differences are scored as HSC. The target sequence is then humanized by maximizing its HSC rather than using a global identity measure to generate multiple diverse humanized variants. See, e.g., Lazar et al., Mol. Immunol. 44:1986-1998, 2007.

In addition to the methods described above, empirical methods may be used to generate and select humanized antibodies. These methods include those that are based upon the generation of large libraries of humanized variants and selection of the best clones using enrichment technologies or high throughput screening techniques. Antibody variants may be isolated from phage, ribosome and yeast display libraries as well as by bacterial colony screening (see, e.g., Hoogenboom, Nat. Biotechnol. 23:1105-1116, 2005; Dufner et al., Trends Biotechnol. 24:523-529, 2006; Feldhaus et al., Nat. Biotechnol. 21:163-70, 2003; Schlapschy et al., Protein Eng. Des. Sel. 17:847-60, 2004).

In the framework library approach, a collection of residue variants are introduced at specific positions in the framework followed by selection of the library to select the framework that best supports the grafted CDR. The residues to be substituted may include some or all of the “Vernier” residues identified as potentially contributing to CDR structure (see, e.g., Foote and Winter, J. Mol. Biol. 224:487-499, 1992), or from the more limited set of target residues identified by Baca et al. (J. Biol. Chem. 272:10678-10684, 1997).

In framework shuffling, whole frameworks are combined with the non-human CDRs instead of creating combinatorial libraries of selected residue variants (see, e.g., Dall'Acqua et al., Methods 36:43-60, 2005). The libraries may be screened for binding in a two-step selection process, first humanizing VL, followed by VH. Alternatively, a one-step framework shuffling process may be used. Such a process has been shown to be more efficient than the two-step screening, as the resulting antibodies exhibited improved biochemical and physico-chemical properties including enhanced expression, increased affinity and thermal stability (see, e.g., Damschroder et al., Mol. Immunol. 44:3049-60, 2007).

The “humaneering” method is based on experimental identification of essential minimum specificity determinants (MSDs) and is based on sequential replacement of non-human fragments into libraries of human frameworks and assessment of binding. It begins with regions of the CDR3 of non-human VH and VL chains and progressively replaces other regions of the non-human antibody into the human frameworks, including the CDR1 and CDR2 of both VH and VL. This methodology typically results in epitope retention and identification of antibodies from multiple sub-classes with distinct human V-segment CDRs. Humaneering allows for isolation of antibodies that are 91-96% homologous to human germline gene antibodies. See, e.g., Alfenito, Cambridge Healthtech Institute's Third Annual PEGS, The Protein Engineering Summit, 2007.

The “human engineering” method involves altering a non-human antibody or antibody fragment, such as a mouse or chimeric antibody or antibody fragment, by making specific changes to the amino acid sequence of the antibody so as to produce a modified antibody with reduced immunogenicity in a human that nonetheless retains the desirable binding properties of the original non-human antibodies. Generally, the technique involves classifying amino acid residues of a non-human (e.g., mouse) antibody as “low risk”, “moderate risk”, or “high risk” residues. The classification is performed using a global risk/reward calculation that evaluates the predicted benefits of making a particular substitution (e.g., for immunogenicity in humans) against the risk that the substitution will affect the resulting antibody's folding and/or are substituted with human residues. The particular human amino acid residue to be substituted at a given position (e.g., low or moderate risk) of a non-human (e.g., mouse) antibody sequence can be selected by aligning an amino acid sequence from the non-human antibody's variable regions with the corresponding region of a specific or consensus human antibody sequence. The amino acid residues at low or moderate risk positions in the non-human sequence can be substituted for the corresponding residues in the human antibody sequence according to the alignment. Techniques for making human engineered proteins are described in greater detail in Studnicka et al., Protein Engineering, 7:805-814 (1994), U.S. Pat. Nos. 5,766,886, 5,770,196, 5,821,123, and 5,869,619, and WO 93/11794.

### 5.2.6. Antibody Variants

In some embodiments, amino acid sequence modification(s) of the antibodies that bind to LILRB1 and/or LILRB2 described herein (e.g. the anti-LILRB1 antibodies, the anti-LILRB2 antibodies, the multispecific antibodies that bind to both LILRB1 and LILRB2) are contemplated. For example, it may be desirable to optimize the binding affinity and/or other biological properties of the antibody, including but not limited to specificity, thermostability, expression level, effector functions, glycosylation, reduced immunogenicity, or solubility. Thus, it is contemplated that variants of the antibodies described herein can be prepared and are included in the present disclosure. In some embodiments, antibody variants are antibodies with amino acid sequence variations as compared with the original antibody, for example, substitution, deletion, or insertion of one or more amino acid(s), as described above. For example, variations may be a substitution, deletion, or insertion of one or more codons encoding the antibody or polypeptide that results in a change in the amino acid sequence (e.g., a conservative substitution) as compared with the original antibody or polypeptide. Sites of interest for substitutional mutagenesis include the CDRs, FRs and/or constant regions. For example, antibody variants can be prepared by introducing appropriate nucleotide changes into the encoding DNA, and/or by synthesis of the desired antibody or polypeptide. Those skilled in the art who appreciate that amino acid changes may alter post-translational processes of the antibody (e.g., the anti-LILRB1 antibodies, the anti-LILRB2 antibodies, the multispecific antibodies that bind to both LILRB1 and LILRB2).

### Chemical Modifications

Other exemplary modifications include chemical modifications, for example, by the covalent attachment of any type of molecule to the antibody provided herein. Antibody derivatives may include antibodies that have been chemically modified, for example, by glycosylation, acetylation, pegylation, phosphorylation, amidation, derivatization by known protecting/blocking groups, proteolytic cleavage, linkage to a cellular ligand or other protein, or conjugation to one or more immunoglobulin domains (e.g., Fc or a portion of an Fc). Any of numerous chemical modifications may be carried out by known techniques, including, but not limited to, specific chemical cleavage, acetylation, formulation, metabolic synthesis of tunicamycin, etc. Additionally, the antibody may contain one or more non-classical amino acids.

In some embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

When the antibody provided herein (e.g., the anti-LILRB1 antibodies, the anti-LILRB2 antibodies, the multispecific antibodies that bind to both LILRB1 and LILRB2) is fused to an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in the binding molecules provided herein may be made in order to create variants with certain improved properties.

In other embodiments, when the antibody provided herein is fused to an Fc region, antibody variants provided herein may have a carbohydrate structure that lacks fucose attached (directly or indirectly) to said Fc region. For example, the amount of fucose in such antibody may be from 1% to 80%, from 1% to 65%, from 5% to 65%, or from 20% to 40%. The amount of fucose is determined by calculating the average amount of fucose within the sugar chain at Asn297, relative to the sum of all glycostructures attached to Asn297 (e.g., complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues); however, Asn297 may also be located about +3 amino acids upstream or downstream of position 297, i.e., between positions 294 and 300, due to minor sequence variations in antibodies. Such fucosylation variants may have improved ADCC function. See, e.g., U.S. Pat. Publ. Nos. 2003/0157108 and 2004/0093621. Examples of publications related to “defucosylated” or “fucose-deficient” antibody variants include: U.S. Pat. Publ. No. 2003/0157108; WO 2000/61739; WO 2001/29246; U.S. Pat. Publ. No. 2003/0115614; U.S. Pat. Publ. No. 2002/0164328; U.S. Pat. Publ. No. 2004/0093621; U.S. Pat. Publ. No. 2004/0132140; U.S. Pat. Publ. No. 2004/0110704; U.S. Pat. Publ. No. 2004/0110282; U.S. Pat. Publ. No. 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87:614 (2004). Examples of cell lines capable of producing defucosylated antibodies include Lec13 CHO cells deficient in protein fucosylation (Ripka et al. Arch. Biochem. Biophys. 249:533-545 (1986); U.S. Pat. Publ. No. 2003/0157108; and WO 2004/056312, and knockout cell lines, such as alpha-1,6-fucosyltransferase gene, FUT8, knockout CHO cells (see, e.g., Yamane-Ohnuki et al. Biotech. Bioeng. 87:614 (2004); Kanda, Y. et al., Biotechnol. Bioeng., 94 (4): 680-688 (2006); and WO2003/085107).

The binding molecules comprising an antibody provided herein are further provided with bisected oligosaccharides, e.g., in which a biantennary oligosaccharide attached to the Fc region is bisected by GlcNAc. Such variants may have reduced fucosylation and/or improved ADCC function. Examples of such variants are described, e.g., in WO 2003/011878 (Jean-Mairet et al.); U.S. Pat. No. 6,602,684 (Umana et al.); and U.S. Pat. Publ. No. 2005/0123546 (Umana et al.). Variants with at least one galactose residue in the oligosaccharide attached to the Fc region are also provided. Such variants may have improved CDC function. Such variants are described, e.g., in WO 1997/30087; WO 1998/58964; and WO 1999/22764.

In molecules that comprise the present antibody and an Fc region, one or more amino acid modifications may be introduced into the Fc region, thereby generating an Fc region variant. The Fc region variant may comprise a human Fc region sequence (e.g., a human IgG1, IgG2, IgG3 or IgG4 Fc region) comprising an amino acid modification (e.g., a substitution) at one or more amino acid positions.

In some embodiments, the present application contemplates variants that possess some but not all effector functions, which make it a desirable candidate for applications in which the half-life of the binding molecule in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the binding molecule lacks FcγR binding (hence likely lacking ADCC activity), but retains FcRn binding ability. Non-limiting examples of in vitro assays to assess ADCC activity of a molecule of interest are described in U.S. Pat. No. 5,500,362 (see, e.g., Hellstrom, I. et al. Proc. Nat'l Acad. Sci. USA 83:7059-7063 (1986)) and Hellstrom, I et al., Proc. Nat'l Acad. Sci. USA 82:1499-1502 (1985); U.S. Pat. No. 5,821,337 (see Bruggemann, M. et al., J. Exp. Med. 166:1351-1361 (1987)). Alternatively, non-radioactive assay methods may be employed (see, for example, ACTI™ non-radioactive cytotoxicity assay for flow cytometry (CellTechnology, Inc. Mountain View, CA; and CytoTox 96® non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half-life determinations can also be performed using methods known in the art (see, e.g., Petkova, S. B. et al., Int'l. Immunol. 18 (12): 1759-1769 (2006)).

Binding molecules with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain variants with improved or diminished binding to FcRs are described. (See, e.g., U.S. Pat. No. 6,737,056; WO 2004/056312, and Shields et al., J. Biol. Chem. 9 (2): 6591-6604 (2001).)

In some embodiments, a variant comprises an Fc region with one or more amino acid substitutions which improve ADCC, e.g., substitutions at positions 298, 333, and/or 334 of the Fc region (EU numbering of residues). In some embodiments, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164:4178-4184 (2000).

Binding molecules with increased half-lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those molecules comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826). See also Duncan & Winter, Nature 322:738-40 (1988); U.S. Pat. Nos. 5,648,260; 5,624,821; and WO 94/29351 concerning other examples of Fc region variants.

In some embodiments, it may be desirable to create cysteine engineered antibodies, in which one or more residues of an antibody are substituted with cysteine residues. In some embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein.

Other known covalent modifications of antibodies are included within the scope of the present disclosure. Covalent modifications include reacting targeted amino acid residues of an antibody with an organic derivatizing agent that is capable of reacting with selected side chains or the N- or C-terminal residues of the antibody. Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the α-amino groups of lysine, arginine, and histidine side chains (see, e.g., Creighton, Proteins: Structure and Molecular Properties 79-86 (1983)), acetylation of the N-terminal amine, and amidation of any C-terminal carboxyl group.

The antibody that binds to LILRB1 and/or LILRB2 of the present disclosure (e.g. the anti-LILRB1 antibodies, the anti-LILRB2 antibodies, the multispecific antibodies that bind to both LILRB1 and LILRB2) may also be modified to form chimeric molecules comprising the antibody fused or conjugated to another, heterologous polypeptide or amino acid sequence or a small molecule compound, for example, an immune activator (such as a cytokine), an epitope tag (see, e.g., Terpe, Appl. Microbiol. Biotechnol. 60:523-33 (2003)) or the Fc region of an IgG molecule (see, e.g., Aruffo, Antibody Fusion Proteins 221-42 (Chamow and Ashkenazi eds., 1999)).

Also provided herein are fusion proteins comprising the antibody that binds to LILRB1 and/or LILRB2 of the disclosure (e.g. the anti-LILRB1 antibodies, the anti-LILRB2 antibodies, the multispecific antibodies that bind to both LILRB1 and LILRB2) and a heterologous polypeptide. In some embodiments, the heterologous polypeptide to which the antibody is genetically fused or chemically conjugated is useful for targeting the antibody to cells having cell surface-expressed LILRB1 and/or LILRB2. Genetically fused or chemically conjugated antibodies are described in more detail in sections below.

### In Vitro Affinity Maturation

In some embodiments, antibody variants having an improved property such as affinity, stability, or expression level as compared to a parent antibody may be prepared by in vitro affinity maturation. Like the natural prototype, in vitro affinity maturation is based on the principles of mutation and selection. Libraries of antibodies are displayed on the surface of an organism (e.g., phage, bacteria, yeast, or mammalian cell) or in association (e.g., covalently or non-covalently) with their encoding mRNA or DNA. Affinity selection of the displayed antibodies allows isolation of organisms or complexes carrying the genetic information encoding the antibodies. Two or three rounds of mutation and selection using display methods such as phage display usually results in antibody fragments with affinities in the low nanomolar range. Affinity matured antibodies can have nanomolar or even picomolar affinities for the target antigen.

Phage display is a widespread method for display and selection of antibodies. The antibodies are displayed on the surface of Fd or M13 bacteriophages as fusions to the bacteriophage coat protein. Selection involves exposure to antigen to allow phage-displayed antibodies to bind their targets, a process referred to as “panning.” Phage bound to antigen are recovered and used to infect bacteria to produce phage for further rounds of selection. For review, see, for example, Hoogenboom, Methods. Mol. Biol. 178:1-37 (2002); and Bradbury and Marks, J. Immunol. Methods 290:29-49 (2004).

In a yeast display system (see, e.g., Boder et al., Nat. Biotech. 15:553-57 (1997); and Chao et al., Nat. Protocols 1:755-68 (2006)), the antibody may be fused to the adhesion subunit of the yeast agglutinin protein Aga2p, which attaches to the yeast cell wall through disulfide bonds to Aga1p. Display of a protein via Aga2p projects the protein away from the cell surface, minimizing potential interactions with other molecules on the yeast cell wall. Magnetic separation and flow cytometry are used to screen the library to select for antibodies with improved affinity or stability. Binding to a soluble antigen of interest is determined by labeling yeast with biotinylated antigen and a secondary reagent such as streptavidin conjugated to a fluorophore. Variations in surface expression of the antibody can be measured through immunofluorescence labeling of either the hemagglutinin or c-Myc epitope tag flanking the single-chain antibody (e.g., scFv). Expression has been shown to correlate with the stability of the displayed protein, and thus antibodies can be selected for improved stability as well as affinity (see, e.g., Shusta et al., J. Mol. Biol. 292:949-56 (1999)). An additional advantage of yeast display is that displayed proteins are folded in the endoplasmic reticulum of the eukaryotic yeast cells, taking advantage of endoplasmic reticulum chaperones and quality-control machinery. Once maturation is complete, antibody affinity can be conveniently “titrated” while displayed on the surface of the yeast, eliminating the need for expression and purification of each clone. A theoretical limitation of yeast surface display is the potentially smaller functional library size than that of other display methods; however, a recent approach uses the yeast cells' mating system to create combinatorial diversity estimated to be 1014 in size (see, e.g., U.S. Pat. Publ. No. 2003/0186374; and Blaise et al., Gene 342:211-18 (2004)).

In ribosome display, antibody-ribosome-mRNA (ARM) complexes are generated for selection in a cell-free system. The DNA library coding for a particular library of antibodies is genetically fused to a spacer sequence lacking a stop codon. This spacer sequence, when translated, is still attached to the peptidyl tRNA and occupies the ribosomal tunnel, and thus allows the protein of interest to protrude out of the ribosome and fold. The resulting complex of mRNA, ribosome, and protein can bind to surface-bound ligand, allowing simultaneous isolation of the antibody and its encoding mRNA through affinity capture with the ligand. The ribosome-bound mRNA is then reverse transcribed back into cDNA, which can then undergo mutagenesis and be used in the next round of selection (see, e.g., Fukuda et al., Nucleic Acids Res. 34: e127 (2006)). In mRNA display, a covalent bond between antibody and mRNA is established using puromycin as an adaptor molecule (Wilson et al., Proc. Natl. Acad. Sci. USA 98:3750-55 (2001)).

As these methods are performed entirely in vitro, they provide two main advantages over other selection technologies. First, the diversity of the library is not limited by the transformation efficiency of bacterial cells, but only by the number of ribosomes and different mRNA molecules present in the test tube. Second, random mutations can be introduced easily after each selection round, for example, by non-proofreading polymerases, as no library must be transformed after any diversification step. In some embodiments, mammalian display systems may be used.

Diversity may also be introduced into the CDRs of the antibody libraries in a targeted manner or via random introduction. The former approach includes sequentially targeting all the CDRs of an antibody via a high or low level of mutagenesis or targeting isolated hot spots of somatic hypermutations (see, e.g., Ho et al., J. Biol. Chem. 280:607-17 (2005)) or residues suspected of affecting affinity on experimental basis or structural reasons. Diversity may also be introduced by replacement of regions that are naturally diverse via DNA shuffling or similar techniques (see, e.g., Lu et al., J. Biol. Chem. 278:43496-507 (2003); U.S. Pat. Nos. 5,565,332 and 6,989,250). Alternative techniques target hypervariable loops extending into framework-region residues (see, e.g., Bond et al., J. Mol. Biol. 348:699-709 (2005)) employ loop deletions and insertions in CDRs or use hybridization-based diversification (see, e.g., U.S. Pat. Publ. No. 2004/0005709). Additional methods of generating diversity in CDRs are disclosed, for example, in U.S. Pat. No. 7,985,840. Further methods that can be used to generate antibody libraries and/or antibody affinity maturation are disclosed, e.g., in U.S. Pat. Nos. 8,685,897 and 8,603,930, and U.S. Publ. Nos. 2014/0170705, 2014/0094392, 2012/0028301, 2011/0183855, and 2009/0075378, each of which is incorporated herein by reference.

Screening of the libraries can be accomplished by various techniques known in the art. For example, antibodies can be immobilized onto solid supports, columns, pins, or cellulose/poly (vinylidene fluoride) membranes/other filters, expressed on host cells affixed to adsorption plates or used in cell sorting, or conjugated to biotin for capture with streptavidin-coated beads or used in any other method for panning display libraries.

For reviews of in vitro affinity maturation methods, see, e.g., Hoogenboom, Nature Biotechnology 23:1105-16 (2005); Quiroz and Sinclair, Revista Ingeneria Biomedia 4:39-51 (2010); and references therein.

An antibody internalization assay may be used to determine receptor-mediated endocytosis when binding to an antibody. In some embodiments, the efficacy of certain antibody-based therapeutics depends on antibody internalization process. In some embodiments, an antibody internalization assay examines the rate and extent of antibody internalization in order to evaluate the antibody's ability of delivering treatments to sites or cells of interest. A non-limiting exemplary assay is briefly described below. Target cells of interest are seeded at an appropriate seeding density (e.g., in a 96-well U-bottom plate), and a tested antibody is labelled with a signal reporting reagent, for example, fluorescent compounds, Horseradish peroxidase (HRP) reagent, radiolabeled compounds, or biotin. Then the tested antibody and the target cells are incubated at an appropriate molar ratio. Following the incubation, unbound antibodies are removed by wash. The cells can be left on ice or incubated at 37° C. for a period of time to facilitate internalization. The cells may then be incubated for a period of time in the presence of a stop reagent to inhibit internalization. Subsequently, the cells are washed and incubated with the signal developing reagent. The final signal can be studied using plate reader or imaging instrument and an analytical software. For example, mean fluorescence intensity (MFI) of the cells can be measured using a flow cytometer, and MFI reduction can represent antibody internalization, antibody dissociation or a combination of both. Cell imaging can be scanned and acquired to analyze the signal intensity, size and shape. Alternatively, the cells are lysed, releasing internalized antibody. This antibody is then captured in a microtiter well plate coated with specific antigen against which the antibody was raised. Bound antibody in the well is detected using an alkaline phosphatase or HRP-conjugated secondary antibody and a chromogenic substrate. Alternative detectable labels for the antibody and means for detecting internalized labeled antibody will be obvious to those skilled in the art upon this disclosure. Any methods known in the art to determine antibody internalization can be used in the present disclosure.

### 5.2.7. Other Binding Molecules Comprising the Antibodies

In another aspect, provided herein is a binding molecule comprising an anti-LILRB1 and/or LILRB2 antibody provided herein (e.g. the anti-LILRB1 antibody, the anti-LILRB2 antibody, or the multispecific antibody that bind to both LILRB1 and LILRB2). In some embodiments, an antibody against LILRB1 and/or LILRB2 provided herein is part of other binding molecules. Exemplary binding molecules of the present disclosure are described herein.

### Fusion Protein

In various embodiments, the antibody provided herein can be genetically fused or chemically conjugated to another agent, for example, protein-based entities. The antibody may be chemically-conjugated to the agent, or otherwise non-covalently conjugated to the agent. The agent can be a peptide or antibody (or a fragment thereof).

Thus, in some embodiments, provided herein are antibodies that are recombinantly fused or chemically conjugated (covalent or non-covalent conjugations) to a heterologous protein or polypeptide (or fragment thereof, for example, to a polypeptide of about 10, about 20, about 30, about 40, about 50, about 60, about 70, about 80, about 90, about 100, about 150, about 200, about 250, about 300, about 350, about 400, about 450 or about 500 amino acids, or over 500 amino acids) to generate fusion proteins, as well as uses thereof. In particular, provided herein are fusion proteins comprising an antigen binding fragment of the antibody provided herein (e.g., CDR1, CDR2, and/or CDR3) and a heterologous protein, polypeptide, or peptide.

Moreover, antibodies provided herein can be fused to marker or “tag” sequences, such as a peptide, to facilitate purification. In specific embodiments, the marker or tag amino acid sequence is a hexa-histidine peptide, hemagglutinin (“HA”) tag, and “FLAG” tag.

Methods for fusing or conjugating moieties (including polypeptides) to antibodies are known (see, e.g., Arnon et al., Monoclonal Antibodies for Immunotargeting of Drugs in Cancer Therapy, in Monoclonal Antibodies and Cancer Therapy 243-56 (Reisfeld et al. eds., 1985); Hellstrom et al., Antibodies for Drug Delivery, in Controlled Drug Delivery 623-53 (Robinson et al. eds., 2d ed. 1987); Thorpe, Antibody Carriers of Cytotoxic Agents in Cancer Therapy: A Review, in Monoclonal Antibodies: Biological and Clinical Applications 475-506 (Pinchera et al. eds., 1985); Analysis, Results, and Future Prospective of the Therapeutic Use of Radiolabeled Antibody in Cancer Therapy, in Monoclonal Antibodies for Cancer Detection and Therapy 303-16 (Baldwin et al. eds., 1985); Thorpe et al., Immunol. Rev. 62:119-58 (1982); U.S. Pat. Nos. 5,336,603; 5,622,929; 5,359,046; 5,349,053; 5,447,851; 5,723,125; 5,783,181; 5,908,626; 5,844,095; and 5,112,946; EP 307,434; EP 367,166; EP 394,827; PCT publications WO 91/06570, WO 96/04388, WO 96/22024, WO 97/34631, and WO 99/04813; Ashkenazi et al., Proc. Natl. Acad. Sci. USA, 88:10535-39 (1991); Traunecker et al., Nature, 331:84-86 (1988); Zheng et al., J. Immunol. 154:5590-600 (1995); and Vil et al., Proc. Natl. Acad. Sci. USA 89:11337-41 (1992)).

Fusion proteins may be generated, for example, through the techniques of gene-shuffling, motif-shuffling, exon-shuffling, and/or codon-shuffling (collectively referred to as “DNA shuffling”). DNA shuffling may be employed to alter the activities of the antibodies as provided herein, including, for example, antibodies with higher affinities and lower dissociation rates (see, e.g., U.S. Pat. Nos. 5,605,793; 5,811,238; 5,830,721; 5,834,252; and U.S. Pat. No. 5,837,458; Patten et al., Curr. Opinion Biotechnol. 8:724-33 (1997); Harayama, Trends Biotechnol. 16 (2): 76-82 (1998); Hansson et al., J. Mol. Biol. 287:265-76 (1999); and Lorenzo and Blasco, Biotechniques 24 (2): 308-13 (1998)). Antibodies, or the encoded antibodies, may be altered by being subjected to random mutagenesis by error-prone PCR, random nucleotide insertion, or other methods prior to recombination. A polynucleotide encoding an antibody provided herein may be recombined with one or more components, motifs, sections, parts, domains, fragments, etc. of one or more heterologous molecules.

In some embodiments, an antibody provided herein is conjugated to a second antibody to form an antibody heteroconjugate.

In various embodiments, the antibody is genetically fused to the agent. Genetic fusion may be accomplished by placing a linker (e.g., a polypeptide) between the antibody and the agent. The linker may be a flexible linker.

In various embodiments, the antibody is genetically conjugated to a therapeutic molecule, with a hinge region linking the antibody to the therapeutic molecule.

Also provided herein are methods for making the various fusion proteins provided herein. The various methods described in Section 5.4 may also be utilized to make the fusion proteins provided herein.

In a specific embodiment, the fusion protein provided herein is recombinantly expressed. Recombinant expression of a fusion protein provided herein may require construction of an expression vector containing a polynucleotide that encodes the protein or a fragment thereof. Once a polynucleotide encoding a protein provided herein or a fragment thereof has been obtained, the vector for the production of the molecule may be produced by recombinant DNA technology using techniques well-known in the art. Thus, methods for preparing a protein by expressing a polynucleotide containing an encoding nucleotide sequence are described herein. Methods which are well known to those skilled in the art can be used to construct expression vectors containing coding sequences and appropriate transcriptional and translational control signals. These methods include, for example, in vitro recombinant DNA techniques, synthetic techniques, and in vivo genetic recombination. Also provided are replicable vectors comprising a nucleotide sequence encoding a fusion protein provided herein, or a fragment thereof, or a CDR, operably linked to a promoter.

The expression vector can be transferred to a host cell by conventional techniques and the transfected cells are then cultured by conventional techniques to produce a fusion protein provided herein. Thus, also provided herein are host cells containing a polynucleotide encoding a fusion protein provided herein or fragments thereof operably linked to a heterologous promoter.

A variety of host-expression vector systems may be utilized to express the fusion protein provided herein. Such host-expression systems represent vehicles by which the coding sequences of interest may be produced and subsequently purified, but also represent cells which may, when transformed or transfected with the appropriate nucleotide coding sequences, express a fusion protein provided herein in situ. These include but are not limited to microorganisms such as bacteria (e.g., E. coli and B. subtilis) transformed with recombinant bacteriophage DNA, plasmid DNA or cosmid DNA expression vectors containing coding sequences; yeast (e.g., Saccharomyces Pichia) transformed with recombinant yeast expression vectors containing coding sequences; insect cell systems infected with recombinant virus expression vectors (e.g., baculovirus) containing coding sequences; plant cell systems infected with recombinant virus expression vectors (e.g., cauliflower mosaic virus, CaMV, tobacco mosaic virus, TMV) or transformed with recombinant plasmid expression vectors (e.g., Ti plasmid) containing coding sequences; or mammalian cell systems (e.g., COS, CHO, BHK, 293, NS0, and 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Bacterial cells such as Escherichia coli, or, eukaryotic cells, especially for the expression of whole recombinant antibody molecule, can be used for the expression of a recombinant fusion protein. For example, mammalian cells such as Chinese hamster ovary cells (CHO), in conjunction with a vector such as the major intermediate early gene promoter element from human cytomegalovirus is an effective expression system for antibodies or variants thereof. In a specific embodiment, the expression of nucleotide sequences encoding the fusion proteins provided herein is regulated by a constitutive promoter, inducible promoter or tissue specific promoter.

In bacterial systems, a number of expression vectors may be advantageously selected depending upon the use intended for the fusion protein being expressed. For example, when a large quantity of such a fusion protein is to be produced, for the generation of pharmaceutical compositions of a fusion protein, vectors which direct the expression of high levels of fusion protein products that are readily purified may be desirable. Such vectors include, but are not limited to, the E. coli expression vector pUR278 (Ruther et al., EMBO 12:1791 (1983)), in which the coding sequence may be ligated individually into the vector in frame with the lac Z coding region so that a fusion protein is produced; pIN vectors (Inouye & Inouye, Nucleic Acids Res. 13:3101-3109 (1985); Van Heeke & Schuster, J. Biol. Chem. 24:5503-5509 (1989)); and the like. pGEX vectors may also be used to express foreign polypeptides as fusion proteins with glutathione 5-transferase (GST). In general, such fusion proteins are soluble and can easily be purified from lysed cells by adsorption and binding to matrix glutathione agarose beads followed by elution in the presence of free glutathione. The pGEX vectors are designed to include thrombin or factor Xa protease cleavage sites so that the cloned target gene product can be released from the GST moiety.

In mammalian host cells, a number of viral-based expression systems may be utilized. In cases where an adenovirus is used as an expression vector, the coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the fusion protein in infected hosts (e.g., see Logan & Shenk, Proc. Natl. Acad. Sci. USA 8 1:355-359 (1984)). Specific initiation signals may also be required for efficient translation of inserted coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see, e.g., Bittner et al., Methods in Enzymol. 153:51-544 (1987)).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERY, BHK, Hela, COS, MDCK, 293, 3T3, W138, BT483, Hs578T, HTB2, BT20 and T47D, NS0 (a murine myeloma cell line that does not endogenously produce any immunoglobulin chains), CRL7030 and HsS78Bst cells.

For long-term, high-yield production of recombinant proteins, stable expression can be utilized. For example, cell lines which stably express the fusion proteins may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the fusion protein. Such engineered cell lines may be particularly useful in screening and evaluation of compositions that interact directly or indirectly with the binding molecule.

A number of selection systems may be used, including but not limited to, the herpes simplex virus thymidine kinase (Wigler et al., Cell 11:223 (1977)), hypoxanthineguanine phosphoribosyltransferase (Szybalska & Szybalski, Proc. Natl. Acad. Sci. USA 48:202 (1992)), and adenine phosphoribosyltransferase (Lowy et al., Cell 22:8-17 (1980)) genes can be employed in tk-, hgprt- or aprt-cells, respectively. Also, antimetabolite resistance can be used as the basis of selection for the following genes: dhfr, which confers resistance to methotrexate (Wigler et al., Natl. Acad. Sci. USA 77:357 (1980); O'Hare et al., Proc. Natl. Acad. Sci. USA 78:1527 (1981)); gpt, which confers resistance to mycophenolic acid (Mulligan & Berg, Proc. Natl. Acad. Sci. USA 78:2072 (1981)); neo, which confers resistance to the aminoglycoside G-418 (Wu and Wu, Biotherapy 3:87-95 (1991); Tolstoshev, Ann. Rev. Pharmacol. Toxicol. 32:573-596 (1993); Mulligan, Science 260:926-932 (1993); and Morgan and Anderson, Ann. Rev. Biochem. 62:191-217 (1993); May, TIB TECH 11 (5): 155-2 15 (1993)); and hygro, which confers resistance to hygromycin (Santerre et al., Gene 30:147 (1984)). Methods commonly known in the art of recombinant DNA technology may be routinely applied to select the desired recombinant clone, and such methods are described, for example, in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds.), Current Protocols in Human Genetics, John Wiley & Sons, NY (1994); Colberre-Garapin et al., J. Mol. Biol. 150:1 (1981), which are incorporated by reference herein in their entireties.

The expression level of a fusion protein can be increased by vector amplification (for a review, see Bebbington and Hentschel, The use of vectors based on gene amplification for the expression of cloned genes in mammalian cells in DNA cloning, Vol. 3 (Academic Press, New York, 1987)). When a marker in the vector system expressing a fusion protein is amplifiable, increase in the level of inhibitor present in culture of host cell will increase the number of copies of the marker gene. Since the amplified region is associated with the fusion protein gene, production of the fusion protein will also increase (Crouse et al., Mol. Cell. Biol. 3:257 (1983)).

The host cell may be co-transfected with multiple expression vectors provided herein. The vectors may contain identical selectable markers which enable equal expression of respective encoding polypeptides. Alternatively, a single vector may be used which encodes, and is capable of expressing multiple polypeptides. The coding sequences may comprise cDNA or genomic DNA.

Once a fusion protein provided herein has been produced by recombinant expression, it may be purified by any method known in the art for purification of a polypeptide (e.g., an immunoglobulin molecule), for example, by chromatography (e.g., ion exchange, affinity, particularly by affinity for the specific antigen after Protein A, sizing column chromatography, and Kappa select affinity chromatography), centrifugation, differential solubility, or by any other standard technique for the purification of proteins. Further, the fusion protein molecules provided herein can be fused to heterologous polypeptide sequences described herein or otherwise known in the art to facilitate purification.

### Immunoconjugates

In some embodiments, the present disclosure also provides immunoconjugates comprising any of the anti-LILRB1 and/or LILRB2 antibodies described herein (e.g. the anti-LILRB1 antibodies, the anti-LILRB2 antibodies, the multispecific antibodies that bind to both LILRB1 and LILRB2) conjugated to one or more cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes.

In some embodiments, an immunoconjugate is an antibody-drug conjugate (ADC) in which an antibody is conjugated to one or more drugs, including but not limited to a maytansinoid (see U.S. Pat. Nos. 5,208,020, 5,416,064 and European Patent EP 0 425 235 B1); an auristatin such as monomethylauristatin drug moieties DE and DF (MMAE and MMAF) (see U.S. Pat. Nos. 5,635,483 and 5,780,588, and 7,498,298); a dolastatin; a calicheamicin or derivative thereof (see U.S. Pat. Nos. 5,712,374, 5,714,586, 5,739,116, 5,767,285, 5,770,701, 5,770,710, 5,773,001, and 5,877,296; Hinman et al., Cancer Res. 53:3336-3342 (1993); and Lode et al., Cancer Res. 58:2925-2928 (1998)); an anthracycline such as daunomycin or doxorubicin (see Kratz et al., Current Med. Chem. 13:477-523 (2006); Jeffrey et al., Bioorganic & Med. Chem. Letters 16:358-362 (2006); Torgov et al., Bioconj. Chem. 16:717-721 (2005); Nagy et al., Proc. Natl. Acad. Sci. USA 97:829-834 (2000); Dubowchik et al., Bioorg. & Med. Chem. Letters 12:1529-1532 (2002); King et al., J. Med. Chem. 45:4336-4343 (2002); and U.S. Pat. No. 6,630,579); methotrexate; vindesine; a taxane such as docetaxel, paclitaxel, larotaxel, tesetaxel, and ortataxel; a trichothecene; and CC1065.

In some embodiments, an immunoconjugate comprises an antibody as described herein conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes.

In some embodiments, an immunoconjugate comprises an antibody as described herein conjugated to a radioactive atom to form a radioconjugate. A variety of radioactive isotopes are available for the production of radioconjugates. Examples include At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu. When the radioconjugate is used for detection, it may comprise a radioactive atom for scintigraphic studies, for example tc99m or I123, or a spin label for nuclear magnetic resonance (NMR) imaging (also known as magnetic resonance imaging, mri), such as iodine-123 again, iodine-131, indium-111, fluorine-19, carbon-13, nitrogen-15, oxygen-17, gadolinium, manganese or iron.

Conjugates of an antibody and cytotoxic agent may be made using a variety of bifunctional protein coupling agents such as N-succinimidyl-3-(2-pyridyldithio) propionate (SPDP), succinimidyl-4-(N-maleimidomethyl)cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis(p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

The linker may be a “cleavable linker” facilitating release of the conjugated agent in the cell, but non-cleavable linkers are also contemplated herein. Linkers for use in the conjugates of the present disclosure include, without limitation, acid labile linkers (e.g., hydrazone linkers), disulfide-containing linkers, peptidase-sensitive linkers (e.g., peptide linkers comprising amino acids, for example, valine and/or citrulline such as citrulline-valine or phenylalanine-lysine), photolabile linkers, dimethyl linkers, thioether linkers, or hydrophilic linkers designed to evade multidrug transporter-mediated resistance.

The immunuoconjugates or ADCs herein contemplate, but are not limited to such conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, IL., U.S.A).

In other embodiments, antibodies provided herein are conjugated or recombinantly fused, e.g., to a diagnostic molecule. Such diagnosis and detection can be accomplished, for example, by coupling the antibody to detectable substances including, but not limited to, various enzymes, such as, but not limited to, horseradish peroxidase, alkaline phosphatase, beta-galactosidase, or acetylcholinesterase; prosthetic groups, such as, but not limited to, streptavidin/biotin or avidin/biotin; fluorescent materials, such as, but not limited to, umbelliferone, fluorescein, fluorescein isothiocynate, rhodamine, dichlorotriazinylamine fluorescein, dansyl chloride, or phycoerythrin; luminescent materials, such as, but not limited to, luminol; bioluminescent materials, such as, but not limited to, luciferase, luciferin, or aequorin; chemiluminescent material, such as, 225Acγ-emitting, Auger-emitting, β-emitting, an alpha-emitting or positron-emitting radioactive isotope.

### 5.3. Polynucleotides

Additionally provided are a nucleic acid encoding a binding agent (e.g., an anti-LILRB1 antibody, an anti-LILRB2 antibody, or a multispecific antibody that binds to both LILRB1 and LILRB2) or a fusion polypeptide as disclosed herein, a nucleic acid complementary thereto; a vector comprising a nucleic acid as disclosed herein; and a cell comprising any one or more of: a binding agent as disclosed herein, a nucleic acid as disclosed herein, or a vector as disclosed herein. In some embodiments, the cell expresses the binding agent provided herein. In some embodiments, the cell replicates the nucleic acid or the vector. In some embodiments, provided are the materials for generating binding agents and fragments thereof. For example, an isolated cell may produce a binding agent (e.g., an anti-LILRB1 antibody, an anti-LILRB2 antibody, or a multispecific antibody that binds to both LILRB1 and LILRB2). In this regard, a cell (e.g., an isolated cell) may produce an antibody or fragment thereof comprising a VH and a VL as disclosed herein. In some embodiments, polynucleotides described herein may comprise one or more nucleic acid sequences encoding a binding agent (e.g., antibody or antibody fragment). In some embodiments, the polynucleotide is an isolated and/or recombinant polynucleotide. In various aspects, the isolated polynucleotide comprises a nucleotide sequence that encodes a VH and/or a VL, wherein the VH and the VL comprise complementarity determining regions (CDRs) identical to CDRs as disclosed herein.

As used herein, the term “complementary” refers to specific binding between polynucleotides based on the sequences of the polynucleotides. As used herein, a first polynucleotide and a second polynucleotide are complementary if they bind to each other in a hybridization assay under stringent conditions, e.g., if they produce a given or detectable level of signal in a hybridization assay. Portions of polynucleotides are complementary to each other if they follow conventional base-pairing rules, e.g., A pairs with T (or U) and G pairs with C, although small regions (e.g., fewer than about 3 bases) of mismatch, insertion, or deleted sequence may be present. The term “stringent assay conditions” refers to conditions that are compatible to produce binding pairs of nucleic acids, e.g., probes and target mRNAs, of sufficient complementarity to provide for the desired level of specificity in the assay while being generally incompatible to the formation of binding pairs between binding members of insufficient complementarity to provide for the desired specificity. The term “stringent assay conditions” generally refers to the combination of hybridization and wash conditions.

In some embodiments, one or more vectors (e.g., expression vectors) may comprise one or more polynucleotides for expression of the one or more polynucleotides in a suitable host cell. Such vectors are useful, for example, for amplifying the polynucleotides in host cells to create useful quantities thereof, and for expressing binding agents, such as antibodies or antibody fragments, using recombinant techniques.

In some embodiments, one or more vectors are expression vectors wherein one or more polynucleotides are operatively linked to one or more polynucleotides comprising expression control sequences. Autonomously replicating recombinant expression constructs such as plasmid and viral DNA vectors incorporating one or more polynucleotides encoding antibody sequences that bind LILRB1 and/or LILRB2 are specifically contemplated. Expression control DNA sequences include promoters, enhancers, and operators, and are generally selected based on the expression systems in which the expression construct is to be utilized. Promoter and enhancer sequences are generally selected for the ability to increase gene expression, while operator sequences are generally selected for the ability to regulate gene expression. Expression constructs may also include sequences encoding one or more selectable markers that permit identification of host cells bearing the construct. Expression constructs may also include sequences that facilitate, and preferably promote, homologous recombination in a host cell. In some embodiments, expression constructs of the can also include sequences necessary for replication in a host cell.

Exemplary expression control sequences include promoter/enhancer sequences, e.g., cytomegalovirus promoter/enhancer (Lehner et al., J. Clin. Microbiol., 29:2494-2502, 1991; Boshart et al., Cell, 41:521-530, 1985); Rous sarcoma virus promoter (Davis et al., Hum. Gene Ther., 4:151, 1993); Tie promoter (Korhonen et al., Blood, 86 (5): 1828-1835, 1995); simian virus 40 promoter; DRA (downregulated in adenoma; Alrefai et al., Am. J. Physiol. Gastrointest. Liver Physiol., 293: G923-G934, 2007); MCT1 (monocarboxylate transporter 1; Cuff et al., Am. J. Physiol. Gastrointet. Liver Physiol., G977-G979. 2005); and Math1 (mouse atonal homolog 1; Shroyer et al., Gastroenterology, 132:2477-2478, 2007), for expression in mammalian cells, the promoter being operatively linked upstream (e.g., 5′) of a polypeptide coding sequence. In another variation, the promoter is an epithelial-specific promoter or endothelial-specific promoter. Polynucleotides may also optionally include a suitable polyadenylation sequence (e.g., the SV40 or human growth hormone gene polyadenylation sequence) operably linked downstream (e.g., 3′) of the polypeptide coding sequence.

If desired, the one or more polynucleotides also optionally comprise nucleotide sequences encoding secretory signal peptides fused in frame with the polypeptide sequences. The secretory signal peptides direct secretion of the antibody polypeptides by the cells that express the one or more polynucleotides, and are cleaved by the cell from the secreted polypeptides. The one or more polynucleotides may further optionally comprise sequences whose only intended function is to facilitate large scale production of the vector. One can manufacture and administer polynucleotides for gene therapy using procedures that have been described in the literature for a variety of transgenes. See, e.g., Isner et al., Circulation, 91:2687-2692, 1995; and Isner et al., Human Gene Therapy, 7:989-1011, 1996.

In some embodiments, polynucleotides may further comprise additional sequences to facilitate uptake by host cells and expression of the antibody or fragment thereof (and/or any other peptide). In some embodiments, a “naked” transgene encoding an antibody or fragment thereof described herein (e.g., a transgene without a viral, liposomal, or other vector to facilitate transfection) is employed.

The polynucleotides of the disclosure can be in the form of RNA or in the form of DNA. DNA includes cDNA, genomic DNA, and synthetic DNA; and can be double-stranded or single-stranded, and if single stranded can be the coding strand or non-coding (anti-sense) strand. In some embodiments, the polynucleotide is in the form of cDNA. In some embodiments, the polynucleotide is a synthetic polynucleotide.

The present disclosure further relates to variants of the polynucleotides described herein, wherein the variant encodes, for example, fragments, analogs, and/or derivatives of the binding molecules of the disclosure. In certain embodiments, the present disclosure provides a polynucleotide comprising a polynucleotide having a nucleotide sequence at least about 75% identical, at least about 80% identical, at least about 85% identical, at least about 90% identical, at least about 95% identical, and in some embodiments, at least about 96%, 97%, 98% or 99% identical to a polynucleotide encoding the binding molecule of the disclosure. As used herein, the phrase “a polynucleotide having a nucleotide sequence at least, for example, 95% “identical” to a reference nucleotide sequence is intended to mean that the nucleotide sequence of the polynucleotide is identical to the reference sequence except that the polynucleotide sequence can include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence can be deleted or substituted with another nucleotide, or a number of nucleotides up to 5% of the total nucleotides in the reference sequence can be inserted into the reference sequence. These mutations of the reference sequence can occur at the 5′ or 3′ terminal positions of the reference nucleotide sequence or anywhere between those terminal positions, interspersed either individually among nucleotides in the reference sequence or in one or more contiguous groups within the reference sequence.

The polynucleotide variants can contain alterations in the coding regions, non-coding regions, or both. In some embodiments, a polynucleotide variant contains alterations which produce silent substitutions, additions, or deletions, but does not alter the properties or activities of the encoded polypeptide. In some embodiments, a polynucleotide variant comprises silent substitutions that results in no change to the amino acid sequence of the polypeptide (due to the degeneracy of the genetic code). Polynucleotide variants can be produced for a variety of reasons, for example, to optimize codon expression for a particular host (i.e., change codons in the human mRNA to those preferred by a bacterial host such as E. coli). In some embodiments, a polynucleotide variant comprises at least one silent mutation in a non-coding or a coding region of the sequence.

In some embodiments, a polynucleotide variant is produced to modulate or alter expression (or expression levels) of the encoded polypeptide. In some embodiments, a polynucleotide variant is produced to increase expression of the encoded polypeptide. In some embodiments, a polynucleotide variant is produced to decrease expression of the encoded polypeptide. In some embodiments, a polynucleotide variant has increased expression of the encoded polypeptide as compared to a parental polynucleotide sequence. In some embodiments, a polynucleotide variant has decreased expression of the encoded polypeptide as compared to a parental polynucleotide sequence.

Any suitable vectors may be used to introduce one or more polynucleotides that encode an antibody or fragment thereof into the host. Exemplary vectors that have been described include replication deficient retroviral vectors, including but not limited to lentivirus vectors (Kim et al., J. Virol., 72 (1): 811-816, 1998; Kingsman & Johnson, Scrip Magazine, October 1998, pp. 43-46); parvoviral vectors, such as adeno-associated viral (AAV) vectors (U.S. Pat. Nos. 5,474,9351; 5,139,941; 5,622,856; 5,658,776; 5,773,289; 5,789,390; 5,834,441; 5,863,541; 5,851,521; 5,252,479; Gnatenko et al., J. Invest. Med., 45:87-98, 1997); adenoviral (AV) vectors (U.S. Pat. Nos. 5,792,453; 5,824,544; 5,707,618; 5,693,509; 5,670,488; 5,585,362; Quantin et al., Proc. Natl. Acad. Sci. USA, 89:2581-2584, 1992; Stratford Perricaudet et al., J. Clin. Invest., 90:626-630, 1992; and Rosenfeld et al., Cell, 68:143-155, 1992); an adenoviral adeno-associated viral chimeric (U.S. Pat. No. 5,856,152) or a vaccinia viral or a herpesviral vector (U.S. Pat. Nos. 5,879,934; 5,849,571; 5,830,727; 5,661,033; 5,328,688); Lipofectin mediated gene transfer (BRL); liposomal vectors (U.S. Pat. No. 5,631,237); and combinations thereof. Any of these expression vectors can be prepared using standard recombinant DNA techniques described in, e.g., Sambrook et al., Molecular Cloning, a Laboratory Manual, 2d edition, Cold Spring Harbor Press, Cold Spring Harbor, N.Y. (1989), and Ausubel et al., Current Protocols in Molecular Biology, Greene Publishing Associates and John Wiley & Sons, New York, N.Y. (1994). Optionally, viral vectors are rendered replication-deficient by, e.g., deleting or disrupting select genes required for viral replication.

Other non-viral delivery mechanisms contemplated include calcium phosphate precipitation (Graham and Van Der Eb, Virology, 52:456-467, 1973; Chen and Okayama, Mol. Cell Biol., 7:2745-2752, 1987; Rippe et al., Mol. Cell Biol., 10:689-695, 1990) DEAE-dextran (Gopal, Mol. Cell Biol., 5:1188-1190, 1985), electroporation (Tur-Kaspa et al., Mol. Cell Biol., 6:716-718, 1986; Potter et al., Proc. Nat. Acad. Sci. USA, 81:7161-7165, 1984), direct microinjection (Harland and Weintraub, J. Cell Biol., 101:1094-1099, 1985), DNA-loaded liposomes (Nicolau and Sene, Biochim. Biophys. Acta, 721:185-190, 1982; Fraley et al., Proc. Natl. Acad. Sci. USA, 76:3348-3352, 1979; Felgner, Sci Am., 276 (6): 102-6, 1997; Felgner, Hum Gene Ther., 7 (15): 1791-3, 1996), cell sonication (Fechheimer et al., Proc. Natl. Acad. Sci. USA, 84:8463-8467, 1987), gene bombardment using high velocity microprojectiles (Yang et al., Proc. Natl. Acad. Sci USA, 87:9568-9572, 1990), and receptor-mediated transfection (Wu and Wu, J. Biol. Chem., 262:4429-4432, 1987; Wu and Wu, Biochemistry, 27:887-892, 1988; Wu and Wu, Adv. Drug Delivery Rev., 12:159-167, 1993).

A vector (or the antibody or fragment thereof or a nucleic acid as discussed herein) may be entrapped in a liposome. See, e.g., Ghosh and Bachhawat, In: Liver diseases, targeted diagnosis and therapy using specific receptors and ligands, Wu G, Wu C ed., New York: Marcel Dekker, pp. 87-104 (1991); Radler et al., Science, 275 (5301): 810-814, (1997). Also contemplated are various commercial approaches involving “lipofection” technology. In some embodiments, the liposome may be complexed with a hemagglutinating virus (HVJ). This has been shown to facilitate fusion with the cell membrane and promote cell entry of liposome-encapsulated DNA (Kaneda et al., Science, 243:375-378, 1989). In some embodiments, the liposome is complexed or employed in conjunction with nuclear nonhistone chromosomal proteins (HMG-1) (Kato et al., J. Biol. Chem., 266:3361-3364, 1991). In some embodiments, the liposome is complexed or employed in conjunction with both HVJ and HMG-1. Such expression constructs have been successfully employed in transfer and expression of nucleic acid in vitro and in vivo. In some embodiments, a binding agent (e.g., an antibody) is included in the liposome to target the liposome to cells (such as an immune cell) expressing LILRB1 and/or LILRB2 on their surface.

A cell may comprise one or more polynucleotides or one or more vectors, e.g., the cell is transformed or transfected with one or more polynucleotides encoding a binding agent (e.g., an antibody) or the one or more vectors comprising the one or more polynucleotides. In some embodiments, cells express a binding agent (e.g., an antibody) containing one or more, including six CDRs having at least 75%, 80%, 85%, 90%, 95%, 96%, 97%, 98%, or 99% identity to the CDRs of Tables 5-6, and 14-15. In some embodiments, the cell expresses a binding agent (e.g., an antibody) containing the VH and the VL comprising CDRs identical to those of Tables 5-6, and 14-15. The cells may be prokaryotic cells, such as Escherichia coli (see, e.g., Pluckthun et al., Methods Enzymol., 178:497-515, 1989), or eukaryotic cells, such as an animal cell (e.g., a myeloma cell, Chinese Hamster Ovary (CHO) cell, or hybridoma cell), yeast (e.g., Saccharomyces cerevisiae), an insect cell, or a plant cell (e.g., a tobacco, corn, soybean, or rice cell). Use of mammalian host cells may provide for translational modifications (e.g., glycosylation, truncation, lipidation, and phosphorylation) that may be desirable to confer optimal biological activity on recombinant expression products. Similarly, polypeptides (e.g., binding agents (e.g., anti-LILRB1 antibodies, anti-LILRB2 antibodies, or multispecific antibodies that bind to both LILRB1 and LILRB2) may be glycosylated or non-glycosylated and/or have been covalently modified to include one or more water soluble polymer attachments such as polyethylene glycol, polyoxyethylene glycol, or polypropylene glycol.

Methods for introducing DNA or RNA into host cells are well known and include transformation, transfection, electroporation, nuclear injection, or fusion with carriers such as liposomes, micelles, ghost cells, and protoplasts. Such host cells are useful for amplifying polynucleotides and also for expressing polypeptides encoded by the polynucleotides. In this regard, a process for the production of a binding agent (e.g., an antibody) may comprise culturing a host cell and isolating the binding agent. Transferring a naked DNA expression construct into cells can be accomplished using particle bombardment, which depends on the ability to accelerate DNA coated microprojectiles to a high velocity allowing them to pierce cell membranes and enter cells without killing them (Klein et al., Nature, 327:70-73, 1987). Several devices for accelerating small particles have been developed. One such device relies on a high voltage discharge to generate an electrical current, which in turn provides the motive force (Yang et al., Proc. Natl. Acad. Sci USA, 87:9568-9572, 1990). The microprojectiles used have consisted of biologically inert substances such as tungsten or gold beads. A host cell may be isolated and/or purified. A host cell also may be a cell transformed in vivo to cause transient or permanent expression of the polypeptide in vivo. A host cell may also be an isolated cell transformed ex vivo and introduced post-transformation, e.g., to produce the polypeptide in vivo for therapeutic purposes. The definition of host cell explicitly excludes a transgenic human being.

### 5.4. Preparation of Antibodies and Method of Making

Methods of preparing antibodies have been described. See, e.g., Els Pardon et al, Nature Protocol, 9 (3): 674 (2014). Antibodies (such as scFv fragments) may be obtained using methods known in the art such as by immunizing a Camelid species (such as camel or llama) and obtaining hybridomas therefrom, or by cloning a library of antibodies using molecular biology techniques known in the art and subsequent selection by ELISA with individual clones of unselected libraries or by using phage display.

Antibodies provided herein may be produced by culturing cells transformed or transfected with a vector containing an antibody-encoding nucleic acids. Polynucleotide sequences encoding polypeptide components of the antibody of the present disclosure can be obtained using standard recombinant techniques. Desired polynucleotide sequences may be isolated and sequenced from antibody producing cells such as hybridomas cells or B cells. Alternatively, polynucleotides can be synthesized using nucleotide synthesizer or PCR techniques. Once obtained, sequences encoding the polypeptides are inserted into a recombinant vector capable of replicating and expressing heterologous polynucleotides in host cells. Many vectors that are available and known in the art can be used for the purpose of the present disclosure. Selection of an appropriate vector will depend mainly on the size of the nucleic acids to be inserted into the vector and the particular host cell to be transformed with the vector. Host cells suitable for expressing antibodies of the present disclosure include prokaryotes such as Archaebacteria and Eubacteria, including Gram-negative or Gram-positive organisms, eukaryotic microbes such as filamentous fungi or yeast, invertebrate cells such as insect or plant cells, and vertebrate cells such as mammalian host cell lines. Host cells are transformed with the above-described expression vectors and cultured in conventional nutrient media modified as appropriate for inducing promoters, selecting transformants, or amplifying the genes encoding the desired sequences. Antibodies produced by the host cells are purified using standard protein purification methods as known in the art.

Methods for antibody production including vector construction, expression, and purification are further described in Plückthun et al., Antibody Engineering: Producing antibodies in Escherichia coli: From PCR to fermentation 203-52 (McCafferty et al. eds., 1996); Kwong and Rader, E. coli Expression and Purification of Fab Antibody Fragments, in Current Protocols in Protein Science (2009); Tachibana and Takekoshi, Production of Antibody Fab Fragments in Escherichia coli, in Antibody Expression and Production (Al-Rubeai ed., 2011); and Therapeutic Monoclonal Antibodies: From Bench to Clinic (An ed., 2009).

It is, of course, contemplated that alternative methods, which are well known in the art, may be employed to prepare anti-LILRB1 and/or LILRB2 antibodies (e.g., anti-LILRB1 antibodies, anti-LILRB2 antibodies, or multispecific antibodies that bind to both LILRB1 and LILRB2). For instance, the appropriate amino acid sequence, or portions thereof, may be produced by direct peptide synthesis using solid-phase techniques (see, e.g., Stewart et al., Solid-Phase Peptide Synthesis (1969); and Merrifield, J. Am. Chem. Soc. 85:2149-54 (1963)). In vitro protein synthesis may be performed using manual techniques or by automation. Various portions of the anti-LILRB1 and/or LILRB2 antibody may be chemically synthesized separately and combined using chemical or enzymatic methods to produce the desired anti-LILRB1 and/or LILRB2 antibody. Alternatively, antibodies may be purified from cells or bodily fluids, such as milk, of a transgenic animal engineered to express the antibody, as disclosed, for example, in U.S. Pat. Nos. 5,545,807 and 5,827,690.

### Polyclonal Antibodies

Polyclonal antibodies are generally raised in animals by multiple subcutaneous (sc) or intraperitoneal (ip) injections of the relevant antigen and an adjuvant. It may be useful to conjugate the relevant antigen to a protein that is immunogenic in the species to be immunized, e.g., keyhole limpet hemocyanin (KLH), serum albumin, bovine thyroglobulin, or soybean trypsin inhibitor, using a bifunctional or derivatizing agent, e.g., maleimidobenzoyl sulfosuccinimide ester (conjugation through cysteine residues), N-hydroxysuccinimide (through lysine residues), glutaraldehyde, succinic anhydride, SOCl₂, or R¹N═C═NR, where R and R¹ are independently lower alkyl groups. Examples of adjuvants which may be employed include Freund's complete adjuvant and MPL-TDM adjuvant (monophosphoryl Lipid A, synthetic trehalose dicorynomycolate). The immunization protocol may be selected by one skilled in the art without undue experimentation.

For example, the animals are immunized against the antigen, immunogenic conjugates, or derivatives by combining, e.g., 100 μg or 5 μg of the protein or conjugate (for rabbits or mice, respectively) with 3 volumes of Freund's complete adjuvant and injecting the solution intradermally at multiple sites. One month later, the animals are boosted with ⅕ to 1/10 the original amount of peptide or conjugate in Freund's complete adjuvant by subcutaneous injection at multiple sites. Seven to fourteen days later, the animals are bled and the serum is assayed for antibody titer. Animals are boosted until the titer plateaus. Conjugates also can be made in recombinant cell culture as protein fusions. Also, aggregating agents such as alum are suitable to enhance the immune response.

### Monoclonal Antibodies

Monoclonal antibodies are obtained from a population of substantially homogeneous antibodies, i.e., the individual antibodies comprising the population are identical except for possible naturally occurring mutations and/or post-translational modifications (e.g., isomerizations, amidations) that may be present in minor amounts. Thus, the modifier “monoclonal” indicates the character of the antibody as not being a mixture of discrete antibodies.

For example, the monoclonal antibodies may be made using the hybridoma method first described by Kohler et al., Nature, 256:495 (1975), or may be made by recombinant DNA methods (U.S. Pat. No. 4,816,567).

In a further embodiment, antibodies can be isolated from antibody phage libraries generated using the techniques described in McCafferty et al., Nature, 348:552-554 (1990). Clackson et al., Nature, 352:624-628 (1991) and Marks et al., J. Mol. Biol., 222:581-597 (1991). Subsequent publications describe the production of high affinity (nM range) human antibodies by chain shuffling (Marks et al., Bio/Technology, 10:779-783 (1992)), as well as combinatorial infection and in vivo recombination as a strategy for constructing very large phage libraries (Waterhouse et al., Nucl. Acids Res., 21:2265-2266 (1993)). Thus, these techniques are viable alternatives to traditional monoclonal antibody hybridoma techniques for isolation of monoclonal antibodies.

The DNA also may be modified, for example, by substituting the coding sequence (U.S. Pat. No. 4,816,567; Morrison, et al., Proc. Natl Acad. Sci. USA, 81:6851 (1984)), or by covalently joining to the coding sequence all or part of the coding sequence for a non-immunoglobulin polypeptide. Such non-immunoglobulin polypeptides can be substituted to create a chimeric bivalent antibody comprising one antigen-combining site having specificity for an antigen and another antigen-combining site having specificity for a different antigen.

Chimeric or hybrid antibodies also may be prepared in vitro using known methods in synthetic protein chemistry, including those involving crosslinking agents. For example, immunotoxins may be constructed using a disulfide-exchange reaction or by forming a thioether bond. Examples of suitable reagents for this purpose include iminothiolate and methyl-4-mercaptobutyrimidate.

### Recombinant Production in Prokaryotic Cells

Polynucleic acid sequences encoding the antibodies of the present disclosure can be obtained using standard recombinant techniques. Desired polynucleic acid sequences may be isolated and sequenced from antibody producing cells such as hybridoma cells. Alternatively, polynucleotides can be synthesized using nucleotide synthesizer or PCR techniques. Once obtained, sequences encoding the polypeptides are inserted into a recombinant vector capable of replicating and expressing heterologous polynucleotides in prokaryotic hosts. Many vectors that are available and known in the art can be used for the purpose of the present disclosure. Selection of an appropriate vector will depend mainly on the size of the nucleic acids to be inserted into the vector and the particular host cell to be transformed with the vector. Each vector contains various components, depending on its function (amplification or expression of heterologous polynucleotide, or both) and its compatibility with the particular host cell in which it resides. The vector components generally include, but are not limited to, an origin of replication, a selection marker gene, a promoter, a ribosome binding site (RBS), a signal sequence, the heterologous nucleic acid insert and a transcription termination sequence.

In general, plasmid vectors containing replicon and control sequences which are derived from species compatible with the host cell are used in connection with these hosts. The vector ordinarily carries a replication site, as well as marking sequences which are capable of providing phenotypic selection in transformed cells. For example, E. coli is typically transformed using pBR322, a plasmid derived from an E. coli species. Examples of pBR322 derivatives used for expression of particular antibodies are described in detail in Carter et al., U.S. Pat. No. 5,648,237.

In addition, phage vectors containing replicon and control sequences that are compatible with the host microorganism can be used as transforming vectors in connection with these hosts. For example, bacteriophage such as GEM™-11 may be utilized in making a recombinant vector which can be used to transform susceptible host cells such as E. coli LE392.

The expression vector of the present application may comprise two or more promoter-cistron pairs, encoding each of the polypeptide components. A promoter is an untranslated regulatory sequence located upstream (5′) to a cistron that modulates its expression. Prokaryotic promoters typically fall into two classes, inducible and constitutive. Inducible promoter is a promoter that initiates increased levels of transcription of the cistron under its control in response to changes in the culture condition, e.g. the presence or absence of a nutrient or a change in temperature.

A large number of promoters recognized by a variety of potential host cells are well known. The selected promoter can be operably linked to cistron DNA encoding the present antibody by removing the promoter from the source DNA via restriction enzyme digestion and inserting the isolated promoter sequence into the vector of the present application. Both the native promoter sequence and many heterologous promoters may be used to direct amplification and/or expression of the target genes. In some embodiments, heterologous promoters are utilized, as they generally permit greater transcription and higher yields of expressed target gene as compared to the native target polypeptide promoter.

Promoters suitable for use with prokaryotic hosts include the PhoA promoter, the-galactamase and lactose promoter systems, a tryptophan (trp) promoter system and hybrid promoters such as the tac or the trc promoter. However, other promoters that are functional in bacteria (such as other known bacterial or phage promoters) are suitable as well. Their nucleic acid sequences have been published, thereby enabling a skilled worker operably to ligate them to cistrons encoding the target peptide (Siebenlist et al. Cell 20:269 (1980)) using linkers or adaptors to supply any required restriction sites.

In one aspect, each cistron within the recombinant vector comprises a secretion signal sequence component that directs translocation of the expressed polypeptides across a membrane. In general, the signal sequence may be a component of the vector, or it may be a part of the target polypeptide DNA that is inserted into the vector. The signal sequence selected for the purpose of this invention should be one that is recognized and processed (i.e. cleaved by a signal peptidase) by the host cell. For prokaryotic host cells that do not recognize and process the signal sequences native to the heterologous polypeptides, the signal sequence can be substituted by a prokaryotic signal sequence selected, for example, from the group consisting of the alkaline phosphatase, penicillinase, Ipp, or heat-stable enterotoxin II (STII) leaders, LamB, PhoE, PelB, OmpA and MBP.

In some embodiments, the production of the antibodies according to the present disclosure can occur in the cytoplasm of the host cell, and therefore does not require the presence of secretion signal sequences within each cistron. Certain host strains (e.g., the E. coli trxB⁻ strains) provide cytoplasm conditions that are favorable for disulfide bond formation, thereby permitting proper folding and assembly of expressed protein subunits.

Prokaryotic host cells suitable for expressing the antibodies of the present disclosure include Archaebacteria and Eubacteria, such as Gram-negative or Gram-positive organisms. Examples of useful bacteria include Escherichia (e.g., E. coli), Bacilli (e.g., B. subtilis), Enterobacteria, Pseudomonas species (e.g., P. aeruginosa), Salmonella typhimurium, Serratia marcescans, Klebsiella, Proteus, Shigella, Rhizobia, Vitreoscilla, or Paracoccus. In some embodiments, gram-negative cells are used. In one embodiment, E. coli cells are used as hosts. Examples of E. coli strains include strain W3110 (Bachmann, Cellular and Molecular Biology, vol. 2 (Washington, D.C.: American Society for Microbiology, 1987), pp. 1190-1219; ATCC Deposit No. 27,325) and derivatives thereof, including strain 33D3 having genotype W3110 AfhuA (AtonA) ptr3 lac Iq lacL8 AompT A (nmpc-fepE) degP41 kan^{R} (U.S. Pat. No. 5,639,635). Other strains and derivatives thereof, such as E. coli 294 (ATCC 31,446), E. coli B, E. coli 1776 (ATCC 31,537) and E. coli RV308 (ATCC 31,608) are also suitable. These examples are illustrative rather than limiting. Methods for constructing derivatives of any of the above-mentioned bacteria having defined genotypes are known in the art and described in, for example, Bass et al., Proteins, 8:309-314 (1990). It is generally necessary to select the appropriate bacteria taking into consideration replicability of the replicon in the cells of a bacterium. For example, E. coli, Serratia, or Salmonella species can be suitably used as the host when well known plasmids such as pBR322, pBR325, pACYC177, or pKN410 are used to supply the replicon.

Typically the host cell should secrete minimal amounts of proteolytic enzymes, and additional protease inhibitors may desirably be incorporated in the cell culture.

Host cells are transformed with the above-described expression vectors and cultured in conventional nutrient media modified as appropriate for inducing promoters, selecting transformants, or amplifying the genes encoding the desired sequences. Transformation means introducing DNA into the prokaryotic host so that the DNA is replicable, either as an extrachromosomal element or by chromosomal integrant. Depending on the host cell used, transformation is done using standard techniques appropriate to such cells. The calcium treatment employing calcium chloride is generally used for bacterial cells that contain substantial cell-wall barriers. Another method for transformation employs polyethylene glycol/DMSO. Yet another technique used is electroporation.

Prokaryotic cells used to produce the antibodies of the present application are grown in media known in the art and suitable for culture of the selected host cells. Examples of suitable media include luria broth (LB) plus necessary nutrient supplements. In some embodiments, the media also contains a selection agent, chosen based on the construction of the expression vector, to selectively permit growth of prokaryotic cells containing the expression vector. For example, ampicillin is added to media for growth of cells expressing ampicillin resistant gene.

Any necessary supplements besides carbon, nitrogen, and inorganic phosphate sources may also be included at appropriate concentrations introduced alone or as a mixture with another supplement or medium such as a complex nitrogen source. Optionally the culture medium may contain one or more reducing agents selected from the group consisting of glutathione, cysteine, cystamine, thioglycollate, dithioerythritol and dithiothreitol. The prokaryotic host cells are cultured at suitable temperatures and pHs.

If an inducible promoter is used in the expression vector of the present application, protein expression is induced under conditions suitable for the activation of the promoter. In one aspect of the present application, PhoA promoters are used for controlling transcription of the polypeptides. Accordingly, the transformed host cells are cultured in a phosphate-limiting medium for induction. Preferably, the phosphate-limiting medium is the C.R.A.P medium (see, e.g., Simmons et al., J. Immunol. Methods 263:133-147 (2002)). A variety of other inducers may be used, according to the vector construct employed, as is known in the art.

The expressed antibodies of the present disclosure are secreted into and recovered from the periplasm of the host cells. Protein recovery typically involves disrupting the microorganism, generally by such means as osmotic shock, sonication or lysis. Once cells are disrupted, cell debris or whole cells may be removed by centrifugation or filtration. The proteins may be further purified, for example, by affinity resin chromatography. Alternatively, proteins can be transported into the culture media and isolated therein. Cells may be removed from the culture and the culture supernatant being filtered and concentrated for further purification of the proteins produced. The expressed polypeptides can be further isolated and identified using commonly known methods such as polyacrylamide gel electrophoresis (PAGE) and Western blot assay.

Alternatively, protein production is conducted in large quantity by a fermentation process. Various large-scale fed-batch fermentation procedures are available for production of recombinant proteins. To improve the production yield and quality of the antibodies of the present disclosure, various fermentation conditions can be modified. For example, the chaperone proteins have been demonstrated to facilitate the proper folding and solubility of heterologous proteins produced in bacterial host cells. Chen et al. J Bio Chem 274:19601-19605 (1999); U.S. Pat. Nos. 6,083,715; 6,027,888; Bothmann and Pluckthun, J. Biol. Chem. 275:17100-17105 (2000); Ramm and Pluckthun, J. Biol. Chem. 275:17106-17113 (2000); Arie et al., Mol. Microbiol. 39:199-210 (2001).

To minimize proteolysis of expressed heterologous proteins (especially those that are proteolytically sensitive), certain host strains deficient for proteolytic enzymes can be used for the present invention, as described in, for example, U.S. Pat. Nos. 5,264,365; 5,508,192; Hara et al., Microbial Drug Resistance, 2:63-72 (1996). E. coli strains deficient for proteolytic enzymes and transformed with plasmids overexpressing one or more chaperone proteins may be used as host cells in the expression system encoding the antibodies of the present application.

The antibodies produced herein can be further purified to obtain preparations that are substantially homogeneous for further assays and uses. Standard protein purification methods known in the art can be employed. The following procedures are exemplary of suitable purification procedures: fractionation on immunoaffinity or ion-exchange columns, ethanol precipitation, reverse phase HPLC, chromatography on silica or on a cation-exchange resin such as DEAE, chromatofocusing, SDS-PAGE, ammonium sulfate precipitation, and gel filtration using, for example, Sephadex G-75. Protein A immobilized on a solid phase for example can be used in some embodiments for immunoaffinity purification of binding molecules of the present disclosure. The solid phase to which Protein A is immobilized is preferably a column comprising a glass or silica surface, more preferably a controlled pore glass column or a silicic acid column. In some embodiments, the column has been coated with a reagent, such as glycerol, in an attempt to prevent nonspecific adherence of contaminants. The solid phase is then washed to remove contaminants non-specifically bound to the solid phase. Finally the antibodies of interest is recovered from the solid phase by elution.

### Recombinant Production in Eukaryotic Cells

For eukaryotic expression, the vector components generally include, but are not limited to, one or more of the following, a signal sequence, an origin of replication, one or more marker genes, and enhancer element, a promoter, and a transcription termination sequence.

A vector for use in a eukaryotic host may also an insert that encodes a signal sequence or other polypeptide having a specific cleavage site at the N-terminus of the mature protein or polypeptide. The heterologous signal sequence selected preferably is one that is recognized and processed (i.e., cleaved by a signal peptidase) by the host cell. In mammalian cell expression, mammalian signal sequences as well as viral secretory leaders, for example, the herpes simplex gD signal, are available. The DNA for such precursor region can be ligated in reading frame to DNA encoding the antibodies of the present application.

Generally, the origin of replication component is not needed for mammalian expression vectors (the SV40 origin may typically be used only because it contains the early promoter).

Expression and cloning vectors may contain a selection gene, also termed a selectable marker. Selection genes may encode proteins that confer resistance to antibiotics or other toxins, e.g., ampicillin, neomycin, methotrexate, or tetracycline; complement auxotrophic deficiencies; or supply critical nutrients not available from complex media.

One example of a selection scheme utilizes a drug to arrest growth of a host cell. Those cells that are successfully transformed with a heterologous gene produce a protein conferring drug resistance and thus survive the selection regimen. Examples of such dominant selection use the drugs neomycin, mycophenolic acid and hygromycin.

Another example of suitable selectable markers for mammalian cells are those that enable the identification of cells competent to take up nucleic acid encoding the antibodies of the present application. For example, cells transformed with the DHFR selection gene are first identified by culturing all of the transformants in a culture medium that contains methotrexate (Mtx), a competitive antagonist of DHFR. An exemplary appropriate host cell when wild-type DHFR is employed is the Chinese hamster ovary (CHO) cell line deficient in DHFR activity. Alternatively, host cells (particularly wild-type hosts that contain endogenous DHFR) transformed or co-transformed with the polypeptide encoding-DNA sequences, wild-type DHFR protein, and another selectable marker such as aminoglycoside 3′-phosphotransferase (APH) can be selected by cell growth in medium containing a selection agent for the selectable marker such as an aminoglycosidic antibiotic.

Expression and cloning vectors usually contain a promoter that is recognized by the host organism and is operably linked to the nucleic acid encoding the desired polypeptide sequences. Eukaryotic genes have an AT-rich region located approximately 25 to 30 based upstream from the site where transcription is initiated. Another sequence found 70 to 80 bases upstream from the start of the transcription of many genes may be included. The 3′ end of most eukaryotic may be the signal for addition of the poly A tail to the 3′ end of the coding sequence. All of these sequences may be inserted into eukaryotic expression vectors.

Polypeptide transcription from vectors in mammalian host cells can be controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., the actin promoter or an immunoglobulin promoter, from heat-shock promoters, provided such promoters are compatible with the host cell systems.

Transcription of a DNA encoding the antibodies of the present disclosure by higher eukaryotes is often increased by inserting an enhancer sequence into the vector. Many enhancer sequences are now known from mammalian genes (globin, elastase, albumin, α-fetoprotein, and insulin). Examples include the SV40 enhancer on the late side of the replication origin (bp 100-270), the cytomegalovirus early promoter enhancer, the polyoma enhancer on the late side of the replication origin, and adenovirus enhancers. See also Yaniv, Nature 297:17-18 (1982) on enhancing elements for activation of eukaryotic promoters. The enhancer may be spliced into the vector at a position 5′ or 3′ to the polypeptide encoding sequence, but is preferably located at a site 5′ from the promoter.

Expression vectors used in eukaryotic host cells (yeast, fungi, insect, plant, animal, human, or nucleated cells from other multicellular organisms) also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the polypeptide-encoding mRNA. One useful transcription termination component is the bovine growth hormone polyadenylation region.

Suitable host cells for cloning or expressing the DNA in the vectors herein include higher eukaryote cells described herein, including vertebrate host cells. Propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/−DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TR1 cells (Mather et al., Annals N.Y. Acad. Sci. 383:44-68 (1982)); MRC 5 cells; FS4 cells; and a human hepatoma line (Hep G2).

Host cells can be transformed with the above-described expression or cloning vectors for antibodies production and cultured in conventional nutrient media modified as appropriate for inducing promoters, selecting transformants, or amplifying the genes encoding the desired sequences.

The host cells used to produce the antibodies of the present application may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

When using recombinant techniques, the antibodies can be produced intracellularly, in the periplasmic space, or directly secreted into the medium. If the antibody is produced intracellularly, as a first step, the particulate debris, either host cells or lysed fragments, are removed, for example, by centrifugation or ultrafiltration. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The protein composition prepared from the cells can be purified using, for example, hydroxylapatite chromatography, gel electrophoresis, dialysis, and affinity chromatography, with affinity chromatography being the preferred purification technique. The matrix to which the affinity ligand is attached is most often agarose, but other matrices are available. Mechanically stable matrices such as controlled pore glass or poly(styrene-divinyl)benzene allow for faster flow rates and shorter processing times than can be achieved with agarose. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered. Following any preliminary purification step(s), the mixture comprising the antibody of interest and contaminants may be subjected to low pH hydrophobic interaction chromatography.

### 5.5. Pharmaceutical Compositions

In one aspect, the present disclosure further provides pharmaceutical compositions comprising at least one antibody or antigen binding fragment thereof of the present disclosure. In some embodiments, a pharmaceutical composition comprises therapeutically effective amount of an antibody or antigen binding fragment thereof provided herein and a pharmaceutically acceptable excipient.

Pharmaceutical compositions comprising an antibody or antigen binding fragment thereof are prepared for storage by mixing the fusion protein having the desired degree of purity with optional physiologically acceptable excipients (see, e.g., Remington, Remington's Pharmaceutical Sciences (18th ed. 1980)) in the form of aqueous solutions or lyophilized or other dried forms.

The antibody or antigen binding fragment thereof of the present disclosure may be formulated in any suitable form for delivery to a target cell/tissue, e.g., as microcapsules or macroemulsions (Remington, supra; Park et al., 2005, Molecules 10:146-61; Malik et al., 2007, Curr. Drug. Deliv. 4:141-51), as sustained release formulations (Putney and Burke, 1998, Nature Biotechnol. 16:153-57), or in liposomes (Maclean et al., 1997, Int. J. Oncol. 11:325-32; Kontermann, 2006, Curr. Opin. Mol. Ther. 8:39-45).

An antibody or antigen binding fragment thereof provided herein can also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles, and nanocapsules) or in macroemulsions. Such techniques are disclosed, for example, in Remington, supra.

Various compositions and delivery systems are known and can be used with an antibody or antigen binding fragment thereof as described herein, including, but not limited to, encapsulation in liposomes, microparticles, microcapsules, recombinant cells capable of expressing the antibody or antigen binding fragment thereof, receptor-mediated endocytosis (see, e.g., Wu and Wu, 1987, J. Biol. Chem. 262:4429-32), construction of a nucleic acid as part of a retroviral or other vector, etc. In another embodiment, a composition can be provided as a controlled release or sustained release system. In one embodiment, a pump may be used to achieve controlled or sustained release (see, e.g., Langer, supra; Sefton, 1987, Crit. Ref. Biomed. Eng. 14:201-40; Buchwald et al., 1980, Surgery 88:507-16; and Saudek et al., 1989, N. Engl. J. Med. 321:569-74). In another embodiment, polymeric materials can be used to achieve controlled or sustained release of a prophylactic or therapeutic agent (e.g., an antibody or antigen binding fragment thereof as described herein) or a composition provided herein (see, e.g., Medical Applications of Controlled Release (Langer and Wise eds., 1974); Controlled Drug Bioavailability, Drug Product Design and Performance (Smolen and Ball eds., 1984); Ranger and Peppas, 1983, J. Macromol. Sci. Rev. Macromol. Chem. 23:61-126; Levy et al., 1985, Science 228:190-92; During et al., 1989, Ann. Neurol. 25:351-56; Howard et al., 1989, J. Neurosurg. 71:105-12; U.S. Pat. Nos. 5,679,377; 5,916,597; 5,912,015; 5,989,463; and 5,128,326; PCT Publication Nos. WO 99/15154 and WO 99/20253). Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In one embodiment, the polymer used in a sustained release formulation is inert, free of leachable impurities, stable on storage, sterile, and biodegradable.

In yet another embodiment, a controlled or sustained release system can be placed in proximity of a particular target tissue, for example, the nasal passages or lungs, thus requiring only a fraction of the systemic dose (see, e.g., Goodson, Medical Applications of Controlled Release Vol. 2, 115-38 (1984)). Controlled release systems are discussed, for example, by Langer, 1990, Science 249:1527-33. Any technique known to one of skill in the art can be used to produce sustained release formulations comprising one or more antibody or antigen binding fragment thereof as described herein (see, e.g., U.S. Pat. No. 4,526,938, PCT publication Nos. WO 91/05548 and WO 96/20698, Ning et al., 1996, Radiotherapy & Oncology 39:179-89; Song et al., 1995, PDA J. of Pharma. Sci. & Tech. 50:372-97; Cleek et al., 1997, Pro. Int'l. Symp. Control. Rel. Bioact. Mater. 24:853-54; and Lam et al., 1997, Proc. Int'l. Symp. Control Rel. Bioact. Mater. 24:759-60).

### 5.6. Methods of Using the Antibodies

In another aspect, provided herein are methods for using the binding agents (e.g. the anti-LILRB1 antibodies, the anti-LILRB2 antibodies, or the multispecific antibodies that bind to both LILRB1 and LILRB2) or the composition provided herein.

The binding agents (e.g., the anti-LILRB1 antibodies, the anti-LILRB2 antibodies, or the multispecific antibodies that bind to both LILRB1 and LILRB2) of the disclosure are useful in a variety of applications including, but not limited to, therapeutic treatment methods, such as treatment of cancer. In some embodiments, the therapeutic treatment methods comprise immunotherapy for cancer. In some embodiments, a binding agent described herein is useful for activating, promoting, increasing, and/or enhancing an immune response to cancer or cancer cells. In some embodiments, a binding agent described herein is useful for activating, promoting, increasing, and/or enhancing an immune response to a tumor or tumor cells. The methods of use may be in vitro, ex vivo, or in vivo methods.

In some embodiments, provided herein is a method of inhibiting interaction between LILRB1 (for example, expressed on and/or in a first cell) and ligands of LILRB1 (for example, expressed on and/or in a second cell), comprising contacting LILRB1 (for example, the first cell expressing LILRB1) with a binding agent (e.g., an antibody or fragment thereof) provided herein. In some embodiments, provided herein is a use of the binding agent provided herein for inhibiting interaction between LILRB1 (for example, expressed on and/or in a first cell) and ligands of LILRB1 (for example, expressed on and/or in a second cell), wherein the use comprises contacting LILRB1 (for example, the first cell expressing LILRB1) with a multispecific binding agent (e.g., the antibody or fragment thereof) provided herein. In some embodiments, the ligands are MHC I molecules. In some embodiments, the ligand is HLA-A2. In some embodiments, the ligand is HLA-G.

As used herein, the term “inhibit” means decrease or reduce. For example, in some embodiments, the binding agent provided herein inhibits the interaction between LILRB1 and ligands of LILRB1 by 10%-99%. In other embodiments, the binding agent provided herein inhibits the interaction between LILRB1 and ligands of LILRB1 100% (i.e., completely abolish the interaction as measured by an assay). In some embodiments, the binding agent provided herein inhibits the interaction between LILRB1 and ligands of LILRB1 by at least 10%. In some embodiments, the binding agent provided herein inhibits the interaction between LILRB1 and ligands of LILRB1 by at least 20%. In some embodiments, the binding agent provided herein inhibits the interaction between LILRB1 and ligands of LILRB1 by at least 30%. In some embodiments, the binding agent provided herein inhibits the interaction between LILRB1 and ligands of LILRB1 by at least 40%. In some embodiments, the binding agent provided herein inhibits the interaction between LILRB1 and ligands of LILRB1 by at least 50%. In some embodiments, the binding agent provided herein inhibits the interaction between LILRB1 and ligands of LILRB1 by at least 60%. In some embodiments, the binding agent provided herein inhibits the interaction between LILRB1 and ligands of LILRB1 by at least 70%. In some embodiments, the binding agent provided herein inhibits the interaction between LILRB1 and ligands of LILRB1 by at least 80%. In some embodiments, the binding agent provided herein inhibits the interaction between LILRB1 and ligands of LILRB1 by at least 90%. In some embodiments, the binding agent provided herein inhibits the interaction between LILRB1 and ligands of LILRB1 by about 10%-90%. In some embodiments, the binding agent provided herein inhibits the interaction between LILRB1 and ligands of LILRB1 by about 20%-80%. In some embodiments, the binding agent provided herein inhibits the interaction between LILRB1 and ligands of LILRB1 by about 30%-70%. In some embodiments, the binding agent provided herein inhibits the interaction between LILRB1 and ligands of LILRB1 by about 40%-60%. In some embodiments, the LILRB1 and the ligands of LILRB1 are expressed on different cells. In some embodiments, the LILRB1 cell is expressed on a first cell, such as an immune cell. In some embodiments, the immune cell is an NK cell. In some embodiments, the immune cell is a monocyte. In some embodiments, the immune cell is a macrophage. In some embodiments, the immune cell is a T cell. In some embodiments, the T cell is a cytotoxic T cell such as a CD8+ T cell. In some embodiments, the immune cell is a dendritic cell.

As used herein, the term “inhibit” means decrease or reduce. For example, in some embodiments, the binding agent provided herein inhibits the interaction between LILRB1 and HLA-A2 by 10%-99%. In other embodiments, the binding agent provided herein inhibits the interaction between LILRB1 and HLA-A2 100% (i.e., completely abolish the interaction as measured by an assay). In some embodiments, the binding agent provided herein inhibits the interaction between LILRB1 and HLA-A2 by at least 10%. In some embodiments, the binding agent provided herein inhibits the interaction between LILRB1 and HLA-A2 by at least 20%. In some embodiments, the binding agent provided herein inhibits the interaction between LILRB1 and HLA-A2 by at least 30%. In some embodiments, the binding agent provided herein inhibits the interaction between LILRB1 and HLA-A2 by at least 40%. In some embodiments, the binding agent provided herein inhibits the interaction between LILRB1 and HLA-A2 by at least 50%. In some embodiments, the binding agent provided herein inhibits the interaction between LILRB1 and HLA-A2 by at least 60%. In some embodiments, the binding agent provided herein inhibits the interaction between LILRB1 and HLA-A2 by at least 70%. In some embodiments, the binding agent provided herein inhibits the interaction between LILRB1 and HLA-A2 by at least 80%. In some embodiments, the binding agent provided herein inhibits the interaction between LILRB1 and HLA-A2 by at least 90%. In some embodiments, the binding agent provided herein inhibits the interaction between LILRB1 and HLA-A2 by about 10%-90%. In some embodiments, the binding agent provided herein inhibits the interaction between LILRB1 and HLA-A2 by about 20%-80%. In some embodiments, the binding agent provided herein inhibits the interaction between LILRB1 and HLA-A2 by about 30%-70%. In some embodiments, the binding agent provided herein inhibits the interaction between LILRB1 and HLA-A2 by about 40%-60%. In some embodiments, the LILRB1 and the HLA-A2 are expressed on different cells. In some embodiments, the LILRB1 cell is expressed on a first cell, such as an immune cell. In some embodiments, the immune cell is an NK cell. In some embodiments, the immune cell is a monocyte. In some embodiments, the immune cell is a macrophage. In some embodiments, the immune cell is a T cell. In some embodiments, the T cell is a cytotoxic T cell such as a CD8+ T cell. In some embodiments, the immune cell is a dendritic cell.

As used herein, the term “inhibit” means decrease or reduce. For example, in some embodiments, the binding agent provided herein inhibits the interaction between LILRB1 and HLA-G by 10%-99%. In other embodiments, the binding agent provided herein inhibits the interaction between LILRB1 and HLA-G 100% (i.e., completely abolish the interaction as measured by an assay). In some embodiments, the binding agent provided herein inhibits the interaction between LILRB1 and HLA-G by at least 10%. In some embodiments, the binding agent provided herein inhibits the interaction between LILRB1 and HLA-G by at least 20%. In some embodiments, the binding agent provided herein inhibits the interaction between LILRB1 and HLA-G by at least 30%. In some embodiments, the binding agent provided herein inhibits the interaction between LILRB1 and HLA-G by at least 40%. In some embodiments, the binding agent provided herein inhibits the interaction between LILRB1 and HLA-G by at least 50%. In some embodiments, the binding agent provided herein inhibits the interaction between LILRB1 and HLA-G by at least 60%. In some embodiments, the binding agent provided herein inhibits the interaction between LILRB1 and HLA-G by at least 70%. In some embodiments, the binding agent provided herein inhibits the interaction between LILRB1 and HLA-G by at least 80%. In some embodiments, the binding agent provided herein inhibits the interaction between LILRB1 and HLA-G by at least 90%. In some embodiments, the binding agent provided herein inhibits the interaction between LILRB1 and HLA-G by about 10%-90%. In some embodiments, the binding agent provided herein inhibits the interaction between LILRB1 and HLA-G by about 20%-80%. In some embodiments, the binding agent provided herein inhibits the interaction between LILRB1 and HLA-G by about 30%-70%. In some embodiments, the binding agent provided herein inhibits the interaction between LILRB1 and HLA-G by about 40%-60%. In some embodiments, the LILRB1 and the HLA-G are expressed on different cells. In some embodiments, the LILRB1 cell is expressed on a first cell, such as an immune cell. In some embodiments, the immune cell is an NK cell. In some embodiments, the immune cell is a monocyte. In some embodiments, the immune cell is a macrophage. In some embodiments, the immune cell is a T cell. In some embodiments, the T cell is a cytotoxic T cell such as a CD8+ T cell. In some embodiments, the immune cell is a dendritic cell.

In some embodiments, provided herein is a method of inhibiting interaction between LILRB2 (for example, expressed on and/or in a first cell) and ligands of LILRB2 (for example, expressed on and/or in a second cell), comprising contacting LILRB2 (for example, the first cell expressing LILRB2) with a binding agent (e.g., an antibody or fragment thereof) provided herein. In some embodiments, provided herein is a use of the binding agent provided herein for inhibiting interaction between LILRB2 (for example, expressed on and/or in a first cell) and ligands of LILRB2 (for example, expressed on and/or in a second cell), wherein the use comprises contacting LILRB2 (for example, the first cell expressing LILRB2) with a multispecific binding agent (e.g., the antibody or fragment thereof) provided herein. In some embodiments, the ligands are MHC I molecules. In some embodiments, the ligand is HLA-A2. In some embodiments, the ligand is HLA-G. In some embodiments, the ligand is ANGPTLs.

As used herein, the term “inhibit” means decrease or reduce. For example, in some embodiments, the binding agent provided herein inhibits the interaction between LILRB2 and ligands of LILRB2 by 10%-99%. In other embodiments, the binding agent provided herein inhibits the interaction between LILRB2 and ligands of LILRB2 100% (i.e., completely abolish the interaction as measured by an assay). In some embodiments, the binding agent provided herein inhibits the interaction between LILRB2 and ligands of LILRB2 by at least 10%. In some embodiments, the binding agent provided herein inhibits the interaction between LILRB2 and ligands of LILRB2 by at least 20%. In some embodiments, the binding agent provided herein inhibits the interaction between LILRB2 and ligands of LILRB2 by at least 30%. In some embodiments, the binding agent provided herein inhibits the interaction between LILRB2 and ligands of LILRB2 by at least 40%. In some embodiments, the binding agent provided herein inhibits the interaction between LILRB2 and ligands of LILRB2 by at least 50%. In some embodiments, the binding agent provided herein inhibits the interaction between LILRB2 and ligands of LILRB2 by at least 60%. In some embodiments, the binding agent provided herein inhibits the interaction between LILRB2 and ligands of LILRB2 by at least 70%. In some embodiments, the binding agent provided herein inhibits the interaction between LILRB2 and ligands of LILRB2 by at least 80%. In some embodiments, the binding agent provided herein inhibits the interaction between LILRB2 and ligands of LILRB2 by at least 90%. In some embodiments, the binding agent provided herein inhibits the interaction between LILRB2 and ligands of LILRB2 by about 10%-90%. In some embodiments, the binding agent provided herein inhibits the interaction between LILRB2 and ligands of LILRB2 by about 20%-80%. In some embodiments, the binding agent provided herein inhibits the interaction between LILRB2 and ligands of LILRB2 by about 30%-70%. In some embodiments, the binding agent provided herein inhibits the interaction between LILRB2 and ligands of LILRB2 by about 40%-60%. In some embodiments, the LILRB2 and the ligands of LILRB2 are expressed on different cells. In some embodiments, the LILRB2 cell is expressed on a first cell, such as an immune cell. In some embodiments, the immune cell is an NK cell. In some embodiments, the immune cell is a monocyte. In some embodiments, the immune cell is a macrophage. In some embodiments, the immune cell is a T cell. In some embodiments, the T cell is a cytotoxic T cell such as a CD8+ T cell. In some embodiments, the immune cell is a dendritic cell.

As used herein, the term “inhibit” means decrease or reduce. For example, in some embodiments, the binding agent provided herein inhibits the interaction between LILRB2 and HLA-A2 by 10%-99%. In other embodiments, the binding agent provided herein inhibits the interaction between LILRB2 and HLA-A2 100% (i.e., completely abolish the interaction as measured by an assay). In some embodiments, the binding agent provided herein inhibits the interaction between LILRB2 and HLA-A2 by at least 10%. In some embodiments, the binding agent provided herein inhibits the interaction between LILRB2 and HLA-A2 by at least 20%. In some embodiments, the binding agent provided herein inhibits the interaction between LILRB2 and HLA-A2 by at least 30%. In some embodiments, the binding agent provided herein inhibits the interaction between LILRB2 and HLA-A2 by at least 40%. In some embodiments, the binding agent provided herein inhibits the interaction between LILRB2 and HLA-A2 by at least 50%. In some embodiments, the binding agent provided herein inhibits the interaction between LILRB2 and HLA-A2 by at least 60%. In some embodiments, the binding agent provided herein inhibits the interaction between LILRB2 and HLA-A2 by at least 70%. In some embodiments, the binding agent provided herein inhibits the interaction between LILRB2 and HLA-A2 by at least 80%. In some embodiments, the binding agent provided herein inhibits the interaction between LILRB2 and HLA-A2 by at least 90%. In some embodiments, the binding agent provided herein inhibits the interaction between LILRB2 and HLA-A2 by about 10%-90%. In some embodiments, the binding agent provided herein inhibits the interaction between LILRB2 and HLA-A2 by about 20%-80%. In some embodiments, the binding agent provided herein inhibits the interaction between LILRB2 and HLA-A2 by about 30%-70%. In some embodiments, the binding agent provided herein inhibits the interaction between LILRB2 and HLA-A2 by about 40%-60%. In some embodiments, the LILRB2 and the HLA-A2 are expressed on different cells. In some embodiments, the LILRB2 cell is expressed on a first cell, such as an immune cell. In some embodiments, the immune cell is an NK cell. In some embodiments, the immune cell is a monocyte. In some embodiments, the immune cell is a macrophage. In some embodiments, the immune cell is a T cell. In some embodiments, the T cell is a cytotoxic T cell such as a CD8+ T cell. In some embodiments, the immune cell is a dendritic cell.

As used herein, the term “inhibit” means decrease or reduce. For example, in some embodiments, the binding agent provided herein inhibits the interaction between LILRB2 and ANGPTLS by 10%-99%. In other embodiments, the binding agent provided herein inhibits the interaction between LILRB2 and ANGPTLS 100% (i.e., completely abolish the interaction as measured by an assay). In some embodiments, the binding agent provided herein inhibits the interaction between LILRB2 and ANGPTLS by at least 10%. In some embodiments, the binding agent provided herein inhibits the interaction between LILRB2 and ANGPTLS by at least 20%. In some embodiments, the binding agent provided herein inhibits the interaction between LILRB2 and ANGPTLS by at least 30%. In some embodiments, the binding agent provided herein inhibits the interaction between LILRB2 and ANGPTLS by at least 40%. In some embodiments, the binding agent provided herein inhibits the interaction between LILRB2 and ANGPTLS by at least 50%. In some embodiments, the binding agent provided herein inhibits the interaction between LILRB2 and ANGPTLS by at least 60%. In some embodiments, the binding agent provided herein inhibits the interaction between LILRB2 and ANGPTLS by at least 70%. In some embodiments, the binding agent provided herein inhibits the interaction between LILRB2 and ANGPTLS by at least 80%. In some embodiments, the binding agent provided herein inhibits the interaction between LILRB2 and ANGPTLS by at least 90%. In some embodiments, the binding agent provided herein inhibits the interaction between LILRB2 and ANGPTLS by about 10%-90%. In some embodiments, the binding agent provided herein inhibits the interaction between LILRB2 and ANGPTLS by about 20%-80%. In some embodiments, the binding agent provided herein inhibits the interaction between LILRB2 and ANGPTLS by about 30%-70%. In some embodiments, the binding agent provided herein inhibits the interaction between LILRB2 and ANGPTLS by about 40%-60%. In some embodiments, the LILRB2 and the ANGPTLS are expressed on different cells. In some embodiments, the LILRB2 cell is expressed on a first cell, such as an immune cell. In some embodiments, the immune cell is an NK cell. In some embodiments, the immune cell is a monocyte. In some embodiments, the immune cell is a macrophage. In some embodiments, the immune cell is a T cell. In some embodiments, the T cell is a cytotoxic T cell such as a CD8+ T cell. In some embodiments, the immune cell is a dendritic cell.

As used herein, the term “inhibit” means decrease or reduce. For example, in some embodiments, the binding agent provided herein inhibits the interaction between LILRB2 and HLA-G by 10%-99%. In other embodiments, the binding agent provided herein inhibits the interaction between LILRB2 and HLA-G 100% (i.e., completely abolish the interaction as measured by an assay). In some embodiments, the binding agent provided herein inhibits the interaction between LILRB2 and HLA-G by at least 10%. In some embodiments, the binding agent provided herein inhibits the interaction between LILRB2 and HLA-G by at least 20%. In some embodiments, the binding agent provided herein inhibits the interaction between LILRB2 and HLA-G by at least 30%. In some embodiments, the binding agent provided herein inhibits the interaction between LILRB2 and HLA-G by at least 40%. In some embodiments, the binding agent provided herein inhibits the interaction between LILRB2 and HLA-G by at least 50%. In some embodiments, the binding agent provided herein inhibits the interaction between LILRB2 and HLA-G by at least 60%. In some embodiments, the binding agent provided herein inhibits the interaction between LILRB2 and HLA-G by at least 70%. In some embodiments, the binding agent provided herein inhibits the interaction between LILRB2 and HLA-G by at least 80%. In some embodiments, the binding agent provided herein inhibits the interaction between LILRB2 and HLA-G by at least 90%. In some embodiments, the binding agent provided herein inhibits the interaction between LILRB2 and HLA-G by about 10%-90%. In some embodiments, the binding agent provided herein inhibits the interaction between LILRB2 and HLA-G by about 20%-80%. In some embodiments, the binding agent provided herein inhibits the interaction between LILRB2 and HLA-G by about 30%-70%. In some embodiments, the binding agent provided herein inhibits the interaction between LILRB2 and HLA-G by about 40%-60%. In some embodiments, the LILRB2 and the HLA-G are expressed on different cells. In some embodiments, the LILRB2 cell is expressed on a first cell, such as an immune cell. In some embodiments, the immune cell is an NK cell. In some embodiments, the immune cell is a monocyte. In some embodiments, the immune cell is a macrophage. In some embodiments, the immune cell is a T cell. In some embodiments, the T cell is a cytotoxic T cell such as a CD8+ T cell. In some embodiments, the immune cell is a dendritic cell.

An immune cell is a cell in immune system and can be a cell of lymphoid lineage. Non-limiting examples of cells of lymphoid lineage include neutrophils, eosinophils, basophils, mast cells, monocytes, macrophages, dendritic cells, natural killer (NK) cells, and lymphocytes (B cells and T cells). T cells are a type of lymphocytes and can be characterized by expressing T cell receptors (TCRs). T cells play a central role in the adaptive immune response. T cell subtypes have a variety of important functions in controlling and shaping the immune response. For example, cytotoxic T cells (also called cytotoxic T lymphocyte and killer T cell) are T lymphocytes that kill certain cells, e.g., cancer cells, cells that are infected by intracellular pathogens (such as viruses or bacteria), or cells that are damaged in other ways. Most cytotoxic T cells express T-cell receptors (TCRs) that can recognize a specific antigen. CD8+ T cells are a subpopulation of MHC class I-restricted T cell and are mediators of adaptive immunity, which are important for killing cancerous or virally infected cells. NK cells are a type of cytotoxic lymphocytes critical to the innate immune system that belong to the family of innate lymphoid cells (ILC). In some embodiments, NK cells can be identified by the presence of CD56 and the absence of CD3 (CD56+, CD3−). NK cells have the ability to recognize and kill stressed cells in the absence of antibodies and MHC, allowing for a much faster immune reaction.

In some embodiments, the HLA-A2 is expressed on a second cell, such as a cancer or tumor cell. In some embodiments, the HLA-G is expressed on a second cell, such as a cancer or tumor cell. In some embodiments, the ANGPTLs is expressed on a second cell, such as a cancer or tumor cell.

In other embodiments, provided herein is a method of preventing suppression of an immune cell, e.g., a suppression mediated by the interaction between LILRB1 expressed on the immune cell with ligands of LILRB1 (e.g., expressed on a cancer or tumor cell), and/or a suppression mediated by the interaction between LILRB2 (e.g., expressed on the immune cell) and ligands of LILRB2 (e.g., expressed on a cancer or tumor cell). In yet other embodiments, provided herein is a method of activating a response mediated by an immune cell, e.g., an anti-tumor response. In some embodiments, the method comprises contacting the immune cell with a binding agent (e.g., an anti-LILRB1 antibody, an anti-LILRB2 antibody, or a multispecific antibody that binds to both LILRB1 and LILRB2) provided herein. In some embodiments, provided herein is a use of the binding agent provided herein for preventing suppression of an immune cell or activating a response mediated by an immune cell. In some embodiments, the immune cell is an NK cell. In some embodiments, the immune cell is a monocyte. In some embodiments, the immune cell is a macrophage. In some embodiments, the immune cell is a T cell. In some embodiments, the T cell is a cytotoxic T cell such as a CD8+ T cell. In some embodiments, the immune cell is a dendritic cell. In some embodiments, the immune cell expresses LILRB1. Additionally or alternatively, the immune cell expresses LILRB2. In some embodiments, the immune cell mediates an anti-cancer/tumor response. In further embodiments, the cancer or tumor cell expresses HLA-A2. Additionally or alternatively, the cancer or tumor cell expresses HLA-G. In further embodiments, the cancer or tumor cell expresses ANGPTLs.

The present disclosure provides methods for activating an immune response in a subject using a binding agent described herein (e.g. an anti-LILRB1 antibody, an anti-LILRB2 antibody, or a multispecific antibody that binds to both LILRB1 and LILRB2). In some embodiments, the disclosure provides methods for promoting an immune response in a subject using a binding agent described herein. In some embodiments, the disclosure provides methods for increasing an immune response in a subject using a binding agent described herein. In some embodiments, the disclosure provides methods for enhancing an immune response in a subject using a binding agent described herein. In some embodiments, the activating, promoting, increasing, and/or enhancing of an immune response comprises stimulating myeloid cells. In some embodiments, the activating, promoting, increasing, and/or enhancing of an immune response comprises stimulating monocytes. In some embodiments, the activating, promoting, increasing, and/or enhancing of an immune response comprises stimulating macrophages. In some embodiments, the activating, promoting, increasing, and/or enhancing of an immune response comprises enhancing macrophage phagocytosis. In some embodiments, the activating, promoting, increasing, and/or enhancing of an immune response comprises stimulating dendritic cells. In some embodiments, the activating, promoting, increasing, and/or enhancing of an immune response comprises enhancing FcR-mediated dendritic cell activity. In some embodiments, the activating, promoting, increasing, and/or enhancing of an immune response comprises increasing cell-mediated immunity. In some embodiments, the activating, promoting, increasing, and/or enhancing of an immune response comprises increasing effector T-cell activity. In some embodiments, the activating, promoting, increasing, and/or enhancing of an immune response comprises increasing CD8+ T cell activity. In some embodiments, the activating, promoting, increasing, and/or enhancing of an immune response comprises increasing NK cell activity. In some embodiments, the activating, promoting, increasing, and/or enhancing of an immune response comprises enhancing NK cell activity. In some embodiments, the immune response is a result of antigenic stimulation. In some embodiments, the antigenic stimulation is a tumor cell. In some embodiments, the antigenic stimulation is cancer. In some embodiments, the activating, promoting, increasing, and/or enhancing of an immune response comprises enhancing cytokine release by immune cells. In some embodiments, the activating, promoting, increasing, and/or enhancing of an immune response comprises enhancing TNFα release by immune cells. In some embodiments, the activating, promoting, increasing, and/or enhancing of an immune response comprises enhancing IFN-γ release by immune cells. In some embodiments, the antibody described herein enhancing anti-tumor immunity.

### Therapeutic Uses and Methods of Treatment

In some embodiments, the binding agents (e.g. the anti-LILRB1 antibodies, the anti-LILRB2 antibodies, or the multispecific antibodies that bind to both LILRB1 and LILRB2) described herein are useful in compositions and in methods of treating a disease or disorder. Thus, in some embodiments, provided herein is a method for treating a disease or disorder in a subject comprising administering the binding agent or the pharmaceutical composition provided herein to the subject. In other embodiments, provided herein is a use of the binding agent or pharmaceutical composition provided herein for treating a disease or disorder in a subject. In other embodiments, provided herein is a binding agent or pharmaceutical composition provided herein for use in the manufacture of a medicament for the treatment of a disease or disorder.

In some embodiments, the treatment provided herein includes alleviating one or more symptoms associated with a disease or disorder (e.g., a cancer or a tumor).

Thus, in some embodiments, provided herein is a method for alleviating one or more symptoms associated with a cancer or a tumor in a subject comprising administering to the subject a binding agent (e.g., an antibody) described herein or a pharmaceutical composition described herein. In some embodiments, provided herein is a binding agent (e.g., an antibody) described herein or a pharmaceutical composition described herein for use in alleviating one or more symptoms associated with a cancer or a tumor in a subject. In some embodiments, provided herein is a use of a binding agent (e.g., an antibody) described herein or a pharmaceutical composition described herein in the manufacture of a medicament for alleviating one or more symptoms associated with a cancer or a tumor in a subject.

In some embodiments, the treatment provided herein includes decreasing tumor size in a subject with a tumor. Thus, in some embodiments, described herein is a method for decreasing tumor size in a subject with a tumor comprising administering to the subject a binding agent (e.g., an antibody) described herein or a pharmaceutical composition described herein. In some embodiments, provided herein is a binding agent (e.g., an antibody) described herein or a pharmaceutical composition described herein for use in decreasing tumor size in a subject with a tumor. In some embodiments, provided herein is a use of a binding agent (e.g., an antibody) described herein or a pharmaceutical composition described herein in the manufacture of a medicament for decreasing tumor size in a subject with a tumor.

In some embodiments, described herein is a method for enhancing tumor cell removal in a subject with a tumor comprising administering to the subject a binding agent (e.g., an antibody) described herein or a pharmaceutical composition described herein. In some embodiments, provided herein is a binding agent (e.g., an antibody) described herein or a pharmaceutical composition described herein for use in enhancing tumor cell removal in a subject with a tumor. In some embodiments, provided herein is a use of a binding agent (e.g., an antibody) described herein or a pharmaceutical composition described herein in the manufacture of a medicament for enhancing tumor cell removal in a subject with a tumor.

“Enhancing” tumor cell removal includes, yet does not require a 100% enhancement of removal. Any enhancement in the rate of removal is contemplated. Similarly, “modulating” tumor growth refers to reducing the size of the tumor, slowing tumor growth, or inhibiting an increase in the size of an existing tumor. Complete abolition of a tumor is included but not required; any decrease in tumor size or slowing of tumor growth constitutes a beneficial biological effect in a subject. In this regard, tumor cell removal may be enhanced by, for example, at least about 5%, at least about 10% or at least about 20% compared to levels of removal observed in the absence of the method (e.g., in a biologically-matched control subject or specimen that is not exposed to the agent of the method). The effect is detected by, for example, a reduction in tumor size, a decrease or maintenance of the levels of tumor markers, or reduction or maintenance of a tumor cell population. In some embodiments, removal of tumor cells is enhanced by, for example, at least about 30%, at least about 40%, at least about 50%, at least about 60%, at least about 70%, at least about 80%, at least about 90%, or more (about 100%) compared to the removal of tumor cells in the absence of a binding agent (e.g., an anti-LILRB1 antibody, an anti-LILRB2 antibody, or a multispecific antibody or fragment thereof that binds to both LILRB1 and LILRB2) or a pharmaceutical composition of the method.

A method of modulating (e.g., inhibiting, reducing, preventing) tumor growth in a subject also is provided. For example, the method comprises administering to the subject a composition comprising a binding agent (e.g., an anti-LILRB1 antibody, an anti-LILRB2 antibody, or a multispecific antibody or fragment thereof that binds to both LILRB1 and LILRB2) or another composition as disclosed herein in an amount effective to modulate tumor growth in the subject.

In some embodiments, the disease or disorder is a cancer or a tumor.

In some embodiments, the cancer is blood cancer. In some embodiments, the cancer is leukemia. In some embodiments, the cancer is lymphoma.

In some embodiments, the cancer is solid tumor.

In further embodiments, the cancer or tumor expresses HLA-A2. Additionally or

alternatively, the cancer or tumor expresses HLA-G . . . . In further embodiments, the cancer or tumor expresses ANGPTLs. In some embodiments, the subject is a human subject.

HLA-A2 is one of the most frequent HLA class I specificities and thus is extensively studied structurally and functionally (Chen et al. (2012) Immunol Res 182-190). HLA-A2 can bind to LILRB1 and LILRB2 with high affinity.

HLA-G, as a naturally occurring tolerance-inducing molecule, belongs to nonclassical HLA class I family (Rebmann et al. (2014) J Immunol Res 297073). HLA-G can bind to LILRB1 and LILRB2 with high affinity. Its high expression in multiple tumor types, including for example, colorectal, pancreatic, endometrial, lung, breast, ovarian, and gastric cancer, is associated with advanced disease stage, tumor invasiveness, metastatic potential and an unfavorable prognosis (Carosella et al. (2015) Adv Immunol 33-144).

ANGPTLs play a critical role in metabolism. They are ligands for LILRB2 (Zhang et al. (2017) J Leukoc Biol 351-360). ANGPTLs family are also involved in inflammation and tumorigenesis. For example, ANGPTL2 can promotes β-catenin-driven intestinal tumorigenesis (Horiguchi et al. (2022) Oncogene 4028-4041).

As used herein, “tumor” refers to any neoplastic cell growth or proliferation, whether malignant or benign, and to all pre-cancerous and cancerous cells and tissues. The terms “cancer” and “cancerous” refer to or describe the physiological condition in mammals that is typically characterized by unregulated cell growth.

In some embodiments, the tumor or cancer is a solid tumor. In some embodiments, the tumor or cancer is not a solid tumor. In some embodiments, the tumor or cancer is a relapsed tumor or cancer. In some embodiments, the tumor or cancer is a metastatic tumor or cancer. In some embodiments, the tumor or cancer is a primary tumor or cancer. In some embodiments, the tumor or cancer reaches a remission, but can relapse. In some embodiments, the tumor or cancer is unresectable. Additionally or alternatively, the tumor or cancer is resistant to a chemotherapy or other anti-cancer therapy.

Additionally, the binding agents (e.g., antibodies) may be used to alleviate or reduce side effects associated with cancer, for example, bone deterioration, vertebral collapse, and paralysis. In one aspect, the subject suffers from or is at risk of suffering from bone metastases and a binding agent (e.g., an antibody) is administered in an amount to reduce deterioration of surrounding bone. Accordingly, in some aspects, a binding agent provided herein prevents bone deterioration due to bone metastases, wherein tumor cell proliferation is or is not reduced. In some aspects, a binding agent (e.g., an antibody) both prevents bone deterioration due to bone metastases and reduces tumor cell proliferation. In general, the effect on tumor cell proliferation (e.g., inhibition of proliferation or no effect on proliferation) depends on the microenvironment of a particular metastasis. For example, proliferation of metastases located in microenvironments with substantial amounts of type 1 collagen may be inhibited. In contrast, proliferation of metastases located in microenvironments lacking substantial amounts of type 1 collagen may not be inhibited, yet bone deterioration in the vicinity of the metastasis is reduced or prevented.

Also provided is a method of treating a disease or disorder (e.g., a cancer) by administering a binding agent (e.g., an antibody) provided herein, or a pharmaceutical composition as disclosed herein, to a subject in need thereof, alone or in combination with another agent.

A particular administration regimen of a binding agent (e.g., an antibody) or pharmaceutical composition as disclosed herein for a particular subject will depend, in part, upon the agent used, the amount of agent administered, the route of administration, and the cause and extent of any side effects. The amount of the binding agent (e.g., an antibody) administered to a subject (e.g., a mammal, such as a human) should be sufficient to effect the desired response over a reasonable time frame. According, in some embodiments, the amount of a binding agent (e.g., an antibody) or pharmaceutical composition described herein administered to a subject is an effective amount.

Suitable routes of administering a binding agent (e.g., an antibody), such as a multispecific binding agent (e.g., a bispecific antibody) or a composition described herein, are well known in the art, such as intravenous injection (such as intravenous infusion), intratumoral injection, or injection adjacent to a tumor or cancer. Although more than one route can be used to administer an agent (e.g., an antibody), a particular route can provide a more immediate and more effective reaction than another route.

### Combination Therapies

In some embodiments of the methods described herein, a method comprises administering a binding agent (e.g. an anti-LILRB1 antibody, an anti-LILRB2 antibody, or a multispecific antibody that binds to both LILRB1 and LILRB2) described herein in combination with at least one additional therapeutic agent or therapeutic therapy. In some embodiments of the methods described herein, a method comprises administering a binding agent described herein in combination with at least one additional therapeutic agent or therapeutic therapy. Treatment with two or more therapeutic agents often uses agents that work by different mechanisms of action, although this is not required. Combination therapy using agents with different mechanisms of action may result in additive or synergetic effects. Combination therapy may allow for a lower dose of each agent than is used in monotherapy, thereby reducing toxic side effects and/or increasing the therapeutic index of the agent(s). Combination therapy may decrease the likelihood that resistance to an agent will develop.

In some embodiments of the methods described herein, the combination of the binding agent described herein (e.g. an anti-LILRB1 antibody, an anti-LILRB2 antibody, or a multispecific antibody that binds to both LILRB1 and LILRB2) and at least one additional therapeutic agent results in additive or synergistic results. In some embodiments, the combination therapy results in an increase in the therapeutic index of the LILRB1 and/or LILRB2 binding agent. In some embodiments, the combination therapy results in an increase in the therapeutic index of the additional therapeutic agent(s). In some embodiments, the combination therapy results in a decrease in the toxicity and/or side effects of the LILRB1 and/or LILRB2 binding agent. In some embodiments, the combination therapy results in a decrease in the toxicity and/or side effects of the additional therapeutic agent(s). In some embodiments, combination therapy comprises a therapeutic agent that affects the immune response (e.g., enhances or activates the response) and a therapeutic agent that affects (e.g., inhibits or kills) the tumor/cancer cells.

In some embodiments of the methods described herein, a combination treatment comprises one additional therapeutic agent. In some embodiments of the methods described herein, a combination treatment comprises at least one additional therapeutic agent. In some embodiments of the methods described herein, a combination treatment comprises two or more additional therapeutic agents.

The subject of a method described herein can be administered one or more additional therapeutic agents in combination with a binding agent (e.g., an antibody) described herein or fragment thereof or a pharmaceutical composition described herein. An additional agent can be an agent that targets a tumor or a cancer cell. An additional agent can also be an agent that targets an immune cell (e.g., an NK cell or a T cell). In some embodiments, the binding agent or pharmaceutical composition provided herein increases a therapeutic effect of the additional agent by about 10%-90% or about 2-100 folds. In some embodiments, the binding agent or pharmaceutical composition provided herein increases a therapeutic effect of the additional agent by at least 10%. In some embodiments, the binding agent or pharmaceutical composition provided herein increases a therapeutic effect of the additional agent by at least 20%. In some embodiments, the binding agent or pharmaceutical composition provided herein increases a therapeutic effect of the additional agent by at least 30%. In some embodiments, the binding agent or pharmaceutical composition provided herein increases a therapeutic effect of the additional agent by at least 40%. In some embodiments, the binding agent or pharmaceutical composition provided herein increases a therapeutic effect of the additional agent by at least 50%. In some embodiments, the binding agent or pharmaceutical composition provided herein increases a therapeutic effect of the additional agent by at least 60%. In some embodiments, the binding agent or pharmaceutical composition provided herein increases a therapeutic effect of the additional agent by at least 70%. In some embodiments, the binding agent or pharmaceutical composition provided herein increases a therapeutic effect of the additional agent by at least 80%. In some embodiments, the binding agent or pharmaceutical composition provided herein increases a therapeutic effect of the additional agent by at least 90%. In some embodiments, the binding agent or pharmaceutical composition provided herein increases a therapeutic effect of the additional agent by at least 2 fold. In some embodiments, the binding agent or pharmaceutical composition provided herein increases a therapeutic effect of the additional agent by at least 5 fold. In some embodiments, the binding agent or pharmaceutical composition provided herein increases a therapeutic effect of the additional agent by at least 10 fold. In some embodiments, the binding agent or pharmaceutical composition provided herein increases a therapeutic effect of the additional agent by at least 20 fold. In some embodiments, the binding agent or pharmaceutical composition provided herein increases a therapeutic effect of the additional agent by more than 50 fold.

In some embodiments of the methods described herein, the additional therapeutic agent is an immunotherapeutic agent. In some embodiments of the methods described herein the immunotherapeutic agent is a modulator of PD-1 activity. In some embodiments of the methods described herein the immunotherapeutic agent is a modulator of PD-L1 activity. In some embodiments, the additional therapeutic agent is an anti-PD-1 antibody. In some embodiments, the additional therapeutic agent is an anti-PD-L1 antibody. In some embodiments, the anti-PD-1 antibody is Nivolumab. In some embodiments, the anti-PD-L1 antibody is LAE005.

In some embodiments of the methods described herein the immunotherapeutic agent is a modulator of CD3 activity. In some embodiments, the additional therapeutic agent is an anti-CD3 antibody. In some embodiments, the anti-CD3 antibody is OKT3.

In some embodiments of the methods described herein the immunotherapeutic agent is a modulator of CD47 activity. In some embodiments, the additional therapeutic agent is an anti-CD47 antibody. In some embodiments, the anti-CD47 antibody is Hu5F9-G4.

In some embodiments of the methods described herein the immunotherapeutic agent is a modulator of Fc receptors. In some embodiments, the additional therapeutic agent is an antibody contains an Fc domain capable of crosslinking Fc receptors.

In some embodiments, the anti-LILRB1 antibody provided herein can be used with an anti-LILRB2 antibody. In some embodiments, the anti-LILRB1 antibody provided herein can be used with an anti-LILRB2 antibody provided herein. In some embodiments, the anti-LILRB2 antibody provided herein can be used with an anti-LILRB1 antibody. In some embodiments, the anti-LILRB2 antibody provided herein can be used with an anti-LILRB1 antibody provided herein.

### Gene and Cellular Therapies

In some embodiments, the composition for use according to the present disclosure comprises one or more nucleic acids encoding a binding agent provided herein (e.g., an antibody or fragment thereof) or complementary nucleic acids thereto. In a specific embodiment, the nucleic acids are administered to a subject for use in a method provided herein, for example, to prevent, manage, treat and/or ameliorate a disease or disorder (e.g., a cancer, for example a cancer expressing HLA-A2, ANGPTLs, and/or HLA-G) by way of gene and/or cell therapy. Such therapy encompasses that performed by the administration to a subject of an expressed or expressible nucleic acid. In an embodiment, the nucleic acids produce their encoded antibody, and the antibody mediates a prophylactic or therapeutic effect.

Any of the methods for recombinant gene expression (or gene therapy) available in the art can be used.

For general review of the methods of gene therapy, see Goldspiel et al., 1993, Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May 1993, TIBTECH 11 (5): 155-215. Methods commonly known in the art of recombinant DNA technology which can be used are described in Ausubel et al. (eds.), Current Protocols in Molecular Biology, John Wiley & Sons, NY (1993); and Kriegler, Gene Transfer and Expression, A Laboratory Manual, Stockton Press, NY (1990).

In a specific embodiment, a composition comprises nucleic acids encoding an antibody or a fusion protein provided herein, the nucleic acids being part of an expression vector that expresses the antibody or fusion proteins or heavy or light chains thereof in a suitable host. In particular, such nucleic acids have promoters, such as heterologous promoters, operably linked to the antibody coding region, the promoter being inducible or constitutive, and, optionally, tissue-specific and/or tumor/cancer-specific. In another particular embodiment, nucleic acid molecules are used in which the antibody coding sequences and any other desired sequences are flanked by regions that promote homologous recombination at a desired site in the genome, thus providing for intrachromosomal expression of the antibody or fusion protein encoding nucleic acids (Koller and Smithies, 1989, Proc. Natl. Acad. Sci. USA 86:8932-8935; Zijlstra et al., 1989, Nature 342:435-438).

Delivery of the nucleic acids into a subject can be either direct, in which case the subject is directly exposed to the nucleic acid or nucleic acid-carrying vectors, or indirect, in which case, cells are first transformed with the nucleic acids in vitro, then transplanted into the subject. These two approaches are known, respectively, as in vivo or ex vivo gene therapy.

In a specific embodiment, the nucleic acid sequences are directly administered in vivo, where the sequences are expressed to produce the encoded product. This can be accomplished by any of numerous methods known in the art, e.g., by constructing them as part of an appropriate nucleic acid expression vector and administering the vector so that the sequences become intracellular, e.g., by infection using defective or attenuated retroviral or other viral vectors (see U.S. Pat. No. 4,980,286), or by direct injection of naked DNA, or by use of microparticle bombardment (e.g., a gene gun; Biolistic, Dupont), or coating with lipids or cell surface receptors or transfecting agents, encapsulation in liposomes, microparticles, or microcapsules, or by administering them in linkage to a peptide which is known to enter the nucleus, by administering it in linkage to a ligand subject to receptor-mediated endocytosis (see, e.g., Wu and Wu, 1987, J. Biol. Chem. 262:4429-4432) (which can be used to target cell types specifically expressing the receptors), etc. In another embodiment, nucleic acid-ligand complexes can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., WO 92/06180; WO 92/22635; WO 92/20316; WO 93/14188, WO 93/20221). Alternatively, the nucleic acid can be introduced intracellularly and incorporated within host cell DNA for expression, by homologous recombination (Koller and Smithies, 1989, Proc. Natl. Acad. Sci. USA 86:8932-8935; and Zijlstra et al., 1989, Nature 342:435-438).

In a specific embodiment, viral vectors that contains nucleic acid sequences encoding an antibody are used. For example, a retroviral vector can be used (see Miller et al., 1993, Meth. Enzymol. 217:581-599). These retroviral vectors contain the components necessary for the correct packaging of the viral genome and integration into the host cell DNA. The nucleic acid sequences encoding the antibody to be used in gene therapy can be cloned into one or more vectors, which facilitates delivery of the gene into a subject. More detail about retroviral vectors can be found in Boesen et al., 1994, Biotherapy 6:291-302, which describes the use of a retroviral vector to deliver the MDR1 gene to hematopoietic stem cells in order to make the stem cells more resistant to chemotherapy. Other references illustrating the use of retroviral vectors in gene therapy are: Clowes et al., 1994, J. Clin. Invest. 93:644-651; Klein et al., 1994, Blood 83:1467-1473; Salmons and Gunzberg, 1993, Human Gene Therapy 4:129-141; and Grossman and Wilson, 1993, Curr. Opin. in Genetics and Devel. 3:110-114.

Adenoviruses are other viral vectors that can be used in the recombinant production of antibodies. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson, 1993, Current Opinion in Genetics and Development 3:499-503 present a review of adenovirus-based gene therapy. Bout et al., 1994, Human Gene Therapy 5:3-10 demonstrated the use of adenovirus vectors to transfer genes to the respiratory epithelia of rhesus monkeys. Other instances of the use of adenoviruses in gene therapy can be found in Rosenfeld et al., 1991, Science 252:431-434; Rosenfeld et al., 1992, Cell 68:143-155; Mastrangeli et al., 1993, J. Clin. Invest. 91:225-234; PCT Publication WO94/12649; and Wang et al., 1995, Gene Therapy 2:775-783. In a specific embodiment, adenovirus vectors are used.

Adeno-associated virus (AAV) can also be utilized (Walsh et al., 1993, Proc. Soc. Exp. Biol. Med. 204:289-300; and U.S. Pat. No. 5,436,146). In a specific embodiment, AAV vectors are used to express an antibody provided herein. In a specific embodiment, AAV vectors are used to express a bispecific antibody provided herein. In certain embodiments, the AAV comprises a nucleic acid encoding a VH domain. In other embodiments, the AAV comprises a nucleic acid encoding a VL domain. In certain embodiments, the AAV comprises a nucleic acid encoding a VH domain and a VL domain. In some embodiments of the methods provided herein, a subject is administered an AAV comprising a nucleic acid encoding a VH domain and an AAV comprising a nucleic acid encoding a VL domain. In other embodiments, a subject is administered an AAV comprising a nucleic acid encoding a VH domain and a VL domain. In certain embodiments, the VH and VL domains are over-expressed.

In some embodiments, oncolytic viruses may be used in the recombinant production of antibodies provided herein. An oncolytic virus may preferentially infect and kill cancer cells. As the infected cancer cells are destroyed by oncolysis, they may release new infectious virus particles or virions to help destroy the remaining tumor. In a specific embodiment, an oncolytic virus is a virus that when injected into a tumor results in tumor regression. In another specific embodiment, an oncolytic virus is a virus that selectively replicates in and kills cancer cells, and spreads within the tumor. In another specific embodiment, an oncolytic virus is a virus that selectively replicates in and kills cancer cells, and spreads within the tumor without causing any significant damage to normal tissue. In some embodiments, an in vitro or ex vivo assay known to one skilled in the art is used to determine the selectively of a virus to replicate in cancer cells versus non-cancerous cells (e.g., healthy cells). In one embodiment, a virus selectively replicates in cancer cells if a statistically significant increase in the number of virus particles is detected in cancer cells in an in vitro assay or ex vivo assay relative to the number of virus particles detected in non-cancerous cells (e.g., healthy cells) in the same assay after incubation with the virus. In another embodiment, a virus selectively kills cancer cells if a statistically significant amount of the cancer cells are killed in an in vitro or ex vivo assay relative to the amount of non-cancerous cells (e.g., healthy cells) killed in the same assay. In one embodiment, an oncolytic virus naturally preferentially replicates in cancer cells and is non-pathogenic in humans. An oncolytic virus may be non-pathogenic in humans due to elevated sensitivity to innate antiviral signal or dependence on oncogenic signaling pathways. In some embodiments, an oncolytic virus is a parovirus (e.g., an autonomous parvovirus), a myxoma virus, an avian paramyxovirus (e.g., Newcastle disease virus), a reovirus, or Seneca valley virus. In one embodiment, an oncolytic virus is wild-type parvovirus H1 (ParvOryx). In another embodiment, an oncolytic virus is Vesicular stomatitis virus. In another embodiment, an oncolytic virus is an avian paramyxovirus. In some embodiments, an oncolytic virus is a genetically engineered influenza virus, measles virus, poliovirus, vaccinia virus, poxvirus, picornavirus, alphavirus, retrovirus, rhabdovirus, reovirus, adenovirus, herpes simplex virus, or vesicular stomatitis virus. In some embodiments, such viruses are attenuated.

Another approach to gene and cell therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a subject.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcellmediated gene transfer, spheroplast fusion, etc. Numerous techniques are known in the art for the introduction of foreign genes into cells (see, e.g., Loeffler and Behr, 1993, Meth. Enzymol. 217:599-618; Cohen et al., 1993, Meth. Enzymol. 217:618-644; Clin. Pharma. Ther. 29:69-92 (1985)) and can be used in accordance with the methods provided herein, provided that the necessary developmental and physiological functions of the recipient cells are not disrupted. The technique should provide for the stable transfer of the nucleic acid to the cell, so that the nucleic acid is expressible by the cell, such as heritable and expressible by its cell progeny.

The resulting recombinant cells can be delivered to a subject by various methods known in the art. Recombinant blood cells (e.g., hematopoietic stem or progenitor cells) can be administered intravenously. The amount of cells envisioned for use depends on the desired effect, patient state, etc., and can be determined by one skilled in the art.

Cells into which a nucleic acid can be introduced for purposes of gene and/or cell therapy encompass any desired, available cell type, and include but are not limited to epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood, fetal liver, etc.

In a specific embodiment, the cell used for cell and/or gene therapy is autologous to the subject. In other embodiments, the cell used for cell and/or gene therapy is allogeneic to the subject, such as an NK cell.

In an embodiment in which recombinant cells are used in gene and/or therapy, nucleic acid sequences encoding an antibody are introduced into the cells such that they are expressible by the cells or their progeny, and the recombinant cells are then administered in vivo for therapeutic effect. In a specific embodiment, stem or progenitor cells are used. Any stem and/or progenitor cells which can be isolated and maintained in vitro can potentially be used in accordance with this embodiment of the methods provided herein (see e.g., WO 94/08598; Stemple and Anderson, 1992, Cell 7 1:973-985; Rheinwald, 1980, Meth. Cell Bio. 21A: 229; and Pittelkow and Scott, 1986, Mayo Clinic Proc. 61:771).

In a specific embodiment, the nucleic acid to be introduced for purposes of gene and/or therapy comprises an inducible promoter operably linked to the coding region, such that expression of the nucleic acid is controllable by controlling the presence or absence of the appropriate inducer of transcription.

### Diagnostic Uses and Methods of Detection

Labeled binding molecules such as labeled antibodies and derivatives and analogs thereof, which immunospecifically bind to an antigen can be used for diagnostic purposes to detect, diagnose, or monitor a disease.

Antibodies provided herein can be used to assay an antigen level in a biological sample using classical immunohistological methods as described herein or as known to those of skill in the art (e.g., see Jalkanen et al., 1985, J. Cell. Biol. 101:976-985; and Jalkanen et al., 1987, J. Cell. Biol. 105:3087-3096). Other antibody-based methods useful for detecting protein gene expression include immunoassays, such as the enzyme linked immunosorbent assay (ELISA) and the radioimmunoassay (RIA). Suitable antibody assay labels are known in the art and include enzyme labels, such as, glucose oxidase; radioisotopes, such as iodine (1251, 1211), carbon (14C), sulfur (35S), tritium (3H), indium (121 In), and technetium (99Tc); luminescent labels, such as luminol; and fluorescent labels, such as fluorescein and rhodamine, and biotin.

It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of 99Tc. The labeled antibody will then accumulate at the location of cells which contain the specific protein. In vivo tumor imaging is described in S. W. Burchiel et al., “Immunopharmacokinetics of Radiolabeled Antibodies and Their Fragments.” (Chapter 13 in Tumor Imaging: The Radiochemical Detection of Cancer, S. W. Burchiel and B. A. Rhodes, eds., Masson Publishing Inc. (1982)).

Depending on several variables, including the type of label used and the mode of administration, the time interval following the administration for permitting the labeled antibody to concentrate at sites in the subject and for unbound labeled antibody to be cleared to background level is 6 to 48 hours or 6 to 24 hours or 6 to 12 hours. In another embodiment the time interval following administration is 5 to 20 days or 5 to 10 days.

Presence of the labeled molecule can be detected in the subject using methods known in the art for in vivo scanning. These methods depend upon the type of label used. Skilled artisans will be able to determine the appropriate method for detecting a particular label. Methods and devices that may be used in the diagnostic methods provided herein include, but are not limited to, computed tomography (CT), whole body scan such as position emission tomography (PET), magnetic resonance imaging (MRI), and sonography.

In a specific embodiment, the molecule is labeled with a radioisotope and is detected in the patient using a radiation responsive surgical instrument (Thurston et al., U.S. Pat. No. 5,441,050). In another embodiment, the molecule is labeled with a fluorescent compound and is detected in the patient using a fluorescence responsive scanning instrument. In another embodiment, the molecule is labeled with a positron emitting metal and is detected in the patient using positron emission-tomography. In yet another embodiment, the molecule is labeled with a paramagnetic label and is detected in a patient using magnetic resonance imaging (MRI).

### 5.7. Kits

Also provided herein are kits comprising an antibody (e.g. the anti-LILRB1 antibody, the anti-LILRB2 antibody, or the multispecific antibody that bind to both LILRB1 and LILRB2) provided herein, or a composition (e.g., a pharmaceutical composition) thereof, packaged into suitable packaging material. A kit optionally includes a label or packaging insert including a description of the components or instructions for use in vitro, in vivo, or ex vivo, of the components therein.

The term “packaging material” refers to a physical structure housing the components of the kit. The packaging material can maintain the components sterilely, and can be made of material commonly used for such purposes (e.g., paper, corrugated fiber, glass, plastic, foil, ampoules, vials, tubes, etc.).

Kits provided herein can include labels or inserts. Labels or inserts include “printed matter,” e.g., paper or cardboard, separate or affixed to a component, a kit or packing material (e.g., a box), or attached to, for example, an ampoule, tube, or vial containing a kit component. Labels or inserts can additionally include a computer readable medium, such as a disk (e.g., hard disk, card, memory disk), optical disk such as CD- or DVD-ROM/RAM, DVD, MP3, magnetic tape, or an electrical storage media such as RAM and ROM or hybrids of these such as magnetic/optical storage media, FLASH media, or memory type cards. Labels or inserts can include information identifying manufacturer information, lot numbers, manufacturer location, and date.

Kits provided herein can additionally include other components. Each component of the kit can be enclosed within an individual container, and all of the various containers can be within a single package. Kits can also be designed for cold storage. A kit can further be designed to contain antibodies provided herein, or cells that contain nucleic acids encoding the antibodies provided herein. The cells in the kit can be maintained under appropriate storage conditions until ready to use.

Also provided herein are panels of antibodies that immunospecifically bind to a LILRB1 and/or a LILRB2 antigen. In specific embodiments, provided herein are panels of antibodies having different association rate constants, different dissociation rate constants, different affinities for LILRB1 and/or LILRB2 antigen, and/or different specificities for a LILRB1 and/or a LILRB2 antigen. In certain embodiments, provided herein are panels of about 10, preferably about 25, about 50, about 75, about 100, about 125, about 150, about 175, about 200, about 250, about 300, about 350, about 400, about 450, about 500, about 550, about 600, about 650, about 700, about 750, about 800, about 850, about 900, about 950, or about 1000 antibodies or more. Panels of antibodies can be used, for example, in 96 well or 384 well plates, such as for assays such as ELISAs.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described herein.

As used herein, numerical values are often presented in a range format throughout this document. The use of a range format is merely for convenience and brevity and should not be construed as an inflexible limitation on the scope of the invention unless the context clearly indicates otherwise. Accordingly, the use of a range expressly includes all possible subranges, all individual numerical values within that range, and all numerical values or numerical ranges including integers within such ranges and fractions of the values or the integers within ranges unless the context clearly indicates otherwise. This construction applies regardless of the breadth of the range and in all contexts throughout this patent document. Thus, for example, reference to a range of 90-100% includes 91-99%, 92-98%, 93-95%, 91-98%, 91-97%, 91-96%, 91-95%, 91-94%, 91-93%, and so forth. Reference to a range of 90-100% also includes 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%, etc., and so forth.

In addition, reference to a range of 1-3, 3-5, 5-10, 10-20, 20-30, 30-40, 40-50, 50-60, 60-70, 70-80, 80-90, 90-100, 100-110, 110-120, 120-130, 130-140, 140-150, 150-160, 160-170, 170-180, 180-190, 190-200, 200-225, 225-250 includes 1, 2, 3, 4, 5, 6, 7, 8, 9, 10, 11, 12, 13, 14, 15, 16, 17, 18, 19, 20, etc. In a further example, reference to a range of 25-250, 250-500, 500-1,000, 1,000-2,500, 2,500-5,000, 5,000-25,000, 25,000-50,000 includes any numerical value or range within or encompassing such values, e.g., 25, 26, 27, 28, 29 . . . 250, 251, 252, 253, 254 . . . 500, 501, 502, 503, 504 . . . , etc.

As also used herein a series of ranges are disclosed throughout this document. The use of a series of ranges includes combinations of the upper and lower ranges to provide another range. This construction applies regardless of the breadth of the range and in all contexts throughout this patent document. Thus, for example, reference to a series of ranges such as 5-10, 10-20, 20-30, 30-40, 40-50, 50-75, 75-100, 100-150, includes ranges such as 5-20, 5-30, 5-40, 5-50, 5-75, 5-100, 5-150, and 10-30, 10-40, 10-50, 10-75, 10-100, 10-150, and 20-40, 20-50, 20-75, 20-100, 20-150, and so forth.

It is understood that modifications which do not substantially affect the activity of the various embodiments described herein are also provided within the definition of the subject matter described herein. Accordingly, the following examples are intended to illustrate but not limit the present disclosure.

### 6. EMBODIMENTS

The present disclosure includes the following non-limiting embodiments:
Embodiment 1. An antibody or antigen binding fragment thereof that binds LILRB1, wherein the antibody or antigen binding fragment comprises:
(1). a HCDR1, a HCDR2, and a HCDR3 as set forth in a VH comprising the amino acid sequence of SEQ ID NO: 1155 and a LCDR1, a LCDR2, and a LCDR3 as set forth in a VL comprising the amino acid sequence of SEQ ID NO: 1156;
(2). a HCDR1, a HCDR2, and a HCDR3 as set forth in a VH comprising the amino acid sequence of SEQ ID NO: 1157 and a LCDR1, a LCDR2, and a LCDR3 as set forth in a VL comprising the amino acid sequence of SEQ ID NO: 1158;
(3). a HCDR1, a HCDR2, and a HCDR3 as set forth in a VH comprising the amino acid sequence of SEQ ID NO: 1159 and a LCDR1, a LCDR2, and a LCDR3 as set forth in a VL comprising the amino acid sequence of SEQ ID NO: 1160;
(4). a HCDR1, a HCDR2, and a HCDR3 as set forth in a VH comprising the amino acid sequence of SEQ ID NO: 1161 and a LCDR1, a LCDR2, and a LCDR3 as set forth in a VL comprising the amino acid sequence of SEQ ID NO: 1162;
(5). a HCDR1, a HCDR2, and a HCDR3 as set forth in a VH comprising the amino acid sequence of SEQ ID NO: 1163 and a LCDR1, a LCDR2, and a LCDR3 as set forth in a VL comprising the amino acid sequence of SEQ ID NO: 1164; or
(6). a HCDR1, a HCDR2, and a HCDR3 as set forth in a VH comprising the amino acid sequence of SEQ ID NO: 1165 and a LCDR1, a LCDR2, and a LCDR3 as set forth in a VL comprising the amino acid sequence of SEQ ID NO: 1166;
(7). a HCDR1, a HCDR2, and a HCDR3 as set forth in a VH comprising the amino acid sequence of SEQ ID NO: 283 and a LCDR1, a LCDR2, and a LCDR3 as set forth in a VL comprising the amino acid sequence of SEQ ID NO: 284;
(8). a HCDR1, a HCDR2, and a HCDR3 as set forth in a VH comprising the amino acid sequence of SEQ ID NO: 285 and a LCDR1, a LCDR2, and a LCDR3 as set forth in a VL comprising the amino acid sequence of SEQ ID NO: 286;
(9). a HCDR1, a HCDR2, and a HCDR3 as set forth in a VH comprising the amino acid sequence of SEQ ID NO: 287 and a LCDR1, a LCDR2, and a LCDR3 as set forth in a VL comprising the amino acid sequence of SEQ ID NO: 288;
(10). a HCDR1, a HCDR2, and a HCDR3 as set forth in a VH comprising the amino acid sequence of SEQ ID NO: 289 and a LCDR1, a LCDR2, and a LCDR3 as set forth in a VL comprising the amino acid sequence of SEQ ID NO: 290;
(11). a HCDR1, a HCDR2, and a HCDR3 as set forth in a VH comprising the amino acid sequence of SEQ ID NO: 291 and a LCDR1, a LCDR2, and a LCDR3 as set forth in a VL comprising the amino acid sequence of SEQ ID NO: 292;
(12). a HCDR1, a HCDR2, and a HCDR3 as set forth in a VH comprising the amino acid sequence of SEQ ID NO: 293 and a LCDR1, a LCDR2, and a LCDR3 as set forth in a VL comprising the amino acid sequence of SEQ ID NO: 294;
(13). a HCDR1, a HCDR2, and a HCDR3 as set forth in a VH comprising the amino acid sequence of SEQ ID NO: 295 and a LCDR1, a LCDR2, and a LCDR3 as set forth in a VL comprising the amino acid sequence of SEQ ID NO: 296;
(14). a HCDR1, a HCDR2, and a HCDR3 as set forth in a VH comprising the amino acid sequence of SEQ ID NO: 297 and a LCDR1, a LCDR2, and a LCDR3 as set forth in a VL comprising the amino acid sequence of SEQ ID NO: 298;
(15). a HCDR1, a HCDR2, and a HCDR3 as set forth in a VH comprising the amino acid sequence of SEQ ID NO: 299 and a LCDR1, a LCDR2, and a LCDR3 as set forth in a VL comprising the amino acid sequence of SEQ ID NO: 300;
(16). a HCDR1, a HCDR2, and a HCDR3 as set forth in a VH comprising the amino acid sequence of SEQ ID NO: 301 and a LCDR1, a LCDR2, and a LCDR3 as set forth in a VL comprising the amino acid sequence of SEQ ID NO: 302;
(17). a HCDR1, a HCDR2, and a HCDR3 as set forth in a VH comprising the amino acid sequence of SEQ ID NO: 303 and a LCDR1, a LCDR2, and a LCDR3 as set forth in a VL comprising the amino acid sequence of SEQ ID NO: 304;
(18). a HCDR1, a HCDR2, and a HCDR3 as set forth in a VH comprising the amino acid sequence of SEQ ID NO: 305 and a LCDR1, a LCDR2, and a LCDR3 as set forth in a VL comprising the amino acid sequence of SEQ ID NO: 306;
(19). a HCDR1, a HCDR2, and a HCDR3 as set forth in a VH comprising the amino acid sequence of SEQ ID NO: 307 and a LCDR1, a LCDR2, and a LCDR3 as set forth in a VL comprising the amino acid sequence of SEQ ID NO: 308;
(20). a HCDR1, a HCDR2, and a HCDR3 as set forth in a VH comprising the amino acid sequence of SEQ ID NO: 309 and a LCDR1, a LCDR2, and a LCDR3 as set forth in a VL comprising the amino acid sequence of SEQ ID NO: 310;
(21). a HCDR1, a HCDR2, and a HCDR3 as set forth in a VH comprising the amino acid sequence of SEQ ID NO: 311 and a LCDR1, a LCDR2, and a LCDR3 as set forth in a VL comprising the amino acid sequence of SEQ ID NO: 312;
(22). a HCDR1, a HCDR2, and a HCDR3 as set forth in a VH comprising the amino acid sequence of SEQ ID NO: 313 and a LCDR1, a LCDR2, and a LCDR3 as set forth in a VL comprising the amino acid sequence of SEQ ID NO: 314;
(23). a HCDR1, a HCDR2, and a HCDR3 as set forth in a VH comprising the amino acid sequence of SEQ ID NO: 315 and a LCDR1, a LCDR2, and a LCDR3 as set forth in a VL comprising the amino acid sequence of SEQ ID NO: 316;
(24). a HCDR1, a HCDR2, and a HCDR3 as set forth in a VH comprising the amino acid sequence of SEQ ID NO: 317 and a LCDR1, a LCDR2, and a LCDR3 as set forth in a VL comprising the amino acid sequence of SEQ ID NO: 318;
(25). a HCDR1, a HCDR2, and a HCDR3 as set forth in a VH comprising the amino acid sequence of SEQ ID NO: 319 and a LCDR1, a LCDR2, and a LCDR3 as set forth in a VL comprising the amino acid sequence of SEQ ID NO: 320;
(26). a HCDR1, a HCDR2, and a HCDR3 as set forth in a VH comprising the amino acid sequence of SEQ ID NO: 321 and a LCDR1, a LCDR2, and a LCDR3 as set forth in a VL comprising the amino acid sequence of SEQ ID NO: 322;
(27). a HCDR1, a HCDR2, and a HCDR3 as set forth in a VH comprising the amino acid sequence of SEQ ID NO: 323 and a LCDR1, a LCDR2, and a LCDR3 as set forth in a VL comprising the amino acid sequence of SEQ ID NO: 324;
(28). a HCDR1, a HCDR2, and a HCDR3 as set forth in a VH comprising the amino acid sequence of SEQ ID NO: 325 and a LCDR1, a LCDR2, and a LCDR3 as set forth in a VL comprising the amino acid sequence of SEQ ID NO: 326;
(29). a HCDR1, a HCDR2, and a HCDR3 as set forth in a VH comprising the amino acid sequence of SEQ ID NO: 327 and a LCDR1, a LCDR2, and a LCDR3 as set forth in a VL comprising the amino acid sequence of SEQ ID NO: 328;
(30). a HCDR1, a HCDR2, and a HCDR3 as set forth in a VH comprising the amino acid sequence of SEQ ID NO: 329 and a LCDR1, a LCDR2, and a LCDR3 as set forth in a VL comprising the amino acid sequence of SEQ ID NO: 330;
(31). a HCDR1, a HCDR2, and a HCDR3 as set forth in a VH comprising the amino acid sequence of SEQ ID NO: 331 and a LCDR1, a LCDR2, and a LCDR3 as set forth in a VL comprising the amino acid sequence of SEQ ID NO: 332;
(32). a HCDR1, a HCDR2, and a HCDR3 as set forth in a VH comprising the amino acid sequence of SEQ ID NO: 333 and a LCDR1, a LCDR2, and a LCDR3 as set forth in a VL comprising the amino acid sequence of SEQ ID NO: 334;
(33). a HCDR1, a HCDR2, and a HCDR3 as set forth in a VH comprising the amino acid sequence of SEQ ID NO: 335 and a LCDR1, a LCDR2, and a LCDR3 as set forth in a VL comprising the amino acid sequence of SEQ ID NO: 336;
(34). a HCDR1, a HCDR2, and a HCDR3 as set forth in a VH comprising the amino acid sequence of SEQ ID NO: 337 and a LCDR1, a LCDR2, and a LCDR3 as set forth in a VL comprising the amino acid sequence of SEQ ID NO: 338;
(35). a HCDR1, a HCDR2, and a HCDR3 as set forth in a VH comprising the amino acid sequence of SEQ ID NO: 339 and a LCDR1, a LCDR2, and a LCDR3 as set forth in a VL comprising the amino acid sequence of SEQ ID NO: 340;
(36). a HCDR1, a HCDR2, and a HCDR3 as set forth in a VH comprising the amino acid sequence of SEQ ID NO: 341 and a LCDR1, a LCDR2, and a LCDR3 as set forth in a VL comprising the amino acid sequence of SEQ ID NO: 342;
(37). a HCDR1, a HCDR2, and a HCDR3 as set forth in a VH comprising the amino acid sequence of SEQ ID NO: 343 and a LCDR1, a LCDR2, and a LCDR3 as set forth in a VL comprising the amino acid sequence of SEQ ID NO: 344;
(38). a HCDR1, a HCDR2, and a HCDR3 as set forth in a VH comprising the amino acid sequence of SEQ ID NO: 345 and a LCDR1, a LCDR2, and a LCDR3 as set forth in a VL comprising the amino acid sequence of SEQ ID NO: 346;
(39). a HCDR1, a HCDR2, and a HCDR3 as set forth in a VH comprising the amino acid sequence of SEQ ID NO: 347 and a LCDR1, a LCDR2, and a LCDR3 as set forth in a VL comprising the amino acid sequence of SEQ ID NO: 348;
(40). a HCDR1, a HCDR2, and a HCDR3 as set forth in a VH comprising the amino acid sequence of SEQ ID NO: 349 and a LCDR1, a LCDR2, and a LCDR3 as set forth in a VL comprising the amino acid sequence of SEQ ID NO: 350;
(41). a HCDR1, a HCDR2, and a HCDR3 as set forth in a VH comprising the amino acid sequence of SEQ ID NO: 351 and a LCDR1, a LCDR2, and a LCDR3 as set forth in a VL comprising the amino acid sequence of SEQ ID NO: 352;
(42). a HCDR1, a HCDR2, and a HCDR3 as set forth in a VH comprising the amino acid sequence of SEQ ID NO: 353 and a LCDR1, a LCDR2, and a LCDR3 as set forth in a VL comprising the amino acid sequence of SEQ ID NO: 354;
(43). a HCDR1, a HCDR2, and a HCDR3 as set forth in a VH comprising the amino acid sequence of SEQ ID NO: 355 and a LCDR1, a LCDR2, and a LCDR3 as set forth in a VL comprising the amino acid sequence of SEQ ID NO: 356;
(44). a HCDR1, a HCDR2, and a HCDR3 as set forth in a VH comprising the amino acid sequence of SEQ ID NO: 357 and a LCDR1, a LCDR2, and a LCDR3 as set forth in a VL comprising the amino acid sequence of SEQ ID NO: 358;
(45). a HCDR1, a HCDR2, and a HCDR3 as set forth in a VH comprising the amino acid sequence of SEQ ID NO: 359 and a LCDR1, a LCDR2, and a LCDR3 as set forth in a VL comprising the amino acid sequence of SEQ ID NO: 360;
(46). a HCDR1, a HCDR2, and a HCDR3 as set forth in a VH comprising the amino acid sequence of SEQ ID NO: 361 and a LCDR1, a LCDR2, and a LCDR3 as set forth in a VL comprising the amino acid sequence of SEQ ID NO: 362;
(47). a HCDR1, a HCDR2, and a HCDR3 as set forth in a VH comprising the amino acid sequence of SEQ ID NO: 363 and a LCDR1, a LCDR2, and a LCDR3 as set forth in a VL comprising the amino acid sequence of SEQ ID NO: 364;
(48). a HCDR1, a HCDR2, and a HCDR3 as set forth in a VH comprising the amino acid sequence of SEQ ID NO: 365 and a LCDR1, a LCDR2, and a LCDR3 as set forth in a VL comprising the amino acid sequence of SEQ ID NO: 366;
(49). a HCDR1, a HCDR2, and a HCDR3 as set forth in a VH comprising the amino acid sequence of SEQ ID NO: 367 and a LCDR1, a LCDR2, and a LCDR3 as set forth in a VL comprising the amino acid sequence of SEQ ID NO: 368;
(50). a HCDR1, a HCDR2, and a HCDR3 as set forth in a VH comprising the amino acid sequence of SEQ ID NO: 369 and a LCDR1, a LCDR2, and a LCDR3 as set forth in a VL comprising the amino acid sequence of SEQ ID NO: 370;
(51). a HCDR1, a HCDR2, and a HCDR3 as set forth in a VH comprising the amino acid sequence of SEQ ID NO: 371 and a LCDR1, a LCDR2, and a LCDR3 as set forth in a VL comprising the amino acid sequence of SEQ ID NO: 372;
(52). a HCDR1, a HCDR2, and a HCDR3 as set forth in a VH comprising the amino acid sequence of SEQ ID NO: 373 and a LCDR1, a LCDR2, and a LCDR3 as set forth in a VL comprising the amino acid sequence of SEQ ID NO: 374;
(53). a HCDR1, a HCDR2, and a HCDR3 as set forth in a VH comprising the amino acid sequence of SEQ ID NO: 375 and a LCDR1, a LCDR2, and a LCDR3 as set forth in a VL comprising the amino acid sequence of SEQ ID NO: 376;
(54). a HCDR1, a HCDR2, and a HCDR3 as set forth in a VH comprising the amino acid sequence of SEQ ID NO: 377 and a LCDR1, a LCDR2, and a LCDR3 as set forth in a VL comprising the amino acid sequence of SEQ ID NO: 378;
(55). a HCDR1, a HCDR2, and a HCDR3 as set forth in a VH comprising the amino acid sequence of SEQ ID NO: 379 and a LCDR1, a LCDR2, and a LCDR3 as set forth in a VL comprising the amino acid sequence of SEQ ID NO: 380;
(56). a HCDR1, a HCDR2, and a HCDR3 as set forth in a VH comprising the amino acid sequence of SEQ ID NO: 381 and a LCDR1, a LCDR2, and a LCDR3 as set forth in a VL comprising the amino acid sequence of SEQ ID NO: 382;
(57). a HCDR1, a HCDR2, and a HCDR3 as set forth in a VH comprising the amino acid sequence of SEQ ID NO: 383 and a LCDR1, a LCDR2, and a LCDR3 as set forth in a VL comprising the amino acid sequence of SEQ ID NO: 384;
(58). a HCDR1, a HCDR2, and a HCDR3 as set forth in a VH comprising the amino acid sequence of SEQ ID NO: 385 and a LCDR1, a LCDR2, and a LCDR3 as set forth in a VL comprising the amino acid sequence of SEQ ID NO: 386;
(59). a HCDR1, a HCDR2, and a HCDR3 as set forth in a VH comprising the amino acid sequence of SEQ ID NO: 387 and a LCDR1, a LCDR2, and a LCDR3 as set forth in a VL comprising the amino acid sequence of SEQ ID NO: 388;
(60). a HCDR1, a HCDR2, and a HCDR3 as set forth in a VH comprising the amino acid sequence of SEQ ID NO: 389 and a LCDR1, a LCDR2, and a LCDR3 as set forth in a VL comprising the amino acid sequence of SEQ ID NO: 390;
(61). a HCDR1, a HCDR2, and a HCDR3 as set forth in a VH comprising the amino acid sequence of SEQ ID NO: 391 and a LCDR1, a LCDR2, and a LCDR3 as set forth in a VL comprising the amino acid sequence of SEQ ID NO: 392;
(62). a HCDR1, a HCDR2, and a HCDR3 as set forth in a VH comprising the amino acid sequence of SEQ ID NO: 393 and a LCDR1, a LCDR2, and a LCDR3 as set forth in a VL comprising the amino acid sequence of SEQ ID NO: 394;
(63). a HCDR1, a HCDR2, and a HCDR3 as set forth in a VH comprising the amino acid sequence of SEQ ID NO: 395 and a LCDR1, a LCDR2, and a LCDR3 as set forth in a VL comprising the amino acid sequence of SEQ ID NO: 396;
(64). a HCDR1, a HCDR2, and a HCDR3 as set forth in a VH comprising the amino acid sequence of SEQ ID NO: 397 and a LCDR1, a LCDR2, and a LCDR3 as set forth in a VL comprising the amino acid sequence of SEQ ID NO: 398; or
(65). a HCDR1, a HCDR2, and a HCDR3 as set forth in a VH comprising the amino acid sequence of SEQ ID NO: 399 and a LCDR1, a LCDR2, and a LCDR3 as set forth in a VL comprising the amino acid sequence of SEQ ID NO: 400.
Embodiment 2. The antibody or antigen binding fragment of Embodiment 1, wherein:
(1). the HCDR1 comprises the amino acid sequence of SEQ ID NO: 543, the HCDR2 comprises the amino acid sequence of SEQ ID NO: 544, the HCDR3 comprises the amino acid sequence of SEQ ID NO: 1179, the LCDR1 comprises the amino acid sequence of SEQ ID NO: 546, the LCDR2 comprises the amino acid sequence of SEQ ID NO: 1281, and the LCDR3 comprises the amino acid sequence of SEQ ID NO: 548;
(2). the HCDR1 comprises the amino acid sequence of SEQ ID NO: 585, the HCDR2 comprises the amino acid sequence of SEQ ID NO: 586, the HCDR3 comprises the amino acid sequence of SEQ ID NO: 587, the LCDR1 comprises the amino acid sequence of SEQ ID NO: 588, the LCDR2 comprises the amino acid sequence of SEQ ID NO: 589, and the LCDR3 comprises the amino acid sequence of SEQ ID NO: 590;
(3). the HCDR1 comprises the amino acid sequence of SEQ ID NO: 495, the HCDR2 comprises the amino acid sequence of SEQ ID NO: 496, the HCDR3 comprises the amino acid sequence of SEQ ID NO: 497, the LCDR1 comprises the amino acid sequence of SEQ ID NO: 498, the LCDR2 comprises the amino acid sequence of SEQ ID NO: 499, and the LCDR3 comprises the amino acid sequence of SEQ ID NO: 500;
(4). the HCDR1 comprises the amino acid sequence of SEQ ID NO: 501, the HCDR2 comprises the amino acid sequence of SEQ ID NO: 502, the HCDR3 comprises the amino acid sequence of SEQ ID NO: 503, the LCDR1 comprises the amino acid sequence of SEQ ID NO: 504, the LCDR2 comprises the amino acid sequence of SEQ ID NO: 505, and the LCDR3 comprises the amino acid sequence of SEQ ID NO: 506;
(5). the HCDR1 comprises the amino acid sequence of SEQ ID NO: 507, the HCDR2 comprises the amino acid sequence of SEQ ID NO: 508, the HCDR3 comprises the amino acid sequence of SEQ ID NO: 509, the LCDR1 comprises the amino acid sequence of SEQ ID NO: 510, the LCDR2 comprises the amino acid sequence of SEQ ID NO: 511, and the LCDR3 comprises the amino acid sequence of SEQ ID NO: 512;
(6). the HCDR1 comprises the amino acid sequence of SEQ ID NO: 513, the HCDR2 comprises the amino acid sequence of SEQ ID NO: 514, the HCDR3 comprises the amino acid sequence of SEQ ID NO: 515, the LCDR1 comprises the amino acid sequence of SEQ ID NO: 516, the LCDR2 comprises the amino acid sequence of SEQ ID NO: 517, and the LCDR3 comprises the amino acid sequence of SEQ ID NO: 518;
(7). the HCDR1 comprises the amino acid sequence of SEQ ID NO: 519, the HCDR2 comprises the amino acid sequence of SEQ ID NO: 520, the HCDR3 comprises the amino acid sequence of SEQ ID NO: 521, the LCDR1 comprises the amino acid sequence of SEQ ID NO: 522, the LCDR2 comprises the amino acid sequence of SEQ ID NO: 523, and the LCDR3 comprises the amino acid sequence of SEQ ID NO: 524;
(8). the HCDR1 comprises the amino acid sequence of SEQ ID NO: 525, the HCDR2 comprises the amino acid sequence of SEQ ID NO: 526, the HCDR3 comprises the amino acid sequence of SEQ ID NO: 527, the LCDR1 comprises the amino acid sequence of SEQ ID NO: 528, the LCDR2 comprises the amino acid sequence of SEQ ID NO: 529, and the LCDR3 comprises the amino acid sequence of SEQ ID NO: 530;
(9). the HCDR1 comprises the amino acid sequence of SEQ ID NO: 531, the HCDR2 comprises the amino acid sequence of SEQ ID NO: 532, the HCDR3 comprises the amino acid sequence of SEQ ID NO: 533, the LCDR1 comprises the amino acid sequence of SEQ ID NO: 534, the LCDR2 comprises the amino acid sequence of SEQ ID NO: 535, and the LCDR3 comprises the amino acid sequence of SEQ ID NO: 536;
(10). the HCDR1 comprises the amino acid sequence of SEQ ID NO: 537, the HCDR2 comprises the amino acid sequence of SEQ ID NO: 538, the HCDR3 comprises the amino acid sequence of SEQ ID NO: 539, the LCDR1 comprises the amino acid sequence of SEQ ID NO: 540, the LCDR2 comprises the amino acid sequence of SEQ ID NO: 541, and the LCDR3 comprises the amino acid sequence of SEQ ID NO: 542;
(11). the HCDR1 comprises the amino acid sequence of SEQ ID NO: 543, the HCDR2 comprises the amino acid sequence of SEQ ID NO: 544, the HCDR3 comprises the amino acid sequence of SEQ ID NO: 545, the LCDR1 comprises the amino acid sequence of SEQ ID NO: 546, the LCDR2 comprises the amino acid sequence of SEQ ID NO: 547, and the LCDR3 comprises the amino acid sequence of SEQ ID NO: 548;
(12). the HCDR1 comprises the amino acid sequence of SEQ ID NO: 549, the HCDR2 comprises the amino acid sequence of SEQ ID NO: 550, the HCDR3 comprises the amino acid sequence of SEQ ID NO: 551, the LCDR1 comprises the amino acid sequence of SEQ ID NO: 552, the LCDR2 comprises the amino acid sequence of SEQ ID NO: 553, and the LCDR3 comprises the amino acid sequence of SEQ ID NO: 554;
(13). the HCDR1 comprises the amino acid sequence of SEQ ID NO: 555, the HCDR2 comprises the amino acid sequence of SEQ ID NO: 556, the HCDR3 comprises the amino acid sequence of SEQ ID NO: 557, the LCDR1 comprises the amino acid sequence of SEQ ID NO: 558, the LCDR2 comprises the amino acid sequence of SEQ ID NO: 559, and the LCDR3 comprises the amino acid sequence of SEQ ID NO: 560;
(14). the HCDR1 comprises the amino acid sequence of SEQ ID NO: 561, the HCDR2 comprises the amino acid sequence of SEQ ID NO: 562, the HCDR3 comprises the amino acid sequence of SEQ ID NO: 563, the LCDR1 comprises the amino acid sequence of SEQ ID NO: 564, the LCDR2 comprises the amino acid sequence of SEQ ID NO: 565, and the LCDR3 comprises the amino acid sequence of SEQ ID NO: 566;
(15). the HCDR1 comprises the amino acid sequence of SEQ ID NO: 567, the HCDR2 comprises the amino acid sequence of SEQ ID NO: 568, the HCDR3 comprises the amino acid sequence of SEQ ID NO: 569, the LCDR1 comprises the amino acid sequence of SEQ ID NO: 570, the LCDR2 comprises the amino acid sequence of SEQ ID NO: 571, and the LCDR3 comprises the amino acid sequence of SEQ ID NO: 572;
(16). the HCDR1 comprises the amino acid sequence of SEQ ID NO: 573, the HCDR2 comprises the amino acid sequence of SEQ ID NO: 574, the HCDR3 comprises the amino acid sequence of SEQ ID NO: 575, the LCDR1 comprises the amino acid sequence of SEQ ID NO: 576, the LCDR2 comprises the amino acid sequence of SEQ ID NO: 577, and the LCDR3 comprises the amino acid sequence of SEQ ID NO: 578;
(17). the HCDR1 comprises the amino acid sequence of SEQ ID NO: 579, the HCDR2 comprises the amino acid sequence of SEQ ID NO: 580, the HCDR3 comprises the amino acid sequence of SEQ ID NO: 581, the LCDR1 comprises the amino acid sequence of SEQ ID NO: 582, the LCDR2 comprises the amino acid sequence of SEQ ID NO: 583, and the LCDR3 comprises the amino acid sequence of SEQ ID NO: 584;
(18). the HCDR1 comprises the amino acid sequence of SEQ ID NO: 591, the HCDR2 comprises the amino acid sequence of SEQ ID NO: 592, the HCDR3 comprises the amino acid sequence of SEQ ID NO: 593, the LCDR1 comprises the amino acid sequence of SEQ ID NO: 594, the LCDR2 comprises the amino acid sequence of SEQ ID NO: 595, and the LCDR3 comprises the amino acid sequence of SEQ ID NO: 596;
(19). the HCDR1 comprises the amino acid sequence of SEQ ID NO: 597, the HCDR2 comprises the amino acid sequence of SEQ ID NO: 598, the HCDR3 comprises the amino acid sequence of SEQ ID NO: 599, the LCDR1 comprises the amino acid sequence of SEQ ID NO: 600, the LCDR2 comprises the amino acid sequence of SEQ ID NO: 601, and the LCDR3 comprises the amino acid sequence of SEQ ID NO: 602;
(20). the HCDR1 comprises the amino acid sequence of SEQ ID NO: 603, the HCDR2 comprises the amino acid sequence of SEQ ID NO: 604, the HCDR3 comprises the amino acid sequence of SEQ ID NO: 605, the LCDR1 comprises the amino acid sequence of SEQ ID NO: 606, the LCDR2 comprises the amino acid sequence of SEQ ID NO: 607, and the LCDR3 comprises the amino acid sequence of SEQ ID NO: 608;
(21). the HCDR1 comprises the amino acid sequence of SEQ ID NO: 609, the HCDR2 comprises the amino acid sequence of SEQ ID NO: 610, the HCDR3 comprises the amino acid sequence of SEQ ID NO: 611, the LCDR1 comprises the amino acid sequence of SEQ ID NO: 612, the LCDR2 comprises the amino acid sequence of SEQ ID NO: 613, and the LCDR3 comprises the amino acid sequence of SEQ ID NO: 614;
(22). the HCDR1 comprises the amino acid sequence of SEQ ID NO: 615, the HCDR2 comprises the amino acid sequence of SEQ ID NO: 616, the HCDR3 comprises the amino acid sequence of SEQ ID NO: 617, the LCDR1 comprises the amino acid sequence of SEQ ID NO: 618, the LCDR2 comprises the amino acid sequence of SEQ ID NO: 619, and the LCDR3 comprises the amino acid sequence of SEQ ID NO: 620;
(23). the HCDR1 comprises the amino acid sequence of SEQ ID NO: 621, the HCDR2 comprises the amino acid sequence of SEQ ID NO: 622, the HCDR3 comprises the amino acid sequence of SEQ ID NO: 623, the LCDR1 comprises the amino acid sequence of SEQ ID NO: 624, the LCDR2 comprises the amino acid sequence of SEQ ID NO: 625, and the LCDR3 comprises the amino acid sequence of SEQ ID NO: 626;
(24). the HCDR1 comprises the amino acid sequence of SEQ ID NO: 627, the HCDR2 comprises the amino acid sequence of SEQ ID NO: 628, the HCDR3 comprises the amino acid sequence of SEQ ID NO: 629, the LCDR1 comprises the amino acid sequence of SEQ ID NO: 630, the LCDR2 comprises the amino acid sequence of SEQ ID NO: 631, and the LCDR3 comprises the amino acid sequence of SEQ ID NO: 632;
(25). the HCDR1 comprises the amino acid sequence of SEQ ID NO: 633, the HCDR2 comprises the amino acid sequence of SEQ ID NO: 634, the HCDR3 comprises the amino acid sequence of SEQ ID NO: 635, the LCDR1 comprises the amino acid sequence of SEQ ID NO: 636, the LCDR2 comprises the amino acid sequence of SEQ ID NO: 637, and the LCDR3 comprises the amino acid sequence of SEQ ID NO: 638;
(26). the HCDR1 comprises the amino acid sequence of SEQ ID NO: 639, the HCDR2 comprises the amino acid sequence of SEQ ID NO: 640, the HCDR3 comprises the amino acid sequence of SEQ ID NO: 641, the LCDR1 comprises the amino acid sequence of SEQ ID NO: 642, the LCDR2 comprises the amino acid sequence of SEQ ID NO: 643, and the LCDR3 comprises the amino acid sequence of SEQ ID NO: 644;
(27). the HCDR1 comprises the amino acid sequence of SEQ ID NO: 645, the HCDR2 comprises the amino acid sequence of SEQ ID NO: 646, the HCDR3 comprises the amino acid sequence of SEQ ID NO: 647, the LCDR1 comprises the amino acid sequence of SEQ ID NO: 648, the LCDR2 comprises the amino acid sequence of SEQ ID NO: 649, and the LCDR3 comprises the amino acid sequence of SEQ ID NO: 650;
(28). the HCDR1 comprises the amino acid sequence of SEQ ID NO: 651, the HCDR2 comprises the amino acid sequence of SEQ ID NO: 652, the HCDR3 comprises the amino acid sequence of SEQ ID NO: 653, the LCDR1 comprises the amino acid sequence of SEQ ID NO: 654, the LCDR2 comprises the amino acid sequence of SEQ ID NO: 655, and the LCDR3 comprises the amino acid sequence of SEQ ID NO: 656;
(29). the HCDR1 comprises the amino acid sequence of SEQ ID NO: 657, the HCDR2 comprises the amino acid sequence of SEQ ID NO: 658, the HCDR3 comprises the amino acid sequence of SEQ ID NO: 659, the LCDR1 comprises the amino acid sequence of SEQ ID NO: 660, the LCDR2 comprises the amino acid sequence of SEQ ID NO: 661, and the LCDR3 comprises the amino acid sequence of SEQ ID NO: 662;
(30). the HCDR1 comprises the amino acid sequence of SEQ ID NO: 663, the HCDR2 comprises the amino acid sequence of SEQ ID NO: 664, the HCDR3 comprises the amino acid sequence of SEQ ID NO: 665, the LCDR1 comprises the amino acid sequence of SEQ ID NO: 666, the LCDR2 comprises the amino acid sequence of SEQ ID NO: 667, and the LCDR3 comprises the amino acid sequence of SEQ ID NO: 668;
(31). the HCDR1 comprises the amino acid sequence of SEQ ID NO: 669, the HCDR2 comprises the amino acid sequence of SEQ ID NO: 670, the HCDR3 comprises the amino acid sequence of SEQ ID NO: 671, the LCDR1 comprises the amino acid sequence of SEQ ID NO: 672, the LCDR2 comprises the amino acid sequence of SEQ ID NO: 673, and the LCDR3 comprises the amino acid sequence of SEQ ID NO: 674;
(32). the HCDR1 comprises the amino acid sequence of SEQ ID NO: 675, the HCDR2 comprises the amino acid sequence of SEQ ID NO: 676, the HCDR3 comprises the amino acid sequence of SEQ ID NO: 677, the LCDR1 comprises the amino acid sequence of SEQ ID NO: 678, the LCDR2 comprises the amino acid sequence of SEQ ID NO: 679, and the LCDR3 comprises the amino acid sequence of SEQ ID NO: 680;
(33). the HCDR1 comprises the amino acid sequence of SEQ ID NO: 681, the HCDR2 comprises the amino acid sequence of SEQ ID NO: 682, the HCDR3 comprises the amino acid sequence of SEQ ID NO: 683, the LCDR1 comprises the amino acid sequence of SEQ ID NO: 684, the LCDR2 comprises the amino acid sequence of SEQ ID NO: 685, and the LCDR3 comprises the amino acid sequence of SEQ ID NO: 686;
(34). the HCDR1 comprises the amino acid sequence of SEQ ID NO: 687, the HCDR2 comprises the amino acid sequence of SEQ ID NO: 688, the HCDR3 comprises the amino acid sequence of SEQ ID NO: 689, the LCDR1 comprises the amino acid sequence of SEQ ID NO: 690, the LCDR2 comprises the amino acid sequence of SEQ ID NO: 691, and the LCDR3 comprises the amino acid sequence of SEQ ID NO: 692;
(35). the HCDR1 comprises the amino acid sequence of SEQ ID NO: 693, the HCDR2 comprises the amino acid sequence of SEQ ID NO: 694, the HCDR3 comprises the amino acid sequence of SEQ ID NO: 695, the LCDR1 comprises the amino acid sequence of SEQ ID NO: 696, the LCDR2 comprises the amino acid sequence of SEQ ID NO: 697, and the LCDR3 comprises the amino acid sequence of SEQ ID NO: 698;
(36). the HCDR1 comprises the amino acid sequence of SEQ ID NO: 699, the HCDR2 comprises the amino acid sequence of SEQ ID NO: 700, the HCDR3 comprises the amino acid sequence of SEQ ID NO: 701, the LCDR1 comprises the amino acid sequence of SEQ ID NO: 702, the LCDR2 comprises the amino acid sequence of SEQ ID NO: 703, and the LCDR3 comprises the amino acid sequence of SEQ ID NO: 704;
(37). the HCDR1 comprises the amino acid sequence of SEQ ID NO: 705, the HCDR2 comprises the amino acid sequence of SEQ ID NO: 706, the HCDR3 comprises the amino acid sequence of SEQ ID NO: 707, the LCDR1 comprises the amino acid sequence of SEQ ID NO: 708, the LCDR2 comprises the amino acid sequence of SEQ ID NO: 709, and the LCDR3 comprises the amino acid sequence of SEQ ID NO: 710;
(38). the HCDR1 comprises the amino acid sequence of SEQ ID NO: 711, the HCDR2 comprises the amino acid sequence of SEQ ID NO: 712, the HCDR3 comprises the amino acid sequence of SEQ ID NO: 713, the LCDR1 comprises the amino acid sequence of SEQ ID NO: 714, the LCDR2 comprises the amino acid sequence of SEQ ID NO: 715, and the LCDR3 comprises the amino acid sequence of SEQ ID NO: 716;
(39). the HCDR1 comprises the amino acid sequence of SEQ ID NO: 717, the HCDR2 comprises the amino acid sequence of SEQ ID NO: 718, the HCDR3 comprises the amino acid sequence of SEQ ID NO: 719, the LCDR1 comprises the amino acid sequence of SEQ ID NO: 720, the LCDR2 comprises the amino acid sequence of SEQ ID NO: 721, and the LCDR3 comprises the amino acid sequence of SEQ ID NO: 722;
(40). the HCDR1 comprises the amino acid sequence of SEQ ID NO: 723, the HCDR2 comprises the amino acid sequence of SEQ ID NO: 724, the HCDR3 comprises the amino acid sequence of SEQ ID NO: 725, the LCDR1 comprises the amino acid sequence of SEQ ID NO: 726, the LCDR2 comprises the amino acid sequence of SEQ ID NO: 727, and the LCDR3 comprises the amino acid sequence of SEQ ID NO: 728;
(41). the HCDR1 comprises the amino acid sequence of SEQ ID NO: 729, the HCDR2 comprises the amino acid sequence of SEQ ID NO: 730, the HCDR3 comprises the amino acid sequence of SEQ ID NO: 731, the LCDR1 comprises the amino acid sequence of SEQ ID NO: 732, the LCDR2 comprises the amino acid sequence of SEQ ID NO: 733, and the LCDR3 comprises the amino acid sequence of SEQ ID NO: 734;
(42). the HCDR1 comprises the amino acid sequence of SEQ ID NO: 735, the HCDR2 comprises the amino acid sequence of SEQ ID NO: 736, the HCDR3 comprises the amino acid sequence of SEQ ID NO: 737, the LCDR1 comprises the amino acid sequence of SEQ ID NO: 738, the LCDR2 comprises the amino acid sequence of SEQ ID NO: 739, and the LCDR3 comprises the amino acid sequence of SEQ ID NO: 740;
(43). the HCDR1 comprises the amino acid sequence of SEQ ID NO: 741, the HCDR2 comprises the amino acid sequence of SEQ ID NO: 742, the HCDR3 comprises the amino acid sequence of SEQ ID NO: 743, the LCDR1 comprises the amino acid sequence of SEQ ID NO: 744, the LCDR2 comprises the amino acid sequence of SEQ ID NO: 745, and the LCDR3 comprises the amino acid sequence of SEQ ID NO: 746;
(44). the HCDR1 comprises the amino acid sequence of SEQ ID NO: 747, the HCDR2 comprises the amino acid sequence of SEQ ID NO: 748, the HCDR3 comprises the amino acid sequence of SEQ ID NO: 749, the LCDR1 comprises the amino acid sequence of SEQ ID NO: 750, the LCDR2 comprises the amino acid sequence of SEQ ID NO: 751, and the LCDR3 comprises the amino acid sequence of SEQ ID NO: 752;
(45). the HCDR1 comprises the amino acid sequence of SEQ ID NO: 753, the HCDR2 comprises the amino acid sequence of SEQ ID NO: 754, the HCDR3 comprises the amino acid sequence of SEQ ID NO: 755, the LCDR1 comprises the amino acid sequence of SEQ ID NO: 756, the LCDR2 comprises the amino acid sequence of SEQ ID NO: 757, and the LCDR3 comprises the amino acid sequence of SEQ ID NO: 758;
(46). the HCDR1 comprises the amino acid sequence of SEQ ID NO: 759, the HCDR2 comprises the amino acid sequence of SEQ ID NO: 760, the HCDR3 comprises the amino acid sequence of SEQ ID NO: 761, the LCDR1 comprises the amino acid sequence of SEQ ID NO: 762, the LCDR2 comprises the amino acid sequence of SEQ ID NO: 763, and the LCDR3 comprises the amino acid sequence of SEQ ID NO: 764;
(47). the HCDR1 comprises the amino acid sequence of SEQ ID NO: 765, the HCDR2 comprises the amino acid sequence of SEQ ID NO: 766, the HCDR3 comprises the amino acid sequence of SEQ ID NO: 767, the LCDR1 comprises the amino acid sequence of SEQ ID NO: 768, the LCDR2 comprises the amino acid sequence of SEQ ID NO: 769, and the LCDR3 comprises the amino acid sequence of SEQ ID NO: 770;
(48). the HCDR1 comprises the amino acid sequence of SEQ ID NO: 771, the HCDR2 comprises the amino acid sequence of SEQ ID NO: 772, the HCDR3 comprises the amino acid sequence of SEQ ID NO: 773, the LCDR1 comprises the amino acid sequence of SEQ ID NO: 774, the LCDR2 comprises the amino acid sequence of SEQ ID NO: 775, and the LCDR3 comprises the amino acid sequence of SEQ ID NO: 776;
(49). the HCDR1 comprises the amino acid sequence of SEQ ID NO: 777, the HCDR2 comprises the amino acid sequence of SEQ ID NO: 778, the HCDR3 comprises the amino acid sequence of SEQ ID NO: 779, the LCDR1 comprises the amino acid sequence of SEQ ID NO: 780, the LCDR2 comprises the amino acid sequence of SEQ ID NO: 781, and the LCDR3 comprises the amino acid sequence of SEQ ID NO: 782;
(50). the HCDR1 comprises the amino acid sequence of SEQ ID NO: 783, the HCDR2 comprises the amino acid sequence of SEQ ID NO: 784, the HCDR3 comprises the amino acid sequence of SEQ ID NO: 785, the LCDR1 comprises the amino acid sequence of SEQ ID NO: 786, the LCDR2 comprises the amino acid sequence of SEQ ID NO: 787, and the LCDR3 comprises the amino acid sequence of SEQ ID NO: 788;
(51). the HCDR1 comprises the amino acid sequence of SEQ ID NO: 789, the HCDR2 comprises the amino acid sequence of SEQ ID NO: 790, the HCDR3 comprises the amino acid sequence of SEQ ID NO: 791, the LCDR1 comprises the amino acid sequence of SEQ ID NO: 792, the LCDR2 comprises the amino acid sequence of SEQ ID NO: 793, and the LCDR3 comprises the amino acid sequence of SEQ ID NO: 794;
(52). the HCDR1 comprises the amino acid sequence of SEQ ID NO: 795, the HCDR2 comprises the amino acid sequence of SEQ ID NO: 796, the HCDR3 comprises the amino acid sequence of SEQ ID NO: 797, the LCDR1 comprises the amino acid sequence of SEQ ID NO: 798, the LCDR2 comprises the amino acid sequence of SEQ ID NO: 799, and the LCDR3 comprises the amino acid sequence of SEQ ID NO: 800;
(53). the HCDR1 comprises the amino acid sequence of SEQ ID NO: 801, the HCDR2 comprises the amino acid sequence of SEQ ID NO: 802, the HCDR3 comprises the amino acid sequence of SEQ ID NO: 803, the LCDR1 comprises the amino acid sequence of SEQ ID NO: 804, the LCDR2 comprises the amino acid sequence of SEQ ID NO: 805, and the LCDR3 comprises the amino acid sequence of SEQ ID NO: 806;
(54). the HCDR1 comprises the amino acid sequence of SEQ ID NO: 807, the HCDR2 comprises the amino acid sequence of SEQ ID NO: 808, the HCDR3 comprises the amino acid sequence of SEQ ID NO: 809, the LCDR1 comprises the amino acid sequence of SEQ ID NO: 810, the LCDR2 comprises the amino acid sequence of SEQ ID NO: 811, and the LCDR3 comprises the amino acid sequence of SEQ ID NO: 812;
(55). the HCDR1 comprises the amino acid sequence of SEQ ID NO: 813, the HCDR2 comprises the amino acid sequence of SEQ ID NO: 814, the HCDR3 comprises the amino acid sequence of SEQ ID NO: 815, the LCDR1 comprises the amino acid sequence of SEQ ID NO: 816, the LCDR2 comprises the amino acid sequence of SEQ ID NO: 817, and the LCDR3 comprises the amino acid sequence of SEQ ID NO: 818;
(56). the HCDR1 comprises the amino acid sequence of SEQ ID NO: 819, the HCDR2 comprises the amino acid sequence of SEQ ID NO: 820, the HCDR3 comprises the amino acid sequence of SEQ ID NO: 821, the LCDR1 comprises the amino acid sequence of SEQ ID NO: 822, the LCDR2 comprises the amino acid sequence of SEQ ID NO: 823, and the LCDR3 comprises the amino acid sequence of SEQ ID NO: 824;
(57). the HCDR1 comprises the amino acid sequence of SEQ ID NO: 825, the HCDR2 comprises the amino acid sequence of SEQ ID NO: 826, the HCDR3 comprises the amino acid sequence of SEQ ID NO: 827, the LCDR1 comprises the amino acid sequence of SEQ ID NO: 828, the LCDR2 comprises the amino acid sequence of SEQ ID NO: 829, and the LCDR3 comprises the amino acid sequence of SEQ ID NO: 830;
(58). the HCDR1 comprises the amino acid sequence of SEQ ID NO: 831, the HCDR2 comprises the amino acid sequence of SEQ ID NO: 832, the HCDR3 comprises the amino acid sequence of SEQ ID NO: 833, the LCDR1 comprises the amino acid sequence of SEQ ID NO: 834, the LCDR2 comprises the amino acid sequence of SEQ ID NO: 835, and the LCDR3 comprises the amino acid sequence of SEQ ID NO: 836;
(59). the HCDR1 comprises the amino acid sequence of SEQ ID NO: 837, the HCDR2 comprises the amino acid sequence of SEQ ID NO: 838, the HCDR3 comprises the amino acid sequence of SEQ ID NO: 839, the LCDR1 comprises the amino acid sequence of SEQ ID NO: 840, the LCDR2 comprises the amino acid sequence of SEQ ID NO: 841, and the LCDR3 comprises the amino acid sequence of SEQ ID NO: 842; or
(60). the HCDR1 comprises the amino acid sequence of SEQ ID NO: 843, the HCDR2 comprises the amino acid sequence of SEQ ID NO: 844, the HCDR3 comprises the amino acid sequence of SEQ ID NO: 845, the LCDR1 comprises the amino acid sequence of SEQ ID NO: 846, the LCDR2 comprises the amino acid sequence of SEQ ID NO: 847, and the LCDR3 comprises the amino acid sequence of SEQ ID NO: 848.
Embodiment 3. The antibody or antigen binding fragment of Embodiment 1 or 2, comprises:
(1). a VH comprising the amino acid sequence of SEQ ID NO: 1155 and a VL comprising the amino acid sequence of SEQ ID NO: 1156;
(2). a VH comprising the amino acid sequence of SEQ ID NO: 1157 and a VL comprising the amino acid sequence of SEQ ID NO: 1158;
(3). a VH comprising the amino acid sequence of SEQ ID NO: 1159 and a VL comprising the amino acid sequence of SEQ ID NO: 1160;
(4). a VH comprising the amino acid sequence of SEQ ID NO: 1161 and a VL comprising the amino acid sequence of SEQ ID NO: 1162;
(5). a VH comprising the amino acid sequence of SEQ ID NO: 1163 and a VL comprising the amino acid sequence of SEQ ID NO: 1164;
(6). a VH comprising the amino acid sequence of SEQ ID NO: 1165 and a VL comprising the amino acid sequence of SEQ ID NO: 1166.
(7). a VH comprising the amino acid sequence of SEQ ID NO: 283 and a VL comprising the amino acid sequence of SEQ ID NO: 284;
(8). a VH comprising the amino acid sequence of SEQ ID NO: 285 and a VL comprising the amino acid sequence of SEQ ID NO: 286;
(9). a VH comprising the amino acid sequence of SEQ ID NO: 287 and a VL comprising the amino acid sequence of SEQ ID NO: 288;
(10). a VH comprising the amino acid sequence of SEQ ID NO: 289 and a VL comprising the amino acid sequence of SEQ ID NO: 290;
(11). a VH comprising the amino acid sequence of SEQ ID NO: 291 and a VL comprising the amino acid sequence of SEQ ID NO: 292;
(12). a VH comprising the amino acid sequence of SEQ ID NO: 293 and a VL comprising the amino acid sequence of SEQ ID NO: 294;
(13). a VH comprising the amino acid sequence of SEQ ID NO: 295 and a VL comprising the amino acid sequence of SEQ ID NO: 296;
(14). a VH comprising the amino acid sequence of SEQ ID NO: 297 and a VL comprising the amino acid sequence of SEQ ID NO: 298;
(15). a VH comprising the amino acid sequence of SEQ ID NO: 299 and a VL comprising the amino acid sequence of SEQ ID NO: 300;
(16). a VH comprising the amino acid sequence of SEQ ID NO: 301 and a VL comprising the amino acid sequence of SEQ ID NO: 302;
(17). a VH comprising the amino acid sequence of SEQ ID NO: 303 and a VL comprising the amino acid sequence of SEQ ID NO: 304;
(18). a VH comprising the amino acid sequence of SEQ ID NO: 305 and a VL comprising the amino acid sequence of SEQ ID NO: 306;
(19). a VH comprising the amino acid sequence of SEQ ID NO: 307 and a VL comprising the amino acid sequence of SEQ ID NO: 308;
(20). a VH comprising the amino acid sequence of SEQ ID NO: 309 and a VL comprising the amino acid sequence of SEQ ID NO: 310;
(21). a VH comprising the amino acid sequence of SEQ ID NO: 311 and a VL comprising the amino acid sequence of SEQ ID NO: 312;
(22). a VH comprising the amino acid sequence of SEQ ID NO: 313 and a VL comprising the amino acid sequence of SEQ ID NO: 314;
(23). a VH comprising the amino acid sequence of SEQ ID NO: 315 and a VL comprising the amino acid sequence of SEQ ID NO: 316;
(24). a VH comprising the amino acid sequence of SEQ ID NO: 317 and a VL comprising the amino acid sequence of SEQ ID NO: 318;
(25). a VH comprising the amino acid sequence of SEQ ID NO: 319 and a VL comprising the amino acid sequence of SEQ ID NO: 320;
(26). a VH comprising the amino acid sequence of SEQ ID NO: 321 and a VL comprising the amino acid sequence of SEQ ID NO: 322;
(27). a VH comprising the amino acid sequence of SEQ ID NO: 323 and a VL comprising the amino acid sequence of SEQ ID NO: 324;
(28). a VH comprising the amino acid sequence of SEQ ID NO: 325 and a VL comprising the amino acid sequence of SEQ ID NO: 326;
(29). a VH comprising the amino acid sequence of SEQ ID NO: 327 and a VL comprising the amino acid sequence of SEQ ID NO: 328;
(30). a VH comprising the amino acid sequence of SEQ ID NO: 329 and a VL comprising the amino acid sequence of SEQ ID NO: 330;
(31). a VH comprising the amino acid sequence of SEQ ID NO: 331 and a VL comprising the amino acid sequence of SEQ ID NO: 332;
(32). a VH comprising the amino acid sequence of SEQ ID NO: 333 and a VL comprising the amino acid sequence of SEQ ID NO: 334;
(33). a VH comprising the amino acid sequence of SEQ ID NO: 335 and a VL comprising the amino acid sequence of SEQ ID NO: 336;
(34). a VH comprising the amino acid sequence of SEQ ID NO: 337 and a VL comprising the amino acid sequence of SEQ ID NO: 338;
(35). a VH comprising the amino acid sequence of SEQ ID NO: 339 and a VL comprising the amino acid sequence of SEQ ID NO: 340;
(36). a VH comprising the amino acid sequence of SEQ ID NO: 341 and a VL comprising the amino acid sequence of SEQ ID NO: 342;
(37). a VH comprising the amino acid sequence of SEQ ID NO: 343 and a VL comprising the amino acid sequence of SEQ ID NO: 344;
(38). a VH comprising the amino acid sequence of SEQ ID NO: 345 and a VL comprising the amino acid sequence of SEQ ID NO: 346;
(39). a VH comprising the amino acid sequence of SEQ ID NO: 347 and a VL comprising the amino acid sequence of SEQ ID NO: 348;
(40). a VH comprising the amino acid sequence of SEQ ID NO: 349 and a VL comprising the amino acid sequence of SEQ ID NO: 350;
(41). a VH comprising the amino acid sequence of SEQ ID NO: 351 and a VL comprising the amino acid sequence of SEQ ID NO: 352;
(42). a VH comprising the amino acid sequence of SEQ ID NO: 353 and a VL comprising the amino acid sequence of SEQ ID NO: 354;
(43). a VH comprising the amino acid sequence of SEQ ID NO: 355 and a VL comprising the amino acid sequence of SEQ ID NO: 356;
(44). a VH comprising the amino acid sequence of SEQ ID NO: 357 and a VL comprising the amino acid sequence of SEQ ID NO: 358;
(45). a VH comprising the amino acid sequence of SEQ ID NO: 359 and a VL comprising the amino acid sequence of SEQ ID NO: 360;
(46). a VH comprising the amino acid sequence of SEQ ID NO: 361 and a VL comprising the amino acid sequence of SEQ ID NO: 362;
(47). a VH comprising the amino acid sequence of SEQ ID NO: 363 and a VL comprising the amino acid sequence of SEQ ID NO: 364;
(48). a VH comprising the amino acid sequence of SEQ ID NO: 365 and a VL comprising the amino acid sequence of SEQ ID NO: 366;
(49). a VH comprising the amino acid sequence of SEQ ID NO: 367 and a VL comprising the amino acid sequence of SEQ ID NO: 368;
(50). a VH comprising the amino acid sequence of SEQ ID NO: 369 and a VL comprising the amino acid sequence of SEQ ID NO: 370;
(51). a VH comprising the amino acid sequence of SEQ ID NO: 371 and a VL comprising the amino acid sequence of SEQ ID NO: 372;
(52). a VH comprising the amino acid sequence of SEQ ID NO: 373 and a VL comprising the amino acid sequence of SEQ ID NO: 374;
(53). a VH comprising the amino acid sequence of SEQ ID NO: 375 and a VL comprising the amino acid sequence of SEQ ID NO: 376;
(54). a VH comprising the amino acid sequence of SEQ ID NO: 377 and a VL comprising the amino acid sequence of SEQ ID NO: 378;
(55). a VH comprising the amino acid sequence of SEQ ID NO: 379 and a VL comprising the amino acid sequence of SEQ ID NO: 380;
(56). a VH comprising the amino acid sequence of SEQ ID NO: 381 and a VL comprising the amino acid sequence of SEQ ID NO: 382;
(57). a VH comprising the amino acid sequence of SEQ ID NO: 383 and a VL comprising the amino acid sequence of SEQ ID NO: 384;
(58). a VH comprising the amino acid sequence of SEQ ID NO: 385 and a VL comprising the amino acid sequence of SEQ ID NO: 386;
(59). a VH comprising the amino acid sequence of SEQ ID NO: 387 and a VL comprising the amino acid sequence of SEQ ID NO: 388;
(60). a VH comprising the amino acid sequence of SEQ ID NO: 389 and a VL comprising the amino acid sequence of SEQ ID NO: 390;
(61). a VH comprising the amino acid sequence of SEQ ID NO: 391 and a VL comprising the amino acid sequence of SEQ ID NO: 392;
(62). a VH comprising the amino acid sequence of SEQ ID NO: 393 and a VL comprising the amino acid sequence of SEQ ID NO: 394;
(63). a VH comprising the amino acid sequence of SEQ ID NO: 395 and a VL comprising the amino acid sequence of SEQ ID NO: 396;
(64). a VH comprising the amino acid sequence of SEQ ID NO: 397 and a VL comprising the amino acid sequence of SEQ ID NO: 398; or
(65). a VH comprising the amino acid sequence of SEQ ID NO: 399 and a VL comprising the amino acid sequence of SEQ ID NO: 400.
Embodiment 4. An antibody or antigen binding fragment thereof that binds LILRB2, wherein the antibody or antigen binding fragment comprises:
(1). a HCDR1, a HCDR2, and a HCDR3 as set forth in a VH comprising the amino acid sequence of SEQ ID NO: 1167 and a LCDR1, a LCDR2, and a LCDR3 as set forth in a VL comprising the amino acid sequence of SEQ ID NO: 1168;
(2). a HCDR1, a HCDR2, and a HCDR3 as set forth in a VH comprising the amino acid sequence of SEQ ID NO: 1169 and a LCDR1, a LCDR2, and a LCDR3 as set forth in a VL comprising the amino acid sequence of SEQ ID NO: 1170;
(3). a HCDR1, a HCDR2, and a HCDR3 as set forth in a VH comprising the amino acid sequence of SEQ ID NO: 1171 and a LCDR1, a LCDR2, and a LCDR3 as set forth in a VL comprising the amino acid sequence of SEQ ID NO: 1172;
(4). a HCDR1, a HCDR2, and a HCDR3 as set forth in a VH comprising the amino acid sequence of SEQ ID NO: 1173 and a LCDR1, a LCDR2, and a LCDR3 as set forth in a VL comprising the amino acid sequence of SEQ ID NO: 1174;
(5). a HCDR1, a HCDR2, and a HCDR3 as set forth in a VH comprising the amino acid sequence of SEQ ID NO: 1175 and a LCDR1, a LCDR2, and a LCDR3 as set forth in a VL comprising the amino acid sequence of SEQ ID NO: 1176;
(6). a HCDR1, a HCDR2, and a HCDR3 as set forth in a VH comprising the amino acid sequence of SEQ ID NO: 1177 and a LCDR1, a LCDR2, and a LCDR3 as set forth in a VL comprising the amino acid sequence of SEQ ID NO: 1178;
(7). a HCDR1, a HCDR2, and a HCDR3 as set forth in a VH comprising the amino acid sequence of SEQ ID NO: 401 and a LCDR1, a LCDR2, and a LCDR3 as set forth in a VL comprising the amino acid sequence of SEQ ID NO: 402;
(8). a HCDR1, a HCDR2, and a HCDR3 as set forth in a VH comprising the amino acid sequence of SEQ ID NO: 403 and a LCDR1, a LCDR2, and a LCDR3 as set forth in a VL comprising the amino acid sequence of SEQ ID NO: 404;
(9). a HCDR1, a HCDR2, and a HCDR3 as set forth in a VH comprising the amino acid sequence of SEQ ID NO: 405 and a LCDR1, a LCDR2, and a LCDR3 as set forth in a VL comprising the amino acid sequence of SEQ ID NO: 406;
(10). a HCDR1, a HCDR2, and a HCDR3 as set forth in a VH comprising the amino acid sequence of SEQ ID NO: 407 and a LCDR1, a LCDR2, and a LCDR3 as set forth in a VL comprising the amino acid sequence of SEQ ID NO: 408;
(11). a HCDR1, a HCDR2, and a HCDR3 as set forth in a VH comprising the amino acid sequence of SEQ ID NO: 409 and a LCDR1, a LCDR2, and a LCDR3 as set forth in a VL comprising the amino acid sequence of SEQ ID NO: 410;
(12). a HCDR1, a HCDR2, and a HCDR3 as set forth in a VH comprising the amino acid sequence of SEQ ID NO: 411 and a LCDR1, a LCDR2, and a LCDR3 as set forth in a VL comprising the amino acid sequence of SEQ ID NO: 412;
(13). a HCDR1, a HCDR2, and a HCDR3 as set forth in a VH comprising the amino acid sequence of SEQ ID NO: 413 and a LCDR1, a LCDR2, and a LCDR3 as set forth in a VL comprising the amino acid sequence of SEQ ID NO: 414;
(14). a HCDR1, a HCDR2, and a HCDR3 as set forth in a VH comprising the amino acid sequence of SEQ ID NO: 415 and a LCDR1, a LCDR2, and a LCDR3 as set forth in a VL comprising the amino acid sequence of SEQ ID NO: 416;
(15). a HCDR1, a HCDR2, and a HCDR3 as set forth in a VH comprising the amino acid sequence of SEQ ID NO: 417 and a LCDR1, a LCDR2, and a LCDR3 as set forth in a VL comprising the amino acid sequence of SEQ ID NO: 418;
(16). a HCDR1, a HCDR2, and a HCDR3 as set forth in a VH comprising the amino acid sequence of SEQ ID NO: 419 and a LCDR1, a LCDR2, and a LCDR3 as set forth in a VL comprising the amino acid sequence of SEQ ID NO: 420;
(17). a HCDR1, a HCDR2, and a HCDR3 as set forth in a VH comprising the amino acid sequence of SEQ ID NO: 421 and a LCDR1, a LCDR2, and a LCDR3 as set forth in a VL comprising the amino acid sequence of SEQ ID NO: 422;
(18). a HCDR1, a HCDR2, and a HCDR3 as set forth in a VH comprising the amino acid sequence of SEQ ID NO: 423 and a LCDR1, a LCDR2, and a LCDR3 as set forth in a VL comprising the amino acid sequence of SEQ ID NO: 424;
(19). a HCDR1, a HCDR2, and a HCDR3 as set forth in a VH comprising the amino acid sequence of SEQ ID NO: 425 and a LCDR1, a LCDR2, and a LCDR3 as set forth in a VL comprising the amino acid sequence of SEQ ID NO: 426;
(20). a HCDR1, a HCDR2, and a HCDR3 as set forth in a VH comprising the amino acid sequence of SEQ ID NO: 427 and a LCDR1, a LCDR2, and a LCDR3 as set forth in a VL comprising the amino acid sequence of SEQ ID NO: 428;
(21). a HCDR1, a HCDR2, and a HCDR3 as set forth in a VH comprising the amino acid sequence of SEQ ID NO: 429 and a LCDR1, a LCDR2, and a LCDR3 as set forth in a VL comprising the amino acid sequence of SEQ ID NO: 430;
(22). a HCDR1, a HCDR2, and a HCDR3 as set forth in a VH comprising the amino acid sequence of SEQ ID NO: 431 and a LCDR1, a LCDR2, and a LCDR3 as set forth in a VL comprising the amino acid sequence of SEQ ID NO: 432;
(23). a HCDR1, a HCDR2, and a HCDR3 as set forth in a VH comprising the amino acid sequence of SEQ ID NO: 433 and a LCDR1, a LCDR2, and a LCDR3 as set forth in a VL comprising the amino acid sequence of SEQ ID NO: 434;
(24). a HCDR1, a HCDR2, and a HCDR3 as set forth in a VH comprising the amino acid sequence of SEQ ID NO: 435 and a LCDR1, a LCDR2, and a LCDR3 as set forth in a VL comprising the amino acid sequence of SEQ ID NO: 436;
(25). a HCDR1, a HCDR2, and a HCDR3 as set forth in a VH comprising the amino acid sequence of SEQ ID NO: 437 and a LCDR1, a LCDR2, and a LCDR3 as set forth in a VL comprising the amino acid sequence of SEQ ID NO: 438;
(26). a HCDR1, a HCDR2, and a HCDR3 as set forth in a VH comprising the amino acid sequence of SEQ ID NO: 439 and a LCDR1, a LCDR2, and a LCDR3 as set forth in a VL comprising the amino acid sequence of SEQ ID NO: 440;
(27). a HCDR1, a HCDR2, and a HCDR3 as set forth in a VH comprising the amino acid sequence of SEQ ID NO: 441 and a LCDR1, a LCDR2, and a LCDR3 as set forth in a VL comprising the amino acid sequence of SEQ ID NO: 442;
(28). a HCDR1, a HCDR2, and a HCDR3 as set forth in a VH comprising the amino acid sequence of SEQ ID NO: 443 and a LCDR1, a LCDR2, and a LCDR3 as set forth in a VL comprising the amino acid sequence of SEQ ID NO: 444;
(29). a HCDR1, a HCDR2, and a HCDR3 as set forth in a VH comprising the amino acid sequence of SEQ ID NO: 445 and a LCDR1, a LCDR2, and a LCDR3 as set forth in a VL comprising the amino acid sequence of SEQ ID NO: 446;
(30). a HCDR1, a HCDR2, and a HCDR3 as set forth in a VH comprising the amino acid sequence of SEQ ID NO: 447 and a LCDR1, a LCDR2, and a LCDR3 as set forth in a VL comprising the amino acid sequence of SEQ ID NO: 448;
(31). a HCDR1, a HCDR2, and a HCDR3 as set forth in a VH comprising the amino acid sequence of SEQ ID NO: 449 and a LCDR1, a LCDR2, and a LCDR3 as set forth in a VL comprising the amino acid sequence of SEQ ID NO: 450;
(32). a HCDR1, a HCDR2, and a HCDR3 as set forth in a VH comprising the amino acid sequence of SEQ ID NO: 451 and a LCDR1, a LCDR2, and a LCDR3 as set forth in a VL comprising the amino acid sequence of SEQ ID NO: 452;
(33). a HCDR1, a HCDR2, and a HCDR3 as set forth in a VH comprising the amino acid sequence of SEQ ID NO: 453 and a LCDR1, a LCDR2, and a LCDR3 as set forth in a VL comprising the amino acid sequence of SEQ ID NO: 454;
(34). a HCDR1, a HCDR2, and a HCDR3 as set forth in a VH comprising the amino acid sequence of SEQ ID NO: 455 and a LCDR1, a LCDR2, and a LCDR3 as set forth in a VL comprising the amino acid sequence of SEQ ID NO: 456;
(35). a HCDR1, a HCDR2, and a HCDR3 as set forth in a VH comprising the amino acid sequence of SEQ ID NO: 457 and a LCDR1, a LCDR2, and a LCDR3 as set forth in a VL comprising the amino acid sequence of SEQ ID NO: 458;
(36). a HCDR1, a HCDR2, and a HCDR3 as set forth in a VH comprising the amino acid sequence of SEQ ID NO: 459 and a LCDR1, a LCDR2, and a LCDR3 as set forth in a VL comprising the amino acid sequence of SEQ ID NO: 460;
(37). a HCDR1, a HCDR2, and a HCDR3 as set forth in a VH comprising the amino acid sequence of SEQ ID NO: 461 and a LCDR1, a LCDR2, and a LCDR3 as set forth in a VL comprising the amino acid sequence of SEQ ID NO: 462;
(38). a HCDR1, a HCDR2, and a HCDR3 as set forth in a VH comprising the amino acid sequence of SEQ ID NO: 463 and a LCDR1, a LCDR2, and a LCDR3 as set forth in a VL comprising the amino acid sequence of SEQ ID NO: 464;
(39). a HCDR1, a HCDR2, and a HCDR3 as set forth in a VH comprising the amino acid sequence of SEQ ID NO: 465 and a LCDR1, a LCDR2, and a LCDR3 as set forth in a VL comprising the amino acid sequence of SEQ ID NO: 466;
(40). a HCDR1, a HCDR2, and a HCDR3 as set forth in a VH comprising the amino acid sequence of SEQ ID NO: 467 and a LCDR1, a LCDR2, and a LCDR3 as set forth in a VL comprising the amino acid sequence of SEQ ID NO: 468;
(41). a HCDR1, a HCDR2, and a HCDR3 as set forth in a VH comprising the amino acid sequence of SEQ ID NO: 469 and a LCDR1, a LCDR2, and a LCDR3 as set forth in a VL comprising the amino acid sequence of SEQ ID NO: 470;
(42). a HCDR1, a HCDR2, and a HCDR3 as set forth in a VH comprising the amino acid sequence of SEQ ID NO: 471 and a LCDR1, a LCDR2, and a LCDR3 as set forth in a VL comprising the amino acid sequence of SEQ ID NO: 472;
(43). a HCDR1, a HCDR2, and a HCDR3 as set forth in a VH comprising the amino acid sequence of SEQ ID NO: 473 and a LCDR1, a LCDR2, and a LCDR3 as set forth in a VL comprising the amino acid sequence of SEQ ID NO: 474;
(44). a HCDR1, a HCDR2, and a HCDR3 as set forth in a VH comprising the amino acid sequence of SEQ ID NO: 475 and a LCDR1, a LCDR2, and a LCDR3 as set forth in a VL comprising the amino acid sequence of SEQ ID NO: 476;
(45). a HCDR1, a HCDR2, and a HCDR3 as set forth in a VH comprising the amino acid sequence of SEQ ID NO: 477 and a LCDR1, a LCDR2, and a LCDR3 as set forth in a VL comprising the amino acid sequence of SEQ ID NO: 478;
(46). a HCDR1, a HCDR2, and a HCDR3 as set forth in a VH comprising the amino acid sequence of SEQ ID NO: 479 and a LCDR1, a LCDR2, and a LCDR3 as set forth in a VL comprising the amino acid sequence of SEQ ID NO: 480;
(47). a HCDR1, a HCDR2, and a HCDR3 as set forth in a VH comprising the amino acid sequence of SEQ ID NO: 481 and a LCDR1, a LCDR2, and a LCDR3 as set forth in a VL comprising the amino acid sequence of SEQ ID NO: 482;
(48). a HCDR1, a HCDR2, and a HCDR3 as set forth in a VH comprising the amino acid sequence of SEQ ID NO: 483 and a LCDR1, a LCDR2, and a LCDR3 as set forth in a VL comprising the amino acid sequence of SEQ ID NO: 484;
(49). a HCDR1, a HCDR2, and a HCDR3 as set forth in a VH comprising the amino acid sequence of SEQ ID NO: 485 and a LCDR1, a LCDR2, and a LCDR3 as set forth in a VL comprising the amino acid sequence of SEQ ID NO: 486;
(50). a HCDR1, a HCDR2, and a HCDR3 as set forth in a VH comprising the amino acid sequence of SEQ ID NO: 487 and a LCDR1, a LCDR2, and a LCDR3 as set forth in a VL comprising the amino acid sequence of SEQ ID NO: 488;
(51). a HCDR1, a HCDR2, and a HCDR3 as set forth in a VH comprising the amino acid sequence of SEQ ID NO: 489 and a LCDR1, a LCDR2, and a LCDR3 as set forth in a VL comprising the amino acid sequence of SEQ ID NO: 490;
(52). a HCDR1, a HCDR2, and a HCDR3 as set forth in a VH comprising the amino acid sequence of SEQ ID NO: 491 and a LCDR1, a LCDR2, and a LCDR3 as set forth in a VL comprising the amino acid sequence of SEQ ID NO: 492; or
(53). a HCDR1, a HCDR2, and a HCDR3 as set forth in a VH comprising the amino acid sequence of SEQ ID NO: 493 and a LCDR1, a LCDR2, and a LCDR3 as set forth in a VL comprising the amino acid sequence of SEQ ID NO: 494.
Embodiment 5. The antibody or antigen binding fragment of embodiment 4, wherein:
(1). the HCDR1 comprises the amino acid sequence of SEQ ID NO: 855, the HCDR2 comprises the amino acid sequence of SEQ ID NO: 856, the HCDR3 comprises the amino acid sequence of SEQ ID NO: 857, the LCDR1 comprises the amino acid sequence of SEQ ID NO: 858, the LCDR2 comprises the amino acid sequence of SEQ ID NO: 1282, and the LCDR3 comprises the amino acid sequence of SEQ ID NO: 860;
(2). the HCDR1 comprises the amino acid sequence of SEQ ID NO: 987, the HCDR2 comprises the amino acid sequence of SEQ ID NO: 988, the HCDR3 comprises the amino acid sequence of SEQ ID NO: 989, the LCDR1 comprises the amino acid sequence of SEQ ID NO: 990, the LCDR2 comprises the amino acid sequence of SEQ ID NO: 991, and the LCDR3 comprises the amino acid sequence of SEQ ID NO: 992;
(3). the HCDR1 comprises the amino acid sequence of SEQ ID NO: 849, the HCDR2 comprises the amino acid sequence of SEQ ID NO: 850, the HCDR3 comprises the amino acid sequence of SEQ ID NO: 851, the LCDR1 comprises the amino acid sequence of SEQ ID NO: 852, the LCDR2 comprises the amino acid sequence of SEQ ID NO: 853, and the LCDR3 comprises the amino acid sequence of SEQ ID NO: 854;
(4). the HCDR1 comprises the amino acid sequence of SEQ ID NO: 855, the HCDR2 comprises the amino acid sequence of SEQ ID NO: 856, the HCDR3 comprises the amino acid sequence of SEQ ID NO: 857, the LCDR1 comprises the amino acid sequence of SEQ ID NO: 858, the LCDR2 comprises the amino acid sequence of SEQ ID NO: 859, and the LCDR3 comprises the amino acid sequence of SEQ ID NO: 860;
(5). the HCDR1 comprises the amino acid sequence of SEQ ID NO: 861, the HCDR2 comprises the amino acid sequence of SEQ ID NO: 862, the HCDR3 comprises the amino acid sequence of SEQ ID NO: 863, the LCDR1 comprises the amino acid sequence of SEQ ID NO: 864, the LCDR2 comprises the amino acid sequence of SEQ ID NO: 865, and the LCDR3 comprises the amino acid sequence of SEQ ID NO: 866;
(6). the HCDR1 comprises the amino acid sequence of SEQ ID NO: 867, the HCDR2 comprises the amino acid sequence of SEQ ID NO: 868, the HCDR3 comprises the amino acid sequence of SEQ ID NO: 869, the LCDR1 comprises the amino acid sequence of SEQ ID NO: 870, the LCDR2 comprises the amino acid sequence of SEQ ID NO: 871, and the LCDR3 comprises the amino acid sequence of SEQ ID NO: 872;
(7). the HCDR1 comprises the amino acid sequence of SEQ ID NO: 873, the HCDR2 comprises the amino acid sequence of SEQ ID NO: 874, the HCDR3 comprises the amino acid sequence of SEQ ID NO: 875, the LCDR1 comprises the amino acid sequence of SEQ ID NO: 876, the LCDR2 comprises the amino acid sequence of SEQ ID NO: 877, and the LCDR3 comprises the amino acid sequence of SEQ ID NO: 878;
(8). the HCDR1 comprises the amino acid sequence of SEQ ID NO: 879, the HCDR2 comprises the amino acid sequence of SEQ ID NO: 880, the HCDR3 comprises the amino acid sequence of SEQ ID NO: 881, the LCDR1 comprises the amino acid sequence of SEQ ID NO: 882, the LCDR2 comprises the amino acid sequence of SEQ ID NO: 883, and the LCDR3 comprises the amino acid sequence of SEQ ID NO: 884;
(9). the HCDR1 comprises the amino acid sequence of SEQ ID NO: 885, the HCDR2 comprises the amino acid sequence of SEQ ID NO: 886, the HCDR3 comprises the amino acid sequence of SEQ ID NO: 887, the LCDR1 comprises the amino acid sequence of SEQ ID NO: 888, the LCDR2 comprises the amino acid sequence of SEQ ID NO: 889, and the LCDR3 comprises the amino acid sequence of SEQ ID NO: 890;
(10). the HCDR1 comprises the amino acid sequence of SEQ ID NO: 891, the HCDR2 comprises the amino acid sequence of SEQ ID NO: 892, the HCDR3 comprises the amino acid sequence of SEQ ID NO: 893, the LCDR1 comprises the amino acid sequence of SEQ ID NO: 894, the LCDR2 comprises the amino acid sequence of SEQ ID NO: 895, and the LCDR3 comprises the amino acid sequence of SEQ ID NO: 896;
(11). the HCDR1 comprises the amino acid sequence of SEQ ID NO: 897, the HCDR2 comprises the amino acid sequence of SEQ ID NO: 898, the HCDR3 comprises the amino acid sequence of SEQ ID NO: 899, the LCDR1 comprises the amino acid sequence of SEQ ID NO: 900, the LCDR2 comprises the amino acid sequence of SEQ ID NO: 901, and the LCDR3 comprises the amino acid sequence of SEQ ID NO: 902;
(12). the HCDR1 comprises the amino acid sequence of SEQ ID NO: 903, the HCDR2 comprises the amino acid sequence of SEQ ID NO: 904, the HCDR3 comprises the amino acid sequence of SEQ ID NO: 905, the LCDR1 comprises the amino acid sequence of SEQ ID NO: 906, the LCDR2 comprises the amino acid sequence of SEQ ID NO: 907, and the LCDR3 comprises the amino acid sequence of SEQ ID NO: 908;
(13). the HCDR1 comprises the amino acid sequence of SEQ ID NO: 909, the HCDR2 comprises the amino acid sequence of SEQ ID NO: 910, the HCDR3 comprises the amino acid sequence of SEQ ID NO: 911, the LCDR1 comprises the amino acid sequence of SEQ ID NO: 912, the LCDR2 comprises the amino acid sequence of SEQ ID NO: 913, and the LCDR3 comprises the amino acid sequence of SEQ ID NO: 914;
(14). the HCDR1 comprises the amino acid sequence of SEQ ID NO: 915, the HCDR2 comprises the amino acid sequence of SEQ ID NO: 916, the HCDR3 comprises the amino acid sequence of SEQ ID NO: 917, the LCDR1 comprises the amino acid sequence of SEQ ID NO: 918, the LCDR2 comprises the amino acid sequence of SEQ ID NO: 919, and the LCDR3 comprises the amino acid sequence of SEQ ID NO: 920;
(15). the HCDR1 comprises the amino acid sequence of SEQ ID NO: 921, the HCDR2 comprises the amino acid sequence of SEQ ID NO: 922, the HCDR3 comprises the amino acid sequence of SEQ ID NO: 923, the LCDR1 comprises the amino acid sequence of SEQ ID NO: 924, the LCDR2 comprises the amino acid sequence of SEQ ID NO: 925, and the LCDR3 comprises the amino acid sequence of SEQ ID NO: 926;
(16). the HCDR1 comprises the amino acid sequence of SEQ ID NO: 927, the HCDR2 comprises the amino acid sequence of SEQ ID NO: 928, the HCDR3 comprises the amino acid sequence of SEQ ID NO: 929, the LCDR1 comprises the amino acid sequence of SEQ ID NO: 930, the LCDR2 comprises the amino acid sequence of SEQ ID NO: 931, and the LCDR3 comprises the amino acid sequence of SEQ ID NO: 932;
(17). the HCDR1 comprises the amino acid sequence of SEQ ID NO: 933, the HCDR2 comprises the amino acid sequence of SEQ ID NO: 934, the HCDR3 comprises the amino acid sequence of SEQ ID NO: 935, the LCDR1 comprises the amino acid sequence of SEQ ID NO: 936, the LCDR2 comprises the amino acid sequence of SEQ ID NO: 937, and the LCDR3 comprises the amino acid sequence of SEQ ID NO: 938;
(18). the HCDR1 comprises the amino acid sequence of SEQ ID NO: 939, the HCDR2 comprises the amino acid sequence of SEQ ID NO: 940, the HCDR3 comprises the amino acid sequence of SEQ ID NO: 941, the LCDR1 comprises the amino acid sequence of SEQ ID NO: 942, the LCDR2 comprises the amino acid sequence of SEQ ID NO: 943, and the LCDR3 comprises the amino acid sequence of SEQ ID NO: 944;
(19). the HCDR1 comprises the amino acid sequence of SEQ ID NO: 945, the HCDR2 comprises the amino acid sequence of SEQ ID NO: 946, the HCDR3 comprises the amino acid sequence of SEQ ID NO: 947, the LCDR1 comprises the amino acid sequence of SEQ ID NO: 948, the LCDR2 comprises the amino acid sequence of SEQ ID NO: 949, and the LCDR3 comprises the amino acid sequence of SEQ ID NO: 950;
(20). the HCDR1 comprises the amino acid sequence of SEQ ID NO: 951, the HCDR2 comprises the amino acid sequence of SEQ ID NO: 952, the HCDR3 comprises the amino acid sequence of SEQ ID NO: 953, the LCDR1 comprises the amino acid sequence of SEQ ID NO: 954, the LCDR2 comprises the amino acid sequence of SEQ ID NO: 955, and the LCDR3 comprises the amino acid sequence of SEQ ID NO: 956;
(21). the HCDR1 comprises the amino acid sequence of SEQ ID NO: 957, the HCDR2 comprises the amino acid sequence of SEQ ID NO: 958, the HCDR3 comprises the amino acid sequence of SEQ ID NO: 959, the LCDR1 comprises the amino acid sequence of SEQ ID NO: 960, the LCDR2 comprises the amino acid sequence of SEQ ID NO: 961, and the LCDR3 comprises the amino acid sequence of SEQ ID NO: 962;
(22). the HCDR1 comprises the amino acid sequence of SEQ ID NO: 963, the HCDR2 comprises the amino acid sequence of SEQ ID NO: 964, the HCDR3 comprises the amino acid sequence of SEQ ID NO: 965, the LCDR1 comprises the amino acid sequence of SEQ ID NO: 966, the LCDR2 comprises the amino acid sequence of SEQ ID NO: 967, and the LCDR3 comprises the amino acid sequence of SEQ ID NO: 968;
(23). the HCDR1 comprises the amino acid sequence of SEQ ID NO: 969, the HCDR2 comprises the amino acid sequence of SEQ ID NO: 970, the HCDR3 comprises the amino acid sequence of SEQ ID NO: 971, the LCDR1 comprises the amino acid sequence of SEQ ID NO: 972, the LCDR2 comprises the amino acid sequence of SEQ ID NO: 973, and the LCDR3 comprises the amino acid sequence of SEQ ID NO: 974;
(24). the HCDR1 comprises the amino acid sequence of SEQ ID NO: 975, the HCDR2 comprises the amino acid sequence of SEQ ID NO: 976, the HCDR3 comprises the amino acid sequence of SEQ ID NO: 977, the LCDR1 comprises the amino acid sequence of SEQ ID NO: 978, the LCDR2 comprises the amino acid sequence of SEQ ID NO: 979, and the LCDR3 comprises the amino acid sequence of SEQ ID NO: 980;
(25). the HCDR1 comprises the amino acid sequence of SEQ ID NO: 981, the HCDR2 comprises the amino acid sequence of SEQ ID NO: 982, the HCDR3 comprises the amino acid sequence of SEQ ID NO: 983, the LCDR1 comprises the amino acid sequence of SEQ ID NO: 984, the LCDR2 comprises the amino acid sequence of SEQ ID NO: 985, and the LCDR3 comprises the amino acid sequence of SEQ ID NO: 986;
(26). the HCDR1 comprises the amino acid sequence of SEQ ID NO: 993, the HCDR2 comprises the amino acid sequence of SEQ ID NO: 994, the HCDR3 comprises the amino acid sequence of SEQ ID NO: 995, the LCDR1 comprises the amino acid sequence of SEQ ID NO: 996, the LCDR2 comprises the amino acid sequence of SEQ ID NO: 997, and the LCDR3 comprises the amino acid sequence of SEQ ID NO: 998;
(27). the HCDR1 comprises the amino acid sequence of SEQ ID NO: 999, the HCDR2 comprises the amino acid sequence of SEQ ID NO: 1000, the HCDR3 comprises the amino acid sequence of SEQ ID NO: 1001, the LCDR1 comprises the amino acid sequence of SEQ ID NO: 1002, the LCDR2 comprises the amino acid sequence of SEQ ID NO: 1003, and the LCDR3 comprises the amino acid sequence of SEQ ID NO: 1004;
(28). the HCDR1 comprises the amino acid sequence of SEQ ID NO: 1005, the HCDR2 comprises the amino acid sequence of SEQ ID NO: 1006, the HCDR3 comprises the amino acid sequence of SEQ ID NO: 1007, the LCDR1 comprises the amino acid sequence of SEQ ID NO: 1008, the LCDR2 comprises the amino acid sequence of SEQ ID NO: 1009, and the LCDR3 comprises the amino acid sequence of SEQ ID NO: 1010;
(29). the HCDR1 comprises the amino acid sequence of SEQ ID NO: 1011, the HCDR2 comprises the amino acid sequence of SEQ ID NO: 1012, the HCDR3 comprises the amino acid sequence of SEQ ID NO: 1013, the LCDR1 comprises the amino acid sequence of SEQ ID NO: 1014, the LCDR2 comprises the amino acid sequence of SEQ ID NO: 1015, and the LCDR3 comprises the amino acid sequence of SEQ ID NO: 1016;
(30). the HCDR1 comprises the amino acid sequence of SEQ ID NO: 1017, the HCDR2 comprises the amino acid sequence of SEQ ID NO: 1018, the HCDR3 comprises the amino acid sequence of SEQ ID NO: 1019, the LCDR1 comprises the amino acid sequence of SEQ ID NO: 1020, the LCDR2 comprises the amino acid sequence of SEQ ID NO: 1021, and the LCDR3 comprises the amino acid sequence of SEQ ID NO: 1022;
(31). the HCDR1 comprises the amino acid sequence of SEQ ID NO: 1023, the HCDR2 comprises the amino acid sequence of SEQ ID NO: 1024, the HCDR3 comprises the amino acid sequence of SEQ ID NO: 1025, the LCDR1 comprises the amino acid sequence of SEQ ID NO: 1026, the LCDR2 comprises the amino acid sequence of SEQ ID NO: 1027, and the LCDR3 comprises the amino acid sequence of SEQ ID NO: 1028;
(32). the HCDR1 comprises the amino acid sequence of SEQ ID NO: 1029, the HCDR2 comprises the amino acid sequence of SEQ ID NO: 1030, the HCDR3 comprises the amino acid sequence of SEQ ID NO: 1031, the LCDR1 comprises the amino acid sequence of SEQ ID NO: 1032, the LCDR2 comprises the amino acid sequence of SEQ ID NO: 1033, and the LCDR3 comprises the amino acid sequence of SEQ ID NO: 1034;
(33). the HCDR1 comprises the amino acid sequence of SEQ ID NO: 1035, the HCDR2 comprises the amino acid sequence of SEQ ID NO: 1036, the HCDR3 comprises the amino acid sequence of SEQ ID NO: 1037, the LCDR1 comprises the amino acid sequence of SEQ ID NO: 1038, the LCDR2 comprises the amino acid sequence of SEQ ID NO: 1039, and the LCDR3 comprises the amino acid sequence of SEQ ID NO: 1040;
(34). the HCDR1 comprises the amino acid sequence of SEQ ID NO: 1041, the HCDR2 comprises the amino acid sequence of SEQ ID NO: 1042, the HCDR3 comprises the amino acid sequence of SEQ ID NO: 1043, the LCDR1 comprises the amino acid sequence of SEQ ID NO: 1044, the LCDR2 comprises the amino acid sequence of SEQ ID NO: 1045, and the LCDR3 comprises the amino acid sequence of SEQ ID NO: 1046;
(35). the HCDR1 comprises the amino acid sequence of SEQ ID NO: 1047, the HCDR2 comprises the amino acid sequence of SEQ ID NO: 1048, the HCDR3 comprises the amino acid sequence of SEQ ID NO: 1049, the LCDR1 comprises the amino acid sequence of SEQ ID NO: 1050, the LCDR2 comprises the amino acid sequence of SEQ ID NO: 1051, and the LCDR3 comprises the amino acid sequence of SEQ ID NO: 1052;
(36). the HCDR1 comprises the amino acid sequence of SEQ ID NO: 1053, the HCDR2 comprises the amino acid sequence of SEQ ID NO: 1054, the HCDR3 comprises the amino acid sequence of SEQ ID NO: 1055, the LCDR1 comprises the amino acid sequence of SEQ ID NO: 1056, the LCDR2 comprises the amino acid sequence of SEQ ID NO: 1057, and the LCDR3 comprises the amino acid sequence of SEQ ID NO: 1058;
(37). the HCDR1 comprises the amino acid sequence of SEQ ID NO: 1059, the HCDR2 comprises the amino acid sequence of SEQ ID NO: 1060, the HCDR3 comprises the amino acid sequence of SEQ ID NO: 1061, the LCDR1 comprises the amino acid sequence of SEQ ID NO: 1062, the LCDR2 comprises the amino acid sequence of SEQ ID NO: 1063, and the LCDR3 comprises the amino acid sequence of SEQ ID NO: 1064;
(38). the HCDR1 comprises the amino acid sequence of SEQ ID NO: 1065, the HCDR2 comprises the amino acid sequence of SEQ ID NO: 1066, the HCDR3 comprises the amino acid sequence of SEQ ID NO: 1067, the LCDR1 comprises the amino acid sequence of SEQ ID NO: 1068, the LCDR2 comprises the amino acid sequence of SEQ ID NO: 1069, and the LCDR3 comprises the amino acid sequence of SEQ ID NO: 1070;
(39). the HCDR1 comprises the amino acid sequence of SEQ ID NO: 1071, the HCDR2 comprises the amino acid sequence of SEQ ID NO: 1072, the HCDR3 comprises the amino acid sequence of SEQ ID NO: 1073, the LCDR1 comprises the amino acid sequence of SEQ ID NO: 1074, the LCDR2 comprises the amino acid sequence of SEQ ID NO: 1075, and the LCDR3 comprises the amino acid sequence of SEQ ID NO: 1076;
(40). the HCDR1 comprises the amino acid sequence of SEQ ID NO: 1077, the HCDR2 comprises the amino acid sequence of SEQ ID NO: 1078, the HCDR3 comprises the amino acid sequence of SEQ ID NO: 1079, the LCDR1 comprises the amino acid sequence of SEQ ID NO: 1080, the LCDR2 comprises the amino acid sequence of SEQ ID NO: 1081, and the LCDR3 comprises the amino acid sequence of SEQ ID NO: 1082;
(41). the HCDR1 comprises the amino acid sequence of SEQ ID NO: 1083, the HCDR2 comprises the amino acid sequence of SEQ ID NO: 1084, the HCDR3 comprises the amino acid sequence of SEQ ID NO: 1085, the LCDR1 comprises the amino acid sequence of SEQ ID NO: 1086, the LCDR2 comprises the amino acid sequence of SEQ ID NO: 1087, and the LCDR3 comprises the amino acid sequence of SEQ ID NO: 1088;
(42). the HCDR1 comprises the amino acid sequence of SEQ ID NO: 1089, the HCDR2 comprises the amino acid sequence of SEQ ID NO: 1090, the HCDR3 comprises the amino acid sequence of SEQ ID NO: 1091, the LCDR1 comprises the amino acid sequence of SEQ ID NO: 1092, the LCDR2 comprises the amino acid sequence of SEQ ID NO: 1093, and the LCDR3 comprises the amino acid sequence of SEQ ID NO: 1094;
(43). the HCDR1 comprises the amino acid sequence of SEQ ID NO: 1095, the HCDR2 comprises the amino acid sequence of SEQ ID NO: 1096, the HCDR3 comprises the amino acid sequence of SEQ ID NO: 1097, the LCDR1 comprises the amino acid sequence of SEQ ID NO: 1098, the LCDR2 comprises the amino acid sequence of SEQ ID NO: 1099, and the LCDR3 comprises the amino acid sequence of SEQ ID NO: 1100;
(44). the HCDR1 comprises the amino acid sequence of SEQ ID NO: 1101, the HCDR2 comprises the amino acid sequence of SEQ ID NO: 1102, the HCDR3 comprises the amino acid sequence of SEQ ID NO: 1103, the LCDR1 comprises the amino acid sequence of SEQ ID NO: 1104, the LCDR2 comprises the amino acid sequence of SEQ ID NO: 1105, and the LCDR3 comprises the amino acid sequence of SEQ ID NO: 1106;
(45). the HCDR1 comprises the amino acid sequence of SEQ ID NO: 1107, the HCDR2 comprises the amino acid sequence of SEQ ID NO: 1108, the HCDR3 comprises the amino acid sequence of SEQ ID NO: 1109, the LCDR1 comprises the amino acid sequence of SEQ ID NO: 1110, the LCDR2 comprises the amino acid sequence of SEQ ID NO: 1111, and the LCDR3 comprises the amino acid sequence of SEQ ID NO: 1112;
(46). the HCDR1 comprises the amino acid sequence of SEQ ID NO: 1113, the HCDR2 comprises the amino acid sequence of SEQ ID NO: 1114, the HCDR3 comprises the amino acid sequence of SEQ ID NO: 1115, the LCDR1 comprises the amino acid sequence of SEQ ID NO: 1116, the LCDR2 comprises the amino acid sequence of SEQ ID NO: 1117, and the LCDR3 comprises the amino acid sequence of SEQ ID NO: 1118;
(47). the HCDR1 comprises the amino acid sequence of SEQ ID NO: 1119, the HCDR2 comprises the amino acid sequence of SEQ ID NO: 1120, the HCDR3 comprises the amino acid sequence of SEQ ID NO: 1121, the LCDR1 comprises the amino acid sequence of SEQ ID NO: 1122, the LCDR2 comprises the amino acid sequence of SEQ ID NO: 1123, and the LCDR3 comprises the amino acid sequence of SEQ ID NO: 1124; or
(48). the HCDR1 comprises the amino acid sequence of SEQ ID NO: 1125, the HCDR2 comprises the amino acid sequence of SEQ ID NO: 1126, the HCDR3 comprises the amino acid sequence of SEQ ID NO: 1127, the LCDR1 comprises the amino acid sequence of SEQ ID NO: 1128, the LCDR2 comprises the amino acid sequence of SEQ ID NO: 1129, and the LCDR3 comprises the amino acid sequence of SEQ ID NO: 1130.
Embodiment 6. The antibody or antigen binding fragment of embodiment 4 or 5, comprises:
(1). a VH comprising the amino acid sequence of SEQ ID NO: 1167 and a VL comprising the amino acid sequence of SEQ ID NO: 1168;
(2). a VH comprising the amino acid sequence of SEQ ID NO: 1169 and a VL comprising the amino acid sequence of SEQ ID NO: 1170;
(3). a VH comprising the amino acid sequence of SEQ ID NO: 1171 and a VL comprising the amino acid sequence of SEQ ID NO: 1172;
(4). a VH comprising the amino acid sequence of SEQ ID NO: 1173 and a VL comprising the amino acid sequence of SEQ ID NO: 1174;
(5). a VH comprising the amino acid sequence of SEQ ID NO: 1175 and a VL comprising the amino acid sequence of SEQ ID NO: 1176;
(6). a VH comprising the amino acid sequence of SEQ ID NO: 1177 and a VL comprising the amino acid sequence of SEQ ID NO: 1178.
(7). a VH comprising the amino acid sequence of SEQ ID NO: 401 and a VL comprising the amino acid sequence of SEQ ID NO: 402;
(8). a VH comprising the amino acid sequence of SEQ ID NO: 403 and a VL comprising the amino acid sequence of SEQ ID NO: 404;
(9). a VH comprising the amino acid sequence of SEQ ID NO: 405 and a VL comprising the amino acid sequence of SEQ ID NO: 406;
(10). a VH comprising the amino acid sequence of SEQ ID NO: 407 and a VL comprising the amino acid sequence of SEQ ID NO: 408;
(11). a VH comprising the amino acid sequence of SEQ ID NO: 409 and a VL comprising the amino acid sequence of SEQ ID NO: 410;
(12). a VH comprising the amino acid sequence of SEQ ID NO: 411 and a VL comprising the amino acid sequence of SEQ ID NO: 412;
(13). a VH comprising the amino acid sequence of SEQ ID NO: 413 and a VL comprising the amino acid sequence of SEQ ID NO: 414;
(14). a VH comprising the amino acid sequence of SEQ ID NO: 415 and a VL comprising the amino acid sequence of SEQ ID NO: 416;
(15). a VH comprising the amino acid sequence of SEQ ID NO: 417 and a VL comprising the amino acid sequence of SEQ ID NO: 418;
(16). a VH comprising the amino acid sequence of SEQ ID NO: 419 and a VL comprising the amino acid sequence of SEQ ID NO: 420;
(17). a VH comprising the amino acid sequence of SEQ ID NO: 421 and a VL comprising the amino acid sequence of SEQ ID NO: 422;
(18). a VH comprising the amino acid sequence of SEQ ID NO: 423 and a VL comprising the amino acid sequence of SEQ ID NO: 424;
(19). a VH comprising the amino acid sequence of SEQ ID NO: 425 and a VL comprising the amino acid sequence of SEQ ID NO: 426;
(20). a VH comprising the amino acid sequence of SEQ ID NO: 427 and a VL comprising the amino acid sequence of SEQ ID NO: 428;
(21). a VH comprising the amino acid sequence of SEQ ID NO: 429 and a VL comprising the amino acid sequence of SEQ ID NO: 430;
(22). a VH comprising the amino acid sequence of SEQ ID NO: 431 and a VL comprising the amino acid sequence of SEQ ID NO: 432;
(23). a VH comprising the amino acid sequence of SEQ ID NO: 433 and a VL comprising the amino acid sequence of SEQ ID NO: 434;
(24). a VH comprising the amino acid sequence of SEQ ID NO: 435 and a VL comprising the amino acid sequence of SEQ ID NO: 436;
(25). a VH comprising the amino acid sequence of SEQ ID NO: 437 and a VL comprising the amino acid sequence of SEQ ID NO: 438;
(26). a VH comprising the amino acid sequence of SEQ ID NO: 439 and a VL comprising the amino acid sequence of SEQ ID NO: 440;
(27). a VH comprising the amino acid sequence of SEQ ID NO: 441 and a VL comprising the amino acid sequence of SEQ ID NO: 442;
(28). a VH comprising the amino acid sequence of SEQ ID NO: 443 and a VL comprising the amino acid sequence of SEQ ID NO: 444;
(29). a VH comprising the amino acid sequence of SEQ ID NO: 445 and a VL comprising the amino acid sequence of SEQ ID NO: 446;
(30). a VH comprising the amino acid sequence of SEQ ID NO: 447 and a VL comprising the amino acid sequence of SEQ ID NO: 448;
(31). a VH comprising the amino acid sequence of SEQ ID NO: 449 and a VL comprising the amino acid sequence of SEQ ID NO: 450;
(32). a VH comprising the amino acid sequence of SEQ ID NO: 451 and a VL comprising the amino acid sequence of SEQ ID NO: 452;
(33). a VH comprising the amino acid sequence of SEQ ID NO: 453 and a VL comprising the amino acid sequence of SEQ ID NO: 454;
(34). a VH comprising the amino acid sequence of SEQ ID NO: 455 and a VL comprising the amino acid sequence of SEQ ID NO: 456;
(35). a VH comprising the amino acid sequence of SEQ ID NO: 457 and a VL comprising the amino acid sequence of SEQ ID NO: 458;
(36). a VH comprising the amino acid sequence of SEQ ID NO: 459 and a VL comprising the amino acid sequence of SEQ ID NO: 460;
(37). a VH comprising the amino acid sequence of SEQ ID NO: 461 and a VL comprising the amino acid sequence of SEQ ID NO: 462;
(38). a VH comprising the amino acid sequence of SEQ ID NO: 463 and a VL comprising the amino acid sequence of SEQ ID NO: 464;
(39). a VH comprising the amino acid sequence of SEQ ID NO: 465 and a VL comprising the amino acid sequence of SEQ ID NO: 466;
(40). a VH comprising the amino acid sequence of SEQ ID NO: 467 and a VL comprising the amino acid sequence of SEQ ID NO: 468;
(41). a VH comprising the amino acid sequence of SEQ ID NO: 469 and a VL comprising the amino acid sequence of SEQ ID NO: 470;
(42). a VH comprising the amino acid sequence of SEQ ID NO: 471 and a VL comprising the amino acid sequence of SEQ ID NO: 472;
(43). a VH comprising the amino acid sequence of SEQ ID NO: 473 and a VL comprising the amino acid sequence of SEQ ID NO: 474;
(44). a VH comprising the amino acid sequence of SEQ ID NO: 475 and a VL comprising the amino acid sequence of SEQ ID NO: 476;
(45). a VH comprising the amino acid sequence of SEQ ID NO: 477 and a VL comprising the amino acid sequence of SEQ ID NO: 478;
(46). a VH comprising the amino acid sequence of SEQ ID NO: 479 and a VL comprising the amino acid sequence of SEQ ID NO: 480;
(47). a VH comprising the amino acid sequence of SEQ ID NO: 481 and a VL comprising the amino acid sequence of SEQ ID NO: 482;
(48). a VH comprising the amino acid sequence of SEQ ID NO: 483 and a VL comprising the amino acid sequence of SEQ ID NO: 484;
(49). a VH comprising the amino acid sequence of SEQ ID NO: 485 and a VL comprising the amino acid sequence of SEQ ID NO: 486;
(50). a VH comprising the amino acid sequence of SEQ ID NO: 487 and a VL comprising the amino acid sequence of SEQ ID NO: 488;
(51). a VH comprising the amino acid sequence of SEQ ID NO: 489 and a VL comprising the amino acid sequence of SEQ ID NO: 490;
(52). a VH comprising the amino acid sequence of SEQ ID NO: 491 and a VL comprising the amino acid sequence of SEQ ID NO: 492; or
(53). a VH comprising the amino acid sequence of SEQ ID NO: 493 and a VL comprising the amino acid sequence of SEQ ID NO: 494.
Embodiment 7. A multispecific antibody or fragment thereof, comprising a first binding domain that binds to LILRB1 and a second binding domain that binds to LILRB2, wherein the first binding domain comprises an antibody or antigen binding fragment thereof according to embodiments 1 to 3, and wherein optionally the second binding domain comprises an antibody or antigen binding fragment thereof according to embodiments 4 to 6.
Embodiment 8. A multispecific antibody or fragment thereof, comprising a first binding domain that binds to LILRB1 and a second binding domain that binds to LILRB2, wherein the second binding domain comprises an antibody or antigen binding fragment thereof according to embodiments 4 to 6, and wherein optionally the first binding domain comprises an antibody or antigen binding fragment thereof according to embodiments 1 to 3.
Embodiment 9. The multispecific antibody or fragment thereof of embodiment 7 or 8, wherein the multispecific antibody or fragment thereof has an antibody format of immunoglobulin-single chain antibody fragment (IgG-ScFv).
Embodiment 10. The multispecific antibody or fragment thereof of embodiment 7 or 8, wherein the multispecific antibody or fragment thereof has an antibody format of dual-variable-domain-immunoglobulin (DVD-Ig).
Embodiment 11. The multispecific antibody or fragment thereof of embodiment 7 or 8, wherein the first binding domain comprises any one or more of (1)-(6):
(1). a HCDR1, a HCDR2, and a HCDR3 as set forth in a VH comprising the amino acid sequence of SEQ ID NO: 1155 and a LCDR1, a LCDR2, and a LCDR3 as set forth in a VL comprising the amino acid sequence of SEQ ID NO: 1156;
(2). a HCDR1, a HCDR2, and a HCDR3 as set forth in a VH comprising the amino acid sequence of SEQ ID NO: 1157 and a LCDR1, a LCDR2, and a LCDR3 as set forth in a VL comprising the amino acid sequence of SEQ ID NO: 1158;
(3). a HCDR1, a HCDR2, and a HCDR3 as set forth in a VH comprising the amino acid sequence of SEQ ID NO: 1159 and a LCDR1, a LCDR2, and a LCDR3 as set forth in a VL comprising the amino acid sequence of SEQ ID NO: 1160;
(4). a HCDR1, a HCDR2, and a HCDR3 as set forth in a VH comprising the amino acid sequence of SEQ ID NO: 1161 and a LCDR1, a LCDR2, and a LCDR3 as set forth in a VL comprising the amino acid sequence of SEQ ID NO: 1162;
(5). a HCDR1, a HCDR2, and a HCDR3 as set forth in a VH comprising the amino acid sequence of SEQ ID NO: 1163 and a LCDR1, a LCDR2, and a LCDR3 as set forth in a VL comprising the amino acid sequence of SEQ ID NO: 1164; or
(6). a HCDR1, a HCDR2, and a HCDR3 as set forth in a VH comprising the amino acid sequence of SEQ ID NO: 1165 and a LCDR1, a LCDR2, and a LCDR3 as set forth in a VL comprising the amino acid sequence of SEQ ID NO: 1166; and wherein the second binding domain comprises any one or more of (1)-(6):
(1). a HCDR1, a HCDR2, and a HCDR3 as set forth in a VH comprising the amino acid sequence of SEQ ID NO: 1167 and a LCDR1, a LCDR2, and a LCDR3 as set forth in a VL comprising the amino acid sequence of SEQ ID NO: 1168;
(2). a HCDR1, a HCDR2, and a HCDR3 as set forth in a VH comprising the amino acid sequence of SEQ ID NO: 1169 and a LCDR1, a LCDR2, and a LCDR3 as set forth in a VL comprising the amino acid sequence of SEQ ID NO: 1170;
(3). a HCDR1, a HCDR2, and a HCDR3 as set forth in a VH comprising the amino acid sequence of SEQ ID NO: 1171 and a LCDR1, a LCDR2, and a LCDR3 as set forth in a VL comprising the amino acid sequence of SEQ ID NO: 1172;
(4). a HCDR1, a HCDR2, and a HCDR3 as set forth in a VH comprising the amino acid sequence of SEQ ID NO: 1173 and a LCDR1, a LCDR2, and a LCDR3 as set forth in a VL comprising the amino acid sequence of SEQ ID NO: 1174;
(5). a HCDR1, a HCDR2, and a HCDR3 as set forth in a VH comprising the amino acid sequence of SEQ ID NO: 1175 and a LCDR1, a LCDR2, and a LCDR3 as set forth in a VL comprising the amino acid sequence of SEQ ID NO: 1176; or
(6). a HCDR1, a HCDR2, and a HCDR3 as set forth in a VH comprising the amino acid sequence of SEQ ID NO: 1177 and a LCDR1, a LCDR2, and a LCDR3 as set forth in a VL comprising the amino acid sequence of SEQ ID NO: 1178.
Embodiment 12. The multispecific antibody or fragment thereof of embodiment 11, wherein the multispecific antibody or fragment thereof comprises:

wherein the first binding domain comprises any one or more of (1)-(6):

(1). a VH comprising the amino acid sequence of SEQ ID NO: 1155 and a VL comprising the amino acid sequence of SEQ ID NO: 1156;
(2). a VH comprising the amino acid sequence of SEQ ID NO: 1157 and a VL comprising the amino acid sequence of SEQ ID NO: 1158;
(3). a VH comprising the amino acid sequence of SEQ ID NO: 1159 and a VL comprising the amino acid sequence of SEQ ID NO: 1160;
(4). a VH comprising the amino acid sequence of SEQ ID NO: 1161 and a VL comprising the amino acid sequence of SEQ ID NO: 1162;
(5). a VH comprising the amino acid sequence of SEQ ID NO: 1163 and a VL comprising the amino acid sequence of SEQ ID NO: 1164; or
(6) a VH comprising the amino acid sequence of SEQ ID NO: 1165 and a VL comprising the amino acid sequence of SEQ ID NO: 1166; and wherein the second binding domain comprises any one or more of (1)-(6):
(1). a VH comprising the amino acid sequence of SEQ ID NO: 1167 and a VL comprising the amino acid sequence of SEQ ID NO: 1168;
(2). a VH comprising the amino acid sequence of SEQ ID NO: 1169 and a VL comprising the amino acid sequence of SEQ ID NO: 1170;
(3). a VH comprising the amino acid sequence of SEQ ID NO: 1171 and a VL comprising the amino acid sequence of SEQ ID NO: 1172;
(4). a VH comprising the amino acid sequence of SEQ ID NO: 1173 and a VL comprising the amino acid sequence of SEQ ID NO: 1174;
(5). a VH comprising the amino acid sequence of SEQ ID NO: 1175 and a VL comprising the amino acid sequence of SEQ ID NO: 1176; or
(6). a VH comprising the amino acid sequence of SEQ ID NO: 1177 and a VL comprising the amino acid sequence of SEQ ID NO: 1178.
Embodiment 13. The multispecific antibody or fragment thereof of embodiment 11 or 12, wherein the multispecific antibody or fragment thereof comprises:
(1). a first polypeptide comprising the amino acid sequence of SEQ ID NO: 1180, and a second polypeptide comprising the amino acid sequence of SEQ ID NO: 1181;
(2). a first polypeptide comprising the amino acid sequence of SEQ ID NO: 1182, and a second polypeptide comprising the amino acid sequence of SEQ ID NO: 1183;
(3). a first polypeptide comprising the amino acid sequence of SEQ ID NO: 1184, and a second polypeptide comprising the amino acid sequence of SEQ ID NO: 1185;
(4). a first polypeptide comprising the amino acid sequence of SEQ ID NO: 1186, and a second polypeptide comprising the amino acid sequence of SEQ ID NO: 1187;
(5). a first polypeptide comprising the amino acid sequence of SEQ ID NO: 1188, and a second polypeptide comprising the amino acid sequence of SEQ ID NO: 1189;
(6). a first polypeptide comprising the amino acid sequence of SEQ ID NO: 1190, and a second polypeptide comprising the amino acid sequence of SEQ ID NO: 1191;
(7). a first polypeptide comprising the amino acid sequence of SEQ ID NO: 1192, and a second polypeptide comprising the amino acid sequence of SEQ ID NO: 1193;
(8). a first polypeptide comprising the amino acid sequence of SEQ ID NO: 1194, and a second polypeptide comprising the amino acid sequence of SEQ ID NO: 1195;
(9). a first polypeptide comprising the amino acid sequence of SEQ ID NO: 1196, and a second polypeptide comprising the amino acid sequence of SEQ ID NO: 1197;
(10). a first polypeptide comprising the amino acid sequence of SEQ ID NO: 1198, and a second polypeptide comprising the amino acid sequence of SEQ ID NO: 1199;
(11). a first polypeptide comprising the amino acid sequence of SEQ ID NO: 1200, and a second polypeptide comprising the amino acid sequence of SEQ ID NO: 1201; or
(12). a first polypeptide comprising the amino acid sequence of SEQ ID NO: 1202, and a second polypeptide comprising the amino acid sequence of SEQ ID NO: 1203.
Embodiment 14. The antibody or antigen binding fragment of any one of embodiments 1-13, wherein the antibody is an IgG.
Embodiment 15. A nucleic acid molecule encoding the antibody or antigen binding fragment of any one of embodiments 1-14.
Embodiment 16. A vector comprising the nucleic acid molecule of embodiment 15.
Embodiment 17. A host cell transformed with the vector of embodiment 16.
Embodiment 18. A composition comprising a therapeutically effective amount of the antibody or antigen binding fragment of any one of embodiments 1-14, the nucleic acid molecule of embodiment 15, or the vector of embodiment 16, and a pharmaceutically acceptable excipient.
Embodiment 19. A method of preventing suppression of an immune cell or activating a response mediated by an immune cell, comprising contacting the immune cell with the antibody or fragment thereof of any one of embodiments 1-14 or the pharmaceutical composition of embodiment 18.
Embodiment 20. The method of embodiment 19, wherein the immune cell is an NK cell, a macrophage, a T cell or a dendric cell.
Embodiment 21. The method of embodiment 20, wherein the T cell is a CD8+ T cell.
Embodiment 22. The method of any one of embodiments 19-21, wherein the immune cell expresses LILRB1 and/or LILRB2.
Embodiment 23. The method of any one of embodiments 19-22, wherein the response mediated by the immune cell is an anti-tumor response, optionally wherein the tumor cell expresses HLA-A2, HLA-G, and/or ANGPTLs.
Embodiment 24. A method for treating a disease or disorder in a subject comprising administering to the subject the antibody or antigen binding fragment of any one of embodiments 1-14, or the pharmaceutical composition of embodiment 18.
Embodiment 25. The method of embodiment 24, wherein the disease or disorder is a cancer, optionally the cancer expresses HLA-A2, HLA-G and/or ANGPTLs.
Embodiment 26. The method of embodiment 24, wherein the disease or disorder is a solid tumor or a blood tumor.
Embodiment 27. The method of any one of embodiments 24-26, wherein the subject is a human subject.
Embodiment 28. The method of embodiment 24, wherein the antibody or antigen binding fragment is used as part of a combination therapy.
Embodiment 29. The method of embodiment 28, wherein the antibody or antigen binding fragment is used in combination with a PD-1 inhibitor, wherein optionally, the PD-1 inhibitor is an anti-PD-1 antibody, wherein optionally the anti-PD-1 antibody is Nivolumab.
Embodiment 30. The method of embodiment 28, wherein the antibody or antigen binding fragment is used in combination with a PD-L1 inhibitor, wherein optionally, the PD-L1 inhibitor is an anti-PD-L1 antibody, wherein optionally the anti-PD-L1 antibody is LAE005.
Embodiment 31. The method of embodiment 28, wherein the antibody or antigen binding fragment is used in combination with an anti-CD47 antibody, wherein optionally the anti-CD47 antibody is Hu5F9-G4.
Embodiment 32. The method of embodiment 28, wherein the antibody or antigen binding fragment is used in combination with an anti-CD3 antibody, wherein optionally the anti-CD3 antibody is OKT3.

The invention is generally disclosed herein using affirmative language to describe the numerous embodiments. The invention also specifically includes embodiments in which particular subject matter is excluded, in full or in part, such as substances or materials, method steps and conditions, protocols, procedures, assays or analysis. Thus, even though the invention is generally not expressed herein in terms of what the invention does not include, aspects that are not expressly included in the invention are nevertheless disclosed herein.

A number of embodiments of the invention have been described. Nevertheless, it will be understood that various modifications may be made without departing from the spirit and scope of the invention. Accordingly, the following examples are intended to illustrate but not limit the scope of invention described in the claims.

### 7. EXAMPLES

The following is a description of various methods and materials used in the studies, and are put forth so as to provide those of ordinary skill in the art with a complete disclosure and description of how to make and use the present disclosure, and are not intended to limit the scope of what the inventors regard as their disclosure nor are they intended to represent that the experiments below were performed and are all of the experiments that may be performed. It is to be understood that exemplary descriptions written in the present tense were not necessarily performed, but rather that the descriptions can be performed to generate the data and the like associated with the teachings of the present disclosure. Efforts have been made to ensure accuracy with respect to numbers used (e.g., amounts, percentages, etc.), but some experimental errors and deviations should be accounted for.

### 7.1 Example 1: Generation of Anti-LILRB1 and LILRB2 Antibodies

### Immunization and Screening

Immunization was performed by immunizing balb/c and SJL mice. In group 1, the immunogen was human LILRB1-hFc protein (Sino Biological, LLC: Accession: ADJ55949.1, Cat: 16014-H02H). In group 2, the immunogen was human LILRB1-hFc protein and human LILRB1 overexpressing HEK293F cell line (Novobio, LLC: Accession: NM_006669). In group 3, the immunogen was human LILRB1-hFc protein and rhesus macaque LILRB1-hFc protein (Kactusbio, LLC: Accession: NP_001035762.2, LIL-CM1B3). In group 4, the immunogen was human LILRB2-hFc protein (Sino Biological, LLC: Accession: AAH36827.1, Cat: 14132-H02H). In group 5, the immunogen was human LILRB2-hFc protein and human LILRB2 overexpressing HEK293F cell line (Novobio, LLC: Accession: NM_005874). In group 6, the immunogen was human LILRB2-hFc protein and cynomolgus LILRB2-hFc protein (Kactusbio, LLC: Accession: XM_015441717, LIL-CM2B2). Briefly, for each group, 5 Balb/c and 5 SJL mice were immunized with 25-50 μg of protein antigen and/or human LILRB1/LILRB2 overexpressing HEK293F cell line (0.5-1×107 cells/injection). The primary immunization was followed by several boosts until animals developed satisfactory antiserum titers (1:50,000; 3-fold over pre-bleed serum) suitable for hybridoma development.

After the immunization, the mice were sacrificed to perform hybridoma fusion and screening according to the standard protocol. Hybridoma screening was performed to test the binding of the supernatant to human LILRB1-hFc or human LILRB2-hFc protein in ELISA assay and to CHOK1-LILRB1 or CHOK1-LILRB2 cells (Novobio, LLC: Accession: NM_006669/NM_005874) in FACS assay. For ELISA screen, the hybridoma supernatant or the reference antibodies (100 μl/well) was added to the coated plate (1 μg/ml of coating antigen) at 37° C. for 1 hour. After secondary antibody incubation and TMB development, OD450 was measured using Molecular device spectra max plus384. For FACS screen, the hybridoma supernatants or the reference antibodies were diluted with FACS buffer (0.1 ml/well) and added to the 96-well plate containing 2×105 cells/well. The plates were incubated at 4° C. for 1 hour. After staining with fluorescence conjugated secondary antibody (1:1000) at 4° C. for 1 hour, the plates were analyzed by BD FACS Canto™ II.

174 LILRB1-reactive hybridoma clones and 133 LILRB2-reactive hybridoma clones were identified. Briefly, after obtaining the RNA of hybridoma cells, the cloned target sequence was obtained by PCR. Then, the VH and VL were obtained from the bacterial detection electrophoresis and sequenced with M13F/M13R primers. The supernatants of these hybridomas were evaluated for blocking activity in human LILRB1/HLA-G or human LILRB2/HLA-G binding assays. For ELISA assay, the hybridoma supernatant or antibodies (100 μl/well) were mixed with human LILRB1 or human LILRB2 and added to HLA-G (1 ug/ml, Kactusbio, LLC: Accession: P17693-1, Cat: HLG-HM41CT) coated plate. The plates were incubated at 37° C. for 1 hour. After secondary antibody incubation and TMB development, the plates were read at 450 nm using Molecular device spectra max plus384. For FACS screen, the hybridoma supernatants or antibodies were mixed with HLA-G (Creative Biolabs: Accession: P17693-1, Cat: MHC-LC1325) and diluted with FACS buffer. The mixture (0.1 ml/well) was added to the 96-well plate containing CHOK1-LILRB1 or CHOK1-LILRB2 cells (2×10⁵ cells/well). The plates were incubated at 4° C. for 1 hour. After stained with fluorescence dye conjugated secondary antibody (1:1000), the cells were analyzed by BD FACS Canto™ II.

Reference antibodies 12D12 (WO2020136147A1), 15G8 (WO2021028921A9), 3C1 (WO2020023268A1) and hz73D1.v1 (WO2021222544A1) were chosen as the positive control for LILRB1 binding and blocking assays. Reference antibodies p1E1 (WO2018187518A1), J19.h1 (WO2019126514A2), B2-19 (WO2021158413A1) and hz73D1.v1 (WO2021222544A1) were chosen as the positive control for LILRB2 binding and blocking assays. These reference antibodies were produced as human IgG4 (S228P). The sequences of the reference antibodies are listed in Tables 1-3 below. The CDRs were determined by the Kabat system.

Exemplary signal peptides used for antibody expression include without limitation to: MDMRVPAQLLGLLLLWLRGARC (SEQ ID NO: 1207); MGWLWNLLFLMAAAQSAQA (SEQ ID NO: 1208); MVFTPQILGLMLFWISASRG (SEQ ID NO: 1209).

TABLE 1
Heavy chain and Light chain sequences of the reference antibodies

| Antibody | Heavy Chain | Light Chain |
|---|---|---|
| 12D12 | QVQLQQPGAELVKPGASVRMSCKAS | DIVMTQSPASLSVSVGETVTITCRASEN |
| | GYTFTSYWVHWVKQRPGQGLEWIGV | IYSNLAWYQQKQGKSPQLLVYAATNL |
| | IDPSDSYTSYNQNFKGKATLTVDTSSK | ADGVPSRFSGSRSGTQYSLKINSLQSED |
| | TAYIHLSSLTSEDSAVYFCARGERYD | FGTYYCQHFWNTPRTFGGGTKLEIKRT |
| | GDYFAMDYWGQGTSVTVSSASTKGP | VAAPSVFIFPPSDEQLKSGTASVVCLLN |
| | SVFPLAPCSRSTSESTAALGCLVKDYF | NFYPREAKVQWKVDNALQSGNSQESV |
| | PEPVTVSWNSGALTSGVHTFPAVLQS | TEQDSKDSTYSLSSTLTLSKADYEKHK |
| | SGLYSLSSVVTVPSSSLGTKTYTCNVD | VYACEVTHQGLSSPVTKSFNRGEC |
| | HKPSNTKVDKRVESKYGPPCPPCPAP | (SEQ ID NO: 2) |
| | EFLGGPSVFLFPPKPKDTLMISRTPEVT | |
| | CVVVDVSQEDPEVQFNWYVDGVEVH | |
| | NAKTKPREEQFNSTYRVVSVLTVLHQ | |
| | DWLNGKEYKCKVSNKGLPSSIEKTIS | |
| | KAKGQPREPQVYTLPPSQEEMTKNQV | |
| | SLTCLVKGFYPSDIAVEWESNGQPEN | |
| | NYKTTPPVLDSDGSFFLYSRLTVDKSR | |
| | WQEGNVFSCSVMHEALHNHYTQKSL | |
| | SLSLGK (SEQ ID NO: 1) | |
| 15G8 | DVQLQGSGPGLVKPSETLSLTCSVTG | DIQMTQSPSSLSASVGDRVTITCRTSQD |
| | YSITSGYYWNWIRQFPGKKLEWMGYI | ISNYLNWYQQKPGKAVKLLISYTSRLH |
| | SYDGSNNYNPSLKNRITISRDTSKNQF | SGVPSRFSGSGSGTDYTLTISSLQPEDF |
| | SLKLNSVTAADTATYYCAHGYSYYY | ATYYCQQGNTLPTFGQGTKLEIKRTVA |
| | AMDAWGQGTSVTVSSASTKGPSVFPL | APSVFIFPPSDEQLKSGTASVVCLLNNF |
| | APCSRSTSESTAALGCLVKDYFPEPVT | YPREAKVQWKVDNALQSGNSQESVTE |
| | VSWNSGALTSGVHTFPAVLQSSGLYS | QDSKDSTYSLSSTLTLSKADYEKHKVY |
| | LSSVVTVPSSSLGTKTYTCNVDHKPS | ACEVTHQGLSSPVTKSFNRGEC (SEQ |
| | NTKVDKRVESKYGPPCPPCPAPEFLG | ID NO: 4) |
| | GPSVFLFPPKPKDTLMISRTPEVTCVV | |
| | VDVSQEDPEVQFNWYVDGVEVHNA | |
| | KTKPREEQFNSTYRVVSVLTVLHQD | |
| | WLNGKEYKCKVSNKGLPSSIEKTISK | |
| | AKGQPREPQVYTLPPSQEEMTKNQVS | |
| | LTCLVKGFYPSDIAVEWESNGQPENN | |
| | YKTTPPVLDSDGSFFLYSRLTVDKSR | |
| | WQEGNVFSCSVMHEALHNHYTQKSL | |
| | SLSLGK (SEQ ID NO: 3) | |
| 3C1 | EVQLVESGGGLVQPGGSLRLSCAASG | SSELTQDPAVSVALGQTVRITCQGDSL |
| | FTFSDYDMHWVRQATGKGLEWVATI | RSYYESWYQQKPGQAPVLVIYGKNNR |
| | DTAGDTYYPDSVRGRFTISRENAKNS | PSGIPDRFSGSISGNTASLTITGTQAEDE |
| | LSLQMNSLRAGDTAVYFCARDVPGT | ADYYCNSRDNSGDHWVFGGGTRLTV |
| | GFDHWGQGTLVTVSSASTKGPSVFPL | LGQPRTVAAPSVFIFPPSDEQLKSGTAS |
| | APCSRSTSESTAALGCLVKDYFPEPVT | VVCLLNNFYPREAKVQWKVDNALQS |
| | VSWNSGALTSGVHTFPAVLQSSGLYS | GNSQESVTEQDSKDSTYSLSSTLTLSK |
| | LSSVVTVPSSSLGTKTYTCNVDHKPS | ADYEKHKVYACEVTHQGLSSPVTKSF |
| | NTKVDKRVESKYGPPCPSCPAPEFLG | NRGEC (SEQ ID NO: 6) |
| | GPSVFLFPPKPKDTLMISRTPEVTCVV | |
| | VDVSQEDPEVQFNWYVDGVEVHNA | |
| | KTKPREEQFNSTYRVVSVLTVLHQD | |
| | WLNGKEYKCKVSNKGLPSSIEKTISK | |
| | AKGQPREPQVYTLPPSQEEMTKNQVS | |
| | LTCLVKGFYPSDIAVEWESNGQPENN | |
| | YKTTPPVLDSDGSFFLYSRLTVDKSR | |
| | WQEGNVFSCSVMHEALHNHYTQKSL | |
| | SLSLGK (SEQ ID NO: 5) | |
| p1E1 | EVQLQQWGAGLLKPSETLSLTCAVY | ESVLTQPPSVSGAPGQRVTISCTGSSSN |
| | GGSFSGYYWSWIRQPPGKGLEWIGEI | IGAGYDVHWYQQLPGTAPKLLIYGNS |
| | NHSGSTNYNPSLKSRVTISVDTSKNQF | NRPSGVPDRFSVSKSGASASLAITGLQ |
| | SLKLSSVTAADTAVYYCARLPTRWVT | AEDEADYYCQSFDNSLSAYVFGGGTQ |
| | TRYFDLWGRGTLVTVSSASTKGPSVF | LTVLGQPRTVAAPSVFIFPPSDEQLKSG |
| | PLAPCSRSTSESTAALGCLVKDYFPEP | TASVVCLLNNFYPREAKVQWKVDNA |
| | VTVSWNSGALTSGVHTFPAVLQSSGL | LQSGNSQESVTEQDSKDSTYSLSSTLTL |
| | YSLSSVVTVPSSSLGTKTYTCNVDHK | SKADYEKHKVYACEVTHQGLSSPVTK |
| | PSNTKVDKRVESKYGPPCPPCPAPEFL | SFNRGEC (SEQ ID NO: 8) |
| | GGPSVFLFPPKPKDTLMISRTPEVTCV | |
| | VVDVSQEDPEVQFNWYVDGVEVHN | |
| | AKTKPREEQFNSTYRVVSVLTVLHQD | |
| | WLNGKEYKCKVSNKGLPSSIEKTISK | |
| | AKGQPREPQVYTLPPSQEEMTKNQVS | |
| | LTCLVKGFYPSDIAVEWESNGQPENN | |
| | YKTTPPVLDSDGSFFLYSRLTVDKSR | |
| | WQEGNVFSCSVMHEALHNHYTQKSL | |
| | SLSLGK (SEQ ID NO: 7) | |
| J19.h1 | QITLKESGPTLVKPTQTLTLTCTFSGFS | DIQMTQSPSSLSTSVGDRVTITCRASED |
| | LNTYAMGVSWIRQPPGKALEWLASI | IYNDLAWYQQKPGKAPKLLIYNANSL |
| | WWNGNKYNNPSLKSRLTVTKDTSKN | HTGVASRFSGSGSGTDFTFTISSLQPED |
| | QVVLTMTNMDPVDTATYYCAHSRIIR | VATYFCQQYYDYPLTFGQGTKLEIKRT |
| | FTDYVMDAWGQGTLVTVSSASTKGP | VAAPSVFIFPPSDEQLKSGTASVVCLLN |
| | SVFPLAPCSRSTSESTAALGCLVKDYF | NFYPREAKVQWKVDNALQSGNSQESV |
| | PEPVTVSWNSGALTSGVHTFPAVLQS | TEQDSKDSTYSLSSTLTLSKADYEKHK |
| | SGLYSLSSVVTVPSSSLGTKTYTCNVD | VYACEVTHQGLSSPVTKSFNRGEC |
| | HKPSNTKVDKRVESKYGPPCPPCPAP | (SEQ ID NO: 10) |
| | EFLGGPSVFLFPPKPKDTLMISRTPEVT | |
| | CVVVDVSQEDPEVQFNWYVDGVEVH | |
| | NAKTKPREEQFNSTYRVVSVLTVLHQ | |
| | DWLNGKEYKCKVSNKGLPSSIEKTIS | |
| | KAKGQPREPQVYTLPPSQEEMTKNQV | |
| | SLTCLVKGFYPSDIAVEWESNGQPEN | |
| | NYKTTPPVLDSDGSFFLYSRLTVDKSR | |
| | WQEGNVFSCSVMHEALHNHYTQKSL | |
| | SLSLGK (SEQ ID NO: 9) | |
| B2-19 | QVQLVQSGGGLVKPGGSLRLSCAASG | QPVLTQPPSVSEAPRQRVTISCSGSSSNI |
| | FTFSNAWMSWVRQAPGKGLEWVGRI | GNNVVNWYQQLPGKAPKLLIYYDDLL |
| | KSKTDGGTTDYAAPVKGRFTISRDDS | PSGVSDRFSGSKSGTSASLAISGLQSED |
| | KNTLYLQMNSLKTEDTAVYYCTTDR | EADYYCATWDDSLNGPVFGGGTKLTV |
| | YSSSWYSPAFDIWGQGTTVSVSSAST | LGQPKANPTVTLFPPSSEELQANKATL |
| | KGPSVFPLAPCSRSTSESTAALGCLVK | VCLISDFYPGAVTVAWKADGSPVKAG |
| | DYFPEPVTVSWNSGALTSGVHTFPAV | VETTKPSKQSNNKYAASSYLSLTPEQW |
| | LQSSGLYSLSSVVTVPSSSLGTKTYTC | KSHRSYSCQVTHEGSTVEKTVAPTECS |
| | NVDHKPSNTKVDKRVESKYGPPCPPC | (SEQ ID NO: 12) |
| | PAPEFLGGPSVFLFPPKPKDTLMISRTP | |
| | EVTCVVVDVSQEDPEVQFNWYVDGV | |
| | EVHNAKTKPREEQFNSTYRVVSVLTV | |
| | LHQDWLNGKEYKCKVSNKGLPSSIEK | |
| | TISKAKGQPREPQVYTLPPSQEEMTK | |
| | NQVSLTCLVKGFYPSDIAVEWESNGQ | |
| | PENNYKTTPPVLDSDGSFFLYSRLTVD | |
| | KSRWQEGNVFSCSVMHEALHNHYTQ | |
| | KSLSLSLGK (SEQ ID NO: 11) | |
| hz73D1.v1 | QVQLVQSGAEVKKPGASVKVSCKAS | DIQLTQSPSFLSASVGDRVTITCRASES |
| | GYTFTDYYINWVRQAPGQGLEWMG | VDYYGNSFMYWYQQKPGKAPKLLIYF |
| | NVNPNDGGTTYNQKFKGRVTMTTDT | ASNLESGVPSRFSGSGSGTEFTLTISSLQ |
| | STSTAYMELRSLRSDDTAVYYCARRE | PEDFATYYCQQNNEDPWTFGGGTKVE |
| | IYFYGTIYYYAMDYWGQGTLVTVSS | IKRTVAAPSVFIFPPSDEQLKSGTASVV |
| | ASTKGPSVFPLAPCSRSTSESTAALGC | CLLNNFYPREAKVQWKVDNALQSGNS |
| | LVKDYFPEPVTVSWNSGALTSGVHTF | QESVTEQDSKDSTYSLSSTLTLSKADY |
| | PAVLQSSGLYSLSSVVTVPSSSLGTKT | EKHKVYACEVTHQGLSSPVTKSFNRG |
| | YTCNVDHKPSNTKVDKRVESKYGPP | EC (SEQ ID NO: 14) |
| | CPPCPAPEFLGGPSVFLFPPKPKDTLMI | |
| | SRTPEVTCVVVDVSQEDPEVQFNWY | |
| | VDGVEVHNAKTKPREEQFNSTYRVV | |
| | SVLTVLHQDWLNGKEYKCKVSNKGL | |
| | PSSIEKTISKAKGQPREPQVYTLPPSQE | |
| | EMTKNQVSLTCLVKGFYPSDIAVEWE | |
| | SNGQPENNYKTTPPVLDSDGSFFLYSR | |
| | LTVDKSRWQEGNVFSCSVMHEALHN | |
| | HYTQKSLSLSLGK (SEQ ID NO: 13) | |

TABLE 2
VH and VL sequences of the reference antibodies

| Antibody | VH | VL |
|---|---|---|
| 12D12 | QVQLQQPGAELVKPGASVRMSCKAS | DIVMTQSPASLSVSVGETVTITCRASEN |
| | GYTFTSYWVHWVKQRPGQGLEWIGV | IYSNLAWYQQKQGKSPQLLVYAATNL |
| | IDPSDSYTSYNQNFKGKATLTVDTSSK | ADGVPSRFSGSRSGTQYSLKINSLQSED |
| | TAYIHLSSLTSEDSAVYFCARGERYD | FGTYYCQHFWNTPRTFGGGTKLEIK |
| | GDYFAMDYWGQGTSVTVSS (SEQ ID | (SEQ ID NO: 16) |
| | NO: 15) | |
| 15G8 | DVQLQGSGPGLVKPSETLSLTCSVTG | DIQMTQSPSSLSASVGDRVTITCRTSQD |
| | YSITSGYYWNWIRQFPGKKLEWMGYI | ISNYLNWYQQKPGKAVKLLISYTSRLH |
| | SYDGSNNYNPSLKNRITISRDTSKNQF | SGVPSRFSGSGSGTDYTLTISSLQPEDF |
| | SLKLNSVTAADTATYYCAHGYSYYY | ATYYCQQGNTLPTFGQGTKLEIK (SEQ |
| | AMDAWGQGTSVTVSS (SEQ ID NO: | ID NO: 18) |
| | 17) | |
| 3C1 | EVQLVESGGGLVQPGGSLRLSCAASG | SSELTQDPAVSVALGQTVRITCQGDSL |
| | FTFSDYDMHWVRQATGKGLEWVATI | RSYYESWYQQKPGQAPVLVIYGKNNR |
| | DTAGDTYYPDSVRGRFTISRENAKNS | PSGIPDRFSGSISGNTASLTITGTQAEDE |
| | LSLQMNSLRAGDTAVYFCARDVPGT | ADYYCNSRDNSGDHWVFGGGTRLTV |
| | GFDHWGQGTLVTVSS (SEQ ID NO: | LGQP (SEQ ID NO: 20) |
| | 19) | |
| p1E1 | EVQLQQWGAGLLKPSETLSLTCAVY | ESVLTQPPSVSGAPGQRVTISCTGSSSN |
| | GGSFSGYYWSWIRQPPGKGLEWIGEI | IGAGYDVHWYQQLPGTAPKLLIYGNS |
| | NHSGSTNYNPSLKSRVTISVDTSKNQF | NRPSGVPDRFSVSKSGASASLAITGLQ |
| | SLKLSSVTAADTAVYYCARLPTRWVT | AEDEADYYCQSFDNSLSAYVFGGGTQ |
| | TRYFDLWGRGTLVTVSS (SEQ ID NO: | LTVLGQP (SEQ ID NO: 22) |
| | 21) | |
| J19.h1 | QITLKESGPTLVKPTQTLTLTCTFSGFS | DIQMTQSPSSLSTSVGDRVTITCRASED |
| | LNTYAMGVSWIRQPPGKALEWLASI | IYNDLAWYQQKPGKAPKLLIYNANSL |
| | WWNGNKYNNPSLKSRLTVTKDTSKN | HTGVASRFSGSGSGTDFTFTISSLQPED |
| | QVVLTMTNMDPVDTATYYCAHSRIIR | VATYFCQQYYDYPLTFGQGTKLEIK |
| | FTDYVMDAWGQGTLVTVSS (SEQ ID | (SEQ ID NO: 24) |
| | NO: 23) | |
| B2-19 | QVQLVQSGGGLVKPGGSLRLSCAASG | QPVLTQPPSVSEAPRQRVTISCSGSSSNI |
| | FTFSNAWMSWVRQAPGKGLEWVGRI | GNNVVNWYQQLPGKAPKLLIYYDDLL |
| | KSKTDGGTTDYAAPVKGRFTISRDDS | PSGVSDRFSGSKSGTSASLAISGLQSED |
| | KNTLYLQMNSLKTEDTAVYYCTTDR | EADYYCATWDDSLNGPVFGGGTKLTV |
| | YSSSWYSPAFDIWGQGTTVSVSS | L (SEQ ID NO: 26) |
| | (SEQ ID NO: 25) | |
| hz73D1.v1 | QVQLVQSGAEVKKPGASVKVSCKAS | DIQLTQSPSFLSASVGDRVTITCRASES |
| | GYTFTDYYINWVRQAPGQGLEWMG | VDYYGNSFMYWYQQKPGKAPKLLIYF |
| | NVNPNDGGTTYNQKFKGRVTMTTDT | ASNLESGVPSRFSGSGSGTEFTLTISSLQ |
| | STSTAYMELRSLRSDDTAVYYCARRE | PEDFATYYCQQNNEDPWTFGGGTKVE |
| | IYFYGTIYYYAMDYWGQGTLVTVSS | IK (SEQ ID NO: 28) |
| | (SEQ ID NO: 27) | |

TABLE 3
CDR sequences of the reference antibodies

| Antibody | CDR-H1 | CDR-H2 | CDR-H3 | CDR-L1 | CDR-L2 | CDR-L3 |
|---|---|---|---|---|---|---|
| 12D12 | SYWVH | VIDPSDSY | GERYDGD | RASENIYS | AATNLAD | QHFWNTP |
| | (SEQ ID | TSYNQNF | YFAMDY | NLA (SEQ | (SEQ ID | RT (SEQ |
| | NO: 29) | KG (SEQ | (SEQ ID | ID NO: 32) | NO: 33) | ID NO: 34) |
| | | ID NO: 30) | NO: 31) | | | |
| 15G8 | SGYYWN | YISYDGSN | GYSYYYA | RTSQDISN | YTSRLHS | QQGNTLP |
| | (SEQ ID | NYNPSLK | MDA (SEQ | YLN (SEQ | (SEQ ID | T (SEQ ID |
| | NO: 35) | N (SEQ ID | ID NO: 37) | ID NO: 38) | NO: 39) | NO: 40) |
| | | NO: 36) | | | | |
| 3C1 | DYDMH | TIDTAGDT | DVPGTGF | QGDSLRS | GKNNRPS | NSRDNSG |
| | (SEQ ID | YYPDSVR | DH (SEQ | YYES (SEQ | (SEQ ID | DHWV |
| | NO: 41) | G (SEQ ID | ID NO: 43) | ID NO: 44) | NO: 45) | (SEQ ID |
| | | NO: 42) | | | | NO: 46) |
| p1E1 | GYYWS | EINHSGST | LPTRWVT | TGSSSNIG | GNSNRPS | QSFDNSLS |
| | (SEQ ID | NYNPSLKS | TRYFDL | AGYDVH | (SEQ ID | AYV (SEQ |
| | NO: 47) | (SEQ ID | (SEQ ID | (SEQ ID | NO: 51) | ID NO: 52) |
| | | NO: 48) | NO: 49) | NO: 50) | | |
| J19.h1 | TYAMGVS | SIWWNGN | SRIIRFTDY | RASEDIYN | NANSLHT | QQYYDYP |
| | (SEQ ID | KYNNPSL | VMDA | DLA (SEQ | (SEQ ID | LT (SEQ ID |
| | NO: 53) | KS (SEQ | (SEQ ID | ID NO: 56) | NO: 57) | NO: 58) |
| | | ID NO: 54) | NO: 55) | | | |
| B2-19 | NAWMS | RIKSKTDG | DRYSSSW | SGSSSNIG | YDDLLPS | ATWDDSL |
| | (SEQ ID | GTTDYAA | YSPAFDI | NNVVN | (SEQ ID | NGPV |
| | NO: 59) | PVKG | (SEQ ID | (SEQ ID | NO: 63) | (SEQ ID |
| | | (SEQ ID | NO: 61) | NO: 62) | | NO: 64) |
| | | NO: 60) | | | | |
| Antibody | CDR-H1 | CDR-H2 | CDR-H3 | CDR-L1 | CDR-L2 | CDR-L3 |
| hz73D1.v1 | DYYIN | NVNPNDG | REIYFYGT | RASESVD | FASNLES | QQNNEDP |
| | (SEQ ID | GTTYNQK | IYYYAMD | YYGNSFM | (SEQ ID | WT (SEQ |
| | NO: 65) | FKG (SEQ | Y (SEQ ID | Y (SEQ ID | NO: 69) | ID NO: 70) |
| | | ID NO: 66) | NO: 67) | NO: 68) | | |

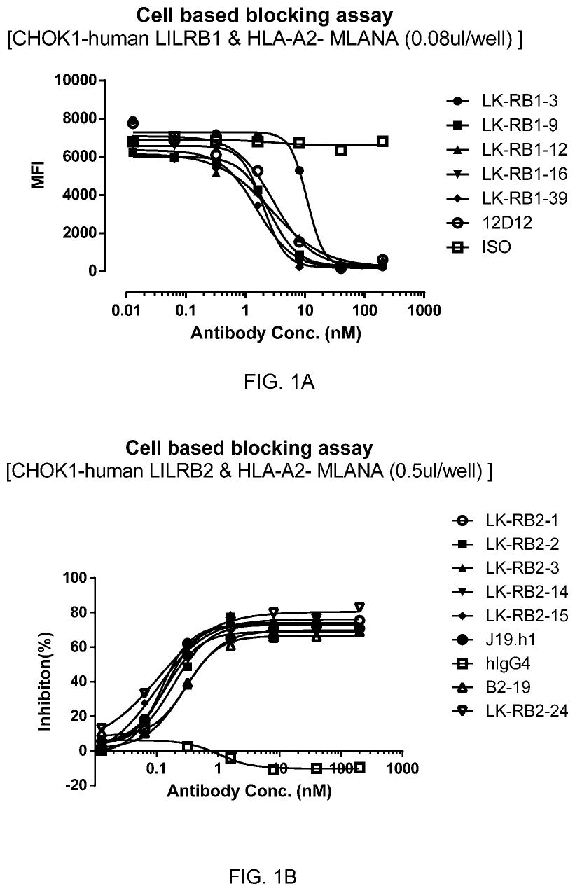

59 anti-LILRB1 antibodies and 47 anti-LILRB2 antibodies with high binding affinity and blocking activity were selected. The sequences of 59 anti-LILRB1 antibodies (LK-RB1-1 to LK-RB1-59) and 47 anti-LILRB2 antibodies (LK-RB2-1 to LK-RB2-47) are shown in Tables 4-6 below. The CDRs were determined by the Kabat system. The binding and blocking potency of the selected antibodies are summarized in Table 7. In addition to blocking the interaction between with HLA-G, anti-LILRB1 antibodies and anti-LILRB2 antibodies could efficiently block the interaction with another ligand HLA-A2 (Creative Biolabs: Accession: P04439-2, Cat: MHC-LC1368) (FIGS. 1A-1B).

Exemplary signal peptides used for antibody expression include without limitation to:

(SEQ ID NO: 1207)
MDMRVPAQLLGLLLLWLRGARC;
(SEQ ID NO: 1208)
MGWLWNLLFLMAAAQSAQA;
(SEQ ID NO: 1209)
MVFTPQILGLMLFWISASRG;
(SEQ ID NO: 1210)
MAWTPLFFFFVLHCSGSFS;
(SEQ ID NO: 1211)
MGVPTQLLLLWLTVVVVRC;
(SEQ ID NO: 1212)
MGWSCIMLFLAATATGVHS;
(SEQ ID NO: 1213)
MESQTQVFVYMLLWLSGVDG;
(SEQ ID NO: 1214)
MGWSYIILFLVATATGVHS;
(SEQ ID NO: 1215)
MDFQVQIFSFLLISASVILSRG;
(SEQ ID NO: 1216)
MESQIQAFVFVFLWLSGVDG;
(SEQ ID NO: 1217)
MNTRAPAEFLGFLLLWFLGARC;
(SEQ ID NO: 1218)
MEWNRVFIFLLSVTAGVHS;
(SEQ ID NO: 1219)
MEFHTQVFVFVFLWLSGVDG;
(SEQ ID NO: 1220)
MDRLTSSFLLLIVPAYVLS;
(SEQ ID NO: 1221)
MVSTPQFLVFLLFWIPASRG;
(SEQ ID NO: 1222)
MGRLTSSFLLLIVPAYVLS;
(SEQ ID NO: 1223)
METDTLLLWVLLLWVPGSTG;
(SEQ ID NO: 1224)
MEWPCIFLFLLSVTEGVHS;
(SEQ ID NO: 1225)
MAVLALLLCLVTFPSCVLS;
(SEQ ID NO: 1226)
MRVLAELLGLLLFCFLGVRC;
(SEQ ID NO: 1227)
MSSAQFLGLLLLCFQGTRC;
(SEQ ID NO: 1228)
MGWSCIILILVVAATGVHS;
(SEQ ID NO: 1229)
MGWSCIILFLVATATGVHS;
(SEQ ID NO: 1230)
MSVPTQVLGLLLLWLTDARC;
(SEQ ID NO: 1231)
MSPAQFLFLLVLWIRENNG;
(SEQ ID NO: 1232)
MGWLWNLLFLMAAAQSAQT;
(SEQ ID NO: 1233)
MERHWIFLSLLSVIAGVHS;
(SEQ ID NO: 1234)
MSVPTQVLGLLLLWLTDVRC;
(SEQ ID NO: 1235)
MELCWVFLFLLPVTAGVHS;
(SEQ ID NO: 1236)
MKVLSLLYLLTAIPGILS;
(SEQ ID NO: 1237)
MGWSCIMLFLAATATGVRS;
(SEQ ID NO: 1238)
MKCSWVIFFLMAVVIGVNS;
(SEQ ID NO: 1239)
MHFQVQIFSFLLISASVIMSRG;
(SEQ ID NO: 1240)
MKLPVRLLVLMFWIPASSS;
(SEQ ID NO: 1241)
MGWSYVILFLVATATGVHS;
(SEQ ID NO: 1242)
MGWSWIFLFLLSGTAGVHS;
(SEQ ID NO: 1243)
MGWSWIFLFLLSGTAGVLS;
(SEQ ID NO: 1244)
METETLLLWVLLLWVPGSTG;
(SEQ ID NO: 1245)
MNLGLSLIFLVLVLKGVQC;
(SEQ ID NO: 1246)
MAVLVLLLCLVTFPSCVLS;
(SEQ ID NO: 1247)
MDFQVQIFSFLLISASVIMSRG;
(SEQ ID NO: 1248)
MEWCWVFLFLLSVTAGVHS;
(SEQ ID NO: 1249)
MDFQVQIFSFLLISISVVMSRG;
(SEQ ID NO: 1250)
MAWLWNLLFLMAAAQSAQA;
(SEQ ID NO: 1251)
MKVLSLLYLLTAIPGFLS;
(SEQ ID NO: 1252)
MDSQAQVLMLLLLWVSGTCG;
(SEQ ID NO: 1253)
MNFGLRLIFLVLTLKGVQC;
(SEQ ID NO: 1254)
MEWSWVSLFFLSVTTGVHS;
(SEQ ID NO: 1255)
MRTPAQFLGILLLWFPGIKC;
(SEQ ID NO: 1256)
MNFGLSLIFLALILKGVQC;
(SEQ ID NO: 1257)
MVSTSQLLGLLLFWTSASRC;
(SEQ ID NO: 1258)
MSVPTQVLGLLLLWLTGARC;
(SEQ ID NO: 1259)
MNFGLSLIFLVLILKGVQC;
(SEQ ID NO: 1260)
MEFRTQVFVFVFLWLSGVDG;
(SEQ ID NO: 1261)
MDMRTPAQFLGILLLWFPGIKC;
(SEQ ID NO: 1262)
METDTLLLWVLLLWVPGSSG;
(SEQ ID NO: 1263)
MGWRYIILFLVATATGVHS;
(SEQ ID NO: 1264)
MGWSCIILILVAAATGVHS;
(SEQ ID NO: 1265)
MESQTLVFISILLWLYGADG;
(SEQ ID NO: 1266)
MKSQTQVFVFLLLCVSGAHG;
(SEQ ID NO: 1267)
MAVLVLFLCLVAFPSCVLS;
(SEQ ID NO: 1268)
MDIRAPAQFLGILLLWFPGARC;
(SEQ ID NO: 1269)
MVLAQFLAFLLLWFPGARC;
(SEQ ID NO: 1270)
MECNWILPFILSVTSGVYS;
(SEQ ID NO: 1271)
MSVLTQVLALLLLWLTGARC;
(SEQ ID NO: 1272)
MNLGLSFIFLALILKGVQC;
(SEQ ID NO: 1273)
MKCSWVIFFLMAVVTGVNS;
(SEQ ID NO: 1274)
MESQIQVFVFVFLWLSGVDG;
(SEQ ID NO: 1275)
MEWSGVFIFLLSVTAGVHS;
(SEQ ID NO: 1276)
MNLGLSLIFLVLVLKGGQC;
(SEQ ID NO: 1277)
MESQTQVLMFLLLWVSGACA;
(SEQ ID NO: 1278)
MGWSWIFLFLLSETAGVLS;
(SEQ ID NO: 1279)
MSPAQFLFLLVLWIRETNG;
(SEQ ID NO: 1280)
MAVLGLLFCLVTLPSCVLS;

TABLE 4
Heavy chain and Light chain sequences of anti-LILRB1 (LK-RB1-1 to LK-
RB1-59) and anti-LILRB2 (LK-RB2-1 to LK-RB2-47) antibodies

| Antibody | Heavy Chain | Light Chain |
|---|---|---|
| LK-RB1-1 | QIQLVQSGPELKKPGETVKISCKASG | QLVLTQSSSASFSLGASAKLTCTLSSQH |
| | YSFTNFGMSWVKQAPGKALKWMG | STYSIDWYQQQPLKPPKYVMELKTDG |
| | WINTYSGVPTYADDFKGRFAFSLETS | SHRTGDGIPDRFSGSSSGADRYLSISNI |
| | ANTAYLQINNLKNEDSATYFCARRR | QPEDEAIYICGVGDTIKEQFVYVFGGG |
| | RTFDGYLCFDYWGQGATLTVSSAST | TKVTVLRTVAAPSVFIFPPSDEQLKSGT |
| | KGPSVFPLAPCSRSTSESTAALGCLV | ASVVCLLNNFYPREAKVQWKVDNAL |
| | KDYFPEPVTVSWNSGALTSGVHTFP | QSGNSQESVTEQDSKDSTYSLSSTLTLS |
| | AVLQSSGLYSLSSVVTVPSSSLGTKT | KADYEKHKVYACEVTHQGLSSPVTKS |
| | YTCNVDHKPSNTKVDKRVESKYGPP | FNRGEC (SEQ ID NO: 72) |
| | CPPCPAPEFLGGPSVFLFPPKPKDTLM | |
| | ISRTPEVTCVVVDVSQEDPEVQFNW | |
| | YVDGVEVHNAKTKPREEQFNSTYRV | |
| | VSVLTVLHQDWLNGKEYKCKVSNK | |
| | GLPSSIEKTISKAKGQPREPQVYTLPP | |
| | SQEEMTKNQVSLTCLVKGFYPSDIAV | |
| | EWESNGQPENNYKTTPPVLDSDGSFF | |
| | LYSRLTVDKSRWQEGNVFSCSVMHE | |
| | ALHNHYTQKSLSLSLGK (SEQ ID | |
| | NO: 71) | |
| LK-RB1-2 | QIQLVQSGPELKKPGETVKISCKASG | DIQMTQSPGSLSASVGETVTITCGASEN |
| | YTFTTYGVSWVKQAPGKGLKWMG | IYGALNWFQRKPGKSPQLLIYGATNLA |
| | WINTYSGVSTYSDDFKGRFAFSLETS | DGMSSRFSGSGSGRQYSLQISSLRPDD |
| | ASTAYLQIINLKNEDAATYFCSRRRR | VATYYCQNVLTTPWTFGGGTKVEIKR |
| | TYDGYLSFDYWGQGTTLTVSSASTK | TVAAPSVFIFPPSDEQLKSGTASVVCLL |
| | GPSVFPLAPCSRSTSESTAALGCLVK | NNFYPREAKVQWKVDNALQSGNSQES |
| | DYFPEPVTVSWNSGALTSGVHTFPA | VTEQDSKDSTYSLSSTLTLSKADYEKH |
| | VLQSSGLYSLSSVVTVPSSSLGTKTY | KVYACEVTHQGLSSPVTKSFNRGEC |
| | TCNVDHKPSNTKVDKRVESKYGPPC | (SEQ ID NO: 74) |
| | PPCPAPEFLGGPSVFLFPPKPKDTLMI | |
| | SRTPEVTCVVVDVSQEDPEVQFNWY | |
| | VDGVEVHNAKTKPREEQFNSTYRVV | |
| | SVLTVLHQDWLNGKEYKCKVSNKG | |
| | LPSSIEKTISKAKGQPREPQVYTLPPS | |
| | QEEMTKNQVSLTCLVKGFYPSDIAV | |
| | EWESNGQPENNYKTTPPVLDSDGSFF | |
| | LYSRLTVDKSRWQEGNVFSCSVMHE | |
| | ALHNHYTQKSLSLSLGK (SEQ ID | |
| | NO: 73) | |
| LK-RB1-3 | QVQLQQPGAELVKPGTSVNLSCKAS | DIVMTQSQKFMPTSVGDRVNVTCKAS |
| | GYTFTSYWIHWVKQRPGRGLEWIGR | QNVGINVAWYQQKLGQSPKALIYSAS |
| | INPYSSGTRYNEKFKNKATLTVDKPS | YRYSGVPDRFTGSGSGTDFTLTINSVQ |
| | STAYMQLTSLTSEDSAVYYCARFDG | SEDLADYFCQQYNNYPFTFGSGTKLEI |
| | APDYFDFWGQGTTLTVSSASTKGPS | KRTVAAPSVFIFPPSDEQLKSGTASVVC |
| | VFPLAPCSRSTSESTAALGCLVKDYF | LLNNFYPREAKVQWKVDNALQSGNS |
| | PEPVTVSWNSGALTSGVHTFPAVLQS | QESVTEQDSKDSTYSLSSTLTLSKADY |
| | SGLYSLSSVVTVPSSSLGTKTYTCNV | EKHKVYACEVTHQGLSSPVTKSFNRG |
| | DHKPSNTKVDKRVESKYGPPCPPCP | EC (SEQ ID NO: 76) |
| | APEFLGGPSVFLFPPKPKDTLMISRTP | |
| | EVTCVVVDVSQEDPEVQFNWYVDG | |
| | VEVHNAKTKPREEQFNSTYRVVSVL | |
| | TVLHQDWLNGKEYKCKVSNKGLPSS | |
| | IEKTISKAKGQPREPQVYTLPPSQEE | |
| | MTKNQVSLTCLVKGFYPSDIAVEWE | |
| | SNGQPENNYKTTPPVLDSDGSFFLYS | |
| | RLTVDKSRWQEGNVFSCSVMHEAL | |
| | HNHYTQKSLSLSLGK (SEQ ID NO: | |
| | 75) | |
| LK-RB1-4 | QVQLQQPGAELVKPGASVKLPCKTS | DIVMTQSQKFMSTSVGDRVSVTCKAS |
| | GYTFTSYWMHWVKQRPGRGLEWIG | QNVGTNVAWYKQKAGQSPKAVIYSA |
| | RIDPHYSGTKYNEKFKNKATMTVDT | SYRYSGVPDRFTGSGSGTDFTLSISNVQ |
| | PSSTAYMQLSSLTSEDSAVYYCARFD | SEDLAEYFCQQYNNNPFTFGSGTKLEI |
| | GDPYYFDYWGPGTTLTVSSASTKGP | KRTVAAPSVFIFPPSDEQLKSGTASVVC |
| | SVFPLAPCSRSTSESTAALGCLVKDY | LLNNFYPREAKVQWKVDNALQSGNS |
| | FPEPVTVSWNSGALTSGVHTFPAVLQ | QESVTEQDSKDSTYSLSSTLTLSKADY |
| | SSGLYSLSSVVTVPSSSLGTKTYTCN | EKHKVYACEVTHQGLSSPVTKSFNRG |
| | VDHKPSNTKVDKRVESKYGPPCPPC | EC (SEQ ID NO: 78) |
| | PAPEFLGGPSVFLFPPKPKDTLMISRT | |
| | PEVTCVVVDVSQEDPEVQFNWYVD | |
| | GVEVHNAKTKPREEQFNSTYRVVSV | |
| | LTVLHQDWLNGKEYKCKVSNKGLP | |
| | SSIEKTISKAKGQPREPQVYTLPPSQE | |
| | EMTKNQVSLTCLVKGFYPSDIAVEW | |
| | ESNGQPENNYKTTPPVLDSDGSFFLY | |
| | SRLTVDKSRWQEGNVFSCSVMHEAL | |
| | HNHYTQKSLSLSLGK (SEQ ID NO: | |
| | 77) | |
| LK-RB1-5 | QVQLQQPGAELVKPGASVKLSCKAS | QIVLSQSPAILSASPGEKVTLTCRASSS |
| | GYTFTDFWIHWVKQRPGQGLEWIG | VSYMHWYQQKPGSSPKPWIYATSNLA |
| | MIHPNSGSTNFNEKFRAKATLTVDKS | SGVPVRFSGSGSGTSYSLTISRMQTED |
| | SSTAYMRLSSLTSEDSAVYYCARSG | AATYYCQQWRRSPYTFGGGTKLEIKR |
| | DGYSNPMDYWGQGTSVTVSSASTK | TVAAPSVFIFPPSDEQLKSGTASVVCLL |
| | GPSVFPLAPCSRSTSESTAALGCLVK | NNFYPREAKVQWKVDNALQSGNSQES |
| | DYFPEPVTVSWNSGALTSGVHTFPA | VTEQDSKDSTYSLSSTLTLSKADYEKH |
| | VLQSSGLYSLSSVVTVPSSSLGTKTY | KVYACEVTHQGLSSPVTKSFNRGEC |
| | TCNVDHKPSNTKVDKRVESKYGPPC | (SEQ ID NO: 80) |
| | PPCPAPEFLGGPSVFLFPPKPKDTLMI | |
| | SRTPEVTCVVVDVSQEDPEVQFNWY | |
| | VDGVEVHNAKTKPREEQFNSTYRVV | |
| | SVLTVLHQDWLNGKEYKCKVSNKG | |
| | LPSSIEKTISKAKGQPREPQVYTLPPS | |
| | QEEMTKNQVSLTCLVKGFYPSDIAV | |
| | EWESNGQPENNYKTTPPVLDSDGSFF | |
| | LYSRLTVDKSRWQEGNVFSCSVMHE | |
| | ALHNHYTQKSLSLSLGK (SEQ ID | |
| | NO: 79) | |
| LK-RB1-6 | QIQLVQSGPELKKPGETVKISCKASG | DIVMTQSHKFMSTSVGDRVSITCTASE |
| | YTFTTYGMSWVKQAPGKDLKWMG | DVNTAVAWYQQKPGQSPKLLIYWAST |
| | WINTFSGVPACADDFQGRFDFSAEPS | RHTGVPDRFTGSGSGTDYTLTISSVQT |
| | DSTAHLQINNLKNEDTATYFCARRS | EDLALYYCQQHYNTPYTFGGGTKLEI |
| | YYGSRGWYFDVWGTGTTVTVSSAS | KRTVAAPSVFIFPPSDEQLKSGTASVVC |
| | TKGPSVFPLAPCSRSTSESTAALGCL | LLNNFYPREAKVQWKVDNALQSGNS |
| | VKDYFPEPVTVSWNSGALTSGVHTF | QESVTEQDSKDSTYSLSSTLTLSKADY |
| | PAVLQSSGLYSLSSVVTVPSSSLGTK | EKHKVYACEVTHQGLSSPVTKSFNRG |
| | TYTCNVDHKPSNTKVDKRVESKYGP | EC (SEQ ID NO: 82) |
| | PCPPCPAPEFLGGPSVFLFPPKPKDTL | |
| | MISRTPEVTCVVVDVSQEDPEVQFN | |
| | WYVDGVEVHNAKTKPREEQFNSTY | |
| | RVVSVLTVLHQDWLNGKEYKCKVS | |
| | NKGLPSSIEKTISKAKGQPREPQVYTL | |
| | PPSQEEMTKNQVSLTCLVKGFYPSDI | |
| | AVEWESNGQPENNYKTTPPVLDSDG | |
| | SFFLYSRLTVDKSRWQEGNVFSCSV | |
| | MHEALHNHYTQKSLSLSLGK (SEQ | |
| | ID NO: 81) | |
| LK-RB1-7 | QIQLVQSGPELKKPGETVKISCKASG | DVQMIQSPSSLSASLGDIVTMTCQASQ |
| | YTFTTYGMSWVKQAPGKDLKWMG | DTTINLNWFQQKPGKVPKLLIYGASNL |
| | WINTFSGVPACADDFQGRFDFSAEPS | EDGVPSRFSGSSFGTDFTLTISSLEDED |
| | DSTAHLQINNLKNEDTATYFCARRS | MATYFCLQHTYLPRTFGGGTKLEIRRT |
| | YYGSRGWYFDVWGTGTTVTVSSAS | VAAPSVFIFPPSDEQLKSGTASVVCLLN |
| | TKGPSVFPLAPCSRSTSESTAALGCL | NFYPREAKVQWKVDNALQSGNSQESV |
| | VKDYFPEPVTVSWNSGALTSGVHTF | TEQDSKDSTYSLSSTLTLSKADYEKHK |
| | PAVLQSSGLYSLSSVVTVPSSSLGTK | VYACEVTHQGLSSPVTKSFNRGEC |
| | TYTCNVDHKPSNTKVDKRVESKYGP | (SEQ ID NO: 84) |
| | PCPPCPAPEFLGGPSVFLFPPKPKDTL | |
| | MISRTPEVTCVVVDVSQEDPEVQFN | |
| | WYVDGVEVHNAKTKPREEQFNSTY | |
| | RVVSVLTVLHQDWLNGKEYKCKVS | |
| | NKGLPSSIEKTISKAKGQPREPQVYTL | |
| | PPSQEEMTKNQVSLTCLVKGFYPSDI | |
| | AVEWESNGQPENNYKTTPPVLDSDG | |
| | SFFLYSRLTVDKSRWQEGNVFSCSV | |
| | MHEALHNHYTQKSLSLSLGK (SEQ | |
| | ID NO: 83) | |
| LK-RB1-8 | QVQLQQSGAELVRPGTSVKVSCKAS | DIVMTQSQKFMSTTVGDRVSITCKASQ |
| | GYAFSNYLIEWVKQRPGQGLEWIGV | NVGTAVVWYQQKPGQSPKLLIYSASN |
| | INPGSDGSTYNEKFKGKATLTADKSS | RYTGVPDRFTGSGSGTDFTLTISNMQS |
| | NTAYMQLSSLTSEDSAVYFCARGDY | EDLADYFCQQYSSYMYTFGGGTKLEI |
| | GRSYGMDYWGQGTSVTVSSASTKG | KRTVAAPSVFIFPPSDEQLKSGTASVVC |
| | PSVFPLAPCSRSTSESTAALGCLVKD | LLNNFYPREAKVQWKVDNALQSGNS |
| | YFPEPVTVSWNSGALTSGVHTFPAVL | QESVTEQDSKDSTYSLSSTLTLSKADY |
| | QSSGLYSLSSVVTVPSSSLGTKTYTC | EKHKVYACEVTHQGLSSPVTKSFNRG |
| | NVDHKPSNTKVDKRVESKYGPPCPP | EC (SEQ ID NO: 86) |
| | CPAPEFLGGPSVFLFPPKPKDTLMISR | |
| | TPEVTCVVVDVSQEDPEVQFNWYVD | |
| | GVEVHNAKTKPREEQFNSTYRVVSV | |
| | LTVLHQDWLNGKEYKCKVSNKGLP | |
| | SSIEKTISKAKGQPREPQVYTLPPSQE | |
| | EMTKNQVSLTCLVKGFYPSDIAVEW | |
| | ESNGQPENNYKTTPPVLDSDGSFFLY | |
| | SRLTVDKSRWQEGNVFSCSVMHEAL | |
| | HNHYTQKSLSLSLGK (SEQ ID NO: | |
| | 85) | |
| LK-RB1-9 | QIQLVQSGPELKKPGETVKISCTASG | DIQMTQSPVSLSASVGETVTITCETSEN |
| | YTFTTYGMSWVKQAPGKGLKWMG | IYGALNWYQRKQGKSPQLLIYGATNL |
| | WINTYSGVPTYADDFKGRFAFSLETS | ADGMSSRFSGSGSGRQYSLEISSLHPD |
| | ASTAYLEINNVKNEDTATYFCARRR | DVASYYCQNVLNTPWTFGGGTKLEIK |
| | RTYHGYLCFDYWGQGTTLTVSSAST | RTVAAPSVFIFPPSDEQLKSGTASVVCL |
| | KGPSVFPLAPCSRSTSESTAALGCLV | LNNFYPREAKVQWKVDNALQSGNSQ |
| | KDYFPEPVTVSWNSGALTSGVHTFP | ESVTEQDSKDSTYSLSSTLTLSKADYE |
| | AVLQSSGLYSLSSVVTVPSSSLGTKT | KHKVYACEVTHQGLSSPVTKSFNRGE |
| | YTCNVDHKPSNTKVDKRVESKYGPP | C (SEQ ID NO: 88) |
| | CPPCPAPEFLGGPSVFLFPPKPKDTLM | |
| | ISRTPEVTCVVVDVSQEDPEVQFNW | |
| | YVDGVEVHNAKTKPREEQFNSTYRV | |
| | VSVLTVLHQDWLNGKEYKCKVSNK | |
| | GLPSSIEKTISKAKGQPREPQVYTLPP | |
| | SQEEMTKNQVSLTCLVKGFYPSDIAV | |
| | EWESNGQPENNYKTTPPVLDSDGSFF | |
| | LYSRLTVDKSRWQEGNVFSCSVMHE | |
| | ALHNHYTQKSLSLSLGK (SEQ ID | |
| | NO: 87) | |
| LK-RB1- | QVTLKESGPGILKPSQTLSLTCSFSGF | DILLTQSPAILSVSPGERVSFSCRASQSI |
| 10 | SLSTSGTGVGWIRQPSGKGLEWLAHI | GTNIHWYQQRMNGSPRLIIKYASELIS |
| | WWNDDKYYKPSLKRQLTISKDTSRN | GIPSRFSGSGSGTDFTLSINSVESEDIAD |
| | QVFLKITSVDTADTATYYCTRTELGR | YYCQQSYGWPYTFGGGTKLETKRTVA |
| | GDYWGQGTSVTVSSASTKGPSVFPL | APSVFIFPPSDEQLKSGTASVVCLLNNF |
| | APCSRSTSESTAALGCLVKDYFPEPV | YPREAKVQWKVDNALQSGNSQESVTE |
| | TVSWNSGALTSGVHTFPAVLQSSGL | QDSKDSTYSLSSTLTLSKADYEKHKVY |
| | YSLSSVVTVPSSSLGTKTYTCNVDHK | ACEVTHQGLSSPVTKSFNRGEC (SEQ |
| | PSNTKVDKRVESKYGPPCPPCPAPEF | ID NO: 90) |
| | LGGPSVFLFPPKPKDTLMISRTPEVTC | |
| | VVVDVSQEDPEVQFNWYVDGVEVH | |
| | NAKTKPREEQFNSTYRVVSVLTVLH | |
| | QDWLNGKEYKCKVSNKGLPSSIEKTI | |
| | SKAKGQPREPQVYTLPPSQEEMTKN | |
| | QVSLTCLVKGFYPSDIAVEWESNGQP | |
| | ENNYKTTPPVLDSDGSFFLYSRLTVD | |
| | KSRWQEGNVFSCSVMHEALHNHYT | |
| | QKSLSLSLGK (SEQ ID NO: 89) | |
| LK-RB1- | QVTLKESGPGILQPSQTLSLTCSFSGF | NIVLTQSPASLAVSLGQRATISCRASES |
| 11 | SLKTSGMSVGWIRQPSGKGLEWLAH | VDSYGNNFIHWYQQKPGQPPKVLIYY |
| | IWWNDDKYYNPVLKGRLTISKDTSN | ASNLEAGVPARFSGSGSRTDFTLTIDPV |
| | NLVFLKIASVVSTDTATYYCVRYGN | EAEDAATYYCQQNNEDPYTFGGGTKL |
| | SHGYWYFDVWGTGTTVTVSSASTK | EIERTVAAPSVFIFPPSDEQLKSGTASV |
| | GPSVFPLAPCSRSTSESTAALGCLVK | VCLLNNFYPREAKVQWKVDNALQSG |
| | DYFPEPVTVSWNSGALTSGVHTFPA | NSQESVTEQDSKDSTYSLSSTLTLSKA |
| | VLQSSGLYSLSSVVTVPSSSLGTKTY | DYEKHKVYACEVTHQGLSSPVTKSFN |
| | TCNVDHKPSNTKVDKRVESKYGPPC | RGEC (SEQ ID NO: 92) |
| | PPCPAPEFLGGPSVFLFPPKPKDTLMI | |
| | SRTPEVTCVVVDVSQEDPEVQFNWY | |
| | VDGVEVHNAKTKPREEQFNSTYRVV | |
| | SVLTVLHQDWLNGKEYKCKVSNKG | |
| | LPSSIEKTISKAKGQPREPQVYTLPPS | |
| | QEEMTKNQVSLTCLVKGFYPSDIAV | |
| | EWESNGQPENNYKTTPPVLDSDGSFF | |
| | LYSRLTVDKSRWQEGNVFSCSVMHE | |
| | ALHNHYTQKSLSLSLGK (SEQ ID | |
| | NO: 91) | |
| LK-RB1- | QVQLQQSGTELVRPGSSVMISCKASG | DIVMTQSHKFMSTSIGDRVSITCKASQ |
| 12 | YAFSTYWMDWVKQRPGQGLEWIGQ | DVSSAVAWYQQKPGQSPKLLIYWAST |
| | IYPGDGATNYNGKFKGKATLTADKS | RHTGVPDRFTGSGSGTDYTLTFSSVQA |
| | SITAYMHLSSLTSEDSAVYFCLRSRD | EDLALYYCQQHYSTPWTFGGGTKLEI |
| | YDGWFFDVWGAGTTVTVSSASTKG | KRTVAAPSVFIFPPSDEQLKSGTASVVC |
| | PSVFPLAPCSRSTSESTAALGCLVKD | LLNNFYPREAKVQWKVDNALQSGNS |
| | YFPEPVTVSWNSGALTSGVHTFPAVL | QESVTEQDSKDSTYSLSSTLTLSKADY |
| | QSSGLYSLSSVVTVPSSSLGTKTYTC | EKHKVYACEVTHQGLSSPVTKSFNRG |
| | NVDHKPSNTKVDKRVESKYGPPCPP | EC (SEQ ID NO: 94) |
| | CPAPEFLGGPSVFLFPPKPKDTLMISR | |
| | TPEVTCVVVDVSQEDPEVQFNWYVD | |
| | GVEVHNAKTKPREEQFNSTYRVVSV | |
| | LTVLHQDWLNGKEYKCKVSNKGLP | |
| | SSIEKTISKAKGQPREPQVYTLPPSQE | |
| | EMTKNQVSLTCLVKGFYPSDIAVEW | |
| | ESNGQPENNYKTTPPVLDSDGSFFLY | |
| | SRLTVDKSRWQEGNVFSCSVMHEAL | |
| | HNHYTQKSLSLSLGK (SEQ ID NO: | |
| | 93) | |
| LK-RB1- | QVTLKESGPGIVKPSQTLSLTCSFSGF | DILLTQSPAILSVSPGDRVSFSCRASQST |
| 13 | SLSISGTGIGWIRQPSGKGLEWLAHI | GTNIHWYQRRTNGSPRLLIKYGSESISG |
| | WWDDDKYYNPSLKSQVTISKDTSTK | IPSRFSGSGTGTDFTLTINSVESEDIADY |
| | QVFLNITSVDTADTATYYCTRTELGR | YCQQSYSWPYTFGGGTKLEIKRTVAA |
| | GDFWGQGTSVTVSSASTKGPSVFPL | PSVFIFPPSDEQLKSGTASVVCLLNNFY |
| | APCSRSTSESTAALGCLVKDYFPEPV | PREAKVQWKVDNALQSGNSQESVTEQ |
| | TVSWNSGALTSGVHTFPAVLQSSGL | DSKDSTYSLSSTLTLSKADYEKHKVYA |
| | YSLSSVVTVPSSSLGTKTYTCNVDHK | CEVTHQGLSSPVTKSFNRGEC (SEQ ID |
| | PSNTKVDKRVESKYGPPCPPCPAPEF | NO: 96) |
| | LGGPSVFLFPPKPKDTLMISRTPEVTC | |
| | VVVDVSQEDPEVQFNWYVDGVEVH | |
| | NAKTKPREEQFNSTYRVVSVLTVLH | |
| | QDWLNGKEYKCKVSNKGLPSSIEKTI | |
| | SKAKGQPREPQVYTLPPSQEEMTKN | |
| | QVSLTCLVKGFYPSDIAVEWESNGQP | |
| | ENNYKTTPPVLDSDGSFFLYSRLTVD | |
| | KSRWQEGNVFSCSVMHEALHNHYT | |
| | QKSLSLSLGK (SEQ ID NO: 95) | |
| LK-RB1- | QVTLKESGPGILKPSQTLSLTCSFSGF | DILLTQSPAILSVSPGERVSFSCRASQSI |
| 14 | SLSTSGMGVGWIRQPSGKGLEWLAH | GTSIHWYQQRTNGSPRLLIKYASESISG |
| | IWWDDDKYYNPSLKSQLTISKDTSR | IPSRFSGSGSGTDFTLSINSVESEDIADY |
| | NQVFLKITSVDTADTATYYCARTEL | YCQQSYSWPYTFGGGTKLEIKRTVAA |
| | GRGDSWGQGTSVTVSSASTKGPSVF | PSVFIFPPSDEQLKSGTASVVCLLNNFY |
| | PLAPCSRSTSESTAALGCLVKDYFPE | PREAKVQWKVDNALQSGNSQESVTEQ |
| | PVTVSWNSGALTSGVHTFPAVLQSS | DSKDSTYSLSSTLTLSKADYEKHKVYA |
| | GLYSLSSVVTVPSSSLGTKTYTCNVD | CEVTHQGLSSPVTKSFNRGEC (SEQ ID |
| | HKPSNTKVDKRVESKYGPPCPPCPAP | NO: 98) |
| | EFLGGPSVFLFPPKPKDTLMISRTPEV | |
| | TCVVVDVSQEDPEVQFNWYVDGVE | |
| | VHNAKTKPREEQFNSTYRVVSVLTV | |
| | LHQDWLNGKEYKCKVSNKGLPSSIE | |
| | KTISKAKGQPREPQVYTLPPSQEEMT | |
| | KNQVSLTCLVKGFYPSDIAVEWESN | |
| | GQPENNYKTTPPVLDSDGSFFLYSRL | |
| | TVDKSRWQEGNVFSCSVMHEALHN | |
| | HYTQKSLSLSLGK (SEQ ID NO: 97) | |
| LK-RB1- | QVTLKESGPGIVKPSQTLSLTCSFSGF | DILLTQSPAILSVSPGDRVSFSCRASQSI |
| 15 | SLSISGTGIGWIRQPSGKGLEWLAHI | GTNIHWYQRRTNGSPRLLIKYGSESISG |
| | WWDDDKYYNPSLKSQVTISKDTSTK | IPSRFSGSGTGTDFTLTINSVESEDIADY |
| | QVFLNITSVDTADTATYYCTRTELGR | YCQQSYSWPYTFGGGTKLEIKRTVAA |
| | GDFWGQGTSVTVSSASTKGPSVFPL | PSVFIFPPSDEQLKSGTASVVCLLNNFY |
| | APCSRSTSESTAALGCLVKDYFPEPV | PREAKVQWKVDNALQSGNSQESVTEQ |
| | TVSWNSGALTSGVHTFPAVLQSSGL | DSKDSTYSLSSTLTLSKADYEKHKVYA |
| | YSLSSVVTVPSSSLGTKTYTCNVDHK | CEVTHQGLSSPVTKSFNRGEC (SEQ ID |
| | PSNTKVDKRVESKYGPPCPPCPAPEF | NO: 100) |
| | LGGPSVFLFPPKPKDTLMISRTPEVTC | |
| | VVVDVSQEDPEVQFNWYVDGVEVH | |
| | NAKTKPREEQFNSTYRVVSVLTVLH | |
| | QDWLNGKEYKCKVSNKGLPSSIEKTI | |
| | SKAKGQPREPQVYTLPPSQEEMTKN | |
| | QVSLTCLVKGFYPSDIAVEWESNGQP | |
| | ENNYKTTPPVLDSDGSFFLYSRLTVD | |
| | KSRWQEGNVFSCSVMHEALHNHYT | |
| | QKSLSLSLGK (SEQ ID NO: 99) | |
| LK-RB1- | QVQLKESGPGLVAPSQSLSITCTVSG | DIQMNQSPSSLSASLGDTITITCHASQS |
| 16 | FSLISNAISWVRQPPGKGLEWLGVIW | VDFWLNWYQQKPGNIPKLLISKASNL |
| | GDGNTIYHSALRSRLTISKDNSKSQV | HTGVPSRFSGSGSGTTFTLTISSLQPEDI |
| | FLKLNSLQIDDTATYYCAKANWDGY | ATYYCQQGQSYPLTFGSGTKLEIKRTV |
| | YFDYWGQGTTLTVSSASTKGPSVFPL | AAPSVFIFPPSDEQLKSGTASVVCLLNN |
| | APCSRSTSESTAALGCLVKDYFPEPV | FYPREAKVQWKVDNALQSGNSQESVT |
| | TVSWNSGALTSGVHTFPAVLQSSGL | EQDSKDSTYSLSSTLTLSKADYEKHKV |
| | YSLSSVVTVPSSSLGTKTYTCNVDHK | YACEVTHQGLSSPVTKSFNRGEC (SEQ |
| | PSNTKVDKRVESKYGPPCPPCPAPEF | ID NO: 102) |
| | LGGPSVFLFPPKPKDTLMISRTPEVTC | |
| | VVVDVSQEDPEVQFNWYVDGVEVH | |
| | NAKTKPREEQFNSTYRVVSVLTVLH | |
| | QDWLNGKEYKCKVSNKGLPSSIEKTI | |
| | SKAKGQPREPQVYTLPPSQEEMTKN | |
| | QVSLTCLVKGFYPSDIAVEWESNGQP | |
| | ENNYKTTPPVLDSDGSFFLYSRLTVD | |
| | KSRWQEGNVFSCSVMHEALHNHYT | |
| | QKSLSLSLGK (SEQ ID NO: 101) | |
| LK-RB1- | QVQLQQPGAELVKPGASVKLSCEAS | DIVMTQSQKFMSTSIGDRVSVTCKASQ |
| 17 | GYTFTNYWIHWVKQRPGRGLEWIGR | NVDTNVAWYQQKPGQSPKALIYSASY |
| | IAPNSGGTKYNEKFKTRATLTVDKAS | RYSGIPDRFTGSGSGTDFTLTISNVQSE |
| | TTAYMQLSSLTSEDSAVYYCARLDG | DLAEYFCQQYNSYPYTFGGGTKLEIKR |
| | SPYYFDYWGQGTTLTVSSASTKGPS | TVAAPSVFIFPPSDEQLKSGTASVVCLL |
| | VFPLAPCSRSTSESTAALGCLVKDYF | NNFYPREAKVQWKVDNALQSGNSQES |
| | PEPVTVSWNSGALTSGVHTFPAVLQS | VTEQDSKDSTYSLSSTLTLSKADYEKH |
| | SGLYSLSSVVTVPSSSLGTKTYTCNV | KVYACEVTHQGLSSPVTKSFNRGEC |
| | DHKPSNTKVDKRVESKYGPPCPPCP | (SEQ ID NO: 104) |
| | APEFLGGPSVFLFPPKPKDTLMISRTP | |
| | EVTCVVVDVSQEDPEVQFNWYVDG | |
| | VEVHNAKTKPREEQFNSTYRVVSVL | |
| | TVLHQDWLNGKEYKCKVSNKGLPSS | |
| | IEKTISKAKGQPREPQVYTLPPSQEE | |
| | MTKNQVSLTCLVKGFYPSDIAVEWE | |
| | SNGQPENNYKTTPPVLDSDGSFFLYS | |
| | RLTVDKSRWQEGNVFSCSVMHEAL | |
| | HNHYTQKSLSLSLGK (SEQ ID NO: | |
| | 103) | |
| LK-RB1- | QVQLKESGPGLVAPSQSLSITCTVSG | DIQMTQTPSSLSASLGDRVTISCRASQD |
| 18 | FSLTNSGVTWIRQPPGKGLEWLGVI | ISNYLNWYQQKPDGTVKLLIYFTSRLH |
| | WGDGSTNYHSALISRLSISKDNSKSQ | SGVPSRFSGSGSGTDYSLTISNLEQEDF |
| | VFLKLNSLQTDDTATYYCAKPHWD | ATYFCQQGITLPLTFGAGTKLELKRTV |
| | YYNMDYWGQGTSVTVSSASTKGPS | AAPSVFIFPPSDEQLKSGTASVVCLLNN |
| | VFPLAPCSRSTSESTAALGCLVKDYF | FYPREAKVQWKVDNALQSGNSQESVT |
| | PEPVTVSWNSGALTSGVHTFPAVLQS | EQDSKDSTYSLSSTLTLSKADYEKHKV |
| | SGLYSLSSVVTVPSSSLGTKTYTCNV | YACEVTHQGLSSPVTKSFNRGEC (SEQ |
| | DHKPSNTKVDKRVESKYGPPCPPCP | ID NO: 106) |
| | APEFLGGPSVFLFPPKPKDTLMISRTP | |
| | EVTCVVVDVSQEDPEVQFNWYVDG | |
| | VEVHNAKTKPREEQFNSTYRVVSVL | |
| | TVLHQDWLNGKEYKCKVSNKGLPSS | |
| | IEKTISKAKGQPREPQVYTLPPSQEE | |
| | MTKNQVSLTCLVKGFYPSDIAVEWE | |
| | SNGQPENNYKTTPPVLDSDGSFFLYS | |
| | RLTVDKSRWQEGNVFSCSVMHEAL | |
| | HNHYTQKSLSLSLGK (SEQ ID NO: | |
| | 105) | |
| LK-RB1- | QVQLKESGPGLVAPSQNLSITCTVSG | DIQMTQTTSSLSASLGDRVTISCRASQD |
| 19 | FSLTSYGVSWVRQPPGKGLEWLGII | ISNFLNWYQQKPDGTVKLLIYSTSRLH |
| | WGDGNTDYHSALISRLSIRKDNSKSQ | SGVPSRFSGSGSGTDYSLTIANLEQEDV |
| | VFLKLNSLKSDDTATYYCAKPHWEY | ATYFCQQGNTLPLTFGAGTKLELKRTV |
| | YTMDYWGQGTSVTVSSASTKGPSVF | AAPSVFIFPPSDEQLKSGTASVVCLLNN |
| | PLAPCSRSTSESTAALGCLVKDYFPE | FYPREAKVQWKVDNALQSGNSQESVT |
| | PVTVSWNSGALTSGVHTFPAVLQSS | EQDSKDSTYSLSSTLTLSKADYEKHKV |
| | GLYSLSSVVTVPSSSLGTKTYTCNVD | YACEVTHQGLSSPVTKSFNRGEC (SEQ |
| | HKPSNTKVDKRVESKYGPPCPPCPAP | ID NO: 108) |
| | EFLGGPSVFLFPPKPKDTLMISRTPEV | |
| | TCVVVDVSQEDPEVQFNWYVDGVE | |
| | VHNAKTKPREEQFNSTYRVVSVLTV | |
| | LHQDWLNGKEYKCKVSNKGLPSSIE | |
| | KTISKAKGQPREPQVYTLPPSQEEMT | |
| | KNQVSLTCLVKGFYPSDIAVEWESN | |
| | GQPENNYKTTPPVLDSDGSFFLYSRL | |
| | TVDKSRWQEGNVFSCSVMHEALHN | |
| | HYTQKSLSLSLGK (SEQ ID NO: 107) | |
| LK-RB1- | QVQLKESGPGLVAPSQSLSITCTVSG | DIQMNQSPSSLSASLGDTITITCHASQNI |
| 20 | FSLNSNGVSWVRQPPGKGLEWLGLI | DFWLSWYQQKPGNIPKLLIYKASNLHT |
| | WGDGSTNYHSALISRLSIRKDNSKSQ | GVPSRFSGSGSGTGFTLTISSLQPEDIAT |
| | VFLKLNSLQNDDTATYYCAKPNWD | YYCQQGQSYPLTFGAGTKLELKRTVA |
| | GYHFDYWGQGTTLIVSSASTKGPSVF | APSVFIFPPSDEQLKSGTASVVCLLNNF |
| | PLAPCSRSTSESTAALGCLVKDYFPE | YPREAKVQWKVDNALQSGNSQESVTE |
| | PVTVSWNSGALTSGVHTFPAVLQSS | QDSKDSTYSLSSTLTLSKADYEKHKVY |
| | GLYSLSSVVTVPSSSLGTKTYTCNVD | ACEVTHQGLSSPVTKSFNRGEC (SEQ |
| | HKPSNTKVDKRVESKYGPPCPPCPAP | ID NO: 110) |
| | EFLGGPSVFLFPPKPKDTLMISRTPEV | |
| | TCVVVDVSQEDPEVQFNWYVDGVE | |
| | VHNAKTKPREEQFNSTYRVVSVLTV | |
| | LHQDWLNGKEYKCKVSNKGLPSSIE | |
| | KTISKAKGQPREPQVYTLPPSQEEMT | |
| | KNQVSLTCLVKGFYPSDIAVEWESN | |
| | GQPENNYKTTPPVLDSDGSFFLYSRL | |
| | TVDKSRWQEGNVFSCSVMHEALHN | |
| | HYTQKSLSLSLGK (SEQ ID NO: 109) | |
| LK-RB1- | QVQLQQPGAELVKPGASVKMSCKAS | DIVMTQSHKFMSTSIGDRVSITCKASQ |
| 21 | GYIFTDYWTTWVKQRPGQGLEWIGD | DVSTAVAWCQQKPGQSPKSLIYSASY |
| | IYPVSGSTNYNEKFKNKATLTVDTSS | RYTGIPDRFTGSGSGTDFTFTISSVQAE |
| | STAYMQLSSLTSEDSAVYYCARALG | DLAVYYCQQHYTNPVTFGTGTKLELK |
| | RRYYFDYWGQGTTLTVSSASTKGPS | RTVAAPSVFIFPPSDEQLKSGTASVVCL |
| | VFPLAPCSRSTSESTAALGCLVKDYF | LNNFYPREAKVQWKVDNALQSGNSQ |
| | PEPVTVSWNSGALTSGVHTFPAVLQS | ESVTEQDSKDSTYSLSSTLTLSKADYE |
| | SGLYSLSSVVTVPSSSLGTKTYTCNV | KHKVYACEVTHQGLSSPVTKSFNRGE |
| | DHKPSNTKVDKRVESKYGPPCPPCP | C (SEQ ID NO: 112) |
| | APEFLGGPSVFLFPPKPKDTLMISRTP | |
| | EVTCVVVDVSQEDPEVQFNWYVDG | |
| | VEVHNAKTKPREEQFNSTYRVVSVL | |
| | TVLHQDWLNGKEYKCKVSNKGLPSS | |
| | IEKTISKAKGQPREPQVYTLPPSQEE | |
| | MTKNQVSLTCLVKGFYPSDIAVEWE | |
| | SNGQPENNYKTTPPVLDSDGSFFLYS | |
| | RLTVDKSRWQEGNVFSCSVMHEAL | |
| | HNHYTQKSLSLSLGK (SEQ ID NO: | |
| | 111) | |
| LK-RB1- | QVQLKESGPGLVAPSQSLSITCTVSG | DIQMTQTTSSLSASLGDRVTISCRASQD |
| 22 | FSLTSYGVSWVRQPPGKGLEWLGII | ISNFLNWYQQKPDGTVKLLIYYTSRLH |
| | WGDGNTNYHSALTSRLSISKDNSKS | SGAPSRFSGSGSGTDYSLTISNLDQEDI |
| | QVFLKLNSLRTDDTATYYCAKPHWD | ATYFCQQGNTLPLTFGAGTKLELKRTV |
| | HYSMDYWGQGTSVTVSSASTKGPSV | AAPSVFIFPPSDEQLKSGTASVVCLLNN |
| | FPLAPCSRSTSESTAALGCLVKDYFP | FYPREAKVQWKVDNALQSGNSQESVT |
| | EPVTVSWNSGALTSGVHTFPAVLQSS | EQDSKDSTYSLSSTLTLSKADYEKHKV |
| | GLYSLSSVVTVPSSSLGTKTYTCNVD | YACEVTHQGLSSPVTKSFNRGEC (SEQ |
| | HKPSNTKVDKRVESKYGPPCPPCPAP | ID NO: 114) |
| | EFLGGPSVFLFPPKPKDTLMISRTPEV | |
| | TCVVVDVSQEDPEVQFNWYVDGVE | |
| | VHNAKTKPREEQFNSTYRVVSVLTV | |
| | LHQDWLNGKEYKCKVSNKGLPSSIE | |
| | KTISKAKGQPREPQVYTLPPSQEEMT | |
| | KNQVSLTCLVKGFYPSDIAVEWESN | |
| | GQPENNYKTTPPVLDSDGSFFLYSRL | |
| | TVDKSRWQEGNVFSCSVMHEALHN | |
| | HYTQKSLSLSLGK (SEQ ID NO: 113) | |
| LK-RB1- | QVQLQQTGAELVRPGSSVKLSCKAS | DIQMTQSPASQSVSVGETVTITCRASE |
| 23 | GYTFTNYWMDWVKQRPGQGLEWIG | NIYSNLAWYQQKQGKSPQLLVYGATN |
| | NIHPFGNETHYNQKFKDKATLTVDK | LADGVSLRFSGSGSGTQYSLKINSLQSE |
| | SSSTAYMQLSSLTSEDSAVYYCARGL | DFGSYYCQHFWGIPWTFGGGTKLEIKR |
| | LLYFDVWGTGTTVTVSSASTKGPSV | TVAAPSVFIFPPSDEQLKSGTASVVCLL |
| | FPLAPCSRSTSESTAALGCLVKDYFP | NNFYPREAKVQWKVDNALQSGNSQES |
| | EPVTVSWNSGALTSGVHTFPAVLQSS | VTEQDSKDSTYSLSSTLTLSKADYEKH |
| | GLYSLSSVVTVPSSSLGTKTYTCNVD | KVYACEVTHQGLSSPVTKSFNRGEC |
| | HKPSNTKVDKRVESKYGPPCPPCPAP | (SEQ ID NO: 116) |
| | EFLGGPSVFLFPPKPKDTLMISRTPEV | |
| | TCVVVDVSQEDPEVQFNWYVDGVE | |
| | VHNAKTKPREEQFNSTYRVVSVLTV | |
| | LHQDWLNGKEYKCKVSNKGLPSSIE | |
| | KTISKAKGQPREPQVYTLPPSQEEMT | |
| | KNQVSLTCLVKGFYPSDIAVEWESN | |
| | GQPENNYKTTPPVLDSDGSFFLYSRL | |
| | TVDKSRWQEGNVFSCSVMHEALHN | |
| | HYTQKSLSLSLGK (SEQ ID NO: 115) | |
| LK-RB1- | QVQLQQPGAELVNPGTSVNLSCKAS | DVMMTQTPLTLSVTIGQPASISCKSSQS |
| 24 | GYTFTGYWMHWVKQRPGQGLEWIG | LLNSDGKTYLNWLLQRPGQSPKRLIYL |
| | MIHPNNGGTIYNERFNGKATLTVDRS | VSKLGSGVPDRFTGSGSGTDFTLKISR |
| | STTAYIQLSSLTSEDSAVYYCAYWDL | VEAEDLGVYYCWQGTHFPWTFGGGA |
| | DYWGQGTTLTVSSASTKGPSVFPLAP | KLEIKRTVAAPSVFIFPPSDEQLKSGTA |
| | CSRSTSESTAALGCLVKDYFPEPVTV | SVVCLLNNFYPREAKVQWKVDNALQS |
| | SWNSGALTSGVHTFPAVLQSSGLYSL | GNSQESVTEQDSKDSTYSLSSTLTLSK |
| | SSVVTVPSSSLGTKTYTCNVDHKPSN | ADYEKHKVYACEVTHQGLSSPVTKSF |
| | TKVDKRVESKYGPPCPPCPAPEFLGG | NRGEC (SEQ ID NO: 118) |
| | PSVFLFPPKPKDTLMISRTPEVTCVVV | |
| | DVSQEDPEVQFNWYVDGVEVHNAK | |
| | TKPREEQFNSTYRVVSVLTVLHQDW | |
| | LNGKEYKCKVSNKGLPSSIEKTISKA | |
| | KGQPREPQVYTLPPSQEEMTKNQVS | |
| | LTCLVKGFYPSDIAVEWESNGQPEN | |
| | NYKTTPPVLDSDGSFFLYSRLTVDKS | |
| | RWQEGNVFSCSVMHEALHNHYTQK | |
| | SLSLSLGK (SEQ ID NO: 117) | |
| LK-RB1- | QIQLVQSGPELKKPGETVKISCKASG | DIVMTQSHKFMSTSVGDRVSITCKASQ |
| 25 | YTFTSYGMNWMKQAPGEGLKWMG | DVNTAVAWYQQKPGQSPKLLIYWAST |
| | WINTYSGVPTYADDFKGRFAFSLETS | RHTGVPDRFTGSGSGTDFTLTISSVQAE |
| | ATTVYLQINNLKNEDKATYFCARRG | DLALYYCQQHYTIPFTFGSGTKLEIKRT |
| | RDSVVGHFDVWGTGTTVTVSSASTK | VAAPSVFIFPPSDEQLKSGTASVVCLLN |
| | GPSVFPLAPCSRSTSESTAALGCLVK | NFYPREAKVQWKVDNALQSGNSQESV |
| | DYFPEPVTVSWNSGALTSGVHTFPA | TEQDSKDSTYSLSSTLTLSKADYEKHK |
| | VLQSSGLYSLSSVVTVPSSSLGTKTY | VYACEVTHQGLSSPVTKSFNRGEC |
| | TCNVDHKPSNTKVDKRVESKYGPPC | (SEQ ID NO: 120) |
| | PPCPAPEFLGGPSVFLFPPKPKDTLMI | |
| | SRTPEVTCVVVDVSQEDPEVQFNWY | |
| | VDGVEVHNAKTKPREEQFNSTYRVV | |
| | SVLTVLHQDWLNGKEYKCKVSNKG | |
| | LPSSIEKTISKAKGQPREPQVYTLPPS | |
| | QEEMTKNQVSLTCLVKGFYPSDIAV | |
| | EWESNGQPENNYKTTPPVLDSDGSFF | |
| | LYSRLTVDKSRWQEGNVFSCSVMHE | |
| | ALHNHYTQKSLSLSLGK (SEQ ID | |
| | NO: 119) | |
| LK-RB1- | QVQLKESGPGLVAPSQSLSITCTVSG | DIQMTQTTSSLSASLGDRVTISCRASQD |
| 26 | FSLTSYGVSWVRQPPGKGLEWLGII | ISNFLNWYQQKPDGTFKLLISYTSTLHS |
| | WGDGSTNYHSALISRLSISKDNSKSQ | GVPSRFSGSGSGTDYSLTISNLEQEDVA |
| | VFLKLNSLQTDDTATYYCAKPNWD | TYFCQQGNTLPLTFGAGTKLALKRTV |
| | YYAMDYWGQGTSVTVSSASTKGPS | AAPSVFIFPPSDEQLKSGTASVVCLLNN |
| | VFPLAPCSRSTSESTAALGCLVKDYF | FYPREAKVQWKVDNALQSGNSQESVT |
| | PEPVTVSWNSGALTSGVHTFPAVLQS | EQDSKDSTYSLSSTLTLSKADYEKHKV |
| | SGLYSLSSVVTVPSSSLGTKTYTCNV | YACEVTHQGLSSPVTKSFNRGEC (SEQ |
| | DHKPSNTKVDKRVESKYGPPCPPCP | ID NO: 122) |
| | APEFLGGPSVFLFPPKPKDTLMISRTP | |
| | EVTCVVVDVSQEDPEVQFNWYVDG | |
| | VEVHNAKTKPREEQFNSTYRVVSVL | |
| | TVLHQDWLNGKEYKCKVSNKGLPSS | |
| | IEKTISKAKGQPREPQVYTLPPSQEE | |
| | MTKNQVSLTCLVKGFYPSDIAVEWE | |
| | SNGQPENNYKTTPPVLDSDGSFFLYS | |
| | RLTVDKSRWQEGNVFSCSVMHEAL | |
| | HNHYTQKSLSLSLGK (SEQ ID NO: | |
| | 121) | |
| LK-RB1- | QVQLQQSGPELVKPGASVRMSCKAS | DIQMTQSPASLSVSVGETVTITCRASEN |
| 27 | GYTFTSHYIHWMKQRPGQGLEWIG | IYSNLAWYQQKQGKSPHLLVYAATNL |
| | WIYPGDGHTKYHEKFKDRATLTADT | ADGVPSRFSGSGSGTQYSLKINSLQSE |
| | SSITAYMRLSSLTSEDSAIYYCVYYS | DFGNYYCQHFWGSPWTFGGGTKLEIK |
| | DSWFPYWGQGTLVTVSAASTKGPSV | RTVAAPSVFIFPPSDEQLKSGTASVVCL |
| | FPLAPCSRSTSESTAALGCLVKDYFP | LNNFYPREAKVQWKVDNALQSGNSQ |
| | EPVTVSWNSGALTSGVHTFPAVLQSS | ESVTEQDSKDSTYSLSSTLTLSKADYE |
| | GLYSLSSVVTVPSSSLGTKTYTCNVD | KHKVYACEVTHQGLSSPVTKSFNRGE |
| | HKPSNTKVDKRVESKYGPPCPPCPAP | C (SEQ ID NO: 124) |
| | EFLGGPSVFLFPPKPKDTLMISRTPEV | |
| | TCVVVDVSQEDPEVQFNWYVDGVE | |
| | VHNAKTKPREEQFNSTYRVVSVLTV | |
| | LHQDWLNGKEYKCKVSNKGLPSSIE | |
| | KTISKAKGQPREPQVYTLPPSQEEMT | |
| | KNQVSLTCLVKGFYPSDIAVEWESN | |
| | GQPENNYKTTPPVLDSDGSFFLYSRL | |
| | TVDKSRWQEGNVFSCSVMHEALHN | |
| | HYTQKSLSLSLGK (SEQ ID NO: 123) | |
| LK-RB1- | QVQLQQSGPELVKPGTSVKMSCKAS | DIQMTQSPASLSVSVGETVTITCRASEN |
| 28 | GYTFTSHYIHWMKQRPGQGLDWIG | IYSNLAWYQQKQGKSPQLLVYAATNL |
| | WIYPGDGHTKYHEKFKGKATLTADT | ADGVPSRFSGSGSGTQYSLKINSLQSE |
| | SSITAYMQLSLLTSEDSAVYYCVYYS | DFGNYFCQHFWGSPWTFGGGTKLEIK |
| | DSWFPYWGQGTLVTVSAASTKGPSV | RTVAAPSVFIFPPSDEQLKSGTASVVCL |
| | FPLAPCSRSTSESTAALGCLVKDYFP | LNNFYPREAKVQWKVDNALQSGNSQ |
| | EPVTVSWNSGALTSGVHTFPAVLQSS | ESVTEQDSKDSTYSLSSTLTLSKADYE |
| | GLYSLSSVVTVPSSSLGTKTYTCNVD | KHKVYACEVTHQGLSSPVTKSFNRGE |
| | HKPSNTKVDKRVESKYGPPCPPCPAP | C (SEQ ID NO: 126) |
| | EFLGGPSVFLFPPKPKDTLMISRTPEV | |
| | TCVVVDVSQEDPEVQFNWYVDGVE | |
| | VHNAKTKPREEQFNSTYRVVSVLTV | |
| | LHQDWLNGKEYKCKVSNKGLPSSIE | |
| | KTISKAKGQPREPQVYTLPPSQEEMT | |
| | KNQVSLTCLVKGFYPSDIAVEWESN | |
| | GQPENNYKTTPPVLDSDGSFFLYSRL | |
| | TVDKSRWQEGNVFSCSVMHEALHN | |
| | HYTQKSLSLSLGK (SEQ ID NO: 125) | |
| LK-RB1- | QVQLKESGPGLVAPSQNLAITCTVSG | DIQMTQTASSLFASLGDRVTISCRASQ |
| 29 | FSLTNSGVSWVRQSPGKGLEWLGII | DISNYLNWYQQKPDGSIKLLISYTSRL |
| | WGDGNTNYHSALISRLSISKDNSKSQ | HSGVPSRFSGSGSGTDYSLSISNLEQED |
| | VFLKLNSLQTDDTGTYYCAKPNWD | IATYFCQQGISLPCTFGGGTKLEVNRT |
| | YYALDYWGQGTSVTVSSASTKGPSV | VAAPSVFIFPPSDEQLKSGTASVVCLLN |
| | FPLAPCSRSTSESTAALGCLVKDYFP | NFYPREAKVQWKVDNALQSGNSQESV |
| | EPVTVSWNSGALTSGVHTFPAVLQSS | TEQDSKDSTYSLSSTLTLSKADYEKHK |
| | GLYSLSSVVTVPSSSLGTKTYTCNVD | VYACEVTHQGLSSPVTKSFNRGEC |
| | HKPSNTKVDKRVESKYGPPCPPCPAP | (SEQ ID NO: 128) |
| | EFLGGPSVFLFPPKPKDTLMISRTPEV | |
| | TCVVVDVSQEDPEVQFNWYVDGVE | |
| | VHNAKTKPREEQFNSTYRVVSVLTV | |
| | LHQDWLNGKEYKCKVSNKGLPSSIE | |
| | KTISKAKGQPREPQVYTLPPSQEEMT | |
| | KNQVSLTCLVKGFYPSDIAVEWESN | |
| | GQPENNYKTTPPVLDSDGSFFLYSRL | |
| | TVDKSRWQEGNVFSCSVMHEALHN | |
| | HYTQKSLSLSLGK (SEQ ID NO: 127) | |
| LK-RB1- | QVQLKESGPGLVAPSQSLSITCTVSG | VIQMTQTTSSLSASLGDRVTISCRASQD |
| 30 | FSLTSYGVSWIRQPPGKGLEWLGVI | ISNYVNWYQQRPDGTVKLLISYTSRLH |
| | WGDGNTNYHSALISRLSISKDNSKSQ | SGVPSRFSGSGSGTDYSLTITNLEQEDI |
| | VFLKLNSLQTDDTATYYCAKPSWDY | ATYFCQQGITLPCTFGGGTKLEINRTV |
| | YAMDYWGQGTSVTVSSASTKGPSVF | AAPSVFIFPPSDEQLKSGTASVVCLLNN |
| | PLAPCSRSTSESTAALGCLVKDYFPE | FYPREAKVQWKVDNALQSGNSQESVT |
| | PVTVSWNSGALTSGVHTFPAVLQSS | EQDSKDSTYSLSSTLTLSKADYEKHKV |
| | GLYSLSSVVTVPSSSLGTKTYTCNVD | YACEVTHQGLSSPVTKSFNRGEC (SEQ |
| | HKPSNTKVDKRVESKYGPPCPPCPAP | ID NO: 130) |
| | EFLGGPSVFLFPPKPKDTLMISRTPEV | |
| | TCVVVDVSQEDPEVQFNWYVDGVE | |
| | VHNAKTKPREEQFNSTYRVVSVLTV | |
| | LHQDWLNGKEYKCKVSNKGLPSSIE | |
| | KTISKAKGQPREPQVYTLPPSQEEMT | |
| | KNQVSLTCLVKGFYPSDIAVEWESN | |
| | GQPENNYKTTPPVLDSDGSFFLYSRL | |
| | TVDKSRWQEGNVFSCSVMHEALHN | |
| | HYTQKSLSLSLGK (SEQ ID NO: 129) | |
| LK-RB1- | QVQLKESGPGLVAPSQSLSITCTVSG | DIQMTQTTSSLSASLGDRVTISCRASQD |
| 31 | FSLTSYGVSWVRQPPGKGLEWLGII | ISNYLNWYLQKPDGTVKLLIYYTSRLH |
| | WGDGSTNYHSALKSRLSISKDNSKS | SGVPSRFSGSGSGTDYSLTISNLEQEDI |
| | QVFLKLNSLQTEDTATYYCAKPNWD | ATYFCQQGITLPCTFGGGTKLEIKRTV |
| | YYAMDYWGQGTSVTVSSASTKGPS | AAPSVFIFPPSDEQLKSGTASVVCLLNN |
| | VFPLAPCSRSTSESTAALGCLVKDYF | FYPREAKVQWKVDNALQSGNSQESVT |
| | PEPVTVSWNSGALTSGVHTFPAVLQS | EQDSKDSTYSLSSTLTLSKADYEKHKV |
| | SGLYSLSSVVTVPSSSLGTKTYTCNV | YACEVTHQGLSSPVTKSFNRGEC (SEQ |
| | DHKPSNTKVDKRVESKYGPPCPPCP | ID NO: 132) |
| | APEFLGGPSVFLFPPKPKDTLMISRTP | |
| | EVTCVVVDVSQEDPEVQFNWYVDG | |
| | VEVHNAKTKPREEQFNSTYRVVSVL | |
| | TVLHQDWLNGKEYKCKVSNKGLPSS | |
| | IEKTISKAKGQPREPQVYTLPPSQEE | |
| | MTKNQVSLTCLVKGFYPSDIAVEWE | |
| | SNGQPENNYKTTPPVLDSDGSFFLYS | |
| | RLTVDKSRWQEGNVFSCSVMHEAL | |
| | HNHYTQKSLSLSLGK (SEQ ID NO: | |
| | 131) | |
| LK-RB1- | QVQLQQSGAELAKPGASVKLSCTAS | DIQMTQSPASLSVSVGETVTITCRASEN |
| 32 | GYTFTTYLMHWVKQRPAQGLEWIG | IYSSLAWYQQKQGKSPQLLVYAATNL |
| | YINPSNDFTKYNQKFKDKATLTADTS | ADGVPSRFSGSGSGTQYSLKINSLQSE |
| | SSTAYMQLNSLTFEDSAVYYCARNS | DFGNYYCQHFWGTWAFGGGTKLEIKR |
| | DYDPSYWGQGTTLTVSSASTKGPSV | TVAAPSVFIFPPSDEQLKSGTASVVCLL |
| | FPLAPCSRSTSESTAALGCLVKDYFP | NNFYPREAKVQWKVDNALQSGNSQES |
| | EPVTVSWNSGALTSGVHTFPAVLQSS | VTEQDSKDSTYSLSSTLTLSKADYEKH |
| | GLYSLSSVVTVPSSSLGTKTYTCNVD | KVYACEVTHQGLSSPVTKSFNRGEC |
| | HKPSNTKVDKRVESKYGPPCPPCPAP | (SEQ ID NO: 134) |
| | EFLGGPSVFLFPPKPKDTLMISRTPEV | |
| | TCVVVDVSQEDPEVQFNWYVDGVE | |
| | VHNAKTKPREEQFNSTYRVVSVLTV | |
| | LHQDWLNGKEYKCKVSNKGLPSSIE | |
| | KTISKAKGQPREPQVYTLPPSQEEMT | |
| | KNQVSLTCLVKGFYPSDIAVEWESN | |
| | GQPENNYKTTPPVLDSDGSFFLYSRL | |
| | TVDKSRWQEGNVFSCSVMHEALHN | |
| | HYTQKSLSLSLGK (SEQ ID NO: 133) | |
| LK-RB1- | QIQLVQSGPELKKPGDTVKISCKASG | DIVMTQSHKFMSTSVGDRVSITCKASQ |
| 33 | YTFTSYGMNWVKQAPGEGLKWMG | DVNTAVAWYQQKPGQSPKLLIYWAST |
| | WINTYSGVPTYGDDFKGRFAFSLETS | RHTGVPDRFTGSGSGTDFTLTISSVQAE |
| | ASTAYLQINNLENEDTATYFCARRG | DLALYYCQQHYTIPFTFGSGTRLEIKRT |
| | RDSVVGHFDVWGTGTTVTVSSASTK | VAAPSVFIFPPSDEQLKSGTASVVCLLN |
| | GPSVFPLAPCSRSTSESTAALGCLVK | NFYPREAKVQWKVDNALQSGNSQESV |
| | DYFPEPVTVSWNSGALTSGVHTFPA | TEQDSKDSTYSLSSTLTLSKADYEKHK |
| | VLQSSGLYSLSSVVTVPSSSLGTKTY | VYACEVTHQGLSSPVTKSFNRGEC |
| | TCNVDHKPSNTKVDKRVESKYGPPC | (SEQ ID NO: 136) |
| | PPCPAPEFLGGPSVFLFPPKPKDTLMI | |
| | SRTPEVTCVVVDVSQEDPEVQFNWY | |
| | VDGVEVHNAKTKPREEQFNSTYRVV | |
| | SVLTVLHQDWLNGKEYKCKVSNKG | |
| | LPSSIEKTISKAKGQPREPQVYTLPPS | |
| | QEEMTKNQVSLTCLVKGFYPSDIAV | |
| | EWESNGQPENNYKTTPPVLDSDGSFF | |
| | LYSRLTVDKSRWQEGNVFSCSVMHE | |
| | ALHNHYTQKSLSLSLGK (SEQ ID | |
| | NO: 135) | |
| LK-RB1- | QVQLQQSGAELVKPGASVKLSCKAS | DILLTQSPAILSVSPGERVSFSCRASRYI |
| 34 | GYTFTEYTIHWVKQRSGQGLEWIGW | GTSIHWYQQRTNGSPRLLIKYASESISG |
| | FYPGSDSIKYNEKFKDKATLTADKSS | IPSRFSGSGSGTDFTLSINSVESEDIADY |
| | STVYMELSRLTSEDSAVYFCSRHEGP | YCQQSNTWPLTFGAGTKLELKRTVAA |
| | YFTSQYYFDYWGQGTTLTVSSASTK | PSVFIFPPSDEQLKSGTASVVCLLNNFY |
| | GPSVFPLAPCSRSTSESTAALGCLVK | PREAKVQWKVDNALQSGNSQESVTEQ |
| | DYFPEPVTVSWNSGALTSGVHTFPA | DSKDSTYSLSSTLTLSKADYEKHKVYA |
| | VLQSSGLYSLSSVVTVPSSSLGTKTY | CEVTHQGLSSPVTKSFNRGEC (SEQ ID |
| | TCNVDHKPSNTKVDKRVESKYGPPC | NO: 138) |
| | PPCPAPEFLGGPSVFLFPPKPKDTLMI | |
| | SRTPEVTCVVVDVSQEDPEVQFNWY | |
| | VDGVEVHNAKTKPREEQFNSTYRVV | |
| | SVLTVLHQDWLNGKEYKCKVSNKG | |
| | LPSSIEKTISKAKGQPREPQVYTLPPS | |
| | QEEMTKNQVSLTCLVKGFYPSDIAV | |
| | EWESNGQPENNYKTTPPVLDSDGSFF | |
| | LYSRLTVDKSRWQEGNVFSCSVMHE | |
| | ALHNHYTQKSLSLSLGK (SEQ ID | |
| | NO: 137) | |
| LK-RB1- | DVQLQESGPGLEKPSQSLSLTCSVTG | DIVLTQSPATLSVTPGDSVSLSCRASQS |
| 35 | YSITRGYQWNWIRQFPGNKLEWMG | ISSNLHWYQQKSHESPRLLIKYASQSIS |
| | YISYDGSNNYNPSLKNRISITRDASKN | GIPSRFSGSGSGTDFTLSINSVETEDFG |
| | QFFLKLNSVTTEDTATYYCARRSYNS | MYFCQQSNSWPLTFGAGTKLELKRTV |
| | IDYWGQGTTLTVSSASTKGPSVFPLA | AAPSVFIFPPSDEQLKSGTASVVCLLNN |
| | PCSRSTSESTAALGCLVKDYFPEPVT | FYPREAKVQWKVDNALQSGNSQESVT |
| | VSWNSGALTSGVHTFPAVLQSSGLY | EQDSKDSTYSLSSTLTLSKADYEKHKV |
| | SLSSVVTVPSSSLGTKTYTCNVDHKP | YACEVTHQGLSSPVTKSFNRGEC (SEQ |
| | SNTKVDKRVESKYGPPCPPCPAPEFL | ID NO: 140) |
| | GGPSVFLFPPKPKDTLMISRTPEVTCV | |
| | VVDVSQEDPEVQFNWYVDGVEVHN | |
| | AKTKPREEQFNSTYRVVSVLTVLHQ | |
| | DWLNGKEYKCKVSNKGLPSSIEKTIS | |
| | KAKGQPREPQVYTLPPSQEEMTKNQ | |
| | VSLTCLVKGFYPSDIAVEWESNGQPE | |
| | NNYKTTPPVLDSDGSFFLYSRLTVDK | |
| | SRWQEGNVFSCSVMHEALHNHYTQ | |
| | KSLSLSLGK (SEQ ID NO: 139) | |
| LK-RB1- | DVQLQESGPGLEKPSQSLSLTCSVTG | DIVLTQSPATLSVTPGDSVSLSCRASQS |
| 36 | YSITRGYQWNWIRQFPGNKLEWMG | ISNNLHWYQQKSHESPRLLIKYASQSIS |
| | YISYDGSNNYNPSLKNRISIIRDTSKN | GIPSRFSGSGSGTDFTLSINSVETEDFG |
| | QFFLKLNSVTTEDTATYYCARRSYNS | MYFCQQSNSWPLTFGAGTKLELKRTV |
| | IDYWGQGTTLTVSSASTKGPSVFPLA | AAPSVFIFPPSDEQLKSGTASVVCLLNN |
| | PCSRSTSESTAALGCLVKDYFPEPVT | FYPREAKVQWKVDNALQSGNSQESVT |
| | VSWNSGALTSGVHTFPAVLQSSGLY | EQDSKDSTYSLSSTLTLSKADYEKHKV |
| | SLSSVVTVPSSSLGTKTYTCNVDHKP | YACEVTHQGLSSPVTKSFNRGEC (SEQ |
| | SNTKVDKRVESKYGPPCPPCPAPEFL | ID NO: 142) |
| | GGPSVFLFPPKPKDTLMISRTPEVTCV | |
| | VVDVSQEDPEVQFNWYVDGVEVHN | |
| | AKTKPREEQFNSTYRVVSVLTVLHQ | |
| | DWLNGKEYKCKVSNKGLPSSIEKTIS | |
| | KAKGQPREPQVYTLPPSQEEMTKNQ | |
| | VSLTCLVKGFYPSDIAVEWESNGQPE | |
| | NNYKTTPPVLDSDGSFFLYSRLTVDK | |
| | SRWQEGNVFSCSVMHEALHNHYTQ | |
| | KSLSLSLGK (SEQ ID NO: 141) | |
| LK-RB1- | QVQLQQPGAELVKPGASVKLSCKAS | DILLTQSPAILSVSPGERVSFSCRASQSI |
| 37 | GYTFTSYWMHWVKQRPGRGPEWIG | GTSIHWYQQRRNGSPRLLIKYTSESISG |
| | RIDPNSGYARYNEKFKNKATLTADK | IPSRFSGSGSGTDFTLSINSVESEDIADY |
| | PSSTADMHLSSLTSEDSAVYYCTRSFI | YCQQSNSWPHTFGGGTKLELKRTVAA |
| | TTAEWFFDVWGTGTTVTVSSASTKG | PSVFIFPPSDEQLKSGTASVVCLLNNFY |
| | PSVFPLAPCSRSTSESTAALGCLVKD | PREAKVQWKVDNALQSGNSQESVTEQ |
| | YFPEPVTVSWNSGALTSGVHTFPAVL | DSKDSTYSLSSTLTLSKADYEKHKVYA |
| | QSSGLYSLSSVVTVPSSSLGTKTYTC | CEVTHQGLSSPVTKSFNRGEC (SEQ ID |
| | NVDHKPSNTKVDKRVESKYGPPCPP | NO: 144) |
| | CPAPEFLGGPSVFLFPPKPKDTLMISR | |
| | TPEVTCVVVDVSQEDPEVQFNWYVD | |
| | GVEVHNAKTKPREEQFNSTYRVVSV | |
| | LTVLHQDWLNGKEYKCKVSNKGLP | |
| | SSIEKTISKAKGQPREPQVYTLPPSQE | |
| | EMTKNQVSLTCLVKGFYPSDIAVEW | |
| | ESNGQPENNYKTTPPVLDSDGSFFLY | |
| | SRLTVDKSRWQEGNVFSCSVMHEAL | |
| | HNHYTQKSLSLSLGK (SEQ ID NO: | |
| | 143) | |
| LK-RB1- | DVQLQESGPGLVQPSQSLSLTCSVTG | EIVLTQSPATLSVTPGDRVSLSCRASQN |
| 38 | YSITSGYQWNWIRQLPGNKLEWMA | ISNYLHWYQQKSHGSPRLIIKYASQSIS |
| | YISFDGSNNYNPSLKNRISITRDTSKN | GIPSRFSGSGSGTDFTLSINSVETEDFG |
| | QFFLKLKSVTTEDTATYYCARRDYD | MYFCQQSNSWPLTFGAGTKLELKRTV |
| | GVAYWGQGTLVTVSAASTKGPSVFP | AAPSVFIFPPSDEQLKSGTASVVCLLNN |
| | LAPCSRSTSESTAALGCLVKDYFPEP | FYPREAKVQWKVDNALQSGNSQESVT |
| | VTVSWNSGALTSGVHTFPAVLQSSG | EQDSKDSTYSLSSTLTLSKADYEKHKV |
| | LYSLSSVVTVPSSSLGTKTYTCNVDH | YACEVTHQGLSSPVTKSFNRGEC (SEQ |
| | KPSNTKVDKRVESKYGPPCPPCPAPE | ID NO: 146) |
| | FLGGPSVFLFPPKPKDTLMISRTPEVT | |
| | CVVVDVSQEDPEVQFNWYVDGVEV | |
| | HNAKTKPREEQFNSTYRVVSVLTVL | |
| | HQDWLNGKEYKCKVSNKGLPSSIEK | |
| | TISKAKGQPREPQVYTLPPSQEEMTK | |
| | NQVSLTCLVKGFYPSDIAVEWESNG | |
| | QPENNYKTTPPVLDSDGSFFLYSRLT | |
| | VDKSRWQEGNVFSCSVMHEALHNH | |
| | YTQKSLSLSLGK (SEQ ID NO: 145) | |
| LK-RB1- | EVQLQQSGAELVRPGASVKLSCTAS | QIVLTQSPAIMSASPGEKVTITCSASSS |
| 39 | GFNIKDDYVHWVNQRPEQGLEWIG | VNYMHWFQQKPGSSPKLWIYSTANLA |
| | WIDPENGDTGYASKFQDKATITADTS | SGVPARFSGSGSGTSYSLTISRMEAED |
| | SNTAYLHLSSLTSEDTAVYYCTTTR | AATYYCQQRSSYPPTWTFGGGTKLEIK |
| | WTGSYWGQGTTLTVSSASTKGPSVF | RTVAAPSVFIFPPSDEQLKSGTASVVCL |
| | PLAPCSRSTSESTAALGCLVKDYFPE | LNNFYPREAKVQWKVDNALQSGNSQ |
| | PVTVSWNSGALTSGVHTFPAVLQSS | ESVTEQDSKDSTYSLSSTLTLSKADYE |
| | GLYSLSSVVTVPSSSLGTKTYTCNVD | KHKVYACEVTHQGLSSPVTKSFNRGE |
| | HKPSNTKVDKRVESKYGPPCPPCPAP | C (SEQ ID NO: 148) |
| | EFLGGPSVFLFPPKPKDTLMISRTPEV | |
| | TCVVVDVSQEDPEVQFNWYVDGVE | |
| | VHNAKTKPREEQFNSTYRVVSVLTV | |
| | LHQDWLNGKEYKCKVSNKGLPSSIE | |
| | KTISKAKGQPREPQVYTLPPSQEEMT | |
| | KNQVSLTCLVKGFYPSDIAVEWESN | |
| | GQPENNYKTTPPVLDSDGSFFLYSRL | |
| | TVDKSRWQEGNVFSCSVMHEALHN | |
| | HYTQKSLSLSLGK (SEQ ID NO: 147) | |
| LK-RB1- | DVQLQESGPGLVQPSQSLSLTCSVTG | DVLMTQTPLSLPVSLGDQASISCRSSQS |
| 40 | YSITSGYQWNWIRQLPGNKLEWMA | IVHSNGNTYLEWYLQKPGQSPKLLIYK |
| | YISFDGSNNYNPSLKNRISITRDTSKN | VSNRFSGVPDRFSGSGSGTDFTLKISRV |
| | QFFLKLKSVTTEDTATYYCARRDYD | EAEDLGVYYCFQGSHVPWTFGGGTKL |
| | GVAYWGQGTLVTVSAASTKGPSVFP | EIKRTVAAPSVFIFPPSDEQLKSGTASV |
| | LAPCSRSTSESTAALGCLVKDYFPEP | VCLLNNFYPREAKVQWKVDNALQSG |
| | VTVSWNSGALTSGVHTFPAVLQSSG | NSQESVTEQDSKDSTYSLSSTLTLSKA |
| | LYSLSSVVTVPSSSLGTKTYTCNVDH | DYEKHKVYACEVTHQGLSSPVTKSFN |
| | KPSNTKVDKRVESKYGPPCPPCPAPE | RGEC (SEQ ID NO: 150) |
| | FLGGPSVFLFPPKPKDTLMISRTPEVT | |
| | CVVVDVSQEDPEVQFNWYVDGVEV | |
| | HNAKTKPREEQFNSTYRVVSVLTVL | |
| | HQDWLNGKEYKCKVSNKGLPSSIEK | |
| | TISKAKGQPREPQVYTLPPSQEEMTK | |
| | NQVSLTCLVKGFYPSDIAVEWESNG | |
| | QPENNYKTTPPVLDSDGSFFLYSRLT | |
| | VDKSRWQEGNVFSCSVMHEALHNH | |
| | YTQKSLSLSLGK (SEQ ID NO: 149) | |
| LK-RB1- | DVQLQESGPGLVQPSQSLSLTCSVTG | EIVLTQSPATLSVTPGDRVSLSCRASQN |
| 41 | YSITSGYQWNWIRQLPGNKLEWMA | ISNYLHWYQQKSHGSPRLIIKYASQSIS |
| | YISFDGSNNYNPSLKNRISITRDTSKN | GIPSRFSGSGSGTDFTLSINSVETEDFG |
| | QFFLKLKSVTTEDTATYYCARRDYD | MYFCQQSNSWPLTFGAGTKLELKRTV |
| | GVAYWGQGTLVTVSAASTKGPSVFP | AAPSVFIFPPSDEQLKSGTASVVCLLNN |
| | LAPCSRSTSESTAALGCLVKDYFPEP | FYPREAKVQWKVDNALQSGNSQESVT |
| | VTVSWNSGALTSGVHTFPAVLQSSG | EQDSKDSTYSLSSTLTLSKADYEKHKV |
| | LYSLSSVVTVPSSSLGTKTYTCNVDH | YACEVTHQGLSSPVTKSFNRGEC (SEQ |
| | KPSNTKVDKRVESKYGPPCPPCPAPE | ID NO: 152) |
| | FLGGPSVFLFPPKPKDTLMISRTPEVT | |
| | CVVVDVSQEDPEVQFNWYVDGVEV | |
| | HNAKTKPREEQFNSTYRVVSVLTVL | |
| | HQDWLNGKEYKCKVSNKGLPSSIEK | |
| | TISKAKGQPREPQVYTLPPSQEEMTK | |
| | NQVSLTCLVKGFYPSDIAVEWESNG | |
| | QPENNYKTTPPVLDSDGSFFLYSRLT | |
| | VDKSRWQEGNVFSCSVMHEALHNH | |
| | YTQKSLSLSLGK (SEQ ID NO: 151) | |
| LK-RB1- | QVQLQQPGAELVKPGTSVKLSCKAS | DIVMTQSQKFMSTSVGDRVSVTCKAS |
| 42 | GYSFISYWVYWVKQRPGQGLEWIG | QNVGTNVAWYQQKPGQSPKALIYSAS |
| | MIHPNSGSTNFNEKFRYKATLTVDKS | YRFSGVPDRFTGSGSGTDFTLTIGNVQ |
| | SSTAYIQLSSLTSEDSAVYYCGRSDD | SEDLAEYFCHQYKNYPLTFGGGTKLEI |
| | GYPYHAMDSWGQGTSVTVSSASTK | KRTVAAPSVFIFPPSDEQLKSGTASVVC |
| | GPSVFPLAPCSRSTSESTAALGCLVK | LLNNFYPREAKVQWKVDNALQSGNS |
| | DYFPEPVTVSWNSGALTSGVHTFPA | QESVTEQDSKDSTYSLSSTLTLSKADY |
| | VLQSSGLYSLSSVVTVPSSSLGTKTY | EKHKVYACEVTHQGLSSPVTKSFNRG |
| | TCNVDHKPSNTKVDKRVESKYGPPC | EC (SEQ ID NO: 154) |
| | PPCPAPEFLGGPSVFLFPPKPKDTLMI | |
| | SRTPEVTCVVVDVSQEDPEVQFNWY | |
| | VDGVEVHNAKTKPREEQFNSTYRVV | |
| | SVLTVLHQDWLNGKEYKCKVSNKG | |
| | LPSSIEKTISKAKGQPREPQVYTLPPS | |
| | QEEMTKNQVSLTCLVKGFYPSDIAV | |
| | EWESNGQPENNYKTTPPVLDSDGSFF | |
| | LYSRLTVDKSRWQEGNVFSCSVMHE | |
| | ALHNHYTQKSLSLSLGK (SEQ ID | |
| | NO: 153) | |
| LK-RB1- | EFQLQQSGPELMKPGASVKISCKASG | DIQMTQSPGALSVSVGETVTITCRASE |
| 43 | YSFTDYNMNWVKQSNGKSLEWIGII | NINSNLAWYQQKQGKSPQLLVYAATN |
| | DPNYGTTTYNQKFKGRATLTVDQSS | LADGVPSRFSGSGSGTQYSLKINSLQSE |
| | NTAYMQLNSLTSEDSAVYYCAREGR | DFGSYHCQHFWGTPFTFGSGTKLEIKR |
| | YSWFAYWGQGTLVTVSAASTKGPS | TVAAPSVFIFPPSDEQLKSGTASVVCLL |
| | VFPLAPCSRSTSESTAALGCLVKDYF | NNFYPREAKVQWKVDNALQSGNSQES |
| | PEPVTVSWNSGALTSGVHTFPAVLQS | VTEQDSKDSTYSLSSTLTLSKADYEKH |
| | SGLYSLSSVVTVPSSSLGTKTYTCNV | KVYACEVTHQGLSSPVTKSFNRGEC |
| | DHKPSNTKVDKRVESKYGPPCPPCP | (SEQ ID NO: 156) |
| | APEFLGGPSVFLFPPKPKDTLMISRTP | |
| | EVTCVVVDVSQEDPEVQFNWYVDG | |
| | VEVHNAKTKPREEQFNSTYRVVSVL | |
| | TVLHQDWLNGKEYKCKVSNKGLPSS | |
| | IEKTISKAKGQPREPQVYTLPPSQEE | |
| | MTKNQVSLTCLVKGFYPSDIAVEWE | |
| | SNGQPENNYKTTPPVLDSDGSFFLYS | |
| | RLTVDKSRWQEGNVFSCSVMHEAL | |
| | HNHYTQKSLSLSLGK (SEQ ID NO: | |
| | 155) | |
| LK-RB1- | QVQLQQSGTELAKPGASVKLSCKAS | DILLTQSPAILSVSPGERVSFSCRASQSI |
| 44 | GYTFTNYWMHWIKQRPGQGLEWIG | GRSIHWFQQRINGSPSLLIKYASESISGI |
| | YVHPTSDYTKYNQKFKDKATLTADK | PSRFSGGGSGTDFTLSISNVESEDIADY |
| | SSSTAYMQLNSLTYEDSAVYYCTRSS | YCQQSNNWPFTFGSGTKLEIKRTVAAP |
| | LYDTNYVFDSWGQGTTLTVSSASTK | SVFIFPPSDEQLKSGTASVVCLLNNFYP |
| | GPSVFPLAPCSRSTSESTAALGCLVK | REAKVQWKVDNALQSGNSQESVTEQ |
| | DYFPEPVTVSWNSGALTSGVHTFPA | DSKDSTYSLSSTLTLSKADYEKHKVYA |
| | VLQSSGLYSLSSVVTVPSSSLGTKTY | CEVTHQGLSSPVTKSFNRGEC (SEQ ID |
| | TCNVDHKPSNTKVDKRVESKYGPPC | NO: 158) |
| | PPCPAPEFLGGPSVFLFPPKPKDTLMI | |
| | SRTPEVTCVVVDVSQEDPEVQFNWY | |
| | VDGVEVHNAKTKPREEQFNSTYRVV | |
| | SVLTVLHQDWLNGKEYKCKVSNKG | |
| | LPSSIEKTISKAKGQPREPQVYTLPPS | |
| | QEEMTKNQVSLTCLVKGFYPSDIAV | |
| | EWESNGQPENNYKTTPPVLDSDGSFF | |
| | LYSRLTVDKSRWQEGNVFSCSVMHE | |
| | ALHNHYTQKSLSLSLGK (SEQ ID | |
| | NO: 157) | |
| LK-RB1- | QVQLQQSGTELARPGASVKLSCKAS | DILLTQSPAILSVSPGERVSFSCRASQSI |
| 45 | GYTFTNFWMHWIKQRPGQGLEWIG | GRSIHWYQQRTNGSPRLLIKYASESISG |
| | YVNPTSDYTKYNQKFKDKATLTADK | IPSRFSGGGSGTDFTLSISNVESEDIADY |
| | SSSTAYMQLNSLTYEDSAVYYCTRSS | YCQQSNNWPFTFGSGTKLEIKRTVAAP |
| | LYDTNYVFDSWGQGTTLTVSSASTK | SVFIFPPSDEQLKSGTASVVCLLNNFYP |
| | GPSVFPLAPCSRSTSESTAALGCLVK | REAKVQWKVDNALQSGNSQESVTEQ |
| | DYFPEPVTVSWNSGALTSGVHTFPA | DSKDSTYSLSSTLTLSKADYEKHKVYA |
| | VLQSSGLYSLSSVVTVPSSSLGTKTY | CEVTHQGLSSPVTKSFNRGEC (SEQ ID |
| | TCNVDHKPSNTKVDKRVESKYGPPC | NO: 160) |
| | PPCPAPEFLGGPSVFLFPPKPKDTLMI | |
| | SRTPEVTCVVVDVSQEDPEVQFNWY | |
| | VDGVEVHNAKTKPREEQFNSTYRVV | |
| | SVLTVLHQDWLNGKEYKCKVSNKG | |
| | LPSSIEKTISKAKGQPREPQVYTLPPS | |
| | QEEMTKNQVSLTCLVKGFYPSDIAV | |
| | EWESNGQPENNYKTTPPVLDSDGSFF | |
| | LYSRLTVDKSRWQEGNVFSCSVMHE | |
| | ALHNHYTQKSLSLSLGK (SEQ ID | |
| | NO: 159) | |
| LK-RB1- | EVQLQQSGPELVKPGASVKISCKTSG | DIVLTQSPTSLAVSLGQRAIISCKASQS |
| 46 | YTFTDYYMNWVKQSHGKSLEWIGDI | VSFAGTSLMHWYQQKPGQQPKLLIYR |
| | YPINGDISYNQKFKGKATLTVDKSSS | ASNLKAGVPTRFSGSGSRTDFTLNIHP |
| | TVYMEIRSLTYEDSAVYYCARLAQT | VEEDDAATYYCQQSREYPMYTFGGGT |
| | TSGGFDYWGQGTTLTVSSASTKGPS | KLEIKRTVAAPSVFIFPPSDEQLKSGTA |
| | VFPLAPCSRSTSESTAALGCLVKDYF | SVVCLLNNFYPREAKVQWKVDNALQS |
| | PEPVTVSWNSGALTSGVHTFPAVLQS | GNSQESVTEQDSKDSTYSLSSTLTLSK |
| | SGLYSLSSVVTVPSSSLGTKTYTCNV | ADYEKHKVYACEVTHQGLSSPVTKSF |
| | DHKPSNTKVDKRVESKYGPPCPPCP | NRGEC (SEQ ID NO: 162) |
| | APEFLGGPSVFLFPPKPKDTLMISRTP | |
| | EVTCVVVDVSQEDPEVQFNWYVDG | |
| | VEVHNAKTKPREEQFNSTYRVVSVL | |
| | TVLHQDWLNGKEYKCKVSNKGLPSS | |
| | IEKTISKAKGQPREPQVYTLPPSQEE | |
| | MTKNQVSLTCLVKGFYPSDIAVEWE | |
| | SNGQPENNYKTTPPVLDSDGSFFLYS | |
| | RLTVDKSRWQEGNVFSCSVMHEAL | |
| | HNHYTQKSLSLSLGK (SEQ ID NO: | |
| | 161) | |
| LK-RB1- | QIQLVQSGPELKKPGETVKISCKASG | DIQMTQTTSSLSASLGDRVTISCRASQD |
| 47 | YTFTTYGMSWVKQAPGKGLKWMG | ISNYLNWYQQKPDGTVKLLIYYTSRIH |
| | WINTYSGVPTYTDDFKGRFAFSVETS | SGVPSRFSGSGSGTDFSLTISNLDQEDI |
| | ASTAYLQINNLKNEDTATYFCARRE | ATYFCQQGHTLPWTFGGGTKVEIKRT |
| | RDFYGNIFFPYWGQGSLVTVSAAST | VAAPSVFIFPPSDEQLKSGTASVVCLLN |
| | KGPSVFPLAPCSRSTSESTAALGCLV | NFYPREAKVQWKVDNALQSGNSQESV |
| | KDYFPEPVTVSWNSGALTSGVHTFP | TEQDSKDSTYSLSSTLTLSKADYEKHK |
| | AVLQSSGLYSLSSVVTVPSSSLGTKT | VYACEVTHQGLSSPVTKSFNRGEC |
| | YTCNVDHKPSNTKVDKRVESKYGPP | (SEQ ID NO: 164) |
| | CPPCPAPEFLGGPSVFLFPPKPKDTLM | |
| | ISRTPEVTCVVVDVSQEDPEVQFNW | |
| | YVDGVEVHNAKTKPREEQFNSTYRV | |
| | VSVLTVLHQDWLNGKEYKCKVSNK | |
| | GLPSSIEKTISKAKGQPREPQVYTLPP | |
| | SQEEMTKNQVSLTCLVKGFYPSDIAV | |
| | EWESNGQPENNYKTTPPVLDSDGSFF | |
| | LYSRLTVDKSRWQEGNVFSCSVMHE | |
| | ALHNHYTQKSLSLSLGK (SEQ ID | |
| | NO: 163) | |
| LK-RB1- | EVKLVESGGGLVQPGGSLKLSCAAS | DIVMTQSQKFLSTTVGDRVSITCKASQ |
| 48 | GFTFSDYYMYWVRQTPEKRLEWVA | NVGTAVAWYQQKPGQSPKILIYSASN |
| | YISNGGGISFYPDTVKGRFTISRDNAK | RYTGVPDRFTGSGSGTDFTLTISNMQS |
| | NTLYLQMSRLKSEDTAIYYCASATK | EDLADYFCQQYSSYRTFGGGTKLEIKR |
| | EVGAMDYWGQGTSVTVSSASTKGPS | TVAAPSVFIFPPSDEQLKSGTASVVCLL |
| | VFPLAPCSRSTSESTAALGCLVKDYF | NNFYPREAKVQWKVDNALQSGNSQES |
| | PEPVTVSWNSGALTSGVHTFPAVLQS | VTEQDSKDSTYSLSSTLTLSKADYEKH |
| | SGLYSLSSVVTVPSSSLGTKTYTCNV | KVYACEVTHQGLSSPVTKSFNRGEC |
| | DHKPSNTKVDKRVESKYGPPCPPCP | (SEQ ID NO: 166) |
| | APEFLGGPSVFLFPPKPKDTLMISRTP | |
| | EVTCVVVDVSQEDPEVQFNWYVDG | |
| | VEVHNAKTKPREEQFNSTYRVVSVL | |
| | TVLHQDWLNGKEYKCKVSNKGLPSS | |
| | IEKTISKAKGQPREPQVYTLPPSQEE | |
| | MTKNQVSLTCLVKGFYPSDIAVEWE | |
| | SNGQPENNYKTTPPVLDSDGSFFLYS | |
| | RLTVDKSRWQEGNVFSCSVMHEAL | |
| | HNHYTQKSLSLSLGK (SEQ ID NO: | |
| | 165) | |
| LK-RB1- | QIQLVQSGPELKKPGETVKISCKASG | DIVMTQSQKFMSATVGDRVSITCKASQ |
| 49 | YTFTTYGMSWVKQAPGKGLKWMG | NVGTAVAWYQQKPGQSPKLLIYSPSN |
| | WINTYSGVPTYADDFKGRFAFSLETS | RYTGVPDRFTGSGSGTHFTLTINNMQS |
| | GSTAYLQISNLKNEDTATYFCSRLFR | EDLADYFCQQYSTYPLTFGAGTKLELN |
| | SWDSYAMDYWGQGTSVTVSSASTK | RTVAAPSVFIFPPSDEQLKSGTASVVCL |
| | GPSVFPLAPCSRSTSESTAALGCLVK | LNNFYPREAKVQWKVDNALQSGNSQ |
| | DYFPEPVTVSWNSGALTSGVHTFPA | ESVTEQDSKDSTYSLSSTLTLSKADYE |
| | VLQSSGLYSLSSVVTVPSSSLGTKTY | KHKVYACEVTHQGLSSPVTKSFNRGE |
| | TCNVDHKPSNTKVDKRVESKYGPPC | C (SEQ ID NO: 168) |
| | PPCPAPEFLGGPSVFLFPPKPKDTLMI | |
| | SRTPEVTCVVVDVSQEDPEVQFNWY | |
| | VDGVEVHNAKTKPREEQFNSTYRVV | |
| | SVLTVLHQDWLNGKEYKCKVSNKG | |
| | LPSSIEKTISKAKGQPREPQVYTLPPS | |
| | QEEMTKNQVSLTCLVKGFYPSDIAV | |
| | EWESNGQPENNYKTTPPVLDSDGSFF | |
| | LYSRLTVDKSRWQEGNVFSCSVMHE | |
| | ALHNHYTQKSLSLSLGK (SEQ ID | |
| | NO: 167) | |
| LK-RB1- | QVQLKQSGPGLVQPSQSLSITCTVSG | QIVLSQSPAILSAFPGEKVTMTCRASSS |
| 50 | FSLTSYGVHWVRQSPGKGLEWLGVI | VSFMHWYQQKPGSSPKPWIYATSNLA |
| | WRGGNTDYNAPFVSRLSITKDNSQS | SGVPARFSGSGSGTSYSLTISRVEAEDA |
| | QVFFKMNSLEADDTAIYYCAKNSLD | ATYYCQQWSSNPLTFGAGTKLELKRT |
| | VYSSWYFDVWGTGTTVTVSSASTKG | VAAPSVFIFPPSDEQLKSGTASVVCLLN |
| | PSVFPLAPCSRSTSESTAALGCLVKD | NFYPREAKVQWKVDNALQSGNSQESV |
| | YFPEPVTVSWNSGALTSGVHTFPAVL | TEQDSKDSTYSLSSTLTLSKADYEKHK |
| | QSSGLYSLSSVVTVPSSSLGTKTYTC | VYACEVTHQGLSSPVTKSFNRGEC |
| | NVDHKPSNTKVDKRVESKYGPPCPP | (SEQ ID NO: 170) |
| | CPAPEFLGGPSVFLFPPKPKDTLMISR | |
| | TPEVTCVVVDVSQEDPEVQFNWYVD | |
| | GVEVHNAKTKPREEQFNSTYRVVSV | |
| | LTVLHQDWLNGKEYKCKVSNKGLP | |
| | SSIEKTISKAKGQPREPQVYTLPPSQE | |
| | EMTKNQVSLTCLVKGFYPSDIAVEW | |
| | ESNGQPENNYKTTPPVLDSDGSFFLY | |
| | SRLTVDKSRWQEGNVFSCSVMHEAL | |
| | HNHYTQKSLSLSLGK (SEQ ID NO: | |
| | 169) | |
| LK-RB1- | QVQIQQSGAELVKPGASVKLSCKAS | ENVLTQSPAIMSASLGEKVTMSCRASS |
| 51 | GYTFTEFPIHWIKQRSGQGLEWIGWF | SVNYMYWYQQKSDASPKIWIYYTSNL |
| | YPGSDNMNYNEKFKDKATLTADKSS | APGVPARFSGSGSGNSYSLTISSMEGE |
| | STVYMELSRLTSEDSAVYFCARHEN | DAATYYCQQFTSSPFTFGTGTKLEIKR |
| | AYFYAMDYWGQGTSVTVSSASTKG | TVAAPSVFIFPPSDEQLKSGTASVVCLL |
| | PSVFPLAPCSRSTSESTAALGCLVKD | NNFYPREAKVQWKVDNALQSGNSQES |
| | YFPEPVTVSWNSGALTSGVHTFPAVL | VTEQDSKDSTYSLSSTLTLSKADYEKH |
| | QSSGLYSLSSVVTVPSSSLGTKTYTC | KVYACEVTHQGLSSPVTKSFNRGEC |
| | NVDHKPSNTKVDKRVESKYGPPCPP | (SEQ ID NO: 172) |
| | CPAPEFLGGPSVFLFPPKPKDTLMISR | |
| | TPEVTCVVVDVSQEDPEVQFNWYVD | |
| | GVEVHNAKTKPREEQFNSTYRVVSV | |
| | LTVLHQDWLNGKEYKCKVSNKGLP | |
| | SSIEKTISKAKGQPREPQVYTLPPSQE | |
| | EMTKNQVSLTCLVKGFYPSDIAVEW | |
| | ESNGQPENNYKTTPPVLDSDGSFFLY | |
| | SRLTVDKSRWQEGNVFSCSVMHEAL | |
| | HNHYTQKSLSLSLGK (SEQ ID NO: | |
| | 171) | |
| LK-RB1- | EVKLVESGGGLVQPGGSLKLSCAAS | DIVMTQSQKFMSTTVGDRVSITCKASQ |
| 52 | GFIFSDYYMYWVRQTPEKRLEWVA | NVGTAVAWYQQKPGQSPKLLIYAASN |
| | YISNGGDITYYPDTVKGRFTISRDIAR | RYTGVPDRFTGSGSGTDFTLTISNMQS |
| | NTLFLQMGRLKSEDTAMYYCASATT | EDLADYFCQQYRSYRTFGGGTNLEIKR |
| | EVGAMDHWGQGTSVTVSSASTKGPS | TVAAPSVFIFPPSDEQLKSGTASVVCLL |
| | VFPLAPCSRSTSESTAALGCLVKDYF | NNFYPREAKVQWKVDNALQSGNSQES |
| | PEPVTVSWNSGALTSGVHTFPAVLQS | VTEQDSKDSTYSLSSTLTLSKADYEKH |
| | SGLYSLSSVVTVPSSSLGTKTYTCNV | KVYACEVTHQGLSSPVTKSFNRGEC |
| | DHKPSNTKVDKRVESKYGPPCPPCP | (SEQ ID NO: 174) |
| | APEFLGGPSVFLFPPKPKDTLMISRTP | |
| | EVTCVVVDVSQEDPEVQFNWYVDG | |
| | VEVHNAKTKPREEQFNSTYRVVSVL | |
| | TVLHQDWLNGKEYKCKVSNKGLPSS | |
| | IEKTISKAKGQPREPQVYTLPPSQEE | |
| | MTKNQVSLTCLVKGFYPSDIAVEWE | |
| | SNGQPENNYKTTPPVLDSDGSFFLYS | |
| | RLTVDKSRWQEGNVFSCSVMHEAL | |
| | HNHYTQKSLSLSLGK (SEQ ID NO: | |
| | 173) | |
| LK-RB1- | QIQLVQSGPELKKPGETVKISCKASG | DIVMTQSQKFMSTTVGDRVSITCKASQ |
| 53 | YTFTTYGMSWVNQAPGKGLKWMG | NVGTAVAWYQQKPGQSPKLLIYSASN |
| | WINTNYGVPTYADDFKGRFAFSLETS | RYTGVPDRFTGSDSGTDFTLTINNIQSE |
| | ASTAYLQINNLKNEDTATYFCARGT | DLADYFCQQYSSYPLTFATGTKLELKR |
| | RRRGISYFAMDYWGQGTSVTVSSAS | TVAAPSVFIFPPSDEQLKSGTASVVCLL |
| | TKGPSVFPLAPCSRSTSESTAALGCL | NNFYPREAKVQWKVDNALQSGNSQES |
| | VKDYFPEPVTVSWNSGALTSGVHTF | VTEQDSKDSTYSLSSTLTLSKADYEKH |
| | PAVLQSSGLYSLSSVVTVPSSSLGTK | KVYACEVTHQGLSSPVTKSFNRGEC |
| | TYTCNVDHKPSNTKVDKRVESKYGP | (SEQ ID NO: 176) |
| | PCPPCPAPEFLGGPSVFLFPPKPKDTL | |
| | MISRTPEVTCVVVDVSQEDPEVQFN | |
| | WYVDGVEVHNAKTKPREEQFNSTY | |
| | RVVSVLTVLHQDWLNGKEYKCKVS | |
| | NKGLPSSIEKTISKAKGQPREPQVYTL | |
| | PPSQEEMTKNQVSLTCLVKGFYPSDI | |
| | AVEWESNGQPENNYKTTPPVLDSDG | |
| | SFFLYSRLTVDKSRWQEGNVFSCSV | |
| | MHEALHNHYTQKSLSLSLGK (SEQ | |
| | ID NO: 175) | |
| LK-RB1- | DVQLQESGPGLVKPSQSLSLTCSVTG | ENVLTQSPAIMSASLGEKVTMSCRASS |
| 54 | YSITSGFYWNWIRQFPGNKLEWMGH | SVNYMYWYQQKSDASPKLWIYYTSN |
| | ISYDGTNNYNPSLKNRISITRDTSKNL | LAPGVPVRFSGSGSGNSYSLTISSMEGE |
| | FFLKLNSVTTEDTATFHCARGGYGG | DAATYYCQQFTSSPWTFGGGTKLEIKR |
| | SYGFDYWGQGTTLTVSSASTKGPSV | TVAAPSVFIFPPSDEQLKSGTASVVCLL |
| | FPLAPCSRSTSESTAALGCLVKDYFP | NNFYPREAKVQWKVDNALQSGNSQES |
| | EPVTVSWNSGALTSGVHTFPAVLQSS | VTEQDSKDSTYSLSSTLTLSKADYEKH |
| | GLYSLSSVVTVPSSSLGTKTYTCNVD | KVYACEVTHQGLSSPVTKSFNRGEC |
| | HKPSNTKVDKRVESKYGPPCPPCPAP | (SEQ ID NO: 178) |
| | EFLGGPSVFLFPPKPKDTLMISRTPEV | |
| | TCVVVDVSQEDPEVQFNWYVDGVE | |
| | VHNAKTKPREEQFNSTYRVVSVLTV | |
| | LHQDWLNGKEYKCKVSNKGLPSSIE | |
| | KTISKAKGQPREPQVYTLPPSQEEMT | |
| | KNQVSLTCLVKGFYPSDIAVEWESN | |
| | GQPENNYKTTPPVLDSDGSFFLYSRL | |
| | TVDKSRWQEGNVFSCSVMHEALHN | |
| | HYTQKSLSLSLGK (SEQ ID NO: 177) | |
| LK-RB1- | EVKLVESGGGLVQPGGSLKLSCAAS | DIVMTQSQKFMSTTVGDRVSITCKASQ |
| 55 | GFTFSDYYMYWVRQTPEKRLEWVA | NVGTAVAWYQQKPGKSPKLLIYAASN |
| | YISNGGDITFYPDTVKGRFTISRDNAR | RYTGVPDRFTGSGSGTDFTLTISNMQS |
| | STLYLQMSRLKSEDTAIYYCASATTE | EDLADYFCQQYSSYRTFGGGTNLEIKR |
| | VGAMDHWGQGTSVTVSSASTKGPS | TVAAPSVFIFPPSDEQLKSGTASVVCLL |
| | VFPLAPCSRSTSESTAALGCLVKDYF | NNFYPREAKVQWKVDNALQSGNSQES |
| | PEPVTVSWNSGALTSGVHTFPAVLQS | VTEQDSKDSTYSLSSTLTLSKADYEKH |
| | SGLYSLSSVVTVPSSSLGTKTYTCNV | KVYACEVTHQGLSSPVTKSFNRGEC |
| | DHKPSNTKVDKRVESKYGPPCPPCP | (SEQ ID NO: 180) |
| | APEFLGGPSVFLFPPKPKDTLMISRTP | |
| | EVTCVVVDVSQEDPEVQFNWYVDG | |
| | VEVHNAKTKPREEQFNSTYRVVSVL | |
| | TVLHQDWLNGKEYKCKVSNKGLPSS | |
| | IEKTISKAKGQPREPQVYTLPPSQEE | |
| | MTKNQVSLTCLVKGFYPSDIAVEWE | |
| | SNGQPENNYKTTPPVLDSDGSFFLYS | |
| | RLTVDKSRWQEGNVFSCSVMHEAL | |
| | HNHYTQKSLSLSLGK (SEQ ID NO: | |
| | 179) | |
| LK-RB1- | QVQLKQSGPGLVQPSQSLSITCSVSG | QIVLSQSPAILSASPGEKVTMTCRASSS |
| 56 | FSLTNYGVHWVRQSPGKGLEWLGVI | VSFMHWYQQKPGSSPKPWIYATSNLA |
| | WRGGSTDNNAAFMSRLSITKDNSKS | SGVPARFSGSGSGTSYSLTISRVEAEDA |
| | QVFFKMNSLQADDTAIYYCAKNSDF | ATYYCQQWSRNPPTFGAGTKLELKRT |
| | YGSSWYFDVWGTGTTVTVSSASTKG | VAAPSVFIFPPSDEQLKSGTASVVCLLN |
| | PSVFPLAPCSRSTSESTAALGCLVKD | NFYPREAKVQWKVDNALQSGNSQESV |
| | YFPEPVTVSWNSGALTSGVHTFPAVL | TEQDSKDSTYSLSSTLTLSKADYEKHK |
| | QSSGLYSLSSVVTVPSSSLGTKTYTC | VYACEVTHQGLSSPVTKSFNRGEC |
| | NVDHKPSNTKVDKRVESKYGPPCPP | (SEQ ID NO: 182) |
| | CPAPEFLGGPSVFLFPPKPKDTLMISR | |
| | TPEVTCVVVDVSQEDPEVQFNWYVD | |
| | GVEVHNAKTKPREEQFNSTYRVVSV | |
| | LTVLHQDWLNGKEYKCKVSNKGLP | |
| | SSIEKTISKAKGQPREPQVYTLPPSQE | |
| | EMTKNQVSLTCLVKGFYPSDIAVEW | |
| | ESNGQPENNYKTTPPVLDSDGSFFLY | |
| | SRLTVDKSRWQEGNVFSCSVMHEAL | |
| | HNHYTQKSLSLSLGK (SEQ ID NO: | |
| | 181) | |
| LK-RB1- | EVKLVESGGGLVQPGGSLKLSCAAS | DIVMTQSQKFMSTTVGDRVSITCKASQ |
| 57 | GFTFSDYYMYWVRQTPEKRLEWVA | NVGTAVAWYQQKPGKSPKLLIYAASN |
| | YISNGGDITFYPDTVKGRFTISRDNAR | RYTGVPDRFTGSGSGTDFTLTISNVQSE |
| | STLYLQTSRLKSEDTAIYYCASATTE | DLADYFCQQYSSYRTFGGGTSLEIKRT |
| | VGAMDHWGQGTSVTVSSASTKGPS | VAAPSVFIFPPSDEQLKSGTASVVCLLN |
| | VFPLAPCSRSTSESTAALGCLVKDYF | NFYPREAKVQWKVDNALQSGNSQESV |
| | PEPVTVSWNSGALTSGVHTFPAVLQS | TEQDSKDSTYSLSSTLTLSKADYEKHK |
| | SGLYSLSSVVTVPSSSLGTKTYTCNV | VYACEVTHQGLSSPVTKSFNRGEC |
| | DHKPSNTKVDKRVESKYGPPCPPCP | (SEQ ID NO: 184) |
| | APEFLGGPSVFLFPPKPKDTLMISRTP | |
| | EVTCVVVDVSQEDPEVQFNWYVDG | |
| | VEVHNAKTKPREEQFNSTYRVVSVL | |
| | TVLHQDWLNGKEYKCKVSNKGLPSS | |
| | IEKTISKAKGQPREPQVYTLPPSQEE | |
| | MTKNQVSLTCLVKGFYPSDIAVEWE | |
| | SNGQPENNYKTTPPVLDSDGSFFLYS | |
| | RLTVDKSRWQEGNVFSCSVMHEAL | |
| | HNHYTQKSLSLSLGK (SEQ ID NO: | |
| | 183) | |
| LK-RB1- | QVQLKQSGPGLVQPSQSLSITCTVSG | QIVLSQSPAILSASPGEKVTMTCRASSS |
| 58 | FSLTTYGVHWVRQSPGKGLEWLGVI | VSFMHWYQQKPGSSPKPWIYATSNLA |
| | WRGGSTDYNAAFMSRLSITKDNSKS | SGVPARFSGSGSGTSYSLTISRVEAEDA |
| | QVFFRMNSLQADDTARYYCAKNSL | ATYYCQQWSSNPPTFGGGTKLEIKRTV |
| | YYGSSWYFDVWGTGTTVTVSSASTK | AAPSVFIFPPSDEQLKSGTASVVCLLNN |
| | GPSVFPLAPCSRSTSESTAALGCLVK | FYPREAKVQWKVDNALQSGNSQESVT |
| | DYFPEPVTVSWNSGALTSGVHTFPA | EQDSKDSTYSLSSTLTLSKADYEKHKV |
| | VLQSSGLYSLSSVVTVPSSSLGTKTY | YACEVTHQGLSSPVTKSFNRGEC (SEQ |
| | TCNVDHKPSNTKVDKRVESKYGPPC | ID NO: 186) |
| | PPCPAPEFLGGPSVFLFPPKPKDTLMI | |
| | SRTPEVTCVVVDVSQEDPEVQFNWY | |
| | VDGVEVHNAKTKPREEQFNSTYRVV | |
| | SVLTVLHQDWLNGKEYKCKVSNKG | |
| | LPSSIEKTISKAKGQPREPQVYTLPPS | |
| | QEEMTKNQVSLTCLVKGFYPSDIAV | |
| | EWESNGQPENNYKTTPPVLDSDGSFF | |
| | LYSRLTVDKSRWQEGNVFSCSVMHE | |
| | ALHNHYTQKSLSLSLGK (SEQ ID | |
| | NO: 185) | |
| LK-RB1- | QIQLVQSGPELKKPGETVKISCKASG | DIQMTQSPASLSVSVGETVTITCRASEN |
| 59 | YTFTTYGMSWVKQAPGKGLKWMG | IYSNLVWYQQKQGKSPQLLVYTATNL |
| | WINTYSGVPTYADDFKGRFAFSLETS | ADGVPSRFSGSGSGTQFSLKINSLQSED |
| | ASTAYLQINNLKNEDTATYFCARRRI | FGSYYCQHFWGTPWTFGGGTKLEIKR |
| | YYGNKGYAMDYWGQGTSVTVSSAS | TVAAPSVFIFPPSDEQLKSGTASVVCLL |
| | TKGPSVFPLAPCSRSTSESTAALGCL | NNFYPREAKVQWKVDNALQSGNSQES |
| | VKDYFPEPVTVSWNSGALTSGVHTF | VTEQDSKDSTYSLSSTLTLSKADYEKH |
| | PAVLQSSGLYSLSSVVTVPSSSLGTK | KVYACEVTHQGLSSPVTKSFNRGEC |
| | TYTCNVDHKPSNTKVDKRVESKYGP | (SEQ ID NO: 188) |
| | PCPPCPAPEFLGGPSVFLFPPKPKDTL | |
| | MISRTPEVTCVVVDVSQEDPEVQFN | |
| | WYVDGVEVHNAKTKPREEQFNSTY | |
| | RVVSVLTVLHQDWLNGKEYKCKVS | |
| | NKGLPSSIEKTISKAKGQPREPQVYTL | |
| | PPSQEEMTKNQVSLTCLVKGFYPSDI | |
| | AVEWESNGQPENNYKTTPPVLDSDG | |
| | SFFLYSRLTVDKSRWQEGNVFSCSV | |
| | MHEALHNHYTQKSLSLSLGK (SEQ | |
| | ID NO: 187) | |
| LK-RB2-1 | QIQLVQSGPELKKPGETVKISCKASG | DIVMSQSPSSLAVSVGEKVTMSCKSSQ |
| | YTFTTYGMSWVKQAPGKGLKWMG | SLLFSSNQKNYLAWYQQKPGQSPKLLI |
| | WINTYSGVSTYADDFKGRFAFSLETS | YWASTRESGVPDRFTGRGSGTDFTLTI |
| | ASTAYLQINNLKNEDTATYFCARRY | SSVKTEDLAVYYCQQYDTYPPTFGGG |
| | DYDGYAMDYWGQGTSVTVSSASTK | TKLEIKRTVAAPSVFIFPPSDEQLKSGT |
| | GPSVFPLAPCSRSTSESTAALGCLVK | ASVVCLLNNFYPREAKVQWKVDNAL |
| | DYFPEPVTVSWNSGALTSGVHTFPA | QSGNSQESVTEQDSKDSTYSLSSTLTLS |
| | VLQSSGLYSLSSVVTVPSSSLGTKTY | KADYEKHKVYACEVTHQGLSSPVTKS |
| | TCNVDHKPSNTKVDKRVESKYGPPC | FNRGEC (SEQ ID NO: 190) |
| | PPCPAPEFLGGPSVFLFPPKPKDTLMI | |
| | SRTPEVTCVVVDVSQEDPEVQFNWY | |
| | VDGVEVHNAKTKPREEQFNSTYRVV | |
| | SVLTVLHQDWLNGKEYKCKVSNKG | |
| | LPSSIEKTISKAKGQPREPQVYTLPPS | |
| | QEEMTKNQVSLTCLVKGFYPSDIAV | |
| | EWESNGQPENNYKTTPPVLDSDGSFF | |
| | LYSRLTVDKSRWQEGNVFSCSVMHE | |
| | ALHNHYTQKSLSLSLGK (SEQ ID | |
| | NO: 189) | |
| LK-RB2-2 | DVKLEESGEGLVKPGGSLKLSCAAS | DIQMTQSPASLSVSVGETVTITCRASEN |
| | GFTFSSYAMSWVRQTPEKRLEWVAY | IYSNLAWYQQRQGKSPQLLVYAATNL |
| | ISSGGDYFHYADTVKGRFTISRDNAR | ADGVPSRFSGSGSGTQYSLKINSLQSE |
| | NTLYLQMSSLKSEDTAMYYCTREGA | DFGSYYCQQFWDNPLTFGAGTKLELK |
| | HYDSSYLAWFAHWGQGTLVTVSAA | RTVAAPSVFIFPPSDEQLKSGTASVVCL |
| | STKGPSVFPLAPCSRSTSESTAALGCL | LNNFYPREAKVQWKVDNALQSGNSQ |
| | VKDYFPEPVTVSWNSGALTSGVHTF | ESVTEQDSKDSTYSLSSTLTLSKADYE |
| | PAVLQSSGLYSLSSVVTVPSSSLGTK | KHKVYACEVTHQGLSSPVTKSFNRGE |
| | TYTCNVDHKPSNTKVDKRVESKYGP | C (SEQ ID NO: 192) |
| | PCPPCPAPEFLGGPSVFLFPPKPKDTL | |
| | MISRTPEVTCVVVDVSQEDPEVQFN | |
| | WYVDGVEVHNAKTKPREEQFNSTY | |
| | RVVSVLTVLHQDWLNGKEYKCKVS | |
| | NKGLPSSIEKTISKAKGQPREPQVYTL | |
| | PPSQEEMTKNQVSLTCLVKGFYPSDI | |
| | AVEWESNGQPENNYKTTPPVLDSDG | |
| | SFFLYSRLTVDKSRWQEGNVFSCSV | |
| | MHEALHNHYTQKSLSLSLGK (SEQ | |
| | ID NO: 191) | |
| LK-RB2-3 | QLQLQQSDAELVKPGASVKISCKVS | DIKMTQSPSSMYASLGERVTITCKASQ |
| | GYSFTDHTIHWMKQRPEQGLEWIGY | DIKSYLSWFQQKPGKSPKTLIYRANRL |
| | IYPRDGSTKENEKFTGKATLTADKSS | VDGVPSRFSGSGSGQDYSLTINSLEYE |
| | STAYMQLTSLTSEDSAVYFCTGSGFD | DMEIYYCLQYDEFPYTFGGGTKLEIKR |
| | YWGQGTSLTVSSASTKGPSVFPLAPC | TVAAPSVFIFPPSDEQLKSGTASVVCLL |
| | SRSTSESTAALGCLVKDYFPEPVTVS | NNFYPREAKVQWKVDNALQSGNSQES |
| | WNSGALTSGVHTFPAVLQSSGLYSLS | VTEQDSKDSTYSLSSTLTLSKADYEKH |
| | SVVTVPSSSLGTKTYTCNVDHKPSNT | KVYACEVTHQGLSSPVTKSFNRGEC |
| | KVDKRVESKYGPPCPPCPAPEFLGGP | (SEQ ID NO: 194) |
| | SVFLFPPKPKDTLMISRTPEVTCVVV | |
| | DVSQEDPEVQFNWYVDGVEVHNAK | |
| | TKPREEQFNSTYRVVSVLTVLHQDW | |
| | LNGKEYKCKVSNKGLPSSIEKTISKA | |
| | KGQPREPQVYTLPPSQEEMTKNQVS | |
| | LTCLVKGFYPSDIAVEWESNGQPEN | |
| | NYKTTPPVLDSDGSFFLYSRLTVDKS | |
| | RWQEGNVFSCSVMHEALHNHYTQK | |
| | SLSLSLGK (SEQ ID NO: 193) | |
| LK-RB2-4 | EVQLVESGGDLVKPGGSLKLSCAAS | DIQMTQTTSSLSASLGDRVTISCRANQ |
| | GFTFSNYGMSWVRQTPDKRLEWVA | DISNYLNWYQQKPDGTVKLLIYYTSRL |
| | TISSGGSYTFYPDSVKGRFTISRDNAK | HSGVPSRFSGSGSGTDYSLTISDLEQED |
| | NTLYLQMSSLKSEDTALYYCARDYY | IATYFCQQGNTLPWTFGGGTKLEIKRT |
| | YGKTIEGYFDYWGQGTTLTVSSAST | VAAPSVFIFPPSDEQLKSGTASVVCLLN |
| | KGPSVFPLAPCSRSTSESTAALGCLV | NFYPREAKVQWKVDNALQSGNSQESV |
| | KDYFPEPVTVSWNSGALTSGVHTFP | TEQDSKDSTYSLSSTLTLSKADYEKHK |
| | AVLQSSGLYSLSSVVTVPSSSLGTKT | VYACEVTHQGLSSPVTKSFNRGEC |
| | YTCNVDHKPSNTKVDKRVESKYGPP | (SEQ ID NO: 196) |
| | CPPCPAPEFLGGPSVFLFPPKPKDTLM | |
| | ISRTPEVTCVVVDVSQEDPEVQFNW | |
| | YVDGVEVHNAKTKPREEQFNSTYRV | |
| | VSVLTVLHQDWLNGKEYKCKVSNK | |
| | GLPSSIEKTISKAKGQPREPQVYTLPP | |
| | SQEEMTKNQVSLTCLVKGFYPSDIAV | |
| | EWESNGQPENNYKTTPPVLDSDGSFF | |
| | LYSRLTVDKSRWQEGNVFSCSVMHE | |
| | ALHNHYTQKSLSLSLGK (SEQ ID | |
| | NO: 195) | |
| LK-RB2-5 | QIQLVQSGPELKKPGETVKISCKASG | DIVMTQSPATLSVTPGDRVSLSCRASQ |
| | YTFTTYGMSWVKQAPGKGLKWMG | SISDYLHWYQQKSHESPRLLIKYASQSI |
| | WINTYSGVPTYADDFKGRFAFSLETS | SGIPSRFSGSGSGSDFTLSINSVEPEDVG |
| | VSTAYLQINNLKNEDTATYFCARRD | VYYCQNGHGFPWTFGGGTKLEIKRTV |
| | GYDGGGNFDYWGQGTTLTVSSAST | AAPSVFIFPPSDEQLKSGTASVVCLLNN |
| | KGPSVFPLAPCSRSTSESTAALGCLV | FYPREAKVQWKVDNALQSGNSQESVT |
| | KDYFPEPVTVSWNSGALTSGVHTFP | EQDSKDSTYSLSSTLTLSKADYEKHKV |
| | AVLQSSGLYSLSSVVTVPSSSLGTKT | YACEVTHQGLSSPVTKSFNRGEC (SEQ |
| | YTCNVDHKPSNTKVDKRVESKYGPP | ID NO: 198) |
| | CPPCPAPEFLGGPSVFLFPPKPKDTLM | |
| | ISRTPEVTCVVVDVSQEDPEVQFNW | |
| | YVDGVEVHNAKTKPREEQFNSTYRV | |
| | VSVLTVLHQDWLNGKEYKCKVSNK | |
| | GLPSSIEKTISKAKGQPREPQVYTLPP | |
| | SQEEMTKNQVSLTCLVKGFYPSDIAV | |
| | EWESNGQPENNYKTTPPVLDSDGSFF | |
| | LYSRLTVDKSRWQEGNVFSCSVMHE | |
| | ALHNHYTQKSLSLSLGK (SEQ ID | |
| | NO: 197) | |
| LK-RB2-6 | QIQLVQSGPELKKPGETVKISCKASG | DIVMSQSPSSLAVSVGEKVTVSCKSSQ |
| | YTFTTYGMSWVKQAPGKGLEWMG | SLLYSYNQKNYLAWYQQKPGQSPKLL |
| | WINIYSGVPTYADDFKGRFAFSLETS | IYWSSTRESGVPDRFTGSGSGTDFTLTI |
| | ASTAYLQINNLKNEDTATYFCARRG | SSVKAEDLAVYYCQQCYNYPYTFGGG |
| | DYDSFYFDYWGQGTTLTVSSASTKG | TKLEIKRTVAAPSVFIFPPSDEQLKSGT |
| | PSVFPLAPCSRSTSESTAALGCLVKD | ASVVCLLNNFYPREAKVQWKVDNAL |
| | YFPEPVTVSWNSGALTSGVHTFPAVL | QSGNSQESVTEQDSKDSTYSLSSTLTLS |
| | QSSGLYSLSSVVTVPSSSLGTKTYTC | KADYEKHKVYACEVTHQGLSSPVTKS |
| | NVDHKPSNTKVDKRVESKYGPPCPP | FNRGEC (SEQ ID NO: 200) |
| | CPAPEFLGGPSVFLFPPKPKDTLMISR | |
| | TPEVTCVVVDVSQEDPEVQFNWYVD | |
| | GVEVHNAKTKPREEQFNSTYRVVSV | |
| | LTVLHQDWLNGKEYKCKVSNKGLP | |
| | SSIEKTISKAKGQPREPQVYTLPPSQE | |
| | EMTKNQVSLTCLVKGFYPSDIAVEW | |
| | ESNGQPENNYKTTPPVLDSDGSFFLY | |
| | SRLTVDKSRWQEGNVFSCSVMHEAL | |
| | HNHYTQKSLSLSLGK (SEQ ID NO: | |
| | 199) | |
| LK-RB2-7 | DVKLVESGEGLVKPGGSLKLSCAAS | DIQMTQSPASLSVSVGETVTITCRASEN |
| | GFTFSSYAMSWVRQTPEMRLEWVA | IYSNLAWYQQKEGKSPQLLVYGATNL |
| | YISSGGDHFYYADTVKGRFTISRDNA | ADGVPSRFSGSGSGTQYSLKINSLQSE |
| | RSTLYLQMSSLKSEDTAMYYCTREG | DFGSYYCQNFWDIPLTFGAGTKLELKR |
| | ASYDSNFLAWFAYWGQGTLVTVSA | TVAAPSVFIFPPSDEQLKSGTASVVCLL |
| | ASTKGPSVFPLAPCSRSTSESTAALGC | NNFYPREAKVQWKVDNALQSGNSQES |
| | LVKDYFPEPVTVSWNSGALTSGVHT | VTEQDSKDSTYSLSSTLTLSKADYEKH |
| | FPAVLQSSGLYSLSSVVTVPSSSLGTK | KVYACEVTHQGLSSPVTKSFNRGEC |
| | TYTCNVDHKPSNTKVDKRVESKYGP | (SEQ ID NO: 202) |
| | PCPPCPAPEFLGGPSVFLFPPKPKDTL | |
| | MISRTPEVTCVVVDVSQEDPEVQFN | |
| | WYVDGVEVHNAKTKPREEQFNSTY | |
| | RVVSVLTVLHQDWLNGKEYKCKVS | |
| | NKGLPSSIEKTISKAKGQPREPQVYTL | |
| | PPSQEEMTKNQVSLTCLVKGFYPSDI | |
| | AVEWESNGQPENNYKTTPPVLDSDG | |
| | SFFLYSRLTVDKSRWQEGNVFSCSV | |
| | MHEALHNHYTQKSLSLSLGK (SEQ | |
| | ID NO: 201) | |
| LK-RB2-8 | QIQLVQSGPELKKPGETVKISCKASG | DIQMTQSPASLSASVGETVTITCRTSEN |
| | YSFASYGMGWVKQAPGKGLKWLG | IYSYLAWYQQKQGKSPQLLVYNAKSL |
| | WINTHSGVPTYADDFKGRFAFSLETS | AEGVPSRFSGSGSGTQFSLRINSLQPED |
| | ASTAYLQINNLKNEDTATYFCAHYG | FGTYYCQQHYETPLTFGAGTKLELKRT |
| | RPWFPYWGQGTLVTVSAASTKGPSV | VAAPSVFIFPPSDEQLKSGTASVVCLLN |
| | FPLAPCSRSTSESTAALGCLVKDYFP | NFYPREAKVQWKVDNALQSGNSQESV |
| | EPVTVSWNSGALTSGVHTFPAVLQSS | TEQDSKDSTYSLSSTLTLSKADYEKHK |
| | GLYSLSSVVTVPSSSLGTKTYTCNVD | VYACEVTHQGLSSPVTKSFNRGEC |
| | HKPSNTKVDKRVESKYGPPCPPCPAP | (SEQ ID NO: 204) |
| | EFLGGPSVFLFPPKPKDTLMISRTPEV | |
| | TCVVVDVSQEDPEVQFNWYVDGVE | |
| | VHNAKTKPREEQFNSTYRVVSVLTV | |
| | LHQDWLNGKEYKCKVSNKGLPSSIE | |
| | KTISKAKGQPREPQVYTLPPSQEEMT | |
| | KNQVSLTCLVKGFYPSDIAVEWESN | |
| | GQPENNYKTTPPVLDSDGSFFLYSRL | |
| | TVDKSRWQEGNVFSCSVMHEALHN | |
| | HYTQKSLSLSLGK (SEQ ID NO: 203) | |
| LK-RB2-9 | DVQLQESGPGLVKPSQSLSLTCSVTG | DIQMTQSPASLSVSVGETVTITCRASEN |
| | YSITSGYYWNWIRQFPGNKLEWMG | IYSNLAWYQQKQGKSPQLLVYGATNL |
| | YIGYDGNNNYNPSLKNRISITRDTSK | ADGVPSRFSGRGSDTQYSLKINSLQSE |
| | NQFFLKLNSLTTEDTATYYCAREGST | DFGSYYCQHFWDTPYTFGGGTKLEIK |
| | MVTSWFAYWGQGTLVTVSAASTKG | RTVAAPSVFIFPPSDEQLKSGTASVVCL |
| | PSVFPLAPCSRSTSESTAALGCLVKD | LNNFYPREAKVQWKVDNALQSGNSQ |
| | YFPEPVTVSWNSGALTSGVHTFPAVL | ESVTEQDSKDSTYSLSSTLTLSKADYE |
| | QSSGLYSLSSVVTVPSSSLGTKTYTC | KHKVYACEVTHQGLSSPVTKSFNRGE |
| | NVDHKPSNTKVDKRVESKYGPPCPP | C (SEQ ID NO: 206) |
| | CPAPEFLGGPSVFLFPPKPKDTLMISR | |
| | TPEVTCVVVDVSQEDPEVQFNWYVD | |
| | GVEVHNAKTKPREEQFNSTYRVVSV | |
| | LTVLHQDWLNGKEYKCKVSNKGLP | |
| | SSIEKTISKAKGQPREPQVYTLPPSQE | |
| | EMTKNQVSLTCLVKGFYPSDIAVEW | |
| | ESNGQPENNYKTTPPVLDSDGSFFLY | |
| | SRLTVDKSRWQEGNVFSCSVMHEAL | |
| | HNHYTQKSLSLSLGK (SEQ ID NO: | |
| | 205) | |
| LK-RB2- | EVMLVESGGDLVKPGGSLKLSCAAS | DIVMTQSQKFMSSTGGDRVTITCKASQ |
| 10 | GFTFSANTVSWLRRTPERRLEWVATI | NVDTAVAWYQQKPGQSPKLLIYSASN |
| | SGGSDNTYCPDSVKGRFTISRDNARD | RYIGVPDRFKGSGSGTDFSLTITNIQSE |
| | TLYLQMSSLTSEDTALYFCARHYYD | DLAEYFCQQYSSYPLTFGAGTKLELKR |
| | GPNWYFDVWGPGTTVTVSSASTKGP | TVAAPSVFIFPPSDEQLKSGTASVVCLL |
| | SVFPLAPCSRSTSESTAALGCLVKDY | NNFYPREAKVQWKVDNALQSGNSQES |
| | FPEPVTVSWNSGALTSGVHTFPAVLQ | VTEQDSKDSTYSLSSTLTLSKADYEKH |
| | SSGLYSLSSVVTVPSSSLGTKTYTCN | KVYACEVTHQGLSSPVTKSFNRGEC |
| | VDHKPSNTKVDKRVESKYGPPCPPC | (SEQ ID NO: 208) |
| | PAPEFLGGPSVFLFPPKPKDTLMISRT | |
| | PEVTCVVVDVSQEDPEVQFNWYVD | |
| | GVEVHNAKTKPREEQFNSTYRVVSV | |
| | LTVLHQDWLNGKEYKCKVSNKGLP | |
| | SSIEKTISKAKGQPREPQVYTLPPSQE | |
| | EMTKNQVSLTCLVKGFYPSDIAVEW | |
| | ESNGQPENNYKTTPPVLDSDGSFFLY | |
| | SRLTVDKSRWQEGNVFSCSVMHEAL | |
| | HNHYTQKSLSLSLGK (SEQ ID NO: | |
| | 207) | |
| LK-RB2- | QIQLVQSGPELKKPGETVKISCKASG | DIKMTQSPSSMYASLGERVTITCKASQ |
| 11 | YSFITYGMSWVKQAPGKGLKWMG | DINNYLSWFQQKPGKSPKTLIFRANRL |
| | WINTYSGEPTYGDDFMGRFAFSLETS | VDGVPSRFSGSGSGQDYSLTISSLEDED |
| | ASTAYLQINNLKNEDTASYFCARYH | MGIYYCLQYDEFPWMFGGGTKLEIER |
| | YDDGMDYWGQGTSVTVSSASTKGP | TVAAPSVFIFPPSDEQLKSGTASVVCLL |
| | SVFPLAPCSRSTSESTAALGCLVKDY | NNFYPREAKVQWKVDNALQSGNSQES |
| | FPEPVTVSWNSGALTSGVHTFPAVLQ | VTEQDSKDSTYSLSSTLTLSKADYEKH |
| | SSGLYSLSSVVTVPSSSLGTKTYTCN | KVYACEVTHQGLSSPVTKSFNRGEC |
| | VDHKPSNTKVDKRVESKYGPPCPPC | (SEQ ID NO: 210) |
| | PAPEFLGGPSVFLFPPKPKDTLMISRT | |
| | PEVTCVVVDVSQEDPEVQFNWYVD | |
| | GVEVHNAKTKPREEQFNSTYRVVSV | |
| | LTVLHQDWLNGKEYKCKVSNKGLP | |
| | SSIEKTISKAKGQPREPQVYTLPPSQE | |
| | EMTKNQVSLTCLVKGFYPSDIAVEW | |
| | ESNGQPENNYKTTPPVLDSDGSFFLY | |
| | SRLTVDKSRWQEGNVFSCSVMHEAL | |
| | HNHYTQKSLSLSLGK (SEQ ID NO: | |
| | 209) | |
| LK-RB2- | QIQLVQSGPELKKPGETVKISCKASG | DIVLTQSPASLAVSPGQRATISCRASES |
| 12 | YTFTTYGMSWVKQTPGKGLKWMG | VDTYGISFMHWYQQKPGQPPKLLIYR |
| | WINIYSGVPTYADDFKGRFVFSLEISA | ASNLESGIPARFSGSGSRTDFTLTINPVE |
| | STAHLQINNLKNEDTATYFCARRKD | TDDVAIYYCQQSKKDPYTFGGGTKLEI |
| | YSGHCFDYWGQGTTLTVSSASTKGP | KRTVAAPSVFIFPPSDEQLKSGTASVVC |
| | SVFPLAPCSRSTSESTAALGCLVKDY | LLNNFYPREAKVQWKVDNALQSGNS |
| | FPEPVTVSWNSGALTSGVHTFPAVLQ | QESVTEQDSKDSTYSLSSTLTLSKADY |
| | SSGLYSLSSVVTVPSSSLGTKTYTCN | EKHKVYACEVTHQGLSSPVTKSFNRG |
| | VDHKPSNTKVDKRVESKYGPPCPPC | EC (SEQ ID NO: 212) |
| | PAPEFLGGPSVFLFPPKPKDTLMISRT | |
| | PEVTCVVVDVSQEDPEVQFNWYVD | |
| | GVEVHNAKTKPREEQFNSTYRVVSV | |
| | LTVLHQDWLNGKEYKCKVSNKGLP | |
| | SSIEKTISKAKGQPREPQVYTLPPSQE | |
| | EMTKNQVSLTCLVKGFYPSDIAVEW | |
| | ESNGQPENNYKTTPPVLDSDGSFFLY | |
| | SRLTVDKSRWQEGNVFSCSVMHEAL | |
| | HNHYTQKSLSLSLGK (SEQ ID NO: | |
| | 211) | |
| LK-RB2- | QIQLVQSGPELKKPGETVKISCKASG | DSVMSQSPSSLAVSVGEKVTMSCKSSQ |
| 13 | YTFTTYGMSWVKQAPGKGLQWMG | SLLYSGNQKSYLAWYQQKPGQSPKLLI |
| | WINTYSGVPTFADDFKGRFAFSLDTS | YWASTRESGVPDRFTGSGSGTDFTLTI |
| | ASTAYLQINNLKNEDTATYFCARRIN | SSVKAEDLAVYYCQQYYSYPYTFGGG |
| | WDGYAMDYWGQGTSVTVSSASTKG | TKLEIKRTVAAPSVFIFPPSDEQLKSGT |
| | PSVFPLAPCSRSTSESTAALGCLVKD | ASVVCLLNNFYPREAKVQWKVDNAL |
| | YFPEPVTVSWNSGALTSGVHTFPAVL | QSGNSQESVTEQDSKDSTYSLSSTLTLS |
| | QSSGLYSLSSVVTVPSSSLGTKTYTC | KADYEKHKVYACEVTHQGLSSPVTKS |
| | NVDHKPSNTKVDKRVESKYGPPCPP | FNRGEC (SEQ ID NO: 214) |
| | CPAPEFLGGPSVFLFPPKPKDTLMISR | |
| | TPEVTCVVVDVSQEDPEVQFNWYVD | |
| | GVEVHNAKTKPREEQFNSTYRVVSV | |
| | LTVLHQDWLNGKEYKCKVSNKGLP | |
| | SSIEKTISKAKGQPREPQVYTLPPSQE | |
| | EMTKNQVSLTCLVKGFYPSDIAVEW | |
| | ESNGQPENNYKTTPPVLDSDGSFFLY | |
| | SRLTVDKSRWQEGNVFSCSVMHEAL | |
| | HNHYTQKSLSLSLGK (SEQ ID NO: | |
| | 213) | |
| LK-RB2- | QVQLKQSGPGLVQPSQSLSITCTVSG | DIQMTQTTSSLSASLGDRVTISCSASQG |
| 14 | FSLTSYGVHWVRQSPGKGLEWLGVI | ISNYLNWFQQKPDGTVKLLIYYTSSLH |
| | WRGGSTDYNAAFMSRLSITKDNSKS | SGVPSRFSGSGSGTDYSLTISNLEPEDIA |
| | QVFFKMNSLQADDTAIYYCAKNEGD | TYYCQQYSKLPLTFGAGTKLELKRTV |
| | STVVAWYFDVWGTGTTVTVSSASTK | AAPSVFIFPPSDEQLKSGTASVVCLLNN |
| | GPSVFPLAPCSRSTSESTAALGCLVK | FYPREAKVQWKVDNALQSGNSQESVT |
| | DYFPEPVTVSWNSGALTSGVHTFPA | EQDSKDSTYSLSSTLTLSKADYEKHKV |
| | VLQSSGLYSLSSVVTVPSSSLGTKTY | YACEVTHQGLSSPVTKSFNRGEC (SEQ |
| | TCNVDHKPSNTKVDKRVESKYGPPC | ID NO: 216) |
| | PPCPAPEFLGGPSVFLFPPKPKDTLMI | |
| | SRTPEVTCVVVDVSQEDPEVQFNWY | |
| | VDGVEVHNAKTKPREEQFNSTYRVV | |
| | SVLTVLHQDWLNGKEYKCKVSNKG | |
| | LPSSIEKTISKAKGQPREPQVYTLPPS | |
| | QEEMTKNQVSLTCLVKGFYPSDIAV | |
| | EWESNGQPENNYKTTPPVLDSDGSFF | |
| | LYSRLTVDKSRWQEGNVFSCSVMHE | |
| | ALHNHYTQKSLSLSLGK (SEQ ID | |
| | NO: 215) | |
| LK-RB2- | QIQLVQSGPELKKPGETVKISCKASG | DIVLTQSPASLAVSLGQRATFSCRASES |
| 15 | YTFTTYGMSWVKQAPGKGLKWMG | VDTYGISFMHWYQQKPGQPPKLLIYR |
| | WINVYSGVPTYADDFKGRFAFSLEIS | ASNLESGIPARFSGSGSRTDFTLTINPVE |
| | ASTAYLQINNLKDEDTATYFCARRK | TDDVATYYCQQSKKEPYTFGGGTKLEI |
| | DYSGHCFDYWGQGTTLTVSSASTKG | KRTVAAPSVFIFPPSDEQLKSGTASVVC |
| | PSVFPLAPCSRSTSESTAALGCLVKD | LLNNFYPREAKVQWKVDNALQSGNS |
| | YFPEPVTVSWNSGALTSGVHTFPAVL | QESVTEQDSKDSTYSLSSTLTLSKADY |
| | QSSGLYSLSSVVTVPSSSLGTKTYTC | EKHKVYACEVTHQGLSSPVTKSFNRG |
| | NVDHKPSNTKVDKRVESKYGPPCPP | EC (SEQ ID NO: 218) |
| | CPAPEFLGGPSVFLFPPKPKDTLMISR | |
| | TPEVTCVVVDVSQEDPEVQFNWYVD | |
| | GVEVHNAKTKPREEQFNSTYRVVSV | |
| | LTVLHQDWLNGKEYKCKVSNKGLP | |
| | SSIEKTISKAKGQPREPQVYTLPPSQE | |
| | EMTKNQVSLTCLVKGFYPSDIAVEW | |
| | ESNGQPENNYKTTPPVLDSDGSFFLY | |
| | SRLTVDKSRWQEGNVFSCSVMHEAL | |
| | HNHYTQKSLSLSLGK (SEQ ID NO: | |
| | 217) | |
| LK-RB2- | QIQLVQSGPELKKPGETVKISCKASG | DIQMTQSPASLSASVGETVTITCRASEN |
| 16 | YTFTTYGMGWVKQAPEKGLKWMG | IYSYLAWYHQKQGESPQLLVYNAKTL |
| | WINTNSGAPTYGDDFKGRFAFSLETS | AEGVPSRFSGSGSGTQFSLKINSLQPED |
| | ASTAYLQINNLKNEDTATYFCAHYG | FGSYYCQHHYNIPLTFGAGTKLELKRT |
| | RPWFAYWGQGTLVTVSAASTKGPSV | VAAPSVFIFPPSDEQLKSGTASVVCLLN |
| | FPLAPCSRSTSESTAALGCLVKDYFP | NFYPREAKVQWKVDNALQSGNSQESV |
| | EPVTVSWNSGALTSGVHTFPAVLQSS | TEQDSKDSTYSLSSTLTLSKADYEKHK |
| | GLYSLSSVVTVPSSSLGTKTYTCNVD | VYACEVTHQGLSSPVTKSFNRGEC |
| | HKPSNTKVDKRVESKYGPPCPPCPAP | (SEQ ID NO: 220) |
| | EFLGGPSVFLFPPKPKDTLMISRTPEV | |
| | TCVVVDVSQEDPEVQFNWYVDGVE | |
| | VHNAKTKPREEQFNSTYRVVSVLTV | |
| | LHQDWLNGKEYKCKVSNKGLPSSIE | |
| | KTISKAKGQPREPQVYTLPPSQEEMT | |
| | KNQVSLTCLVKGFYPSDIAVEWESN | |
| | GQPENNYKTTPPVLDSDGSFFLYSRL | |
| | TVDKSRWQEGNVFSCSVMHEALHN | |
| | HYTQKSLSLSLGK (SEQ ID NO: 219) | |
| LK-RB2- | QIQLVQSGPELKKPGETVKISCKASG | DSVMSQSPSSLAVSVGEKVTLSCKSSQ |
| 17 | YTFTNYGMSWVKQAPGKGLQWMG | SLLYSGNQKSYLAWYQQKPGQSPKLLI |
| | WINTYSGVPTFADDFRGRFAFSLDTS | YWASSRESGVPDRFTGSGSGTDFTLTIS |
| | ASTAYLQINSLKNEDTAAYFCARRIN | SVKAEDLAVYYCQQYYSFPYTFGGGT |
| | WDGYAMDYWGQGTSVTVSSASTKG | KLEIKRTVAAPSVFIFPPSDEQLKSGTA |
| | PSVFPLAPCSRSTSESTAALGCLVKD | SVVCLLNNFYPREAKVQWKVDNALQS |
| | YFPEPVTVSWNSGALTSGVHTFPAVL | GNSQESVTEQDSKDSTYSLSSTLTLSK |
| | QSSGLYSLSSVVTVPSSSLGTKTYTC | ADYEKHKVYACEVTHQGLSSPVTKSF |
| | NVDHKPSNTKVDKRVESKYGPPCPP | NRGEC (SEQ ID NO: 222) |
| | CPAPEFLGGPSVFLFPPKPKDTLMISR | |
| | TPEVTCVVVDVSQEDPEVQFNWYVD | |
| | GVEVHNAKTKPREEQFNSTYRVVSV | |
| | LTVLHQDWLNGKEYKCKVSNKGLP | |
| | SSIEKTISKAKGQPREPQVYTLPPSQE | |
| | EMTKNQVSLTCLVKGFYPSDIAVEW | |
| | ESNGQPENNYKTTPPVLDSDGSFFLY | |
| | SRLTVDKSRWQEGNVFSCSVMHEAL | |
| | HNHYTQKSLSLSLGK (SEQ ID NO: | |
| | 221) | |
| LK-RB2- | EVQLQQSGPELVKPGTSVKISCKASG | DIQMTQSPASLSVSVGETVTITCRASEN |
| 18 | YTFTDYYMNWVKQSHGKSLEWIGDI | IYSNLAWYQQKQGKSPQLLVYGATNL |
| | NPNNGGTSYNQKFKGKATLTVDKSS | ADGVPSRFSGSGSGTQYSLKINSLQSE |
| | STAYMELRSLTSEDSAVFYCARSGTT | DFGSYYCQHFWDTPFTFGSGTKLEIKR |
| | VVGTYWYFDVWGTGTTVTVSSAST | TVAAPSVFIFPPSDEQLKSGTASVVCLL |
| | KGPSVFPLAPCSRSTSESTAALGCLV | NNFYPREAKVQWKVDNALQSGNSQES |
| | KDYFPEPVTVSWNSGALTSGVHTFP | VTEQDSKDSTYSLSSTLTLSKADYEKH |
| | AVLQSSGLYSLSSVVTVPSSSLGTKT | KVYACEVTHQGLSSPVTKSFNRGEC |
| | YTCNVDHKPSNTKVDKRVESKYGPP | (SEQ ID NO: 224) |
| | CPPCPAPEFLGGPSVFLFPPKPKDTLM | |
| | ISRTPEVTCVVVDVSQEDPEVQFNW | |
| | YVDGVEVHNAKTKPREEQFNSTYRV | |
| | VSVLTVLHQDWLNGKEYKCKVSNK | |
| | GLPSSIEKTISKAKGQPREPQVYTLPP | |
| | SQEEMTKNQVSLTCLVKGFYPSDIAV | |
| | EWESNGQPENNYKTTPPVLDSDGSFF | |
| | LYSRLTVDKSRWQEGNVFSCSVMHE | |
| | ALHNHYTQKSLSLSLGK (SEQ ID | |
| | NO: 223) | |
| LK-RB2- | QVQLQQPGAELVKPGASVKLSCKAS | QIVLSQSPAILSASPGEKVTMTCRATSR |
| 19 | GYTFTSYWMHWVKQRPGQGLEWIG | VSYIHWFQQKPGSSPKPWIYVTSNLAS |
| | MIHPNSGGTNYNEKFKSKATLTVDK | GVPARFSGSGSGTSYSLTISKLEAEDVA |
| | SSSTAYMQFSSLTSEDSAVYYCARCT | TYYCQQWSTNPLTFGAGTKLELKRTV |
| | VVALYYALDYWGQGTSVTVSSAST | AAPSVFIFPPSDEQLKSGTASVVCLLNN |
| | KGPSVFPLAPCSRSTSESTAALGCLV | FYPREAKVQWKVDNALQSGNSQESVT |
| | KDYFPEPVTVSWNSGALTSGVHTFP | EQDSKDSTYSLSSTLTLSKADYEKHKV |
| | AVLQSSGLYSLSSVVTVPSSSLGTKT | YACEVTHQGLSSPVTKSFNRGEC (SEQ |
| | YTCNVDHKPSNTKVDKRVESKYGPP | ID NO: 226) |
| | CPPCPAPEFLGGPSVFLFPPKPKDTLM | |
| | ISRTPEVTCVVVDVSQEDPEVQFNW | |
| | YVDGVEVHNAKTKPREEQFNSTYRV | |
| | VSVLTVLHQDWLNGKEYKCKVSNK | |
| | GLPSSIEKTISKAKGQPREPQVYTLPP | |
| | SQEEMTKNQVSLTCLVKGFYPSDIAV | |
| | EWESNGQPENNYKTTPPVLDSDGSFF | |
| | LYSRLTVDKSRWQEGNVFSCSVMHE | |
| | ALHNHYTQKSLSLSLGK (SEQ ID | |
| | NO: 225) | |
| LK-RB2- | QVQLKQPGAERVKPGASVKMSCKA | NIEMTQSPKSMSMAVGERVTLSCKAS |
| 20 | SGYTFTRYWITWVKQRPGQGLEWIG | ENVGTYVSWYQQKPEQSPKLLIYGAS |
| | DIYPGSATTNYNEKFKSKATLTEDTS | NRNTGVPDRFTGSGSATDFTLTISSVQ |
| | SSTAYMQLSSLTSEDSAVYYCARSG | AEDLADYHCGQSYSYPFTFGSGTKLEI |
| | DYSNSNYAMDYWGQGTSVTVSSAS | KRTVAAPSVFIFPPSDEQLKSGTASVVC |
| | TKGPSVFPLAPCSRSTSESTAALGCL | LLNNFYPREAKVQWKVDNALQSGNS |
| | VKDYFPEPVTVSWNSGALTSGVHTF | QESVTEQDSKDSTYSLSSTLTLSKADY |
| | PAVLQSSGLYSLSSVVTVPSSSLGTK | EKHKVYACEVTHQGLSSPVTKSFNRG |
| | TYTCNVDHKPSNTKVDKRVESKYGP | EC (SEQ ID NO: 228) |
| | PCPPCPAPEFLGGPSVFLFPPKPKDTL | |
| | MISRTPEVTCVVVDVSQEDPEVQFN | |
| | WYVDGVEVHNAKTKPREEQFNSTY | |
| | RVVSVLTVLHQDWLNGKEYKCKVS | |
| | NKGLPSSIEKTISKAKGQPREPQVYTL | |
| | PPSQEEMTKNQVSLTCLVKGFYPSDI | |
| | AVEWESNGQPENNYKTTPPVLDSDG | |
| | SFFLYSRLTVDKSRWQEGNVFSCSV | |
| | MHEALHNHYTQKSLSLSLGK (SEQ | |
| | ID NO: 227) | |
| LK-RB2- | EVQLVESGGDLVKPGGSLKLSCAAS | DIQMNQSPSSLSASLGDTITITCHASQNI |
| 21 | GFTFSSYGMSWVRQTPDKRLEWVAT | NVWLSWYQQKPGNIPKLLIYKASNLH |
| | ISSGGSYTYYPDSLKGRFTISRDNAKS | TGVPSRFSGSGSGTGFTLTISSLQPEDIA |
| | TLYLQMSSLKSEDTAKYYCARPYYD | TYYCQQGQSYPLTFGGGTKLEIKRTVA |
| | YIFYAIDYWGQGTSVTVSSASTKGPS | APSVFIFPPSDEQLKSGTASVVCLLNNF |
| | VFPLAPCSRSTSESTAALGCLVKDYF | YPREAKVQWKVDNALQSGNSQESVTE |
| | PEPVTVSWNSGALTSGVHTFPAVLQS | QDSKDSTYSLSSTLTLSKADYEKHKVY |
| | SGLYSLSSVVTVPSSSLGTKTYTCNV | ACEVTHQGLSSPVTKSFNRGEC (SEQ |
| | DHKPSNTKVDKRVESKYGPPCPPCP | ID NO: 230) |
| | APEFLGGPSVFLFPPKPKDTLMISRTP | |
| | EVTCVVVDVSQEDPEVQFNWYVDG | |
| | VEVHNAKTKPREEQFNSTYRVVSVL | |
| | TVLHQDWLNGKEYKCKVSNKGLPSS | |
| | IEKTISKAKGQPREPQVYTLPPSQEE | |
| | MTKNQVSLTCLVKGFYPSDIAVEWE | |
| | SNGQPENNYKTTPPVLDSDGSFFLYS | |
| | RLTVDKSRWQEGNVFSCSVMHEAL | |
| | HNHYTQKSLSLSLGK (SEQ ID NO: | |
| | 229) | |
| LK-RB2- | QIQLVQSGPELKKTGETVKISCKASG | NIVMTQTPKFLLVSAGDRVTITCRASQ |
| 22 | YTFTTYGMSWVKQAPGKGLKWMG | SVTNDVAWYQQRPGQSLKLLIYYASN |
| | WINIYSGVPTYADDFKGRFAFSLETS | RYTGVPDRFTGSGYGTDFTFTISTVQA |
| | ASTAYLQINNLKNEDTATYFCARRA | EDLAVYFCQQGYSSPFTFGSGTKLEIK |
| | DYDGYYFDYWGQGTTLTVSSASTK | RTVAAPSVFIFPPSDEQLKSGTASVVCL |
| | GPSVFPLAPCSRSTSESTAALGCLVK | LNNFYPREAKVQWKVDNALQSGNSQ |
| | DYFPEPVTVSWNSGALTSGVHTFPA | ESVTEQDSKDSTYSLSSTLTLSKADYE |
| | VLQSSGLYSLSSVVTVPSSSLGTKTY | KHKVYACEVTHQGLSSPVTKSFNRGE |
| | TCNVDHKPSNTKVDKRVESKYGPPC | C (SEQ ID NO: 232) |
| | PPCPAPEFLGGPSVFLFPPKPKDTLMI | |
| | SRTPEVTCVVVDVSQEDPEVQFNWY | |
| | VDGVEVHNAKTKPREEQFNSTYRVV | |
| | SVLTVLHQDWLNGKEYKCKVSNKG | |
| | LPSSIEKTISKAKGQPREPQVYTLPPS | |
| | QEEMTKNQVSLTCLVKGFYPSDIAV | |
| | EWESNGQPENNYKTTPPVLDSDGSFF | |
| | LYSRLTVDKSRWQEGNVFSCSVMHE | |
| | ALHNHYTQKSLSLSLGK (SEQ ID | |
| | NO: 231) | |
| LK-RB2- | QIQLVQSGPELKKPGETVKISCKASG | NIVLIQSAATLSVTPGDSVSLSCRASQS |
| 23 | YTFTTYGMSWVKQAPGKGLKWMG | LSNGLHWYQQKSHESPRLLIKYGSQSI |
| | WINIYSGVPTYADDFKGRFAFSLETS | SGIPSRFSGSGSGTDFTLSINSVETEDFG |
| | ASTAYLQINNLKNEDTATYFCARRG | MYFCQQSDSWPHTFGSGTKLEIKRTV |
| | DYDGYYFDYWGQGTTLTVSSASTK | AAPSVFIFPPSDEQLKSGTASVVCLLNN |
| | GPSVFPLAPCSRSTSESTAALGCLVK | FYPREAKVQWKVDNALQSGNSQESVT |
| | DYFPEPVTVSWNSGALTSGVHTFPA | EQDSKDSTYSLSSTLTLSKADYEKHKV |
| | VLQSSGLYSLSSVVTVPSSSLGTKTY | YACEVTHQGLSSPVTKSFNRGEC (SEQ |
| | TCNVDHKPSNTKVDKRVESKYGPPC | ID NO: 234) |
| | PPCPAPEFLGGPSVFLFPPKPKDTLMI | |
| | SRTPEVTCVVVDVSQEDPEVQFNWY | |
| | VDGVEVHNAKTKPREEQFNSTYRVV | |
| | SVLTVLHQDWLNGKEYKCKVSNKG | |
| | LPSSIEKTISKAKGQPREPQVYTLPPS | |
| | QEEMTKNQVSLTCLVKGFYPSDIAV | |
| | EWESNGQPENNYKTTPPVLDSDGSFF | |
| | LYSRLTVDKSRWQEGNVFSCSVMHE | |
| | ALHNHYTQKSLSLSLGK (SEQ ID | |
| | NO: 233) | |
| LK-RB2- | QIQLVQSGPEVKKPGETVKISCKASG | DIVMTQSPATLSVTPGDRVSLSCRASQ |
| 24 | YTFTTYGMSWVKQAPGKGLKWMG | SISDYLHWYQQKSHESPRLLIKYASQSI |
| | WLNIYSGVPAYADDFKGRFAFSLESS | SGIPSRFSGSGSGSDFTLSINSVEPEDVG |
| | ASTAYLQINNLKNEDTATYFCARRE | VYYCQNGHSFPYTFGGGTKLEIKRTVA |
| | NYDGYGMDYWGQGTSVTVSSASTK | APSVFIFPPSDEQLKSGTASVVCLLNNF |
| | GPSVFPLAPCSRSTSESTAALGCLVK | YPREAKVQWKVDNALQSGNSQESVTE |
| | DYFPEPVTVSWNSGALTSGVHTFPA | QDSKDSTYSLSSTLTLSKADYEKHKVY |
| | VLQSSGLYSLSSVVTVPSSSLGTKTY | ACEVTHQGLSSPVTKSFNRGEC (SEQ |
| | TCNVDHKPSNTKVDKRVESKYGPPC | ID NO: 236) |
| | PPCPAPEFLGGPSVFLFPPKPKDTLMI | |
| | SRTPEVTCVVVDVSQEDPEVQFNWY | |
| | VDGVEVHNAKTKPREEQFNSTYRVV | |
| | SVLTVLHQDWLNGKEYKCKVSNKG | |
| | LPSSIEKTISKAKGQPREPQVYTLPPS | |
| | QEEMTKNQVSLTCLVKGFYPSDIAV | |
| | EWESNGQPENNYKTTPPVLDSDGSFF | |
| | LYSRLTVDKSRWQEGNVFSCSVMHE | |
| | ALHNHYTQKSLSLSLGK (SEQ ID | |
| | NO: 235) | |
| LK-RB2- | QVQLKESGPGLVAPSQSLSITCTVSG | DIQMIQSPSSMFASLGDRVSLSCRASQ |
| 25 | FSLTSYGVHWVRQPPGKGLEWLGVI | GIRGNLDWYQQKPGGTIKLLIYSTSNL |
| | WAGGSTKYNSALMSRLSINKDNSKS | NSAVPSRFSGSGSGSDYSLTISNLESED |
| | QVFLKMNSLQTDDTAMYYCARDQM | FADYYCLQRNASPYTFGGGTKLEIKRT |
| | VRSAMDYWGQGTSVTVSSASTKGPS | VAAPSVFIFPPSDEQLKSGTASVVCLLN |
| | VFPLAPCSRSTSESTAALGCLVKDYF | NFYPREAKVQWKVDNALQSGNSQESV |
| | PEPVTVSWNSGALTSGVHTFPAVLQS | TEQDSKDSTYSLSSTLTLSKADYEKHK |
| | SGLYSLSSVVTVPSSSLGTKTYTCNV | VYACEVTHQGLSSPVTKSFNRGEC |
| | DHKPSNTKVDKRVESKYGPPCPPCP | (SEQ ID NO: 238) |
| | APEFLGGPSVFLFPPKPKDTLMISRTP | |
| | EVTCVVVDVSQEDPEVQFNWYVDG | |
| | VEVHNAKTKPREEQFNSTYRVVSVL | |
| | TVLHQDWLNGKEYKCKVSNKGLPSS | |
| | IEKTISKAKGQPREPQVYTLPPSQEE | |
| | MTKNQVSLTCLVKGFYPSDIAVEWE | |
| | SNGQPENNYKTTPPVLDSDGSFFLYS | |
| | RLTVDKSRWQEGNVFSCSVMHEAL | |
| | HNHYTQKSLSLSLGK (SEQ ID NO: | |
| | 237) | |
| LK-RB2- | QVQLKESGPGLVAPSQSLSITCTVSG | DIQMIQSPSSMFASLGDRVSLSCRASQ |
| 26 | FSLTSYGVHWVRQPPGKGLEWLGVI | GIRGNLDWYQQKPGGTIKLLIYSTSNL |
| | WAGGSTDYNAAFISRLNISKDISESQ | NSAVPSRFSGSGSGSDYSLTISNLESED |
| | VFFRMNSLQTDDTAMYYCARDQMV | FADYYCLQRNASPYTFGGGTKLEIKRT |
| | RSAMDYWGQGTSVTVSSASTKGPSV | VAAPSVFIFPPSDEQLKSGTASVVCLLN |
| | FPLAPCSRSTSESTAALGCLVKDYFP | NFYPREAKVQWKVDNALQSGNSQESV |
| | EPVTVSWNSGALTSGVHTFPAVLQSS | TEQDSKDSTYSLSSTLTLSKADYEKHK |
| | GLYSLSSVVTVPSSSLGTKTYTCNVD | VYACEVTHQGLSSPVTKSFNRGEC |
| | HKPSNTKVDKRVESKYGPPCPPCPAP | (SEQ ID NO: 240) |
| | EFLGGPSVFLFPPKPKDTLMISRTPEV | |
| | TCVVVDVSQEDPEVQFNWYVDGVE | |
| | VHNAKTKPREEQFNSTYRVVSVLTV | |
| | LHQDWLNGKEYKCKVSNKGLPSSIE | |
| | KTISKAKGQPREPQVYTLPPSQEEMT | |
| | KNQVSLTCLVKGFYPSDIAVEWESN | |
| | GQPENNYKTTPPVLDSDGSFFLYSRL | |
| | TVDKSRWQEGNVFSCSVMHEALHN | |
| | HYTQKSLSLSLGK (SEQ ID NO: 239) | |
| LK-RB2- | DVKLVESGGGLVKPGGSLKLSCAAS | DILMTQSPSSMSVSLGDTVSITCHASQ |
| 27 | GFTFSSYSMSWVRQTPEKRLEWVAT | DISSNIGWLQQKPGNSFKGLIYHGTNL |
| | ISTGGSYTYYPDSVKGRFTISRDNAK | EDGVPSRFSGSGSGADYSLTISSLESED |
| | NTLYLQMSSLKSEDTAMFYCTRGSM | FADYYCVQYAQFPYTFGGGTKLEIKRT |
| | DYWGQGTSVTVSSASTKGPSVFPLA | VAAPSVFIFPPSDEQLKSGTASVVCLLN |
| | PCSRSTSESTAALGCLVKDYFPEPVT | NFYPREAKVQWKVDNALQSGNSQESV |
| | VSWNSGALTSGVHTFPAVLQSSGLY | TEQDSKDSTYSLSSTLTLSKADYEKHK |
| | SLSSVVTVPSSSLGTKTYTCNVDHKP | VYACEVTHQGLSSPVTKSFNRGEC |
| | SNTKVDKRVESKYGPPCPPCPAPEFL | (SEQ ID NO: 242) |
| | GGPSVFLFPPKPKDTLMISRTPEVTCV | |
| | VVDVSQEDPEVQFNWYVDGVEVHN | |
| | AKTKPREEQFNSTYRVVSVLTVLHQ | |
| | DWLNGKEYKCKVSNKGLPSSIEKTIS | |
| | KAKGQPREPQVYTLPPSQEEMTKNQ | |
| | VSLTCLVKGFYPSDIAVEWESNGQPE | |
| | NNYKTTPPVLDSDGSFFLYSRLTVDK | |
| | SRWQEGNVFSCSVMHEALHNHYTQ | |
| | KSLSLSLGK (SEQ ID NO: 241) | |
| LK-RB2- | DVKLVESGGGLVKPGGSLKLSCAAS | DILMTQSPSSMSVSLGDTVSIACHASQ |
| 28 | GFTFSSYSMSWVRQTPEKRLEWVAT | DISSNIGWLQQKPGNSFKGLIYHGTNL |
| | ISTGGSYTYYPDSMKGRFTISRDNAK | EDGVPSRFSGSGSGADYSLTISSLESED |
| | NTLYLQMSSLKSEDTAMFYCTRGSM | FADYYCVQYAQFPYTFGGGTKLEIKRT |
| | DYWGQGTSVTVSSASTKGPSVFPLA | VAAPSVFIFPPSDEQLKSGTASVVCLLN |
| | PCSRSTSESTAALGCLVKDYFPEPVT | NFYPREAKVQWKVDNALQSGNSQESV |
| | VSWNSGALTSGVHTFPAVLQSSGLY | TEQDSKDSTYSLSSTLTLSKADYEKHK |
| | SLSSVVTVPSSSLGTKTYTCNVDHKP | VYACEVTHQGLSSPVTKSFNRGEC |
| | SNTKVDKRVESKYGPPCPPCPAPEFL | (SEQ ID NO: 244) |
| | GGPSVFLFPPKPKDTLMISRTPEVTCV | |
| | VVDVSQEDPEVQFNWYVDGVEVHN | |
| | AKTKPREEQFNSTYRVVSVLTVLHQ | |
| | DWLNGKEYKCKVSNKGLPSSIEKTIS | |
| | KAKGQPREPQVYTLPPSQEEMTKNQ | |
| | VSLTCLVKGFYPSDIAVEWESNGQPE | |
| | NNYKTTPPVLDSDGSFFLYSRLTVDK | |
| | SRWQEGNVFSCSVMHEALHNHYTQ | |
| | KSLSLSLGK (SEQ ID NO: 243) | |
| LK-RB2- | QVQLQQSGAELARPGASVKLSCKAS | DIQMTQSPASLSASVGETVTITCRASG |
| 29 | GYTFTSYWMQWVKQRPGQGLEWIG | NIHNYLAWYQQKQGKSPQLLVYNAK |
| | AIYPGDGDTRYTQKFKGKATLTADK | TLADGVPSRFSGSGSGTQYSLKINSLQP |
| | SSSTAYMQLSSLASEDSAVYYCARG | EDFGSYYCQHFWSTPPTFGGGTKLEIK |
| | YYGSSYADYFDYWGQGTTLTVSSAS | RTVAAPSVFIFPPSDEQLKSGTASVVCL |
| | TKGPSVFPLAPCSRSTSESTAALGCL | LNNFYPREAKVQWKVDNALQSGNSQ |
| | VKDYFPEPVTVSWNSGALTSGVHTF | ESVTEQDSKDSTYSLSSTLTLSKADYE |
| | PAVLQSSGLYSLSSVVTVPSSSLGTK | KHKVYACEVTHQGLSSPVTKSFNRGE |
| | TYTCNVDHKPSNTKVDKRVESKYGP | C (SEQ ID NO: 246) |
| | PCPPCPAPEFLGGPSVFLFPPKPKDTL | |
| | MISRTPEVTCVVVDVSQEDPEVQFN | |
| | WYVDGVEVHNAKTKPREEQFNSTY | |
| | RVVSVLTVLHQDWLNGKEYKCKVS | |
| | NKGLPSSIEKTISKAKGQPREPQVYTL | |
| | PPSQEEMTKNQVSLTCLVKGFYPSDI | |
| | AVEWESNGQPENNYKTTPPVLDSDG | |
| | SFFLYSRLTVDKSRWQEGNVFSCSV | |
| | MHEALHNHYTQKSLSLSLGK (SEQ | |
| | ID NO: 245) | |
| LK-RB2- | EVQLVESGGGLVQPGGSLKLSCAAS | DIQMTQSPASLSVSVGETVTITCRASEN |
| 30 | GFTFNTDGMSWVRQTPDKRLELVAII | INNNLAWYHQKQGKSPQLLVYAATNL |
| | NSNGGSTYYPDSVKGRFTISRDNAK | ADGVPSRFSGSGSGTQYSLKINSLQSE |
| | NTLYLQMSSLKSEDTAMYYCAREGS | DFGSYYCQHFWGTPFTFGGGTKLEIKR |
| | YGSWFAYWGQGTLVTVSAASTKGP | TVAAPSVFIFPPSDEQLKSGTASVVCLL |
| | SVFPLAPCSRSTSESTAALGCLVKDY | NNFYPREAKVQWKVDNALQSGNSQES |
| | FPEPVTVSWNSGALTSGVHTFPAVLQ | VTEQDSKDSTYSLSSTLTLSKADYEKH |
| | SSGLYSLSSVVTVPSSSLGTKTYTCN | KVYACEVTHQGLSSPVTKSFNRGEC |
| | VDHKPSNTKVDKRVESKYGPPCPPC | (SEQ ID NO: 248) |
| | PAPEFLGGPSVFLFPPKPKDTLMISRT | |
| | PEVTCVVVDVSQEDPEVQFNWYVD | |
| | GVEVHNAKTKPREEQFNSTYRVVSV | |
| | LTVLHQDWLNGKEYKCKVSNKGLP | |
| | SSIEKTISKAKGQPREPQVYTLPPSQE | |
| | EMTKNQVSLTCLVKGFYPSDIAVEW | |
| | ESNGQPENNYKTTPPVLDSDGSFFLY | |
| | SRLTVDKSRWQEGNVFSCSVMHEAL | |
| | HNHYTQKSLSLSLGK (SEQ ID NO: | |
| | 247) | |
| LK-RB2- | QVQLQQPGAELVKPGASMKLSCKAS | SIVMTQTPEFLLVSAGDRVTITCKASQS |
| 31 | GYTFTTYYMYWVKQRPGLGLEWIG | VNNDVAWFQQKPGQSPKLLIYYASNR |
| | GINPSNGGTTFNEKFKNKATLTVDKS | YTGVPDRFTGSGYGTDFTFTISTVQAE |
| | SSTAYMQLRSLTSEDSAVYYCTRWR | DLAIYFCQQDYSSPPTFGGATKLEIKRT |
| | FFDVWGAGTTVTVSSASTKGPSVFPL | VAAPSVFIFPPSDEQLKSGTASVVCLLN |
| | APCSRSTSESTAALGCLVKDYFPEPV | NFYPREAKVQWKVDNALQSGNSQESV |
| | TVSWNSGALTSGVHTFPAVLQSSGL | TEQDSKDSTYSLSSTLTLSKADYEKHK |
| | YSLSSVVTVPSSSLGTKTYTCNVDHK | VYACEVTHQGLSSPVTKSFNRGEC |
| | PSNTKVDKRVESKYGPPCPPCPAPEF | (SEQ ID NO: 250) |
| | LGGPSVFLFPPKPKDTLMISRTPEVTC | |
| | VVVDVSQEDPEVQFNWYVDGVEVH | |
| | NAKTKPREEQFNSTYRVVSVLTVLH | |
| | QDWLNGKEYKCKVSNKGLPSSIEKTI | |
| | SKAKGQPREPQVYTLPPSQEEMTKN | |
| | QVSLTCLVKGFYPSDIAVEWESNGQP | |
| | ENNYKTTPPVLDSDGSFFLYSRLTVD | |
| | KSRWQEGNVFSCSVMHEALHNHYT | |
| | QKSLSLSLGK (SEQ ID NO: 249) | |
| LK-RB2- | DVQLQESGPGLVKPSQSLSLTCSVTG | DIVLTQSPVTLSVTPGDSVSLSCRASQN |
| 32 | YSITSGYYWNWIRQFPGNKLEWMG | INKNLHWYQQRSHESPRLLIKYVSQSIS |
| | YISFDGSNHYNPSLRNRISITRDTFKN | GIPSRFSGSGSGTDFTLSINSVETEDFG |
| | QFFLKLNSVTTEDTATFYCTRRDYN | MYFCQQSNSWPLTFGAGTKLELRRTV |
| | WFAYWGQGTLVTVSAASTKGPSVFP | AAPSVFIFPPSDEQLKSGTASVVCLLNN |
| | LAPCSRSTSESTAALGCLVKDYFPEP | FYPREAKVQWKVDNALQSGNSQESVT |
| | VTVSWNSGALTSGVHTFPAVLQSSG | EQDSKDSTYSLSSTLTLSKADYEKHKV |
| | LYSLSSVVTVPSSSLGTKTYTCNVDH | YACEVTHQGLSSPVTKSFNRGEC (SEQ |
| | KPSNTKVDKRVESKYGPPCPPCPAPE | ID NO: 252) |
| | FLGGPSVFLFPPKPKDTLMISRTPEVT | |
| | CVVVDVSQEDPEVQFNWYVDGVEV | |
| | HNAKTKPREEQFNSTYRVVSVLTVL | |
| | HQDWLNGKEYKCKVSNKGLPSSIEK | |
| | TISKAKGQPREPQVYTLPPSQEEMTK | |
| | NQVSLTCLVKGFYPSDIAVEWESNG | |
| | QPENNYKTTPPVLDSDGSFFLYSRLT | |
| | VDKSRWQEGNVFSCSVMHEALHNH | |
| | YTQKSLSLSLGK (SEQ ID NO: 251) | |
| LK-RB2- | EVQLQQSGVELVRPGALVKLSCKAS | DVVMTQSHKFMSTSVGDRVTITCKAS |
| 33 | GFNIKDYYMHWVKQRPEQGLEWIG | QDVSNAVAWYQQKPGQSPKLLIYSAS |
| | WIDPEKGDTIYGPKFQGKASITADTS | YRYTGVPDRFTGSGSGTDFTFTISSMQ |
| | SNTAYLQLSSLTSEDTAVYYCSRGPN | AEDLAVYYCQQHYSLPWTFGGGTKLE |
| | YGSSYAFAYWGQGTLVTVSAASTKG | IKRTVAAPSVFIFPPSDEQLKSGTASVV |
| | PSVFPLAPCSRSTSESTAALGCLVKD | CLLNNFYPREAKVQWKVDNALQSGNS |
| | YFPEPVTVSWNSGALTSGVHTFPAVL | QESVTEQDSKDSTYSLSSTLTLSKADY |
| | QSSGLYSLSSVVTVPSSSLGTKTYTC | EKHKVYACEVTHQGLSSPVTKSFNRG |
| | NVDHKPSNTKVDKRVESKYGPPCPP | EC (SEQ ID NO: 254) |
| | CPAPEFLGGPSVFLFPPKPKDTLMISR | |
| | TPEVTCVVVDVSQEDPEVQFNWYVD | |
| | GVEVHNAKTKPREEQFNSTYRVVSV | |
| | LTVLHQDWLNGKEYKCKVSNKGLP | |
| | SSIEKTISKAKGQPREPQVYTLPPSQE | |
| | EMTKNQVSLTCLVKGFYPSDIAVEW | |
| | ESNGQPENNYKTTPPVLDSDGSFFLY | |
| | SRLTVDKSRWQEGNVFSCSVMHEAL | |
| | HNHYTQKSLSLSLGK (SEQ ID NO: | |
| | 253) | |
| LK-RB2- | DVKVVESGGGLVKPGGSLKLSCAAS | DILMTQSPSSMSVSLGDTVSITCHASQ |
| 34 | GFTFSSYSMSWVRQTPEKRLEWVAT | DISSNIGWLQQKPGNSFKGLIYHGINLE |
| | ISTGSSYTYYSDSVKGRFTISRDNAK | DGVPSRFSGSGSGADYSLTISSLESEDF |
| | NTLYLQMSSLKSEDTAMFYCTRGSM | ADYYCVQYAQFPYTFGGGTKLEIKRT |
| | DYWGQGTSVTVSSASTKGPSVFPLA | VAAPSVFIFPPSDEQLKSGTASVVCLLN |
| | PCSRSTSESTAALGCLVKDYFPEPVT | NFYPREAKVQWKVDNALQSGNSQESV |
| | VSWNSGALTSGVHTFPAVLQSSGLY | TEQDSKDSTYSLSSTLTLSKADYEKHK |
| | SLSSVVTVPSSSLGTKTYTCNVDHKP | VYACEVTHQGLSSPVTKSFNRGEC |
| | SNTKVDKRVESKYGPPCPPCPAPEFL | (SEQ ID NO: 256) |
| | GGPSVFLFPPKPKDTLMISRTPEVTCV | |
| | VVDVSQEDPEVQFNWYVDGVEVHN | |
| | AKTKPREEQFNSTYRVVSVLTVLHQ | |
| | DWLNGKEYKCKVSNKGLPSSIEKTIS | |
| | KAKGQPREPQVYTLPPSQEEMTKNQ | |
| | VSLTCLVKGFYPSDIAVEWESNGQPE | |
| | NNYKTTPPVLDSDGSFFLYSRLTVDK | |
| | SRWQEGNVFSCSVMHEALHNHYTQ | |
| | KSLSLSLGK (SEQ ID NO: 255) | |
| LK-RB2- | EVQLQQSGPELVKPGASVKISCKASG | DIVMTQSPATLSVTPGDRVSLSCRASQ |
| 35 | YTFTDYNMHWVKQSHGKSLEWIGSI | SISDYLHWYQQKSHESPRLLIKYASQSI |
| | YPYNGGSDYNQKFKSKATLTVDNSS | SGIPSRFSGSGSGSDFTLSINSVEPEDVG |
| | STAYMELRSLTSEDSAVYYCASELTG | VYYCQTGHSFPYTFGGGTKLEIKRTVA |
| | LYAMDYWGQGTSVTVSSASTKGPSV | APSVFIFPPSDEQLKSGTASVVCLLNNF |
| | FPLAPCSRSTSESTAALGCLVKDYFP | YPREAKVQWKVDNALQSGNSQESVTE |
| | EPVTVSWNSGALTSGVHTFPAVLQSS | QDSKDSTYSLSSTLTLSKADYEKHKVY |
| | GLYSLSSVVTVPSSSLGTKTYTCNVD | ACEVTHQGLSSPVTKSFNRGEC (SEQ |
| | HKPSNTKVDKRVESKYGPPCPPCPAP | ID NO: 258) |
| | EFLGGPSVFLFPPKPKDTLMISRTPEV | |
| | TCVVVDVSQEDPEVQFNWYVDGVE | |
| | VHNAKTKPREEQFNSTYRVVSVLTV | |
| | LHQDWLNGKEYKCKVSNKGLPSSIE | |
| | KTISKAKGQPREPQVYTLPPSQEEMT | |
| | KNQVSLTCLVKGFYPSDIAVEWESN | |
| | GQPENNYKTTPPVLDSDGSFFLYSRL | |
| | TVDKSRWQEGNVFSCSVMHEALHN | |
| | HYTQKSLSLSLGK (SEQ ID NO: 257) | |
| LK-RB2- | QVQLQQSGAELVRPGASVKMSCKAS | DIVLTQSPASLSVTPGDSVSLSCRASQS |
| 36 | GYTFTNYWIGWAKQRPGHGLEWIG | ISNNLHWYQQKSHESPRLLIKYASQSIS |
| | DIYPGGTYTGYNEKFKGKATLTADK | GIPSRFSGSGSGTDFTLSINSVETEDFG |
| | SSSTAYMQFSSLTSEDSAIYYCARRM | MYFCQQSNSWPLTFGAGTKLELKRTV |
| | DYDGYAMDYWGQGTSVTVSSASTK | AAPSVFIFPPSDEQLKSGTASVVCLLNN |
| | GPSVFPLAPCSRSTSESTAALGCLVK | FYPREAKVQWKVDNALQSGNSQESVT |
| | DYFPEPVTVSWNSGALTSGVHTFPA | EQDSKDSTYSLSSTLTLSKADYEKHKV |
| | VLQSSGLYSLSSVVTVPSSSLGTKTY | YACEVTHQGLSSPVTKSFNRGEC (SEQ |
| | TCNVDHKPSNTKVDKRVESKYGPPC | ID NO: 260) |
| | PPCPAPEFLGGPSVFLFPPKPKDTLMI | |
| | SRTPEVTCVVVDVSQEDPEVQFNWY | |
| | VDGVEVHNAKTKPREEQFNSTYRVV | |
| | SVLTVLHQDWLNGKEYKCKVSNKG | |
| | LPSSIEKTISKAKGQPREPQVYTLPPS | |
| | QEEMTKNQVSLTCLVKGFYPSDIAV | |
| | EWESNGQPENNYKTTPPVLDSDGSFF | |
| | LYSRLTVDKSRWQEGNVFSCSVMHE | |
| | ALHNHYTQKSLSLSLGK (SEQ ID | |
| | NO: 259) | |
| LK-RB2- | QIQLVQSGPEVKKPGETVKISCKASG | DIVMSQSPSSLAVSVGEKVTMSCKSSQ |
| 37 | YTFTTYGMSWVKQAPGKGLKWMG | SLLYSGNQKNYLAWYQQKPGQSPKLL |
| | WINTYSGVPTYADDFKGRFAFSLETS | IYWASARESGVPDRFTGSGSGTDFTLTI |
| | VSTAYLQINNLKNEDTSTYFCARRRD | SSVKAEDLAVYYCQQYYTYPFTFGSG |
| | YDGYAMDYWGQGTSVTVSSASTKG | TKLEIKRTVAAPSVFIFPPSDEQLKSGT |
| | PSVFPLAPCSRSTSESTAALGCLVKD | ASVVCLLNNFYPREAKVQWKVDNAL |
| | YFPEPVTVSWNSGALTSGVHTFPAVL | QSGNSQESVTEQDSKDSTYSLSSTLTLS |
| | QSSGLYSLSSVVTVPSSSLGTKTYTC | KADYEKHKVYACEVTHQGLSSPVTKS |
| | NVDHKPSNTKVDKRVESKYGPPCPP | FNRGEC (SEQ ID NO: 262) |
| | CPAPEFLGGPSVFLFPPKPKDTLMISR | |
| | TPEVTCVVVDVSQEDPEVQFNWYVD | |
| | GVEVHNAKTKPREEQFNSTYRVVSV | |
| | LTVLHQDWLNGKEYKCKVSNKGLP | |
| | SSIEKTISKAKGQPREPQVYTLPPSQE | |
| | EMTKNQVSLTCLVKGFYPSDIAVEW | |
| | ESNGQPENNYKTTPPVLDSDGSFFLY | |
| | SRLTVDKSRWQEGNVFSCSVMHEAL | |
| | HNHYTQKSLSLSLGK (SEQ ID NO: | |
| | 261) | |
| LK-RB2- | EVKLVESGGGLVQPGGSLKLSCAAS | DIVMTQSHTFMSTSVGDRVSITCKASQ |
| 38 | GFSFSDYYMYWVRQTPEKRLEWVA | DVSTAVAWYQQKPGQSPKLLIYWAST |
| | YISNGGGNTYYPDTVKGRFTISRDNA | RHTGVPDRFTGSGSGTDYTLTISSVQA |
| | KNSLYLQMSRLKSEDTGMYYCARD | EDLALYYCQQHYSTPRTFGGGTKLEIK |
| | REDWYFDVWGTGTTVTVSSASTKGP | RTVAAPSVFIFPPSDEQLKSGTASVVCL |
| | SVFPLAPCSRSTSESTAALGCLVKDY | LNNFYPREAKVQWKVDNALQSGNSQ |
| | FPEPVTVSWNSGALTSGVHTFPAVLQ | ESVTEQDSKDSTYSLSSTLTLSKADYE |
| | SSGLYSLSSVVTVPSSSLGTKTYTCN | KHKVYACEVTHQGLSSPVTKSFNRGE |
| | VDHKPSNTKVDKRVESKYGPPCPPC | C (SEQ ID NO: 264) |
| | PAPEFLGGPSVFLFPPKPKDTLMISRT | |
| | PEVTCVVVDVSQEDPEVQFNWYVD | |
| | GVEVHNAKTKPREEQFNSTYRVVSV | |
| | LTVLHQDWLNGKEYKCKVSNKGLP | |
| | SSIEKTISKAKGQPREPQVYTLPPSQE | |
| | EMTKNQVSLTCLVKGFYPSDIAVEW | |
| | ESNGQPENNYKTTPPVLDSDGSFFLY | |
| | SRLTVDKSRWQEGNVFSCSVMHEAL | |
| | HNHYTQKSLSLSLGK (SEQ ID NO: | |
| | 263) | |
| LK-RB2- | DVKLVESGGGSVQPGGSLKLSCAAS | DIVMTQSPSSLAMSVGQKVTMSCKSS |
| 39 | GFTFSDYYMYWVRQTPEKRLEWVA | QSLLHSSNQKNYLAWYQQKPGQSPKL |
| | YISNGGGNTYYPDTVKGRFTISRDNA | LVSFASTRESGVPDRFIGSGSGTDFTLTI |
| | KNTLYLQMSRLKSEDTAMYYCARS | NSVQAEDLADYFCQQHYSTPLTFGAG |
| | NWAWYFDVWGTGTTVTVSSASTKG | TKLELKRTVAAPSVFIFPPSDEQLKSGT |
| | PSVFPLAPCSRSTSESTAALGCLVKD | ASVVCLLNNFYPREAKVQWKVDNAL |
| | YFPEPVTVSWNSGALTSGVHTFPAVL | QSGNSQESVTEQDSKDSTYSLSSTLTLS |
| | QSSGLYSLSSVVTVPSSSLGTKTYTC | KADYEKHKVYACEVTHQGLSSPVTKS |
| | NVDHKPSNTKVDKRVESKYGPPCPP | FNRGEC (SEQ ID NO: 266) |
| | CPAPEFLGGPSVFLFPPKPKDTLMISR | |
| | TPEVTCVVVDVSQEDPEVQFNWYVD | |
| | GVEVHNAKTKPREEQFNSTYRVVSV | |
| | LTVLHQDWLNGKEYKCKVSNKGLP | |
| | SSIEKTISKAKGQPREPQVYTLPPSQE | |
| | EMTKNQVSLTCLVKGFYPSDIAVEW | |
| | ESNGQPENNYKTTPPVLDSDGSFFLY | |
| | SRLTVDKSRWQEGNVFSCSVMHEAL | |
| | HNHYTQKSLSLSLGK (SEQ ID NO: | |
| | 265) | |
| LK-RB2- | DVQLQESGPGLVKPSQSLSLTCSVTG | DIQMTQSPASLSVSVGETVTITCRASEN |
| 40 | YSITSGYYWNWIRQFPGNKLEWMA | IYSNLAWYQQKQGKSPQLLVYAATNL |
| | YISYDGSNNYNPSLKNRISITRDTSKN | ADGVPSRFSGSGSGTQYSLKINSLQSE |
| | QFFLKLNSVTTEDTATYYCAREEGT | DFGSYYCQHFWDTPFTFGSGTKLEIKR |
| | AQATLGYFDYWGQGTTLTVSSASTK | TVAAPSVFIFPPSDEQLKSGTASVVCLL |
| | GPSVFPLAPCSRSTSESTAALGCLVK | NNFYPREAKVQWKVDNALQSGNSQES |
| | DYFPEPVTVSWNSGALTSGVHTFPA | VTEQDSKDSTYSLSSTLTLSKADYEKH |
| | VLQSSGLYSLSSVVTVPSSSLGTKTY | KVYACEVTHQGLSSPVTKSFNRGEC |
| | TCNVDHKPSNTKVDKRVESKYGPPC | (SEQ ID NO: 268) |
| | PPCPAPEFLGGPSVFLFPPKPKDTLMI | |
| | SRTPEVTCVVVDVSQEDPEVQFNWY | |
| | VDGVEVHNAKTKPREEQFNSTYRVV | |
| | SVLTVLHQDWLNGKEYKCKVSNKG | |
| | LPSSIEKTISKAKGQPREPQVYTLPPS | |
| | QEEMTKNQVSLTCLVKGFYPSDIAV | |
| | EWESNGQPENNYKTTPPVLDSDGSFF | |
| | LYSRLTVDKSRWQEGNVFSCSVMHE | |
| | ALHNHYTQKSLSLSLGK (SEQ ID | |
| | NO: 267) | |
| LK-RB2- | EVQLQLSGPELVKPGASVKMSCKAS | DIVMTQSHKFMSTSVGDRVSITCKASQ |
| 41 | GYTFTDYNMHWVKQSHGKSLEWIG | DVSTAVAWYQQKPGQSPKLLIYSASY |
| | YINPNNGGTTYNQKFKGKATLTVNK | RYTGVPDRFTGSGSGTDFTFTISSVQAE |
| | SSSTAYMELRSLTSEDSAVYYCARAP | DLAIYYCQQHYSIPWTFGGGTKLEIKR |
| | YYGSSYLLAYWGQGTLVTVSAASTK | TVAAPSVFIFPPSDEQLKSGTASVVCLL |
| | GPSVFPLAPCSRSTSESTAALGCLVK | NNFYPREAKVQWKVDNALQSGNSQES |
| | DYFPEPVTVSWNSGALTSGVHTFPA | VTEQDSKDSTYSLSSTLTLSKADYEKH |
| | VLQSSGLYSLSSVVTVPSSSLGTKTY | KVYACEVTHQGLSSPVTKSFNRGEC |
| | TCNVDHKPSNTKVDKRVESKYGPPC | (SEQ ID NO: 270) |
| | PPCPAPEFLGGPSVFLFPPKPKDTLMI | |
| | SRTPEVTCVVVDVSQEDPEVQFNWY | |
| | VDGVEVHNAKTKPREEQFNSTYRVV | |
| | SVLTVLHQDWLNGKEYKCKVSNKG | |
| | LPSSIEKTISKAKGQPREPQVYTLPPS | |
| | QEEMTKNQVSLTCLVKGFYPSDIAV | |
| | EWESNGQPENNYKTTPPVLDSDGSFF | |
| | LYSRLTVDKSRWQEGNVFSCSVMHE | |
| | ALHNHYTQKSLSLSLGK (SEQ ID | |
| | NO: 269) | |
| LK-RB2- | EFQLQQSGPELVKPGASVKISCKASG | DVLMTQTPLSLPVSLGDQASISCRSSQS |
| 42 | YSFTDYNMNWVKQSNGKSLDWIGII | IVHSNGNTYLEWYLQKPGQPPKLLIYK |
| | NPKYGTTAYNQKLRDKATLTVDQSS | VSNRFSGVPERFSGSGSGTDFTLKINRV |
| | STAYMQLNSLTSEDSAVYYCASLNW | EAEDLGVYYCFQGSLVPPTFGGGTKLE |
| | DEGFAYWGQGTLVTVSPASTKGPSV | IKRTVAAPSVFIFPPSDEQLKSGTASVV |
| | FPLAPCSRSTSESTAALGCLVKDYFP | CLLNNFYPREAKVQWKVDNALQSGNS |
| | EPVTVSWNSGALTSGVHTFPAVLQSS | QESVTEQDSKDSTYSLSSTLTLSKADY |
| | GLYSLSSVVTVPSSSLGTKTYTCNVD | EKHKVYACEVTHQGLSSPVTKSFNRG |
| | HKPSNTKVDKRVESKYGPPCPPCPAP | EC (SEQ ID NO: 272) |
| | EFLGGPSVFLFPPKPKDTLMISRTPEV | |
| | TCVVVDVSQEDPEVQFNWYVDGVE | |
| | VHNAKTKPREEQFNSTYRVVSVLTV | |
| | LHQDWLNGKEYKCKVSNKGLPSSIE | |
| | KTISKAKGQPREPQVYTLPPSQEEMT | |
| | KNQVSLTCLVKGFYPSDIAVEWESN | |
| | GQPENNYKTTPPVLDSDGSFFLYSRL | |
| | TVDKSRWQEGNVFSCSVMHEALHN | |
| | HYTQKSLSLSLGK (SEQ ID NO: 271) | |
| LK-RB2- | EVQLVESGGDLVKPGGSLKLSCAAS | QIVLTQSPAILSASPGEKVTMTCSANSS |
| 43 | GFTFSSYGMSWVRQTPDKRLEWVAT | VSSMYKYQQKPGSSPKPWIYGTSNLAS |
| | ISGGGTYTYYPDSVKGRFTISRDNAK | GVPARFSGSGSGTSYSLTISSMEAADA |
| | NTLYLQMSSLKSEDTAMYYCASGA | ATYYCQQYHSHPLTFGAGTKLELKRT |
| | MDYWGQGTSVTVSSASTKGPSVFPL | VAAPSVFIFPPSDEQLKSGTASVVCLLN |
| | APCSRSTSESTAALGCLVKDYFPEPV | NFYPREAKVQWKVDNALQSGNSQESV |
| | TVSWNSGALTSGVHTFPAVLQSSGL | TEQDSKDSTYSLSSTLTLSKADYEKHK |
| | YSLSSVVTVPSSSLGTKTYTCNVDHK | VYACEVTHQGLSSPVTKSFNRGEC |
| | PSNTKVDKRVESKYGPPCPPCPAPEF | (SEQ ID NO: 274) |
| | LGGPSVFLFPPKPKDTLMISRTPEVTC | |
| | VVVDVSQEDPEVQFNWYVDGVEVH | |
| | NAKTKPREEQFNSTYRVVSVLTVLH | |
| | QDWLNGKEYKCKVSNKGLPSSIEKTI | |
| | SKAKGQPREPQVYTLPPSQEEMTKN | |
| | QVSLTCLVKGFYPSDIAVEWESNGQP | |
| | ENNYKTTPPVLDSDGSFFLYSRLTVD | |
| | KSRWQEGNVFSCSVMHEALHNHYT | |
| | QKSLSLSLGK (SEQ ID NO: 273) | |
| LK-RB2- | EVQLVESGGDLVKPGGSLKLSCAAS | DVVMTQTPLTLSVTIGQPASISCKSSQS |
| 44 | GFTFSSYGMSWVRQTPDKRLEWVAT | LLDIDGKTFLNWLLQRPGQSPKRLIYL |
| | ISGGGTYTYYPDSVKGRFTISRDNAK | VSKLDSGVPDRFTGSGSGTDFTLKISR |
| | NTLYLQMSSLKSEDTAMYYCASGA | VEAEDLGVYYCWQGTHFPWTFGGGT |
| | MDYWGQGTSVTVSSASTKGPSVFPL | KLEIKRTVAAPSVFIFPPSDEQLKSGTA |
| | APCSRSTSESTAALGCLVKDYFPEPV | SVVCLLNNFYPREAKVQWKVDNALQS |
| | TVSWNSGALTSGVHTFPAVLQSSGL | GNSQESVTEQDSKDSTYSLSSTLTLSK |
| | YSLSSVVTVPSSSLGTKTYTCNVDHK | ADYEKHKVYACEVTHQGLSSPVTKSF |
| | PSNTKVDKRVESKYGPPCPPCPAPEF | NRGEC (SEQ ID NO: 276) |
| | LGGPSVFLFPPKPKDTLMISRTPEVTC | |
| | VVVDVSQEDPEVQFNWYVDGVEVH | |
| | NAKTKPREEQFNSTYRVVSVLTVLH | |
| | QDWLNGKEYKCKVSNKGLPSSIEKTI | |
| | SKAKGQPREPQVYTLPPSQEEMTKN | |
| | QVSLTCLVKGFYPSDIAVEWESNGQP | |
| | ENNYKTTPPVLDSDGSFFLYSRLTVD | |
| | KSRWQEGNVFSCSVMHEALHNHYT | |
| | QKSLSLSLGK (SEQ ID NO: 275) | |
| LK-RB2- | QIQLVQSGPELKKPGETVKISCKASG | DIVMTQSPATLSVTPGDRVSLSCRASQ |
| 45 | YTFTTYGMSWVKQTPGKGLQWMG | SISDYLNWYQQKSHESPRLLIKYVSQSI |
| | WINVYSGVPTYADDFKGRFAFSLEA | SGIPSRFSGSGSGSDFTLSINSVEPEDIG |
| | SASTAYLQINNLKNEDTASYFCARRG | VYYCQNGHNFPLTFGGGTKLELKRTV |
| | DYDGHYFDYWGQGTTLTVSSASTK | AAPSVFIFPPSDEQLKSGTASVVCLLNN |
| | GPSVFPLAPCSRSTSESTAALGCLVK | FYPREAKVQWKVDNALQSGNSQESVT |
| | DYFPEPVTVSWNSGALTSGVHTFPA | EQDSKDSTYSLSSTLTLSKADYEKHKV |
| | VLQSSGLYSLSSVVTVPSSSLGTKTY | YACEVTHQGLSSPVTKSFNRGEC (SEQ |
| | TCNVDHKPSNTKVDKRVESKYGPPC | ID NO: 278) |
| | PPCPAPEFLGGPSVFLFPPKPKDTLMI | |
| | SRTPEVTCVVVDVSQEDPEVQFNWY | |
| | VDGVEVHNAKTKPREEQFNSTYRVV | |
| | SVLTVLHQDWLNGKEYKCKVSNKG | |
| | LPSSIEKTISKAKGQPREPQVYTLPPS | |
| | QEEMTKNQVSLTCLVKGFYPSDIAV | |
| | EWESNGQPENNYKTTPPVLDSDGSFF | |
| | LYSRLTVDKSRWQEGNVFSCSVMHE | |
| | ALHNHYTQKSLSLSLGK (SEQ ID | |
| | NO: 277) | |
| LK-RB2- | QVQMQQSDAELVKPGASVKISCKVS | DIVMTQSHKFMSASVGDRVSITCKASQ |
| 46 | GYTFTDQIIHWMKQRPEQGLEWIGYI | DVNTAVAWYRQDPGQSPKLLIYWAST |
| | YPRDDSIMYNEKFKGKATLTADKSS | RHTGVPDRFTGSGSGTDYTLTINNFQA |
| | STANMQLNSLTSEDSAVYFCAYDAY | EDLALYYCQQHYSPPLTFGAGTKLELK |
| | WYFDVWGTGTTVIVSSASTKGPSVF | RTVAAPSVFIFPPSDEQLKSGTASVVCL |
| | PLAPCSRSTSESTAALGCLVKDYFPE | LNNFYPREAKVQWKVDNALQSGNSQ |
| | PVTVSWNSGALTSGVHTFPAVLQSS | ESVTEQDSKDSTYSLSSTLTLSKADYE |
| | GLYSLSSVVTVPSSSLGTKTYTCNVD | KHKVYACEVTHQGLSSPVTKSFNRGE |
| | HKPSNTKVDKRVESKYGPPCPPCPAP | C (SEQ ID NO: 280) |
| | EFLGGPSVFLFPPKPKDTLMISRTPEV | |
| | TCVVVDVSQEDPEVQFNWYVDGVE | |
| | VHNAKTKPREEQFNSTYRVVSVLTV | |
| | LHQDWLNGKEYKCKVSNKGLPSSIE | |
| | KTISKAKGQPREPQVYTLPPSQEEMT | |
| | KNQVSLTCLVKGFYPSDIAVEWESN | |
| | GQPENNYKTTPPVLDSDGSFFLYSRL | |
| | TVDKSRWQEGNVFSCSVMHEALHN | |
| | HYTQKSLSLSLGK (SEQ ID NO: 279) | |
| LK-RB2- | QVQLKQSGPGLVQPSQSLSITCTVSG | DIQMTQTTSSLSASLGDRVIISCRASQD |
| 47 | FSLISYGVHWVRQSPGKGLEWLGVI | INNYLNWYQQKPDGTVKLLIYFTSRLH |
| | WSGGSTDYNAAFISRLSISKDNSKSQ | SGVPSSFSGSGSGTDYSLTINNLEQEDI |
| | VFFKMNSLQADDTAIYYCARSFHYH | ATYFCQQVNTLPYTFGGGTKLEIKRTV |
| | GSTFDYWGQGTTLTVSSASTKGPSVF | AAPSVFIFPPSDEQLKSGTASVVCLLNN |
| | PLAPCSRSTSESTAALGCLVKDYFPE | FYPREAKVQWKVDNALQSGNSQESVT |
| | PVTVSWNSGALTSGVHTFPAVLQSS | EQDSKDSTYSLSSTLTLSKADYEKHKV |
| | GLYSLSSVVTVPSSSLGTKTYTCNVD | YACEVTHQGLSSPVTKSFNRGEC (SEQ |
| | HKPSNTKVDKRVESKYGPPCPPCPAP | ID NO: 282) |
| | EFLGGPSVFLFPPKPKDTLMISRTPEV | |
| | TCVVVDVSQEDPEVQFNWYVDGVE | |
| | VHNAKTKPREEQFNSTYRVVSVLTV | |
| | LHQDWLNGKEYKCKVSNKGLPSSIE | |
| | KTISKAKGQPREPQVYTLPPSQEEMT | |
| | KNQVSLTCLVKGFYPSDIAVEWESN | |
| | GQPENNYKTTPPVLDSDGSFFLYSRL | |
| | TVDKSRWQEGNVFSCSVMHEALHN | |
| | HYTQKSLSLSLGK (SEQ ID NO: 281) | |

TABLE 5
VH and VL sequences of anti-LILRB1 (LK-RB1-1 to LK-RB1-59) and anti-
LILRB2 (LK-RB2-1 to LK-RB2-47) antibodies

| Antibody | VH | VL |
|---|---|---|
| LK-RB1-1 | QIQLVQSGPELKKPGETVKISCKAS | QLVLTQSSSASFSLGASAKLTCTLSSQHS |
| | GYSFTNFGMSWVKQAPGKALKWM | TYSIDWYQQQPLKPPKYVMELKTDGSH |
| | GWINTYSGVPTYADDFKGRFAFSL | RTGDGIPDRFSGSSSGADRYLSISNIQPE |
| | ETSANTAYLQINNLKNEDSATYFCA | DEAIYICGVGDTIKEQFVYVFGGGTKVT |
| | RRRRTFDGYLCFDYWGQGATLTVS | VL (SEQ ID NO: 284) |
| | S (SEQ ID NO: 283) | |
| LK-RB1-2 | QIQLVQSGPELKKPGETVKISCKAS | DIQMTQSPGSLSASVGETVTITCGASENI |
| | GYTFTTYGVSWVKQAPGKGLKWM | YGALNWFQRKPGKSPQLLIYGATNLAD |
| | GWINTYSGVSTYSDDFKGRFAFSLE | GMSSRFSGSGSGRQYSLQISSLRPDDVA |
| | TSASTAYLQIINLKNEDAATYFCSR | TYYCQNVLTTPWTFGGGTKVEIK (SEQ |
| | RRRTYDGYLSFDYWGQGTTLTVSS | ID NO: 286) |
| | (SEQ ID NO: 285) | |
| LK-RB1-3 | QVQLQQPGAELVKPGTSVNLSCKA | DIVMTQSQKFMPTSVGDRVNVTCKASQ |
| | SGYTFTSYWIHWVKQRPGRGLEWI | NVGINVAWYQQKLGQSPKALIYSASYR |
| | GRINPYSSGTRYNEKFKNKATLTVD | YSGVPDRFTGSGSGTDFTLTINSVQSEDL |
| | KPSSTAYMQLTSLTSEDSAVYYCA | ADYFCQQYNNYPFTFGSGTKLEIK (SEQ |
| | RFDGAPDYFDFWGQGTTLTVSS | ID NO: 288) |
| | (SEQ ID NO: 287) | |
| LK-RB1-4 | QVQLQQPGAELVKPGASVKLPCKT | DIVMTQSQKFMSTSVGDRVSVTCKASQ |
| | SGYTFTSYWMHWVKQRPGRGLEW | NVGTNVAWYKQKAGQSPKAVIYSASY |
| | IGRIDPHYSGTKYNEKFKNKATMT | RYSGVPDRFTGSGSGTDFTLSISNVQSED |
| | VDTPSSTAYMQLSSLTSEDSAVYYC | LAEYFCQQYNNNPFTFGSGTKLEIK |
| | ARFDGDPYYFDYWGPGTTLTVSS | (SEQ ID NO: 290) |
| | (SEQ ID NO: 289) | |
| LK-RB1-5 | QVQLQQPGAELVKPGASVKLSCKA | QIVLSQSPAILSASPGEKVTLTCRASSSVS |
| | SGYTFTDFWIHWVKQRPGQGLEWI | YMHWYQQKPGSSPKPWIYATSNLASGV |
| | GMIHPNSGSTNFNEKFRAKATLTV | PVRFSGSGSGTSYSLTISRMQTEDAATY |
| | DKSSSTAYMRLSSLTSEDSAVYYC | YCQQWRRSPYTFGGGTKLEIK (SEQ ID |
| | ARSGDGYSNPMDYWGQGTSVTVS | NO: 292) |
| | S (SEQ ID NO: 291) | |
| LK-RB1-6 | QIQLVQSGPELKKPGETVKISCKAS | DIVMTQSHKFMSTSVGDRVSITCTASED |
| | GYTFTTYGMSWVKQAPGKDLKW | VNTAVAWYQQKPGQSPKLLIYWASTRH |
| | MGWINTFSGVPACADDFQGRFDFS | TGVPDRFTGSGSGTDYTLTISSVQTEDL |
| | AEPSDSTAHLQINNLKNEDTATYFC | ALYYCQQHYNTPYTFGGGTKLEIK (SEQ |
| | ARRSYYGSRGWYFDVWGTGTTVT | ID NO: 294) |
| | VSS (SEQ ID NO: 293) | |
| LK-RB1-7 | QIQLVQSGPELKKPGETVKISCKAS | DVQMIQSPSSLSASLGDIVTMTCQASQD |
| | GYTFTTYGMSWVKQAPGKDLKW | TTINLNWFQQKPGKVPKLLIYGASNLED |
| | MGWINTFSGVPACADDFQGRFDFS | GVPSRFSGSSFGTDFTLTISSLEDEDMAT |
| | AEPSDSTAHLQINNLKNEDTATYFC | YFCLQHTYLPRTFGGGTKLEIR (SEQ ID |
| | ARRSYYGSRGWYFDVWGTGTTVT | NO: 296) |
| | VSS (SEQ ID NO: 295) | |
| LK-RB1-8 | QVQLQQSGAELVRPGTSVKVSCKA | DIVMTQSQKFMSTTVGDRVSITCKASQN |
| | SGYAFSNYLIEWVKQRPGQGLEWI | VGTAVVWYQQKPGQSPKLLIYSASNRY |
| | GVINPGSDGSTYNEKFKGKATLTA | TGVPDRFTGSGSGTDFTLTISNMQSEDL |
| | DKSSNTAYMQLSSLTSEDSAVYFC | ADYFCQQYSSYMYTFGGGTKLEIK |
| | ARGDYGRSYGMDYWGQGTSVTVS | (SEQ ID NO: 298) |
| | S (SEQ ID NO: 297) | |
| LK-RB1-9 | QIQLVQSGPELKKPGETVKISCTAS | DIQMTQSPVSLSASVGETVTITCETSENI |
| | GYTFTTYGMSWVKQAPGKGLKW | YGALNWYQRKQGKSPQLLIYGATNLAD |
| | MGWINTYSGVPTYADDFKGRFAFS | GMSSRFSGSGSGRQYSLEISSLHPDDVAS |
| | LETSASTAYLEINNVKNEDTATYFC | YYCQNVLNTPWTFGGGTKLEIK (SEQ |
| | ARRRRTYHGYLCFDYWGQGTTLT | ID NO: 300) |
| | VSS (SEQ ID NO: 299) | |
| LK-RB1- | QVTLKESGPGILKPSQTLSLTCSFSG | DILLTQSPAILSVSPGERVSFSCRASQSIG |
| 10 | FSLSTSGTGVGWIRQPSGKGLEWL | TNIHWYQQRMNGSPRLIIKYASELISGIP |
| | AHIWWNDDKYYKPSLKRQLTISKD | SRFSGSGSGTDFTLSINSVESEDIADYYC |
| | TSRNQVFLKITSVDTADTATYYCTR | QQSYGWPYTFGGGTKLETK (SEQ ID |
| | TELGRGDYWGQGTSVTVSS (SEQ | NO: 302) |
| | ID NO: 301) | |
| LK-RB1- | QVTLKESGPGILQPSQTLSLTCSFSG | NIVLTQSPASLAVSLGQRATISCRASESV |
| 11 | FSLKTSGMSVGWIRQPSGKGLEWL | DSYGNNFIHWYQQKPGQPPKVLIYYAS |
| | AHIWWNDDKYYNPVLKGRLTISKD | NLEAGVPARFSGSGSRTDFTLTIDPVEAE |
| | TSNNLVFLKIASVVSTDTATYYCVR | DAATYYCQQNNEDPYTFGGGTKLEIE |
| | YGNSHGYWYFDVWGTGTTVTVSS | (SEQ ID NO: 304) |
| | (SEQ ID NO: 303) | |
| LK-RB1- | QVQLQQSGTELVRPGSSVMISCKAS | DIVMTQSHKFMSTSIGDRVSITCKASQD |
| 12 | GYAFSTYWMDWVKQRPGQGLEWI | VSSAVAWYQQKPGQSPKLLIYWASTRH |
| | GQIYPGDGATNYNGKFKGKATLTA | TGVPDRFTGSGSGTDYTLTFSSVQAEDL |
| | DKSSITAYMHLSSLTSEDSAVYFCL | ALYYCQQHYSTPWTFGGGTKLEIK |
| | RSRDYDGWFFDVWGAGTTVTVSS | (SEQ ID NO: 306) |
| | (SEQ ID NO: 305) | |
| LK-RB1- | QVTLKESGPGIVKPSQTLSLTCSFSG | DILLTQSPAILSVSPGDRVSFSCRASQST |
| 13 | FSLSISGTGIGWIRQPSGKGLEWLA | GTNIHWYQRRTNGSPRLLIKYGSESISGI |
| | HIWWDDDKYYNPSLKSQVTISKDT | PSRFSGSGTGTDFTLTINSVESEDIADYY |
| | STKQVFLNITSVDTADTATYYCTRT | CQQSYSWPYTFGGGTKLEIK (SEQ ID |
| | ELGRGDFWGQGTSVTVSS (SEQ ID | NO: 308) |
| | NO: 307) | |
| LK-RB1- | QVTLKESGPGILKPSQTLSLTCSFSG | DILLTQSPAILSVSPGERVSFSCRASQSIG |
| 14 | FSLSTSGMGVGWIRQPSGKGLEWL | TSIHWYQQRINGSPRLLIKYASESISGIPS |
| | AHIWWDDDKYYNPSLKSQLTISKD | RFSGSGSGTDFTLSINSVESEDIADYYCQ |
| | TSRNQVFLKITSVDTADTATYYCAR | QSYSWPYTFGGGTKLEIK (SEQ ID NO: |
| | TELGRGDSWGQGTSVTVSS (SEQ | 310) |
| | ID NO: 309) | |
| LK-RB1- | QVTLKESGPGIVKPSQTLSLTCSFSG | DILLTQSPAILSVSPGDRVSFSCRASQSIG |
| 15 | FSLSISGTGIGWIRQPSGKGLEWLA | TNIHWYQRRTNGSPRLLIKYGSESISGIPS |
| | HIWWDDDKYYNPSLKSQVTISKDT | RFSGSGTGTDFTLTINSVESEDIADYYCQ |
| | STKQVFLNITSVDTADTATYYCTRT | QSYSWPYTFGGGTKLEIK (SEQ ID NO: |
| | ELGRGDFWGQGTSVTVSS (SEQ ID | 312) |
| | NO: 311) | |
| LK-RB1- | QVQLKESGPGLVAPSQSLSITCTVS | DIQMNQSPSSLSASLGDTITITCHASQSV |
| 16 | GFSLISNAISWVRQPPGKGLEWLGV | DFWLNWYQQKPGNIPKLLISKASNLHT |
| | IWGDGNTIYHSALRSRLTISKDNSK | GVPSRFSGSGSGTTFTLTISSLQPEDIATY |
| | SQVFLKLNSLQIDDTATYYCAKAN | YCQQGQSYPLTFGSGTKLEIK (SEQ ID |
| | WDGYYFDYWGQGTTLTVSS (SEQ | NO: 314) |
| | ID NO: 313) | |
| LK-RB1- | QVQLQQPGAELVKPGASVKLSCEA | DIVMTQSQKFMSTSIGDRVSVTCKASQN |
| 17 | SGYTFTNYWIHWVKQRPGRGLEWI | VDTNVAWYQQKPGQSPKALIYSASYRY |
| | GRIAPNSGGTKYNEKFKTRATLTV | SGIPDRFTGSGSGTDFTLTISNVQSEDLA |
| | DKASTTAYMQLSSLTSEDSAVYYC | EYFCQQYNSYPYTFGGGTKLEIK (SEQ |
| | ARLDGSPYYFDYWGQGTTLTVSS | ID NO: 316) |
| | (SEQ ID NO: 315) | |
| LK-RB1- | QVQLKESGPGLVAPSQSLSITCTVS | DIQMTQTPSSLSASLGDRVTISCRASQDI |
| 18 | GFSLTNSGVTWIRQPPGKGLEWLG | SNYLNWYQQKPDGTVKLLIYFTSRLHS |
| | VIWGDGSTNYHSALISRLSISKDNS | GVPSRFSGSGSGTDYSLTISNLEQEDFAT |
| | KSQVFLKLNSLQTDDTATYYCAKP | YFCQQGITLPLTFGAGTKLELK (SEQ ID |
| | HWDYYNMDYWGQGTSVTVSS | NO: 318) |
| | (SEQ ID NO: 317) | |
| LK-RB1- | QVQLKESGPGLVAPSQNLSITCTVS | DIQMTQTTSSLSASLGDRVTISCRASQDI |
| 19 | GFSLTSYGVSWVRQPPGKGLEWLG | SNFLNWYQQKPDGTVKLLIYSTSRLHSG |
| | IIWGDGNTDYHSALISRLSIRKDNSK | VPSRFSGSGSGTDYSLTIANLEQEDVAT |
| | SQVFLKLNSLKSDDTATYYCAKPH | YFCQQGNTLPLTFGAGTKLELK (SEQ ID |
| | WEYYTMDYWGQGTSVTVSS (SEQ | NO: 320) |
| | ID NO: 319) | |
| LK-RB1- | QVQLKESGPGLVAPSQSLSITCTVS | DIQMNQSPSSLSASLGDTITITCHASQNI |
| 20 | GFSLNSNGVSWVRQPPGKGLEWLG | DFWLSWYQQKPGNIPKLLIYKASNLHT |
| | LIWGDGSTNYHSALISRLSIRKDNS | GVPSRFSGSGSGTGFTLTISSLQPEDIATY |
| | KSQVFLKLNSLQNDDTATYYCAKP | YCQQGQSYPLTFGAGTKLELK (SEQ ID |
| | NWDGYHFDYWGQGTTLIVSS (SEQ | NO: 322) |
| | ID NO: 321) | |
| LK-RB1- | QVQLQQPGAELVKPGASVKMSCK | DIVMTQSHKFMSTSIGDRVSITCKASQD |
| 21 | ASGYIFTDYWTTWVKQRPGQGLE | VSTAVAWCQQKPGQSPKSLIYSASYRYT |
| | WIGDIYPVSGSTNYNEKFKNKATLT | GIPDRFTGSGSGTDFTFTISSVQAEDLAV |
| | VDTSSSTAYMQLSSLTSEDSAVYYC | YYCQQHYTNPVTFGTGTKLELK (SEQ |
| | ARALGRRYYFDYWGQGTTLTVSS | ID NO: 324) |
| | (SEQ ID NO: 323) | |
| LK-RB1- | QVQLKESGPGLVAPSQSLSITCTVS | DIQMTQTTSSLSASLGDRVTISCRASQDI |
| 22 | GFSLTSYGVSWVRQPPGKGLEWLG | SNFLNWYQQKPDGTVKLLIYYTSRLHS |
| | IIWGDGNTNYHSALTSRLSISKDNS | GAPSRFSGSGSGTDYSLTISNLDQEDIAT |
| | KSQVFLKLNSLRTDDTATYYCAKP | YFCQQGNTLPLTFGAGTKLELK (SEQ ID |
| | HWDHYSMDYWGQGTSVTVSS | NO: 326) |
| | (SEQ ID NO: 325) | |
| LK-RB1- | QVQLQQTGAELVRPGSSVKLSCKA | DIQMTQSPASQSVSVGETVTITCRASENI |
| 23 | SGYTFTNYWMDWVKQRPGQGLE | YSNLAWYQQKQGKSPQLLVYGATNLA |
| | WIGNIHPFGNETHYNQKFKDKATL | DGVSLRFSGSGSGTQYSLKINSLQSEDFG |
| | TVDKSSSTAYMQLSSLTSEDSAVY | SYYCQHFWGIPWTFGGGTKLEIK (SEQ |
| | YCARGLLLYFDVWGTGTTVTVSS | ID NO: 328) |
| | (SEQ ID NO: 327) | |
| LK-RB1- | QVQLQQPGAELVNPGTSVNLSCKA | DVMMTQTPLTLSVTIGQPASISCKSSQSL |
| 24 | SGYTFTGYWMHWVKQRPGQGLE | LNSDGKTYLNWLLQRPGQSPKRLIYLVS |
| | WIGMIHPNNGGTIYNERFNGKATLT | KLGSGVPDRFTGSGSGTDFTLKISRVEA |
| | VDRSSTTAYIQLSSLTSEDSAVYYC | EDLGVYYCWQGTHFPWTFGGGAKLEIK |
| | AYWDLDYWGQGTTLTVSS (SEQ | (SEQ ID NO: 330) |
| | ID NO: 329) | |
| LK-RB1- | QIQLVQSGPELKKPGETVKISCKAS | DIVMTQSHKFMSTSVGDRVSITCKASQD |
| 25 | GYTFTSYGMNWMKQAPGEGLKW | VNTAVAWYQQKPGQSPKLLIYWASTRH |
| | MGWINTYSGVPTYADDFKGRFAFS | TGVPDRFTGSGSGTDFTLTISSVQAEDLA |
| | LETSATTVYLQINNLKNEDKATYFC | LYYCQQHYTIPFTFGSGTKLEIK (SEQ ID |
| | ARRGRDSVVGHFDVWGTGTTVTV | NO: 332) |
| | SS (SEQ ID NO: 331) | |
| LK-RB1- | QVQLKESGPGLVAPSQSLSITCTVS | DIQMTQTTSSLSASLGDRVTISCRASQDI |
| 26 | GFSLTSYGVSWVRQPPGKGLEWLG | SNFLNWYQQKPDGTFKLLISYTSTLHSG |
| | IIWGDGSTNYHSALISRLSISKDNSK | VPSRFSGSGSGTDYSLTISNLEQEDVATY |
| | SQVFLKLNSLQTDDTATYYCAKPN | FCQQGNTLPLTFGAGTKLALK (SEQ ID |
| | WDYYAMDYWGQGTSVTVSS (SEQ | NO: 334) |
| | ID NO: 333) | |
| LK-RB1- | QVQLQQSGPELVKPGASVRMSCKA | DIQMTQSPASLSVSVGETVTITCRASENI |
| 27 | SGYTFTSHYIHWMKQRPGQGLEWI | YSNLAWYQQKQGKSPHLLVYAATNLA |
| | GWIYPGDGHTKYHEKFKDRATLTA | DGVPSRFSGSGSGTQYSLKINSLQSEDFG |
| | DTSSITAYMRLSSLTSEDSAIYYCV | NYYCQHFWGSPWTFGGGTKLEIK (SEQ |
| | YYSDSWFPYWGQGTLVTVSA (SEQ | ID NO: 336) |
| | ID NO: 335) | |
| LK-RB1- | QVQLQQSGPELVKPGTSVKMSCKA | DIQMTQSPASLSVSVGETVTITCRASENI |
| 28 | SGYTFTSHYIHWMKQRPGQGLDWI | YSNLAWYQQKQGKSPQLLVYAATNLA |
| | GWIYPGDGHTKYHEKFKGKATLTA | DGVPSRFSGSGSGTQYSLKINSLQSEDFG |
| | DTSSITAYMQLSLLTSEDSAVYYCV | NYFCQHFWGSPWTFGGGTKLEIK (SEQ |
| | YYSDSWFPYWGQGTLVTVSA (SEQ | ID NO: 338) |
| | ID NO: 337) | |
| LK-RB1- | QVQLKESGPGLVAPSQNLAITCTVS | DIQMTQTASSLFASLGDRVTISCRASQDI |
| 29 | GFSLTNSGVSWVRQSPGKGLEWLG | SNYLNWYQQKPDGSIKLLISYTSRLHSG |
| | IIWGDGNTNYHSALISRLSISKDNSK | VPSRFSGSGSGTDYSLSISNLEQEDIATY |
| | SQVFLKLNSLQTDDTGTYYCAKPN | FCQQGISLPCTFGGGTKLEVN (SEQ ID |
| | WDYYALDYWGQGTSVTVSS (SEQ | NO: 340) |
| | ID NO: 339) | |
| LK-RB1- | QVQLKESGPGLVAPSQSLSITCTVS | VIQMTQTTSSLSASLGDRVTISCRASQDI |
| 30 | GFSLTSYGVSWIRQPPGKGLEWLG | SNYVNWYQQRPDGTVKLLISYTSRLHS |
| | VIWGDGNTNYHSALISRLSISKDNS | GVPSRFSGSGSGTDYSLTITNLEQEDIAT |
| | KSQVFLKLNSLQTDDTATYYCAKP | YFCQQGITLPCTFGGGTKLEIN (SEQ ID |
| | SWDYYAMDYWGQGTSVTVSS | NO: 342) |
| | (SEQ ID NO: 341) | |
| LK-RB1- | QVQLKESGPGLVAPSQSLSITCTVS | DIQMTQTTSSLSASLGDRVTISCRASQDI |
| 31 | GFSLTSYGVSWVRQPPGKGLEWLG | SNYLNWYLQKPDGTVKLLIYYTSRLHS |
| | IIWGDGSTNYHSALKSRLSISKDNS | GVPSRFSGSGSGTDYSLTISNLEQEDIAT |
| | KSQVFLKLNSLQTEDTATYYCAKP | YFCQQGITLPCTFGGGTKLEIK (SEQ ID |
| | NWDYYAMDYWGQGTSVTVSS | NO: 344) |
| | (SEQ ID NO: 343) | |
| LK-RB1- | QVQLQQSGAELAKPGASVKLSCTA | DIQMTQSPASLSVSVGETVTITCRASENI |
| 32 | SGYTFTTYLMHWVKQRPAQGLEWI | YSSLAWYQQKQGKSPQLLVYAATNLA |
| | GYINPSNDFTKYNQKFKDKATLTA | DGVPSRFSGSGSGTQYSLKINSLQSEDFG |
| | DTSSSTAYMQLNSLTFEDSAVYYC | NYYCQHFWGTWAFGGGTKLEIK (SEQ |
| | ARNSDYDPSYWGQGTTLTVSS | ID NO: 346) |
| | (SEQ ID NO: 345) | |
| LK-RB1- | QIQLVQSGPELKKPGDTVKISCKAS | DIVMTQSHKFMSTSVGDRVSITCKASQD |
| 33 | GYTFTSYGMNWVKQAPGEGLKW | VNTAVAWYQQKPGQSPKLLIYWASTRH |
| | MGWINTYSGVPTYGDDFKGRFAFS | TGVPDRFTGSGSGTDFTLTISSVQAEDLA |
| | LETSASTAYLQINNLENEDTATYFC | LYYCQQHYTIPFTFGSGTRLEIK (SEQ ID |
| | ARRGRDSVVGHFDVWGTGTTVTV | NO: 348) |
| | SS (SEQ ID NO: 347) | |
| LK-RB1- | QVQLQQSGAELVKPGASVKLSCKA | DILLTQSPAILSVSPGERVSFSCRASRYIG |
| 34 | SGYTFTEYTIHWVKQRSGQGLEWI | TSIHWYQQRTNGSPRLLIKYASESISGIPS |
| | GWFYPGSDSIKYNEKFKDKATLTA | RFSGSGSGTDFTLSINSVESEDIADYYCQ |
| | DKSSSTVYMELSRLTSEDSAVYFCS | QSNTWPLTFGAGTKLELK (SEQ ID NO: |
| | RHEGPYFTSQYYFDYWGQGTTLTV | 350) |
| | SS (SEQ ID NO: 349) | |
| LK-RB1- | DVQLQESGPGLEKPSQSLSLTCSVT | DIVLTQSPATLSVTPGDSVSLSCRASQSIS |
| 35 | GYSITRGYQWNWIRQFPGNKLEW | SNLHWYQQKSHESPRLLIKYASQSISGIP |
| | MGYISYDGSNNYNPSLKNRISITRD | SRFSGSGSGTDFTLSINSVETEDFGMYFC |
| | ASKNQFFLKLNSVTTEDTATYYCA | QQSNSWPLTFGAGTKLELK (SEQ ID |
| | RRSYNSIDYWGQGTTLTVSS (SEQ | NO: 352) |
| | ID NO: 351) | |
| LK-RB1- | DVQLQESGPGLEKPSQSLSLTCSVT | DIVLTQSPATLSVTPGDSVSLSCRASQSIS |
| 36 | GYSITRGYQWNWIRQFPGNKLEW | NNLHWYQQKSHESPRLLIKYASQSISGIP |
| | MGYISYDGSNNYNPSLKNRISIIRDT | SRFSGSGSGTDFTLSINSVETEDFGMYFC |
| | SKNQFFLKLNSVTTEDTATYYCAR | QQSNSWPLTFGAGTKLELK (SEQ ID |
| | RSYNSIDYWGQGTTLTVSS (SEQ ID | NO: 354) |
| | NO: 353) | |
| LK-RB1- | QVQLQQPGAELVKPGASVKLSCKA | DILLTQSPAILSVSPGERVSFSCRASQSIG |
| 37 | SGYTFTSYWMHWVKQRPGRGPEW | TSIHWYQQRRNGSPRLLIKYTSESISGIPS |
| | IGRIDPNSGYARYNEKFKNKATLTA | RFSGSGSGTDFTLSINSVESEDIADYYCQ |
| | DKPSSTADMHLSSLTSEDSAVYYCT | QSNSWPHTFGGGTKLELK (SEQ ID NO: |
| | RSFITTAEWFFDVWGTGTTVTVSS | 356) |
| | (SEQ ID NO: 355) | |
| LK-RB1- | DVQLQESGPGLVQPSQSLSLTCSVT | EIVLTQSPATLSVTPGDRVSLSCRASQNI |
| 38 | GYSITSGYQWNWIRQLPGNKLEW | SNYLHWYQQKSHGSPRLIIKYASQSISGI |
| | MAYISFDGSNNYNPSLKNRISITRDT | PSRFSGSGSGTDFTLSINSVETEDFGMYF |
| | SKNQFFLKLKSVTTEDTATYYCAR | CQQSNSWPLTFGAGTKLELK (SEQ ID |
| | RDYDGVAYWGQGTLVTVSA (SEQ | NO: 358) |
| | ID NO: 357) | |
| LK-RB1- | EVQLQQSGAELVRPGASVKLSCTA | QIVLTQSPAIMSASPGEKVTITCSASSSV |
| 39 | SGFNIKDDYVHWVNQRPEQGLEWI | NYMHWFQQKPGSSPKLWIYSTANLASG |
| | GWIDPENGDTGYASKFQDKATITA | VPARFSGSGSGTSYSLTISRMEAEDAAT |
| | DTSSNTAYLHLSSLTSEDTAVYYCT | YYCQQRSSYPPTWTFGGGTKLEIK (SEQ |
| | TTRWTGSYWGQGTTLTVSS (SEQ | ID NO: 360) |
| | ID NO: 359) | |
| LK-RB1- | DVQLQESGPGLVQPSQSLSLTCSVT | DVLMTQTPLSLPVSLGDQASISCRSSQSI |
| 40 | GYSITSGYQWNWIRQLPGNKLEW | VHSNGNTYLEWYLQKPGQSPKLLIYKV |
| | MAYISFDGSNNYNPSLKNRISITRDT | SNRFSGVPDRFSGSGSGTDFTLKISRVEA |
| | SKNQFFLKLKSVTTEDTATYYCAR | EDLGVYYCFQGSHVPWTFGGGTKLEIK |
| | RDYDGVAYWGQGTLVTVSA (SEQ | (SEQ ID NO: 362) |
| | ID NO: 361) | |
| LK-RB1- | DVQLQESGPGLVQPSQSLSLTCSVT | EIVLTQSPATLSVTPGDRVSLSCRASQNI |
| 41 | GYSITSGYQWNWIRQLPGNKLEW | SNYLHWYQQKSHGSPRLIIKYASQSISGI |
| | MAYISFDGSNNYNPSLKNRISITRDT | PSRFSGSGSGTDFTLSINSVETEDFGMYF |
| | SKNQFFLKLKSVTTEDTATYYCAR | CQQSNSWPLTFGAGTKLELK (SEQ ID |
| | RDYDGVAYWGQGTLVTVSA (SEQ | NO: 364) |
| | ID NO: 363) | |
| LK-RB1- | QVQLQQPGAELVKPGTSVKLSCKA | DIVMTQSQKFMSTSVGDRVSVTCKASQ |
| 42 | SGYSFISYWVYWVKQRPGQGLEWI | NVGTNVAWYQQKPGQSPKALIYSASYR |
| | GMIHPNSGSTNFNEKFRYKATLTV | FSGVPDRFTGSGSGTDFTLTIGNVQSEDL |
| | DKSSSTAYIQLSSLTSEDSAVYYCG | AEYFCHQYKNYPLTFGGGTKLEIK (SEQ |
| | RSDDGYPYHAMDSWGQGTSVTVS | ID NO: 366) |
| | S (SEQ ID NO: 365) | |
| LK-RB1- | EFQLQQSGPELMKPGASVKISCKAS | DIQMTQSPGALSVSVGETVTITCRASENI |
| 43 | GYSFTDYNMNWVKQSNGKSLEWI | NSNLAWYQQKQGKSPQLLVYAATNLA |
| | GIIDPNYGTTTYNQKFKGRATLTVD | DGVPSRFSGSGSGTQYSLKINSLQSEDFG |
| | QSSNTAYMQLNSLTSEDSAVYYCA | SYHCQHFWGTPFTFGSGTKLEIK (SEQ |
| | REGRYSWFAYWGQGTLVTVSA | ID NO: 368) |
| | (SEQ ID NO: 367) | |
| LK-RB1- | QVQLQQSGTELAKPGASVKLSCKA | DILLTQSPAILSVSPGERVSFSCRASQSIG |
| 44 | SGYTFTNYWMHWIKQRPGQGLEW | RSIHWFQQRTNGSPSLLIKYASESISGIPS |
| | IGYVHPTSDYTKYNQKFKDKATLT | RFSGGGSGTDFTLSISNVESEDIADYYCQ |
| | ADKSSSTAYMQLNSLTYEDSAVYY | QSNNWPFTFGSGTKLEIK (SEQ ID NO: |
| | CTRSSLYDTNYVFDSWGQGTTLTV | 370) |
| | SS (SEQ ID NO: 369) | |
| LK-RB1- | QVQLQQSGTELARPGASVKLSCKA | DILLTQSPAILSVSPGERVSFSCRASQSIG |
| 45 | SGYTFTNFWMHWIKQRPGQGLEWI | RSIHWYQQRTNGSPRLLIKYASESISGIPS |
| | GYVNPTSDYTKYNQKFKDKATLTA | RFSGGGSGTDFTLSISNVESEDIADYYCQ |
| | DKSSSTAYMQLNSLTYEDSAVYYC | QSNNWPFTFGSGTKLEIK (SEQ ID NO: |
| | TRSSLYDTNYVFDSWGQGTTLTVS | 372) |
| | S (SEQ ID NO: 371) | |
| LK-RB1- | EVQLQQSGPELVKPGASVKISCKTS | DIVLTQSPTSLAVSLGQRAIISCKASQSV |
| 46 | GYTFTDYYMNWVKQSHGKSLEWI | SFAGTSLMHWYQQKPGQQPKLLIYRAS |
| | GDIYPINGDISYNQKFKGKATLTVD | NLKAGVPTRFSGSGSRTDFTLNIHPVEE |
| | KSSSTVYMEIRSLTYEDSAVYYCAR | DDAATYYCQQSREYPMYTFGGGTKLEI |
| | LAQTTSGGFDYWGQGTTLTVSS | K (SEQ ID NO: 374) |
| | (SEQ ID NO: 373) | |
| LK-RB1- | QIQLVQSGPELKKPGETVKISCKAS | DIQMTQTTSSLSASLGDRVTISCRASQDI |
| 47 | GYTFTTYGMSWVKQAPGKGLKW | SNYLNWYQQKPDGTVKLLIYYTSRIHSG |
| | MGWINTYSGVPTYTDDFKGRFAFS | VPSRFSGSGSGTDFSLTISNLDQEDIATY |
| | VETSASTAYLQINNLKNEDTATYFC | FCQQGHTLPWTFGGGTKVEIK (SEQ ID |
| | ARRERDFYGNIFFPYWGQGSLVTV | NO: 376) |
| | SA (SEQ ID NO: 375) | |
| LK-RB1- | EVKLVESGGGLVQPGGSLKLSCAA | DIVMTQSQKFLSTTVGDRVSITCKASQN |
| 48 | SGFTFSDYYMYWVRQTPEKRLEW | VGTAVAWYQQKPGQSPKILIYSASNRYT |
| | VAYISNGGGISFYPDTVKGRFTISRD | GVPDRFTGSGSGTDFTLTISNMQSEDLA |
| | NAKNTLYLQMSRLKSEDTAIYYCA | DYFCQQYSSYRTFGGGTKLEIK (SEQ ID |
| | SATKEVGAMDYWGQGTSVTVSS | NO: 378) |
| | (SEQ ID NO: 377) | |
| LK-RB1- | QIQLVQSGPELKKPGETVKISCKAS | DIVMTQSQKFMSATVGDRVSITCKASQ |
| 49 | GYTFTTYGMSWVKQAPGKGLKW | NVGTAVAWYQQKPGQSPKLLIYSPSNR |
| | MGWINTYSGVPTYADDFKGRFAFS | YTGVPDRFTGSGSGTHFTLTINNMQSED |
| | LETSGSTAYLQISNLKNEDTATYFC | LADYFCQQYSTYPLTFGAGTKLELN |
| | SRLFRSWDSYAMDYWGQGTSVTV | (SEQ ID NO: 380) |
| | SS (SEQ ID NO: 379) | |
| LK-RB1- | QVQLKQSGPGLVQPSQSLSITCTVS | QIVLSQSPAILSAFPGEKVTMTCRASSSV |
| 50 | GFSLTSYGVHWVRQSPGKGLEWLG | SFMHWYQQKPGSSPKPWIYATSNLASG |
| | VIWRGGNTDYNAPFVSRLSITKDNS | VPARFSGSGSGTSYSLTISRVEAEDAATY |
| | QSQVFFKMNSLEADDTAIYYCAKN | YCQQWSSNPLTFGAGTKLELK (SEQ ID |
| | SLDVYSSWYFDVWGTGTTVTVSS | NO: 382) |
| | (SEQ ID NO: 381) | |
| LK-RB1- | QVQIQQSGAELVKPGASVKLSCKA | ENVLTQSPAIMSASLGEKVTMSCRASSS |
| 51 | SGYTFTEFPIHWIKQRSGQGLEWIG | VNYMYWYQQKSDASPKIWIYYTSNLAP |
| | WFYPGSDNMNYNEKFKDKATLTA | GVPARFSGSGSGNSYSLTISSMEGEDAA |
| | DKSSSTVYMELSRLTSEDSAVYFCA | TYYCQQFTSSPFTFGTGTKLEIK (SEQ ID |
| | RHENAYFYAMDYWGQGTSVTVSS | NO: 384) |
| | (SEQ ID NO: 383) | |
| LK-RB1- | EVKLVESGGGLVQPGGSLKLSCAA | DIVMTQSQKFMSTTVGDRVSITCKASQN |
| 52 | SGFIFSDYYMYWVRQTPEKRLEWV | VGTAVAWYQQKPGQSPKLLIYAASNRY |
| | AYISNGGDITYYPDTVKGRFTISRDI | TGVPDRFTGSGSGTDFTLTISNMQSEDL |
| | ARNTLFLQMGRLKSEDTAMYYCAS | ADYFCQQYRSYRTFGGGTNLEIK (SEQ |
| | ATTEVGAMDHWGQGTSVTVSS | ID NO: 386) |
| | (SEQ ID NO: 385) | |
| LK-RB1- | QIQLVQSGPELKKPGETVKISCKAS | DIVMTQSQKFMSTTVGDRVSITCKASQN |
| 53 | GYTFTTYGMSWVNQAPGKGLKW | VGTAVAWYQQKPGQSPKLLIYSASNRY |
| | MGWINTNYGVPTYADDFKGRFAFS | TGVPDRFTGSDSGTDFTLTINNIQSEDLA |
| | LETSASTAYLQINNLKNEDTATYFC | DYFCQQYSSYPLTFATGTKLELK (SEQ |
| | ARGTRRRGISYFAMDYWGQGTSVT | ID NO: 388) |
| | VSS (SEQ ID NO: 387) | |
| LK-RB1- | DVQLQESGPGLVKPSQSLSLTCSVT | ENVLTQSPAIMSASLGEKVTMSCRASSS |
| 54 | GYSITSGFYWNWIRQFPGNKLEWM | VNYMYWYQQKSDASPKLWIYYTSNLA |
| | GHISYDGTNNYNPSLKNRISITRDTS | PGVPVRFSGSGSGNSYSLTISSMEGEDA |
| | KNLFFLKLNSVTTEDTATFHCARG | ATYYCQQFTSSPWTFGGGTKLEIK (SEQ |
| | GYGGSYGFDYWGQGTTLTVSS | ID NO: 390) |
| | (SEQ ID NO: 389) | |
| LK-RB1- | EVKLVESGGGLVQPGGSLKLSCAA | DIVMTQSQKFMSTTVGDRVSITCKASQN |
| 55 | SGFTFSDYYMYWVRQTPEKRLEW | VGTAVAWYQQKPGKSPKLLIYAASNRY |
| | VAYISNGGDITFYPDTVKGRFTISRD | TGVPDRFTGSGSGTDFTLTISNMQSEDL |
| | NARSTLYLQMSRLKSEDTAIYYCAS | ADYFCQQYSSYRTFGGGTNLEIK (SEQ |
| | ATTEVGAMDHWGQGTSVTVSS | ID NO: 392) |
| | (SEQ ID NO: 391) | |
| LK-RB1- | QVQLKQSGPGLVQPSQSLSITCSVS | QIVLSQSPAILSASPGEKVTMTCRASSSV |
| 56 | GFSLTNYGVHWVRQSPGKGLEWL | SFMHWYQQKPGSSPKPWIYATSNLASG |
| | GVIWRGGSTDNNAAFMSRLSITKD | VPARFSGSGSGTSYSLTISRVEAEDAATY |
| | NSKSQVFFKMNSLQADDTAIYYCA | YCQQWSRNPPTFGAGTKLELK (SEQ ID |
| | KNSDFYGSSWYFDVWGTGTTVTVS | NO: 394) |
| | S (SEQ ID NO: 393) | |
| LK-RB1- | EVKLVESGGGLVQPGGSLKLSCAA | DIVMTQSQKFMSTTVGDRVSITCKASQN |
| 57 | SGFTFSDYYMYWVRQTPEKRLEW | VGTAVAWYQQKPGKSPKLLIYAASNRY |
| | VAYISNGGDITFYPDTVKGRFTISRD | TGVPDRFTGSGSGTDFTLTISNVQSEDLA |
| | NARSTLYLQTSRLKSEDTAIYYCAS | DYFCQQYSSYRTFGGGTSLEIK (SEQ ID |
| | ATTEVGAMDHWGQGTSVTVSS | NO: 396) |
| | (SEQ ID NO: 395) | |
| LK-RB1- | QVQLKQSGPGLVQPSQSLSITCTVS | QIVLSQSPAILSASPGEKVTMTCRASSSV |
| 58 | GFSLTTYGVHWVRQSPGKGLEWL | SFMHWYQQKPGSSPKPWIYATSNLASG |
| | GVIWRGGSTDYNAAFMSRLSITKD | VPARFSGSGSGTSYSLTISRVEAEDAATY |
| | NSKSQVFFRMNSLQADDTARYYCA | YCQQWSSNPPTFGGGTKLEIK (SEQ ID |
| | KNSLYYGSSWYFDVWGTGTTVTV | NO: 398) |
| | SS (SEQ ID NO: 397) | |
| LK-RB1- | QIQLVQSGPELKKPGETVKISCKAS | DIQMTQSPASLSVSVGETVTITCRASENI |
| 59 | GYTFTTYGMSWVKQAPGKGLKW | YSNLVWYQQKQGKSPQLLVYTATNLA |
| | MGWINTYSGVPTYADDFKGRFAFS | DGVPSRFSGSGSGTQFSLKINSLQSEDFG |
| | LETSASTAYLQINNLKNEDTATYFC | SYYCQHFWGTPWTFGGGTKLEIK (SEQ |
| | ARRRIYYGNKGYAMDYWGQGTSV | ID NO: 400) |
| | TVSS (SEQ ID NO: 399) | |
| LK-RB2-1 | QIQLVQSGPELKKPGETVKISCKAS | DIVMSQSPSSLAVSVGEKVTMSCKSSQS |
| | GYTFTTYGMSWVKQAPGKGLKW | LLFSSNQKNYLAWYQQKPGQSPKLLIY |
| | MGWINTYSGVSTYADDFKGRFAFS | WASTRESGVPDRFTGRGSGTDFTLTISS |
| | LETSASTAYLQINNLKNEDTATYFC | VKTEDLAVYYCQQYDTYPPTFGGGTKL |
| | ARRYDYDGYAMDYWGQGTSVTVS | EIK (SEQ ID NO: 402) |
| | S (SEQ ID NO: 401) | |
| LK-RB2-2 | DVKLEESGEGLVKPGGSLKLSCAA | DIQMTQSPASLSVSVGETVTITCRASENI |
| | SGFTFSSYAMSWVRQTPEKRLEWV | YSNLAWYQQRQGKSPQLLVYAATNLA |
| | AYISSGGDYFHYADTVKGRFTISRD | DGVPSRFSGSGSGTQYSLKINSLQSEDFG |
| | NARNTLYLQMSSLKSEDTAMYYCT | SYYCQQFWDNPLTFGAGTKLELK (SEQ |
| | REGAHYDSSYLAWFAHWGQGTLV | ID NO: 404) |
| | TVSA (SEQ ID NO: 403) | |
| LK-RB2-3 | QLQLQQSDAELVKPGASVKISCKVS | DIKMTQSPSSMYASLGERVTITCKASQDI |
| | GYSFTDHTIHWMKQRPEQGLEWIG | KSYLSWFQQKPGKSPKTLIYRANRLVD |
| | YIYPRDGSTKENEKFTGKATLTADK | GVPSRFSGSGSGQDYSLTINSLEYEDMEI |
| | SSSTAYMQLTSLTSEDSAVYFCTGS | YYCLQYDEFPYTFGGGTKLEIK (SEQ ID |
| | GFDYWGQGTSLTVSS (SEQ ID NO: | NO: 406) |
| | 405) | |
| LK-RB2-4 | EVQLVESGGDLVKPGGSLKLSCAA | DIQMTQTTSSLSASLGDRVTISCRANQDI |
| | SGFTFSNYGMSWVRQTPDKRLEW | SNYLNWYQQKPDGTVKLLIYYTSRLHS |
| | VATISSGGSYTFYPDSVKGRFTISRD | GVPSRFSGSGSGTDYSLTISDLEQEDIAT |
| | NAKNTLYLQMSSLKSEDTALYYCA | YFCQQGNTLPWTFGGGTKLEIK (SEQ ID |
| | RDYYYGKTIEGYFDYWGQGTTLTV | NO: 408) |
| | SS (SEQ ID NO: 407) | |
| LK-RB2-5 | QIQLVQSGPELKKPGETVKISCKAS | DIVMTQSPATLSVTPGDRVSLSCRASQSI |
| | GYTFTTYGMSWVKQAPGKGLKW | SDYLHWYQQKSHESPRLLIKYASQSISGI |
| | MGWINTYSGVPTYADDFKGRFAFS | PSRFSGSGSGSDFTLSINSVEPEDVGVYY |
| | LETSVSTAYLQINNLKNEDTATYFC | CQNGHGFPWTFGGGTKLEIK (SEQ ID |
| | ARRDGYDGGGNFDYWGQGTTLTV | NO: 410) |
| | SS (SEQ ID NO: 409) | |
| LK-RB2-6 | QIQLVQSGPELKKPGETVKISCKAS | DIVMSQSPSSLAVSVGEKVTVSCKSSQS |
| | GYTFTTYGMSWVKQAPGKGLEWM | LLYSYNQKNYLAWYQQKPGQSPKLLIY |
| | GWINIYSGVPTYADDFKGRFAFSLE | WSSTRESGVPDRFTGSGSGTDFTLTISSV |
| | TSASTAYLQINNLKNEDTATYFCAR | KAEDLAVYYCQQCYNYPYTFGGGTKLE |
| | RGDYDSFYFDYWGQGTTLTVSS | IK (SEQ ID NO: 412) |
| | (SEQ ID NO: 411) | |
| LK-RB2-7 | DVKLVESGEGLVKPGGSLKLSCAA | DIQMTQSPASLSVSVGETVTITCRASENI |
| | SGFTFSSYAMSWVRQTPEMRLEWV | YSNLAWYQQKEGKSPQLLVYGATNLA |
| | AYISSGGDHFYYADTVKGRFTISRD | DGVPSRFSGSGSGTQYSLKINSLQSEDFG |
| | NARSTLYLQMSSLKSEDTAMYYCT | SYYCONFWDIPLTFGAGTKLELK (SEQ |
| | REGASYDSNFLAWFAYWGQGTLV | ID NO: 414) |
| | TVSA (SEQ ID NO: 413) | |
| LK-RB2-8 | QIQLVQSGPELKKPGETVKISCKAS | DIQMTQSPASLSASVGETVTITCRTSENI |
| | GYSFASYGMGWVKQAPGKGLKWL | YSYLAWYQQKQGKSPQLLVYNAKSLA |
| | GWINTHSGVPTYADDFKGRFAFSL | EGVPSRFSGSGSGTQFSLRINSLQPEDFG |
| | ETSASTAYLQINNLKNEDTATYFCA | TYYCQQHYETPLTFGAGTKLELK (SEQ |
| | HYGRPWFPYWGQGTLVTVSA | ID NO: 416) |
| | (SEQ ID NO: 415) | |
| LK-RB2-9 | DVQLQESGPGLVKPSQSLSLTCSVT | DIQMTQSPASLSVSVGETVTITCRASENI |
| | GYSITSGYYWNWIRQFPGNKLEWM | YSNLAWYQQKQGKSPQLLVYGATNLA |
| | GYIGYDGNNNYNPSLKNRISITRDT | DGVPSRFSGRGSDTQYSLKINSLQSEDF |
| | SKNQFFLKLNSLTTEDTATYYCARE | GSYYCQHFWDTPYTFGGGTKLEIK (SEQ |
| | GSTMVTSWFAYWGQGTLVTVSA | ID NO: 418) |
| | (SEQ ID NO: 417) | |
| LK-RB2- | EVMLVESGGDLVKPGGSLKLSCAA | DIVMTQSQKFMSSTGGDRVTITCKASQN |
| 10 | SGFTFSANTVSWLRRTPERRLEWV | VDTAVAWYQQKPGQSPKLLIYSASNRYI |
| | ATISGGSDNTYCPDSVKGRFTISRD | GVPDRFKGSGSGTDFSLTITNIQSEDLAE |
| | NARDTLYLQMSSLTSEDTALYFCA | YFCQQYSSYPLTFGAGTKLELK (SEQ ID |
| | RHYYDGPNWYFDVWGPGTTVTVS | NO: 420) |
| | S (SEQ ID NO: 419) | |
| LK-RB2- | QIQLVQSGPELKKPGETVKISCKAS | DIKMTQSPSSMYASLGERVTITCKASQDI |
| 11 | GYSFITYGMSWVKQAPGKGLKWM | NNYLSWFQQKPGKSPKTLIFRANRLVD |
| | GWINTYSGEPTYGDDFMGRFAFSL | GVPSRFSGSGSGQDYSLTISSLEDEDMGI |
| | ETSASTAYLQINNLKNEDTASYFCA | YYCLQYDEFPWMFGGGTKLEIE (SEQ |
| | RYHYDDGMDYWGQGTSVTVSS | ID NO: 422) |
| | (SEQ ID NO: 421) | |
| LK-RB2- | QIQLVQSGPELKKPGETVKISCKAS | DIVLTQSPASLAVSPGQRATISCRASESV |
| 12 | GYTFTTYGMSWVKQTPGKGLKWM | DTYGISFMHWYQQKPGQPPKLLIYRASN |
| | GWINIYSGVPTYADDFKGRFVFSLE | LESGIPARFSGSGSRTDFTLTINPVETDD |
| | ISASTAHLQINNLKNEDTATYFCAR | VAIYYCQQSKKDPYTFGGGTKLEIK |
| | RKDYSGHCFDYWGQGTTLTVSS | (SEQ ID NO: 424) |
| | (SEQ ID NO: 423) | |
| LK-RB2- | QIQLVQSGPELKKPGETVKISCKAS | DSVMSQSPSSLAVSVGEKVTMSCKSSQS |
| 13 | GYTFTTYGMSWVKQAPGKGLQW | LLYSGNQKSYLAWYQQKPGQSPKLLIY |
| | MGWINTYSGVPTFADDFKGRFAFS | WASTRESGVPDRFTGSGSGTDFTLTISSV |
| | LDTSASTAYLQINNLKNEDTATYFC | KAEDLAVYYCQQYYSYPYTFGGGTKLE |
| | ARRINWDGYAMDYWGQGTSVTVS | IK (SEQ ID NO: 426) |
| | S (SEQ ID NO: 425) | |
| LK-RB2- | QVQLKQSGPGLVQPSQSLSITCTVS | DIQMTQTTSSLSASLGDRVTISCSASQGI |
| 14 | GFSLTSYGVHWVRQSPGKGLEWLG | SNYLNWFQQKPDGTVKLLIYYTSSLHSG |
| | VIWRGGSTDYNAAFMSRLSITKDN | VPSRFSGSGSGTDYSLTISNLEPEDIATY |
| | SKSQVFFKMNSLQADDTAIYYCAK | YCQQYSKLPLTFGAGTKLELK (SEQ ID |
| | NEGDSTVVAWYFDVWGTGTTVTV | NO: 428) |
| | SS (SEQ ID NO: 427) | |
| LK-RB2- | QIQLVQSGPELKKPGETVKISCKAS | DIVLTQSPASLAVSLGQRATFSCRASESV |
| 15 | GYTFTTYGMSWVKQAPGKGLKW | DTYGISFMHWYQQKPGQPPKLLIYRASN |
| | MGWINVYSGVPTYADDFKGRFAFS | LESGIPARFSGSGSRTDFTLTINPVETDD |
| | LEISASTAYLQINNLKDEDTATYFC | VATYYCQQSKKEPYTFGGGTKLEIK |
| | ARRKDYSGHCFDYWGQGTTLTVSS | (SEQ ID NO: 430) |
| | (SEQ ID NO: 429) | |
| LK-RB2- | QIQLVQSGPELKKPGETVKISCKAS | DIQMTQSPASLSASVGETVTITCRASENI |
| 16 | GYTFTTYGMGWVKQAPEKGLKW | YSYLAWYHQKQGESPQLLVYNAKTLAE |
| | MGWINTNSGAPTYGDDFKGRFAFS | GVPSRFSGSGSGTQFSLKINSLQPEDFGS |
| | LETSASTAYLQINNLKNEDTATYFC | YYCQHHYNIPLTFGAGTKLELK (SEQ ID |
| | AHYGRPWFAYWGQGTLVTVSA | NO: 432) |
| | (SEQ ID NO: 431) | |
| LK-RB2- | QIQLVQSGPELKKPGETVKISCKAS | DSVMSQSPSSLAVSVGEKVTLSCKSSQS |
| 17 | GYTFTNYGMSWVKQAPGKGLQW | LLYSGNQKSYLAWYQQKPGQSPKLLIY |
| | MGWINTYSGVPTFADDFRGRFAFS | WASSRESGVPDRFTGSGSGTDFTLTISSV |
| | LDTSASTAYLQINSLKNEDTAAYFC | KAEDLAVYYCQQYYSFPYTFGGGTKLEI |
| | ARRINWDGYAMDYWGQGTSVTVS | K (SEQ ID NO: 434) |
| | S (SEQ ID NO: 433) | |
| LK-RB2- | EVQLQQSGPELVKPGTSVKISCKAS | DIQMTQSPASLSVSVGETVTITCRASENI |
| 18 | GYTFTDYYMNWVKQSHGKSLEWI | YSNLAWYQQKQGKSPQLLVYGATNLA |
| | GDINPNNGGTSYNQKFKGKATLTV | DGVPSRFSGSGSGTQYSLKINSLQSEDFG |
| | DKSSSTAYMELRSLTSEDSAVFYCA | SYYCQHFWDTPFTFGSGTKLEIK (SEQ |
| | RSGTTVVGTYWYFDVWGTGTTVT | ID NO: 436) |
| | VSS (SEQ ID NO: 435) | |
| LK-RB2- | QVQLQQPGAELVKPGASVKLSCKA | QIVLSQSPAILSASPGEKVTMTCRATSRV |
| 19 | SGYTFTSYWMHWVKQRPGQGLEW | SYIHWFQQKPGSSPKPWIYVTSNLASGV |
| | IGMIHPNSGGTNYNEKFKSKATLTV | PARFSGSGSGTSYSLTISKLEAEDVATYY |
| | DKSSSTAYMQFSSLTSEDSAVYYC | CQQWSTNPLTFGAGTKLELK (SEQ ID |
| | ARCTVVALYYALDYWGQGTSVTV | NO: 438) |
| | SS (SEQ ID NO: 437) | |
| LK-RB2- | QVQLKQPGAERVKPGASVKMSCK | NIEMTQSPKSMSMAVGERVTLSCKASE |
| 20 | ASGYTFTRYWITWVKQRPGQGLE | NVGTYVSWYQQKPEQSPKLLIYGASNR |
| | WIGDIYPGSATTNYNEKFKSKATLT | NTGVPDRFTGSGSATDFTLTISSVQAEDL |
| | EDTSSSTAYMQLSSLTSEDSAVYYC | ADYHCGQSYSYPFTFGSGTKLEIK (SEQ |
| | ARSGDYSNSNYAMDYWGQGTSVT | ID NO: 440) |
| | VSS (SEQ ID NO: 439) | |
| LK-RB2- | EVQLVESGGDLVKPGGSLKLSCAA | DIQMNQSPSSLSASLGDTITITCHASQNI |
| 21 | SGFTFSSYGMSWVRQTPDKRLEWV | NVWLSWYQQKPGNIPKLLIYKASNLHT |
| | ATISSGGSYTYYPDSLKGRFTISRDN | GVPSRFSGSGSGTGFTLTISSLQPEDIATY |
| | AKSTLYLQMSSLKSEDTAKYYCAR | YCQQGQSYPLTFGGGTKLEIK (SEQ ID |
| | PYYDYIFYAIDYWGQGTSVTVSS | NO: 442) |
| | (SEQ ID NO: 441) | |
| LK-RB2- | QIQLVQSGPELKKTGETVKISCKAS | NIVMTQTPKFLLVSAGDRVTITCRASQS |
| 22 | GYTFTTYGMSWVKQAPGKGLKW | VTNDVAWYQQRPGQSLKLLIYYASNRY |
| | MGWINIYSGVPTYADDFKGRFAFS | TGVPDRFTGSGYGTDFTFTISTVQAEDL |
| | LETSASTAYLQINNLKNEDTATYFC | AVYFCQQGYSSPFTFGSGTKLEIK (SEQ |
| | ARRADYDGYYFDYWGQGTTLTVS | ID NO: 444) |
| | S (SEQ ID NO: 443) | |
| LK-RB2- | QIQLVQSGPELKKPGETVKISCKAS | NIVLIQSAATLSVTPGDSVSLSCRASQSL |
| 23 | GYTFTTYGMSWVKQAPGKGLKW | SNGLHWYQQKSHESPRLLIKYGSQSISGI |
| | MGWINIYSGVPTYADDFKGRFAFS | PSRFSGSGSGTDFTLSINSVETEDFGMYF |
| | LETSASTAYLQINNLKNEDTATYFC | CQQSDSWPHTFGSGTKLEIK (SEQ ID |
| | ARRGDYDGYYFDYWGQGTTLTVS | NO: 446) |
| | S (SEQ ID NO: 445) | |
| LK-RB2- | QIQLVQSGPEVKKPGETVKISCKAS | DIVMTQSPATLSVTPGDRVSLSCRASQSI |
| 24 | GYTFTTYGMSWVKQAPGKGLKW | SDYLHWYQQKSHESPRLLIKYASQSISGI |
| | MGWLNIYSGVPAYADDFKGRFAFS | PSRFSGSGSGSDFTLSINSVEPEDVGVYY |
| | LESSASTAYLQINNLKNEDTATYFC | CQNGHSFPYTFGGGTKLEIK (SEQ ID |
| | ARRENYDGYGMDYWGQGTSVTVS | NO: 448) |
| | S (SEQ ID NO: 447) | |
| LK-RB2- | QVQLKESGPGLVAPSQSLSITCTVS | DIQMIQSPSSMFASLGDRVSLSCRASQGI |
| 25 | GFSLTSYGVHWVRQPPGKGLEWLG | RGNLDWYQQKPGGTIKLLIYSTSNLNSA |
| | VIWAGGSTKYNSALMSRLSINKDN | VPSRFSGSGSGSDYSLTISNLESEDFADY |
| | SKSQVFLKMNSLQTDDTAMYYCA | YCLQRNASPYTFGGGTKLEIK (SEQ ID |
| | RDQMVRSAMDYWGQGTSVTVSS | NO: 450) |
| | (SEQ ID NO: 449) | |
| LK-RB2- | QVQLKESGPGLVAPSQSLSITCTVS | DIQMIQSPSSMFASLGDRVSLSCRASQGI |
| 26 | GFSLTSYGVHWVRQPPGKGLEWLG | RGNLDWYQQKPGGTIKLLIYSTSNLNSA |
| | VIWAGGSTDYNAAFISRLNISKDISE | VPSRFSGSGSGSDYSLTISNLESEDFADY |
| | SQVFFRMNSLQTDDTAMYYCARD | YCLQRNASPYTFGGGTKLEIK (SEQ ID |
| | QMVRSAMDYWGQGTSVTVSS | NO: 452) |
| | (SEQ ID NO: 451) | |
| LK-RB2- | DVKLVESGGGLVKPGGSLKLSCAA | DILMTQSPSSMSVSLGDTVSITCHASQDI |
| 27 | SGFTFSSYSMSWVRQTPEKRLEWV | SSNIGWLQQKPGNSFKGLIYHGTNLEDG |
| | ATISTGGSYTYYPDSVKGRFTISRD | VPSRFSGSGSGADYSLTISSLESEDFADY |
| | NAKNTLYLQMSSLKSEDTAMFYCT | YCVQYAQFPYTFGGGTKLEIK (SEQ ID |
| | RGSMDYWGQGTSVTVSS (SEQ ID | NO: 454) |
| | NO: 453) | |
| LK-RB2- | DVKLVESGGGLVKPGGSLKLSCAA | DILMTQSPSSMSVSLGDTVSIACHASQDI |
| 28 | SGFTFSSYSMSWVRQTPEKRLEWV | SSNIGWLQQKPGNSFKGLIYHGTNLEDG |
| | ATISTGGSYTYYPDSMKGRFTISRD | VPSRFSGSGSGADYSLTISSLESEDFADY |
| | NAKNTLYLQMSSLKSEDTAMFYCT | YCVQYAQFPYTFGGGTKLEIK (SEQ ID |
| | RGSMDYWGQGTSVTVSS (SEQ ID | NO: 456) |
| | NO: 455) | |
| LK-RB2- | QVQLQQSGAELARPGASVKLSCKA | DIQMTQSPASLSASVGETVTITCRASGNI |
| 29 | SGYTFTSYWMQWVKQRPGQGLEW | HNYLAWYQQKQGKSPQLLVYNAKTLA |
| | IGAIYPGDGDTRYTQKFKGKATLTA | DGVPSRFSGSGSGTQYSLKINSLQPEDFG |
| | DKSSSTAYMQLSSLASEDSAVYYC | SYYCQHFWSTPPTFGGGTKLEIK (SEQ |
| | ARGYYGSSYADYFDYWGQGTTLT | ID NO: 458) |
| | VSS (SEQ ID NO: 457) | |
| LK-RB2- | EVQLVESGGGLVQPGGSLKLSCAA | DIQMTQSPASLSVSVGETVTITCRASENI |
| 30 | SGFTFNTDGMSWVRQTPDKRLELV | NNNLAWYHQKQGKSPQLLVYAATNLA |
| | AIINSNGGSTYYPDSVKGRFTISRDN | DGVPSRFSGSGSGTQYSLKINSLQSEDFG |
| | AKNTLYLQMSSLKSEDTAMYYCA | SYYCQHFWGTPFTFGGGTKLEIK (SEQ |
| | REGSYGSWFAYWGQGTLVTVSA | ID NO: 460) |
| | (SEQ ID NO: 459) | |
| LK-RB2- | QVQLQQPGAELVKPGASMKLSCKA | SIVMTQTPEFLLVSAGDRVTITCKASQSV |
| 31 | SGYTFTTYYMYWVKQRPGLGLEWI | NNDVAWFQQKPGQSPKLLIYYASNRYT |
| | GGINPSNGGTTFNEKFKNKATLTVD | GVPDRFTGSGYGTDFTFTISTVQAEDLAI |
| | KSSSTAYMQLRSLTSEDSAVYYCT | YFCQQDYSSPPTFGGATKLEIK (SEQ ID |
| | RWRFFDVWGAGTTVTVSS (SEQ ID | NO: 462) |
| | NO: 461) | |
| LK-RB2- | DVQLQESGPGLVKPSQSLSLTCSVT | DIVLTQSPVTLSVTPGDSVSLSCRASQNI |
| 32 | GYSITSGYYWNWIRQFPGNKLEWM | NKNLHWYQQRSHESPRLLIKYVSQSISGI |
| | GYISFDGSNHYNPSLRNRISITRDTF | PSRFSGSGSGTDFTLSINSVETEDFGMYF |
| | KNQFFLKLNSVTTEDTATFYCTRRD | CQQSNSWPLTFGAGTKLELR (SEQ ID |
| | YNWFAYWGQGTLVTVSA (SEQ ID | NO: 464) |
| | NO: 463) | |
| LK-RB2- | EVQLQQSGVELVRPGALVKLSCKA | DVVMTQSHKFMSTSVGDRVTITCKASQ |
| 33 | SGFNIKDYYMHWVKQRPEQGLEWI | DVSNAVAWYQQKPGQSPKLLIYSASYR |
| | GWIDPEKGDTIYGPKFQGKASITAD | YTGVPDRFTGSGSGTDFTFTISSMQAED |
| | TSSNTAYLQLSSLTSEDTAVYYCSR | LAVYYCQQHYSLPWTFGGGTKLEIK |
| | GPNYGSSYAFAYWGQGTLVTVSA | (SEQ ID NO: 466) |
| | (SEQ ID NO: 465) | |
| LK-RB2- | DVKVVESGGGLVKPGGSLKLSCAA | DILMTQSPSSMSVSLGDTVSITCHASQDI |
| 34 | SGFTFSSYSMSWVRQTPEKRLEWV | SSNIGWLQQKPGNSFKGLIYHGINLEDG |
| | ATISTGSSYTYYSDSVKGRFTISRDN | VPSRFSGSGSGADYSLTISSLESEDFADY |
| | AKNTLYLQMSSLKSEDTAMFYCTR | YCVQYAQFPYTFGGGTKLEIK (SEQ ID |
| | GSMDYWGQGTSVTVSS (SEQ ID | NO: 468) |
| | NO: 467) | |
| LK-RB2- | EVQLQQSGPELVKPGASVKISCKAS | DIVMTQSPATLSVTPGDRVSLSCRASQSI |
| 35 | GYTFTDYNMHWVKQSHGKSLEWI | SDYLHWYQQKSHESPRLLIKYASQSISGI |
| | GSIYPYNGGSDYNQKFKSKATLTV | PSRFSGSGSGSDFTLSINSVEPEDVGVYY |
| | DNSSSTAYMELRSLTSEDSAVYYC | CQTGHSFPYTFGGGTKLEIK (SEQ ID |
| | ASELTGLYAMDYWGQGTSVTVSS | NO: 470) |
| | (SEQ ID NO: 469) | |
| LK-RB2- | QVQLQQSGAELVRPGASVKMSCK | DIVLTQSPASLSVTPGDSVSLSCRASQSIS |
| 36 | ASGYTFTNYWIGWAKQRPGHGLE | NNLHWYQQKSHESPRLLIKYASQSISGIP |
| | WIGDIYPGGTYTGYNEKFKGKATL | SRFSGSGSGTDFTLSINSVETEDFGMYFC |
| | TADKSSSTAYMQFSSLTSEDSAIYY | QQSNSWPLTFGAGTKLELK (SEQ ID |
| | CARRMDYDGYAMDYWGQGTSVT | NO: 472) |
| | VSS (SEQ ID NO: 471) | |
| LK-RB2- | QIQLVQSGPEVKKPGETVKISCKAS | DIVMSQSPSSLAVSVGEKVTMSCKSSQS |
| 37 | GYTFTTYGMSWVKQAPGKGLKW | LLYSGNQKNYLAWYQQKPGQSPKLLIY |
| | MGWINTYSGVPTYADDFKGRFAFS | WASARESGVPDRFTGSGSGTDFTLTISS |
| | LETSVSTAYLQINNLKNEDTSTYFC | VKAEDLAVYYCQQYYTYPFTFGSGTKL |
| | ARRRDYDGYAMDYWGQGTSVTVS | EIK (SEQ ID NO: 474) |
| | S (SEQ ID NO: 473) | |
| LK-RB2- | EVKLVESGGGLVQPGGSLKLSCAA | DIVMTQSHTFMSTSVGDRVSITCKASQD |
| 38 | SGFSFSDYYMYWVRQTPEKRLEW | VSTAVAWYQQKPGQSPKLLIYWASTRH |
| | VAYISNGGGNTYYPDTVKGRFTISR | TGVPDRFTGSGSGTDYTLTISSVQAEDL |
| | DNAKNSLYLQMSRLKSEDTGMYY | ALYYCQQHYSTPRTFGGGTKLEIK (SEQ |
| | CARDREDWYFDVWGTGTTVTVSS | ID NO: 476) |
| | (SEQ ID NO: 475) | |
| LK-RB2- | DVKLVESGGGSVQPGGSLKLSCAA | DIVMTQSPSSLAMSVGQKVTMSCKSSQS |
| 39 | SGFTFSDYYMYWVRQTPEKRLEW | LLHSSNQKNYLAWYQQKPGQSPKLLVS |
| | VAYISNGGGNTYYPDTVKGRFTISR | FASTRESGVPDRFIGSGSGTDFTLTINSV |
| | DNAKNTLYLQMSRLKSEDTAMYY | QAEDLADYFCQQHYSTPLTFGAGTKLE |
| | CARSNWAWYFDVWGTGTTVTVSS | LK (SEQ ID NO: 478) |
| | (SEQ ID NO: 477) | |
| LK-RB2- | DVQLQESGPGLVKPSQSLSLTCSVT | DIQMTQSPASLSVSVGETVTITCRASENI |
| 40 | GYSITSGYYWNWIRQFPGNKLEWM | YSNLAWYQQKQGKSPQLLVYAATNLA |
| | AYISYDGSNNYNPSLKNRISITRDTS | DGVPSRFSGSGSGTQYSLKINSLQSEDFG |
| | KNQFFLKLNSVTTEDTATYYCARE | SYYCQHFWDTPFTFGSGTKLEIK (SEQ |
| | EGTAQATLGYFDYWGQGTTLTVSS | ID NO: 480) |
| | (SEQ ID NO: 479) | |
| LK-RB2- | EVQLQLSGPELVKPGASVKMSCKA | DIVMTQSHKFMSTSVGDRVSITCKASQD |
| 41 | SGYTFTDYNMHWVKQSHGKSLEW | VSTAVAWYQQKPGQSPKLLIYSASYRY |
| | IGYINPNNGGTTYNQKFKGKATLT | TGVPDRFTGSGSGTDFTFTISSVQAEDLA |
| | VNKSSSTAYMELRSLTSEDSAVYY | IYYCQQHYSIPWTFGGGTKLEIK (SEQ |
| | CARAPYYGSSYLLAYWGQGTLVT | ID NO: 482) |
| | VSA (SEQ ID NO: 481) | |
| LK-RB2- | EFQLQQSGPELVKPGASVKISCKAS | DVLMTQTPLSLPVSLGDQASISCRSSQSI |
| 42 | GYSFTDYNMNWVKQSNGKSLDWI | VHSNGNTYLEWYLQKPGQPPKLLIYKV |
| | GIINPKYGTTAYNQKLRDKATLTV | SNRFSGVPERFSGSGSGTDFTLKINRVEA |
| | DQSSSTAYMQLNSLTSEDSAVYYC | EDLGVYYCFQGSLVPPTFGGGTKLEIK |
| | ASLNWDEGFAYWGQGTLVTVSP | (SEQ ID NO: 484) |
| | (SEQ ID NO: 483) | |
| LK-RB2- | EVQLVESGGDLVKPGGSLKLSCAA | QIVLTQSPAILSASPGEKVTMTCSANSSV |
| 43 | SGFTFSSYGMSWVRQTPDKRLEWV | SSMYKYQQKPGSSPKPWIYGTSNLASG |
| | ATISGGGTYTYYPDSVKGRFTISRD | VPARFSGSGSGTSYSLTISSMEAADAAT |
| | NAKNTLYLQMSSLKSEDTAMYYC | YYCQQYHSHPLTFGAGTKLELK (SEQ |
| | ASGAMDYWGQGTSVTVSS (SEQ | ID NO: 486) |
| | ID NO: 485) | |
| LK-RB2- | EVQLVESGGDLVKPGGSLKLSCAA | DVVMTQTPLTLSVTIGQPASISCKSSQSL |
| 44 | SGFTFSSYGMSWVRQTPDKRLEWV | LDIDGKTFLNWLLQRPGQSPKRLIYLVS |
| | ATISGGGTYTYYPDSVKGRFTISRD | KLDSGVPDRFTGSGSGTDFTLKISRVEA |
| | NAKNTLYLQMSSLKSEDTAMYYC | EDLGVYYCWQGTHFPWTFGGGTKLEIK |
| | ASGAMDYWGQGTSVTVSS (SEQ | (SEQ ID NO: 488) |
| | ID NO: 487) | |
| LK-RB2- | QIQLVQSGPELKKPGETVKISCKAS | DIVMTQSPATLSVTPGDRVSLSCRASQSI |
| 45 | GYTFTTYGMSWVKQTPGKGLQWM | SDYLNWYQQKSHESPRLLIKYVSQSISGI |
| | GWINVYSGVPTYADDFKGRFAFSL | PSRFSGSGSGSDFTLSINSVEPEDIGVYY |
| | EASASTAYLQINNLKNEDTASYFCA | CQNGHNFPLTFGGGTKLELK (SEQ ID |
| | RRGDYDGHYFDYWGQGTTLTVSS | NO: 490) |
| | (SEQ ID NO: 489) | |
| LK-RB2- | QVQMQQSDAELVKPGASVKISCKV | DIVMTQSHKFMSASVGDRVSITCKASQD |
| 46 | SGYTFTDQIIHWMKQRPEQGLEWI | VNTAVAWYRQDPGQSPKLLIYWASTRH |
| | GYIYPRDDSIMYNEKFKGKATLTA | TGVPDRFTGSGSGTDYTLTINNFQAEDL |
| | DKSSSTANMQLNSLTSEDSAVYFC | ALYYCQQHYSPPLTFGAGTKLELK (SEQ |
| | AYDAYWYFDVWGTGTTVIVSS | ID NO: 492) |
| | (SEQ ID NO: 491) | |
| LK-RB2- | QVQLKQSGPGLVQPSQSLSITCTVS | DIQMTQTTSSLSASLGDRVIISCRASQDI |
| 47 | GFSLISYGVHWVRQSPGKGLEWLG | NNYLNWYQQKPDGTVKLLIYFTSRLHS |
| | VIWSGGSTDYNAAFISRLSISKDNS | GVPSSFSGSGSGTDYSLTINNLEQEDIAT |
| | KSQVFFKMNSLQADDTAIYYCARS | YFCQQVNTLPYTFGGGTKLEIK (SEQ ID |
| | FHYHGSTFDYWGQGTTLTVSS | NO: 494) |
| | (SEQ ID NO: 493) | |

TABLE 6
CDR sequences of anti-LILRB1 (LK-RB1-1 to LK-RB1-59) and anti-LILRB2
LK-RB2-1 to LK-RB2-47) antibodies

| Antibody | CDR-H1 | CDR-H2 | CDR-H3 | CDR-L1 | CDR-L2 | CDR-L3 |
|---|---|---|---|---|---|---|
| LK-RB1-1 | NFGMS | WINTYSG | RRRTFDG | TLSSQHST | LKTDGSH | GVGDTIK |
| | (SEQ ID | VPTYADD | YLCFDY | YSID (SEQ | RTGD | EQFVYV |
| | NO: 495) | FKG (SEQ | (SEQ ID | ID NO: | (SEQ ID | (SEQ ID |
| | | ID NO: | NO: 497) | 498) | NO: 499) | NO: 500) |
| | | 496) | | | | |
| LK-RB1-2 | TYGVS | WINTYSG | RRRTYDG | GASENIY | GATNLAD | QNVLTTP |
| | (SEQ ID | VSTYSDD | YLSFDY | GALN | (SEQ ID | WT (SEQ |
| | NO: 501) | FKG (SEQ | (SEQ ID | (SEQ ID | NO: 505) | ID NO: |
| | | ID NO: | NO: 503) | NO: 504) | | 506) |
| | | 502) | | | | |
| LK-RB1-3 | SYWIH | RINPYSSG | FDGAPDY | KASQNVG | SASYRYS | QQYNNYP |
| | (SEQ ID | TRYNEKF | FDF (SEQ | INVA (SEQ | (SEQ ID | FT (SEQ |
| | NO: 507) | KN (SEQ | ID NO: | ID NO: | NO: 511) | ID NO: |
| | | ID NO: | 509) | 510) | | 512) |
| | | 508) | | | | |
| LK-RB1-4 | SYWMH | RIDPHYSG | FDGDPYY | KASQNVG | SASYRYS | QQYNNNP |
| | (SEQ ID | TKYNEKF | FDY (SEQ | TNVA | (SEQ ID | FT (SEQ |
| | NO: 513) | KN (SEQ | ID NO: | (SEQ ID | NO: 517) | ID NO: |
| | | ID NO: | 515) | NO: 516) | | 518) |
| | | 514) | | | | |
| LK-RB1-5 | DFWIH | MIHPNSGS | SGDGYSN | RASSSVSY | ATSNLAS | QQWRRSP |
| | (SEQ ID | TNFNEKF | PMDY | MH (SEQ | (SEQ ID | YT (SEQ |
| | NO: 519) | RA (SEQ | (SEQ ID | ID NO: | NO: 523) | ID NO: |
| | | ID NO: | NO: 521) | 522) | | 524) |
| | | 520) | | | | |
| LK-RB1-6 | TYGMS | WINTFSG | RSYYGSR | TASEDVN | WASTRHT | QQHYNTP |
| | (SEQ ID | VPACADD | GWYFDV | TAVA | (SEQ ID | YT (SEQ |
| | NO: 525) | FQG (SEQ | (SEQ ID | (SEQ ID | NO: 529) | ID NO: |
| | | ID NO: | NO: 527) | NO: 528) | | 530) |
| | | 526) | | | | |
| LK-RB1-7 | TYGMS | WINTFSG | RSYYGSR | QASQDTTI | GASNLED | LQHTYLP |
| | (SEQ ID | VPACADD | GWYFDV | NLN (SEQ | (SEQ ID | RT (SEQ |
| | NO: 531) | FQG (SEQ | (SEQ ID | ID NO: | NO: 535) | ID NO: |
| | | ID NO: | NO: 533) | 534) | | 536) |
| | | 532) | | | | |
| LK-RB1-8 | NYLIE | VINPGSDG | GDYGRSY | KASQNVG | SASNRYT | QQYSSYM |
| | (SEQ ID | STYNEKF | GMDY | TAVV | (SEQ ID | YT (SEQ |
| | NO: 537) | KG (SEQ | (SEQ ID | (SEQ ID | NO: 541) | ID NO: |
| | | ID NO: | NO: 539) | NO: 540) | | 542) |
| | | 538) | | | | |
| LK-RB1-9 | TYGMS | WINTYSG | RRRTYHG | ETSENIYG | GATNLAD | QNVLNTP |
| | (SEQ ID | VPTYADD | YLCFDY | ALN (SEQ | (SEQ ID | WT (SEQ |
| | NO: 543) | FKG (SEQ | (SEQ ID | ID NO: | NO: 547) | ID NO: |
| | | ID NO: | NO: 545) | 546) | | 548) |
| | | 544) | | | | |
| LK-RB1- | TSGTGVG | HIWWND | TELGRGD | RASQSIGT | YASELIS | QQSYGWP |
| 10 | (SEQ ID | DKYYKPS | Y (SEQ ID | NIH (SEQ | (SEQ ID | YT (SEQ |
| | NO: 549) | LKR (SEQ | NO: 551) | ID NO: | NO: 553) | ID NO: |
| | | ID NO: | | 552) | | 554) |
| | | 550) | | | | |
| LK-RB1- | TSGMSVG | HIWWND | YGNSHGY | RASESVD | YASNLEA | QQNNEDP |
| 11 | (SEQ ID | DKYYNPV | WYFDV | SYGNNFIH | (SEQ ID | YT (SEQ |
| | NO: 555) | LKG (SEQ | (SEQ ID | (SEQ ID | NO: 559) | ID NO: |
| | | ID NO: | NO: 557) | NO: 558) | | 560) |
| | | 556) | | | | |
| LK-RB1- | TYWMD | QIYPGDG | SRDYDGW | KASQDVS | WASTRHT | QQHYSTP |
| 12 | (SEQ ID | ATNYNGK | FFDV | SAVA | (SEQ ID | WT (SEQ |
| | NO: 561) | FKG (SEQ | (SEQ ID | (SEQ ID | NO: 565) | ID NO: |
| | | ID NO: | NO: 563) | NO: 564) | | 566) |
| | | 562) | | | | |
| LK-RB1- | ISGTGIG | HIWWDD | TELGRGD | RASQSTG | YGSESIS | QQSYSWP |
| 13 | (SEQ ID | DKYYNPS | F (SEQ ID | TNIH (SEQ | (SEQ ID | YT (SEQ |
| | NO: 567) | LKS (SEQ | NO: 569) | ID NO: | NO: 571) | ID NO: |
| | | ID NO: | | 570) | | 572) |
| | | 568) | | | | |
| LK-RB1- | TSGMGVG | HIWWDD | TELGRGD | RASQSIGT | YASESIS | QQSYSWP |
| 14 | (SEQ ID | DKYYNPS | S (SEQ ID | SIH (SEQ | (SEQ ID | YT (SEQ |
| | NO: 573) | LKS (SEQ | NO: 575) | ID NO: | NO: 577) | ID NO: |
| | | ID NO: | | 576) | | 578) |
| | | 574) | | | | |
| LK-RB1- | ISGTGIG | HIWWDD | TELGRGD | RASQSIGT | YGSESIS | QQSYSWP |
| 15 | (SEQ ID | DKYYNPS | F (SEQ ID | NIH (SEQ | (SEQ ID | YT (SEQ |
| | NO: 579) | LKS (SEQ | NO: 581) | ID NO: | NO: 583) | ID NO: |
| | | ID NO: | | 582) | | 584) |
| | | 580) | | | | |
| LK-RB1- | SNAIS | VIWGDGN | ANWDGY | HASQSVD | KASNLHT | QQGQSYP |
| 16 | (SEQ ID | TIYHSALR | YFDY | FWLN | (SEQ ID | LT (SEQ |
| | NO: 585) | S (SEQ ID | (SEQ ID | (SEQ ID | NO: 589) | ID NO: |
| | | NO: 586) | NO: 587) | NO: 588) | | 590) |
| LK-RB1- | NYWIH | RIAPNSGG | LDGSPYY | KASQNVD | SASYRYS | QQYNSYP |
| 17 | (SEQ ID | TKYNEKF | FDY (SEQ | TNVA | (SEQ ID | YT (SEQ |
| | NO: 591) | KT (SEQ | ID NO: | (SEQ ID | NO: 595) | ID NO: |
| | | ID NO: | 593) | NO: 594) | | 596) |
| | | 592) | | | | |
| LK-RB1- | NSGVT | VIWGDGS | PHWDYY | RASQDISN | FTSRLHS | QQGITLPL |
| 18 | (SEQ ID | TNYHSALI | NMDY | YLN (SEQ | (SEQ ID | T (SEQ ID |
| | NO: 597) | S (SEQ ID | (SEQ ID | ID NO: | NO: 601) | NO: 602) |
| | | NO: 598) | NO: 599) | 600) | | |
| LK-RB1- | SYGVS | IIWGDGN | PHWEYYT | RASQDISN | STSRLHS | QQGNTLP |
| 19 | (SEQ ID | TDYHSALI | MDY (SEQ | FLN (SEQ | (SEQ ID | LT (SEQ |
| | NO: 603) | S (SEQ ID | ID NO: | ID NO: | NO: 607) | ID NO: |
| | | NO: 604) | 605) | 606) | | 608) |
| LK-RB1- | SNGVS | LIWGDGS | PNWDGY | HASQNIDF | KASNLHT | QQGQSYP |
| 20 | (SEQ ID | TNYHSALI | HFDY | WLS (SEQ | (SEQ ID | LT (SEQ |
| | NO: 609) | S (SEQ ID | (SEQ ID | ID NO: | NO: 613) | ID NO: |
| | | NO: 610) | NO: 611) | 612) | | 614) |
| LK-RB1- | DYWTT | DIYPVSGS | ALGRRYY | KASQDVS | SASYRYT | QQHYTNP |
| 21 | (SEQ ID | TNYNEKF | FDY (SEQ | TAVA | (SEQ ID | VT (SEQ |
| | NO: 615) | KN (SEQ | ID NO: | (SEQ ID | NO: 619) | ID NO: |
| | | ID NO: | 617) | NO: 618) | | 620) |
| | | 616) | | | | |
| LK-RB1- | SYGVS | IIWGDGN | PHWDHYS | RASQDISN | YTSRLHS | QQGNTLP |
| 22 | (SEQ ID | TNYHSAL | MDY (SEQ | FLN (SEQ | (SEQ ID | LT (SEQ |
| | NO: 621) | TS (SEQ | ID NO: | ID NO: | NO: 625) | ID NO: |
| | | ID NO: | 623) | 624) | | 626) |
| | | 622) | | | | |
| LK-RB1- | NYWMD | NIHPFGNE | GLLLYFD | RASENIYS | GATNLAD | QHFWGIP |
| 23 | (SEQ ID | THYNQKF | V (SEQ ID | NLA (SEQ | (SEQ ID | WT (SEQ |
| | NO: 627) | KD (SEQ | NO: 629) | ID NO: | NO: 631) | ID NO: |
| | | ID NO: | | 630) | | 632) |
| | | 628) | | | | |
| LK-RB1- | GYWMH | MIHPNNG | WDLDY | KSSQSLLN | LVSKLGS | WQGTHFP |
| 24 | (SEQ ID | GTIYNERF | (SEQ ID | SDGKTYL | (SEQ ID | WT (SEQ |
| | NO: 633) | NG (SEQ | NO: 635) | N (SEQ ID | NO: 637) | ID NO: |
| | | ID NO: | | NO: 636) | | 638) |
| | | 634) | | | | |
| LK-RB1- | SYGMN | WINTYSG | RGRDSVV | KASQDVN | WASTRHT | QQHYTIPF |
| 25 | (SEQ ID | VPTYADD | GHFDV | TAVA | (SEQ ID | T (SEQ ID |
| | NO: 639) | FKG (SEQ | (SEQ ID | (SEQ ID | NO: 643) | NO: 644) |
| | | ID NO: | NO: 641) | NO: 642) | | |
| | | 640) | | | | |
| LK-RB1- | SYGVS | IIWGDGST | PNWDYY | RASQDISN | YTSTLHS | QQGNTLP |
| 26 | (SEQ ID | NYHSALIS | AMDY | FLN (SEQ | (SEQ ID | LT (SEQ |
| | NO: 645) | (SEQ ID | (SEQ ID | ID NO: | NO: 649) | ID NO: |
| | | NO: 646) | NO: 647) | 648) | | 650) |
| LK-RB1- | SHYIH | WIYPGDG | YSDSWFP | RASENIYS | AATNLAD | QHFWGSP |
| 27 | (SEQ ID | HTKYHEK | Y (SEQ ID | NLA (SEQ | (SEQ ID | WT (SEQ |
| | NO: 651) | FKD (SEQ | NO: 653) | ID NO: | NO: 655) | ID NO: |
| | | ID NO: | | 654) | | 656) |
| | | 652) | | | | |
| LK-RB1- | SHYIH | WIYPGDG | YSDSWFP | RASENIYS | AATNLAD | QHFWGSP |
| 28 | (SEQ ID | HTKYHEK | Y (SEQ ID | NLA (SEQ | (SEQ ID | WT (SEQ |
| | NO: 657) | FKG (SEQ | NO: 659) | ID NO: | NO: 661) | ID NO: |
| | | ID NO: | | 660) | | 662) |
| | | 658) | | | | |
| LK-RB1- | NSGVS | IIWGDGN | PNWDYY | RASQDISN | YTSRLHS | QQGISLPC |
| 29 | (SEQ ID | TNYHSALI | ALDY | YLN (SEQ | (SEQ ID | T (SEQ ID |
| | NO: 663) | S (SEQ ID | (SEQ ID | ID NO: | NO: 667) | NO: 668) |
| | | NO: 664) | NO: 665) | 666) | | |
| LK-RB1- | SYGVS | VIWGDGN | PSWDYYA | RASQDISN | YTSRLHS | QQGITLPC |
| 30 | (SEQ ID | TNYHSALI | MDY (SEQ | YVN (SEQ | (SEQ ID | T (SEQ ID |
| | NO: 669) | S (SEQ ID | ID NO: | ID NO: | NO: 673) | NO: 674) |
| | | NO: 670) | 671) | 672) | | |
| LK-RB1- | SYGVS | IIWGDGST | PNWDYY | RASQDISN | YTSRLHS | QQGITLPC |
| 31 | (SEQ ID | NYHSALK | AMDY | YLN (SEQ | (SEQ ID | T (SEQ ID |
| | NO: 675) | S (SEQ ID | (SEQ ID | ID NO: | NO: 679) | NO: 680) |
| | | NO: 676) | NO: 677) | 678) | | |
| LK-RB1- | TYLMH | YINPSNDF | NSDYDPS | RASENIYS | AATNLAD | QHFWGT |
| 32 | (SEQ ID | TKYNQKF | Y (SEQ ID | SLA (SEQ | (SEQ ID | WA (SEQ |
| | NO: 681) | KD (SEQ | NO: 683) | ID NO: | NO: 685) | ID NO: |
| | | ID NO: | | 684) | | 686) |
| | | 682) | | | | |
| LK-RB1- | SYGMN | WINTYSG | RGRDSVV | KASQDVN | WASTRHT | QQHYTIPF |
| 33 | (SEQ ID | VPTYGDD | GHFDV | TAVA | (SEQ ID | T (SEQ ID |
| | NO: 687) | FKG (SEQ | (SEQ ID | (SEQ ID | NO: 691) | NO: 692) |
| | | ID NO: | NO: 689) | NO: 690) | | |
| | | 688) | | | | |
| LK-RB1- | EYTIH | WFYPGSD | HEGPYFTS | RASRYIGT | YASESIS | QQSNTWP |
| 34 | (SEQ ID | SIKYNEKF | QYYFDY | SIH (SEQ | (SEQ ID | LT (SEQ |
| | NO: 693) | KD (SEQ | (SEQ ID | ID NO: | NO:697) | ID NO: |
| | | ID NO: | NO: 695) | 696) | | 698) |
| | | 694) | | | | |
| LK-RB1- | RGYQWN | YISYDGSN | RSYNSIDY | RASQSISS | YASQSIS | QQSNSWP |
| 35 | (SEQ ID | NYNPSLK | (SEQ ID | NLH (SEQ | (SEQ ID | LT (SEQ |
| | NO: 699) | N (SEQ ID | NO: 701) | ID NO: | NO: 703) | ID NO: |
| | | NO: 700) | | 702) | | 704) |
| LK-RB1- | RGYQWN | YISYDGSN | RSYNSIDY | RASQSISN | YASQSIS | QQSNSWP |
| 36 | (SEQ ID | NYNPSLK | (SEQ ID | NLH (SEQ | (SEQ ID | LT (SEQ |
| | NO: 705) | N (SEQ ID | NO: 707) | ID NO: | NO: 709) | ID NO: |
| | | NO: 706) | | 708) | | 710) |
| LK-RB1- | SYWMH | RIDPNSGY | SFITTAEW | RASQSIGT | YTSESIS | QQSNSWP |
| 37 | (SEQ ID | ARYNEKF | FFDV | SIH (SEQ | (SEQ ID | HT (SEQ |
| | NO: 711) | KN (SEQ | (SEQ ID | ID NO: | NO: 715) | ID NO: |
| | | ID NO: | NO: 713) | 714) | | 716) |
| | | 712) | | | | |
| LK-RB1- | SGYQWN | YISFDGSN | RDYDGVA | RASQNISN | YASQSIS | QQSNSWP |
| 38 | (SEQ ID | NYNPSLK | Y (SEQ ID | YLH (SEQ | (SEQ ID | LT (SEQ |
| | NO: 717) | N (SEQ ID | NO: 719) | ID NO: | NO: 721) | ID NO: |
| | | NO: 718) | | 720) | | 722) |
| LK-RB1- | DDYVH | WIDPENG | TRWTGSY | SASSSVN | STANLAS | QQRSSYPP |
| 39 | (SEQ ID | DTGYASK | (SEQ ID | YMH (SEQ | (SEQ ID | TWT (SEQ |
| | NO: 723) | FQD (SEQ | NO: 725) | ID NO: | NO: 727) | ID NO: |
| | | ID NO: | | 726) | | 728) |
| | | 724) | | | | |
| LK-RB1- | SGYQWN | YISFDGSN | RDYDGVA | RSSQSIVH | KVSNRFS | FQGSHVP |
| 40 | (SEQ ID | NYNPSLK | Y (SEQ ID | SNGNTYL | (SEQ ID | WT (SEQ |
| | NO: 729) | N (SEQ ID | NO: 731) | E (SEQ ID | NO: 733) | ID NO: |
| | | NO: 730) | | NO: 732) | | 734) |
| LK-RB1- | SGYQWN | YISFDGSN | RDYDGVA | RASQNISN | YASQSIS | QQSNSWP |
| 41 | (SEQ ID | NYNPSLK | Y (SEQ ID | YLH (SEQ | (SEQ ID | LT (SEQ |
| | NO: 735) | N (SEQ ID | NO: 737) | ID NO: | NO: 739) | ID NO: |
| | | NO: 736) | | 738) | | 740) |
| LK-RB1- | SYWVY | MIHPNSGS | SDDGYPY | KASQNVG | SASYRFS | HQYKNYP |
| 42 | (SEQ ID | TNFNEKF | HAMDS | TNVA | (SEQ ID | LT (SEQ |
| | NO: 741) | RY (SEQ | (SEQ ID | (SEQ ID | NO: 745) | ID NO: |
| | | ID NO: | NO: 743) | NO: 744) | | 746) |
| | | 742) | | | | |
| LK-RB1- | DYNMN | IIDPNYGT | EGRYSWF | RASENINS | AATNLAD | QHFWGTP |
| 43 | (SEQ ID | TTYNQKF | AY (SEQ | NLA (SEQ | (SEQ ID | FT (SEQ |
| | NO: 747) | KG (SEQ | ID NO: | ID NO: | NO: 751) | ID NO: |
| | | ID NO: | 749) | 750) | | 752) |
| | | 748) | | | | |
| LK-RB1- | NYWMH | YVHPTSD | SSLYDTN | RASQSIGR | YASESIS | QQSNNWP |
| 44 | (SEQ ID | YTKYNQK | YVFDS | SIH (SEQ | (SEQ ID | FT (SEQ |
| | NO: 753) | FKD (SEQ | (SEQ ID | ID NO: | NO: 757) | ID NO: |
| | | ID NO: | NO: 755) | 756) | | 758) |
| | | 754) | | | | |
| LK-RB1- | NFWMH | YVNPTSD | SSLYDTN | RASQSIGR | YASESIS | QQSNNWP |
| 45 | (SEQ ID | YTKYNQK | YVFDS | SIH (SEQ | (SEQ ID | FT (SEQ |
| | NO: 759) | FKD (SEQ | (SEQ ID | ID NO: | NO: 763) | ID NO: |
| | | ID NO: | NO: 761) | 762) | | 764) |
| | | 760) | | | | |
| LK-RB1- | DYYMN | DIYPINGD | LAQTTSG | KASQSVS | RASNLKA | QQSREYP |
| 46 | (SEQ ID | ISYNQKFK | GFDY | FAGTSLM | (SEQ ID | MYT (SEQ |
| | NO: 765) | G (SEQ ID | (SEQ ID | H (SEQ ID | NO: 769) | ID NO: |
| | | NO: 766) | NO: 767) | NO: 768) | | 770) |
| LK-RB1- | TYGMS | WINTYSG | RERDFYG | RASQDISN | YTSRIHS | QQGHTLP |
| 47 | (SEQ ID | VPTYTDD | NIFFPY | YLN (SEQ | (SEQ ID | WT (SEQ |
| | NO: 771) | FKG (SEQ | (SEQ ID | ID NO: | NO: 775) | ID NO: |
| | | ID NO: | NO: 773) | 774) | | 776) |
| | | 772) | | | | |
| LK-RB1- | DYYMY | YISNGGGI | ATKEVGA | KASQNVG | SASNRYT | QQYSSYR |
| 48 | (SEQ ID | SFYPDTV | MDY (SEQ | TAVA | (SEQ ID | T (SEQ ID |
| | NO: 777) | KG (SEQ | ID NO: | (SEQ ID | NO: 781) | NO: 782) |
| | | ID NO: | 779) | NO: 780) | | |
| | | 778) | | | | |
| LK-RB1- | TYGMS | WINTYSG | LFRSWDS | KASQNVG | SPSNRYT | QQYSTYP |
| 49 | (SEQ ID | VPTYADD | YAMDY | TAVA | (SEQ ID | LT (SEQ |
| | NO: 783) | FKG (SEQ | (SEQ ID | (SEQ ID | NO: 787) | ID NO: |
| | | ID NO: | NO: 785) | NO: 786) | | 788) |
| | | 784) | | | | |
| LK-RB1- | SYGVH | VIWRGGN | NSLDVYS | RASSSVSF | ATSNLAS | QQWSSNP |
| 50 | (SEQ ID | TDYNAPF | SWYFDV | MH (SEQ | (SEQ ID | LT (SEQ |
| | NO: 789) | VS (SEQ | (SEQ ID | ID NO: | NO: 793) | ID NO: |
| | | ID NO: | NO: 791) | 792) | | 794) |
| | | 790) | | | | |
| LK-RB1- | EFPIH | WFYPGSD | HENAYFY | RASSSVN | YTSNLAP | QQFTSSPF |
| 51 | (SEQ ID | NMNYNE | AMDY | YMY (SEQ | (SEQ ID | T (SEQ ID |
| | NO: 795) | KFKD | (SEQ ID | ID NO: | NO: 799) | NO: 800) |
| | | (SEQ ID | NO: 797) | 798) | | |
| | | NO: 796) | | | | |
| LK-RB1- | DYYMY | YISNGGDI | ATTEVGA | KASQNVG | AASNRYT | QQYRSYR |
| 52 | (SEQ ID | TYYPDTV | MDH (SEQ | TAVA | (SEQ ID | T (SEQ ID |
| | NO: 801) | KG (SEQ | ID NO: | (SEQ ID | NO: 805) | NO: 806) |
| | | ID NO: | 803) | NO: 804) | | |
| | | 802) | | | | |
| LK-RB1- | TYGMS | WINTNYG | GTRRRGIS | KASQNVG | SASNRYT | QQYSSYP |
| 53 | (SEQ ID | VPTYADD | YFAMDY | TAVA | (SEQ ID | LT (SEQ |
| | NO: 807) | FKG (SEQ | (SEQ ID | (SEQ ID | NO: 811) | ID NO: |
| | | ID NO: | NO: 809) | NO: 810) | | 812) |
| | | 808) | | | | |
| LK-RB1- | SGFYWN | HISYDGT | GGYGGSY | RASSSVN | YTSNLAP | QQFTSSP |
| 54 | (SEQ ID | NNYNPSL | GFDY | YMY (SEQ | (SEQ ID | WT (SEQ |
| | NO: 813) | KN (SEQ | (SEQ ID | ID NO: | NO: 817) | ID NO: |
| | | ID NO: | NO: 815) | 816) | | 818) |
| | | 814) | | | | |
| LK-RB1- | DYYMY | YISNGGDI | ATTEVGA | KASQNVG | AASNRYT | QQYSSYR |
| 55 | (SEQ ID | TFYPDTV | MDH (SEQ | TAVA | (SEQ ID | T (SEQ ID |
| | NO: 819) | KG (SEQ | ID NO: | (SEQ ID | NO: 823) | NO: 824) |
| | | ID NO: | 821) | NO: 822) | | |
| | | 820) | | | | |
| LK-RB1- | NYGVH | VIWRGGS | NSDFYGS | RASSSVSF | ATSNLAS | QQWSRNP |
| 56 | (SEQ ID | TDNNAAF | SWYFDV | MH (SEQ | (SEQ ID | PT (SEQ |
| | NO: 825) | MS (SEQ | (SEQ ID | ID NO: | NO: 829) | ID NO: |
| | | ID NO: | NO: 827) | 828) | | 830) |
| | | 826) | | | | |
| LK-RB1- | DYYMY | YISNGGDI | ATTEVGA | KASQNVG | AASNRYT | QQYSSYR |
| 57 | (SEQ ID | TFYPDTV | MDH (SEQ | TAVA | (SEQ ID | T (SEQ ID |
| | NO: 831) | KG (SEQ | ID NO: | (SEQ ID | NO: 835) | NO: 836) |
| | | ID NO: | 833) | NO: 834) | | |
| | | 832) | | | | |
| LK-RB1- | TYGVH | VIWRGGS | NSLYYGS | KASQNVG | AASNRYT | QQYSSYR |
| 58 | (SEQ ID | TDYNAAF | SWYFDV | TAVA | (SEQ ID | T (SEQ ID |
| | NO: 837) | MS (SEQ | (SEQ ID | (SEQ ID | NO: 841) | NO: 842) |
| | | ID NO: | NO: 839) | NO: 840) | | |
| | | 838) | | | | |
| LK-RB1- | TYGMS | WINTYSG | RRIYYGN | RASENIYS | TATNLAD | QHFWGTP |
| 59 | (SEQ ID | VPTYADD | KGYAMD | NLV (SEQ | (SEQ ID | WT (SEQ |
| | NO: 843) | FKG (SEQ | Y (SEQ ID | ID NO: | NO: 847) | ID NO: |
| | | ID NO: | NO: 845) | 846) | | 848) |
| | | 844) | | | | |
| LK-RB2-1 | TYGMS | WINTYSG | RYDYDGY | KSSQSLLF | WASTRES | QQYDTYP |
| | (SEQ ID | VSTYADD | AMDY | SSNQKNY | (SEQ ID | PT (SEQ |
| | NO: 849) | FKG (SEQ | (SEQ ID | LA (SEQ | NO: 853) | ID NO: |
| | | ID NO: | NO: 851) | ID NO: | | 854) |
| | | 850) | | 852) | | |
| LK-RB2-2 | SYAMS | YISSGGDY | EGAHYDS | RASENIYS | AATNLAD | QQFWDNP |
| | (SEQ ID | FHYADTV | SYLAWFA | NLA (SEQ | (SEQ ID | LT (SEQ |
| | NO: 855) | KG (SEQ | H (SEQ ID | ID NO: | NO: 859) | ID NO: |
| | | ID NO: | NO: 857) | 858) | | 860) |
| | | 856) | | | | |
| LK-RB2-3 | DHTIH | YIYPRDGS | SGFDY | KASQDIKS | RANRLVD | LQYDEFP |
| | (SEQ ID | TKENEKF | (SEQ ID | YLS (SEQ | (SEQ ID | YT (SEQ |
| | NO: 861) | TG (SEQ | NO: 863) | ID NO: | NO: 865) | ID NO: |
| | | ID NO: | | 864) | | 866) |
| | | 862) | | | | |
| LK-RB2-4 | NYGMS | TISSGGSY | DYYYGKT | RANQDIS | YTSRLHS | YTSRLHS |
| | (SEQ ID | TFYPDSV | IEGYFDY | NYLN | (SEQ ID | (SEQ ID |
| | NO: 867) | KG (SEQ | (SEQ ID | (SEQ ID | NO: 871) | NO: 872) |
| | | ID NO: | NO: 869) | NO: 870) | | |
| | | 868) | | | | |
| LK-RB2-5 | TYGMS | WINTYSG | RDGYDGG | RASQSISD | YASQSIS | QNGHGFP |
| | (SEQ ID | VPTYADD | GNFDY | YLH (SEQ | (SEQ ID | WT (SEQ |
| | NO: 873) | FKG (SEQ | (SEQ ID | ID NO: | NO: 877) | ID NO: |
| | | ID NO: | NO: 875) | 876) | | 878) |
| | | 874) | | | | |
| LK-RB2-6 | TYGMS | WINIYSGV | RGDYDSF | KSSQSLLY | WSSTRES | QQCYNYP |
| | (SEQ ID | PTYADDF | YFDY | SYNQKNY | (SEQ ID | YT (SEQ |
| | NO: 879) | KG (SEQ | (SEQ ID | LA (SEQ | NO: 883) | ID NO: |
| | | ID NO: | NO: 881) | ID NO: | | 884) |
| | | 880) | | 882) | | |
| LK-RB2-7 | SYAMS | YISSGGDH | EGASYDS | RASENIYS | GATNLAD | QNFWDIP |
| | (SEQ ID | FYYADTV | NFLAWFA | NLA (SEQ | (SEQ ID | LT (SEQ |
| | NO: 885) | KG (SEQ | Y (SEQ ID | ID NO: | NO: 889) | ID NO: |
| | | ID NO: | NO: 887) | 888) | | 890) |
| | | 886) | | | | |
| LK-RB2-8 | SYGMG | WINTHSG | YGRPWFP | RTSENIYS | NAKSLAE | QQHYETP |
| | (SEQ ID | VPTYADD | Y (SEQ ID | YLA (SEQ | (SEQ ID | LT (SEQ |
| | NO: 891) | FKG (SEQ | NO: 893) | ID NO: | NO: 895) | ID NO: |
| | | ID NO: | | 894) | | 896) |
| | | 892) | | | | |
| LK-RB2-9 | SGYYWN | YIGYDGN | EGSTMVT | RASENIYS | GATNLAD | QHFWDTP |
| | (SEQ ID | NNYNPSL | SWFAY | NLA (SEQ | (SEQ ID | YT (SEQ |
| | NO: 897) | KN (SEQ | (SEQ ID | ID NO: | NO: 901) | ID NO: |
| | | ID NO: | NO: 899) | 900) | | 902) |
| | | 898) | | | | |
| LK-RB2- | ANTVS | TISGGSDN | HYYDGPN | KASQNVD | SASNRYI | QQYSSYP |
| 10 | (SEQ ID | TYCPDSV | WYFDV | TAVA | (SEQ ID | LT (SEQ |
| | NO: 903) | KG (SEQ | (SEQ ID | (SEQ ID | NO: 907) | ID NO: |
| | | ID NO: | NO: 905) | NO: 906) | | 908) |
| | | 904) | | | | |
| LK-RB2- | TYGMS | WINTYSG | YHYDDG | KASQDIN | RANRLVD | LQYDEFP |
| 11 | (SEQ ID | EPTYGDD | MDY (SEQ | NYLS | (SEQ ID | WM (SEQ |
| | NO: 909) | FMG (SEQ | ID NO: | (SEQ ID | NO: 913) | ID NO: |
| | | ID NO: | 911) | NO: 912) | | 914) |
| | | 910) | | | | |
| LK-RB2- | TYGMS | WINIYSGV | RKDYSGH | RASESVD | RASNLES | QQSKKDP |
| 12 | (SEQ ID | PTYADDF | CFDY | TYGISFM | (SEQ ID | YT (SEQ |
| | NO: 915) | KG (SEQ | (SEQ ID | H (SEQ ID | NO: 919) | ID NO: |
| | | ID NO: | NO: 917) | NO: 918) | | 920) |
| | | 916) | | | | |
| LK-RB2- | TYGMS | WINTYSG | RINWDGY | KSSQSLLY | WASTRES | QQYYSYP |
| 13 | (SEQ ID | VPTFADD | AMDY | SGNQKSY | (SEQ ID | YT (SEQ |
| | NO: 921) | FKG (SEQ | (SEQ ID | LA (SEQ | NO: 925) | ID NO: |
| | | ID NO: | NO: 923) | ID NO: | | 926) |
| | | 922) | | 924) | | |
| LK-RB2- | SYGVH | VIWRGGS | NEGDSTV | SASQGISN | YTSSLHS | QQYSKLP |
| 14 | (SEQ ID | TDYNAAF | VAWYFD | YLN (SEQ | (SEQ ID | LT (SEQ |
| | NO: 927) | MS (SEQ | V (SEQ ID | ID NO: | NO: 931) | ID NO: |
| | | ID NO: | NO: 929) | 930) | | 932) |
| | | 928) | | | | |
| LK-RB2- | TYGMS | WINVYSG | RKDYSGH | RASESVD | RASNLES | QQSKKEP |
| 15 | (SEQ ID | VPTYADD | CFDY | TYGISFM | (SEQ ID | YT (SEQ |
| | NO: 933) | FKG (SEQ | (SEQ ID | H (SEQ ID | NO: 937) | ID NO: |
| | | ID NO: | NO: 935) | NO: 936) | | 938) |
| | | 934) | | | | |
| LK-RB2- | TYGMG | WINTNSG | YGRPWFA | RASENIYS | NAKTLAE | QHHYNIP |
| 16 | (SEQ ID | APTYGDD | Y (SEQ ID | YLA (SEQ | (SEQ ID | LT (SEQ |
| | NO: 939) | FKG (SEQ | NO: 941) | ID NO: | NO: 943) | ID NO: |
| | | ID NO: | | 942) | | 944) |
| | | 940) | | | | |
| LK-RB2- | NYGMS | WINTYSG | RINWDGY | KSSQSLLY | WASSRES | QQYYSFP |
| 17 | (SEQ ID | VPTFADD | AMDY | SGNQKSY | (SEQ ID | YT (SEQ |
| | NO: 945) | FRG (SEQ | (SEQ ID | LA (SEQ | NO: 949) | ID NO: |
| | | ID NO: | NO: 947) | ID NO: | | 950) |
| | | 946) | | 948) | | |
| LK-RB2- | DYYMN | DINPNNG | SGTTVVG | RASENIYS | GATNLAD | QHFWDTP |
| 18 | (SEQ ID | GTSYNQK | TYWYFDV | NLA (SEQ | (SEQ ID | FT (SEQ |
| | NO: 951) | FKG (SEQ | (SEQ ID | ID NO: | NO: 955) | ID NO: |
| | | ID NO: | NO: 953) | 954) | | 956) |
| | | 952) | | | | |
| LK-RB2- | SYWMH | MIHPNSG | CTVVALY | RATSRVS | VTSNLAS | QQWSTNP |
| 19 | (SEQ ID | GTNYNEK | YALDY | YIH (SEQ | (SEQ ID | LT (SEQ |
| | NO: 957) | FKS (SEQ | (SEQ ID | ID NO: | NO: 961) | ID NO: |
| | | ID NO: | NO: 959) | 960) | | 962) |
| | | 958) | | | | |
| LK-RB2- | RYWIT | DIYPGSAT | SGDYSNS | KASENVG | GASNRNT | GQSYSYP |
| 20 | (SEQ ID | TNYNEKF | NYAMDY | TYVS | (SEQ ID | FT (SEQ |
| | NO: 963) | KS (SEQ | (SEQ ID | (SEQ ID | NO: 967) | ID NO: |
| | | ID NO: | NO: 965) | NO: 966) | | 968) |
| | | 964) | | | | |
| LK-RB2- | SYGMS | TISSGGSY | PYYDYIFY | HASQNIN | KASNLHT | QQGQSYP |
| 21 | (SEQ ID | TYYPDSL | AIDY (SEQ | VWLS | (SEQ ID | LT (SEQ |
| | NO: 969) | KG (SEQ | ID NO: | (SEQ ID | NO: 973) | ID NO: |
| | | ID NO: | 971) | NO: 972) | | 974) |
| | | 970) | | | | |
| LK-RB2- | TYGMS | WINIYSGV | RADYDGY | RASQSVT | YASNRYT | QQGYSSP |
| 22 | (SEQ ID | PTYADDF | YFDY | NDVA | (SEQ ID | FT (SEQ |
| | NO: 975) | KG (SEQ | (SEQ ID | (SEQ ID | NO: 979) | ID NO: |
| | | ID NO: | NO: 977) | NO: 978) | | 980) |
| | | 976) | | | | |
| LK-RB2- | TYGMS | WINIYSGV | RGDYDGY | RASQSLS | YGSQSIS | QQSDSWP |
| 23 | (SEQ ID | PTYADDF | YFDY | NGLH | (SEQ ID | HT (SEQ |
| | NO: 981) | KG (SEQ | (SEQ ID | (SEQ ID | NO: 985) | ID NO: |
| | | ID NO: | NO: 983) | NO: 984) | | 986) |
| | | 982) | | | | |
| LK-RB2- | TYGMS | WLNIYSG | RENYDGY | RASQSISD | YASQSIS | QNGHSFP |
| 24 | (SEQ ID | VPAYADD | GMDY | YLH (SEQ | (SEQ ID | YT (SEQ |
| | NO: 987) | FKG (SEQ | (SEQ ID | ID NO: | NO: 991) | ID NO: |
| | | ID NO: | NO: 989) | 990) | | 992) |
| | | 988) | | | | |
| LK-RB2- | SYGVH | VIWAGGS | DQMVRSA | RASQGIR | STSNLNS | LORNASP |
| 25 | (SEQ ID | TKYNSAL | MDY (SEQ | GNLD | (SEQ ID | YT (SEQ |
| | NO: 993) | MS (SEQ | ID NO: | (SEQ ID | NO: 997) | ID NO: |
| | | ID NO: | 995) | NO: 996) | | 998) |
| | | 994) | | | | |
| LK-RB2- | SYGVH | VIWAGGS | DQMVRSA | RASQGIR | STSNLNS | LORNASP |
| 26 | (SEQ ID | TDYNAAF | MDY (SEQ | GNLD | (SEQ ID | YT (SEQ |
| | NO: 999) | IS (SEQ ID | ID NO: | (SEQ ID | NO: 1003) | ID NO: |
| | | NO: 1000) | 1001) | NO: 1002) | | 1004) |
| LK-RB2- | SYSMS | TISTGGSY | GSMDY | HASQDISS | HGTNLED | VQYAQFP |
| 27 | (SEQ ID | TYYPDSV | (SEQ ID | NIG (SEQ | (SEQ ID | YT (SEQ |
| | NO: 1005) | KG (SEQ | NO: 1007) | ID NO: | NO: 1009) | ID NO: |
| | | ID NO: | | 1008) | | 1010) |
| | | 1006) | | | | |
| LK-RB2- | SYSMS | TISTGGSY | GSMDY | HASQDISS | HGTNLED | VQYAQFP |
| 28 | (SEQ ID | TYYPDSM | (SEQ ID | NIG (SEQ | (SEQ ID | YT (SEQ |
| | NO: 1011) | KG (SEQ | NO: 1013) | ID NO: | NO: 1015) | ID NO: |
| | | ID NO: | | 1014) | | 1016) |
| | | 1012) | | | | |
| LK-RB2- | SYWMQ | AIYPGDG | GYYGSSY | RASGNIH | NAKTLAD | QHFWSTP |
| 29 | (SEQ ID | DTRYTQK | ADYFDY | NYLA | (SEQ ID | PT (SEQ |
| | NO: 1017) | FKG (SEQ | (SEQ ID | (SEQ ID | NO: 1021) | ID NO: |
| | | ID | NO: 1019) | NO: 1020) | | 1022) |
| | | NO:1018) | | | | |
| LK-RB2- | TDGMS | IINSNGGS | EGSYGSW | RASENINN | AATNLAD | QHFWGTP |
| 30 | (SEQ ID | TYYPDSV | FAY (SEQ | NLA (SEQ | (SEQ ID | FT (SEQ |
| | NO: 1023) | KG (SEQ | ID NO: | ID NO: | NO: 1027) | ID NO: |
| | | ID NO: | 1025) | 1026) | | 1028) |
| | | 1024) | | | | |
| LK-RB2- | TYYMY | GINPSNGG | WRFFDV | KASQSVN | YASNRYT | QQDYSSP |
| 31 | (SEQ ID | TTFNEKF | (SEQ ID | NDVA | (SEQ ID | PT (SEQ |
| | NO: 1029) | KN (SEQ | NO: 1031) | (SEQ ID | NO: 1033) | ID NO: |
| | | ID NO: | | NO: 1032) | | 1034) |
| | | 1030) | | | | |
| LK-RB2- | SGYYWN | YISFDGSN | RDYNWFA | RASQNIN | YVSQSIS | QQSNSWP |
| 32 | (SEQ ID | HYNPSLR | Y (SEQ ID | KNLH | (SEQ ID | LT (SEQ |
| | NO: 1035) | N (SEQ ID | NO: 1037) | (SEQ ID | NO: 1039) | ID NO: |
| | | NO: 1036) | | NO: 1038) | | 1040) |
| LK-RB2- | DYYMH | WIDPEKG | GPNYGSS | KASQDVS | SASYRYT | QQHYSLP |
| 33 | (SEQ ID | DTIYGPKF | YAFAY | NAVA | (SEQ ID | WT (SEQ |
| | NO: 1041) | QG (SEQ | (SEQ ID | (SEQ ID | NO: 1045) | ID NO: |
| | | ID NO: | NO: 1043) | NO: 1044) | | 1046) |
| | | 1042) | | | | |
| LK-RB2- | SYSMS | TISTGSSY | GSMDY | HASQDISS | HGINLED | VQYAQFP |
| 34 | (SEQ ID | TYYSDSV | (SEQ ID | NIG (SEQ | (SEQ ID | YT (SEQ |
| | NO: 1047) | KG (SEQ | NO: 1049) | ID NO: | NO: 1051) | ID NO: |
| | | ID NO: | | 1050) | | 1052) |
| | | 1048) | | | | |
| LK-RB2- | DYNMH | SIYPYNGG | ELTGLYA | RASQSISD | YASQSIS | QTGHSFP |
| 35 | (SEQ ID | SDYNQKF | MDY (SEQ | YLH (SEQ | (SEQ ID | YT (SEQ |
| | NO: 1053) | KS (SEQ | ID NO: | ID NO: | NO: 1057) | ID NO: |
| | | ID NO: | 1055) | 1056) | | 1058) |
| | | 1054) | | | | |
| LK-RB2- | NYWIG | DIYPGGT | RMDYDG | RASQSISN | YASQSIS | QQSNSWP |
| 36 | (SEQ ID | YTGYNEK | YAMDY | NLH (SEQ | (SEQ ID | LT (SEQ |
| | NO: 1059) | FKG (SEQ | (SEQ ID | ID NO: | NO: 1063) | ID NO: |
| | | ID NO: | NO: 1061) | 1062) | | 1064) |
| | | 1060) | | | | |
| LK-RB2- | TYGMS | WINTYSG | RRDYDGY | KSSQSLLY | WASARES | QQYYTYP |
| 37 | (SEQ ID | VPTYADD | AMDY | SGNQKNY | (SEQ ID | FT (SEQ |
| | NO: 1065) | FKG (SEQ | (SEQ ID | LA (SEQ | NO: 1069) | ID NO: |
| | | ID NO: | NO: 1067) | ID NO: | | 1070) |
| | | 1066) | | 1068) | | |
| LK-RB2- | DYYMY | YISNGGG | DREDWYF | KASQDVS | WASTRHT | QQHYSTP |
| 38 | (SEQ ID | NTYYPDT | DV (SEQ | TAVA | (SEQ ID | RT (SEQ |
| | NO: 1071) | VKG (SEQ | ID NO: | (SEQ ID | NO: 1075) | ID NO: |
| | | ID NO: | 1073) | NO: 1074) | | 1076) |
| | | 1072) | | | | |
| LK-RB2- | DYYMY | YISNGGG | SNWAWY | KSSQSLLH | FASTRES | QQHYSTP |
| 39 | (SEQ ID | NTYYPDT | FDV (SEQ | SSNQKNY | (SEQ ID | LT (SEQ |
| | NO: 1077) | VKG (SEQ | ID NO: | LA (SEQ | NO: 1081) | ID NO: |
| | | ID NO: | 1079) | ID NO: | | 1082) |
| | | 1078) | | 1080) | | |
| LK-RB2- | SGYYWN | YISYDGSN | EEGTAQA | RASENIYS | AATNLAD | QHFWDTP |
| 40 | (SEQ ID | NYNPSLK | TLGYFDY | NLA (SEQ | (SEQ ID | FT (SEQ |
| | NO: 1083) | N (SEQ ID | (SEQ ID | ID NO: | NO: 1087) | ID NO: |
| | | NO: 1084) | NO: 1085) | 1086) | | 1088) |
| LK-RB2- | DYNMH | YINPNNG | APYYGSS | KASQDVS | SASYRYT | QQHYSIP |
| 41 | (SEQ ID | GTTYNQK | YLLAY | TAVA | (SEQ ID | WT (SEQ |
| | NO: 1089) | FKG (SEQ | (SEQ ID | (SEQ ID | NO: 1093) | ID NO: |
| | | ID NO: | NO: 1091) | NO: 1092) | | 1094) |
| | | 1090) | | | | |
| LK-RB2- | DYNMN | IINPKYGT | LNWDEGF | RSSQSIVH | KVSNRFS | FQGSLVPP |
| 42 | (SEQ ID | TAYNQKL | AY (SEQ | SNGNTYL | (SEQ ID | T (SEQ ID |
| | NO: 1095) | RD (SEQ | ID NO: | E (SEQ ID | NO: 1099) | NO: 1100) |
| | | ID NO: | 1097) | NO: 1098) | | |
| | | 1096) | | | | |
| LK-RB2- | SYGMS | TISGGGTY | GAMDY | SANSSVSS | GTSNLAS | QQYHSHP |
| 43 | (SEQ ID | TYYPDSV | (SEQ ID | MY (SEQ | (SEQ ID | LT (SEQ |
| | NO: 1101) | KG (SEQ | NO: 1103) | ID NO: | NO: 1105) | ID NO: |
| | | ID NO: | | 1104) | | 1106) |
| | | 1102) | | | | |
| LK-RB2- | SYGMS | TISGGGTY | GAMDY | KSSQSLLD | LVSKLDS | WQGTHFP |
| 44 | (SEQ ID | TYYPDSV | (SEQ ID | IDGKTFLN | (SEQ ID | WT (SEQ |
| | NO: 1107) | KG (SEQ | NO: 1109) | (SEQ ID | NO: 1111) | ID NO: |
| | | ID NO: | | NO: 1110) | | 1112) |
| | | 1108) | | | | |
| LK-RB2- | TYGMS | WINVYSG | RGDYDGH | RASQSISD | YVSQSIS | QNGHNFP |
| 45 | (SEQ ID | VPTYADD | YFDY | YLN (SEQ | (SEQ ID | LT (SEQ |
| | NO: 1113) | FKG (SEQ | (SEQ ID | ID NO: | NO: 1117) | ID NO: |
| | | ID NO: | NO: 1115) | 1116) | | 1118) |
| | | 1114) | | | | |
| LK-RB2- | DQIIH | YIYPRDDS | DAYWYF | KASQDVN | WASTRHT | QQHYSPP |
| 46 | (SEQ ID | IMYNEKF | DV (SEQ | TAVA | (SEQ ID | LT (SEQ |
| | NO: 1119) | KG (SEQ | ID NO: | (SEQ ID | NO: 1123) | ID NO: |
| | | ID NO: | 1121) | NO: 1122) | | 1124) |
| | | 1120) | | | | |
| LK-RB2- | SYGVH | VIWSGGS | SFHYHGS | RASQDIN | FTSRLHS | QQVNTLP |
| 47 | (SEQ ID | TDYNAAF | TFDY | NYLN | (SEQ ID | YT (SEQ |
| | NO: 1125) | IS (SEQ ID | (SEQ ID | (SEQ ID | NO: 1129) | ID NO: |
| | | NO: 1126) | NO: 1127) | NO: 1128) | | 1130) |

TABLE 7
The binding and blocking activity of anti-LILRB1 and anti-LILRB2 antibodies

| | | | Cell based RBA | Top | Cell based RBA | Top |
|---|---|---|---|---|---|---|
| | CHOK1- | CHOK1- | CHOK1- | Inhibition | CHOK1- | Inhibition |
| | hLILRB1 | hLILRB2 | hLILRB1 | CHOK1- | hLILRB2 | CHOK1- |
| | FACS | FACS | & HLA-G) | hLILRB1, | & HLA-G) | hLILRB2, |
| Antibody | EC50 (nM) | EC50 (nM) | EC50 (nM) | %) | EC50 (nM) | %) |
| LK-RB1-1 | 0.17 | Inactive | 0.32 | 78.04 | Inactive | Inactive |
| LK-RB1-2 | 0.28 | Inactive | 0.44 | 81.93 | Inactive | Inactive |
| LK-RB1-3 | 1.14 | Inactive | 2.08 | 87.04 | Inactive | Inactive |
| LK-RB1-4 | 1.00 | Inactive | 1.74 | 85.86 | Inactive | Inactive |
| LK-RB1-5 | 1.40 | Inactive | 1.97 | 23.01 | Inactive | Inactive |
| LK-RB1-6 | 0.22 | Inactive | 0.54 | 69.66 | Inactive | Inactive |
| LK-RB1-7 | 0.22 | Inactive | 0.54 | 69.66 | Inactive | Inactive |
| LK-RB1-8 | 1.14 | Inactive | 2.56 | 86.94 | Inactive | Inactive |
| LK-RB1-9 | 0.34 | Inactive | 0.78 | 88.13 | Inactive | Inactive |
| LK-RB1- | 0.70 | Inactive | 1.04 | 83.09 | Inactive | Inactive |
| 10 | | | | | | |
| LK-RB1- | 0.61 | Inactive | 1.34 | 78.93 | Inactive | Inactive |
| 11 | | | | | | |
| LK-RB1- | 0.17 | Inactive | 0.37 | 79.15 | Inactive | Inactive |
| 12 | | | | | | |
| LK-RB1- | 0.20 | Inactive | 0.42 | 81.85 | Inactive | Inactive |
| 13 | | | | | | |
| LK-RB1- | 0.47 | Inactive | 0.65 | 81.24 | Inactive | Inactive |
| 14 | | | | | | |
| LK-RB1- | 0.64 | Inactive | 0.79 | 67.26 | Inactive | Inactive |
| 15 | | | | | | |
| LK-RB1- | 0.81 | Inactive | 0.53 | 98.19 | Inactive | Inactive |
| 16 | | | | | | |
| LK-RB1- | 5.53 | 47.42 | untested | untested | Inactive | Inactive |
| 17 | | | | | | |
| LK-RB1- | 0.58 | Inactive | 0.47 | 98.71 | Inactive | Inactive |
| 18 | | | | | | |
| LK-RB1- | 0.30 | Inactive | 0.49 | 98.17 | Inactive | Inactive |
| 19 | | | | | | |
| LK-RB1- | 0.85 | Inactive | 0.52 | 97.44 | Inactive | Inactive |
| 20 | | | | | | |
| LK-RB1- | 1.88 | Inactive | 0.53 | 98.13 | Inactive | Inactive |
| 21 | | | | | | |
| LK-RB1- | 1.76 | Inactive | 1.40 | 100.20 | Inactive | Inactive |
| 22 | | | | | | |
| LK-RB1- | 0.73 | Inactive | 0.56 | 95.31 | Inactive | Inactive |
| 23 | | | | | | |
| LK-RB1- | 1.48 | 1.04 | 9.07 | 64.76 | Inactive | Inactive |
| 24 | | | | | | |
| LK-RB1- | 1.07 | Inactive | 0.62 | 98.14 | Inactive | Inactive |
| 25 | | | | | | |
| LK-RB1- | 0.51 | Inactive | 0.69 | 99.61 | Inactive | Inactive |
| 26 | | | | | | |
| LK-RB1- | 0.71 | 4.95 | 0.90 | 98.65 | Inactive | Inactive |
| 27 | | | | | | |
| LK-RB1- | 1.33 | 5.72 | untested | untested | Inactive | Inactive |
| 28 | | | | | | |
| LK-RB1- | 2.38 | Inactive | 2.93 | 93.29 | Inactive | Inactive |
| 29 | | | | | | |
| LK-RB1- | 1.06 | Inactive | 0.70 | 95.61 | Inactive | Inactive |
| 30 | | | | | | |
| LK-RB1- | 2.91 | Inactive | untested | untested | Inactive | Inactive |
| 31 | | | | | | |
| LK-RB1- | 2.44 | Inactive | untested | untested | Inactive | Inactive |
| 32 | | | | | | |
| LK-RB1- | 1.13 | Inactive | 1.38 | 92.65 | Inactive | Inactive |
| 33 | | | | | | |
| LK-RB1- | 1.56 | Inactive | 3.69 | 21.66 | Inactive | Inactive |
| 34 | | | | | | |
| LK-RB1- | 0.44 | Inactive | Inactive | Inactive | Inactive | Inactive |
| 35 | | | | | | |
| LK-RB1- | 0.46 | Inactive | Inactive | Inactive | Inactive | Inactive |
| 36 | | | | | | |
| LK-RB1- | 2.51 | Inactive | untested | untested | Inactive | Inactive |
| 37 | | | | | | |
| LK-RB1- | 2.45 | Inactive | untested | untested | Inactive | Inactive |
| 38 | | | | | | |
| LK-RB1- | 0.77 | 0.17 | 0.68 | 95.70 | Inactive | Inactive |
| 39 | | | | | | |
| LK-RB1- | Inactive | Inactive | Inactive | Inactive | Inactive | Inactive |
| 40 | | | | | | |
| LK-RB1- | Inactive | Inactive | Inactive | Inactive | Inactive | Inactive |
| 41 | | | | | | |
| LK-RB1- | 0.89 | Inactive | 4.50 | 62.20 | Inactive | Inactive |
| 42 | | | | | | |
| LK-RB1- | Inactive | Inactive | Inactive | Inactive | Inactive | Inactive |
| 43 | | | | | | |
| LK-RB1- | 2.79 | Inactive | Inactive | Inactive | Inactive | Inactive |
| 44 | | | | | | |
| LK-RB1- | 1.78 | Inactive | Inactive | Inactive | Inactive | Inactive |
| 45 | | | | | | |
| LK-RB1- | 0.97 | Inactive | Inactive | Inactive | Inactive | Inactive |
| 46 | | | | | | |
| LK-RB1- | 1.13 | Inactive | 1.59 | 99.00 | Inactive | Inactive |
| 47 | | | | | | |
| LK-RB1- | 1.06 | Inactive | 1.00 | 86.68 | Inactive | Inactive |
| 48 | | | | | | |
| LK-RB1- | 2.05 | Inactive | 2.32 | 91.43 | Inactive | Inactive |
| 49 | | | | | | |
| LK-RB1- | 2.01 | 605.10 | untested | untested | Inactive | Inactive |
| 50 | | | | | | |
| LK-RB1- | Inactive | Inactive | Inactive | Inactive | Inactive | Inactive |
| 51 | | | | | | |
| LK-RB1- | 1.80 | Inactive | 2.25 | 89.66 | Inactive | Inactive |
| 52 | | | | | | |
| LK-RB1- | 0.71 | Inactive | 2.01 | 75.72 | Inactive | Inactive |
| 53 | | | | | | |
| LK-RB1- | untested | Inactive | untested | untested | Inactive | Inactive |
| 54 | | | | | | |
| LK-RB1- | 1.68 | Inactive | 1.26 | 92.56 | Inactive | Inactive |
| 55 | | | | | | |
| LK-RB1- | 1.24 | Inactive | untested | untested | Inactive | Inactive |
| 56 | | | | | | |
| LK-RB1- | 1.78 | Inactive | 1.67 | 90.00 | Inactive | Inactive |
| 57 | | | | | | |
| LK-RB1- | 5.00 | Inactive | untested | untested | Inactive | Inactive |
| 58 | | | | | | |
| LK-RB1- | 0.65 | Inactive | 1.61 | 87.28 | Inactive | Inactive |
| 59 | | | | | | |
| LK-RB2-1 | Inactive | 0.24 | untested | untested | 0.3234 | 73.6863217 |
| LK-RB2-2 | Inactive | 0.25 | untested | untested | 0.1339 | 74.5475401 |
| LK-RB2-3 | Inactive | 0.40 | untested | untested | 0.9464 | 73.2407785 |
| LK-RB2-4 | Inactive | 1.61 | untested | untested | 1.706 | 65.23 |
| LK-RB2-5 | 1.34 | 0.27 | 2.86 | 70.76 | untested | untested |
| LK-RB2-6 | Inactive | 0.66 | untested | untested | untested | untested |
| LK-RB2-7 | Inactive | 0.25 | untested | untested | untested | untested |
| LK-RB2-8 | 4.55 | 7.23 | 1.32 | 32.68 | Inactive | Inactive |
| LK-RB2-9 | Inactive | 0.17 | untested | untested | untested | untested |
| LK-RB2- | Inactive | 0.27 | untested | untested | untested | untested |
| 10 | | | | | | |
| LK-RB2- | Inactive | 0.27 | untested | untested | untested | untested |
| 11 | | | | | | |
| LK-RB2- | 2.34 | 0.16 | 2.16 | 70.28 | untested | untested |
| 12 | | | | | | |
| LK-RB2- | Inactive | 0.23 | untested | untested | untested | untested |
| 13 | | | | | | |
| LK-RB2- | Inactive | 0.29 | untested | untested | 0.2312 | 73.7779741 |
| 14 | | | | | | |
| LK-RB2- | 31.38 | 0.16 | 4.18 | 70.78 | 0.1616 | 77.4587985 |
| 15 | | | | | | |
| LK-RB2- | 4.3 | 6.01 | 13.4 | 35.29 | Inactive | Inactive |
| 16 | | | | | | |
| LK-RB2- | Inactive | 0.16 | untested | untested | untested | untested |
| 17 | | | | | | |
| LK-RB2- | Inactive | 0.14 | untested | untested | untested | untested |
| 18 | | | | | | |
| LK-RB2- | 7.059 | 2.34 | untested | untested | 50.29 | 18.07 |
| 19 | | | | | | |
| LK-RB2- | Inactive | 0.14 | untested | untested | Inactive | Inactive |
| 20 | | | | | | |
| LK-RB2- | Inactive | 2.28 | untested | untested | 1.607 | 62.35 |
| 21 | | | | | | |
| LK-RB2- | 45.31 | 0.22 | untested | untested | 0.1122 | 71.06 |
| 22 | | | | | | |
| LK-RB2- | 65.5 | 0.26 | untested | untested | 0.1977 | 71.2 |
| 23 | | | | | | |
| LK-RB2- | 0.8477 | 0.18 | 1.7 | 93.41 | 0.09563 | 74.06 |
| 24 | | | | | | |
| LK-RB2- | 1.709 | Inactive | Inactive | Inactive | untested | untested |
| 25 | | | | | | |
| LK-RB2- | 4032 | Inactive | untested | untested | untested | untested |
| 26 | | | | | | |
| LK-RB2- | Inactive | Inactive | untested | untested | untested | untested |
| 27 | | | | | | |
| LK-RB2- | Inactive | Inactive | untested | untested | untested | untested |
| 28 | | | | | | |
| LK-RB2- | Inactive | Inactive | untested | untested | untested | untested |
| 29 | | | | | | |
| LK-RB2- | Inactive | Inactive | untested | untested | untested | untested |
| 30 | | | | | | |
| LK-RB2- | 1.99 | Inactive | Inactive | Inactive | untested | untested |
| 31 | | | | | | |
| LK-RB2- | 4.539 | Inactive | Inactive | Inactive | untested | untested |
| 32 | | | | | | |
| LK-RB2- | Inactive | 0.91 | untested | untested | 1.874 | 90.03 |
| 33 | | | | | | |
| LK-RB2- | Inactive | Inactive | untested | untested | untested | untested |
| 34 | | | | | | |
| LK-RB2- | Inactive | 0.18 | untested | untested | 0.2029 | 88.37 |
| 35 | | | | | | |
| LK-RB2- | Inactive | 0.72 | untested | untested | 0.5065 | 80.61 |
| 36 | | | | | | |
| LK-RB2- | Inactive | 0.34 | untested | untested | 0.2679 | 77.33 |
| 37 | | | | | | |
| LK-RB2- | Inactive | Inactive | untested | untested | untested | untested |
| 38 | | | | | | |
| LK-RB2- | Inactive | 36.58 | untested | untested | untested | untested |
| 39 | | | | | | |
| LK-RB2- | Inactive | 0.53 | untested | untested | 0.4445 | 79.4 |
| 40 | | | | | | |
| LK-RB2- | Inactive | 0.37 | untested | untested | 0.1573 | 80.09 |
| 41 | | | | | | |
| LK-RB2- | Inactive | Inactive | untested | untested | untested | untested |
| 42 | | | | | | |
| LK-RB2- | Inactive | Inactive | untested | untested | untested | untested |
| 43 | | | | | | |
| LK-RB2- | Inactive | Inactive | untested | untested | untested | untested |
| 44 | | | | | | |
| LK-RB2- | 1.476 | 0.30 | untested | untested | 0.06725 | 81.6 |
| 45 | | | | | | |
| LK-RB2- | Inactive | 0.35 | untested | untested | untested | untested |
| 46 | | | | | | |
| LK-RB2- | Inactive | Inactive | untested | untested | untested | untested |
| 47 | | | | | | |
| 15G8 | 0.57 | Inactive | 0.69 | 82.53 | untested | untested |
| 3C1 | 0.62 | Inactive | 0.94 | 79.59 | untested | untested |
| 12D12 | untested | Inactive | 1.26 | 89.07 | untested | untested |
| J.19.h1 | Inactive | 0.15 | Inactive | Inactive | 0.08891 | 68.734573 |
| p1E1 | Inactive | 0.64 | Inactive | Inactive | untested | untested |
| ISO | Inactive | Inactive | Inactive | Inactive | Inactive | Inactive |

### Anti-LILRB1 and Anti-LILRB2 Antibody Characterization

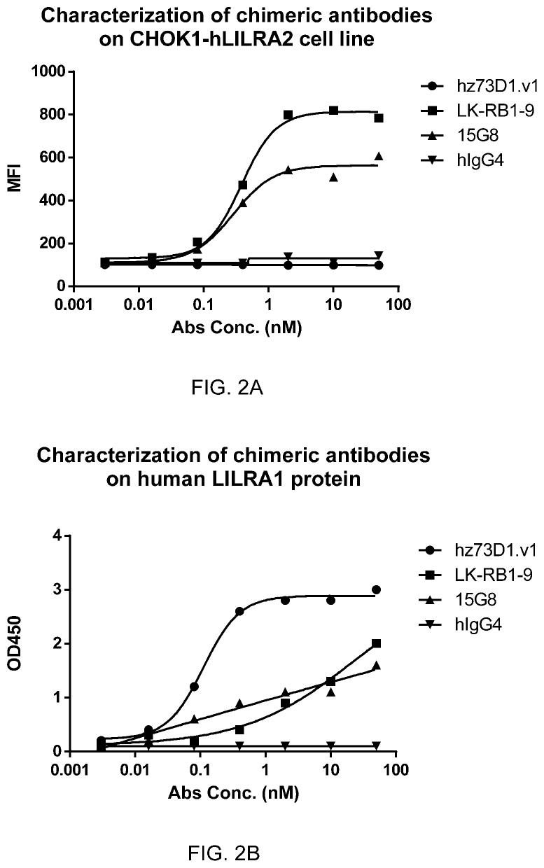

Anti-LILRB1 and anti-LILRB2 antibodies were assayed for binding specificity against closely related LILRB family member human LILRB3-5 (Sino Biological, LLC: 16014-H02H, 11978-H08H, 16742-H08H and 17221-H08H) and human LILRA family member LILRA1-6 (Sino Biological, LLC: 17220-H08H, 30010-H08H, 13549-H08H, 16058-H08H, 16059-H08H and 11978-H08H) by either FACS or ELISA analysis. As summarized in Table 8, different clones of anti-LILRB1 and anti-LILRB2 antibody had different binding selectivity against LILRB and LILRA family members. To further confirm binding affinity, selected clones were assayed in ELISA or FACS in a range of testing concentrations. As shown in FIG. 2A, LK-RB1-9 and 15G8 bind to human LILRA2 with relatively high affinity, but hz73D1.v1 had no binding to LILRA2. However, for binding to LILRA1, hz73D1.v1 had high binding affinity comparing to LK-RB1-9 and 15G8 (FIG. 2B).

TABLE 8
The binding cross-reactivity of anti-LILRB1 and anti-LILRB2 antibodies to
LILRB and LILRA family members

| | hLILR | hLILR | hLILR | hLILR | hLILR | hLILR | hLILR | hLILR | hLILR |
|---|---|---|---|---|---|---|---|---|---|
| | A1 | A2 | A3 | A4 | A5 | A6 | B3 | B4 | B5 |
| | ELISA | ELISA | ELISA | ELISA | ELISA | ELISA | ELISA | ELISA | ELISA |
| | (200 | (200 | (200 | (200 | (200 | (200 | (200 | (200 | (200 |
| | nM; | nM; | nM; | nM; | nM; | nM; | nM; | nM; | nM; |
| | OD450; | OD450, | OD450, | OD450, | OD450, | OD450, | OD450, | OD450, | OD450, |
| Antibody | +/−) | +/−) | +/−) | +/−) | +/−) | +/−) | +/−) | +/−) | +/−) |
| LK- | + | + | − | + | − | − | − | + | − |
| RB1-1 | | | | | | | | | |
| LK- | + | + | − | + | − | − | − | − | − |
| RB1-2 | | | | | | | | | |
| LK- | − | − | + | − | − | − | − | − | − |
| RB1-3 | | | | | | | | | |
| LK- | − | − | + | − | − | | − | − | − |
| RB1-4 | | | | | | | | | |
| LK- | − | + | − | − | − | + | + | − | + |
| RB1-5 | | | | | | | | | |
| LK- | − | + | + | + | − | − | − | − | − |
| RB1-6 | | | | | | | | | |
| LK- | − | + | + | + | − | − | − | − | − |
| RB1-7 | | | | | | | | | |
| LK- | − | − | − | − | − | − | − | − | − |
| RB1-8 | | | | | | | | | |
| LK- | + | − | − | − | − | − | − | − | − |
| RB1-9 | | | | | | | | | |
| LK- | + | − | + | − | − | − | − | − | − |
| RB1-10 | | | | | | | | | |
| LK- | + | − | + | − | − | − | − | − | − |
| RB1-11 | | | | | | | | | |
| LK- | − | − | − | − | − | − | − | − | − |
| RB1-12 | | | | | | | | | |
| LK- | + | + | + | − | − | − | − | − | − |
| RB1-13 | | | | | | | | | |
| LK- | + | − | + | − | − | − | − | − | − |
| RB1-14 | | | | | | | | | |
| LK- | + | − | + | − | − | − | − | − | − |
| RB1-15 | | | | | | | | | |
| LK- | − | − | − | − | − | − | − | − | − |
| RB1-16 | | | | | | | | | |
| LK- | − | − | − | − | − | − | − | − | − |
| RB1-17 | | | | | | | | | |
| LK- | − | − | − | − | − | − | − | − | − |
| RB1-24 | | | | | | | | | |
| LK- | − | − | − | − | − | − | − | − | − |
| RB1-27 | | | | | | | | | |
| LK- | − | − | − | − | − | − | − | − | − |
| RB1-28 | | | | | | | | | |
| LK- | − | − | − | − | − | − | − | − | − |
| RB1-29 | | | | | | | | | |
| LK- | − | − | − | − | − | − | − | − | − |
| RB1-30 | | | | | | | | | |
| LK- | − | + | − | − | − | − | − | − | − |
| RB1-33 | | | | | | | | | |
| LK- | − | − | − | − | − | − | + | − | + |
| RB1-35 | | | | | | | | | |
| LK- | − | − | − | − | − | − | + | − | + |
| RB1-36 | | | | | | | | | |
| LK- | + | − | + | − | − | − | + | − | + |
| RB1-39 | | | | | | | | | |
| LK- | − | − | + | − | − | − | − | − | − |
| RB1-45 | | | | | | | | | |
| LK- | − | − | − | − | − | − | − | − | − |
| RB1-46 | | | | | | | | | |
| LK- | + | + | − | − | − | − | − | − | − |
| RB1-47 | | | | | | | | | |
| LK- | + | − | − | − | − | − | − | − | − |
| RB1-48 | | | | | | | | | |
| LK- | − | − | − | − | − | − | − | − | − |
| RB1-50 | | | | | | | | | |
| LK- | + | − | − | − | − | − | − | − | − |
| RB1-55 | | | | | | | | | |
| LK- | + | + | − | − | − | − | − | − | − |
| RB1-59 | | | | | | | | | |
| LK- | − | − | − | − | − | − | − | − | − |
| RB2-1 | | | | | | | | | |
| LK- | − | − | − | − | − | − | − | − | − |
| RB2-2 | | | | | | | | | |
| LK- | − | − | − | − | − | − | − | − | − |
| RB2-3 | | | | | | | | | |
| LK- | − | − | − | − | − | − | − | − | − |
| RB2-4 | | | | | | | | | |
| LK- | − | − | − | − | − | − | − | − | − |
| RB2-5 | | | | | | | | | |
| LK- | − | − | − | − | − | − | − | − | − |
| RB2-6 | | | | | | | | | |
| LK- | − | − | − | − | − | − | − | − | − |
| RB2-7 | | | | | | | | | |
| LK- | + | − | + | + | + | + | + | + | + |
| RB2-8 | | | | | | | | | |
| LK- | − | − | − | − | − | − | − | − | − |
| RB2-9 | | | | | | | | | |
| LK- | − | + | − | − | − | − | − | − | − |
| RB2-10 | | | | | | | | | |
| LK- | − | − | − | − | − | − | − | − | − |
| RB2-11 | | | | | | | | | |
| LK- | + | − | − | − | − | − | − | + | − |
| RB2-12 | | | | | | | | | |
| LK- | − | − | − | − | − | − | − | − | − |
| RB2-13 | | | | | | | | | |
| LK- | − | − | − | − | − | − | − | − | − |
| RB2-14 | | | | | | | | | |
| LK- | + | − | − | − | − | − | − | + | − |
| RB2-15 | | | | | | | | | |
| LK- | + | − | − | + | − | + | + | − | + |
| RB2-16 | | | | | | | | | |
| LK- | − | − | − | − | − | − | − | − | − |
| RB2-17 | | | | | | | | | |
| LK- | − | − | − | − | − | − | − | − | − |
| RB2-18 | | | | | | | | | |
| LK- | − | − | − | − | − | − | − | − | − |
| RB2-19 | | | | | | | | | |
| LK- | − | − | − | − | − | − | − | − | − |
| RB2-20 | | | | | | | | | |
| LK- | − | − | − | − | − | − | − | − | − |
| RB2-21 | | | | | | | | | |
| LK- | − | − | − | − | − | − | − | − | − |
| RB2-22 | | | | | | | | | |
| LK- | − | − | − | − | − | − | − | − | − |
| RB2-23 | | | | | | | | | |
| LK- | + | − | − | − | − | − | − | − | − |
| RB2-24 | | | | | | | | | |
| LK- | − | − | − | − | − | − | − | − | − |
| RB2-25 | | | | | | | | | |
| LK- | − | − | − | − | − | − | − | − | − |
| RB2-26 | | | | | | | | | |
| LK- | − | − | + | − | − | − | − | − | − |
| RB2-27 | | | | | | | | | |
| LK- | − | − | − | − | − | − | − | − | − |
| RB2-28 | | | | | | | | | |
| LK- | + | + | − | − | − | − | − | − | − |
| RB2-29 | | | | | | | | | |
| LK- | − | − | − | − | − | − | − | − | − |
| RB2-30 | | | | | | | | | |
| LK- | − | − | − | − | − | − | − | − | − |
| RB2-31 | | | | | | | | | |
| LK- | − | − | − | − | − | − | − | − | − |
| RB2-32 | | | | | | | | | |
| LK- | − | − | − | − | − | − | − | − | − |
| RB2-33 | | | | | | | | | |
| LK- | − | − | − | − | − | − | − | − | − |
| RB2-34 | | | | | | | | | |
| LK- | − | − | − | − | − | − | − | − | − |
| RB2-35 | | | | | | | | | |
| LK- | − | − | − | − | − | − | − | − | − |
| RB2-36 | | | | | | | | | |
| LK- | − | − | − | − | − | − | − | − | − |
| RB2-37 | | | | | | | | | |
| LK- | − | − | − | − | − | − | − | − | − |
| RB2-38 | | | | | | | | | |
| LK- | − | − | − | − | − | − | − | − | − |
| RB2-39 | | | | | | | | | |
| LK- | − | − | − | − | − | − | − | − | − |
| RB2-40 | | | | | | | | | |
| LK- | + | + | − | − | − | − | − | − | − |
| RB2-41 | | | | | | | | | |
| LK- | − | − | − | − | − | − | − | − | − |
| RB2-42 | | | | | | | | | |
| LK- | − | − | − | − | − | − | − | − | − |
| RB2-43 | | | | | | | | | |
| LK- | − | − | − | − | − | − | − | − | − |
| RB2-44 | | | | | | | | | |
| LK- | − | − | − | − | − | − | − | − | − |
| RB2-45 | | | | | | | | | |
| LK- | − | − | − | − | − | − | − | − | − |
| RB2-46 | | | | | | | | | |
| LK- | − | − | − | − | − | − | − | − | − |
| RB2-47 | | | | | | | | | |
| Positive | + | + | + | + | + | + | + | + | + |
| Control | | | | | | | | | |
| ISO | − | − | − | − | − | − | − | − | − |

+: 2-fold over ISO was regarded as positive (+).

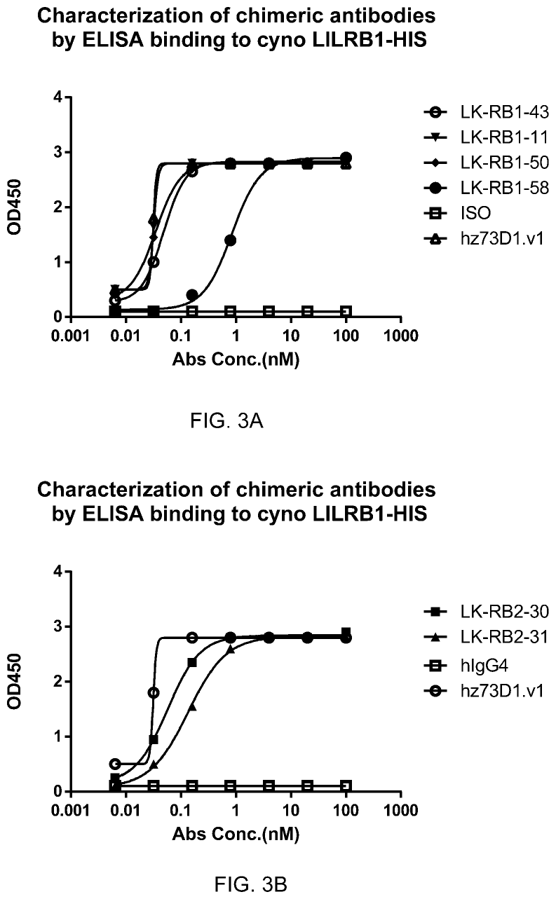

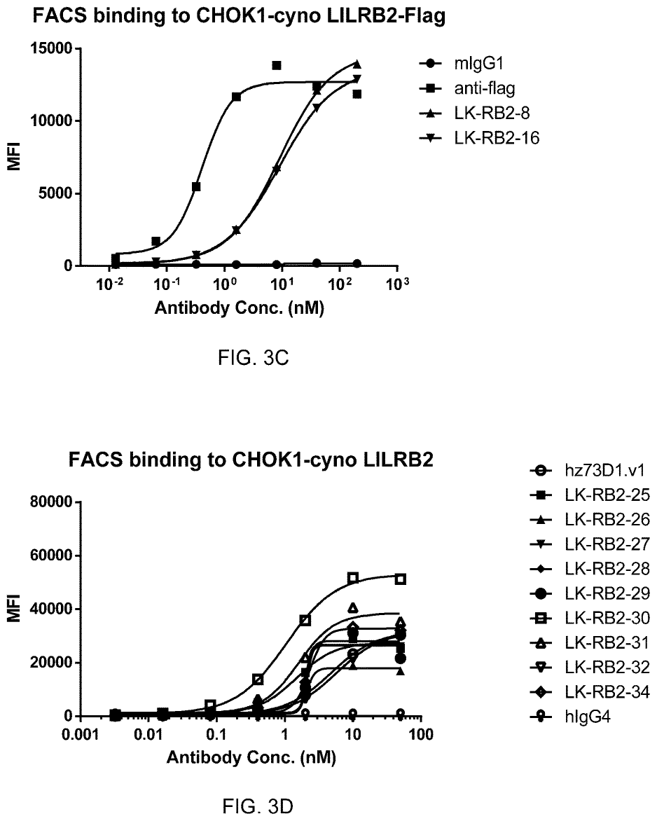

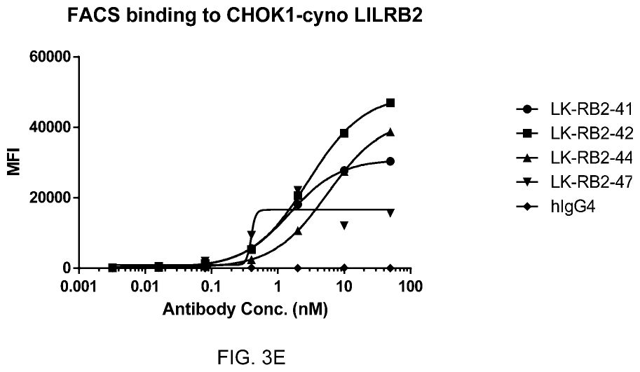

Anti-LILRB1 and anti-LILRB2 antibodies were also screened for their ability to bind rhesus macaque LILRB1 (Kactusbio, LLC: LIL-CM1B1 and LIL-CM1B3), cynomolgus LILRB1 (Kactusbio, LLC: LIL-CM1B2) and cynomolgus LILRB2 (Kactusbio, LLC: LIL-CM1B5) by ELISA and flow cytometry. As shown in Table 9 below, some anti-LILRB1 and anti-LILRB2 antibodies bind to rhesus macaque LILRB1 and/or cynomolgus LILRB2. The binding against cynomolgus LILRB1 was further confirmed in a dose response manner (FIGS. 3A-3B). hz73D1.v1, LK-RB1-43, LK-RB1-11, LK-RB1-50, LK-RB1-58, LK-RB2-30 and LK-RB2-31 all dose dependently bind to cynomolgus LILRB1 protein by ELISA. The binding against cynomolgus LILRB2 was further confirmed in a dose response manner (FIGS. 3C-3E). hz73D1.v1, LK-RB2-8, LK-RB2-16, LK-RB2-25, LK-RB2-26, LK-RB2-27, LK-RB2-28, LK-RB2-29, LK-RB2-30, LK-RB2-31, LK-RB2-32, LK-RB2-34, LK-RB2-41, LK-RB2-42, LK-RB2-44 and LK-RB2-47 all dose dependently bind to cynomolgus LILRB2 overexpressing CHOK1 cell line (Celetrix, LLC: Accession: XM_015441717) by flow cytometry.

TABLE 9
The binding activity of anti-LILRB1 and anti-LILRB2 antibodies to rhesus
macaque/cynomolgus LILRB1 and LILRB2

| | | | | Cell based | | |
|---|---|---|---|---|---|---|
| | | | Cell based | blocking assay | | |
| | | | blocking | [CHOK1- | | |
| | | CHOK1-Rhesus | assay | Rhesus | CHOK1- | |
| | Rhesus | LILRB1 | [CHOK1- | LILRB1 | Rhesus | |
| | LILRB1 | (NP_00103 | Rhesus | (NP_00103 | LILRB1 | CHOK1- |
| | (NP_00103 | 5762.2) | LILRB1 | 5762.2)] | (F7H3G7) | Cyno |
| | 5762.2) | FACS | (NP_00103 | Top | FACS | LILRB2 |
| | ELISA | binding | 5762.2)] | Inhibition | binding | FACS |
| Antibody | EC50 (nM) | EC50 (nM) | EC50 (nM) | (%) | EC50 (nM) | EC50 (nM) |
| LK-RB1-5 | 0.05 | 1.08 | Inactive | Inactive | 1.51 | 1.16 |
| LK-RB1-11 | 0.08 | Inactive | Inactive | Inactive | 0.78 | Inactive |
| LK-RB1-34 | Inactive | 8.06 | Inactive | Inactive | Inactive | Inactive |
| LK-RB1-35 | Inactive | 0.64 | Inactive | Inactive | Inactive | Inactive |
| LK-RB1-36 | Inactive | 0.54 | Inactive | Inactive | Inactive | Inactive |
| LK-RB1-37 | Inactive | 37.67 | Inactive | Inactive | Inactive | Inactive |
| LK-RB1-38 | Inactive | 4.97 | Inactive | Inactive | Inactive | Inactive |
| LK-RB1-39 | Inactive | 0.36 | 0.16 | 73.13 | Inactive | 0.36 |
| LK-RB1-42 | Inactive | 59.84 | Inactive | Inactive | Inactive | Inactive |
| LK-RB1-43 | Inactive | 1.62 | Inactive | Inactive | Inactive | Inactive |
| LK-RB1-44 | Inactive | 769.00 | Inactive | Inactive | Inactive | Inactive |
| LK-RB1-45 | Inactive | 96.14 | Inactive | Inactive | Inactive | Inactive |
| LK-RB1-46 | Inactive | 2.71 | Inactive | Inactive | Inactive | Inactive |
| LK-RB1-50 | Inactive | 11.15 | 5.68 | 57.84 | 19886.00 | Inactive |
| LK-RB1-51 | Inactive | 21.41 | 0.87 | 64.88 | 26617.00 | Inactive |
| LK-RB1-54 | Inactive | Inactive | 1.20 | 54.01 | 24183.00 | Inactive |
| LK-RB1-56 | Inactive | 29.25 | 2.42 | 15.87 | Inactive | Inactive |
| LK-RB1-58 | Inactive | 158.30 | 1.42 | 45.40 | 17917.00 | Inactive |
| LK-RB1-59 | Inactive | 40.23 | Inactive | Inactive | Inactive | Inactive |
| LK-RB2-8 | untested | 15.06 | untested | untested | untested | 19.79 |
| LK-RB2-16 | untested | 18.47 | untested | untested | untested | 5.45 |
| LK-RB2-25 | untested | untested | untested | untested | untested | 1.48 |
| LK-RB2-26 | untested | untested | untested | untested | untested | 2.11 |
| LK-RB2-27 | untested | untested | untested | untested | untested | 2.14 |
| LK-RB2-28 | untested | untested | untested | untested | untested | 2.03 |
| LK-RB2-29 | untested | untested | untested | untested | untested | 2.09 |
| LK-RB2-30 | untested | untested | untested | untested | untested | 1.04 |
| LK-RB2-31 | untested | untested | untested | untested | untested | 1.59 |
| LK-RB2-32 | untested | untested | untested | untested | untested | 5.72 |
| LK-RB2-34 | untested | untested | untested | untested | untested | 2.30 |
| LK-RB2-39 | untested | untested | untested | untested | untested | Inactive |
| LK-RB2-40 | untested | untested | untested | untested | untested | Inactive |
| LK-RB2-41 | untested | untested | untested | untested | untested | 1.50 |
| LK-RB2-42 | untested | untested | untested | untested | untested | 2.86 |
| LK-RB2-44 | untested | untested | untested | untested | untested | 5.70 |
| LK-RB2-47 | untested | untested | untested | untested | untested | 0.40 |
| hz73D1.v1 | Inactive | Inactive | Inactive | Inactive | 1.25 | Inactive |
| 15G8 | Inactive | 24.14 | 4.56 | 88.07 | 760.30 | 2.15 |
| ISO | Inactive | Inactive | Inactive | Inactive | Inactive | Inactive |

Binding affinity of anti-LILRB1 and anti-LILRB2 antibodies against human LILRB1, LILRB2, LILRA1, LILRA2 and cynomolgus LILRB2 was ranked by measuring dissociation rate constant (Kdis) using the Octet system. As shown in Table 10 below, the selected anti-LILRB1 and anti-LILRB2 antibodies all had good binding affinity against human LILRB1 or human LILRB2. Some of the clones showed cross reactivity to other family member. For example, LK-RB1-9 had similar binding dissociation rate to human LILRB1 and human LILRA1, but bind relatively weaker to human LILRA2. LK-RB2-24 had similar binding dissociation rate to human LILRB1 and human LILRB2. LK-RB2-32 had similar binding dissociation rate to human LILRB1, human LILRB2 and cynomolgus LILRB2.

TABLE 10
The binding dissociation rate constant (kdis) for anti-LILRB1
and LILRB2 antibodies to human LILRB1, human LILRB2,
human LILRA1, human LILRA2 and cynomolgus LILRB2

| | | | | | Off Rate |
|---|---|---|---|---|---|
| | Off Rate | Off Rate | Off Rate | Off Rate | ranking |
| | ranking | ranking | ranking | ranking | [Cyno |
| | [hLILRB1, | [hLILRB2, | [hLILRA1, | [hLILRA2, | LILRB2, |
| Antibody | kdis(1/s)] | kdis(1/s)] | kdis(1/s)] | kdis(1/s)] | kdis(1/s)] |
| LK-RB1-2 | 1.18E−04 | / | / | / | / |
| LK-RB1-3 | 5.24E−05 | / | / | / | / |
| LK-RB1-4 | 2.79E−05 | / | / | / | / |
| LK-RB1-6 | 5.90E−04 | / | / | / | / |
| LK-RB1-7 | 5.90E−04 | / | / | / | / |
| LK-RB1-8 | 2.18E−04 | / | / | / | / |
| LK-RB1-9 | 1.62E−04 | / | 1.55E−04 | 8.85E−04 | / |
| LK-RB1-12 | 1.26E−03 | / | / | / | / |
| LK-RB1-14 | 1.31E−03 | / | / | / | / |
| LK-RB1-15 | 1.03E−04 | / | / | / | / |
| LK-RB1-16 | 5.92E−05 | / | / | / | / |
| LK-RB1-17 | 3.12E−04 | / | / | / | / |
| LK-RB1-18 | 8.15E−05 | / | / | / | / |
| LK-RB1-19 | 4.78E−05 | / | / | / | / |
| LK-RB1-20 | 1.41E−04 | / | / | / | / |
| LK-RB1-21 | 4.02E−05 | / | / | / | / |
| LK-RB1-22 | 4.16E−04 | / | / | / | / |
| LK-RB1-23 | 4.77E−04 | / | / | / | / |
| LK-RB1-24 | 9.87E−04 | / | / | / | / |
| LK-RB1-25 | 9.03E−05 | / | / | / | / |
| LK-RB1-26 | 1.55E−04 | / | / | / | / |
| LK-RB1-27 | 1.12E−04 | / | / | / | / |
| LK-RB1-28 | 6.28E−05 | / | / | / | / |
| LK-RB1-29 | 8.11E−05 | / | / | / | / |
| LK-RB1-30 | 6.05E−05 | / | / | / | / |
| LK-RB1-31 | 6.77E−05 | / | / | / | / |
| LK-RB1-32 | 3.66E−05 | / | / | / | / |
| LK-RB1-33 | 1.55E−04 | / | / | / | / |
| LK-RB1-34 | 2.22E−04 | / | / | / | / |
| LK-RB1-35 | 1.16E−03 | / | / | / | / |
| LK-RB1-36 | 1.21E−03 | / | / | / | / |
| LK-RB1-37 | 3.91E−05 | / | / | / | / |
| LK-RB1-38 | 5.32E−04 | / | / | / | / |
| LK-RB1-39 | 4.64E−05 | 1.53E−03 | / | / | / |
| LK-RB1-42 | 1.39E−04 | / | / | | / |
| LK-RB1-43 | 3.64E−04 | / | / | / | / |
| LK-RB1-44 | 5.59E−04 | | / | / | / |
| LK-RB1-45 | 6.11E−04 | / | / | / | / |
| LK-RB1-46 | 7.39E−04 | / | / | / | / |
| LK-RB1-47 | 8.28E−04 | / | / | / | / |
| LK-RB1-48 | 8.47E−06 | / | / | / | / |
| LK-RB1-55 | <1.0E−07 | / | / | / | / |
| LK-RB1-57 | <1.0E−07 | / | / | / | / |
| hz73D1.v1 | 5.18E−05 | / | 7.52E−06 | / | / |
| LK-RB2-1 | / | 3.41E−03 | / | / | / |
| LK-RB2-2 | / | 7.57E−04 | / | / | / |
| LK-RB2-3 | / | 3.91E−05 | / | / | / |
| LK-RB2-4 | / | 1.57E−04 | / | / | / |
| LK-RB2-6 | / | 1.67E−03 | / | / | / |
| LK-RB2-7 | / | 1.40E−03 | / | / | / |
| LK-RB2-8 | 4.85E−05 | 1.00E−07 | / | / | / |
| LK-RB2-9 | / | 1.35E−03 | / | / | / |
| LK-RB2-12 | / | 6.62E−04 | / | / | / |
| LK-RB2-13 | / | 3.04E−03 | / | / | / |
| LK-RB2-14 | / | 3.02E−04 | / | / | / |
| LK-RB2-15 | / | 6.22E−05 | / | / | / |
| LK-RB2-16 | 2.38E−04 | 5.50E−05 | / | / | / |
| LK-RB2-17 | / | 9.26E−04 | / | / | / |
| LK-RB2-18 | / | 1.74E−04 | / | / | / |
| LK-RB2-19 | / | 2.00E−03 | / | / | / |
| LK-RB2-20 | / | 8.95E−04 | / | / | / |
| LK-RB2-21 | / | 9.66E−04 | / | / | / |
| LK-RB2-22 | / | 1.26E−03 | / | / | / |
| LK-RB2-23 | / | 8.76E−04 | / | / | / |
| LK-RB2-24 | 2.05E−04 | 2.44E−04 | / | / | / |
| LK-RB2-25 | 6.09E−04 | / | / | / | 4.24E−04 |
| LK-RB2-26 | / | / | / | / | 1.84E−03 |
| LK-RB2-27 | / | / | / | / | 1.09E−03 |
| LK-RB2-28 | / | / | / | / | 1.14E−03 |
| LK-RB2-29 | / | / | / | / | 2.15E−04 |
| LK-RB2-30 | / | / | / | / | 2.01E−05 |
| LK-RB2-31 | 1.67E−03 | / | / | / | 2.91E−04 |
| LK-RB2-32 | 1.86E−03 | 2.91E−03 | / | / | 1.23E−03 |
| LK-RB2-33 | / | 1.39E−03 | / | / | / |
| LK-RB2-34 | / | / | / | / | 6.73E−04 |
| LK-RB2-35 | / | 1.20E−03 | / | / | / |
| LK-RB2-41 | / | 6.33E−06 | / | / | 1.19E−03 |
| LK-RB2-42 | / | / | / | / | 1.47E−04 |
| LK-RB2-44 | / | / | / | / | 3.46E−04 |
| LK-RB2-45 | / | 8.84E−05 | / | / | / |
| LK-RB2-47 | / | / | / | / | 3.03E−05 |
| 15G8 | 3.89E−03 | / | 2.78E−04 | 1.17E−03 | / |
| 12D12 | 3.38E−04 | / | / | / | / |
| ISO | Inactive | Inactive | Inactive | Inactive | Inactive |

### Epitope Binning

Binding epitope of a subset of anti-LILRB1 and anti-LILRB2 antibodies was analyzed by epitope binning ELISA. Briefly, a panel of testing antibodies were coated onto ELISA plate. 50 ul/well Biotinylated LILRB1 or LILRB2 and 50 ul/well of competing antibodies were added to the coated plate and incubated at 37° C. for 1 hour. After Streptavidin-HRP (1:5000) incubation and TMB development, the plates were read at 450 nm using Molecular device spectra max plus384. It was considered that the two antibodies shared similar binding epitope if the competing antibody could inhibit biotinylated-antigen binding to the coating antibody in ELISA. Epitope binning results are summarized in Table 11 and Table 12 below. Antibody LK-RB1-31, hz73D1.v1, and LK-RB1-12 fell in close but not entirely overlapping epitope bin. Antibody LK-RB1-9, LK-RB1-33, and 15G8 fell in close but not entirely overlapping epitope bin. Antibody LK-RB2-14, LK-RB2-15 and LK-RB2-24 may share similar binding epitope as they could block each other's binding to biotinylated-LILRB2 protein. Antibody LK-RB2-2 and hz73D1.v1 may share similar binding epitope, which may partially overlap with the binding epitope of J19.h1.

TABLE 11
Epitope binning of anti-LILRB1 antibodies

| | Competing (20 μg/ml) | | | | | | | | |
|---|---|---|---|---|---|---|---|---|---|
| | | LK- | LK- | hz73D | LK- | LK- | LK- | LK- | 15G |
| Inhibition % | | RB1-16 | RB1-31 | 1.v1 | RB1-12 | RB1-47 | RB1-9 | RB1-33 | 8 |
| Coating | LK-RB1-16 | 42.91% | 3.39% | 1.48% | 12.53% | 2.23% | 0.98% | −0.04% | 0.37% |
| 1 ug/ml, | LK-RB1-31 | 75.66% | 64.43% | 54.07% | 87.38% | 5.86% | 10.46% | 1.14% | −1.73% |
| 100 ul/ well | hz73D1.v1 | 61.20% | 41.58% | 31.38% | 82.77% | 46.88% | 57.35% | 23.48% | −3.83% |
| | LK-RB1-12 | 88.02% | 82.98% | 77.34% | 90.28% | 83.21% | 84.46% | 75.31% | −9.78% |
| | LK-RB1-47 | 1.24% | 0.59% | 74.81% | 82.08% | 89.77% | 31.78% | 21.71% | 5.80% |
| | LK-RB1-9 | 21.94% | 10.23% | 88.10% | 92.13% | 89.08% | 73.87% | 67.37% | 48.56% |
| | LK-RB1-33 | 3.49% | 4.35% | 88.65% | 68.80% | 96.48% | 94.85% | 96.04% | 72.19% |
| | 15G8 | −0.72% | −0.67% | −0.56% | 3.58% | 13.53% | 92.12% | 90.49% | 72.99% |

TABLE 12
Epitope binning of anti-LILRB2 antibodies

| | competitive Ab(20 μg/ml) | | | | | | | |
|---|---|---|---|---|---|---|---|---|
| | | LK-RB2- | LK-RB2- | LK- | LK- | LK- | | |
| Inhibition % | | 15 | 24 | RB2-14 | RB2-3 | RB2-2 | hz73D1.v1 | J19.h1 |
| Coating | LK-RB2-15 | 94.27% | 96.79% | 85.38% | 22.13% | 47.12% | 15.05% | 8.00% |
| 1 ug/ml, | LK-RB2-24 | 95.24% | 95.73% | 91.07% | 25.85% | 48.36% | 22.08% | −4.04% |
| 100 ul/well | LK-RB2-14 | 91.07% | 94.30% | 90.56% | 29.78% | 97.65% | 92.66% | 77.39% |
| | LK-RB2-3 | 5.00% | 32.24% | 24.91% | 96.67% | 96.86% | 33.74% | 9.71% |
| | LK-RB2-2 | 13.62% | 17.71% | 89.44% | 93.90% | 97.77% | 92.78% | 69.61% |
| | hz73D1.v1 | 6.43% | 22.39% | 91.76% | 65.29% | 97.75% | 96.79% | 74.78% |
| | J19.h1 | 17.46% | 44.46% | 96.13% | 93.91% | 97.78% | 95.92% | 92.55% |

### 7.2 Example 2: Humanization of Anti-LILRB1 and LILRB2 Antibodies

### Humanization Design

LK-RB1-9, LK-RB1-16, LK-RB2-2 and LK-RB2-24 were humanized by CDR grafting plus back mutation. The structure of parental antibody was modelled by MOE homology modelling program. Humanized antibodies were designed using CDR grafting. Briefly, the CDRs of parental antibody were grafted into the human acceptors to obtain humanized light chains and humanized heavy chains for each parental antibody. 4 heavy chains (VH1, VH2, VH3 and VH4) and 4 light chains (VL1, VL2, VL3 and VL4) were paired with each other for affinity ranking experiment. The PTM risk of all sequences was analyzed, and appropriate PTM removal mutations were designed of heavy chain and light chain. The sequences of humanized antibodies are shown in Tables 13-15.

Exemplary signal peptides used for antibody expression include without limitation to: MGWSCIILFLVATATGVHS (SEQ ID NO: 1229).

TABLE 13
Heavy chain and Light chain sequences of humanized antibodies

| Antibody | Heavy chain | Light chain |
|---|---|---|
| LK-RB1-9.h1 | QVQLVQSGSELKKPGASVKVSCKAS | DIQMTQSPSSLSASVGDRVTITCETS |
| | GYTFTTYGMSWVRQAPGQGLEWM | ENIYGALNWYQQKPGKAPKLLIYGA |
| | GWINTYSGVPTYADDFKGRFVFSLD | TNLAEGVPSRFSGSGSGTDFTFTISSL |
| | TSVSTAYLQISSLKAEDTAVYYCAR | QPEDIATYYCQNVLNTPWTFGGGTK |
| | RRRTYHGYLSFDYWGQGTTVTVSS | LEIKRTVAAPSVFIFPPSDEQLKSGTA |
| | ASTKGPSVFPLAPCSRSTSESTAALG | SVVCLLNNFYPREAKVQWKVDNAL |
| | CLVKDYFPEPVTVSWNSGALTSGVH | QSGNSQESVTEQDSKDSTYSLSSTLT |
| | TFPAVLQSSGLYSLSSVVTVPSSSLG | LSKADYEKHKVYACEVTHQGLSSPV |
| | TKTYTCNVDHKPSNTKVDKRVESK | TKSFNRGEC (SEQ ID NO: 1132) |
| | YGPPCPSCPAPEFLGGPSVFLFPPKPK | |
| | DTLMISRTPEVTCVVVDVSQEDPEV | |
| | QFNWYVDGVEVHNAKTKPREEQFN | |
| | STYRVVSVLTVLHQDWLNGKEYKC | |
| | KVSNKGLPSSIEKTISKAKGQPREPQ | |
| | VYTLPPSQEEMTKNQVSLTCLVKGF | |
| | YPSDIAVEWESNGQPENNYKTTPPV | |
| | LDSDGSFFLYSRLTVDKSRWQEGNV | |
| | FSCSVMHEALHNHYTQKSLSLSLGK | |
| | (SEQ ID NO: 1131) | |
| LK-RB1-9.h2 | QVQLVQSGSELKKPGASVKVSCTAS | DIQMTQSPSSLSASVGDRVTITCETS |
| | GYTFTTYGMSWVRQAPGQGLKWM | ENIYGALNWYQQKPGKAPKLLIYGA |
| | GWINTYSGVPTYADDFKGRFVFSLD | TNLAEGVPSRFSGSGSGTDYTFTISSL |
| | TSVSTAYLQISSLKAEDTAVYFCARR | QPEDIATYYCQNVLNTPWTFGGGTK |
| | RRTYHGYLSFDYWGQGTTVTVSSAS | LEIKRTVAAPSVFIFPPSDEQLKSGTA |
| | TKGPSVFPLAPCSRSTSESTAALGCL | SVVCLLNNFYPREAKVQWKVDNAL |
| | VKDYFPEPVTVSWNSGALTSGVHTF | QSGNSQESVTEQDSKDSTYSLSSTLT |
| | PAVLQSSGLYSLSSVVTVPSSSLGTK | LSKADYEKHKVYACEVTHQGLSSPV |
| | TYTCNVDHKPSNTKVDKRVESKYG | TKSFNRGEC (SEQ ID NO: 1134) |
| | PPCPSCPAPEFLGGPSVFLFPPKPKDT | |
| | LMISRTPEVTCVVVDVSQEDPEVQF | |
| | NWYVDGVEVHNAKTKPREEQFNST | |
| | YRVVSVLTVLHQDWLNGKEYKCKV | |
| | SNKGLPSSIEKTISKAKGQPREPQVY | |
| | TLPPSQEEMTKNQVSLTCLVKGFYP | |
| | SDIAVEWESNGQPENNYKTTPPVLD | |
| | SDGSFFLYSRLTVDKSRWQEGNVFS | |
| | CSVMHEALHNHYTQKSLSLSLGK | |
| | (SEQ ID NO: 1133) | |
| LK-RB1-9.h3 | QVQLVQSGSELKKPGASVKVSCTAS | DIQMTQSPSSLSASVGDRVTITCETS |
| | GYTFTTYGMSWVKQAPGQGLKWM | ENIYGALNWYQQKPGKAPKLLIYGA |
| | GWINTYSGVPTYADDFKGRFVFSLD | TNLAEGVPSRFSGSGSGTDYTFTISSL |
| | TSVSTAYLQISSLKAEDTAVYFCARR | QPEDIATYYCQNVLNTPWTFGGGTK |
| | RRTYHGYLSFDYWGQGTTVTVSSAS | LEIKRTVAAPSVFIFPPSDEQLKSGTA |
| | TKGPSVFPLAPCSRSTSESTAALGCL | SVVCLLNNFYPREAKVQWKVDNAL |
| | VKDYFPEPVTVSWNSGALTSGVHTF | QSGNSQESVTEQDSKDSTYSLSSTLT |
| | PAVLQSSGLYSLSSVVTVPSSSLGTK | LSKADYEKHKVYACEVTHQGLSSPV |
| | TYTCNVDHKPSNTKVDKRVESKYG | TKSFNRGEC (SEQ ID NO: 1136) |
| | PPCPSCPAPEFLGGPSVFLFPPKPKDT | |
| | LMISRTPEVTCVVVDVSQEDPEVQF | |
| | NWYVDGVEVHNAKTKPREEQFNST | |
| | YRVVSVLTVLHQDWLNGKEYKCKV | |
| | SNKGLPSSIEKTISKAKGQPREPQVY | |
| | TLPPSQEEMTKNQVSLTCLVKGFYP | |
| | SDIAVEWESNGQPENNYKTTPPVLD | |
| | SDGSFFLYSRLTVDKSRWQEGNVFS | |
| | CSVMHEALHNHYTQKSLSLSLGK | |
| | (SEQ ID NO: 1135) | |
| LK-RB1- | QVQLQESGPGLVKPSETLSLTCTVSG | DIQMTQSPSTLSASVGDRVTITCHAS |
| 16.h1 | FSLISNAISWIRQPPGKGLEWLGVIW | QSVDFWLNWYQQKPGKAPKLLIYK |
| | GDGNTIYHSALRSRVTISKDTSKNQF | ASNLHTGVPSRFSGSGSGTEFTLTISS |
| | SLKLSSVTAADTAVYYCAKANWDG | LQPDDFATYYCQQGQSYPLTFGQGT |
| | YYFDYWGQGTTVTVSSASTKGPSVF | KLEIKRTVAAPSVFIFPPSDEQLKSGT |
| | PLAPCSRSTSESTAALGCLVKDYFPE | ASVVCLLNNFYPREAKVQWKVDNA |
| | PVTVSWNSGALTSGVHTFPAVLQSS | LQSGNSQESVTEQDSKDSTYSLSSTL |
| | GLYSLSSVVTVPSSSLGTKTYTCNVD | TLSKADYEKHKVYACEVTHQGLSSP |
| | HKPSNTKVDKRVESKYGPPCPSCPA | VTKSFNRGEC (SEQ ID NO: 1138) |
| | PEFLGGPSVFLFPPKPKDTLMISRTPE | |
| | VTCVVVDVSQEDPEVQFNWYVDGV | |
| | EVHNAKTKPREEQFNSTYRVVSVLT | |
| | VLHQDWLNGKEYKCKVSNKGLPSSI | |
| | EKTISKAKGQPREPQVYTLPPSQEEM | |
| | TKNQVSLTCLVKGFYPSDIAVEWES | |
| | NGQPENNYKTTPPVLDSDGSFFLYS | |
| | RLTVDKSRWQEGNVFSCSVMHEAL | |
| | HNHYTQKSLSLSLGK (SEQ ID NO: | |
| | 1137) | |
| LK-RB1- | QVQLQESGPGLVKPSETLSLTCTVSG | DIQMTQSPSTLSASVGDRVTITCHAS |
| 16.h2 | FSLISNAISWIRQPPGKGLEWLGVIW | QSVDFWLNWYQQKPGKAPKLLIYK |
| | GDGNTIYHSALRSRLTISKDTSKNQV | ASNLHTGVPSRFSGSGSGTEFTLTISS |
| | FLKLSSVTAADTAVYYCAKANWDG | LQPDDFATYYCQQGQSYPLTFGQGT |
| | YYFDYWGQGTTVTVSSASTKGPSVF | KLEIKRTVAAPSVFIFPPSDEQLKSGT |
| | PLAPCSRSTSESTAALGCLVKDYFPE | ASVVCLLNNFYPREAKVQWKVDNA |
| | PVTVSWNSGALTSGVHTFPAVLQSS | LQSGNSQESVTEQDSKDSTYSLSSTL |
| | GLYSLSSVVTVPSSSLGTKTYTCNVD | TLSKADYEKHKVYACEVTHQGLSSP |
| | HKPSNTKVDKRVESKYGPPCPSCPA | VTKSFNRGEC (SEQ ID NO: 1140) |
| | PEFLGGPSVFLFPPKPKDTLMISRTPE | |
| | VTCVVVDVSQEDPEVQFNWYVDGV | |
| | EVHNAKTKPREEQFNSTYRVVSVLT | |
| | VLHQDWLNGKEYKCKVSNKGLPSSI | |
| | EKTISKAKGQPREPQVYTLPPSQEEM | |
| | TKNQVSLTCLVKGFYPSDIAVEWES | |
| | NGQPENNYKTTPPVLDSDGSFFLYS | |
| | RLTVDKSRWQEGNVFSCSVMHEAL | |
| | HNHYTQKSLSLSLGK (SEQ ID NO: | |
| | 1139) | |
| LK-RB1- | QVQLQESGPGLVKPSETLSLTCTVSG | DIQMTQSPSTLSASVGDRVTITCHAS |
| 16.h3 | FSLISNAISWIRQPPGKGLEWLGVIW | QSVDFWLNWYQQKPGKAPKLLISK |
| | GDGNTIYHSALRSRLTISKDTSKNQV | ASNLHTGVPSRFSGSGSGTEFTLTISS |
| | FLKLSSVTAADTAVYYCAKANWDG | LQPDDFATYYCQQGQSYPLTFGQGT |
| | YYFDYWGQGTTVTVSSASTKGPSVF | KLEIKRTVAAPSVFIFPPSDEQLKSGT |
| | PLAPCSRSTSESTAALGCLVKDYFPE | ASVVCLLNNFYPREAKVQWKVDNA |
| | PVTVSWNSGALTSGVHTFPAVLQSS | LQSGNSQESVTEQDSKDSTYSLSSTL |
| | GLYSLSSVVTVPSSSLGTKTYTCNVD | TLSKADYEKHKVYACEVTHQGLSSP |
| | HKPSNTKVDKRVESKYGPPCPSCPA | VTKSFNRGEC (SEQ ID NO: 1142) |
| | PEFLGGPSVFLFPPKPKDTLMISRTPE | |
| | VTCVVVDVSQEDPEVQFNWYVDGV | |
| | EVHNAKTKPREEQFNSTYRVVSVLT | |
| | VLHQDWLNGKEYKCKVSNKGLPSSI | |
| | EKTISKAKGQPREPQVYTLPPSQEEM | |
| | TKNQVSLTCLVKGFYPSDIAVEWES | |
| | NGQPENNYKTTPPVLDSDGSFFLYS | |
| | RLTVDKSRWQEGNVFSCSVMHEAL | |
| | HNHYTQKSLSLSLGK (SEQ ID NO: | |
| | 1141) | |
| LK-RB2-2.h1 | EVQLVESGGGLVKPGGSLRLSCAAS | DIQMTQSPSSVSASVGDRVTITCRAS |
| | GFTFSSYAMSWVRQAPGKGLEWVS | ENIYSNLAWYQQKPGKAPKLLIYAA |
| | YISSGGDYFHYADTVKGRFTISRDN | TNLAEGVPSRFSGSGSGTDYTLTISS |
| | AKNSLYLQMNSLRAEDTAVYYCAR | LQPEDFATYYCQQFWDNPLTFGQGT |
| | EGAHYDSSYLAWFAHWGQGTLVTV | KLEIKRTVAAPSVFIFPPSDEQLKSGT |
| | SSASTKGPSVFPLAPCSRSTSESTAAL | ASVVCLLNNFYPREAKVQWKVDNA |
| | GCLVKDYFPEPVTVSWNSGALTSGV | LQSGNSQESVTEQDSKDSTYSLSSTL |
| | HTFPAVLQSSGLYSLSSVVTVPSSSL | TLSKADYEKHKVYACEVTHQGLSSP |
| | GTKTYTCNVDHKPSNTKVDKRVES | VTKSFNRGEC (SEQ ID NO: 1144) |
| | KYGPPCPSCPAPEFLGGPSVFLFPPKP | |
| | KDTLMISRTPEVTCVVVDVSQEDPE | |
| | VQFNWYVDGVEVHNAKTKPREEQF | |
| | NSTYRVVSVLTVLHQDWLNGKEYK | |
| | CKVSNKGLPSSIEKTISKAKGQPREP | |
| | QVYTLPPSQEEMTKNQVSLTCLVKG | |
| | FYPSDIAVEWESNGQPENNYKTTPP | |
| | VLDSDGSFFLYSRLTVDKSRWQEGN | |
| | VFSCSVMHEALHNHYTQKSLSLSLG | |
| | K (SEQ ID NO: 1143) | |
| LK-RB2-2.h2 | EVQLVESGGGLVKPGGSLRLSCAAS | DIQMTQSPSSVSASVGDRVTITCRAS |
| | GFTFSSYAMSWVRQAPGKGLEWVS | ENIYSNLAWYQQKPGKAPKLLVYA |
| | YISSGGDYFHYADTVKGRFTISRDN | ATNLAEGVPSRFSGSGSGTDYTLTIS |
| | AKNSLYLQMNSLRAEDTAVYYCAR | SLQPEDFATYYCQQFWDNPLTFGQG |
| | EGAHYDSSYLAWFAHWGQGTLVTV | TKLEIKRTVAAPSVFIFPPSDEQLKSG |
| | SSASTKGPSVFPLAPCSRSTSESTAAL | TASVVCLLNNFYPREAKVQWKVDN |
| | GCLVKDYFPEPVTVSWNSGALTSGV | ALQSGNSQESVTEQDSKDSTYSLSST |
| | HTFPAVLQSSGLYSLSSVVTVPSSSL | LTLSKADYEKHKVYACEVTHQGLSS |
| | GTKTYTCNVDHKPSNTKVDKRVES | PVTKSFNRGEC (SEQ ID NO: 1146) |
| | KYGPPCPSCPAPEFLGGPSVFLFPPKP | |
| | KDTLMISRTPEVTCVVVDVSQEDPE | |
| | VQFNWYVDGVEVHNAKTKPREEQF | |
| | NSTYRVVSVLTVLHQDWLNGKEYK | |
| | CKVSNKGLPSSIEKTISKAKGQPREP | |
| | QVYTLPPSQEEMTKNQVSLTCLVKG | |
| | FYPSDIAVEWESNGQPENNYKTTPP | |
| | VLDSDGSFFLYSRLTVDKSRWQEGN | |
| | VFSCSVMHEALHNHYTQKSLSLSLG | |
| | K (SEQ ID NO: 1145) | |
| LK-RB2-2.h3 | EVQLVESGGGLVKPGGSLRLSCAAS | DIQMTQSPSSVSASVGDRVTITCRAS |
| | GFTFSSYAMSWVRQAPGKGLEWVA | ENIYSNLAWYQQKPGKAPKLLIYAA |
| | YISSGGDYFHYADTVKGRFTISRDN | TNLAEGVPSRFSGSGSGTDYTLTISS |
| | AKNSLYLQMNSLRAEDTAVYYCTR | LQPEDFATYYCQQFWDNPLTFGQGT |
| | EGAHYDSSYLAWFAHWGQGTLVTV | KLEIKRTVAAPSVFIFPPSDEQLKSGT |
| | SSASTKGPSVFPLAPCSRSTSESTAAL | ASVVCLLNNFYPREAKVQWKVDNA |
| | GCLVKDYFPEPVTVSWNSGALTSGV | LQSGNSQESVTEQDSKDSTYSLSSTL |
| | HTFPAVLQSSGLYSLSSVVTVPSSSL | TLSKADYEKHKVYACEVTHQGLSSP |
| | GTKTYTCNVDHKPSNTKVDKRVES | VTKSFNRGEC (SEQ ID NO: 1148) |
| | KYGPPCPSCPAPEFLGGPSVFLFPPKP | |
| | KDTLMISRTPEVTCVVVDVSQEDPE | |
| | VQFNWYVDGVEVHNAKTKPREEQF | |
| | NSTYRVVSVLTVLHQDWLNGKEYK | |
| | CKVSNKGLPSSIEKTISKAKGQPREP | |
| | QVYTLPPSQEEMTKNQVSLTCLVKG | |
| | FYPSDIAVEWESNGQPENNYKTTPP | |
| | VLDSDGSFFLYSRLTVDKSRWQEGN | |
| | VFSCSVMHEALHNHYTQKSLSLSLG | |
| | K (SEQ ID NO: 1147) | |
| LK-RB2- | QVQLVQSGSELKKPGASVKVSCKAS | EIVLTQSPATLSLSPGERATLSCRASQ |
| 24.h1 | GYTFTTYGMSWVKQAPGQGLKWM | SISDYLHWYQQKPGQAPRLLIKYAS |
| | GWLNIYSGVPAYADDFKGRFVFSLD | QSISGIPARFSGSGSGTDFTLTISSLEP |
| | TSASTAYLQISSLKAEDTAVYFCARR | EDFAVYYCQNGHSFPYTFGGGTKLE |
| | ENYDGYGMDYWGQGTLVTVSSAST | IKRTVAAPSVFIFPPSDEQLKSGTASV |
| | KGPSVFPLAPCSRSTSESTAALGCLV | VCLLNNFYPREAKVQWKVDNALQS |
| | KDYFPEPVTVSWNSGALTSGVHTFP | GNSQESVTEQDSKDSTYSLSSTLTLS |
| | AVLQSSGLYSLSSVVTVPSSSLGTKT | KADYEKHKVYACEVTHQGLSSPVT |
| | YTCNVDHKPSNTKVDKRVESKYGPP | KSFNRGEC (SEQ ID NO: 1150) |
| | CPSCPAPEFLGGPSVFLFPPKPKDTL | |
| | MISRTPEVTCVVVDVSQEDPEVQFN | |
| | WYVDGVEVHNAKTKPREEQFNSTY | |
| | RVVSVLTVLHQDWLNGKEYKCKVS | |
| | NKGLPSSIEKTISKAKGQPREPQVYT | |
| | LPPSQEEMTKNQVSLTCLVKGFYPS | |
| | DIAVEWESNGQPENNYKTTPPVLDS | |
| | DGSFFLYSRLTVDKSRWQEGNVFSC | |
| | SVMHEALHNHYTQKSLSLSLGK | |
| | (SEQ ID NO: 1149) | |
| LK-RB2- | QVQLVQSGSELKKPGASVKVSCKAS | EIVLTQSPATLSLSPGERATLSCRASQ |
| 24.h2 | GYTFTTYGMSWVKQAPGQGLKWM | SISDYLHWYQQKPGQAPRLLIKYAS |
| | GWLNIYSGVPAYADDFKGRFVFSLD | QSISGIPSRFSGSGSGTDFTLTISSLEP |
| | TSASTAYLQISSLKAEDTAVYFCARR | EDFAVYYCQNGHSFPYTFGGGTKLE |
| | ENYDGYGMDYWGQGTLVTVSSAST | IKRTVAAPSVFIFPPSDEQLKSGTASV |
| | KGPSVFPLAPCSRSTSESTAALGCLV | VCLLNNFYPREAKVQWKVDNALQS |
| | KDYFPEPVTVSWNSGALTSGVHTFP | GNSQESVTEQDSKDSTYSLSSTLTLS |
| | AVLQSSGLYSLSSVVTVPSSSLGTKT | KADYEKHKVYACEVTHQGLSSPVT |
| | YTCNVDHKPSNTKVDKRVESKYGPP | KSFNRGEC (SEQ ID NO: 1152) |
| | CPSCPAPEFLGGPSVFLFPPKPKDTL | |
| | MISRTPEVTCVVVDVSQEDPEVQFN | |
| | WYVDGVEVHNAKTKPREEQFNSTY | |
| | RVVSVLTVLHQDWLNGKEYKCKVS | |
| | NKGLPSSIEKTISKAKGQPREPQVYT | |
| | LPPSQEEMTKNQVSLTCLVKGFYPS | |
| | DIAVEWESNGQPENNYKTTPPVLDS | |
| | DGSFFLYSRLTVDKSRWQEGNVFSC | |
| | SVMHEALHNHYTQKSLSLSLGK | |
| | (SEQ ID NO: 1151) | |
| LK-RB2- | QVQLVQSGSELKKPGASVKVSCKAS | EIVLTQSPATLSLSPGERATLSCRASQ |
| 24.h3 | GYTFTTYGMSWVKQAPGQGLKWM | SISDYLHWYQQKPGQAPRLLIKYAS |
| | GWLNIYSGVPAYADDFKGRFVFSLD | QSISGIPSRFSGSGSGTDFTLTISSLEP |
| | TSASTAYLQISSLKAEDTAVYFCARR | EDVAVYYCQNGHSFPYTFGGGTKLE |
| | ENYDGYGMDYWGQGTLVTVSSAST | IKRTVAAPSVFIFPPSDEQLKSGTASV |
| | KGPSVFPLAPCSRSTSESTAALGCLV | VCLLNNFYPREAKVQWKVDNALQS |
| | KDYFPEPVTVSWNSGALTSGVHTFP | GNSQESVTEQDSKDSTYSLSSTLTLS |
| | AVLQSSGLYSLSSVVTVPSSSLGTKT | KADYEKHKVYACEVTHQGLSSPVT |
| | YTCNVDHKPSNTKVDKRVESKYGPP | KSFNRGEC (SEQ ID NO: 1154) |
| | CPSCPAPEFLGGPSVFLFPPKPKDTL | |
| | MISRTPEVTCVVVDVSQEDPEVQFN | |
| | WYVDGVEVHNAKTKPREEQFNSTY | |
| | RVVSVLTVLHQDWLNGKEYKCKVS | |
| | NKGLPSSIEKTISKAKGQPREPQVYT | |
| | LPPSQEEMTKNQVSLTCLVKGFYPS | |
| | DIAVEWESNGQPENNYKTTPPVLDS | |
| | DGSFFLYSRLTVDKSRWQEGNVFSC | |
| | SVMHEALHNHYTQKSLSLSLGK | |
| | (SEQ ID NO: 1153) | |

TABLE 14
VH and VL sequences of humanized antibodies

| Antibody | VH | VL |
|---|---|---|
| LK-RB1-9.h1 | QVQLVQSGSELKKPGASVKVSCKA | DIQMTQSPSSLSASVGDRVTITCETSE |
| | SGYTFTTYGMSWVRQAPGQGLEW | NIYGALNWYQQKPGKAPKLLIYGAT |
| | MGWINTYSGVPTYADDFKGRFVFS | NLAEGVPSRFSGSGSGTDFTFTISSLQ |
| | LDTSVSTAYLQISSLKAEDTAVYYC | PEDIATYYCQNVLNTPWTFGGGTKL |
| | ARRRRTYHGYLSFDYWGQGTTVTV | EIK (SEQ ID NO: 1156) |
| | SS (SEQ ID NO: 1155) | |
| LK-RB1-9.h2 | QVQLVQSGSELKKPGASVKVSCTAS | DIQMTQSPSSLSASVGDRVTITCETSE |
| | GYTFTTYGMSWVRQAPGQGLKWM | NIYGALNWYQQKPGKAPKLLIYGAT |
| | GWINTYSGVPTYADDFKGRFVFSLD | NLAEGVPSRFSGSGSGTDYTFTISSLQ |
| | TSVSTAYLQISSLKAEDTAVYFCAR | PEDIATYYCQNVLNTPWTFGGGTKL |
| | RRRTYHGYLSFDYWGQGTTVTVSS | EIK (SEQ ID NO: 1158) |
| | (SEQ ID NO: 1157) | |
| LK-RB1-9.h3 | QVQLVQSGSELKKPGASVKVSCTAS | DIQMTQSPSSLSASVGDRVTITCETSE |
| | GYTFTTYGMSWVKQAPGQGLKWM | NIYGALNWYQQKPGKAPKLLIYGAT |
| | GWINTYSGVPTYADDFKGRFVFSLD | NLAEGVPSRFSGSGSGTDYTFTISSLQ |
| | TSVSTAYLQISSLKAEDTAVYFCAR | PEDIATYYCQNVLNTPWTFGGGTKL |
| | RRRTYHGYLSFDYWGQGTTVTVSS | EIK (SEQ ID NO: 1160) |
| | (SEQ ID NO: 1159) | |
| LK-RB1- | QVQLQESGPGLVKPSETLSLTCTVS | DIQMTQSPSTLSASVGDRVTITCHAS |
| 16.h1 | GFSLISNAISWIRQPPGKGLEWLGVI | QSVDFWLNWYQQKPGKAPKLLIYK |
| | WGDGNTIYHSALRSRVTISKDTSKN | ASNLHTGVPSRFSGSGSGTEFTLTISS |
| | QFSLKLSSVTAADTAVYYCAKANW | LQPDDFATYYCQQGQSYPLTFGQGT |
| | DGYYFDYWGQGTTVTVSS (SEQ ID | KLEIK (SEQ ID NO: 1162) |
| | NO: 1161) | |
| LK-RB1- | QVQLQESGPGLVKPSETLSLTCTVS | DIQMTQSPSTLSASVGDRVTITCHAS |
| 16.h2 | GFSLISNAISWIRQPPGKGLEWLGVI | QSVDFWLNWYQQKPGKAPKLLIYK |
| | WGDGNTIYHSALRSRLTISKDTSKN | ASNLHTGVPSRFSGSGSGTEFTLTISS |
| | QVFLKLSSVTAADTAVYYCAKANW | LQPDDFATYYCQQGQSYPLTFGQGT |
| | DGYYFDYWGQGTTVTVSS (SEQ ID | KLEIK (SEQ ID NO: 1164) |
| | NO: 1163) | |
| LK-RB1- | QVQLQESGPGLVKPSETLSLTCTVS | DIQMTQSPSTLSASVGDRVTITCHAS |
| 16.h3 | GFSLISNAISWIRQPPGKGLEWLGVI | QSVDFWLNWYQQKPGKAPKLLISKA |
| | WGDGNTIYHSALRSRLTISKDTSKN | SNLHTGVPSRFSGSGSGTEFTLTISSL |
| | QVFLKLSSVTAADTAVYYCAKANW | QPDDFATYYCQQGQSYPLTFGQGTK |
| | DGYYFDYWGQGTTVTVSS (SEQ ID | LEIK (SEQ ID NO: 1166) |
| | NO: 1165) | |
| LK-RB2-2.h1 | EVQLVESGGGLVKPGGSLRLSCAAS | DIQMTQSPSSVSASVGDRVTITCRAS |
| | GFTFSSYAMSWVRQAPGKGLEWVS | ENIYSNLAWYQQKPGKAPKLLIYAA |
| | YISSGGDYFHYADTVKGRFTISRDN | TNLAEGVPSRFSGSGSGTDYTLTISSL |
| | AKNSLYLQMNSLRAEDTAVYYCAR | QPEDFATYYCQQFWDNPLTFGQGTK |
| | EGAHYDSSYLAWFAHWGQGTLVT | LEIK (SEQ ID NO: 1168) |
| | VSS (SEQ ID NO: 1167) | |
| LK-RB2-2.h2 | EVQLVESGGGLVKPGGSLRLSCAAS | DIQMTQSPSSVSASVGDRVTITCRAS |
| | GFTFSSYAMSWVRQAPGKGLEWVS | ENIYSNLAWYQQKPGKAPKLLVYAA |
| | YISSGGDYFHYADTVKGRFTISRDN | TNLAEGVPSRFSGSGSGTDYTLTISSL |
| | AKNSLYLQMNSLRAEDTAVYYCAR | QPEDFATYYCQQFWDNPLTFGQGTK |
| | EGAHYDSSYLAWFAHWGQGTLVT | LEIK (SEQ ID NO: 1170) |
| | VSS (SEQ ID NO: 1169) | |
| LK-RB2-2.h3 | EVQLVESGGGLVKPGGSLRLSCAAS | DIQMTQSPSSVSASVGDRVTITCRAS |
| | GFTFSSYAMSWVRQAPGKGLEWVA | ENIYSNLAWYQQKPGKAPKLLIYAA |
| | YISSGGDYFHYADTVKGRFTISRDN | TNLAEGVPSRFSGSGSGTDYTLTISSL |
| | AKNSLYLQMNSLRAEDTAVYYCTR | QPEDFATYYCQQFWDNPLTFGQGTK |
| | EGAHYDSSYLAWFAHWGQGTLVT | LEIK (SEQ ID NO: 1172) |
| | VSS (SEQ ID NO: 1171) | |
| LK-RB2- | QVQLVQSGSELKKPGASVKVSCKA | EIVLTQSPATLSLSPGERATLSCRASQ |
| 24.h1 | SGYTFTTYGMSWVKQAPGQGLKW | SISDYLHWYQQKPGQAPRLLIKYAS |
| | MGWLNIYSGVPAYADDFKGRFVFS | QSISGIPARFSGSGSGTDFTLTISSLEP |
| | LDTSASTAYLQISSLKAEDTAVYFC | EDFAVYYCQNGHSFPYTFGGGTKLEI |
| | ARRENYDGYGMDYWGQGTLVTVS | K (SEQ ID NO: 1174) |
| | S (SEQ ID NO: 1173) | |
| LK-RB2- | QVQLVQSGSELKKPGASVKVSCKA | EIVLTQSPATLSLSPGERATLSCRASQ |
| 24.h2 | SGYTFTTYGMSWVKQAPGQGLKW | SISDYLHWYQQKPGQAPRLLIKYAS |
| | MGWLNIYSGVPAYADDFKGRFVFS | QSISGIPSRFSGSGSGTDFTLTISSLEPE |
| | LDTSASTAYLQISSLKAEDTAVYFC | DFAVYYCQNGHSFPYTFGGGTKLEI |
| | ARRENYDGYGMDYWGQGTLVTVS | K (SEQ ID NO: 1176) |
| | S (SEQ ID NO: 1175) | |
| LK-RB2- | QVQLVQSGSELKKPGASVKVSCKA | EIVLTQSPATLSLSPGERATLSCRASQ |
| 24.h3 | SGYTFTTYGMSWVKQAPGQGLKW | SISDYLHWYQQKPGQAPRLLIKYAS |
| | MGWLNIYSGVPAYADDFKGRFVFS | QSISGIPSRFSGSGSGTDFTLTISSLEPE |
| | LDTSASTAYLQISSLKAEDTAVYFC | DVAVYYCQNGHSFPYTFGGGTKLEI |
| | ARRENYDGYGMDYWGQGTLVTVS | K (SEQ ID NO: 1178) |
| | S (SEQ ID NO: 1177) | |

TABLE 15
The CDR sequences of humanized antibodies

| Antibody | CDR-H1 | CDR-H2 | CDR-H3 | CDR-L1 | CDR-L2 | CDR-L3 |
|---|---|---|---|---|---|---|
| LK-RB1-9.h1 | TYGMS | WINTYSG | RRRTYHG | ETSENIYG | GATNLAE | QNVLNTP |
| | (SEQ ID | VPTYADD | YLSFDY | ALN (SEQ | (SEQ ID | WT (SEQ |
| | NO: 543) | FKG (SEQ | (SEQ ID | ID NO: | NO: 1281) | ID NO: |
| | | ID NO: | NO: 1179) | 546) | | 548) |
| | | 544) | | | | |
| LK-RB1-9.h2 | TYGMS | WINTYSG | RRRTYHG | ETSENIYG | GATNLAE | QNVLNTP |
| | (SEQ ID | VPTYADD | YLSFDY | ALN (SEQ | (SEQ ID | WT (SEQ |
| | NO: 543) | FKG (SEQ | (SEQ ID | ID NO: | NO: 1281) | ID NO: |
| | | ID NO: | NO: 1179) | 546) | | 548) |
| | | 544) | | | | |
| LK-RB1-9.h3 | TYGMS | WINTYSG | RRRTYHG | ETSENIYG | GATNLAE | QNVLNTP |
| | (SEQ ID | VPTYADD | YLSFDY | ALN (SEQ | (SEQ ID | WT (SEQ |
| | NO: 543) | FKG (SEQ | (SEQ ID | ID NO: | NO: 1281) | ID NO: |
| | | ID NO: | NO: 1179) | 546) | | 548) |
| | | 544) | | | | |
| LK-RB1- | SNAIS | VIWGDGN | ANWDGY | HASQSVD | KASNLHT | QQGQSYP |
| 16.h1 | (SEQ ID | TIYHSALR | YFDY | FWLN | (SEQ ID | LT (SEQ |
| | NO: 585) | S (SEQ ID | (SEQ ID | (SEQ ID | NO: 589) | ID NO: |
| | | NO: 586) | NO: 587) | NO: 588) | | 590) |
| LK-RB1- | SNAIS | VIWGDGN | ANWDGY | HASQSVD | KASNLHT | QQGQSYP |
| 16.h2 | (SEQ ID | TIYHSALR | YFDY | FWLN | (SEQ ID | LT (SEQ |
| | NO: 585) | S (SEQ ID | (SEQ ID | (SEQ ID | NO: 589) | ID NO: |
| | | NO: 586) | NO: 587) | NO: 588) | | 590) |
| LK-RB1- | SNAIS | VIWGDGN | ANWDGY | HASQSVD | KASNLHT | QQGQSYP |
| 16.h3 | (SEQ ID | TIYHSALR | YFDY | FWLN | (SEQ ID | LT (SEQ |
| | NO: 585) | S (SEQ ID | (SEQ ID | (SEQ ID | NO: 589) | ID NO: |
| | | NO: 586) | NO: 587) | NO: 588) | | 590) |
| LK-RB2-2.h1 | SYAMS | YISSGGD | EGAHYDS | RASENIYS | AATNLAE | QQFWDN |
| | (SEQ ID | YFHYADT | SYLAWFA | NLA (SEQ | (SEQ ID | PLT (SEQ |
| | NO: 855) | VKG (SEQ | H (SEQ ID | ID NO: | NO: 1282) | ID NO: |
| | | ID NO: | NO: 857) | 858) | | 860) |
| | | 856) | | | | |
| LK-RB2-2.h2 | SYAMS | YISSGGD | EGAHYDS | RASENIYS | AATNLAE | QQFWDN |
| | (SEQ ID | YFHYADT | SYLAWFA | NLA (SEQ | (SEQ ID | PLT (SEQ |
| | NO: 855) | VKG (SEQ | H (SEQ ID | ID NO: | NO: 1282) | ID NO: |
| | | ID NO: | NO: 857) | 858) | | 860) |
| | | 856) | | | | |
| LK-RB2-2.h3 | SYAMS | YISSGGD | EGAHYDS | RASENIYS | AATNLAE | QQFWDN |
| | (SEQ ID | YFHYADT | SYLAWFA | NLA (SEQ | (SEQ ID | PLT (SEQ |
| | NO: 855) | VKG (SEQ | H (SEQ ID | ID NO: | NO: 1282) | ID NO: |
| | | ID NO: | NO: 857) | 858) | | 860) |
| | | 856) | | | | |
| LK-RB2- | TYGMS | WLNIYSG | RENYDGY | RASQSISD | YASQSIS | QNGHSFP |
| 24.h1 | (SEQ ID | VPAYADD | GMDY | YLH (SEQ | (SEQ ID | YT (SEQ |
| | NO: 987) | FKG (SEQ | (SEQ ID | ID NO: | NO: 991) | ID NO: |
| | | ID NO: | NO: 989) | 990) | | 992) |
| | | 988) | | | | |
| LK-RB2- | TYGMS | WLNIYSG | RENYDGY | RASQSISD | YASQSIS | QNGHSFP |
| 24.h2 | (SEQ ID | VPAYADD | GMDY | YLH (SEQ | (SEQ ID | YT (SEQ |
| | NO: 987) | FKG (SEQ | (SEQ ID | ID NO: | NO: 991) | ID NO: |
| | | ID NO: | NO: 989) | 990) | | 992) |
| | | 988) | | | | |
| LK-RB2- | TYGMS | WLNIYSG | RENYDGY | RASQSISD | YASQSIS | QNGHSFP |
| 24.h3 | (SEQ ID | VPAYADD | GMDY | YLH (SEQ | (SEQ ID | YT (SEQ |
| | NO: 987) | FKG (SEQ | (SEQ ID | ID NO: | NO: 991) | ID NO: |
| | | ID NO: | NO: 989) | 990) | | 992) |
| | | 988) | | | | |

### Affinity Ranking of Chimeric, Humanized Antibodies and PTM Removal Antibodies

For affinity ranking, antibodies were captured on the sensor chip through Fc capture method. LILRB1-his, LILRB2-his or LILRA2-his was used as the analyte. The surface was regenerated before the injection of another antibody. The process was repeated until all antibodies were analyzed. The off-rates were obtained from fitting the experimental data locally to 1:1 interaction model using the Biacore 8K evaluation software. The data of dissociation (kd) and association (ka) rate constants were obtained using Biacore 8K evaluation software. The equilibrium dissociation constants (KD) were calculated from the ratio of kd over ka. The antibodies were ranked by their dissociation rate constant kd. As shown in Table 16, based on the ranking result, the top 3 clones and suitable PTM removal mutation were selected.

TABLE 16
The binding kinetics of humanized antibodies to human LILRB1, human LILRB2 and human LILRA2

| Ligand | Analyte | Chi² (RU²) | ka (1/Ms) | kd (1/s) | KD (M) | Rmax (RU) |
|---|---|---|---|---|---|---|
| LK-RB1-9 | LILRA2 | 1.35E+00 | 2.15E+05 | 2.86E−03 | 1.33E−08 | 7.57E+01 |
| LK-RB1-9.h1 | LILRA2 | 2.26E−01 | 2.10E+05 | 2.19E−03 | 1.04E−08 | 1.11E+02 |
| LK-RB1- | LILRA2 | 7.89E−01 | 1.89E+05 | 1.32E−03 | 6.97E−09 | 1.56E+02 |
| 9.h2 | | | | | | |
| LK-RB1- | LILRA2 | 2.13E−01 | 1.86E+05 | 1.26E−03 | 6.77E−09 | 7.31E+01 |
| 9.h3 | | | | | | |
| LK-RB1-9 | LILRB1 | 4.51E−01 | 3.05E+05 | 2.55E−04 | 8.36E−10 | 9.65E+01 |
| LK-RB1- | LILRB1 | 1.21E+00 | 2.53E+05 | 2.09E−04 | 8.25E−10 | 1.23E+02 |
| 9.h1 | | | | | | |
| LK-RB1- | LILRB1 | 1.45E+00 | 2.41E+05 | 2.24E−04 | 9.27E−10 | 1.79E+02 |
| 9.h2 | | | | | | |
| LK-RB1- | LILRB1 | 4.16E−01 | 2.32E+05 | 1.15E−04 | 4.97E−10 | 8.63E+01 |
| 9.h3 | | | | | | |
| LK-RB1- | LILRB1 | 5.25E−02 | 5.56E+05 | 1.08E−04 | 1.94E−10 | 6.65E+01 |
| 16 | | | | | | |
| LK-RB1- | LILRB1 | 7.22E−02 | 3.94E+05 | 2.00E−04 | 5.07E−10 | 6.87E+01 |
| 16.h1 | | | | | | |
| LK-RB1- | LILRB1 | 6.42E−02 | 4.76E+05 | 1.12E−04 | 2.35E−10 | 5.83E+01 |
| 16.h2 | | | | | | |
| LK-RB1- | LILRB1 | 3.77E−02 | 4.44E+05 | 1.07E−04 | 2.40E−10 | 5.46E+01 |
| 16.h3 | | | | | | |
| LK-RB2-2 | LILRB2 | 5.24E−02 | 8.00E+05 | 1.34E−04 | 1.68E−10 | 5.03E+01 |
| LK-RB2- | LILRB2 | 3.31E−02 | 7.66E+05 | 1.23E−04 | 1.61E−10 | 7.79E+01 |
| 2.h1 | | | | | | |
| LK-RB2- | LILRB2 | 2.90E−02 | 6.73E+05 | 1.40E−04 | 2.08E−10 | 7.48E+01 |
| 2.h2 | | | | | | |
| LK-RB2- | LILRB2 | 2.01E−01 | 6.42E+05 | 1.29E−04 | 2.01E−10 | 1.88E+02 |
| 2.h3 | | | | | | |
| LK-RB2- | LILRB2 | 8.53E−02 | 9.50E+05 | 4.84E−04 | 5.10E−10 | 6.07E+01 |
| 24 | | | | | | |
| LK-RB2- | LILRB2 | 1.92E−02 | 9.07E+05 | 7.58E−04 | 8.36E−10 | 2.39E+01 |
| 24.h1 | | | | | | |
| LK-RB2- | LILRB2 | 2.25E−02 | 8.60E+05 | 8.71E−04 | 1.01E−09 | 1.30E+01 |
| 24.h2 | | | | | | |
| LK-RB2- | LILRB2 | 5.80E−02 | 7.49E+05 | 6.38E−04 | 8.52E−10 | 3.73E+01 |
| 24.h3 | | | | | | |
| LK-RB2- | LILRB1 | 2.87E+00 | 1.92E+05 | 4.06E−03 | 2.11E−08 | 1.26E+02 |
| 24 | | | | | | |
| LK-RB2- | LILRB1 | 2.03E+00 | 1.77E+05 | 4.46E−03 | 2.51E−08 | 1.38E+02 |
| 24.h1 | | | | | | |
| LK-RB2- | LILRB1 | 1.80E+00 | 1.84E+05 | 5.82E−03 | 3.16E−08 | 1.50E+02 |
| 24.h2 | | | | | | |
| LK-RB2- | LILRB1 | 1.11E+00 | 1.67E+05 | 3.35E−03 | 2.01E−08 | 8.79E+01 |
| 24.h3 | | | | | | |

### Humanized Antibodies Characterization

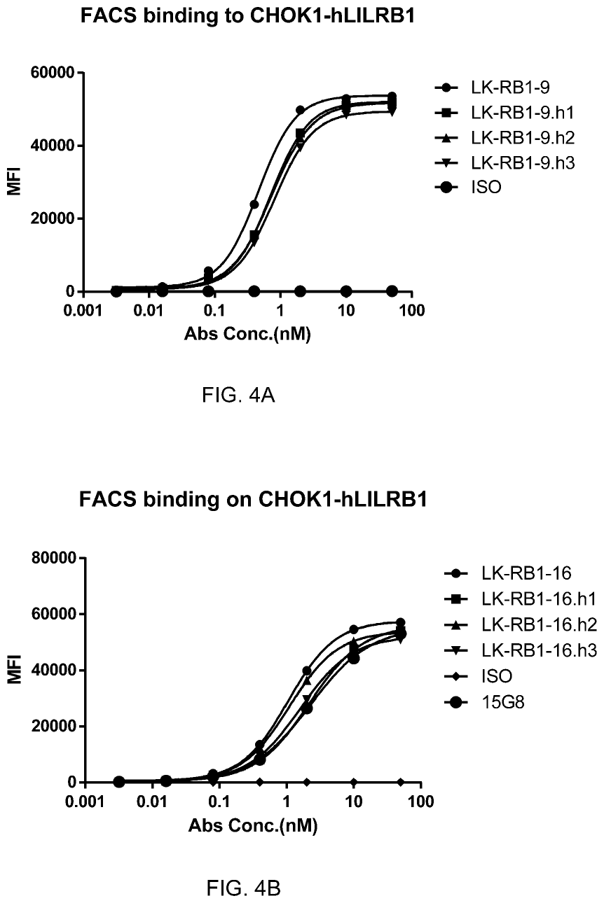

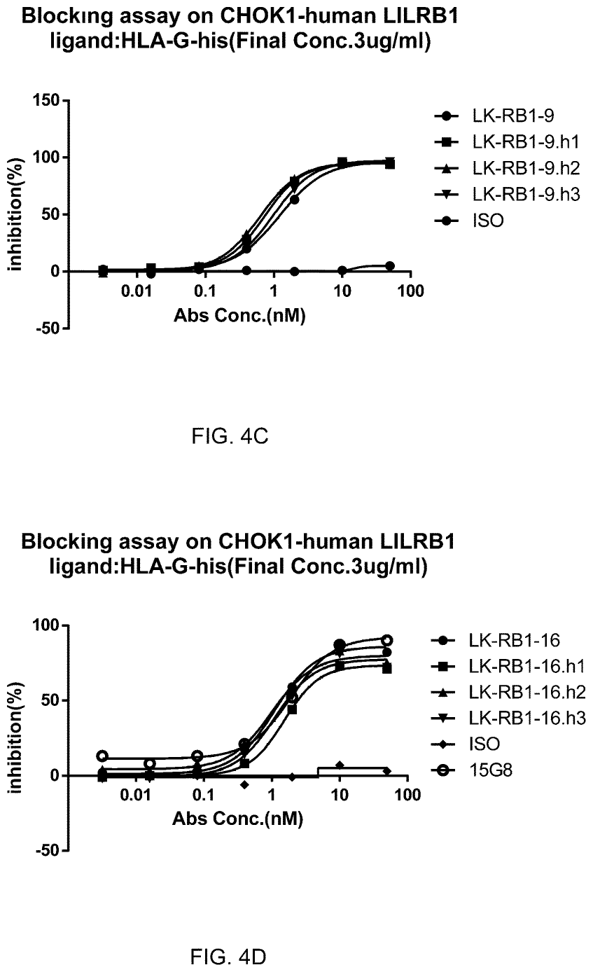

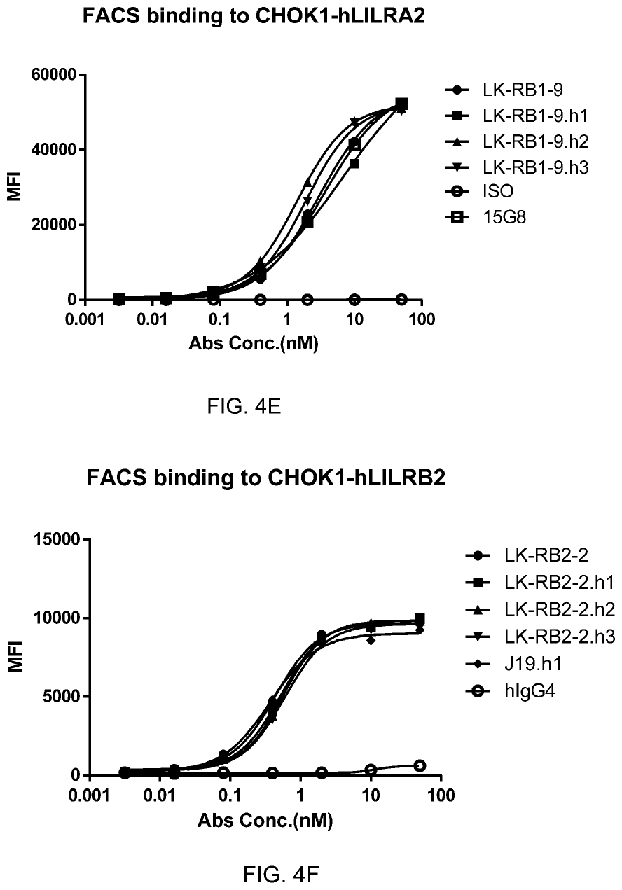

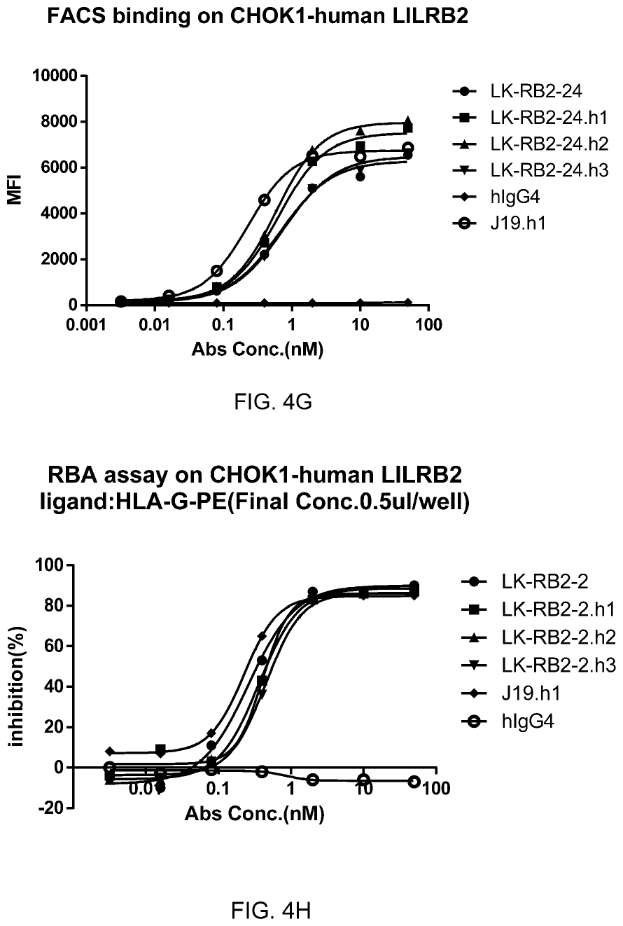

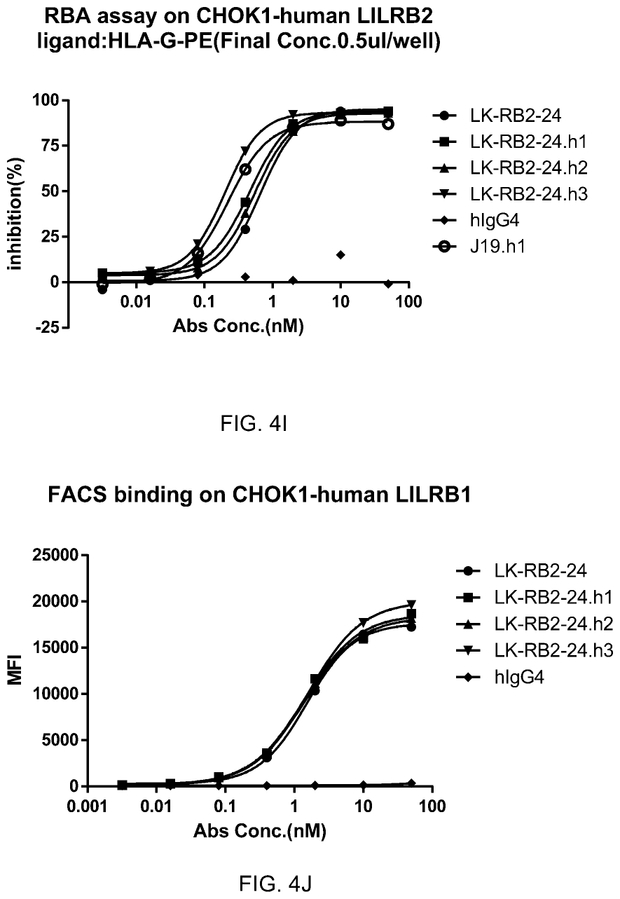

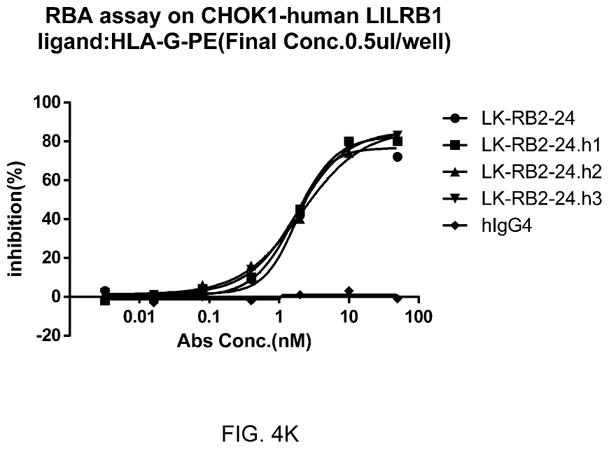

Binding and blocking activity of humanized antibodies were evaluated. As shown in FIGS. 4A-4D, humanized antibodies of LK-RB1-9 and LK-RB1-16 all bind to human LILRB1 and blocked the interaction between human LILRB1 with HLA-G with similar potency comparing to the parental antibody LK-RB1-9 and LKRB1-16. Humanized antibodies of LK-RB1-9 also bind to human LILRA2 (FIG. 4E). Humanized antibodies of LK-RB2-2 and LK-RB2-24 all bind to human LILRB2 and blocked the interaction between human LILRB2 with HLA-G with similar potency comparing to the parental antibody LK-RB2-2 and LK-RB2-24. (FIGS. 4F-4I). Humanized antibodies of LK-RB2-24 also bind to human LILRB1 and blocked the interaction between human LILRB1 with HLA-G (FIGS. 4J-4K).

### 7.3 Example 3: Design and Characterization of Anti-LILRB1/LILRB2 Bispecific Antibodies

### Bispecific Antibody Design

12 anti-LILRB1/LILRB2 bispecific antibodies were designed mainly based on two formats, immunoglobulin-single chain antibody fragment (IgG-ScFv) and dual-variable-domain-immunoglobulin (DVD-Ig). Sequence of anti-LILRB1 antibody (LK-RB1-9.h1, LK-RB1-16.h3) and anti-LILRB2 antibody (LK-RB2-2.h1, LK-RB2-24.h3.) The sequences of bispecific antibodies are shown in Table 17.

Exemplary signal peptides used for antibody expression include without limitation to: MHSSALLCCLVLLTGVRA (SEQ ID NO:1283).

TABLE 17
The sequences of LILRB1 and LILRB2 bispecific antibodies

| Bispecific | | | |
|---|---|---|---|
| antibody | Heavy chain | Light chain | Configuration |
| LK-RBi- | QVQLVQSGSELKKPGAS | DIQMTQSPSSLSASVGDR | IgG-ScFv configuration, |
| 01 | VKVSCKASGYTFTTYGM | VTITCETSENIYGALNWY | two scFv of LK-RB2-24.h3 |
| | SWVRQAPGQGLEWMG | QQKPGKAPKLLIYGATN | each attached to the c- |
| | WINTYSGVPTYADDFKG | LAEGVPSRFSGSGSGTDF | terminus of each of the two |
| | RFVFSLDTSVSTAYLQIS | TFTISSLQPEDIATYYCQ | heavy chains of LK-RB1- |
| | SLKAEDTAVYYCARRRR | NVLNTPWTFGGGTKLEI | 9.h1. |
| | TYHGYLSFDYWGQGTT | KRTVAAPSVFIFPPSDEQ | |
| | VTVSSASTKGPSVFPLAP | LKSGTASVVCLLNNFYP | |
| | CSRSTSESTAALGCLVK | REAKVQWKVDNALQSG | |
| | DYFPEPVTVSWNSGALT | NSQESVTEQDSKDSTYS | |
| | SGVHTFPAVLQSSGLYSL | LSSTLTLSKADYEKHKV | |
| | SSVVTVPSSSLGTKTYTC | YACEVTHQGLSSPVTKS | |
| | NVDHKPSNTKVDKRVES | FNRGEC (SEQ ID NO: | |
| | KYGPPCPPCPAPEFLGGP | 1181) | |
| | SVFLFPPKPKDTLMISRT | | |
| | PEVTCVVVDVSQEDPEV | | |
| | QFNWYVDGVEVHNAKT | | |
| | KPREEQFNSTYRVVSVL | | |
| | TVLHQDWLNGKEYKCK | | |
| | VSNKGLPSSIEKTISKAK | | |
| | GQPREPQVYTLPPSQEE | | |
| | MTKNQVSLTCLVKGFYP | | |
| | SDIAVEWESNGQPENNY | | |
| | KTTPPVLDSDGSFFLYSR | | |
| | LTVDKSRWQEGNVFSCS | | |
| | VMHEALHNHYTQKSLSL | | |
| | SLGGGGSGGGGSGSGGG | | |
| | GSQVQLVQSGSELKKPG | | |
| | ASVKVSCKASGYTFTTY | | |
| | GMSWVKQAPGQGLKW | | |
| | MGWLNIYSGVPAYADD | | |
| | FKGRFVFSLDTSASTAYL | | |
| | QISSLKAEDTAVYFCAR | | |
| | RENYDGYGMDYWGQG | | |
| | TLVTVSSGSGGGGSGSG | | |
| | GGGSGSEIVLTQSPATLS | | |
| | LSPGERATLSCRASQSIS | | |
| | DYLHWYQQKPGQAPRL | | |
| | LIKYASQSISGIPSRFSGS | | |
| | GSGTDFTLTISSLEPEDV | | |
| | AVYYCQNGHSFPYTFGG | | |
| | GTKLEIK (SEQ ID NO: | | |
| | 1180) | | |
| LK-RBi- | QVQLVQSGSELKKPGAS | DIQMTQSPSSLSASVGDR | IgG-ScFv configuration, |
| 02 | VKVSCKASGYTFTTYGM | VTITCETSENIYGALNWY | two scFv of LK-RB2-2.h1 |
| | SWVRQAPGQGLEWMG | QQKPGKAPKLLIYGATN | each attached to the c- |
| | WINTYSGVPTYADDFKG | LAEGVPSRFSGSGSGTDF | terminus of each of the two |
| | RFVFSLDTSVSTAYLQIS | TFTISSLQPEDIATYYCQ | heavy chains of LK-RB1- |
| | SLKAEDTAVYYCARRRR | NVLNTPWTFGGGTKLEI | 9.h1. |
| | TYHGYLSFDYWGQGTT | KRTVAAPSVFIFPPSDEQ | |
| | VTVSSASTKGPSVFPLAP | LKSGTASVVCLLNNFYP | |
| | CSRSTSESTAALGCLVK | REAKVQWKVDNALQSG | |
| | DYFPEPVTVSWNSGALT | NSQESVTEQDSKDSTYS | |
| | SGVHTFPAVLQSSGLYSL | LSSTLTLSKADYEKHKV | |
| | SSVVTVPSSSLGTKTYTC | YACEVTHQGLSSPVTKS | |
| | NVDHKPSNTKVDKRVES | FNRGEC (SEQ ID NO: | |
| | KYGPPCPPCPAPEFLGGP | 1183) | |
| | SVFLFPPKPKDTLMISRT | | |
| | PEVTCVVVDVSQEDPEV | | |
| | QFNWYVDGVEVHNAKT | | |
| | KPREEQFNSTYRVVSVL | | |
| | TVLHQDWLNGKEYKCK | | |
| | VSNKGLPSSIEKTISKAK | | |
| | GQPREPQVYTLPPSQEE | | |
| | MTKNQVSLTCLVKGFYP | | |
| | SDIAVEWESNGQPENNY | | |
| | KTTPPVLDSDGSFFLYSR | | |
| | LTVDKSRWQEGNVFSCS | | |
| | VMHEALHNHYTQKSLSL | | |
| | SLGGGGSGGGGSGSGGG | | |
| | GSEVQLVESGGGLVKPG | | |
| | GSLRLSCAASGFTFSSYA | | |
| | MSWVRQAPGKGLEWVS | | |
| | YISSGGDYFHYADTVKG | | |
| | RFTISRDNAKNSLYLQM | | |
| | NSLRAEDTAVYYCAREG | | |
| | AHYDSSYLAWFAHWGQ | | |
| | GTLVTVSSGSGGGGSGS | | |
| | GGGGSGSDIQMTQSPSS | | |
| | VSASVGDRVTITCRASE | | |
| | NIYSNLAWYQQKPGKAP | | |
| | KLLIYAATNLAEGVPSRF | | |
| | SGSGSGTDYTLTISSLQP | | |
| | EDFATYYCQQFWDNPLT | | |
| | FGQGTKLEIK (SEQ ID | | |
| | NO: 1182) | | |
| LK-RBi- | EVQLVESGGGLVKPGGS | DIQMTQSPSSVSASVGD | IgG-ScFv configuration, |
| 03 | LRLSCAASGFTFSSYAM | RVTITCRASENIYSNLAW | two scFv of LK-RB1-9.h1 |
| | SWVRQAPGKGLEWVSYI | YQQKPGKAPKLLIYAAT | each attached to the c- |
| | SSGGDYFHYADTVKGRF | NLAEGVPSRFSGSGSGT | terminus of each of the two |
| | TISRDNAKNSLYLQMNS | DYTLTISSLQPEDFATYY | heavy chains of LK-RB2- |
| | LRAEDTAVYYCAREGA | CQQFWDNPLTFGQGTKL | 2.h1. |
| | HYDSSYLAWFAHWGQG | EIKRTVAAPSVFIFPPSDE | |
| | TLVTVSSASTKGPSVFPL | QLKSGTASVVCLLNNFY | |
| | APCSRSTSESTAALGCLV | PREAKVQWKVDNALQS | |
| | KDYFPEPVTVSWNSGAL | GNSQESVTEQDSKDSTY | |
| | TSGVHTFPAVLQSSGLYS | SLSSTLTLSKADYEKHK | |
| | LSSVVTVPSSSLGTKTYT | VYACEVTHQGLSSPVTK | |
| | CNVDHKPSNTKVDKRV | SFNRGEC (SEQ ID NO: | |
| | ESKYGPPCPPCPAPEFLG | 1185) | |
| | GPSVFLFPPKPKDTLMIS | | |
| | RTPEVTCVVVDVSQEDP | | |
| | EVQFNWYVDGVEVHNA | | |
| | KTKPREEQFNSTYRVVS | | |
| | VLTVLHQDWLNGKEYK | | |
| | CKVSNKGLPSSIEKTISK | | |
| | AKGQPREPQVYTLPPSQ | | |
| | EEMTKNQVSLTCLVKGF | | |
| | YPSDIAVEWESNGQPEN | | |
| | NYKTTPPVLDSDGSFFLY | | |
| | SRLTVDKSRWQEGNVFS | | |
| | CSVMHEALHNHYTQKS | | |
| | LSLSLGGGGSGGGGSGS | | |
| | GGGGSQVQLVQSGSELK | | |
| | KPGASVKVSCKASGYTF | | |
| | TTYGMSWVRQAPGQGL | | |
| | EWMGWINTYSGVPTYA | | |
| | DDFKGRFVFSLDTSVST | | |
| | AYLQISSLKAEDTAVYY | | |
| | CARRRRTYHGYLSFDY | | |
| | WGQGTTVTVSSGSGGG | | |
| | GSGSGGGGSGSDIQMTQ | | |
| | SPSSLSASVGDRVTITCE | | |
| | TSENIYGALNWYQQKPG | | |
| | KAPKLLIYGATNLAEGV | | |
| | PSRFSGSGSGTDFTFTISS | | |
| | LQPEDIATYYCQNVLNT | | |
| | PWTFGGGTKLEIK (SEQ | | |
| | ID NO: 1184) | | |
| LK-RBi- | QVQLVQSGSELKKPGAS | EIVLTQSPATLSLSPGER | IgG-ScFv configuration, |
| 04 | VKVSCKASGYTFTTYGM | ATLSCRASQSISDYLHW | two scFv of LK-RB1-9.h1 |
| | SWVKQAPGQGLKWMG | YQQKPGQAPRLLIKYAS | each attached to the c- |
| | WLNIYSGVPAYADDFKG | QSISGIPSRFSGSGSGTDF | terminus of each of the two |
| | RFVFSLDTSASTAYLQIS | TLTISSLEPEDVAVYYCQ | heavy chains of LK-RB2- |
| | SLKAEDTAVYFCARREN | NGHSFPYTFGGGTKLEIK | 24.h3. |
| | YDGYGMDYWGQGTLV | RTVAAPSVFIFPPSDEQL | |
| | TVSSASTKGPSVFPLAPC | KSGTASVVCLLNNFYPR | |
| | SRSTSESTAALGCLVKD | EAKVQWKVDNALQSGN | |
| | YFPEPVTVSWNSGALTS | SQESVTEQDSKDSTYSLS | |
| | GVHTFPAVLQSSGLYSLS | STLTLSKADYEKHKVYA | |
| | SVVTVPSSSLGTKTYTCN | CEVTHQGLSSPVTKSFN | |
| | VDHKPSNTKVDKRVESK | RGEC (SEQ ID NO: 1187) | |
| | YGPPCPPCPAPEFLGGPS | | |
| | VFLFPPKPKDTLMISRTP | | |
| | EVTCVVVDVSQEDPEVQ | | |
| | FNWYVDGVEVHNAKTK | | |
| | PREEQFNSTYRVVSVLT | | |
| | VLHQDWLNGKEYKCKV | | |
| | SNKGLPSSIEKTISKAKG | | |
| | QPREPQVYTLPPSQEEM | | |
| | TKNQVSLTCLVKGFYPS | | |
| | DIAVEWESNGQPENNYK | | |
| | TTPPVLDSDGSFFLYSRL | | |
| | TVDKSRWQEGNVFSCSV | | |
| | MHEALHNHYTQKSLSLS | | |
| | LGGGGSGGGGSGSGGG | | |
| | GSQVQLVQSGSELKKPG | | |
| | ASVKVSCKASGYTFTTY | | |
| | GMSWVRQAPGQGLEW | | |
| | MGWINTYSGVPTYADDF | | |
| | KGRFVFSLDTSVSTAYL | | |
| | QISSLKAEDTAVYYCAR | | |
| | RRRTYHGYLSFDYWGQ | | |
| | GTTVTVSSGSGGGGSGS | | |
| | GGGGSGSDIQMTQSPSSL | | |
| | SASVGDRVTITCETSENI | | |
| | YGALNWYQQKPGKAPK | | |
| | LLIYGATNLAEGVPSRFS | | |
| | GSGSGTDFTFTISSLQPE | | |
| | DIATYYCQNVLNTPWTF | | |
| | GGGTKLEIK (SEQ ID | | |
| | NO: 1186) | | |
| LK-RBi- | QVQLVQSGSELKKPGAS | DIQMTQSPSSLSASVGDR | Dual Variable Domain |
| 05 | VKVSCKASGYTFTTYGM | VTITCETSENIYGALNWY | (DVD)-Ig configuration. |
| | SWVRQAPGQGLEWMG | QQKPGKAPKLLIYGATN | |
| | WINTYSGVPTYADDFKG | LAEGVPSRFSGSGSGTDF | |
| | RFVFSLDTSVSTAYLQIS | TFTISSLQPEDIATYYCQ | |
| | SLKAEDTAVYYCARRRR | NVLNTPWTFGGGTKLEI | |
| | TYHGYLSFDYWGQGTT | KGGSGGGGSGDIQMTQS | |
| | VTVSSGGSGGGGSGEVQ | PSSVSASVGDRVTITCRA | |
| | LVESGGGLVKPGGSLRL | SENIYSNLAWYQQKPGK | |
| | SCAASGFTFSSYAMSWV | APKLLIYAATNLAEGVPS | |
| | RQAPGKGLEWVSYISSG | RFSGSGSGTDYTLTISSL | |
| | GDYFHYADTVKGRFTIS | QPEDFATYYCQQFWDNP | |
| | RDNAKNSLYLQMNSLR | LTFGQGTKLEIKRTVAAP | |
| | AEDTAVYYCAREGAHY | SVFIFPPSDEQLKSGTAS | |
| | DSSYLAWFAHWGQGTL | VVCLLNNFYPREAKVQ | |
| | VTVSSASTKGPSVFPLAP | WKVDNALQSGNSQESV | |
| | CSRSTSESTAALGCLVK | TEQDSKDSTYSLSSTLTL | |
| | DYFPEPVTVSWNSGALT | SKADYEKHKVYACEVT | |
| | SGVHTFPAVLQSSGLYSL | HQGLSSPVTKSFNRGEC | |
| | SSVVTVPSSSLGTKTYTC | (SEQ ID NO: 1189) | |
| | NVDHKPSNTKVDKRVES | | |
| | KYGPPCPPCPAPEFLGGP | | |
| | SVFLFPPKPKDTLMISRT | | |
| | PEVTCVVVDVSQEDPEV | | |
| | QFNWYVDGVEVHNAKT | | |
| | KPREEQFNSTYRVVSVL | | |
| | TVLHQDWLNGKEYKCK | | |
| | VSNKGLPSSIEKTISKAK | | |
| | GQPREPQVYTLPPSQEE | | |
| | MTKNQVSLTCLVKGFYP | | |
| | SDIAVEWESNGQPENNY | | |
| | KTTPPVLDSDGSFFLYSR | | |
| | LTVDKSRWQEGNVFSCS | | |
| | VMHEALHNHYTQKSLSL | | |
| | SLGK (SEQ ID NO: 1188) | | |
| LK-RBi- | QVQLQESGPGLVKPSET | DIQMTQSPSTLSASVGDR | IgG-ScFv configuration, |
| 06 | LSLTCTVSGFSLISNAISW | VTITCHASQSVDFWLNW | two scFv of LK-RB2-2.h1 |
| | IRQPPGKGLEWLGVIWG | YQQKPGKAPKLLISKAS | each attached to the c- |
| | DGNTIYHSALRSRLTISK | NLHTGVPSRFSGSGSGTE | terminus of each of the two |
| | DTSKNQVFLKLSSVTAA | FTLTISSLQPDDFATYYC | heavy chains of LK-RB1- |
| | DTAVYYCAKANWDGY | QQGQSYPLTFGQGTKLEI | 16.h3. |
| | YFDYWGQGTTVTVSSAS | KRTVAAPSVFIFPPSDEQ | |
| | TKGPSVFPLAPCSRSTSE | LKSGTASVVCLLNNFYP | |
| | STAALGCLVKDYFPEPV | REAKVQWKVDNALQSG | |
| | TVSWNSGALTSGVHTFP | NSQESVTEQDSKDSTYS | |
| | AVLQSSGLYSLSSVVTVP | LSSTLTLSKADYEKHKV | |
| | SSSLGTKTYTCNVDHKP | YACEVTHQGLSSPVTKS | |
| | SNTKVDKRVESKYGPPC | FNRGEC (SEQ ID NO: | |
| | PPCPAPEFLGGPSVFLFPP | 1191) | |
| | KPKDTLMISRTPEVTCV | | |
| | VVDVSQEDPEVQFNWY | | |
| | VDGVEVHNAKTKPREE | | |
| | QFNSTYRVVSVLTVLHQ | | |
| | DWLNGKEYKCKVSNKG | DIQMTQSPSTLSASVGDR | |
| | LPSSIEKTISKAKGQPREP | VTITCHASQSVDFWLNW | |
| | QVYTLPPSQEEMTKNQV | YQQKPGKAPKLLISKAS | |
| | SLTCLVKGFYPSDIAVE | NLHTGVPSRFSGSGSGTE | |
| | WESNGQPENNYKTTPPV | FTLTISSLQPDDFATYYC | |
| | LDSDGSFFLYSRLTVDKS | QQGQSYPLTFGQGTKLEI | |
| | RWQEGNVFSCSVMHEA | KRTVAAPSVFIFPPSDEQ | |
| | LHNHYTQKSLSLSLGKG | | |
| | GGGSGGGGSGGGGSGG | | |
| | GGSEVQLVESGGGLVKP | | |
| | GGSLRLSCAASGFTFSSY | | |
| | AMSWVRQAPGKGLEWV | | |
| | SYISSGGDYFHYADTVK | | |
| | GRFTISRDNAKNSLYLQ | | |
| | MNSLRAEDTAVYYCAR | | |
| | EGAHYDSSYLAWFAHW | | |
| | GQGTLVTVSSGGGGSGG | | |
| | GGSGGGGSDIQMTQSPS | | |
| | SVSASVGDRVTITCRASE | | |
| | NIYSNLAWYQQKPGKAP | | |
| | KLLIYAATNLAEGVPSRF | | |
| | SGSGSGTDYTLTISSLQP | | |
| | EDFATYYCQQFWDNPLT | | |
| | FGQGTKLEIK (SEQ ID | | |
| | NO: 1190) | | |
| LK-RBi- | QVQLQESGPGLVKPSET | LKSGTASVVCLLNNFYP | IgG-ScFv configuration, |
| 07 | LSLTCTVSGFSLISNAISW | REAKVQWKVDNALQSG | two scFv of LK-RB2-24.h3 |
| | IRQPPGKGLEWLGVIWG | NSQESVTEQDSKDSTYS | each attached to the c- |
| | DGNTIYHSALRSRLTISK | LSSTLTLSKADYEKHKV | terminus of each of the two |
| | DTSKNQVFLKLSSVTAA | YACEVTHQGLSSPVTKS | heavy chains of LK-RB1- |
| | DTAVYYCAKANWDGY | FNRGEC (SEQ ID NO: | 16.h3. |
| | YFDYWGQGTTVTVSSAS | 1193) | |
| | TKGPSVFPLAPCSRSTSE | | |
| | STAALGCLVKDYFPEPV | | |
| | TVSWNSGALTSGVHTFP | | |
| | AVLQSSGLYSLSSVVTVP | | |
| | SSSLGTKTYTCNVDHKP | | |
| | SNTKVDKRVESKYGPPC | | |
| | PPCPAPEFLGGPSVFLFPP | | |
| | KPKDTLMISRTPEVTCV | | |
| | VVDVSQEDPEVQFNWY | | |
| | VDGVEVHNAKTKPREE | | |
| | QFNSTYRVVSVLTVLHQ | | |
| | DWLNGKEYKCKVSNKG | | |
| | LPSSIEKTISKAKGQPREP | | |
| | QVYTLPPSQEEMTKNQV | | |
| | SLTCLVKGFYPSDIAVE | | |
| | WESNGQPENNYKTTPPV | | |
| | LDSDGSFFLYSRLTVDKS | | |
| | RWQEGNVFSCSVMHEA | | |
| | LHNHYTQKSLSLSLGKG | | |
| | GGGSGGGGSGGGGSGG | | |
| | GGSQVQLVQSGSELKKP | | |
| | GASVKVSCKASGYTFTT | | |
| | YGMSWVKQAPGQGLK | | |
| | WMGWLNIYSGVPAYAD | | |
| | DFKGRFVFSLDTSASTA | | |
| | YLQISSLKAEDTAVYFC | | |
| | ARRENYDGYGMDYWG | | |
| | QGTLVTVSSGGGGSGGG | | |
| | GSGGGGSEIVLTQSPATL | | |
| | SLSPGERATLSCRASQSIS | | |
| | DYLHWYQQKPGQAPRL | | |
| | LIKYASQSISGIPSRFSGS | | |
| | GSGTDFTLTISSLEPEDV | | |
| | AVYYCQNGHSFPYTFGG | | |
| | GTKLEIK (SEQ ID NO: | | |
| | 1192) | | |
| LK-RBi- | EVQLVESGGGLVKPGGS | DIQMTQSPSSVSASVGD | IgG-ScFv configuration, |
| 08 | LRLSCAASGFTFSSYAM | RVTITCRASENIYSNLAW | two scFv of LK-RB1-16.h3 |
| | SWVRQAPGKGLEWVSYI | YQQKPGKAPKLLIYAAT | each attached to the c- |
| | SSGGDYFHYADTVKGRF | NLAEGVPSRFSGSGSGT | terminus of each of the two |
| | TISRDNAKNSLYLQMNS | DYTLTISSLQPEDFATYY | heavy chains of LK-RB2- |
| | LRAEDTAVYYCAREGA | CQQFWDNPLTFGQGTKL | 2.h1. |
| | HYDSSYLAWFAHWGQG | EIKRTVAAPSVFIFPPSDE | |
| | TLVTVSSASTKGPSVFPL | QLKSGTASVVCLLNNFY | |
| | APCSRSTSESTAALGCLV | PREAKVQWKVDNALQS | |
| | KDYFPEPVTVSWNSGAL | GNSQESVTEQDSKDSTY | |
| | TSGVHTFPAVLQSSGLYS | SLSSTLTLSKADYEKHK | |
| | LSSVVTVPSSSLGTKTYT | VYACEVTHQGLSSPVTK | |
| | CNVDHKPSNTKVDKRV | SFNRGEC (SEQ ID NO: | |
| | ESKYGPPCPPCPAPEFLG | 1195) | |
| | GPSVFLFPPKPKDTLMIS | | |
| | RTPEVTCVVVDVSQEDP | | |
| | EVQFNWYVDGVEVHNA | | |
| | KTKPREEQFNSTYRVVS | | |
| | VLTVLHQDWLNGKEYK | | |
| | CKVSNKGLPSSIEKTISK | | |
| | AKGQPREPQVYTLPPSQ | | |
| | EEMTKNQVSLTCLVKGF | | |
| | YPSDIAVEWESNGQPEN | | |
| | NYKTTPPVLDSDGSFFLY | | |
| | SRLTVDKSRWQEGNVFS | | |
| | CSVMHEALHNHYTQKS | | |
| | LSLSLGGGGSGGGGSGS | | |
| | GGGGSDIQMTQSPSTLS | | |
| | ASVGDRVTITCHASQSV | | |
| | DFWLNWYQQKPGKAPK | | |
| | LLISKASNLHTGVPSRFS | | |
| | GSGSGTEFTLTISSLQPD | | |
| | DFATYYCQQGQSYPLTF | | |
| | GQGTKLEIKGSGGGGSG | | |
| | SGGGGSGSQVQLQESGP | | |
| | GLVKPSETLSLTCTVSGF | | |
| | SLISNAISWIRQPPGKGLE | | |
| | WLGVIWGDGNTIYHSAL | | |
| | RSRLTISKDTSKNQVFLK | | |
| | LSSVTAADTAVYYCAKA | | |
| | NWDGYYFDYWGQGTT | | |
| | VTVSS (SEQ ID NO: | | |
| | 1194) | | |
| LK-RBi- | QVQLVQSGSELKKPGAS | DIQMTQSPSSLSASVGDR | DVD-Ig configuration. |
| 09 | VKVSCKASGYTFTTYGM | VTITCETSENIYGALNWY | |
| | SWVRQAPGQGLEWMG | QQKPGKAPKLLIYGATN | |
| | WINTYSGVPTYADDFKG | LAEGVPSRFSGSGSGTDF | |
| | RFVFSLDTSVSTAYLQIS | TFTISSLQPEDIATYYCQ | |
| | SLKAEDTAVYYCARRRR | NVLNTPWTFGGGTKLEI | |
| | TYHGYLSFDYWGQGTT | KGGSGGGGSGEIVLTQS | |
| | VTVSSGGSGGGGSGQVQ | PATLSLSPGERATLSCRA | |
| | LVQSGSELKKPGASVKV | SQSISDYLHWYQQKPGQ | |
| | SCKASGYTFTTYGMSW | APRLLIKYASQSISGIPSR | |
| | VKQAPGQGLKWMGWL | FSGSGSGTDFTLTISSLEP | |
| | NIYSGVPAYADDFKGRF | EDVAVYYCQNGHSFPYT | |
| | VFSLDTSASTAYLQISSL | FGGGTKLEIKRTVAAPS | |
| | KAEDTAVYFCARRENY | VFIFPPSDEQLKSGTASV | |
| | DGYGMDYWGQGTLVT | VCLLNNFYPREAKVQW | |
| | VSSASTKGPSVFPLAPCS | KVDNALQSGNSQESVTE | |
| | RSTSESTAALGCLVKDY | QDSKDSTYSLSSTLTLSK | |
| | FPEPVTVSWNSGALTSG | ADYEKHKVYACEVTHQ | |
| | VHTFPAVLQSSGLYSLSS | GLSSPVTKSFNRGEC | |
| | VVTVPSSSLGTKTYTCN | (SEQ ID NO: 1197) | |
| | VDHKPSNTKVDKRVESK | | |
| | YGPPCPPCPAPEFLGGPS | | |
| | VFLFPPKPKDTLMISRTP | | |
| | EVTCVVVDVSQEDPEVQ | | |
| | FNWYVDGVEVHNAKTK | | |
| | PREEQFNSTYRVVSVLT | | |
| | VLHQDWLNGKEYKCKV | | |
| | SNKGLPSSIEKTISKAKG | | |
| | QPREPQVYTLPPSQEEM | | |
| | TKNQVSLTCLVKGFYPS | | |
| | DIAVEWESNGQPENNYK | | |
| | TTPPVLDSDGSFFLYSRL | | |
| | TVDKSRWQEGNVFSCSV | | |
| | MHEALHNHYTQKSLSLS | | |
| | LGK (SEQ ID NO: 1196) | | |
| LK-RBi- | QVQLVQSGSELKKPGAS | EIVLTQSPATLSLSPGER | DVD-Ig configuration. |
| 10 | VKVSCKASGYTFTTYGM | ATLSCRASQSISDYLHW | |
| | SWVKQAPGQGLKWMG | YQQKPGQAPRLLIKYAS | |
| | WLNIYSGVPAYADDFKG | QSISGIPSRFSGSGSGTDF | |
| | RFVFSLDTSASTAYLQIS | TLTISSLEPEDVAVYYCQ | |
| | SLKAEDTAVYFCARREN | NGHSFPYTFGGGTKLEIK | |
| | YDGYGMDYWGQGTLV | GGSGGGGSGDIQMTQSP | |
| | TVSSGGSGGGGSGQVQL | SSLSASVGDRVTITCETS | |
| | VQSGSELKKPGASVKVS | ENIYGALNWYQQKPGK | |
| | CKASGYTFTTYGMSWV | APKLLIYGATNLAEGVPS | |
| | RQAPGQGLEWMGWINT | RFSGSGSGTDFTFTISSLQ | |
| | YSGVPTYADDFKGRFVF | PEDIATYYCQNVLNTPW | |
| | SLDTSVSTAYLQISSLKA | TFGGGTKLEIKRTVAAPS | |
| | EDTAVYYCARRRRTYH | VFIFPPSDEQLKSGTASV | |
| | GYLSFDYWGQGTTVTVS | VCLLNNFYPREAKVQW | |
| | SASTKGPSVFPLAPCSRS | KVDNALQSGNSQESVTE | |
| | TSESTAALGCLVKDYFP | QDSKDSTYSLSSTLTLSK | |
| | EPVTVSWNSGALTSGVH | ADYEKHKVYACEVTHQ | |
| | TFPAVLQSSGLYSLSSVV | GLSSPVTKSFNRGEC | |
| | TVPSSSLGTKTYTCNVD | (SEQ ID NO: 1199) | |
| | HKPSNTKVDKRVESKYG | | |
| | PPCPPCPAPEFLGGPSVF | | |
| | LFPPKPKDTLMISRTPEV | | |
| | TCVVVDVSQEDPEVQFN | | |
| | WYVDGVEVHNAKTKPR | | |
| | EEQFNSTYRVVSVLTVL | | |
| | HQDWLNGKEYKCKVSN | | |
| | KGLPSSIEKTISKAKGQP | | |
| | REPQVYTLPPSQEEMTK | | |
| | NQVSLTCLVKGFYPSDIA | | |
| | VEWESNGQPENNYKTTP | | |
| | PVLDSDGSFFLYSRLTVD | | |
| | KSRWQEGNVFSCSVMH | | |
| | EALHNHYTQKSLSLSLG | | |
| | K (SEQ ID NO: 1198) | | |
| LK-RBi- | QVQLQESGPGLVKPSET | DIQMTQSPSTLSASVGDR | DVD-Ig configuration. |
| 11 | LSLTCTVSGFSLISNAISW | VTITCHASQSVDFWLNW | |
| | IRQPPGKGLEWLGVIWG | YQQKPGKAPKLLISKAS | |
| | DGNTIYHSALRSRLTISK | NLHTGVPSRFSGSGSGTE | |
| | DTSKNQVFLKLSSVTAA | FTLTISSLQPDDFATYYC | |
| | DTAVYYCAKANWDGY | QQGQSYPLTFGQGTKLEI | |
| | YFDYWGQGTTVTVSSG | KGGSGGGGSGDIQMTQS | |
| | GSGGGGSGEVQLVESGG | PSSVSASVGDRVTITCRA | |
| | GLVKPGGSLRLSCAASG | SENIYSNLAWYQQKPGK | |
| | FTFSSYAMSWVRQAPGK | APKLLIYAATNLAEGVPS | |
| | GLEWVSYISSGGDYFHY | RFSGSGSGTDYTLTISSL | |
| | ADTVKGRFTISRDNAKN | QPEDFATYYCQQFWDNP | |
| | SLYLQMNSLRAEDTAVY | LTFGQGTKLEIKRTVAAP | |
| | YCAREGAHYDSSYLAW | SVFIFPPSDEQLKSGTAS | |
| | FAHWGQGTLVTVSSAST | VVCLLNNFYPREAKVQ | |
| | KGPSVFPLAPCSRSTSES | WKVDNALQSGNSQESV | |
| | TAALGCLVKDYFPEPVT | TEQDSKDSTYSLSSTLTL | |
| | VSWNSGALTSGVHTFPA | SKADYEKHKVYACEVT | |
| | VLQSSGLYSLSSVVTVPS | HQGLSSPVTKSFNRGEC | |
| | SSLGTKTYTCNVDHKPS | (SEQ ID NO: 1201) | |
| | NTKVDKRVESKYGPPCP | | |
| | PCPAPEFLGGPSVFLFPP | | |
| | KPKDTLMISRTPEVTCV | | |
| | VVDVSQEDPEVQFNWY | | |
| | VDGVEVHNAKTKPREE | | |
| | QFNSTYRVVSVLTVLHQ | | |
| | DWLNGKEYKCKVSNKG | | |
| | LPSSIEKTISKAKGQPREP | | |
| | QVYTLPPSQEEMTKNQV | | |
| | SLTCLVKGFYPSDIAVE | | |
| | WESNGQPENNYKTTPPV | | |
| | LDSDGSFFLYSRLTVDKS | | |
| | RWQEGNVFSCSVMHEA | | |
| | LHNHYTQKSLSLSLGK | | |
| | (SEQ ID NO: 1200) | | |
| LK-RBi- | QVQLQESGPGLVKPSET | DIQMTQSPSTLSASVGDR | DVD-Ig configuration. |
| 12 | LSLTCTVSGFSLISNAISW | VTITCHASQSVDFWLNW | |
| | IRQPPGKGLEWLGVIWG | YQQKPGKAPKLLISKAS | |
| | DGNTIYHSALRSRLTISK | NLHTGVPSRFSGSGSGTE | |
| | DTSKNQVFLKLSSVTAA | FTLTISSLQPDDFATYYC | |
| | DTAVYYCAKANWDGY | QQGQSYPLTFGQGTKLEI | |
| | YFDYWGQGTTVTVSSG | KGGSGGGGSGEIVLTQS | |
| | GSGGGGSGQVQLVQSGS | PATLSLSPGERATLSCRA | |
| | ELKKPGASVKVSCKASG | SQSISDYLHWYQQKPGQ | |
| | YTFTTYGMSWVKQAPG | APRLLIKYASQSISGIPSR | |
| | QGLKWMGWLNIYSGVP | FSGSGSGTDFTLTISSLEP | |
| | AYADDFKGRFVFSLDTS | EDVAVYYCQNGHSFPYT | |
| | ASTAYLQISSLKAEDTAV | FGGGTKLEIKRTVAAPS | |
| | YFCARRENYDGYGMDY | VFIFPPSDEQLKSGTASV | |
| | WGQGTLVTVSSASTKGP | VCLLNNFYPREAKVQW | |
| | SVFPLAPCSRSTSESTAA | KVDNALQSGNSQESVTE | |
| | LGCLVKDYFPEPVTVSW | QDSKDSTYSLSSTLTLSK | |
| | NSGALTSGVHTFPAVLQ | ADYEKHKVYACEVTHQ | |
| | SSGLYSLSSVVTVPSSSL | GLSSPVTKSFNRGEC | |
| | GTKTYTCNVDHKPSNTK | (SEQ ID NO: 1203) | |
| | VDKRVESKYGPPCPPCP | | |
| | APEFLGGPSVFLFPPKPK | | |
| | DTLMISRTPEVTCVVVD | | |
| | VSQEDPEVQFNWYVDG | | |
| | VEVHNAKTKPREEQFNS | | |
| | TYRVVSVLTVLHQDWL | | |
| | NGKEYKCKVSNKGLPSS | | |
| | IEKTISKAKGQPREPQVY | | |
| | TLPPSQEEMTKNQVSLT | | |
| | CLVKGFYPSDIAVEWES | | |
| | NGQPENNYKTTPPVLDS | | |
| | DGSFFLYSRLTVDKSRW | | |
| | QEGNVFSCSVMHEALHN | | |
| | HYTQKSLSLSLGK (SEQ | | |
| | ID NO: 1202) | | |

### Bispecific Antibodies Characterization

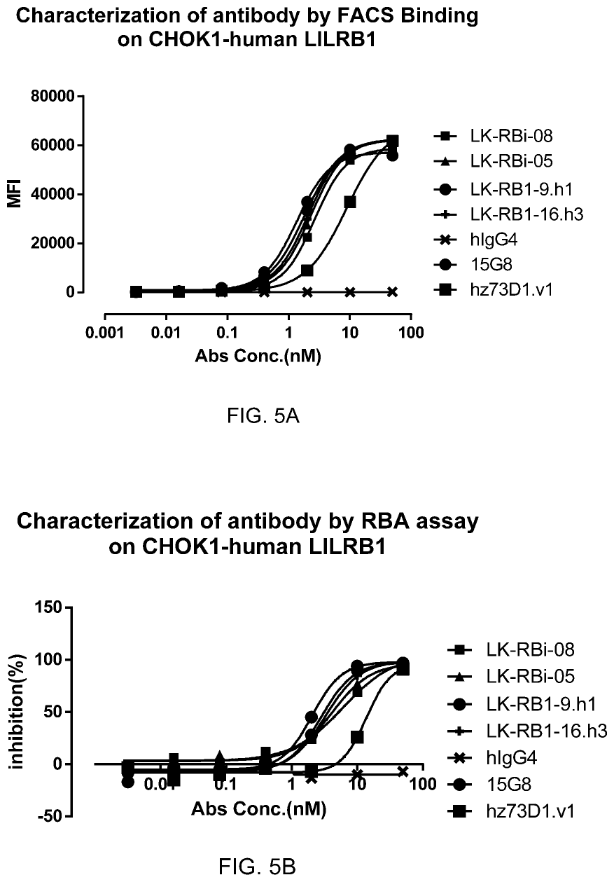

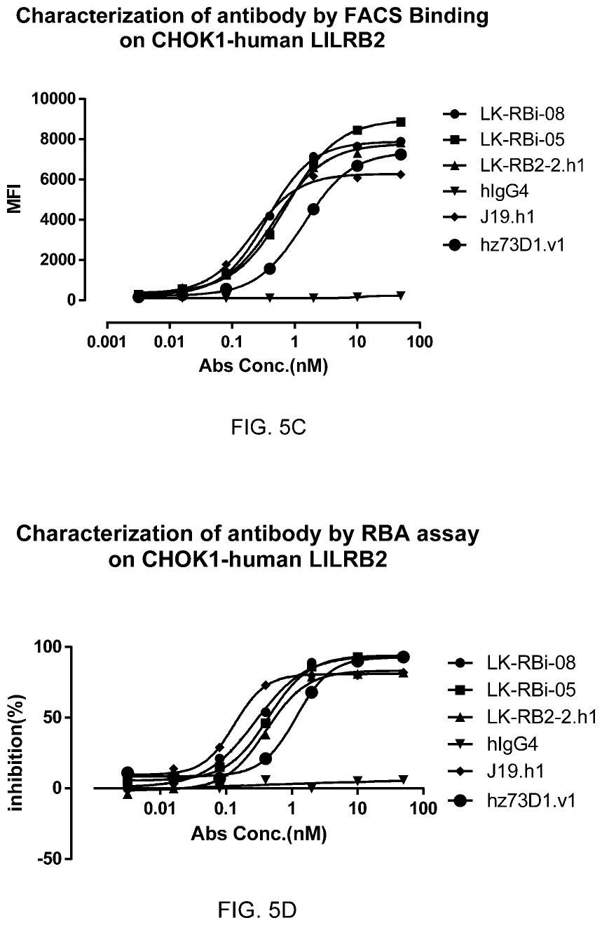

The binding and blocking activity of bispecific antibodies are summarized in Table 18. Binding affinity of bispecific antibodies LK-RBi-05 and LK-RBi-08 against human LILRB1 and LILRB2 was further tested by measuring dissociation rate constant (Kdis) using the Octet system. As shown in Table 19, bispecific antibodies LK-RBi-05 and LK-RBi-08 had good binding affinity against human LILRB1 and LILRB2. Bispecific antibodies LK-RBi-05 and LK-RBi-08 bind to both human LILRB1 and human LILRB2, and blocked the interaction between HLA-G and human LILRB1 or LILRB2 with similar potency comparing to anti-LILRB1 antibody LK-RB1-9.h1, LK-RB1-16.h3 and anti-LILRB2 antibody LK-RB2-2.h1. (FIGS. 5A-5D).

TABLE 18
The binding and blocking activity of bispecific antibodies

| | | | Cell based | Cell based |
|---|---|---|---|---|
| | | | RBA | RBA |
| | CHOK1- | CHOK1- | (CHOK1- | (CHOK1- |
| | hLILRB1 | hLILRB2 | hLILRB1) | hLILRB2) |
| | FACS EC50 | FACS EC50 | EC50 | EC50 |
| Antibody | (nM) | (nM) | (ug/ml) | (ug/ml) |
| LK-RBi-01 | 7.15 | 0.80 | 1.40 | 0.38 |
| LK-RBi-02 | 1.27 | 1.25 | 2.12 | 0.19 |
| LK-RBi-05 | 2.32 | 0.73 | 4.20 | 0.43 |
| LK-RBi-06 | 1.47 | 0.73 | 1.31 | 0.57 |
| LK-RBi-07 | 2.51 | 1.58 | 1.22 | 0.97 |
| LK-RBi-08 | 2.66 | 0.37 | 6.03 | 0.28 |
| LK-RBi-09 | 1.17 | 1.29 | 1.03 | 0.43 |
| LK-RBi-10 | 2.21 | 1.06 | 0.91 | 0.50 |
| LK-RBi-11 | 2.31 | 2.22 | 4.37 | 2.49 |
| hz73D1.v1 | 5.37 | 1.40 | 7.26 | 1.46 |
| 15G8 | 1.17 | Inactive | 2.05 | Inactive |
| J19.h1 | Inactive | 0.27 | Inactive | 0.46 |

TABLE 19
The binding dissociation rate constant (kdis) for
LK-RBi-05 and LK-RBi-08 to
human LILRB1 and human LILRB2

| | Off Rate ranking | Off Rate ranking |
|---|---|---|
| Bispecific | [hLILRB1, | [hLILRB2, |
| antibody | kdis(1/s)] | kdis(1/s)] |
| LK-RBi-05 | 2.10E−04 | 8.24E−06 |
| LK-RBi-08 | 1.66E−04 | 5.68E−05 |

### 7.4 Example 4: Anti-LILRB1 Antibodies Bind to Different Human LILRB1 Variants with Similar Binding Affinity

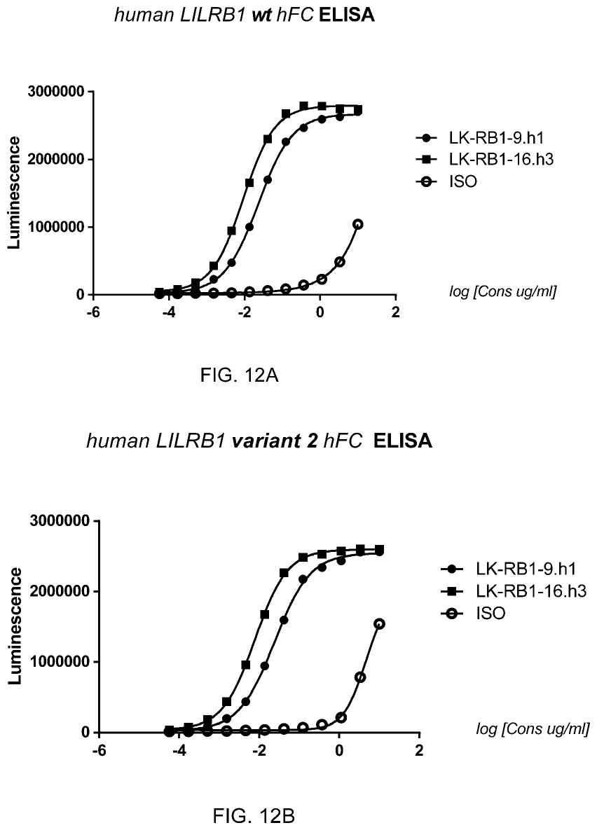

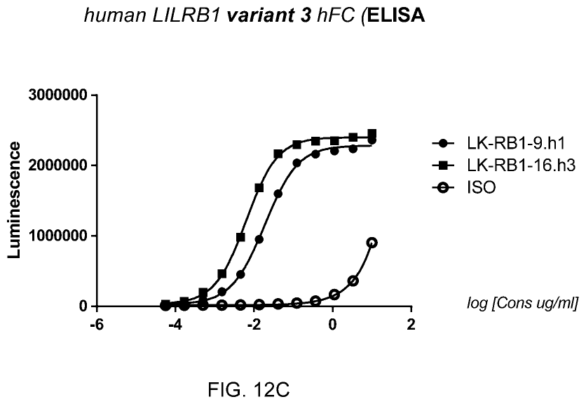

LILRB1 is a highly polymorphic gene. Wild type human LILRB1, Variant 2 and Variant 3 represent three most frequent LILRB1 variants (Liu et al. (2022) Immunogenetics 1-13). Wildtype human LILRB1 is the most frequent variant in European and South Asian populations, with frequencies of 63.6% and 62.6%, respectively. Human LILRB1 Variant 2 is the most frequent variant in East Asian populations (46.2%). Human LILRB1 Variant 3 is enriched in African populations with a frequency of 35.3%. The human LILRB1 Variant 2 and 3 proteins were purchased from Kactusbio, LLC. (Cat: LIL-HM2V1 and LIL-HM2V2). As shown in FIGS. 12A-12C, the antibody LK-RB1-9.h1 and LK-RB1-16.h3 bind to the human LILRB1 wild type (wt), Variant 2 and Variant 3 with similar affinity.

LILRB1 Variant 2 sequence:
(SEQ ID NO: 1204)
MTPILTVLIC LGLSLGPRTH VQAGHLPKPT LWAEPGSVIT
QGSPVTLRCQ GGQETQEYRL YREKKTAPWI TRIPQELVKK
GQFPIPSITW EHTGRYRCYY GSDTAGRSES SDPLELVVTG
AYIKPTLSAQ PSPVVNSGGN VTLQCDSQVA FDGFILCKEG
EDEHPQCLNS QPHARGSSRA IFSVGPVSPS RRWWYRCYAY
DSNSPYEWSL PSDLLELLVL GVSKKPSLSV QPGPIVAPEE
TLTLQCGSDA GYNRFVLYKD GERDFLQLAG AQPQAGLSQA
NFTLGPVSRS YGGQYRCYGA HNLSSEWSAP SDPLDILIAG
QFYDRVSLSV QPGPTVASGE NVTLLCQSQG WMQTFLLTKE
GAADDPWRLR STYQSQKYQA EFPMGPVTSA HAGTYRCYGS
QSSKPYLLTH PSDPLELVVS GPSGGPSSPT TGPTSTSGPE
DQPLTPTGSD PQSGLGRHLG VVIGILVAVI LLLLLLLLLF
LILRHRRQGK HWTSTQRKAD FQHPAGAVGP EPTDRGLQWR
SSPAADAQEE NLYAAVKHTQ PEDGVEMDTR SPHDEDPQAV
TYAEVKHSRP RREMASPPSP LSGEFLDTKD RQAEEDRQMD
TEAAASEAPQ DVTYAQLHSL TLRREATEPP PSQEGPSPAV
PSIYATLAIH
LILRB1 Variant 3 sequence:
(SEQ ID NO: 1205)
MTPILTVLIC LGLSLGPRTH VQAGHLPKPT LWAEPGSVIT
QGSPVTLRCQ GGQETQEYRL YREKKTAPWI TRIPQELVKK
GQFPIPSITW EHAGRYRCYY GSDTAGRSES SDPLELVVTG
AYIKPTLSAQ PSPVVNSGGN VTLQCDSQVA FDGFILCKEG
EDEHPQCLNS QPHARGSSRA IFSVGPVSPS RRWWYRCYAY
DSNSPYEWSL PSDLLELLVL GVSKKPSLSV QPGPIVAPEE
TLTLQCGSDA GYNRFVLYKD GERDFLQLAG AQPQAGLSQA
NFTLGPVSRS YGGQYRCYGA HNLSSEWSAP SDPLDILIAG
QFYDRVSLSV QPGPTVASGE NVTLLCQSQG WMQTFLLTKE
GAADDPWRLR STYQSQKYQA EFPMGPVTSA HAGTYRCYGS
QSSKPYLLTH PSDPLELVVS GPSGGPSSPT TGPTSTSGPE
DQPLTPTGSD PQSGLGRHLG VVIGILVAVI LLLLLLLLLF
LILRHRRQGK HWTSTQRKAD FQHPAGAVGP EPTDRGLQWR
SSPAADAQEE NLYAAVKHTQ PEDGVEMDTR SPHDEDPQAV
TYAEVKHSRP RREMASPPSP LSGEFLDTKD RQAEEDRQMD
TEAAASEAPQ DVTYAQLHSL TLRREATEPP PSQEGPSPAV
PSIYATLAIH

### Functional Characterization of Anti-LILRB1 Antibodies, Anti-LILRB2 Antibodies, and Anti-LILRB1/LILRB2 Bispecific Antibodies.

### 7.5 Example 5: Anti-LILRB1 Antibodies Induced the Activation of Primary Human NK Cells

LILRB1 is expressed on the surface of 30% primary human NK cells. The effect of anti-LILRB1 antibodies on NK cells activation were evaluated in human primary NK cells from healthy human donors. In this experiment, primary NK cells were used as effector cells and K562 cells overexpressing HLA-G were used as target cells. The stable clones of K562 cells expressing human HLA-G (M4T) were produced by Novobio, LLC.

Human HLA-G (M4T) amino acid sequence:
(SEQ ID NO: 1206)
MVVTAPRTLFLLLSGALTLTETWAGSHSMRYFSAAVSRPGRGEPRFIAM
GYVDDTQFVRFDSDSACPRMEPRAPWVEQEGPEYWEEETRNTKAHAQTD
RMNLQTLRGYYNQSEASSHTLQWMIGCDLGSDGRLLRGYEQYAYDGKDY
LALNEDLRSWTAADTAAQISKRKCEAANVAEQRRAYLEGTCVEWLHRYL
ENGKEMLQRADPPKTHVTHHPVFDYEATLRCWALGFYPAEIILTWQRDG
EDQTQDVELVETRPAGDGTFQKWAAVVVPSGEEQRYTCHVQHEGLPEPL
MLRWKQSSLPTIPIMGIVAGLVVLAAVVTGAAVAAVLWRKKSSD

Briefly, the co-culture was treated with anti-LILRB1 antibodies for 4 hours at 37° C. and NK cell activation was analyzed by measuring activation marker CD107a levels on NK cells. CD107a FACS assay was carried out in 96 well U shape plates. The staining primary antibody mixture [Pacific Blue™ anti-human CD45, BioLegend, 304029; PE anti-human CD107a (LAMP-1), BioLegend, 328608; CD85j (LILRB1) Monoclonal Antibody (HP-F1), APC, eBioscience, 17-5129-42; PerCP/Cy5.5 anti-human CD137 (4-1BB), BD Pharmingen, 309814] was added to the plate. After 30 min incubation at 4° C., the plates were analyzed by FACS.

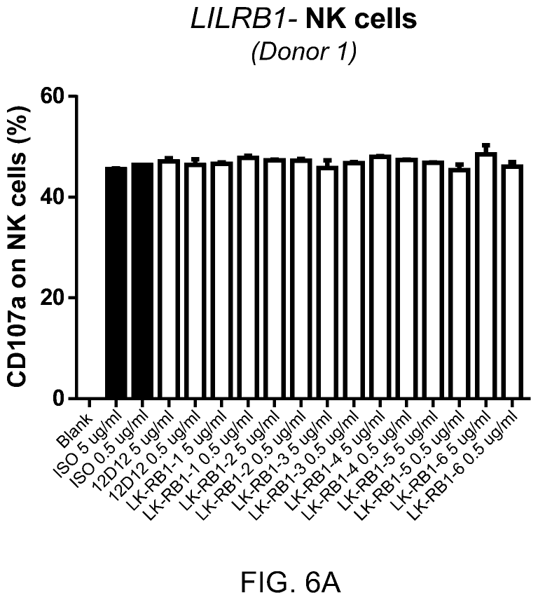

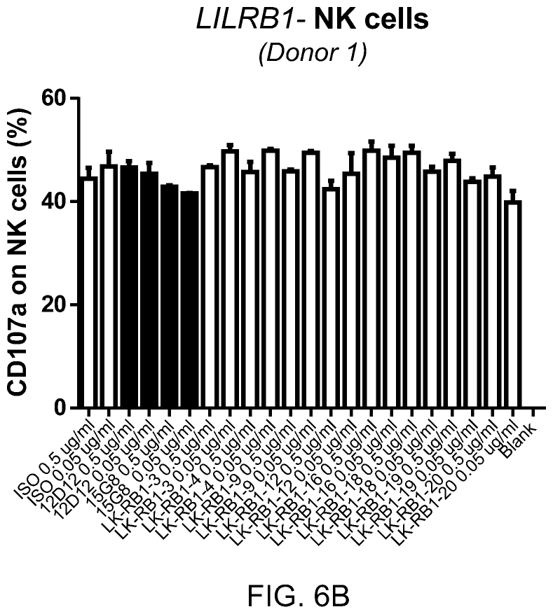

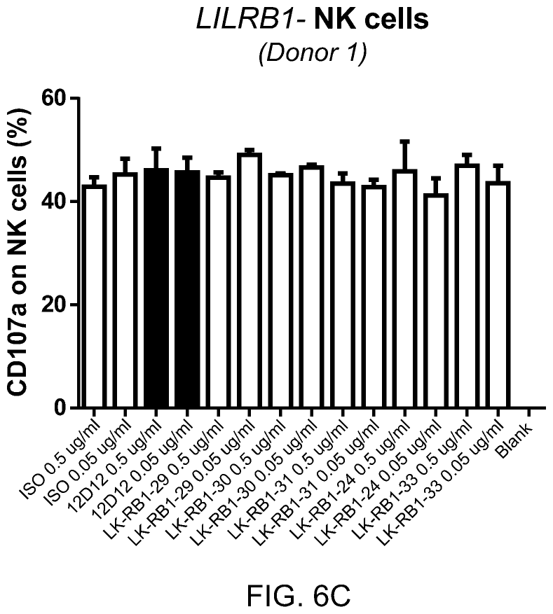

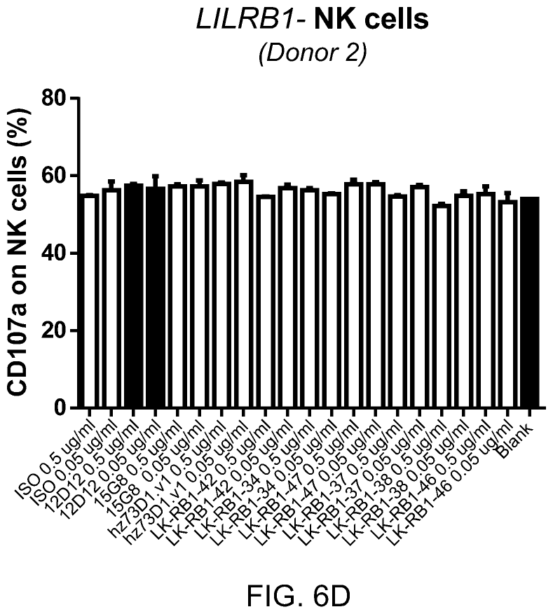

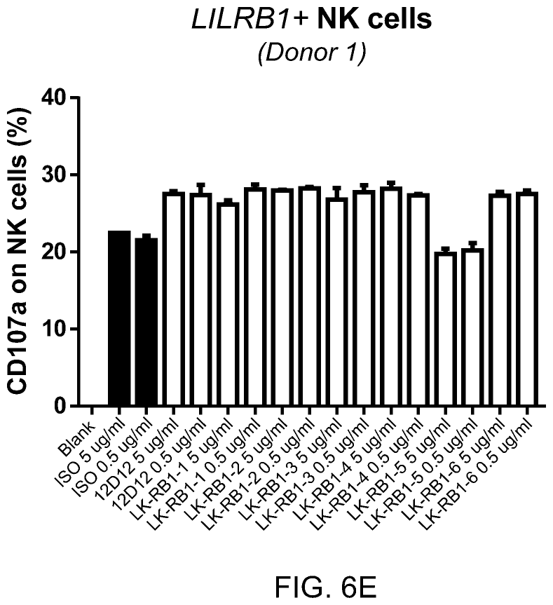

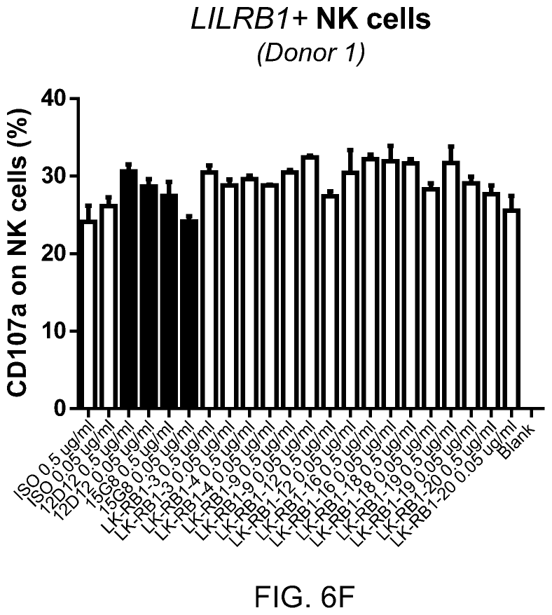

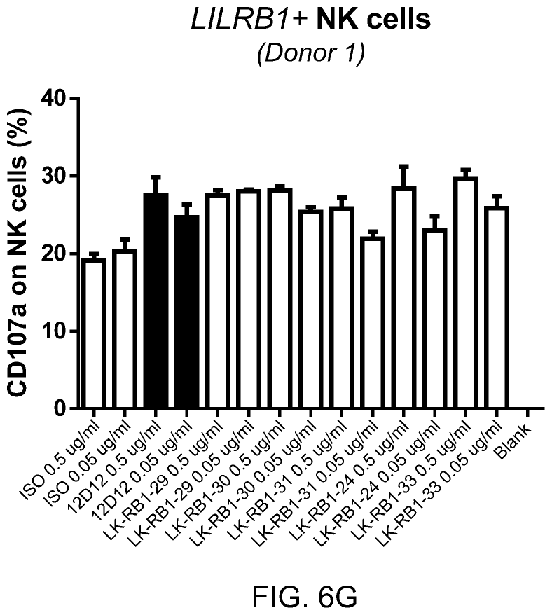

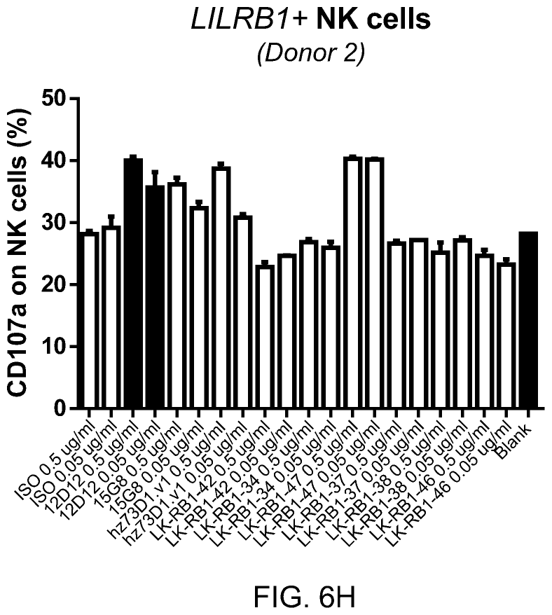

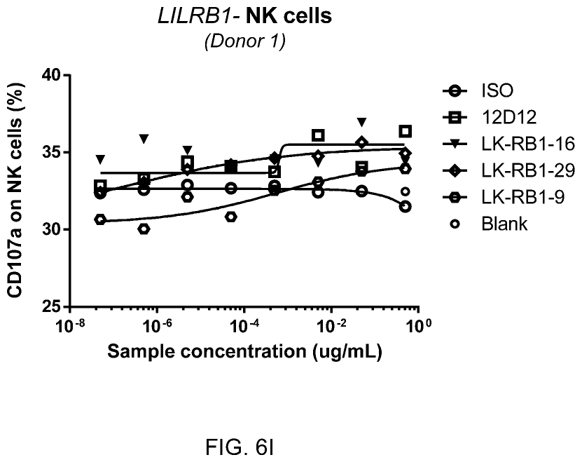

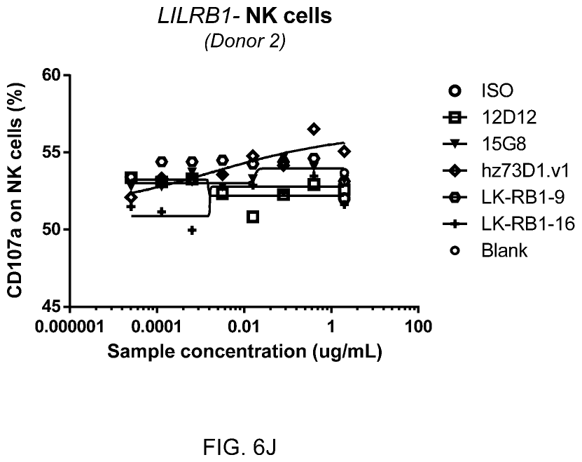

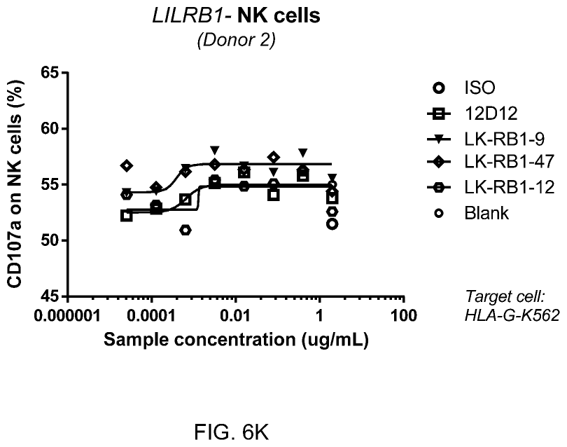

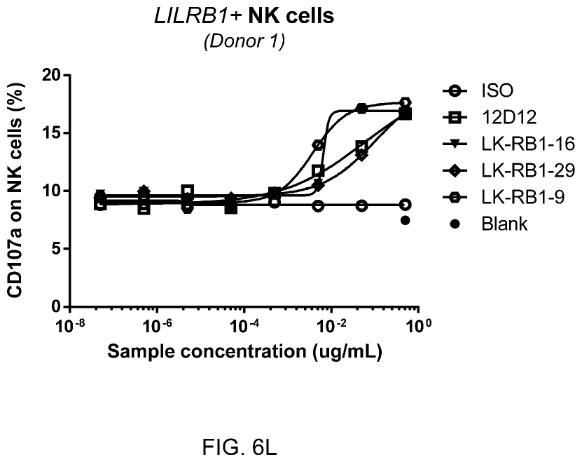

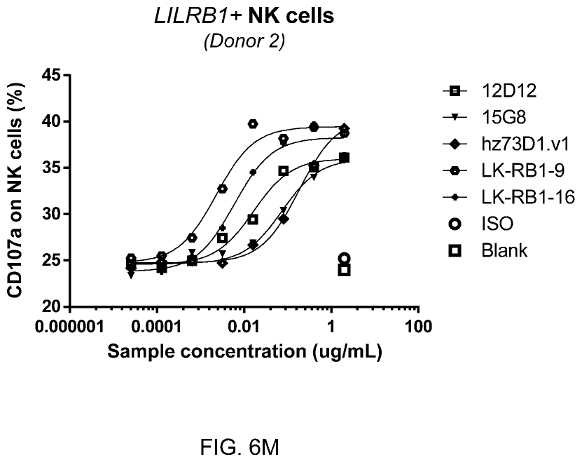

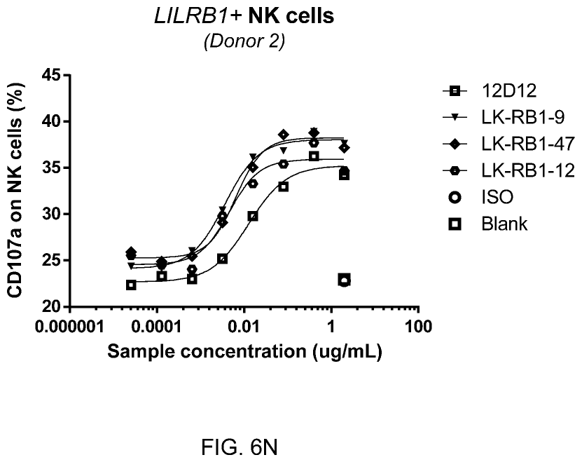

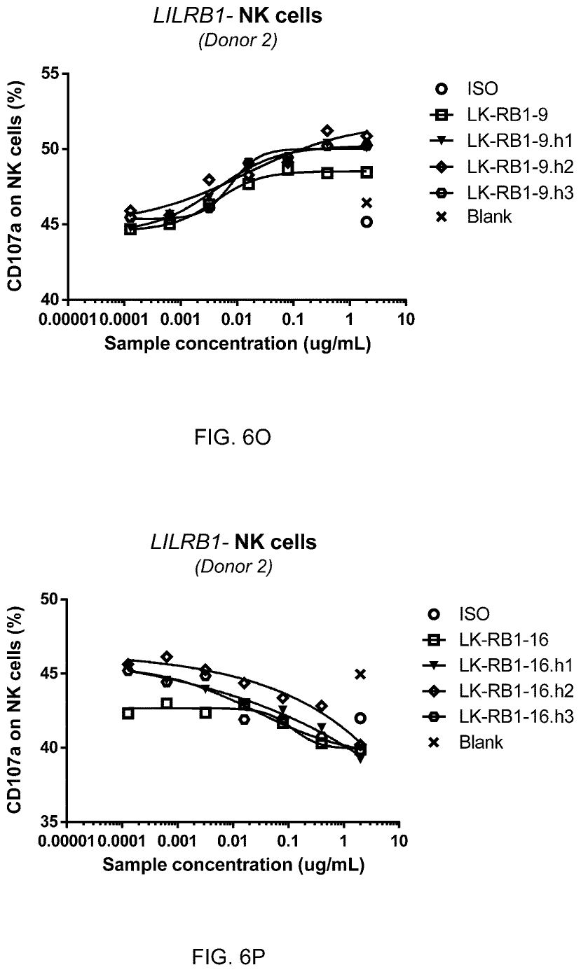

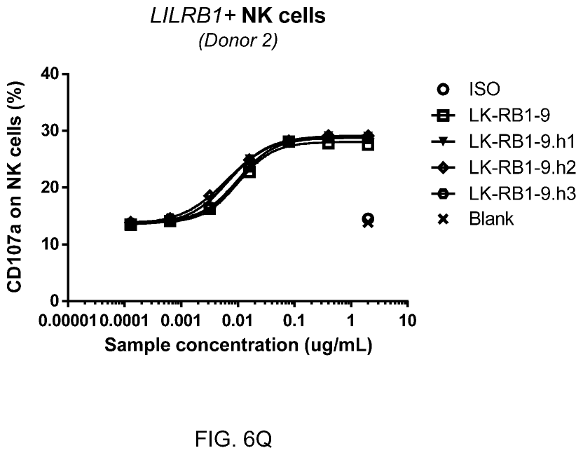

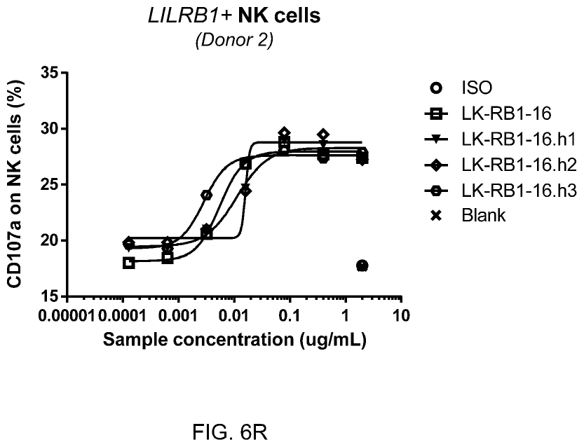

As shown in FIGS. 6A-6H, anti-LILRB1 antibodies increased % CD107a positive cells in LILRB1 positive NK cells, but not in LILRB1 negative NK cells. Dose response study further confirmed that anti-LILRB1 antibodies potently induced LILRB1 positive NK cell activation as measured by CD107a marker (FIGS. 61-6N). Humanized antibodies LK-RB1-9.h1, LK-RB1-9.h2, LK-RB1-9.h3, LK-RB1-16.h1, LK-RB1-16.h2 and LK-RB1-16.h3 had similar activity on NK cell activation comparing to the parental clone LK-RB1-9 and LK-RB1-16 (FIGS. 6O-6R).

### 7.6 Example 6: Anti-LILRB1 Antibodies Enhanced Macrophage Phagocytosis

Anti-LILRB1 antibodies were evaluated for the ability to enhance macrophage phagocytosis of tumor cells. In this experiment, monocyte derived macrophages were used as effector cells and Raji cells (HLA-A2 positive) were used as target cells. Effector cells and target cells were co-cultured at a 1:1 ratio.

Briefly, human monocyte derived macrophages were obtained from human peripheral blood mononuclear cell (PBMC). Human PBMC was cultured in RPMI 1640 medium containing 10% FBS (heat inactivated) and M-CSF (40 ng/ml). After 10 days of culture, monocyte derived macrophages were collected and labeled with 2 μM Dye eFluor™ 670. Then, the labeled macrophages were seeded at 100 μL/well into flat bottom cell culture plate and cultured overnight. Raji cells were labeled with CSFE and then seeded onto 96 well plate containing macrophages (4×104 cells/well). Antibody Hu5F9-G4 (anti-CD47 antibody) and anti-LILRB1 antibodies were added to the plate according to the plate map. The co-culture was cultured for additional 4 hours. The cells from the co-culture were collected and analyzed by flow cytometry immediately (FITC/APC channel).

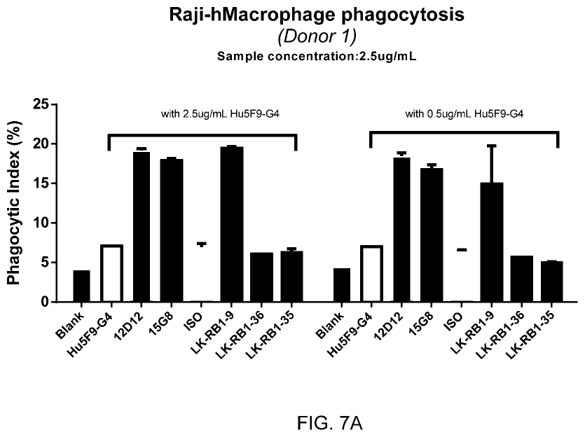

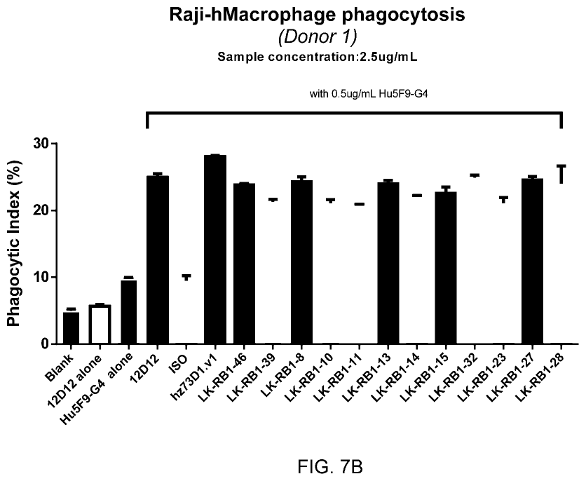

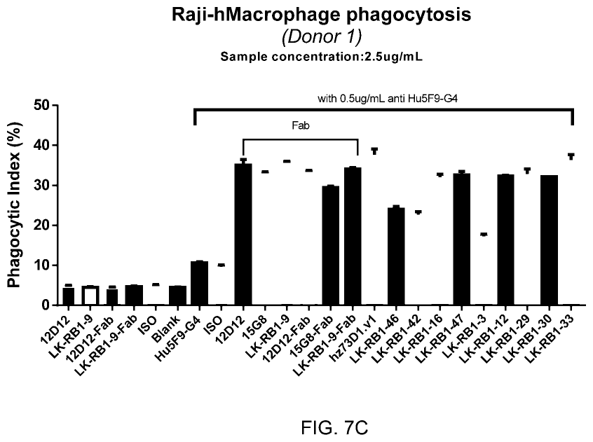

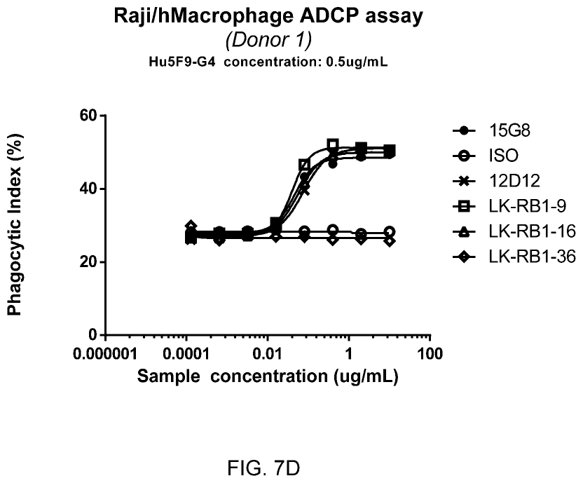

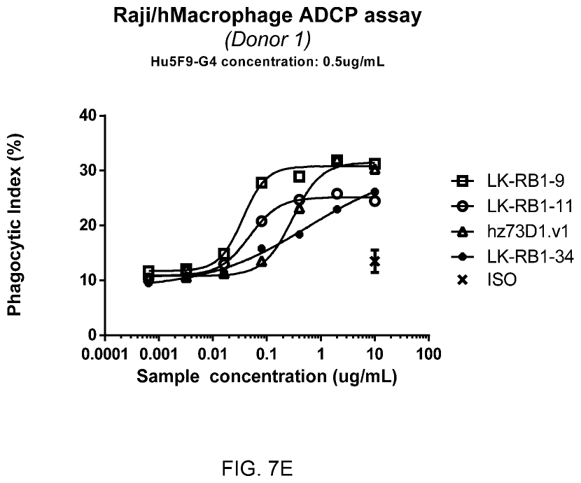

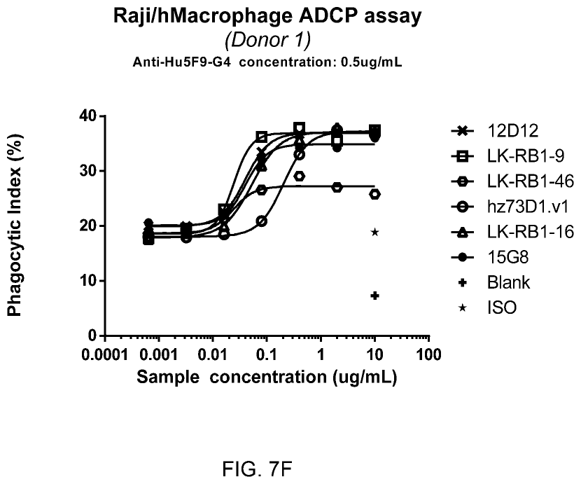

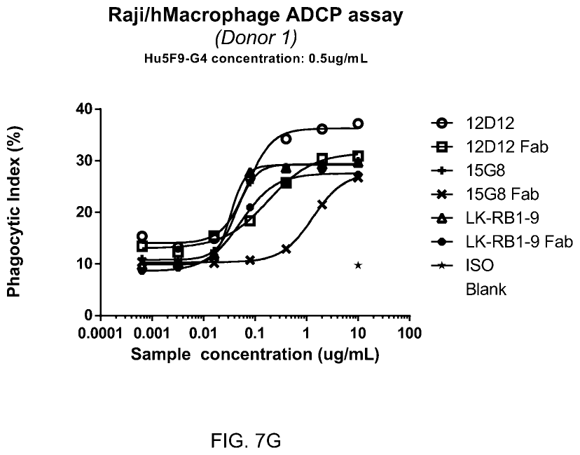

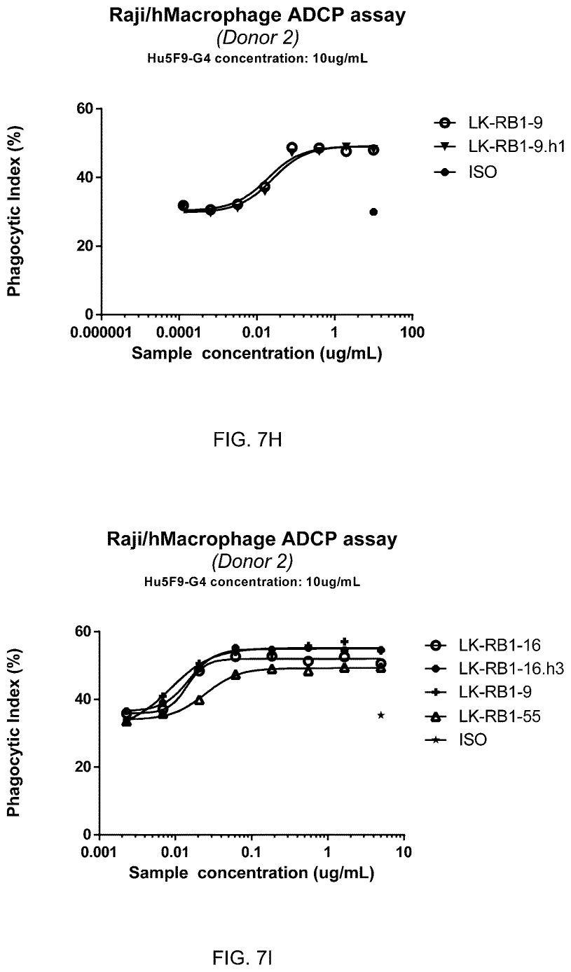

Anti-LILRB1 antibodies further enhanced Hu5F9-G4 (anti-CD47)-induced phagocytosis (FIGS. 7A-7B). The effect of anti-LILRB1 antibody on phagocytosis was not dependent on antibody Fc region, as Fab version of anti-LILRB1 antibodies could also increase the phagocytosis in the presence of Hu5F9-G4 (FIG. 7C). Both Fab and mAb version of anti-LILRB1 antibodies could dose dependently increase phagocytosis in the presence of Hu5F9-G4 (FIGS. 7D-7G). LK-RB1-34, LK-RB1-36 and LK-RB1-46 were clones with no or weak blocking activity against the interaction between LILRB1 and HLA-G. As shown in FIGS. 7D-7F, these clones had no or very weak activity, suggesting blocking activity of anti-LILRB1 antibody was required to promote phagocytosis. Humanization generally did not affect the activity on phagocytosis, as the potency of LK-RB1-9.h1 and LK-RB1-16.h3 were comparable to the parental clone LK-RB1-9 and LK-RB1-16 in phagocytosis assay (FIGS. 7H-7I).

### 7.7 Example 7: Anti-LILRB1 Antibodies Increased the Activity of CD8+ T Cells

LILRB1 is expressed on the surface of 5-20% primary human CD8+ T cells. Anti-LILRB1 antibodies were evaluated for the ability to increase CD8+ T cells activity. In this experiment, primary CD8+ T cells were used as the effector cells and CHOK1 cells overexpressing HLA-G/PDL-1/OKT3 (generated by Celetrix. LLC) were used as the target cells. Effector cells and target cells were co-cultured at a ratio of 5:1.

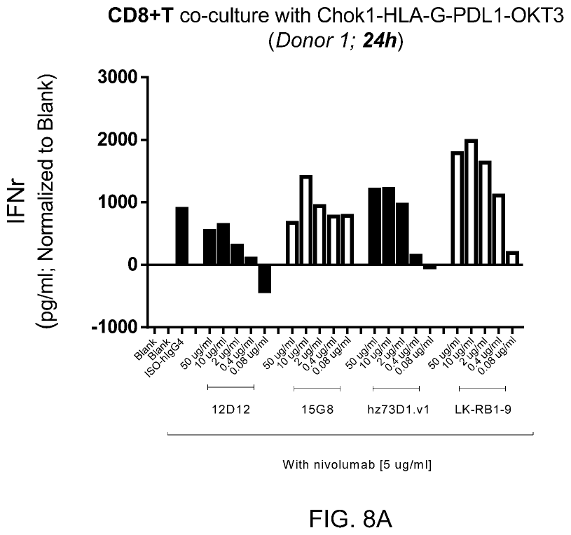

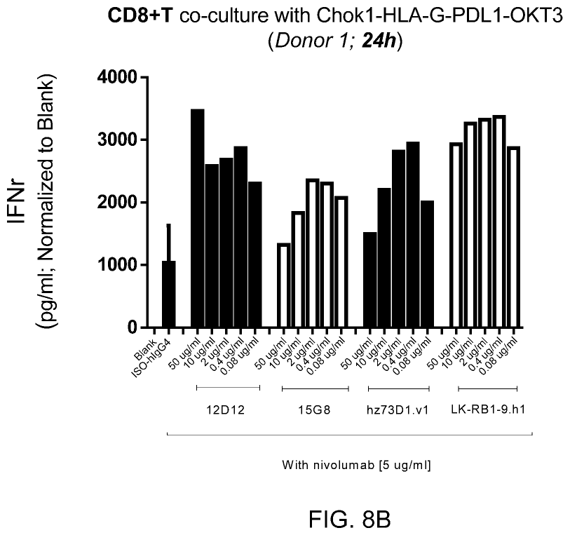

The effect of the anti-LILRB1 antibodies on CD8+ T cells activation was determined by measuring IFNγ level in the culture medium. Briefly, CHOK1-HLA-G/PDL-1/OKT3 cells (4×105 cells/mL) was seeded into 96 well culture plate and treated with 10 μg/ml anti-PD-1 antibody (Nivolumab). Anti-LILBR1 antibodies were pre-incubated with primary CD8+ T cells (2×106 cells/mL) for 30 minutes at 37° C. Then anti-LILBR1 antibodies and effector cells were added to plate containing target cells and Nivolumab. After 24 h incubation at 37° C., the IFNγ level in the culture medium was measured by ELISA (Human IFN-γ ELISA MAX™ Standard, BioLegend, 430101). As shown in FIGS. 8A-8B, antibody LK-RB1-9 and LK-RB1-9.h1 significantly activated CD8+ T cells as measured by IFNγ levels in the culture medium. The effect was comparable or stronger than the reference antibodies 12D12, 15G8 and hz73D1.v1.

### 7.8 Example 8: Anti-LILRB1 and Anti-LILRB2 Antibodies Enhanced LPS-Induced TNFα Release in Human Primary Macrophages

LILRB1 and LILRB2 are immune inhibitory receptor on macrophage. The effect of anti-LILRB1 and anti-LILRB2 antibodies on macrophage activation was studied using monocyte derived macrophages (LILRB1 and LILRB2 positive, HLA-ABC positive). Briefly, human monocyte derived macrophages were obtained from human PBMC. Human PBMC was cultured with RPMI 1640 medium containing 10% FBS (heat inactivated) and M-CSF (50 ng/ml) for 10 days. Macrophages were collected, seeded (2×10⁵ cells/well) and treated with antibodies in the presence of Lipopolysaccharide (LPS) (50 ng/ml). After 24 h incubation at 37° C., TNFα and IL-10 levels in the culture medium were measured by ELISA (Human TNF alpha ELISA Kit, Abcam, ab181421; Human IL-10 ELISA Kit, Abcam, ab185986).

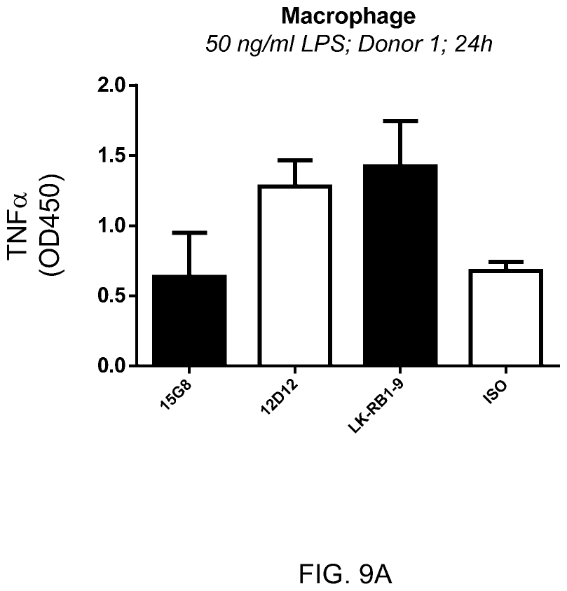

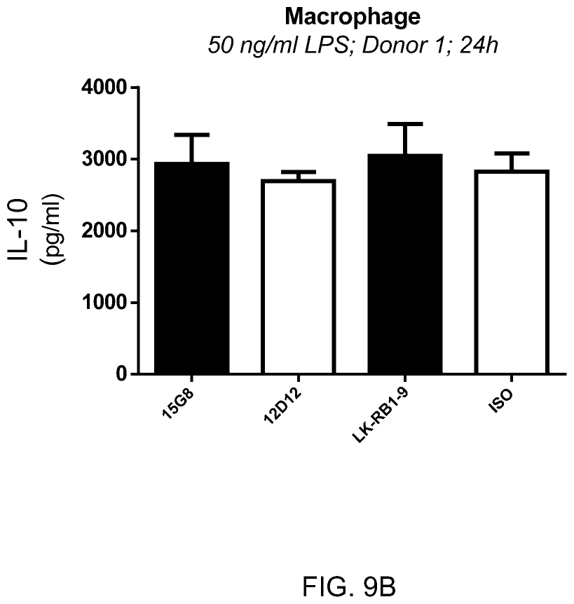

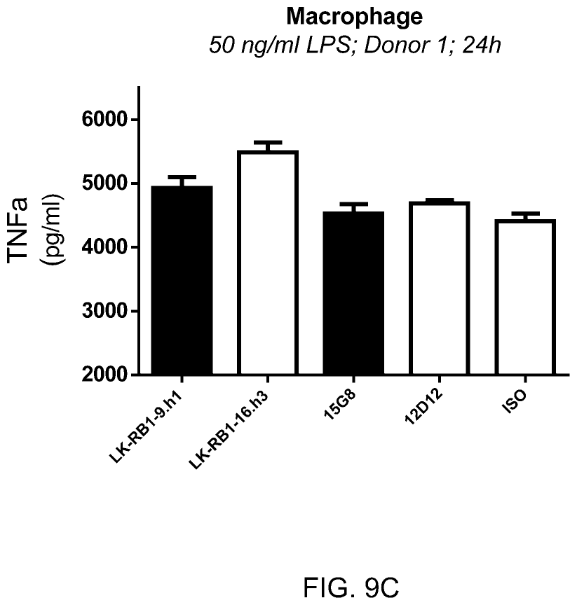

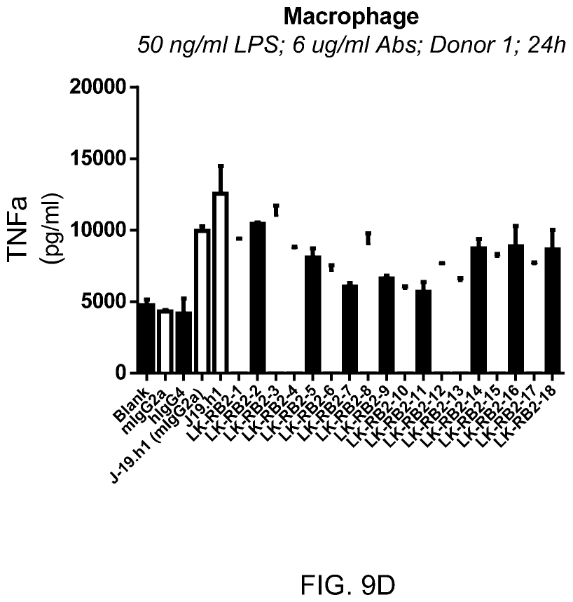

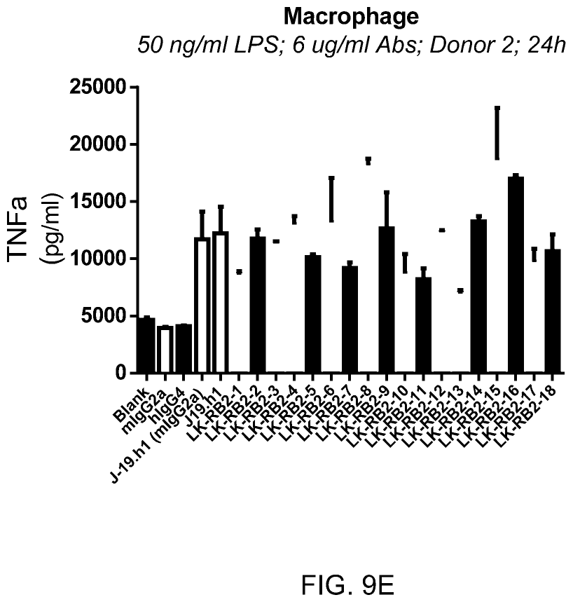

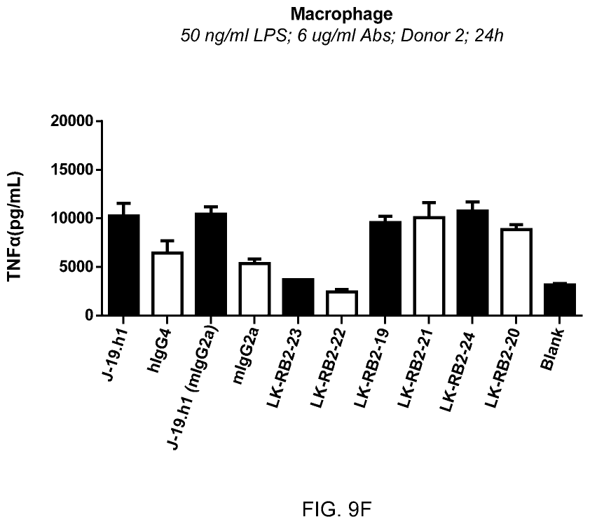

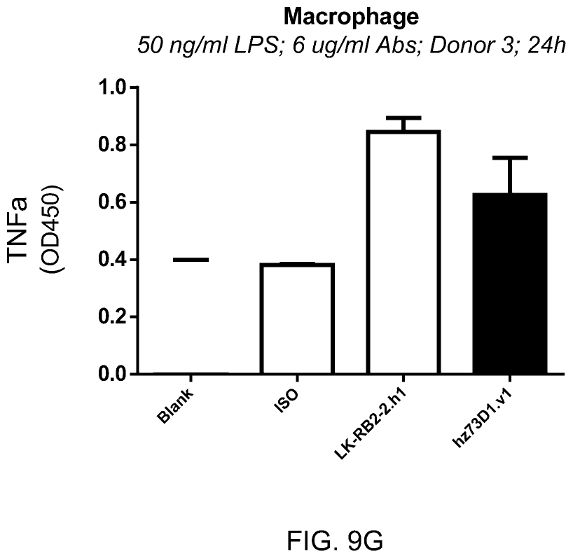

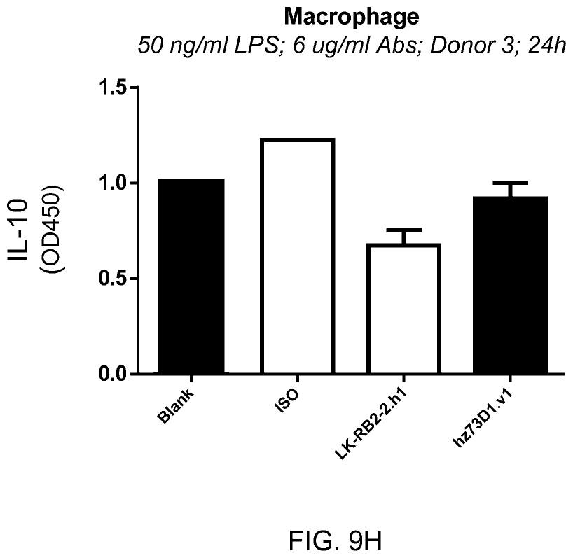

As shown in FIG. 9A, LK-RB1-9 and 12D12, but not 15G8, further increased LPS-induced TNFα release. They had no effect on IL10 release in the presence of LPS (FIG. 9B). Humanized antibody LK-RB1-9.h1 and LK-RB1-16.h3 both significantly increased LPS-induced TNFα release in primary macrophage (FIG. 9C). Anti-LILRB2 antibodies increased LPS-induced TNFα release in primary macrophage (FIGS. 9D-9F). Humanized antibody LK-RB2-2.h1 increased LPS-induced TNFα release and reduced LPS-induced IL-10 release in primary macrophage (FIGS. 9G-9H).

### 7.9 Example 9: Anti-LILRB2 Antibodies Enhanced LPS-Induced TNFα Release in Human Primary PBMC

LILRB2 acts as an immune suppressor to inhibit myeloid cells activation. The effect of anti-LILRB2 antibody on myeloid cells activation was studied using human PBMC (LILRB2 positive, HLA-ABC positive). Briefly, hPBMC were seeded (2×105 cells/well) and treated with anti-LILRB2 antibodies in the presence of lipopolysaccharide (LPS) (50 ng/ml). After 24 h incubation at 37° C., TNFα levels in the culture medium were measured by ELISA (Human TNF-alpha DuoSet ELISA, R&D Systems, DY210-05).

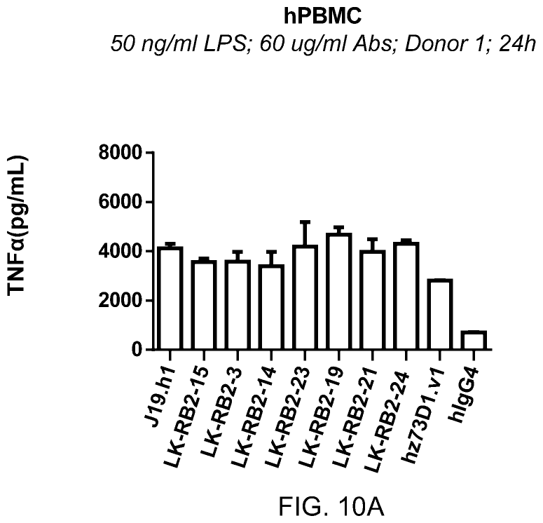

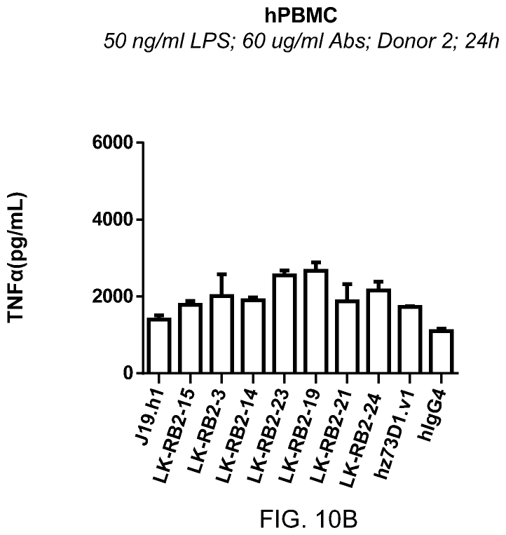

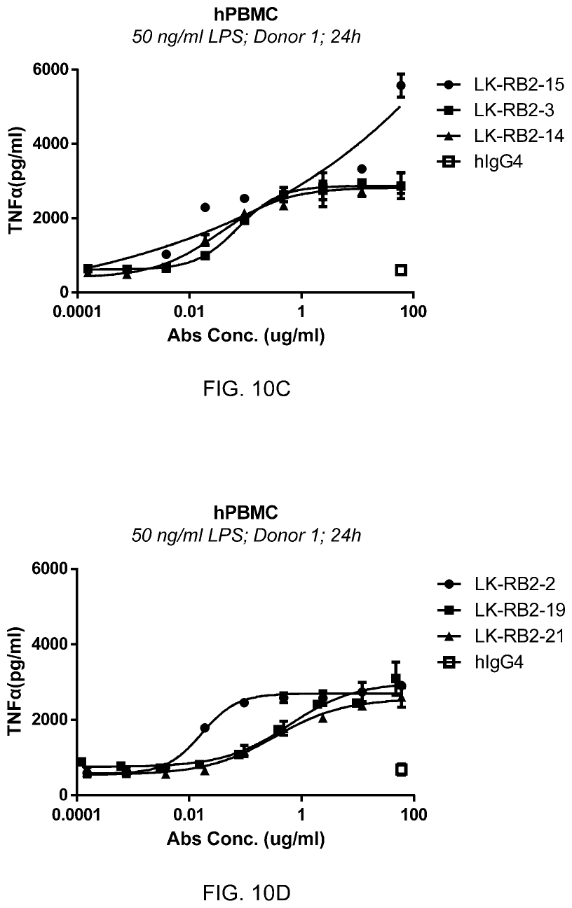

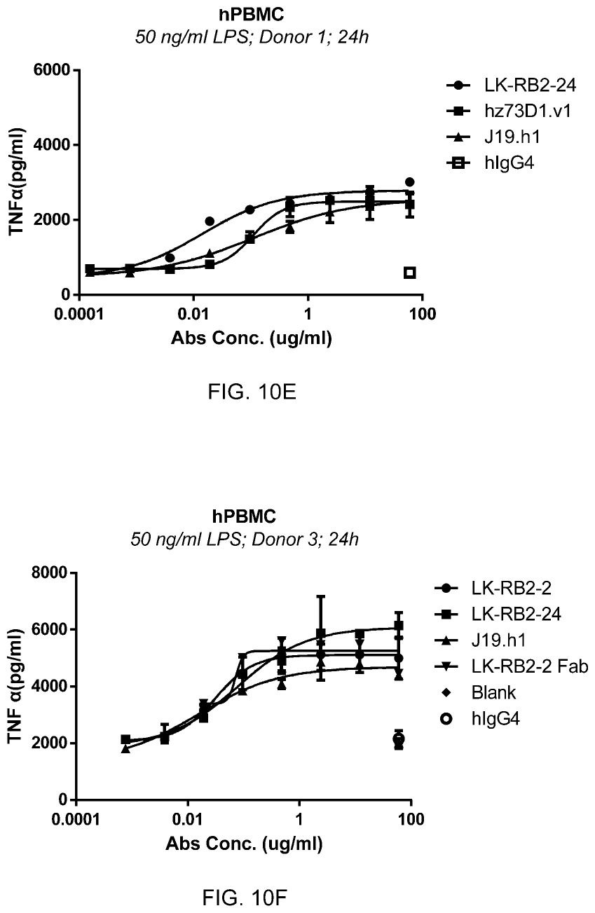

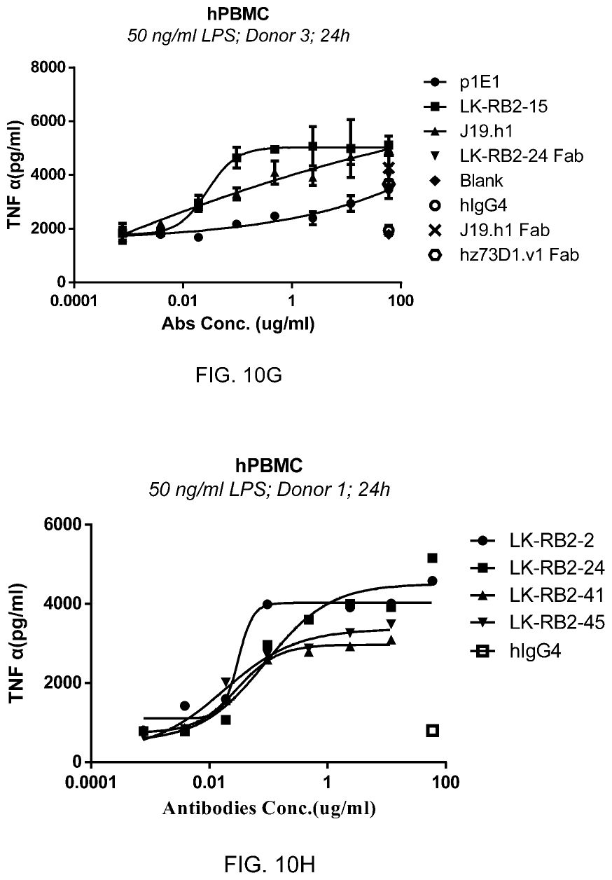

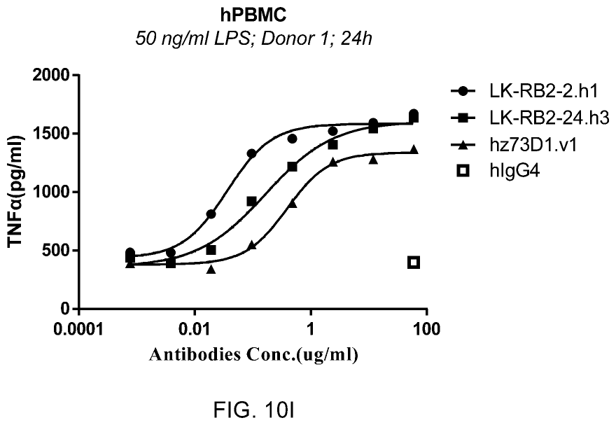

As shown in FIGS. 10A and 10B, anti-LILRB2 antibodies increased LPS-induced TNFα release in human PBMC. Both Fab and mAb version of anti-LILRB2 antibodies could dose dependently increase LPS-induced TNFα release in human PBMC (FIGS. 10C-10H). Humanized antibody LK-RB2-2.h1 and LK-RB2-24.h3 increased LPS-induced TNFα release in human PBMC (FIG. 101).

### 7.10 Example 10: Anti-LILRB2 Antibodies Enhanced Anti-CD3 Induced TNFα and IFN-γ Release in Human Primary PBMC

To assess whether anti-LILRB2 antibody treatment could increase cytokine release in the presence of T cell stimulus, human primary PBMC were stimulated with anti-CD3 antibody (OKT3, BioLegend, 317325) to induce T cell activation. Briefly, hPBMC were seeded (2.5×105 cells/well) to OKT3 antibody (0.2 μg/ml) coated plate and treated with anti-LILRB2 antibodies. After 48h incubation at 37° C., TNFα and IFN-γ levels in the culture medium were measured by ELISA (Human TNF alpha ELISA Kit, Abcam, ab181421; Human IFN-γ ELISA MAX™ Standard, BioLegend, 430101).

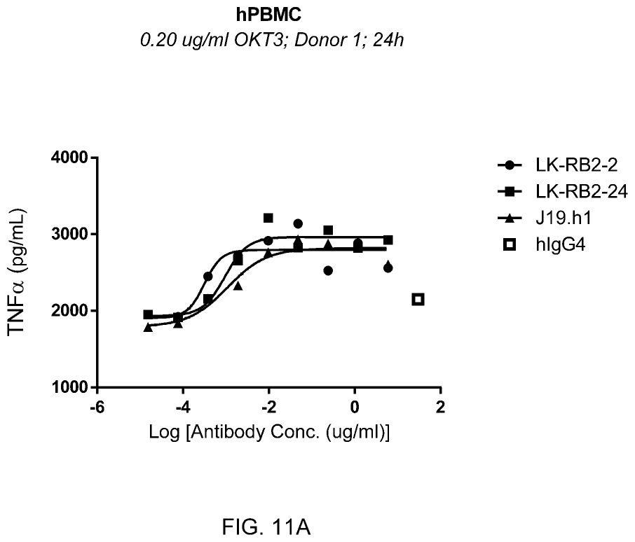

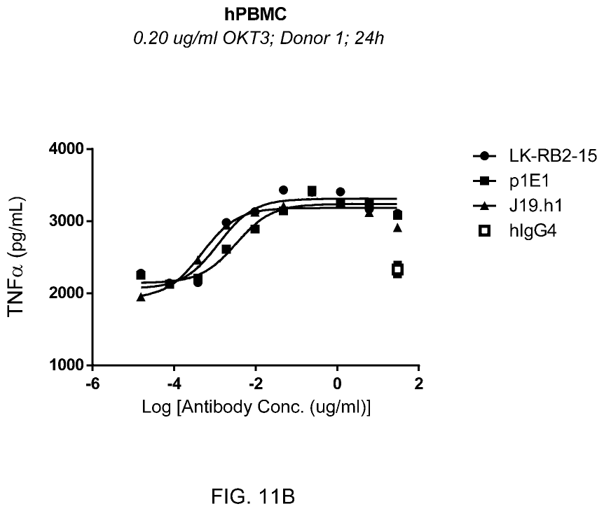

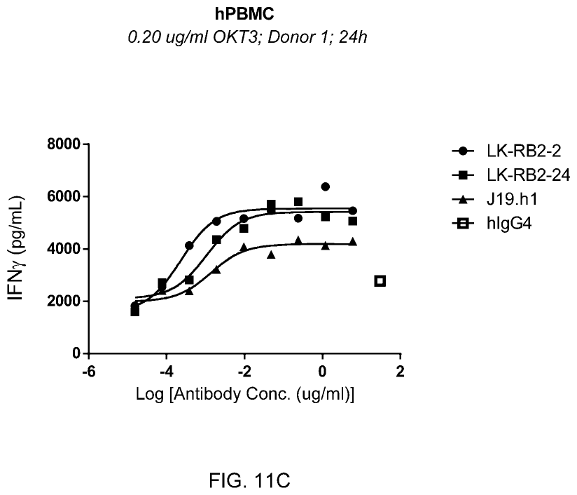

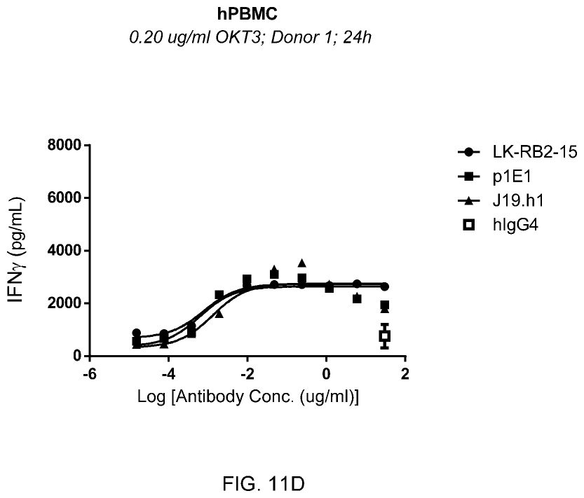

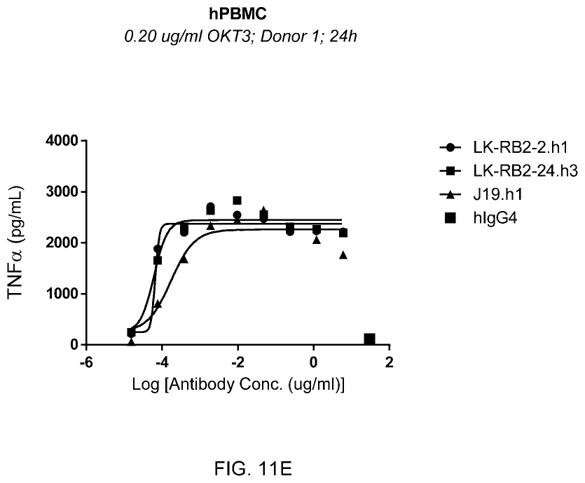

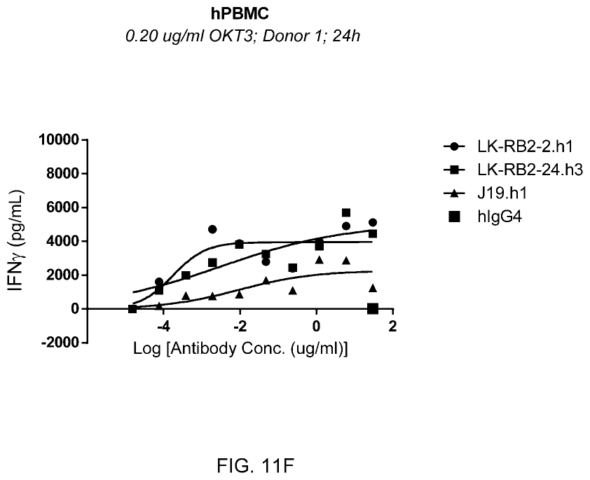

As shown in FIGS. 11A-11D, anti-LILRB2 antibodies could dose dependently increase OKT3-induced TNFα and IFN-γ release in human primary PBMC. Humanized antibody LK-RB2-2.h1 and LK-RB2-24.h3 increased OKT3-induced TNFα and IFN-γ release in human PBMC (FIGS. 11E and 11F).

### 7.11 Example 11: Anti-LILRB1/LILRB2 Bispecific Antibodies Induced the Activation of Primary Human NK Cells

To assess whether anti-LILRB1/LILRB2 bispecific antibodies treatment could also increase % CD107a positive cells in LILRB1 positive NK cells, 9 bispecific antibodies were evaluated in primary human NK cells activation assay.

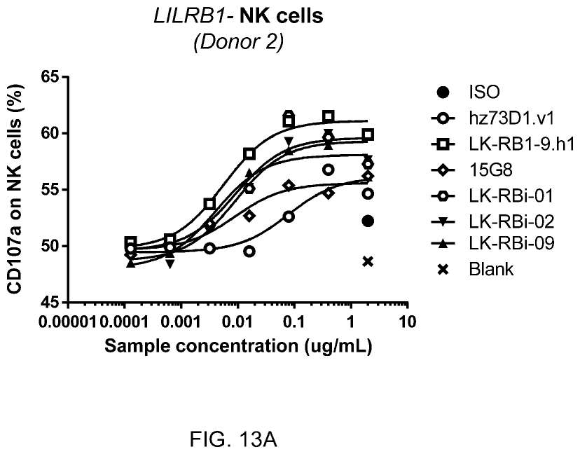

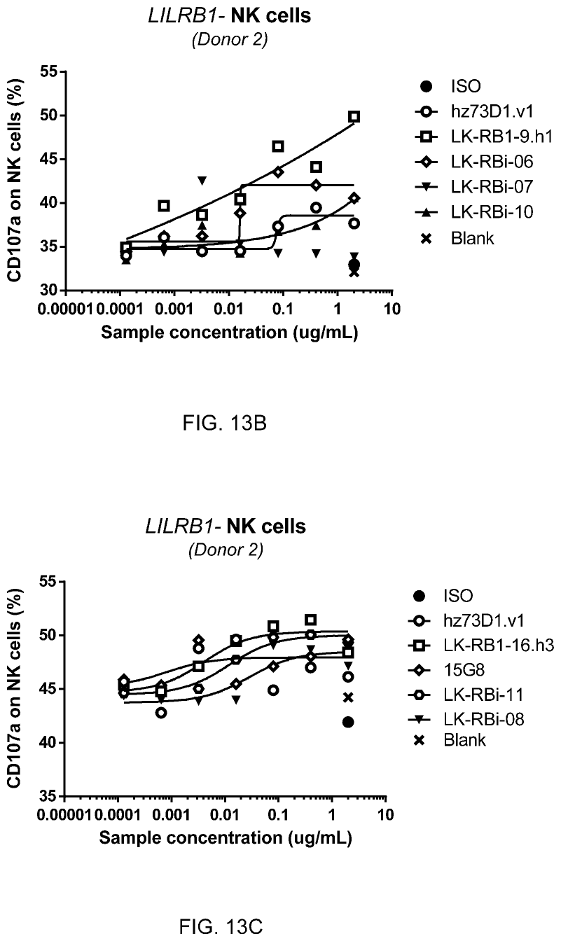

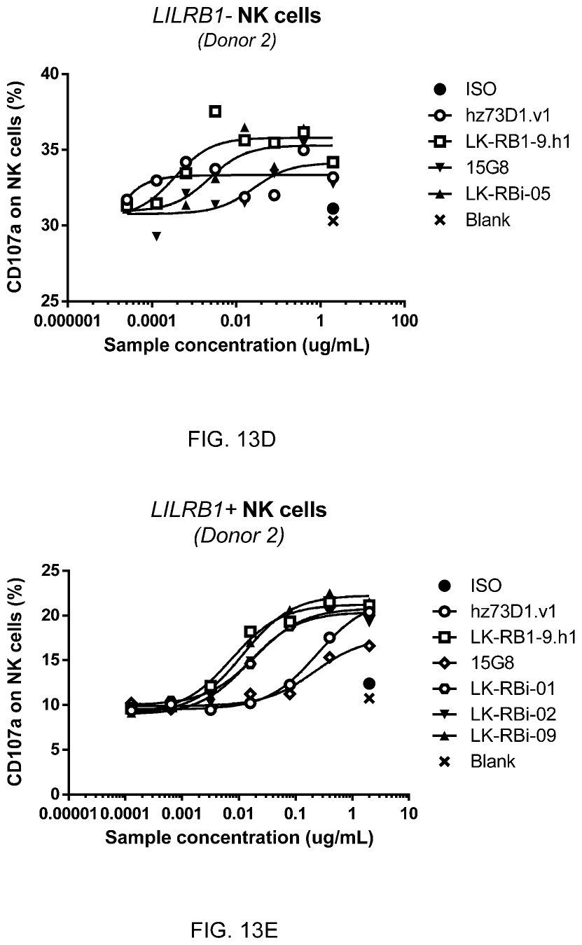

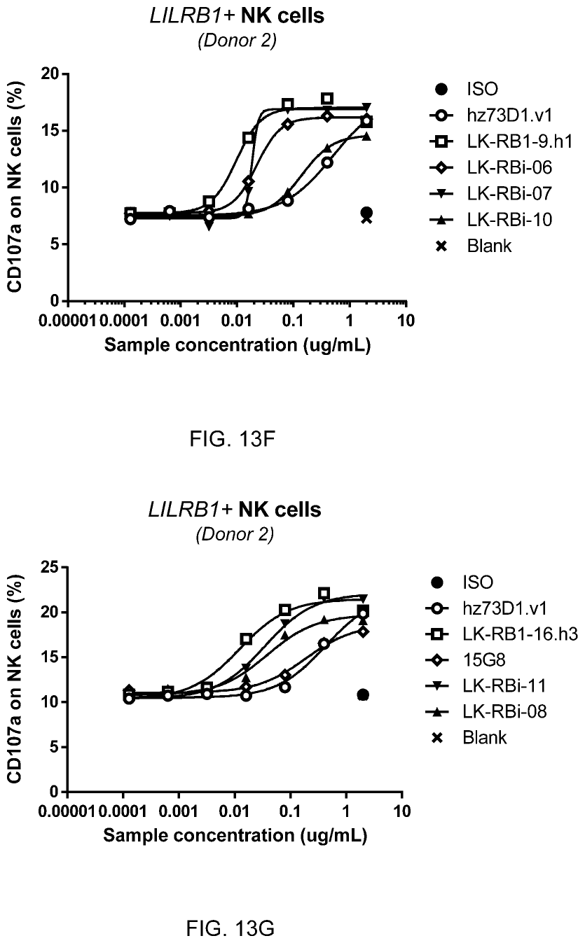

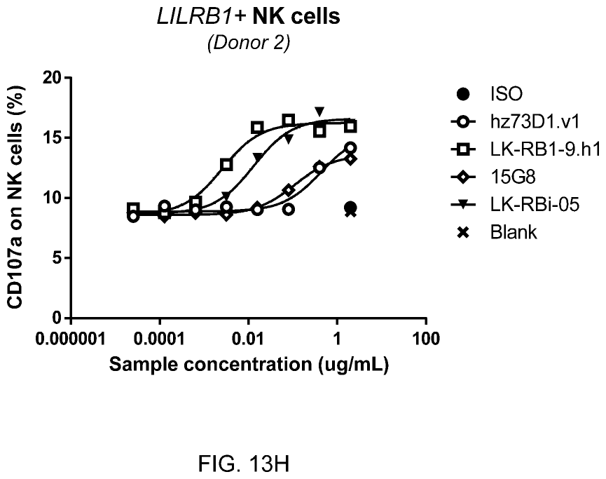

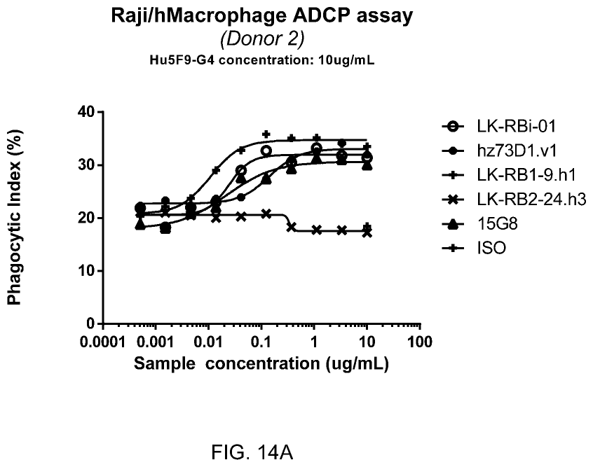

As shown in FIGS. 13A-13H, bispecific antibodies dose dependently increased % CD107a positive cells in LILRB1 positive NK cells. Bispecific antibodies design generally did not affect the activity on NK activation, LK-RBi-01, LK-RBi-02, LK-RBi-05, LK-RBi-06, LK-RBi-07, LK-RBi-08, LK-RBi-09 and LK-RBi-11 had similar activity on LILRB1 positive NK cell activation comparing to anti-LILRB1 antibody LK-RB1-9.h1 and LK-RB1-16.h3 (FIGS. 13E-13H).

### 7.12 Example 12: Anti-LILRB1/LILRB2 Bispecific Antibodies Enhanced Macrophage Phagocytosis

To assess whether anti-LILRB1/LILRB2 bispecific antibodies treatment could also increase the phagocytosis in the presence of Hu5F9-G4, bispecific antibodies were evaluated in phagocytosis assay.

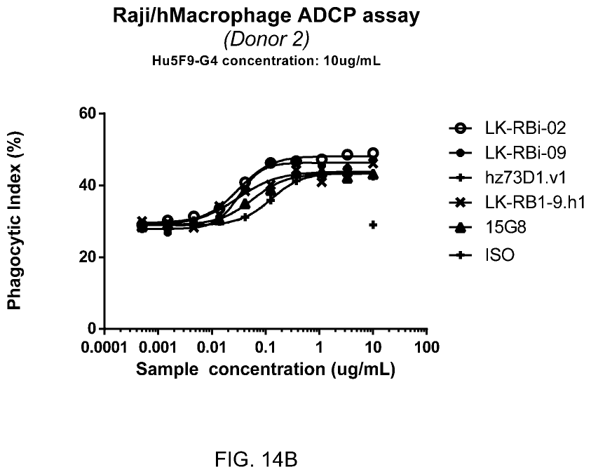

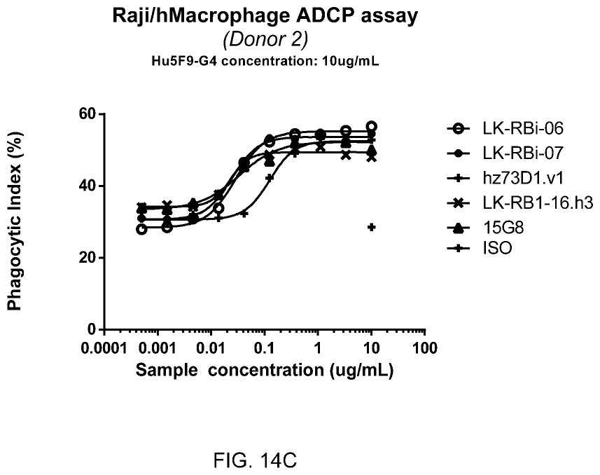

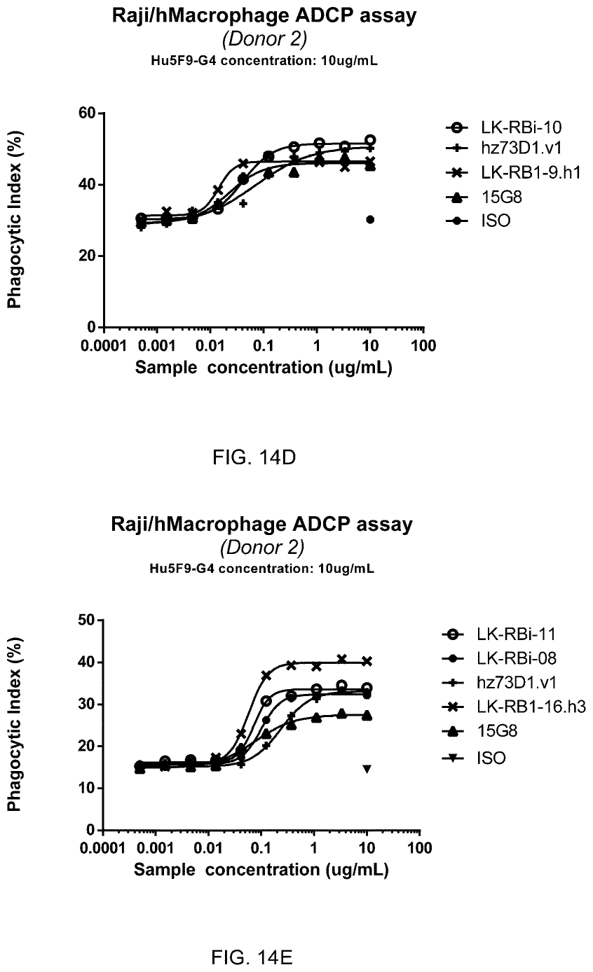

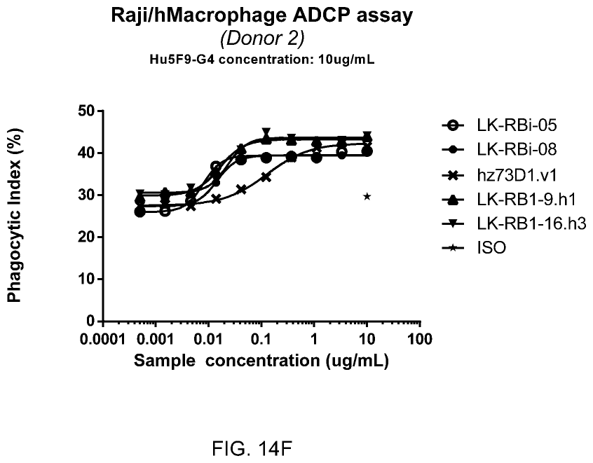

As shown in FIGS. 14A-14F, bispecific antibodies dose dependently increased phagocytosis in the presence of Hu5F9-G4. Bispecific antibodies design generally did not affect the activity on phagocytosis, LK-RBi-01, LK-RBi-02, LK-RBi-05, LK-RBi-06, LK-RBi-07, LK-RBi-08, LK-RBi-09, LK-RBi-10 and LK-RBi-11 had similar activity to the parental clone LK-RB1-9.h1 and LK-RB1-16.h3.

### 7.13 Example 13: Anti-LILRB1/LILRB2 Bispecific Antibodies Increased CD8+ T Cells Activity

To assess whether anti-LILRB1/LILRB2 bispecific antibodies treatment could activate CD8+ T cells as measured by IFNγ levels in the culture medium, 2 bispecific antibodies were evaluated in CD8+ T cells activation assay.

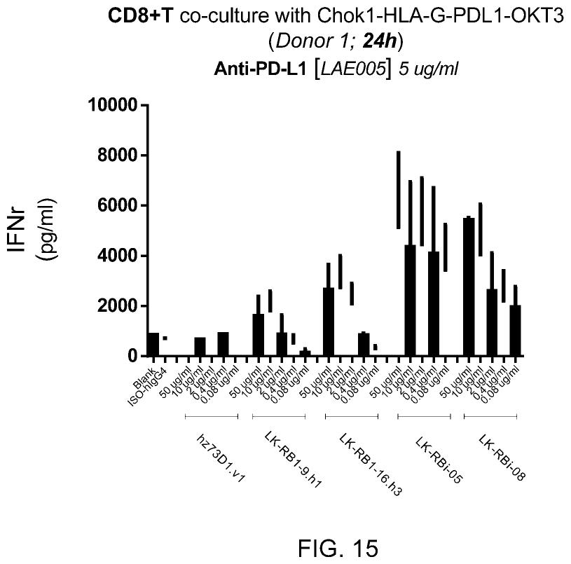

As shown in FIG. 15, in the presence of anti-PD-L1 antibody (LAE005), bispecific antibody LK-RBi-05 and LK-RBi-08 significantly increased IFNr levels in the culture medium. This T cell activating effect of bispecific antibodies (LK-RBi-05, LK-RBi-08) was stronger than anti-LILRB1 antibody (LK-RB1-9.h1, LK-RB1-16.h3) and hz73D1.v1.

### 7.14 Example 14: Anti-LILRB1/LILRB2 Bispecific Antibodies Enhanced LPS-Induced TNFα Release in Human Primary Macrophages

To assess whether anti-LILRB1/LILRB2 bispecific antibodies treatment could increase LPS-induced TNFα release in human primary macrophage, bispecific antibodies and anti-LILRB1 antibodies and anti-LILRB2 antibody were evaluated in macrophage activation assay.

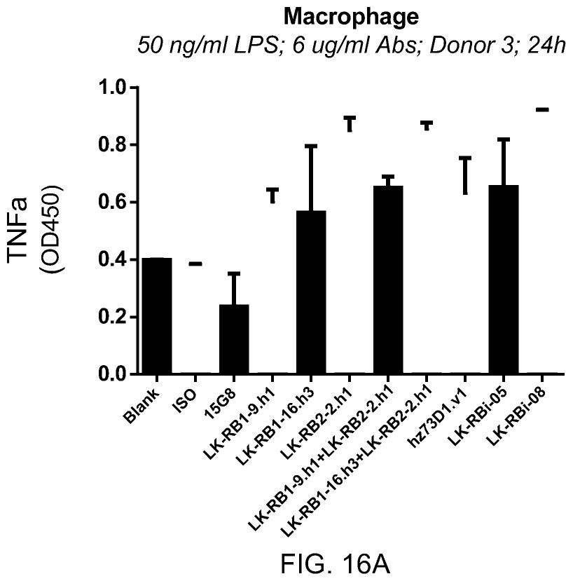

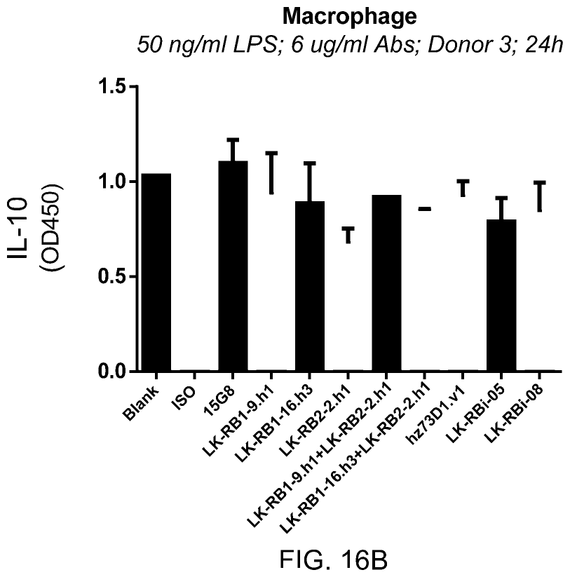

As shown in FIG. 16A, LK-RB1-9.h1, LK-RB1-16.h3, LK-RB2-2.h1, LK-RBi-05, LK-RBi-08, LK-RB1-9.h1/LK-RB2-2.h1 combination, LK-RB1-16.h3/LK-RB2-2.h1 combination and hz73D1.v1 all increased LPS-induced TNFα release. 15G8 had no effect on LPS-induced TNFα release. LK-RB2-2.h1, LK-RBi-05, LK-RBi-08, LK-RB1-9.h1/LK-RB2-2.h1 combination, LK-RB1-16.h3/LK-RB2-2.h1 combination and hz73D1.v1 also reduced LPS-induced IL-10 release in primary macrophage (FIG. 16B).

### 7.15 Example 15: Anti-LILRB1/LILRB2 Bispecific Antibodies Enhanced LPS-Induced TNFα Release in Human PBMC

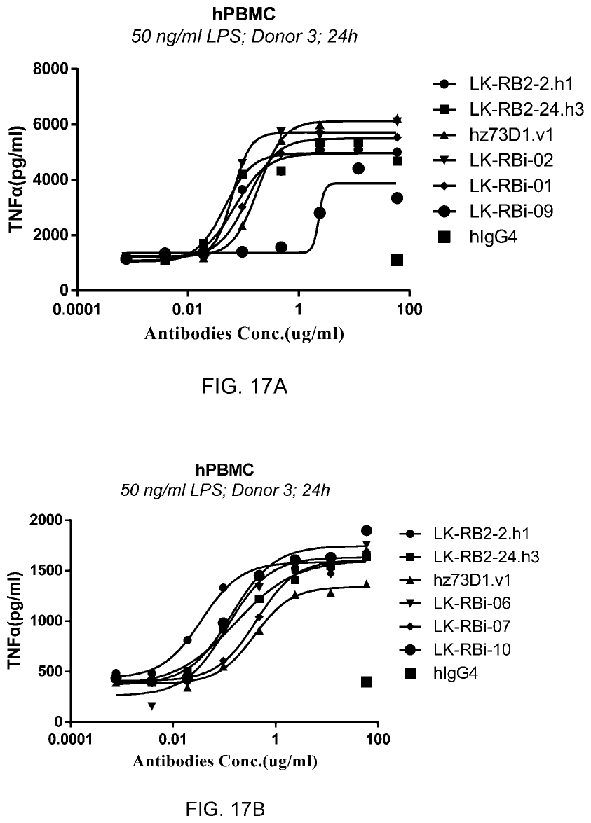

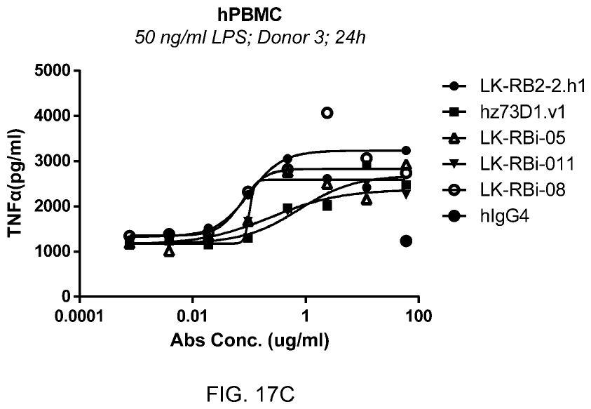

Anti-LILRB1/LILRB2 bispecific antibodies were evaluated in PBMC activation assay to understand whether they could also increase LPS-induced TNFα release in human PBMC. As shown in FIGS. 17A-17C, bispecific antibodies increased LPS-induced TNFα release in human PBMC. Bispecific antibodies (LK-RBi-01, LK-RBi-02, LK-RBi-05, LK-RBi-06, LK-RBi-07, LK-RBi-08 and LK-RBi-10) had similar activity on LPS-induced TNFα release in human PBMC comparing to anti-LILRB2 antibodies (LK-RB2-2.h1 and LK-RB2-24.h3).

### 7.16 Example 16: Anti-LILRB1/LILRB2 Bispecific Antibodies Enhanced Anti-CD3 Antibody-Induced TNFα and IFN-γ Release in Human Primary PBMC

To assess whether anti-LILRB1/LILRB2 bispecific antibodies treatment could also increase cytokine release in the presence of T cell stimulus, human primary PBMC were stimulated with anti-CD3 antibody (OKT3, BioLegend, 317325) to induce T cell activation.

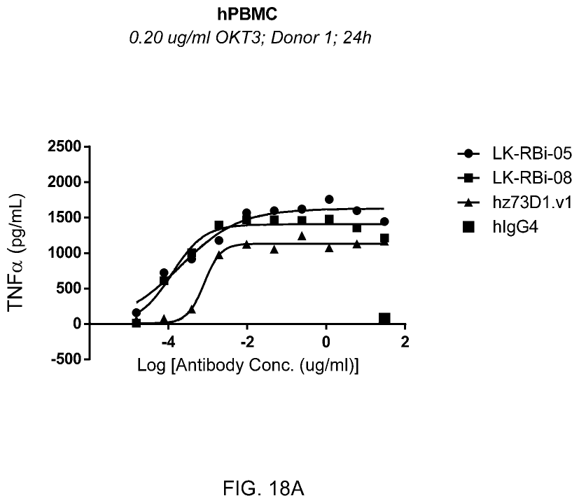

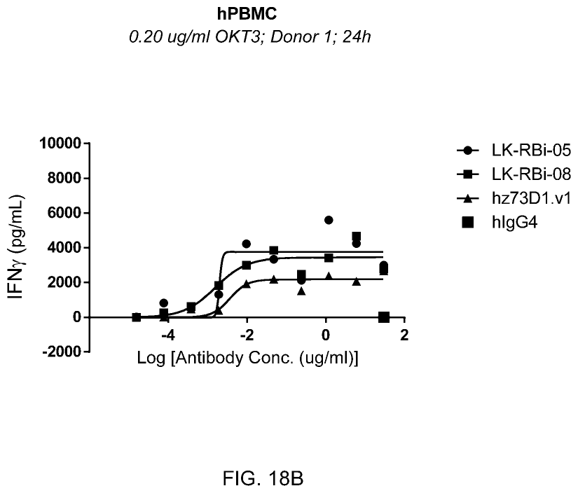

As shown in FIGS. 18A-18B, bispecific antibodies LK-RBi-05 and LK-RBi-08 could dose dependently increase OKT3-induced TNFα and IFN-γ release in human primary PBMC.

### 7.17 Example 17: Anti-LILRB1/LILRB2 Bispecific Antibodies Enhanced TNFα Release Induced by Fc Receptors (FcR) Crosslinking in Human Dendritic Cells (DC)

Activation of myeloid cells can be achieved by the crosslinking of Fc receptors (FcR) on the cell surface and the crosslinking results in cytokine production. The effect of anti-LILRB1/LILRB2 bispecific antibodies on dendritic (DC) activation was studied using monocyte derived DC (LILRB1 and LILRB2 positive, HLA-ABC positive). Briefly, 96-well plates were coated with anti-HEL1-hIgG1 antibody (5 μg/mL) and incubated overnight at 4° C. Anti-HEL-hIgG1 antibody contains an Fc domain capable of crosslinking Fc receptors on the DC cells (hDC, Milestone® Biotechnologies, PDi22061905C). DC were seeded (7×10⁴ cells/well) onto anti-HEL hIgG1 antibody coated 96 well plate, and treated with anti-LILRB1/LILRB2 bispecific antibodies, anti-LILRB1 antibodies and anti-LILRB2 antibodies. After 24 h incubation at 37° C., TNFα levels in the culture medium were measured by ELISA (Human TNF alpha ELISA Kit, Abcam, ab181421).

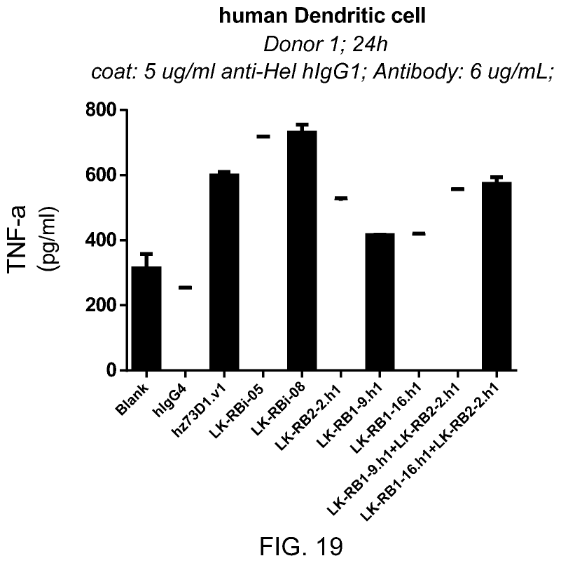

As shown in FIG. 19, anti-LILRB1 antibodies, anti-LILRB2 antibodies, anti-LILRB1/LILRB2 bispecific antibodies, LK-RB1-9.h1/LK-RB2-2.h1 combination, LK-RB1-16.h3/LK-RB2-2.h1 combination, and hz73D1.v1 all increased TNFα release in the culture medium. Bispecific antibodies induced more TNFα release than monospecific anti-LILRB1 or anti-LILRB2 antibodies. These data suggested that both LILRB1 and LILRB2 inhibited FcR-mediated activation of dendritic cells in a non-redundant way.

What is claimed:

1. An antibody or antigen binding fragment thereof that binds LILRB1, wherein the antibody or antigen binding fragment comprises:
(1). a HCDR1, a HCDR2, and a HCDR3 as set forth in a VH comprising the amino acid sequence of SEQ ID NO: 1155 and a LCDR1, a LCDR2, and a LCDR3 as set forth in a VL comprising the amino acid sequence of SEQ ID NO: 1156;
(2). a HCDR1, a HCDR2, and a HCDR3 as set forth in a VH comprising the amino acid sequence of SEQ ID NO: 1157 and a LCDR1, a LCDR2, and a LCDR3 as set forth in a VL comprising the amino acid sequence of SEQ ID NO: 1158;
(3). a HCDR1, a HCDR2, and a HCDR3 as set forth in a VH comprising the amino acid sequence of SEQ ID NO: 1159 and a LCDR1, a LCDR2, and a LCDR3 as set forth in a VL comprising the amino acid sequence of SEQ ID NO: 1160;
(4). a HCDR1, a HCDR2, and a HCDR3 as set forth in a VH comprising the amino acid sequence of SEQ ID NO: 1161 and a LCDR1, a LCDR2, and a LCDR3 as set forth in a VL comprising the amino acid sequence of SEQ ID NO: 1162;
(5). a HCDR1, a HCDR2, and a HCDR3 as set forth in a VH comprising the amino acid sequence of SEQ ID NO: 1163 and a LCDR1, a LCDR2, and a LCDR3 as set forth in a VL comprising the amino acid sequence of SEQ ID NO: 1164;
(6). a HCDR1, a HCDR2, and a HCDR3 as set forth in a VH comprising the amino acid sequence of SEQ ID NO: 1165 and a LCDR1, a LCDR2, and a LCDR3 as set forth in a VL comprising the amino acid sequence of SEQ ID NO: 1166;
(7). a HCDR1, a HCDR2, and a HCDR3 as set forth in a VH comprising the amino acid sequence of SEQ ID NO: 283 and a LCDR1, a LCDR2, and a LCDR3 as set forth in a VL comprising the amino acid sequence of SEQ ID NO: 284;
(8). a HCDR1, a HCDR2, and a HCDR3 as set forth in a VH comprising the amino acid sequence of SEQ ID NO: 285 and a LCDR1, a LCDR2, and a LCDR3 as set forth in a VL comprising the amino acid sequence of SEQ ID NO: 286;
(9). a HCDR1, a HCDR2, and a HCDR3 as set forth in a VH comprising the amino acid sequence of SEQ ID NO: 287 and a LCDR1, a LCDR2, and a LCDR3 as set forth in a VL comprising the amino acid sequence of SEQ ID NO: 288;
(10). a HCDR1, a HCDR2, and a HCDR3 as set forth in a VH comprising the amino acid sequence of SEQ ID NO: 289 and a LCDR1, a LCDR2, and a LCDR3 as set forth in a VL comprising the amino acid sequence of SEQ ID NO: 290;
(11). a HCDR1, a HCDR2, and a HCDR3 as set forth in a VH comprising the amino acid sequence of SEQ ID NO: 291 and a LCDR1, a LCDR2, and a LCDR3 as set forth in a VL comprising the amino acid sequence of SEQ ID NO: 292;
(12). a HCDR1, a HCDR2, and a HCDR3 as set forth in a VH comprising the amino acid sequence of SEQ ID NO: 293 and a LCDR1, a LCDR2, and a LCDR3 as set forth in a VL comprising the amino acid sequence of SEQ ID NO: 294;
(13). a HCDR1, a HCDR2, and a HCDR3 as set forth in a VH comprising the amino acid sequence of SEQ ID NO: 295 and a LCDR1, a LCDR2, and a LCDR3 as set forth in a VL comprising the amino acid sequence of SEQ ID NO: 296;
(14). a HCDR1, a HCDR2, and a HCDR3 as set forth in a VH comprising the amino acid sequence of SEQ ID NO: 297 and a LCDR1, a LCDR2, and a LCDR3 as set forth in a VL comprising the amino acid sequence of SEQ ID NO: 298;
(15). a HCDR1, a HCDR2, and a HCDR3 as set forth in a VH comprising the amino acid sequence of SEQ ID NO: 299 and a LCDR1, a LCDR2, and a LCDR3 as set forth in a VL comprising the amino acid sequence of SEQ ID NO: 300;
(16). a HCDR1, a HCDR2, and a HCDR3 as set forth in a VH comprising the amino acid sequence of SEQ ID NO: 301 and a LCDR1, a LCDR2, and a LCDR3 as set forth in a VL comprising the amino acid sequence of SEQ ID NO: 302;
(17). a HCDR1, a HCDR2, and a HCDR3 as set forth in a VH comprising the amino acid sequence of SEQ ID NO: 303 and a LCDR1, a LCDR2, and a LCDR3 as set forth in a VL comprising the amino acid sequence of SEQ ID NO: 304;
(18). a HCDR1, a HCDR2, and a HCDR3 as set forth in a VH comprising the amino acid sequence of SEQ ID NO: 305 and a LCDR1, a LCDR2, and a LCDR3 as set forth in a VL comprising the amino acid sequence of SEQ ID NO: 306;
(19). a HCDR1, a HCDR2, and a HCDR3 as set forth in a VH comprising the amino acid sequence of SEQ ID NO: 307 and a LCDR1, a LCDR2, and a LCDR3 as set forth in a VL comprising the amino acid sequence of SEQ ID NO: 308;
(20). a HCDR1, a HCDR2, and a HCDR3 as set forth in a VH comprising the amino acid sequence of SEQ ID NO: 309 and a LCDR1, a LCDR2, and a LCDR3 as set forth in a VL comprising the amino acid sequence of SEQ ID NO: 310;
(21). a HCDR1, a HCDR2, and a HCDR3 as set forth in a VH comprising the amino acid sequence of SEQ ID NO: 311 and a LCDR1, a LCDR2, and a LCDR3 as set forth in a VL comprising the amino acid sequence of SEQ ID NO: 312;
(22). a HCDR1, a HCDR2, and a HCDR3 as set forth in a VH comprising the amino acid sequence of SEQ ID NO: 313 and a LCDR1, a LCDR2, and a LCDR3 as set forth in a VL comprising the amino acid sequence of SEQ ID NO: 314;
(23). a HCDR1, a HCDR2, and a HCDR3 as set forth in a VH comprising the amino acid sequence of SEQ ID NO: 315 and a LCDR1, a LCDR2, and a LCDR3 as set forth in a VL comprising the amino acid sequence of SEQ ID NO: 316;
(24). a HCDR1, a HCDR2, and a HCDR3 as set forth in a VH comprising the amino acid sequence of SEQ ID NO: 317 and a LCDR1, a LCDR2, and a LCDR3 as set forth in a VL comprising the amino acid sequence of SEQ ID NO: 318;
(25). a HCDR1, a HCDR2, and a HCDR3 as set forth in a VH comprising the amino acid sequence of SEQ ID NO: 319 and a LCDR1, a LCDR2, and a LCDR3 as set forth in a VL comprising the amino acid sequence of SEQ ID NO: 320;
(26). a HCDR1, a HCDR2, and a HCDR3 as set forth in a VH comprising the amino acid sequence of SEQ ID NO: 321 and a LCDR1, a LCDR2, and a LCDR3 as set forth in a VL comprising the amino acid sequence of SEQ ID NO: 322;
(27). a HCDR1, a HCDR2, and a HCDR3 as set forth in a VH comprising the amino acid sequence of SEQ ID NO: 323 and a LCDR1, a LCDR2, and a LCDR3 as set forth in a VL comprising the amino acid sequence of SEQ ID NO: 324;
(28). a HCDR1, a HCDR2, and a HCDR3 as set forth in a VH comprising the amino acid sequence of SEQ ID NO: 325 and a LCDR1, a LCDR2, and a LCDR3 as set forth in a VL comprising the amino acid sequence of SEQ ID NO: 326;
(29). a HCDR1, a HCDR2, and a HCDR3 as set forth in a VH comprising the amino acid sequence of SEQ ID NO: 327 and a LCDR1, a LCDR2, and a LCDR3 as set forth in a VL comprising the amino acid sequence of SEQ ID NO: 328;
(30). a HCDR1, a HCDR2, and a HCDR3 as set forth in a VH comprising the amino acid sequence of SEQ ID NO: 329 and a LCDR1, a LCDR2, and a LCDR3 as set forth in a VL comprising the amino acid sequence of SEQ ID NO: 330;
(31). a HCDR1, a HCDR2, and a HCDR3 as set forth in a VH comprising the amino acid sequence of SEQ ID NO: 331 and a LCDR1, a LCDR2, and a LCDR3 as set forth in a VL comprising the amino acid sequence of SEQ ID NO: 332;
(32). a HCDR1, a HCDR2, and a HCDR3 as set forth in a VH comprising the amino acid sequence of SEQ ID NO: 333 and a LCDR1, a LCDR2, and a LCDR3 as set forth in a VL comprising the amino acid sequence of SEQ ID NO: 334;
(33). a HCDR1, a HCDR2, and a HCDR3 as set forth in a VH comprising the amino acid sequence of SEQ ID NO: 335 and a LCDR1, a LCDR2, and a LCDR3 as set forth in a VL comprising the amino acid sequence of SEQ ID NO: 336;
(34). a HCDR1, a HCDR2, and a HCDR3 as set forth in a VH comprising the amino acid sequence of SEQ ID NO: 337 and a LCDR1, a LCDR2, and a LCDR3 as set forth in a VL comprising the amino acid sequence of SEQ ID NO: 338;
(35). a HCDR1, a HCDR2, and a HCDR3 as set forth in a VH comprising the amino acid sequence of SEQ ID NO: 339 and a LCDR1, a LCDR2, and a LCDR3 as set forth in a VL comprising the amino acid sequence of SEQ ID NO: 340;
(36). a HCDR1, a HCDR2, and a HCDR3 as set forth in a VH comprising the amino acid sequence of SEQ ID NO: 341 and a LCDR1, a LCDR2, and a LCDR3 as set forth in a VL comprising the amino acid sequence of SEQ ID NO: 342;
(37). a HCDR1, a HCDR2, and a HCDR3 as set forth in a VH comprising the amino acid sequence of SEQ ID NO: 343 and a LCDR1, a LCDR2, and a LCDR3 as set forth in a VL comprising the amino acid sequence of SEQ ID NO: 344;
(38). a HCDR1, a HCDR2, and a HCDR3 as set forth in a VH comprising the amino acid sequence of SEQ ID NO: 345 and a LCDR1, a LCDR2, and a LCDR3 as set forth in a VL comprising the amino acid sequence of SEQ ID NO: 346;
(39). a HCDR1, a HCDR2, and a HCDR3 as set forth in a VH comprising the amino acid sequence of SEQ ID NO: 347 and a LCDR1, a LCDR2, and a LCDR3 as set forth in a VL comprising the amino acid sequence of SEQ ID NO: 348;
(40). a HCDR1, a HCDR2, and a HCDR3 as set forth in a VH comprising the amino acid sequence of SEQ ID NO: 349 and a LCDR1, a LCDR2, and a LCDR3 as set forth in a VL comprising the amino acid sequence of SEQ ID NO: 350;
(41). a HCDR1, a HCDR2, and a HCDR3 as set forth in a VH comprising the amino acid sequence of SEQ ID NO: 351 and a LCDR1, a LCDR2, and a LCDR3 as set forth in a VL comprising the amino acid sequence of SEQ ID NO: 352;
(42). a HCDR1, a HCDR2, and a HCDR3 as set forth in a VH comprising the amino acid sequence of SEQ ID NO: 353 and a LCDR1, a LCDR2, and a LCDR3 as set forth in a VL comprising the amino acid sequence of SEQ ID NO: 354;
(43). a HCDR1, a HCDR2, and a HCDR3 as set forth in a VH comprising the amino acid sequence of SEQ ID NO: 355 and a LCDR1, a LCDR2, and a LCDR3 as set forth in a VL comprising the amino acid sequence of SEQ ID NO: 356;
(44). a HCDR1, a HCDR2, and a HCDR3 as set forth in a VH comprising the amino acid sequence of SEQ ID NO: 357 and a LCDR1, a LCDR2, and a LCDR3 as set forth in a VL comprising the amino acid sequence of SEQ ID NO: 358;
(45). a HCDR1, a HCDR2, and a HCDR3 as set forth in a VH comprising the amino acid sequence of SEQ ID NO: 359 and a LCDR1, a LCDR2, and a LCDR3 as set forth in a VL comprising the amino acid sequence of SEQ ID NO: 360;
(46). a HCDR1, a HCDR2, and a HCDR3 as set forth in a VH comprising the amino acid sequence of SEQ ID NO: 361 and a LCDR1, a LCDR2, and a LCDR3 as set forth in a VL comprising the amino acid sequence of SEQ ID NO: 362;
(47). a HCDR1, a HCDR2, and a HCDR3 as set forth in a VH comprising the amino acid sequence of SEQ ID NO: 363 and a LCDR1, a LCDR2, and a LCDR3 as set forth in a VL comprising the amino acid sequence of SEQ ID NO: 364;
(48). a HCDR1, a HCDR2, and a HCDR3 as set forth in a VH comprising the amino acid sequence of SEQ ID NO: 365 and a LCDR1, a LCDR2, and a LCDR3 as set forth in a VL comprising the amino acid sequence of SEQ ID NO: 366;
(49). a HCDR1, a HCDR2, and a HCDR3 as set forth in a VH comprising the amino acid sequence of SEQ ID NO: 367 and a LCDR1, a LCDR2, and a LCDR3 as set forth in a VL comprising the amino acid sequence of SEQ ID NO: 368;
(50). a HCDR1, a HCDR2, and a HCDR3 as set forth in a VH comprising the amino acid sequence of SEQ ID NO: 369 and a LCDR1, a LCDR2, and a LCDR3 as set forth in a VL comprising the amino acid sequence of SEQ ID NO: 370;
(51). a HCDR1, a HCDR2, and a HCDR3 as set forth in a VH comprising the amino acid sequence of SEQ ID NO: 371 and a LCDR1, a LCDR2, and a LCDR3 as set forth in a VL comprising the amino acid sequence of SEQ ID NO: 372;
(52). a HCDR1, a HCDR2, and a HCDR3 as set forth in a VH comprising the amino acid sequence of SEQ ID NO: 373 and a LCDR1, a LCDR2, and a LCDR3 as set forth in a VL comprising the amino acid sequence of SEQ ID NO: 374;
(53). a HCDR1, a HCDR2, and a HCDR3 as set forth in a VH comprising the amino acid sequence of SEQ ID NO: 375 and a LCDR1, a LCDR2, and a LCDR3 as set forth in a VL comprising the amino acid sequence of SEQ ID NO: 376;
(54). a HCDR1, a HCDR2, and a HCDR3 as set forth in a VH comprising the amino acid sequence of SEQ ID NO: 377 and a LCDR1, a LCDR2, and a LCDR3 as set forth in a VL comprising the amino acid sequence of SEQ ID NO: 378;
(55). a HCDR1, a HCDR2, and a HCDR3 as set forth in a VH comprising the amino acid sequence of SEQ ID NO: 379 and a LCDR1, a LCDR2, and a LCDR3 as set forth in a VL comprising the amino acid sequence of SEQ ID NO: 380;
(56). a HCDR1, a HCDR2, and a HCDR3 as set forth in a VH comprising the amino acid sequence of SEQ ID NO: 381 and a LCDR1, a LCDR2, and a LCDR3 as set forth in a VL comprising the amino acid sequence of SEQ ID NO: 382;
(57). a HCDR1, a HCDR2, and a HCDR3 as set forth in a VH comprising the amino acid sequence of SEQ ID NO: 383 and a LCDR1, a LCDR2, and a LCDR3 as set forth in a VL comprising the amino acid sequence of SEQ ID NO: 384;
(58). a HCDR1, a HCDR2, and a HCDR3 as set forth in a VH comprising the amino acid sequence of SEQ ID NO: 385 and a LCDR1, a LCDR2, and a LCDR3 as set forth in a VL comprising the amino acid sequence of SEQ ID NO: 386;
(59). a HCDR1, a HCDR2, and a HCDR3 as set forth in a VH comprising the amino acid sequence of SEQ ID NO: 387 and a LCDR1, a LCDR2, and a LCDR3 as set forth in a VL comprising the amino acid sequence of SEQ ID NO: 388;
(60). a HCDR1, a HCDR2, and a HCDR3 as set forth in a VH comprising the amino acid sequence of SEQ ID NO: 389 and a LCDR1, a LCDR2, and a LCDR3 as set forth in a VL comprising the amino acid sequence of SEQ ID NO: 390;
(61). a HCDR1, a HCDR2, and a HCDR3 as set forth in a VH comprising the amino acid sequence of SEQ ID NO: 391 and a LCDR1, a LCDR2, and a LCDR3 as set forth in a VL comprising the amino acid sequence of SEQ ID NO: 392;
(62). a HCDR1, a HCDR2, and a HCDR3 as set forth in a VH comprising the amino acid sequence of SEQ ID NO: 393 and a LCDR1, a LCDR2, and a LCDR3 as set forth in a VL comprising the amino acid sequence of SEQ ID NO: 394;
(63). a HCDR1, a HCDR2, and a HCDR3 as set forth in a VH comprising the amino acid sequence of SEQ ID NO: 395 and a LCDR1, a LCDR2, and a LCDR3 as set forth in a VL comprising the amino acid sequence of SEQ ID NO: 396;
(64). a HCDR1, a HCDR2, and a HCDR3 as set forth in a VH comprising the amino acid sequence of SEQ ID NO: 397 and a LCDR1, a LCDR2, and a LCDR3 as set forth in a VL comprising the amino acid sequence of SEQ ID NO: 398; or
(65). a HCDR1, a HCDR2, and a HCDR3 as set forth in a VH comprising the amino acid sequence of SEQ ID NO: 399 and a LCDR1, a LCDR2, and a LCDR3 as set forth in a VL comprising the amino acid sequence of SEQ ID NO: 400.

2. The antibody or antigen binding fragment of claim 1, wherein:
(1). the HCDR1 comprises the amino acid sequence of SEQ ID NO: 543, the HCDR2 comprises the amino acid sequence of SEQ ID NO: 544, the HCDR3 comprises the amino acid sequence of SEQ ID NO: 1179, the LCDR1 comprises the amino acid sequence of SEQ ID NO: 546, the LCDR2 comprises the amino acid sequence of SEQ ID NO: 1281, and the LCDR3 comprises the amino acid sequence of SEQ ID NO: 548;
(2). the HCDR1 comprises the amino acid sequence of SEQ ID NO: 585, the HCDR2 comprises the amino acid sequence of SEQ ID NO: 586, the HCDR3 comprises the amino acid sequence of SEQ ID NO: 587, the LCDR1 comprises the amino acid sequence of SEQ ID NO: 588, the LCDR2 comprises the amino acid sequence of SEQ ID NO: 589, and the LCDR3 comprises the amino acid sequence of SEQ ID NO: 590;
(3). the HCDR1 comprises the amino acid sequence of SEQ ID NO: 495, the HCDR2 comprises the amino acid sequence of SEQ ID NO: 496, the HCDR3 comprises the amino acid sequence of SEQ ID NO: 497, the LCDR1 comprises the amino acid sequence of SEQ ID NO: 498, the LCDR2 comprises the amino acid sequence of SEQ ID NO: 499, and the LCDR3 comprises the amino acid sequence of SEQ ID NO: 500;
(4). the HCDR1 comprises the amino acid sequence of SEQ ID NO: 501, the HCDR2 comprises the amino acid sequence of SEQ ID NO: 502, the HCDR3 comprises the amino acid sequence of SEQ ID NO: 503, the LCDR1 comprises the amino acid sequence of SEQ ID NO: 504, the LCDR2 comprises the amino acid sequence of SEQ ID NO: 505, and the LCDR3 comprises the amino acid sequence of SEQ ID NO: 506;
(5). the HCDR1 comprises the amino acid sequence of SEQ ID NO: 507, the HCDR2 comprises the amino acid sequence of SEQ ID NO: 508, the HCDR3 comprises the amino acid sequence of SEQ ID NO: 509, the LCDR1 comprises the amino acid sequence of SEQ ID NO: 510, the LCDR2 comprises the amino acid sequence of SEQ ID NO: 511, and the LCDR3 comprises the amino acid sequence of SEQ ID NO: 512;
(6). the HCDR1 comprises the amino acid sequence of SEQ ID NO: 513, the HCDR2 comprises the amino acid sequence of SEQ ID NO: 514, the HCDR3 comprises the amino acid sequence of SEQ ID NO: 515, the LCDR1 comprises the amino acid sequence of SEQ ID NO: 516, the LCDR2 comprises the amino acid sequence of SEQ ID NO: 517, and the LCDR3 comprises the amino acid sequence of SEQ ID NO: 518;
(7). the HCDR1 comprises the amino acid sequence of SEQ ID NO: 519, the HCDR2 comprises the amino acid sequence of SEQ ID NO: 520, the HCDR3 comprises the amino acid sequence of SEQ ID NO: 521, the LCDR1 comprises the amino acid sequence of SEQ ID NO: 522, the LCDR2 comprises the amino acid sequence of SEQ ID NO: 523, and the LCDR3 comprises the amino acid sequence of SEQ ID NO: 524;
(8). the HCDR1 comprises the amino acid sequence of SEQ ID NO: 525, the HCDR2 comprises the amino acid sequence of SEQ ID NO: 526, the HCDR3 comprises the amino acid sequence of SEQ ID NO: 527, the LCDR1 comprises the amino acid sequence of SEQ ID NO: 528, the LCDR2 comprises the amino acid sequence of SEQ ID NO: 529, and the LCDR3 comprises the amino acid sequence of SEQ ID NO: 530;
(9). the HCDR1 comprises the amino acid sequence of SEQ ID NO: 531, the HCDR2 comprises the amino acid sequence of SEQ ID NO: 532, the HCDR3 comprises the amino acid sequence of SEQ ID NO: 533, the LCDR1 comprises the amino acid sequence of SEQ ID NO: 534, the LCDR2 comprises the amino acid sequence of SEQ ID NO: 535, and the LCDR3 comprises the amino acid sequence of SEQ ID NO: 536;
(10). the HCDR1 comprises the amino acid sequence of SEQ ID NO: 537, the HCDR2 comprises the amino acid sequence of SEQ ID NO: 538, the HCDR3 comprises the amino acid sequence of SEQ ID NO: 539, the LCDR1 comprises the amino acid sequence of SEQ ID NO: 540, the LCDR2 comprises the amino acid sequence of SEQ ID NO: 541, and the LCDR3 comprises the amino acid sequence of SEQ ID NO: 542;
(11). the HCDR1 comprises the amino acid sequence of SEQ ID NO: 543, the HCDR2 comprises the amino acid sequence of SEQ ID NO: 544, the HCDR3 comprises the amino acid sequence of SEQ ID NO: 545, the LCDR1 comprises the amino acid sequence of SEQ ID NO: 546, the LCDR2 comprises the amino acid sequence of SEQ ID NO: 547, and the LCDR3 comprises the amino acid sequence of SEQ ID NO: 548;
(12). the HCDR1 comprises the amino acid sequence of SEQ ID NO: 549, the HCDR2 comprises the amino acid sequence of SEQ ID NO: 550, the HCDR3 comprises the amino acid sequence of SEQ ID NO: 551, the LCDR1 comprises the amino acid sequence of SEQ ID NO: 552, the LCDR2 comprises the amino acid sequence of SEQ ID NO: 553, and the LCDR3 comprises the amino acid sequence of SEQ ID NO: 554;
(13). the HCDR1 comprises the amino acid sequence of SEQ ID NO: 555, the HCDR2 comprises the amino acid sequence of SEQ ID NO: 556, the HCDR3 comprises the amino acid sequence of SEQ ID NO: 557, the LCDR1 comprises the amino acid sequence of SEQ ID NO: 558, the LCDR2 comprises the amino acid sequence of SEQ ID NO: 559, and the LCDR3 comprises the amino acid sequence of SEQ ID NO: 560;
(14). the HCDR1 comprises the amino acid sequence of SEQ ID NO: 561, the HCDR2 comprises the amino acid sequence of SEQ ID NO: 562, the HCDR3 comprises the amino acid sequence of SEQ ID NO: 563, the LCDR1 comprises the amino acid sequence of SEQ ID NO: 564, the LCDR2 comprises the amino acid sequence of SEQ ID NO: 565, and the LCDR3 comprises the amino acid sequence of SEQ ID NO: 566;
(15). the HCDR1 comprises the amino acid sequence of SEQ ID NO: 567, the HCDR2 comprises the amino acid sequence of SEQ ID NO: 568, the HCDR3 comprises the amino acid sequence of SEQ ID NO: 569, the LCDR1 comprises the amino acid sequence of SEQ ID NO: 570, the LCDR2 comprises the amino acid sequence of SEQ ID NO: 571, and the LCDR3 comprises the amino acid sequence of SEQ ID NO: 572;
(16). the HCDR1 comprises the amino acid sequence of SEQ ID NO: 573, the HCDR2 comprises the amino acid sequence of SEQ ID NO: 574, the HCDR3 comprises the amino acid sequence of SEQ ID NO: 575, the LCDR1 comprises the amino acid sequence of SEQ ID NO: 576, the LCDR2 comprises the amino acid sequence of SEQ ID NO: 577, and the LCDR3 comprises the amino acid sequence of SEQ ID NO: 578;
(17). the HCDR1 comprises the amino acid sequence of SEQ ID NO: 579, the HCDR2 comprises the amino acid sequence of SEQ ID NO: 580, the HCDR3 comprises the amino acid sequence of SEQ ID NO: 581, the LCDR1 comprises the amino acid sequence of SEQ ID NO: 582, the LCDR2 comprises the amino acid sequence of SEQ ID NO: 583, and the LCDR3 comprises the amino acid sequence of SEQ ID NO: 584;
(18). the HCDR1 comprises the amino acid sequence of SEQ ID NO: 591, the HCDR2 comprises the amino acid sequence of SEQ ID NO: 592, the HCDR3 comprises the amino acid sequence of SEQ ID NO: 593, the LCDR1 comprises the amino acid sequence of SEQ ID NO: 594, the LCDR2 comprises the amino acid sequence of SEQ ID NO: 595, and the LCDR3 comprises the amino acid sequence of SEQ ID NO: 596;
(19). the HCDR1 comprises the amino acid sequence of SEQ ID NO: 597, the HCDR2 comprises the amino acid sequence of SEQ ID NO: 598, the HCDR3 comprises the amino acid sequence of SEQ ID NO: 599, the LCDR1 comprises the amino acid sequence of SEQ ID NO: 600, the LCDR2 comprises the amino acid sequence of SEQ ID NO: 601, and the LCDR3 comprises the amino acid sequence of SEQ ID NO: 602;
(20). the HCDR1 comprises the amino acid sequence of SEQ ID NO: 603, the HCDR2 comprises the amino acid sequence of SEQ ID NO: 604, the HCDR3 comprises the amino acid sequence of SEQ ID NO: 605, the LCDR1 comprises the amino acid sequence of SEQ ID NO: 606, the LCDR2 comprises the amino acid sequence of SEQ ID NO: 607, and the LCDR3 comprises the amino acid sequence of SEQ ID NO: 608;
(21). the HCDR1 comprises the amino acid sequence of SEQ ID NO: 609, the HCDR2 comprises the amino acid sequence of SEQ ID NO: 610, the HCDR3 comprises the amino acid sequence of SEQ ID NO: 611, the LCDR1 comprises the amino acid sequence of SEQ ID NO: 612, the LCDR2 comprises the amino acid sequence of SEQ ID NO: 613, and the LCDR3 comprises the amino acid sequence of SEQ ID NO: 614;
(22). the HCDR1 comprises the amino acid sequence of SEQ ID NO: 615, the HCDR2 comprises the amino acid sequence of SEQ ID NO: 616, the HCDR3 comprises the amino acid sequence of SEQ ID NO: 617, the LCDR1 comprises the amino acid sequence of SEQ ID NO: 618, the LCDR2 comprises the amino acid sequence of SEQ ID NO: 619, and the LCDR3 comprises the amino acid sequence of SEQ ID NO: 620;
(23). the HCDR1 comprises the amino acid sequence of SEQ ID NO: 621, the HCDR2 comprises the amino acid sequence of SEQ ID NO: 622, the HCDR3 comprises the amino acid sequence of SEQ ID NO: 623, the LCDR1 comprises the amino acid sequence of SEQ ID NO: 624, the LCDR2 comprises the amino acid sequence of SEQ ID NO: 625, and the LCDR3 comprises the amino acid sequence of SEQ ID NO: 626;
(24). the HCDR1 comprises the amino acid sequence of SEQ ID NO: 627, the HCDR2 comprises the amino acid sequence of SEQ ID NO: 628, the HCDR3 comprises the amino acid sequence of SEQ ID NO: 629, the LCDR1 comprises the amino acid sequence of SEQ ID NO: 630, the LCDR2 comprises the amino acid sequence of SEQ ID NO: 631, and the LCDR3 comprises the amino acid sequence of SEQ ID NO: 632;
(25). the HCDR1 comprises the amino acid sequence of SEQ ID NO: 633, the HCDR2 comprises the amino acid sequence of SEQ ID NO: 634, the HCDR3 comprises the amino acid sequence of SEQ ID NO: 635, the LCDR1 comprises the amino acid sequence of SEQ ID NO: 636, the LCDR2 comprises the amino acid sequence of SEQ ID NO: 637, and the LCDR3 comprises the amino acid sequence of SEQ ID NO: 638;
(26). the HCDR1 comprises the amino acid sequence of SEQ ID NO: 639, the HCDR2 comprises the amino acid sequence of SEQ ID NO: 640, the HCDR3 comprises the amino acid sequence of SEQ ID NO: 641, the LCDR1 comprises the amino acid sequence of SEQ ID NO: 642, the LCDR2 comprises the amino acid sequence of SEQ ID NO: 643, and the LCDR3 comprises the amino acid sequence of SEQ ID NO: 644;
(27). the HCDR1 comprises the amino acid sequence of SEQ ID NO: 645, the HCDR2 comprises the amino acid sequence of SEQ ID NO: 646, the HCDR3 comprises the amino acid sequence of SEQ ID NO: 647, the LCDR1 comprises the amino acid sequence of SEQ ID NO: 648, the LCDR2 comprises the amino acid sequence of SEQ ID NO: 649, and the LCDR3 comprises the amino acid sequence of SEQ ID NO: 650;
(28). the HCDR1 comprises the amino acid sequence of SEQ ID NO: 651, the HCDR2 comprises the amino acid sequence of SEQ ID NO: 652, the HCDR3 comprises the amino acid sequence of SEQ ID NO: 653, the LCDR1 comprises the amino acid sequence of SEQ ID NO: 654, the LCDR2 comprises the amino acid sequence of SEQ ID NO: 655, and the LCDR3 comprises the amino acid sequence of SEQ ID NO: 656;
(29). the HCDR1 comprises the amino acid sequence of SEQ ID NO: 657, the HCDR2 comprises the amino acid sequence of SEQ ID NO: 658, the HCDR3 comprises the amino acid sequence of SEQ ID NO: 659, the LCDR1 comprises the amino acid sequence of SEQ ID NO: 660, the LCDR2 comprises the amino acid sequence of SEQ ID NO: 661, and the LCDR3 comprises the amino acid sequence of SEQ ID NO: 662;
(30). the HCDR1 comprises the amino acid sequence of SEQ ID NO: 663, the HCDR2 comprises the amino acid sequence of SEQ ID NO: 664, the HCDR3 comprises the amino acid sequence of SEQ ID NO: 665, the LCDR1 comprises the amino acid sequence of SEQ ID NO: 666, the LCDR2 comprises the amino acid sequence of SEQ ID NO: 667, and the LCDR3 comprises the amino acid sequence of SEQ ID NO: 668;
(31). the HCDR1 comprises the amino acid sequence of SEQ ID NO: 669, the HCDR2 comprises the amino acid sequence of SEQ ID NO: 670, the HCDR3 comprises the amino acid sequence of SEQ ID NO: 671, the LCDR1 comprises the amino acid sequence of SEQ ID NO: 672, the LCDR2 comprises the amino acid sequence of SEQ ID NO: 673, and the LCDR3 comprises the amino acid sequence of SEQ ID NO: 674;
(32). the HCDR1 comprises the amino acid sequence of SEQ ID NO: 675, the HCDR2 comprises the amino acid sequence of SEQ ID NO: 676, the HCDR3 comprises the amino acid sequence of SEQ ID NO: 677, the LCDR1 comprises the amino acid sequence of SEQ ID NO: 678, the LCDR2 comprises the amino acid sequence of SEQ ID NO: 679, and the LCDR3 comprises the amino acid sequence of SEQ ID NO: 680;
(33). the HCDR1 comprises the amino acid sequence of SEQ ID NO: 681, the HCDR2 comprises the amino acid sequence of SEQ ID NO: 682, the HCDR3 comprises the amino acid sequence of SEQ ID NO: 683, the LCDR1 comprises the amino acid sequence of SEQ ID NO: 684, the LCDR2 comprises the amino acid sequence of SEQ ID NO: 685, and the LCDR3 comprises the amino acid sequence of SEQ ID NO: 686;
(34). the HCDR1 comprises the amino acid sequence of SEQ ID NO: 687, the HCDR2 comprises the amino acid sequence of SEQ ID NO: 688, the HCDR3 comprises the amino acid sequence of SEQ ID NO: 689, the LCDR1 comprises the amino acid sequence of SEQ ID NO: 690, the LCDR2 comprises the amino acid sequence of SEQ ID NO: 691, and the LCDR3 comprises the amino acid sequence of SEQ ID NO: 692;
(35). the HCDR1 comprises the amino acid sequence of SEQ ID NO: 693, the HCDR2 comprises the amino acid sequence of SEQ ID NO: 694, the HCDR3 comprises the amino acid sequence of SEQ ID NO: 695, the LCDR1 comprises the amino acid sequence of SEQ ID NO: 696, the LCDR2 comprises the amino acid sequence of SEQ ID NO: 697, and the LCDR3 comprises the amino acid sequence of SEQ ID NO: 698;
(36). the HCDR1 comprises the amino acid sequence of SEQ ID NO: 699, the HCDR2 comprises the amino acid sequence of SEQ ID NO: 700, the HCDR3 comprises the amino acid sequence of SEQ ID NO: 701, the LCDR1 comprises the amino acid sequence of SEQ ID NO: 702, the LCDR2 comprises the amino acid sequence of SEQ ID NO: 703, and the LCDR3 comprises the amino acid sequence of SEQ ID NO: 704;
(37). the HCDR1 comprises the amino acid sequence of SEQ ID NO: 705, the HCDR2 comprises the amino acid sequence of SEQ ID NO: 706, the HCDR3 comprises the amino acid sequence of SEQ ID NO: 707, the LCDR1 comprises the amino acid sequence of SEQ ID NO: 708, the LCDR2 comprises the amino acid sequence of SEQ ID NO: 709, and the LCDR3 comprises the amino acid sequence of SEQ ID NO: 710;
(38). the HCDR1 comprises the amino acid sequence of SEQ ID NO: 711, the HCDR2 comprises the amino acid sequence of SEQ ID NO: 712, the HCDR3 comprises the amino acid sequence of SEQ ID NO: 713, the LCDR1 comprises the amino acid sequence of SEQ ID NO: 714, the LCDR2 comprises the amino acid sequence of SEQ ID NO: 715, and the LCDR3 comprises the amino acid sequence of SEQ ID NO: 716;
(39). the HCDR1 comprises the amino acid sequence of SEQ ID NO: 717, the HCDR2 comprises the amino acid sequence of SEQ ID NO: 718, the HCDR3 comprises the amino acid sequence of SEQ ID NO: 719, the LCDR1 comprises the amino acid sequence of SEQ ID NO: 720, the LCDR2 comprises the amino acid sequence of SEQ ID NO: 721, and the LCDR3 comprises the amino acid sequence of SEQ ID NO: 722;
(40). the HCDR1 comprises the amino acid sequence of SEQ ID NO: 723, the HCDR2 comprises the amino acid sequence of SEQ ID NO: 724, the HCDR3 comprises the amino acid sequence of SEQ ID NO: 725, the LCDR1 comprises the amino acid sequence of SEQ ID NO: 726, the LCDR2 comprises the amino acid sequence of SEQ ID NO: 727, and the LCDR3 comprises the amino acid sequence of SEQ ID NO: 728;
(41). the HCDR1 comprises the amino acid sequence of SEQ ID NO: 729, the HCDR2 comprises the amino acid sequence of SEQ ID NO: 730, the HCDR3 comprises the amino acid sequence of SEQ ID NO: 731, the LCDR1 comprises the amino acid sequence of SEQ ID NO: 732, the LCDR2 comprises the amino acid sequence of SEQ ID NO: 733, and the LCDR3 comprises the amino acid sequence of SEQ ID NO: 734;
(42). the HCDR1 comprises the amino acid sequence of SEQ ID NO: 735, the HCDR2 comprises the amino acid sequence of SEQ ID NO: 736, the HCDR3 comprises the amino acid sequence of SEQ ID NO: 737, the LCDR1 comprises the amino acid sequence of SEQ ID NO: 738, the LCDR2 comprises the amino acid sequence of SEQ ID NO: 739, and the LCDR3 comprises the amino acid sequence of SEQ ID NO: 740;
(43). the HCDR1 comprises the amino acid sequence of SEQ ID NO: 741, the HCDR2 comprises the amino acid sequence of SEQ ID NO: 742, the HCDR3 comprises the amino acid sequence of SEQ ID NO: 743, the LCDR1 comprises the amino acid sequence of SEQ ID NO: 744, the LCDR2 comprises the amino acid sequence of SEQ ID NO: 745, and the LCDR3 comprises the amino acid sequence of SEQ ID NO: 746;
(44). the HCDR1 comprises the amino acid sequence of SEQ ID NO: 747, the HCDR2 comprises the amino acid sequence of SEQ ID NO: 748, the HCDR3 comprises the amino acid sequence of SEQ ID NO: 749, the LCDR1 comprises the amino acid sequence of SEQ ID NO: 750, the LCDR2 comprises the amino acid sequence of SEQ ID NO: 751, and the LCDR3 comprises the amino acid sequence of SEQ ID NO: 752;
(45). the HCDR1 comprises the amino acid sequence of SEQ ID NO: 753, the HCDR2 comprises the amino acid sequence of SEQ ID NO: 754, the HCDR3 comprises the amino acid sequence of SEQ ID NO: 755, the LCDR1 comprises the amino acid sequence of SEQ ID NO: 756, the LCDR2 comprises the amino acid sequence of SEQ ID NO: 757, and the LCDR3 comprises the amino acid sequence of SEQ ID NO: 758;
(46). the HCDR1 comprises the amino acid sequence of SEQ ID NO: 759, the HCDR2 comprises the amino acid sequence of SEQ ID NO: 760, the HCDR3 comprises the amino acid sequence of SEQ ID NO: 761, the LCDR1 comprises the amino acid sequence of SEQ ID NO: 762, the LCDR2 comprises the amino acid sequence of SEQ ID NO: 763, and the LCDR3 comprises the amino acid sequence of SEQ ID NO: 764;
(47). the HCDR1 comprises the amino acid sequence of SEQ ID NO: 765, the HCDR2 comprises the amino acid sequence of SEQ ID NO: 766, the HCDR3 comprises the amino acid sequence of SEQ ID NO: 767, the LCDR1 comprises the amino acid sequence of SEQ ID NO: 768, the LCDR2 comprises the amino acid sequence of SEQ ID NO: 769, and the LCDR3 comprises the amino acid sequence of SEQ ID NO: 770;
(48). the HCDR1 comprises the amino acid sequence of SEQ ID NO: 771, the HCDR2 comprises the amino acid sequence of SEQ ID NO: 772, the HCDR3 comprises the amino acid sequence of SEQ ID NO: 773, the LCDR1 comprises the amino acid sequence of SEQ ID NO: 774, the LCDR2 comprises the amino acid sequence of SEQ ID NO: 775, and the LCDR3 comprises the amino acid sequence of SEQ ID NO: 776;
(49). the HCDR1 comprises the amino acid sequence of SEQ ID NO: 777, the HCDR2 comprises the amino acid sequence of SEQ ID NO: 778, the HCDR3 comprises the amino acid sequence of SEQ ID NO: 779, the LCDR1 comprises the amino acid sequence of SEQ ID NO: 780, the LCDR2 comprises the amino acid sequence of SEQ ID NO: 781, and the LCDR3 comprises the amino acid sequence of SEQ ID NO: 782;
(50). the HCDR1 comprises the amino acid sequence of SEQ ID NO: 783, the HCDR2 comprises the amino acid sequence of SEQ ID NO: 784, the HCDR3 comprises the amino acid sequence of SEQ ID NO: 785, the LCDR1 comprises the amino acid sequence of SEQ ID NO: 786, the LCDR2 comprises the amino acid sequence of SEQ ID NO: 787, and the LCDR3 comprises the amino acid sequence of SEQ ID NO: 788;
(51). the HCDR1 comprises the amino acid sequence of SEQ ID NO: 789, the HCDR2 comprises the amino acid sequence of SEQ ID NO: 790, the HCDR3 comprises the amino acid sequence of SEQ ID NO: 791, the LCDR1 comprises the amino acid sequence of SEQ ID NO: 792, the LCDR2 comprises the amino acid sequence of SEQ ID NO: 793, and the LCDR3 comprises the amino acid sequence of SEQ ID NO: 794;
(52). the HCDR1 comprises the amino acid sequence of SEQ ID NO: 795, the HCDR2 comprises the amino acid sequence of SEQ ID NO: 796, the HCDR3 comprises the amino acid sequence of SEQ ID NO: 797, the LCDR1 comprises the amino acid sequence of SEQ ID NO: 798, the LCDR2 comprises the amino acid sequence of SEQ ID NO: 799, and the LCDR3 comprises the amino acid sequence of SEQ ID NO: 800;
(53). the HCDR1 comprises the amino acid sequence of SEQ ID NO: 801, the HCDR2 comprises the amino acid sequence of SEQ ID NO: 802, the HCDR3 comprises the amino acid sequence of SEQ ID NO: 803, the LCDR1 comprises the amino acid sequence of SEQ ID NO: 804, the LCDR2 comprises the amino acid sequence of SEQ ID NO: 805, and the LCDR3 comprises the amino acid sequence of SEQ ID NO: 806;
(54). the HCDR1 comprises the amino acid sequence of SEQ ID NO: 807, the HCDR2 comprises the amino acid sequence of SEQ ID NO: 808, the HCDR3 comprises the amino acid sequence of SEQ ID NO: 809, the LCDR1 comprises the amino acid sequence of SEQ ID NO: 810, the LCDR2 comprises the amino acid sequence of SEQ ID NO: 811, and the LCDR3 comprises the amino acid sequence of SEQ ID NO: 812;
(55). the HCDR1 comprises the amino acid sequence of SEQ ID NO: 813, the HCDR2 comprises the amino acid sequence of SEQ ID NO: 814, the HCDR3 comprises the amino acid sequence of SEQ ID NO: 815, the LCDR1 comprises the amino acid sequence of SEQ ID NO: 816, the LCDR2 comprises the amino acid sequence of SEQ ID NO: 817, and the LCDR3 comprises the amino acid sequence of SEQ ID NO: 818;
(56). the HCDR1 comprises the amino acid sequence of SEQ ID NO: 819, the HCDR2 comprises the amino acid sequence of SEQ ID NO: 820, the HCDR3 comprises the amino acid sequence of SEQ ID NO: 821, the LCDR1 comprises the amino acid sequence of SEQ ID NO: 822, the LCDR2 comprises the amino acid sequence of SEQ ID NO: 823, and the LCDR3 comprises the amino acid sequence of SEQ ID NO: 824;
(57). the HCDR1 comprises the amino acid sequence of SEQ ID NO: 825, the HCDR2 comprises the amino acid sequence of SEQ ID NO: 826, the HCDR3 comprises the amino acid sequence of SEQ ID NO: 827, the LCDR1 comprises the amino acid sequence of SEQ ID NO: 828, the LCDR2 comprises the amino acid sequence of SEQ ID NO: 829, and the LCDR3 comprises the amino acid sequence of SEQ ID NO: 830;
(58). the HCDR1 comprises the amino acid sequence of SEQ ID NO: 831, the HCDR2 comprises the amino acid sequence of SEQ ID NO: 832, the HCDR3 comprises the amino acid sequence of SEQ ID NO: 833, the LCDR1 comprises the amino acid sequence of SEQ ID NO: 834, the LCDR2 comprises the amino acid sequence of SEQ ID NO: 835, and the LCDR3 comprises the amino acid sequence of SEQ ID NO: 836;
(59). the HCDR1 comprises the amino acid sequence of SEQ ID NO: 837, the HCDR2 comprises the amino acid sequence of SEQ ID NO: 838, the HCDR3 comprises the amino acid sequence of SEQ ID NO: 839, the LCDR1 comprises the amino acid sequence of SEQ ID NO: 840, the LCDR2 comprises the amino acid sequence of SEQ ID NO: 841, and the LCDR3 comprises the amino acid sequence of SEQ ID NO: 842; or
(60). the HCDR1 comprises the amino acid sequence of SEQ ID NO: 843, the HCDR2 comprises the amino acid sequence of SEQ ID NO: 844, the HCDR3 comprises the amino acid sequence of SEQ ID NO: 845, the LCDR1 comprises the amino acid sequence of SEQ ID NO: 846, the LCDR2 comprises the amino acid sequence of SEQ ID NO: 847, and the LCDR3 comprises the amino acid sequence of SEQ ID NO: 848.

3. The antibody or antigen binding fragment of claim 1 or 2, comprises:
(1). a VH comprising the amino acid sequence of SEQ ID NO: 1155 and a VL comprising the amino acid sequence of SEQ ID NO: 1156;
(2). a VH comprising the amino acid sequence of SEQ ID NO: 1157 and a VL comprising the amino acid sequence of SEQ ID NO: 1158;
(3). a VH comprising the amino acid sequence of SEQ ID NO: 1159 and a VL comprising the amino acid sequence of SEQ ID NO: 1160;
(4). a VH comprising the amino acid sequence of SEQ ID NO: 1161 and a VL comprising the amino acid sequence of SEQ ID NO: 1162;
(5). a VH comprising the amino acid sequence of SEQ ID NO: 1163 and a VL comprising the amino acid sequence of SEQ ID NO: 1164;
(6). a VH comprising the amino acid sequence of SEQ ID NO: 1165 and a VL comprising the amino acid sequence of SEQ ID NO: 1166.
(7). a VH comprising the amino acid sequence of SEQ ID NO: 283 and a VL comprising the amino acid sequence of SEQ ID NO: 284;
(8). a VH comprising the amino acid sequence of SEQ ID NO: 285 and a VL comprising the amino acid sequence of SEQ ID NO: 286;
(9). a VH comprising the amino acid sequence of SEQ ID NO: 287 and a VL comprising the amino acid sequence of SEQ ID NO: 288;
(10). a VH comprising the amino acid sequence of SEQ ID NO: 289 and a VL comprising the amino acid sequence of SEQ ID NO: 290;
(11). a VH comprising the amino acid sequence of SEQ ID NO: 291 and a VL comprising the amino acid sequence of SEQ ID NO: 292;
(12). a VH comprising the amino acid sequence of SEQ ID NO: 293 and a VL comprising the amino acid sequence of SEQ ID NO: 294;
(13). a VH comprising the amino acid sequence of SEQ ID NO: 295 and a VL comprising the amino acid sequence of SEQ ID NO: 296;
(14). a VH comprising the amino acid sequence of SEQ ID NO: 297 and a VL comprising the amino acid sequence of SEQ ID NO: 298;
(15). a VH comprising the amino acid sequence of SEQ ID NO: 299 and a VL comprising the amino acid sequence of SEQ ID NO: 300;
(16). a VH comprising the amino acid sequence of SEQ ID NO: 301 and a VL comprising the amino acid sequence of SEQ ID NO: 302;
(17). a VH comprising the amino acid sequence of SEQ ID NO: 303 and a VL comprising the amino acid sequence of SEQ ID NO: 304;
(18). a VH comprising the amino acid sequence of SEQ ID NO: 305 and a VL comprising the amino acid sequence of SEQ ID NO: 306;
(19). a VH comprising the amino acid sequence of SEQ ID NO: 307 and a VL comprising the amino acid sequence of SEQ ID NO: 308;
(20). a VH comprising the amino acid sequence of SEQ ID NO: 309 and a VL comprising the amino acid sequence of SEQ ID NO: 310;
(21). a VH comprising the amino acid sequence of SEQ ID NO: 311 and a VL comprising the amino acid sequence of SEQ ID NO: 312;
(22). a VH comprising the amino acid sequence of SEQ ID NO: 313 and a VL comprising the amino acid sequence of SEQ ID NO: 314;
(23). a VH comprising the amino acid sequence of SEQ ID NO: 315 and a VL comprising the amino acid sequence of SEQ ID NO: 316;
(24). a VH comprising the amino acid sequence of SEQ ID NO: 317 and a VL comprising the amino acid sequence of SEQ ID NO: 318;
(25). a VH comprising the amino acid sequence of SEQ ID NO: 319 and a VL comprising the amino acid sequence of SEQ ID NO: 320;
(26). a VH comprising the amino acid sequence of SEQ ID NO: 321 and a VL comprising the amino acid sequence of SEQ ID NO: 322;
(27). a VH comprising the amino acid sequence of SEQ ID NO: 323 and a VL comprising the amino acid sequence of SEQ ID NO: 324;
(28). a VH comprising the amino acid sequence of SEQ ID NO: 325 and a VL comprising the amino acid sequence of SEQ ID NO: 326;
(29). a VH comprising the amino acid sequence of SEQ ID NO: 327 and a VL comprising the amino acid sequence of SEQ ID NO: 328;
(30). a VH comprising the amino acid sequence of SEQ ID NO: 329 and a VL comprising the amino acid sequence of SEQ ID NO: 330;
(31). a VH comprising the amino acid sequence of SEQ ID NO: 331 and a VL comprising the amino acid sequence of SEQ ID NO: 332;
(32). a VH comprising the amino acid sequence of SEQ ID NO: 333 and a VL comprising the amino acid sequence of SEQ ID NO: 334;
(33). a VH comprising the amino acid sequence of SEQ ID NO: 335 and a VL comprising the amino acid sequence of SEQ ID NO: 336;
(34). a VH comprising the amino acid sequence of SEQ ID NO: 337 and a VL comprising the amino acid sequence of SEQ ID NO: 338;
(35). a VH comprising the amino acid sequence of SEQ ID NO: 339 and a VL comprising the amino acid sequence of SEQ ID NO: 340;
(36). a VH comprising the amino acid sequence of SEQ ID NO: 341 and a VL comprising the amino acid sequence of SEQ ID NO: 342;
(37). a VH comprising the amino acid sequence of SEQ ID NO: 343 and a VL comprising the amino acid sequence of SEQ ID NO: 344;
(38). a VH comprising the amino acid sequence of SEQ ID NO: 345 and a VL comprising the amino acid sequence of SEQ ID NO: 346;
(39). a VH comprising the amino acid sequence of SEQ ID NO: 347 and a VL comprising the amino acid sequence of SEQ ID NO: 348;
(40). a VH comprising the amino acid sequence of SEQ ID NO: 349 and a VL comprising the amino acid sequence of SEQ ID NO: 350;
(41). a VH comprising the amino acid sequence of SEQ ID NO: 351 and a VL comprising the amino acid sequence of SEQ ID NO: 352;
(42). a VH comprising the amino acid sequence of SEQ ID NO: 353 and a VL comprising the amino acid sequence of SEQ ID NO: 354;
(43). a VH comprising the amino acid sequence of SEQ ID NO: 355 and a VL comprising the amino acid sequence of SEQ ID NO: 356;
(44). a VH comprising the amino acid sequence of SEQ ID NO: 357 and a VL comprising the amino acid sequence of SEQ ID NO: 358;
(45). a VH comprising the amino acid sequence of SEQ ID NO: 359 and a VL comprising the amino acid sequence of SEQ ID NO: 360;
(46). a VH comprising the amino acid sequence of SEQ ID NO: 361 and a VL comprising the amino acid sequence of SEQ ID NO: 362;
(47). a VH comprising the amino acid sequence of SEQ ID NO: 363 and a VL comprising the amino acid sequence of SEQ ID NO: 364;
(48). a VH comprising the amino acid sequence of SEQ ID NO: 365 and a VL comprising the amino acid sequence of SEQ ID NO: 366;
(49). a VH comprising the amino acid sequence of SEQ ID NO: 367 and a VL comprising the amino acid sequence of SEQ ID NO: 368;
(50). a VH comprising the amino acid sequence of SEQ ID NO: 369 and a VL comprising the amino acid sequence of SEQ ID NO: 370;
(51). a VH comprising the amino acid sequence of SEQ ID NO: 371 and a VL comprising the amino acid sequence of SEQ ID NO: 372;
(52). a VH comprising the amino acid sequence of SEQ ID NO: 373 and a VL comprising the amino acid sequence of SEQ ID NO: 374;
(53). a VH comprising the amino acid sequence of SEQ ID NO: 375 and a VL comprising the amino acid sequence of SEQ ID NO: 376;
(54). a VH comprising the amino acid sequence of SEQ ID NO: 377 and a VL comprising the amino acid sequence of SEQ ID NO: 378;
(55). a VH comprising the amino acid sequence of SEQ ID NO: 379 and a VL comprising the amino acid sequence of SEQ ID NO: 380;
(56). a VH comprising the amino acid sequence of SEQ ID NO: 381 and a VL comprising the amino acid sequence of SEQ ID NO: 382;
(57). a VH comprising the amino acid sequence of SEQ ID NO: 383 and a VL comprising the amino acid sequence of SEQ ID NO: 384;
(58). a VH comprising the amino acid sequence of SEQ ID NO: 385 and a VL comprising the amino acid sequence of SEQ ID NO: 386;
(59). a VH comprising the amino acid sequence of SEQ ID NO: 387 and a VL comprising the amino acid sequence of SEQ ID NO: 388;
(60). a VH comprising the amino acid sequence of SEQ ID NO: 389 and a VL comprising the amino acid sequence of SEQ ID NO: 390;
(61). a VH comprising the amino acid sequence of SEQ ID NO: 391 and a VL comprising the amino acid sequence of SEQ ID NO: 392;
(62). a VH comprising the amino acid sequence of SEQ ID NO: 393 and a VL comprising the amino acid sequence of SEQ ID NO: 394;
(63). a VH comprising the amino acid sequence of SEQ ID NO: 395 and a VL comprising the amino acid sequence of SEQ ID NO: 396;
(64). a VH comprising the amino acid sequence of SEQ ID NO: 397 and a VL comprising the amino acid sequence of SEQ ID NO: 398; or
(65). a VH comprising the amino acid sequence of SEQ ID NO: 399 and a VL comprising the amino acid sequence of SEQ ID NO: 400.

4. An antibody or antigen binding fragment thereof that binds LILRB2, wherein the antibody or antigen binding fragment comprises:
(1). a HCDR1, a HCDR2, and a HCDR3 as set forth in a VH comprising the amino acid sequence of SEQ ID NO: 1167 and a LCDR1, a LCDR2, and a LCDR3 as set forth in a VL comprising the amino acid sequence of SEQ ID NO: 1168;
(2). a HCDR1, a HCDR2, and a HCDR3 as set forth in a VH comprising the amino acid sequence of SEQ ID NO: 1169 and a LCDR1, a LCDR2, and a LCDR3 as set forth in a VL comprising the amino acid sequence of SEQ ID NO: 1170;
(3). a HCDR1, a HCDR2, and a HCDR3 as set forth in a VH comprising the amino acid sequence of SEQ ID NO: 1171 and a LCDR1, a LCDR2, and a LCDR3 as set forth in a VL comprising the amino acid sequence of SEQ ID NO: 1172;
(4). a HCDR1, a HCDR2, and a HCDR3 as set forth in a VH comprising the amino acid sequence of SEQ ID NO: 1173 and a LCDR1, a LCDR2, and a LCDR3 as set forth in a VL comprising the amino acid sequence of SEQ ID NO: 1174;
(5). a HCDR1, a HCDR2, and a HCDR3 as set forth in a VH comprising the amino acid sequence of SEQ ID NO: 1175 and a LCDR1, a LCDR2, and a LCDR3 as set forth in a VL comprising the amino acid sequence of SEQ ID NO: 1176;
(6). a HCDR1, a HCDR2, and a HCDR3 as set forth in a VH comprising the amino acid sequence of SEQ ID NO: 1177 and a LCDR1, a LCDR2, and a LCDR3 as set forth in a VL comprising the amino acid sequence of SEQ ID NO: 1178;
(7). a HCDR1, a HCDR2, and a HCDR3 as set forth in a VH comprising the amino acid sequence of SEQ ID NO: 401 and a LCDR1, a LCDR2, and a LCDR3 as set forth in a VL comprising the amino acid sequence of SEQ ID NO: 402;
(8). a HCDR1, a HCDR2, and a HCDR3 as set forth in a VH comprising the amino acid sequence of SEQ ID NO: 403 and a LCDR1, a LCDR2, and a LCDR3 as set forth in a VL comprising the amino acid sequence of SEQ ID NO: 404;
(9). a HCDR1, a HCDR2, and a HCDR3 as set forth in a VH comprising the amino acid sequence of SEQ ID NO: 405 and a LCDR1, a LCDR2, and a LCDR3 as set forth in a VL comprising the amino acid sequence of SEQ ID NO: 406;
(10). a HCDR1, a HCDR2, and a HCDR3 as set forth in a VH comprising the amino acid sequence of SEQ ID NO: 407 and a LCDR1, a LCDR2, and a LCDR3 as set forth in a VL comprising the amino acid sequence of SEQ ID NO: 408;
(11). a HCDR1, a HCDR2, and a HCDR3 as set forth in a VH comprising the amino acid sequence of SEQ ID NO: 409 and a LCDR1, a LCDR2, and a LCDR3 as set forth in a VL comprising the amino acid sequence of SEQ ID NO: 410;
(12). a HCDR1, a HCDR2, and a HCDR3 as set forth in a VH comprising the amino acid sequence of SEQ ID NO: 411 and a LCDR1, a LCDR2, and a LCDR3 as set forth in a VL comprising the amino acid sequence of SEQ ID NO: 412;
(13). a HCDR1, a HCDR2, and a HCDR3 as set forth in a VH comprising the amino acid sequence of SEQ ID NO: 413 and a LCDR1, a LCDR2, and a LCDR3 as set forth in a VL comprising the amino acid sequence of SEQ ID NO: 414;
(14). a HCDR1, a HCDR2, and a HCDR3 as set forth in a VH comprising the amino acid sequence of SEQ ID NO: 415 and a LCDR1, a LCDR2, and a LCDR3 as set forth in a VL comprising the amino acid sequence of SEQ ID NO: 416;
(15). a HCDR1, a HCDR2, and a HCDR3 as set forth in a VH comprising the amino acid sequence of SEQ ID NO: 417 and a LCDR1, a LCDR2, and a LCDR3 as set forth in a VL comprising the amino acid sequence of SEQ ID NO: 418;
(16). a HCDR1, a HCDR2, and a HCDR3 as set forth in a VH comprising the amino acid sequence of SEQ ID NO: 419 and a LCDR1, a LCDR2, and a LCDR3 as set forth in a VL comprising the amino acid sequence of SEQ ID NO: 420;
(17). a HCDR1, a HCDR2, and a HCDR3 as set forth in a VH comprising the amino acid sequence of SEQ ID NO: 421 and a LCDR1, a LCDR2, and a LCDR3 as set forth in a VL comprising the amino acid sequence of SEQ ID NO: 422;
(18). a HCDR1, a HCDR2, and a HCDR3 as set forth in a VH comprising the amino acid sequence of SEQ ID NO: 423 and a LCDR1, a LCDR2, and a LCDR3 as set forth in a VL comprising the amino acid sequence of SEQ ID NO: 424;
(19). a HCDR1, a HCDR2, and a HCDR3 as set forth in a VH comprising the amino acid sequence of SEQ ID NO: 425 and a LCDR1, a LCDR2, and a LCDR3 as set forth in a VL comprising the amino acid sequence of SEQ ID NO: 426;
(20). a HCDR1, a HCDR2, and a HCDR3 as set forth in a VH comprising the amino acid sequence of SEQ ID NO: 427 and a LCDR1, a LCDR2, and a LCDR3 as set forth in a VL comprising the amino acid sequence of SEQ ID NO: 428;
(21). a HCDR1, a HCDR2, and a HCDR3 as set forth in a VH comprising the amino acid sequence of SEQ ID NO: 429 and a LCDR1, a LCDR2, and a LCDR3 as set forth in a VL comprising the amino acid sequence of SEQ ID NO: 430;
(22). a HCDR1, a HCDR2, and a HCDR3 as set forth in a VH comprising the amino acid sequence of SEQ ID NO: 431 and a LCDR1, a LCDR2, and a LCDR3 as set forth in a VL comprising the amino acid sequence of SEQ ID NO: 432;
(23). a HCDR1, a HCDR2, and a HCDR3 as set forth in a VH comprising the amino acid sequence of SEQ ID NO: 433 and a LCDR1, a LCDR2, and a LCDR3 as set forth in a VL comprising the amino acid sequence of SEQ ID NO: 434;
(24). a HCDR1, a HCDR2, and a HCDR3 as set forth in a VH comprising the amino acid sequence of SEQ ID NO: 435 and a LCDR1, a LCDR2, and a LCDR3 as set forth in a VL comprising the amino acid sequence of SEQ ID NO: 436;
(25). a HCDR1, a HCDR2, and a HCDR3 as set forth in a VH comprising the amino acid sequence of SEQ ID NO: 437 and a LCDR1, a LCDR2, and a LCDR3 as set forth in a VL comprising the amino acid sequence of SEQ ID NO: 438;
(26). a HCDR1, a HCDR2, and a HCDR3 as set forth in a VH comprising the amino acid sequence of SEQ ID NO: 439 and a LCDR1, a LCDR2, and a LCDR3 as set forth in a VL comprising the amino acid sequence of SEQ ID NO: 440;
(27). a HCDR1, a HCDR2, and a HCDR3 as set forth in a VH comprising the amino acid sequence of SEQ ID NO: 441 and a LCDR1, a LCDR2, and a LCDR3 as set forth in a VL comprising the amino acid sequence of SEQ ID NO: 442;
(28). a HCDR1, a HCDR2, and a HCDR3 as set forth in a VH comprising the amino acid sequence of SEQ ID NO: 443 and a LCDR1, a LCDR2, and a LCDR3 as set forth in a VL comprising the amino acid sequence of SEQ ID NO: 444;
(29). a HCDR1, a HCDR2, and a HCDR3 as set forth in a VH comprising the amino acid sequence of SEQ ID NO: 445 and a LCDR1, a LCDR2, and a LCDR3 as set forth in a VL comprising the amino acid sequence of SEQ ID NO: 446;
(30). a HCDR1, a HCDR2, and a HCDR3 as set forth in a VH comprising the amino acid sequence of SEQ ID NO: 447 and a LCDR1, a LCDR2, and a LCDR3 as set forth in a VL comprising the amino acid sequence of SEQ ID NO: 448;
(31). a HCDR1, a HCDR2, and a HCDR3 as set forth in a VH comprising the amino acid sequence of SEQ ID NO: 449 and a LCDR1, a LCDR2, and a LCDR3 as set forth in a VL comprising the amino acid sequence of SEQ ID NO: 450;
(32). a HCDR1, a HCDR2, and a HCDR3 as set forth in a VH comprising the amino acid sequence of SEQ ID NO: 451 and a LCDR1, a LCDR2, and a LCDR3 as set forth in a VL comprising the amino acid sequence of SEQ ID NO: 452;
(33). a HCDR1, a HCDR2, and a HCDR3 as set forth in a VH comprising the amino acid sequence of SEQ ID NO: 453 and a LCDR1, a LCDR2, and a LCDR3 as set forth in a VL comprising the amino acid sequence of SEQ ID NO: 454;
(34). a HCDR1, a HCDR2, and a HCDR3 as set forth in a VH comprising the amino acid sequence of SEQ ID NO: 455 and a LCDR1, a LCDR2, and a LCDR3 as set forth in a VL comprising the amino acid sequence of SEQ ID NO: 456;
(35). a HCDR1, a HCDR2, and a HCDR3 as set forth in a VH comprising the amino acid sequence of SEQ ID NO: 457 and a LCDR1, a LCDR2, and a LCDR3 as set forth in a VL comprising the amino acid sequence of SEQ ID NO: 458;
(36). a HCDR1, a HCDR2, and a HCDR3 as set forth in a VH comprising the amino acid sequence of SEQ ID NO: 459 and a LCDR1, a LCDR2, and a LCDR3 as set forth in a VL comprising the amino acid sequence of SEQ ID NO: 460;
(37). a HCDR1, a HCDR2, and a HCDR3 as set forth in a VH comprising the amino acid sequence of SEQ ID NO: 461 and a LCDR1, a LCDR2, and a LCDR3 as set forth in a VL comprising the amino acid sequence of SEQ ID NO: 462;
(38). a HCDR1, a HCDR2, and a HCDR3 as set forth in a VH comprising the amino acid sequence of SEQ ID NO: 463 and a LCDR1, a LCDR2, and a LCDR3 as set forth in a VL comprising the amino acid sequence of SEQ ID NO: 464;
(39). a HCDR1, a HCDR2, and a HCDR3 as set forth in a VH comprising the amino acid sequence of SEQ ID NO: 465 and a LCDR1, a LCDR2, and a LCDR3 as set forth in a VL comprising the amino acid sequence of SEQ ID NO: 466;
(40). a HCDR1, a HCDR2, and a HCDR3 as set forth in a VH comprising the amino acid sequence of SEQ ID NO: 467 and a LCDR1, a LCDR2, and a LCDR3 as set forth in a VL comprising the amino acid sequence of SEQ ID NO: 468;
(41). a HCDR1, a HCDR2, and a HCDR3 as set forth in a VH comprising the amino acid sequence of SEQ ID NO: 469 and a LCDR1, a LCDR2, and a LCDR3 as set forth in a VL comprising the amino acid sequence of SEQ ID NO: 470;
(42). a HCDR1, a HCDR2, and a HCDR3 as set forth in a VH comprising the amino acid sequence of SEQ ID NO: 471 and a LCDR1, a LCDR2, and a LCDR3 as set forth in a VL comprising the amino acid sequence of SEQ ID NO: 472;
(43). a HCDR1, a HCDR2, and a HCDR3 as set forth in a VH comprising the amino acid sequence of SEQ ID NO: 473 and a LCDR1, a LCDR2, and a LCDR3 as set forth in a VL comprising the amino acid sequence of SEQ ID NO: 474;
(44). a HCDR1, a HCDR2, and a HCDR3 as set forth in a VH comprising the amino acid sequence of SEQ ID NO: 475 and a LCDR1, a LCDR2, and a LCDR3 as set forth in a VL comprising the amino acid sequence of SEQ ID NO: 476;
(45). a HCDR1, a HCDR2, and a HCDR3 as set forth in a VH comprising the amino acid sequence of SEQ ID NO: 477 and a LCDR1, a LCDR2, and a LCDR3 as set forth in a VL comprising the amino acid sequence of SEQ ID NO: 478;
(46). a HCDR1, a HCDR2, and a HCDR3 as set forth in a VH comprising the amino acid sequence of SEQ ID NO: 479 and a LCDR1, a LCDR2, and a LCDR3 as set forth in a VL comprising the amino acid sequence of SEQ ID NO: 480;
(47). a HCDR1, a HCDR2, and a HCDR3 as set forth in a VH comprising the amino acid sequence of SEQ ID NO: 481 and a LCDR1, a LCDR2, and a LCDR3 as set forth in a VL comprising the amino acid sequence of SEQ ID NO: 482;
(48). a HCDR1, a HCDR2, and a HCDR3 as set forth in a VH comprising the amino acid sequence of SEQ ID NO: 483 and a LCDR1, a LCDR2, and a LCDR3 as set forth in a VL comprising the amino acid sequence of SEQ ID NO: 484;
(49). a HCDR1, a HCDR2, and a HCDR3 as set forth in a VH comprising the amino acid sequence of SEQ ID NO: 485 and a LCDR1, a LCDR2, and a LCDR3 as set forth in a VL comprising the amino acid sequence of SEQ ID NO: 486;
(50). a HCDR1, a HCDR2, and a HCDR3 as set forth in a VH comprising the amino acid sequence of SEQ ID NO: 487 and a LCDR1, a LCDR2, and a LCDR3 as set forth in a VL comprising the amino acid sequence of SEQ ID NO: 488;
(51). a HCDR1, a HCDR2, and a HCDR3 as set forth in a VH comprising the amino acid sequence of SEQ ID NO: 489 and a LCDR1, a LCDR2, and a LCDR3 as set forth in a VL comprising the amino acid sequence of SEQ ID NO: 490;
(52). a HCDR1, a HCDR2, and a HCDR3 as set forth in a VH comprising the amino acid sequence of SEQ ID NO: 491 and a LCDR1, a LCDR2, and a LCDR3 as set forth in a VL comprising the amino acid sequence of SEQ ID NO: 492; or
(53). a HCDR1, a HCDR2, and a HCDR3 as set forth in a VH comprising the amino acid sequence of SEQ ID NO: 493 and a LCDR1, a LCDR2, and a LCDR3 as set forth in a VL comprising the amino acid sequence of SEQ ID NO: 494.

5. The antibody or antigen binding fragment of claim 4, wherein:
(1). the HCDR1 comprises the amino acid sequence of SEQ ID NO: 855, the HCDR2 comprises the amino acid sequence of SEQ ID NO: 856, the HCDR3 comprises the amino acid sequence of SEQ ID NO: 857, the LCDR1 comprises the amino acid sequence of SEQ ID NO: 858, the LCDR2 comprises the amino acid sequence of SEQ ID NO: 1282, and the LCDR3 comprises the amino acid sequence of SEQ ID NO: 860;
(2). the HCDR1 comprises the amino acid sequence of SEQ ID NO: 987, the HCDR2 comprises the amino acid sequence of SEQ ID NO: 988, the HCDR3 comprises the amino acid sequence of SEQ ID NO: 989, the LCDR1 comprises the amino acid sequence of SEQ ID NO: 990, the LCDR2 comprises the amino acid sequence of SEQ ID NO: 991, and the LCDR3 comprises the amino acid sequence of SEQ ID NO: 992;
(3). the HCDR1 comprises the amino acid sequence of SEQ ID NO: 849, the HCDR2 comprises the amino acid sequence of SEQ ID NO: 850, the HCDR3 comprises the amino acid sequence of SEQ ID NO: 851, the LCDR1 comprises the amino acid sequence of SEQ ID NO: 852, the LCDR2 comprises the amino acid sequence of SEQ ID NO: 853, and the LCDR3 comprises the amino acid sequence of SEQ ID NO: 854;
(4). the HCDR1 comprises the amino acid sequence of SEQ ID NO: 855, the HCDR2 comprises the amino acid sequence of SEQ ID NO: 856, the HCDR3 comprises the amino acid sequence of SEQ ID NO: 857, the LCDR1 comprises the amino acid sequence of SEQ ID NO: 858, the LCDR2 comprises the amino acid sequence of SEQ ID NO: 859, and the LCDR3 comprises the amino acid sequence of SEQ ID NO: 860;
(5). the HCDR1 comprises the amino acid sequence of SEQ ID NO: 861, the HCDR2 comprises the amino acid sequence of SEQ ID NO: 862, the HCDR3 comprises the amino acid sequence of SEQ ID NO: 863, the LCDR1 comprises the amino acid sequence of SEQ ID NO: 864, the LCDR2 comprises the amino acid sequence of SEQ ID NO: 865, and the LCDR3 comprises the amino acid sequence of SEQ ID NO: 866;
(6). the HCDR1 comprises the amino acid sequence of SEQ ID NO: 867, the HCDR2 comprises the amino acid sequence of SEQ ID NO: 868, the HCDR3 comprises the amino acid sequence of SEQ ID NO: 869, the LCDR1 comprises the amino acid sequence of SEQ ID NO: 870, the LCDR2 comprises the amino acid sequence of SEQ ID NO: 871, and the LCDR3 comprises the amino acid sequence of SEQ ID NO: 872;
(7). the HCDR1 comprises the amino acid sequence of SEQ ID NO: 873, the HCDR2 comprises the amino acid sequence of SEQ ID NO: 874, the HCDR3 comprises the amino acid sequence of SEQ ID NO: 875, the LCDR1 comprises the amino acid sequence of SEQ ID NO: 876, the LCDR2 comprises the amino acid sequence of SEQ ID NO: 877, and the LCDR3 comprises the amino acid sequence of SEQ ID NO: 878;
(8). the HCDR1 comprises the amino acid sequence of SEQ ID NO: 879, the HCDR2 comprises the amino acid sequence of SEQ ID NO: 880, the HCDR3 comprises the amino acid sequence of SEQ ID NO: 881, the LCDR1 comprises the amino acid sequence of SEQ ID NO: 882, the LCDR2 comprises the amino acid sequence of SEQ ID NO: 883, and the LCDR3 comprises the amino acid sequence of SEQ ID NO: 884;
(9). the HCDR1 comprises the amino acid sequence of SEQ ID NO: 885, the HCDR2 comprises the amino acid sequence of SEQ ID NO: 886, the HCDR3 comprises the amino acid sequence of SEQ ID NO: 887, the LCDR1 comprises the amino acid sequence of SEQ ID NO: 888, the LCDR2 comprises the amino acid sequence of SEQ ID NO: 889, and the LCDR3 comprises the amino acid sequence of SEQ ID NO: 890;
(10). the HCDR1 comprises the amino acid sequence of SEQ ID NO: 891, the HCDR2 comprises the amino acid sequence of SEQ ID NO: 892, the HCDR3 comprises the amino acid sequence of SEQ ID NO: 893, the LCDR1 comprises the amino acid sequence of SEQ ID NO: 894, the LCDR2 comprises the amino acid sequence of SEQ ID NO: 895, and the LCDR3 comprises the amino acid sequence of SEQ ID NO: 896;
(11). the HCDR1 comprises the amino acid sequence of SEQ ID NO: 897, the HCDR2 comprises the amino acid sequence of SEQ ID NO: 898, the HCDR3 comprises the amino acid sequence of SEQ ID NO: 899, the LCDR1 comprises the amino acid sequence of SEQ ID NO: 900, the LCDR2 comprises the amino acid sequence of SEQ ID NO: 901, and the LCDR3 comprises the amino acid sequence of SEQ ID NO: 902;
(12). the HCDR1 comprises the amino acid sequence of SEQ ID NO: 903, the HCDR2 comprises the amino acid sequence of SEQ ID NO: 904, the HCDR3 comprises the amino acid sequence of SEQ ID NO: 905, the LCDR1 comprises the amino acid sequence of SEQ ID NO: 906, the LCDR2 comprises the amino acid sequence of SEQ ID NO: 907, and the LCDR3 comprises the amino acid sequence of SEQ ID NO: 908;
(13). the HCDR1 comprises the amino acid sequence of SEQ ID NO: 909, the HCDR2 comprises the amino acid sequence of SEQ ID NO: 910, the HCDR3 comprises the amino acid sequence of SEQ ID NO: 911, the LCDR1 comprises the amino acid sequence of SEQ ID NO: 912, the LCDR2 comprises the amino acid sequence of SEQ ID NO: 913, and the LCDR3 comprises the amino acid sequence of SEQ ID NO: 914;
(14). the HCDR1 comprises the amino acid sequence of SEQ ID NO: 915, the HCDR2 comprises the amino acid sequence of SEQ ID NO: 916, the HCDR3 comprises the amino acid sequence of SEQ ID NO: 917, the LCDR1 comprises the amino acid sequence of SEQ ID NO: 918, the LCDR2 comprises the amino acid sequence of SEQ ID NO: 919, and the LCDR3 comprises the amino acid sequence of SEQ ID NO: 920;
(15). the HCDR1 comprises the amino acid sequence of SEQ ID NO: 921, the HCDR2 comprises the amino acid sequence of SEQ ID NO: 922, the HCDR3 comprises the amino acid sequence of SEQ ID NO: 923, the LCDR1 comprises the amino acid sequence of SEQ ID NO: 924, the LCDR2 comprises the amino acid sequence of SEQ ID NO: 925, and the LCDR3 comprises the amino acid sequence of SEQ ID NO: 926;
(16). the HCDR1 comprises the amino acid sequence of SEQ ID NO: 927, the HCDR2 comprises the amino acid sequence of SEQ ID NO: 928, the HCDR3 comprises the amino acid sequence of SEQ ID NO: 929, the LCDR1 comprises the amino acid sequence of SEQ ID NO: 930, the LCDR2 comprises the amino acid sequence of SEQ ID NO: 931, and the LCDR3 comprises the amino acid sequence of SEQ ID NO: 932;
(17). the HCDR1 comprises the amino acid sequence of SEQ ID NO: 933, the HCDR2 comprises the amino acid sequence of SEQ ID NO: 934, the HCDR3 comprises the amino acid sequence of SEQ ID NO: 935, the LCDR1 comprises the amino acid sequence of SEQ ID NO: 936, the LCDR2 comprises the amino acid sequence of SEQ ID NO: 937, and the LCDR3 comprises the amino acid sequence of SEQ ID NO: 938;
(18). the HCDR1 comprises the amino acid sequence of SEQ ID NO: 939, the HCDR2 comprises the amino acid sequence of SEQ ID NO: 940, the HCDR3 comprises the amino acid sequence of SEQ ID NO: 941, the LCDR1 comprises the amino acid sequence of SEQ ID NO: 942, the LCDR2 comprises the amino acid sequence of SEQ ID NO: 943, and the LCDR3 comprises the amino acid sequence of SEQ ID NO: 944;
(19). the HCDR1 comprises the amino acid sequence of SEQ ID NO: 945, the HCDR2 comprises the amino acid sequence of SEQ ID NO: 946, the HCDR3 comprises the amino acid sequence of SEQ ID NO: 947, the LCDR1 comprises the amino acid sequence of SEQ ID NO: 948, the LCDR2 comprises the amino acid sequence of SEQ ID NO: 949, and the LCDR3 comprises the amino acid sequence of SEQ ID NO: 950;
(20). the HCDR1 comprises the amino acid sequence of SEQ ID NO: 951, the HCDR2 comprises the amino acid sequence of SEQ ID NO: 952, the HCDR3 comprises the amino acid sequence of SEQ ID NO: 953, the LCDR1 comprises the amino acid sequence of SEQ ID NO: 954, the LCDR2 comprises the amino acid sequence of SEQ ID NO: 955, and the LCDR3 comprises the amino acid sequence of SEQ ID NO: 956;
(21). the HCDR1 comprises the amino acid sequence of SEQ ID NO: 957, the HCDR2 comprises the amino acid sequence of SEQ ID NO: 958, the HCDR3 comprises the amino acid sequence of SEQ ID NO: 959, the LCDR1 comprises the amino acid sequence of SEQ ID NO: 960, the LCDR2 comprises the amino acid sequence of SEQ ID NO: 961, and the LCDR3 comprises the amino acid sequence of SEQ ID NO: 962;
(22). the HCDR1 comprises the amino acid sequence of SEQ ID NO: 963, the HCDR2 comprises the amino acid sequence of SEQ ID NO: 964, the HCDR3 comprises the amino acid sequence of SEQ ID NO: 965, the LCDR1 comprises the amino acid sequence of SEQ ID NO: 966, the LCDR2 comprises the amino acid sequence of SEQ ID NO: 967, and the LCDR3 comprises the amino acid sequence of SEQ ID NO: 968;
(23). the HCDR1 comprises the amino acid sequence of SEQ ID NO: 969, the HCDR2 comprises the amino acid sequence of SEQ ID NO: 970, the HCDR3 comprises the amino acid sequence of SEQ ID NO: 971, the LCDR1 comprises the amino acid sequence of SEQ ID NO: 972, the LCDR2 comprises the amino acid sequence of SEQ ID NO: 973, and the LCDR3 comprises the amino acid sequence of SEQ ID NO: 974;
(24). the HCDR1 comprises the amino acid sequence of SEQ ID NO: 975, the HCDR2 comprises the amino acid sequence of SEQ ID NO: 976, the HCDR3 comprises the amino acid sequence of SEQ ID NO: 977, the LCDR1 comprises the amino acid sequence of SEQ ID NO: 978, the LCDR2 comprises the amino acid sequence of SEQ ID NO: 979, and the LCDR3 comprises the amino acid sequence of SEQ ID NO: 980;
(25). the HCDR1 comprises the amino acid sequence of SEQ ID NO: 981, the HCDR2 comprises the amino acid sequence of SEQ ID NO: 982, the HCDR3 comprises the amino acid sequence of SEQ ID NO: 983, the LCDR1 comprises the amino acid sequence of SEQ ID NO: 984, the LCDR2 comprises the amino acid sequence of SEQ ID NO: 985, and the LCDR3 comprises the amino acid sequence of SEQ ID NO: 986;
(26). the HCDR1 comprises the amino acid sequence of SEQ ID NO: 993, the HCDR2 comprises the amino acid sequence of SEQ ID NO: 994, the HCDR3 comprises the amino acid sequence of SEQ ID NO: 995, the LCDR1 comprises the amino acid sequence of SEQ ID NO: 996, the LCDR2 comprises the amino acid sequence of SEQ ID NO: 997, and the LCDR3 comprises the amino acid sequence of SEQ ID NO: 998;
(27). the HCDR1 comprises the amino acid sequence of SEQ ID NO: 999, the HCDR2 comprises the amino acid sequence of SEQ ID NO: 1000, the HCDR3 comprises the amino acid sequence of SEQ ID NO: 1001, the LCDR1 comprises the amino acid sequence of SEQ ID NO: 1002, the LCDR2 comprises the amino acid sequence of SEQ ID NO: 1003, and the LCDR3 comprises the amino acid sequence of SEQ ID NO: 1004;
(28). the HCDR1 comprises the amino acid sequence of SEQ ID NO: 1005, the HCDR2 comprises the amino acid sequence of SEQ ID NO: 1006, the HCDR3 comprises the amino acid sequence of SEQ ID NO: 1007, the LCDR1 comprises the amino acid sequence of SEQ ID NO: 1008, the LCDR2 comprises the amino acid sequence of SEQ ID NO: 1009, and the LCDR3 comprises the amino acid sequence of SEQ ID NO: 1010;
(29). the HCDR1 comprises the amino acid sequence of SEQ ID NO: 1011, the HCDR2 comprises the amino acid sequence of SEQ ID NO: 1012, the HCDR3 comprises the amino acid sequence of SEQ ID NO: 1013, the LCDR1 comprises the amino acid sequence of SEQ ID NO: 1014, the LCDR2 comprises the amino acid sequence of SEQ ID NO: 1015, and the LCDR3 comprises the amino acid sequence of SEQ ID NO: 1016;
(30). the HCDR1 comprises the amino acid sequence of SEQ ID NO: 1017, the HCDR2 comprises the amino acid sequence of SEQ ID NO: 1018, the HCDR3 comprises the amino acid sequence of SEQ ID NO: 1019, the LCDR1 comprises the amino acid sequence of SEQ ID NO: 1020, the LCDR2 comprises the amino acid sequence of SEQ ID NO: 1021, and the LCDR3 comprises the amino acid sequence of SEQ ID NO: 1022;
(31). the HCDR1 comprises the amino acid sequence of SEQ ID NO: 1023, the HCDR2 comprises the amino acid sequence of SEQ ID NO: 1024, the HCDR3 comprises the amino acid sequence of SEQ ID NO: 1025, the LCDR1 comprises the amino acid sequence of SEQ ID NO: 1026, the LCDR2 comprises the amino acid sequence of SEQ ID NO: 1027, and the LCDR3 comprises the amino acid sequence of SEQ ID NO: 1028;
(32). the HCDR1 comprises the amino acid sequence of SEQ ID NO: 1029, the HCDR2 comprises the amino acid sequence of SEQ ID NO: 1030, the HCDR3 comprises the amino acid sequence of SEQ ID NO: 1031, the LCDR1 comprises the amino acid sequence of SEQ ID NO: 1032, the LCDR2 comprises the amino acid sequence of SEQ ID NO: 1033, and the LCDR3 comprises the amino acid sequence of SEQ ID NO: 1034;
(33). the HCDR1 comprises the amino acid sequence of SEQ ID NO: 1035, the HCDR2 comprises the amino acid sequence of SEQ ID NO: 1036, the HCDR3 comprises the amino acid sequence of SEQ ID NO: 1037, the LCDR1 comprises the amino acid sequence of SEQ ID NO: 1038, the LCDR2 comprises the amino acid sequence of SEQ ID NO: 1039, and the LCDR3 comprises the amino acid sequence of SEQ ID NO: 1040;
(34). the HCDR1 comprises the amino acid sequence of SEQ ID NO: 1041, the HCDR2 comprises the amino acid sequence of SEQ ID NO: 1042, the HCDR3 comprises the amino acid sequence of SEQ ID NO: 1043, the LCDR1 comprises the amino acid sequence of SEQ ID NO: 1044, the LCDR2 comprises the amino acid sequence of SEQ ID NO: 1045, and the LCDR3 comprises the amino acid sequence of SEQ ID NO: 1046;
(35). the HCDR1 comprises the amino acid sequence of SEQ ID NO: 1047, the HCDR2 comprises the amino acid sequence of SEQ ID NO: 1048, the HCDR3 comprises the amino acid sequence of SEQ ID NO: 1049, the LCDR1 comprises the amino acid sequence of SEQ ID NO: 1050, the LCDR2 comprises the amino acid sequence of SEQ ID NO: 1051, and the LCDR3 comprises the amino acid sequence of SEQ ID NO: 1052;
(36). the HCDR1 comprises the amino acid sequence of SEQ ID NO: 1053, the HCDR2 comprises the amino acid sequence of SEQ ID NO: 1054, the HCDR3 comprises the amino acid sequence of SEQ ID NO: 1055, the LCDR1 comprises the amino acid sequence of SEQ ID NO: 1056, the LCDR2 comprises the amino acid sequence of SEQ ID NO: 1057, and the LCDR3 comprises the amino acid sequence of SEQ ID NO: 1058;
(37). the HCDR1 comprises the amino acid sequence of SEQ ID NO: 1059, the HCDR2 comprises the amino acid sequence of SEQ ID NO: 1060, the HCDR3 comprises the amino acid sequence of SEQ ID NO: 1061, the LCDR1 comprises the amino acid sequence of SEQ ID NO: 1062, the LCDR2 comprises the amino acid sequence of SEQ ID NO: 1063, and the LCDR3 comprises the amino acid sequence of SEQ ID NO: 1064;
(38). the HCDR1 comprises the amino acid sequence of SEQ ID NO: 1065, the HCDR2 comprises the amino acid sequence of SEQ ID NO: 1066, the HCDR3 comprises the amino acid sequence of SEQ ID NO: 1067, the LCDR1 comprises the amino acid sequence of SEQ ID NO: 1068, the LCDR2 comprises the amino acid sequence of SEQ ID NO: 1069, and the LCDR3 comprises the amino acid sequence of SEQ ID NO: 1070;
(39). the HCDR1 comprises the amino acid sequence of SEQ ID NO: 1071, the HCDR2 comprises the amino acid sequence of SEQ ID NO: 1072, the HCDR3 comprises the amino acid sequence of SEQ ID NO: 1073, the LCDR1 comprises the amino acid sequence of SEQ ID NO: 1074, the LCDR2 comprises the amino acid sequence of SEQ ID NO: 1075, and the LCDR3 comprises the amino acid sequence of SEQ ID NO: 1076;
(40). the HCDR1 comprises the amino acid sequence of SEQ ID NO: 1077, the HCDR2 comprises the amino acid sequence of SEQ ID NO: 1078, the HCDR3 comprises the amino acid sequence of SEQ ID NO: 1079, the LCDR1 comprises the amino acid sequence of SEQ ID NO: 1080, the LCDR2 comprises the amino acid sequence of SEQ ID NO: 1081, and the LCDR3 comprises the amino acid sequence of SEQ ID NO: 1082;
(41). the HCDR1 comprises the amino acid sequence of SEQ ID NO: 1083, the HCDR2 comprises the amino acid sequence of SEQ ID NO: 1084, the HCDR3 comprises the amino acid sequence of SEQ ID NO: 1085, the LCDR1 comprises the amino acid sequence of SEQ ID NO: 1086, the LCDR2 comprises the amino acid sequence of SEQ ID NO: 1087, and the LCDR3 comprises the amino acid sequence of SEQ ID NO: 1088;
(42). the HCDR1 comprises the amino acid sequence of SEQ ID NO: 1089, the HCDR2 comprises the amino acid sequence of SEQ ID NO: 1090, the HCDR3 comprises the amino acid sequence of SEQ ID NO: 1091, the LCDR1 comprises the amino acid sequence of SEQ ID NO: 1092, the LCDR2 comprises the amino acid sequence of SEQ ID NO: 1093, and the LCDR3 comprises the amino acid sequence of SEQ ID NO: 1094;
(43). the HCDR1 comprises the amino acid sequence of SEQ ID NO: 1095, the HCDR2 comprises the amino acid sequence of SEQ ID NO: 1096, the HCDR3 comprises the amino acid sequence of SEQ ID NO: 1097, the LCDR1 comprises the amino acid sequence of SEQ ID NO: 1098, the LCDR2 comprises the amino acid sequence of SEQ ID NO: 1099, and the LCDR3 comprises the amino acid sequence of SEQ ID NO: 1100;
(44). the HCDR1 comprises the amino acid sequence of SEQ ID NO: 1101, the HCDR2 comprises the amino acid sequence of SEQ ID NO: 1102, the HCDR3 comprises the amino acid sequence of SEQ ID NO: 1103, the LCDR1 comprises the amino acid sequence of SEQ ID NO: 1104, the LCDR2 comprises the amino acid sequence of SEQ ID NO: 1105, and the LCDR3 comprises the amino acid sequence of SEQ ID NO: 1106;
(45). the HCDR1 comprises the amino acid sequence of SEQ ID NO: 1107, the HCDR2 comprises the amino acid sequence of SEQ ID NO: 1108, the HCDR3 comprises the amino acid sequence of SEQ ID NO: 1109, the LCDR1 comprises the amino acid sequence of SEQ ID NO: 1110, the LCDR2 comprises the amino acid sequence of SEQ ID NO: 1111, and the LCDR3 comprises the amino acid sequence of SEQ ID NO: 1112;
(46). the HCDR1 comprises the amino acid sequence of SEQ ID NO: 1113, the HCDR2 comprises the amino acid sequence of SEQ ID NO: 1114, the HCDR3 comprises the amino acid sequence of SEQ ID NO: 1115, the LCDR1 comprises the amino acid sequence of SEQ ID NO: 1116, the LCDR2 comprises the amino acid sequence of SEQ ID NO: 1117, and the LCDR3 comprises the amino acid sequence of SEQ ID NO: 1118;
(47). the HCDR1 comprises the amino acid sequence of SEQ ID NO: 1119, the HCDR2 comprises the amino acid sequence of SEQ ID NO: 1120, the HCDR3 comprises the amino acid sequence of SEQ ID NO: 1121, the LCDR1 comprises the amino acid sequence of SEQ ID NO: 1122, the LCDR2 comprises the amino acid sequence of SEQ ID NO: 1123, and the LCDR3 comprises the amino acid sequence of SEQ ID NO: 1124; or
(48). the HCDR1 comprises the amino acid sequence of SEQ ID NO: 1125, the HCDR2 comprises the amino acid sequence of SEQ ID NO: 1126, the HCDR3 comprises the amino acid sequence of SEQ ID NO: 1127, the LCDR1 comprises the amino acid sequence of SEQ ID NO: 1128, the LCDR2 comprises the amino acid sequence of SEQ ID NO: 1129, and the LCDR3 comprises the amino acid sequence of SEQ ID NO: 1130.

6. The antibody or antigen binding fragment of claim 4 or 5, comprises:
(1). a VH comprising the amino acid sequence of SEQ ID NO: 1167 and a VL comprising the amino acid sequence of SEQ ID NO: 1168;
(2). a VH comprising the amino acid sequence of SEQ ID NO: 1169 and a VL comprising the amino acid sequence of SEQ ID NO: 1170;
(3). a VH comprising the amino acid sequence of SEQ ID NO: 1171 and a VL comprising the amino acid sequence of SEQ ID NO: 1172;
(4). a VH comprising the amino acid sequence of SEQ ID NO: 1173 and a VL comprising the amino acid sequence of SEQ ID NO: 1174;
(5). a VH comprising the amino acid sequence of SEQ ID NO: 1175 and a VL comprising the amino acid sequence of SEQ ID NO: 1176;
(6). a VH comprising the amino acid sequence of SEQ ID NO: 1177 and a VL comprising the amino acid sequence of SEQ ID NO: 1178.
(7). a VH comprising the amino acid sequence of SEQ ID NO: 401 and a VL comprising the amino acid sequence of SEQ ID NO: 402;
(8). a VH comprising the amino acid sequence of SEQ ID NO: 403 and a VL comprising the amino acid sequence of SEQ ID NO: 404;
(9). a VH comprising the amino acid sequence of SEQ ID NO: 405 and a VL comprising the amino acid sequence of SEQ ID NO: 406;
(10). a VH comprising the amino acid sequence of SEQ ID NO: 407 and a VL comprising the amino acid sequence of SEQ ID NO: 408;
(11). a VH comprising the amino acid sequence of SEQ ID NO: 409 and a VL comprising the amino acid sequence of SEQ ID NO: 410;
(12). a VH comprising the amino acid sequence of SEQ ID NO: 411 and a VL comprising the amino acid sequence of SEQ ID NO: 412;
(13). a VH comprising the amino acid sequence of SEQ ID NO: 413 and a VL comprising the amino acid sequence of SEQ ID NO: 414;
(14). a VH comprising the amino acid sequence of SEQ ID NO: 415 and a VL comprising the amino acid sequence of SEQ ID NO: 416;
(15). a VH comprising the amino acid sequence of SEQ ID NO: 417 and a VL comprising the amino acid sequence of SEQ ID NO: 418;
(16). a VH comprising the amino acid sequence of SEQ ID NO: 419 and a VL comprising the amino acid sequence of SEQ ID NO: 420;
(17). a VH comprising the amino acid sequence of SEQ ID NO: 421 and a VL comprising the amino acid sequence of SEQ ID NO: 422;
(18). a VH comprising the amino acid sequence of SEQ ID NO: 423 and a VL comprising the amino acid sequence of SEQ ID NO: 424;
(19). a VH comprising the amino acid sequence of SEQ ID NO: 425 and a VL comprising the amino acid sequence of SEQ ID NO: 426;
(20). a VH comprising the amino acid sequence of SEQ ID NO: 427 and a VL comprising the amino acid sequence of SEQ ID NO: 428;
(21). a VH comprising the amino acid sequence of SEQ ID NO: 429 and a VL comprising the amino acid sequence of SEQ ID NO: 430;
(22). a VH comprising the amino acid sequence of SEQ ID NO: 431 and a VL comprising the amino acid sequence of SEQ ID NO: 432;
(23). a VH comprising the amino acid sequence of SEQ ID NO: 433 and a VL comprising the amino acid sequence of SEQ ID NO: 434;
(24). a VH comprising the amino acid sequence of SEQ ID NO: 435 and a VL comprising the amino acid sequence of SEQ ID NO: 436;
(25). a VH comprising the amino acid sequence of SEQ ID NO: 437 and a VL comprising the amino acid sequence of SEQ ID NO: 438;
(26). a VH comprising the amino acid sequence of SEQ ID NO: 439 and a VL comprising the amino acid sequence of SEQ ID NO: 440;
(27). a VH comprising the amino acid sequence of SEQ ID NO: 441 and a VL comprising the amino acid sequence of SEQ ID NO: 442;
(28). a VH comprising the amino acid sequence of SEQ ID NO: 443 and a VL comprising the amino acid sequence of SEQ ID NO: 444;
(29). a VH comprising the amino acid sequence of SEQ ID NO: 445 and a VL comprising the amino acid sequence of SEQ ID NO: 446;
(30). a VH comprising the amino acid sequence of SEQ ID NO: 447 and a VL comprising the amino acid sequence of SEQ ID NO: 448;
(31). a VH comprising the amino acid sequence of SEQ ID NO: 449 and a VL comprising the amino acid sequence of SEQ ID NO: 450;
(32). a VH comprising the amino acid sequence of SEQ ID NO: 451 and a VL comprising the amino acid sequence of SEQ ID NO: 452;
(33). a VH comprising the amino acid sequence of SEQ ID NO: 453 and a VL comprising the amino acid sequence of SEQ ID NO: 454;
(34). a VH comprising the amino acid sequence of SEQ ID NO: 455 and a VL comprising the amino acid sequence of SEQ ID NO: 456;
(35). a VH comprising the amino acid sequence of SEQ ID NO: 457 and a VL comprising the amino acid sequence of SEQ ID NO: 458;
(36). a VH comprising the amino acid sequence of SEQ ID NO: 459 and a VL comprising the amino acid sequence of SEQ ID NO: 460;
(37). a VH comprising the amino acid sequence of SEQ ID NO: 461 and a VL comprising the amino acid sequence of SEQ ID NO: 462;
(38). a VH comprising the amino acid sequence of SEQ ID NO: 463 and a VL comprising the amino acid sequence of SEQ ID NO: 464;
(39). a VH comprising the amino acid sequence of SEQ ID NO: 465 and a VL comprising the amino acid sequence of SEQ ID NO: 466;
(40). a VH comprising the amino acid sequence of SEQ ID NO: 467 and a VL comprising the amino acid sequence of SEQ ID NO: 468;
(41). a VH comprising the amino acid sequence of SEQ ID NO: 469 and a VL comprising the amino acid sequence of SEQ ID NO: 470;
(42). a VH comprising the amino acid sequence of SEQ ID NO: 471 and a VL comprising the amino acid sequence of SEQ ID NO: 472;
(43). a VH comprising the amino acid sequence of SEQ ID NO: 473 and a VL comprising the amino acid sequence of SEQ ID NO: 474;
(44). a VH comprising the amino acid sequence of SEQ ID NO: 475 and a VL comprising the amino acid sequence of SEQ ID NO: 476;
(45). a VH comprising the amino acid sequence of SEQ ID NO: 477 and a VL comprising the amino acid sequence of SEQ ID NO: 478;
(46). a VH comprising the amino acid sequence of SEQ ID NO: 479 and a VL comprising the amino acid sequence of SEQ ID NO: 480;
(47). a VH comprising the amino acid sequence of SEQ ID NO: 481 and a VL comprising the amino acid sequence of SEQ ID NO: 482;
(48). a VH comprising the amino acid sequence of SEQ ID NO: 483 and a VL comprising the amino acid sequence of SEQ ID NO: 484;
(49). a VH comprising the amino acid sequence of SEQ ID NO: 485 and a VL comprising the amino acid sequence of SEQ ID NO: 486;
(50). a VH comprising the amino acid sequence of SEQ ID NO: 487 and a VL comprising the amino acid sequence of SEQ ID NO: 488;
(51). a VH comprising the amino acid sequence of SEQ ID NO: 489 and a VL comprising the amino acid sequence of SEQ ID NO: 490;
(52). a VH comprising the amino acid sequence of SEQ ID NO: 491 and a VL comprising the amino acid sequence of SEQ ID NO: 492; or
(53). a VH comprising the amino acid sequence of SEQ ID NO: 493 and a VL comprising the amino acid sequence of SEQ ID NO: 494.

7. A multispecific antibody or fragment thereof, comprising a first binding domain that binds to LILRB1 and a second binding domain that binds to LILRB2, wherein the first binding domain comprises an antibody or antigen binding fragment thereof according to claims 1 to 3, and wherein optionally the second binding domain comprises an antibody or antigen binding fragment thereof according to claims 4 to 6.

8. A multispecific antibody or fragment thereof, comprising a first binding domain that binds to LILRB1 and a second binding domain that binds to LILRB2, wherein the second binding domain comprises an antibody or antigen binding fragment thereof according to claims 4 to 6, and wherein optionally the first binding domain comprises an antibody or antigen binding fragment thereof according to claims 1 to 3.

9. The multispecific antibody or fragment thereof of claim 7 or 8, wherein the multispecific antibody or fragment thereof has an antibody format of immunoglobulin-single chain antibody fragment (IgG-ScFv).

10. The multispecific antibody or fragment thereof of claim 7 or 8, wherein the multispecific antibody or fragment thereof has an antibody format of dual-variable-domain-immunoglobulin (DVD-Ig).

11. The multispecific antibody or fragment thereof of claim 7 or 8,
wherein the first binding domain comprises any one or more of (1)-(6):
(1). a HCDR1, a HCDR2, and a HCDR3 as set forth in a VH comprising the amino acid sequence of SEQ ID NO: 1155 and a LCDR1, a LCDR2, and a LCDR3 as set forth in a VL comprising the amino acid sequence of SEQ ID NO: 1156;
(2). a HCDR1, a HCDR2, and a HCDR3 as set forth in a VH comprising the amino acid sequence of SEQ ID NO: 1157 and a LCDR1, a LCDR2, and a LCDR3 as set forth in a VL comprising the amino acid sequence of SEQ ID NO: 1158;
(3). a HCDR1, a HCDR2, and a HCDR3 as set forth in a VH comprising the amino acid sequence of SEQ ID NO: 1159 and a LCDR1, a LCDR2, and a LCDR3 as set forth in a VL comprising the amino acid sequence of SEQ ID NO: 1160;
(4). a HCDR1, a HCDR2, and a HCDR3 as set forth in a VH comprising the amino acid sequence of SEQ ID NO: 1161 and a LCDR1, a LCDR2, and a LCDR3 as set forth in a VL comprising the amino acid sequence of SEQ ID NO: 1162;
(5). a HCDR1, a HCDR2, and a HCDR3 as set forth in a VH comprising the amino acid sequence of SEQ ID NO: 1163 and a LCDR1, a LCDR2, and a LCDR3 as set forth in a VL comprising the amino acid sequence of SEQ ID NO: 1164; or
(6). a HCDR1, a HCDR2, and a HCDR3 as set forth in a VH comprising the amino acid sequence of SEQ ID NO: 1165 and a LCDR1, a LCDR2, and a LCDR3 as set forth in a VL comprising the amino acid sequence of SEQ ID NO: 1166; and
wherein the second binding domain comprises any one or more of (1)-(6):
(1). a HCDR1, a HCDR2, and a HCDR3 as set forth in a VH comprising the amino acid sequence of SEQ ID NO: 1167 and a LCDR1, a LCDR2, and a LCDR3 as set forth in a VL comprising the amino acid sequence of SEQ ID NO: 1168;
(2). a HCDR1, a HCDR2, and a HCDR3 as set forth in a VH comprising the amino acid sequence of SEQ ID NO: 1169 and a LCDR1, a LCDR2, and a LCDR3 as set forth in a VL comprising the amino acid sequence of SEQ ID NO: 1170;
(3). a HCDR1, a HCDR2, and a HCDR3 as set forth in a VH comprising the amino acid sequence of SEQ ID NO: 1171 and a LCDR1, a LCDR2, and a LCDR3 as set forth in a VL comprising the amino acid sequence of SEQ ID NO: 1172;
(4). a HCDR1, a HCDR2, and a HCDR3 as set forth in a VH comprising the amino acid sequence of SEQ ID NO: 1173 and a LCDR1, a LCDR2, and a LCDR3 as set forth in a VL comprising the amino acid sequence of SEQ ID NO: 1174;
(5). a HCDR1, a HCDR2, and a HCDR3 as set forth in a VH comprising the amino acid sequence of SEQ ID NO: 1175 and a LCDR1, a LCDR2, and a LCDR3 as set forth in a VL comprising the amino acid sequence of SEQ ID NO: 1176; or
(6). a HCDR1, a HCDR2, and a HCDR3 as set forth in a VH comprising the amino acid sequence of SEQ ID NO: 1177 and a LCDR1, a LCDR2, and a LCDR3 as set forth in a VL comprising the amino acid sequence of SEQ ID NO: 1178.

12. The multispecific antibody or fragment thereof of claim 11,
wherein the first binding domain comprises any one or more of (1)-(6):
(1). a VH comprising the amino acid sequence of SEQ ID NO: 1155 and a VL comprising the amino acid sequence of SEQ ID NO: 1156;
(2). a VH comprising the amino acid sequence of SEQ ID NO: 1157 and a VL comprising the amino acid sequence of SEQ ID NO: 1158;
(3). a VH comprising the amino acid sequence of SEQ ID NO: 1159 and a VL comprising the amino acid sequence of SEQ ID NO: 1160;
(4). a VH comprising the amino acid sequence of SEQ ID NO: 1161 and a VL comprising the amino acid sequence of SEQ ID NO: 1162;
(5). a VH comprising the amino acid sequence of SEQ ID NO: 1163 and a VL comprising the amino acid sequence of SEQ ID NO: 1164; or
(6). a VH comprising the amino acid sequence of SEQ ID NO: 1165 and a VL comprising the amino acid sequence of SEQ ID NO: 1166; and
wherein the second binding domain comprises any one or more of (1)-(6):
(1). a VH comprising the amino acid sequence of SEQ ID NO: 1167 and a VL comprising the amino acid sequence of SEQ ID NO: 1168;
(2). a VH comprising the amino acid sequence of SEQ ID NO: 1169 and a VL comprising the amino acid sequence of SEQ ID NO: 1170;
(3). a VH comprising the amino acid sequence of SEQ ID NO: 1171 and a VL comprising the amino acid sequence of SEQ ID NO: 1172;
(4). a VH comprising the amino acid sequence of SEQ ID NO: 1173 and a VL comprising the amino acid sequence of SEQ ID NO: 1174;
(5). a VH comprising the amino acid sequence of SEQ ID NO: 1175 and a VL comprising the amino acid sequence of SEQ ID NO: 1176; or
(6) a VH comprising the amino acid sequence of SEQ ID NO: 1177 and a VL comprising the amino acid sequence of SEQ ID NO: 1178.

13. The multispecific antibody or fragment thereof of claim 11 or 12, wherein the multispecific antibody or fragment thereof comprises:
(1). a first polypeptide comprising the amino acid sequence of SEQ ID NO: 1180, and a second polypeptide comprising the amino acid sequence of SEQ ID NO: 1181;
(2). a first polypeptide comprising the amino acid sequence of SEQ ID NO: 1182, and a second polypeptide comprising the amino acid sequence of SEQ ID NO: 1183;
(3). a first polypeptide comprising the amino acid sequence of SEQ ID NO: 1184, and a second polypeptide comprising the amino acid sequence of SEQ ID NO: 1185;
(4). a first polypeptide comprising the amino acid sequence of SEQ ID NO: 1186, and a second polypeptide comprising the amino acid sequence of SEQ ID NO: 1187;
(5). a first polypeptide comprising the amino acid sequence of SEQ ID NO: 1188, and a second polypeptide comprising the amino acid sequence of SEQ ID NO: 1189;
(6). a first polypeptide comprising the amino acid sequence of SEQ ID NO: 1190, and a second polypeptide comprising the amino acid sequence of SEQ ID NO: 1191;
(7). a first polypeptide comprising the amino acid sequence of SEQ ID NO: 1192, and a second polypeptide comprising the amino acid sequence of SEQ ID NO: 1193;
(8). a first polypeptide comprising the amino acid sequence of SEQ ID NO: 1194, and a second polypeptide comprising the amino acid sequence of SEQ ID NO: 1195;
(9). a first polypeptide comprising the amino acid sequence of SEQ ID NO: 1196, and a second polypeptide comprising the amino acid sequence of SEQ ID NO: 1197;
(10). a first polypeptide comprising the amino acid sequence of SEQ ID NO: 1198, and a second polypeptide comprising the amino acid sequence of SEQ ID NO: 1199;
(11). a first polypeptide comprising the amino acid sequence of SEQ ID NO: 1200, and a second polypeptide comprising the amino acid sequence of SEQ ID NO: 1201; or
(12). a first polypeptide comprising the amino acid sequence of SEQ ID NO: 1202, and a second polypeptide comprising the amino acid sequence of SEQ ID NO: 1203.

14. The antibody or antigen binding fragment of any one of claims 1-13, wherein the antibody is an IgG.

15. A nucleic acid molecule encoding the antibody or antigen binding fragment of any one of claims 1-14.

16. A vector comprising the nucleic acid molecule of claim 15.

17. A host cell transformed with the vector of claim 16.

18. A composition comprising a therapeutically effective amount of the antibody or antigen binding fragment of any one of claims 1-14, the nucleic acid molecule of claim 15, or the vector of claim 16, and a pharmaceutically acceptable excipient.

19. A method of preventing suppression of an immune cell or activating a response mediated by an immune cell, comprising contacting the immune cell with the antibody or fragment thereof of any one of claims 1-14 or the pharmaceutical composition of claim 18.

20. The method of claim 19, wherein the immune cell is an NK cell, a macrophage, a T cell or a dendric cell.

21. The method of claim 20, wherein the T cell is a CD8+ T cell.

22. The method of any one of claims 19-21, wherein the immune cell expresses LILRB1 and/or LILRB2.

23. The method of any one of claims 19-22, wherein the response mediated by the immune cell is an anti-tumor response, optionally wherein the tumor cell expresses HLA-A2, HLA-G, and/or ANGPTLs.

24. A method for treating a disease or disorder in a subject comprising administering to the subject the antibody or antigen binding fragment of any one of claims 1-14, or the pharmaceutical composition of claim 18.

25. The method of claim 24, wherein the disease or disorder is a cancer, optionally the cancer expresses HLA-A2, HLA-G and/or ANGPTLs.

26. The method of claim 24, wherein the disease or disorder is a solid tumor or a blood tumor.

27. The method of any one of claims 24-26, wherein the subject is a human subject.

28. The method of claim 24, wherein the antibody or antigen binding fragment is used as part of a combination therapy.

29. The method of claim 28, wherein the antibody or antigen binding fragment is used in combination with a PD-1 inhibitor, wherein optionally, the PD-1 inhibitor is an anti-PD-1 antibody, wherein optionally the anti-PD-1 antibody is Nivolumab.

30. The method of claim 28, wherein the antibody or antigen binding fragment is used in combination with a PD-L1 inhibitor, wherein optionally, the PD-L1 inhibitor is an anti-PD-L1 antibody, wherein optionally the anti-PD-L1 antibody is LAE005.

31. The method of claim 28, wherein the antibody or antigen binding fragment is used in combination with an anti-CD47 antibody, wherein optionally the anti-CD47 antibody is Hu5F9-G4.

32. The method of claim 28, wherein the antibody or antigen binding fragment is used in combination with an anti-CD3 antibody, wherein optionally the anti-CD3 antibody is OKT3.


SEQUENCE LISTING (1285 sequences)

SEQ ID NO: 1 - protein, 449 aa, synthetic construct
  source 1..449: note = Antibody 12D12 Heavy Chain
QVQLQQPGAELVKPGASVRMSCKASGYTFTSYWVHWVKQRPGQGLEWIGVIDPSDSYTSYNQNFKGKATLTVDTSSKTAYIHLSSLTSEDSAVYFCARGERYDGDYFAMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 2 - protein, 214 aa, synthetic construct
  source 1..214: note = Antibody 12D12 Light Chain
DIVMTQSPASLSVSVGETVTITCRASENIYSNLAWYQQKQGKSPQLLVYAATNLADGVPSRFSGSRSGTQYSLKINSLQSEDFGTYYCQHFWNTPRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 446 aa, synthetic construct
  source 1..446: note = Antibody 15G8 Heavy Chain
DVQLQGSGPGLVKPSETLSLTCSVTGYSITSGYYWNWIRQFPGKKLEWMGYISYDGSNNYNPSLKNRITISRDTSKNQFSLKLNSVTAADTATYYCAHGYSYYYAMDAWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 4 - protein, 213 aa, synthetic construct
  source 1..213: note = Antibody 15G8 Light Chain
DIQMTQSPSSLSASVGDRVTITCRTSQDISNYLNWYQQKPGKAVKLLISYTSRLHSGVPSRFSGSGSGTDYTLTISSLQPEDFATYYCQQGNTLPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 444 aa, synthetic construct
  source 1..444: note = Antibody 3C1 Heavy Chain
EVQLVESGGGLVQPGGSLRLSCAASGFTFSDYDMHWVRQATGKGLEWVATIDTAGDTYYPDSVRGRFTISRENAKNSLSLQMNSLRAGDTAVYFCARDVPGTGFDHWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 6 - protein, 218 aa, synthetic construct
  source 1..218: note = Antibody 3C1 Light Chain
SSELTQDPAVSVALGQTVRITCQGDSLRSYYESWYQQKPGQAPVLVIYGKNNRPSGIPDRFSGSISGNTASLTITGTQAEDEADYYCNSRDNSGDHWVFGGGTRLTVLGQPRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 448 aa, synthetic construct
  source 1..448: note = Antibody p1E1 Heavy Chain
EVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARLPTRWVTTRYFDLWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 8 - protein, 221 aa, synthetic construct
  source 1..221: note = Antibody p1E1 Light Chain
ESVLTQPPSVSGAPGQRVTISCTGSSSNIGAGYDVHWYQQLPGTAPKLLIYGNSNRPSGVPDRFSVSKSGASASLAITGLQAEDEADYYCQSFDNSLSAYVFGGGTQLTVLGQPRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 450 aa, synthetic construct
  source 1..450: note = Antibody J19.h1 Heavy Chain
QITLKESGPTLVKPTQTLTLTCTFSGFSLNTYAMGVSWIRQPPGKALEWLASIWWNGNKYNNPSLKSRLTVTKDTSKNQVVLTMTNMDPVDTATYYCAHSRIIRFTDYVMDAWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 214 aa, synthetic construct
  source 1..214: note = Antibody J19.h1 Light Chain
DIQMTQSPSSLSTSVGDRVTITCRASEDIYNDLAWYQQKPGKAPKLLIYNANSLHTGVASRFSGSGSGTDFTFTISSLQPEDVATYFCQQYYDYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 452 aa, synthetic construct
  source 1..452: note = Antibody B2-19 Heavy Chain
QVQLVQSGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTTDRYSSSWYSPAFDIWGQGTTVSVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 216 aa, synthetic construct
  source 1..216: note = Antibody B2-19 Light Chain
QPVLTQPPSVSEAPRQRVTISCSGSSSNIGNNVVNWYQQLPGKAPKLLIYYDDLLPSGVSDRFSGSKSGTSASLAISGLQSEDEADYYCATWDDSLNGPVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 13 - protein, 452 aa, synthetic construct
  source 1..452: note = Antibody hz73D1.v1 Heavy Chain
QVQLVQSGAEVKKPGASVKVSCKASGYTFTDYYINWVRQAPGQGLEWMGNVNPNDGGTTYNQKFKGRVTMTTDTSTSTAYMELRSLRSDDTAVYYCARREIYFYGTIYYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 14 - protein, 218 aa, synthetic construct
  source 1..218: note = Antibody hz73D1.v1 Light Chain
DIQLTQSPSFLSASVGDRVTITCRASESVDYYGNSFMYWYQQKPGKAPKLLIYFASNLESGVPSRFSGSGSGTEFTLTISSLQPEDFATYYCQQNNEDPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 122 aa, synthetic construct
  source 1..122: note = Antibody 12D12 VH
QVQLQQPGAELVKPGASVRMSCKASGYTFTSYWVHWVKQRPGQGLEWIGVIDPSDSYTSYNQNFKGKATLTVDTSSKTAYIHLSSLTSEDSAVYFCARGERYDGDYFAMDYWGQGTSVTVSS

SEQ ID NO: 16 - protein, 107 aa, synthetic construct
  source 1..107: note = Antibody 12D12 VL
DIVMTQSPASLSVSVGETVTITCRASENIYSNLAWYQQKQGKSPQLLVYAATNLADGVPSRFSGSRSGTQYSLKINSLQSEDFGTYYCQHFWNTPRTFGGGTKLEIK

SEQ ID NO: 17 - protein, 119 aa, synthetic construct
  source 1..119: note = Antibody 15G8 VH
DVQLQGSGPGLVKPSETLSLTCSVTGYSITSGYYWNWIRQFPGKKLEWMGYISYDGSNNYNPSLKNRITISRDTSKNQFSLKLNSVTAADTATYYCAHGYSYYYAMDAWGQGTSVTVSS

SEQ ID NO: 18 - protein, 106 aa, synthetic construct
  source 1..106: note = Antibody 15G8 VL
DIQMTQSPSSLSASVGDRVTITCRTSQDISNYLNWYQQKPGKAVKLLISYTSRLHSGVPSRFSGSGSGTDYTLTISSLQPEDFATYYCQQGNTLPTFGQGTKLEIK

SEQ ID NO: 19 - protein, 117 aa, synthetic construct
  source 1..117: note = Antibody 3C1 VH
EVQLVESGGGLVQPGGSLRLSCAASGFTFSDYDMHWVRQATGKGLEWVATIDTAGDTYYPDSVRGRFTISRENAKNSLSLQMNSLRAGDTAVYFCARDVPGTGFDHWGQGTLVTVSS

SEQ ID NO: 20 - protein, 111 aa, synthetic construct
  source 1..111: note = Antibody 3C1 VL
SSELTQDPAVSVALGQTVRITCQGDSLRSYYESWYQQKPGQAPVLVIYGKNNRPSGIPDRFSGSISGNTASLTITGTQAEDEADYYCNSRDNSGDHWVFGGGTRLTVLGQP

SEQ ID NO: 21 - protein, 121 aa, synthetic construct
  source 1..121: note = Antibody p1E1 VH
EVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARLPTRWVTTRYFDLWGRGTLVTVSS

SEQ ID NO: 22 - protein, 114 aa, synthetic construct
  source 1..114: note = Antibody p1E1 VL
ESVLTQPPSVSGAPGQRVTISCTGSSSNIGAGYDVHWYQQLPGTAPKLLIYGNSNRPSGVPDRFSVSKSGASASLAITGLQAEDEADYYCQSFDNSLSAYVFGGGTQLTVLGQP

SEQ ID NO: 23 - protein, 123 aa, synthetic construct
  source 1..123: note = Antibody J19.h1 VH
QITLKESGPTLVKPTQTLTLTCTFSGFSLNTYAMGVSWIRQPPGKALEWLASIWWNGNKYNNPSLKSRLTVTKDTSKNQVVLTMTNMDPVDTATYYCAHSRIIRFTDYVMDAWGQGTLVTVSS

SEQ ID NO: 24 - protein, 107 aa, synthetic construct
  source 1..107: note = Antibody J19.h1 VL
DIQMTQSPSSLSTSVGDRVTITCRASEDIYNDLAWYQQKPGKAPKLLIYNANSLHTGVASRFSGSGSGTDFTFTISSLQPEDVATYFCQQYYDYPLTFGQGTKLEIK

SEQ ID NO: 25 - protein, 125 aa, synthetic construct
  source 1..125: note = Antibody B2-19 VH
QVQLVQSGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTTDRYSSSWYSPAFDIWGQGTTVSVSS

SEQ ID NO: 26 - protein, 110 aa, synthetic construct
  source 1..110: note = Antibody B2-19 VL
QPVLTQPPSVSEAPRQRVTISCSGSSSNIGNNVVNWYQQLPGKAPKLLIYYDDLLPSGVSDRFSGSKSGTSASLAISGLQSEDEADYYCATWDDSLNGPVFGGGTKLTVL

SEQ ID NO: 27 - protein, 125 aa, synthetic construct
  source 1..125: note = Antibody hz73D1.v1 VH
QVQLVQSGAEVKKPGASVKVSCKASGYTFTDYYINWVRQAPGQGLEWMGNVNPNDGGTTYNQKFKGRVTMTTDTSTSTAYMELRSLRSDDTAVYYCARREIYFYGTIYYYAMDYWGQGTLVTVSS

SEQ ID NO: 28 - protein, 111 aa, synthetic construct
  source 1..111: note = Antibody hz73D1.v1 VL
DIQLTQSPSFLSASVGDRVTITCRASESVDYYGNSFMYWYQQKPGKAPKLLIYFASNLESGVPSRFSGSGSGTEFTLTISSLQPEDFATYYCQQNNEDPWTFGGGTKVEIK

SEQ ID NO: 29 - protein, 5 aa, synthetic construct
  source 1..5: note = Antibody 12D12 CDR-H1
SYWVH

SEQ ID NO: 30 - protein, 17 aa, synthetic construct
  source 1..17: note = Antibody 12D12 CDR-H2
VIDPSDSYTSYNQNFKG

SEQ ID NO: 31 - protein, 13 aa, synthetic construct
  source 1..13: note = Antibody 12D12 CDR-H3
GERYDGDYFAMDY

SEQ ID NO: 32 - protein, 11 aa, synthetic construct
  source 1..11: note = Antibody 12D12 CDR-L1
RASENIYSNLA

SEQ ID NO: 33 - protein, 7 aa, synthetic construct
  source 1..7: note = Antibody 12D12 CDR-L2
AATNLAD

SEQ ID NO: 34 - protein, 9 aa, synthetic construct
  source 1..9: note = Antibody 12D12 CDR-L3
QHFWNTPRT

SEQ ID NO: 35 - protein, 6 aa, synthetic construct
  source 1..6: note = Antibody 15G8 CDR-H1
SGYYWN

SEQ ID NO: 36 - protein, 16 aa, synthetic construct
  source 1..16: note = Antibody 15G8 CDR-H2
YISYDGSNNYNPSLKN

SEQ ID NO: 37 - protein, 10 aa, synthetic construct
  source 1..10: note = Antibody 15G8 CDR-H3
GYSYYYAMDA

SEQ ID NO: 38 - protein, 11 aa, synthetic construct
  source 1..11: note = Antibody 15G8 CDR-L1
RTSQDISNYLN

SEQ ID NO: 39 - protein, 7 aa, synthetic construct
  source 1..7: note = Antibody 15G8 CDR-L2
YTSRLHS

SEQ ID NO: 40 - protein, 8 aa, synthetic construct
  source 1..8: note = Antibody 15G8 CDR-L3
QQGNTLPT

SEQ ID NO: 41 - protein, 5 aa, synthetic construct
  source 1..5: note = Antibody 3C1 CDR-H1
DYDMH

SEQ ID NO: 42 - protein, 16 aa, synthetic construct
  source 1..16: note = Antibody 3C1 CDR-H2
TIDTAGDTYYPDSVRG

SEQ ID NO: 43 - protein, 9 aa, synthetic construct
  source 1..9: note = Antibody 3C1 CDR-H3
DVPGTGFDH

SEQ ID NO: 44 - protein, 11 aa, synthetic construct
  source 1..11: note = Antibody 3C1 CDR-L1
QGDSLRSYYES

SEQ ID NO: 45 - protein, 7 aa, synthetic construct
  source 1..7: note = Antibody 3C1 CDR-L2
GKNNRPS

SEQ ID NO: 46 - protein, 11 aa, synthetic construct
  source 1..11: note = Antibody 3C1 CDR-L3
NSRDNSGDHWV

SEQ ID NO: 47 - protein, 5 aa, synthetic construct
  source 1..5: note = Antibody p1E1 CDR-H1
GYYWS

SEQ ID NO: 48 - protein, 16 aa, synthetic construct
  source 1..16: note = Antibody p1E1 CDR-H2
EINHSGSTNYNPSLKS

SEQ ID NO: 49 - protein, 13 aa, synthetic construct
  source 1..13: note = Antibody p1E1 CDR-H3
LPTRWVTTRYFDL

SEQ ID NO: 50 - protein, 14 aa, synthetic construct
  source 1..14: note = Antibody p1E1 CDR-L1
TGSSSNIGAGYDVH

SEQ ID NO: 51 - protein, 7 aa, synthetic construct
  source 1..7: note = Antibody p1E1 CDR-L2
GNSNRPS

SEQ ID NO: 52 - protein, 11 aa, synthetic construct
  source 1..11: note = Antibody p1E1 CDR-L3
QSFDNSLSAYV

SEQ ID NO: 53 - protein, 7 aa, synthetic construct
  source 1..7: note = Antibody J19.h1 CDR-H1
TYAMGVS

SEQ ID NO: 54 - protein, 16 aa, synthetic construct
  source 1..16: note = Antibody J19.h1 CDR-H2
SIWWNGNKYNNPSLKS

SEQ ID NO: 55 - protein, 13 aa, synthetic construct
  source 1..13: note = Antibody J19.h1 CDR-H3
SRIIRFTDYVMDA

SEQ ID NO: 56 - protein, 11 aa, synthetic construct
  source 1..11: note = Antibody J19.h1 CDR-L1
RASEDIYNDLA

SEQ ID NO: 57 - protein, 7 aa, synthetic construct
  source 1..7: note = Antibody J19.h1 CDR-L2
NANSLHT

SEQ ID NO: 58 - protein, 9 aa, synthetic construct
  source 1..9: note = Antibody J19.h1 CDR-L3
QQYYDYPLT

SEQ ID NO: 59 - protein, 5 aa, synthetic construct
  source 1..5: note = Antibody B2-19 CDR-H1
NAWMS

SEQ ID NO: 60 - protein, 19 aa, synthetic construct
  source 1..19: note = Antibody B2-19 CDR-H2
RIKSKTDGGTTDYAAPVKG

SEQ ID NO: 61 - protein, 14 aa, synthetic construct
  source 1..14: note = Antibody B2-19 CDR-H3
DRYSSSWYSPAFDI

SEQ ID NO: 62 - protein, 13 aa, synthetic construct
  source 1..13: note = Antibody B2-19 CDR-L1
SGSSSNIGNNVVN

SEQ ID NO: 63 - protein, 7 aa, synthetic construct
  source 1..7: note = Antibody B2-19 CDR-L2
YDDLLPS

SEQ ID NO: 64 - protein, 11 aa, synthetic construct
  source 1..11: note = Antibody B2-19 CDR-L3
ATWDDSLNGPV

SEQ ID NO: 65 - protein, 5 aa, synthetic construct
  source 1..5: note = Antibody hz73D1.v1 CDR-H1
DYYIN

SEQ ID NO: 66 - protein, 17 aa, synthetic construct
  source 1..17: note = Antibody hz73D1.v1 CDR-H2
NVNPNDGGTTYNQKFKG

SEQ ID NO: 67 - protein, 16 aa, synthetic construct
  source 1..16: note = Antibody hz73D1.v1 CDR-H3
REIYFYGTIYYYAMDY

SEQ ID NO: 68 - protein, 15 aa, synthetic construct
  source 1..15: note = Antibody hz73D1.v1 CDR-L1
RASESVDYYGNSFMY

SEQ ID NO: 69 - protein, 7 aa, synthetic construct
  source 1..7: note = Antibody hz73D1.v1 CDR-L2
FASNLES

SEQ ID NO: 70 - protein, 9 aa, synthetic construct
  source 1..9: note = Antibody hz73D1.v1 CDR-L3
QQNNEDPWT

SEQ ID NO: 71 - protein, 449 aa, synthetic construct
  source 1..449: note = Antibody LK-RB1-1 Heavy Chain
QIQLVQSGPELKKPGETVKISCKASGYSFTNFGMSWVKQAPGKALKWMGWINTYSGVPTYADDFKGRFAFSLETSANTAYLQINNLKNEDSATYFCARRRRTFDGYLCFDYWGQGATLTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 72 - protein, 222 aa, synthetic construct
  source 1..222: note = Antibody LK-RB1-1 Light Chain
QLVLTQSSSASFSLGASAKLTCTLSSQHSTYSIDWYQQQPLKPPKYVMELKTDGSHRTGDGIPDRFSGSSSGADRYLSISNIQPEDEAIYICGVGDTIKEQFVYVFGGGTKVTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 73 - protein, 449 aa, synthetic construct
  source 1..449: note = Antibody LK-RB1-2 Heavy Chain
QIQLVQSGPELKKPGETVKISCKASGYTFTTYGVSWVKQAPGKGLKWMGWINTYSGVSTYSDDFKGRFAFSLETSASTAYLQIINLKNEDAATYFCSRRRRTYDGYLSFDYWGQGTTLTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 74 - protein, 214 aa, synthetic construct
  source 1..214: note = Antibody LK-RB1-2 Light Chain
DIQMTQSPGSLSASVGETVTITCGASENIYGALNWFQRKPGKSPQLLIYGATNLADGMSSRFSGSGSGRQYSLQISSLRPDDVATYYCQNVLTTPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 75 - protein, 446 aa, synthetic construct
  source 1..446: note = Antibody LK-RB1-3 Heavy Chain
QVQLQQPGAELVKPGTSVNLSCKASGYTFTSYWIHWVKQRPGRGLEWIGRINPYSSGTRYNEKFKNKATLTVDKPSSTAYMQLTSLTSEDSAVYYCARFDGAPDYFDFWGQGTTLTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 76 - protein, 214 aa, synthetic construct
  source 1..214: note = Antibody LK-RB1-3 Light Chain
DIVMTQSQKFMPTSVGDRVNVTCKASQNVGINVAWYQQKLGQSPKALIYSASYRYSGVPDRFTGSGSGTDFTLTINSVQSEDLADYFCQQYNNYPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 77 - protein, 446 aa, synthetic construct
  source 1..446: note = Antibody LK-RB1-4 Heavy Chain
QVQLQQPGAELVKPGASVKLPCKTSGYTFTSYWMHWVKQRPGRGLEWIGRIDPHYSGTKYNEKFKNKATMTVDTPSSTAYMQLSSLTSEDSAVYYCARFDGDPYYFDYWGPGTTLTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 78 - protein, 214 aa, synthetic construct
  source 1..214: note = Antibody LK-RB1-4 Light Chain
DIVMTQSQKFMSTSVGDRVSVTCKASQNVGTNVAWYKQKAGQSPKAVIYSASYRYSGVPDRFTGSGSGTDFTLSISNVQSEDLAEYFCQQYNNNPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 79 - protein, 447 aa, synthetic construct
  source 1..447: note = Antibody LK-RB1-5 Heavy Chain
QVQLQQPGAELVKPGASVKLSCKASGYTFTDFWIHWVKQRPGQGLEWIGMIHPNSGSTNFNEKFRAKATLTVDKSSSTAYMRLSSLTSEDSAVYYCARSGDGYSNPMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 80 - protein, 213 aa, synthetic construct
  source 1..213: note = Antibody LK-RB1-5 Light Chain
QIVLSQSPAILSASPGEKVTLTCRASSSVSYMHWYQQKPGSSPKPWIYATSNLASGVPVRFSGSGSGTSYSLTISRMQTEDAATYYCQQWRRSPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 81 - protein, 449 aa, synthetic construct
  source 1..449: note = Antibody LK-RB1-6 Heavy Chain
QIQLVQSGPELKKPGETVKISCKASGYTFTTYGMSWVKQAPGKDLKWMGWINTFSGVPACADDFQGRFDFSAEPSDSTAHLQINNLKNEDTATYFCARRSYYGSRGWYFDVWGTGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 82 - protein, 214 aa, synthetic construct
  source 1..214: note = Antibody LK-RB1-6 Light Chain
DIVMTQSHKFMSTSVGDRVSITCTASEDVNTAVAWYQQKPGQSPKLLIYWASTRHTGVPDRFTGSGSGTDYTLTISSVQTEDLALYYCQQHYNTPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 83 - protein, 449 aa, synthetic construct; identical to SEQ ID NO: 81
  source 1..449: note = Antibody LK-RB1-7 Heavy Chain

SEQ ID NO: 84 - protein, 214 aa, synthetic construct
  source 1..214: note = Antibody LK-RB1-7 Light Chain
DVQMIQSPSSLSASLGDIVTMTCQASQDTTINLNWFQQKPGKVPKLLIYGASNLEDGVPSRFSGSSFGTDFTLTISSLEDEDMATYFCLQHTYLPRTFGGGTKLEIRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 85 - protein, 447 aa, synthetic construct
  source 1..447: note = Antibody LK-RB1-8 Heavy Chain
QVQLQQSGAELVRPGTSVKVSCKASGYAFSNYLIEWVKQRPGQGLEWIGVINPGSDGSTYNEKFKGKATLTADKSSNTAYMQLSSLTSEDSAVYFCARGDYGRSYGMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 86 - protein, 214 aa, synthetic construct
  source 1..214: note = Antibody LK-RB1-8 Light Chain
DIVMTQSQKFMSTTVGDRVSITCKASQNVGTAVVWYQQKPGQSPKLLIYSASNRYTGVPDRFTGSGSGTDFTLTISNMQSEDLADYFCQQYSSYMYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 87 - protein, 449 aa, synthetic construct
  source 1..449: note = Antibody LK-RB1-9 Heavy Chain
QIQLVQSGPELKKPGETVKISCTASGYTFTTYGMSWVKQAPGKGLKWMGWINTYSGVPTYADDFKGRFAFSLETSASTAYLEINNVKNEDTATYFCARRRRTYHGYLCFDYWGQGTTLTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 88 - protein, 214 aa, synthetic construct
  source 1..214: note = Antibody LK-RB1-9 Light Chain
DIQMTQSPVSLSASVGETVTITCETSENIYGALNWYQRKQGKSPQLLIYGATNLADGMSSRFSGSGSGRQYSLEISSLHPDDVASYYCQNVLNTPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 89 - protein, 445 aa, synthetic construct
  source 1..445: note = Antibody LK-RB1-10 Heavy Chain
QVTLKESGPGILKPSQTLSLTCSFSGFSLSTSGTGVGWIRQPSGKGLEWLAHIWWNDDKYYKPSLKRQLTISKDTSRNQVFLKITSVDTADTATYYCTRTELGRGDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 90 - protein, 214 aa, synthetic construct
  source 1..214: note = Antibody LK-RB1-10 Light Chain
DILLTQSPAILSVSPGERVSFSCRASQSIGTNIHWYQQRMNGSPRLIIKYASELISGIPSRFSGSGSGTDFTLSINSVESEDIADYYCQQSYGWPYTFGGGTKLETKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 91 - protein, 449 aa, synthetic construct
  source 1..449: note = Antibody LK-RB1-11 Heavy Chain
QVTLKESGPGILQPSQTLSLTCSFSGFSLKTSGMSVGWIRQPSGKGLEWLAHIWWNDDKYYNPVLKGRLTISKDTSNNLVFLKIASVVSTDTATYYCVRYGNSHGYWYFDVWGTGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 92 - protein, 218 aa, synthetic construct
  source 1..218: note = Antibody LK-RB1-11 Light Chain
NIVLTQSPASLAVSLGQRATISCRASESVDSYGNNFIHWYQQKPGQPPKVLIYYASNLEAGVPARFSGSGSRTDFTLTIDPVEAEDAATYYCQQNNEDPYTFGGGTKLEIERTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 93 - protein, 447 aa, synthetic construct
  source 1..447: note = Antibody LK-RB1-12 Heavy Chain
QVQLQQSGTELVRPGSSVMISCKASGYAFSTYWMDWVKQRPGQGLEWIGQIYPGDGATNYNGKFKGKATLTADKSSITAYMHLSSLTSEDSAVYFCLRSRDYDGWFFDVWGAGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 94 - protein, 214 aa, synthetic construct
  source 1..214: note = Antibody LK-RB1-12 Light Chain
DIVMTQSHKFMSTSIGDRVSITCKASQDVSSAVAWYQQKPGQSPKLLIYWASTRHTGVPDRFTGSGSGTDYTLTFSSVQAEDLALYYCQQHYSTPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 95 - protein, 445 aa, synthetic construct
  source 1..445: note = Antibody LK-RB1-13 Heavy Chain
QVTLKESGPGIVKPSQTLSLTCSFSGFSLSISGTGIGWIRQPSGKGLEWLAHIWWDDDKYYNPSLKSQVTISKDTSTKQVFLNITSVDTADTATYYCTRTELGRGDFWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 96 - protein, 214 aa, synthetic construct
  source 1..214: note = Antibody LK-RB1-13 Light Chain
DILLTQSPAILSVSPGDRVSFSCRASQSTGTNIHWYQRRTNGSPRLLIKYGSESISGIPSRFSGSGTGTDFTLTINSVESEDIADYYCQQSYSWPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 97 - protein, 445 aa, synthetic construct
  source 1..445: note = Antibody LK-RB1-14 Heavy Chain
QVTLKESGPGILKPSQTLSLTCSFSGFSLSTSGMGVGWIRQPSGKGLEWLAHIWWDDDKYYNPSLKSQLTISKDTSRNQVFLKITSVDTADTATYYCARTELGRGDSWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 98 - protein, 214 aa, synthetic construct
  source 1..214: note = Antibody LK-RB1-14 Light Chain
DILLTQSPAILSVSPGERVSFSCRASQSIGTSIHWYQQRTNGSPRLLIKYASESISGIPSRFSGSGSGTDFTLSINSVESEDIADYYCQQSYSWPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 99 - protein, 445 aa, synthetic construct; identical to SEQ ID NO: 95
  source 1..445: note = Antibody LK-RB1-15 Heavy Chain

SEQ ID NO: 100 - protein, 214 aa, synthetic construct
  source 1..214: note = Antibody LK-RB1-15 Light Chain
DILLTQSPAILSVSPGDRVSFSCRASQSIGTNIHWYQRRTNGSPRLLIKYGSESISGIPSRFSGSGTGTDFTLTINSVESEDIADYYCQQSYSWPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 101 - protein, 445 aa, synthetic construct
  source 1..445: note = Antibody LK-RB1-16 Heavy Chain
QVQLKESGPGLVAPSQSLSITCTVSGFSLISNAISWVRQPPGKGLEWLGVIWGDGNTIYHSALRSRLTISKDNSKSQVFLKLNSLQIDDTATYYCAKANWDGYYFDYWGQGTTLTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 102 - protein, 214 aa, synthetic construct
  source 1..214: note = Antibody LK-RB1-16 Light Chain
DIQMNQSPSSLSASLGDTITITCHASQSVDFWLNWYQQKPGNIPKLLISKASNLHTGVPSRFSGSGSGTTFTLTISSLQPEDIATYYCQQGQSYPLTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 103 - protein, 446 aa, synthetic construct
  source 1..446: note = Antibody LK-RB1-17 Heavy Chain
QVQLQQPGAELVKPGASVKLSCEASGYTFTNYWIHWVKQRPGRGLEWIGRIAPNSGGTKYNEKFKTRATLTVDKASTTAYMQLSSLTSEDSAVYYCARLDGSPYYFDYWGQGTTLTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 104 - protein, 214 aa, synthetic construct
  source 1..214: note = Antibody LK-RB1-17 Light Chain
DIVMTQSQKFMSTSIGDRVSVTCKASQNVDTNVAWYQQKPGQSPKALIYSASYRYSGIPDRFTGSGSGTDFTLTISNVQSEDLAEYFCQQYNSYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 105 - protein, 445 aa, synthetic construct
  source 1..445: note = Antibody LK-RB1-18 Heavy Chain
QVQLKESGPGLVAPSQSLSITCTVSGFSLTNSGVTWIRQPPGKGLEWLGVIWGDGSTNYHSALISRLSISKDNSKSQVFLKLNSLQTDDTATYYCAKPHWDYYNMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 106 - protein, 214 aa, synthetic construct
  source 1..214: note = Antibody LK-RB1-18 Light Chain
DIQMTQTPSSLSASLGDRVTISCRASQDISNYLNWYQQKPDGTVKLLIYFTSRLHSGVPSRFSGSGSGTDYSLTISNLEQEDFATYFCQQGITLPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 107 - protein, 445 aa, synthetic construct
  source 1..445: note = Antibody LK-RB1-19 Heavy Chain
QVQLKESGPGLVAPSQNLSITCTVSGFSLTSYGVSWVRQPPGKGLEWLGIIWGDGNTDYHSALISRLSIRKDNSKSQVFLKLNSLKSDDTATYYCAKPHWEYYTMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 108 - protein, 214 aa, synthetic construct
  source 1..214: note = Antibody LK-RB1-19 Light Chain
DIQMTQTTSSLSASLGDRVTISCRASQDISNFLNWYQQKPDGTVKLLIYSTSRLHSGVPSRFSGSGSGTDYSLTIANLEQEDVATYFCQQGNTLPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 109 - protein, 445 aa, synthetic construct
  source 1..445: note = Antibody LK-RB1-20 Heavy Chain
QVQLKESGPGLVAPSQSLSITCTVSGFSLNSNGVSWVRQPPGKGLEWLGLIWGDGSTNYHSALISRLSIRKDNSKSQVFLKLNSLQNDDTATYYCAKPNWDGYHFDYWGQGTTLIVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 110 - protein, 214 aa, synthetic construct
  source 1..214: note = Antibody LK-RB1-20 Light Chain
DIQMNQSPSSLSASLGDTITITCHASQNIDFWLSWYQQKPGNIPKLLIYKASNLHTGVPSRFSGSGSGTGFTLTISSLQPEDIATYYCQQGQSYPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 111 - protein, 446 aa, synthetic construct
  source 1..446: note = Antibody LK-RB1-21 Heavy Chain
QVQLQQPGAELVKPGASVKMSCKASGYIFTDYWTTWVKQRPGQGLEWIGDIYPVSGSTNYNEKFKNKATLTVDTSSSTAYMQLSSLTSEDSAVYYCARALGRRYYFDYWGQGTTLTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 112 - protein, 214 aa, synthetic construct
  source 1..214: note = Antibody LK-RB1-21 Light Chain
DIVMTQSHKFMSTSIGDRVSITCKASQDVSTAVAWCQQKPGQSPKSLIYSASYRYTGIPDRFTGSGSGTDFTFTISSVQAEDLAVYYCQQHYTNPVTFGTGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 113 - protein, 445 aa, synthetic construct
  source 1..445: note = Antibody LK-RB1-22 Heavy Chain
QVQLKESGPGLVAPSQSLSITCTVSGFSLTSYGVSWVRQPPGKGLEWLGIIWGDGNTNYHSALTSRLSISKDNSKSQVFLKLNSLRTDDTATYYCAKPHWDHYSMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 114 - protein, 214 aa, synthetic construct
  source 1..214: note = Antibody LK-RB1-22 Light Chain
DIQMTQTTSSLSASLGDRVTISCRASQDISNFLNWYQQKPDGTVKLLIYYTSRLHSGAPSRFSGSGSGTDYSLTISNLDQEDIATYFCQQGNTLPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 115 - protein, 444 aa, synthetic construct
  source 1..444: note = Antibody LK-RB1-23 Heavy Chain
QVQLQQTGAELVRPGSSVKLSCKASGYTFTNYWMDWVKQRPGQGLEWIGNIHPFGNETHYNQKFKDKATLTVDKSSSTAYMQLSSLTSEDSAVYYCARGLLLYFDVWGTGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 116 - protein, 214 aa, synthetic construct
  source 1..214: note = Antibody LK-RB1-23 Light Chain
DIQMTQSPASQSVSVGETVTITCRASENIYSNLAWYQQKQGKSPQLLVYGATNLADGVSLRFSGSGSGTQYSLKINSLQSEDFGSYYCQHFWGIPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 117 - protein, 441 aa, synthetic construct
  source 1..441: note = Antibody LK-RB1-24 Heavy Chain
QVQLQQPGAELVNPGTSVNLSCKASGYTFTGYWMHWVKQRPGQGLEWIGMIHPNNGGTIYNERFNGKATLTVDRSSTTAYIQLSSLTSEDSAVYYCAYWDLDYWGQGTTLTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 118 - protein, 219 aa, synthetic construct
  source 1..219: note = Antibody LK-RB1-24 Light Chain
DVMMTQTPLTLSVTIGQPASISCKSSQSLLNSDGKTYLNWLLQRPGQSPKRLIYLVSKLGSGVPDRFTGSGSGTDFTLKISRVEAEDLGVYYCWQGTHFPWTFGGGAKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 119 - protein, 448 aa, synthetic construct
  source 1..448: note = Antibody LK-RB1-25 Heavy Chain
QIQLVQSGPELKKPGETVKISCKASGYTFTSYGMNWMKQAPGEGLKWMGWINTYSGVPTYADDFKGRFAFSLETSATTVYLQINNLKNEDKATYFCARRGRDSVVGHFDVWGTGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 120 - protein, 214 aa, synthetic construct
  source 1..214: note = Antibody LK-RB1-25 Light Chain
DIVMTQSHKFMSTSVGDRVSITCKASQDVNTAVAWYQQKPGQSPKLLIYWASTRHTGVPDRFTGSGSGTDFTLTISSVQAEDLALYYCQQHYTIPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 121 - protein, 445 aa, synthetic construct
  source 1..445: note = Antibody LK-RB1-26 Heavy Chain
QVQLKESGPGLVAPSQSLSITCTVSGFSLTSYGVSWVRQPPGKGLEWLGIIWGDGSTNYHSALISRLSISKDNSKSQVFLKLNSLQTDDTATYYCAKPNWDYYAMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 122 - protein, 214 aa, synthetic construct
  source 1..214: note = Antibody LK-RB1-26 Light Chain
DIQMTQTTSSLSASLGDRVTISCRASQDISNFLNWYQQKPDGTFKLLISYTSTLHSGVPSRFSGSGSGTDYSLTISNLEQEDVATYFCQQGNTLPLTFGAGTKLALKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 123 - protein, 444 aa, synthetic construct
  source 1..444: note = Antibody LK-RB1-27 Heavy Chain
QVQLQQSGPELVKPGASVRMSCKASGYTFTSHYIHWMKQRPGQGLEWIGWIYPGDGHTKYHEKFKDRATLTADTSSITAYMRLSSLTSEDSAIYYCVYYSDSWFPYWGQGTLVTVSAASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 124 - protein, 214 aa, synthetic construct
  source 1..214: note = Antibody LK-RB1-27 Light Chain
DIQMTQSPASLSVSVGETVTITCRASENIYSNLAWYQQKQGKSPHLLVYAATNLADGVPSRFSGSGSGTQYSLKINSLQSEDFGNYYCQHFWGSPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 125 - protein, 444 aa, synthetic construct
  source 1..444: note = Antibody LK-RB1-28 Heavy Chain
QVQLQQSGPELVKPGTSVKMSCKASGYTFTSHYIHWMKQRPGQGLDWIGWIYPGDGHTKYHEKFKGKATLTADTSSITAYMQLSLLTSEDSAVYYCVYYSDSWFPYWGQGTLVTVSAASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 126 - protein, 214 aa, synthetic construct
  source 1..214: note = Antibody LK-RB1-28 Light Chain
DIQMTQSPASLSVSVGETVTITCRASENIYSNLAWYQQKQGKSPQLLVYAATNLADGVPSRFSGSGSGTQYSLKINSLQSEDFGNYFCQHFWGSPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 127 - protein, 445 aa, synthetic construct
  source 1..445: note = Antibody LK-RB1-29 Heavy Chain
QVQLKESGPGLVAPSQNLAITCTVSGFSLTNSGVSWVRQSPGKGLEWLGIIWGDGNTNYHSALISRLSISKDNSKSQVFLKLNSLQTDDTGTYYCAKPNWDYYALDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 128 - protein, 214 aa, synthetic construct
  source 1..214: note = Antibody LK-RB1-29 Light Chain
DIQMTQTASSLFASLGDRVTISCRASQDISNYLNWYQQKPDGSIKLLISYTSRLHSGVPSRFSGSGSGTDYSLSISNLEQEDIATYFCQQGISLPCTFGGGTKLEVNRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 129 - protein, 445 aa, synthetic construct
  source 1..445: note = Antibody LK-RB1-30 Heavy Chain
QVQLKESGPGLVAPSQSLSITCTVSGFSLTSYGVSWIRQPPGKGLEWLGVIWGDGNTNYHSALISRLSISKDNSKSQVFLKLNSLQTDDTATYYCAKPSWDYYAMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 130 - protein, 214 aa, synthetic construct
  source 1..214: note = Antibody LK-RB1-30 Light Chain
VIQMTQTTSSLSASLGDRVTISCRASQDISNYVNWYQQRPDGTVKLLISYTSRLHSGVPSRFSGSGSGTDYSLTITNLEQEDIATYFCQQGITLPCTFGGGTKLEINRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 131 - protein, 445 aa, synthetic construct
  source 1..445: note = Antibody LK-RB1-31 Heavy Chain
QVQLKESGPGLVAPSQSLSITCTVSGFSLTSYGVSWVRQPPGKGLEWLGIIWGDGSTNYHSALKSRLSISKDNSKSQVFLKLNSLQTEDTATYYCAKPNWDYYAMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 132 - protein, 214 aa, synthetic construct
  source 1..214: note = Antibody LK-RB1-31 Light Chain
DIQMTQTTSSLSASLGDRVTISCRASQDISNYLNWYLQKPDGTVKLLIYYTSRLHSGVPSRFSGSGSGTDYSLTISNLEQEDIATYFCQQGITLPCTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 133 - protein, 444 aa, synthetic construct
  source 1..444: note = Antibody LK-RB1-32 Heavy Chain
QVQLQQSGAELAKPGASVKLSCTASGYTFTTYLMHWVKQRPAQGLEWIGYINPSNDFTKYNQKFKDKATLTADTSSSTAYMQLNSLTFEDSAVYYCARNSDYDPSYWGQGTTLTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 134 - protein, 213 aa, synthetic construct
  source 1..213: note = Antibody LK-RB1-32 Light Chain
DIQMTQSPASLSVSVGETVTITCRASENIYSSLAWYQQKQGKSPQLLVYAATNLADGVPSRFSGSGSGTQYSLKINSLQSEDFGNYYCQHFWGTWAFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 135 - protein, 448 aa, synthetic construct
  source 1..448: note = Antibody LK-RB1-33 Heavy Chain
QIQLVQSGPELKKPGDTVKISCKASGYTFTSYGMNWVKQAPGEGLKWMGWINTYSGVPTYGDDFKGRFAFSLETSASTAYLQINNLENEDTATYFCARRGRDSVVGHFDVWGTGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 136 - protein, 214 aa, synthetic construct
  source 1..214: note = Antibody LK-RB1-33 Light Chain
DIVMTQSHKFMSTSVGDRVSITCKASQDVNTAVAWYQQKPGQSPKLLIYWASTRHTGVPDRFTGSGSGTDFTLTISSVQAEDLALYYCQQHYTIPFTFGSGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 137 - protein, 450 aa, synthetic construct
  source 1..450: note = Antibody LK-RB1-34 Heavy Chain
QVQLQQSGAELVKPGASVKLSCKASGYTFTEYTIHWVKQRSGQGLEWIGWFYPGSDSIKYNEKFKDKATLTADKSSSTVYMELSRLTSEDSAVYFCSRHEGPYFTSQYYFDYWGQGTTLTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 138 - protein, 214 aa, synthetic construct
  source 1..214: note = Antibody LK-RB1-34 Light Chain
DILLTQSPAILSVSPGERVSFSCRASRYIGTSIHWYQQRTNGSPRLLIKYASESISGIPSRFSGSGSGTDFTLSINSVESEDIADYYCQQSNTWPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 139 - protein, 444 aa, synthetic construct
  source 1..444: note = Antibody LK-RB1-35 Heavy Chain
DVQLQESGPGLEKPSQSLSLTCSVTGYSITRGYQWNWIRQFPGNKLEWMGYISYDGSNNYNPSLKNRISITRDASKNQFFLKLNSVTTEDTATYYCARRSYNSIDYWGQGTTLTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 140 - protein, 214 aa, synthetic construct
  source 1..214: note = Antibody LK-RB1-35 Light Chain
DIVLTQSPATLSVTPGDSVSLSCRASQSISSNLHWYQQKSHESPRLLIKYASQSISGIPSRFSGSGSGTDFTLSINSVETEDFGMYFCQQSNSWPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 141 - protein, 444 aa, synthetic construct
  source 1..444: note = Antibody LK-RB1-36 Heavy Chain
DVQLQESGPGLEKPSQSLSLTCSVTGYSITRGYQWNWIRQFPGNKLEWMGYISYDGSNNYNPSLKNRISIIRDTSKNQFFLKLNSVTTEDTATYYCARRSYNSIDYWGQGTTLTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 142 - protein, 214 aa, synthetic construct
  source 1..214: note = Antibody LK-RB1-36 Light Chain
DIVLTQSPATLSVTPGDSVSLSCRASQSISNNLHWYQQKSHESPRLLIKYASQSISGIPSRFSGSGSGTDFTLSINSVETEDFGMYFCQQSNSWPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 143 - protein, 448 aa, synthetic construct
  source 1..448: note = Antibody LK-RB1-37 Heavy Chain
QVQLQQPGAELVKPGASVKLSCKASGYTFTSYWMHWVKQRPGRGPEWIGRIDPNSGYARYNEKFKNKATLTADKPSSTADMHLSSLTSEDSAVYYCTRSFITTAEWFFDVWGTGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 144 - protein, 214 aa, synthetic construct
  source 1..214: note = Antibody LK-RB1-37 Light Chain
DILLTQSPAILSVSPGERVSFSCRASQSIGTSIHWYQQRRNGSPRLLIKYTSESISGIPSRFSGSGSGTDFTLSINSVESEDIADYYCQQSNSWPHTFGGGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 145 - protein, 444 aa, synthetic construct
  source 1..444: note = Antibody LK-RB1-38 Heavy Chain
DVQLQESGPGLVQPSQSLSLTCSVTGYSITSGYQWNWIRQLPGNKLEWMAYISFDGSNNYNPSLKNRISITRDTSKNQFFLKLKSVTTEDTATYYCARRDYDGVAYWGQGTLVTVSAASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 146 - protein, 214 aa, synthetic construct
  source 1..214: note = Antibody LK-RB1-38 Light Chain
EIVLTQSPATLSVTPGDRVSLSCRASQNISNYLHWYQQKSHGSPRLIIKYASQSISGIPSRFSGSGSGTDFTLSINSVETEDFGMYFCQQSNSWPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 147 - protein, 443 aa, synthetic construct
  source 1..443: note = Antibody LK-RB1-39 Heavy Chain
EVQLQQSGAELVRPGASVKLSCTASGFNIKDDYVHWVNQRPEQGLEWIGWIDPENGDTGYASKFQDKATITADTSSNTAYLHLSSLTSEDTAVYYCTTTRWTGSYWGQGTTLTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 148 - protein, 215 aa, synthetic construct
  source 1..215: note = Antibody LK-RB1-39 Light Chain
QIVLTQSPAIMSASPGEKVTITCSASSSVNYMHWFQQKPGSSPKLWIYSTANLASGVPARFSGSGSGTSYSLTISRMEAEDAATYYCQQRSSYPPTWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 149 - protein, 444 aa, synthetic construct; identical to SEQ ID NO: 145
  source 1..444: note = Antibody LK-RB1-40 Heavy Chain

SEQ ID NO: 150 - protein, 219 aa, synthetic construct
  source 1..219: note = Antibody LK-RB1-40 Light Chain
DVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 151 - protein, 444 aa, synthetic construct; identical to SEQ ID NO: 145
  source 1..444: note = Antibody LK-RB1-41 Heavy Chain

SEQ ID NO: 152 - protein, 214 aa, synthetic construct; identical to SEQ ID NO: 146
  source 1..214: note = Antibody LK-RB1-41 Light Chain

SEQ ID NO: 153 - protein, 448 aa, synthetic construct
  source 1..448: note = Antibody LK-RB1-42 Heavy Chain
QVQLQQPGAELVKPGTSVKLSCKASGYSFISYWVYWVKQRPGQGLEWIGMIHPNSGSTNFNEKFRYKATLTVDKSSSTAYIQLSSLTSEDSAVYYCGRSDDGYPYHAMDSWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 154 - protein, 214 aa, synthetic construct
  source 1..214: note = Antibody LK-RB1-42 Light Chain
DIVMTQSQKFMSTSVGDRVSVTCKASQNVGTNVAWYQQKPGQSPKALIYSASYRFSGVPDRFTGSGSGTDFTLTIGNVQSEDLAEYFCHQYKNYPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 155 - protein, 445 aa, synthetic construct
  source 1..445: note = Antibody LK-RB1-43 Heavy Chain
EFQLQQSGPELMKPGASVKISCKASGYSFTDYNMNWVKQSNGKSLEWIGIIDPNYGTTTYNQKFKGRATLTVDQSSNTAYMQLNSLTSEDSAVYYCAREGRYSWFAYWGQGTLVTVSAASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 156 - protein, 214 aa, synthetic construct
  source 1..214: note = Antibody LK-RB1-43 Light Chain
DIQMTQSPGALSVSVGETVTITCRASENINSNLAWYQQKQGKSPQLLVYAATNLADGVPSRFSGSGSGTQYSLKINSLQSEDFGSYHCQHFWGTPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 157 - protein, 448 aa, synthetic construct
  source 1..448: note = Antibody LK-RB1-44 Heavy Chain
QVQLQQSGTELAKPGASVKLSCKASGYTFTNYWMHWIKQRPGQGLEWIGYVHPTSDYTKYNQKFKDKATLTADKSSSTAYMQLNSLTYEDSAVYYCTRSSLYDTNYVFDSWGQGTTLTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 158 - protein, 214 aa, synthetic construct
  source 1..214: note = Antibody LK-RB1-44 Light Chain
DILLTQSPAILSVSPGERVSFSCRASQSIGRSIHWFQQRTNGSPSLLIKYASESISGIPSRFSGGGSGTDFTLSISNVESEDIADYYCQQSNNWPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 159 - protein, 448 aa, synthetic construct
  source 1..448: note = Antibody LK-RB1-45 Heavy Chain
QVQLQQSGTELARPGASVKLSCKASGYTFTNFWMHWIKQRPGQGLEWIGYVNPTSDYTKYNQKFKDKATLTADKSSSTAYMQLNSLTYEDSAVYYCTRSSLYDTNYVFDSWGQGTTLTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 160 - protein, 214 aa, synthetic construct
  source 1..214: note = Antibody LK-RB1-45 Light Chain
DILLTQSPAILSVSPGERVSFSCRASQSIGRSIHWYQQRTNGSPRLLIKYASESISGIPSRFSGGGSGTDFTLSISNVESEDIADYYCQQSNNWPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 161 - protein, 447 aa, synthetic construct
  source 1..447: note = Antibody LK-RB1-46 Heavy Chain
EVQLQQSGPELVKPGASVKISCKTSGYTFTDYYMNWVKQSHGKSLEWIGDIYPINGDISYNQKFKGKATLTVDKSSSTVYMEIRSLTYEDSAVYYCARLAQTTSGGFDYWGQGTTLTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 162 - protein, 219 aa, synthetic construct
  source 1..219: note = Antibody LK-RB1-46 Light Chain
DIVLTQSPTSLAVSLGQRAIISCKASQSVSFAGTSLMHWYQQKPGQQPKLLIYRASNLKAGVPTRFSGSGSRTDFTLNIHPVEEDDAATYYCQQSREYPMYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 163 - protein, 449 aa, synthetic construct
  source 1..449: note = Antibody LK-RB1-47 Heavy Chain
QIQLVQSGPELKKPGETVKISCKASGYTFTTYGMSWVKQAPGKGLKWMGWINTYSGVPTYTDDFKGRFAFSVETSASTAYLQINNLKNEDTATYFCARRERDFYGNIFFPYWGQGSLVTVSAASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 164 - protein, 214 aa, synthetic construct
  source 1..214: note = Antibody LK-RB1-47 Light Chain
DIQMTQTTSSLSASLGDRVTISCRASQDISNYLNWYQQKPDGTVKLLIYYTSRIHSGVPSRFSGSGSGTDFSLTISNLDQEDIATYFCQQGHTLPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 165 - protein, 446 aa, synthetic construct
  source 1..446: note = Antibody LK-RB1-48 Heavy Chain
EVKLVESGGGLVQPGGSLKLSCAASGFTFSDYYMYWVRQTPEKRLEWVAYISNGGGISFYPDTVKGRFTISRDNAKNTLYLQMSRLKSEDTAIYYCASATKEVGAMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 166 - protein, 213 aa, synthetic construct
  source 1..213: note = Antibody LK-RB1-48 Light Chain
DIVMTQSQKFLSTTVGDRVSITCKASQNVGTAVAWYQQKPGQSPKILIYSASNRYTGVPDRFTGSGSGTDFTLTISNMQSEDLADYFCQQYSSYRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 167 - protein, 448 aa, synthetic construct
  source 1..448: note = Antibody LK-RB1-49 Heavy Chain
QIQLVQSGPELKKPGETVKISCKASGYTFTTYGMSWVKQAPGKGLKWMGWINTYSGVPTYADDFKGRFAFSLETSGSTAYLQISNLKNEDTATYFCSRLFRSWDSYAMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 168 - protein, 214 aa, synthetic construct
  source 1..214: note = Antibody LK-RB1-49 Light Chain
DIVMTQSQKFMSATVGDRVSITCKASQNVGTAVAWYQQKPGQSPKLLIYSPSNRYTGVPDRFTGSGSGTHFTLTINNMQSEDLADYFCQQYSTYPLTFGAGTKLELNRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 169 - protein, 448 aa, synthetic construct
  source 1..448: note = Antibody LK-RB1-50 Heavy Chain
QVQLKQSGPGLVQPSQSLSITCTVSGFSLTSYGVHWVRQSPGKGLEWLGVIWRGGNTDYNAPFVSRLSITKDNSQSQVFFKMNSLEADDTAIYYCAKNSLDVYSSWYFDVWGTGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 170 - protein, 213 aa, synthetic construct
  source 1..213: note = Antibody LK-RB1-50 Light Chain
QIVLSQSPAILSAFPGEKVTMTCRASSSVSFMHWYQQKPGSSPKPWIYATSNLASGVPARFSGSGSGTSYSLTISRVEAEDAATYYCQQWSSNPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 171 - protein, 447 aa, synthetic construct
  source 1..447: note = Antibody LK-RB1-51 Heavy Chain
QVQIQQSGAELVKPGASVKLSCKASGYTFTEFPIHWIKQRSGQGLEWIGWFYPGSDNMNYNEKFKDKATLTADKSSSTVYMELSRLTSEDSAVYFCARHENAYFYAMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 172 - protein, 213 aa, synthetic construct
  source 1..213: note = Antibody LK-RB1-51 Light Chain
ENVLTQSPAIMSASLGEKVTMSCRASSSVNYMYWYQQKSDASPKIWIYYTSNLAPGVPARFSGSGSGNSYSLTISSMEGEDAATYYCQQFTSSPFTFGTGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 173 - protein, 446 aa, synthetic construct
  source 1..446: note = Antibody LK-RB1-52 Heavy Chain
EVKLVESGGGLVQPGGSLKLSCAASGFIFSDYYMYWVRQTPEKRLEWVAYISNGGDITYYPDTVKGRFTISRDIARNTLFLQMGRLKSEDTAMYYCASATTEVGAMDHWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 174 - protein, 213 aa, synthetic construct
  source 1..213: note = Antibody LK-RB1-52 Light Chain
DIVMTQSQKFMSTTVGDRVSITCKASQNVGTAVAWYQQKPGQSPKLLIYAASNRYTGVPDRFTGSGSGTDFTLTISNMQSEDLADYFCQQYRSYRTFGGGTNLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 175 - protein, 450 aa, synthetic construct
  source 1..450: note = Antibody LK-RB1-53 Heavy Chain
QIQLVQSGPELKKPGETVKISCKASGYTFTTYGMSWVNQAPGKGLKWMGWINTNYGVPTYADDFKGRFAFSLETSASTAYLQINNLKNEDTATYFCARGTRRRGISYFAMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 176 - protein, 214 aa, synthetic construct
  source 1..214: note = Antibody LK-RB1-53 Light Chain
DIVMTQSQKFMSTTVGDRVSITCKASQNVGTAVAWYQQKPGQSPKLLIYSASNRYTGVPDRFTGSDSGTDFTLTINNIQSEDLADYFCQQYSSYPLTFATGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 177 - protein, 447 aa, synthetic construct
  source 1..447: note = Antibody LK-RB1-54 Heavy Chain
DVQLQESGPGLVKPSQSLSLTCSVTGYSITSGFYWNWIRQFPGNKLEWMGHISYDGTNNYNPSLKNRISITRDTSKNLFFLKLNSVTTEDTATFHCARGGYGGSYGFDYWGQGTTLTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 178 - protein, 213 aa, synthetic construct
  source 1..213: note = Antibody LK-RB1-54 Light Chain
ENVLTQSPAIMSASLGEKVTMSCRASSSVNYMYWYQQKSDASPKLWIYYTSNLAPGVPVRFSGSGSGNSYSLTISSMEGEDAATYYCQQFTSSPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 179 - protein, 446 aa, synthetic construct
  source 1..446: note = Antibody LK-RB1-55 Heavy Chain
EVKLVESGGGLVQPGGSLKLSCAASGFTFSDYYMYWVRQTPEKRLEWVAYISNGGDITFYPDTVKGRFTISRDNARSTLYLQMSRLKSEDTAIYYCASATTEVGAMDHWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 180 - protein, 213 aa, synthetic construct
  source 1..213: note = Antibody LK-RB1-55 Light Chain
DIVMTQSQKFMSTTVGDRVSITCKASQNVGTAVAWYQQKPGKSPKLLIYAASNRYTGVPDRFTGSGSGTDFTLTISNMQSEDLADYFCQQYSSYRTFGGGTNLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 181 - protein, 448 aa, synthetic construct
  source 1..448: note = Antibody LK-RB1-56 Heavy Chain
QVQLKQSGPGLVQPSQSLSITCSVSGFSLTNYGVHWVRQSPGKGLEWLGVIWRGGSTDNNAAFMSRLSITKDNSKSQVFFKMNSLQADDTAIYYCAKNSDFYGSSWYFDVWGTGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 182 - protein, 213 aa, synthetic construct
  source 1..213: note = Antibody LK-RB1-56 Light Chain
QIVLSQSPAILSASPGEKVTMTCRASSSVSFMHWYQQKPGSSPKPWIYATSNLASGVPARFSGSGSGTSYSLTISRVEAEDAATYYCQQWSRNPPTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 183 - protein, 446 aa, synthetic construct
  source 1..446: note = Antibody LK-RB1-57 Heavy Chain
EVKLVESGGGLVQPGGSLKLSCAASGFTFSDYYMYWVRQTPEKRLEWVAYISNGGDITFYPDTVKGRFTISRDNARSTLYLQTSRLKSEDTAIYYCASATTEVGAMDHWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 184 - protein, 213 aa, synthetic construct
  source 1..213: note = Antibody LK-RB1-57 Light Chain
DIVMTQSQKFMSTTVGDRVSITCKASQNVGTAVAWYQQKPGKSPKLLIYAASNRYTGVPDRFTGSGSGTDFTLTISNVQSEDLADYFCQQYSSYRTFGGGTSLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 185 - protein, 448 aa, synthetic construct
  source 1..448: note = Antibody LK-RB1-58 Heavy Chain
QVQLKQSGPGLVQPSQSLSITCTVSGFSLTTYGVHWVRQSPGKGLEWLGVIWRGGSTDYNAAFMSRLSITKDNSKSQVFFRMNSLQADDTARYYCAKNSLYYGSSWYFDVWGTGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 186 - protein, 213 aa, synthetic construct
  source 1..213: note = Antibody LK-RB1-58 Light Chain
QIVLSQSPAILSASPGEKVTMTCRASSSVSFMHWYQQKPGSSPKPWIYATSNLASGVPARFSGSGSGTSYSLTISRVEAEDAATYYCQQWSSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 187 - protein, 450 aa, synthetic construct
  source 1..450: note = Antibody LK-RB1-59 Heavy Chain
QIQLVQSGPELKKPGETVKISCKASGYTFTTYGMSWVKQAPGKGLKWMGWINTYSGVPTYADDFKGRFAFSLETSASTAYLQINNLKNEDTATYFCARRRIYYGNKGYAMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 188 - protein, 214 aa, synthetic construct
  source 1..214: note = Antibody LK-RB1-59 Light Chain
DIQMTQSPASLSVSVGETVTITCRASENIYSNLVWYQQKQGKSPQLLVYTATNLADGVPSRFSGSGSGTQFSLKINSLQSEDFGSYYCQHFWGTPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 189 - protein, 447 aa, synthetic construct
  source 1..447: note = Antibody LK-RB2-1 Heavy Chain
QIQLVQSGPELKKPGETVKISCKASGYTFTTYGMSWVKQAPGKGLKWMGWINTYSGVSTYADDFKGRFAFSLETSASTAYLQINNLKNEDTATYFCARRYDYDGYAMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 190 - protein, 220 aa, synthetic construct
  source 1..220: note = Antibody LK-RB2-1 Light Chain
DIVMSQSPSSLAVSVGEKVTMSCKSSQSLLFSSNQKNYLAWYQQKPGQSPKLLIYWASTRESGVPDRFTGRGSGTDFTLTISSVKTEDLAVYYCQQYDTYPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 191 - protein, 451 aa, synthetic construct
  source 1..451: note = Antibody LK-RB2-2 Heavy Chain
DVKLEESGEGLVKPGGSLKLSCAASGFTFSSYAMSWVRQTPEKRLEWVAYISSGGDYFHYADTVKGRFTISRDNARNTLYLQMSSLKSEDTAMYYCTREGAHYDSSYLAWFAHWGQGTLVTVSAASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 192 - protein, 214 aa, synthetic construct
  source 1..214: note = Antibody LK-RB2-2 Light Chain
DIQMTQSPASLSVSVGETVTITCRASENIYSNLAWYQQRQGKSPQLLVYAATNLADGVPSRFSGSGSGTQYSLKINSLQSEDFGSYYCQQFWDNPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 193 - protein, 441 aa, synthetic construct
  source 1..441: note = Antibody LK-RB2-3 Heavy Chain
QLQLQQSDAELVKPGASVKISCKVSGYSFTDHTIHWMKQRPEQGLEWIGYIYPRDGSTKENEKFTGKATLTADKSSSTAYMQLTSLTSEDSAVYFCTGSGFDYWGQGTSLTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 194 - protein, 214 aa, synthetic construct
  source 1..214: note = Antibody LK-RB2-3 Light Chain
DIKMTQSPSSMYASLGERVTITCKASQDIKSYLSWFQQKPGKSPKTLIYRANRLVDGVPSRFSGSGSGQDYSLTINSLEYEDMEIYYCLQYDEFPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 195 - protein, 450 aa, synthetic construct
  source 1..450: note = Antibody LK-RB2-4 Heavy Chain
EVQLVESGGDLVKPGGSLKLSCAASGFTFSNYGMSWVRQTPDKRLEWVATISSGGSYTFYPDSVKGRFTISRDNAKNTLYLQMSSLKSEDTALYYCARDYYYGKTIEGYFDYWGQGTTLTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 196 - protein, 214 aa, synthetic construct
  source 1..214: note = Antibody LK-RB2-4 Light Chain
DIQMTQTTSSLSASLGDRVTISCRANQDISNYLNWYQQKPDGTVKLLIYYTSRLHSGVPSRFSGSGSGTDYSLTISDLEQEDIATYFCQQGNTLPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 197 - protein, 448 aa, synthetic construct
  source 1..448: note = Antibody LK-RB2-5 Heavy Chain
QIQLVQSGPELKKPGETVKISCKASGYTFTTYGMSWVKQAPGKGLKWMGWINTYSGVPTYADDFKGRFAFSLETSVSTAYLQINNLKNEDTATYFCARRDGYDGGGNFDYWGQGTTLTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 198 - protein, 214 aa, synthetic construct
  source 1..214: note = Antibody LK-RB2-5 Light Chain
DIVMTQSPATLSVTPGDRVSLSCRASQSISDYLHWYQQKSHESPRLLIKYASQSISGIPSRFSGSGSGSDFTLSINSVEPEDVGVYYCQNGHGFPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 199 - protein, 447 aa, synthetic construct
  source 1..447: note = Antibody LK-RB2-6 Heavy Chain
QIQLVQSGPELKKPGETVKISCKASGYTFTTYGMSWVKQAPGKGLEWMGWINIYSGVPTYADDFKGRFAFSLETSASTAYLQINNLKNEDTATYFCARRGDYDSFYFDYWGQGTTLTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 200 - protein, 220 aa, synthetic construct
  source 1..220: note = Antibody LK-RB2-6 Light Chain
DIVMSQSPSSLAVSVGEKVTVSCKSSQSLLYSYNQKNYLAWYQQKPGQSPKLLIYWSSTRESGVPDRFTGSGSGTDFTLTISSVKAEDLAVYYCQQCYNYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 201 - protein, 451 aa, synthetic construct
  source 1..451: note = Antibody LK-RB2-7 Heavy Chain
DVKLVESGEGLVKPGGSLKLSCAASGFTFSSYAMSWVRQTPEMRLEWVAYISSGGDHFYYADTVKGRFTISRDNARSTLYLQMSSLKSEDTAMYYCTREGASYDSNFLAWFAYWGQGTLVTVSAASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 202 - protein, 214 aa, synthetic construct
  source 1..214: note = Antibody LK-RB2-7 Light Chain
DIQMTQSPASLSVSVGETVTITCRASENIYSNLAWYQQKEGKSPQLLVYGATNLADGVPSRFSGSGSGTQYSLKINSLQSEDFGSYYCQNFWDIPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 203 - protein, 444 aa, synthetic construct
  source 1..444: note = Antibody LK-RB2-8 Heavy Chain
QIQLVQSGPELKKPGETVKISCKASGYSFASYGMGWVKQAPGKGLKWLGWINTHSGVPTYADDFKGRFAFSLETSASTAYLQINNLKNEDTATYFCAHYGRPWFPYWGQGTLVTVSAASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 204 - protein, 214 aa, synthetic construct
  source 1..214: note = Antibody LK-RB2-8 Light Chain
DIQMTQSPASLSASVGETVTITCRTSENIYSYLAWYQQKQGKSPQLLVYNAKSLAEGVPSRFSGSGSGTQFSLRINSLQPEDFGTYYCQQHYETPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 205 - protein, 448 aa, synthetic construct
  source 1..448: note = Antibody LK-RB2-9 Heavy Chain
DVQLQESGPGLVKPSQSLSLTCSVTGYSITSGYYWNWIRQFPGNKLEWMGYIGYDGNNNYNPSLKNRISITRDTSKNQFFLKLNSLTTEDTATYYCAREGSTMVTSWFAYWGQGTLVTVSAASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 206 - protein, 214 aa, synthetic construct
  source 1..214: note = Antibody LK-RB2-9 Light Chain
DIQMTQSPASLSVSVGETVTITCRASENIYSNLAWYQQKQGKSPQLLVYGATNLADGVPSRFSGRGSDTQYSLKINSLQSEDFGSYYCQHFWDTPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 207 - protein, 448 aa, synthetic construct
  source 1..448: note = Antibody LK-RB2-10 Heavy Chain
EVMLVESGGDLVKPGGSLKLSCAASGFTFSANTVSWLRRTPERRLEWVATISGGSDNTYCPDSVKGRFTISRDNARDTLYLQMSSLTSEDTALYFCARHYYDGPNWYFDVWGPGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 208 - protein, 214 aa, synthetic construct
  source 1..214: note = Antibody LK-RB2-10 Light Chain
DIVMTQSQKFMSSTGGDRVTITCKASQNVDTAVAWYQQKPGQSPKLLIYSASNRYIGVPDRFKGSGSGTDFSLTITNIQSEDLAEYFCQQYSSYPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 209 - protein, 445 aa, synthetic construct
  source 1..445: note = Antibody LK-RB2-11 Heavy Chain
QIQLVQSGPELKKPGETVKISCKASGYSFITYGMSWVKQAPGKGLKWMGWINTYSGEPTYGDDFMGRFAFSLETSASTAYLQINNLKNEDTASYFCARYHYDDGMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 210 - protein, 214 aa, synthetic construct
  source 1..214: note = Antibody LK-RB2-11 Light Chain
DIKMTQSPSSMYASLGERVTITCKASQDINNYLSWFQQKPGKSPKTLIFRANRLVDGVPSRFSGSGSGQDYSLTISSLEDEDMGIYYCLQYDEFPWMFGGGTKLEIERTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 211 - protein, 447 aa, synthetic construct
  source 1..447: note = Antibody LK-RB2-12 Heavy Chain
QIQLVQSGPELKKPGETVKISCKASGYTFTTYGMSWVKQTPGKGLKWMGWINIYSGVPTYADDFKGRFVFSLEISASTAHLQINNLKNEDTATYFCARRKDYSGHCFDYWGQGTTLTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 212 - protein, 218 aa, synthetic construct
  source 1..218: note = Antibody LK-RB2-12 Light Chain
DIVLTQSPASLAVSPGQRATISCRASESVDTYGISFMHWYQQKPGQPPKLLIYRASNLESGIPARFSGSGSRTDFTLTINPVETDDVAIYYCQQSKKDPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 213 - protein, 447 aa, synthetic construct
  source 1..447: note = Antibody LK-RB2-13 Heavy Chain
QIQLVQSGPELKKPGETVKISCKASGYTFTTYGMSWVKQAPGKGLQWMGWINTYSGVPTFADDFKGRFAFSLDTSASTAYLQINNLKNEDTATYFCARRINWDGYAMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 214 - protein, 220 aa, synthetic construct
  source 1..220: note = Antibody LK-RB2-13 Light Chain
DSVMSQSPSSLAVSVGEKVTMSCKSSQSLLYSGNQKSYLAWYQQKPGQSPKLLIYWASTRESGVPDRFTGSGSGTDFTLTISSVKAEDLAVYYCQQYYSYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 215 - protein, 449 aa, synthetic construct
  source 1..449: note = Antibody LK-RB2-14 Heavy Chain
QVQLKQSGPGLVQPSQSLSITCTVSGFSLTSYGVHWVRQSPGKGLEWLGVIWRGGSTDYNAAFMSRLSITKDNSKSQVFFKMNSLQADDTAIYYCAKNEGDSTVVAWYFDVWGTGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 216 - protein, 214 aa, synthetic construct
  source 1..214: note = Antibody LK-RB2-14 Light Chain
DIQMTQTTSSLSASLGDRVTISCSASQGISNYLNWFQQKPDGTVKLLIYYTSSLHSGVPSRFSGSGSGTDYSLTISNLEPEDIATYYCQQYSKLPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 217 - protein, 447 aa, synthetic construct
  source 1..447: note = Antibody LK-RB2-15 Heavy Chain
QIQLVQSGPELKKPGETVKISCKASGYTFTTYGMSWVKQAPGKGLKWMGWINVYSGVPTYADDFKGRFAFSLEISASTAYLQINNLKDEDTATYFCARRKDYSGHCFDYWGQGTTLTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 218 - protein, 218 aa, synthetic construct
  source 1..218: note = Antibody LK-RB2-15 Light Chain
DIVLTQSPASLAVSLGQRATFSCRASESVDTYGISFMHWYQQKPGQPPKLLIYRASNLESGIPARFSGSGSRTDFTLTINPVETDDVATYYCQQSKKEPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 219 - protein, 444 aa, synthetic construct
  source 1..444: note = Antibody LK-RB2-16 Heavy Chain
QIQLVQSGPELKKPGETVKISCKASGYTFTTYGMGWVKQAPEKGLKWMGWINTNSGAPTYGDDFKGRFAFSLETSASTAYLQINNLKNEDTATYFCAHYGRPWFAYWGQGTLVTVSAASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 220 - protein, 214 aa, synthetic construct
  source 1..214: note = Antibody LK-RB2-16 Light Chain
DIQMTQSPASLSASVGETVTITCRASENIYSYLAWYHQKQGESPQLLVYNAKTLAEGVPSRFSGSGSGTQFSLKINSLQPEDFGSYYCQHHYNIPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 221 - protein, 447 aa, synthetic construct
  source 1..447: note = Antibody LK-RB2-17 Heavy Chain
QIQLVQSGPELKKPGETVKISCKASGYTFTNYGMSWVKQAPGKGLQWMGWINTYSGVPTFADDFRGRFAFSLDTSASTAYLQINSLKNEDTAAYFCARRINWDGYAMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 222 - protein, 220 aa, synthetic construct
  source 1..220: note = Antibody LK-RB2-17 Light Chain
DSVMSQSPSSLAVSVGEKVTLSCKSSQSLLYSGNQKSYLAWYQQKPGQSPKLLIYWASSRESGVPDRFTGSGSGTDFTLTISSVKAEDLAVYYCQQYYSFPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 223 - protein, 450 aa, synthetic construct
  source 1..450: note = Antibody LK-RB2-18 Heavy Chain
EVQLQQSGPELVKPGTSVKISCKASGYTFTDYYMNWVKQSHGKSLEWIGDINPNNGGTSYNQKFKGKATLTVDKSSSTAYMELRSLTSEDSAVFYCARSGTTVVGTYWYFDVWGTGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 224 - protein, 214 aa, synthetic construct
  source 1..214: note = Antibody LK-RB2-18 Light Chain
DIQMTQSPASLSVSVGETVTITCRASENIYSNLAWYQQKQGKSPQLLVYGATNLADGVPSRFSGSGSGTQYSLKINSLQSEDFGSYYCQHFWDTPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 225 - protein, 448 aa, synthetic construct
  source 1..448: note = Antibody LK-RB2-19 Heavy Chain
QVQLQQPGAELVKPGASVKLSCKASGYTFTSYWMHWVKQRPGQGLEWIGMIHPNSGGTNYNEKFKSKATLTVDKSSSTAYMQFSSLTSEDSAVYYCARCTVVALYYALDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 226 - protein, 213 aa, synthetic construct
  source 1..213: note = Antibody LK-RB2-19 Light Chain
QIVLSQSPAILSASPGEKVTMTCRATSRVSYIHWFQQKPGSSPKPWIYVTSNLASGVPARFSGSGSGTSYSLTISKLEAEDVATYYCQQWSTNPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 227 - protein, 449 aa, synthetic construct
  source 1..449: note = Antibody LK-RB2-20 Heavy Chain
QVQLKQPGAERVKPGASVKMSCKASGYTFTRYWITWVKQRPGQGLEWIGDIYPGSATTNYNEKFKSKATLTEDTSSSTAYMQLSSLTSEDSAVYYCARSGDYSNSNYAMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 228 - protein, 214 aa, synthetic construct
  source 1..214: note = Antibody LK-RB2-20 Light Chain
NIEMTQSPKSMSMAVGERVTLSCKASENVGTYVSWYQQKPEQSPKLLIYGASNRNTGVPDRFTGSGSATDFTLTISSVQAEDLADYHCGQSYSYPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 229 - protein, 448 aa, synthetic construct
  source 1..448: note = Antibody LK-RB2-21 Heavy Chain
EVQLVESGGDLVKPGGSLKLSCAASGFTFSSYGMSWVRQTPDKRLEWVATISSGGSYTYYPDSLKGRFTISRDNAKSTLYLQMSSLKSEDTAKYYCARPYYDYIFYAIDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 230 - protein, 214 aa, synthetic construct
  source 1..214: note = Antibody LK-RB2-21 Light Chain
DIQMNQSPSSLSASLGDTITITCHASQNINVWLSWYQQKPGNIPKLLIYKASNLHTGVPSRFSGSGSGTGFTLTISSLQPEDIATYYCQQGQSYPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 231 - protein, 447 aa, synthetic construct
  source 1..447: note = Antibody LK-RB2-22 Heavy Chain
QIQLVQSGPELKKTGETVKISCKASGYTFTTYGMSWVKQAPGKGLKWMGWINIYSGVPTYADDFKGRFAFSLETSASTAYLQINNLKNEDTATYFCARRADYDGYYFDYWGQGTTLTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 232 - protein, 214 aa, synthetic construct
  source 1..214: note = Antibody LK-RB2-22 Light Chain
NIVMTQTPKFLLVSAGDRVTITCRASQSVTNDVAWYQQRPGQSLKLLIYYASNRYTGVPDRFTGSGYGTDFTFTISTVQAEDLAVYFCQQGYSSPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 233 - protein, 447 aa, synthetic construct
  source 1..447: note = Antibody LK-RB2-23 Heavy Chain
QIQLVQSGPELKKPGETVKISCKASGYTFTTYGMSWVKQAPGKGLKWMGWINIYSGVPTYADDFKGRFAFSLETSASTAYLQINNLKNEDTATYFCARRGDYDGYYFDYWGQGTTLTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 234 - protein, 214 aa, synthetic construct
  source 1..214: note = Antibody LK-RB2-23 Light Chain
NIVLIQSAATLSVTPGDSVSLSCRASQSLSNGLHWYQQKSHESPRLLIKYGSQSISGIPSRFSGSGSGTDFTLSINSVETEDFGMYFCQQSDSWPHTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 235 - protein, 447 aa, synthetic construct
  source 1..447: note = Antibody LK-RB2-24 Heavy Chain
QIQLVQSGPEVKKPGETVKISCKASGYTFTTYGMSWVKQAPGKGLKWMGWLNIYSGVPAYADDFKGRFAFSLESSASTAYLQINNLKNEDTATYFCARRENYDGYGMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 236 - protein, 214 aa, synthetic construct
  source 1..214: note = Antibody LK-RB2-24 Light Chain
DIVMTQSPATLSVTPGDRVSLSCRASQSISDYLHWYQQKSHESPRLLIKYASQSISGIPSRFSGSGSGSDFTLSINSVEPEDVGVYYCQNGHSFPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 237 - protein, 445 aa, synthetic construct
  source 1..445: note = Antibody LK-RB2-25 Heavy Chain
QVQLKESGPGLVAPSQSLSITCTVSGFSLTSYGVHWVRQPPGKGLEWLGVIWAGGSTKYNSALMSRLSINKDNSKSQVFLKMNSLQTDDTAMYYCARDQMVRSAMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 238 - protein, 214 aa, synthetic construct
  source 1..214: note = Antibody LK-RB2-25 Light Chain
DIQMIQSPSSMFASLGDRVSLSCRASQGIRGNLDWYQQKPGGTIKLLIYSTSNLNSAVPSRFSGSGSGSDYSLTISNLESEDFADYYCLQRNASPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 239 - protein, 445 aa, synthetic construct
  source 1..445: note = Antibody LK-RB2-26 Heavy Chain
QVQLKESGPGLVAPSQSLSITCTVSGFSLTSYGVHWVRQPPGKGLEWLGVIWAGGSTDYNAAFISRLNISKDISESQVFFRMNSLQTDDTAMYYCARDQMVRSAMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 240 - protein, 214 aa, synthetic construct; identical to SEQ ID NO: 238
  source 1..214: note = Antibody LK-RB2-26 Light Chain

SEQ ID NO: 241 - protein, 441 aa, synthetic construct
  source 1..441: note = Antibody LK-RB2-27 Heavy Chain
DVKLVESGGGLVKPGGSLKLSCAASGFTFSSYSMSWVRQTPEKRLEWVATISTGGSYTYYPDSVKGRFTISRDNAKNTLYLQMSSLKSEDTAMFYCTRGSMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 242 - protein, 214 aa, synthetic construct
  source 1..214: note = Antibody LK-RB2-27 Light Chain
DILMTQSPSSMSVSLGDTVSITCHASQDISSNIGWLQQKPGNSFKGLIYHGTNLEDGVPSRFSGSGSGADYSLTISSLESEDFADYYCVQYAQFPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 243 - protein, 441 aa, synthetic construct
  source 1..441: note = Antibody LK-RB2-28 Heavy Chain
DVKLVESGGGLVKPGGSLKLSCAASGFTFSSYSMSWVRQTPEKRLEWVATISTGGSYTYYPDSMKGRFTISRDNAKNTLYLQMSSLKSEDTAMFYCTRGSMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 244 - protein, 214 aa, synthetic construct
  source 1..214: note = Antibody LK-RB2-28 Light Chain
DILMTQSPSSMSVSLGDTVSIACHASQDISSNIGWLQQKPGNSFKGLIYHGTNLEDGVPSRFSGSGSGADYSLTISSLESEDFADYYCVQYAQFPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 245 - protein, 449 aa, synthetic construct
  source 1..449: note = Antibody LK-RB2-29 Heavy Chain
QVQLQQSGAELARPGASVKLSCKASGYTFTSYWMQWVKQRPGQGLEWIGAIYPGDGDTRYTQKFKGKATLTADKSSSTAYMQLSSLASEDSAVYYCARGYYGSSYADYFDYWGQGTTLTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 246 - protein, 214 aa, synthetic construct
  source 1..214: note = Antibody LK-RB2-29 Light Chain
DIQMTQSPASLSASVGETVTITCRASGNIHNYLAWYQQKQGKSPQLLVYNAKTLADGVPSRFSGSGSGTQYSLKINSLQPEDFGSYYCQHFWSTPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 247 - protein, 446 aa, synthetic construct
  source 1..446: note = Antibody LK-RB2-30 Heavy Chain
EVQLVESGGGLVQPGGSLKLSCAASGFTFNTDGMSWVRQTPDKRLELVAIINSNGGSTYYPDSVKGRFTISRDNAKNTLYLQMSSLKSEDTAMYYCAREGSYGSWFAYWGQGTLVTVSAASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 248 - protein, 214 aa, synthetic construct
  source 1..214: note = Antibody LK-RB2-30 Light Chain
DIQMTQSPASLSVSVGETVTITCRASENINNNLAWYHQKQGKSPQLLVYAATNLADGVPSRFSGSGSGTQYSLKINSLQSEDFGSYYCQHFWGTPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 249 - protein, 442 aa, synthetic construct
  source 1..442: note = Antibody LK-RB2-31 Heavy Chain
QVQLQQPGAELVKPGASMKLSCKASGYTFTTYYMYWVKQRPGLGLEWIGGINPSNGGTTFNEKFKNKATLTVDKSSSTAYMQLRSLTSEDSAVYYCTRWRFFDVWGAGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 250 - protein, 214 aa, synthetic construct
  source 1..214: note = Antibody LK-RB2-31 Light Chain
SIVMTQTPEFLLVSAGDRVTITCKASQSVNNDVAWFQQKPGQSPKLLIYYASNRYTGVPDRFTGSGYGTDFTFTISTVQAEDLAIYFCQQDYSSPPTFGGATKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 251 - protein, 444 aa, synthetic construct
  source 1..444: note = Antibody LK-RB2-32 Heavy Chain
DVQLQESGPGLVKPSQSLSLTCSVTGYSITSGYYWNWIRQFPGNKLEWMGYISFDGSNHYNPSLRNRISITRDTFKNQFFLKLNSVTTEDTATFYCTRRDYNWFAYWGQGTLVTVSAASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 252 - protein, 214 aa, synthetic construct
  source 1..214: note = Antibody LK-RB2-32 Light Chain
DIVLTQSPVTLSVTPGDSVSLSCRASQNINKNLHWYQQRSHESPRLLIKYVSQSISGIPSRFSGSGSGTDFTLSINSVETEDFGMYFCQQSNSWPLTFGAGTKLELRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 253 - protein, 448 aa, synthetic construct
  source 1..448: note = Antibody LK-RB2-33 Heavy Chain
EVQLQQSGVELVRPGALVKLSCKASGFNIKDYYMHWVKQRPEQGLEWIGWIDPEKGDTIYGPKFQGKASITADTSSNTAYLQLSSLTSEDTAVYYCSRGPNYGSSYAFAYWGQGTLVTVSAASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 254 - protein, 214 aa, synthetic construct
  source 1..214: note = Antibody LK-RB2-33 Light Chain
DVVMTQSHKFMSTSVGDRVTITCKASQDVSNAVAWYQQKPGQSPKLLIYSASYRYTGVPDRFTGSGSGTDFTFTISSMQAEDLAVYYCQQHYSLPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 255 - protein, 441 aa, synthetic construct
  source 1..441: note = Antibody LK-RB2-34 Heavy Chain
DVKVVESGGGLVKPGGSLKLSCAASGFTFSSYSMSWVRQTPEKRLEWVATISTGSSYTYYSDSVKGRFTISRDNAKNTLYLQMSSLKSEDTAMFYCTRGSMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 256 - protein, 214 aa, synthetic construct
  source 1..214: note = Antibody LK-RB2-34 Light Chain
DILMTQSPSSMSVSLGDTVSITCHASQDISSNIGWLQQKPGNSFKGLIYHGINLEDGVPSRFSGSGSGADYSLTISSLESEDFADYYCVQYAQFPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 257 - protein, 446 aa, synthetic construct
  source 1..446: note = Antibody LK-RB2-35 Heavy Chain
EVQLQQSGPELVKPGASVKISCKASGYTFTDYNMHWVKQSHGKSLEWIGSIYPYNGGSDYNQKFKSKATLTVDNSSSTAYMELRSLTSEDSAVYYCASELTGLYAMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 258 - protein, 214 aa, synthetic construct
  source 1..214: note = Antibody LK-RB2-35 Light Chain
DIVMTQSPATLSVTPGDRVSLSCRASQSISDYLHWYQQKSHESPRLLIKYASQSISGIPSRFSGSGSGSDFTLSINSVEPEDVGVYYCQTGHSFPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 259 - protein, 447 aa, synthetic construct
  source 1..447: note = Antibody LK-RB2-36 Heavy Chain
QVQLQQSGAELVRPGASVKMSCKASGYTFTNYWIGWAKQRPGHGLEWIGDIYPGGTYTGYNEKFKGKATLTADKSSSTAYMQFSSLTSEDSAIYYCARRMDYDGYAMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 260 - protein, 214 aa, synthetic construct
  source 1..214: note = Antibody LK-RB2-36 Light Chain
DIVLTQSPASLSVTPGDSVSLSCRASQSISNNLHWYQQKSHESPRLLIKYASQSISGIPSRFSGSGSGTDFTLSINSVETEDFGMYFCQQSNSWPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 261 - protein, 447 aa, synthetic construct
  source 1..447: note = Antibody LK-RB2-37 Heavy Chain
QIQLVQSGPEVKKPGETVKISCKASGYTFTTYGMSWVKQAPGKGLKWMGWINTYSGVPTYADDFKGRFAFSLETSVSTAYLQINNLKNEDTSTYFCARRRDYDGYAMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 262 - protein, 220 aa, synthetic construct
  source 1..220: note = Antibody LK-RB2-37 Light Chain
DIVMSQSPSSLAVSVGEKVTMSCKSSQSLLYSGNQKNYLAWYQQKPGQSPKLLIYWASARESGVPDRFTGSGSGTDFTLTISSVKAEDLAVYYCQQYYTYPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 263 - protein, 445 aa, synthetic construct
  source 1..445: note = Antibody LK-RB2-38 Heavy Chain
EVKLVESGGGLVQPGGSLKLSCAASGFSFSDYYMYWVRQTPEKRLEWVAYISNGGGNTYYPDTVKGRFTISRDNAKNSLYLQMSRLKSEDTGMYYCARDREDWYFDVWGTGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 264 - protein, 214 aa, synthetic construct
  source 1..214: note = Antibody LK-RB2-38 Light Chain
DIVMTQSHTFMSTSVGDRVSITCKASQDVSTAVAWYQQKPGQSPKLLIYWASTRHTGVPDRFTGSGSGTDYTLTISSVQAEDLALYYCQQHYSTPRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 265 - protein, 445 aa, synthetic construct
  source 1..445: note = Antibody LK-RB2-39 Heavy Chain
DVKLVESGGGSVQPGGSLKLSCAASGFTFSDYYMYWVRQTPEKRLEWVAYISNGGGNTYYPDTVKGRFTISRDNAKNTLYLQMSRLKSEDTAMYYCARSNWAWYFDVWGTGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 266 - protein, 220 aa, synthetic construct
  source 1..220: note = Antibody LK-RB2-39 Light Chain
DIVMTQSPSSLAMSVGQKVTMSCKSSQSLLHSSNQKNYLAWYQQKPGQSPKLLVSFASTRESGVPDRFIGSGSGTDFTLTINSVQAEDLADYFCQQHYSTPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 267 - protein, 450 aa, synthetic construct
  source 1..450: note = Antibody LK-RB2-40 Heavy Chain
DVQLQESGPGLVKPSQSLSLTCSVTGYSITSGYYWNWIRQFPGNKLEWMAYISYDGSNNYNPSLKNRISITRDTSKNQFFLKLNSVTTEDTATYYCAREEGTAQATLGYFDYWGQGTTLTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 268 - protein, 214 aa, synthetic construct
  source 1..214: note = Antibody LK-RB2-40 Light Chain
DIQMTQSPASLSVSVGETVTITCRASENIYSNLAWYQQKQGKSPQLLVYAATNLADGVPSRFSGSGSGTQYSLKINSLQSEDFGSYYCQHFWDTPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 269 - protein, 448 aa, synthetic construct
  source 1..448: note = Antibody LK-RB2-41 Heavy Chain
EVQLQLSGPELVKPGASVKMSCKASGYTFTDYNMHWVKQSHGKSLEWIGYINPNNGGTTYNQKFKGKATLTVNKSSSTAYMELRSLTSEDSAVYYCARAPYYGSSYLLAYWGQGTLVTVSAASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 270 - protein, 214 aa, synthetic construct
  source 1..214: note = Antibody LK-RB2-41 Light Chain
DIVMTQSHKFMSTSVGDRVSITCKASQDVSTAVAWYQQKPGQSPKLLIYSASYRYTGVPDRFTGSGSGTDFTFTISSVQAEDLAIYYCQQHYSIPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 271 - protein, 445 aa, synthetic construct
  source 1..445: note = Antibody LK-RB2-42 Heavy Chain
EFQLQQSGPELVKPGASVKISCKASGYSFTDYNMNWVKQSNGKSLDWIGIINPKYGTTAYNQKLRDKATLTVDQSSSTAYMQLNSLTSEDSAVYYCASLNWDEGFAYWGQGTLVTVSPASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 272 - protein, 219 aa, synthetic construct
  source 1..219: note = Antibody LK-RB2-42 Light Chain
DVLMTQTPLSLPVSLGDQASISCRSSQSIVHSNGNTYLEWYLQKPGQPPKLLIYKVSNRFSGVPERFSGSGSGTDFTLKINRVEAEDLGVYYCFQGSLVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 273 - protein, 441 aa, synthetic construct
  source 1..441: note = Antibody LK-RB2-43 Heavy Chain
EVQLVESGGDLVKPGGSLKLSCAASGFTFSSYGMSWVRQTPDKRLEWVATISGGGTYTYYPDSVKGRFTISRDNAKNTLYLQMSSLKSEDTAMYYCASGAMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 274 - protein, 213 aa, synthetic construct
  source 1..213: note = Antibody LK-RB2-43 Light Chain
QIVLTQSPAILSASPGEKVTMTCSANSSVSSMYKYQQKPGSSPKPWIYGTSNLASGVPARFSGSGSGTSYSLTISSMEAADAATYYCQQYHSHPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 275 - protein, 441 aa, synthetic construct; identical to SEQ ID NO: 273
  source 1..441: note = Antibody LK-RB2-44 Heavy Chain

SEQ ID NO: 276 - protein, 219 aa, synthetic construct
  source 1..219: note = Antibody LK-RB2-44 Light Chain
DVVMTQTPLTLSVTIGQPASISCKSSQSLLDIDGKTFLNWLLQRPGQSPKRLIYLVSKLDSGVPDRFTGSGSGTDFTLKISRVEAEDLGVYYCWQGTHFPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 277 - protein, 447 aa, synthetic construct
  source 1..447: note = Antibody LK-RB2-45 Heavy Chain
QIQLVQSGPELKKPGETVKISCKASGYTFTTYGMSWVKQTPGKGLQWMGWINVYSGVPTYADDFKGRFAFSLEASASTAYLQINNLKNEDTASYFCARRGDYDGHYFDYWGQGTTLTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 278 - protein, 214 aa, synthetic construct
  source 1..214: note = Antibody LK-RB2-45 Light Chain
DIVMTQSPATLSVTPGDRVSLSCRASQSISDYLNWYQQKSHESPRLLIKYVSQSISGIPSRFSGSGSGSDFTLSINSVEPEDIGVYYCQNGHNFPLTFGGGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 279 - protein, 444 aa, synthetic construct
  source 1..444: note = Antibody LK-RB2-46 Heavy Chain
QVQMQQSDAELVKPGASVKISCKVSGYTFTDQIIHWMKQRPEQGLEWIGYIYPRDDSIMYNEKFKGKATLTADKSSSTANMQLNSLTSEDSAVYFCAYDAYWYFDVWGTGTTVIVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 280 - protein, 214 aa, synthetic construct
  source 1..214: note = Antibody LK-RB2-46 Light Chain
DIVMTQSHKFMSASVGDRVSITCKASQDVNTAVAWYRQDPGQSPKLLIYWASTRHTGVPDRFTGSGSGTDYTLTINNFQAEDLALYYCQQHYSPPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 281 - protein, 446 aa, synthetic construct
  source 1..446: note = Antibody LK-RB2-47 Heavy Chain
QVQLKQSGPGLVQPSQSLSITCTVSGFSLISYGVHWVRQSPGKGLEWLGVIWSGGSTDYNAAFISRLSISKDNSKSQVFFKMNSLQADDTAIYYCARSFHYHGSTFDYWGQGTTLTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 282 - protein, 214 aa, synthetic construct
  source 1..214: note = Antibody LK-RB2-47 Light Chain
DIQMTQTTSSLSASLGDRVIISCRASQDINNYLNWYQQKPDGTVKLLIYFTSRLHSGVPSSFSGSGSGTDYSLTINNLEQEDIATYFCQQVNTLPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 283 - protein, 122 aa, synthetic construct
  source 1..122: note = Antibody LK-RB1-1 VH
QIQLVQSGPELKKPGETVKISCKASGYSFTNFGMSWVKQAPGKALKWMGWINTYSGVPTYADDFKGRFAFSLETSANTAYLQINNLKNEDSATYFCARRRRTFDGYLCFDYWGQGATLTVSS

SEQ ID NO: 284 - protein, 115 aa, synthetic construct
  source 1..115: note = Antibody LK-RB1-1 VL
QLVLTQSSSASFSLGASAKLTCTLSSQHSTYSIDWYQQQPLKPPKYVMELKTDGSHRTGDGIPDRFSGSSSGADRYLSISNIQPEDEAIYICGVGDTIKEQFVYVFGGGTKVTVL

SEQ ID NO: 285 - protein, 122 aa, synthetic construct
  source 1..122: note = Antibody LK-RB1-2 VH
QIQLVQSGPELKKPGETVKISCKASGYTFTTYGVSWVKQAPGKGLKWMGWINTYSGVSTYSDDFKGRFAFSLETSASTAYLQIINLKNEDAATYFCSRRRRTYDGYLSFDYWGQGTTLTVSS

SEQ ID NO: 286 - protein, 107 aa, synthetic construct
  source 1..107: note = Antibody LK-RB1-2 VL
DIQMTQSPGSLSASVGETVTITCGASENIYGALNWFQRKPGKSPQLLIYGATNLADGMSSRFSGSGSGRQYSLQISSLRPDDVATYYCQNVLTTPWTFGGGTKVEIK

SEQ ID NO: 287 - protein, 119 aa, synthetic construct
  source 1..119: note = Antibody LK-RB1-3 VH
QVQLQQPGAELVKPGTSVNLSCKASGYTFTSYWIHWVKQRPGRGLEWIGRINPYSSGTRYNEKFKNKATLTVDKPSSTAYMQLTSLTSEDSAVYYCARFDGAPDYFDFWGQGTTLTVSS

SEQ ID NO: 288 - protein, 107 aa, synthetic construct
  source 1..107: note = Antibody LK-RB1-3 VL
DIVMTQSQKFMPTSVGDRVNVTCKASQNVGINVAWYQQKLGQSPKALIYSASYRYSGVPDRFTGSGSGTDFTLTINSVQSEDLADYFCQQYNNYPFTFGSGTKLEIK

SEQ ID NO: 289 - protein, 119 aa, synthetic construct
  source 1..119: note = Antibody LK-RB1-4 VH
QVQLQQPGAELVKPGASVKLPCKTSGYTFTSYWMHWVKQRPGRGLEWIGRIDPHYSGTKYNEKFKNKATMTVDTPSSTAYMQLSSLTSEDSAVYYCARFDGDPYYFDYWGPGTTLTVSS

SEQ ID NO: 290 - protein, 107 aa, synthetic construct
  source 1..107: note = Antibody LK-RB1-4 VL
DIVMTQSQKFMSTSVGDRVSVTCKASQNVGTNVAWYKQKAGQSPKAVIYSASYRYSGVPDRFTGSGSGTDFTLSISNVQSEDLAEYFCQQYNNNPFTFGSGTKLEIK

SEQ ID NO: 291 - protein, 120 aa, synthetic construct
  source 1..120: note = Antibody LK-RB1-5 VH
QVQLQQPGAELVKPGASVKLSCKASGYTFTDFWIHWVKQRPGQGLEWIGMIHPNSGSTNFNEKFRAKATLTVDKSSSTAYMRLSSLTSEDSAVYYCARSGDGYSNPMDYWGQGTSVTVSS

SEQ ID NO: 292 - protein, 106 aa, synthetic construct
  source 1..106: note = Antibody LK-RB1-5 VL
QIVLSQSPAILSASPGEKVTLTCRASSSVSYMHWYQQKPGSSPKPWIYATSNLASGVPVRFSGSGSGTSYSLTISRMQTEDAATYYCQQWRRSPYTFGGGTKLEIK

SEQ ID NO: 293 - protein, 122 aa, synthetic construct
  source 1..122: note = Antibody LK-RB1-6 VH
QIQLVQSGPELKKPGETVKISCKASGYTFTTYGMSWVKQAPGKDLKWMGWINTFSGVPACADDFQGRFDFSAEPSDSTAHLQINNLKNEDTATYFCARRSYYGSRGWYFDVWGTGTTVTVSS

SEQ ID NO: 294 - protein, 107 aa, synthetic construct
  source 1..107: note = Antibody LK-RB1-6 VL
DIVMTQSHKFMSTSVGDRVSITCTASEDVNTAVAWYQQKPGQSPKLLIYWASTRHTGVPDRFTGSGSGTDYTLTISSVQTEDLALYYCQQHYNTPYTFGGGTKLEIK

SEQ ID NO: 295 - protein, 122 aa, synthetic construct; identical to SEQ ID NO: 293
  source 1..122: note = Antibody LK-RB1-7 VH

SEQ ID NO: 296 - protein, 107 aa, synthetic construct
  source 1..107: note = Antibody LK-RB1-7 VL
DVQMIQSPSSLSASLGDIVTMTCQASQDTTINLNWFQQKPGKVPKLLIYGASNLEDGVPSRFSGSSFGTDFTLTISSLEDEDMATYFCLQHTYLPRTFGGGTKLEIR

SEQ ID NO: 297 - protein, 120 aa, synthetic construct
  source 1..120: note = Antibody LK-RB1-8 VH
QVQLQQSGAELVRPGTSVKVSCKASGYAFSNYLIEWVKQRPGQGLEWIGVINPGSDGSTYNEKFKGKATLTADKSSNTAYMQLSSLTSEDSAVYFCARGDYGRSYGMDYWGQGTSVTVSS

SEQ ID NO: 298 - protein, 107 aa, synthetic construct
  source 1..107: note = Antibody LK-RB1-8 VL
DIVMTQSQKFMSTTVGDRVSITCKASQNVGTAVVWYQQKPGQSPKLLIYSASNRYTGVPDRFTGSGSGTDFTLTISNMQSEDLADYFCQQYSSYMYTFGGGTKLEIK

SEQ ID NO: 299 - protein, 122 aa, synthetic construct
  source 1..122: note = Antibody LK-RB1-9 VH
QIQLVQSGPELKKPGETVKISCTASGYTFTTYGMSWVKQAPGKGLKWMGWINTYSGVPTYADDFKGRFAFSLETSASTAYLEINNVKNEDTATYFCARRRRTYHGYLCFDYWGQGTTLTVSS

SEQ ID NO: 300 - protein, 107 aa, synthetic construct
  source 1..107: note = Antibody LK-RB1-9 VL
DIQMTQSPVSLSASVGETVTITCETSENIYGALNWYQRKQGKSPQLLIYGATNLADGMSSRFSGSGSGRQYSLEISSLHPDDVASYYCQNVLNTPWTFGGGTKLEIK

SEQ ID NO: 301 - protein, 118 aa, synthetic construct
  source 1..118: note = Antibody LK-RB1-10 VH
QVTLKESGPGILKPSQTLSLTCSFSGFSLSTSGTGVGWIRQPSGKGLEWLAHIWWNDDKYYKPSLKRQLTISKDTSRNQVFLKITSVDTADTATYYCTRTELGRGDYWGQGTSVTVSS

SEQ ID NO: 302 - protein, 107 aa, synthetic construct
  source 1..107: note = Antibody LK-RB1-10 VL
DILLTQSPAILSVSPGERVSFSCRASQSIGTNIHWYQQRMNGSPRLIIKYASELISGIPSRFSGSGSGTDFTLSINSVESEDIADYYCQQSYGWPYTFGGGTKLETK

SEQ ID NO: 303 - protein, 122 aa, synthetic construct
  source 1..122: note = Antibody LK-RB1-11 VH
QVTLKESGPGILQPSQTLSLTCSFSGFSLKTSGMSVGWIRQPSGKGLEWLAHIWWNDDKYYNPVLKGRLTISKDTSNNLVFLKIASVVSTDTATYYCVRYGNSHGYWYFDVWGTGTTVTVSS

SEQ ID NO: 304 - protein, 111 aa, synthetic construct
  source 1..111: note = Antibody LK-RB1-11 VL
NIVLTQSPASLAVSLGQRATISCRASESVDSYGNNFIHWYQQKPGQPPKVLIYYASNLEAGVPARFSGSGSRTDFTLTIDPVEAEDAATYYCQQNNEDPYTFGGGTKLEIE

SEQ ID NO: 305 - protein, 120 aa, synthetic construct
  source 1..120: note = Antibody LK-RB1-12 VH
QVQLQQSGTELVRPGSSVMISCKASGYAFSTYWMDWVKQRPGQGLEWIGQIYPGDGATNYNGKFKGKATLTADKSSITAYMHLSSLTSEDSAVYFCLRSRDYDGWFFDVWGAGTTVTVSS

SEQ ID NO: 306 - protein, 107 aa, synthetic construct
  source 1..107: note = Antibody LK-RB1-12 VL
DIVMTQSHKFMSTSIGDRVSITCKASQDVSSAVAWYQQKPGQSPKLLIYWASTRHTGVPDRFTGSGSGTDYTLTFSSVQAEDLALYYCQQHYSTPWTFGGGTKLEIK

SEQ ID NO: 307 - protein, 118 aa, synthetic construct
  source 1..118: note = Antibody LK-RB1-13 VH
QVTLKESGPGIVKPSQTLSLTCSFSGFSLSISGTGIGWIRQPSGKGLEWLAHIWWDDDKYYNPSLKSQVTISKDTSTKQVFLNITSVDTADTATYYCTRTELGRGDFWGQGTSVTVSS

SEQ ID NO: 308 - protein, 107 aa, synthetic construct
  source 1..107: note = Antibody LK-RB1-13 VL
DILLTQSPAILSVSPGDRVSFSCRASQSTGTNIHWYQRRTNGSPRLLIKYGSESISGIPSRFSGSGTGTDFTLTINSVESEDIADYYCQQSYSWPYTFGGGTKLEIK

SEQ ID NO: 309 - protein, 118 aa, synthetic construct
  source 1..118: note = Antibody LK-RB1-14 VH
QVTLKESGPGILKPSQTLSLTCSFSGFSLSTSGMGVGWIRQPSGKGLEWLAHIWWDDDKYYNPSLKSQLTISKDTSRNQVFLKITSVDTADTATYYCARTELGRGDSWGQGTSVTVSS

SEQ ID NO: 310 - protein, 107 aa, synthetic construct
  source 1..107: note = Antibody LK-RB1-14 VL
DILLTQSPAILSVSPGERVSFSCRASQSIGTSIHWYQQRTNGSPRLLIKYASESISGIPSRFSGSGSGTDFTLSINSVESEDIADYYCQQSYSWPYTFGGGTKLEIK

SEQ ID NO: 311 - protein, 118 aa, synthetic construct; identical to SEQ ID NO: 307
  source 1..118: note = Antibody LK-RB1-15 VH

SEQ ID NO: 312 - protein, 107 aa, synthetic construct
  source 1..107: note = Antibody LK-RB1-15 VL
DILLTQSPAILSVSPGDRVSFSCRASQSIGTNIHWYQRRTNGSPRLLIKYGSESISGIPSRFSGSGTGTDFTLTINSVESEDIADYYCQQSYSWPYTFGGGTKLEIK

SEQ ID NO: 313 - protein, 118 aa, synthetic construct
  source 1..118: note = Antibody LK-RB1-16 VH
QVQLKESGPGLVAPSQSLSITCTVSGFSLISNAISWVRQPPGKGLEWLGVIWGDGNTIYHSALRSRLTISKDNSKSQVFLKLNSLQIDDTATYYCAKANWDGYYFDYWGQGTTLTVSS

SEQ ID NO: 314 - protein, 107 aa, synthetic construct
  source 1..107: note = Antibody LK-RB1-16 VL
DIQMNQSPSSLSASLGDTITITCHASQSVDFWLNWYQQKPGNIPKLLISKASNLHTGVPSRFSGSGSGTTFTLTISSLQPEDIATYYCQQGQSYPLTFGSGTKLEIK

SEQ ID NO: 315 - protein, 119 aa, synthetic construct
  source 1..119: note = Antibody LK-RB1-17 VH
QVQLQQPGAELVKPGASVKLSCEASGYTFTNYWIHWVKQRPGRGLEWIGRIAPNSGGTKYNEKFKTRATLTVDKASTTAYMQLSSLTSEDSAVYYCARLDGSPYYFDYWGQGTTLTVSS

SEQ ID NO: 316 - protein, 107 aa, synthetic construct
  source 1..107: note = Antibody LK-RB1-17 VL
DIVMTQSQKFMSTSIGDRVSVTCKASQNVDTNVAWYQQKPGQSPKALIYSASYRYSGIPDRFTGSGSGTDFTLTISNVQSEDLAEYFCQQYNSYPYTFGGGTKLEIK

SEQ ID NO: 317 - protein, 118 aa, synthetic construct
  source 1..118: note = Antibody LK-RB1-18 VH
QVQLKESGPGLVAPSQSLSITCTVSGFSLTNSGVTWIRQPPGKGLEWLGVIWGDGSTNYHSALISRLSISKDNSKSQVFLKLNSLQTDDTATYYCAKPHWDYYNMDYWGQGTSVTVSS

SEQ ID NO: 318 - protein, 107 aa, synthetic construct
  source 1..107: note = Antibody LK-RB1-18 VL
DIQMTQTPSSLSASLGDRVTISCRASQDISNYLNWYQQKPDGTVKLLIYFTSRLHSGVPSRFSGSGSGTDYSLTISNLEQEDFATYFCQQGITLPLTFGAGTKLELK

SEQ ID NO: 319 - protein, 118 aa, synthetic construct
  source 1..118: note = Antibody LK-RB1-19 VH
QVQLKESGPGLVAPSQNLSITCTVSGFSLTSYGVSWVRQPPGKGLEWLGIIWGDGNTDYHSALISRLSIRKDNSKSQVFLKLNSLKSDDTATYYCAKPHWEYYTMDYWGQGTSVTVSS

SEQ ID NO: 320 - protein, 107 aa, synthetic construct
  source 1..107: note = Antibody LK-RB1-19 VL
DIQMTQTTSSLSASLGDRVTISCRASQDISNFLNWYQQKPDGTVKLLIYSTSRLHSGVPSRFSGSGSGTDYSLTIANLEQEDVATYFCQQGNTLPLTFGAGTKLELK

SEQ ID NO: 321 - protein, 118 aa, synthetic construct
  source 1..118: note = Antibody LK-RB1-20 VH
QVQLKESGPGLVAPSQSLSITCTVSGFSLNSNGVSWVRQPPGKGLEWLGLIWGDGSTNYHSALISRLSIRKDNSKSQVFLKLNSLQNDDTATYYCAKPNWDGYHFDYWGQGTTLIVSS

SEQ ID NO: 322 - protein, 107 aa, synthetic construct
  source 1..107: note = Antibody LK-RB1-20 VL
DIQMNQSPSSLSASLGDTITITCHASQNIDFWLSWYQQKPGNIPKLLIYKASNLHTGVPSRFSGSGSGTGFTLTISSLQPEDIATYYCQQGQSYPLTFGAGTKLELK

SEQ ID NO: 323 - protein, 119 aa, synthetic construct
  source 1..119: note = Antibody LK-RB1-21 VH
QVQLQQPGAELVKPGASVKMSCKASGYIFTDYWTTWVKQRPGQGLEWIGDIYPVSGSTNYNEKFKNKATLTVDTSSSTAYMQLSSLTSEDSAVYYCARALGRRYYFDYWGQGTTLTVSS

SEQ ID NO: 324 - protein, 107 aa, synthetic construct
  source 1..107: note = Antibody LK-RB1-21 VL
DIVMTQSHKFMSTSIGDRVSITCKASQDVSTAVAWCQQKPGQSPKSLIYSASYRYTGIPDRFTGSGSGTDFTFTISSVQAEDLAVYYCQQHYTNPVTFGTGTKLELK

SEQ ID NO: 325 - protein, 118 aa, synthetic construct
  source 1..118: note = Antibody LK-RB1-22 VH
QVQLKESGPGLVAPSQSLSITCTVSGFSLTSYGVSWVRQPPGKGLEWLGIIWGDGNTNYHSALTSRLSISKDNSKSQVFLKLNSLRTDDTATYYCAKPHWDHYSMDYWGQGTSVTVSS

SEQ ID NO: 326 - protein, 107 aa, synthetic construct
  source 1..107: note = Antibody LK-RB1-22 VL
DIQMTQTTSSLSASLGDRVTISCRASQDISNFLNWYQQKPDGTVKLLIYYTSRLHSGAPSRFSGSGSGTDYSLTISNLDQEDIATYFCQQGNTLPLTFGAGTKLELK

SEQ ID NO: 327 - protein, 117 aa, synthetic construct
  source 1..117: note = Antibody LK-RB1-23 VH
QVQLQQTGAELVRPGSSVKLSCKASGYTFTNYWMDWVKQRPGQGLEWIGNIHPFGNETHYNQKFKDKATLTVDKSSSTAYMQLSSLTSEDSAVYYCARGLLLYFDVWGTGTTVTVSS

SEQ ID NO: 328 - protein, 107 aa, synthetic construct
  source 1..107: note = Antibody LK-RB1-23 VL
DIQMTQSPASQSVSVGETVTITCRASENIYSNLAWYQQKQGKSPQLLVYGATNLADGVSLRFSGSGSGTQYSLKINSLQSEDFGSYYCQHFWGIPWTFGGGTKLEIK

SEQ ID NO: 329 - protein, 114 aa, synthetic construct
  source 1..114: note = Antibody LK-RB1-24 VH
QVQLQQPGAELVNPGTSVNLSCKASGYTFTGYWMHWVKQRPGQGLEWIGMIHPNNGGTIYNERFNGKATLTVDRSSTTAYIQLSSLTSEDSAVYYCAYWDLDYWGQGTTLTVSS

SEQ ID NO: 330 - protein, 112 aa, synthetic construct
  source 1..112: note = Antibody LK-RB1-24 VL
DVMMTQTPLTLSVTIGQPASISCKSSQSLLNSDGKTYLNWLLQRPGQSPKRLIYLVSKLGSGVPDRFTGSGSGTDFTLKISRVEAEDLGVYYCWQGTHFPWTFGGGAKLEIK

SEQ ID NO: 331 - protein, 121 aa, synthetic construct
  source 1..121: note = Antibody LK-RB1-25 VH
QIQLVQSGPELKKPGETVKISCKASGYTFTSYGMNWMKQAPGEGLKWMGWINTYSGVPTYADDFKGRFAFSLETSATTVYLQINNLKNEDKATYFCARRGRDSVVGHFDVWGTGTTVTVSS

SEQ ID NO: 332 - protein, 107 aa, synthetic construct
  source 1..107: note = Antibody LK-RB1-25 VL
DIVMTQSHKFMSTSVGDRVSITCKASQDVNTAVAWYQQKPGQSPKLLIYWASTRHTGVPDRFTGSGSGTDFTLTISSVQAEDLALYYCQQHYTIPFTFGSGTKLEIK

SEQ ID NO: 333 - protein, 118 aa, synthetic construct
  source 1..118: note = Antibody LK-RB1-26 VH
QVQLKESGPGLVAPSQSLSITCTVSGFSLTSYGVSWVRQPPGKGLEWLGIIWGDGSTNYHSALISRLSISKDNSKSQVFLKLNSLQTDDTATYYCAKPNWDYYAMDYWGQGTSVTVSS

SEQ ID NO: 334 - protein, 107 aa, synthetic construct
  source 1..107: note = Antibody LK-RB1-26 VL
DIQMTQTTSSLSASLGDRVTISCRASQDISNFLNWYQQKPDGTFKLLISYTSTLHSGVPSRFSGSGSGTDYSLTISNLEQEDVATYFCQQGNTLPLTFGAGTKLALK

SEQ ID NO: 335 - protein, 117 aa, synthetic construct
  source 1..117: note = Antibody LK-RB1-27 VH
QVQLQQSGPELVKPGASVRMSCKASGYTFTSHYIHWMKQRPGQGLEWIGWIYPGDGHTKYHEKFKDRATLTADTSSITAYMRLSSLTSEDSAIYYCVYYSDSWFPYWGQGTLVTVSA

SEQ ID NO: 336 - protein, 107 aa, synthetic construct
  source 1..107: note = Antibody LK-RB1-27 VL
DIQMTQSPASLSVSVGETVTITCRASENIYSNLAWYQQKQGKSPHLLVYAATNLADGVPSRFSGSGSGTQYSLKINSLQSEDFGNYYCQHFWGSPWTFGGGTKLEIK

SEQ ID NO: 337 - protein, 117 aa, synthetic construct
  source 1..117: note = Antibody LK-RB1-28 VH
QVQLQQSGPELVKPGTSVKMSCKASGYTFTSHYIHWMKQRPGQGLDWIGWIYPGDGHTKYHEKFKGKATLTADTSSITAYMQLSLLTSEDSAVYYCVYYSDSWFPYWGQGTLVTVSA

SEQ ID NO: 338 - protein, 107 aa, synthetic construct
  source 1..107: note = Antibody LK-RB1-28 VL
DIQMTQSPASLSVSVGETVTITCRASENIYSNLAWYQQKQGKSPQLLVYAATNLADGVPSRFSGSGSGTQYSLKINSLQSEDFGNYFCQHFWGSPWTFGGGTKLEIK

SEQ ID NO: 339 - protein, 118 aa, synthetic construct
  source 1..118: note = Antibody LK-RB1-29 VH
QVQLKESGPGLVAPSQNLAITCTVSGFSLTNSGVSWVRQSPGKGLEWLGIIWGDGNTNYHSALISRLSISKDNSKSQVFLKLNSLQTDDTGTYYCAKPNWDYYALDYWGQGTSVTVSS

SEQ ID NO: 340 - protein, 107 aa, synthetic construct
  source 1..107: note = Antibody LK-RB1-29 VL
DIQMTQTASSLFASLGDRVTISCRASQDISNYLNWYQQKPDGSIKLLISYTSRLHSGVPSRFSGSGSGTDYSLSISNLEQEDIATYFCQQGISLPCTFGGGTKLEVN

SEQ ID NO: 341 - protein, 118 aa, synthetic construct
  source 1..118: note = Antibody LK-RB1-30 VH
QVQLKESGPGLVAPSQSLSITCTVSGFSLTSYGVSWIRQPPGKGLEWLGVIWGDGNTNYHSALISRLSISKDNSKSQVFLKLNSLQTDDTATYYCAKPSWDYYAMDYWGQGTSVTVSS

SEQ ID NO: 342 - protein, 107 aa, synthetic construct
  source 1..107: note = Antibody LK-RB1-30 VL
VIQMTQTTSSLSASLGDRVTISCRASQDISNYVNWYQQRPDGTVKLLISYTSRLHSGVPSRFSGSGSGTDYSLTITNLEQEDIATYFCQQGITLPCTFGGGTKLEIN

SEQ ID NO: 343 - protein, 118 aa, synthetic construct
  source 1..118: note = Antibody LK-RB1-31 VH
QVQLKESGPGLVAPSQSLSITCTVSGFSLTSYGVSWVRQPPGKGLEWLGIIWGDGSTNYHSALKSRLSISKDNSKSQVFLKLNSLQTEDTATYYCAKPNWDYYAMDYWGQGTSVTVSS

SEQ ID NO: 344 - protein, 107 aa, synthetic construct
  source 1..107: note = Antibody LK-RB1-31 VL
DIQMTQTTSSLSASLGDRVTISCRASQDISNYLNWYLQKPDGTVKLLIYYTSRLHSGVPSRFSGSGSGTDYSLTISNLEQEDIATYFCQQGITLPCTFGGGTKLEIK

SEQ ID NO: 345 - protein, 117 aa, synthetic construct
  source 1..117: note = Antibody LK-RB1-32 VH
QVQLQQSGAELAKPGASVKLSCTASGYTFTTYLMHWVKQRPAQGLEWIGYINPSNDFTKYNQKFKDKATLTADTSSSTAYMQLNSLTFEDSAVYYCARNSDYDPSYWGQGTTLTVSS

SEQ ID NO: 346 - protein, 106 aa, synthetic construct
  source 1..106: note = Antibody LK-RB1-32 VL
DIQMTQSPASLSVSVGETVTITCRASENIYSSLAWYQQKQGKSPQLLVYAATNLADGVPSRFSGSGSGTQYSLKINSLQSEDFGNYYCQHFWGTWAFGGGTKLEIK

SEQ ID NO: 347 - protein, 121 aa, synthetic construct
  source 1..121: note = Antibody LK-RB1-33 VH
QIQLVQSGPELKKPGDTVKISCKASGYTFTSYGMNWVKQAPGEGLKWMGWINTYSGVPTYGDDFKGRFAFSLETSASTAYLQINNLENEDTATYFCARRGRDSVVGHFDVWGTGTTVTVSS

SEQ ID NO: 348 - protein, 107 aa, synthetic construct
  source 1..107: note = Antibody LK-RB1-33 VL
DIVMTQSHKFMSTSVGDRVSITCKASQDVNTAVAWYQQKPGQSPKLLIYWASTRHTGVPDRFTGSGSGTDFTLTISSVQAEDLALYYCQQHYTIPFTFGSGTRLEIK

SEQ ID NO: 349 - protein, 123 aa, synthetic construct
  source 1..123: note = Antibody LK-RB1-34 VH
QVQLQQSGAELVKPGASVKLSCKASGYTFTEYTIHWVKQRSGQGLEWIGWFYPGSDSIKYNEKFKDKATLTADKSSSTVYMELSRLTSEDSAVYFCSRHEGPYFTSQYYFDYWGQGTTLTVSS

SEQ ID NO: 350 - protein, 107 aa, synthetic construct
  source 1..107: note = Antibody LK-RB1-34 VL
DILLTQSPAILSVSPGERVSFSCRASRYIGTSIHWYQQRTNGSPRLLIKYASESISGIPSRFSGSGSGTDFTLSINSVESEDIADYYCQQSNTWPLTFGAGTKLELK

SEQ ID NO: 351 - protein, 117 aa, synthetic construct
  source 1..117: note = Antibody LK-RB1-35 VH
DVQLQESGPGLEKPSQSLSLTCSVTGYSITRGYQWNWIRQFPGNKLEWMGYISYDGSNNYNPSLKNRISITRDASKNQFFLKLNSVTTEDTATYYCARRSYNSIDYWGQGTTLTVSS

SEQ ID NO: 352 - protein, 107 aa, synthetic construct
  source 1..107: note = Antibody LK-RB1-35 VL
DIVLTQSPATLSVTPGDSVSLSCRASQSISSNLHWYQQKSHESPRLLIKYASQSISGIPSRFSGSGSGTDFTLSINSVETEDFGMYFCQQSNSWPLTFGAGTKLELK

SEQ ID NO: 353 - protein, 117 aa, synthetic construct
  source 1..117: note = Antibody LK-RB1-36 VH
DVQLQESGPGLEKPSQSLSLTCSVTGYSITRGYQWNWIRQFPGNKLEWMGYISYDGSNNYNPSLKNRISIIRDTSKNQFFLKLNSVTTEDTATYYCARRSYNSIDYWGQGTTLTVSS

SEQ ID NO: 354 - protein, 107 aa, synthetic construct
  source 1..107: note = Antibody LK-RB1-36 VL
DIVLTQSPATLSVTPGDSVSLSCRASQSISNNLHWYQQKSHESPRLLIKYASQSISGIPSRFSGSGSGTDFTLSINSVETEDFGMYFCQQSNSWPLTFGAGTKLELK

SEQ ID NO: 355 - protein, 121 aa, synthetic construct
  source 1..121: note = Antibody LK-RB1-37 VH
QVQLQQPGAELVKPGASVKLSCKASGYTFTSYWMHWVKQRPGRGPEWIGRIDPNSGYARYNEKFKNKATLTADKPSSTADMHLSSLTSEDSAVYYCTRSFITTAEWFFDVWGTGTTVTVSS

SEQ ID NO: 356 - protein, 107 aa, synthetic construct
  source 1..107: note = Antibody LK-RB1-37 VL
DILLTQSPAILSVSPGERVSFSCRASQSIGTSIHWYQQRRNGSPRLLIKYTSESISGIPSRFSGSGSGTDFTLSINSVESEDIADYYCQQSNSWPHTFGGGTKLELK

SEQ ID NO: 357 - protein, 117 aa, synthetic construct
  source 1..117: note = Antibody LK-RB1-38 VH
DVQLQESGPGLVQPSQSLSLTCSVTGYSITSGYQWNWIRQLPGNKLEWMAYISFDGSNNYNPSLKNRISITRDTSKNQFFLKLKSVTTEDTATYYCARRDYDGVAYWGQGTLVTVSA

SEQ ID NO: 358 - protein, 107 aa, synthetic construct
  source 1..107: note = Antibody LK-RB1-38 VL
EIVLTQSPATLSVTPGDRVSLSCRASQNISNYLHWYQQKSHGSPRLIIKYASQSISGIPSRFSGSGSGTDFTLSINSVETEDFGMYFCQQSNSWPLTFGAGTKLELK

SEQ ID NO: 359 - protein, 116 aa, synthetic construct
  source 1..116: note = Antibody LK-RB1-39 VH
EVQLQQSGAELVRPGASVKLSCTASGFNIKDDYVHWVNQRPEQGLEWIGWIDPENGDTGYASKFQDKATITADTSSNTAYLHLSSLTSEDTAVYYCTTTRWTGSYWGQGTTLTVSS

SEQ ID NO: 360 - protein, 108 aa, synthetic construct
  source 1..108: note = Antibody LK-RB1-39 VL
QIVLTQSPAIMSASPGEKVTITCSASSSVNYMHWFQQKPGSSPKLWIYSTANLASGVPARFSGSGSGTSYSLTISRMEAEDAATYYCQQRSSYPPTWTFGGGTKLEIK

SEQ ID NO: 361 - protein, 117 aa, synthetic construct; identical to SEQ ID NO: 357
  source 1..117: note = Antibody LK-RB1-40 VH

SEQ ID NO: 362 - protein, 112 aa, synthetic construct
  source 1..112: note = Antibody LK-RB1-40 VL
DVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPWTFGGGTKLEIK

SEQ ID NO: 363 - protein, 117 aa, synthetic construct; identical to SEQ ID NO: 357
  source 1..117: note = Antibody LK-RB1-41 VH

SEQ ID NO: 364 - protein, 107 aa, synthetic construct; identical to SEQ ID NO: 358
  source 1..107: note = Antibody LK-RB1-41 VL

SEQ ID NO: 365 - protein, 121 aa, synthetic construct
  source 1..121: note = Antibody LK-RB1-42 VH
QVQLQQPGAELVKPGTSVKLSCKASGYSFISYWVYWVKQRPGQGLEWIGMIHPNSGSTNFNEKFRYKATLTVDKSSSTAYIQLSSLTSEDSAVYYCGRSDDGYPYHAMDSWGQGTSVTVSS

SEQ ID NO: 366 - protein, 107 aa, synthetic construct
  source 1..107: note = Antibody LK-RB1-42 VL
DIVMTQSQKFMSTSVGDRVSVTCKASQNVGTNVAWYQQKPGQSPKALIYSASYRFSGVPDRFTGSGSGTDFTLTIGNVQSEDLAEYFCHQYKNYPLTFGGGTKLEIK

SEQ ID NO: 367 - protein, 118 aa, synthetic construct
  source 1..118: note = Antibody LK-RB1-43 VH
EFQLQQSGPELMKPGASVKISCKASGYSFTDYNMNWVKQSNGKSLEWIGIIDPNYGTTTYNQKFKGRATLTVDQSSNTAYMQLNSLTSEDSAVYYCAREGRYSWFAYWGQGTLVTVSA

SEQ ID NO: 368 - protein, 107 aa, synthetic construct
  source 1..107: note = Antibody LK-RB1-43 VL
DIQMTQSPGALSVSVGETVTITCRASENINSNLAWYQQKQGKSPQLLVYAATNLADGVPSRFSGSGSGTQYSLKINSLQSEDFGSYHCQHFWGTPFTFGSGTKLEIK

SEQ ID NO: 369 - protein, 121 aa, synthetic construct
  source 1..121: note = Antibody LK-RB1-44 VH
QVQLQQSGTELAKPGASVKLSCKASGYTFTNYWMHWIKQRPGQGLEWIGYVHPTSDYTKYNQKFKDKATLTADKSSSTAYMQLNSLTYEDSAVYYCTRSSLYDTNYVFDSWGQGTTLTVSS

SEQ ID NO: 370 - protein, 107 aa, synthetic construct
  source 1..107: note = Antibody LK-RB1-44 VL
DILLTQSPAILSVSPGERVSFSCRASQSIGRSIHWFQQRTNGSPSLLIKYASESISGIPSRFSGGGSGTDFTLSISNVESEDIADYYCQQSNNWPFTFGSGTKLEIK

SEQ ID NO: 371 - protein, 121 aa, synthetic construct
  source 1..121: note = Antibody LK-RB1-45 VH
QVQLQQSGTELARPGASVKLSCKASGYTFTNFWMHWIKQRPGQGLEWIGYVNPTSDYTKYNQKFKDKATLTADKSSSTAYMQLNSLTYEDSAVYYCTRSSLYDTNYVFDSWGQGTTLTVSS

SEQ ID NO: 372 - protein, 107 aa, synthetic construct
  source 1..107: note = Antibody LK-RB1-45 VL
DILLTQSPAILSVSPGERVSFSCRASQSIGRSIHWYQQRTNGSPRLLIKYASESISGIPSRFSGGGSGTDFTLSISNVESEDIADYYCQQSNNWPFTFGSGTKLEIK

SEQ ID NO: 373 - protein, 120 aa, synthetic construct
  source 1..120: note = Antibody LK-RB1-46 VH
EVQLQQSGPELVKPGASVKISCKTSGYTFTDYYMNWVKQSHGKSLEWIGDIYPINGDISYNQKFKGKATLTVDKSSSTVYMEIRSLTYEDSAVYYCARLAQTTSGGFDYWGQGTTLTVSS

SEQ ID NO: 374 - protein, 112 aa, synthetic construct
  source 1..112: note = Antibody LK-RB1-46 VL
DIVLTQSPTSLAVSLGQRAIISCKASQSVSFAGTSLMHWYQQKPGQQPKLLIYRASNLKAGVPTRFSGSGSRTDFTLNIHPVEEDDAATYYCQQSREYPMYTFGGGTKLEIK

SEQ ID NO: 375 - protein, 122 aa, synthetic construct
  source 1..122: note = Antibody LK-RB1-47 VH
QIQLVQSGPELKKPGETVKISCKASGYTFTTYGMSWVKQAPGKGLKWMGWINTYSGVPTYTDDFKGRFAFSVETSASTAYLQINNLKNEDTATYFCARRERDFYGNIFFPYWGQGSLVTVSA

SEQ ID NO: 376 - protein, 107 aa, synthetic construct
  source 1..107: note = Antibody LK-RB1-47 VL
DIQMTQTTSSLSASLGDRVTISCRASQDISNYLNWYQQKPDGTVKLLIYYTSRIHSGVPSRFSGSGSGTDFSLTISNLDQEDIATYFCQQGHTLPWTFGGGTKVEIK

SEQ ID NO: 377 - protein, 119 aa, synthetic construct
  source 1..119: note = Antibody LK-RB1-48 VH
EVKLVESGGGLVQPGGSLKLSCAASGFTFSDYYMYWVRQTPEKRLEWVAYISNGGGISFYPDTVKGRFTISRDNAKNTLYLQMSRLKSEDTAIYYCASATKEVGAMDYWGQGTSVTVSS

SEQ ID NO: 378 - protein, 106 aa, synthetic construct
  source 1..106: note = Antibody LK-RB1-48 VL
DIVMTQSQKFLSTTVGDRVSITCKASQNVGTAVAWYQQKPGQSPKILIYSASNRYTGVPDRFTGSGSGTDFTLTISNMQSEDLADYFCQQYSSYRTFGGGTKLEIK

SEQ ID NO: 379 - protein, 121 aa, synthetic construct
  source 1..121: note = Antibody LK-RB1-49 VH
QIQLVQSGPELKKPGETVKISCKASGYTFTTYGMSWVKQAPGKGLKWMGWINTYSGVPTYADDFKGRFAFSLETSGSTAYLQISNLKNEDTATYFCSRLFRSWDSYAMDYWGQGTSVTVSS

SEQ ID NO: 380 - protein, 107 aa, synthetic construct
  source 1..107: note = Antibody LK-RB1-49 VL
DIVMTQSQKFMSATVGDRVSITCKASQNVGTAVAWYQQKPGQSPKLLIYSPSNRYTGVPDRFTGSGSGTHFTLTINNMQSEDLADYFCQQYSTYPLTFGAGTKLELN

SEQ ID NO: 381 - protein, 121 aa, synthetic construct
  source 1..121: note = Antibody LK-RB1-50 VH
QVQLKQSGPGLVQPSQSLSITCTVSGFSLTSYGVHWVRQSPGKGLEWLGVIWRGGNTDYNAPFVSRLSITKDNSQSQVFFKMNSLEADDTAIYYCAKNSLDVYSSWYFDVWGTGTTVTVSS

SEQ ID NO: 382 - protein, 106 aa, synthetic construct
  source 1..106: note = Antibody LK-RB1-50 VL
QIVLSQSPAILSAFPGEKVTMTCRASSSVSFMHWYQQKPGSSPKPWIYATSNLASGVPARFSGSGSGTSYSLTISRVEAEDAATYYCQQWSSNPLTFGAGTKLELK

SEQ ID NO: 383 - protein, 120 aa, synthetic construct
  source 1..120: note = Antibody LK-RB1-51 VH
QVQIQQSGAELVKPGASVKLSCKASGYTFTEFPIHWIKQRSGQGLEWIGWFYPGSDNMNYNEKFKDKATLTADKSSSTVYMELSRLTSEDSAVYFCARHENAYFYAMDYWGQGTSVTVSS

SEQ ID NO: 384 - protein, 106 aa, synthetic construct
  source 1..106: note = Antibody LK-RB1-51 VL
ENVLTQSPAIMSASLGEKVTMSCRASSSVNYMYWYQQKSDASPKIWIYYTSNLAPGVPARFSGSGSGNSYSLTISSMEGEDAATYYCQQFTSSPFTFGTGTKLEIK

SEQ ID NO: 385 - protein, 119 aa, synthetic construct
  source 1..119: note = Antibody LK-RB1-52 VH
EVKLVESGGGLVQPGGSLKLSCAASGFIFSDYYMYWVRQTPEKRLEWVAYISNGGDITYYPDTVKGRFTISRDIARNTLFLQMGRLKSEDTAMYYCASATTEVGAMDHWGQGTSVTVSS

SEQ ID NO: 386 - protein, 106 aa, synthetic construct
  source 1..106: note = Antibody LK-RB1-52 VL
DIVMTQSQKFMSTTVGDRVSITCKASQNVGTAVAWYQQKPGQSPKLLIYAASNRYTGVPDRFTGSGSGTDFTLTISNMQSEDLADYFCQQYRSYRTFGGGTNLEIK

SEQ ID NO: 387 - protein, 123 aa, synthetic construct
  source 1..123: note = Antibody LK-RB1-53 VH
QIQLVQSGPELKKPGETVKISCKASGYTFTTYGMSWVNQAPGKGLKWMGWINTNYGVPTYADDFKGRFAFSLETSASTAYLQINNLKNEDTATYFCARGTRRRGISYFAMDYWGQGTSVTVSS

SEQ ID NO: 388 - protein, 107 aa, synthetic construct
  source 1..107: note = Antibody LK-RB1-53 VL
DIVMTQSQKFMSTTVGDRVSITCKASQNVGTAVAWYQQKPGQSPKLLIYSASNRYTGVPDRFTGSDSGTDFTLTINNIQSEDLADYFCQQYSSYPLTFATGTKLELK

SEQ ID NO: 389 - protein, 120 aa, synthetic construct
  source 1..120: note = Antibody LK-RB1-54 VH
DVQLQESGPGLVKPSQSLSLTCSVTGYSITSGFYWNWIRQFPGNKLEWMGHISYDGTNNYNPSLKNRISITRDTSKNLFFLKLNSVTTEDTATFHCARGGYGGSYGFDYWGQGTTLTVSS

SEQ ID NO: 390 - protein, 106 aa, synthetic construct
  source 1..106: note = Antibody LK-RB1-54 VL
ENVLTQSPAIMSASLGEKVTMSCRASSSVNYMYWYQQKSDASPKLWIYYTSNLAPGVPVRFSGSGSGNSYSLTISSMEGEDAATYYCQQFTSSPWTFGGGTKLEIK

SEQ ID NO: 391 - protein, 119 aa, synthetic construct
  source 1..119: note = Antibody LK-RB1-55 VH
EVKLVESGGGLVQPGGSLKLSCAASGFTFSDYYMYWVRQTPEKRLEWVAYISNGGDITFYPDTVKGRFTISRDNARSTLYLQMSRLKSEDTAIYYCASATTEVGAMDHWGQGTSVTVSS

SEQ ID NO: 392 - protein, 106 aa, synthetic construct
  source 1..106: note = Antibody LK-RB1-55 VL
DIVMTQSQKFMSTTVGDRVSITCKASQNVGTAVAWYQQKPGKSPKLLIYAASNRYTGVPDRFTGSGSGTDFTLTISNMQSEDLADYFCQQYSSYRTFGGGTNLEIK

SEQ ID NO: 393 - protein, 121 aa, synthetic construct
  source 1..121: note = Antibody LK-RB1-56 VH
QVQLKQSGPGLVQPSQSLSITCSVSGFSLTNYGVHWVRQSPGKGLEWLGVIWRGGSTDNNAAFMSRLSITKDNSKSQVFFKMNSLQADDTAIYYCAKNSDFYGSSWYFDVWGTGTTVTVSS

SEQ ID NO: 394 - protein, 106 aa, synthetic construct
  source 1..106: note = Antibody LK-RB1-56 VL
QIVLSQSPAILSASPGEKVTMTCRASSSVSFMHWYQQKPGSSPKPWIYATSNLASGVPARFSGSGSGTSYSLTISRVEAEDAATYYCQQWSRNPPTFGAGTKLELK

SEQ ID NO: 395 - protein, 119 aa, synthetic construct
  source 1..119: note = Antibody LK-RB1-57 VH
EVKLVESGGGLVQPGGSLKLSCAASGFTFSDYYMYWVRQTPEKRLEWVAYISNGGDITFYPDTVKGRFTISRDNARSTLYLQTSRLKSEDTAIYYCASATTEVGAMDHWGQGTSVTVSS

SEQ ID NO: 396 - protein, 106 aa, synthetic construct
  source 1..106: note = Antibody LK-RB1-57 VL
DIVMTQSQKFMSTTVGDRVSITCKASQNVGTAVAWYQQKPGKSPKLLIYAASNRYTGVPDRFTGSGSGTDFTLTISNVQSEDLADYFCQQYSSYRTFGGGTSLEIK

SEQ ID NO: 397 - protein, 121 aa, synthetic construct
  source 1..121: note = Antibody LK-RB1-58 VH
QVQLKQSGPGLVQPSQSLSITCTVSGFSLTTYGVHWVRQSPGKGLEWLGVIWRGGSTDYNAAFMSRLSITKDNSKSQVFFRMNSLQADDTARYYCAKNSLYYGSSWYFDVWGTGTTVTVSS

SEQ ID NO: 398 - protein, 106 aa, synthetic construct
  source 1..106: note = Antibody LK-RB1-58 VL
QIVLSQSPAILSASPGEKVTMTCRASSSVSFMHWYQQKPGSSPKPWIYATSNLASGVPARFSGSGSGTSYSLTISRVEAEDAATYYCQQWSSNPPTFGGGTKLEIK

SEQ ID NO: 399 - protein, 123 aa, synthetic construct
  source 1..123: note = Antibody LK-RB1-59 VH
QIQLVQSGPELKKPGETVKISCKASGYTFTTYGMSWVKQAPGKGLKWMGWINTYSGVPTYADDFKGRFAFSLETSASTAYLQINNLKNEDTATYFCARRRIYYGNKGYAMDYWGQGTSVTVSS

SEQ ID NO: 400 - protein, 107 aa, synthetic construct
  source 1..107: note = Antibody LK-RB1-59 VL
DIQMTQSPASLSVSVGETVTITCRASENIYSNLVWYQQKQGKSPQLLVYTATNLADGVPSRFSGSGSGTQFSLKINSLQSEDFGSYYCQHFWGTPWTFGGGTKLEIK

SEQ ID NO: 401 - protein, 120 aa, synthetic construct
  source 1..120: note = Antibody LK-RB2-1 VH
QIQLVQSGPELKKPGETVKISCKASGYTFTTYGMSWVKQAPGKGLKWMGWINTYSGVSTYADDFKGRFAFSLETSASTAYLQINNLKNEDTATYFCARRYDYDGYAMDYWGQGTSVTVSS

SEQ ID NO: 402 - protein, 113 aa, synthetic construct
  source 1..113: note = Antibody LK-RB2-1 VL
DIVMSQSPSSLAVSVGEKVTMSCKSSQSLLFSSNQKNYLAWYQQKPGQSPKLLIYWASTRESGVPDRFTGRGSGTDFTLTISSVKTEDLAVYYCQQYDTYPPTFGGGTKLEIK

SEQ ID NO: 403 - protein, 124 aa, synthetic construct
  source 1..124: note = Antibody LK-RB2-2 VH
DVKLEESGEGLVKPGGSLKLSCAASGFTFSSYAMSWVRQTPEKRLEWVAYISSGGDYFHYADTVKGRFTISRDNARNTLYLQMSSLKSEDTAMYYCTREGAHYDSSYLAWFAHWGQGTLVTVSA

SEQ ID NO: 404 - protein, 107 aa, synthetic construct
  source 1..107: note = Antibody LK-RB2-2 VL
DIQMTQSPASLSVSVGETVTITCRASENIYSNLAWYQQRQGKSPQLLVYAATNLADGVPSRFSGSGSGTQYSLKINSLQSEDFGSYYCQQFWDNPLTFGAGTKLELK

SEQ ID NO: 405 - protein, 114 aa, synthetic construct
  source 1..114: note = Antibody LK-RB2-3 VH
QLQLQQSDAELVKPGASVKISCKVSGYSFTDHTIHWMKQRPEQGLEWIGYIYPRDGSTKENEKFTGKATLTADKSSSTAYMQLTSLTSEDSAVYFCTGSGFDYWGQGTSLTVSS

SEQ ID NO: 406 - protein, 107 aa, synthetic construct
  source 1..107: note = Antibody LK-RB2-3 VL
DIKMTQSPSSMYASLGERVTITCKASQDIKSYLSWFQQKPGKSPKTLIYRANRLVDGVPSRFSGSGSGQDYSLTINSLEYEDMEIYYCLQYDEFPYTFGGGTKLEIK

SEQ ID NO: 407 - protein, 123 aa, synthetic construct
  source 1..123: note = Antibody LK-RB2-4 VH
EVQLVESGGDLVKPGGSLKLSCAASGFTFSNYGMSWVRQTPDKRLEWVATISSGGSYTFYPDSVKGRFTISRDNAKNTLYLQMSSLKSEDTALYYCARDYYYGKTIEGYFDYWGQGTTLTVSS

SEQ ID NO: 408 - protein, 107 aa, synthetic construct
  source 1..107: note = Antibody LK-RB2-4 VL
DIQMTQTTSSLSASLGDRVTISCRANQDISNYLNWYQQKPDGTVKLLIYYTSRLHSGVPSRFSGSGSGTDYSLTISDLEQEDIATYFCQQGNTLPWTFGGGTKLEIK

SEQ ID NO: 409 - protein, 121 aa, synthetic construct
  source 1..121: note = Antibody LK-RB2-5 VH
QIQLVQSGPELKKPGETVKISCKASGYTFTTYGMSWVKQAPGKGLKWMGWINTYSGVPTYADDFKGRFAFSLETSVSTAYLQINNLKNEDTATYFCARRDGYDGGGNFDYWGQGTTLTVSS

SEQ ID NO: 410 - protein, 107 aa, synthetic construct
  source 1..107: note = Antibody LK-RB2-5 VL
DIVMTQSPATLSVTPGDRVSLSCRASQSISDYLHWYQQKSHESPRLLIKYASQSISGIPSRFSGSGSGSDFTLSINSVEPEDVGVYYCQNGHGFPWTFGGGTKLEIK

SEQ ID NO: 411 - protein, 120 aa, synthetic construct
  source 1..120: note = Antibody LK-RB2-6 VH
QIQLVQSGPELKKPGETVKISCKASGYTFTTYGMSWVKQAPGKGLEWMGWINIYSGVPTYADDFKGRFAFSLETSASTAYLQINNLKNEDTATYFCARRGDYDSFYFDYWGQGTTLTVSS

SEQ ID NO: 412 - protein, 113 aa, synthetic construct
  source 1..113: note = Antibody LK-RB2-6 VL
DIVMSQSPSSLAVSVGEKVTVSCKSSQSLLYSYNQKNYLAWYQQKPGQSPKLLIYWSSTRESGVPDRFTGSGSGTDFTLTISSVKAEDLAVYYCQQCYNYPYTFGGGTKLEIK

SEQ ID NO: 413 - protein, 124 aa, synthetic construct
  source 1..124: note = Antibody LK-RB2-7 VH
DVKLVESGEGLVKPGGSLKLSCAASGFTFSSYAMSWVRQTPEMRLEWVAYISSGGDHFYYADTVKGRFTISRDNARSTLYLQMSSLKSEDTAMYYCTREGASYDSNFLAWFAYWGQGTLVTVSA

SEQ ID NO: 414 - protein, 107 aa, synthetic construct
  source 1..107: note = Antibody LK-RB2-7 VL
DIQMTQSPASLSVSVGETVTITCRASENIYSNLAWYQQKEGKSPQLLVYGATNLADGVPSRFSGSGSGTQYSLKINSLQSEDFGSYYCQNFWDIPLTFGAGTKLELK

SEQ ID NO: 415 - protein, 117 aa, synthetic construct
  source 1..117: note = Antibody LK-RB2-8 VH
QIQLVQSGPELKKPGETVKISCKASGYSFASYGMGWVKQAPGKGLKWLGWINTHSGVPTYADDFKGRFAFSLETSASTAYLQINNLKNEDTATYFCAHYGRPWFPYWGQGTLVTVSA

SEQ ID NO: 416 - protein, 107 aa, synthetic construct
  source 1..107: note = Antibody LK-RB2-8 VL
DIQMTQSPASLSASVGETVTITCRTSENIYSYLAWYQQKQGKSPQLLVYNAKSLAEGVPSRFSGSGSGTQFSLRINSLQPEDFGTYYCQQHYETPLTFGAGTKLELK

SEQ ID NO: 417 - protein, 121 aa, synthetic construct
  source 1..121: note = Antibody LK-RB2-9 VH
DVQLQESGPGLVKPSQSLSLTCSVTGYSITSGYYWNWIRQFPGNKLEWMGYIGYDGNNNYNPSLKNRISITRDTSKNQFFLKLNSLTTEDTATYYCAREGSTMVTSWFAYWGQGTLVTVSA

SEQ ID NO: 418 - protein, 107 aa, synthetic construct
  source 1..107: note = Antibody LK-RB2-9 VL
DIQMTQSPASLSVSVGETVTITCRASENIYSNLAWYQQKQGKSPQLLVYGATNLADGVPSRFSGRGSDTQYSLKINSLQSEDFGSYYCQHFWDTPYTFGGGTKLEIK

SEQ ID NO: 419 - protein, 121 aa, synthetic construct
  source 1..121: note = Antibody LK-RB2-10 VH
EVMLVESGGDLVKPGGSLKLSCAASGFTFSANTVSWLRRTPERRLEWVATISGGSDNTYCPDSVKGRFTISRDNARDTLYLQMSSLTSEDTALYFCARHYYDGPNWYFDVWGPGTTVTVSS

SEQ ID NO: 420 - protein, 107 aa, synthetic construct
  source 1..107: note = Antibody LK-RB2-10 VL
DIVMTQSQKFMSSTGGDRVTITCKASQNVDTAVAWYQQKPGQSPKLLIYSASNRYIGVPDRFKGSGSGTDFSLTITNIQSEDLAEYFCQQYSSYPLTFGAGTKLELK

SEQ ID NO: 421 - protein, 118 aa, synthetic construct
  source 1..118: note = Antibody LK-RB2-11 VH
QIQLVQSGPELKKPGETVKISCKASGYSFITYGMSWVKQAPGKGLKWMGWINTYSGEPTYGDDFMGRFAFSLETSASTAYLQINNLKNEDTASYFCARYHYDDGMDYWGQGTSVTVSS

SEQ ID NO: 422 - protein, 107 aa, synthetic construct
  source 1..107: note = Antibody LK-RB2-11 VL
DIKMTQSPSSMYASLGERVTITCKASQDINNYLSWFQQKPGKSPKTLIFRANRLVDGVPSRFSGSGSGQDYSLTISSLEDEDMGIYYCLQYDEFPWMFGGGTKLEIE

SEQ ID NO: 423 - protein, 120 aa, synthetic construct
  source 1..120: note = Antibody LK-RB2-12 VH
QIQLVQSGPELKKPGETVKISCKASGYTFTTYGMSWVKQTPGKGLKWMGWINIYSGVPTYADDFKGRFVFSLEISASTAHLQINNLKNEDTATYFCARRKDYSGHCFDYWGQGTTLTVSS

SEQ ID NO: 424 - protein, 111 aa, synthetic construct
  source 1..111: note = Antibody LK-RB2-12 VL
DIVLTQSPASLAVSPGQRATISCRASESVDTYGISFMHWYQQKPGQPPKLLIYRASNLESGIPARFSGSGSRTDFTLTINPVETDDVAIYYCQQSKKDPYTFGGGTKLEIK

SEQ ID NO: 425 - protein, 120 aa, synthetic construct
  source 1..120: note = Antibody LK-RB2-13 VH
QIQLVQSGPELKKPGETVKISCKASGYTFTTYGMSWVKQAPGKGLQWMGWINTYSGVPTFADDFKGRFAFSLDTSASTAYLQINNLKNEDTATYFCARRINWDGYAMDYWGQGTSVTVSS

SEQ ID NO: 426 - protein, 113 aa, synthetic construct
  source 1..113: note = Antibody LK-RB2-13 VL
DSVMSQSPSSLAVSVGEKVTMSCKSSQSLLYSGNQKSYLAWYQQKPGQSPKLLIYWASTRESGVPDRFTGSGSGTDFTLTISSVKAEDLAVYYCQQYYSYPYTFGGGTKLEIK

SEQ ID NO: 427 - protein, 122 aa, synthetic construct
  source 1..122: note = Antibody LK-RB2-14 VH
QVQLKQSGPGLVQPSQSLSITCTVSGFSLTSYGVHWVRQSPGKGLEWLGVIWRGGSTDYNAAFMSRLSITKDNSKSQVFFKMNSLQADDTAIYYCAKNEGDSTVVAWYFDVWGTGTTVTVSS

SEQ ID NO: 428 - protein, 107 aa, synthetic construct
  source 1..107: note = Antibody LK-RB2-14 VL
DIQMTQTTSSLSASLGDRVTISCSASQGISNYLNWFQQKPDGTVKLLIYYTSSLHSGVPSRFSGSGSGTDYSLTISNLEPEDIATYYCQQYSKLPLTFGAGTKLELK

SEQ ID NO: 429 - protein, 120 aa, synthetic construct
  source 1..120: note = Antibody LK-RB2-15 VH
QIQLVQSGPELKKPGETVKISCKASGYTFTTYGMSWVKQAPGKGLKWMGWINVYSGVPTYADDFKGRFAFSLEISASTAYLQINNLKDEDTATYFCARRKDYSGHCFDYWGQGTTLTVSS

SEQ ID NO: 430 - protein, 111 aa, synthetic construct
  source 1..111: note = Antibody LK-RB2-15 VL
DIVLTQSPASLAVSLGQRATFSCRASESVDTYGISFMHWYQQKPGQPPKLLIYRASNLESGIPARFSGSGSRTDFTLTINPVETDDVATYYCQQSKKEPYTFGGGTKLEIK

SEQ ID NO: 431 - protein, 117 aa, synthetic construct
  source 1..117: note = Antibody LK-RB2-16 VH
QIQLVQSGPELKKPGETVKISCKASGYTFTTYGMGWVKQAPEKGLKWMGWINTNSGAPTYGDDFKGRFAFSLETSASTAYLQINNLKNEDTATYFCAHYGRPWFAYWGQGTLVTVSA

SEQ ID NO: 432 - protein, 107 aa, synthetic construct
  source 1..107: note = Antibody LK-RB2-16 VL
DIQMTQSPASLSASVGETVTITCRASENIYSYLAWYHQKQGESPQLLVYNAKTLAEGVPSRFSGSGSGTQFSLKINSLQPEDFGSYYCQHHYNIPLTFGAGTKLELK

SEQ ID NO: 433 - protein, 120 aa, synthetic construct
  source 1..120: note = Antibody LK-RB2-17 VH
QIQLVQSGPELKKPGETVKISCKASGYTFTNYGMSWVKQAPGKGLQWMGWINTYSGVPTFADDFRGRFAFSLDTSASTAYLQINSLKNEDTAAYFCARRINWDGYAMDYWGQGTSVTVSS

SEQ ID NO: 434 - protein, 113 aa, synthetic construct
  source 1..113: note = Antibody LK-RB2-17 VL
DSVMSQSPSSLAVSVGEKVTLSCKSSQSLLYSGNQKSYLAWYQQKPGQSPKLLIYWASSRESGVPDRFTGSGSGTDFTLTISSVKAEDLAVYYCQQYYSFPYTFGGGTKLEIK

SEQ ID NO: 435 - protein, 123 aa, synthetic construct
  source 1..123: note = Antibody LK-RB2-18 VH
EVQLQQSGPELVKPGTSVKISCKASGYTFTDYYMNWVKQSHGKSLEWIGDINPNNGGTSYNQKFKGKATLTVDKSSSTAYMELRSLTSEDSAVFYCARSGTTVVGTYWYFDVWGTGTTVTVSS

SEQ ID NO: 436 - protein, 107 aa, synthetic construct
  source 1..107: note = Antibody LK-RB2-18 VL
DIQMTQSPASLSVSVGETVTITCRASENIYSNLAWYQQKQGKSPQLLVYGATNLADGVPSRFSGSGSGTQYSLKINSLQSEDFGSYYCQHFWDTPFTFGSGTKLEIK

SEQ ID NO: 437 - protein, 121 aa, synthetic construct
  source 1..121: note = Antibody LK-RB2-19 VH
QVQLQQPGAELVKPGASVKLSCKASGYTFTSYWMHWVKQRPGQGLEWIGMIHPNSGGTNYNEKFKSKATLTVDKSSSTAYMQFSSLTSEDSAVYYCARCTVVALYYALDYWGQGTSVTVSS

SEQ ID NO: 438 - protein, 106 aa, synthetic construct
  source 1..106: note = Antibody LK-RB2-19 VL
QIVLSQSPAILSASPGEKVTMTCRATSRVSYIHWFQQKPGSSPKPWIYVTSNLASGVPARFSGSGSGTSYSLTISKLEAEDVATYYCQQWSTNPLTFGAGTKLELK

SEQ ID NO: 439 - protein, 122 aa, synthetic construct
  source 1..122: note = Antibody LK-RB2-20 VH
QVQLKQPGAERVKPGASVKMSCKASGYTFTRYWITWVKQRPGQGLEWIGDIYPGSATTNYNEKFKSKATLTEDTSSSTAYMQLSSLTSEDSAVYYCARSGDYSNSNYAMDYWGQGTSVTVSS

SEQ ID NO: 440 - protein, 107 aa, synthetic construct
  source 1..107: note = Antibody LK-RB2-20 VL
NIEMTQSPKSMSMAVGERVTLSCKASENVGTYVSWYQQKPEQSPKLLIYGASNRNTGVPDRFTGSGSATDFTLTISSVQAEDLADYHCGQSYSYPFTFGSGTKLEIK

SEQ ID NO: 441 - protein, 121 aa, synthetic construct
  source 1..121: note = Antibody LK-RB2-21 VH
EVQLVESGGDLVKPGGSLKLSCAASGFTFSSYGMSWVRQTPDKRLEWVATISSGGSYTYYPDSLKGRFTISRDNAKSTLYLQMSSLKSEDTAKYYCARPYYDYIFYAIDYWGQGTSVTVSS

SEQ ID NO: 442 - protein, 107 aa, synthetic construct
  source 1..107: note = Antibody LK-RB2-21 VL
DIQMNQSPSSLSASLGDTITITCHASQNINVWLSWYQQKPGNIPKLLIYKASNLHTGVPSRFSGSGSGTGFTLTISSLQPEDIATYYCQQGQSYPLTFGGGTKLEIK

SEQ ID NO: 443 - protein, 120 aa, synthetic construct
  source 1..120: note = Antibody LK-RB2-22 VH
QIQLVQSGPELKKTGETVKISCKASGYTFTTYGMSWVKQAPGKGLKWMGWINIYSGVPTYADDFKGRFAFSLETSASTAYLQINNLKNEDTATYFCARRADYDGYYFDYWGQGTTLTVSS

SEQ ID NO: 444 - protein, 107 aa, synthetic construct
  source 1..107: note = Antibody LK-RB2-22 VL
NIVMTQTPKFLLVSAGDRVTITCRASQSVTNDVAWYQQRPGQSLKLLIYYASNRYTGVPDRFTGSGYGTDFTFTISTVQAEDLAVYFCQQGYSSPFTFGSGTKLEIK

SEQ ID NO: 445 - protein, 120 aa, synthetic construct
  source 1..120: note = Antibody LK-RB2-23 VH
QIQLVQSGPELKKPGETVKISCKASGYTFTTYGMSWVKQAPGKGLKWMGWINIYSGVPTYADDFKGRFAFSLETSASTAYLQINNLKNEDTATYFCARRGDYDGYYFDYWGQGTTLTVSS

SEQ ID NO: 446 - protein, 107 aa, synthetic construct
  source 1..107: note = Antibody LK-RB2-23 VL
NIVLIQSAATLSVTPGDSVSLSCRASQSLSNGLHWYQQKSHESPRLLIKYGSQSISGIPSRFSGSGSGTDFTLSINSVETEDFGMYFCQQSDSWPHTFGSGTKLEIK

SEQ ID NO: 447 - protein, 120 aa, synthetic construct
  source 1..120: note = Antibody LK-RB2-24 VH
QIQLVQSGPEVKKPGETVKISCKASGYTFTTYGMSWVKQAPGKGLKWMGWLNIYSGVPAYADDFKGRFAFSLESSASTAYLQINNLKNEDTATYFCARRENYDGYGMDYWGQGTSVTVSS

SEQ ID NO: 448 - protein, 107 aa, synthetic construct
  source 1..107: note = Antibody LK-RB2-24 VL
DIVMTQSPATLSVTPGDRVSLSCRASQSISDYLHWYQQKSHESPRLLIKYASQSISGIPSRFSGSGSGSDFTLSINSVEPEDVGVYYCQNGHSFPYTFGGGTKLEIK

SEQ ID NO: 449 - protein, 118 aa, synthetic construct
  source 1..118: note = Antibody LK-RB2-25 VH
QVQLKESGPGLVAPSQSLSITCTVSGFSLTSYGVHWVRQPPGKGLEWLGVIWAGGSTKYNSALMSRLSINKDNSKSQVFLKMNSLQTDDTAMYYCARDQMVRSAMDYWGQGTSVTVSS

SEQ ID NO: 450 - protein, 107 aa, synthetic construct
  source 1..107: note = Antibody LK-RB2-25 VL
DIQMIQSPSSMFASLGDRVSLSCRASQGIRGNLDWYQQKPGGTIKLLIYSTSNLNSAVPSRFSGSGSGSDYSLTISNLESEDFADYYCLQRNASPYTFGGGTKLEIK

SEQ ID NO: 451 - protein, 118 aa, synthetic construct
  source 1..118: note = Antibody LK-RB2-26 VH
QVQLKESGPGLVAPSQSLSITCTVSGFSLTSYGVHWVRQPPGKGLEWLGVIWAGGSTDYNAAFISRLNISKDISESQVFFRMNSLQTDDTAMYYCARDQMVRSAMDYWGQGTSVTVSS

SEQ ID NO: 452 - protein, 107 aa, synthetic construct; identical to SEQ ID NO: 450
  source 1..107: note = Antibody LK-RB2-26 VL

SEQ ID NO: 453 - protein, 114 aa, synthetic construct
  source 1..114: note = Antibody LK-RB2-27 VH
DVKLVESGGGLVKPGGSLKLSCAASGFTFSSYSMSWVRQTPEKRLEWVATISTGGSYTYYPDSVKGRFTISRDNAKNTLYLQMSSLKSEDTAMFYCTRGSMDYWGQGTSVTVSS

SEQ ID NO: 454 - protein, 107 aa, synthetic construct
  source 1..107: note = Antibody LK-RB2-27 VL
DILMTQSPSSMSVSLGDTVSITCHASQDISSNIGWLQQKPGNSFKGLIYHGTNLEDGVPSRFSGSGSGADYSLTISSLESEDFADYYCVQYAQFPYTFGGGTKLEIK

SEQ ID NO: 455 - protein, 114 aa, synthetic construct
  source 1..114: note = Antibody LK-RB2-28 VH
DVKLVESGGGLVKPGGSLKLSCAASGFTFSSYSMSWVRQTPEKRLEWVATISTGGSYTYYPDSMKGRFTISRDNAKNTLYLQMSSLKSEDTAMFYCTRGSMDYWGQGTSVTVSS

SEQ ID NO: 456 - protein, 107 aa, synthetic construct
  source 1..107: note = Antibody LK-RB2-28 VL
DILMTQSPSSMSVSLGDTVSIACHASQDISSNIGWLQQKPGNSFKGLIYHGTNLEDGVPSRFSGSGSGADYSLTISSLESEDFADYYCVQYAQFPYTFGGGTKLEIK

SEQ ID NO: 457 - protein, 122 aa, synthetic construct
  source 1..122: note = Antibody LK-RB2-29 VH
QVQLQQSGAELARPGASVKLSCKASGYTFTSYWMQWVKQRPGQGLEWIGAIYPGDGDTRYTQKFKGKATLTADKSSSTAYMQLSSLASEDSAVYYCARGYYGSSYADYFDYWGQGTTLTVSS

SEQ ID NO: 458 - protein, 107 aa, synthetic construct
  source 1..107: note = Antibody LK-RB2-29 VL
DIQMTQSPASLSASVGETVTITCRASGNIHNYLAWYQQKQGKSPQLLVYNAKTLADGVPSRFSGSGSGTQYSLKINSLQPEDFGSYYCQHFWSTPPTFGGGTKLEIK

SEQ ID NO: 459 - protein, 119 aa, synthetic construct
  source 1..119: note = Antibody LK-RB2-30 VH
EVQLVESGGGLVQPGGSLKLSCAASGFTFNTDGMSWVRQTPDKRLELVAIINSNGGSTYYPDSVKGRFTISRDNAKNTLYLQMSSLKSEDTAMYYCAREGSYGSWFAYWGQGTLVTVSA

SEQ ID NO: 460 - protein, 107 aa, synthetic construct
  source 1..107: note = Antibody LK-RB2-30 VL
DIQMTQSPASLSVSVGETVTITCRASENINNNLAWYHQKQGKSPQLLVYAATNLADGVPSRFSGSGSGTQYSLKINSLQSEDFGSYYCQHFWGTPFTFGGGTKLEIK

SEQ ID NO: 461 - protein, 115 aa, synthetic construct
  source 1..115: note = Antibody LK-RB2-31 VH
QVQLQQPGAELVKPGASMKLSCKASGYTFTTYYMYWVKQRPGLGLEWIGGINPSNGGTTFNEKFKNKATLTVDKSSSTAYMQLRSLTSEDSAVYYCTRWRFFDVWGAGTTVTVSS

SEQ ID NO: 462 - protein, 107 aa, synthetic construct
  source 1..107: note = Antibody LK-RB2-31 VL
SIVMTQTPEFLLVSAGDRVTITCKASQSVNNDVAWFQQKPGQSPKLLIYYASNRYTGVPDRFTGSGYGTDFTFTISTVQAEDLAIYFCQQDYSSPPTFGGATKLEIK

SEQ ID NO: 463 - protein, 117 aa, synthetic construct
  source 1..117: note = Antibody LK-RB2-32 VH
DVQLQESGPGLVKPSQSLSLTCSVTGYSITSGYYWNWIRQFPGNKLEWMGYISFDGSNHYNPSLRNRISITRDTFKNQFFLKLNSVTTEDTATFYCTRRDYNWFAYWGQGTLVTVSA

SEQ ID NO: 464 - protein, 107 aa, synthetic construct
  source 1..107: note = Antibody LK-RB2-32 VL
DIVLTQSPVTLSVTPGDSVSLSCRASQNINKNLHWYQQRSHESPRLLIKYVSQSISGIPSRFSGSGSGTDFTLSINSVETEDFGMYFCQQSNSWPLTFGAGTKLELR

SEQ ID NO: 465 - protein, 121 aa, synthetic construct
  source 1..121: note = Antibody LK-RB2-33 VH
EVQLQQSGVELVRPGALVKLSCKASGFNIKDYYMHWVKQRPEQGLEWIGWIDPEKGDTIYGPKFQGKASITADTSSNTAYLQLSSLTSEDTAVYYCSRGPNYGSSYAFAYWGQGTLVTVSA

SEQ ID NO: 466 - protein, 107 aa, synthetic construct
  source 1..107: note = Antibody LK-RB2-33 VL
DVVMTQSHKFMSTSVGDRVTITCKASQDVSNAVAWYQQKPGQSPKLLIYSASYRYTGVPDRFTGSGSGTDFTFTISSMQAEDLAVYYCQQHYSLPWTFGGGTKLEIK

SEQ ID NO: 467 - protein, 114 aa, synthetic construct
  source 1..114: note = Antibody LK-RB2-34 VH
DVKVVESGGGLVKPGGSLKLSCAASGFTFSSYSMSWVRQTPEKRLEWVATISTGSSYTYYSDSVKGRFTISRDNAKNTLYLQMSSLKSEDTAMFYCTRGSMDYWGQGTSVTVSS

SEQ ID NO: 468 - protein, 107 aa, synthetic construct
  source 1..107: note = Antibody LK-RB2-34 VL
DILMTQSPSSMSVSLGDTVSITCHASQDISSNIGWLQQKPGNSFKGLIYHGINLEDGVPSRFSGSGSGADYSLTISSLESEDFADYYCVQYAQFPYTFGGGTKLEIK

SEQ ID NO: 469 - protein, 119 aa, synthetic construct
  source 1..119: note = Antibody LK-RB2-35 VH
EVQLQQSGPELVKPGASVKISCKASGYTFTDYNMHWVKQSHGKSLEWIGSIYPYNGGSDYNQKFKSKATLTVDNSSSTAYMELRSLTSEDSAVYYCASELTGLYAMDYWGQGTSVTVSS

SEQ ID NO: 470 - protein, 107 aa, synthetic construct
  source 1..107: note = Antibody LK-RB2-35 VL
DIVMTQSPATLSVTPGDRVSLSCRASQSISDYLHWYQQKSHESPRLLIKYASQSISGIPSRFSGSGSGSDFTLSINSVEPEDVGVYYCQTGHSFPYTFGGGTKLEIK

SEQ ID NO: 471 - protein, 120 aa, synthetic construct
  source 1..120: note = Antibody LK-RB2-36 VH
QVQLQQSGAELVRPGASVKMSCKASGYTFTNYWIGWAKQRPGHGLEWIGDIYPGGTYTGYNEKFKGKATLTADKSSSTAYMQFSSLTSEDSAIYYCARRMDYDGYAMDYWGQGTSVTVSS

SEQ ID NO: 472 - protein, 107 aa, synthetic construct
  source 1..107: note = Antibody LK-RB2-36 VL
DIVLTQSPASLSVTPGDSVSLSCRASQSISNNLHWYQQKSHESPRLLIKYASQSISGIPSRFSGSGSGTDFTLSINSVETEDFGMYFCQQSNSWPLTFGAGTKLELK

SEQ ID NO: 473 - protein, 120 aa, synthetic construct
  source 1..120: note = Antibody LK-RB2-37 VH
QIQLVQSGPEVKKPGETVKISCKASGYTFTTYGMSWVKQAPGKGLKWMGWINTYSGVPTYADDFKGRFAFSLETSVSTAYLQINNLKNEDTSTYFCARRRDYDGYAMDYWGQGTSVTVSS

SEQ ID NO: 474 - protein, 113 aa, synthetic construct
  source 1..113: note = Antibody LK-RB2-37 VL
DIVMSQSPSSLAVSVGEKVTMSCKSSQSLLYSGNQKNYLAWYQQKPGQSPKLLIYWASARESGVPDRFTGSGSGTDFTLTISSVKAEDLAVYYCQQYYTYPFTFGSGTKLEIK

SEQ ID NO: 475 - protein, 118 aa, synthetic construct
  source 1..118: note = Antibody LK-RB2-38 VH
EVKLVESGGGLVQPGGSLKLSCAASGFSFSDYYMYWVRQTPEKRLEWVAYISNGGGNTYYPDTVKGRFTISRDNAKNSLYLQMSRLKSEDTGMYYCARDREDWYFDVWGTGTTVTVSS

SEQ ID NO: 476 - protein, 107 aa, synthetic construct
  source 1..107: note = Antibody LK-RB2-38 VL
DIVMTQSHTFMSTSVGDRVSITCKASQDVSTAVAWYQQKPGQSPKLLIYWASTRHTGVPDRFTGSGSGTDYTLTISSVQAEDLALYYCQQHYSTPRTFGGGTKLEIK

SEQ ID NO: 477 - protein, 118 aa, synthetic construct
  source 1..118: note = Antibody LK-RB2-39 VH
DVKLVESGGGSVQPGGSLKLSCAASGFTFSDYYMYWVRQTPEKRLEWVAYISNGGGNTYYPDTVKGRFTISRDNAKNTLYLQMSRLKSEDTAMYYCARSNWAWYFDVWGTGTTVTVSS

SEQ ID NO: 478 - protein, 113 aa, synthetic construct
  source 1..113: note = Antibody LK-RB2-39 VL
DIVMTQSPSSLAMSVGQKVTMSCKSSQSLLHSSNQKNYLAWYQQKPGQSPKLLVSFASTRESGVPDRFIGSGSGTDFTLTINSVQAEDLADYFCQQHYSTPLTFGAGTKLELK

SEQ ID NO: 479 - protein, 123 aa, synthetic construct
  source 1..123: note = Antibody LK-RB2-40 VH
DVQLQESGPGLVKPSQSLSLTCSVTGYSITSGYYWNWIRQFPGNKLEWMAYISYDGSNNYNPSLKNRISITRDTSKNQFFLKLNSVTTEDTATYYCAREEGTAQATLGYFDYWGQGTTLTVSS

SEQ ID NO: 480 - protein, 107 aa, synthetic construct
  source 1..107: note = Antibody LK-RB2-40 VL
DIQMTQSPASLSVSVGETVTITCRASENIYSNLAWYQQKQGKSPQLLVYAATNLADGVPSRFSGSGSGTQYSLKINSLQSEDFGSYYCQHFWDTPFTFGSGTKLEIK

SEQ ID NO: 481 - protein, 121 aa, synthetic construct
  source 1..121: note = Antibody LK-RB2-41 VH
EVQLQLSGPELVKPGASVKMSCKASGYTFTDYNMHWVKQSHGKSLEWIGYINPNNGGTTYNQKFKGKATLTVNKSSSTAYMELRSLTSEDSAVYYCARAPYYGSSYLLAYWGQGTLVTVSA

SEQ ID NO: 482 - protein, 107 aa, synthetic construct
  source 1..107: note = Antibody LK-RB2-41 VL
DIVMTQSHKFMSTSVGDRVSITCKASQDVSTAVAWYQQKPGQSPKLLIYSASYRYTGVPDRFTGSGSGTDFTFTISSVQAEDLAIYYCQQHYSIPWTFGGGTKLEIK

SEQ ID NO: 483 - protein, 118 aa, synthetic construct
  source 1..118: note = Antibody LK-RB2-42 VH
EFQLQQSGPELVKPGASVKISCKASGYSFTDYNMNWVKQSNGKSLDWIGIINPKYGTTAYNQKLRDKATLTVDQSSSTAYMQLNSLTSEDSAVYYCASLNWDEGFAYWGQGTLVTVSP

SEQ ID NO: 484 - protein, 112 aa, synthetic construct
  source 1..112: note = Antibody LK-RB2-42 VL
DVLMTQTPLSLPVSLGDQASISCRSSQSIVHSNGNTYLEWYLQKPGQPPKLLIYKVSNRFSGVPERFSGSGSGTDFTLKINRVEAEDLGVYYCFQGSLVPPTFGGGTKLEIK

SEQ ID NO: 485 - protein, 114 aa, synthetic construct
  source 1..114: note = Antibody LK-RB2-43 VH
EVQLVESGGDLVKPGGSLKLSCAASGFTFSSYGMSWVRQTPDKRLEWVATISGGGTYTYYPDSVKGRFTISRDNAKNTLYLQMSSLKSEDTAMYYCASGAMDYWGQGTSVTVSS

SEQ ID NO: 486 - protein, 106 aa, synthetic construct
  source 1..106: note = Antibody LK-RB2-43 VL
QIVLTQSPAILSASPGEKVTMTCSANSSVSSMYKYQQKPGSSPKPWIYGTSNLASGVPARFSGSGSGTSYSLTISSMEAADAATYYCQQYHSHPLTFGAGTKLELK

SEQ ID NO: 487 - protein, 114 aa, synthetic construct; identical to SEQ ID NO: 485
  source 1..114: note = Antibody LK-RB2-44 VH

SEQ ID NO: 488 - protein, 112 aa, synthetic construct
  source 1..112: note = Antibody LK-RB2-44 VL
DVVMTQTPLTLSVTIGQPASISCKSSQSLLDIDGKTFLNWLLQRPGQSPKRLIYLVSKLDSGVPDRFTGSGSGTDFTLKISRVEAEDLGVYYCWQGTHFPWTFGGGTKLEIK

SEQ ID NO: 489 - protein, 120 aa, synthetic construct
  source 1..120: note = Antibody LK-RB2-45 VH
QIQLVQSGPELKKPGETVKISCKASGYTFTTYGMSWVKQTPGKGLQWMGWINVYSGVPTYADDFKGRFAFSLEASASTAYLQINNLKNEDTASYFCARRGDYDGHYFDYWGQGTTLTVSS

SEQ ID NO: 490 - protein, 107 aa, synthetic construct
  source 1..107: note = Antibody LK-RB2-45 VL
DIVMTQSPATLSVTPGDRVSLSCRASQSISDYLNWYQQKSHESPRLLIKYVSQSISGIPSRFSGSGSGSDFTLSINSVEPEDIGVYYCQNGHNFPLTFGGGTKLELK

SEQ ID NO: 491 - protein, 117 aa, synthetic construct
  source 1..117: note = Antibody LK-RB2-46 VH
QVQMQQSDAELVKPGASVKISCKVSGYTFTDQIIHWMKQRPEQGLEWIGYIYPRDDSIMYNEKFKGKATLTADKSSSTANMQLNSLTSEDSAVYFCAYDAYWYFDVWGTGTTVIVSS

SEQ ID NO: 492 - protein, 107 aa, synthetic construct
  source 1..107: note = Antibody LK-RB2-46 VL
DIVMTQSHKFMSASVGDRVSITCKASQDVNTAVAWYRQDPGQSPKLLIYWASTRHTGVPDRFTGSGSGTDYTLTINNFQAEDLALYYCQQHYSPPLTFGAGTKLELK

SEQ ID NO: 493 - protein, 119 aa, synthetic construct
  source 1..119: note = Antibody LK-RB2-47 VH
QVQLKQSGPGLVQPSQSLSITCTVSGFSLISYGVHWVRQSPGKGLEWLGVIWSGGSTDYNAAFISRLSISKDNSKSQVFFKMNSLQADDTAIYYCARSFHYHGSTFDYWGQGTTLTVSS

SEQ ID NO: 494 - protein, 107 aa, synthetic construct
  source 1..107: note = Antibody LK-RB2-47 VL
DIQMTQTTSSLSASLGDRVIISCRASQDINNYLNWYQQKPDGTVKLLIYFTSRLHSGVPSSFSGSGSGTDYSLTINNLEQEDIATYFCQQVNTLPYTFGGGTKLEIK

SEQ ID NO: 495 - protein, 5 aa, synthetic construct
  source 1..5: note = Antibody LK-RB1-1 CDR-H1
NFGMS

SEQ ID NO: 496 - protein, 17 aa, synthetic construct
  source 1..17: note = Antibody LK-RB1-1 CDR-H2
WINTYSGVPTYADDFKG

SEQ ID NO: 497 - protein, 13 aa, synthetic construct
  source 1..13: note = Antibody LK-RB1-1 CDR-H3
RRRTFDGYLCFDY

SEQ ID NO: 498 - protein, 12 aa, synthetic construct
  source 1..12: note = Antibody LK-RB1-1 CDR-L1
TLSSQHSTYSID

SEQ ID NO: 499 - protein, 11 aa, synthetic construct
  source 1..11: note = Antibody LK-RB1-1 CDR-L2
LKTDGSHRTGD

SEQ ID NO: 500 - protein, 13 aa, synthetic construct
  source 1..13: note = Antibody LK-RB1-1 CDR-L3
GVGDTIKEQFVYV

SEQ ID NO: 501 - protein, 5 aa, synthetic construct
  source 1..5: note = Antibody LK-RB1-2 CDR-H1
TYGVS

SEQ ID NO: 502 - protein, 17 aa, synthetic construct
  source 1..17: note = Antibody LK-RB1-2 CDR-H2
WINTYSGVSTYSDDFKG

SEQ ID NO: 503 - protein, 13 aa, synthetic construct
  source 1..13: note = Antibody LK-RB1-2 CDR-H3
RRRTYDGYLSFDY

SEQ ID NO: 504 - protein, 11 aa, synthetic construct
  source 1..11: note = Antibody LK-RB1-2 CDR-L1
GASENIYGALN

SEQ ID NO: 505 - protein, 7 aa, synthetic construct
  source 1..7: note = Antibody LK-RB1-2 CDR-L2
GATNLAD

SEQ ID NO: 506 - protein, 9 aa, synthetic construct
  source 1..9: note = Antibody LK-RB1-2 CDR-L3
QNVLTTPWT

SEQ ID NO: 507 - protein, 5 aa, synthetic construct
  source 1..5: note = Antibody LK-RB1-3 CDR-H1
SYWIH

SEQ ID NO: 508 - protein, 17 aa, synthetic construct
  source 1..17: note = Antibody LK-RB1-3 CDR-H2
RINPYSSGTRYNEKFKN

SEQ ID NO: 509 - protein, 10 aa, synthetic construct
  source 1..10: note = Antibody LK-RB1-3 CDR-H3
FDGAPDYFDF

SEQ ID NO: 510 - protein, 11 aa, synthetic construct
  source 1..11: note = Antibody LK-RB1-3 CDR-L1
KASQNVGINVA

SEQ ID NO: 511 - protein, 7 aa, synthetic construct
  source 1..7: note = Antibody LK-RB1-3 CDR-L2
SASYRYS

SEQ ID NO: 512 - protein, 9 aa, synthetic construct
  source 1..9: note = Antibody LK-RB1-3 CDR-L3
QQYNNYPFT

SEQ ID NO: 513 - protein, 5 aa, synthetic construct
  source 1..5: note = Antibody LK-RB1-4 CDR-H1
SYWMH

SEQ ID NO: 514 - protein, 17 aa, synthetic construct
  source 1..17: note = Antibody LK-RB1-4 CDR-H2
RIDPHYSGTKYNEKFKN

SEQ ID NO: 515 - protein, 10 aa, synthetic construct
  source 1..10: note = Antibody LK-RB1-4 CDR-H3
FDGDPYYFDY

SEQ ID NO: 516 - protein, 11 aa, synthetic construct
  source 1..11: note = Antibody LK-RB1-4 CDR-L1
KASQNVGTNVA

SEQ ID NO: 517 - protein, 7 aa, synthetic construct
  source 1..7: note = Antibody LK-RB1-4 CDR-L2
SASYRYS

SEQ ID NO: 518 - protein, 9 aa, synthetic construct
  source 1..9: note = Antibody LK-RB1-4 CDR-L3
QQYNNNPFT

SEQ ID NO: 519 - protein, 5 aa, synthetic construct
  source 1..5: note = Antibody LK-RB1-5 CDR-H1
DFWIH

SEQ ID NO: 520 - protein, 17 aa, synthetic construct
  source 1..17: note = Antibody LK-RB1-5 CDR-H2
MIHPNSGSTNFNEKFRA

SEQ ID NO: 521 - protein, 11 aa, synthetic construct
  source 1..11: note = Antibody LK-RB1-5 CDR-H3
SGDGYSNPMDY

SEQ ID NO: 522 - protein, 10 aa, synthetic construct
  source 1..10: note = Antibody LK-RB1-5 CDR-L1
RASSSVSYMH

SEQ ID NO: 523 - protein, 7 aa, synthetic construct
  source 1..7: note = Antibody LK-RB1-5 CDR-L2
ATSNLAS

SEQ ID NO: 524 - protein, 9 aa, synthetic construct
  source 1..9: note = Antibody LK-RB1-5 CDR-L3
QQWRRSPYT

SEQ ID NO: 525 - protein, 5 aa, synthetic construct
  source 1..5: note = Antibody LK-RB1-6 CDR-H1
TYGMS

SEQ ID NO: 526 - protein, 17 aa, synthetic construct
  source 1..17: note = Antibody LK-RB1-6 CDR-H2
WINTFSGVPACADDFQG

SEQ ID NO: 527 - protein, 13 aa, synthetic construct
  source 1..13: note = Antibody LK-RB1-6 CDR-H3
RSYYGSRGWYFDV

SEQ ID NO: 528 - protein, 11 aa, synthetic construct
  source 1..11: note = Antibody LK-RB1-6 CDR-L1
TASEDVNTAVA

SEQ ID NO: 529 - protein, 7 aa, synthetic construct
  source 1..7: note = Antibody LK-RB1-6 CDR-L2
WASTRHT

SEQ ID NO: 530 - protein, 9 aa, synthetic construct
  source 1..9: note = Antibody LK-RB1-6 CDR-L3
QQHYNTPYT

SEQ ID NO: 531 - protein, 5 aa, synthetic construct
  source 1..5: note = Antibody LK-RB1-7 CDR-H1
TYGMS

SEQ ID NO: 532 - protein, 17 aa, synthetic construct
  source 1..17: note = Antibody LK-RB1-7 CDR-H2
WINTFSGVPACADDFQG

SEQ ID NO: 533 - protein, 13 aa, synthetic construct
  source 1..13: note = Antibody LK-RB1-7 CDR-H3
RSYYGSRGWYFDV

SEQ ID NO: 534 - protein, 11 aa, synthetic construct
  source 1..11: note = Antibody LK-RB1-7 CDR-L1
QASQDTTINLN

SEQ ID NO: 535 - protein, 7 aa, synthetic construct
  source 1..7: note = Antibody LK-RB1-7 CDR-L2
GASNLED

SEQ ID NO: 536 - protein, 9 aa, synthetic construct
  source 1..9: note = Antibody LK-RB1-7 CDR-L3
LQHTYLPRT

SEQ ID NO: 537 - protein, 5 aa, synthetic construct
  source 1..5: note = Antibody LK-RB1-8 CDR-H1
NYLIE

SEQ ID NO: 538 - protein, 17 aa, synthetic construct
  source 1..17: note = Antibody LK-RB1-8 CDR-H2
VINPGSDGSTYNEKFKG

SEQ ID NO: 539 - protein, 11 aa, synthetic construct
  source 1..11: note = Antibody LK-RB1-8 CDR-H3
GDYGRSYGMDY

SEQ ID NO: 540 - protein, 11 aa, synthetic construct
  source 1..11: note = Antibody LK-RB1-8 CDR-L1
KASQNVGTAVV

SEQ ID NO: 541 - protein, 7 aa, synthetic construct
  source 1..7: note = Antibody LK-RB1-8 CDR-L2
SASNRYT

SEQ ID NO: 542 - protein, 9 aa, synthetic construct
  source 1..9: note = Antibody LK-RB1-8 CDR-L3
QQYSSYMYT

SEQ ID NO: 543 - protein, 5 aa, synthetic construct
  source 1..5: note = Antibody LK-RB1-9, LK-RB1-9.h1, LK-RB1-9.h2, LK-RB1-9.h3 CDR-H1
TYGMS

SEQ ID NO: 544 - protein, 17 aa, synthetic construct
  source 1..17: note = Antibody LK-RB1-9, LK-RB1-9.h1, LK-RB1-9.h2, LK-RB1-9.h3 CDR-H2
WINTYSGVPTYADDFKG

SEQ ID NO: 545 - protein, 13 aa, synthetic construct
  source 1..13: note = Antibody LK-RB1-9 CDR-H3
RRRTYHGYLCFDY

SEQ ID NO: 546 - protein, 11 aa, synthetic construct
  source 1..11: note = Antibody LK-RB1-9, LK-RB1-9.h1, LK-RB1-9.h2, LK-RB1-9.h3 CDR-L1
ETSENIYGALN

SEQ ID NO: 547 - protein, 7 aa, synthetic construct
  source 1..7: note = Antibody LK-RB1-9 CDR-L2
GATNLAD

SEQ ID NO: 548 - protein, 9 aa, synthetic construct
  source 1..9: note = Antibody LK-RB1-9, LK-RB1-9.h1, LK-RB1-9.h2, LK-RB1-9.h3 CDR-L3
QNVLNTPWT

SEQ ID NO: 549 - protein, 7 aa, synthetic construct
  source 1..7: note = Antibody LK-RB1-10 CDR-H1
TSGTGVG

SEQ ID NO: 550 - protein, 16 aa, synthetic construct
  source 1..16: note = Antibody LK-RB1-10 CDR-H2
HIWWNDDKYYKPSLKR

SEQ ID NO: 551 - protein, 8 aa, synthetic construct
  source 1..8: note = Antibody LK-RB1-10 CDR-H3
TELGRGDY

SEQ ID NO: 552 - protein, 11 aa, synthetic construct
  source 1..11: note = Antibody LK-RB1-10 CDR-L1
RASQSIGTNIH

SEQ ID NO: 553 - protein, 7 aa, synthetic construct
  source 1..7: note = Antibody LK-RB1-10 CDR-L2
YASELIS

SEQ ID NO: 554 - protein, 9 aa, synthetic construct
  source 1..9: note = Antibody LK-RB1-10 CDR-L3
QQSYGWPYT

SEQ ID NO: 555 - protein, 7 aa, synthetic construct
  source 1..7: note = Antibody LK-RB1-11 CDR-H1
TSGMSVG

SEQ ID NO: 556 - protein, 16 aa, synthetic construct
  source 1..16: note = Antibody LK-RB1-11 CDR-H2
HIWWNDDKYYNPVLKG

SEQ ID NO: 557 - protein, 12 aa, synthetic construct
  source 1..12: note = Antibody LK-RB1-11 CDR-H3
YGNSHGYWYFDV

SEQ ID NO: 558 - protein, 15 aa, synthetic construct
  source 1..15: note = Antibody LK-RB1-11 CDR-L1
RASESVDSYGNNFIH

SEQ ID NO: 559 - protein, 7 aa, synthetic construct
  source 1..7: note = Antibody LK-RB1-11 CDR-L2
YASNLEA

SEQ ID NO: 560 - protein, 9 aa, synthetic construct
  source 1..9: note = Antibody LK-RB1-11 CDR-L3
QQNNEDPYT

SEQ ID NO: 561 - protein, 5 aa, synthetic construct
  source 1..5: note = Antibody LK-RB1-12 CDR-H1
TYWMD

SEQ ID NO: 562 - protein, 17 aa, synthetic construct
  source 1..17: note = Antibody LK-RB1-12 CDR-H2
QIYPGDGATNYNGKFKG

SEQ ID NO: 563 - protein, 11 aa, synthetic construct
  source 1..11: note = Antibody LK-RB1-12 CDR-H3
SRDYDGWFFDV

SEQ ID NO: 564 - protein, 11 aa, synthetic construct
  source 1..11: note = Antibody LK-RB1-12 CDR-L1
KASQDVSSAVA

SEQ ID NO: 565 - protein, 7 aa, synthetic construct
  source 1..7: note = Antibody LK-RB1-12 CDR-L2
WASTRHT

SEQ ID NO: 566 - protein, 9 aa, synthetic construct
  source 1..9: note = Antibody LK-RB1-12 CDR-L3
QQHYSTPWT

SEQ ID NO: 567 - protein, 7 aa, synthetic construct
  source 1..7: note = Antibody LK-RB1-13 CDR-H1
ISGTGIG

SEQ ID NO: 568 - protein, 16 aa, synthetic construct
  source 1..16: note = Antibody LK-RB1-13 CDR-H2
HIWWDDDKYYNPSLKS

SEQ ID NO: 569 - protein, 8 aa, synthetic construct
  source 1..8: note = Antibody LK-RB1-13 CDR-H3
TELGRGDF

SEQ ID NO: 570 - protein, 11 aa, synthetic construct
  source 1..11: note = Antibody LK-RB1-13 CDR-L1
RASQSTGTNIH

SEQ ID NO: 571 - protein, 7 aa, synthetic construct
  source 1..7: note = Antibody LK-RB1-13 CDR-L2
YGSESIS

SEQ ID NO: 572 - protein, 9 aa, synthetic construct
  source 1..9: note = Antibody LK-RB1-13 CDR-L3
QQSYSWPYT

SEQ ID NO: 573 - protein, 7 aa, synthetic construct
  source 1..7: note = Antibody LK-RB1-14 CDR-H1
TSGMGVG

SEQ ID NO: 574 - protein, 16 aa, synthetic construct
  source 1..16: note = Antibody LK-RB1-14 CDR-H2
HIWWDDDKYYNPSLKS

SEQ ID NO: 575 - protein, 8 aa, synthetic construct
  source 1..8: note = Antibody LK-RB1-14 CDR-H3
TELGRGDS

SEQ ID NO: 576 - protein, 11 aa, synthetic construct
  source 1..11: note = Antibody LK-RB1-14 CDR-L1
RASQSIGTSIH

SEQ ID NO: 577 - protein, 7 aa, synthetic construct
  source 1..7: note = Antibody LK-RB1-14 CDR-L2
YASESIS

SEQ ID NO: 578 - protein, 9 aa, synthetic construct
  source 1..9: note = Antibody LK-RB1-14 CDR-L3
QQSYSWPYT

SEQ ID NO: 579 - protein, 7 aa, synthetic construct
  source 1..7: note = Antibody LK-RB1-15 CDR-H1
ISGTGIG

SEQ ID NO: 580 - protein, 16 aa, synthetic construct
  source 1..16: note = Antibody LK-RB1-15 CDR-H2
HIWWDDDKYYNPSLKS

SEQ ID NO: 581 - protein, 8 aa, synthetic construct
  source 1..8: note = Antibody LK-RB1-15 CDR-H3
TELGRGDF

SEQ ID NO: 582 - protein, 11 aa, synthetic construct
  source 1..11: note = Antibody LK-RB1-15 CDR-L1
RASQSIGTNIH

SEQ ID NO: 583 - protein, 7 aa, synthetic construct
  source 1..7: note = Antibody LK-RB1-15 CDR-L2
YGSESIS

SEQ ID NO: 584 - protein, 9 aa, synthetic construct
  source 1..9: note = Antibody LK-RB1-15 CDR-L3
QQSYSWPYT

SEQ ID NO: 585 - protein, 5 aa, synthetic construct
  source 1..5: note = Antibody LK-RB1-16, LK-RB1-16.h1, LK-RB1-16.h2, LK-RB1-16.h3 CDR-H1
SNAIS

SEQ ID NO: 586 - protein, 16 aa, synthetic construct
  source 1..16: note = Antibody LK-RB1-16, LK-RB1-16.h1, LK-RB1-16.h2, LK-RB1-16.h3 CDR-H2
VIWGDGNTIYHSALRS

SEQ ID NO: 587 - protein, 10 aa, synthetic construct
  source 1..10: note = Antibody LK-RB1-16, LK-RB1-16.h1, LK-RB1-16.h2, LK-RB1-16.h3 CDR-H3
ANWDGYYFDY

SEQ ID NO: 588 - protein, 11 aa, synthetic construct
  source 1..11: note = Antibody LK-RB1-16, LK-RB1-16.h1, LK-RB1-16.h2, LK-RB1-16.h3 CDR-L1
HASQSVDFWLN

SEQ ID NO: 589 - protein, 7 aa, synthetic construct
  source 1..7: note = Antibody LK-RB1-16, LK-RB1-16.h1, LK-RB1-16.h2, LK-RB1-16.h3 CDR-L2
KASNLHT

SEQ ID NO: 590 - protein, 9 aa, synthetic construct
  source 1..9: note = Antibody LK-RB1-16, LK-RB1-16.h1, LK-RB1-16.h2, LK-RB1-16.h3 CDR-L3
QQGQSYPLT

SEQ ID NO: 591 - protein, 5 aa, synthetic construct
  source 1..5: note = Antibody LK-RB1-17 CDR-H1
NYWIH

SEQ ID NO: 592 - protein, 17 aa, synthetic construct
  source 1..17: note = Antibody LK-RB1-17 CDR-H2
RIAPNSGGTKYNEKFKT

SEQ ID NO: 593 - protein, 10 aa, synthetic construct
  source 1..10: note = Antibody LK-RB1-17 CDR-H3
LDGSPYYFDY

SEQ ID NO: 594 - protein, 11 aa, synthetic construct
  source 1..11: note = Antibody LK-RB1-17 CDR-L1
KASQNVDTNVA

SEQ ID NO: 595 - protein, 7 aa, synthetic construct
  source 1..7: note = Antibody LK-RB1-17 CDR-L2
SASYRYS

SEQ ID NO: 596 - protein, 9 aa, synthetic construct
  source 1..9: note = Antibody LK-RB1-17 CDR-L3
QQYNSYPYT

SEQ ID NO: 597 - protein, 5 aa, synthetic construct
  source 1..5: note = Antibody LK-RB1-18 CDR-H1
NSGVT

SEQ ID NO: 598 - protein, 16 aa, synthetic construct
  source 1..16: note = Antibody LK-RB1-18 CDR-H2
VIWGDGSTNYHSALIS

SEQ ID NO: 599 - protein, 10 aa, synthetic construct
  source 1..10: note = Antibody LK-RB1-18 CDR-H3
PHWDYYNMDY

SEQ ID NO: 600 - protein, 11 aa, synthetic construct
  source 1..11: note = Antibody LK-RB1-18 CDR-L1
RASQDISNYLN

SEQ ID NO: 601 - protein, 7 aa, synthetic construct
  source 1..7: note = Antibody LK-RB1-18 CDR-L2
FTSRLHS

SEQ ID NO: 602 - protein, 9 aa, synthetic construct
  source 1..9: note = Antibody LK-RB1-18 CDR-L3
QQGITLPLT

SEQ ID NO: 603 - protein, 5 aa, synthetic construct
  source 1..5: note = Antibody LK-RB1-19 CDR-H1
SYGVS

SEQ ID NO: 604 - protein, 16 aa, synthetic construct
  source 1..16: note = Antibody LK-RB1-19 CDR-H2
IIWGDGNTDYHSALIS

SEQ ID NO: 605 - protein, 10 aa, synthetic construct
  source 1..10: note = Antibody LK-RB1-19 CDR-H3
PHWEYYTMDY

SEQ ID NO: 606 - protein, 11 aa, synthetic construct
  source 1..11: note = Antibody LK-RB1-19 CDR-L1
RASQDISNFLN

SEQ ID NO: 607 - protein, 7 aa, synthetic construct
  source 1..7: note = Antibody LK-RB1-19 CDR-L2
STSRLHS

SEQ ID NO: 608 - protein, 9 aa, synthetic construct
  source 1..9: note = Antibody LK-RB1-19 CDR-L3
QQGNTLPLT

SEQ ID NO: 609 - protein, 5 aa, synthetic construct
  source 1..5: note = Antibody LK-RB1-20 CDR-H1
SNGVS

SEQ ID NO: 610 - protein, 16 aa, synthetic construct
  source 1..16: note = Antibody LK-RB1-20 CDR-H2
LIWGDGSTNYHSALIS

SEQ ID NO: 611 - protein, 10 aa, synthetic construct
  source 1..10: note = Antibody LK-RB1-20 CDR-H3
PNWDGYHFDY

SEQ ID NO: 612 - protein, 11 aa, synthetic construct
  source 1..11: note = Antibody LK-RB1-20 CDR-L1
HASQNIDFWLS

SEQ ID NO: 613 - protein, 7 aa, synthetic construct
  source 1..7: note = Antibody LK-RB1-20 CDR-L2
KASNLHT

SEQ ID NO: 614 - protein, 9 aa, synthetic construct
  source 1..9: note = Antibody LK-RB1-20 CDR-L3
QQGQSYPLT

SEQ ID NO: 615 - protein, 5 aa, synthetic construct
  source 1..5: note = Antibody LK-RB1-21 CDR-H1
DYWTT

SEQ ID NO: 616 - protein, 17 aa, synthetic construct
  source 1..17: note = Antibody LK-RB1-21 CDR-H2
DIYPVSGSTNYNEKFKN

SEQ ID NO: 617 - protein, 10 aa, synthetic construct
  source 1..10: note = Antibody LK-RB1-21 CDR-H3
ALGRRYYFDY

SEQ ID NO: 618 - protein, 11 aa, synthetic construct
  source 1..11: note = Antibody LK-RB1-21 CDR-L1
KASQDVSTAVA

SEQ ID NO: 619 - protein, 7 aa, synthetic construct
  source 1..7: note = Antibody LK-RB1-21 CDR-L2
SASYRYT

SEQ ID NO: 620 - protein, 9 aa, synthetic construct
  source 1..9: note = Antibody LK-RB1-21 CDR-L3
QQHYTNPVT

SEQ ID NO: 621 - protein, 5 aa, synthetic construct
  source 1..5: note = Antibody LK-RB1-22 CDR-H1
SYGVS

SEQ ID NO: 622 - protein, 16 aa, synthetic construct
  source 1..16: note = Antibody LK-RB1-22 CDR-H2
IIWGDGNTNYHSALTS

SEQ ID NO: 623 - protein, 10 aa, synthetic construct
  source 1..10: note = Antibody LK-RB1-22 CDR-H3
PHWDHYSMDY

SEQ ID NO: 624 - protein, 11 aa, synthetic construct
  source 1..11: note = Antibody LK-RB1-22 CDR-L1
RASQDISNFLN

SEQ ID NO: 625 - protein, 7 aa, synthetic construct
  source 1..7: note = Antibody LK-RB1-22 CDR-L2
YTSRLHS

SEQ ID NO: 626 - protein, 9 aa, synthetic construct
  source 1..9: note = Antibody LK-RB1-22 CDR-L3
QQGNTLPLT

SEQ ID NO: 627 - protein, 5 aa, synthetic construct
  source 1..5: note = Antibody LK-RB1-23 CDR-H1
NYWMD

SEQ ID NO: 628 - protein, 17 aa, synthetic construct
  source 1..17: note = Antibody LK-RB1-23 CDR-H2
NIHPFGNETHYNQKFKD

SEQ ID NO: 629 - protein, 8 aa, synthetic construct
  source 1..8: note = Antibody LK-RB1-23 CDR-H3
GLLLYFDV

SEQ ID NO: 630 - protein, 11 aa, synthetic construct
  source 1..11: note = Antibody LK-RB1-23 CDR-L1
RASENIYSNLA

SEQ ID NO: 631 - protein, 7 aa, synthetic construct
  source 1..7: note = Antibody LK-RB1-23 CDR-L2
GATNLAD

SEQ ID NO: 632 - protein, 9 aa, synthetic construct
  source 1..9: note = Antibody LK-RB1-23 CDR-L3
QHFWGIPWT

SEQ ID NO: 633 - protein, 5 aa, synthetic construct
  source 1..5: note = Antibody LK-RB1-24 CDR-H1
GYWMH

SEQ ID NO: 634 - protein, 17 aa, synthetic construct
  source 1..17: note = Antibody LK-RB1-24 CDR-H2
MIHPNNGGTIYNERFNG

SEQ ID NO: 635 - protein, 5 aa, synthetic construct
  source 1..5: note = Antibody LK-RB1-24 CDR-H3
WDLDY

SEQ ID NO: 636 - protein, 16 aa, synthetic construct
  source 1..16: note = Antibody LK-RB1-24 CDR-L1
KSSQSLLNSDGKTYLN

SEQ ID NO: 637 - protein, 7 aa, synthetic construct
  source 1..7: note = Antibody LK-RB1-24 CDR-L2
LVSKLGS

SEQ ID NO: 638 - protein, 9 aa, synthetic construct
  source 1..9: note = Antibody LK-RB1-24 CDR-L3
WQGTHFPWT

SEQ ID NO: 639 - protein, 5 aa, synthetic construct
  source 1..5: note = Antibody LK-RB1-25 CDR-H1
SYGMN

SEQ ID NO: 640 - protein, 17 aa, synthetic construct
  source 1..17: note = Antibody LK-RB1-25 CDR-H2
WINTYSGVPTYADDFKG

SEQ ID NO: 641 - protein, 12 aa, synthetic construct
  source 1..12: note = Antibody LK-RB1-25 CDR-H3
RGRDSVVGHFDV

SEQ ID NO: 642 - protein, 11 aa, synthetic construct
  source 1..11: note = Antibody LK-RB1-25 CDR-L1
KASQDVNTAVA

SEQ ID NO: 643 - protein, 7 aa, synthetic construct
  source 1..7: note = Antibody LK-RB1-25 CDR-L2
WASTRHT

SEQ ID NO: 644 - protein, 9 aa, synthetic construct
  source 1..9: note = Antibody LK-RB1-25 CDR-L3
QQHYTIPFT

SEQ ID NO: 645 - protein, 5 aa, synthetic construct
  source 1..5: note = Antibody LK-RB1-26 CDR-H1
SYGVS

SEQ ID NO: 646 - protein, 16 aa, synthetic construct
  source 1..16: note = Antibody LK-RB1-26 CDR-H2
IIWGDGSTNYHSALIS

SEQ ID NO: 647 - protein, 10 aa, synthetic construct
  source 1..10: note = Antibody LK-RB1-26 CDR-H3
PNWDYYAMDY

SEQ ID NO: 648 - protein, 11 aa, synthetic construct
  source 1..11: note = Antibody LK-RB1-26 CDR-L1
RASQDISNFLN

SEQ ID NO: 649 - protein, 7 aa, synthetic construct
  source 1..7: note = Antibody LK-RB1-26 CDR-L2
YTSTLHS

SEQ ID NO: 650 - protein, 9 aa, synthetic construct
  source 1..9: note = Antibody LK-RB1-26 CDR-L3
QQGNTLPLT

SEQ ID NO: 651 - protein, 5 aa, synthetic construct
  source 1..5: note = Antibody LK-RB1-27 CDR-H1
SHYIH

SEQ ID NO: 652 - protein, 17 aa, synthetic construct
  source 1..17: note = Antibody LK-RB1-27 CDR-H2
WIYPGDGHTKYHEKFKD

SEQ ID NO: 653 - protein, 8 aa, synthetic construct
  source 1..8: note = Antibody LK-RB1-27 CDR-H3
YSDSWFPY

SEQ ID NO: 654 - protein, 11 aa, synthetic construct
  source 1..11: note = Antibody LK-RB1-27 CDR-L1
RASENIYSNLA

SEQ ID NO: 655 - protein, 7 aa, synthetic construct
  source 1..7: note = Antibody LK-RB1-27 CDR-L2
AATNLAD

SEQ ID NO: 656 - protein, 9 aa, synthetic construct
  source 1..9: note = Antibody LK-RB1-27 CDR-L3
QHFWGSPWT

SEQ ID NO: 657 - protein, 5 aa, synthetic construct
  source 1..5: note = Antibody LK-RB1-28 CDR-H1
SHYIH

SEQ ID NO: 658 - protein, 17 aa, synthetic construct
  source 1..17: note = Antibody LK-RB1-28 CDR-H2
WIYPGDGHTKYHEKFKG

SEQ ID NO: 659 - protein, 8 aa, synthetic construct
  source 1..8: note = Antibody LK-RB1-28 CDR-H3
YSDSWFPY

SEQ ID NO: 660 - protein, 11 aa, synthetic construct
  source 1..11: note = Antibody LK-RB1-28 CDR-L1
RASENIYSNLA

SEQ ID NO: 661 - protein, 7 aa, synthetic construct
  source 1..7: note = Antibody LK-RB1-28 CDR-L2
AATNLAD

SEQ ID NO: 662 - protein, 9 aa, synthetic construct
  source 1..9: note = Antibody LK-RB1-28 CDR-L3
QHFWGSPWT

SEQ ID NO: 663 - protein, 5 aa, synthetic construct
  source 1..5: note = Antibody LK-RB1-29 CDR-H1
NSGVS

SEQ ID NO: 664 - protein, 16 aa, synthetic construct
  source 1..16: note = Antibody LK-RB1-29 CDR-H2
IIWGDGNTNYHSALIS

SEQ ID NO: 665 - protein, 10 aa, synthetic construct
  source 1..10: note = Antibody LK-RB1-29 CDR-H3
PNWDYYALDY

SEQ ID NO: 666 - protein, 11 aa, synthetic construct
  source 1..11: note = Antibody LK-RB1-29 CDR-L1
RASQDISNYLN

SEQ ID NO: 667 - protein, 7 aa, synthetic construct
  source 1..7: note = Antibody LK-RB1-29 CDR-L2
YTSRLHS

SEQ ID NO: 668 - protein, 9 aa, synthetic construct
  source 1..9: note = Antibody LK-RB1-29 CDR-L3
QQGISLPCT

SEQ ID NO: 669 - protein, 5 aa, synthetic construct
  source 1..5: note = Antibody LK-RB1-30 CDR-H1
SYGVS

SEQ ID NO: 670 - protein, 16 aa, synthetic construct
  source 1..16: note = Antibody LK-RB1-30 CDR-H2
VIWGDGNTNYHSALIS

SEQ ID NO: 671 - protein, 10 aa, synthetic construct
  source 1..10: note = Antibody LK-RB1-30 CDR-H3
PSWDYYAMDY

SEQ ID NO: 672 - protein, 11 aa, synthetic construct
  source 1..11: note = Antibody LK-RB1-30 CDR-L1
RASQDISNYVN

SEQ ID NO: 673 - protein, 7 aa, synthetic construct
  source 1..7: note = Antibody LK-RB1-30 CDR-L2
YTSRLHS

SEQ ID NO: 674 - protein, 9 aa, synthetic construct
  source 1..9: note = Antibody LK-RB1-30 CDR-L3
QQGITLPCT

SEQ ID NO: 675 - protein, 5 aa, synthetic construct
  source 1..5: note = Antibody LK-RB1-31 CDR-H1
SYGVS

SEQ ID NO: 676 - protein, 16 aa, synthetic construct
  source 1..16: note = Antibody LK-RB1-31 CDR-H2
IIWGDGSTNYHSALKS

SEQ ID NO: 677 - protein, 10 aa, synthetic construct
  source 1..10: note = Antibody LK-RB1-31 CDR-H3
PNWDYYAMDY

SEQ ID NO: 678 - protein, 11 aa, synthetic construct
  source 1..11: note = Antibody LK-RB1-31 CDR-L1
RASQDISNYLN

SEQ ID NO: 679 - protein, 7 aa, synthetic construct
  source 1..7: note = Antibody LK-RB1-31 CDR-L2
YTSRLHS

SEQ ID NO: 680 - protein, 9 aa, synthetic construct
  source 1..9: note = Antibody LK-RB1-31 CDR-L3
QQGITLPCT

SEQ ID NO: 681 - protein, 5 aa, synthetic construct
  source 1..5: note = Antibody LK-RB1-32 CDR-H1
TYLMH

SEQ ID NO: 682 - protein, 17 aa, synthetic construct
  source 1..17: note = Antibody LK-RB1-32 CDR-H2
YINPSNDFTKYNQKFKD

SEQ ID NO: 683 - protein, 8 aa, synthetic construct
  source 1..8: note = Antibody LK-RB1-32 CDR-H3
NSDYDPSY

SEQ ID NO: 684 - protein, 11 aa, synthetic construct
  source 1..11: note = Antibody LK-RB1-32 CDR-L1
RASENIYSSLA

SEQ ID NO: 685 - protein, 7 aa, synthetic construct
  source 1..7: note = Antibody LK-RB1-32 CDR-L2
AATNLAD

SEQ ID NO: 686 - protein, 8 aa, synthetic construct
  source 1..8: note = Antibody LK-RB1-32 CDR-L3
QHFWGTWA

SEQ ID NO: 687 - protein, 5 aa, synthetic construct
  source 1..5: note = Antibody LK-RB1-33 CDR-H1
SYGMN

SEQ ID NO: 688 - protein, 17 aa, synthetic construct
  source 1..17: note = Antibody LK-RB1-33 CDR-H2
WINTYSGVPTYGDDFKG

SEQ ID NO: 689 - protein, 12 aa, synthetic construct
  source 1..12: note = Antibody LK-RB1-33 CDR-H3
RGRDSVVGHFDV

SEQ ID NO: 690 - protein, 11 aa, synthetic construct
  source 1..11: note = Antibody LK-RB1-33 CDR-L1
KASQDVNTAVA

SEQ ID NO: 691 - protein, 7 aa, synthetic construct
  source 1..7: note = Antibody LK-RB1-33 CDR-L2
WASTRHT

SEQ ID NO: 692 - protein, 9 aa, synthetic construct
  source 1..9: note = Antibody LK-RB1-33 CDR-L3
QQHYTIPFT

SEQ ID NO: 693 - protein, 5 aa, synthetic construct
  source 1..5: note = Antibody LK-RB1-34 CDR-H1
EYTIH

SEQ ID NO: 694 - protein, 17 aa, synthetic construct
  source 1..17: note = Antibody LK-RB1-34 CDR-H2
WFYPGSDSIKYNEKFKD

SEQ ID NO: 695 - protein, 14 aa, synthetic construct
  source 1..14: note = Antibody LK-RB1-34 CDR-H3
HEGPYFTSQYYFDY

SEQ ID NO: 696 - protein, 11 aa, synthetic construct
  source 1..11: note = Antibody LK-RB1-34 CDR-L1
RASRYIGTSIH

SEQ ID NO: 697 - protein, 7 aa, synthetic construct
  source 1..7: note = Antibody LK-RB1-34 CDR-L2
YASESIS

SEQ ID NO: 698 - protein, 9 aa, synthetic construct
  source 1..9: note = Antibody LK-RB1-34 CDR-L3
QQSNTWPLT

SEQ ID NO: 699 - protein, 6 aa, synthetic construct
  source 1..6: note = Antibody LK-RB1-35 CDR-H1
RGYQWN

SEQ ID NO: 700 - protein, 16 aa, synthetic construct
  source 1..16: note = Antibody LK-RB1-35 CDR-H2
YISYDGSNNYNPSLKN

SEQ ID NO: 701 - protein, 8 aa, synthetic construct
  source 1..8: note = Antibody LK-RB1-35 CDR-H3
RSYNSIDY

SEQ ID NO: 702 - protein, 11 aa, synthetic construct
  source 1..11: note = Antibody LK-RB1-35 CDR-L1
RASQSISSNLH

SEQ ID NO: 703 - protein, 7 aa, synthetic construct
  source 1..7: note = Antibody LK-RB1-35 CDR-L2
YASQSIS

SEQ ID NO: 704 - protein, 9 aa, synthetic construct
  source 1..9: note = Antibody LK-RB1-35 CDR-L3
QQSNSWPLT

SEQ ID NO: 705 - protein, 6 aa, synthetic construct
  source 1..6: note = Antibody LK-RB1-36 CDR-H1
RGYQWN

SEQ ID NO: 706 - protein, 16 aa, synthetic construct
  source 1..16: note = Antibody LK-RB1-36 CDR-H2
YISYDGSNNYNPSLKN

SEQ ID NO: 707 - protein, 8 aa, synthetic construct
  source 1..8: note = Antibody LK-RB1-36 CDR-H3
RSYNSIDY

SEQ ID NO: 708 - protein, 11 aa, synthetic construct
  source 1..11: note = Antibody LK-RB1-36 CDR-L1
RASQSISNNLH

SEQ ID NO: 709 - protein, 7 aa, synthetic construct
  source 1..7: note = Antibody LK-RB1-36 CDR-L2
YASQSIS

SEQ ID NO: 710 - protein, 9 aa, synthetic construct
  source 1..9: note = Antibody LK-RB1-36 CDR-L3
QQSNSWPLT

SEQ ID NO: 711 - protein, 5 aa, synthetic construct
  source 1..5: note = Antibody LK-RB1-37 CDR-H1
SYWMH

SEQ ID NO: 712 - protein, 17 aa, synthetic construct
  source 1..17: note = Antibody LK-RB1-37 CDR-H2
RIDPNSGYARYNEKFKN

SEQ ID NO: 713 - protein, 12 aa, synthetic construct
  source 1..12: note = Antibody LK-RB1-37 CDR-H3
SFITTAEWFFDV

SEQ ID NO: 714 - protein, 11 aa, synthetic construct
  source 1..11: note = Antibody LK-RB1-37 CDR-L1
RASQSIGTSIH

SEQ ID NO: 715 - protein, 7 aa, synthetic construct
  source 1..7: note = Antibody LK-RB1-37 CDR-L2
YTSESIS

SEQ ID NO: 716 - protein, 9 aa, synthetic construct
  source 1..9: note = Antibody LK-RB1-37 CDR-L3
QQSNSWPHT

SEQ ID NO: 717 - protein, 6 aa, synthetic construct
  source 1..6: note = Antibody LK-RB1-38 CDR-H1
SGYQWN

SEQ ID NO: 718 - protein, 16 aa, synthetic construct
  source 1..16: note = Antibody LK-RB1-38 CDR-H2
YISFDGSNNYNPSLKN

SEQ ID NO: 719 - protein, 8 aa, synthetic construct
  source 1..8: note = Antibody LK-RB1-38 CDR-H3
RDYDGVAY

SEQ ID NO: 720 - protein, 11 aa, synthetic construct
  source 1..11: note = Antibody LK-RB1-38 CDR-L1
RASQNISNYLH

SEQ ID NO: 721 - protein, 7 aa, synthetic construct
  source 1..7: note = Antibody LK-RB1-38 CDR-L2
YASQSIS

SEQ ID NO: 722 - protein, 9 aa, synthetic construct
  source 1..9: note = Antibody LK-RB1-38 CDR-L3
QQSNSWPLT

SEQ ID NO: 723 - protein, 5 aa, synthetic construct
  source 1..5: note = Antibody LK-RB1-39 CDR-H1
DDYVH

SEQ ID NO: 724 - protein, 17 aa, synthetic construct
  source 1..17: note = Antibody LK-RB1-39 CDR-H2
WIDPENGDTGYASKFQD

SEQ ID NO: 725 - protein, 7 aa, synthetic construct
  source 1..7: note = Antibody LK-RB1-39 CDR-H3
TRWTGSY

SEQ ID NO: 726 - protein, 10 aa, synthetic construct
  source 1..10: note = Antibody LK-RB1-39 CDR-L1
SASSSVNYMH

SEQ ID NO: 727 - protein, 7 aa, synthetic construct
  source 1..7: note = Antibody LK-RB1-39 CDR-L2
STANLAS

SEQ ID NO: 728 - protein, 11 aa, synthetic construct
  source 1..11: note = Antibody LK-RB1-39 CDR-L3
QQRSSYPPTWT

SEQ ID NO: 729 - protein, 6 aa, synthetic construct
  source 1..6: note = Antibody LK-RB1-40 CDR-H1
SGYQWN

SEQ ID NO: 730 - protein, 16 aa, synthetic construct
  source 1..16: note = Antibody LK-RB1-40 CDR-H2
YISFDGSNNYNPSLKN

SEQ ID NO: 731 - protein, 8 aa, synthetic construct
  source 1..8: note = Antibody LK-RB1-40 CDR-H3
RDYDGVAY

SEQ ID NO: 732 - protein, 16 aa, synthetic construct
  source 1..16: note = Antibody LK-RB1-40 CDR-L1
RSSQSIVHSNGNTYLE

SEQ ID NO: 733 - protein, 7 aa, synthetic construct
  source 1..7: note = Antibody LK-RB1-40 CDR-L2
KVSNRFS

SEQ ID NO: 734 - protein, 9 aa, synthetic construct
  source 1..9: note = Antibody LK-RB1-40 CDR-L3
FQGSHVPWT

SEQ ID NO: 735 - protein, 6 aa, synthetic construct
  source 1..6: note = Antibody LK-RB1-41 CDR-H1
SGYQWN

SEQ ID NO: 736 - protein, 16 aa, synthetic construct
  source 1..16: note = Antibody LK-RB1-41 CDR-H2
YISFDGSNNYNPSLKN

SEQ ID NO: 737 - protein, 8 aa, synthetic construct
  source 1..8: note = Antibody LK-RB1-41 CDR-H3
RDYDGVAY

SEQ ID NO: 738 - protein, 11 aa, synthetic construct
  source 1..11: note = Antibody LK-RB1-41 CDR-L1
RASQNISNYLH

SEQ ID NO: 739 - protein, 7 aa, synthetic construct
  source 1..7: note = Antibody LK-RB1-41 CDR-L2
YASQSIS

SEQ ID NO: 740 - protein, 9 aa, synthetic construct
  source 1..9: note = Antibody LK-RB1-41 CDR-L3
QQSNSWPLT

SEQ ID NO: 741 - protein, 5 aa, synthetic construct
  source 1..5: note = Antibody LK-RB1-42 CDR-H1
SYWVY

SEQ ID NO: 742 - protein, 17 aa, synthetic construct
  source 1..17: note = Antibody LK-RB1-42 CDR-H2
MIHPNSGSTNFNEKFRY

SEQ ID NO: 743 - protein, 12 aa, synthetic construct
  source 1..12: note = Antibody LK-RB1-42 CDR-H3
SDDGYPYHAMDS

SEQ ID NO: 744 - protein, 11 aa, synthetic construct
  source 1..11: note = Antibody LK-RB1-42 CDR-L1
KASQNVGTNVA

SEQ ID NO: 745 - protein, 7 aa, synthetic construct
  source 1..7: note = Antibody LK-RB1-42 CDR-L2
SASYRFS

SEQ ID NO: 746 - protein, 9 aa, synthetic construct
  source 1..9: note = Antibody LK-RB1-42 CDR-L3
HQYKNYPLT

SEQ ID NO: 747 - protein, 5 aa, synthetic construct
  source 1..5: note = Antibody LK-RB1-43 CDR-H1
DYNMN

SEQ ID NO: 748 - protein, 17 aa, synthetic construct
  source 1..17: note = Antibody LK-RB1-43 CDR-H2
IIDPNYGTTTYNQKFKG

SEQ ID NO: 749 - protein, 9 aa, synthetic construct
  source 1..9: note = Antibody LK-RB1-43 CDR-H3
EGRYSWFAY

SEQ ID NO: 750 - protein, 11 aa, synthetic construct
  source 1..11: note = Antibody LK-RB1-43 CDR-L1
RASENINSNLA

SEQ ID NO: 751 - protein, 7 aa, synthetic construct
  source 1..7: note = Antibody LK-RB1-43 CDR-L2
AATNLAD

SEQ ID NO: 752 - protein, 9 aa, synthetic construct
  source 1..9: note = Antibody LK-RB1-43 CDR-L3
QHFWGTPFT

SEQ ID NO: 753 - protein, 5 aa, synthetic construct
  source 1..5: note = Antibody LK-RB1-44 CDR-H1
NYWMH

SEQ ID NO: 754 - protein, 17 aa, synthetic construct
  source 1..17: note = Antibody LK-RB1-44 CDR-H2
YVHPTSDYTKYNQKFKD

SEQ ID NO: 755 - protein, 12 aa, synthetic construct
  source 1..12: note = Antibody LK-RB1-44 CDR-H3
SSLYDTNYVFDS

SEQ ID NO: 756 - protein, 11 aa, synthetic construct
  source 1..11: note = Antibody LK-RB1-44 CDR-L1
RASQSIGRSIH

SEQ ID NO: 757 - protein, 7 aa, synthetic construct
  source 1..7: note = Antibody LK-RB1-44 CDR-L2
YASESIS

SEQ ID NO: 758 - protein, 9 aa, synthetic construct
  source 1..9: note = Antibody LK-RB1-44 CDR-L3
QQSNNWPFT

SEQ ID NO: 759 - protein, 5 aa, synthetic construct
  source 1..5: note = Antibody LK-RB1-45 CDR-H1
NFWMH

SEQ ID NO: 760 - protein, 17 aa, synthetic construct
  source 1..17: note = Antibody LK-RB1-45 CDR-H2
YVNPTSDYTKYNQKFKD

SEQ ID NO: 761 - protein, 12 aa, synthetic construct
  source 1..12: note = Antibody LK-RB1-45 CDR-H3
SSLYDTNYVFDS

SEQ ID NO: 762 - protein, 11 aa, synthetic construct
  source 1..11: note = Antibody LK-RB1-45 CDR-L1
RASQSIGRSIH

SEQ ID NO: 763 - protein, 7 aa, synthetic construct
  source 1..7: note = Antibody LK-RB1-45 CDR-L2
YASESIS

SEQ ID NO: 764 - protein, 9 aa, synthetic construct
  source 1..9: note = Antibody LK-RB1-45 CDR-L3
QQSNNWPFT

SEQ ID NO: 765 - protein, 5 aa, synthetic construct
  source 1..5: note = Antibody LK-RB1-46 CDR-H1
DYYMN

SEQ ID NO: 766 - protein, 17 aa, synthetic construct
  source 1..17: note = Antibody LK-RB1-46 CDR-H2
DIYPINGDISYNQKFKG

SEQ ID NO: 767 - protein, 11 aa, synthetic construct
  source 1..11: note = Antibody LK-RB1-46 CDR-H3
LAQTTSGGFDY

SEQ ID NO: 768 - protein, 15 aa, synthetic construct
  source 1..15: note = Antibody LK-RB1-46 CDR-L1
KASQSVSFAGTSLMH

SEQ ID NO: 769 - protein, 7 aa, synthetic construct
  source 1..7: note = Antibody LK-RB1-46 CDR-L2
RASNLKA

SEQ ID NO: 770 - protein, 10 aa, synthetic construct
  source 1..10: note = Antibody LK-RB1-46 CDR-L3
QQSREYPMYT

SEQ ID NO: 771 - protein, 5 aa, synthetic construct
  source 1..5: note = Antibody LK-RB1-47 CDR-H1
TYGMS

SEQ ID NO: 772 - protein, 17 aa, synthetic construct
  source 1..17: note = Antibody LK-RB1-47 CDR-H2
WINTYSGVPTYTDDFKG

SEQ ID NO: 773 - protein, 13 aa, synthetic construct
  source 1..13: note = Antibody LK-RB1-47 CDR-H3
RERDFYGNIFFPY

SEQ ID NO: 774 - protein, 11 aa, synthetic construct
  source 1..11: note = Antibody LK-RB1-47 CDR-L1
RASQDISNYLN

SEQ ID NO: 775 - protein, 7 aa, synthetic construct
  source 1..7: note = Antibody LK-RB1-47 CDR-L2
YTSRIHS

SEQ ID NO: 776 - protein, 9 aa, synthetic construct
  source 1..9: note = Antibody LK-RB1-47 CDR-L3
QQGHTLPWT

SEQ ID NO: 777 - protein, 5 aa, synthetic construct
  source 1..5: note = Antibody LK-RB1-48 CDR-H1
DYYMY

SEQ ID NO: 778 - protein, 17 aa, synthetic construct
  source 1..17: note = Antibody LK-RB1-48 CDR-H2
YISNGGGISFYPDTVKG

SEQ ID NO: 779 - protein, 10 aa, synthetic construct
  source 1..10: note = Antibody LK-RB1-48 CDR-H3
ATKEVGAMDY

SEQ ID NO: 780 - protein, 11 aa, synthetic construct
  source 1..11: note = Antibody LK-RB1-48 CDR-L1
KASQNVGTAVA

SEQ ID NO: 781 - protein, 7 aa, synthetic construct
  source 1..7: note = Antibody LK-RB1-48 CDR-L2
SASNRYT

SEQ ID NO: 782 - protein, 8 aa, synthetic construct
  source 1..8: note = Antibody LK-RB1-48 CDR-L3
QQYSSYRT

SEQ ID NO: 783 - protein, 5 aa, synthetic construct
  source 1..5: note = Antibody LK-RB1-49 CDR-H1
TYGMS

SEQ ID NO: 784 - protein, 17 aa, synthetic construct
  source 1..17: note = Antibody LK-RB1-49 CDR-H2
WINTYSGVPTYADDFKG

SEQ ID NO: 785 - protein, 12 aa, synthetic construct
  source 1..12: note = Antibody LK-RB1-49 CDR-H3
LFRSWDSYAMDY

SEQ ID NO: 786 - protein, 11 aa, synthetic construct
  source 1..11: note = Antibody LK-RB1-49 CDR-L1
KASQNVGTAVA

SEQ ID NO: 787 - protein, 7 aa, synthetic construct
  source 1..7: note = Antibody LK-RB1-49 CDR-L2
SPSNRYT

SEQ ID NO: 788 - protein, 9 aa, synthetic construct
  source 1..9: note = Antibody LK-RB1-49 CDR-L3
QQYSTYPLT

SEQ ID NO: 789 - protein, 5 aa, synthetic construct
  source 1..5: note = Antibody LK-RB1-50 CDR-H1
SYGVH

SEQ ID NO: 790 - protein, 16 aa, synthetic construct
  source 1..16: note = Antibody LK-RB1-50 CDR-H2
VIWRGGNTDYNAPFVS

SEQ ID NO: 791 - protein, 13 aa, synthetic construct
  source 1..13: note = Antibody LK-RB1-50 CDR-H3
NSLDVYSSWYFDV

SEQ ID NO: 792 - protein, 10 aa, synthetic construct
  source 1..10: note = Antibody LK-RB1-50 CDR-L1
RASSSVSFMH

SEQ ID NO: 793 - protein, 7 aa, synthetic construct
  source 1..7: note = Antibody LK-RB1-50 CDR-L2
ATSNLAS

SEQ ID NO: 794 - protein, 9 aa, synthetic construct
  source 1..9: note = Antibody LK-RB1-50 CDR-L3
QQWSSNPLT

SEQ ID NO: 795 - protein, 5 aa, synthetic construct
  source 1..5: note = Antibody LK-RB1-51 CDR-H1
EFPIH

SEQ ID NO: 796 - protein, 17 aa, synthetic construct
  source 1..17: note = Antibody LK-RB1-51 CDR-H2
WFYPGSDNMNYNEKFKD

SEQ ID NO: 797 - protein, 11 aa, synthetic construct
  source 1..11: note = Antibody LK-RB1-51 CDR-H3
HENAYFYAMDY

SEQ ID NO: 798 - protein, 10 aa, synthetic construct
  source 1..10: note = Antibody LK-RB1-51 CDR-L1
RASSSVNYMY

SEQ ID NO: 799 - protein, 7 aa, synthetic construct
  source 1..7: note = Antibody LK-RB1-51 CDR-L2
YTSNLAP

SEQ ID NO: 800 - protein, 9 aa, synthetic construct
  source 1..9: note = Antibody LK-RB1-51 CDR-L3
QQFTSSPFT

SEQ ID NO: 801 - protein, 5 aa, synthetic construct
  source 1..5: note = Antibody LK-RB1-52 CDR-H1
DYYMY

SEQ ID NO: 802 - protein, 17 aa, synthetic construct
  source 1..17: note = Antibody LK-RB1-52 CDR-H2
YISNGGDITYYPDTVKG

SEQ ID NO: 803 - protein, 10 aa, synthetic construct
  source 1..10: note = Antibody LK-RB1-52 CDR-H3
ATTEVGAMDH

SEQ ID NO: 804 - protein, 11 aa, synthetic construct
  source 1..11: note = Antibody LK-RB1-52 CDR-L1
KASQNVGTAVA

SEQ ID NO: 805 - protein, 7 aa, synthetic construct
  source 1..7: note = Antibody LK-RB1-52 CDR-L2
AASNRYT

SEQ ID NO: 806 - protein, 8 aa, synthetic construct
  source 1..8: note = Antibody LK-RB1-52 CDR-L3
QQYRSYRT

SEQ ID NO: 807 - protein, 5 aa, synthetic construct
  source 1..5: note = Antibody LK-RB1-53 CDR-H1
TYGMS

SEQ ID NO: 808 - protein, 17 aa, synthetic construct
  source 1..17: note = Antibody LK-RB1-53 CDR-H2
WINTNYGVPTYADDFKG

SEQ ID NO: 809 - protein, 14 aa, synthetic construct
  source 1..14: note = Antibody LK-RB1-53 CDR-H3
GTRRRGISYFAMDY

SEQ ID NO: 810 - protein, 11 aa, synthetic construct
  source 1..11: note = Antibody LK-RB1-53 CDR-L1
KASQNVGTAVA

SEQ ID NO: 811 - protein, 7 aa, synthetic construct
  source 1..7: note = Antibody LK-RB1-53 CDR-L2
SASNRYT

SEQ ID NO: 812 - protein, 9 aa, synthetic construct
  source 1..9: note = Antibody LK-RB1-53 CDR-L3
QQYSSYPLT

SEQ ID NO: 813 - protein, 6 aa, synthetic construct
  source 1..6: note = Antibody LK-RB1-54 CDR-H1
SGFYWN

SEQ ID NO: 814 - protein, 16 aa, synthetic construct
  source 1..16: note = Antibody LK-RB1-54 CDR-H2
HISYDGTNNYNPSLKN

SEQ ID NO: 815 - protein, 11 aa, synthetic construct
  source 1..11: note = Antibody LK-RB1-54 CDR-H3
GGYGGSYGFDY

SEQ ID NO: 816 - protein, 10 aa, synthetic construct
  source 1..10: note = Antibody LK-RB1-54 CDR-L1
RASSSVNYMY

SEQ ID NO: 817 - protein, 7 aa, synthetic construct
  source 1..7: note = Antibody LK-RB1-54 CDR-L2
YTSNLAP

SEQ ID NO: 818 - protein, 9 aa, synthetic construct
  source 1..9: note = Antibody LK-RB1-54 CDR-L3
QQFTSSPWT

SEQ ID NO: 819 - protein, 5 aa, synthetic construct
  source 1..5: note = Antibody LK-RB1-55 CDR-H1
DYYMY

SEQ ID NO: 820 - protein, 17 aa, synthetic construct
  source 1..17: note = Antibody LK-RB1-55 CDR-H2
YISNGGDITFYPDTVKG

SEQ ID NO: 821 - protein, 10 aa, synthetic construct
  source 1..10: note = Antibody LK-RB1-55 CDR-H3
ATTEVGAMDH

SEQ ID NO: 822 - protein, 11 aa, synthetic construct
  source 1..11: note = Antibody LK-RB1-55 CDR-L1
KASQNVGTAVA

SEQ ID NO: 823 - protein, 7 aa, synthetic construct
  source 1..7: note = Antibody LK-RB1-55 CDR-L2
AASNRYT

SEQ ID NO: 824 - protein, 8 aa, synthetic construct
  source 1..8: note = Antibody LK-RB1-55 CDR-L3
QQYSSYRT

SEQ ID NO: 825 - protein, 5 aa, synthetic construct
  source 1..5: note = Antibody LK-RB1-56 CDR-H1
NYGVH

SEQ ID NO: 826 - protein, 16 aa, synthetic construct
  source 1..16: note = Antibody LK-RB1-56 CDR-H2
VIWRGGSTDNNAAFMS

SEQ ID NO: 827 - protein, 13 aa, synthetic construct
  source 1..13: note = Antibody LK-RB1-56 CDR-H3
NSDFYGSSWYFDV

SEQ ID NO: 828 - protein, 10 aa, synthetic construct
  source 1..10: note = Antibody LK-RB1-56 CDR-L1
RASSSVSFMH

SEQ ID NO: 829 - protein, 7 aa, synthetic construct
  source 1..7: note = Antibody LK-RB1-56 CDR-L2
ATSNLAS

SEQ ID NO: 830 - protein, 9 aa, synthetic construct
  source 1..9: note = Antibody LK-RB1-56 CDR-L3
QQWSRNPPT

SEQ ID NO: 831 - protein, 5 aa, synthetic construct
  source 1..5: note = Antibody LK-RB1-57 CDR-H1
DYYMY

SEQ ID NO: 832 - protein, 17 aa, synthetic construct
  source 1..17: note = Antibody LK-RB1-57 CDR-H2
YISNGGDITFYPDTVKG

SEQ ID NO: 833 - protein, 10 aa, synthetic construct
  source 1..10: note = Antibody LK-RB1-57 CDR-H3
ATTEVGAMDH

SEQ ID NO: 834 - protein, 11 aa, synthetic construct
  source 1..11: note = Antibody LK-RB1-57 CDR-L1
KASQNVGTAVA

SEQ ID NO: 835 - protein, 7 aa, synthetic construct
  source 1..7: note = Antibody LK-RB1-57 CDR-L2
AASNRYT

SEQ ID NO: 836 - protein, 8 aa, synthetic construct
  source 1..8: note = Antibody LK-RB1-57 CDR-L3
QQYSSYRT

SEQ ID NO: 837 - protein, 5 aa, synthetic construct
  source 1..5: note = Antibody LK-RB1-58 CDR-H1
TYGVH

SEQ ID NO: 838 - protein, 16 aa, synthetic construct
  source 1..16: note = Antibody LK-RB1-58 CDR-H2
VIWRGGSTDYNAAFMS

SEQ ID NO: 839 - protein, 13 aa, synthetic construct
  source 1..13: note = Antibody LK-RB1-58 CDR-H3
NSLYYGSSWYFDV

SEQ ID NO: 840 - protein, 11 aa, synthetic construct
  source 1..11: note = Antibody LK-RB1-58 CDR-L1
KASQNVGTAVA

SEQ ID NO: 841 - protein, 7 aa, synthetic construct
  source 1..7: note = Antibody LK-RB1-58 CDR-L2
AASNRYT

SEQ ID NO: 842 - protein, 8 aa, synthetic construct
  source 1..8: note = Antibody LK-RB1-58 CDR-L3
QQYSSYRT

SEQ ID NO: 843 - protein, 5 aa, synthetic construct
  source 1..5: note = Antibody LK-RB1-59 CDR-H1
TYGMS

SEQ ID NO: 844 - protein, 17 aa, synthetic construct
  source 1..17: note = Antibody LK-RB1-59 CDR-H2
WINTYSGVPTYADDFKG

SEQ ID NO: 845 - protein, 14 aa, synthetic construct
  source 1..14: note = Antibody LK-RB1-59 CDR-H3
RRIYYGNKGYAMDY

SEQ ID NO: 846 - protein, 11 aa, synthetic construct
  source 1..11: note = Antibody LK-RB1-59 CDR-L1
RASENIYSNLV

SEQ ID NO: 847 - protein, 7 aa, synthetic construct
  source 1..7: note = Antibody LK-RB1-59 CDR-L2
TATNLAD

SEQ ID NO: 848 - protein, 9 aa, synthetic construct
  source 1..9: note = Antibody LK-RB1-59 CDR-L3
QHFWGTPWT

SEQ ID NO: 849 - protein, 5 aa, synthetic construct
  source 1..5: note = Antibody LK-RB2-1 CDR-H1
TYGMS

SEQ ID NO: 850 - protein, 17 aa, synthetic construct
  source 1..17: note = Antibody LK-RB2-1 CDR-H2
WINTYSGVSTYADDFKG

SEQ ID NO: 851 - protein, 11 aa, synthetic construct
  source 1..11: note = Antibody LK-RB2-1 CDR-H3
RYDYDGYAMDY

SEQ ID NO: 852 - protein, 17 aa, synthetic construct
  source 1..17: note = Antibody LK-RB2-1 CDR-L1
KSSQSLLFSSNQKNYLA

SEQ ID NO: 853 - protein, 7 aa, synthetic construct
  source 1..7: note = Antibody LK-RB2-1 CDR-L2
WASTRES

SEQ ID NO: 854 - protein, 9 aa, synthetic construct
  source 1..9: note = Antibody LK-RB2-1 CDR-L3
QQYDTYPPT

SEQ ID NO: 855 - protein, 5 aa, synthetic construct
  source 1..5: note = Antibody LK-RB2-2, LK-RB2-2.h1, LK-RB2-2.h2, LK-RB2-2.h3 CDR-H1
SYAMS

SEQ ID NO: 856 - protein, 17 aa, synthetic construct
  source 1..17: note = Antibody LK-RB2-2, LK-RB2-2.h1, LK-RB2-2.h2, LK-RB2-2.h3 CDR-H2
YISSGGDYFHYADTVKG

SEQ ID NO: 857 - protein, 15 aa, synthetic construct
  source 1..15: note = Antibody LK-RB2-2, LK-RB2-2.h1, LK-RB2-2.h2, LK-RB2-2.h3 CDR-H3
EGAHYDSSYLAWFAH

SEQ ID NO: 858 - protein, 11 aa, synthetic construct
  source 1..11: note = Antibody LK-RB2-2, LK-RB2-2.h1, LK-RB2-2.h2, LK-RB2-2.h3 CDR-L1
RASENIYSNLA

SEQ ID NO: 859 - protein, 7 aa, synthetic construct
  source 1..7: note = Antibody LK-RB2-2 CDR-L2
AATNLAD

SEQ ID NO: 860 - protein, 9 aa, synthetic construct
  source 1..9: note = Antibody LK-RB2-2, LK-RB2-2.h1, LK-RB2-2.h2, LK-RB2-2.h3 CDR-L3
QQFWDNPLT

SEQ ID NO: 861 - protein, 5 aa, synthetic construct
  source 1..5: note = Antibody LK-RB2-3 CDR-H1
DHTIH

SEQ ID NO: 862 - protein, 17 aa, synthetic construct
  source 1..17: note = Antibody LK-RB2-3 CDR-H2
YIYPRDGSTKENEKFTG

SEQ ID NO: 863 - protein, 5 aa, synthetic construct
  source 1..5: note = Antibody LK-RB2-3 CDR-H3
SGFDY

SEQ ID NO: 864 - protein, 11 aa, synthetic construct
  source 1..11: note = Antibody LK-RB2-3 CDR-L1
KASQDIKSYLS

SEQ ID NO: 865 - protein, 7 aa, synthetic construct
  source 1..7: note = Antibody LK-RB2-3 CDR-L2
RANRLVD

SEQ ID NO: 866 - protein, 9 aa, synthetic construct
  source 1..9: note = Antibody LK-RB2-3 CDR-L3
LQYDEFPYT

SEQ ID NO: 867 - protein, 5 aa, synthetic construct
  source 1..5: note = Antibody LK-RB2-4 CDR-H1
NYGMS

SEQ ID NO: 868 - protein, 17 aa, synthetic construct
  source 1..17: note = Antibody LK-RB2-4 CDR-H2
TISSGGSYTFYPDSVKG

SEQ ID NO: 869 - protein, 14 aa, synthetic construct
  source 1..14: note = Antibody LK-RB2-4 CDR-H3
DYYYGKTIEGYFDY

SEQ ID NO: 870 - protein, 11 aa, synthetic construct
  source 1..11: note = Antibody LK-RB2-4 CDR-L1
RANQDISNYLN

SEQ ID NO: 871 - protein, 7 aa, synthetic construct
  source 1..7: note = Antibody LK-RB2-4 CDR-L2
YTSRLHS

SEQ ID NO: 872 - protein, 7 aa, synthetic construct
  source 1..7: note = Antibody LK-RB2-4 CDR-L3
YTSRLHS

SEQ ID NO: 873 - protein, 5 aa, synthetic construct
  source 1..5: note = Antibody LK-RB2-5 CDR-H1
TYGMS

SEQ ID NO: 874 - protein, 17 aa, synthetic construct
  source 1..17: note = Antibody LK-RB2-5 CDR-H2
WINTYSGVPTYADDFKG

SEQ ID NO: 875 - protein, 12 aa, synthetic construct
  source 1..12: note = Antibody LK-RB2-5 CDR-H3
RDGYDGGGNFDY

SEQ ID NO: 876 - protein, 11 aa, synthetic construct
  source 1..11: note = Antibody LK-RB2-5 CDR-L1
RASQSISDYLH

SEQ ID NO: 877 - protein, 7 aa, synthetic construct
  source 1..7: note = Antibody LK-RB2-5 CDR-L2
YASQSIS

SEQ ID NO: 878 - protein, 9 aa, synthetic construct
  source 1..9: note = Antibody LK-RB2-5 CDR-L3
QNGHGFPWT

SEQ ID NO: 879 - protein, 5 aa, synthetic construct
  source 1..5: note = Antibody LK-RB2-6 CDR-H1
TYGMS

SEQ ID NO: 880 - protein, 17 aa, synthetic construct
  source 1..17: note = Antibody LK-RB2-6 CDR-H2
WINIYSGVPTYADDFKG

SEQ ID NO: 881 - protein, 11 aa, synthetic construct
  source 1..11: note = Antibody LK-RB2-6 CDR-H3
RGDYDSFYFDY

SEQ ID NO: 882 - protein, 17 aa, synthetic construct
  source 1..17: note = Antibody LK-RB2-6 CDR-L1
KSSQSLLYSYNQKNYLA

SEQ ID NO: 883 - protein, 7 aa, synthetic construct
  source 1..7: note = Antibody LK-RB2-6 CDR-L2
WSSTRES

SEQ ID NO: 884 - protein, 9 aa, synthetic construct
  source 1..9: note = Antibody LK-RB2-6 CDR-L3
QQCYNYPYT

SEQ ID NO: 885 - protein, 5 aa, synthetic construct
  source 1..5: note = Antibody LK-RB2-7 CDR-H1
SYAMS

SEQ ID NO: 886 - protein, 17 aa, synthetic construct
  source 1..17: note = Antibody LK-RB2-7 CDR-H2
YISSGGDHFYYADTVKG

SEQ ID NO: 887 - protein, 15 aa, synthetic construct
  source 1..15: note = Antibody LK-RB2-7 CDR-H3
EGASYDSNFLAWFAY

SEQ ID NO: 888 - protein, 11 aa, synthetic construct
  source 1..11: note = Antibody LK-RB2-7 CDR-L1
RASENIYSNLA

SEQ ID NO: 889 - protein, 7 aa, synthetic construct
  source 1..7: note = Antibody LK-RB2-7 CDR-L2
GATNLAD

SEQ ID NO: 890 - protein, 9 aa, synthetic construct
  source 1..9: note = Antibody LK-RB2-7 CDR-L3
QNFWDIPLT

SEQ ID NO: 891 - protein, 5 aa, synthetic construct
  source 1..5: note = Antibody LK-RB2-8 CDR-H1
SYGMG

SEQ ID NO: 892 - protein, 17 aa, synthetic construct
  source 1..17: note = Antibody LK-RB2-8 CDR-H2
WINTHSGVPTYADDFKG

SEQ ID NO: 893 - protein, 8 aa, synthetic construct
  source 1..8: note = Antibody LK-RB2-8 CDR-H3
YGRPWFPY

SEQ ID NO: 894 - protein, 11 aa, synthetic construct
  source 1..11: note = Antibody LK-RB2-8 CDR-L1
RTSENIYSYLA

SEQ ID NO: 895 - protein, 7 aa, synthetic construct
  source 1..7: note = Antibody LK-RB2-8 CDR-L2
NAKSLAE

SEQ ID NO: 896 - protein, 9 aa, synthetic construct
  source 1..9: note = Antibody LK-RB2-8 CDR-L3
QQHYETPLT

SEQ ID NO: 897 - protein, 6 aa, synthetic construct
  source 1..6: note = Antibody LK-RB2-9 CDR-H1
SGYYWN

SEQ ID NO: 898 - protein, 16 aa, synthetic construct
  source 1..16: note = Antibody LK-RB2-9 CDR-H2
YIGYDGNNNYNPSLKN

SEQ ID NO: 899 - protein, 12 aa, synthetic construct
  source 1..12: note = Antibody LK-RB2-9 CDR-H3
EGSTMVTSWFAY

SEQ ID NO: 900 - protein, 11 aa, synthetic construct
  source 1..11: note = Antibody LK-RB2-9 CDR-L1
RASENIYSNLA

SEQ ID NO: 901 - protein, 7 aa, synthetic construct
  source 1..7: note = Antibody LK-RB2-9 CDR-L2
GATNLAD

SEQ ID NO: 902 - protein, 9 aa, synthetic construct
  source 1..9: note = Antibody LK-RB2-9 CDR-L3
QHFWDTPYT

SEQ ID NO: 903 - protein, 5 aa, synthetic construct
  source 1..5: note = Antibody LK-RB2-10 CDR-H1
ANTVS

SEQ ID NO: 904 - protein, 17 aa, synthetic construct
  source 1..17: note = Antibody LK-RB2-10 CDR-H2
TISGGSDNTYCPDSVKG

SEQ ID NO: 905 - protein, 12 aa, synthetic construct
  source 1..12: note = Antibody LK-RB2-10 CDR-H3
HYYDGPNWYFDV

SEQ ID NO: 906 - protein, 11 aa, synthetic construct
  source 1..11: note = Antibody LK-RB2-10 CDR-L1
KASQNVDTAVA

SEQ ID NO: 907 - protein, 7 aa, synthetic construct
  source 1..7: note = Antibody LK-RB2-10 CDR-L2
SASNRYI

SEQ ID NO: 908 - protein, 9 aa, synthetic construct
  source 1..9: note = Antibody LK-RB2-10 CDR-L3
QQYSSYPLT

SEQ ID NO: 909 - protein, 5 aa, synthetic construct
  source 1..5: note = Antibody LK-RB2-11 CDR-H1
TYGMS

SEQ ID NO: 910 - protein, 17 aa, synthetic construct
  source 1..17: note = Antibody LK-RB2-11 CDR-H2
WINTYSGEPTYGDDFMG

SEQ ID NO: 911 - protein, 9 aa, synthetic construct
  source 1..9: note = Antibody LK-RB2-11 CDR-H3
YHYDDGMDY

SEQ ID NO: 912 - protein, 11 aa, synthetic construct
  source 1..11: note = Antibody LK-RB2-11 CDR-L1
KASQDINNYLS

SEQ ID NO: 913 - protein, 7 aa, synthetic construct
  source 1..7: note = Antibody LK-RB2-11 CDR-L2
RANRLVD

SEQ ID NO: 914 - protein, 9 aa, synthetic construct
  source 1..9: note = Antibody LK-RB2-11 CDR-L3
LQYDEFPWM

SEQ ID NO: 915 - protein, 5 aa, synthetic construct
  source 1..5: note = Antibody LK-RB2-12 CDR-H1
TYGMS

SEQ ID NO: 916 - protein, 17 aa, synthetic construct
  source 1..17: note = Antibody LK-RB2-12 CDR-H2
WINIYSGVPTYADDFKG

SEQ ID NO: 917 - protein, 11 aa, synthetic construct
  source 1..11: note = Antibody LK-RB2-12 CDR-H3
RKDYSGHCFDY

SEQ ID NO: 918 - protein, 15 aa, synthetic construct
  source 1..15: note = Antibody LK-RB2-12 CDR-L1
RASESVDTYGISFMH

SEQ ID NO: 919 - protein, 7 aa, synthetic construct
  source 1..7: note = Antibody LK-RB2-12 CDR-L2
RASNLES

SEQ ID NO: 920 - protein, 9 aa, synthetic construct
  source 1..9: note = Antibody LK-RB2-12 CDR-L3
QQSKKDPYT

SEQ ID NO: 921 - protein, 5 aa, synthetic construct
  source 1..5: note = Antibody LK-RB2-13 CDR-H1
TYGMS

SEQ ID NO: 922 - protein, 17 aa, synthetic construct
  source 1..17: note = Antibody LK-RB2-13 CDR-H2
WINTYSGVPTFADDFKG

SEQ ID NO: 923 - protein, 11 aa, synthetic construct
  source 1..11: note = Antibody LK-RB2-13 CDR-H3
RINWDGYAMDY

SEQ ID NO: 924 - protein, 17 aa, synthetic construct
  source 1..17: note = Antibody LK-RB2-13 CDR-L1
KSSQSLLYSGNQKSYLA

SEQ ID NO: 925 - protein, 7 aa, synthetic construct
  source 1..7: note = Antibody LK-RB2-13 CDR-L2
WASTRES

SEQ ID NO: 926 - protein, 9 aa, synthetic construct
  source 1..9: note = Antibody LK-RB2-13 CDR-L3
QQYYSYPYT

SEQ ID NO: 927 - protein, 5 aa, synthetic construct
  source 1..5: note = Antibody LK-RB2-14 CDR-H1
SYGVH

SEQ ID NO: 928 - protein, 16 aa, synthetic construct
  source 1..16: note = Antibody LK-RB2-14 CDR-H2
VIWRGGSTDYNAAFMS

SEQ ID NO: 929 - protein, 14 aa, synthetic construct
  source 1..14: note = Antibody LK-RB2-14 CDR-H3
NEGDSTVVAWYFDV

SEQ ID NO: 930 - protein, 11 aa, synthetic construct
  source 1..11: note = Antibody LK-RB2-14 CDR-L1
SASQGISNYLN

SEQ ID NO: 931 - protein, 7 aa, synthetic construct
  source 1..7: note = Antibody LK-RB2-14 CDR-L2
YTSSLHS

SEQ ID NO: 932 - protein, 9 aa, synthetic construct
  source 1..9: note = Antibody LK-RB2-14 CDR-L3
QQYSKLPLT

SEQ ID NO: 933 - protein, 5 aa, synthetic construct
  source 1..5: note = Antibody LK-RB2-15 CDR-H1
TYGMS

SEQ ID NO: 934 - protein, 17 aa, synthetic construct
  source 1..17: note = Antibody LK-RB2-15 CDR-H2
WINVYSGVPTYADDFKG

SEQ ID NO: 935 - protein, 11 aa, synthetic construct
  source 1..11: note = Antibody LK-RB2-15 CDR-H3
RKDYSGHCFDY

SEQ ID NO: 936 - protein, 15 aa, synthetic construct
  source 1..15: note = Antibody LK-RB2-15 CDR-L1
RASESVDTYGISFMH

SEQ ID NO: 937 - protein, 7 aa, synthetic construct
  source 1..7: note = Antibody LK-RB2-15 CDR-L2
RASNLES

SEQ ID NO: 938 - protein, 9 aa, synthetic construct
  source 1..9: note = Antibody LK-RB2-15 CDR-L3
QQSKKEPYT

SEQ ID NO: 939 - protein, 5 aa, synthetic construct
  source 1..5: note = Antibody LK-RB2-16 CDR-H1
TYGMG

SEQ ID NO: 940 - protein, 17 aa, synthetic construct
  source 1..17: note = Antibody LK-RB2-16 CDR-H2
WINTNSGAPTYGDDFKG

SEQ ID NO: 941 - protein, 8 aa, synthetic construct
  source 1..8: note = Antibody LK-RB2-16 CDR-H3
YGRPWFAY

SEQ ID NO: 942 - protein, 11 aa, synthetic construct
  source 1..11: note = Antibody LK-RB2-16 CDR-L1
RASENIYSYLA

SEQ ID NO: 943 - protein, 7 aa, synthetic construct
  source 1..7: note = Antibody LK-RB2-16 CDR-L2
NAKTLAE

SEQ ID NO: 944 - protein, 9 aa, synthetic construct
  source 1..9: note = Antibody LK-RB2-16 CDR-L3
QHHYNIPLT

SEQ ID NO: 945 - protein, 5 aa, synthetic construct
  source 1..5: note = Antibody LK-RB2-17 CDR-H1
NYGMS

SEQ ID NO: 946 - protein, 17 aa, synthetic construct
  source 1..17: note = Antibody LK-RB2-17 CDR-H2
WINTYSGVPTFADDFRG

SEQ ID NO: 947 - protein, 11 aa, synthetic construct
  source 1..11: note = Antibody LK-RB2-17 CDR-H3
RINWDGYAMDY

SEQ ID NO: 948 - protein, 17 aa, synthetic construct
  source 1..17: note = Antibody LK-RB2-17 CDR-L1
KSSQSLLYSGNQKSYLA

SEQ ID NO: 949 - protein, 7 aa, synthetic construct
  source 1..7: note = Antibody LK-RB2-17 CDR-L2
WASSRES

SEQ ID NO: 950 - protein, 9 aa, synthetic construct
  source 1..9: note = Antibody LK-RB2-17 CDR-L3
QQYYSFPYT

SEQ ID NO: 951 - protein, 5 aa, synthetic construct
  source 1..5: note = Antibody LK-RB2-18 CDR-H1
DYYMN

SEQ ID NO: 952 - protein, 17 aa, synthetic construct
  source 1..17: note = Antibody LK-RB2-18 CDR-H2
DINPNNGGTSYNQKFKG

SEQ ID NO: 953 - protein, 14 aa, synthetic construct
  source 1..14: note = Antibody LK-RB2-18 CDR-H3
SGTTVVGTYWYFDV

SEQ ID NO: 954 - protein, 11 aa, synthetic construct
  source 1..11: note = Antibody LK-RB2-18 CDR-L1
RASENIYSNLA

SEQ ID NO: 955 - protein, 7 aa, synthetic construct
  source 1..7: note = Antibody LK-RB2-18 CDR-L2
GATNLAD

SEQ ID NO: 956 - protein, 9 aa, synthetic construct
  source 1..9: note = Antibody LK-RB2-18 CDR-L3
QHFWDTPFT

SEQ ID NO: 957 - protein, 5 aa, synthetic construct
  source 1..5: note = Antibody LK-RB2-19 CDR-H1
SYWMH

SEQ ID NO: 958 - protein, 17 aa, synthetic construct
  source 1..17: note = Antibody LK-RB2-19 CDR-H2
MIHPNSGGTNYNEKFKS

SEQ ID NO: 959 - protein, 12 aa, synthetic construct
  source 1..12: note = Antibody LK-RB2-19 CDR-H3
CTVVALYYALDY

SEQ ID NO: 960 - protein, 10 aa, synthetic construct
  source 1..10: note = Antibody LK-RB2-19 CDR-L1
RATSRVSYIH

SEQ ID NO: 961 - protein, 7 aa, synthetic construct
  source 1..7: note = Antibody LK-RB2-19 CDR-L2
VTSNLAS

SEQ ID NO: 962 - protein, 9 aa, synthetic construct
  source 1..9: note = Antibody LK-RB2-19 CDR-L3
QQWSTNPLT

SEQ ID NO: 963 - protein, 5 aa, synthetic construct
  source 1..5: note = Antibody LK-RB2-20 CDR-H1
RYWIT

SEQ ID NO: 964 - protein, 17 aa, synthetic construct
  source 1..17: note = Antibody LK-RB2-20 CDR-H2
DIYPGSATTNYNEKFKS

SEQ ID NO: 965 - protein, 13 aa, synthetic construct
  source 1..13: note = Antibody LK-RB2-20 CDR-H3
SGDYSNSNYAMDY

SEQ ID NO: 966 - protein, 11 aa, synthetic construct
  source 1..11: note = Antibody LK-RB2-20 CDR-L1
KASENVGTYVS

SEQ ID NO: 967 - protein, 7 aa, synthetic construct
  source 1..7: note = Antibody LK-RB2-20 CDR-L2
GASNRNT

SEQ ID NO: 968 - protein, 9 aa, synthetic construct
  source 1..9: note = Antibody LK-RB2-20 CDR-L3
GQSYSYPFT

SEQ ID NO: 969 - protein, 5 aa, synthetic construct
  source 1..5: note = Antibody LK-RB2-21 CDR-H1
SYGMS

SEQ ID NO: 970 - protein, 17 aa, synthetic construct
  source 1..17: note = Antibody LK-RB2-21 CDR-H2
TISSGGSYTYYPDSLKG

SEQ ID NO: 971 - protein, 12 aa, synthetic construct
  source 1..12: note = Antibody LK-RB2-21 CDR-H3
PYYDYIFYAIDY

SEQ ID NO: 972 - protein, 11 aa, synthetic construct
  source 1..11: note = Antibody LK-RB2-21 CDR-L1
HASQNINVWLS

SEQ ID NO: 973 - protein, 7 aa, synthetic construct
  source 1..7: note = Antibody LK-RB2-21 CDR-L2
KASNLHT

SEQ ID NO: 974 - protein, 9 aa, synthetic construct
  source 1..9: note = Antibody LK-RB2-21 CDR-L3
QQGQSYPLT

SEQ ID NO: 975 - protein, 5 aa, synthetic construct
  source 1..5: note = Antibody LK-RB2-22 CDR-H1
TYGMS

SEQ ID NO: 976 - protein, 17 aa, synthetic construct
  source 1..17: note = Antibody LK-RB2-22 CDR-H2
WINIYSGVPTYADDFKG

SEQ ID NO: 977 - protein, 11 aa, synthetic construct
  source 1..11: note = Antibody LK-RB2-22 CDR-H3
RADYDGYYFDY

SEQ ID NO: 978 - protein, 11 aa, synthetic construct
  source 1..11: note = Antibody LK-RB2-22 CDR-L1
RASQSVTNDVA

SEQ ID NO: 979 - protein, 7 aa, synthetic construct
  source 1..7: note = Antibody LK-RB2-22 CDR-L2
YASNRYT

SEQ ID NO: 980 - protein, 9 aa, synthetic construct
  source 1..9: note = Antibody LK-RB2-22 CDR-L3
QQGYSSPFT

SEQ ID NO: 981 - protein, 5 aa, synthetic construct
  source 1..5: note = Antibody LK-RB2-23 CDR-H1
TYGMS

SEQ ID NO: 982 - protein, 17 aa, synthetic construct
  source 1..17: note = Antibody LK-RB2-23 CDR-H2
WINIYSGVPTYADDFKG

SEQ ID NO: 983 - protein, 11 aa, synthetic construct
  source 1..11: note = Antibody LK-RB2-23 CDR-H3
RGDYDGYYFDY

SEQ ID NO: 984 - protein, 11 aa, synthetic construct
  source 1..11: note = Antibody LK-RB2-23 CDR-L1
RASQSLSNGLH

SEQ ID NO: 985 - protein, 7 aa, synthetic construct
  source 1..7: note = Antibody LK-RB2-23 CDR-L2
YGSQSIS

SEQ ID NO: 986 - protein, 9 aa, synthetic construct
  source 1..9: note = Antibody LK-RB2-23 CDR-L3
QQSDSWPHT

SEQ ID NO: 987 - protein, 5 aa, synthetic construct
  source 1..5: note = Antibody LK-RB2-24, LK-RB2-24.h1, LK-RB2-24.h2, LK-RB2-24.h3 CDR-H1
TYGMS

SEQ ID NO: 988 - protein, 17 aa, synthetic construct
  source 1..17: note = Antibody LK-RB2-24, LK-RB2-24.h1, LK-RB2-24.h2, LK-RB2-24.h3 CDR-H2
WLNIYSGVPAYADDFKG

SEQ ID NO: 989 - protein, 11 aa, synthetic construct
  source 1..11: note = Antibody LK-RB2-24, LK-RB2-24.h1, LK-RB2-24.h2, LK-RB2-24.h3 CDR-H3
RENYDGYGMDY

SEQ ID NO: 990 - protein, 11 aa, synthetic construct
  source 1..11: note = Antibody LK-RB2-24, LK-RB2-24.h1, LK-RB2-24.h2, LK-RB2-24.h3 CDR-L1
RASQSISDYLH

SEQ ID NO: 991 - protein, 7 aa, synthetic construct
  source 1..7: note = Antibody LK-RB2-24, LK-RB2-24.h1, LK-RB2-24.h2, LK-RB2-24.h3 CDR-L2
YASQSIS

SEQ ID NO: 992 - protein, 9 aa, synthetic construct
  source 1..9: note = Antibody LK-RB2-24, LK-RB2-24.h1, LK-RB2-24.h2, LK-RB2-24.h3 CDR-L3
QNGHSFPYT

SEQ ID NO: 993 - protein, 5 aa, synthetic construct
  source 1..5: note = Antibody LK-RB2-25 CDR-H1
SYGVH

SEQ ID NO: 994 - protein, 16 aa, synthetic construct
  source 1..16: note = Antibody LK-RB2-25 CDR-H2
VIWAGGSTKYNSALMS

SEQ ID NO: 995 - protein, 10 aa, synthetic construct
  source 1..10: note = Antibody LK-RB2-25 CDR-H3
DQMVRSAMDY

SEQ ID NO: 996 - protein, 11 aa, synthetic construct
  source 1..11: note = Antibody LK-RB2-25 CDR-L1
RASQGIRGNLD

SEQ ID NO: 997 - protein, 7 aa, synthetic construct
  source 1..7: note = Antibody LK-RB2-25 CDR-L2
STSNLNS

SEQ ID NO: 998 - protein, 9 aa, synthetic construct
  source 1..9: note = Antibody LK-RB2-25 CDR-L3
LQRNASPYT

SEQ ID NO: 999 - protein, 5 aa, synthetic construct
  source 1..5: note = Antibody LK-RB2-26 CDR-H1
SYGVH

SEQ ID NO: 1000 - protein, 16 aa, synthetic construct
  source 1..16: note = Antibody LK-RB2-26 CDR-H2
VIWAGGSTDYNAAFIS

SEQ ID NO: 1001 - protein, 10 aa, synthetic construct
  source 1..10: note = Antibody LK-RB2-26 CDR-H3
DQMVRSAMDY

SEQ ID NO: 1002 - protein, 11 aa, synthetic construct
  source 1..11: note = Antibody LK-RB2-26 CDR-L1
RASQGIRGNLD

SEQ ID NO: 1003 - protein, 7 aa, synthetic construct
  source 1..7: note = Antibody LK-RB2-26 CDR-L2
STSNLNS

SEQ ID NO: 1004 - protein, 9 aa, synthetic construct
  source 1..9: note = Antibody LK-RB2-26 CDR-L3
LQRNASPYT

SEQ ID NO: 1005 - protein, 5 aa, synthetic construct
  source 1..5: note = Antibody LK-RB2-27 CDR-H1
SYSMS

SEQ ID NO: 1006 - protein, 17 aa, synthetic construct
  source 1..17: note = Antibody LK-RB2-27 CDR-H2
TISTGGSYTYYPDSVKG

SEQ ID NO: 1007 - protein, 5 aa, synthetic construct
  source 1..5: note = Antibody LK-RB2-27 CDR-H3
GSMDY

SEQ ID NO: 1008 - protein, 11 aa, synthetic construct
  source 1..11: note = Antibody LK-RB2-27 CDR-L1
HASQDISSNIG

SEQ ID NO: 1009 - protein, 7 aa, synthetic construct
  source 1..7: note = Antibody LK-RB2-27 CDR-L2
HGTNLED

SEQ ID NO: 1010 - protein, 9 aa, synthetic construct
  source 1..9: note = Antibody LK-RB2-27 CDR-L3
VQYAQFPYT

SEQ ID NO: 1011 - protein, 5 aa, synthetic construct
  source 1..5: note = Antibody LK-RB2-28 CDR-H1
SYSMS

SEQ ID NO: 1012 - protein, 17 aa, synthetic construct
  source 1..17: note = Antibody LK-RB2-28 CDR-H2
TISTGGSYTYYPDSMKG

SEQ ID NO: 1013 - protein, 5 aa, synthetic construct
  source 1..5: note = Antibody LK-RB2-28 CDR-H3
GSMDY

SEQ ID NO: 1014 - protein, 11 aa, synthetic construct
  source 1..11: note = Antibody LK-RB2-28 CDR-L1
HASQDISSNIG

SEQ ID NO: 1015 - protein, 7 aa, synthetic construct
  source 1..7: note = Antibody LK-RB2-28 CDR-L2
HGTNLED

SEQ ID NO: 1016 - protein, 9 aa, synthetic construct
  source 1..9: note = Antibody LK-RB2-28 CDR-L3
VQYAQFPYT

SEQ ID NO: 1017 - protein, 5 aa, synthetic construct
  source 1..5: note = Antibody LK-RB2-29 CDR-H1
SYWMQ

SEQ ID NO: 1018 - protein, 17 aa, synthetic construct
  source 1..17: note = Antibody LK-RB2-29 CDR-H2
AIYPGDGDTRYTQKFKG

SEQ ID NO: 1019 - protein, 13 aa, synthetic construct
  source 1..13: note = Antibody LK-RB2-29 CDR-H3
GYYGSSYADYFDY

SEQ ID NO: 1020 - protein, 11 aa, synthetic construct
  source 1..11: note = Antibody LK-RB2-29 CDR-L1
RASGNIHNYLA

SEQ ID NO: 1021 - protein, 7 aa, synthetic construct
  source 1..7: note = Antibody LK-RB2-29 CDR-L2
NAKTLAD

SEQ ID NO: 1022 - protein, 9 aa, synthetic construct
  source 1..9: note = Antibody LK-RB2-29 CDR-L3
QHFWSTPPT

SEQ ID NO: 1023 - protein, 5 aa, synthetic construct
  source 1..5: note = Antibody LK-RB2-30 CDR-H1
TDGMS

SEQ ID NO: 1024 - protein, 17 aa, synthetic construct
  source 1..17: note = Antibody LK-RB2-30 CDR-H2
IINSNGGSTYYPDSVKG

SEQ ID NO: 1025 - protein, 10 aa, synthetic construct
  source 1..10: note = Antibody LK-RB2-30 CDR-H3
EGSYGSWFAY

SEQ ID NO: 1026 - protein, 11 aa, synthetic construct
  source 1..11: note = Antibody LK-RB2-30 CDR-L1
RASENINNNLA

SEQ ID NO: 1027 - protein, 7 aa, synthetic construct
  source 1..7: note = Antibody LK-RB2-30 CDR-L2
AATNLAD

SEQ ID NO: 1028 - protein, 9 aa, synthetic construct
  source 1..9: note = Antibody LK-RB2-30 CDR-L3
QHFWGTPFT

SEQ ID NO: 1029 - protein, 5 aa, synthetic construct
  source 1..5: note = Antibody LK-RB2-31 CDR-H1
TYYMY

SEQ ID NO: 1030 - protein, 17 aa, synthetic construct
  source 1..17: note = Antibody LK-RB2-31 CDR-H2
GINPSNGGTTFNEKFKN

SEQ ID NO: 1031 - protein, 6 aa, synthetic construct
  source 1..6: note = Antibody LK-RB2-31 CDR-H3
WRFFDV

SEQ ID NO: 1032 - protein, 11 aa, synthetic construct
  source 1..11: note = Antibody LK-RB2-31 CDR-L1
KASQSVNNDVA

SEQ ID NO: 1033 - protein, 7 aa, synthetic construct
  source 1..7: note = Antibody LK-RB2-31 CDR-L2
YASNRYT

SEQ ID NO: 1034 - protein, 9 aa, synthetic construct
  source 1..9: note = Antibody LK-RB2-31 CDR-L3
QQDYSSPPT

SEQ ID NO: 1035 - protein, 6 aa, synthetic construct
  source 1..6: note = Antibody LK-RB2-32 CDR-H1
SGYYWN

SEQ ID NO: 1036 - protein, 16 aa, synthetic construct
  source 1..16: note = Antibody LK-RB2-32 CDR-H2
YISFDGSNHYNPSLRN

SEQ ID NO: 1037 - protein, 8 aa, synthetic construct
  source 1..8: note = Antibody LK-RB2-32 CDR-H3
RDYNWFAY

SEQ ID NO: 1038 - protein, 11 aa, synthetic construct
  source 1..11: note = Antibody LK-RB2-32 CDR-L1
RASQNINKNLH

SEQ ID NO: 1039 - protein, 7 aa, synthetic construct
  source 1..7: note = Antibody LK-RB2-32 CDR-L2
YVSQSIS

SEQ ID NO: 1040 - protein, 9 aa, synthetic construct
  source 1..9: note = Antibody LK-RB2-32 CDR-L3
QQSNSWPLT

SEQ ID NO: 1041 - protein, 5 aa, synthetic construct
  source 1..5: note = Antibody LK-RB2-33 CDR-H1
DYYMH

SEQ ID NO: 1042 - protein, 17 aa, synthetic construct
  source 1..17: note = Antibody LK-RB2-33 CDR-H2
WIDPEKGDTIYGPKFQG

SEQ ID NO: 1043 - protein, 12 aa, synthetic construct
  source 1..12: note = Antibody LK-RB2-33 CDR-H3
GPNYGSSYAFAY

SEQ ID NO: 1044 - protein, 11 aa, synthetic construct
  source 1..11: note = Antibody LK-RB2-33 CDR-L1
KASQDVSNAVA

SEQ ID NO: 1045 - protein, 7 aa, synthetic construct
  source 1..7: note = Antibody LK-RB2-33 CDR-L2
SASYRYT

SEQ ID NO: 1046 - protein, 9 aa, synthetic construct
  source 1..9: note = Antibody LK-RB2-33 CDR-L3
QQHYSLPWT

SEQ ID NO: 1047 - protein, 5 aa, synthetic construct
  source 1..5: note = Antibody LK-RB2-34 CDR-H1
SYSMS

SEQ ID NO: 1048 - protein, 17 aa, synthetic construct
  source 1..17: note = Antibody LK-RB2-34 CDR-H2
TISTGSSYTYYSDSVKG

SEQ ID NO: 1049 - protein, 5 aa, synthetic construct
  source 1..5: note = Antibody LK-RB2-34 CDR-H3
GSMDY

SEQ ID NO: 1050 - protein, 11 aa, synthetic construct
  source 1..11: note = Antibody LK-RB2-34 CDR-L1
HASQDISSNIG

SEQ ID NO: 1051 - protein, 7 aa, synthetic construct
  source 1..7: note = Antibody LK-RB2-34 CDR-L2
HGINLED

SEQ ID NO: 1052 - protein, 9 aa, synthetic construct
  source 1..9: note = Antibody LK-RB2-34 CDR-L3
VQYAQFPYT

SEQ ID NO: 1053 - protein, 5 aa, synthetic construct
  source 1..5: note = Antibody LK-RB2-35 CDR-H1
DYNMH

SEQ ID NO: 1054 - protein, 17 aa, synthetic construct
  source 1..17: note = Antibody LK-RB2-35 CDR-H2
SIYPYNGGSDYNQKFKS

SEQ ID NO: 1055 - protein, 10 aa, synthetic construct
  source 1..10: note = Antibody LK-RB2-35 CDR-H3
ELTGLYAMDY

SEQ ID NO: 1056 - protein, 11 aa, synthetic construct
  source 1..11: note = Antibody LK-RB2-35 CDR-L1
RASQSISDYLH

SEQ ID NO: 1057 - protein, 7 aa, synthetic construct
  source 1..7: note = Antibody LK-RB2-35 CDR-L2
YASQSIS

SEQ ID NO: 1058 - protein, 9 aa, synthetic construct
  source 1..9: note = Antibody LK-RB2-35 CDR-L3
QTGHSFPYT

SEQ ID NO: 1059 - protein, 5 aa, synthetic construct
  source 1..5: note = Antibody LK-RB2-36 CDR-H1
NYWIG

SEQ ID NO: 1060 - protein, 17 aa, synthetic construct
  source 1..17: note = Antibody LK-RB2-36 CDR-H2
DIYPGGTYTGYNEKFKG

SEQ ID NO: 1061 - protein, 11 aa, synthetic construct
  source 1..11: note = Antibody LK-RB2-36 CDR-H3
RMDYDGYAMDY

SEQ ID NO: 1062 - protein, 11 aa, synthetic construct
  source 1..11: note = Antibody LK-RB2-36 CDR-L1
RASQSISNNLH

SEQ ID NO: 1063 - protein, 7 aa, synthetic construct
  source 1..7: note = Antibody LK-RB2-36 CDR-L2
YASQSIS

SEQ ID NO: 1064 - protein, 9 aa, synthetic construct
  source 1..9: note = Antibody LK-RB2-36 CDR-L3
QQSNSWPLT

SEQ ID NO: 1065 - protein, 5 aa, synthetic construct
  source 1..5: note = Antibody LK-RB2-37 CDR-H1
TYGMS

SEQ ID NO: 1066 - protein, 17 aa, synthetic construct
  source 1..17: note = Antibody LK-RB2-37 CDR-H2
WINTYSGVPTYADDFKG

SEQ ID NO: 1067 - protein, 11 aa, synthetic construct
  source 1..11: note = Antibody LK-RB2-37 CDR-H3
RRDYDGYAMDY

SEQ ID NO: 1068 - protein, 17 aa, synthetic construct
  source 1..17: note = Antibody LK-RB2-37 CDR-L1
KSSQSLLYSGNQKNYLA

SEQ ID NO: 1069 - protein, 7 aa, synthetic construct
  source 1..7: note = Antibody LK-RB2-37 CDR-L2
WASARES

SEQ ID NO: 1070 - protein, 9 aa, synthetic construct
  source 1..9: note = Antibody LK-RB2-37 CDR-L3
QQYYTYPFT

SEQ ID NO: 1071 - protein, 5 aa, synthetic construct
  source 1..5: note = Antibody LK-RB2-38 CDR-H1
DYYMY

SEQ ID NO: 1072 - protein, 17 aa, synthetic construct
  source 1..17: note = Antibody LK-RB2-38 CDR-H2
YISNGGGNTYYPDTVKG

SEQ ID NO: 1073 - protein, 9 aa, synthetic construct
  source 1..9: note = Antibody LK-RB2-38 CDR-H3
DREDWYFDV

SEQ ID NO: 1074 - protein, 11 aa, synthetic construct
  source 1..11: note = Antibody LK-RB2-38 CDR-L1
KASQDVSTAVA

SEQ ID NO: 1075 - protein, 7 aa, synthetic construct
  source 1..7: note = Antibody LK-RB2-38 CDR-L2
WASTRHT

SEQ ID NO: 1076 - protein, 9 aa, synthetic construct
  source 1..9: note = Antibody LK-RB2-38 CDR-L3
QQHYSTPRT

SEQ ID NO: 1077 - protein, 5 aa, synthetic construct
  source 1..5: note = Antibody LK-RB2-39 CDR-H1
DYYMY

SEQ ID NO: 1078 - protein, 17 aa, synthetic construct
  source 1..17: note = Antibody LK-RB2-39 CDR-H2
YISNGGGNTYYPDTVKG

SEQ ID NO: 1079 - protein, 9 aa, synthetic construct
  source 1..9: note = Antibody LK-RB2-39 CDR-H3
SNWAWYFDV

SEQ ID NO: 1080 - protein, 17 aa, synthetic construct
  source 1..17: note = Antibody LK-RB2-39 CDR-L1
KSSQSLLHSSNQKNYLA

SEQ ID NO: 1081 - protein, 7 aa, synthetic construct
  source 1..7: note = Antibody LK-RB2-39 CDR-L2
FASTRES

SEQ ID NO: 1082 - protein, 9 aa, synthetic construct
  source 1..9: note = Antibody LK-RB2-39 CDR-L3
QQHYSTPLT

SEQ ID NO: 1083 - protein, 6 aa, synthetic construct
  source 1..6: note = Antibody LK-RB2-40 CDR-H1
SGYYWN

SEQ ID NO: 1084 - protein, 16 aa, synthetic construct
  source 1..16: note = Antibody LK-RB2-40 CDR-H2
YISYDGSNNYNPSLKN

SEQ ID NO: 1085 - protein, 14 aa, synthetic construct
  source 1..14: note = Antibody LK-RB2-40 CDR-H3
EEGTAQATLGYFDY

SEQ ID NO: 1086 - protein, 11 aa, synthetic construct
  source 1..11: note = Antibody LK-RB2-40 CDR-L1
RASENIYSNLA

SEQ ID NO: 1087 - protein, 7 aa, synthetic construct
  source 1..7: note = Antibody LK-RB2-40 CDR-L2
AATNLAD

SEQ ID NO: 1088 - protein, 9 aa, synthetic construct
  source 1..9: note = Antibody LK-RB2-40 CDR-L3
QHFWDTPFT

SEQ ID NO: 1089 - protein, 5 aa, synthetic construct
  source 1..5: note = Antibody LK-RB2-41 CDR-H1
DYNMH

SEQ ID NO: 1090 - protein, 17 aa, synthetic construct
  source 1..17: note = Antibody LK-RB2-41 CDR-H2
YINPNNGGTTYNQKFKG

SEQ ID NO: 1091 - protein, 12 aa, synthetic construct
  source 1..12: note = Antibody LK-RB2-41 CDR-H3
APYYGSSYLLAY

SEQ ID NO: 1092 - protein, 11 aa, synthetic construct
  source 1..11: note = Antibody LK-RB2-41 CDR-L1
KASQDVSTAVA

SEQ ID NO: 1093 - protein, 7 aa, synthetic construct
  source 1..7: note = Antibody LK-RB2-41 CDR-L2
SASYRYT

SEQ ID NO: 1094 - protein, 9 aa, synthetic construct
  source 1..9: note = Antibody LK-RB2-41 CDR-L3
QQHYSIPWT

SEQ ID NO: 1095 - protein, 5 aa, synthetic construct
  source 1..5: note = Antibody LK-RB2-42 CDR-H1
DYNMN

SEQ ID NO: 1096 - protein, 17 aa, synthetic construct
  source 1..17: note = Antibody LK-RB2-42 CDR-H2
IINPKYGTTAYNQKLRD

SEQ ID NO: 1097 - protein, 9 aa, synthetic construct
  source 1..9: note = Antibody LK-RB2-42 CDR-H3
LNWDEGFAY

SEQ ID NO: 1098 - protein, 16 aa, synthetic construct
  source 1..16: note = Antibody LK-RB2-42 CDR-L1
RSSQSIVHSNGNTYLE

SEQ ID NO: 1099 - protein, 7 aa, synthetic construct
  source 1..7: note = Antibody LK-RB2-42 CDR-L2
KVSNRFS

SEQ ID NO: 1100 - protein, 9 aa, synthetic construct
  source 1..9: note = Antibody LK-RB2-42 CDR-L3
FQGSLVPPT

SEQ ID NO: 1101 - protein, 5 aa, synthetic construct
  source 1..5: note = Antibody LK-RB2-43 CDR-H1
SYGMS

SEQ ID NO: 1102 - protein, 17 aa, synthetic construct
  source 1..17: note = Antibody LK-RB2-43 CDR-H2
TISGGGTYTYYPDSVKG

SEQ ID NO: 1103 - protein, 5 aa, synthetic construct
  source 1..5: note = Antibody LK-RB2-43 CDR-H3
GAMDY

SEQ ID NO: 1104 - protein, 10 aa, synthetic construct
  source 1..10: note = Antibody LK-RB2-43 CDR-L1
SANSSVSSMY

SEQ ID NO: 1105 - protein, 7 aa, synthetic construct
  source 1..7: note = Antibody LK-RB2-43 CDR-L2
GTSNLAS

SEQ ID NO: 1106 - protein, 9 aa, synthetic construct
  source 1..9: note = Antibody LK-RB2-43 CDR-L3
QQYHSHPLT

SEQ ID NO: 1107 - protein, 5 aa, synthetic construct
  source 1..5: note = Antibody LK-RB2-44 CDR-H1
SYGMS

SEQ ID NO: 1108 - protein, 17 aa, synthetic construct
  source 1..17: note = Antibody LK-RB2-44 CDR-H2
TISGGGTYTYYPDSVKG

SEQ ID NO: 1109 - protein, 5 aa, synthetic construct
  source 1..5: note = Antibody LK-RB2-44 CDR-H3
GAMDY

SEQ ID NO: 1110 - protein, 16 aa, synthetic construct
  source 1..16: note = Antibody LK-RB2-44 CDR-L1
KSSQSLLDIDGKTFLN

SEQ ID NO: 1111 - protein, 7 aa, synthetic construct
  source 1..7: note = Antibody LK-RB2-44 CDR-L2
LVSKLDS

SEQ ID NO: 1112 - protein, 9 aa, synthetic construct
  source 1..9: note = Antibody LK-RB2-44 CDR-L3
WQGTHFPWT

SEQ ID NO: 1113 - protein, 5 aa, synthetic construct
  source 1..5: note = Antibody LK-RB2-45 CDR-H1
TYGMS

SEQ ID NO: 1114 - protein, 17 aa, synthetic construct
  source 1..17: note = Antibody LK-RB2-45 CDR-H2
WINVYSGVPTYADDFKG

SEQ ID NO: 1115 - protein, 11 aa, synthetic construct
  source 1..11: note = Antibody LK-RB2-45 CDR-H3
RGDYDGHYFDY

SEQ ID NO: 1116 - protein, 11 aa, synthetic construct
  source 1..11: note = Antibody LK-RB2-45 CDR-L1
RASQSISDYLN

SEQ ID NO: 1117 - protein, 7 aa, synthetic construct
  source 1..7: note = Antibody LK-RB2-45 CDR-L2
YVSQSIS

SEQ ID NO: 1118 - protein, 9 aa, synthetic construct
  source 1..9: note = Antibody LK-RB2-45 CDR-L3
QNGHNFPLT

SEQ ID NO: 1119 - protein, 5 aa, synthetic construct
  source 1..5: note = Antibody LK-RB2-46 CDR-H1
DQIIH

SEQ ID NO: 1120 - protein, 17 aa, synthetic construct
  source 1..17: note = Antibody LK-RB2-46 CDR-H2
YIYPRDDSIMYNEKFKG

SEQ ID NO: 1121 - protein, 8 aa, synthetic construct
  source 1..8: note = Antibody LK-RB2-46 CDR-H3
DAYWYFDV

SEQ ID NO: 1122 - protein, 11 aa, synthetic construct
  source 1..11: note = Antibody LK-RB2-46 CDR-L1
KASQDVNTAVA

SEQ ID NO: 1123 - protein, 7 aa, synthetic construct
  source 1..7: note = Antibody LK-RB2-46 CDR-L2
WASTRHT

SEQ ID NO: 1124 - protein, 9 aa, synthetic construct
  source 1..9: note = Antibody LK-RB2-46 CDR-L3
QQHYSPPLT

SEQ ID NO: 1125 - protein, 5 aa, synthetic construct
  source 1..5: note = Antibody LK-RB2-47 CDR-H1
SYGVH

SEQ ID NO: 1126 - protein, 16 aa, synthetic construct
  source 1..16: note = Antibody LK-RB2-47 CDR-H2
VIWSGGSTDYNAAFIS

SEQ ID NO: 1127 - protein, 11 aa, synthetic construct
  source 1..11: note = Antibody LK-RB2-47 CDR-H3
SFHYHGSTFDY

SEQ ID NO: 1128 - protein, 11 aa, synthetic construct
  source 1..11: note = Antibody LK-RB2-47 CDR-L1
RASQDINNYLN

SEQ ID NO: 1129 - protein, 7 aa, synthetic construct
  source 1..7: note = Antibody LK-RB2-47 CDR-L2
FTSRLHS

SEQ ID NO: 1130 - protein, 9 aa, synthetic construct
  source 1..9: note = Antibody LK-RB2-47 CDR-L3
QQVNTLPYT

SEQ ID NO: 1131 - protein, 449 aa, synthetic construct
  source 1..449: note = Antibody LK-RB1-9.h1 Heavy chain
QVQLVQSGSELKKPGASVKVSCKASGYTFTTYGMSWVRQAPGQGLEWMGWINTYSGVPTYADDFKGRFVFSLDTSVSTAYLQISSLKAEDTAVYYCARRRRTYHGYLSFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 1132 - protein, 214 aa, synthetic construct
  source 1..214: note = Antibody LK-RB1-9.h1 Light chain
DIQMTQSPSSLSASVGDRVTITCETSENIYGALNWYQQKPGKAPKLLIYGATNLAEGVPSRFSGSGSGTDFTFTISSLQPEDIATYYCQNVLNTPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1133 - protein, 449 aa, synthetic construct
  source 1..449: note = Antibody LK-RB1-9.h2 Heavy chain
QVQLVQSGSELKKPGASVKVSCTASGYTFTTYGMSWVRQAPGQGLKWMGWINTYSGVPTYADDFKGRFVFSLDTSVSTAYLQISSLKAEDTAVYFCARRRRTYHGYLSFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 1134 - protein, 214 aa, synthetic construct
  source 1..214: note = Antibody LK-RB1-9.h2 Light chain
DIQMTQSPSSLSASVGDRVTITCETSENIYGALNWYQQKPGKAPKLLIYGATNLAEGVPSRFSGSGSGTDYTFTISSLQPEDIATYYCQNVLNTPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1135 - protein, 449 aa, synthetic construct
  source 1..449: note = Antibody LK-RB1-9.h3 Heavy chain
QVQLVQSGSELKKPGASVKVSCTASGYTFTTYGMSWVKQAPGQGLKWMGWINTYSGVPTYADDFKGRFVFSLDTSVSTAYLQISSLKAEDTAVYFCARRRRTYHGYLSFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 1136 - protein, 214 aa, synthetic construct; identical to SEQ ID NO: 1134
  source 1..214: note = Antibody LK-RB1-9.h3 Light chain

SEQ ID NO: 1137 - protein, 445 aa, synthetic construct
  source 1..445: note = Antibody LK-RB1-16.h1 Heavy chain
QVQLQESGPGLVKPSETLSLTCTVSGFSLISNAISWIRQPPGKGLEWLGVIWGDGNTIYHSALRSRVTISKDTSKNQFSLKLSSVTAADTAVYYCAKANWDGYYFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 1138 - protein, 214 aa, synthetic construct
  source 1..214: note = Antibody LK-RB1-16.h1 Light chain
DIQMTQSPSTLSASVGDRVTITCHASQSVDFWLNWYQQKPGKAPKLLIYKASNLHTGVPSRFSGSGSGTEFTLTISSLQPDDFATYYCQQGQSYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1139 - protein, 445 aa, synthetic construct
  source 1..445: note = Antibody LK-RB1-16.h2 Heavy chain
QVQLQESGPGLVKPSETLSLTCTVSGFSLISNAISWIRQPPGKGLEWLGVIWGDGNTIYHSALRSRLTISKDTSKNQVFLKLSSVTAADTAVYYCAKANWDGYYFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 1140 - protein, 214 aa, synthetic construct; identical to SEQ ID NO: 1138
  source 1..214: note = Antibody LK-RB1-16.h2 Light chain

SEQ ID NO: 1141 - protein, 445 aa, synthetic construct; identical to SEQ ID NO: 1139
  source 1..445: note = Antibody LK-RB1-16.h3 Heavy chain

SEQ ID NO: 1142 - protein, 214 aa, synthetic construct
  source 1..214: note = Antibody LK-RB1-16.h3 Light chain
DIQMTQSPSTLSASVGDRVTITCHASQSVDFWLNWYQQKPGKAPKLLISKASNLHTGVPSRFSGSGSGTEFTLTISSLQPDDFATYYCQQGQSYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1143 - protein, 451 aa, synthetic construct
  source 1..451: note = Antibody LK-RB2-2.h1 Heavy chain
EVQLVESGGGLVKPGGSLRLSCAASGFTFSSYAMSWVRQAPGKGLEWVSYISSGGDYFHYADTVKGRFTISRDNAKNSLYLQMNSLRAEDTAVYYCAREGAHYDSSYLAWFAHWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 1144 - protein, 214 aa, synthetic construct
  source 1..214: note = Antibody LK-RB2-2.h1 Light chain
DIQMTQSPSSVSASVGDRVTITCRASENIYSNLAWYQQKPGKAPKLLIYAATNLAEGVPSRFSGSGSGTDYTLTISSLQPEDFATYYCQQFWDNPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1145 - protein, 451 aa, synthetic construct; identical to SEQ ID NO: 1143
  source 1..451: note = Antibody LK-RB2-2.h2 Heavy chain

SEQ ID NO: 1146 - protein, 214 aa, synthetic construct
  source 1..214: note = Antibody LK-RB2-2.h2 Light chain
DIQMTQSPSSVSASVGDRVTITCRASENIYSNLAWYQQKPGKAPKLLVYAATNLAEGVPSRFSGSGSGTDYTLTISSLQPEDFATYYCQQFWDNPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1147 - protein, 451 aa, synthetic construct
  source 1..451: note = Antibody LK-RB2-2.h3 Heavy chain
EVQLVESGGGLVKPGGSLRLSCAASGFTFSSYAMSWVRQAPGKGLEWVAYISSGGDYFHYADTVKGRFTISRDNAKNSLYLQMNSLRAEDTAVYYCTREGAHYDSSYLAWFAHWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 1148 - protein, 214 aa, synthetic construct; identical to SEQ ID NO: 1144
  source 1..214: note = Antibody LK-RB2-2.h3 Light chain

SEQ ID NO: 1149 - protein, 447 aa, synthetic construct
  source 1..447: note = Antibody LK-RB2-24.h1 Heavy chain
QVQLVQSGSELKKPGASVKVSCKASGYTFTTYGMSWVKQAPGQGLKWMGWLNIYSGVPAYADDFKGRFVFSLDTSASTAYLQISSLKAEDTAVYFCARRENYDGYGMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 1150 - protein, 214 aa, synthetic construct
  source 1..214: note = Antibody LK-RB2-24.h1 Light chain
EIVLTQSPATLSLSPGERATLSCRASQSISDYLHWYQQKPGQAPRLLIKYASQSISGIPARFSGSGSGTDFTLTISSLEPEDFAVYYCQNGHSFPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1151 - protein, 447 aa, synthetic construct; identical to SEQ ID NO: 1149
  source 1..447: note = Antibody LK-RB2-24.h2 Heavy chain

SEQ ID NO: 1152 - protein, 214 aa, synthetic construct
  source 1..214: note = Antibody LK-RB2-24.h2 Light chain
EIVLTQSPATLSLSPGERATLSCRASQSISDYLHWYQQKPGQAPRLLIKYASQSISGIPSRFSGSGSGTDFTLTISSLEPEDFAVYYCQNGHSFPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1153 - protein, 447 aa, synthetic construct; identical to SEQ ID NO: 1149
  source 1..447: note = Antibody LK-RB2-24.h3 Heavy chain

SEQ ID NO: 1154 - protein, 214 aa, synthetic construct
  source 1..214: note = Antibody LK-RB2-24.h3 Light chain
EIVLTQSPATLSLSPGERATLSCRASQSISDYLHWYQQKPGQAPRLLIKYASQSISGIPSRFSGSGSGTDFTLTISSLEPEDVAVYYCQNGHSFPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1155 - protein, 122 aa, synthetic construct
  source 1..122: note = Antibody LK-RB1-9.h1 VH
QVQLVQSGSELKKPGASVKVSCKASGYTFTTYGMSWVRQAPGQGLEWMGWINTYSGVPTYADDFKGRFVFSLDTSVSTAYLQISSLKAEDTAVYYCARRRRTYHGYLSFDYWGQGTTVTVSS

SEQ ID NO: 1156 - protein, 107 aa, synthetic construct
  source 1..107: note = Antibody LK-RB1-9.h1 VL
DIQMTQSPSSLSASVGDRVTITCETSENIYGALNWYQQKPGKAPKLLIYGATNLAEGVPSRFSGSGSGTDFTFTISSLQPEDIATYYCQNVLNTPWTFGGGTKLEIK

SEQ ID NO: 1157 - protein, 122 aa, synthetic construct
  source 1..122: note = Antibody LK-RB1-9.h2 VH
QVQLVQSGSELKKPGASVKVSCTASGYTFTTYGMSWVRQAPGQGLKWMGWINTYSGVPTYADDFKGRFVFSLDTSVSTAYLQISSLKAEDTAVYFCARRRRTYHGYLSFDYWGQGTTVTVSS

SEQ ID NO: 1158 - protein, 107 aa, synthetic construct
  source 1..107: note = Antibody LK-RB1-9.h2 VL
DIQMTQSPSSLSASVGDRVTITCETSENIYGALNWYQQKPGKAPKLLIYGATNLAEGVPSRFSGSGSGTDYTFTISSLQPEDIATYYCQNVLNTPWTFGGGTKLEIK

SEQ ID NO: 1159 - protein, 122 aa, synthetic construct
  source 1..122: note = Antibody LK-RB1-9.h3 VH
QVQLVQSGSELKKPGASVKVSCTASGYTFTTYGMSWVKQAPGQGLKWMGWINTYSGVPTYADDFKGRFVFSLDTSVSTAYLQISSLKAEDTAVYFCARRRRTYHGYLSFDYWGQGTTVTVSS

SEQ ID NO: 1160 - protein, 107 aa, synthetic construct; identical to SEQ ID NO: 1158
  source 1..107: note = Antibody LK-RB1-9.h3 VL

SEQ ID NO: 1161 - protein, 118 aa, synthetic construct
  source 1..118: note = Antibody LK-RB1-16.h1 VH
QVQLQESGPGLVKPSETLSLTCTVSGFSLISNAISWIRQPPGKGLEWLGVIWGDGNTIYHSALRSRVTISKDTSKNQFSLKLSSVTAADTAVYYCAKANWDGYYFDYWGQGTTVTVSS

SEQ ID NO: 1162 - protein, 107 aa, synthetic construct
  source 1..107: note = Antibody LK-RB1-16.h1 VL
DIQMTQSPSTLSASVGDRVTITCHASQSVDFWLNWYQQKPGKAPKLLIYKASNLHTGVPSRFSGSGSGTEFTLTISSLQPDDFATYYCQQGQSYPLTFGQGTKLEIK

SEQ ID NO: 1163 - protein, 118 aa, synthetic construct
  source 1..118: note = Antibody LK-RB1-16.h2 VH
QVQLQESGPGLVKPSETLSLTCTVSGFSLISNAISWIRQPPGKGLEWLGVIWGDGNTIYHSALRSRLTISKDTSKNQVFLKLSSVTAADTAVYYCAKANWDGYYFDYWGQGTTVTVSS

SEQ ID NO: 1164 - protein, 107 aa, synthetic construct; identical to SEQ ID NO: 1162
  source 1..107: note = Antibody LK-RB1-16.h2 VL

SEQ ID NO: 1165 - protein, 118 aa, synthetic construct; identical to SEQ ID NO: 1163
  source 1..118: note = Antibody LK-RB1-16.h3 VH

SEQ ID NO: 1166 - protein, 107 aa, synthetic construct
  source 1..107: note = Antibody LK-RB1-16.h3 VL
DIQMTQSPSTLSASVGDRVTITCHASQSVDFWLNWYQQKPGKAPKLLISKASNLHTGVPSRFSGSGSGTEFTLTISSLQPDDFATYYCQQGQSYPLTFGQGTKLEIK

SEQ ID NO: 1167 - protein, 124 aa, synthetic construct
  source 1..124: note = Antibody LK-RB2-2.h1 VH
EVQLVESGGGLVKPGGSLRLSCAASGFTFSSYAMSWVRQAPGKGLEWVSYISSGGDYFHYADTVKGRFTISRDNAKNSLYLQMNSLRAEDTAVYYCAREGAHYDSSYLAWFAHWGQGTLVTVSS

SEQ ID NO: 1168 - protein, 107 aa, synthetic construct
  source 1..107: note = Antibody LK-RB2-2.h1 VL
DIQMTQSPSSVSASVGDRVTITCRASENIYSNLAWYQQKPGKAPKLLIYAATNLAEGVPSRFSGSGSGTDYTLTISSLQPEDFATYYCQQFWDNPLTFGQGTKLEIK

SEQ ID NO: 1169 - protein, 124 aa, synthetic construct; identical to SEQ ID NO: 1167
  source 1..124: note = Antibody LK-RB2-2.h2 VH

SEQ ID NO: 1170 - protein, 107 aa, synthetic construct
  source 1..107: note = Antibody LK-RB2-2.h2 VL
DIQMTQSPSSVSASVGDRVTITCRASENIYSNLAWYQQKPGKAPKLLVYAATNLAEGVPSRFSGSGSGTDYTLTISSLQPEDFATYYCQQFWDNPLTFGQGTKLEIK

SEQ ID NO: 1171 - protein, 124 aa, synthetic construct
  source 1..124: note = Antibody LK-RB2-2.h3 VH
EVQLVESGGGLVKPGGSLRLSCAASGFTFSSYAMSWVRQAPGKGLEWVAYISSGGDYFHYADTVKGRFTISRDNAKNSLYLQMNSLRAEDTAVYYCTREGAHYDSSYLAWFAHWGQGTLVTVSS

SEQ ID NO: 1172 - protein, 107 aa, synthetic construct; identical to SEQ ID NO: 1168
  source 1..107: note = Antibody LK-RB2-2.h3 VL

SEQ ID NO: 1173 - protein, 120 aa, synthetic construct
  source 1..120: note = Antibody LK-RB2-24.h1 VH
QVQLVQSGSELKKPGASVKVSCKASGYTFTTYGMSWVKQAPGQGLKWMGWLNIYSGVPAYADDFKGRFVFSLDTSASTAYLQISSLKAEDTAVYFCARRENYDGYGMDYWGQGTLVTVSS

SEQ ID NO: 1174 - protein, 107 aa, synthetic construct
  source 1..107: note = Antibody LK-RB2-24.h1 VL
EIVLTQSPATLSLSPGERATLSCRASQSISDYLHWYQQKPGQAPRLLIKYASQSISGIPARFSGSGSGTDFTLTISSLEPEDFAVYYCQNGHSFPYTFGGGTKLEIK

SEQ ID NO: 1175 - protein, 120 aa, synthetic construct; identical to SEQ ID NO: 1173
  source 1..120: note = Antibody LK-RB2-24.h2 VH

SEQ ID NO: 1176 - protein, 107 aa, synthetic construct
  source 1..107: note = Antibody LK-RB2-24.h2 VL
EIVLTQSPATLSLSPGERATLSCRASQSISDYLHWYQQKPGQAPRLLIKYASQSISGIPSRFSGSGSGTDFTLTISSLEPEDFAVYYCQNGHSFPYTFGGGTKLEIK

SEQ ID NO: 1177 - protein, 120 aa, synthetic construct; identical to SEQ ID NO: 1173
  source 1..120: note = Antibody LK-RB2-24.h3 VH

SEQ ID NO: 1178 - protein, 107 aa, synthetic construct
  source 1..107: note = Antibody LK-RB2-24.h3 VL
EIVLTQSPATLSLSPGERATLSCRASQSISDYLHWYQQKPGQAPRLLIKYASQSISGIPSRFSGSGSGTDFTLTISSLEPEDVAVYYCQNGHSFPYTFGGGTKLEIK

SEQ ID NO: 1179 - protein, 13 aa, synthetic construct
  source 1..13: note = Antibody LK-RB1-9.h1, LK-RB1-9.h2. LK-RB1-9.h3 CDR-H3
RRRTYHGYLSFDY

SEQ ID NO: 1180 - protein, 707 aa, synthetic construct
  source 1..707: note = Bispecific antibody LK-RBi-01 Heavy chain
QVQLVQSGSELKKPGASVKVSCKASGYTFTTYGMSWVRQAPGQGLEWMGWINTYSGVPTYADDFKGRFVFSLDTSVSTAYLQISSLKAEDTAVYYCARRRRTYHGYLSFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGSGGGGSGSGGGGSQVQLVQSGSELKKPGASVKVSCKASGYTFTTYGMSWVKQAPGQGLKWMGWLNIYSGVPAYADDFKGRFVFSLDTSASTAYLQISSLKAEDTAVYFCARRENYDGYGMDYWGQGTLVTVSSGSGGGGSGSGGGGSGSEIVLTQSPATLSLSPGERATLSCRASQSISDYLHWYQQKPGQAPRLLIKYASQSISGIPSRFSGSGSGTDFTLTISSLEPEDVAVYYCQNGHSFPYTFGGGTKLEIK

SEQ ID NO: 1181 - protein, 214 aa, synthetic construct; identical to SEQ ID NO: 1132
  source 1..214: note = Bispecific antibody LK-RBi-01 Light chain

SEQ ID NO: 1182 - protein, 711 aa, synthetic construct
  source 1..711: note = Bispecific antibody LK-RBi-02 Heavy chain
QVQLVQSGSELKKPGASVKVSCKASGYTFTTYGMSWVRQAPGQGLEWMGWINTYSGVPTYADDFKGRFVFSLDTSVSTAYLQISSLKAEDTAVYYCARRRRTYHGYLSFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGSGGGGSGSGGGGSEVQLVESGGGLVKPGGSLRLSCAASGFTFSSYAMSWVRQAPGKGLEWVSYISSGGDYFHYADTVKGRFTISRDNAKNSLYLQMNSLRAEDTAVYYCAREGAHYDSSYLAWFAHWGQGTLVTVSSGSGGGGSGSGGGGSGSDIQMTQSPSSVSASVGDRVTITCRASENIYSNLAWYQQKPGKAPKLLIYAATNLAEGVPSRFSGSGSGTDYTLTISSLQPEDFATYYCQQFWDNPLTFGQGTKLEIK

SEQ ID NO: 1183 - protein, 214 aa, synthetic construct; identical to SEQ ID NO: 1132
  source 1..214: note = Bispecific antibody LK-RBi-02 Light chain

SEQ ID NO: 1184 - protein, 711 aa, synthetic construct
  source 1..711: note = Bispecific antibody LK-RBi-03 Heavy chain
EVQLVESGGGLVKPGGSLRLSCAASGFTFSSYAMSWVRQAPGKGLEWVSYISSGGDYFHYADTVKGRFTISRDNAKNSLYLQMNSLRAEDTAVYYCAREGAHYDSSYLAWFAHWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGSGGGGSGSGGGGSQVQLVQSGSELKKPGASVKVSCKASGYTFTTYGMSWVRQAPGQGLEWMGWINTYSGVPTYADDFKGRFVFSLDTSVSTAYLQISSLKAEDTAVYYCARRRRTYHGYLSFDYWGQGTTVTVSSGSGGGGSGSGGGGSGSDIQMTQSPSSLSASVGDRVTITCETSENIYGALNWYQQKPGKAPKLLIYGATNLAEGVPSRFSGSGSGTDFTFTISSLQPEDIATYYCQNVLNTPWTFGGGTKLEIK

SEQ ID NO: 1185 - protein, 214 aa, synthetic construct; identical to SEQ ID NO: 1144
  source 1..214: note = Bispecific antibody LK-RBi-03 Light chain

SEQ ID NO: 1186 - protein, 707 aa, synthetic construct
  source 1..707: note = Bispecific antibody LK-RBi-04 Heavy chain
QVQLVQSGSELKKPGASVKVSCKASGYTFTTYGMSWVKQAPGQGLKWMGWLNIYSGVPAYADDFKGRFVFSLDTSASTAYLQISSLKAEDTAVYFCARRENYDGYGMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGSGGGGSGSGGGGSQVQLVQSGSELKKPGASVKVSCKASGYTFTTYGMSWVRQAPGQGLEWMGWINTYSGVPTYADDFKGRFVFSLDTSVSTAYLQISSLKAEDTAVYYCARRRRTYHGYLSFDYWGQGTTVTVSSGSGGGGSGSGGGGSGSDIQMTQSPSSLSASVGDRVTITCETSENIYGALNWYQQKPGKAPKLLIYGATNLAEGVPSRFSGSGSGTDFTFTISSLQPEDIATYYCQNVLNTPWTFGGGTKLEIK

SEQ ID NO: 1187 - protein, 214 aa, synthetic construct; identical to SEQ ID NO: 1154
  source 1..214: note = Bispecific antibody LK-RBi-04 Light chain

SEQ ID NO: 1188 - protein, 582 aa, synthetic construct
  source 1..582: note = Bispecific antibody LK-RBi-05 Heavy chain
QVQLVQSGSELKKPGASVKVSCKASGYTFTTYGMSWVRQAPGQGLEWMGWINTYSGVPTYADDFKGRFVFSLDTSVSTAYLQISSLKAEDTAVYYCARRRRTYHGYLSFDYWGQGTTVTVSSGGSGGGGSGEVQLVESGGGLVKPGGSLRLSCAASGFTFSSYAMSWVRQAPGKGLEWVSYISSGGDYFHYADTVKGRFTISRDNAKNSLYLQMNSLRAEDTAVYYCAREGAHYDSSYLAWFAHWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 1189 - protein, 330 aa, synthetic construct
  source 1..330: note = Bispecific antibody LK-RBi-05 Light chain
DIQMTQSPSSLSASVGDRVTITCETSENIYGALNWYQQKPGKAPKLLIYGATNLAEGVPSRFSGSGSGTDFTFTISSLQPEDIATYYCQNVLNTPWTFGGGTKLEIKGGSGGGGSGDIQMTQSPSSVSASVGDRVTITCRASENIYSNLAWYQQKPGKAPKLLIYAATNLAEGVPSRFSGSGSGTDYTLTISSLQPEDFATYYCQQFWDNPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1190 - protein, 711 aa, synthetic construct
  source 1..711: note = Bispecific antibody LK-RBi-06 Heavy chain
QVQLQESGPGLVKPSETLSLTCTVSGFSLISNAISWIRQPPGKGLEWLGVIWGDGNTIYHSALRSRLTISKDTSKNQVFLKLSSVTAADTAVYYCAKANWDGYYFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSGGGGSEVQLVESGGGLVKPGGSLRLSCAASGFTFSSYAMSWVRQAPGKGLEWVSYISSGGDYFHYADTVKGRFTISRDNAKNSLYLQMNSLRAEDTAVYYCAREGAHYDSSYLAWFAHWGQGTLVTVSSGGGGSGGGGSGGGGSDIQMTQSPSSVSASVGDRVTITCRASENIYSNLAWYQQKPGKAPKLLIYAATNLAEGVPSRFSGSGSGTDYTLTISSLQPEDFATYYCQQFWDNPLTFGQGTKLEIK

SEQ ID NO: 1191 - protein, 214 aa, synthetic construct; identical to SEQ ID NO: 1142
  source 1..214: note = Bispecific antibody LK-RBi-06 Light chain

SEQ ID NO: 1192 - protein, 707 aa, synthetic construct
  source 1..707: note = Bispecific antibody LK-RBi-07 Heavy chain
QVQLQESGPGLVKPSETLSLTCTVSGFSLISNAISWIRQPPGKGLEWLGVIWGDGNTIYHSALRSRLTISKDTSKNQVFLKLSSVTAADTAVYYCAKANWDGYYFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSGGGGSQVQLVQSGSELKKPGASVKVSCKASGYTFTTYGMSWVKQAPGQGLKWMGWLNIYSGVPAYADDFKGRFVFSLDTSASTAYLQISSLKAEDTAVYFCARRENYDGYGMDYWGQGTLVTVSSGGGGSGGGGSGGGGSEIVLTQSPATLSLSPGERATLSCRASQSISDYLHWYQQKPGQAPRLLIKYASQSISGIPSRFSGSGSGTDFTLTISSLEPEDVAVYYCQNGHSFPYTFGGGTKLEIK

SEQ ID NO: 1193 - protein, 214 aa, synthetic construct; identical to SEQ ID NO: 1142
  source 1..214: note = Bispecific antibody LK-RBi-07 Light chain

SEQ ID NO: 1194 - protein, 707 aa, synthetic construct
  source 1..707: note = Bispecific antibody LK-RBi-08 Heavy chain
EVQLVESGGGLVKPGGSLRLSCAASGFTFSSYAMSWVRQAPGKGLEWVSYISSGGDYFHYADTVKGRFTISRDNAKNSLYLQMNSLRAEDTAVYYCAREGAHYDSSYLAWFAHWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGSGGGGSGSGGGGSDIQMTQSPSTLSASVGDRVTITCHASQSVDFWLNWYQQKPGKAPKLLISKASNLHTGVPSRFSGSGSGTEFTLTISSLQPDDFATYYCQQGQSYPLTFGQGTKLEIKGSGGGGSGSGGGGSGSQVQLQESGPGLVKPSETLSLTCTVSGFSLISNAISWIRQPPGKGLEWLGVIWGDGNTIYHSALRSRLTISKDTSKNQVFLKLSSVTAADTAVYYCAKANWDGYYFDYWGQGTTVTVSS

SEQ ID NO: 1195 - protein, 214 aa, synthetic construct; identical to SEQ ID NO: 1144
  source 1..214: note = Bispecific antibody LK-RBi-08 Light chain

SEQ ID NO: 1196 - protein, 578 aa, synthetic construct
  source 1..578: note = Bispecific antibody LK-RBi-09 Heavy chain
QVQLVQSGSELKKPGASVKVSCKASGYTFTTYGMSWVRQAPGQGLEWMGWINTYSGVPTYADDFKGRFVFSLDTSVSTAYLQISSLKAEDTAVYYCARRRRTYHGYLSFDYWGQGTTVTVSSGGSGGGGSGQVQLVQSGSELKKPGASVKVSCKASGYTFTTYGMSWVKQAPGQGLKWMGWLNIYSGVPAYADDFKGRFVFSLDTSASTAYLQISSLKAEDTAVYFCARRENYDGYGMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 1197 - protein, 330 aa, synthetic construct
  source 1..330: note = Bispecific antibody LK-RBi-09 Light chain
DIQMTQSPSSLSASVGDRVTITCETSENIYGALNWYQQKPGKAPKLLIYGATNLAEGVPSRFSGSGSGTDFTFTISSLQPEDIATYYCQNVLNTPWTFGGGTKLEIKGGSGGGGSGEIVLTQSPATLSLSPGERATLSCRASQSISDYLHWYQQKPGQAPRLLIKYASQSISGIPSRFSGSGSGTDFTLTISSLEPEDVAVYYCQNGHSFPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1198 - protein, 578 aa, synthetic construct
  source 1..578: note = Bispecific antibody LK-RBi-10 Heavy chain
QVQLVQSGSELKKPGASVKVSCKASGYTFTTYGMSWVKQAPGQGLKWMGWLNIYSGVPAYADDFKGRFVFSLDTSASTAYLQISSLKAEDTAVYFCARRENYDGYGMDYWGQGTLVTVSSGGSGGGGSGQVQLVQSGSELKKPGASVKVSCKASGYTFTTYGMSWVRQAPGQGLEWMGWINTYSGVPTYADDFKGRFVFSLDTSVSTAYLQISSLKAEDTAVYYCARRRRTYHGYLSFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 1199 - protein, 330 aa, synthetic construct
  source 1..330: note = Bispecific antibody LK-RBi-10 Light chain
EIVLTQSPATLSLSPGERATLSCRASQSISDYLHWYQQKPGQAPRLLIKYASQSISGIPSRFSGSGSGTDFTLTISSLEPEDVAVYYCQNGHSFPYTFGGGTKLEIKGGSGGGGSGDIQMTQSPSSLSASVGDRVTITCETSENIYGALNWYQQKPGKAPKLLIYGATNLAEGVPSRFSGSGSGTDFTFTISSLQPEDIATYYCQNVLNTPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1200 - protein, 578 aa, synthetic construct
  source 1..578: note = Bispecific antibody LK-RBi-11 Heavy chain
QVQLQESGPGLVKPSETLSLTCTVSGFSLISNAISWIRQPPGKGLEWLGVIWGDGNTIYHSALRSRLTISKDTSKNQVFLKLSSVTAADTAVYYCAKANWDGYYFDYWGQGTTVTVSSGGSGGGGSGEVQLVESGGGLVKPGGSLRLSCAASGFTFSSYAMSWVRQAPGKGLEWVSYISSGGDYFHYADTVKGRFTISRDNAKNSLYLQMNSLRAEDTAVYYCAREGAHYDSSYLAWFAHWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 1201 - protein, 330 aa, synthetic construct
  source 1..330: note = Bispecific antibody LK-RBi-11 Light chain
DIQMTQSPSTLSASVGDRVTITCHASQSVDFWLNWYQQKPGKAPKLLISKASNLHTGVPSRFSGSGSGTEFTLTISSLQPDDFATYYCQQGQSYPLTFGQGTKLEIKGGSGGGGSGDIQMTQSPSSVSASVGDRVTITCRASENIYSNLAWYQQKPGKAPKLLIYAATNLAEGVPSRFSGSGSGTDYTLTISSLQPEDFATYYCQQFWDNPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1202 - protein, 574 aa, synthetic construct
  source 1..574: note = Bispecific antibody LK-RBi-12 Heavy chain
QVQLQESGPGLVKPSETLSLTCTVSGFSLISNAISWIRQPPGKGLEWLGVIWGDGNTIYHSALRSRLTISKDTSKNQVFLKLSSVTAADTAVYYCAKANWDGYYFDYWGQGTTVTVSSGGSGGGGSGQVQLVQSGSELKKPGASVKVSCKASGYTFTTYGMSWVKQAPGQGLKWMGWLNIYSGVPAYADDFKGRFVFSLDTSASTAYLQISSLKAEDTAVYFCARRENYDGYGMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 1203 - protein, 330 aa, synthetic construct
  source 1..330: note = Bispecific antibody LK-RBi-12 Light chain
DIQMTQSPSTLSASVGDRVTITCHASQSVDFWLNWYQQKPGKAPKLLISKASNLHTGVPSRFSGSGSGTEFTLTISSLQPDDFATYYCQQGQSYPLTFGQGTKLEIKGGSGGGGSGEIVLTQSPATLSLSPGERATLSCRASQSISDYLHWYQQKPGQAPRLLIKYASQSISGIPSRFSGSGSGTDFTLTISSLEPEDVAVYYCQNGHSFPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1204 - protein, 650 aa, synthetic construct
  source 1..650: note = LILRB1 Variant 2 sequence
MTPILTVLICLGLSLGPRTHVQAGHLPKPTLWAEPGSVITQGSPVTLRCQGGQETQEYRLYREKKTAPWITRIPQELVKKGQFPIPSITWEHTGRYRCYYGSDTAGRSESSDPLELVVTGAYIKPTLSAQPSPVVNSGGNVTLQCDSQVAFDGFILCKEGEDEHPQCLNSQPHARGSSRAIFSVGPVSPSRRWWYRCYAYDSNSPYEWSLPSDLLELLVLGVSKKPSLSVQPGPIVAPEETLTLQCGSDAGYNRFVLYKDGERDFLQLAGAQPQAGLSQANFTLGPVSRSYGGQYRCYGAHNLSSEWSAPSDPLDILIAGQFYDRVSLSVQPGPTVASGENVTLLCQSQGWMQTFLLTKEGAADDPWRLRSTYQSQKYQAEFPMGPVTSAHAGTYRCYGSQSSKPYLLTHPSDPLELVVSGPSGGPSSPTTGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 1205 - protein, 650 aa, synthetic construct [sequence omitted]

SEQ ID NO: 1206 - protein, 338 aa, Homo sapiens
  source 1..338: note = Human HLA-G (M4T) sequence
MVVTAPRTLFLLLSGALTLTETWAGSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWKQSSLPTIPIMGIVAGLVVLAAVVTGAAVAAVLWRKKSSD

SEQ ID NO: 1207 - protein, 22 aa, synthetic construct
  source 1..22: note = Exemplary signal peptide
MDMRVPAQLLGLLLLWLRGARC

SEQ ID NO: 1208 - protein, 19 aa, synthetic construct
  source 1..19: note = Exemplary signal peptide
MGWLWNLLFLMAAAQSAQA

SEQ ID NO: 1209 - protein, 20 aa, synthetic construct
  source 1..20: note = Exemplary signal peptide
MVFTPQILGLMLFWISASRG

SEQ ID NO: 1210 - protein, 19 aa, synthetic construct
  source 1..19: note = Exemplary signal peptide
MAWTPLFFFFVLHCSGSFS

SEQ ID NO: 1211 - protein, 19 aa, synthetic construct
  source 1..19: note = Exemplary signal peptide
MGVPTQLLLLWLTVVVVRC

SEQ ID NO: 1212 - protein, 19 aa, synthetic construct
  source 1..19: note = Exemplary signal peptide
MGWSCIMLFLAATATGVHS

SEQ ID NO: 1213 - protein, 20 aa, synthetic construct
  source 1..20: note = Exemplary signal peptide
MESQTQVFVYMLLWLSGVDG

SEQ ID NO: 1214 - protein, 19 aa, synthetic construct
  source 1..19: note = Exemplary signal peptide
MGWSYIILFLVATATGVHS

SEQ ID NO: 1215 - protein, 22 aa, synthetic construct
  source 1..22: note = Exemplary signal peptide
MDFQVQIFSFLLISASVILSRG

SEQ ID NO: 1216 - protein, 20 aa, synthetic construct
  source 1..20: note = Exemplary signal peptide
MESQIQAFVFVFLWLSGVDG

SEQ ID NO: 1217 - protein, 22 aa, synthetic construct
  source 1..22: note = Exemplary signal peptide
MNTRAPAEFLGFLLLWFLGARC

SEQ ID NO: 1218 - protein, 19 aa, synthetic construct
  source 1..19: note = Exemplary signal peptide
MEWNRVFIFLLSVTAGVHS

SEQ ID NO: 1219 - protein, 20 aa, synthetic construct
  source 1..20: note = Exemplary signal peptide
MEFHTQVFVFVFLWLSGVDG

SEQ ID NO: 1220 - protein, 19 aa, synthetic construct
  source 1..19: note = Exemplary signal peptide
MDRLTSSFLLLIVPAYVLS

SEQ ID NO: 1221 - protein, 20 aa, synthetic construct
  source 1..20: note = Exemplary signal peptide
MVSTPQFLVFLLFWIPASRG

SEQ ID NO: 1222 - protein, 19 aa, synthetic construct
  source 1..19: note = Exemplary signal peptide
MGRLTSSFLLLIVPAYVLS

SEQ ID NO: 1223 - protein, 20 aa, synthetic construct
  source 1..20: note = Exemplary signal peptide
METDTLLLWVLLLWVPGSTG

SEQ ID NO: 1224 - protein, 19 aa, synthetic construct
  source 1..19: note = Exemplary signal peptide
MEWPCIFLFLLSVTEGVHS

SEQ ID NO: 1225 - protein, 19 aa, synthetic construct
  source 1..19: note = Exemplary signal peptide
MAVLALLLCLVTFPSCVLS

SEQ ID NO: 1226 - protein, 20 aa, synthetic construct
  source 1..20: note = Exemplary signal peptide
MRVLAELLGLLLFCFLGVRC

SEQ ID NO: 1227 - protein, 19 aa, synthetic construct
  source 1..19: note = Exemplary signal peptide
MSSAQFLGLLLLCFQGTRC

SEQ ID NO: 1228 - protein, 19 aa, synthetic construct
  source 1..19: note = Exemplary signal peptide
MGWSCIILILVVAATGVHS

SEQ ID NO: 1229 - protein, 19 aa, synthetic construct
  source 1..19: note = Exemplary signal peptide
MGWSCIILFLVATATGVHS

SEQ ID NO: 1230 - protein, 20 aa, synthetic construct
  source 1..20: note = Exemplary signal peptide
MSVPTQVLGLLLLWLTDARC

SEQ ID NO: 1231 - protein, 19 aa, synthetic construct
  source 1..19: note = Exemplary signal peptide
MSPAQFLFLLVLWIRENNG

SEQ ID NO: 1232 - protein, 19 aa, synthetic construct
  source 1..19: note = Exemplary signal peptide
MGWLWNLLFLMAAAQSAQT

SEQ ID NO: 1233 - protein, 19 aa, synthetic construct
  source 1..19: note = Exemplary signal peptide
MERHWIFLSLLSVIAGVHS

SEQ ID NO: 1234 - protein, 20 aa, synthetic construct
  source 1..20: note = Exemplary signal peptide
MSVPTQVLGLLLLWLTDVRC

SEQ ID NO: 1235 - protein, 19 aa, synthetic construct
  source 1..19: note = Exemplary signal peptide
MELCWVFLFLLPVTAGVHS

SEQ ID NO: 1236 - protein, 18 aa, synthetic construct
  source 1..18: note = Exemplary signal peptide
MKVLSLLYLLTAIPGILS

SEQ ID NO: 1237 - protein, 19 aa, synthetic construct
  source 1..19: note = Exemplary signal peptide
MGWSCIMLFLAATATGVRS

SEQ ID NO: 1238 - protein, 19 aa, synthetic construct
  source 1..19: note = Exemplary signal peptide
MKCSWVIFFLMAVVIGVNS

SEQ ID NO: 1239 - protein, 22 aa, synthetic construct
  source 1..22: note = Exemplary signal peptide
MHFQVQIFSFLLISASVIMSRG

SEQ ID NO: 1240 - protein, 19 aa, synthetic construct
  source 1..19: note = Exemplary signal peptide
MKLPVRLLVLMFWIPASSS

SEQ ID NO: 1241 - protein, 19 aa, synthetic construct
  source 1..19: note = Exemplary signal peptide
MGWSYVILFLVATATGVHS

SEQ ID NO: 1242 - protein, 19 aa, synthetic construct
  source 1..19: note = Exemplary signal peptide
MGWSWIFLFLLSGTAGVHS

SEQ ID NO: 1243 - protein, 19 aa, synthetic construct
  source 1..19: note = Exemplary signal peptide
MGWSWIFLFLLSGTAGVLS

SEQ ID NO: 1244 - protein, 20 aa, synthetic construct
  source 1..20: note = Exemplary signal peptide
METETLLLWVLLLWVPGSTG

SEQ ID NO: 1245 - protein, 19 aa, synthetic construct
  source 1..19: note = Exemplary signal peptide
MNLGLSLIFLVLVLKGVQC

SEQ ID NO: 1246 - protein, 19 aa, synthetic construct
  source 1..19: note = Exemplary signal peptide
MAVLVLLLCLVTFPSCVLS

SEQ ID NO: 1247 - protein, 22 aa, synthetic construct
  source 1..22: note = Exemplary signal peptide
MDFQVQIFSFLLISASVIMSRG

SEQ ID NO: 1248 - protein, 19 aa, synthetic construct
  source 1..19: note = Exemplary signal peptide
MEWCWVFLFLLSVTAGVHS

SEQ ID NO: 1249 - protein, 22 aa, synthetic construct
  source 1..22: note = Exemplary signal peptide
MDFQVQIFSFLLISISVVMSRG

SEQ ID NO: 1250 - protein, 19 aa, synthetic construct
  source 1..19: note = Exemplary signal peptide
MAWLWNLLFLMAAAQSAQA

SEQ ID NO: 1251 - protein, 18 aa, synthetic construct
  source 1..18: note = Exemplary signal peptide
MKVLSLLYLLTAIPGFLS

SEQ ID NO: 1252 - protein, 20 aa, synthetic construct
  source 1..20: note = Exemplary signal peptide
MDSQAQVLMLLLLWVSGTCG

SEQ ID NO: 1253 - protein, 19 aa, synthetic construct
  source 1..19: note = Exemplary signal peptide
MNFGLRLIFLVLTLKGVQC

SEQ ID NO: 1254 - protein, 19 aa, synthetic construct
  source 1..19: note = Exemplary signal peptide
MEWSWVSLFFLSVTTGVHS

SEQ ID NO: 1255 - protein, 20 aa, synthetic construct
  source 1..20: note = Exemplary signal peptide
MRTPAQFLGILLLWFPGIKC

SEQ ID NO: 1256 - protein, 19 aa, synthetic construct
  source 1..19: note = Exemplary signal peptide
MNFGLSLIFLALILKGVQC

SEQ ID NO: 1257 - protein, 20 aa, synthetic construct
  source 1..20: note = Exemplary signal peptide
MVSTSQLLGLLLFWTSASRC

SEQ ID NO: 1258 - protein, 20 aa, synthetic construct
  source 1..20: note = Exemplary signal peptide
MSVPTQVLGLLLLWLTGARC

SEQ ID NO: 1259 - protein, 19 aa, synthetic construct
  source 1..19: note = Exemplary signal peptide
MNFGLSLIFLVLILKGVQC

SEQ ID NO: 1260 - protein, 20 aa, synthetic construct
  source 1..20: note = Exemplary signal peptide
MEFRTQVFVFVFLWLSGVDG

SEQ ID NO: 1261 - protein, 22 aa, synthetic construct
  source 1..22: note = Exemplary signal peptide
MDMRTPAQFLGILLLWFPGIKC

SEQ ID NO: 1262 - protein, 20 aa, synthetic construct
  source 1..20: note = Exemplary signal peptide
METDTLLLWVLLLWVPGSSG

SEQ ID NO: 1263 - protein, 19 aa, synthetic construct
  source 1..19: note = Exemplary signal peptide
MGWRYIILFLVATATGVHS

SEQ ID NO: 1264 - protein, 19 aa, synthetic construct
  source 1..19: note = Exemplary signal peptide
MGWSCIILILVAAATGVHS

SEQ ID NO: 1265 - protein, 20 aa, synthetic construct
  source 1..20: note = Exemplary signal peptide
MESQTLVFISILLWLYGADG

SEQ ID NO: 1266 - protein, 20 aa, synthetic construct
  source 1..20: note = Exemplary signal peptide
MKSQTQVFVFLLLCVSGAHG

SEQ ID NO: 1267 - protein, 19 aa, synthetic construct
  source 1..19: note = Exemplary signal peptide
MAVLVLFLCLVAFPSCVLS

SEQ ID NO: 1268 - protein, 22 aa, synthetic construct
  source 1..22: note = Exemplary signal peptide
MDIRAPAQFLGILLLWFPGARC

SEQ ID NO: 1269 - protein, 19 aa, synthetic construct
  source 1..19: note = Exemplary signal peptide
MVLAQFLAFLLLWFPGARC

SEQ ID NO: 1270 - protein, 19 aa, synthetic construct
  source 1..19: note = Exemplary signal peptide
MECNWILPFILSVTSGVYS

SEQ ID NO: 1271 - protein, 20 aa, synthetic construct
  source 1..20: note = Exemplary signal peptide
MSVLTQVLALLLLWLTGARC

SEQ ID NO: 1272 - protein, 19 aa, synthetic construct
  source 1..19: note = Exemplary signal peptide
MNLGLSFIFLALILKGVQC

SEQ ID NO: 1273 - protein, 19 aa, synthetic construct
  source 1..19: note = Exemplary signal peptide
MKCSWVIFFLMAVVTGVNS

SEQ ID NO: 1274 - protein, 20 aa, synthetic construct
  source 1..20: note = Exemplary signal peptide
MESQIQVFVFVFLWLSGVDG

SEQ ID NO: 1275 - protein, 19 aa, synthetic construct
  source 1..19: note = Exemplary signal peptide
MEWSGVFIFLLSVTAGVHS

SEQ ID NO: 1276 - protein, 19 aa, synthetic construct
  source 1..19: note = Exemplary signal peptide
MNLGLSLIFLVLVLKGGQC

SEQ ID NO: 1277 - protein, 20 aa, synthetic construct
  source 1..20: note = Exemplary signal peptide
MESQTQVLMFLLLWVSGACA

SEQ ID NO: 1278 - protein, 19 aa, synthetic construct
  source 1..19: note = Exemplary signal peptide
MGWSWIFLFLLSETAGVLS

SEQ ID NO: 1279 - protein, 19 aa, synthetic construct
  source 1..19: note = Exemplary signal peptide
MSPAQFLFLLVLWIRETNG

SEQ ID NO: 1280 - protein, 19 aa, synthetic construct
  source 1..19: note = Exemplary signal peptide
MAVLGLLFCLVTLPSCVLS

SEQ ID NO: 1281 - protein, 7 aa, synthetic construct
  source 1..7: note = Antibody LK-RB1-9.h1, LK-RB1-9.h2, LK-RB1-9.h3 CDR-L2
GATNLAE

SEQ ID NO: 1282 - protein, 7 aa, synthetic construct
  source 1..7: note = Antibody LK-RB2-2.h1, LK-RB2-2.h2, LK-RB2-2.h3 CDR-L2
AATNLAE

SEQ ID NO: 1283 - protein, 18 aa, synthetic construct
  source 1..18: note = Exemplary signal peptide
MHSSALLCCLVLLTGVRA

SEQ ID NO: 1284 - protein, 9 aa, synthetic construct
  source 1..9: note = Peptide sequence
RIIPRHLQL

SEQ ID NO: 1285 - protein, 9 aa, synthetic construct
  source 1..9: note = Peptide sequence
AAGIGILTV